(19) United States
(12) Patent Application Publication
(10) Pub. No.: US 2024/0279214 A1
(43) Pub. Date: Aug. 22, 2024

(54) ALPHA4 BETA7 INEGRIN ANTAGONISTS AND USES THEREOF

(71) Applicant: XinThera, Inc., Foster City, CA (US)

(72) Inventors: Robert L. HOFFMAN, San Diego, CA (US); Joseph Robert PINCHMAN, San Diego, CA (US); Porino Jinjo VA, San Diego, CA (US); Qing DONG, San Diego, CA (US)

(21) Appl. No.: 18/393,205

(22) Filed: Dec. 21, 2023

Related U.S. Application Data
(60) Provisional application No. 63/476,861, filed on Dec. 22, 2022.

(51) Int. Cl. C07D 413/14; A61K 31/4427; A61K 31/4545; A61K 31/519; A61K 31/5386; C07D 401/14; C07D 413/04; C07D 471/04; C07D 498/04; C07D 498/08

(52) U.S. Cl. CPC C07D 413/14; A61K 31/4427; A61K 31/4545; A61K 31/519; A61K 31/5386; C07D 401/14; C07D 413/04; C07D 471/04; C07D 498/04; C07D 498/08

(57) ABSTRACT

Described herein are α₄β₇ integrin inhibitors and pharmaceutical compositions comprising said inhibitors. The subject compounds and compositions are useful for the treatment of α₄β₇ integrin-mediated conditions, such as inflammatory bowel disease (IBD), ulcerative colitis (UC), and Crohn's disease (CD).

### CROSS REFERENCE TO RELATED APPLICATIONS

This application claims the benefit under 35 U.S.C. 119(e) of U.S. Provisional Application Ser. No. 63/476,861, filed on Dec. 22, 2022, which is hereby incorporated by reference in its entirety.

### BACKGROUND

Integrins are heterodimeric cell surface receptors composed of noncovalently associated a and f subunits and are involved in numerous cellular processes, including cell adhesion. A cell's adhesive properties can be regulated by the differential expression of integrins, thus allowing different leukocyte populations to be recruited to specific organs in response to inflammatory signals. The α₄β₇ integrin, has been shown to be involved in lymphocyte migration throughout the gut-associated lymphoid tissue (GALT). α₄β₇ is expressed on leukocytes, including T and B lymphocytes, and an important role for α₄β₇ in cell adhesion in GALT has been shown to be mediated via selective binding to its primary ligand, mucosal addressin cell adhesion molecule-1 (MAdCAM-1). For example, memory T lymphocytes (Tₘₑₘ) expressing α₄β₇ integrin preferentially migrate into GALT via adhesion to MAdCAM-1

Inhibitors of integrin-ligand interactions have been used for the treatment of various diseases. For example, a monoclonal antibody (mAb), vedolizumab, displaying high binding affinity for α₄β₇ has demonstrated therapeutic benefits for the treatment of inflammatory bowel diseases (IBD), such as Crohn's disease (CD), and ulcerative colitis (UC). However, mAbs also have certain undesirable properties for the patient. Vedolizumab is administered by parenteral administration, and mAbs in general have long half-lives making it difficult to rapidly modify exposures. Moreover, loss of activity overtime may occur due to anti-drug antibody formation. In addition, some therapies that inhibit α₄ also interfere with α₄β₁ integrin-ligand interactions, specifically the selective binding of α₄β₁ to vascular cell adhesion molecule 1 (VCAM-1), and this inhibition can result in dangerous side effects to the patient. Activity towards α₄β₁ integrin is implicated in the development of progressive multifocal leukoencephalopathy (PML) in patients, a life-threatening and progressive brain disease caused by the JC virus, that is normally held in check by immunosurveillance.

There remains a medical need for effective, selective, and safe oral α₄β₇ integrin inhibitors with improved pharmaceutical properties which target α₄β₇ integrin-mediated conditions, such as inflammatory bowel disease (IBD), such as ulcerative colitis (UC) and Crohn's disease (CD).

### SUMMARY

Disclosed herein is a compound of Formula (I), or a pharmaceutically acceptable salt, solvate, or stereoisomer thereof:

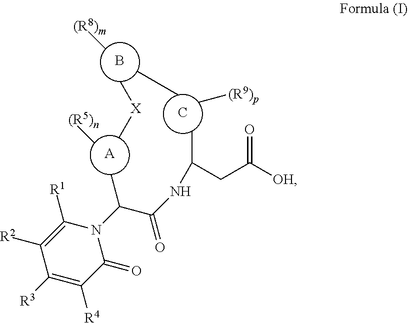

wherein:
R¹ is hydrogen, deuterium, halogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl; wherein the alkyl, cycloalkyl, and heterocycloalkyl is optionally substituted with one or more R;
R² is hydrogen, deuterium, halogen, -L-CN, -L-OH, -L-OR^{a}, -L-S(═O)R^{a}, -L-S(═O)₂R^{a}, -L-S(═O)₂NR^{c}R^{d}, -L-NR^{c}R^{d}, -L-C(═O)R^{a}, -L-C(═O)OR^{b}, -L-C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, -L-cycloalkyl, or -L-heterocycloalkyl; wherein the alkyl, alkenyl, alkynyl, cycloalkyl, and heterocycloalkyl is optionally substituted with one or more R;
R³ is hydrogen, deuterium, halogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl; wherein the alkyl, cycloalkyl, and heterocycloalkyl is optionally substituted with one or more R;
R⁴ is hydrogen, deuterium, halogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl; wherein the alkyl, cycloalkyl, and heterocycloalkyl is optionally substituted with one or more R;
L is absent or C₁-C₆alkylene;
Ring A is aryl or heteroaryl;
each R⁵ is independently halogen, —CN, —NO₂, —OH, —OR^{a}, —SH, —SW, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or two R are taken together to form an oxo;
n is 0-4;
X is —O—, —S—, —C(R⁶)₂—, —NR—, —[C(R⁶)₂]₂—, —O—C(R⁶)₂—, —C(R⁶)₂—O—, —S—C(R⁶)₂—, or —C(R⁶)₂—S—;
each R⁶ is independently hydrogen, deuterium, halogen, —OH, —OR^{a}, C₁-C₆alkyl, C₁-C₆haloalkyl, C₁-C₆hydroxyalkyl, C₁-C₆aminoalkyl, or C₁-C₆heteroalkyl;
or two R⁶ are taken together to form a cycloalkyl or heterocycloalkyl; each optionally substituted with one or more R;
R⁷ is hydrogen, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl;
Ring B is aryl or heteroaryl;
each R⁸ is independently halogen, —CN, —NO₂, —OH, —OR^{a}, —SH, —SR^{a}, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or two R⁸ are taken together to form an oxo;
m is 0-4;
Ring C is aryl or heteroaryl;
each R⁹ is independently halogen, —CN, —NO₂, —OH, —OR^{a}, —SH, —SR^{a}, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or two R⁹ are taken together to form an oxo;
p is 0-4;
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each alkyl, alkylene, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each alkyl, alkylene, alkenyl, alkynyl, cycloalkyl, heterocycloalkyl, aryl, and heteroaryl is independently optionally substituted with one or more R;
R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each alkyl, alkylene, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R; and
each R is independently halogen, —CN, —OH, —OC₁-C₃alkyl, —OC₁-C₃haloalkyl, —SC₁-C₃alkyl, —S(═O)C₁-C₃alkyl, —S(═O)₂C₁-C₃alkyl, —S(═O)₂NH₂, —S(═O)₂NHC₁-C₃alkyl, —S(═O)₂N(C₁-C₃alkyl)₂, —NH₂, —NHC₁-C₃alkyl, —N(C₁-C₃alkyl)₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, C₁-C₃alkyl, C₁-C₃haloalkyl, C₁-C₃deuteroalkyl, C₁-C₃hydroxyalkyl, C₁-C₃aminoalkyl, C₁-C₃heteroalkyl, C₃-C₆cycloalkyl, or heterocycloalkyl;
or two R on the same atom are taken together to form an oxo.

In some embodiments of a compound of Formula (I), the compound is of Formula (Ia):

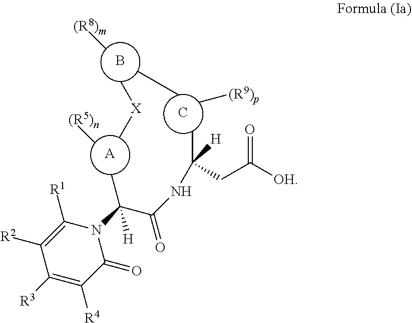

In some embodiments of a compound of Formula (I), the compound is of Formula (Ib):

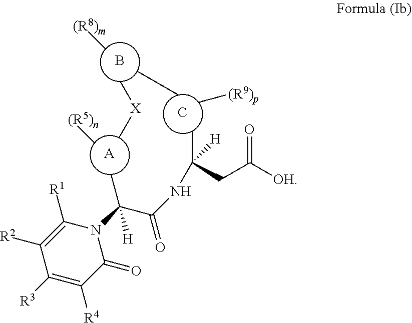

In some embodiments of a compound of Formula (I), the compound is of Formula (Ic):

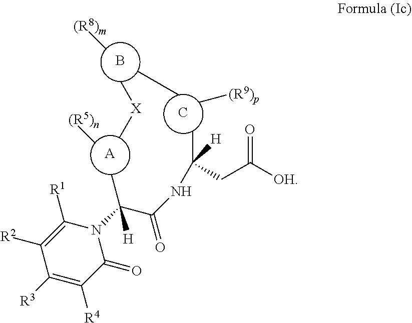

In some embodiments of a compound of Formula (I), the compound is of Formula (Id):

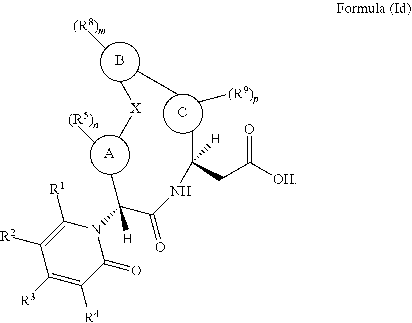

Also disclosed herein is a pharmaceutical composition comprising a compound disclosed herein, or a pharmaceutically acceptable salt, solvate, or stereoisomer thereof, and a pharmaceutically acceptable excipient.

Also disclosed herein is a method of treating a disease, the method comprising administering a compound disclosed herein, or a pharmaceutically acceptable salt, solvate, or stereoisomer thereof. In some embodiments, the disease is inflammatory bowel disease, ileoanal anastomosis, eosinophilic esophagitis, pancreatitis, insulin-dependent diabetes mellitus, mastitis, cholecystitis, cholangitis, pericholangitis, chronic bronchitis, chronic sinusitis, asthma, graft versus host disease, or a chronic inflammatory disease of the lung. In some embodiments, the disease is inflammatory bowel disease. In some embodiments, the inflammatory bowel disease is colitis, Crohn's disease, ileitis, Celiac disease, nontropical Sprue, enteropathy associated with seronegative arthropathies, gastroenteritis, or pouchitis. In some embodiments, the disease is colitis. In some embodiments, the colitis is ulcerative colitis, microscopic colitis, or collagenous colitis. In some embodiments, the disease is pouchitis; and the pouchitis is the result of proctocolectomy. In some embodiments, the disease is gastroenteritis. In some embodiments, the gastroenteritis is eosinophilic gastroenteritis. In some embodiments, the disease is eosinophilic esophagitis. In some embodiments, the disease is a chronic inflammatory disease of the lung. In some embodiments, the chronic inflammatory disease of the lung is interstitial fibrosis. In some embodiments, the interstitial fibrosis is hypersensitivity pneumonitis, collagen diseases, or sarcoidosis.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION

### Definitions

In the following description, certain specific details are set forth in order to provide a thorough understanding of various embodiments. However, one skilled in the art will understand that the subject matter of the disclosure may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed subject matter.

Reference throughout this specification to “some embodiments” or “an embodiment” means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The terms below, as used herein, have the following meanings, unless indicated otherwise:

“oxo” refers to ═O.

“Carboxyl” refers to —COOH.

“Cyano” refers to —CN.

“Alkyl” refers to a straight-chain or branched-chain saturated hydrocarbon monoradical having from one to about ten carbon atoms, or one to six carbon atoms. Examples include, but are not limited to methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl and hexyl, and longer alkyl groups, such as heptyl, octyl and the like. Whenever it appears herein, a numerical range such as “C₁-C₆ alkyl” or “C₁₋₆alkyl”, means that the alkyl group may consist of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated. In some embodiments, the alkyl is a C₁₋₁₀alkyl. In some embodiments, the alkyl is a C₁₋₆alkyl. In some embodiments, the alkyl is a C₁₋₅alkyl. In some embodiments, the alkyl is a C₁₋₄alkyl. In some embodiments, the alkyl is a C₁₋₃alkyl. Unless stated otherwise specifically in the specification, an alkyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkyl is optionally substituted with oxo, halogen, —CN, —COOH, —COOMe, —OH, —OMe, —NH₂, or —NO₂. In some embodiments, the alkyl is optionally substituted with halogen, —CN, —OH, or —OMe. In some embodiments, the alkyl is optionally substituted with halogen.

“Alkenyl” refers to a straight-chain or branched-chain hydrocarbon monoradical having one or more carbon-carbon double-bonds and having from two to about ten carbon atoms, or two to about six carbon atoms. The group may be in either the cis or trans conformation about the double bond(s) and should be understood to include both isomers. Examples include, but are not limited to ethenyl (—CH═CH₂), 1-propenyl (—CH₂CH═CH₂), isopropenyl [—C(CH₃)═CH₂], butenyl, 1,3-butadienyl and the like. Whenever it appears herein, a numerical range such as “C₂-C₆ alkenyl” or “C₂₋₆alkenyl”, means that the alkenyl group may consist of 2 carbon atoms, 3 carbon atoms, 4 carbon atoms, 5 carbon atoms or 6 carbon atoms, although the present definition also covers the occurrence of the term “alkenyl” where no numerical range is designated. Unless stated otherwise specifically in the specification, an alkenyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkenyl is optionally substituted with oxo, halogen, —CN, —COOH, —COOMe, —OH, —OMe, —NH₂, or —NO₂. In some embodiments, the alkenyl is optionally substituted with halogen, —CN, —OH, or —OMe. In some embodiments, the alkenyl is optionally substituted with halogen.

“Alkynyl” refers to a straight-chain or branched-chain hydrocarbon monoradical having one or more carbon-carbon triple-bonds and having from two to about ten carbon atoms, or from two to about six carbon atoms. Examples include, but are not limited to ethynyl, 2-propynyl, 2-butynyl, 1,3-butadiynyl and the like. Whenever it appears herein, a numerical range such as “C₂-C₆ alkynyl” or “C₂₋₆alkynyl”, means that the alkynyl group may consist of 2 carbon atoms, 3 carbon atoms, 4 carbon atoms, 5 carbon atoms or 6 carbon atoms, although the present definition also covers the occurrence of the term “alkynyl” where no numerical range is designated. Unless stated otherwise specifically in the specification, an alkynyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkynyl is optionally substituted with oxo, halogen, —CN, —COOH, COOMe, —OH, —OMe, —NH₂, or —NO₂. In some embodiments, the alkynyl is optionally substituted with halogen, —CN, —OH, or —OMe. In some embodiments, the alkynyl is optionally substituted with halogen.

“Alkylene” refers to a straight or branched divalent hydrocarbon chain. Unless stated otherwise specifically in the specification, an alkylene group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkylene is optionally substituted with oxo, halogen, —CN, —COOH, COOMe, —OH, —OMe, —NH₂, or —NO₂. In some embodiments, the alkylene is optionally substituted with halogen, —CN, —OH, or —OMe. In some embodiments, the alkylene is optionally substituted with halogen.

“Alkoxy” refers to a radical of the formula —OR_{g} where R_{g} is an alkyl radical as defined. Unless stated otherwise specifically in the specification, an alkoxy group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkoxy is optionally substituted with halogen, —CN, —COOH, COOMe, —OH, —OMe, —NH₂, or —NO₂. In some embodiments, the alkoxy is optionally substituted with halogen, —CN, —OH, or —OMe. In some embodiments, the alkoxy is optionally substituted with halogen.

“Amino” refers to the group —NR^{y}R^{z} wherein R^{y} and R^{z} are independently hydrogen, alkyl, alkenyl, alkynyl, cycloalkyl, heterocyclyl, aryl, heteroalkyl, or heteroaryl; each of which may be optionally substituted, as defined herein.

“Aryl” refers to a radical derived from a hydrocarbon ring system comprising 6 to 30 carbon atoms and at least one aromatic ring. The aryl radical may be a monocyclic, bicyclic, tricyclic, or tetracyclic ring system, which may include fused (when fused with a cycloalkyl or heterocycloalkyl ring, the aryl is bonded through an aromatic ring atom) or bridged ring systems. In some embodiments, the aryl is a 6- to 10-membered aryl. In some embodiments, the aryl is a 6-membered aryl (phenyl). Aryl radicals include, but are not limited to, aryl radicals derived from the hydrocarbon ring systems of anthrylene, naphthylene, phenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, an aryl may be optionally substituted, for example, with halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the aryl is optionally substituted with halogen, methyl, ethyl, —CN, —COOH, COOMe, —CF₃, —OH, —OMe, —NH₂, or —NO₂. In some embodiments, the aryl is optionally substituted with halogen, methyl, ethyl, —CN, —CF₃, —OH, or —OMe. In some embodiments, the aryl is optionally substituted with halogen.

“Carboxylate” refers to —C(O)OR^{x}, wherein R^{x} is alkyl, alkenyl, alkynyl, cycloalkyl, heterocyclyl, aryl, heteroalkyl, or heteroaryl; each of which may be optionally substituted, as defined herein.

“Cycloalkyl” refers to a partially or fully saturated, monocyclic, or polycyclic carbocyclic ring, which may include fused (when fused with an aryl or a heteroaryl ring, the cycloalkyl is bonded through a non-aromatic ring atom) or bridged ring systems. In some embodiments, the cycloalkyl is fully saturated. Representative cycloalkyls include, but are not limited to, cycloalkyls having from three to fifteen carbon atoms (C₃-C₁₅ fully saturated cycloalkyl or C₃-C₁₅ cycloalkenyl), from three to ten carbon atoms (C₃-C₁₀ fully saturated cycloalkyl or C₃-C₁₀ cycloalkenyl), from three to eight carbon atoms (C₃-C₈ fully saturated cycloalkyl or C₃-C₈ cycloalkenyl), from three to six carbon atoms (C₃-C₆ fully saturated cycloalkyl or C₃-C₆ cycloalkenyl), from three to five carbon atoms (C₃-C₅ fully saturated cycloalkyl or C₃-C₈ cycloalkenyl), or three to four carbon atoms (C₃-C₄ fully saturated cycloalkyl or C₃-C₄ cycloalkenyl). In some embodiments, the cycloalkyl is a 3- to 10-membered fully saturated cycloalkyl or a 3- to 10-membered cycloalkenyl. In some embodiments, the cycloalkyl is a 3- to 6-membered fully saturated cycloalkyl or a 3- to 6-membered cycloalkenyl. In some embodiments, the cycloalkyl is a 5- to 6-membered fully saturated cycloalkyl or a 5- to 6-membered cycloalkenyl. Monocyclic cycloalkyls include, for example, cyclopropyl, cyclobutyl, cyclopentyl, cyclohexyl, cycloheptyl, and cyclooctyl. Polycyclic cycloalkyls include, for example, adamantyl, norbornyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl. Partially saturated cycloalkyls include, for example cyclopentenyl, cyclohexenyl, cycloheptenyl, and cyclooctenyl. Unless stated otherwise specifically in the specification, a cycloalkyl is optionally substituted, for example, with oxo, halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, a cycloalkyl is optionally substituted with oxo, halogen, methyl, ethyl, —CN, —COOH, COOMe, —CF₃, —OH, —OMe, —NH₂, or —NO₂. In some embodiments, a cycloalkyl is optionally substituted with oxo, halogen, methyl, ethyl, —CN, —CF₃, —OH, or —OMe. In some embodiments, the cycloalkyl is optionally substituted with halogen.

“Halo” or “halogen” refers to bromo, chloro, fluoro or iodo. In some embodiments, halogen is fluoro or chloro. In some embodiments, halogen is fluoro.

“Haloalkyl” refers to an alkyl radical, as defined above, that is substituted by one or more halo radicals, as defined above, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like.

“Hydroxyalkyl” refers to an alkyl radical, as defined above, that is substituted by one or more hydroxyls. In some embodiments, the alkyl is substituted with one hydroxyl. In some embodiments, the alkyl is substituted with one, two, or three hydroxyls. Hydroxyalkyl include, for example, hydroxymethyl, hydroxyethyl, hydroxypropyl, hydroxybutyl, or hydroxypentyl. In some embodiments, the hydroxyalkyl is hydroxymethyl.

“Aminoalkyl” refers to an alkyl radical, as defined above, that is substituted by one or more amines. In some embodiments, the alkyl is substituted with one amine. In some embodiments, the alkyl is substituted with one, two, or three amines. Aminoalkyl include, for example, aminomethyl, aminoethyl, aminopropyl, aminobutyl, or aminopentyl. In some embodiments, the aminoalkyl is aminomethyl.

“Deuteroalkyl” refers to an alkyl radical, as defined above, that is substituted by one or more deuteriums. In some embodiments, the alkyl is substituted with one deuterium. In some embodiments, the alkyl is substituted with one, two, or three deuteriums. In some embodiments, the alkyl is substituted with one, two, three, four, five, or six deuteriums. Deuteroalkyl include, for example, CD₃, CH₂D, CHD₂, CH₂CD₃, CD₂CD₃, CHDCD₃, CH₂CH₂D, or CH₂CHD₂. In some embodiments, the deuteroalkyl is CD₃.

“Heteroalkyl” refers to an alkyl group in which one or more skeletal atoms of the alkyl are selected from an atom other than carbon, e.g., oxygen, nitrogen (e.g., —NH—, —N(alkyl)-), sulfur, phosphorus, or combinations thereof. A heteroalkyl is attached to the rest of the molecule at a carbon atom of the heteroalkyl. In one aspect, a heteroalkyl is a C₁-C₆ heteroalkyl wherein the heteroalkyl is comprised of 1 to 6 carbon atoms and one or more atoms other than carbon, e.g., oxygen, nitrogen (e.g. —NH—, —N(alkyl)-), sulfur, phosphorus, or combinations thereof wherein the heteroalkyl is attached to the rest of the molecule at a carbon atom of the heteroalkyl. Examples of such heteroalkyl are, for example, —CH₂OCH₃, —CH₂CH₂OCH₃, —CH₂CH₂OCH₂CH₂OCH₃, —CH(CH₃)OCH₃, —CH₂NHCH₃, —CH₂N(CH₃)₂, —CH₂CH₂NHCH₃, or —CH₂CH₂N(CH₃)₂. Unless stated otherwise specifically in the specification, a heteroalkyl is optionally substituted for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroalkyl is optionally substituted with oxo, halogen, methyl, ethyl, —CN, —CF₃, —OH, —OMe, —NH₂, or —NO₂. In some embodiments, a heteroalkyl is optionally substituted with oxo, halogen, methyl, ethyl, —CN, —CF₃, —OH, or —OMe. In some embodiments, the heteroalkyl is optionally substituted with halogen.

“Heterocycloalkyl” refers to a 3- to 24-membered partially or fully saturated ring radical comprising 2 to 23 carbon atoms and from one to 8 heteroatoms selected from the group consisting of nitrogen, oxygen, phosphorous and sulfur. In some embodiments, the heterocycloalkyl is fully saturated. In some embodiments, the heterocycloalkyl comprises one to three heteroatoms selected from the group consisting of nitrogen, oxygen, and sulfur. In some embodiments, the heterocycloalkyl comprises one to three heteroatoms selected from the group consisting of nitrogen and oxygen. In some embodiments, the heterocycloalkyl comprises one to three nitrogens. In some embodiments, the heterocycloalkyl comprises one or two nitrogens. In some embodiments, the heterocycloalkyl comprises one nitrogen. In some embodiments, the heterocycloalkyl comprises one nitrogen and one oxygen. Unless stated otherwise specifically in the specification, the heterocycloalkyl radical may be a monocyclic, bicyclic, tricyclic, or tetracyclic ring system, which may include fused (when fused with an aryl or a heteroaryl ring, the heterocycloalkyl is bonded through a non-aromatic ring atom) or bridged ring systems; and the nitrogen, carbon, or sulfur atoms in the heterocycloalkyl radical may be optionally oxidized; the nitrogen atom may be optionally quaternized. Representative heterocycloalkyls include, but are not limited to, heterocycloalkyls having from two to fifteen carbon atoms (C₂-C₁₅ fully saturated heterocycloalkyl or C₂-C₁₅ heterocycloalkenyl), from two to ten carbon atoms (C₂-C₁₀ fully saturated heterocycloalkyl or C₂-C₁₀ heterocycloalkenyl), from two to eight carbon atoms (C₂-C₈ fully saturated heterocycloalkyl or C₂-C₈ heterocycloalkenyl), from two to seven carbon atoms (C₂-C₇ fully saturated heterocycloalkyl or C₂-C₇ heterocycloalkenyl), from two to six carbon atoms (C₂-C₆ fully saturated heterocycloalkyl or C₂-C₆ heterocycloalkenyl), from two to five carbon atoms (C₂-C₅ fully saturated heterocycloalkyl or C₂-C₅ heterocycloalkenyl), or two to four carbon atoms (C₂-C₄ fully saturated heterocycloalkyl or C₂-C₄ heterocycloalkenyl). Examples of such heterocycloalkyl radicals include, but are not limited to, aziridinyl, azetidinyl, oxetanyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 1,3-dihydroisobenzofuran-1-yl, 3-oxo-1,3-dihydroisobenzofuran-1-yl, methyl-2-oxo-1,3-dioxol-4-yl, and 2-oxo-1,3-dioxol-4-yl. The term heterocycloalkyl also includes all ring forms of the carbohydrates, including but not limited to the monosaccharides, the disaccharides, and the oligosaccharides. Unless otherwise noted, heterocycloalkyls have from 2 to 10 carbons in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). In some embodiments, the heterocycloalkyl is a 3- to 8-membered heterocycloalkyl. In some embodiments, the heterocycloalkyl is a 3- to 7-membered heterocycloalkyl. In some embodiments, the heterocycloalkyl is a 3- to 6-membered heterocycloalkyl. In some embodiments, the heterocycloalkyl is a 4- to 6-membered heterocycloalkyl. In some embodiments, the heterocycloalkyl is a 5- to 6-membered heterocycloalkyl. In some embodiments, the heterocycloalkyl is a 3- to 8-membered heterocycloalkenyl. In some embodiments, the heterocycloalkyl is a 3- to 7-membered heterocycloalkenyl. In some embodiments, the heterocycloalkyl is a 3- to 6-membered heterocycloalkenyl. In some embodiments, the heterocycloalkyl is a 4- to 6-membered heterocycloalkenyl. In some embodiments, the heterocycloalkyl is a 5- to 6-membered heterocycloalkenyl. Unless stated otherwise specifically in the specification, a heterocycloalkyl may be optionally substituted as described below, for example, with oxo, halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the heterocycloalkyl is optionally substituted with oxo, halogen, methyl, ethyl, —CN, —COOH, COOMe, —CF₃, —OH, —OMe, —NH₂, or —NO₂. In some embodiments, the heterocycloalkyl is optionally substituted with halogen, methyl, ethyl, —CN, —CF₃, —OH, or —OMe. In some embodiments, the heterocycloalkyl is optionally substituted with halogen.

“Heteroaryl” refers to a 5- to 14-membered ring system radical comprising one to thirteen carbon atoms, one to six heteroatoms selected from the group consisting of nitrogen, oxygen, phosphorous, and sulfur, and at least one aromatic ring. In some embodiments, the heteroaryl comprises one to three heteroatoms selected from the group consisting of nitrogen, oxygen, and sulfur. In some embodiments, the heteroaryl comprises one to three heteroatoms selected from the group consisting of nitrogen and oxygen. In some embodiments, the heteroaryl comprises one to three nitrogens. In some embodiments, the heteroaryl comprises one or two nitrogens. In some embodiments, the heteroaryl comprises one nitrogen. The heteroaryl radical may be a monocyclic, bicyclic, tricyclic, or tetracyclic ring system, which may include fused (when fused with a cycloalkyl or heterocycloalkyl ring, the heteroaryl is bonded through an aromatic ring atom) or bridged ring systems; and the nitrogen, carbon, or sulfur atoms in the heteroaryl radical may be optionally oxidized; the nitrogen atom may be optionally quaternized. In some embodiments, the heteroaryl is a 5- to 10-membered heteroaryl. In some embodiments, the heteroaryl is a 5- to 6-membered heteroaryl. In some embodiments, the heteroaryl is a 6-membered heteroaryl. In some embodiments, the heteroaryl is a 5-membered heteroaryl. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl may be optionally substituted, for example, with halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the heteroaryl is optionally substituted with halogen, methyl, ethyl, —CN, —COOH, COOMe, —CF₃, —OH, —OMe, —NH₂, or —NO₂. In some embodiments, the heteroaryl is optionally substituted with halogen, methyl, ethyl, —CN, —CF₃, —OH, or —OMe. In some embodiments, the heteroaryl is optionally substituted with halogen.

The term “optional” or “optionally” means that the subsequently described event or circumstance may or may not occur, and that the description includes instances where said event or circumstance occurs and instances in which it does not. For example, “optionally substituted alkyl” means either “alkyl” or “substituted alkyl” as defined above. Further, an optionally substituted group may be un-substituted (e.g., —CH₂CH₃), fully substituted (e.g., —CF₂CF₃), mono-substituted (e.g., —CH₂CH₂F) or substituted at a level anywhere in-between fully substituted and mono-substituted (e.g., —CH₂CHF₂, —CH₂CF₃, —CF₂CH₃, —CFHCHF₂, etc.). It will be understood by those skilled in the art with respect to any group containing one or more substituents that such groups are not intended to introduce any substitution or substitution patterns (e.g., substituted alkyl includes optionally substituted cycloalkyl groups, which in turn are defined as including optionally substituted alkyl groups, potentially ad infinitum) that are sterically impractical and/or synthetically non-feasible. Thus, any substituents described should generally be understood as having a maximum molecular weight of about 1,000 daltons, and more typically, up to about 500 daltons.

The term “one or more” when referring to an optional substituent means that the subject group is optionally substituted with one, two, three, four, or more substituents. In some embodiments, the subject group is optionally substituted with one, two, three, or four substituents. In some embodiments, the subject group is optionally substituted with one, two, or three substituents. In some embodiments, the subject group is optionally substituted with one or two substituents. In some embodiments, the subject group is optionally substituted with one substituent. In some embodiments, the subject group is optionally substituted with two substituents.

An “effective amount” or “therapeutically effective amount” refers to an amount of a compound administered to a mammalian subject, either as a single dose or as part of a series of doses, which is effective to produce a desired therapeutic effect.

“Treatment” of an individual (e.g., a mammal, such as a human) or a cell is any type of intervention used in an attempt to alter the natural course of the individual or cell. In some embodiments, treatment includes administration of a pharmaceutical composition, subsequent to the initiation of a pathologic event or contact with an etiologic agent and includes stabilization of the condition (e.g., condition does not worsen) or alleviation of the condition.

“Synergy” or “synergize” refers to an effect of a combination that is greater than additive of the effects of each component alone at the same doses.

As used herein, a “disease or disorder associated with α₄β₇ integrin” or, alternatively, “an α₄β₇ integrin-mediated disease or disorder” means any disease or other deleterious condition in which α₄β₇ integrin, or a mutant thereof, is known or suspected to play a role.

The terms “α₄β₇,” “a4B7,” “a4b7,” “alpha-4 beta-7,” and “alpha 4 beta 7” and the like as used herein all refer to α₄β₇.

### Compounds

Described herein are compounds, or a pharmaceutically acceptable salt, solvate, or stereoisomer thereof useful in the treatment of cancer.

Disclosed herein is a compound of Formula (I), or a pharmaceutically acceptable salt, solvate, or stereoisomer thereof:

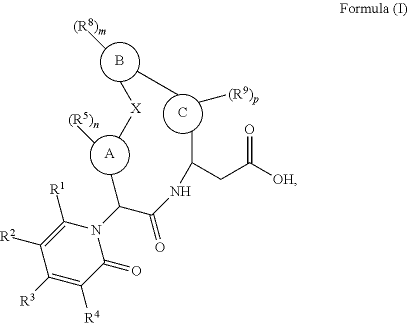

wherein:
R¹ is hydrogen, deuterium, halogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl; wherein the alkyl, cycloalkyl, and heterocycloalkyl is optionally substituted with one or more R;
R² is hydrogen, deuterium, halogen, -L-CN, -L-OH, -L-OR^{a}, -L-S(═O)R^{a}, -L-S(═O)₂R^{a}, -L-S(═O)₂NR^{c}R^{d}, -L-NR^{c}R^{d}, -L-C(═O)R^{a}, -L-C(═O)OR^{b}, -L-C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, -L-cycloalkyl, or -L-heterocycloalkyl; wherein the alkyl, alkenyl, alkynyl, cycloalkyl, and heterocycloalkyl is optionally substituted with one or more R;
R³ is hydrogen, deuterium, halogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl; wherein the alkyl, cycloalkyl, and heterocycloalkyl is optionally substituted with one or more R;
R⁴ is hydrogen, deuterium, halogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl; wherein the alkyl, cycloalkyl, and heterocycloalkyl is optionally substituted with one or more R;
L is absent or C₁-C₆alkylene;
Ring A is aryl or heteroaryl;
each R⁵ is independently halogen, —CN, —NO₂, —OH, —OR^{a}, —SH, —SR^{a}, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or two R are taken together to form an oxo;
n is 0-4;
X is —O—, —S—, —C(R⁶)₂—, —NR—, —[C(R⁶)₂]₂—, —O—C(R⁶)₂—, —C(R⁶)₂—O—, —S—C(R⁶)₂—, or —C(R⁶)₂—S—;
each R⁶ is independently hydrogen, deuterium, halogen, —OH, —OR^{a}, C₁-C₆alkyl, C₁-C₆haloalkyl, C₁-C₆hydroxyalkyl, C₁-C₆aminoalkyl, or C₁-C₆heteroalkyl;
or two R⁶ are taken together to form a cycloalkyl or heterocycloalkyl; each optionally substituted with one or more R;
R is hydrogen, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl;
Ring B is aryl or heteroaryl;
each R⁸ is independently halogen, —CN, —NO₂, —OH, —OR^{a}, —SH, —SR^{a}, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or two R⁸ are taken together to form an oxo;
m is 0-4;
Ring C is aryl or heteroaryl;
each R⁹ is independently halogen, —CN, —NO₂, —OH, —OR^{a}, —SH, —SR^{a}, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or two R⁹ are taken together to form an oxo;
p is 0-4;
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each alkyl, alkylene, alkenyl, alkynyl, cycloalkyl, heterocycloalkyl, aryl, and heteroaryl is independently optionally substituted with one or more R;
each R is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each alkyl, alkylene, alkenyl, alkynyl, cycloalkyl, heterocycloalkyl, aryl, and heteroaryl is independently optionally substituted with one or more R;
R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each alkyl, alkylene, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R; and
each R is independently halogen, —CN, —OH, —OC₁-C₃alkyl, —OC₁-C₃haloalkyl, —SC₁-C₃alkyl, —S(═O)C₁-C₃alkyl, —S(═O)₂C₁-C₃alkyl, —S(═O)₂NH₂, —S(═O)₂NHC₁-C₃alkyl, —S(═O)₂N(C₁-C₃alkyl)₂, —NH₂, —NHC₁-C₃alkyl, —N(C₁-C₃alkyl)₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, C₁-C₃alkyl, C₁-C₃haloalkyl, C₁-C₃deuteroalkyl, C₁-C₃hydroxyalkyl, C₁-C₃aminoalkyl, C₁-C₃heteroalkyl, C₃-C₆cycloalkyl, or heterocycloalkyl;
or two R on the same atom are taken together to form an oxo.

In some embodiments of a compound of Formula (I), the compound is of Formula (Ia):

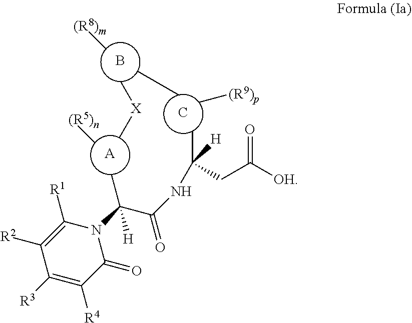

In some embodiments of a compound of Formula (I), the compound is of Formula (Ib):

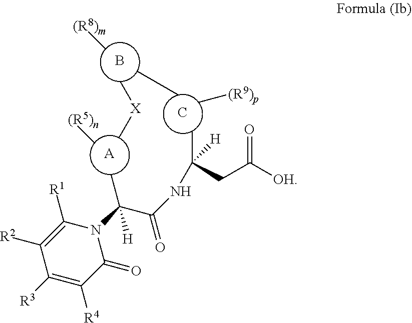

In some embodiments of a compound of Formula (I), the compound is of Formula (Ic):

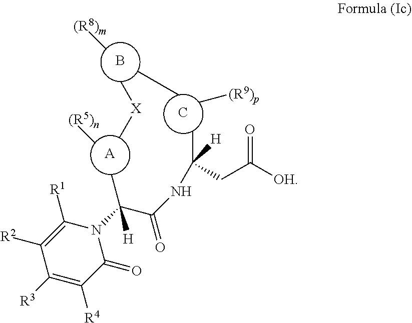

In some embodiments of a compound of Formula (I), the compound is of Formula (Id):

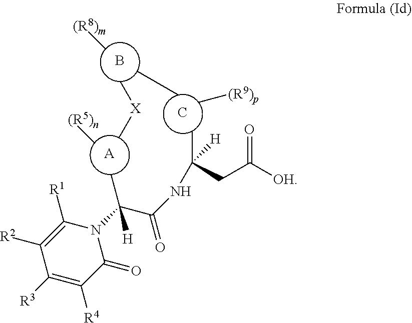

In some embodiments of a compound of Formula (I) or (Ia)-(Id), Ring A is aryl. In some embodiments of a compound of Formula (I) or (Ia)-(Id), Ring A is phenyl. In some embodiments of a compound of Formula (I) or (Ia)-(Id), Ring A is heteroaryl. In some embodiments of a compound of Formula (I) or (Ia)-(Id), Ring A is 5- or 6-membered heteroaryl. In some embodiments of a compound of Formula (I) or (Ia)-(Id), Ring A is 5-membered heteroaryl. In some embodiments of a compound of Formula (I) or (Ia)-(Id), Ring A is 6-membered heteroaryl.

In some embodiments of a compound of Formula (I), the compound is of Formula (II):

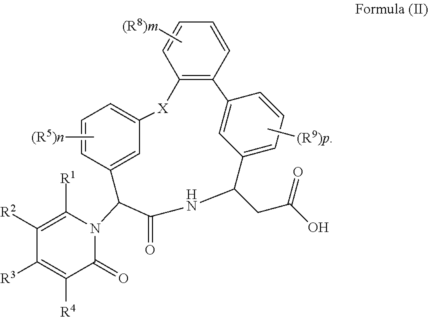

In some embodiments of a compound of Formula (I), the compound is of Formula (IIa):

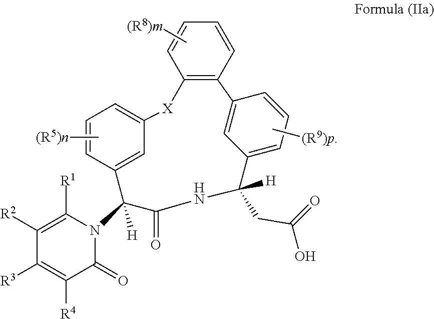

In some embodiments of a compound of Formula (I), the compound is of Formula (IIb):

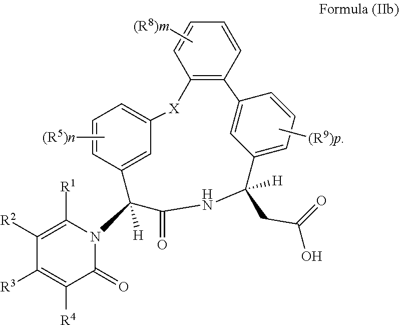

In some embodiments of a compound of Formula (I), the compound is of Formula (IIc):

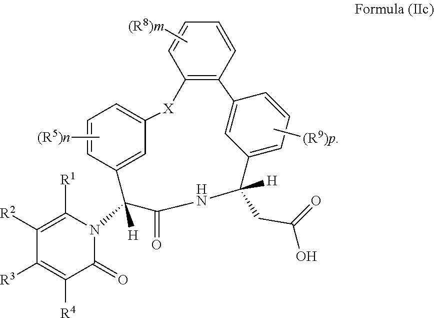

In some embodiments of a compound of Formula (I), the compound is of Formula (IId):

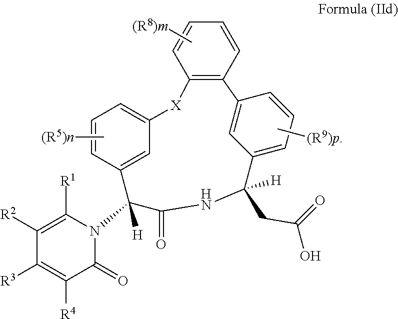

In some embodiments of a compound of Formula (I) or (Ia)-(Id), each R is independently halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl; wherein each alkyl, cycloalkyl, and heterocycloalkyl is independently optionally substituted with one or more R.

In some embodiments of a compound of Formula (I) or (Ia)-(Id), each R is independently halogen, C₁-C₆alkyl, C₁-C₆haloalkyl, cycloalkyl, or heterocycloalkyl; wherein each alkyl, cycloalkyl, and heterocycloalkyl is independently optionally substituted with one or more R. In some embodiments of a compound of Formula (I) or (Ia)-(Id), each R is independently halogen, C₁-C₆alkyl, C₁-C₆haloalkyl, or cycloalkyl. In some embodiments of a compound of Formula (I) or (Ia)-(Id), each R⁵ is independently halogen. In some embodiments of a compound of Formula (I) or (Ia)-(Id), each R⁵ is independently C₁-C₆alkyl, C₁-C₆haloalkyl, or cycloalkyl. In some embodiments of a compound of Formula (I) or (Ia)-(Id), each R⁵ is independently C₁-C₆alkyl or C₁-C₆haloalkyl.

In some embodiments of a compound of Formula (I) or (Ia)-(Id), n is 0-2. In some embodiments of a compound of Formula (I) or (Ia)-(Id), n is 1 or 2. In some embodiments of a compound of Formula (I) or (Ia)-(Id), n is 0 or 1. In some embodiments of a compound of Formula (I) or (Ia)-(Id), n is 0. In some embodiments of a compound of Formula (I) or (Ia)-(Id), n is 1. In some embodiments of a compound of Formula (I) or (Ia)-(Id), n is 2. In some embodiments of a compound of Formula (I) or (Ia)-(Id), n is 3.

In some embodiments of a compound of Formula (I) or (Ia)-(Id), X is —O—, —S—, —C(R⁶)₂—, or —NR⁷—. In some embodiments of a compound of Formula (I) or (Ia)-(Id), X is —O—, —S—, or —NR⁷—. In some embodiments of a compound of Formula (I) or (Ia)-(Id), X is —O—. In some embodiments of a compound of Formula (I) or (Ia)-(Id), X is —S—. In some embodiments of a compound of Formula (I) or (Ia)-(Id), X is —C(R⁶)₂—. In some embodiments of a compound of Formula (I) or (Ia)-(Id), X is —NR⁷—.

In some embodiments of a compound of Formula (I), the compound is of Formula (Ie):

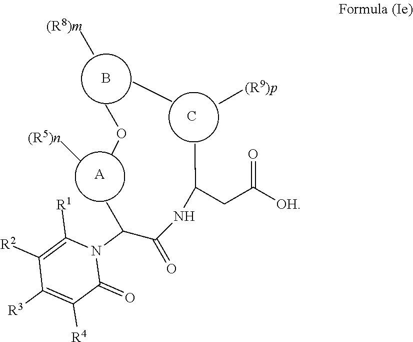

In some embodiments of a compound of Formula (I), the compound is of Formula (IIe):

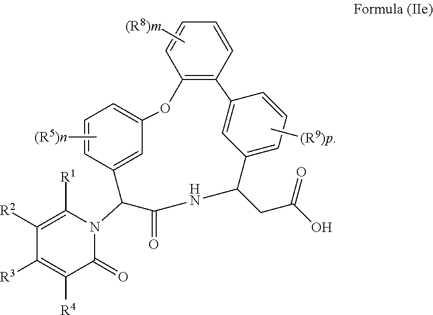

In some embodiments of a compound of Formula (I) or (Ia)-(Id), each R⁶ is independently hydrogen, deuterium, halogen, C₁-C₆alkyl, or C₁-C₆haloalkyl. In some embodiments of a compound of Formula (I) or (Ia)-(Id), each R⁶ is independently hydrogen, halogen, or C₁-C₆alkyl. In some embodiments of a compound of Formula (I) or (Ia)-(Id), each R⁶ is hydrogen.

In some embodiments of a compound of Formula (I) or (Ia)-(Id), two R⁶ are taken together to form a cycloalkyl or heterocycloalkyl; each optionally substituted with one or more R.

In some embodiments of a compound of Formula (I), the compound is of Formula (If):

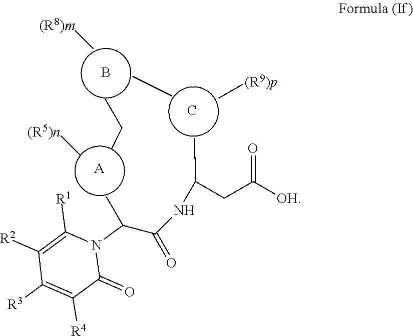

In some embodiments of a compound of Formula (I), the compound is of Formula (Ig):

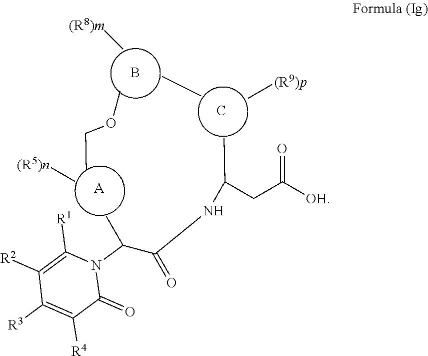

In some embodiments of a compound of Formula (I), the compound is of Formula (IIf):

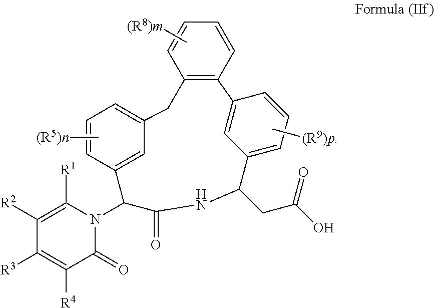

In some embodiments of a compound of Formula (I), the compound is of Formula (IIg):

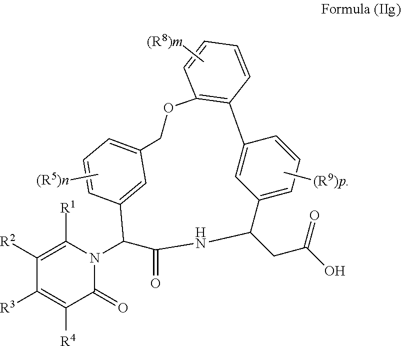

In some embodiments of a compound of Formula (I) or (Ia)-(Id), R⁷ is hydrogen, C₁-C₆alkyl, or C₁-C₆haloalkyl. In some embodiments of a compound of Formula (I) or (Ia)-(Id), R is hydrogen.

In some embodiments of a compound of Formula (I) or (Ia)-(Id), Ring B is aryl. In some embodiments of a compound of Formula (I) or (Ia)-(Id), Ring B is phenyl. In some embodiments of a compound of Formula (I) or (Ia)-(Id), Ring B is heteroaryl. In some embodiments of a compound of Formula (I) or (Ia)-(Id), Ring B is 5- or 6-membered heteroaryl. In some embodiments of a compound of Formula (I) or (Ia)-(Id), Ring B is 5-membered heteroaryl. In some embodiments of a compound of Formula (I) or (Ia)-(Id), Ring B is 6-membered heteroaryl.

In some embodiments of a compound of Formula (I) or (Ia)-(Id), each R⁸ is independently halogen, —CN, —NO₂, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl; wherein each alkyl, cycloalkyl, and heterocycloalkyl is independently optionally substituted with one or more R.

In some embodiments of a compound of Formula (I) or (Ia)-(Id), each R⁸ is independently halogen, C₁-C₆alkyl, or C₁-C₆haloalkyl.

In some embodiments of a compound of Formula (I) or (Ia)-(Id), m is 0-2. In some embodiments of a compound of Formula (I) or (Ia)-(Id), m is 1 or 2. In some embodiments of a compound of Formula (I) or (Ia)-(Id), m is 0 or 1. In some embodiments of a compound of Formula (I) or (Ia)-(Id), m is 0. In some embodiments of a compound of Formula (I) or (Ia)-(Id), m is 1. In some embodiments of a compound of Formula (I) or (Ia)-(Id), m is 2. In some embodiments of a compound of Formula (I) or (Ia)-(Id), m is 3.

In some embodiments of a compound of Formula (I) or (Ia)-(Id), Ring C is aryl. In some embodiments of a compound of Formula (I) or (Ia)-(Id), Ring C is phenyl. In some embodiments of a compound of Formula (I) or (Ia)-(Id), Ring C is heteroaryl. In some embodiments of a compound of Formula (I) or (Ia)-(Id), Ring C is 5- or 6-membered heteroaryl. In some embodiments of a compound of Formula (I) or (Ia)-(Id), Ring C is 5-membered heteroaryl.

In some embodiments of a compound of Formula (I) or (Ia)-(Id), each R⁹ is independently halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl; wherein each alkyl, cycloalkyl, and heterocycloalkyl is independently optionally substituted with one or more R. In some embodiments of a compound of Formula (I) or (Ia)-(Id), each R⁹ is independently halogen, —CN, —OH, —OR^{a}, —NR^{c}R^{d}, C₁-C₆alkyl, or C₁-C₆haloalkyl. In some embodiments of a compound of Formula (I) or (Ia)-(Id), each R⁹ is independently halogen, C₁-C₆alkyl, or C₁-C₆haloalkyl.

In some embodiments of a compound of Formula (I) or (Ia)-(Id), p is 0-2. In some embodiments of a compound of Formula (I) or (Ia)-(Id), p is 1 or 2. In some embodiments of a compound of Formula (I) or (Ia)-(Id), p is 0 or 1. In some embodiments of a compound of Formula (I) or (Ia)-(Id), p is 0. In some embodiments of a compound of Formula (I) or (Ia)-(Id), p is 1. In some embodiments of a compound of Formula (I) or (Ia)-(Id), p is 2. In some embodiments of a compound of Formula (I) or (Ia)-(Id), p is 3.

In some embodiments of a compound of Formula (I) or (Ia)-(Id), R¹ is hydrogen, deuterium, halogen, C₁-C₆alkyl, or C₁-C₆haloalkyl; wherein the alkyl is optionally substituted with one or more R. In some embodiments of a compound of Formula (I) or (Ia)-(Id), R¹ is hydrogen, halogen, C₁-C₆alkyl, or C₁-C₆haloalkyl. In some embodiments of a compound of Formula (I) or (Ia)-(Id), R¹ is hydrogen, halogen, or C₁-C₆alkyl. In some embodiments of a compound of Formula (I) or (Ia)-(Id), R¹ is hydrogen.

In some embodiments of a compound of Formula (I) or (Ia)-(Id), R³ is hydrogen, deuterium, halogen, C₁-C₆alkyl, or C₁-C₆haloalkyl; wherein the alkyl is optionally substituted with one or more R. In some embodiments of a compound of Formula (I) or (Ia)-(Id), R³ is hydrogen, halogen, C₁-C₆alkyl, or C₁-C₆haloalkyl. In some embodiments of a compound of Formula (I) or (Ia)-(Id), R³ is hydrogen or C₁-C₆haloalkyl.

In some embodiments of a compound of Formula (I) or (Ia)-(Id), R⁴ is hydrogen, deuterium, halogen, C₁-C₆alkyl, or C₁-C₆haloalkyl; wherein the alkyl is optionally substituted with one or more R. In some embodiments of a compound of Formula (I) or (Ia)-(Id), R⁴ is hydrogen, halogen, C₁-C₆alkyl, or C₁-C₆haloalkyl. In some embodiments of a compound of Formula (I) or (Ia)-(Id), R⁴ is hydrogen or C₁-C₆haloalkyl.

In some embodiments of a compound of Formula (I) or (Ia)-(Id), R² is -L-CN, -L-OH, -L-OR^{a}, -L-NR^{c}R^{d}, C₁-C₆alkyl, C₁-C₆haloalkyl, C₁-C₆hydroxyalkyl, C₁-C₆aminoalkyl, C₁-C₆heteroalkyl, -L-cycloalkyl, or -L-heterocycloalkyl; wherein the alkyl, cycloalkyl, and heterocycloalkyl is optionally substituted with one or more R. In some embodiments of a compound of Formula (I) or (Ia)-(Id), R² is -L-NR^{c}R^{d}, C₁-C₆alkyl, C₁-C₆haloalkyl, C₁-C₆aminoalkyl, -L-cycloalkyl, or -L-heterocycloalkyl; wherein the alkyl, cycloalkyl, and heterocycloalkyl is optionally substituted with one or more R. In some embodiments of a compound of Formula (I) or (Ia)-(Id), R² is -L-NR^{c}R^{d} or -L-heterocycloalkyl; wherein the heterocycloalkyl is optionally substituted with one or more R. In some embodiments of a compound of Formula (I) or (Ia)-(Id), R² is -L-NR^{c}R^{d}. In some embodiments of a compound of Formula (I) or (Ia)-(Id), R² is -L-heterocycloalkyl.

In some embodiments of a compound of Formula (I), the compound is of Formula (Ih):

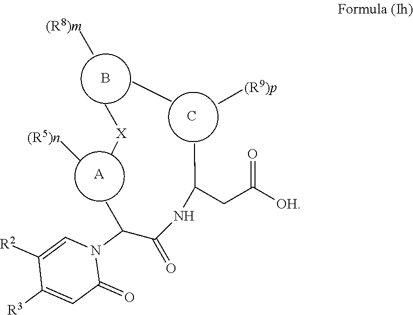

In some embodiments of a compound of Formula (I), the compound is of Formula (Ij):

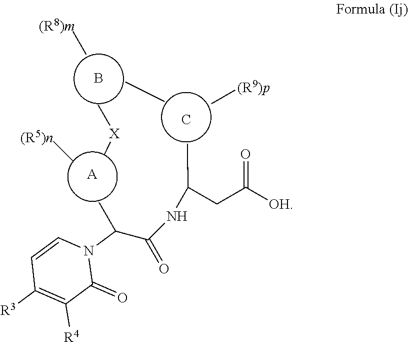

In some embodiments of a compound of Formula (I), the compound is of Formula (IIh):

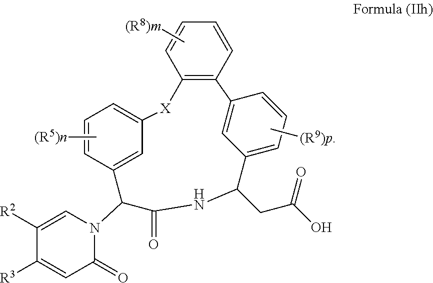

In some embodiments of a compound of Formula (I), the compound is of Formula (IIj):

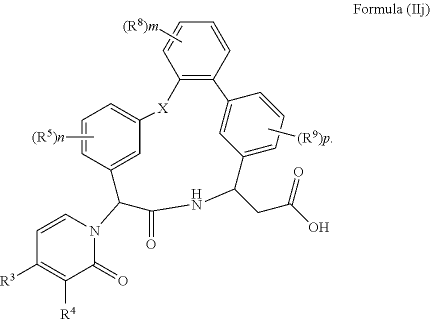

In some embodiments of a compound of Formula (I) or (Ia)-(Id), L is absent. In some embodiments of a compound of Formula (I) or (Ia)-(Id), L is C₁-C₆alkylene. In some embodiments of a compound of Formula (I) or (Ia)-(Id), L is C₁-C₃alkylene. In some embodiments of a compound of Formula (I) or (Ia)-(Id), L is C₁-C₂alkylene. In some embodiments of a compound of Formula (I) or (Ia)-(Id), L is C₁alkylene.

In some embodiments of a compound disclosed herein, each R^{a} is independently C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, C₁-C₆alkylene(cycloalkyl), or C₁-C₆alkylene(heterocycloalkyl), wherein each alkyl, alkylene, cycloalkyl, and heterocycloalkyl is independently optionally substituted with one or more R. In some embodiments of a compound disclosed herein, each R^{a} is independently C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl, wherein each alkyl, cycloalkyl, and heterocycloalkyl is independently optionally substituted with one or more R. In some embodiments of a compound disclosed herein, each R^{a} is independently C₁-C₆alkyl, C₁-C₆haloalkyl, C₁-C₆hydroxyalkyl, C₁-C₆aminoalkyl, or C₁-C₆heteroalkyl, wherein each alkyl is independently optionally substituted with one or more R. In some embodiments of a compound disclosed herein, each R^{a} is independently C₁-C₆alkyl, C₁-C₆haloalkyl, C₁-C₆hydroxyalkyl, C₁-C₆aminoalkyl, or C₁-C₆heteroalkyl. In some embodiments of a compound disclosed herein, each R^{a} is independently C₁-C₆alkyl or C₁-C₆haloalkyl. In some embodiments of a compound disclosed herein, each R^{a} is independently C₁-C₆alkyl.

In some embodiments of a compound disclosed herein, R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, C₁-C₆alkylene(cycloalkyl), or C₁-C₆alkylene(heterocycloalkyl), wherein each alkyl, alkylene, cycloalkyl, and heterocycloalkyl is independently optionally substituted with one or more R. In some embodiments of a compound disclosed herein, R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl, wherein each alkyl, cycloalkyl, and heterocycloalkyl is independently optionally substituted with one or more R. In some embodiments of a compound disclosed herein, R^{c} and R^{d} are each independently hydrogen, C₁-C₆alkyl, C₁-C₆haloalkyl, C₁-C₆hydroxyalkyl, C₁-C₆aminoalkyl, or C₁-C₆heteroalkyl, wherein each alkyl is independently optionally substituted with one or more R. In some embodiments of a compound disclosed herein, R^{c} and R^{d} are each independently hydrogen, C₁-C₆alkyl, C₁-C₆haloalkyl, C₁-C₆hydroxyalkyl, C₁-C₆aminoalkyl, or C₁-C₆heteroalkyl. In some embodiments of a compound disclosed herein, R^{c} and R^{d} are each independently hydrogen, C₁-C₆alkyl, or C₁-C₆haloalkyl. In some embodiments of a compound disclosed herein, R^{c} and R^{d} are each independently hydrogen or C₁-C₆alkyl. In some embodiments of a compound disclosed herein, R^{c} and R^{d} are each hydrogen. In some embodiments of a compound disclosed herein, R^{c} and R^{d} are each independently C₁-C₆alkyl.

In some embodiments of a compound disclosed herein, R^{c} and R^{d} are taken together with the atom to which they are attached to form a heterocycloalkyl optionally substituted with one or more R.

In some embodiments of a compound disclosed herein, each R is independently halogen, —CN, —OH, —OC₁-C₃alkyl, —OC₁-C₃haloalkyl, —NH₂, —NHC₁-C₃alkyl, —N(C₁-C₃alkyl)₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, C₁-C₃alkyl, C₁-C₃haloalkyl, C₁-C₃deuteroalkyl, C₁-C₃hydroxyalkyl, C₁-C₃aminoalkyl, C₁-C₃heteroalkyl, C₃-C₆cycloalkyl, or heterocycloalkyl; or two R on the same atom are taken together to form an oxo. In some embodiments of a compound disclosed herein, each R is independently halogen, —CN, —OH, —OC₁-C₃alkyl, —OC₁-C₃haloalkyl, —NH₂, —NHC₁-C₃alkyl, —N(C₁-C₃alkyl)₂, C₁-C₃alkyl, C₁-C₃haloalkyl, C₁-C₃deuteroalkyl, C₁-C₃hydroxyalkyl, C₁-C₃aminoalkyl, C₁-C₃heteroalkyl, C₃-C₆cycloalkyl, or heterocycloalkyl; or two R on the same atom are taken together to form an oxo. In some embodiments of a compound disclosed herein, each R is independently halogen, —CN, —OH, —OC₁-C₃alkyl, —OC₁-C₃haloalkyl, —NH₂, —NHC₁-C₃alkyl, —N(C₁-C₃alkyl)₂, C₁-C₃alkyl, C₁-C₃haloalkyl, C₃-C₆cycloalkyl, or heterocycloalkyl; or two R on the same atom are taken together to form an oxo. In some embodiments of a compound disclosed herein, each R is independently halogen, —CN, —OH, —OC₁-C₃alkyl, —NH₂, C₁-C₃alkyl, or C₁-C₃haloalkyl; or two R on the same atom are taken together to form an oxo. In some embodiments of a compound disclosed herein, each R is independently halogen, —CN, —OH, or C₁-C₃alkyl; or two R on the same atom are taken together to form an oxo. In some embodiments of a compound disclosed herein, each R is independently halogen or C₁-C₃alkyl; or two R on the same atom are taken together to form an oxo.

Any combination of the groups described above for the various variables is contemplated herein. Throughout the specification, groups and substituents thereof are chosen by one skilled in the field to provide stable moieties and compounds.

In some embodiments, the compound disclosed herein, or a pharmaceutically acceptable salt, solvate, or stereoisomer thereof, is selected from a compound found in Table 1.

TABLE 1

| Ex. | Structure |
|---|---|
| 1 | 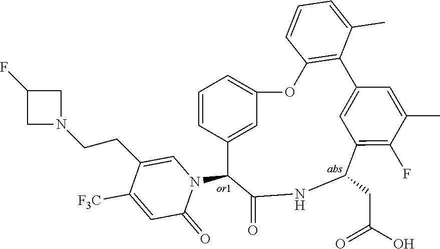 [SMILES: Cc1cc2cc(c1F)[C@H](CC(=O)O)NC(=O)[C@@H](n1cc(CCN3CC(F)C3)c(C(F)(F)F)cc1=O)c1cccc(c1)Oc1cccc(C)c1-2] |
| 2 | 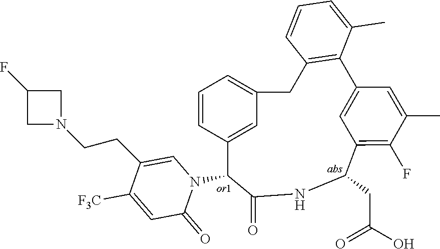 [SMILES: Cc1cc2cc(c1F)[C@H](CC(=O)O)NC(=O)[C@H](n1cc(CCN3CC(F)C3)c(C(F)(F)F)cc1=O)c1cccc(c1)Cc1cccc(C)c1-2] |
| 3 | 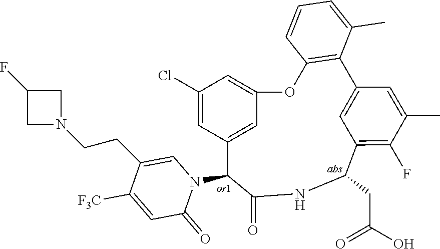 [SMILES: Cc1cc2cc(c1F)[C@H](CC(=O)O)NC(=O)[C@@H](n1cc(CCN3CC(F)C3)c(C(F)(F)F)cc1=O)c1cc(Cl)cc(c1)Oc1cccc(C)c1-2] |
| 4 | 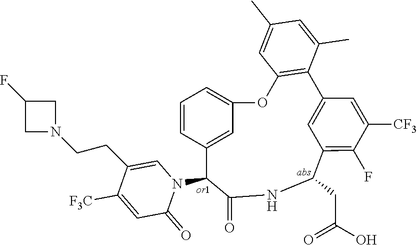 [SMILES: Cc1cc(C)c2c(c1)Oc1cccc(c1)[C@H](n1cc(CCN3CC(F)C3)c(C(F)(F)F)cc1=O)C(=O)N[C@@H](CC(=O)O)c1cc-2cc(C(F)(F)F)c1F] |
| 5 | 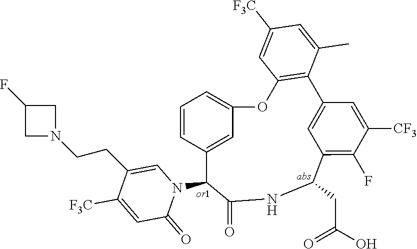 [SMILES: Cc1cc(C(F)(F)F)cc2c1-c1cc(c(F)c(C(F)(F)F)c1)[C@H](CC(=O)O)NC(=O)[C@@H](n1cc(CCN3CC(F)C3)c(C(F)(F)F)cc1=O)c1cccc(c1)O2] |
| 6 | 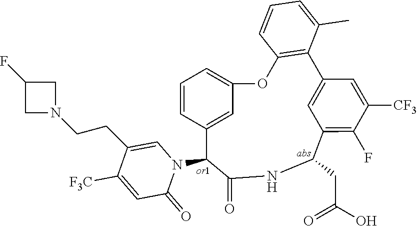 [SMILES: Cc1cccc2c1-c1cc(c(F)c(C(F)(F)F)c1)[C@H](CC(=O)O)NC(=O)[C@@H](n1cc(CCN3CC(F)C3)c(C(F)(F)F)cc1=O)c1cccc(c1)O2] |
| 7 | 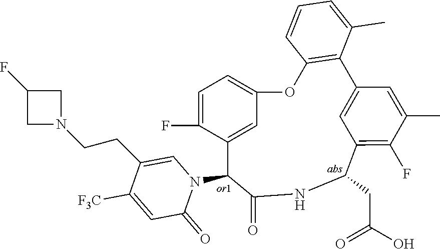 [SMILES: Cc1cc2cc(c1F)[C@H](CC(=O)O)NC(=O)[C@@H](n1cc(CCN3CC(F)C3)c(C(F)(F)F)cc1=O)c1cc(ccc1F)Oc1cccc(C)c1-2] |
| 8 | 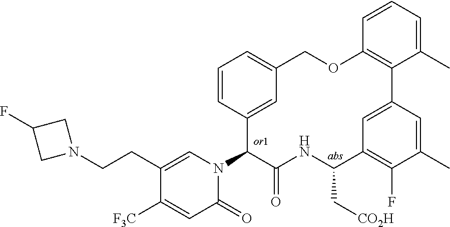 [SMILES: Cc1cc2cc(c1F)[C@H](CC(=O)O)NC(=O)[C@@H](n1cc(CCN3CC(F)C3)c(C(F)(F)F)cc1=O)c1cccc(c1)COc1cccc(C)c1-2] |
| 9 | 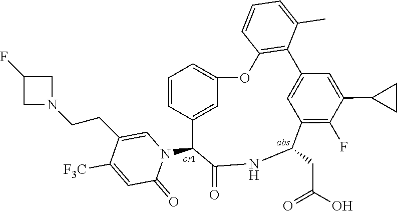 [SMILES: Cc1cccc2c1-c1cc(C3CC3)c(F)c(c1)[C@H](CC(=O)O)NC(=O)[C@@H](n1cc(CCN3CC(F)C3)c(C(F)(F)F)cc1=O)c1cccc(c1)O2] |
| 10 | 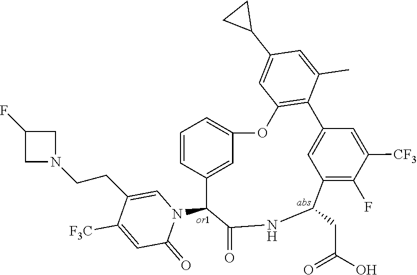 [SMILES: Cc1cc(C2CC2)cc2c1-c1cc(c(F)c(C(F)(F)F)c1)[C@H](CC(=O)O)NC(=O)[C@@H](n1cc(CCN3CC(F)C3)c(C(F)(F)F)cc1=O)c1cccc(c1)O2] |
| 11 | 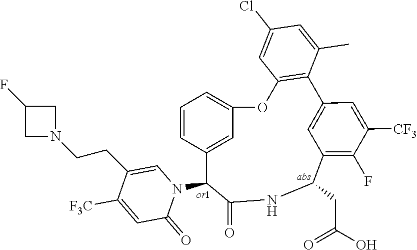 [SMILES: Cc1cc(Cl)cc2c1-c1cc(c(F)c(C(F)(F)F)c1)[C@H](CC(=O)O)NC(=O)[C@@H](n1cc(CCN3CC(F)C3)c(C(F)(F)F)cc1=O)c1cccc(c1)O2] |
| 12 | 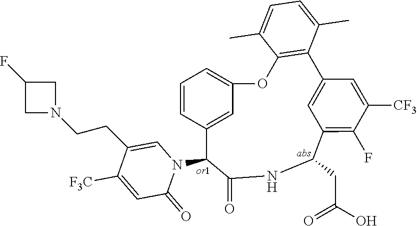 [SMILES: Cc1ccc(C)c2c1Oc1cccc(c1)[C@H](n1cc(CCN3CC(F)C3)c(C(F)(F)F)cc1=O)C(=O)N[C@@H](CC(=O)O)c1cc-2cc(C(F)(F)F)c1F] |
| 13 | 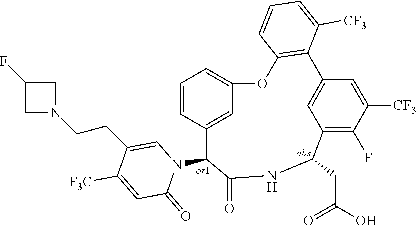 [SMILES: O=C(O)C[C@@H]1NC(=O)[C@@H](n2cc(CCN3CC(F)C3)c(C(F)(F)F)cc2=O)c2cccc(c2)Oc2cccc(C(F)(F)F)c2-c2cc1c(F)c(C(F)(F)F)c2] |
| 14 | 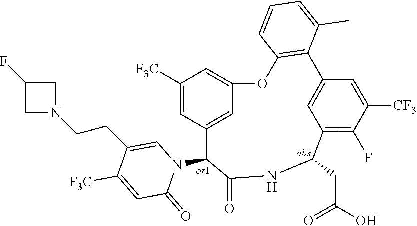 [SMILES: Cc1cccc2c1-c1cc(c(F)c(C(F)(F)F)c1)[C@H](CC(=O)O)NC(=O)[C@@H](n1cc(CCN3CC(F)C3)c(C(F)(F)F)cc1=O)c1cc(cc(C(F)(F)F)c1)O2] |
| 15 | 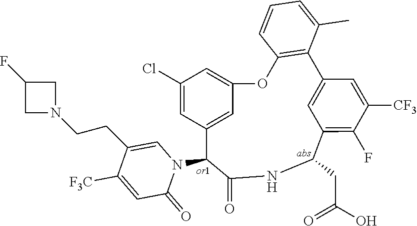 [SMILES: Cc1cccc2c1-c1cc(c(F)c(C(F)(F)F)c1)[C@H](CC(=O)O)NC(=O)[C@@H](n1cc(CCN3CC(F)C3)c(C(F)(F)F)cc1=O)c1cc(Cl)cc(c1)O2] |
| 16 | 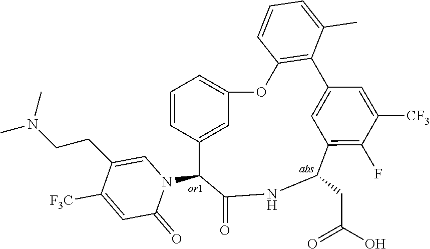 [SMILES: Cc1cccc2c1-c1cc(c(F)c(C(F)(F)F)c1)[C@H](CC(=O)O)NC(=O)[C@@H](n1cc(CCN(C)C)c(C(F)(F)F)cc1=O)c1cccc(c1)O2] |
| 17 | 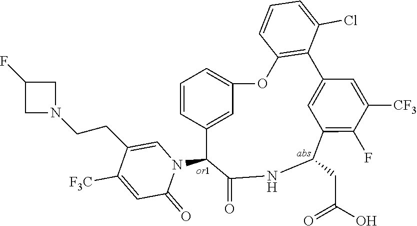 [SMILES: O=C(O)C[C@@H]1NC(=O)[C@@H](n2cc(CCN3CC(F)C3)c(C(F)(F)F)cc2=O)c2cccc(c2)Oc2cccc(Cl)c2-c2cc1c(F)c(C(F)(F)F)c2] |
| 18 | 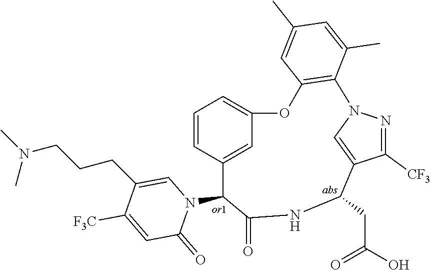 [SMILES: Cc1cc(C)c2c(c1)Oc1cccc(c1)[C@H](n1cc(CCCN(C)C)c(C(F)(F)F)cc1=O)C(=O)N[C@@H](CC(=O)O)c1cn-2nc1C(F)(F)F] |
| 19 | 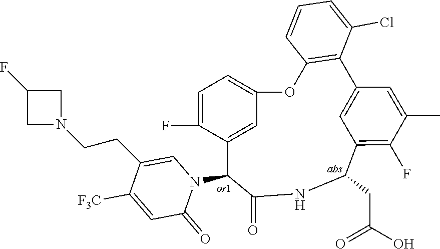 [SMILES: Cc1cc2cc(c1F)[C@H](CC(=O)O)NC(=O)[C@@H](n1cc(CCN3CC(F)C3)c(C(F)(F)F)cc1=O)c1cc(ccc1F)Oc1cccc(Cl)c1-2] |
| 20 | 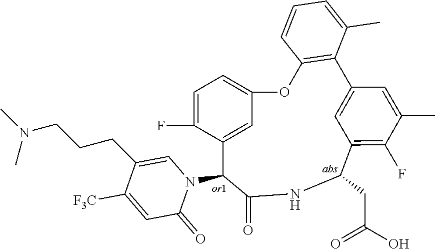 |
| 21 | 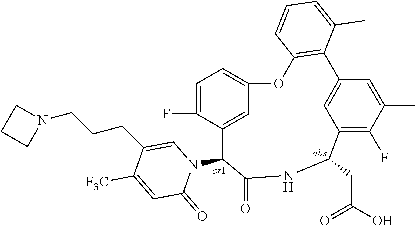 [SMILES: Cc1cc2cc(c1F)[C@H](CC(=O)O)NC(=O)[C@@H](n1cc(CCCN3CCC3)c(C(F)(F)F)cc1=O)c1cc(ccc1F)Oc1cccc(C)c1-2] |
| 22 | 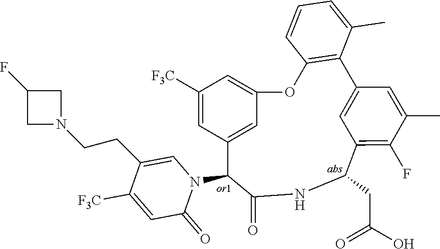 [SMILES: Cc1cc2cc(c1F)[C@H](CC(=O)O)NC(=O)[C@@H](n1cc(CCN3CC(F)C3)c(C(F)(F)F)cc1=O)c1cc(cc(C(F)(F)F)c1)Oc1cccc(C)c1-2] |
| 23 | 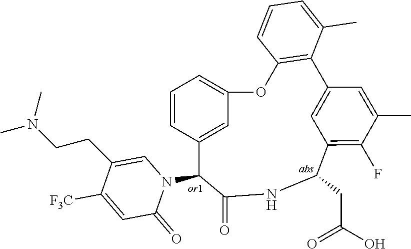 |
| 24 | 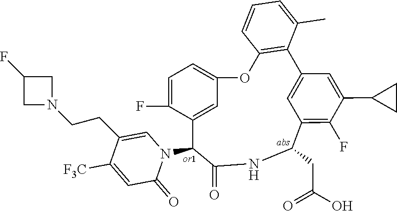 [SMILES: Cc1cccc2c1-c1cc(C3CC3)c(F)c(c1)[C@H](CC(=O)O)NC(=O)[C@@H](n1cc(CCN3CC(F)C3)c(C(F)(F)F)cc1=O)c1cc(ccc1F)O2] |
| 25 | 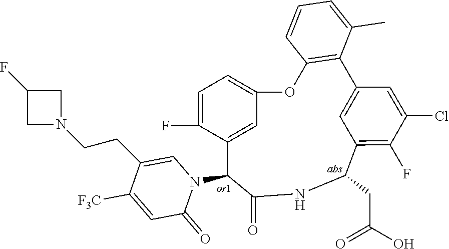 [SMILES: Cc1cccc2c1-c1cc(Cl)c(F)c(c1)[C@H](CC(=O)O)NC(=O)[C@@H](n1cc(CCN3CC(F)C3)c(C(F)(F)F)cc1=O)c1cc(ccc1F)O2] |
| 26 | 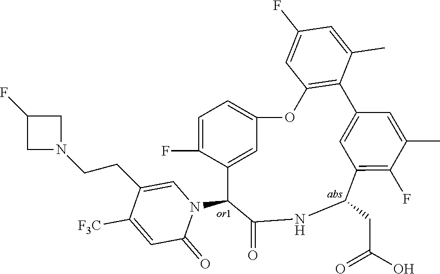 [SMILES: Cc1cc2cc(c1F)[C@H](CC(=O)O)NC(=O)[C@@H](n1cc(CCN3CC(F)C3)c(C(F)(F)F)cc1=O)c1cc(ccc1F)Oc1cc(F)cc(C)c1-2] |
| 27 | 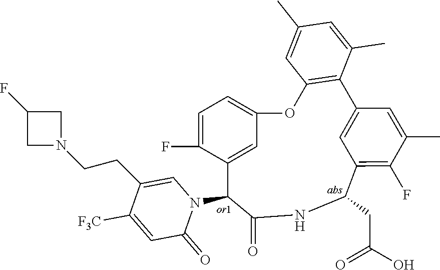 [SMILES: Cc1cc(C)c2c(c1)Oc1ccc(F)c(c1)[C@H](n1cc(CCN3CC(F)C3)c(C(F)(F)F)cc1=O)C(=O)N[C@@H](CC(=O)O)c1cc-2cc(C)c1F] |
| 28 | 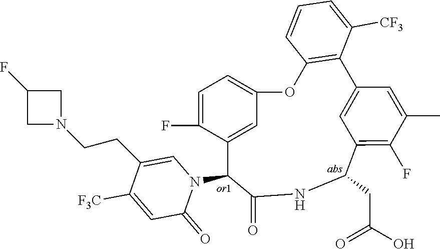 [SMILES: Cc1cc2cc(c1F)[C@H](CC(=O)O)NC(=O)[C@@H](n1cc(CCN3CC(F)C3)c(C(F)(F)F)cc1=O)c1cc(ccc1F)Oc1cccc(C(F)(F)F)c1-2] |
| 29 | 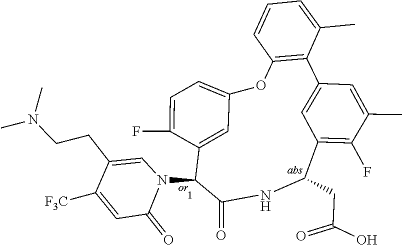 |
| 30 | 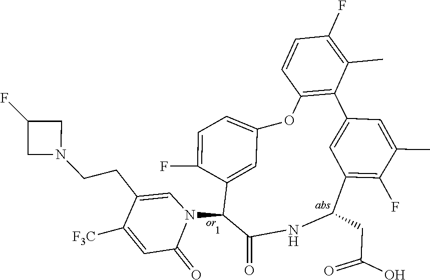 [SMILES: Cc1cc2cc(c1F)[C@H](CC(=O)O)NC(=O)[C@@H](n1cc(CCN3CC(F)C3)c(C(F)(F)F)cc1=O)c1cc(ccc1F)Oc1ccc(F)c(C)c1-2] |
| 31 | 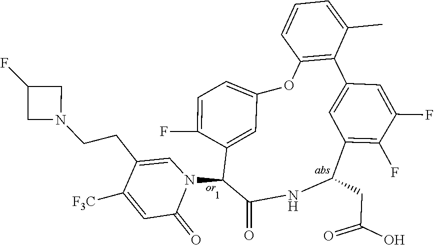 [SMILES: Cc1cccc2c1-c1cc(F)c(F)c(c1)[C@H](CC(=O)O)NC(=O)[C@@H](n1cc(CCN3CC(F)C3)c(C(F)(F)F)cc1=O)c1cc(ccc1F)O2] |
| 32 | 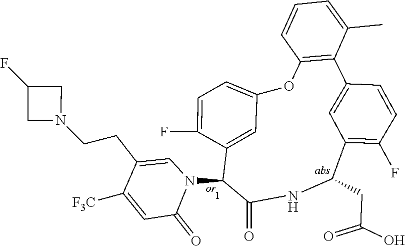 [SMILES: Cc1cccc2c1-c1ccc(F)c(c1)[C@H](CC(=O)O)NC(=O)[C@@H](n1cc(CCN3CC(F)C3)c(C(F)(F)F)cc1=O)c1cc(ccc1F)O2] |
| 33 | 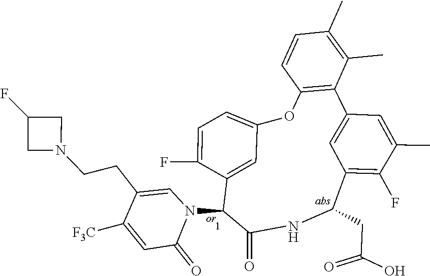 [SMILES: Cc1ccc2c(c1C)-c1cc(C)c(F)c(c1)[C@H](CC(=O)O)NC(=O)[C@@H](n1cc(CCN3CC(F)C3)c(C(F)(F)F)cc1=O)c1cc(ccc1F)O2] |
| 34 | 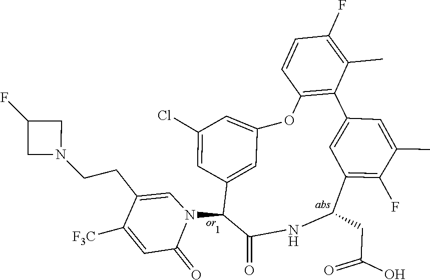 [SMILES: Cc1cc2cc(c1F)[C@H](CC(=O)O)NC(=O)[C@@H](n1cc(CCN3CC(F)C3)c(C(F)(F)F)cc1=O)c1cc(Cl)cc(c1)Oc1ccc(F)c(C)c1-2] |
| 35 | 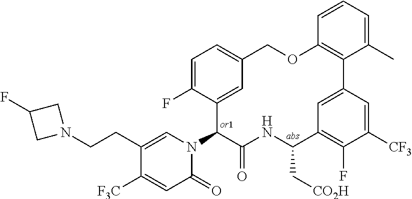 [SMILES: Cc1cccc2c1-c1cc(c(F)c(C(F)(F)F)c1)[C@H](CC(=O)O)NC(=O)[C@@H](n1cc(CCN3CC(F)C3)c(C(F)(F)F)cc1=O)c1cc(ccc1F)CO2] |
| 36 | 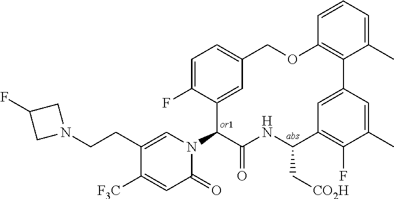 [SMILES: Cc1cc2cc(c1F)[C@H](CC(=O)O)NC(=O)[C@@H](n1cc(CCN3CC(F)C3)c(C(F)(F)F)cc1=O)c1cc(ccc1F)COc1cccc(C)c1-2] |
| 37 | 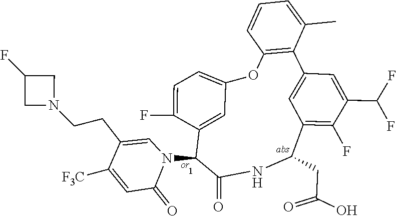 [SMILES: Cc1cccc2c1-c1cc(C(F)F)c(F)c(c1)[C@H](CC(=O)O)NC(=O)[C@@H](n1cc(CCN3CC(F)C3)c(C(F)(F)F)cc1=O)c1cc(ccc1F)O2] |
| 38 | 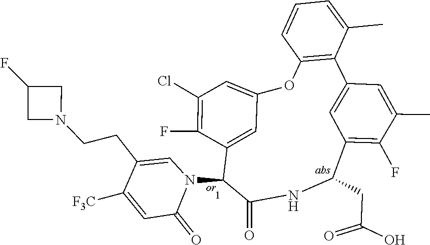 [SMILES: Cc1cc2cc(c1F)[C@H](CC(=O)O)NC(=O)[C@@H](n1cc(CCN3CC(F)C3)c(C(F)(F)F)cc1=O)c1cc(cc(Cl)c1F)Oc1cccc(C)c1-2] |
| 39 | 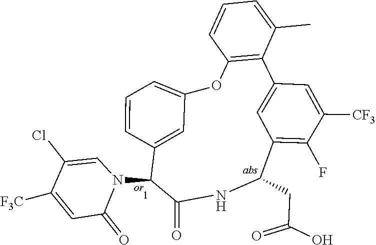 [SMILES: Cc1cccc2c1-c1cc(c(F)c(C(F)(F)F)c1)[C@H](CC(=O)O)NC(=O)[C@@H](n1cc(Cl)c(C(F)(F)F)cc1=O)c1cccc(c1)O2] |
| 40 | 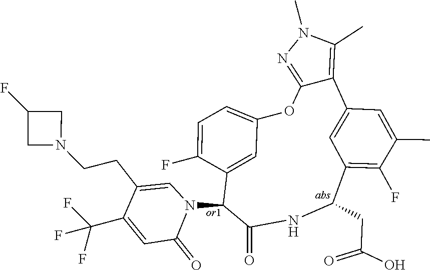 [SMILES: Cc1cc2cc(c1F)[C@H](CC(=O)O)NC(=O)[C@@H](n1cc(CCN3CC(F)C3)c(C(F)(F)F)cc1=O)c1cc(ccc1F)Oc1nn(C)c(C)c1-2] |
| 41 | 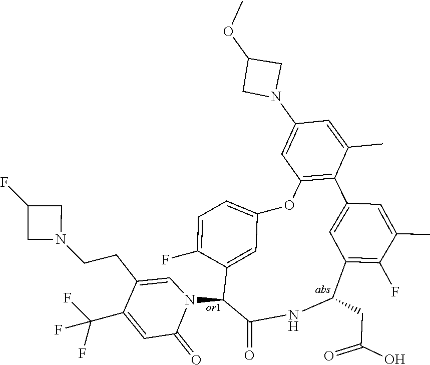 [SMILES: COC1CN(c2cc(C)c3c(c2)Oc2ccc(F)c(c2)[C@H](n2cc(CCN4CC(F)C4)c(C(F)(F)F)cc2=O)C(=O)N[C@@H](CC(=O)O)c2cc-3cc(C)c2F)C1] |
| 42 | 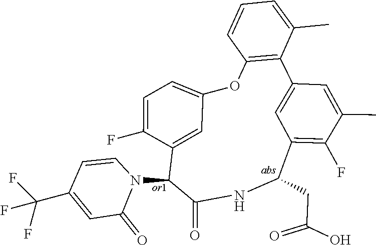 [SMILES: Cc1cc2cc(c1F)[C@H](CC(=O)O)NC(=O)[C@@H](n1ccc(C(F)(F)F)cc1=O)c1cc(ccc1F)Oc1cccc(C)c1-2] |
| 43 | 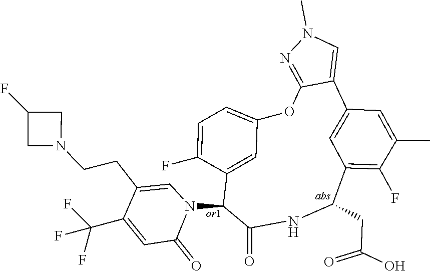 [SMILES: Cc1cc2cc(c1F)[C@H](CC(=O)O)NC(=O)[C@@H](n1cc(CCN3CC(F)C3)c(C(F)(F)F)cc1=O)c1cc(ccc1F)Oc1nn(C)cc1-2] |
| 44 | 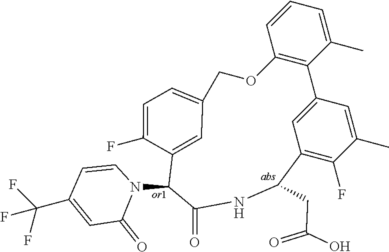 [SMILES: Cc1cc2cc(c1F)[C@H](CC(=O)O)NC(=O)[C@@H](n1ccc(C(F)(F)F)cc1=O)c1cc(ccc1F)COc1cccc(C)c1-2] |
| 45 | 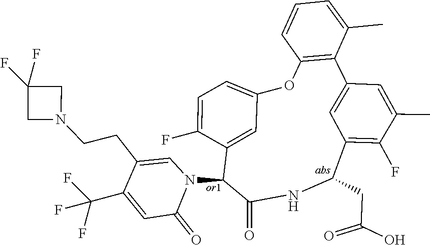 [SMILES: Cc1cc2cc(c1F)[C@H](CC(=O)O)NC(=O)[C@@H](n1cc(CCN3CC(F)(F)C3)c(C(F)(F)F)cc1=O)c1cc(ccc1F)Oc1cccc(C)c1-2] |
| 46 | 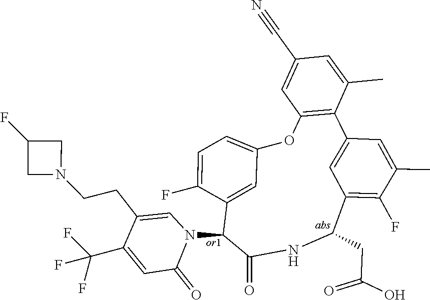 [SMILES: Cc1cc2cc(c1F)[C@H](CC(=O)O)NC(=O)[C@@H](n1cc(CCN3CC(F)C3)c(C(F)(F)F)cc1=O)c1cc(ccc1F)Oc1cc(C#N)cc(C)c1-2] |
| 47 | 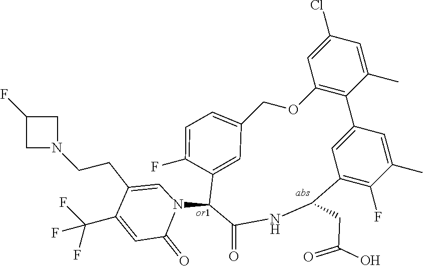 [SMILES: Cc1cc2cc(c1F)[C@H](CC(=O)O)NC(=O)[C@@H](n1cc(CCN3CC(F)C3)c(C(F)(F)F)cc1=O)c1cc(ccc1F)COc1cc(Cl)cc(C)c1-2] |
| 48 | 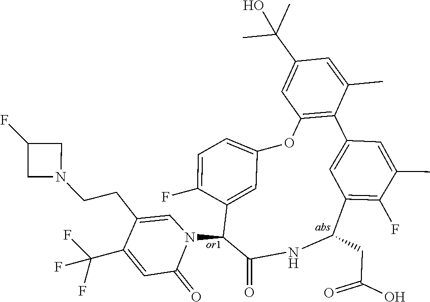 [SMILES: Cc1cc2cc(c1F)[C@H](CC(=O)O)NC(=O)[C@@H](n1cc(CCN3CC(F)C3)c(C(F)(F)F)cc1=O)c1cc(ccc1F)Oc1cc(C(C)(C)O)cc(C)c1-2] |
| 49 | 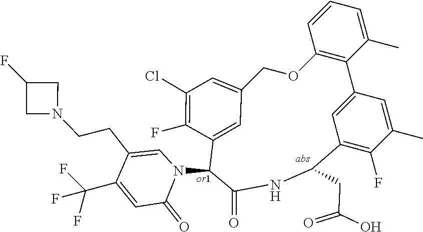 [SMILES: Cc1cc2cc(c1F)[C@H](CC(=O)O)NC(=O)[C@@H](n1cc(CCN3CC(F)C3)c(C(F)(F)F)cc1=O)c1cc(cc(Cl)c1F)COc1cccc(C)c1-2] |
| 50 | 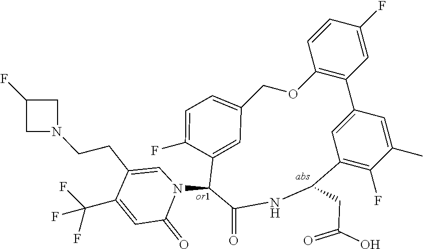 [SMILES: Cc1cc2cc(c1F)[C@H](CC(=O)O)NC(=O)[C@@H](n1cc(CCN3CC(F)C3)c(C(F)(F)F)cc1=O)c1cc(ccc1F)COc1ccc(F)cc1-2] |
| 51 | 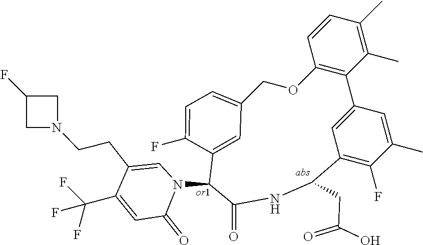 [SMILES: Cc1ccc2c(c1C)-c1cc(C)c(F)c(c1)[C@H](CC(=O)O)NC(=O)[C@@H](n1cc(CCN3CC(F)C3)c(C(F)(F)F)cc1=O)c1cc(ccc1F)CO2] |
| 52 | 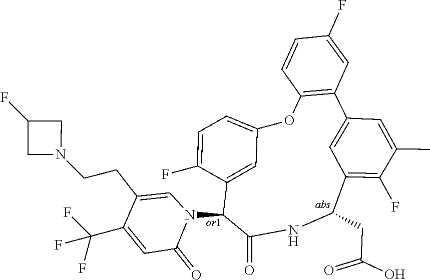 [SMILES: Cc1cc2cc(c1F)[C@H](CC(=O)O)NC(=O)[C@@H](n1cc(CCN3CC(F)C3)c(C(F)(F)F)cc1=O)c1cc(ccc1F)Oc1ccc(F)cc1-2] |
| 53 | 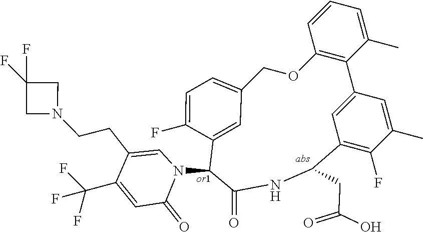 [SMILES: Cc1cc2cc(c1F)[C@H](CC(=O)O)NC(=O)[C@@H](n1cc(CCN3CC(F)(F)C3)c(C(F)(F)F)cc1=O)c1cc(ccc1F)COc1cccc(C)c1-2] |
| 54 | 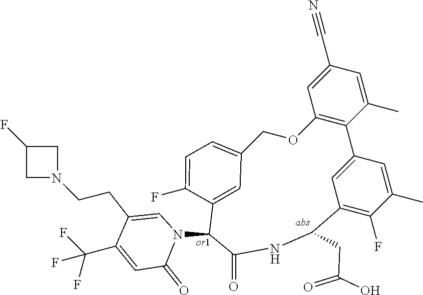 [SMILES: Cc1cc2cc(c1F)[C@H](CC(=O)O)NC(=O)[C@@H](n1cc(CCN3CC(F)C3)c(C(F)(F)F)cc1=O)c1cc(ccc1F)COc1cc(C#N)cc(C)c1-2] |
| 55 | 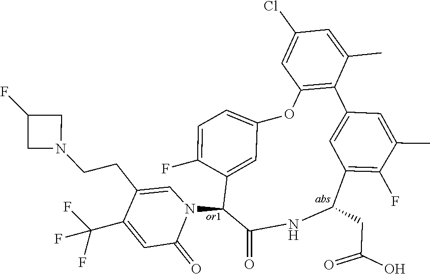 [SMILES: Cc1cc2cc(c1F)[C@H](CC(=O)O)NC(=O)[C@@H](n1cc(CCN3CC(F)C3)c(C(F)(F)F)cc1=O)c1cc(ccc1F)Oc1cc(Cl)cc(C)c1-2] |
| 56 | 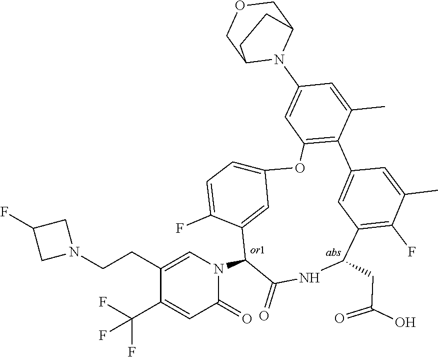 [SMILES: Cc1cc2cc(c1F)[C@H](CC(=O)O)NC(=O)[C@@H](n1cc(CCN3CC(F)C3)c(C(F)(F)F)cc1=O)c1cc(ccc1F)Oc1cc(N3C4CCC3COC4)cc(C)c1-2] |
| 57 | 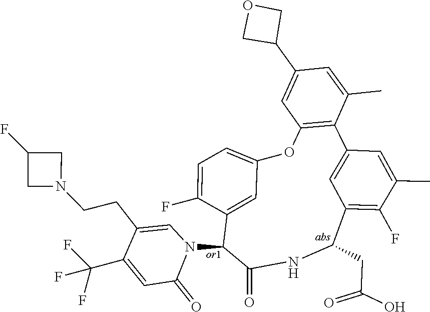 [SMILES: Cc1cc2cc(c1F)[C@H](CC(=O)O)NC(=O)[C@@H](n1cc(CCN3CC(F)C3)c(C(F)(F)F)cc1=O)c1cc(ccc1F)Oc1cc(C3COC3)cc(C)c1-2] |
| 58 | 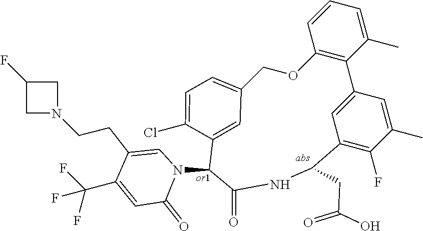 [SMILES: Cc1cc2cc(c1F)[C@H](CC(=O)O)NC(=O)[C@@H](n1cc(CCN3CC(F)C3)c(C(F)(F)F)cc1=O)c1cc(ccc1Cl)COc1cccc(C)c1-2] |
| 59 | 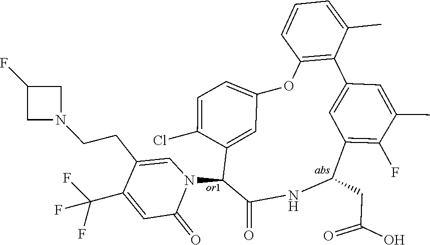 [SMILES: Cc1cc2cc(c1F)[C@H](CC(=O)O)NC(=O)[C@@H](n1cc(CCN3CC(F)C3)c(C(F)(F)F)cc1=O)c1cc(ccc1Cl)Oc1cccc(C)c1-2] |
| 60 | 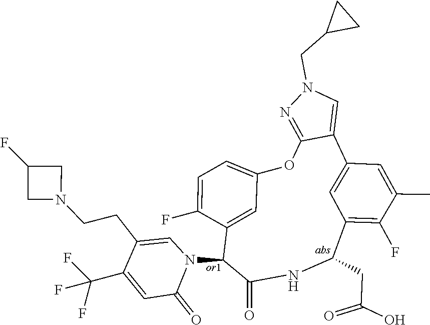 [SMILES: Cc1cc2cc(c1F)[C@H](CC(=O)O)NC(=O)[C@@H](n1cc(CCN3CC(F)C3)c(C(F)(F)F)cc1=O)c1cc(ccc1F)Oc1nn(CC3CC3)cc1-2] |
| 61 | 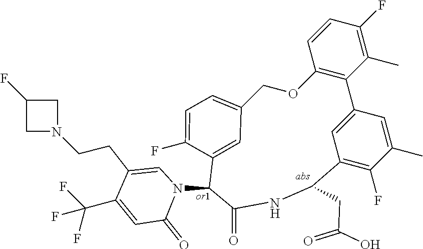 [SMILES: Cc1cc2cc(c1F)[C@H](CC(=O)O)NC(=O)[C@@H](n1cc(CCN3CC(F)C3)c(C(F)(F)F)cc1=O)c1cc(ccc1F)COc1ccc(F)c(C)c1-2] |
| 62 | 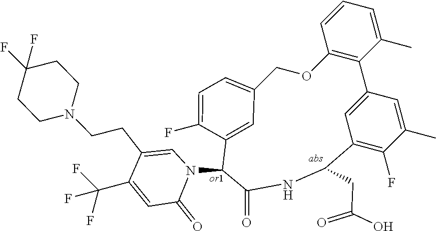 [SMILES: Cc1cc2cc(c1F)[C@H](CC(=O)O)NC(=O)[C@@H](n1cc(CCN3CCC(F)(F)CC3)c(C(F)(F)F)cc1=O)c1cc(ccc1F)COc1cccc(C)c1-2] |
| 63 | 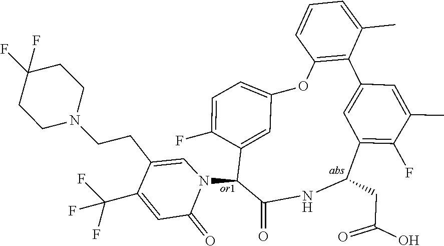 [SMILES: Cc1cc2cc(c1F)[C@H](CC(=O)O)NC(=O)[C@@H](n1cc(CCN3CCC(F)(F)CC3)c(C(F)(F)F)cc1=O)c1cc(ccc1F)Oc1cccc(C)c1-2] |
| 64 | 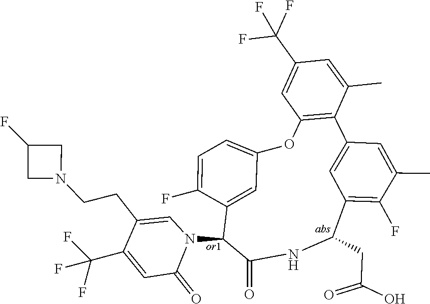 [SMILES: Cc1cc2cc(c1F)[C@H](CC(=O)O)NC(=O)[C@@H](n1cc(CCN3CC(F)C3)c(C(F)(F)F)cc1=O)c1cc(ccc1F)Oc1cc(C(F)(F)F)cc(C)c1-2] |
| 65 | 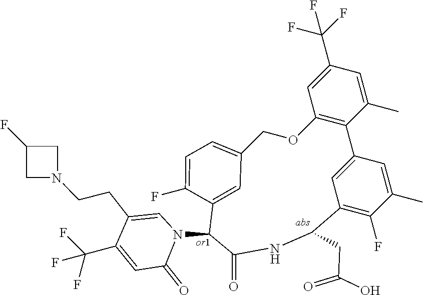 [SMILES: Cc1cc2cc(c1F)[C@H](CC(=O)O)NC(=O)[C@@H](n1cc(CCN3CC(F)C3)c(C(F)(F)F)cc1=O)c1cc(ccc1F)COc1cc(C(F)(F)F)cc(C)c1-2] |
| 66 | 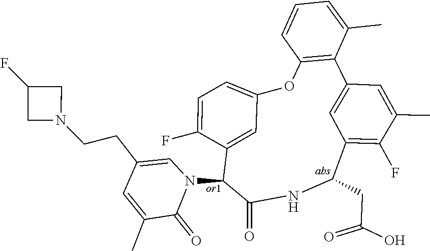 [SMILES: Cc1cc2cc(c1F)[C@H](CC(=O)O)NC(=O)[C@@H](n1cc(CCN3CC(F)C3)cc(C)c1=O)c1cc(ccc1F)Oc1cccc(C)c1-2] |
| 67 | 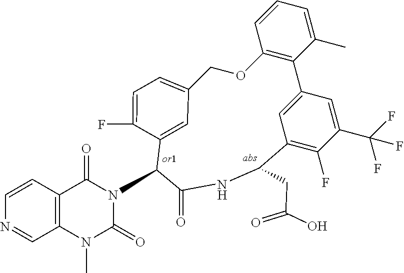 [SMILES: Cc1cccc2c1-c1cc(c(F)c(C(F)(F)F)c1)[C@H](CC(=O)O)NC(=O)[C@@H](n1c(=O)c3ccncc3n(C)c1=O)c1cc(ccc1F)CO2] |
| 68 | 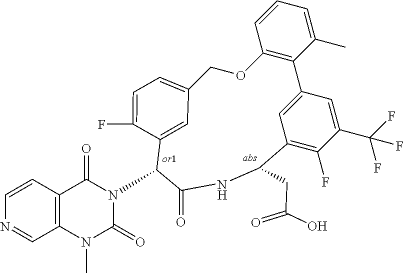 [SMILES: Cc1cccc2c1-c1cc(c(F)c(C(F)(F)F)c1)[C@H](CC(=O)O)NC(=O)[C@H](n1c(=O)c3ccncc3n(C)c1=O)c1cc(ccc1F)CO2] |
| 69 | 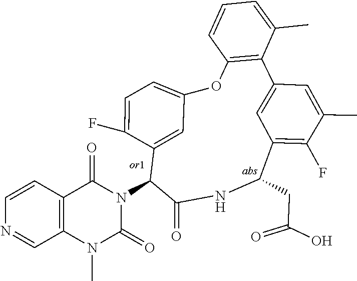 [SMILES: Cc1cc2cc(c1F)[C@H](CC(=O)O)NC(=O)[C@@H](n1c(=O)c3ccncc3n(C)c1=O)c1cc(ccc1F)Oc1cccc(C)c1-2] |
| 70 | 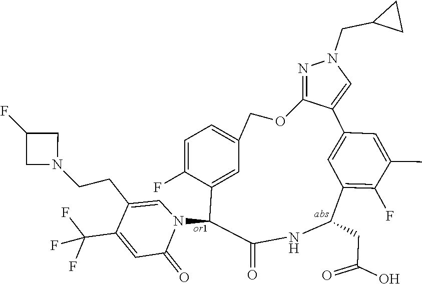 [SMILES: Cc1cc2cc(c1F)[C@H](CC(=O)O)NC(=O)[C@@H](n1cc(CCN3CC(F)C3)c(C(F)(F)F)cc1=O)c1cc(ccc1F)COc1nn(CC3CC3)cc1-2] |
| 71 | 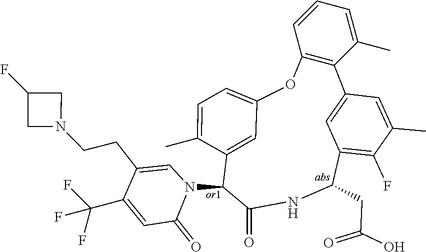 [SMILES: Cc1ccc2cc1[C@H](n1cc(CCN3CC(F)C3)c(C(F)(F)F)cc1=O)C(=O)N[C@@H](CC(=O)O)c1cc(cc(C)c1F)-c1c(C)cccc1O2] |
| 72 | 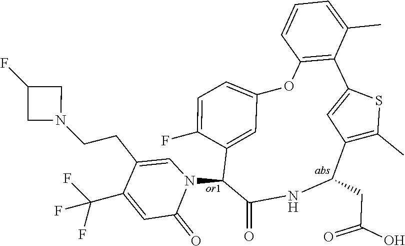 [SMILES: Cc1cccc2c1-c1cc(c(C)s1)[C@H](CC(=O)O)NC(=O)[C@@H](n1cc(CCN3CC(F)C3)c(C(F)(F)F)cc1=O)c1cc(ccc1F)O2] |
| 73 | 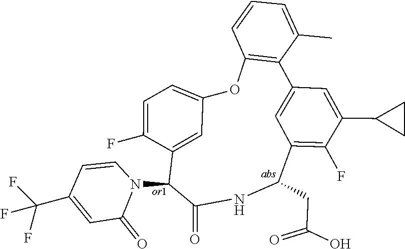 [SMILES: Cc1cccc2c1-c1cc(C3CC3)c(F)c(c1)[C@H](CC(=O)O)NC(=O)[C@@H](n1ccc(C(F)(F)F)cc1=O)c1cc(ccc1F)O2] |
| 74 | 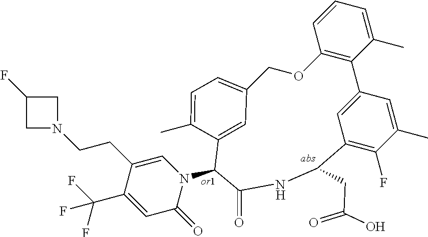 [SMILES: Cc1ccc2cc1[C@H](n1cc(CCN3CC(F)C3)c(C(F)(F)F)cc1=O)C(=O)N[C@@H](CC(=O)O)c1cc(cc(C)c1F)-c1c(C)cccc1OC2] |
| 75 | 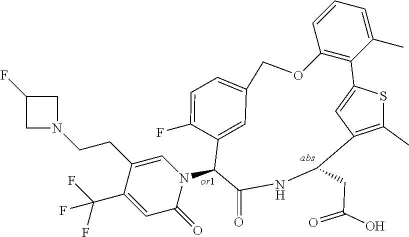 [SMILES: Cc1cccc2c1-c1cc(c(C)s1)[C@H](CC(=O)O)NC(=O)[C@@H](n1cc(CCN3CC(F)C3)c(C(F)(F)F)cc1=O)c1cc(ccc1F)CO2] |
| 76 | 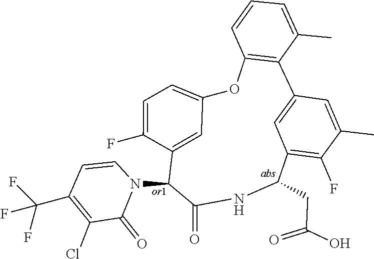 [SMILES: Cc1cc2cc(c1F)[C@H](CC(=O)O)NC(=O)[C@@H](n1ccc(C(F)(F)F)c(Cl)c1=O)c1cc(ccc1F)Oc1cccc(C)c1-2] |
| 77 | 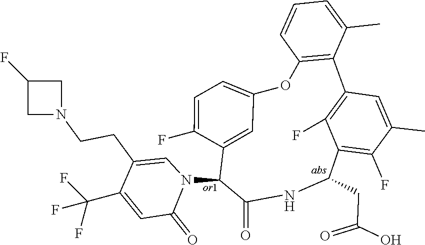 [SMILES: Cc1cc2c(F)c(c1F)[C@H](CC(=O)O)NC(=O)[C@@H](n1cc(CCN3CC(F)C3)c(C(F)(F)F)cc1=O)c1cc(ccc1F)Oc1cccc(C)c1-2] |
| 78 | 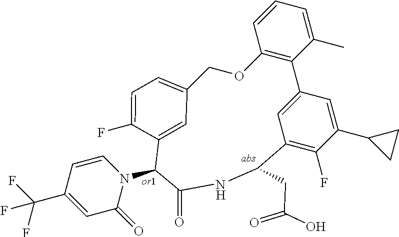 [SMILES: Cc1cccc2c1-c1cc(C3CC3)c(F)c(c1)[C@H](CC(=O)O)NC(=O)[C@@H](n1ccc(C(F)(F)F)cc1=O)c1cc(ccc1F)CO2] |
| 79 | 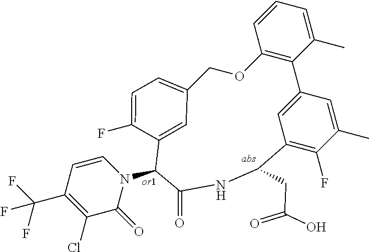 [SMILES: Cc1cc2cc(c1F)[C@H](CC(=O)O)NC(=O)[C@@H](n1ccc(C(F)(F)F)c(Cl)c1=O)c1cc(ccc1F)COc1cccc(C)c1-2] |
| 80 | 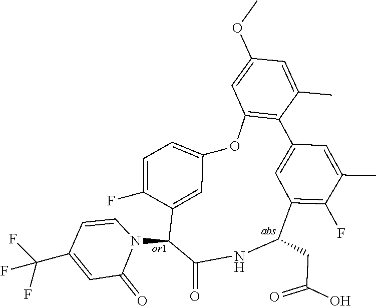 [SMILES: COc1cc(C)c2c(c1)Oc1ccc(F)c(c1)[C@H](n1ccc(C(F)(F)F)cc1=O)C(=O)N[C@@H](CC(=O)O)c1cc-2cc(C)c1F] |
| 81 | 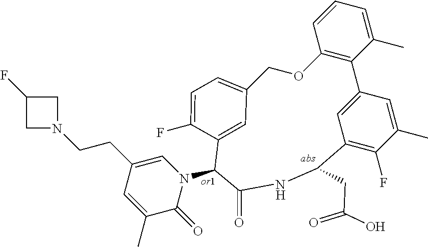 [SMILES: Cc1cc2cc(c1F)[C@H](CC(=O)O)NC(=O)[C@@H](n1cc(CCN3CC(F)C3)cc(C)c1=O)c1cc(ccc1F)COc1cccc(C)c1-2] |
| 82 | 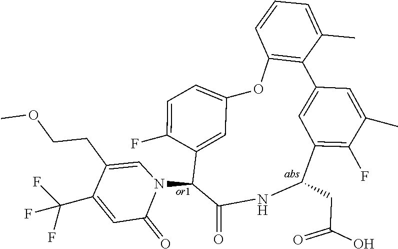 |
| 83 | 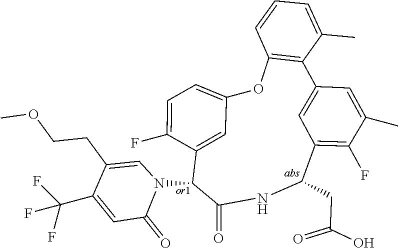 |
| 84 | 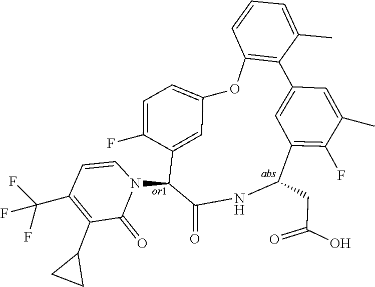 [SMILES: Cc1cc2cc(c1F)[C@H](CC(=O)O)NC(=O)[C@@H](n1ccc(C(F)(F)F)c(C3CC3)c1=O)c1cc(ccc1F)Oc1cccc(C)c1-2] |
| 85 | 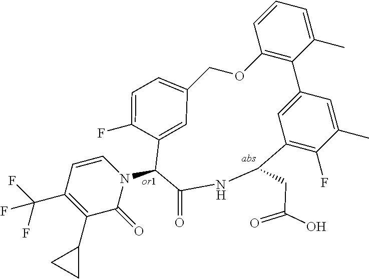 [SMILES: Cc1cc2cc(c1F)[C@H](CC(=O)O)NC(=O)[C@@H](n1ccc(C(F)(F)F)c(C3CC3)c1=O)c1cc(ccc1F)COc1cccc(C)c1-2] |
| 86 | 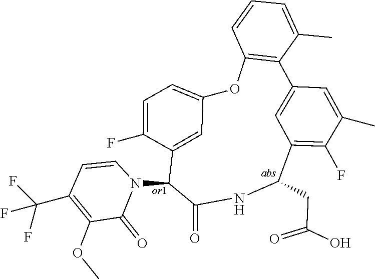 [SMILES: COc1c(C(F)(F)F)ccn([C@@H]2C(=O)N[C@@H](CC(=O)O)c3cc(cc(C)c3F)-c3c(C)cccc3Oc3ccc(F)c2c3)c1=O] |
| 87 | 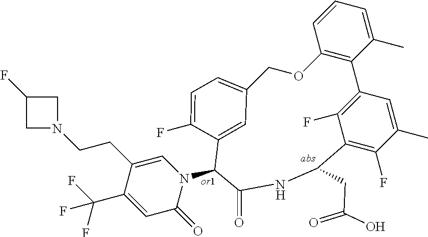 [SMILES: Cc1cc2c(F)c(c1F)[C@H](CC(=O)O)NC(=O)[C@@H](n1cc(CCN3CC(F)C3)c(C(F)(F)F)cc1=O)c1cc(ccc1F)COc1cccc(C)c1-2] |
| 88 | 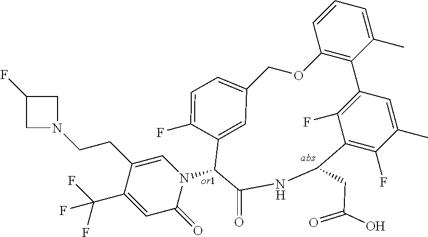 [SMILES: Cc1cc2c(F)c(c1F)[C@H](CC(=O)O)NC(=O)[C@H](n1cc(CCN3CC(F)C3)c(C(F)(F)F)cc1=O)c1cc(ccc1F)COc1cccc(C)c1-2] |
| 89 | 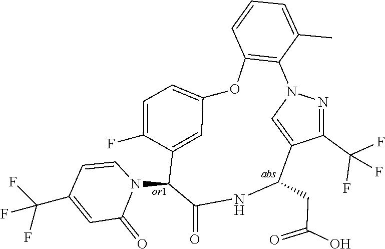 [SMILES: Cc1cccc2c1-n1cc(c(C(F)(F)F)n1)[C@H](CC(=O)O)NC(=O)[C@@H](n1ccc(C(F)(F)F)cc1=O)c1cc(ccc1F)O2] |
| 90 | 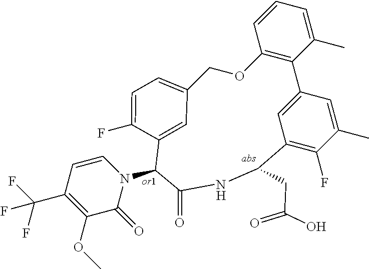 [SMILES: COc1c(C(F)(F)F)ccn([C@@H]2C(=O)N[C@@H](CC(=O)O)c3cc(cc(C)c3F)-c3c(C)cccc3OCc3ccc(F)c2c3)c1=O] |
| 91 | 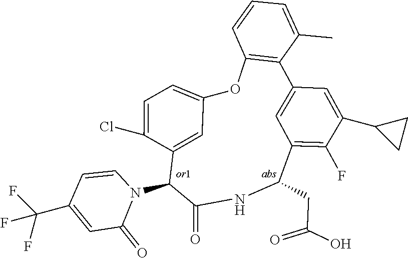 [SMILES: Cc1cccc2c1-c1cc(C3CC3)c(F)c(c1)[C@H](CC(=O)O)NC(=O)[C@@H](n1ccc(C(F)(F)F)cc1=O)c1cc(ccc1Cl)O2] |
| 92 | 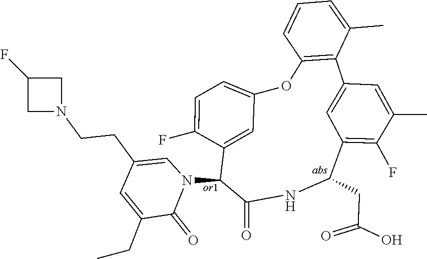 [SMILES: CCc1cc(CCN2CC(F)C2)cn([C@@H]2C(=O)N[C@@H](CC(=O)O)c3cc(cc(C)c3F)-c3c(C)cccc3Oc3ccc(F)c2c3)c1=O] |
| 93 | 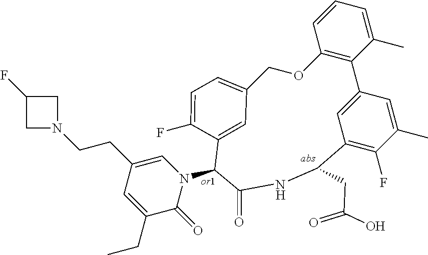 [SMILES: CCc1cc(CCN2CC(F)C2)cn([C@@H]2C(=O)N[C@@H](CC(=O)O)c3cc(cc(C)c3F)-c3c(C)cccc3OCc3ccc(F)c2c3)c1=O] |
| 94 | 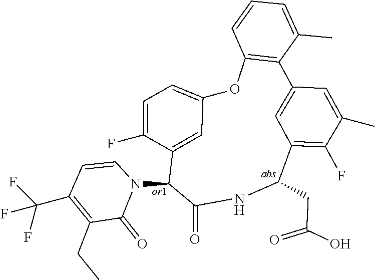 [SMILES: CCc1c(C(F)(F)F)ccn([C@@H]2C(=O)N[C@@H](CC(=O)O)c3cc(cc(C)c3F)-c3c(C)cccc3Oc3ccc(F)c2c3)c1=O] |
| 95 | 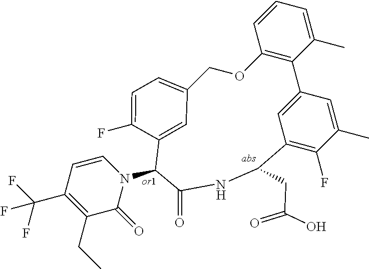 [SMILES: CCc1c(C(F)(F)F)ccn([C@@H]2C(=O)N[C@@H](CC(=O)O)c3cc(cc(C)c3F)-c3c(C)cccc3OCc3ccc(F)c2c3)c1=O] |
| 96 | 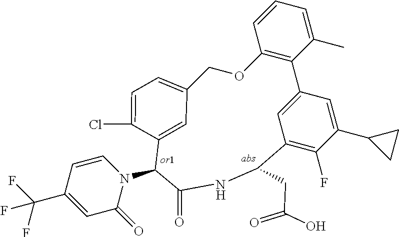 [SMILES: Cc1cccc2c1-c1cc(C3CC3)c(F)c(c1)[C@H](CC(=O)O)NC(=O)[C@@H](n1ccc(C(F)(F)F)cc1=O)c1cc(ccc1Cl)CO2] |
| 97 | 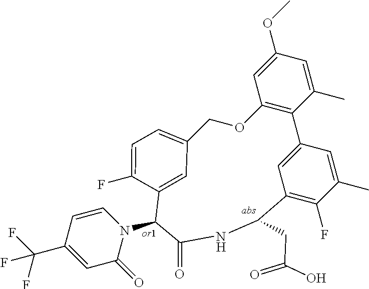 [SMILES: COc1cc(C)c2c(c1)OCc1ccc(F)c(c1)[C@H](n1ccc(C(F)(F)F)cc1=O)C(=O)N[C@@H](CC(=O)O)c1cc-2cc(C)c1F] |
| 98 | 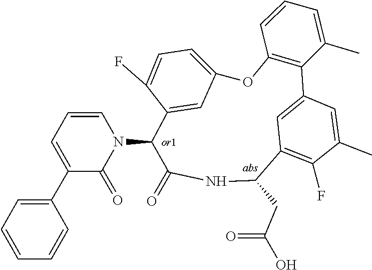 [SMILES: Cc1cc2cc(c1F)[C@H](CC(=O)O)NC(=O)[C@@H](n1cccc(-c3ccccc3)c1=O)c1cc(ccc1F)Oc1cccc(C)c1-2] |
| 99 | 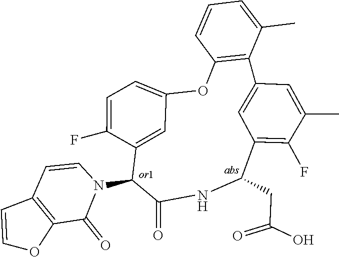 [SMILES: Cc1cc2cc(c1F)[C@H](CC(=O)O)NC(=O)[C@@H](n1ccc3ccoc3c1=O)c1cc(ccc1F)Oc1cccc(C)c1-2] |
| 100 | 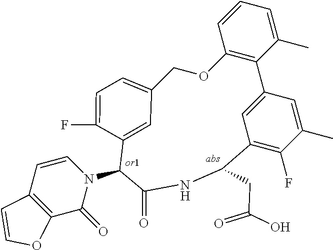 [SMILES: Cc1cc2cc(c1F)[C@H](CC(=O)O)NC(=O)[C@@H](n1ccc3ccoc3c1=O)c1cc(ccc1F)COc1cccc(C)c1-2] |
| 101 | 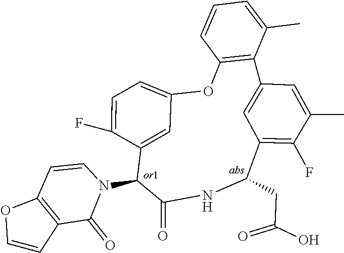 [SMILES: Cc1cc2cc(c1F)[C@H](CC(=O)O)NC(=O)[C@@H](n1ccc3occc3c1=O)c1cc(ccc1F)Oc1cccc(C)c1-2] |
| 102 | 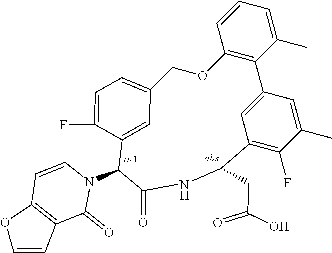 [SMILES: Cc1cc2cc(c1F)[C@H](CC(=O)O)NC(=O)[C@@H](n1ccc3occc3c1=O)c1cc(ccc1F)COc1cccc(C)c1-2] |
| 103 | 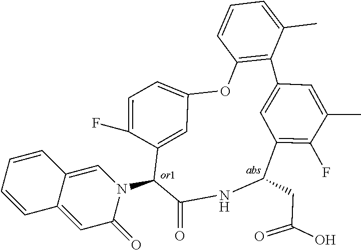 [SMILES: Cc1cc2cc(c1F)[C@H](CC(=O)O)NC(=O)[C@@H](n1cc3ccccc3cc1=O)c1cc(ccc1F)Oc1cccc(C)c1-2] |

The absolute label (abs) is added to a chiral center to denote that it is unambiguously a pure sample of the drawn stereoisomer.

The OR label (or) denotes a pure substance, but the absolute configuration of the stereochemical center is unknown. After chiral separation with pure structures isolated, multiple OR labels (OR indicates purity) with the same numerical value will indicates that a sample is one of a pair of pure enantiomers (but the absolute configuration of the stereochemical center is unknown).

The AND label (and) denotes both isomers are present at the depicted stereochemical center. Assigning different numerical values to the AND labels denotes that they are independent of each other. The use of AND labels with the same values indicate that the two stereocenters are relative to each other and can only change in concert.

In some embodiments, the compound disclosed herein, or a pharmaceutically acceptable salt, solvate, or stereoisomer thereof, is selected from a compound found in Table 2.

TABLE 2

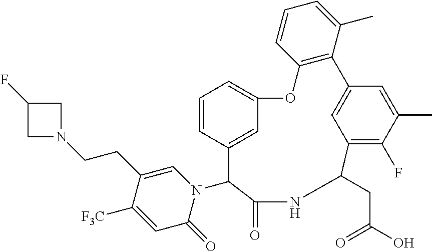 [SMILES: Cc1cc2cc(c1F)C(CC(=O)O)NC(=O)C(n1cc(CCN3CC(F)C3)c(C(F)(F)F)cc1=O)c1cccc(c1)Oc1cccc(C)c1-2]

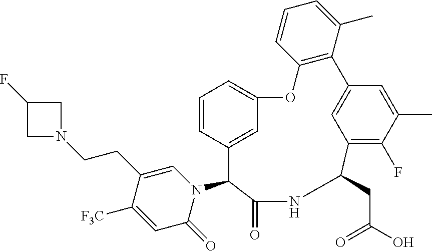 [SMILES: Cc1cc2cc(c1F)[C@@H](CC(=O)O)NC(=O)[C@@H](n1cc(CCN3CC(F)C3)c(C(F)(F)F)cc1=O)c1cccc(c1)Oc1cccc(C)c1-2]

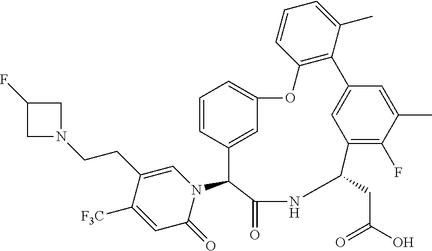 [SMILES: Cc1cc2cc(c1F)[C@H](CC(=O)O)NC(=O)[C@@H](n1cc(CCN3CC(F)C3)c(C(F)(F)F)cc1=O)c1cccc(c1)Oc1cccc(C)c1-2]

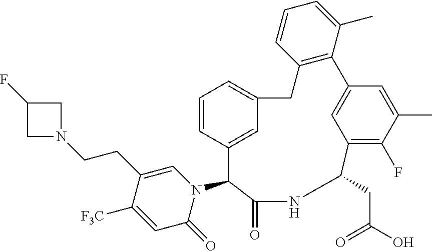 [SMILES: Cc1cc2cc(c1F)[C@H](CC(=O)O)NC(=O)[C@@H](n1cc(CCN3CC(F)C3)c(C(F)(F)F)cc1=O)c1cccc(c1)Cc1cccc(C)c1-2]

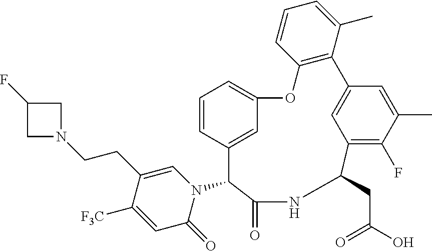 [SMILES: Cc1cc2cc(c1F)[C@@H](CC(=O)O)NC(=O)[C@H](n1cc(CCN3CC(F)C3)c(C(F)(F)F)cc1=O)c1cccc(c1)Oc1cccc(C)c1-2]

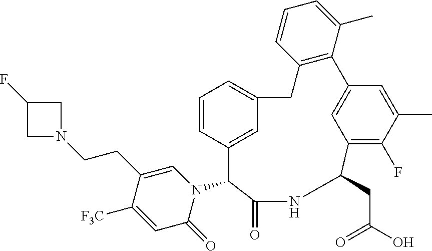 [SMILES: Cc1cc2cc(c1F)[C@@H](CC(=O)O)NC(=O)[C@H](n1cc(CCN3CC(F)C3)c(C(F)(F)F)cc1=O)c1cccc(c1)Cc1cccc(C)c1-2]

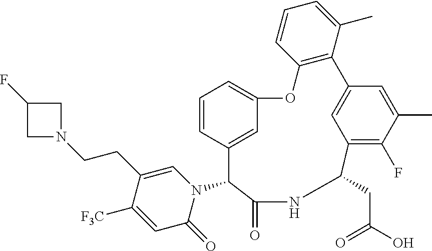 [SMILES: Cc1cc2cc(c1F)[C@H](CC(=O)O)NC(=O)[C@H](n1cc(CCN3CC(F)C3)c(C(F)(F)F)cc1=O)c1cccc(c1)Oc1cccc(C)c1-2]

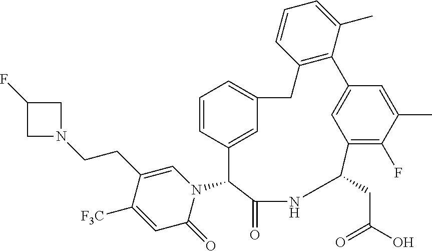 [SMILES: Cc1cc2cc(c1F)[C@H](CC(=O)O)NC(=O)[C@H](n1cc(CCN3CC(F)C3)c(C(F)(F)F)cc1=O)c1cccc(c1)Cc1cccc(C)c1-2]

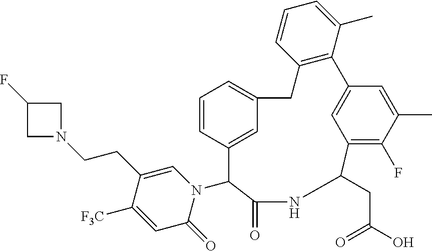 [SMILES: Cc1cc2cc(c1F)C(CC(=O)O)NC(=O)C(n1cc(CCN3CC(F)C3)c(C(F)(F)F)cc1=O)c1cccc(c1)Cc1cccc(C)c1-2]

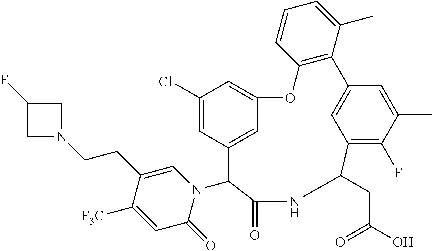 [SMILES: Cc1cc2cc(c1F)C(CC(=O)O)NC(=O)C(n1cc(CCN3CC(F)C3)c(C(F)(F)F)cc1=O)c1cc(Cl)cc(c1)Oc1cccc(C)c1-2]

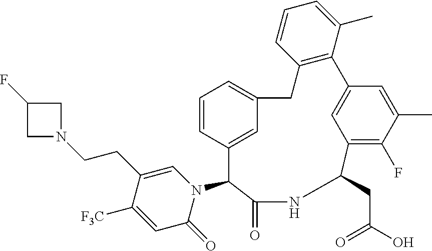 [SMILES: Cc1cc2cc(c1F)[C@@H](CC(=O)O)NC(=O)[C@@H](n1cc(CCN3CC(F)C3)c(C(F)(F)F)cc1=O)c1cccc(c1)Cc1cccc(C)c1-2]

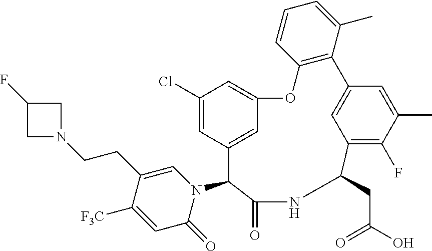 [SMILES: Cc1cc2cc(c1F)[C@@H](CC(=O)O)NC(=O)[C@@H](n1cc(CCN3CC(F)C3)c(C(F)(F)F)cc1=O)c1cc(Cl)cc(c1)Oc1cccc(C)c1-2]

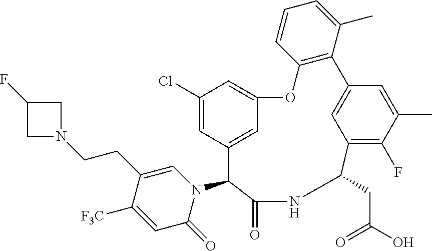 [SMILES: Cc1cc2cc(c1F)[C@H](CC(=O)O)NC(=O)[C@@H](n1cc(CCN3CC(F)C3)c(C(F)(F)F)cc1=O)c1cc(Cl)cc(c1)Oc1cccc(C)c1-2]

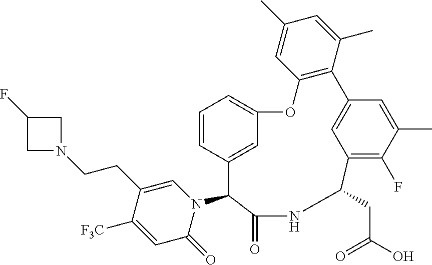 [SMILES: Cc1cc(C)c2c(c1)Oc1cccc(c1)[C@H](n1cc(CCN3CC(F)C3)c(C(F)(F)F)cc1=O)C(=O)N[C@@H](CC(=O)O)c1cc-2cc(C)c1F]

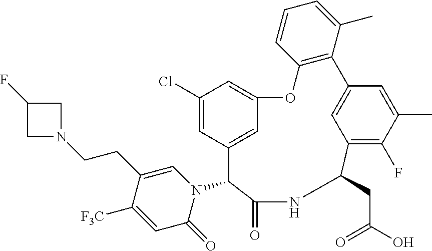 [SMILES: Cc1cc2cc(c1F)[C@@H](CC(=O)O)NC(=O)[C@H](n1cc(CCN3CC(F)C3)c(C(F)(F)F)cc1=O)c1cc(Cl)cc(c1)Oc1cccc(C)c1-2]

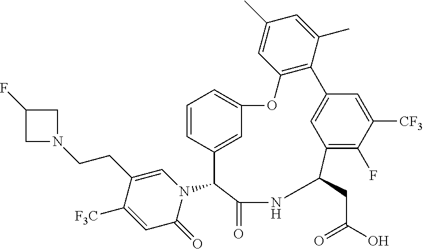 [SMILES: Cc1cc(C)c2c(c1)Oc1cccc(c1)[C@@H](n1cc(CCN3CC(F)C3)c(C(F)(F)F)cc1=O)C(=O)N[C@H](CC(=O)O)c1cc-2cc(C(F)(F)F)c1F]

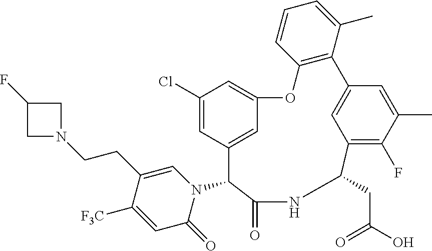 [SMILES: Cc1cc2cc(c1F)[C@H](CC(=O)O)NC(=O)[C@H](n1cc(CCN3CC(F)C3)c(C(F)(F)F)cc1=O)c1cc(Cl)cc(c1)Oc1cccc(C)c1-2]

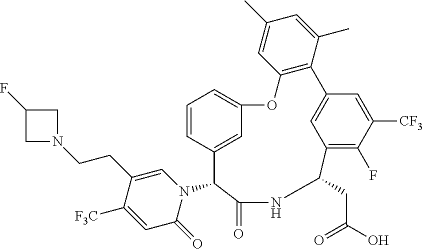 [SMILES: Cc1cc(C)c2c(c1)Oc1cccc(c1)[C@@H](n1cc(CCN3CC(F)C3)c(C(F)(F)F)cc1=O)C(=O)N[C@@H](CC(=O)O)c1cc-2cc(C(F)(F)F)c1F]

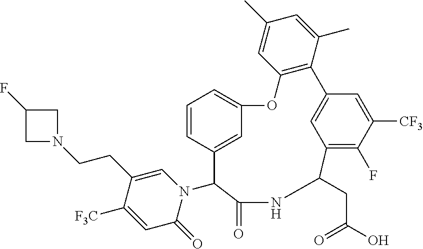 [SMILES: Cc1cc(C)c2c(c1)Oc1cccc(c1)C(n1cc(CCN3CC(F)C3)c(C(F)(F)F)cc1=O)C(=O)NC(CC(=O)O)c1cc-2cc(C(F)(F)F)c1F]

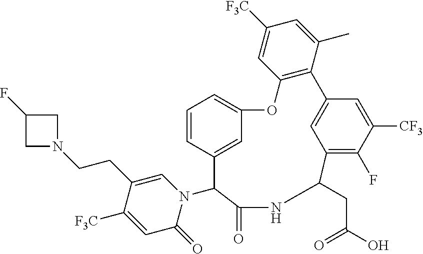 [SMILES: Cc1cc(C(F)(F)F)cc2c1-c1cc(c(F)c(C(F)(F)F)c1)C(CC(=O)O)NC(=O)C(n1cc(CCN3CC(F)C3)c(C(F)(F)F)cc1=O)c1cccc(c1)O2]

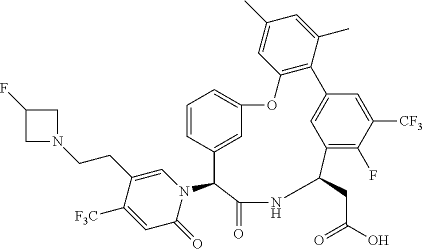 [SMILES: Cc1cc(C)c2c(c1)Oc1cccc(c1)[C@H](n1cc(CCN3CC(F)C3)c(C(F)(F)F)cc1=O)C(=O)N[C@H](CC(=O)O)c1cc-2cc(C(F)(F)F)c1F]

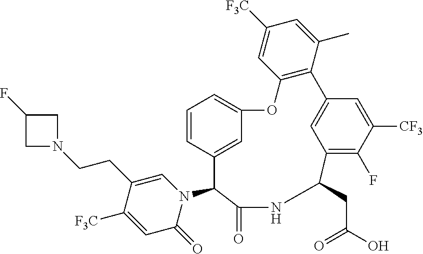 [SMILES: Cc1cc(C(F)(F)F)cc2c1-c1cc(c(F)c(C(F)(F)F)c1)[C@@H](CC(=O)O)NC(=O)[C@@H](n1cc(CCN3CC(F)C3)c(C(F)(F)F)cc1=O)c1cccc(c1)O2]

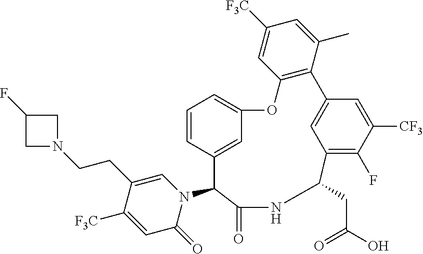 [SMILES: Cc1cc(C(F)(F)F)cc2c1-c1cc(c(F)c(C(F)(F)F)c1)[C@H](CC(=O)O)NC(=O)[C@@H](n1cc(CCN3CC(F)C3)c(C(F)(F)F)cc1=O)c1cccc(c1)O2]

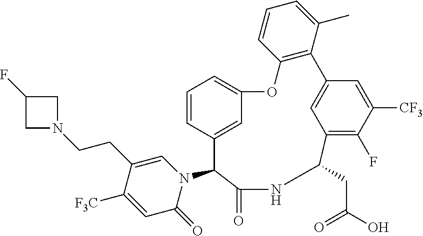 [SMILES: Cc1cccc2c1-c1cc(c(F)c(C(F)(F)F)c1)[C@H](CC(=O)O)NC(=O)[C@@H](n1cc(CCN3CC(F)C3)c(C(F)(F)F)cc1=O)c1cccc(c1)O2]

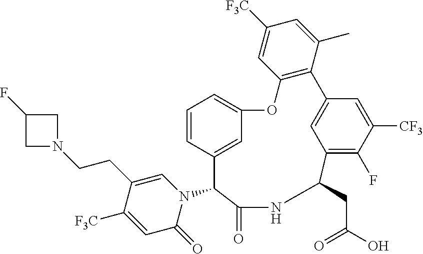 [SMILES: Cc1cc(C(F)(F)F)cc2c1-c1cc(c(F)c(C(F)(F)F)c1)[C@@H](CC(=O)O)NC(=O)[C@H](n1cc(CCN3CC(F)C3)c(C(F)(F)F)cc1=O)c1cccc(c1)O2]

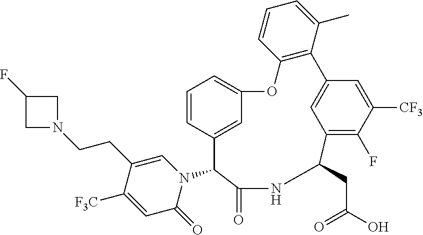 [SMILES: Cc1cccc2c1-c1cc(c(F)c(C(F)(F)F)c1)[C@@H](CC(=O)O)NC(=O)[C@H](n1cc(CCN3CC(F)C3)c(C(F)(F)F)cc1=O)c1cccc(c1)O2]

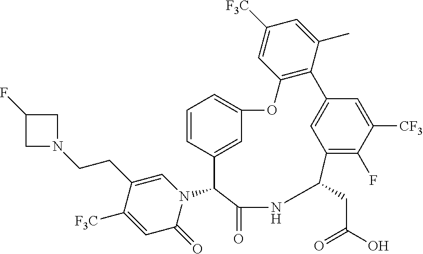 [SMILES: Cc1cc(C(F)(F)F)cc2c1-c1cc(c(F)c(C(F)(F)F)c1)[C@H](CC(=O)O)NC(=O)[C@H](n1cc(CCN3CC(F)C3)c(C(F)(F)F)cc1=O)c1cccc(c1)O2]

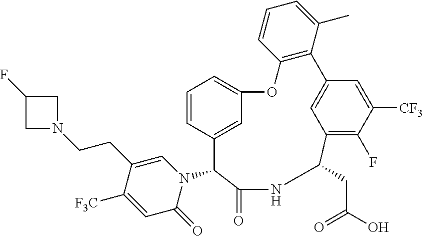 [SMILES: Cc1cccc2c1-c1cc(c(F)c(C(F)(F)F)c1)[C@H](CC(=O)O)NC(=O)[C@H](n1cc(CCN3CC(F)C3)c(C(F)(F)F)cc1=O)c1cccc(c1)O2]

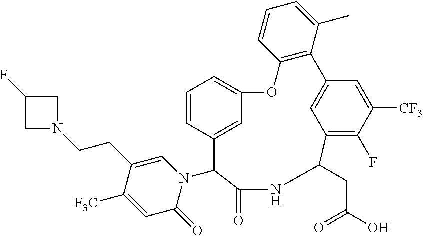 [SMILES: Cc1cccc2c1-c1cc(c(F)c(C(F)(F)F)c1)C(CC(=O)O)NC(=O)C(n1cc(CCN3CC(F)C3)c(C(F)(F)F)cc1=O)c1cccc(c1)O2]

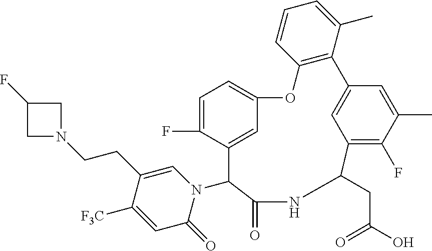 [SMILES: Cc1cc2cc(c1F)C(CC(=O)O)NC(=O)C(n1cc(CCN3CC(F)C3)c(C(F)(F)F)cc1=O)c1cc(ccc1F)Oc1cccc(C)c1-2]

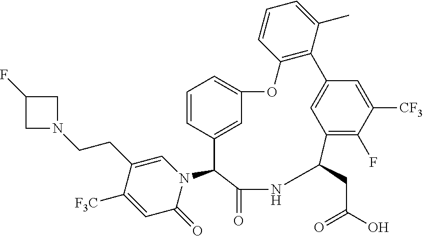 [SMILES: Cc1cccc2c1-c1cc(c(F)c(C(F)(F)F)c1)[C@@H](CC(=O)O)NC(=O)[C@@H](n1cc(CCN3CC(F)C3)c(C(F)(F)F)cc1=O)c1cccc(c1)O2]

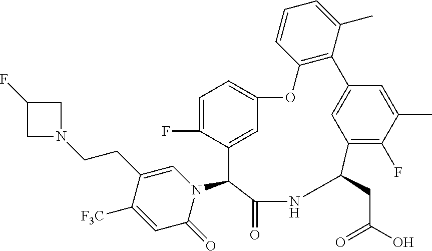 [SMILES: Cc1cc2cc(c1F)[C@@H](CC(=O)O)NC(=O)[C@@H](n1cc(CCN3CC(F)C3)c(C(F)(F)F)cc1=O)c1cc(ccc1F)Oc1cccc(C)c1-2]

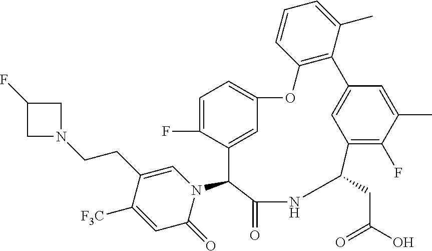 [SMILES: Cc1cc2cc(c1F)[C@H](CC(=O)O)NC(=O)[C@@H](n1cc(CCN3CC(F)C3)c(C(F)(F)F)cc1=O)c1cc(ccc1F)Oc1cccc(C)c1-2]

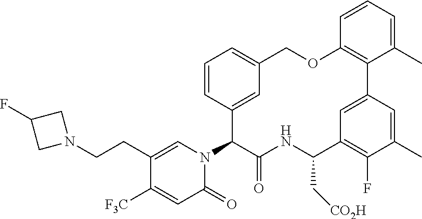 [SMILES: Cc1cc2cc(c1F)[C@H](CC(=O)O)NC(=O)[C@@H](n1cc(CCN3CC(F)C3)c(C(F)(F)F)cc1=O)c1cccc(c1)COc1cccc(C)c1-2]

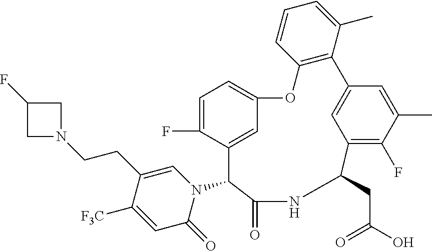 [SMILES: Cc1cc2cc(c1F)[C@@H](CC(=O)O)NC(=O)[C@H](n1cc(CCN3CC(F)C3)c(C(F)(F)F)cc1=O)c1cc(ccc1F)Oc1cccc(C)c1-2]

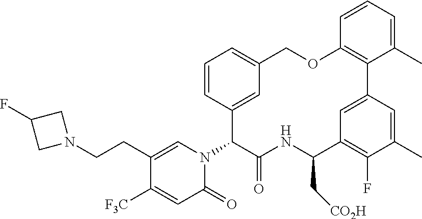 [SMILES: Cc1cc2cc(c1F)[C@@H](CC(=O)O)NC(=O)[C@H](n1cc(CCN3CC(F)C3)c(C(F)(F)F)cc1=O)c1cccc(c1)COc1cccc(C)c1-2]

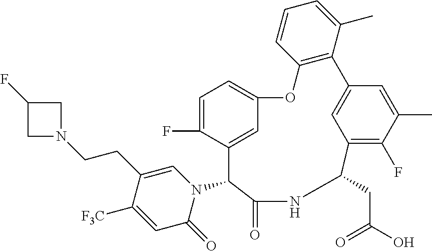 [SMILES: Cc1cc2cc(c1F)[C@H](CC(=O)O)NC(=O)[C@H](n1cc(CCN3CC(F)C3)c(C(F)(F)F)cc1=O)c1cc(ccc1F)Oc1cccc(C)c1-2]

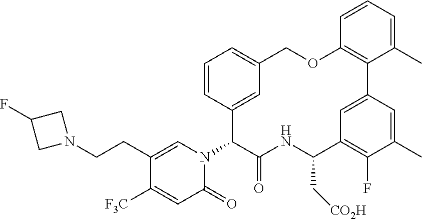 [SMILES: Cc1cc2cc(c1F)[C@H](CC(=O)O)NC(=O)[C@H](n1cc(CCN3CC(F)C3)c(C(F)(F)F)cc1=O)c1cccc(c1)COc1cccc(C)c1-2]

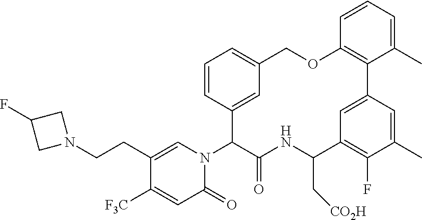 [SMILES: Cc1cc2cc(c1F)C(CC(=O)O)NC(=O)C(n1cc(CCN3CC(F)C3)c(C(F)(F)F)cc1=O)c1cccc(c1)COc1cccc(C)c1-2]

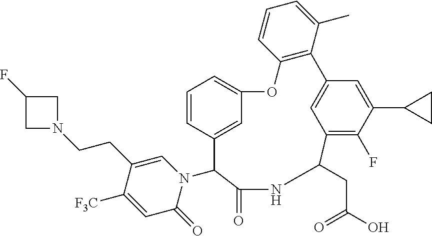 [SMILES: Cc1cccc2c1-c1cc(C3CC3)c(F)c(c1)C(CC(=O)O)NC(=O)C(n1cc(CCN3CC(F)C3)c(C(F)(F)F)cc1=O)c1cccc(c1)O2]

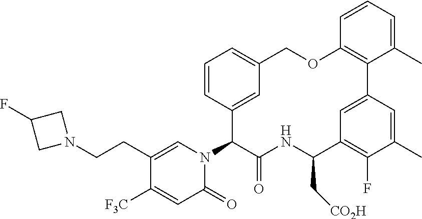 [SMILES: Cc1cc2cc(c1F)[C@@H](CC(=O)O)NC(=O)[C@@H](n1cc(CCN3CC(F)C3)c(C(F)(F)F)cc1=O)c1cccc(c1)COc1cccc(C)c1-2]

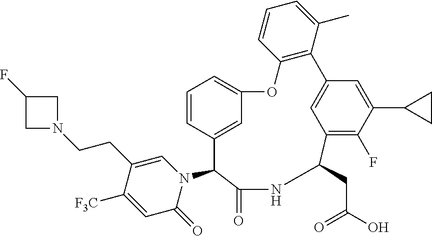 [SMILES: Cc1cccc2c1-c1cc(C3CC3)c(F)c(c1)[C@@H](CC(=O)O)NC(=O)[C@@H](n1cc(CCN3CC(F)C3)c(C(F)(F)F)cc1=O)c1cccc(c1)O2]

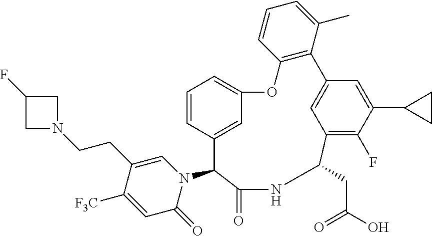 [SMILES: Cc1cccc2c1-c1cc(C3CC3)c(F)c(c1)[C@H](CC(=O)O)NC(=O)[C@@H](n1cc(CCN3CC(F)C3)c(C(F)(F)F)cc1=O)c1cccc(c1)O2]

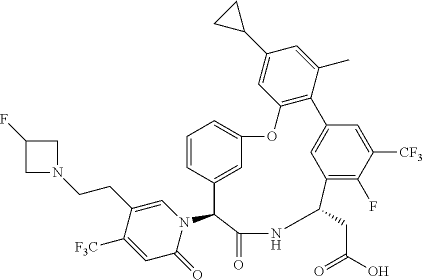 [SMILES: Cc1cc(C2CC2)cc2c1-c1cc(c(F)c(C(F)(F)F)c1)[C@H](CC(=O)O)NC(=O)[C@@H](n1cc(CCN3CC(F)C3)c(C(F)(F)F)cc1=O)c1cccc(c1)O2]

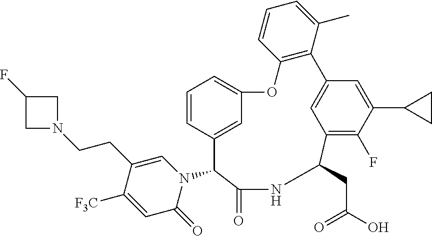 [SMILES: Cc1cccc2c1-c1cc(C3CC3)c(F)c(c1)[C@@H](CC(=O)O)NC(=O)[C@H](n1cc(CCN3CC(F)C3)c(C(F)(F)F)cc1=O)c1cccc(c1)O2]

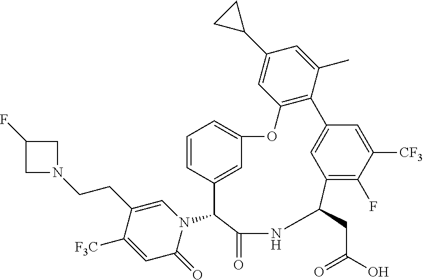 [SMILES: Cc1cc(C2CC2)cc2c1-c1cc(c(F)c(C(F)(F)F)c1)[C@@H](CC(=O)O)NC(=O)[C@H](n1cc(CCN3CC(F)C3)c(C(F)(F)F)cc1=O)c1cccc(c1)O2]

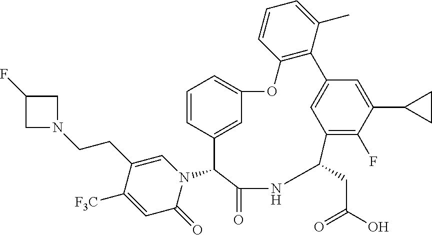 [SMILES: Cc1cccc2c1-c1cc(C3CC3)c(F)c(c1)[C@H](CC(=O)O)NC(=O)[C@H](n1cc(CCN3CC(F)C3)c(C(F)(F)F)cc1=O)c1cccc(c1)O2]

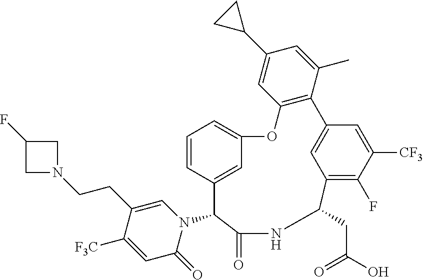 [SMILES: Cc1cc(C2CC2)cc2c1-c1cc(c(F)c(C(F)(F)F)c1)[C@H](CC(=O)O)NC(=O)[C@H](n1cc(CCN3CC(F)C3)c(C(F)(F)F)cc1=O)c1cccc(c1)O2]

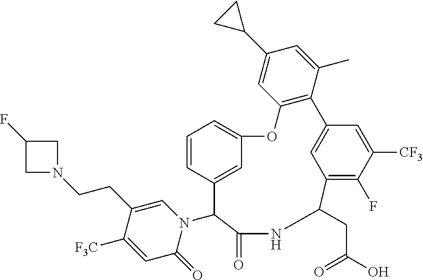 [SMILES: Cc1cc(C2CC2)cc2c1-c1cc(c(F)c(C(F)(F)F)c1)C(CC(=O)O)NC(=O)C(n1cc(CCN3CC(F)C3)c(C(F)(F)F)cc1=O)c1cccc(c1)O2]

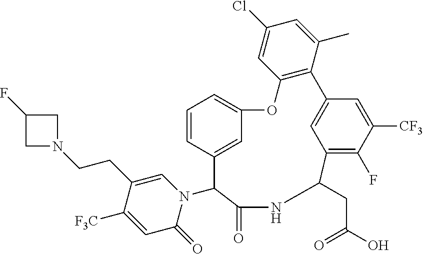 [SMILES: Cc1cc(Cl)cc2c1-c1cc(c(F)c(C(F)(F)F)c1)C(CC(=O)O)NC(=O)C(n1cc(CCN3CC(F)C3)c(C(F)(F)F)cc1=O)c1cccc(c1)O2]

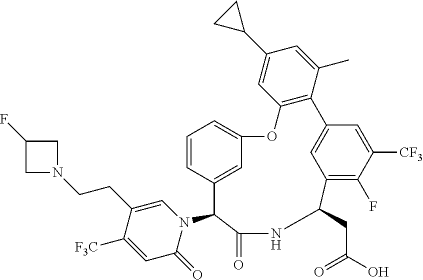 [SMILES: Cc1cc(C2CC2)cc2c1-c1cc(c(F)c(C(F)(F)F)c1)[C@@H](CC(=O)O)NC(=O)[C@@H](n1cc(CCN3CC(F)C3)c(C(F)(F)F)cc1=O)c1cccc(c1)O2]

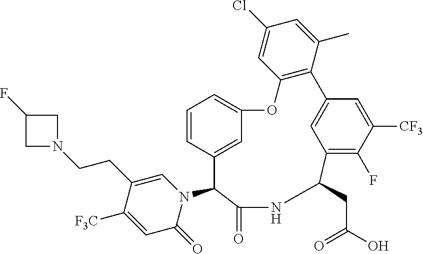 [SMILES: Cc1cc(Cl)cc2c1-c1cc(c(F)c(C(F)(F)F)c1)[C@@H](CC(=O)O)NC(=O)[C@@H](n1cc(CCN3CC(F)C3)c(C(F)(F)F)cc1=O)c1cccc(c1)O2]

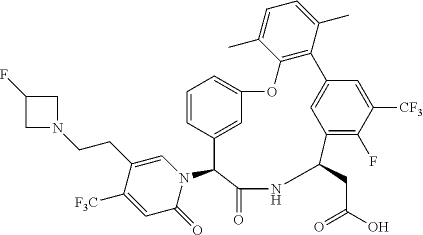 [SMILES: Cc1ccc(C)c2c1Oc1cccc(c1)[C@H](n1cc(CCN3CC(F)C3)c(C(F)(F)F)cc1=O)C(=O)N[C@H](CC(=O)O)c1cc-2cc(C(F)(F)F)c1F]

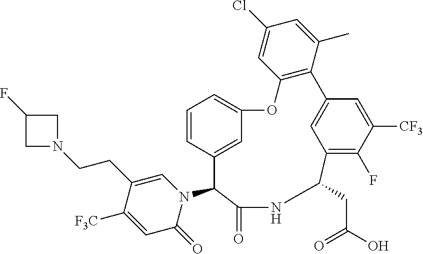 [SMILES: Cc1cc(Cl)cc2c1-c1cc(c(F)c(C(F)(F)F)c1)[C@H](CC(=O)O)NC(=O)[C@@H](n1cc(CCN3CC(F)C3)c(C(F)(F)F)cc1=O)c1cccc(c1)O2]

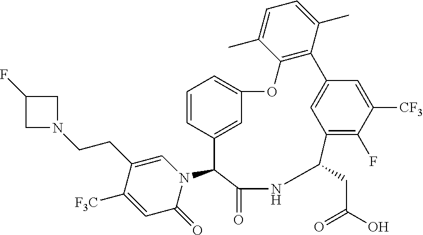 [SMILES: Cc1ccc(C)c2c1Oc1cccc(c1)[C@H](n1cc(CCN3CC(F)C3)c(C(F)(F)F)cc1=O)C(=O)N[C@@H](CC(=O)O)c1cc-2cc(C(F)(F)F)c1F]

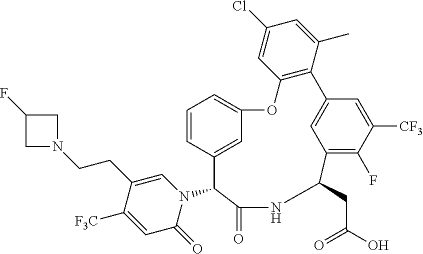 [SMILES: Cc1cc(Cl)cc2c1-c1cc(c(F)c(C(F)(F)F)c1)[C@@H](CC(=O)O)NC(=O)[C@H](n1cc(CCN3CC(F)C3)c(C(F)(F)F)cc1=O)c1cccc(c1)O2]

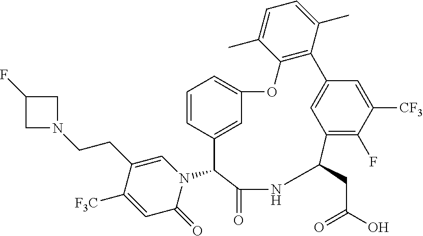 [SMILES: Cc1ccc(C)c2c1Oc1cccc(c1)[C@@H](n1cc(CCN3CC(F)C3)c(C(F)(F)F)cc1=O)C(=O)N[C@H](CC(=O)O)c1cc-2cc(C(F)(F)F)c1F]

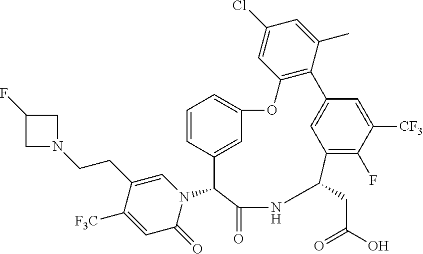 [SMILES: Cc1cc(Cl)cc2c1-c1cc(c(F)c(C(F)(F)F)c1)[C@H](CC(=O)O)NC(=O)[C@H](n1cc(CCN3CC(F)C3)c(C(F)(F)F)cc1=O)c1cccc(c1)O2]

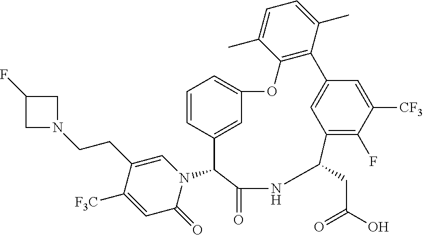 [SMILES: Cc1ccc(C)c2c1Oc1cccc(c1)[C@@H](n1cc(CCN3CC(F)C3)c(C(F)(F)F)cc1=O)C(=O)N[C@@H](CC(=O)O)c1cc-2cc(C(F)(F)F)c1F]

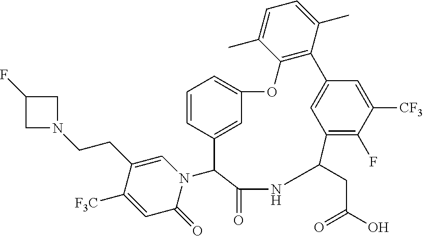 [SMILES: Cc1ccc(C)c2c1Oc1cccc(c1)C(n1cc(CCN3CC(F)C3)c(C(F)(F)F)cc1=O)C(=O)NC(CC(=O)O)c1cc-2cc(C(F)(F)F)c1F]

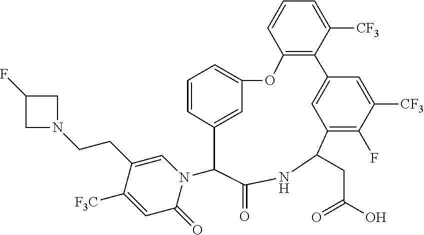 [SMILES: O=C(O)CC1NC(=O)C(n2cc(CCN3CC(F)C3)c(C(F)(F)F)cc2=O)c2cccc(c2)Oc2cccc(C(F)(F)F)c2-c2cc1c(F)c(C(F)(F)F)c2]

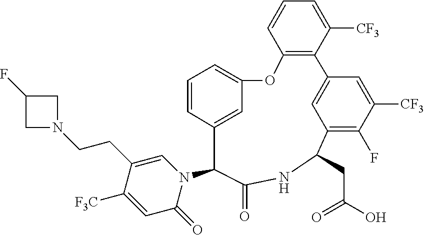 [SMILES: O=C(O)C[C@H]1NC(=O)[C@@H](n2cc(CCN3CC(F)C3)c(C(F)(F)F)cc2=O)c2cccc(c2)Oc2cccc(C(F)(F)F)c2-c2cc1c(F)c(C(F)(F)F)c2]

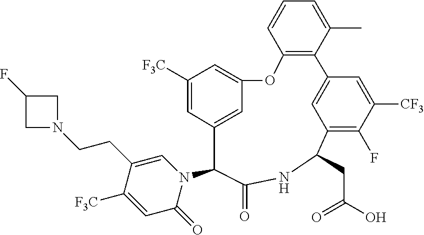 [SMILES: Cc1cccc2c1-c1cc(c(F)c(C(F)(F)F)c1)[C@@H](CC(=O)O)NC(=O)[C@@H](n1cc(CCN3CC(F)C3)c(C(F)(F)F)cc1=O)c1cc(cc(C(F)(F)F)c1)O2]

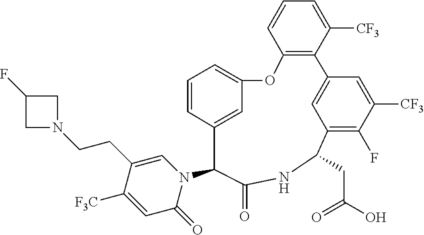 [SMILES: O=C(O)C[C@@H]1NC(=O)[C@@H](n2cc(CCN3CC(F)C3)c(C(F)(F)F)cc2=O)c2cccc(c2)Oc2cccc(C(F)(F)F)c2-c2cc1c(F)c(C(F)(F)F)c2]

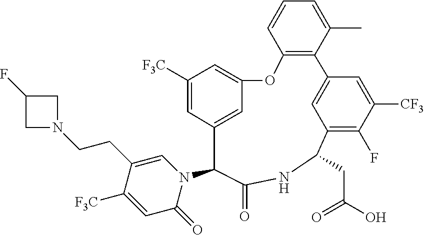 [SMILES: Cc1cccc2c1-c1cc(c(F)c(C(F)(F)F)c1)[C@H](CC(=O)O)NC(=O)[C@@H](n1cc(CCN3CC(F)C3)c(C(F)(F)F)cc1=O)c1cc(cc(C(F)(F)F)c1)O2]

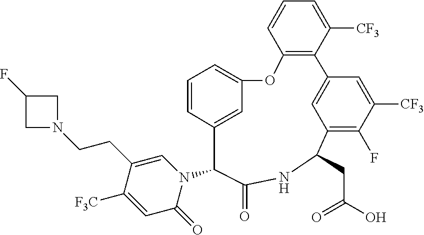 [SMILES: O=C(O)C[C@H]1NC(=O)[C@H](n2cc(CCN3CC(F)C3)c(C(F)(F)F)cc2=O)c2cccc(c2)Oc2cccc(C(F)(F)F)c2-c2cc1c(F)c(C(F)(F)F)c2]

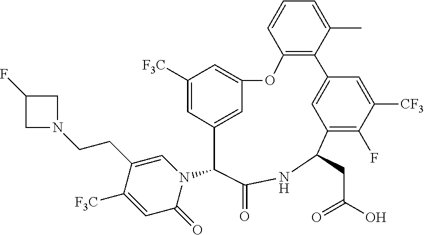 [SMILES: Cc1cccc2c1-c1cc(c(F)c(C(F)(F)F)c1)[C@@H](CC(=O)O)NC(=O)[C@H](n1cc(CCN3CC(F)C3)c(C(F)(F)F)cc1=O)c1cc(cc(C(F)(F)F)c1)O2]

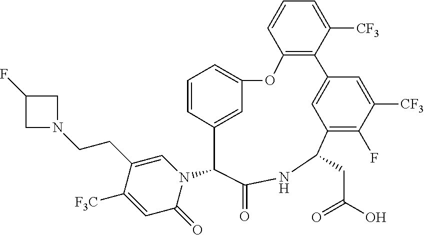 [SMILES: O=C(O)C[C@@H]1NC(=O)[C@H](n2cc(CCN3CC(F)C3)c(C(F)(F)F)cc2=O)c2cccc(c2)Oc2cccc(C(F)(F)F)c2-c2cc1c(F)c(C(F)(F)F)c2]

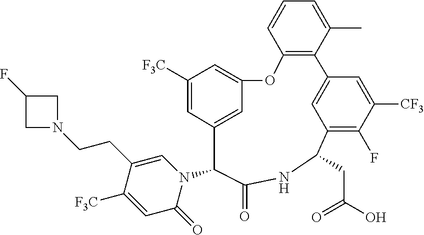 [SMILES: Cc1cccc2c1-c1cc(c(F)c(C(F)(F)F)c1)[C@H](CC(=O)O)NC(=O)[C@H](n1cc(CCN3CC(F)C3)c(C(F)(F)F)cc1=O)c1cc(cc(C(F)(F)F)c1)O2]

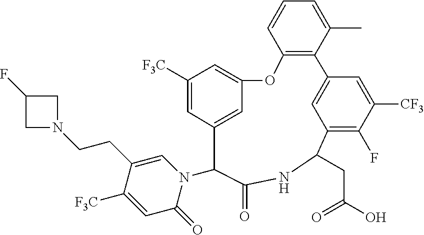 [SMILES: Cc1cccc2c1-c1cc(c(F)c(C(F)(F)F)c1)C(CC(=O)O)NC(=O)C(n1cc(CCN3CC(F)C3)c(C(F)(F)F)cc1=O)c1cc(cc(C(F)(F)F)c1)O2]

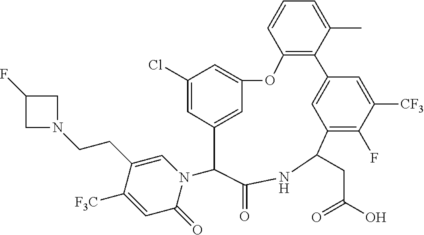 [SMILES: Cc1cccc2c1-c1cc(c(F)c(C(F)(F)F)c1)C(CC(=O)O)NC(=O)C(n1cc(CCN3CC(F)C3)c(C(F)(F)F)cc1=O)c1cc(Cl)cc(c1)O2]

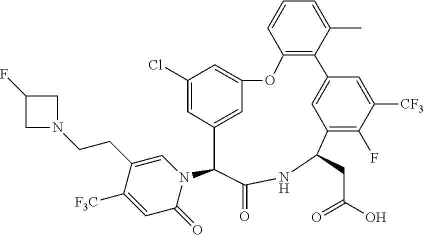 [SMILES: Cc1cccc2c1-c1cc(c(F)c(C(F)(F)F)c1)[C@@H](CC(=O)O)NC(=O)[C@@H](n1cc(CCN3CC(F)C3)c(C(F)(F)F)cc1=O)c1cc(Cl)cc(c1)O2]

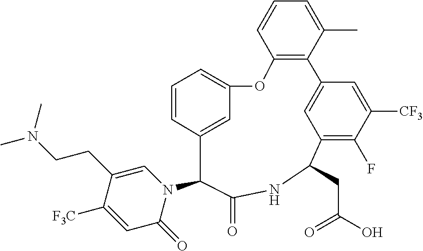 [SMILES: Cc1cccc2c1-c1cc(c(F)c(C(F)(F)F)c1)[C@@H](CC(=O)O)NC(=O)[C@@H](n1cc(CCN(C)C)c(C(F)(F)F)cc1=O)c1cccc(c1)O2]

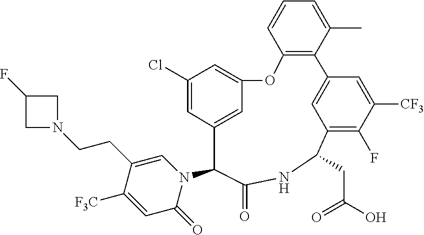 [SMILES: Cc1cccc2c1-c1cc(c(F)c(C(F)(F)F)c1)[C@H](CC(=O)O)NC(=O)[C@@H](n1cc(CCN3CC(F)C3)c(C(F)(F)F)cc1=O)c1cc(Cl)cc(c1)O2]

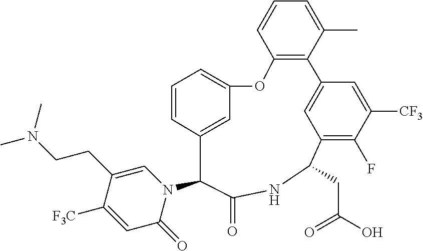 [SMILES: Cc1cccc2c1-c1cc(c(F)c(C(F)(F)F)c1)[C@H](CC(=O)O)NC(=O)[C@@H](n1cc(CCN(C)C)c(C(F)(F)F)cc1=O)c1cccc(c1)O2]

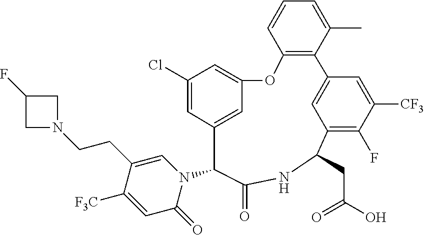 [SMILES: Cc1cccc2c1-c1cc(c(F)c(C(F)(F)F)c1)[C@@H](CC(=O)O)NC(=O)[C@H](n1cc(CCN3CC(F)C3)c(C(F)(F)F)cc1=O)c1cc(Cl)cc(c1)O2]

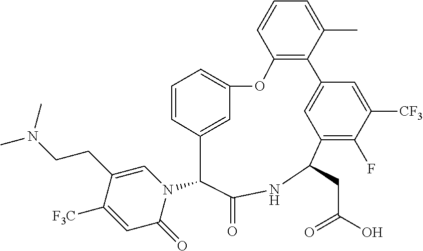 [SMILES: Cc1cccc2c1-c1cc(c(F)c(C(F)(F)F)c1)[C@@H](CC(=O)O)NC(=O)[C@H](n1cc(CCN(C)C)c(C(F)(F)F)cc1=O)c1cccc(c1)O2]

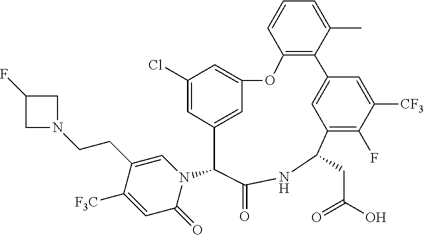 [SMILES: Cc1cccc2c1-c1cc(c(F)c(C(F)(F)F)c1)[C@H](CC(=O)O)NC(=O)[C@H](n1cc(CCN3CC(F)C3)c(C(F)(F)F)cc1=O)c1cc(Cl)cc(c1)O2]

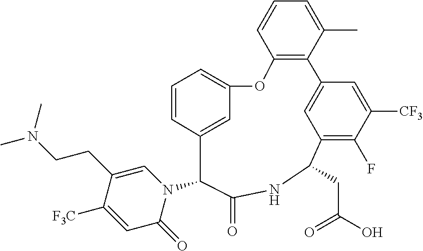 [SMILES: Cc1cccc2c1-c1cc(c(F)c(C(F)(F)F)c1)[C@H](CC(=O)O)NC(=O)[C@H](n1cc(CCN(C)C)c(C(F)(F)F)cc1=O)c1cccc(c1)O2]

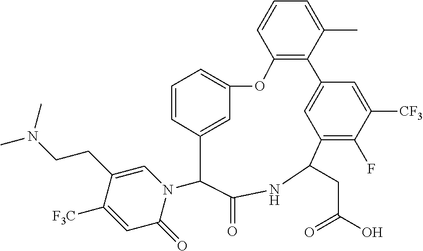 [SMILES: Cc1cccc2c1-c1cc(c(F)c(C(F)(F)F)c1)C(CC(=O)O)NC(=O)C(n1cc(CCN(C)C)c(C(F)(F)F)cc1=O)c1cccc(c1)O2]

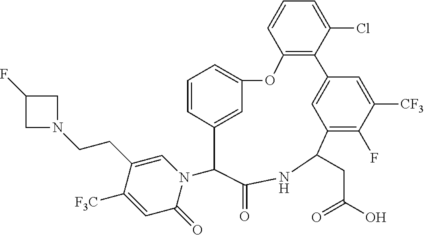 [SMILES: O=C(O)CC1NC(=O)C(n2cc(CCN3CC(F)C3)c(C(F)(F)F)cc2=O)c2cccc(c2)Oc2cccc(Cl)c2-c2cc1c(F)c(C(F)(F)F)c2]

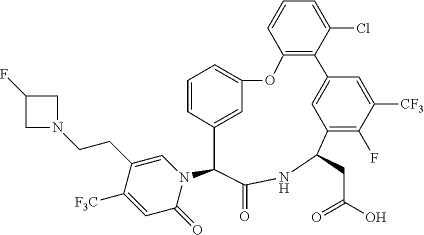 [SMILES: O=C(O)C[C@H]1NC(=O)[C@@H](n2cc(CCN3CC(F)C3)c(C(F)(F)F)cc2=O)c2cccc(c2)Oc2cccc(Cl)c2-c2cc1c(F)c(C(F)(F)F)c2]

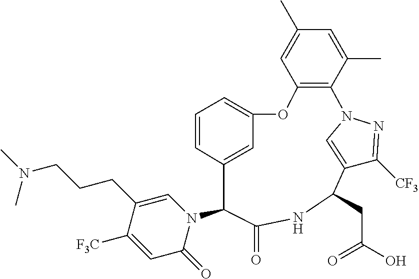 [SMILES: Cc1cc(C)c2c(c1)Oc1cccc(c1)[C@H](n1cc(CCCN(C)C)c(C(F)(F)F)cc1=O)C(=O)N[C@H](CC(=O)O)c1cn-2nc1C(F)(F)F]

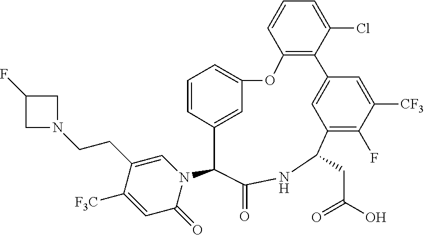 [SMILES: O=C(O)C[C@@H]1NC(=O)[C@@H](n2cc(CCN3CC(F)C3)c(C(F)(F)F)cc2=O)c2cccc(c2)Oc2cccc(Cl)c2-c2cc1c(F)c(C(F)(F)F)c2]

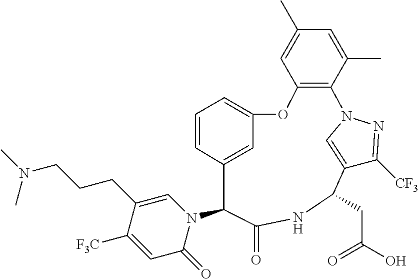 [SMILES: Cc1cc(C)c2c(c1)Oc1cccc(c1)[C@H](n1cc(CCCN(C)C)c(C(F)(F)F)cc1=O)C(=O)N[C@@H](CC(=O)O)c1cn-2nc1C(F)(F)F]

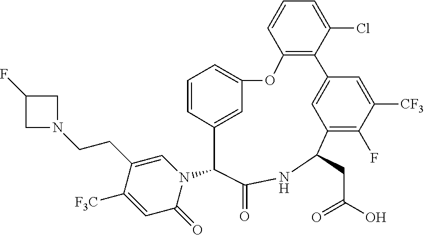 [SMILES: O=C(O)C[C@H]1NC(=O)[C@H](n2cc(CCN3CC(F)C3)c(C(F)(F)F)cc2=O)c2cccc(c2)Oc2cccc(Cl)c2-c2cc1c(F)c(C(F)(F)F)c2]

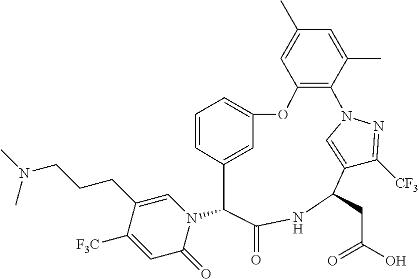 [SMILES: Cc1cc(C)c2c(c1)Oc1cccc(c1)[C@@H](n1cc(CCCN(C)C)c(C(F)(F)F)cc1=O)C(=O)N[C@H](CC(=O)O)c1cn-2nc1C(F)(F)F]

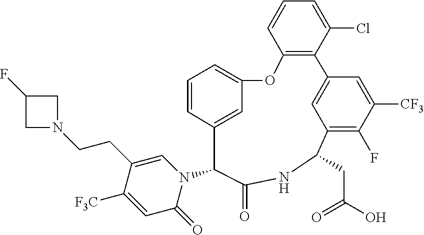 [SMILES: O=C(O)C[C@@H]1NC(=O)[C@H](n2cc(CCN3CC(F)C3)c(C(F)(F)F)cc2=O)c2cccc(c2)Oc2cccc(Cl)c2-c2cc1c(F)c(C(F)(F)F)c2]

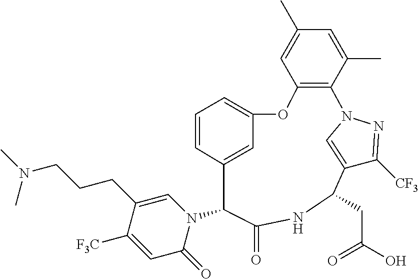 [SMILES: Cc1cc(C)c2c(c1)Oc1cccc(c1)[C@@H](n1cc(CCCN(C)C)c(C(F)(F)F)cc1=O)C(=O)N[C@@H](CC(=O)O)c1cn-2nc1C(F)(F)F]

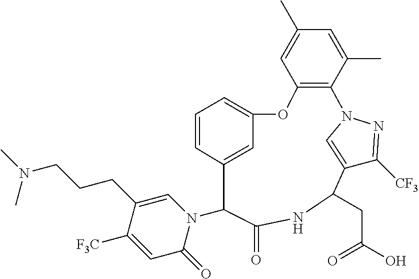 [SMILES: Cc1cc(C)c2c(c1)Oc1cccc(c1)C(n1cc(CCCN(C)C)c(C(F)(F)F)cc1=O)C(=O)NC(CC(=O)O)c1cn-2nc1C(F)(F)F]

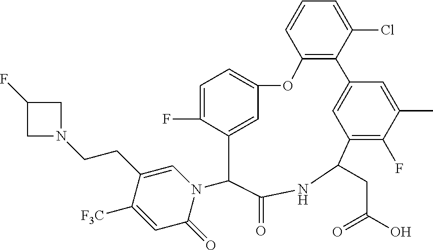 [SMILES: Cc1cc2cc(c1F)C(CC(=O)O)NC(=O)C(n1cc(CCN3CC(F)C3)c(C(F)(F)F)cc1=O)c1cc(ccc1F)Oc1cccc(Cl)c1-2]

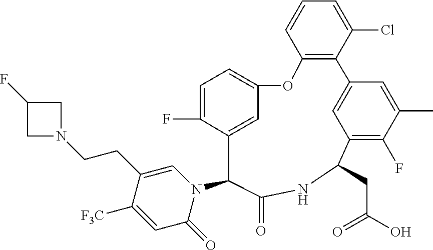 [SMILES: Cc1cc2cc(c1F)[C@@H](CC(=O)O)NC(=O)[C@@H](n1cc(CCN3CC(F)C3)c(C(F)(F)F)cc1=O)c1cc(ccc1F)Oc1cccc(Cl)c1-2]

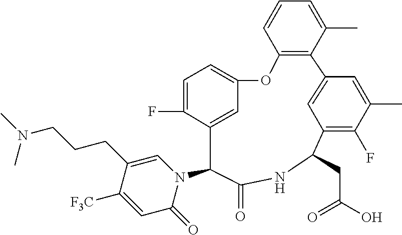

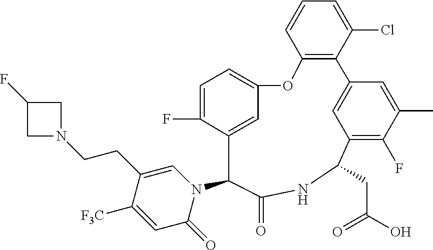 [SMILES: Cc1cc2cc(c1F)[C@H](CC(=O)O)NC(=O)[C@@H](n1cc(CCN3CC(F)C3)c(C(F)(F)F)cc1=O)c1cc(ccc1F)Oc1cccc(Cl)c1-2]

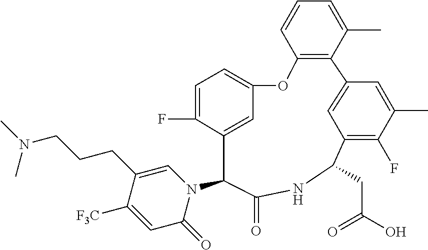

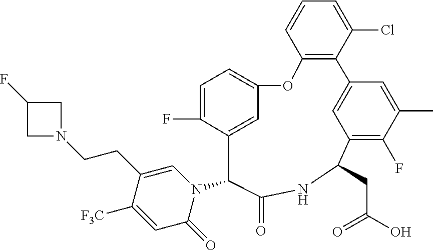 [SMILES: Cc1cc2cc(c1F)[C@@H](CC(=O)O)NC(=O)[C@H](n1cc(CCN3CC(F)C3)c(C(F)(F)F)cc1=O)c1cc(ccc1F)Oc1cccc(Cl)c1-2]

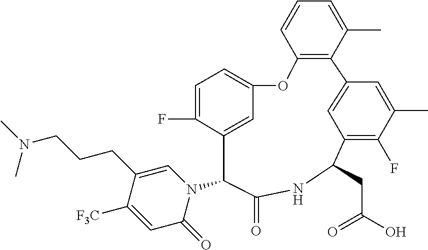

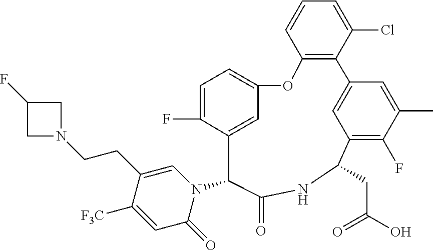 [SMILES: Cc1cc2cc(c1F)[C@H](CC(=O)O)NC(=O)[C@H](n1cc(CCN3CC(F)C3)c(C(F)(F)F)cc1=O)c1cc(ccc1F)Oc1cccc(Cl)c1-2]

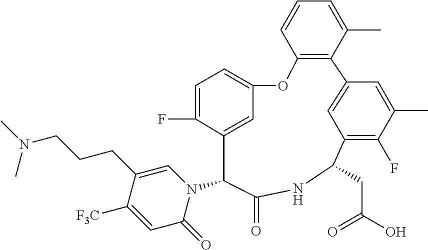

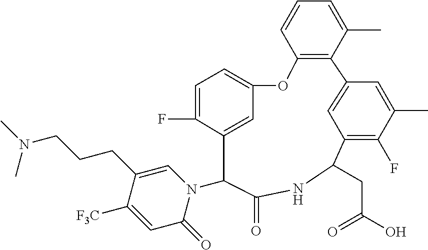

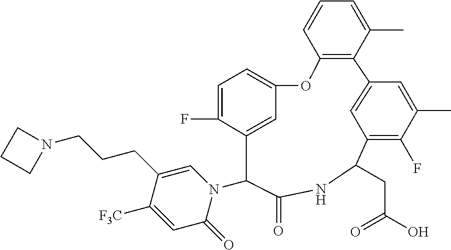 [SMILES: Cc1cc2cc(c1F)C(CC(=O)O)NC(=O)C(n1cc(CCCN3CCC3)c(C(F)(F)F)cc1=O)c1cc(ccc1F)Oc1cccc(C)c1-2]

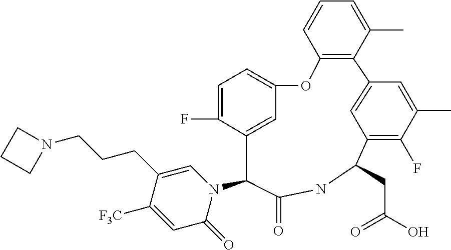 [SMILES: Cc1cc2cc(c1F)[C@@H](CC(=O)O)NC(=O)[C@@H](n1cc(CCCN3CCC3)c(C(F)(F)F)cc1=O)c1cc(ccc1F)Oc1cccc(C)c1-2]

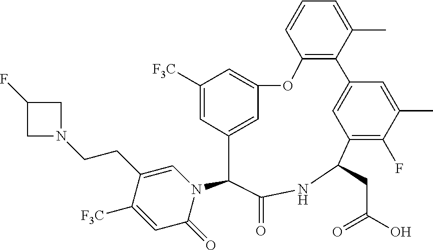 [SMILES: Cc1cc2cc(c1F)[C@@H](CC(=O)O)NC(=O)[C@@H](n1cc(CCN3CC(F)C3)c(C(F)(F)F)cc1=O)c1cc(cc(C(F)(F)F)c1)Oc1cccc(C)c1-2]

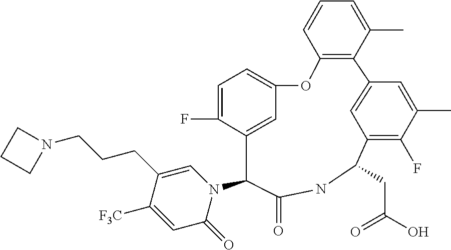 [SMILES: Cc1cc2cc(c1F)[C@H](CC(=O)O)NC(=O)[C@@H](n1cc(CCCN3CCC3)c(C(F)(F)F)cc1=O)c1cc(ccc1F)Oc1cccc(C)c1-2]

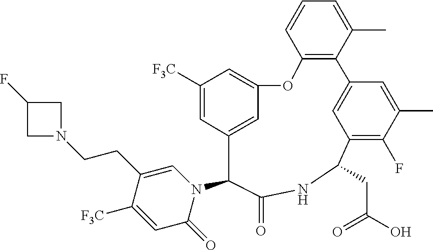 [SMILES: Cc1cc2cc(c1F)[C@H](CC(=O)O)NC(=O)[C@@H](n1cc(CCN3CC(F)C3)c(C(F)(F)F)cc1=O)c1cc(cc(C(F)(F)F)c1)Oc1cccc(C)c1-2]

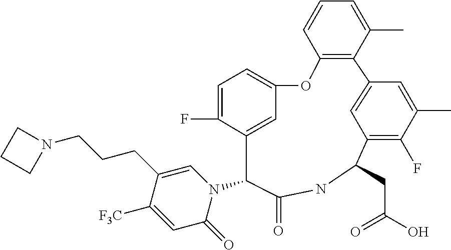 [SMILES: Cc1cc2cc(c1F)[C@@H](CC(=O)O)NC(=O)[C@H](n1cc(CCCN3CCC3)c(C(F)(F)F)cc1=O)c1cc(ccc1F)Oc1cccc(C)c1-2]

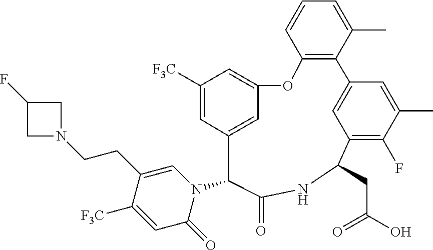 [SMILES: Cc1cc2cc(c1F)[C@@H](CC(=O)O)NC(=O)[C@H](n1cc(CCN3CC(F)C3)c(C(F)(F)F)cc1=O)c1cc(cc(C(F)(F)F)c1)Oc1cccc(C)c1-2]

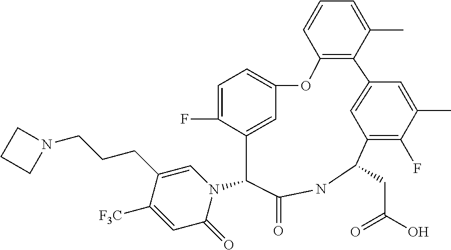 [SMILES: Cc1cc2cc(c1F)[C@H](CC(=O)O)NC(=O)[C@H](n1cc(CCCN3CCC3)c(C(F)(F)F)cc1=O)c1cc(ccc1F)Oc1cccc(C)c1-2]

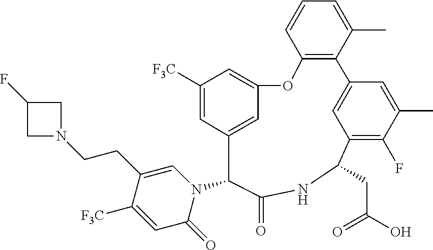 [SMILES: Cc1cc2cc(c1F)[C@H](CC(=O)O)NC(=O)[C@H](n1cc(CCN3CC(F)C3)c(C(F)(F)F)cc1=O)c1cc(cc(C(F)(F)F)c1)Oc1cccc(C)c1-2]

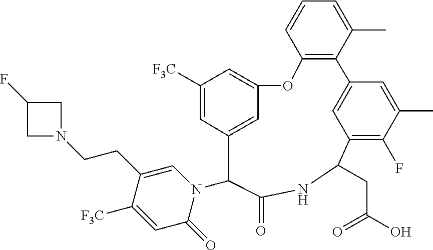 [SMILES: Cc1cc2cc(c1F)C(CC(=O)O)NC(=O)C(n1cc(CCN3CC(F)C3)c(C(F)(F)F)cc1=O)c1cc(cc(C(F)(F)F)c1)Oc1cccc(C)c1-2]

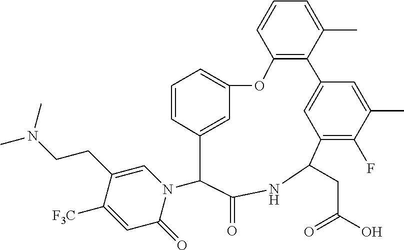

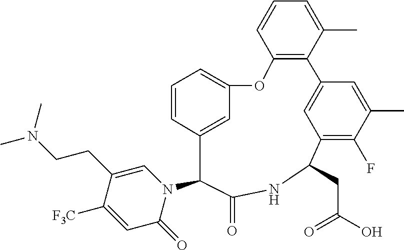

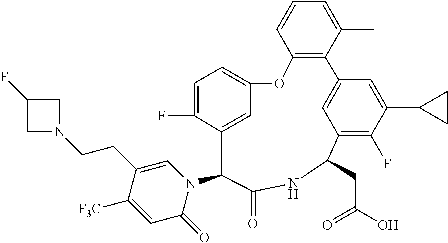 [SMILES: Cc1cccc2c1-c1cc(C3CC3)c(F)c(c1)[C@@H](CC(=O)O)NC(=O)[C@@H](n1cc(CCN3CC(F)C3)c(C(F)(F)F)cc1=O)c1cc(ccc1F)O2]

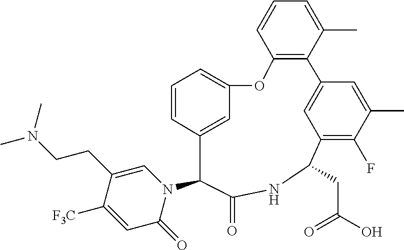

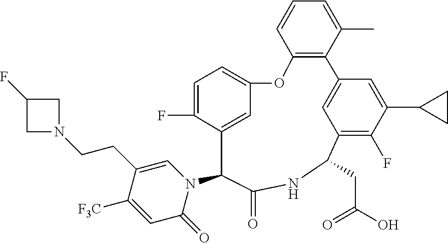 [SMILES: Cc1cccc2c1-c1cc(C3CC3)c(F)c(c1)[C@H](CC(=O)O)NC(=O)[C@@H](n1cc(CCN3CC(F)C3)c(C(F)(F)F)cc1=O)c1cc(ccc1F)O2]

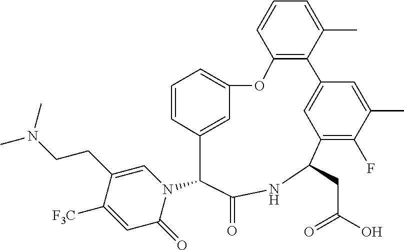

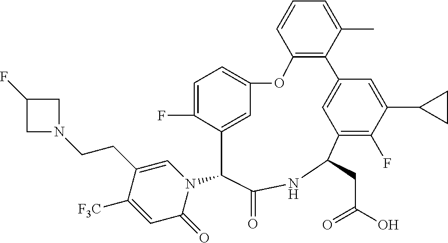 [SMILES: Cc1cccc2c1-c1cc(C3CC3)c(F)c(c1)[C@@H](CC(=O)O)NC(=O)[C@H](n1cc(CCN3CC(F)C3)c(C(F)(F)F)cc1=O)c1cc(ccc1F)O2]

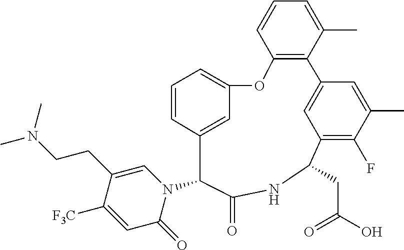

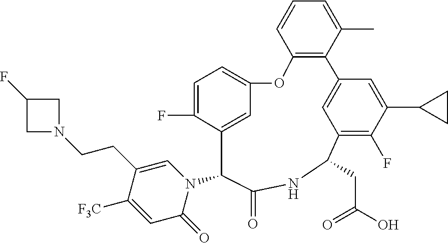 [SMILES: Cc1cccc2c1-c1cc(C3CC3)c(F)c(c1)[C@H](CC(=O)O)NC(=O)[C@H](n1cc(CCN3CC(F)C3)c(C(F)(F)F)cc1=O)c1cc(ccc1F)O2]

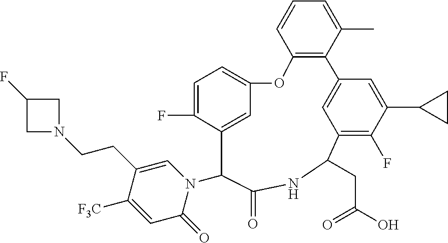 [SMILES: Cc1cccc2c1-c1cc(C3CC3)c(F)c(c1)C(CC(=O)O)NC(=O)C(n1cc(CCN3CC(F)C3)c(C(F)(F)F)cc1=O)c1cc(ccc1F)O2]

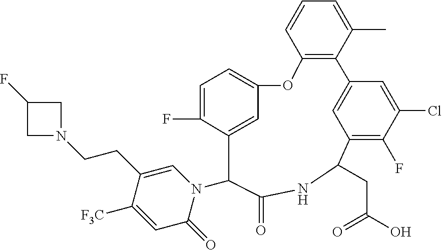 [SMILES: Cc1cccc2c1-c1cc(Cl)c(F)c(c1)C(CC(=O)O)NC(=O)C(n1cc(CCN3CC(F)C3)c(C(F)(F)F)cc1=O)c1cc(ccc1F)O2]

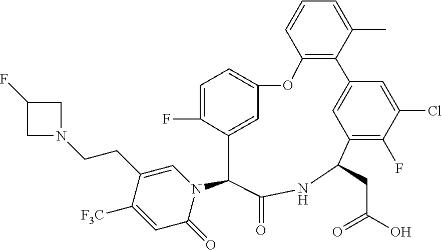 [SMILES: Cc1cccc2c1-c1cc(Cl)c(F)c(c1)[C@@H](CC(=O)O)NC(=O)[C@@H](n1cc(CCN3CC(F)C3)c(C(F)(F)F)cc1=O)c1cc(ccc1F)O2]

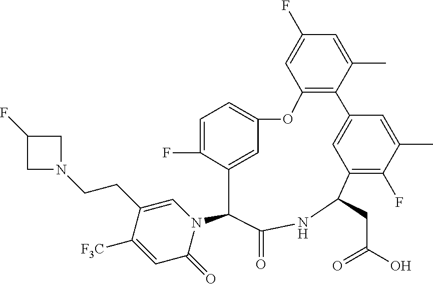 [SMILES: Cc1cc2cc(c1F)[C@@H](CC(=O)O)NC(=O)[C@@H](n1cc(CCN3CC(F)C3)c(C(F)(F)F)cc1=O)c1cc(ccc1F)Oc1cc(F)cc(C)c1-2]

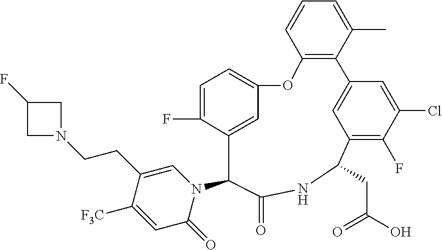 [SMILES: Cc1cccc2c1-c1cc(Cl)c(F)c(c1)[C@H](CC(=O)O)NC(=O)[C@@H](n1cc(CCN3CC(F)C3)c(C(F)(F)F)cc1=O)c1cc(ccc1F)O2]

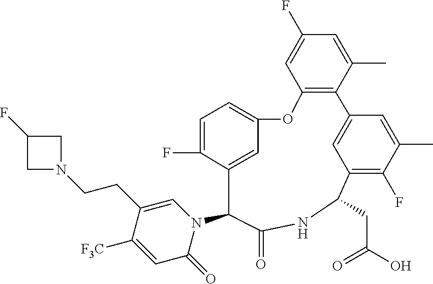 [SMILES: Cc1cc2cc(c1F)[C@H](CC(=O)O)NC(=O)[C@@H](n1cc(CCN3CC(F)C3)c(C(F)(F)F)cc1=O)c1cc(ccc1F)Oc1cc(F)cc(C)c1-2]

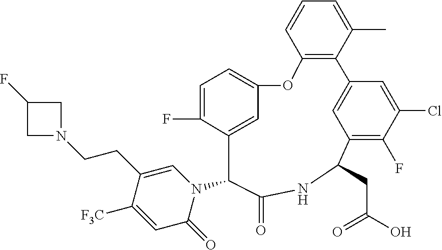 [SMILES: Cc1cccc2c1-c1cc(Cl)c(F)c(c1)[C@@H](CC(=O)O)NC(=O)[C@H](n1cc(CCN3CC(F)C3)c(C(F)(F)F)cc1=O)c1cc(ccc1F)O2]

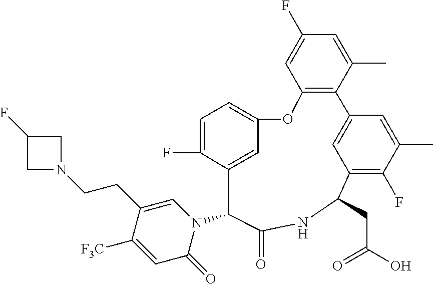 [SMILES: Cc1cc2cc(c1F)[C@@H](CC(=O)O)NC(=O)[C@H](n1cc(CCN3CC(F)C3)c(C(F)(F)F)cc1=O)c1cc(ccc1F)Oc1cc(F)cc(C)c1-2]

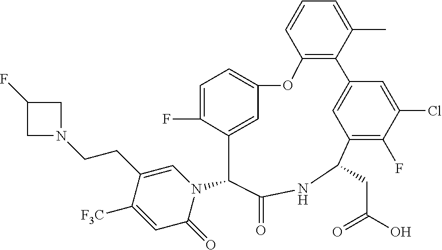 [SMILES: Cc1cccc2c1-c1cc(Cl)c(F)c(c1)[C@H](CC(=O)O)NC(=O)[C@H](n1cc(CCN3CC(F)C3)c(C(F)(F)F)cc1=O)c1cc(ccc1F)O2]

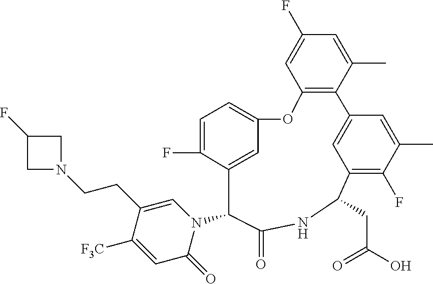 [SMILES: Cc1cc2cc(c1F)[C@H](CC(=O)O)NC(=O)[C@H](n1cc(CCN3CC(F)C3)c(C(F)(F)F)cc1=O)c1cc(ccc1F)Oc1cc(F)cc(C)c1-2]

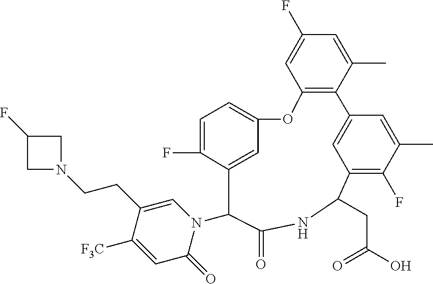 [SMILES: Cc1cc2cc(c1F)C(CC(=O)O)NC(=O)C(n1cc(CCN3CC(F)C3)c(C(F)(F)F)cc1=O)c1cc(ccc1F)Oc1cc(F)cc(C)c1-2]

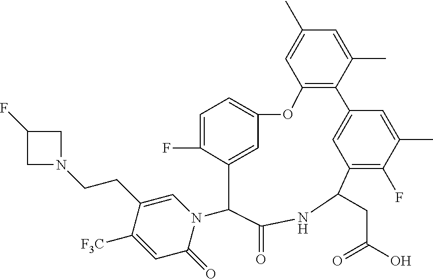 [SMILES: Cc1cc(C)c2c(c1)Oc1ccc(F)c(c1)C(n1cc(CCN3CC(F)C3)c(C(F)(F)F)cc1=O)C(=O)NC(CC(=O)O)c1cc-2cc(C)c1F]

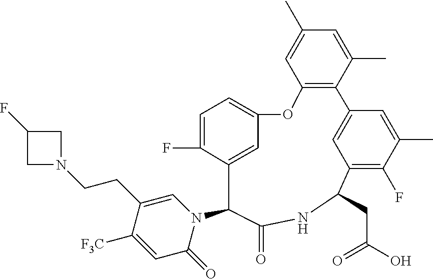 [SMILES: Cc1cc(C)c2c(c1)Oc1ccc(F)c(c1)[C@H](n1cc(CCN3CC(F)C3)c(C(F)(F)F)cc1=O)C(=O)N[C@H](CC(=O)O)c1cc-2cc(C)c1F]

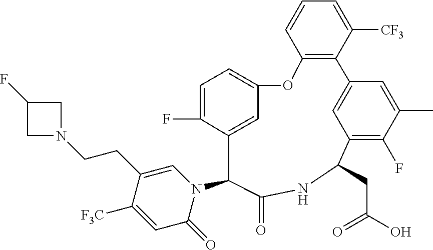 [SMILES: Cc1cc2cc(c1F)[C@@H](CC(=O)O)NC(=O)[C@@H](n1cc(CCN3CC(F)C3)c(C(F)(F)F)cc1=O)c1cc(ccc1F)Oc1cccc(C(F)(F)F)c1-2]

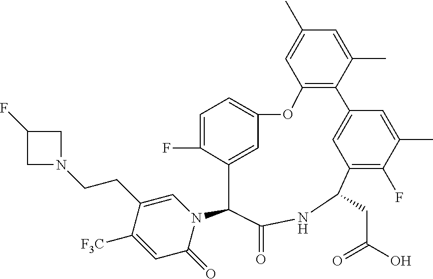 [SMILES: Cc1cc(C)c2c(c1)Oc1ccc(F)c(c1)[C@H](n1cc(CCN3CC(F)C3)c(C(F)(F)F)cc1=O)C(=O)N[C@@H](CC(=O)O)c1cc-2cc(C)c1F]

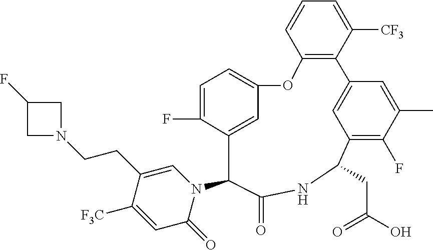 [SMILES: Cc1cc2cc(c1F)[C@H](CC(=O)O)NC(=O)[C@@H](n1cc(CCN3CC(F)C3)c(C(F)(F)F)cc1=O)c1cc(ccc1F)Oc1cccc(C(F)(F)F)c1-2]

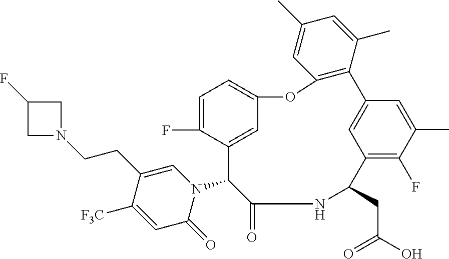 [SMILES: Cc1cc(C)c2c(c1)Oc1ccc(F)c(c1)[C@@H](n1cc(CCN3CC(F)C3)c(C(F)(F)F)cc1=O)C(=O)N[C@H](CC(=O)O)c1cc-2cc(C)c1F]

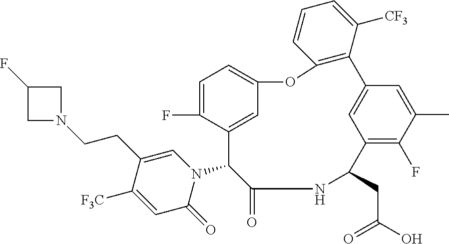 [SMILES: Cc1cc2cc(c1F)[C@@H](CC(=O)O)NC(=O)[C@H](n1cc(CCN3CC(F)C3)c(C(F)(F)F)cc1=O)c1cc(ccc1F)Oc1cccc(C(F)(F)F)c1-2]

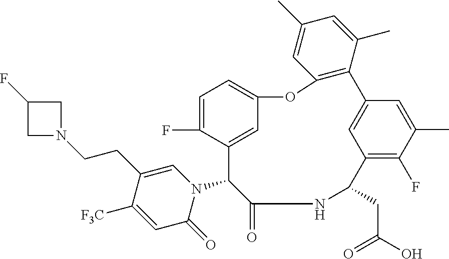 [SMILES: Cc1cc(C)c2c(c1)Oc1ccc(F)c(c1)[C@@H](n1cc(CCN3CC(F)C3)c(C(F)(F)F)cc1=O)C(=O)N[C@@H](CC(=O)O)c1cc-2cc(C)c1F]

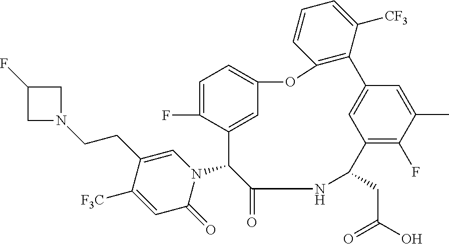 [SMILES: Cc1cc2cc(c1F)[C@H](CC(=O)O)NC(=O)[C@H](n1cc(CCN3CC(F)C3)c(C(F)(F)F)cc1=O)c1cc(ccc1F)Oc1cccc(C(F)(F)F)c1-2]

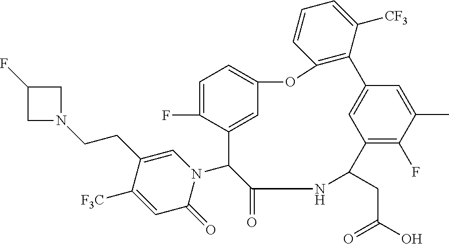 [SMILES: Cc1cc2cc(c1F)C(CC(=O)O)NC(=O)C(n1cc(CCN3CC(F)C3)c(C(F)(F)F)cc1=O)c1cc(ccc1F)Oc1cccc(C(F)(F)F)c1-2]

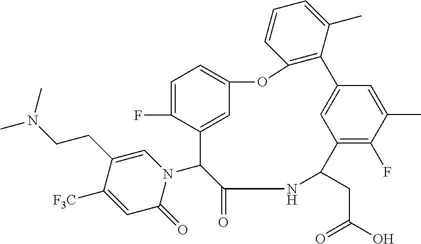

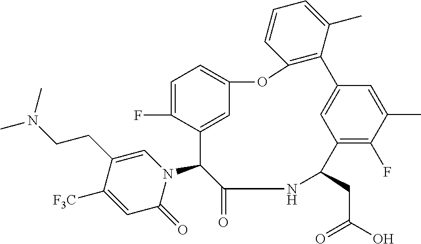

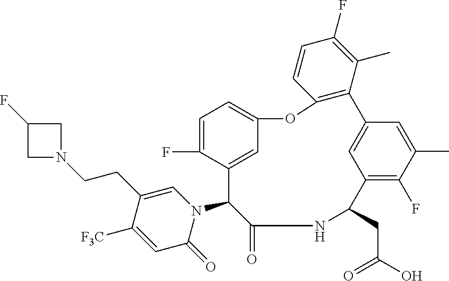 [SMILES: Cc1cc2cc(c1F)[C@@H](CC(=O)O)NC(=O)[C@@H](n1cc(CCN3CC(F)C3)c(C(F)(F)F)cc1=O)c1cc(ccc1F)Oc1ccc(F)c(C)c1-2]

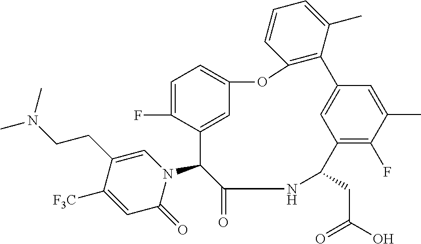

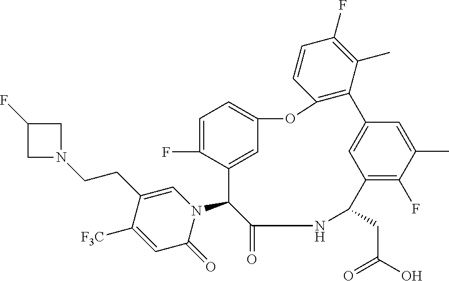 [SMILES: Cc1cc2cc(c1F)[C@H](CC(=O)O)NC(=O)[C@@H](n1cc(CCN3CC(F)C3)c(C(F)(F)F)cc1=O)c1cc(ccc1F)Oc1ccc(F)c(C)c1-2]

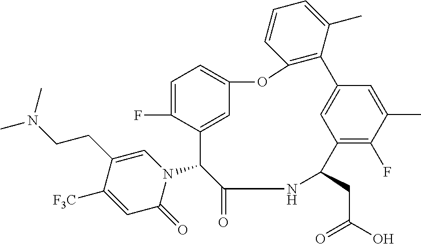

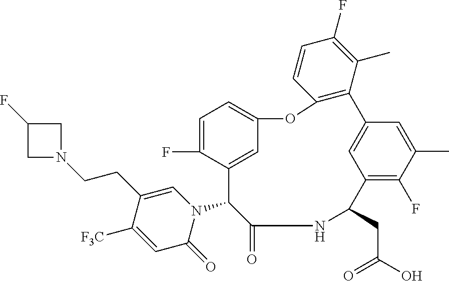 [SMILES: Cc1cc2cc(c1F)[C@@H](CC(=O)O)NC(=O)[C@H](n1cc(CCN3CC(F)C3)c(C(F)(F)F)cc1=O)c1cc(ccc1F)Oc1ccc(F)c(C)c1-2]

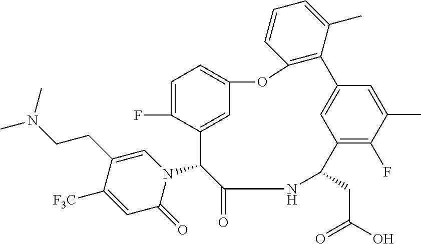

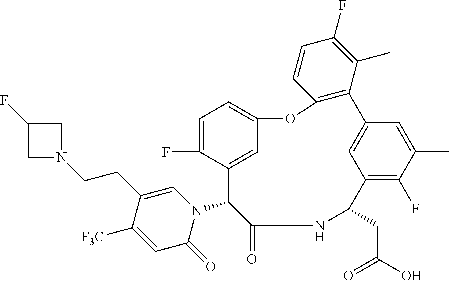 [SMILES: Cc1cc2cc(c1F)[C@H](CC(=O)O)NC(=O)[C@H](n1cc(CCN3CC(F)C3)c(C(F)(F)F)cc1=O)c1cc(ccc1F)Oc1ccc(F)c(C)c1-2]

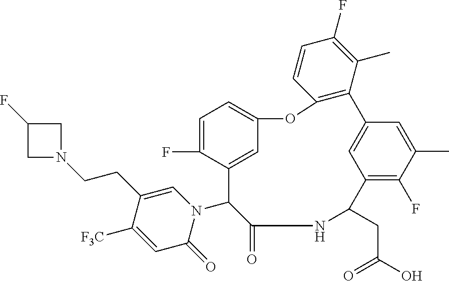 [SMILES: Cc1cc2cc(c1F)C(CC(=O)O)NC(=O)C(n1cc(CCN3CC(F)C3)c(C(F)(F)F)cc1=O)c1cc(ccc1F)Oc1ccc(F)c(C)c1-2]

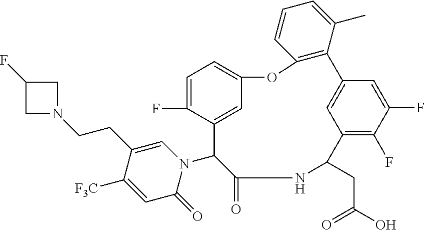 [SMILES: Cc1cccc2c1-c1cc(F)c(F)c(c1)C(CC(=O)O)NC(=O)C(n1cc(CCN3CC(F)C3)c(C(F)(F)F)cc1=O)c1cc(ccc1F)O2]

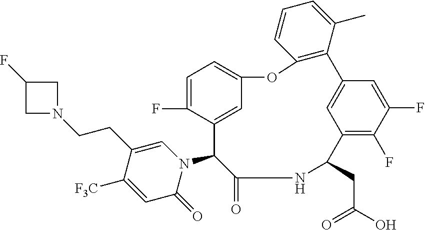 [SMILES: Cc1cccc2c1-c1cc(F)c(F)c(c1)[C@@H](CC(=O)O)NC(=O)[C@@H](n1cc(CCN3CC(F)C3)c(C(F)(F)F)cc1=O)c1cc(ccc1F)O2]

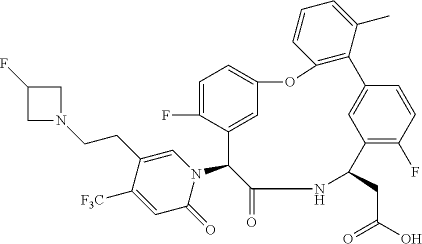 [SMILES: Cc1cccc2c1-c1ccc(F)c(c1)[C@@H](CC(=O)O)NC(=O)[C@@H](n1cc(CCN3CC(F)C3)c(C(F)(F)F)cc1=O)c1cc(ccc1F)O2]

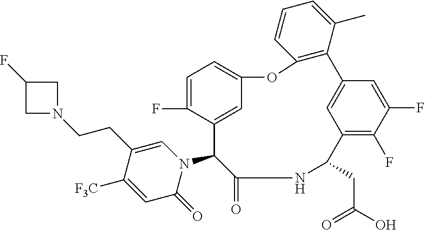 [SMILES: Cc1cccc2c1-c1cc(F)c(F)c(c1)[C@H](CC(=O)O)NC(=O)[C@@H](n1cc(CCN3CC(F)C3)c(C(F)(F)F)cc1=O)c1cc(ccc1F)O2]

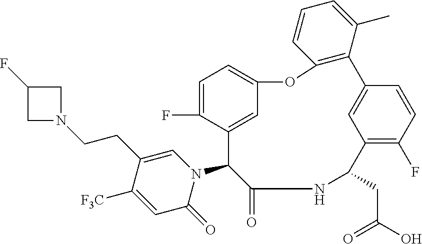 [SMILES: Cc1cccc2c1-c1ccc(F)c(c1)[C@H](CC(=O)O)NC(=O)[C@@H](n1cc(CCN3CC(F)C3)c(C(F)(F)F)cc1=O)c1cc(ccc1F)O2]

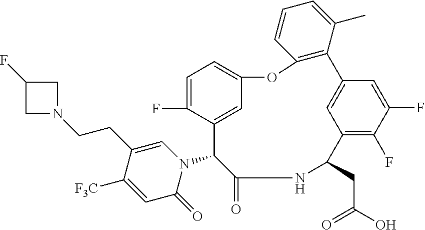 [SMILES: Cc1cccc2c1-c1cc(F)c(F)c(c1)[C@@H](CC(=O)O)NC(=O)[C@H](n1cc(CCN3CC(F)C3)c(C(F)(F)F)cc1=O)c1cc(ccc1F)O2]

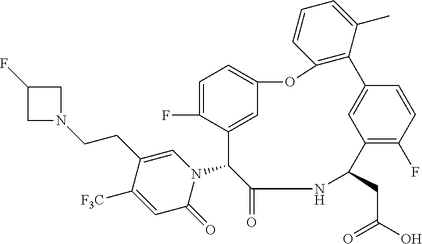 [SMILES: Cc1cccc2c1-c1ccc(F)c(c1)[C@@H](CC(=O)O)NC(=O)[C@H](n1cc(CCN3CC(F)C3)c(C(F)(F)F)cc1=O)c1cc(ccc1F)O2]

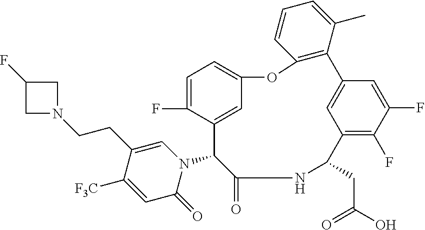 [SMILES: Cc1cccc2c1-c1cc(F)c(F)c(c1)[C@H](CC(=O)O)NC(=O)[C@H](n1cc(CCN3CC(F)C3)c(C(F)(F)F)cc1=O)c1cc(ccc1F)O2]

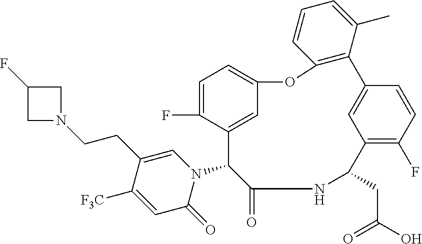 [SMILES: Cc1cccc2c1-c1ccc(F)c(c1)[C@H](CC(=O)O)NC(=O)[C@H](n1cc(CCN3CC(F)C3)c(C(F)(F)F)cc1=O)c1cc(ccc1F)O2]

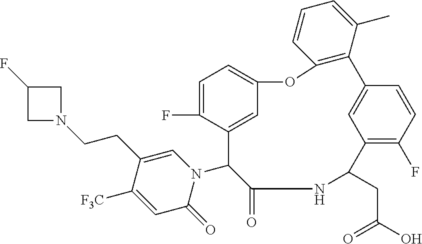 [SMILES: Cc1cccc2c1-c1ccc(F)c(c1)C(CC(=O)O)NC(=O)C(n1cc(CCN3CC(F)C3)c(C(F)(F)F)cc1=O)c1cc(ccc1F)O2]

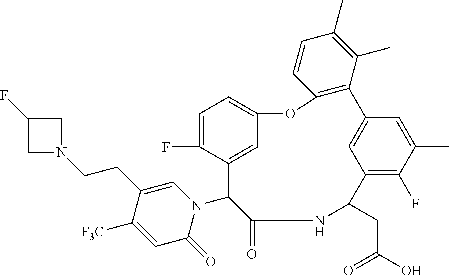 [SMILES: Cc1ccc2c(c1C)-c1cc(C)c(F)c(c1)C(CC(=O)O)NC(=O)C(n1cc(CCN3CC(F)C3)c(C(F)(F)F)cc1=O)c1cc(ccc1F)O2]

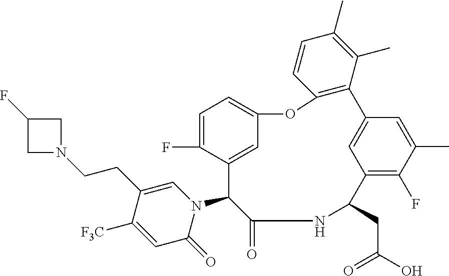 [SMILES: Cc1ccc2c(c1C)-c1cc(C)c(F)c(c1)[C@@H](CC(=O)O)NC(=O)[C@@H](n1cc(CCN3CC(F)C3)c(C(F)(F)F)cc1=O)c1cc(ccc1F)O2]

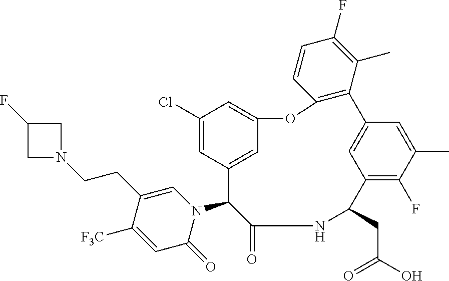 [SMILES: Cc1cc2cc(c1F)[C@@H](CC(=O)O)NC(=O)[C@@H](n1cc(CCN3CC(F)C3)c(C(F)(F)F)cc1=O)c1cc(Cl)cc(c1)Oc1ccc(F)c(C)c1-2]

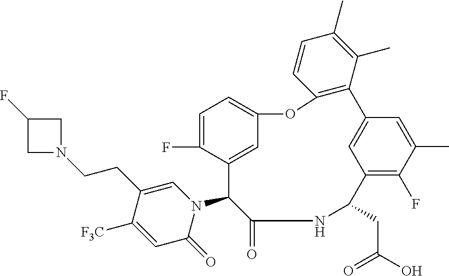 [SMILES: Cc1ccc2c(c1C)-c1cc(C)c(F)c(c1)[C@H](CC(=O)O)NC(=O)[C@@H](n1cc(CCN3CC(F)C3)c(C(F)(F)F)cc1=O)c1cc(ccc1F)O2]

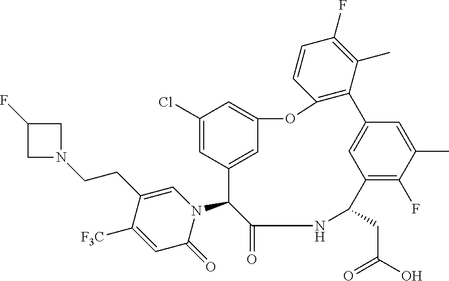 [SMILES: Cc1cc2cc(c1F)[C@H](CC(=O)O)NC(=O)[C@@H](n1cc(CCN3CC(F)C3)c(C(F)(F)F)cc1=O)c1cc(Cl)cc(c1)Oc1ccc(F)c(C)c1-2]

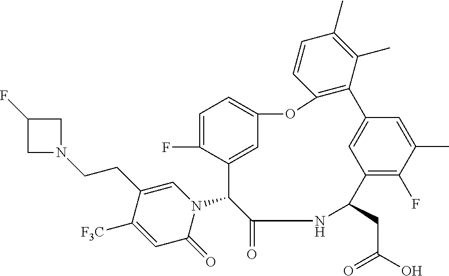 [SMILES: Cc1ccc2c(c1C)-c1cc(C)c(F)c(c1)[C@@H](CC(=O)O)NC(=O)[C@H](n1cc(CCN3CC(F)C3)c(C(F)(F)F)cc1=O)c1cc(ccc1F)O2]

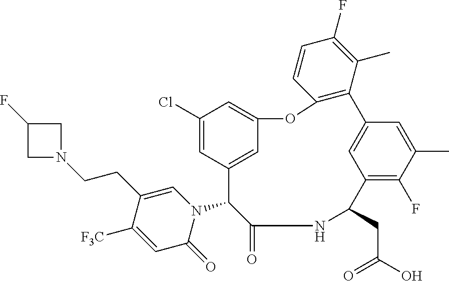 [SMILES: Cc1cc2cc(c1F)[C@@H](CC(=O)O)NC(=O)[C@H](n1cc(CCN3CC(F)C3)c(C(F)(F)F)cc1=O)c1cc(Cl)cc(c1)Oc1ccc(F)c(C)c1-2]

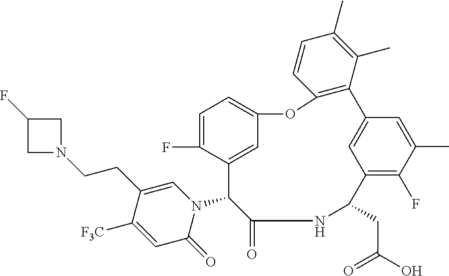 [SMILES: Cc1ccc2c(c1C)-c1cc(C)c(F)c(c1)[C@H](CC(=O)O)NC(=O)[C@H](n1cc(CCN3CC(F)C3)c(C(F)(F)F)cc1=O)c1cc(ccc1F)O2]

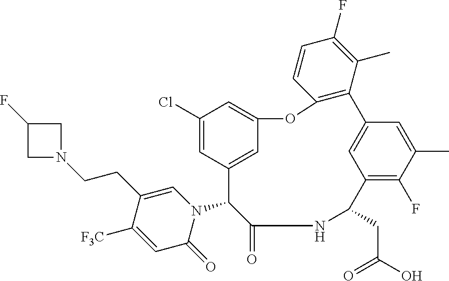 [SMILES: Cc1cc2cc(c1F)[C@H](CC(=O)O)NC(=O)[C@H](n1cc(CCN3CC(F)C3)c(C(F)(F)F)cc1=O)c1cc(Cl)cc(c1)Oc1ccc(F)c(C)c1-2]

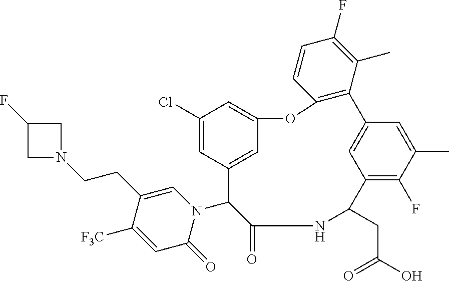 [SMILES: Cc1cc2cc(c1F)C(CC(=O)O)NC(=O)C(n1cc(CCN3CC(F)C3)c(C(F)(F)F)cc1=O)c1cc(Cl)cc(c1)Oc1ccc(F)c(C)c1-2]

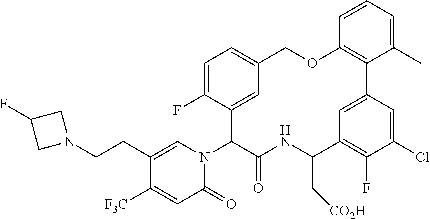 [SMILES: Cc1cccc2c1-c1cc(Cl)c(F)c(c1)C(CC(=O)O)NC(=O)C(n1cc(CCN3CC(F)C3)c(C(F)(F)F)cc1=O)c1cc(ccc1F)CO2]

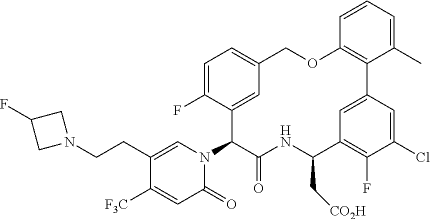 [SMILES: Cc1cccc2c1-c1cc(Cl)c(F)c(c1)[C@@H](CC(=O)O)NC(=O)[C@@H](n1cc(CCN3CC(F)C3)c(C(F)(F)F)cc1=O)c1cc(ccc1F)CO2]

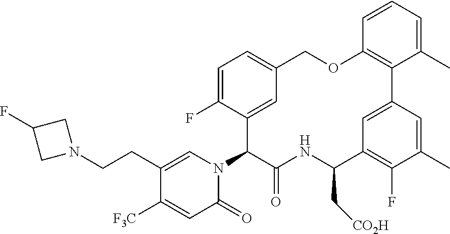 [SMILES: Cc1cc2cc(c1F)[C@@H](CC(=O)O)NC(=O)[C@@H](n1cc(CCN3CC(F)C3)c(C(F)(F)F)cc1=O)c1cc(ccc1F)COc1cccc(C)c1-2]

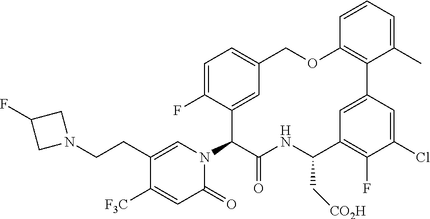 [SMILES: Cc1cccc2c1-c1cc(Cl)c(F)c(c1)[C@H](CC(=O)O)NC(=O)[C@@H](n1cc(CCN3CC(F)C3)c(C(F)(F)F)cc1=O)c1cc(ccc1F)CO2]

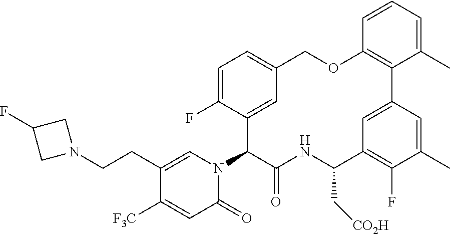 [SMILES: Cc1cc2cc(c1F)[C@H](CC(=O)O)NC(=O)[C@@H](n1cc(CCN3CC(F)C3)c(C(F)(F)F)cc1=O)c1cc(ccc1F)COc1cccc(C)c1-2]

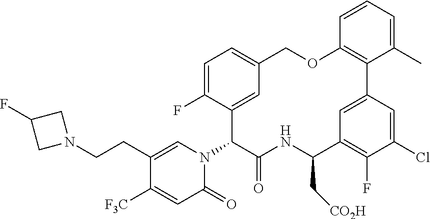 [SMILES: Cc1cccc2c1-c1cc(Cl)c(F)c(c1)[C@@H](CC(=O)O)NC(=O)[C@H](n1cc(CCN3CC(F)C3)c(C(F)(F)F)cc1=O)c1cc(ccc1F)CO2]

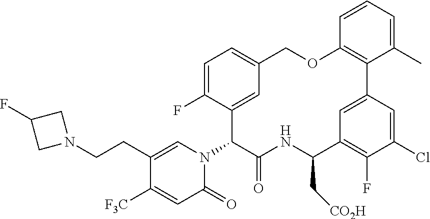 [SMILES: Cc1cccc2c1-c1cc(Cl)c(F)c(c1)[C@@H](CC(=O)O)NC(=O)[C@H](n1cc(CCN3CC(F)C3)c(C(F)(F)F)cc1=O)c1cc(ccc1F)CO2]

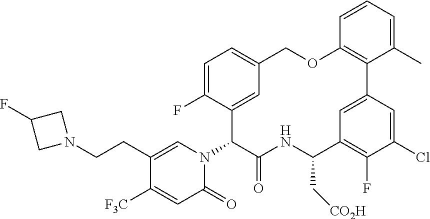 [SMILES: Cc1cccc2c1-c1cc(Cl)c(F)c(c1)[C@H](CC(=O)O)NC(=O)[C@H](n1cc(CCN3CC(F)C3)c(C(F)(F)F)cc1=O)c1cc(ccc1F)CO2]

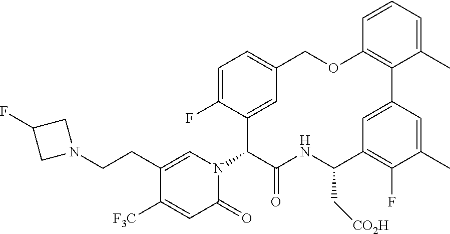 [SMILES: Cc1cc2cc(c1F)[C@H](CC(=O)O)NC(=O)[C@H](n1cc(CCN3CC(F)C3)c(C(F)(F)F)cc1=O)c1cc(ccc1F)COc1cccc(C)c1-2]

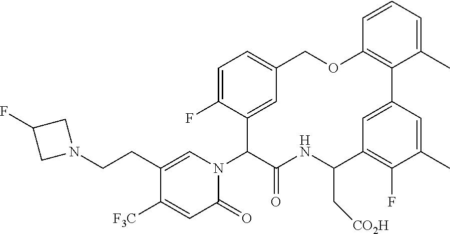 [SMILES: Cc1cc2cc(c1F)C(CC(=O)O)NC(=O)C(n1cc(CCN3CC(F)C3)c(C(F)(F)F)cc1=O)c1cc(ccc1F)COc1cccc(C)c1-2]

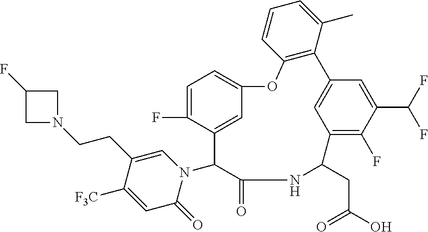 [SMILES: Cc1cccc2c1-c1cc(C(F)F)c(F)c(c1)C(CC(=O)O)NC(=O)C(n1cc(CCN3CC(F)C3)c(C(F)(F)F)cc1=O)c1cc(ccc1F)O2]

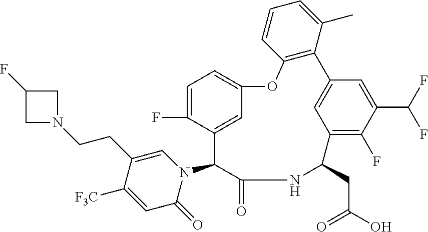 [SMILES: Cc1cccc2c1-c1cc(C(F)F)c(F)c(c1)[C@@H](CC(=O)O)NC(=O)[C@@H](n1cc(CCN3CC(F)C3)c(C(F)(F)F)cc1=O)c1cc(ccc1F)O2]

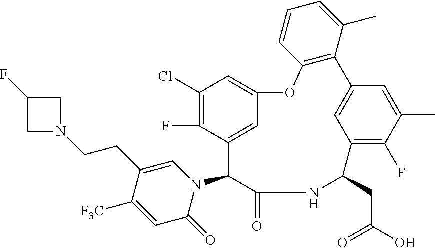 [SMILES: Cc1cc2cc(c1F)[C@@H](CC(=O)O)NC(=O)[C@@H](n1cc(CCN3CC(F)C3)c(C(F)(F)F)cc1=O)c1cc(cc(Cl)c1F)Oc1cccc(C)c1-2]

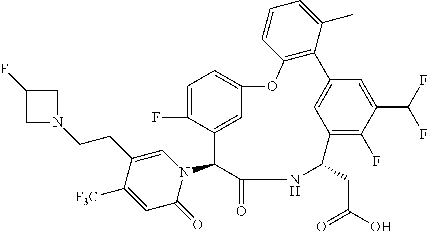 [SMILES: Cc1cccc2c1-c1cc(C(F)F)c(F)c(c1)[C@H](CC(=O)O)NC(=O)[C@@H](n1cc(CCN3CC(F)C3)c(C(F)(F)F)cc1=O)c1cc(ccc1F)O2]

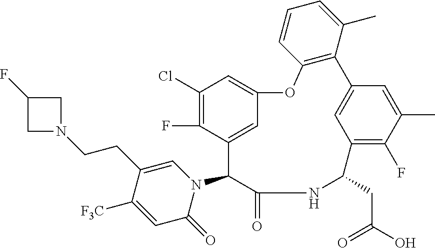 [SMILES: Cc1cc2cc(c1F)[C@H](CC(=O)O)NC(=O)[C@@H](n1cc(CCN3CC(F)C3)c(C(F)(F)F)cc1=O)c1cc(cc(Cl)c1F)Oc1cccc(C)c1-2]

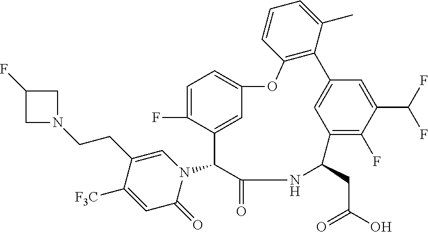 [SMILES: Cc1cccc2c1-c1cc(C(F)F)c(F)c(c1)[C@@H](CC(=O)O)NC(=O)[C@H](n1cc(CCN3CC(F)C3)c(C(F)(F)F)cc1=O)c1cc(ccc1F)O2]

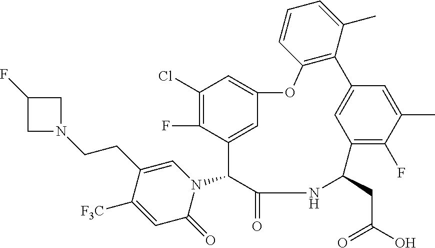 [SMILES: Cc1cc2cc(c1F)[C@@H](CC(=O)O)NC(=O)[C@H](n1cc(CCN3CC(F)C3)c(C(F)(F)F)cc1=O)c1cc(cc(Cl)c1F)Oc1cccc(C)c1-2]

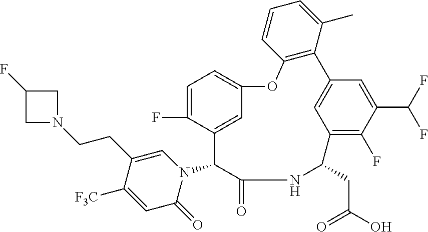 [SMILES: Cc1cccc2c1-c1cc(C(F)F)c(F)c(c1)[C@H](CC(=O)O)NC(=O)[C@H](n1cc(CCN3CC(F)C3)c(C(F)(F)F)cc1=O)c1cc(ccc1F)O2]

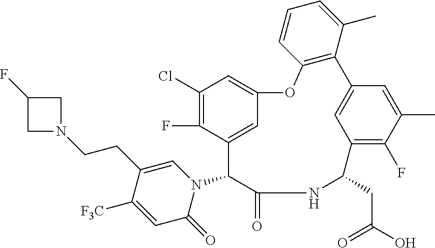 [SMILES: Cc1cc2cc(c1F)[C@H](CC(=O)O)NC(=O)[C@H](n1cc(CCN3CC(F)C3)c(C(F)(F)F)cc1=O)c1cc(cc(Cl)c1F)Oc1cccc(C)c1-2]

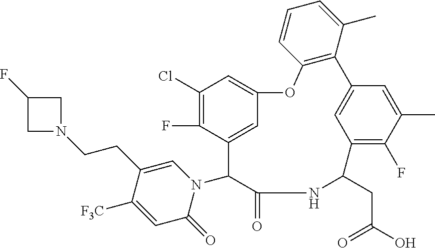 [SMILES: Cc1cc2cc(c1F)C(CC(=O)O)NC(=O)C(n1cc(CCN3CC(F)C3)c(C(F)(F)F)cc1=O)c1cc(cc(Cl)c1F)Oc1cccc(C)c1-2]

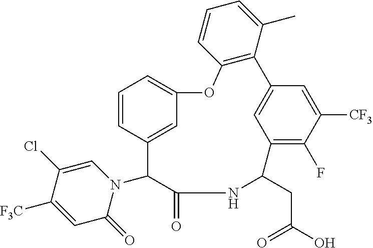 [SMILES: Cc1cccc2c1-c1cc(c(F)c(C(F)(F)F)c1)C(CC(=O)O)NC(=O)C(n1cc(Cl)c(C(F)(F)F)cc1=O)c1cccc(c1)O2]

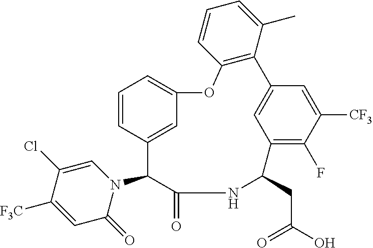 [SMILES: Cc1cccc2c1-c1cc(c(F)c(C(F)(F)F)c1)[C@@H](CC(=O)O)NC(=O)[C@@H](n1cc(Cl)c(C(F)(F)F)cc1=O)c1cccc(c1)O2]

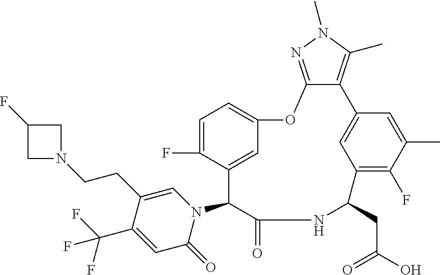 [SMILES: Cc1cc2cc(c1F)[C@@H](CC(=O)O)NC(=O)[C@@H](n1cc(CCN3CC(F)C3)c(C(F)(F)F)cc1=O)c1cc(ccc1F)Oc1nn(C)c(C)c1-2]

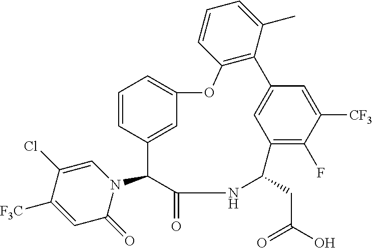 [SMILES: Cc1cccc2c1-c1cc(c(F)c(C(F)(F)F)c1)[C@H](CC(=O)O)NC(=O)[C@@H](n1cc(Cl)c(C(F)(F)F)cc1=O)c1cccc(c1)O2]

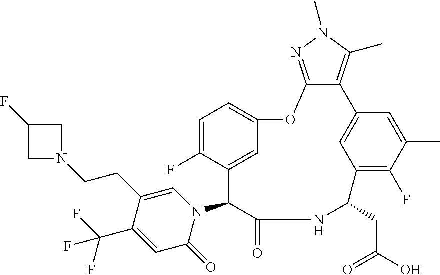 [SMILES: Cc1cc2cc(c1F)[C@H](CC(=O)O)NC(=O)[C@@H](n1cc(CCN3CC(F)C3)c(C(F)(F)F)cc1=O)c1cc(ccc1F)Oc1nn(C)c(C)c1-2]

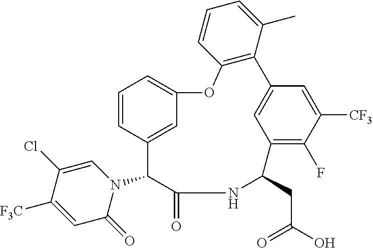 [SMILES: Cc1cccc2c1-c1cc(c(F)c(C(F)(F)F)c1)[C@@H](CC(=O)O)NC(=O)[C@H](n1cc(Cl)c(C(F)(F)F)cc1=O)c1cccc(c1)O2]

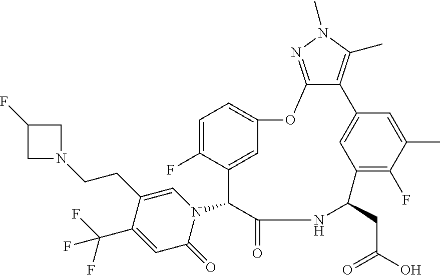 [SMILES: Cc1cc2cc(c1F)[C@@H](CC(=O)O)NC(=O)[C@H](n1cc(CCN3CC(F)C3)c(C(F)(F)F)cc1=O)c1cc(ccc1F)Oc1nn(C)c(C)c1-2]

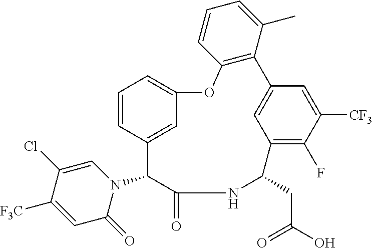 [SMILES: Cc1cccc2c1-c1cc(c(F)c(C(F)(F)F)c1)[C@H](CC(=O)O)NC(=O)[C@H](n1cc(Cl)c(C(F)(F)F)cc1=O)c1cccc(c1)O2]

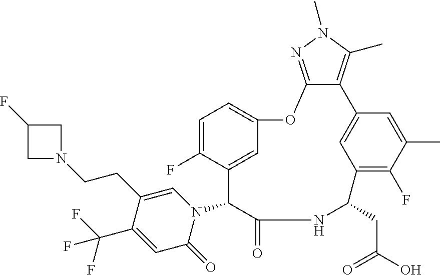 [SMILES: Cc1cc2cc(c1F)[C@H](CC(=O)O)NC(=O)[C@H](n1cc(CCN3CC(F)C3)c(C(F)(F)F)cc1=O)c1cc(ccc1F)Oc1nn(C)c(C)c1-2]

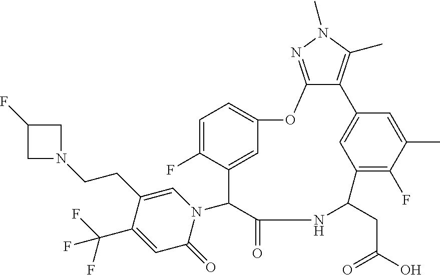 [SMILES: Cc1cc2cc(c1F)C(CC(=O)O)NC(=O)C(n1cc(CCN3CC(F)C3)c(C(F)(F)F)cc1=O)c1cc(ccc1F)Oc1nn(C)c(C)c1-2]

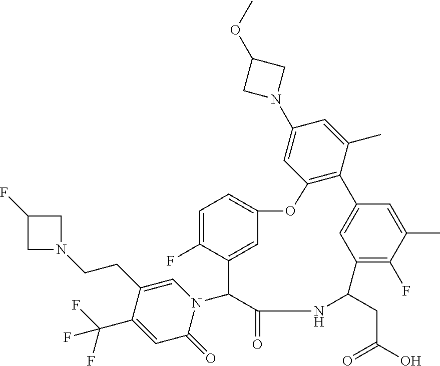 [SMILES: COC1CN(c2cc(C)c3c(c2)Oc2ccc(F)c(c2)C(n2cc(CCN4CC(F)C4)c(C(F)(F)F)cc2=O)C(=O)NC(CC(=O)O)c2cc-3cc(C)c2F)C1]

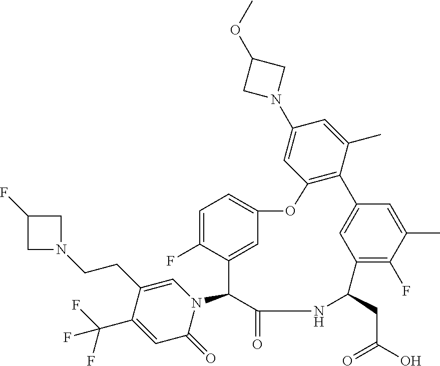 [SMILES: COC1CN(c2cc(C)c3c(c2)Oc2ccc(F)c(c2)[C@H](n2cc(CCN4CC(F)C4)c(C(F)(F)F)cc2=O)C(=O)N[C@H](CC(=O)O)c2cc-3cc(C)c2F)C1]

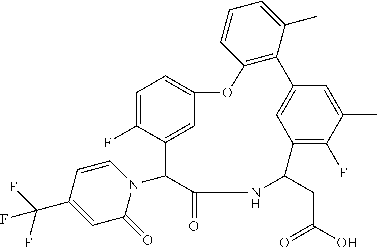 [SMILES: Cc1cc2cc(c1F)C(CC(=O)O)NC(=O)C(n1ccc(C(F)(F)F)cc1=O)c1cc(ccc1F)Oc1cccc(C)c1-2]

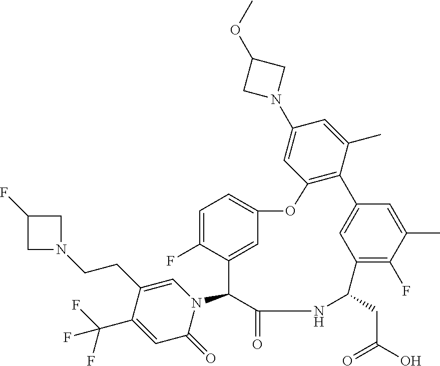 [SMILES: COC1CN(c2cc(C)c3c(c2)Oc2ccc(F)c(c2)[C@H](n2cc(CCN4CC(F)C4)c(C(F)(F)F)cc2=O)C(=O)N[C@@H](CC(=O)O)c2cc-3cc(C)c2F)C1]

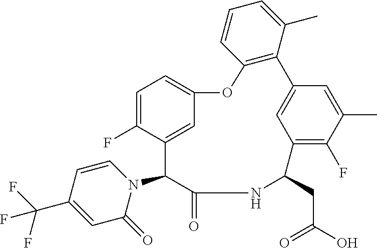 [SMILES: Cc1cc2cc(c1F)[C@@H](CC(=O)O)NC(=O)[C@@H](n1ccc(C(F)(F)F)cc1=O)c1cc(ccc1F)Oc1cccc(C)c1-2]

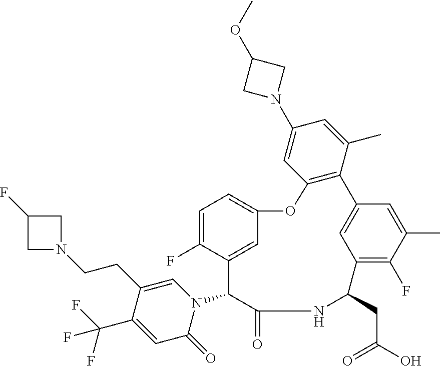 [SMILES: COC1CN(c2cc(C)c3c(c2)Oc2ccc(F)c(c2)[C@@H](n2cc(CCN4CC(F)C4)c(C(F)(F)F)cc2=O)C(=O)N[C@H](CC(=O)O)c2cc-3cc(C)c2F)C1]

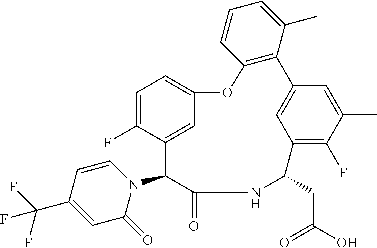 [SMILES: Cc1cc2cc(c1F)[C@H](CC(=O)O)NC(=O)[C@@H](n1ccc(C(F)(F)F)cc1=O)c1cc(ccc1F)Oc1cccc(C)c1-2]

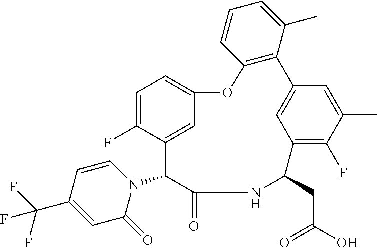 [SMILES: Cc1cc2cc(c1F)[C@@H](CC(=O)O)NC(=O)[C@H](n1ccc(C(F)(F)F)cc1=O)c1cc(ccc1F)Oc1cccc(C)c1-2]

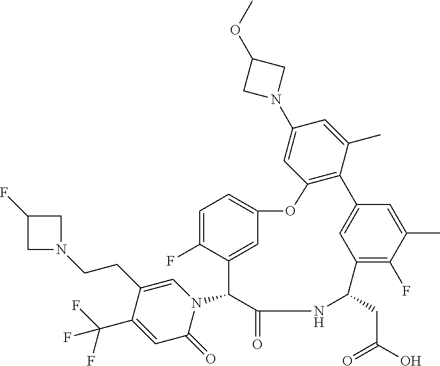 [SMILES: COC1CN(c2cc(C)c3c(c2)Oc2ccc(F)c(c2)[C@@H](n2cc(CCN4CC(F)C4)c(C(F)(F)F)cc2=O)C(=O)N[C@@H](CC(=O)O)c2cc-3cc(C)c2F)C1]

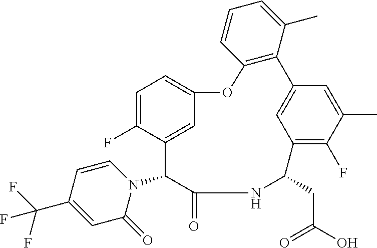 [SMILES: Cc1cc2cc(c1F)[C@H](CC(=O)O)NC(=O)[C@H](n1ccc(C(F)(F)F)cc1=O)c1cc(ccc1F)Oc1cccc(C)c1-2]

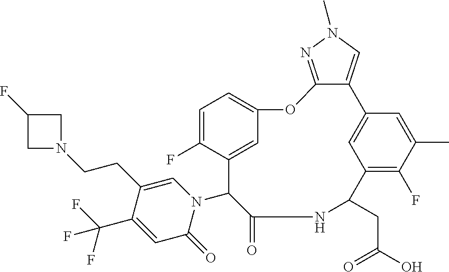 [SMILES: Cc1cc2cc(c1F)C(CC(=O)O)NC(=O)C(n1cc(CCN3CC(F)C3)c(C(F)(F)F)cc1=O)c1cc(ccc1F)Oc1nn(C)cc1-2]

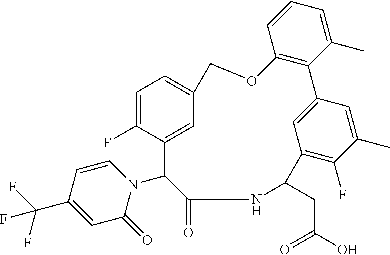 [SMILES: Cc1cc2cc(c1F)C(CC(=O)O)NC(=O)C(n1ccc(C(F)(F)F)cc1=O)c1cc(ccc1F)COc1cccc(C)c1-2]

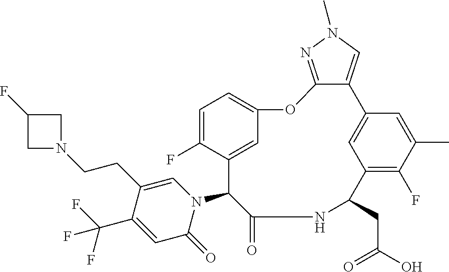 [SMILES: Cc1cc2cc(c1F)[C@@H](CC(=O)O)NC(=O)[C@@H](n1cc(CCN3CC(F)C3)c(C(F)(F)F)cc1=O)c1cc(ccc1F)Oc1nn(C)cc1-2]

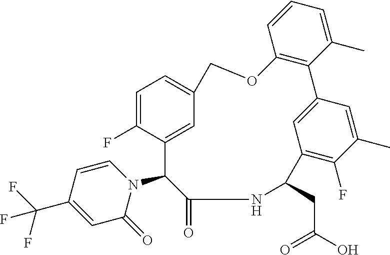 [SMILES: Cc1cc2cc(c1F)[C@@H](CC(=O)O)NC(=O)[C@@H](n1ccc(C(F)(F)F)cc1=O)c1cc(ccc1F)COc1cccc(C)c1-2]

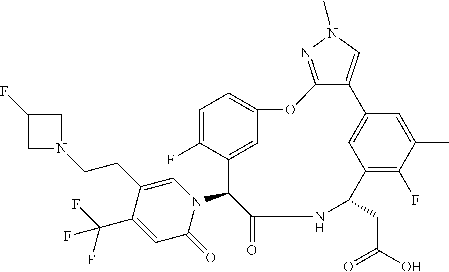 [SMILES: Cc1cc2cc(c1F)[C@H](CC(=O)O)NC(=O)[C@@H](n1cc(CCN3CC(F)C3)c(C(F)(F)F)cc1=O)c1cc(ccc1F)Oc1nn(C)cc1-2]

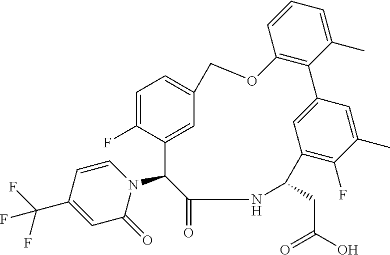 [SMILES: Cc1cc2cc(c1F)[C@H](CC(=O)O)NC(=O)[C@@H](n1ccc(C(F)(F)F)cc1=O)c1cc(ccc1F)COc1cccc(C)c1-2]

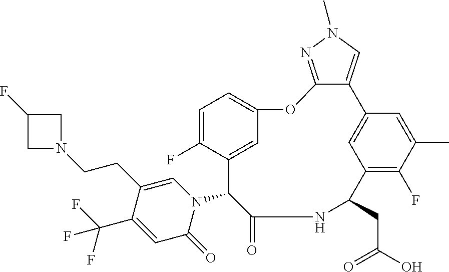 [SMILES: Cc1cc2cc(c1F)[C@@H](CC(=O)O)NC(=O)[C@H](n1cc(CCN3CC(F)C3)c(C(F)(F)F)cc1=O)c1cc(ccc1F)Oc1nn(C)cc1-2]

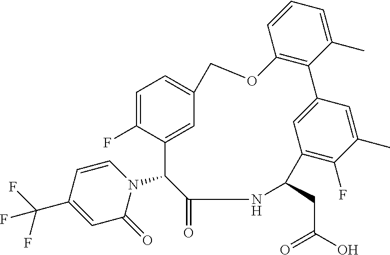 [SMILES: Cc1cc2cc(c1F)[C@@H](CC(=O)O)NC(=O)[C@H](n1ccc(C(F)(F)F)cc1=O)c1cc(ccc1F)COc1cccc(C)c1-2]

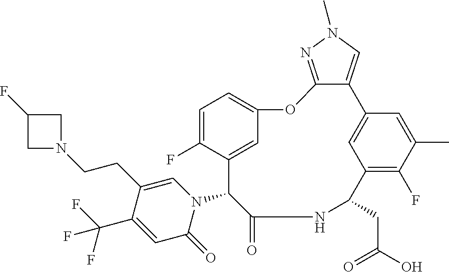 [SMILES: Cc1cc2cc(c1F)[C@H](CC(=O)O)NC(=O)[C@H](n1cc(CCN3CC(F)C3)c(C(F)(F)F)cc1=O)c1cc(ccc1F)Oc1nn(C)cc1-2]

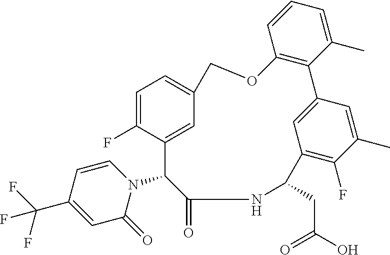 [SMILES: Cc1cc2cc(c1F)[C@H](CC(=O)O)NC(=O)[C@H](n1ccc(C(F)(F)F)cc1=O)c1cc(ccc1F)COc1cccc(C)c1-2]

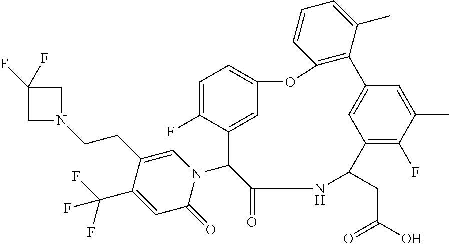 [SMILES: Cc1cc2cc(c1F)C(CC(=O)O)NC(=O)C(n1cc(CCN3CC(F)(F)C3)c(C(F)(F)F)cc1=O)c1cc(ccc1F)Oc1cccc(C)c1-2]

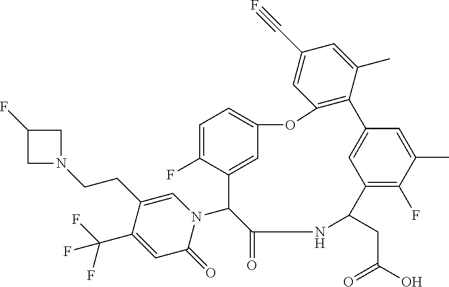

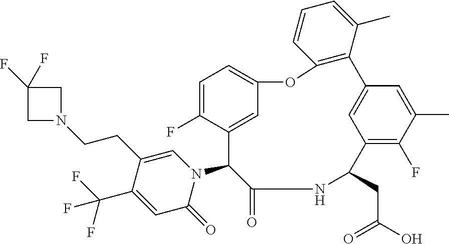 [SMILES: Cc1cc2cc(c1F)[C@@H](CC(=O)O)NC(=O)[C@@H](n1cc(CCN3CC(F)(F)C3)c(C(F)(F)F)cc1=O)c1cc(ccc1F)Oc1cccc(C)c1-2]

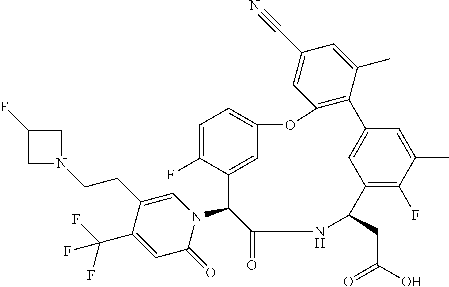 [SMILES: Cc1cc2cc(c1F)[C@@H](CC(=O)O)NC(=O)[C@@H](n1cc(CCN3CC(F)C3)c(C(F)(F)F)cc1=O)c1cc(ccc1F)Oc1cc(C#N)cc(C)c1-2]

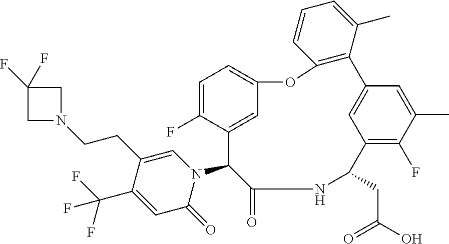 [SMILES: Cc1cc2cc(c1F)[C@H](CC(=O)O)NC(=O)[C@@H](n1cc(CCN3CC(F)(F)C3)c(C(F)(F)F)cc1=O)c1cc(ccc1F)Oc1cccc(C)c1-2]

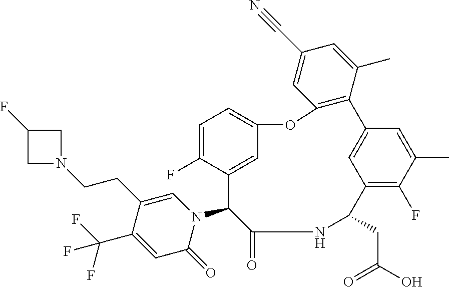 [SMILES: Cc1cc2cc(c1F)[C@H](CC(=O)O)NC(=O)[C@@H](n1cc(CCN3CC(F)C3)c(C(F)(F)F)cc1=O)c1cc(ccc1F)Oc1cc(C#N)cc(C)c1-2]

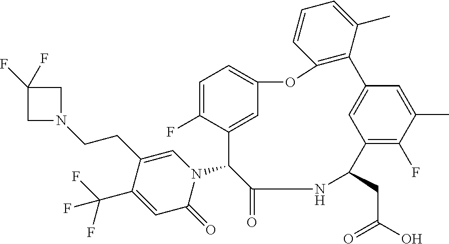 [SMILES: Cc1cc2cc(c1F)[C@@H](CC(=O)O)NC(=O)[C@H](n1cc(CCN3CC(F)(F)C3)c(C(F)(F)F)cc1=O)c1cc(ccc1F)Oc1cccc(C)c1-2]

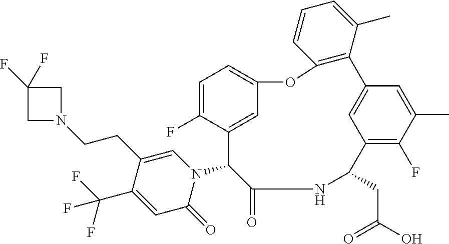 [SMILES: Cc1cc2cc(c1F)[C@H](CC(=O)O)NC(=O)[C@H](n1cc(CCN3CC(F)(F)C3)c(C(F)(F)F)cc1=O)c1cc(ccc1F)Oc1cccc(C)c1-2]

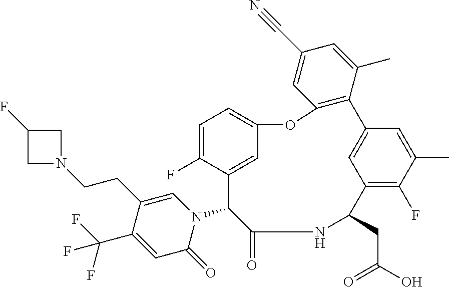 [SMILES: Cc1cc2cc(c1F)[C@@H](CC(=O)O)NC(=O)[C@H](n1cc(CCN3CC(F)C3)c(C(F)(F)F)cc1=O)c1cc(ccc1F)Oc1cc(C#N)cc(C)c1-2]

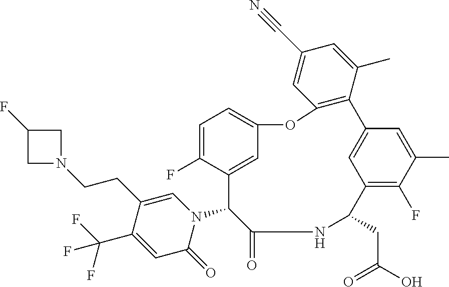 [SMILES: Cc1cc2cc(c1F)[C@H](CC(=O)O)NC(=O)[C@H](n1cc(CCN3CC(F)C3)c(C(F)(F)F)cc1=O)c1cc(ccc1F)Oc1cc(C#N)cc(C)c1-2]

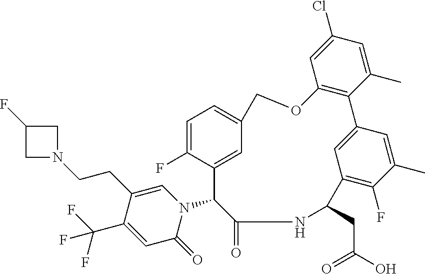 [SMILES: Cc1cc2cc(c1F)[C@@H](CC(=O)O)NC(=O)[C@H](n1cc(CCN3CC(F)C3)c(C(F)(F)F)cc1=O)c1cc(ccc1F)COc1cc(Cl)cc(C)c1-2]

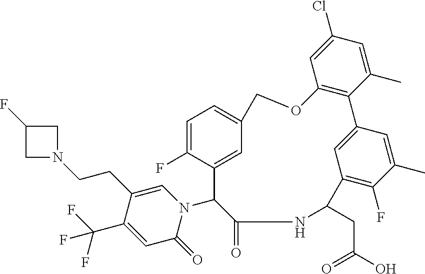 [SMILES: Cc1cc2cc(c1F)C(CC(=O)O)NC(=O)C(n1cc(CCN3CC(F)C3)c(C(F)(F)F)cc1=O)c1cc(ccc1F)COc1cc(Cl)cc(C)c1-2]

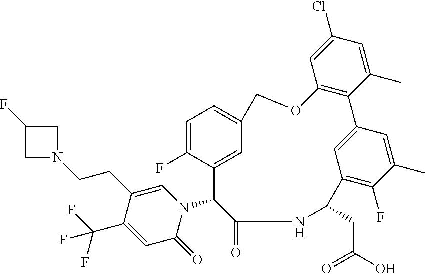 [SMILES: Cc1cc2cc(c1F)[C@H](CC(=O)O)NC(=O)[C@H](n1cc(CCN3CC(F)C3)c(C(F)(F)F)cc1=O)c1cc(ccc1F)COc1cc(Cl)cc(C)c1-2]

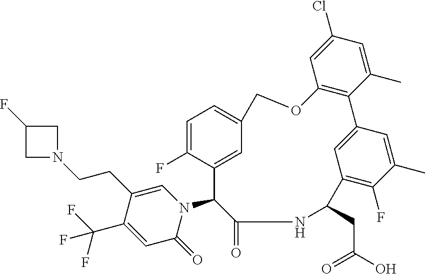 [SMILES: Cc1cc2cc(c1F)[C@@H](CC(=O)O)NC(=O)[C@@H](n1cc(CCN3CC(F)C3)c(C(F)(F)F)cc1=O)c1cc(ccc1F)COc1cc(Cl)cc(C)c1-2]

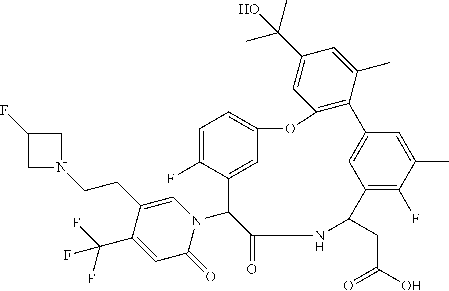 [SMILES: Cc1cc2cc(c1F)C(CC(=O)O)NC(=O)C(n1cc(CCN3CC(F)C3)c(C(F)(F)F)cc1=O)c1cc(ccc1F)Oc1cc(C(C)(C)O)cc(C)c1-2]

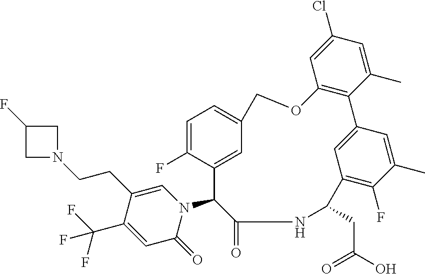 [SMILES: Cc1cc2cc(c1F)[C@H](CC(=O)O)NC(=O)[C@@H](n1cc(CCN3CC(F)C3)c(C(F)(F)F)cc1=O)c1cc(ccc1F)COc1cc(Cl)cc(C)c1-2]

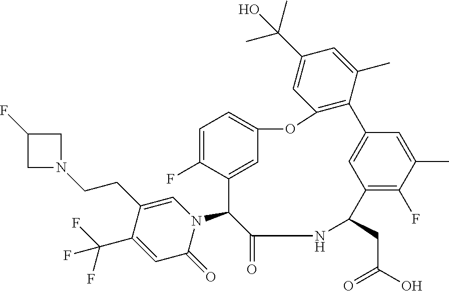 [SMILES: Cc1cc2cc(c1F)[C@@H](CC(=O)O)NC(=O)[C@@H](n1cc(CCN3CC(F)C3)c(C(F)(F)F)cc1=O)c1cc(ccc1F)Oc1cc(C(C)(C)O)cc(C)c1-2]

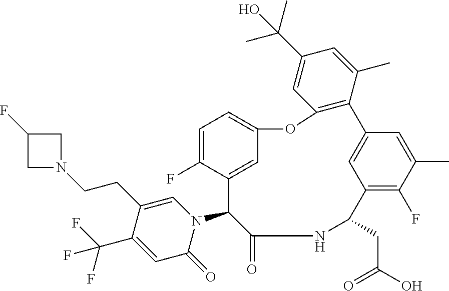 [SMILES: Cc1cc2cc(c1F)[C@H](CC(=O)O)NC(=O)[C@@H](n1cc(CCN3CC(F)C3)c(C(F)(F)F)cc1=O)c1cc(ccc1F)Oc1cc(C(C)(C)O)cc(C)c1-2]

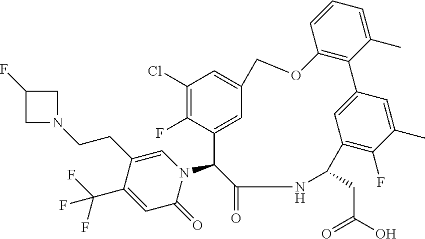 [SMILES: Cc1cc2cc(c1F)[C@H](CC(=O)O)NC(=O)[C@@H](n1cc(CCN3CC(F)C3)c(C(F)(F)F)cc1=O)c1cc(cc(Cl)c1F)COc1cccc(C)c1-2]

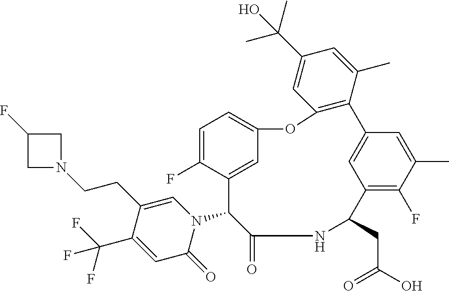 [SMILES: Cc1cc2cc(c1F)[C@@H](CC(=O)O)NC(=O)[C@H](n1cc(CCN3CC(F)C3)c(C(F)(F)F)cc1=O)c1cc(ccc1F)Oc1cc(C(C)(C)O)cc(C)c1-2]

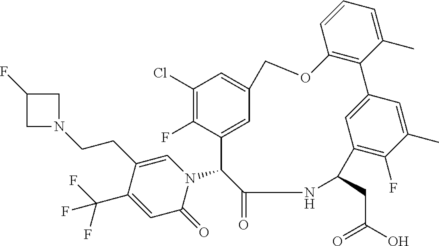 [SMILES: Cc1cc2cc(c1F)[C@@H](CC(=O)O)NC(=O)[C@H](n1cc(CCN3CC(F)C3)c(C(F)(F)F)cc1=O)c1cc(cc(Cl)c1F)COc1cccc(C)c1-2]

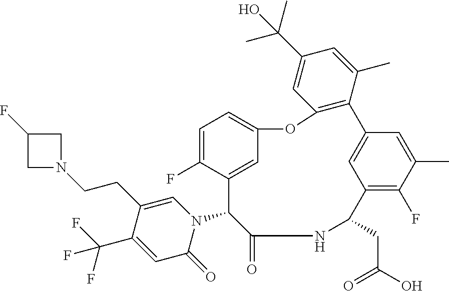 [SMILES: Cc1cc2cc(c1F)[C@H](CC(=O)O)NC(=O)[C@H](n1cc(CCN3CC(F)C3)c(C(F)(F)F)cc1=O)c1cc(ccc1F)Oc1cc(C(C)(C)O)cc(C)c1-2]

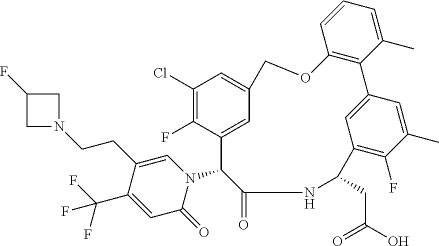 [SMILES: Cc1cc2cc(c1F)[C@H](CC(=O)O)NC(=O)[C@H](n1cc(CCN3CC(F)C3)c(C(F)(F)F)cc1=O)c1cc(cc(Cl)c1F)COc1cccc(C)c1-2]

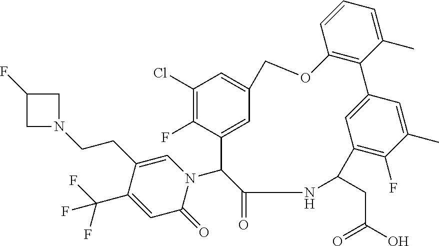 [SMILES: Cc1cc2cc(c1F)C(CC(=O)O)NC(=O)C(n1cc(CCN3CC(F)C3)c(C(F)(F)F)cc1=O)c1cc(cc(Cl)c1F)COc1cccc(C)c1-2]

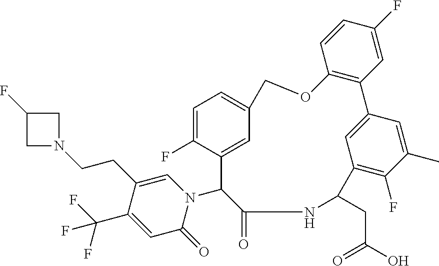 [SMILES: Cc1cc2cc(c1F)C(CC(=O)O)NC(=O)C(n1cc(CCN3CC(F)C3)c(C(F)(F)F)cc1=O)c1cc(ccc1F)COc1ccc(F)cc1-2]

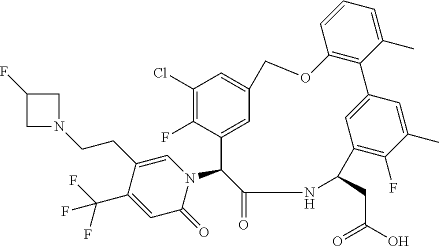 [SMILES: Cc1cc2cc(c1F)[C@@H](CC(=O)O)NC(=O)[C@@H](n1cc(CCN3CC(F)C3)c(C(F)(F)F)cc1=O)c1cc(cc(Cl)c1F)COc1cccc(C)c1-2]

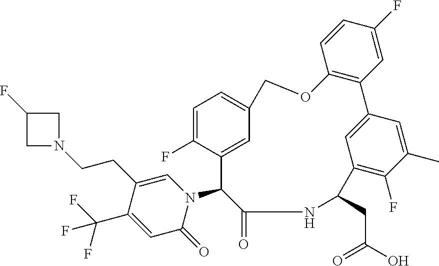 [SMILES: Cc1cc2cc(c1F)[C@@H](CC(=O)O)NC(=O)[C@@H](n1cc(CCN3CC(F)C3)c(C(F)(F)F)cc1=O)c1cc(ccc1F)COc1ccc(F)cc1-2]

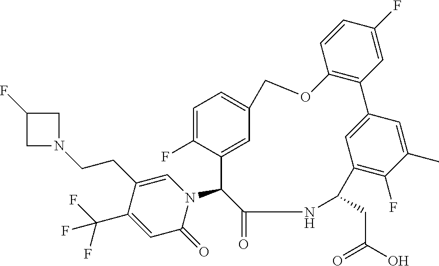 [SMILES: Cc1cc2cc(c1F)[C@H](CC(=O)O)NC(=O)[C@@H](n1cc(CCN3CC(F)C3)c(C(F)(F)F)cc1=O)c1cc(ccc1F)COc1ccc(F)cc1-2]

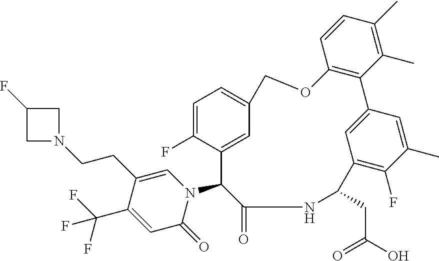 [SMILES: Cc1ccc2c(c1C)-c1cc(C)c(F)c(c1)[C@H](CC(=O)O)NC(=O)[C@@H](n1cc(CCN3CC(F)C3)c(C(F)(F)F)cc1=O)c1cc(ccc1F)CO2]

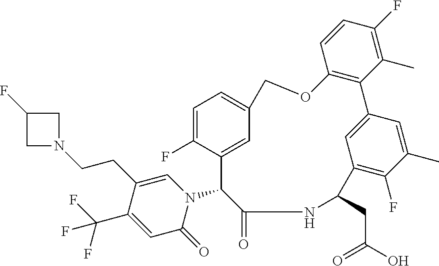 [SMILES: Cc1cc2cc(c1F)[C@@H](CC(=O)O)NC(=O)[C@H](n1cc(CCN3CC(F)C3)c(C(F)(F)F)cc1=O)c1cc(ccc1F)COc1ccc(F)c(C)c1-2]

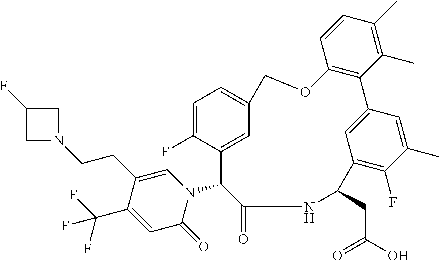 [SMILES: Cc1ccc2c(c1C)-c1cc(C)c(F)c(c1)[C@@H](CC(=O)O)NC(=O)[C@H](n1cc(CCN3CC(F)C3)c(C(F)(F)F)cc1=O)c1cc(ccc1F)CO2]

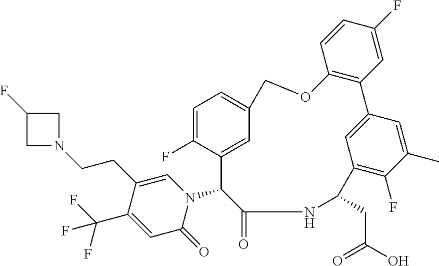 [SMILES: Cc1cc2cc(c1F)[C@H](CC(=O)O)NC(=O)[C@H](n1cc(CCN3CC(F)C3)c(C(F)(F)F)cc1=O)c1cc(ccc1F)COc1ccc(F)cc1-2]

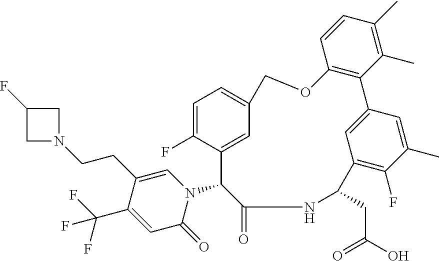 [SMILES: Cc1ccc2c(c1C)-c1cc(C)c(F)c(c1)[C@H](CC(=O)O)NC(=O)[C@H](n1cc(CCN3CC(F)C3)c(C(F)(F)F)cc1=O)c1cc(ccc1F)CO2]

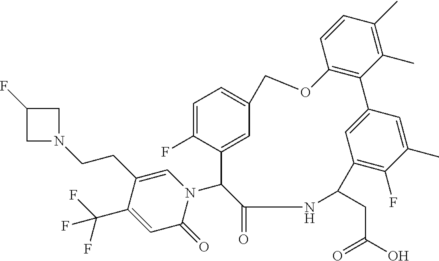 [SMILES: Cc1ccc2c(c1C)-c1cc(C)c(F)c(c1)C(CC(=O)O)NC(=O)C(n1cc(CCN3CC(F)C3)c(C(F)(F)F)cc1=O)c1cc(ccc1F)CO2]

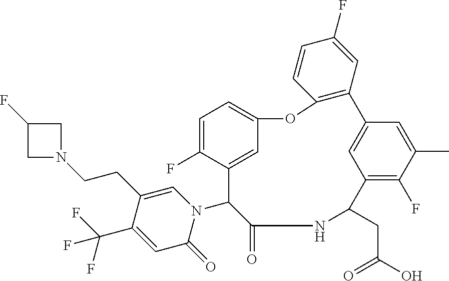 [SMILES: Cc1cc2cc(c1F)C(CC(=O)O)NC(=O)C(n1cc(CCN3CC(F)C3)c(C(F)(F)F)cc1=O)c1cc(ccc1F)Oc1ccc(F)cc1-2]

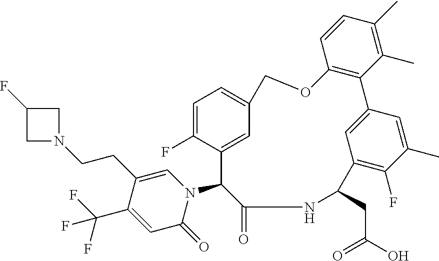 [SMILES: Cc1ccc2c(c1C)-c1cc(C)c(F)c(c1)[C@@H](CC(=O)O)NC(=O)[C@@H](n1cc(CCN3CC(F)C3)c(C(F)(F)F)cc1=O)c1cc(ccc1F)CO2]

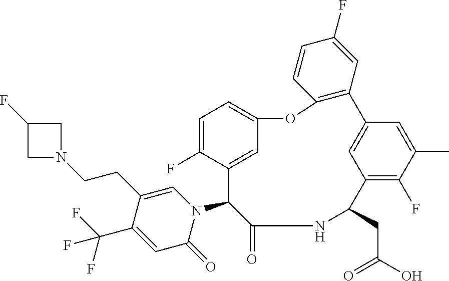 [SMILES: Cc1cc2cc(c1F)[C@@H](CC(=O)O)NC(=O)[C@@H](n1cc(CCN3CC(F)C3)c(C(F)(F)F)cc1=O)c1cc(ccc1F)Oc1ccc(F)cc1-2]

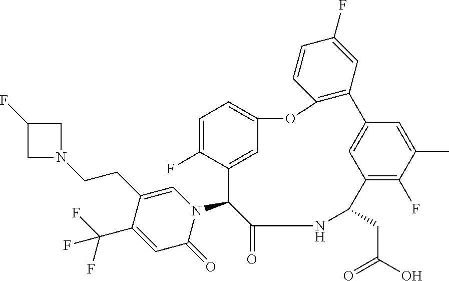 [SMILES: Cc1cc2cc(c1F)[C@H](CC(=O)O)NC(=O)[C@@H](n1cc(CCN3CC(F)C3)c(C(F)(F)F)cc1=O)c1cc(ccc1F)Oc1ccc(F)cc1-2]

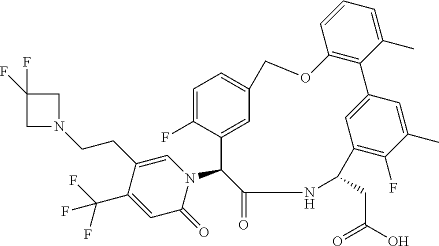 [SMILES: Cc1cc2cc(c1F)[C@H](CC(=O)O)NC(=O)[C@@H](n1cc(CCN3CC(F)(F)C3)c(C(F)(F)F)cc1=O)c1cc(ccc1F)COc1cccc(C)c1-2]

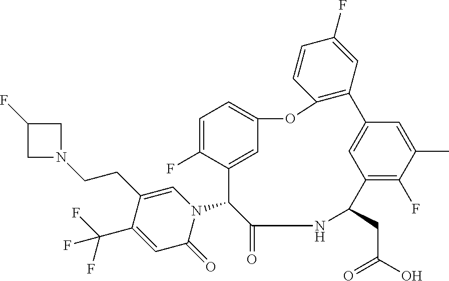 [SMILES: Cc1cc2cc(c1F)[C@@H](CC(=O)O)NC(=O)[C@H](n1cc(CCN3CC(F)C3)c(C(F)(F)F)cc1=O)c1cc(ccc1F)Oc1ccc(F)cc1-2]

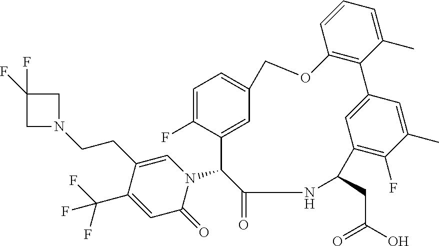 [SMILES: Cc1cc2cc(c1F)[C@@H](CC(=O)O)NC(=O)[C@H](n1cc(CCN3CC(F)(F)C3)c(C(F)(F)F)cc1=O)c1cc(ccc1F)COc1cccc(C)c1-2]

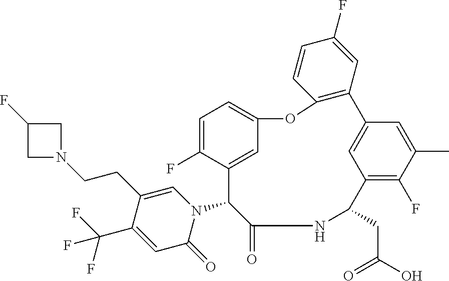 [SMILES: Cc1cc2cc(c1F)[C@H](CC(=O)O)NC(=O)[C@H](n1cc(CCN3CC(F)C3)c(C(F)(F)F)cc1=O)c1cc(ccc1F)Oc1ccc(F)cc1-2]

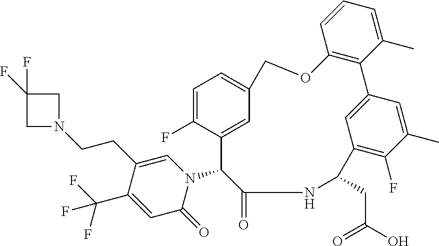 [SMILES: Cc1cc2cc(c1F)[C@H](CC(=O)O)NC(=O)[C@H](n1cc(CCN3CC(F)(F)C3)c(C(F)(F)F)cc1=O)c1cc(ccc1F)COc1cccc(C)c1-2]

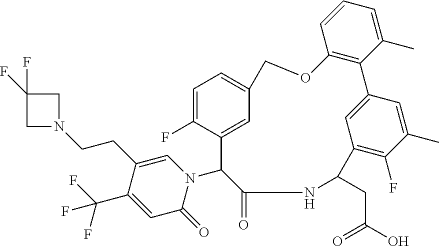 [SMILES: Cc1cc2cc(c1F)C(CC(=O)O)NC(=O)C(n1cc(CCN3CC(F)(F)C3)c(C(F)(F)F)cc1=O)c1cc(ccc1F)COc1cccc(C)c1-2]

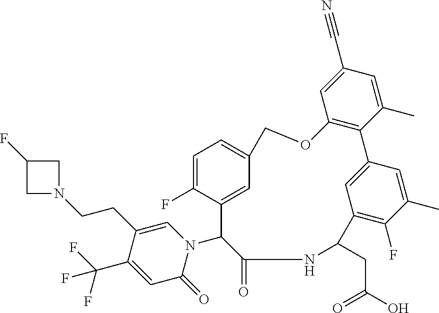 [SMILES: Cc1cc2cc(c1F)C(CC(=O)O)NC(=O)C(n1cc(CCN3CC(F)C3)c(C(F)(F)F)cc1=O)c1cc(ccc1F)COc1cc(C#N)cc(C)c1-2]

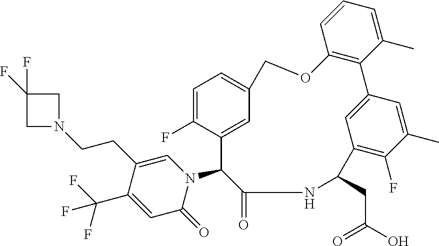 [SMILES: Cc1cc2cc(c1F)[C@@H](CC(=O)O)NC(=O)[C@@H](n1cc(CCN3CC(F)(F)C3)c(C(F)(F)F)cc1=O)c1cc(ccc1F)COc1cccc(C)c1-2]

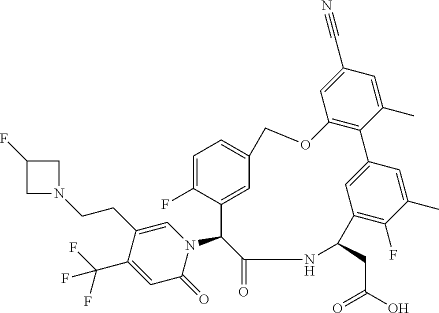 [SMILES: Cc1cc2cc(c1F)[C@@H](CC(=O)O)NC(=O)[C@@H](n1cc(CCN3CC(F)C3)c(C(F)(F)F)cc1=O)c1cc(ccc1F)COc1cc(C#N)cc(C)c1-2]

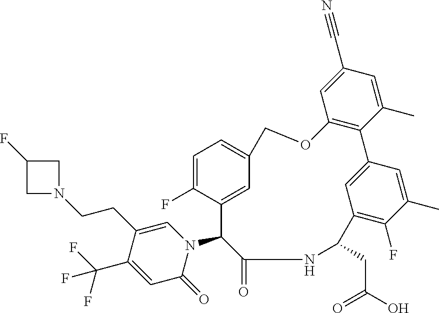 [SMILES: Cc1cc2cc(c1F)[C@H](CC(=O)O)NC(=O)[C@@H](n1cc(CCN3CC(F)C3)c(C(F)(F)F)cc1=O)c1cc(ccc1F)COc1cc(C#N)cc(C)c1-2]

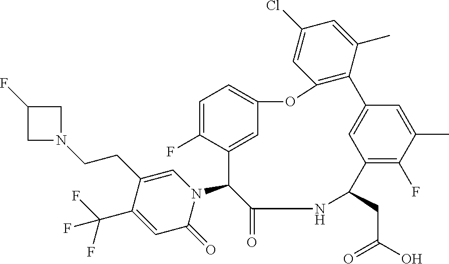 [SMILES: Cc1cc2cc(c1F)[C@@H](CC(=O)O)NC(=O)[C@@H](n1cc(CCN3CC(F)C3)c(C(F)(F)F)cc1=O)c1cc(ccc1F)Oc1cc(Cl)cc(C)c1-2]

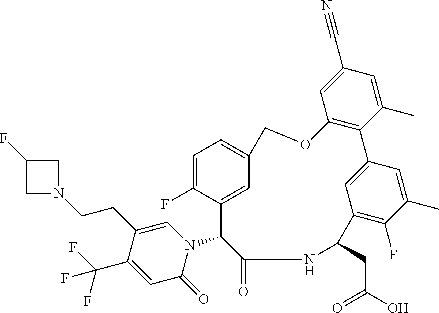 [SMILES: Cc1cc2cc(c1F)[C@@H](CC(=O)O)NC(=O)[C@H](n1cc(CCN3CC(F)C3)c(C(F)(F)F)cc1=O)c1cc(ccc1F)COc1cc(C#N)cc(C)c1-2]

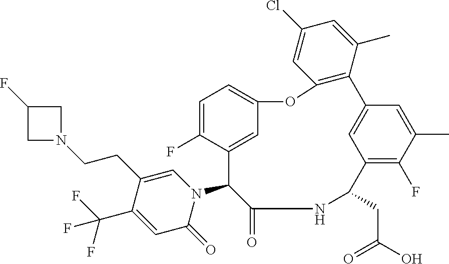 [SMILES: Cc1cc2cc(c1F)[C@H](CC(=O)O)NC(=O)[C@@H](n1cc(CCN3CC(F)C3)c(C(F)(F)F)cc1=O)c1cc(ccc1F)Oc1cc(Cl)cc(C)c1-2]

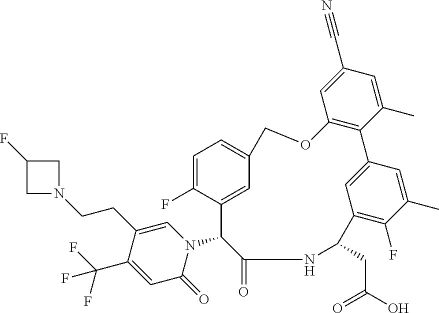 [SMILES: Cc1cc2cc(c1F)[C@H](CC(=O)O)NC(=O)[C@H](n1cc(CCN3CC(F)C3)c(C(F)(F)F)cc1=O)c1cc(ccc1F)COc1cc(C#N)cc(C)c1-2]

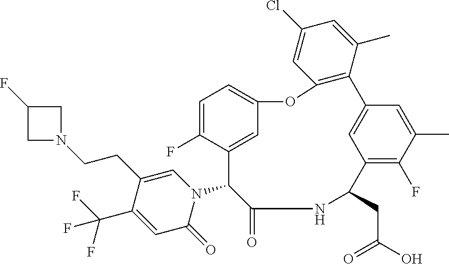 [SMILES: Cc1cc2cc(c1F)[C@@H](CC(=O)O)NC(=O)[C@H](n1cc(CCN3CC(F)C3)c(C(F)(F)F)cc1=O)c1cc(ccc1F)Oc1cc(Cl)cc(C)c1-2]

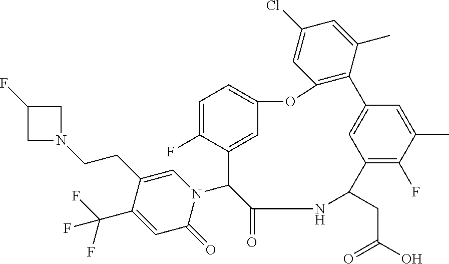 [SMILES: Cc1cc2cc(c1F)C(CC(=O)O)NC(=O)C(n1cc(CCN3CC(F)C3)c(C(F)(F)F)cc1=O)c1cc(ccc1F)Oc1cc(Cl)cc(C)c1-2]

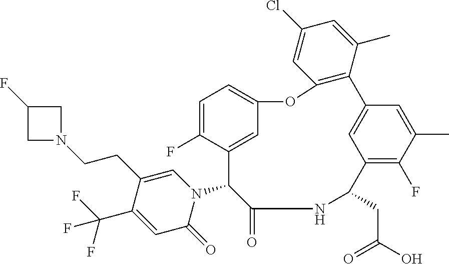 [SMILES: Cc1cc2cc(c1F)[C@H](CC(=O)O)NC(=O)[C@H](n1cc(CCN3CC(F)C3)c(C(F)(F)F)cc1=O)c1cc(ccc1F)Oc1cc(Cl)cc(C)c1-2]

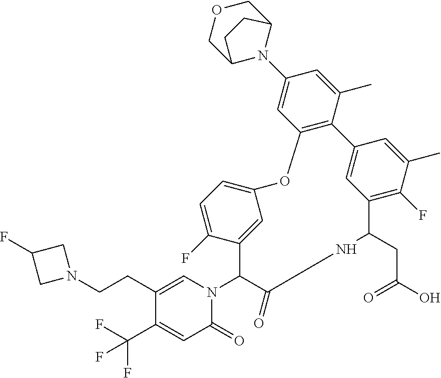 [SMILES: Cc1cc2cc(c1F)C(CC(=O)O)NC(=O)C(n1cc(CCN3CC(F)C3)c(C(F)(F)F)cc1=O)c1cc(ccc1F)Oc1cc(N3C4CCC3COC4)cc(C)c1-2]

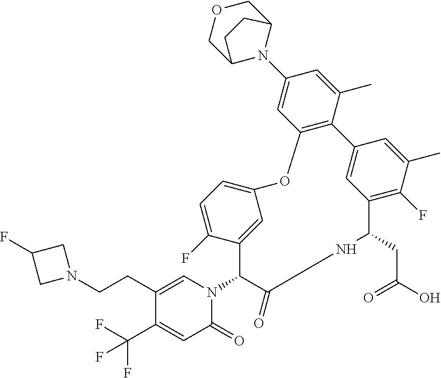 [SMILES: Cc1cc2cc(c1F)[C@H](CC(=O)O)NC(=O)[C@H](n1cc(CCN3CC(F)C3)c(C(F)(F)F)cc1=O)c1cc(ccc1F)Oc1cc(N3C4CCC3COC4)cc(C)c1-2]

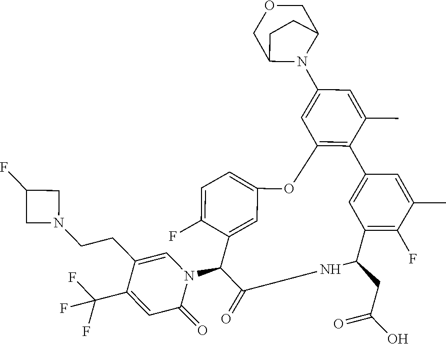 [SMILES: Cc1cc2cc(c1F)[C@@H](CC(=O)O)NC(=O)[C@@H](n1cc(CCN3CC(F)C3)c(C(F)(F)F)cc1=O)c1cc(ccc1F)Oc1cc(N3C4CCC3COC4)cc(C)c1-2]

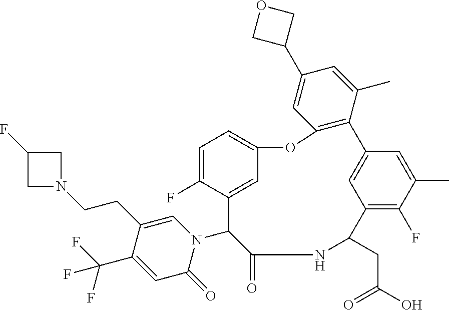 [SMILES: Cc1cc2cc(c1F)C(CC(=O)O)NC(=O)C(n1cc(CCN3CC(F)C3)c(C(F)(F)F)cc1=O)c1cc(ccc1F)Oc1cc(C3COC3)cc(C)c1-2]

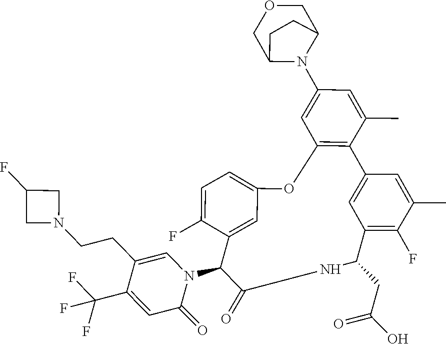 [SMILES: Cc1cc2cc(c1F)[C@H](CC(=O)O)NC(=O)[C@@H](n1cc(CCN3CC(F)C3)c(C(F)(F)F)cc1=O)c1cc(ccc1F)Oc1cc(N3C4CCC3COC4)cc(C)c1-2]

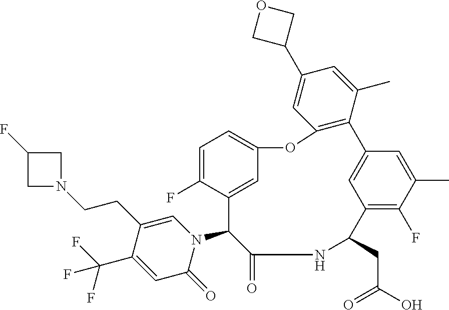 [SMILES: Cc1cc2cc(c1F)[C@@H](CC(=O)O)NC(=O)[C@@H](n1cc(CCN3CC(F)C3)c(C(F)(F)F)cc1=O)c1cc(ccc1F)Oc1cc(C3COC3)cc(C)c1-2]

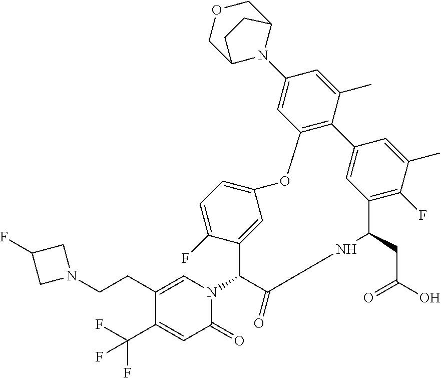 [SMILES: Cc1cc2cc(c1F)[C@@H](CC(=O)O)NC(=O)[C@H](n1cc(CCN3CC(F)C3)c(C(F)(F)F)cc1=O)c1cc(ccc1F)Oc1cc(N3C4CCC3COC4)cc(C)c1-2]

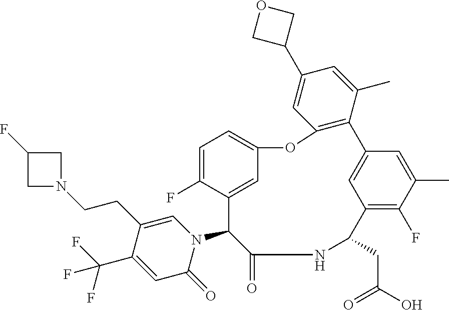 [SMILES: Cc1cc2cc(c1F)[C@H](CC(=O)O)NC(=O)[C@@H](n1cc(CCN3CC(F)C3)c(C(F)(F)F)cc1=O)c1cc(ccc1F)Oc1cc(C3COC3)cc(C)c1-2]

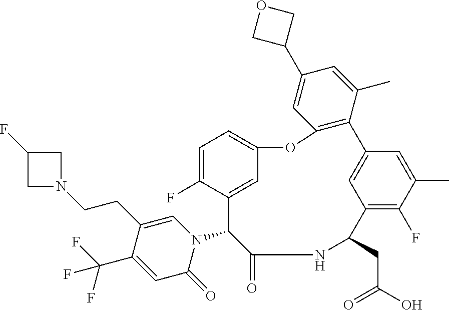 [SMILES: Cc1cc2cc(c1F)[C@@H](CC(=O)O)NC(=O)[C@H](n1cc(CCN3CC(F)C3)c(C(F)(F)F)cc1=O)c1cc(ccc1F)Oc1cc(C3COC3)cc(C)c1-2]

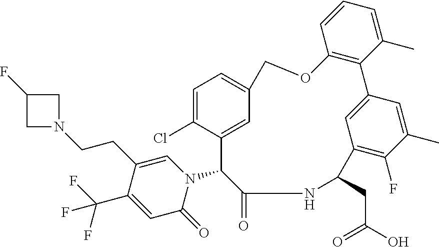 [SMILES: Cc1cc2cc(c1F)[C@@H](CC(=O)O)NC(=O)[C@H](n1cc(CCN3CC(F)C3)c(C(F)(F)F)cc1=O)c1cc(ccc1Cl)COc1cccc(C)c1-2]

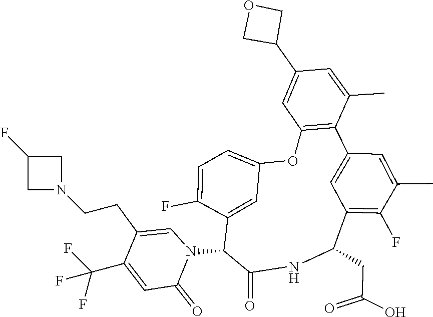 [SMILES: Cc1cc2cc(c1F)[C@H](CC(=O)O)NC(=O)[C@H](n1cc(CCN3CC(F)C3)c(C(F)(F)F)cc1=O)c1cc(ccc1F)Oc1cc(C3COC3)cc(C)c1-2]

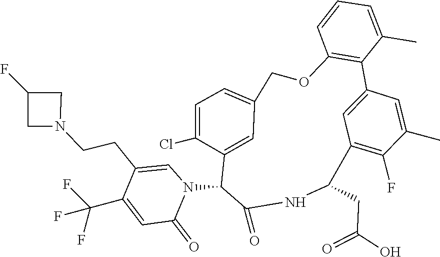 [SMILES: Cc1cc2cc(c1F)[C@H](CC(=O)O)NC(=O)[C@H](n1cc(CCN3CC(F)C3)c(C(F)(F)F)cc1=O)c1cc(ccc1Cl)COc1cccc(C)c1-2]

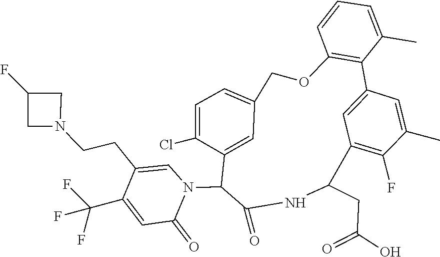 [SMILES: Cc1cc2cc(c1F)C(CC(=O)O)NC(=O)C(n1cc(CCN3CC(F)C3)c(C(F)(F)F)cc1=O)c1cc(ccc1Cl)COc1cccc(C)c1-2]

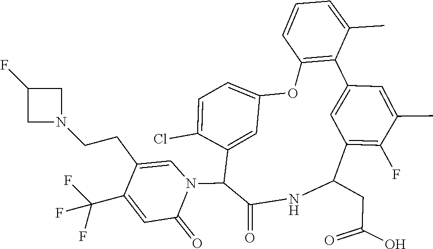 [SMILES: Cc1cc2cc(c1F)C(CC(=O)O)NC(=O)C(n1cc(CCN3CC(F)C3)c(C(F)(F)F)cc1=O)c1cc(ccc1Cl)Oc1cccc(C)c1-2]

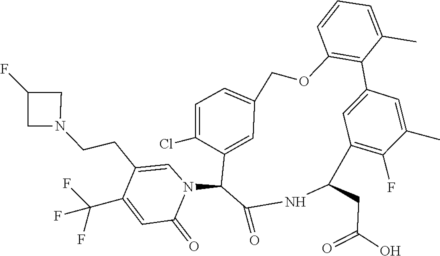 [SMILES: Cc1cc2cc(c1F)[C@@H](CC(=O)O)NC(=O)[C@@H](n1cc(CCN3CC(F)C3)c(C(F)(F)F)cc1=O)c1cc(ccc1Cl)COc1cccc(C)c1-2]

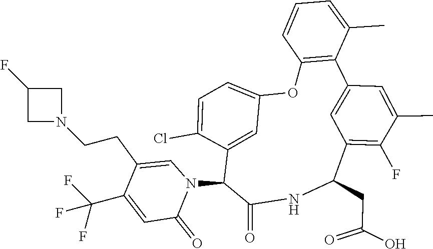 [SMILES: Cc1cc2cc(c1F)[C@@H](CC(=O)O)NC(=O)[C@@H](n1cc(CCN3CC(F)C3)c(C(F)(F)F)cc1=O)c1cc(ccc1Cl)Oc1cccc(C)c1-2]

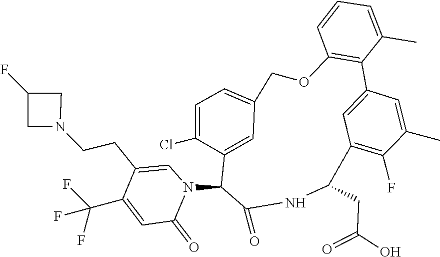 [SMILES: Cc1cc2cc(c1F)[C@H](CC(=O)O)NC(=O)[C@@H](n1cc(CCN3CC(F)C3)c(C(F)(F)F)cc1=O)c1cc(ccc1Cl)COc1cccc(C)c1-2]

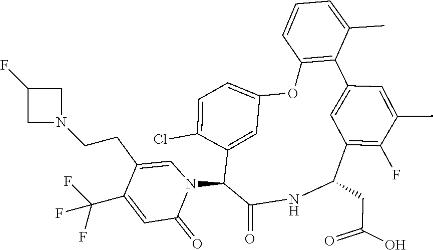 [SMILES: Cc1cc2cc(c1F)[C@H](CC(=O)O)NC(=O)[C@@H](n1cc(CCN3CC(F)C3)c(C(F)(F)F)cc1=O)c1cc(ccc1Cl)Oc1cccc(C)c1-2]

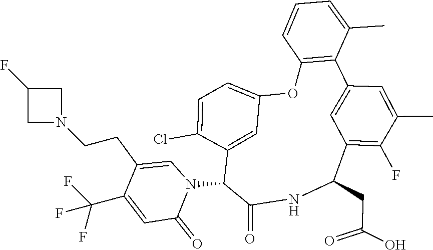 [SMILES: Cc1cc2cc(c1F)[C@@H](CC(=O)O)NC(=O)[C@H](n1cc(CCN3CC(F)C3)c(C(F)(F)F)cc1=O)c1cc(ccc1Cl)Oc1cccc(C)c1-2]

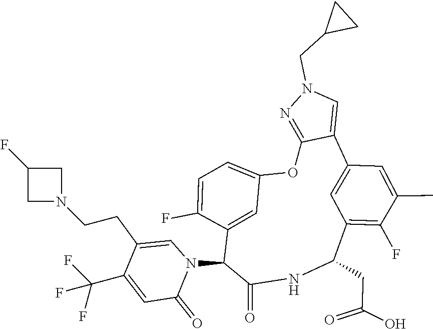 [SMILES: Cc1cc2cc(c1F)[C@H](CC(=O)O)NC(=O)[C@@H](n1cc(CCN3CC(F)C3)c(C(F)(F)F)cc1=O)c1cc(ccc1F)Oc1nn(CC3CC3)cc1-2]

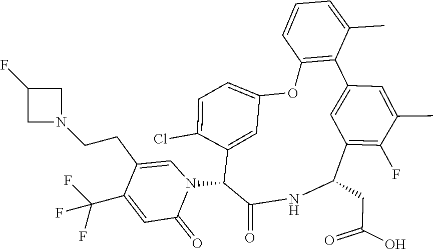 [SMILES: Cc1cc2cc(c1F)[C@H](CC(=O)O)NC(=O)[C@H](n1cc(CCN3CC(F)C3)c(C(F)(F)F)cc1=O)c1cc(ccc1Cl)Oc1cccc(C)c1-2]

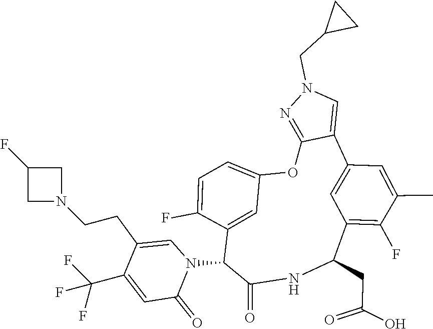 [SMILES: Cc1cc2cc(c1F)[C@@H](CC(=O)O)NC(=O)[C@H](n1cc(CCN3CC(F)C3)c(C(F)(F)F)cc1=O)c1cc(ccc1F)Oc1nn(CC3CC3)cc1-2]

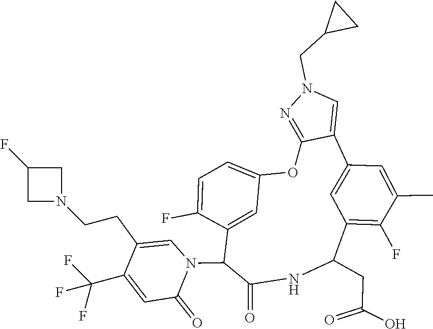 [SMILES: Cc1cc2cc(c1F)C(CC(=O)O)NC(=O)C(n1cc(CCN3CC(F)C3)c(C(F)(F)F)cc1=O)c1cc(ccc1F)Oc1nn(CC3CC3)cc1-2]

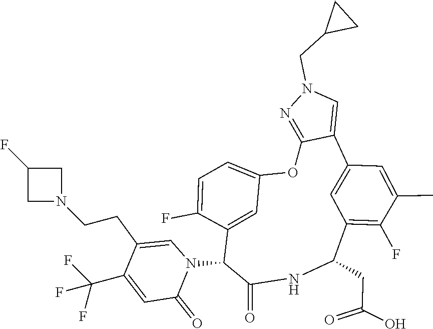 [SMILES: Cc1cc2cc(c1F)[C@H](CC(=O)O)NC(=O)[C@H](n1cc(CCN3CC(F)C3)c(C(F)(F)F)cc1=O)c1cc(ccc1F)Oc1nn(CC3CC3)cc1-2]

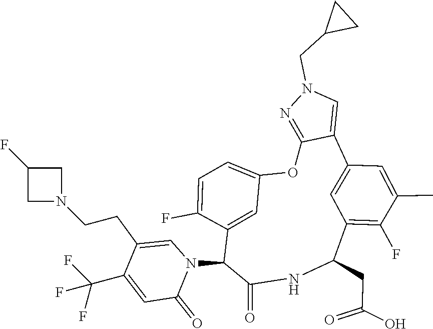 [SMILES: Cc1cc2cc(c1F)[C@@H](CC(=O)O)NC(=O)[C@@H](n1cc(CCN3CC(F)C3)c(C(F)(F)F)cc1=O)c1cc(ccc1F)Oc1nn(CC3CC3)cc1-2]

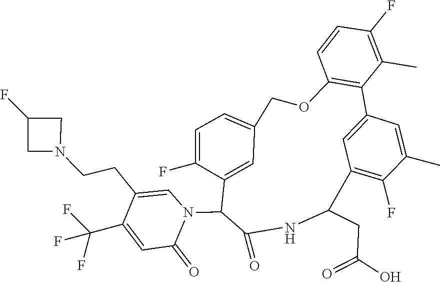 [SMILES: Cc1cc2cc(c1F)C(CC(=O)O)NC(=O)C(n1cc(CCN3CC(F)C3)c(C(F)(F)F)cc1=O)c1cc(ccc1F)COc1ccc(F)c(C)c1-2]

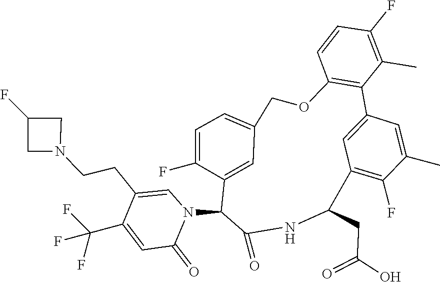 [SMILES: Cc1cc2cc(c1F)[C@@H](CC(=O)O)NC(=O)[C@@H](n1cc(CCN3CC(F)C3)c(C(F)(F)F)cc1=O)c1cc(ccc1F)COc1ccc(F)c(C)c1-2]

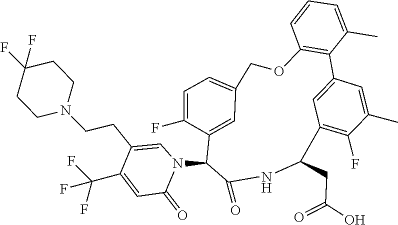 [SMILES: Cc1cc2cc(c1F)[C@@H](CC(=O)O)NC(=O)[C@@H](n1cc(CCN3CCC(F)(F)CC3)c(C(F)(F)F)cc1=O)c1cc(ccc1F)COc1cccc(C)c1-2]

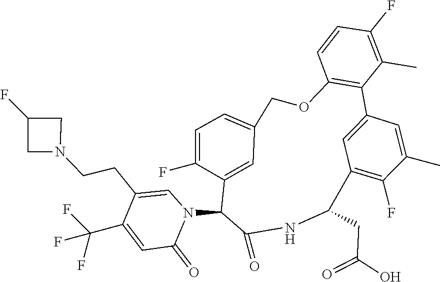 [SMILES: Cc1cc2cc(c1F)[C@H](CC(=O)O)NC(=O)[C@@H](n1cc(CCN3CC(F)C3)c(C(F)(F)F)cc1=O)c1cc(ccc1F)COc1ccc(F)c(C)c1-2]

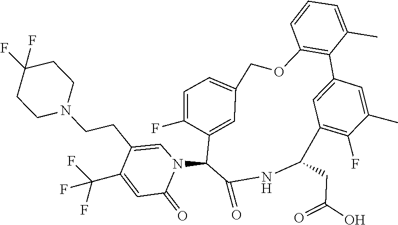 [SMILES: Cc1cc2cc(c1F)[C@H](CC(=O)O)NC(=O)[C@@H](n1cc(CCN3CCC(F)(F)CC3)c(C(F)(F)F)cc1=O)c1cc(ccc1F)COc1cccc(C)c1-2]

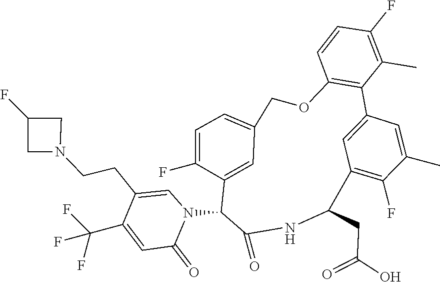 [SMILES: Cc1cc2cc(c1F)[C@@H](CC(=O)O)NC(=O)[C@H](n1cc(CCN3CC(F)C3)c(C(F)(F)F)cc1=O)c1cc(ccc1F)COc1ccc(F)c(C)c1-2]

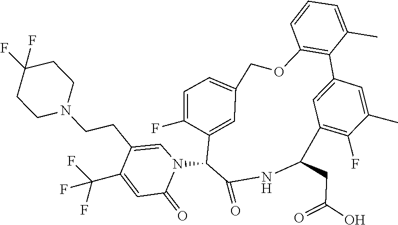 [SMILES: Cc1cc2cc(c1F)[C@@H](CC(=O)O)NC(=O)[C@H](n1cc(CCN3CCC(F)(F)CC3)c(C(F)(F)F)cc1=O)c1cc(ccc1F)COc1cccc(C)c1-2]

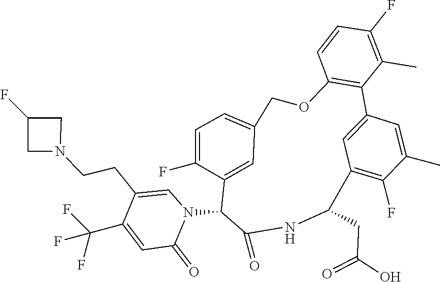 [SMILES: Cc1cc2cc(c1F)[C@H](CC(=O)O)NC(=O)[C@H](n1cc(CCN3CC(F)C3)c(C(F)(F)F)cc1=O)c1cc(ccc1F)COc1ccc(F)c(C)c1-2]

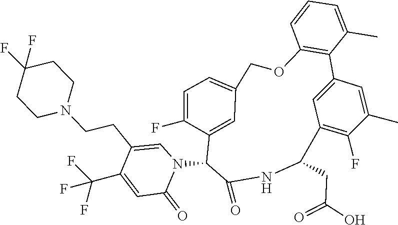 [SMILES: Cc1cc2cc(c1F)[C@H](CC(=O)O)NC(=O)[C@H](n1cc(CCN3CCC(F)(F)CC3)c(C(F)(F)F)cc1=O)c1cc(ccc1F)COc1cccc(C)c1-2]

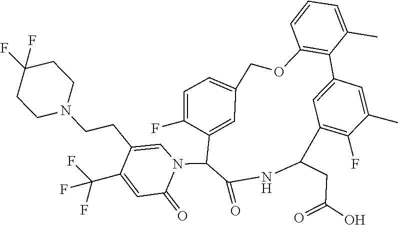 [SMILES: Cc1cc2cc(c1F)C(CC(=O)O)NC(=O)C(n1cc(CCN3CCC(F)(F)CC3)c(C(F)(F)F)cc1=O)c1cc(ccc1F)COc1cccc(C)c1-2]

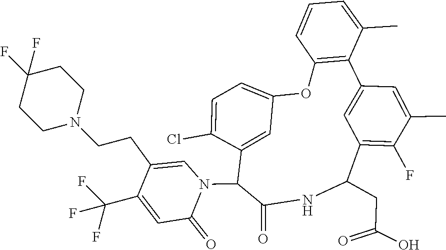 [SMILES: Cc1cc2cc(c1F)C(CC(=O)O)NC(=O)C(n1cc(CCN3CCC(F)(F)CC3)c(C(F)(F)F)cc1=O)c1cc(ccc1Cl)Oc1cccc(C)c1-2]

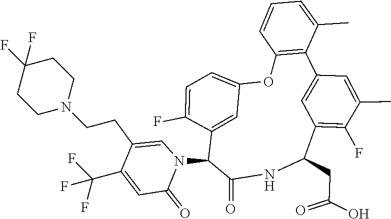 [SMILES: Cc1cc2cc(c1F)[C@@H](CC(=O)O)NC(=O)[C@@H](n1cc(CCN3CCC(F)(F)CC3)c(C(F)(F)F)cc1=O)c1cc(ccc1F)Oc1cccc(C)c1-2]

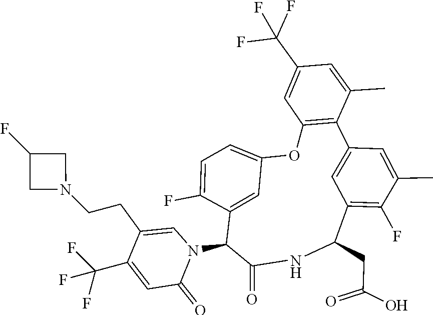 [SMILES: Cc1cc2cc(c1F)[C@@H](CC(=O)O)NC(=O)[C@@H](n1cc(CCN3CC(F)C3)c(C(F)(F)F)cc1=O)c1cc(ccc1F)Oc1cc(C(F)(F)F)cc(C)c1-2]

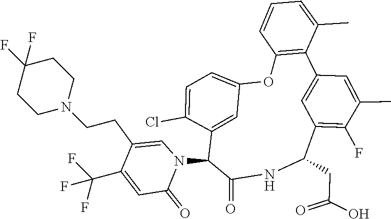 [SMILES: Cc1cc2cc(c1F)[C@H](CC(=O)O)NC(=O)[C@@H](n1cc(CCN3CCC(F)(F)CC3)c(C(F)(F)F)cc1=O)c1cc(ccc1Cl)Oc1cccc(C)c1-2]

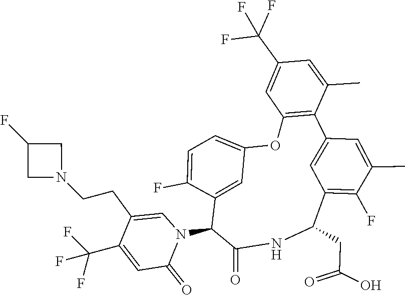 [SMILES: Cc1cc2cc(c1F)[C@H](CC(=O)O)NC(=O)[C@@H](n1cc(CCN3CC(F)C3)c(C(F)(F)F)cc1=O)c1cc(ccc1F)Oc1cc(C(F)(F)F)cc(C)c1-2]

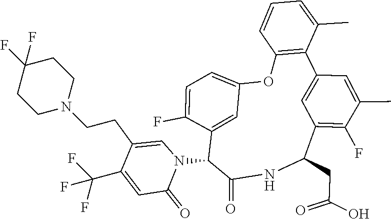 [SMILES: Cc1cc2cc(c1F)[C@@H](CC(=O)O)NC(=O)[C@H](n1cc(CCN3CCC(F)(F)CC3)c(C(F)(F)F)cc1=O)c1cc(ccc1F)Oc1cccc(C)c1-2]

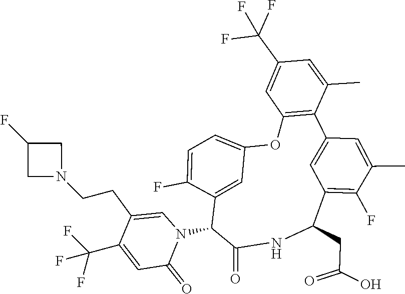 [SMILES: Cc1cc2cc(c1F)[C@@H](CC(=O)O)NC(=O)[C@H](n1cc(CCN3CC(F)C3)c(C(F)(F)F)cc1=O)c1cc(ccc1F)Oc1cc(C(F)(F)F)cc(C)c1-2]

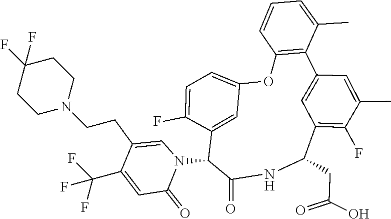 [SMILES: Cc1cc2cc(c1F)[C@H](CC(=O)O)NC(=O)[C@H](n1cc(CCN3CCC(F)(F)CC3)c(C(F)(F)F)cc1=O)c1cc(ccc1F)Oc1cccc(C)c1-2]

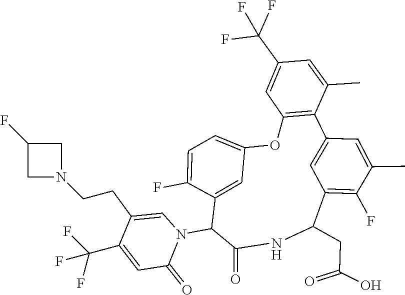 [SMILES: Cc1cc2cc(c1F)C(CC(=O)O)NC(=O)C(n1cc(CCN3CC(F)C3)c(C(F)(F)F)cc1=O)c1cc(ccc1F)Oc1cc(C(F)(F)F)cc(C)c1-2]

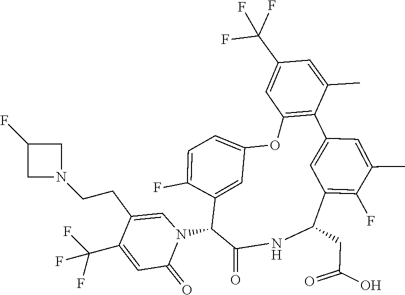 [SMILES: Cc1cc2cc(c1F)[C@H](CC(=O)O)NC(=O)[C@H](n1cc(CCN3CC(F)C3)c(C(F)(F)F)cc1=O)c1cc(ccc1F)Oc1cc(C(F)(F)F)cc(C)c1-2]

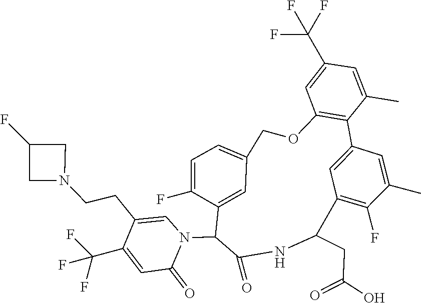 [SMILES: Cc1cc2cc(c1F)C(CC(=O)O)NC(=O)C(n1cc(CCN3CC(F)C3)c(C(F)(F)F)cc1=O)c1cc(ccc1F)COc1cc(C(F)(F)F)cc(C)c1-2]

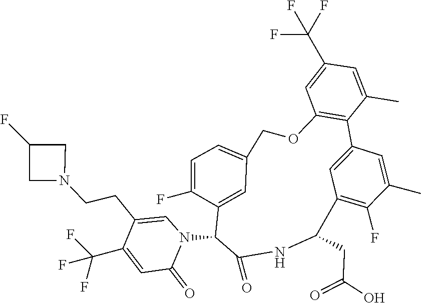 [SMILES: Cc1cc2cc(c1F)[C@H](CC(=O)O)NC(=O)[C@H](n1cc(CCN3CC(F)C3)c(C(F)(F)F)cc1=O)c1cc(ccc1F)COc1cc(C(F)(F)F)cc(C)c1-2]

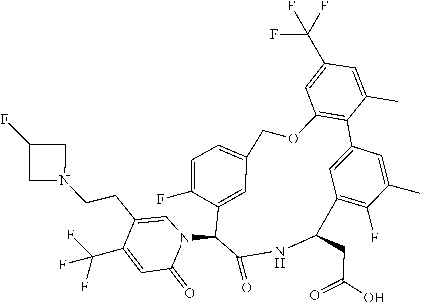 [SMILES: Cc1cc2cc(c1F)[C@@H](CC(=O)O)NC(=O)[C@@H](n1cc(CCN3CC(F)C3)c(C(F)(F)F)cc1=O)c1cc(ccc1F)COc1cc(C(F)(F)F)cc(C)c1-2]

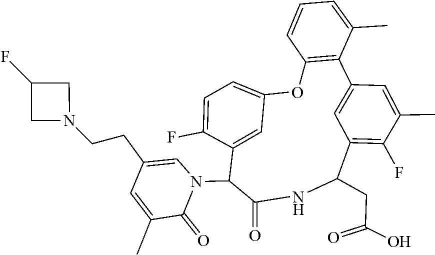 [SMILES: Cc1cc2cc(c1F)C(CC(=O)O)NC(=O)C(n1cc(CCN3CC(F)C3)cc(C)c1=O)c1cc(ccc1F)Oc1cccc(C)c1-2]

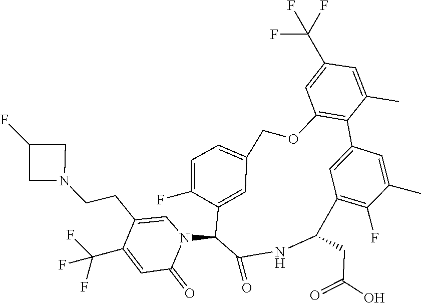 [SMILES: Cc1cc2cc(c1F)[C@H](CC(=O)O)NC(=O)[C@@H](n1cc(CCN3CC(F)C3)c(C(F)(F)F)cc1=O)c1cc(ccc1F)COc1cc(C(F)(F)F)cc(C)c1-2]

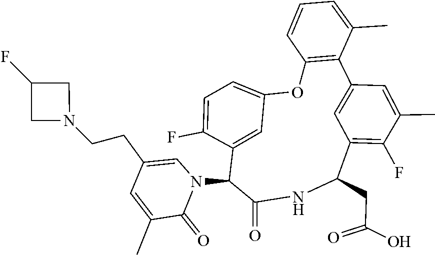 [SMILES: Cc1cc2cc(c1F)[C@@H](CC(=O)O)NC(=O)[C@@H](n1cc(CCN3CC(F)C3)cc(C)c1=O)c1cc(ccc1F)Oc1cccc(C)c1-2]

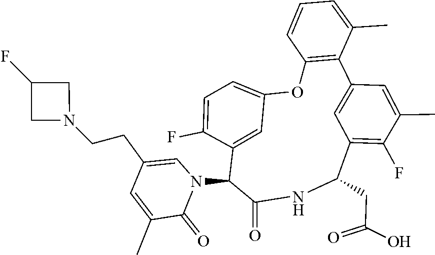 [SMILES: Cc1cc2cc(c1F)[C@H](CC(=O)O)NC(=O)[C@@H](n1cc(CCN3CC(F)C3)cc(C)c1=O)c1cc(ccc1F)Oc1cccc(C)c1-2]

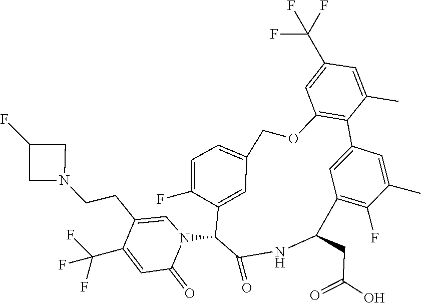 [SMILES: Cc1cc2cc(c1F)[C@@H](CC(=O)O)NC(=O)[C@H](n1cc(CCN3CC(F)C3)c(C(F)(F)F)cc1=O)c1cc(ccc1F)COc1cc(C(F)(F)F)cc(C)c1-2]

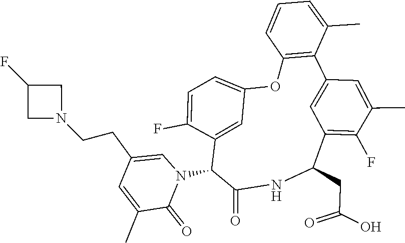 [SMILES: Cc1cc2cc(c1F)[C@@H](CC(=O)O)NC(=O)[C@H](n1cc(CCN3CC(F)C3)cc(C)c1=O)c1cc(ccc1F)Oc1cccc(C)c1-2]

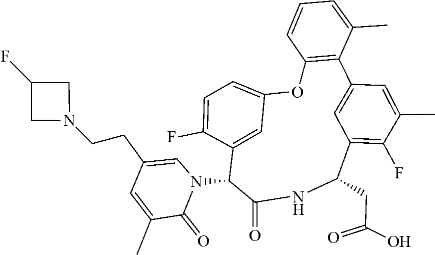 [SMILES: Cc1cc2cc(c1F)[C@H](CC(=O)O)NC(=O)[C@H](n1cc(CCN3CC(F)C3)cc(C)c1=O)c1cc(ccc1F)Oc1cccc(C)c1-2]

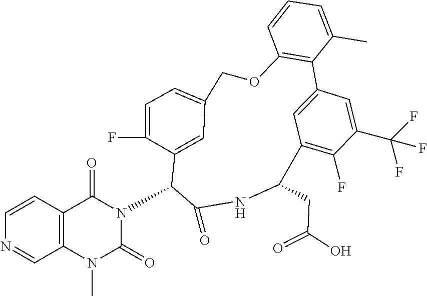 [SMILES: Cc1cccc2c1-c1cc(c(F)c(C(F)(F)F)c1)[C@H](CC(=O)O)NC(=O)[C@H](n1c(=O)c3ccncc3n(C)c1=O)c1cc(ccc1F)CO2]

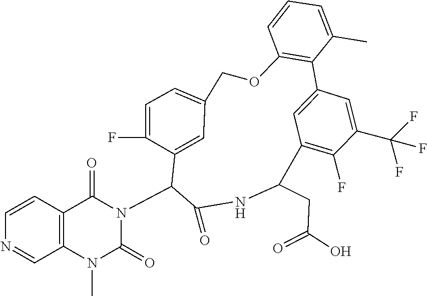 [SMILES: Cc1cccc2c1-c1cc(c(F)c(C(F)(F)F)c1)C(CC(=O)O)NC(=O)C(n1c(=O)c3ccncc3n(C)c1=O)c1cc(ccc1F)CO2]

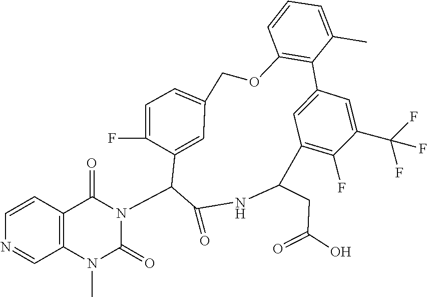 [SMILES: Cc1cccc2c1-c1cc(c(F)c(C(F)(F)F)c1)C(CC(=O)O)NC(=O)C(n1c(=O)c3ccncc3n(C)c1=O)c1cc(ccc1F)CO2]

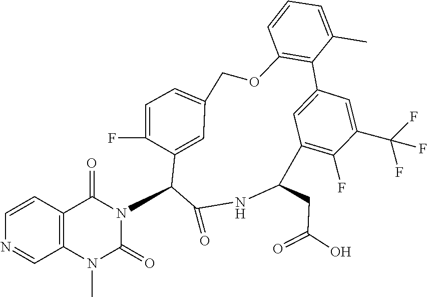 [SMILES: Cc1cccc2c1-c1cc(c(F)c(C(F)(F)F)c1)[C@@H](CC(=O)O)NC(=O)[C@@H](n1c(=O)c3ccncc3n(C)c1=O)c1cc(ccc1F)CO2]

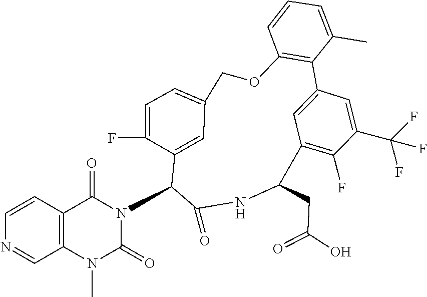 [SMILES: Cc1cccc2c1-c1cc(c(F)c(C(F)(F)F)c1)[C@@H](CC(=O)O)NC(=O)[C@@H](n1c(=O)c3ccncc3n(C)c1=O)c1cc(ccc1F)CO2]

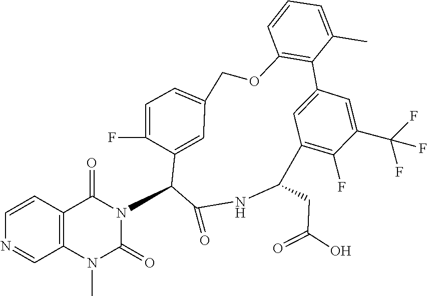 [SMILES: Cc1cccc2c1-c1cc(c(F)c(C(F)(F)F)c1)[C@H](CC(=O)O)NC(=O)[C@@H](n1c(=O)c3ccncc3n(C)c1=O)c1cc(ccc1F)CO2]

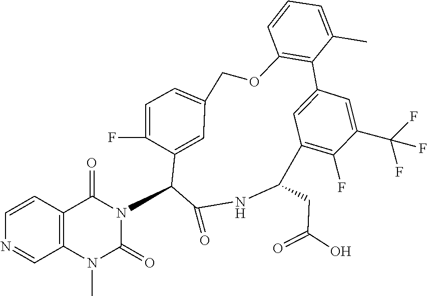 [SMILES: Cc1cccc2c1-c1cc(c(F)c(C(F)(F)F)c1)[C@H](CC(=O)O)NC(=O)[C@@H](n1c(=O)c3ccncc3n(C)c1=O)c1cc(ccc1F)CO2]

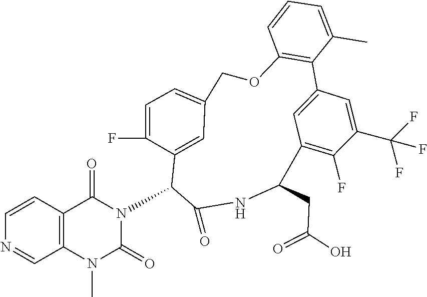 [SMILES: Cc1cccc2c1-c1cc(c(F)c(C(F)(F)F)c1)[C@@H](CC(=O)O)NC(=O)[C@H](n1c(=O)c3ccncc3n(C)c1=O)c1cc(ccc1F)CO2]

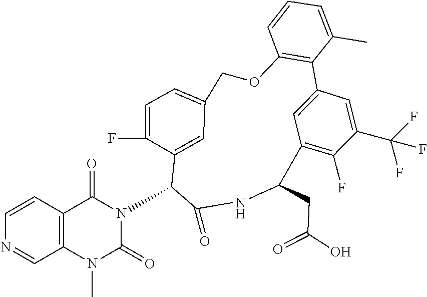 [SMILES: Cc1cccc2c1-c1cc(c(F)c(C(F)(F)F)c1)[C@@H](CC(=O)O)NC(=O)[C@H](n1c(=O)c3ccncc3n(C)c1=O)c1cc(ccc1F)CO2]

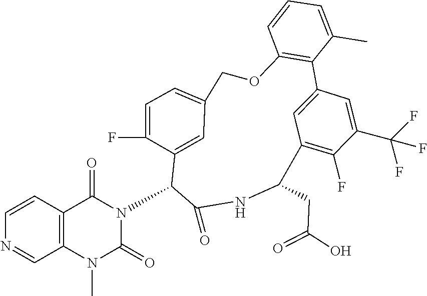 [SMILES: Cc1cccc2c1-c1cc(c(F)c(C(F)(F)F)c1)[C@H](CC(=O)O)NC(=O)[C@H](n1c(=O)c3ccncc3n(C)c1=O)c1cc(ccc1F)CO2]

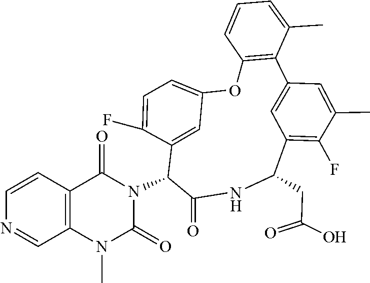 [SMILES: Cc1cc2cc(c1F)[C@H](CC(=O)O)NC(=O)[C@H](n1c(=O)c3ccncc3n(C)c1=O)c1cc(ccc1F)Oc1cccc(C)c1-2]

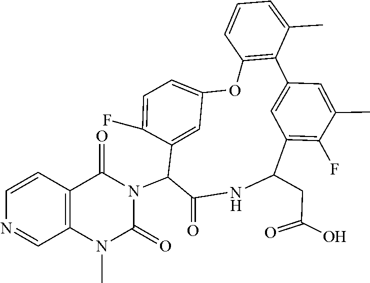 [SMILES: Cc1cc2cc(c1F)C(CC(=O)O)NC(=O)C(n1c(=O)c3ccncc3n(C)c1=O)c1cc(ccc1F)Oc1cccc(C)c1-2]

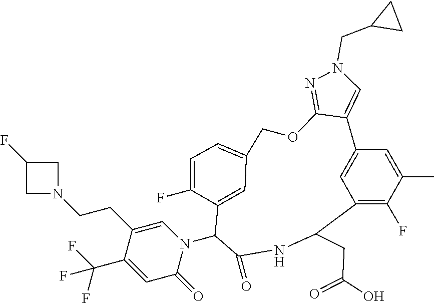 [SMILES: Cc1cc2cc(c1F)C(CC(=O)O)NC(=O)C(n1cc(CCN3CC(F)C3)c(C(F)(F)F)cc1=O)c1cc(ccc1F)COc1nn(CC3CC3)cc1-2]

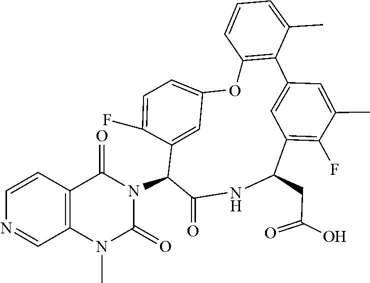 [SMILES: Cc1cc2cc(c1F)[C@@H](CC(=O)O)NC(=O)[C@@H](n1c(=O)c3ccncc3n(C)c1=O)c1cc(ccc1F)Oc1cccc(C)c1-2]

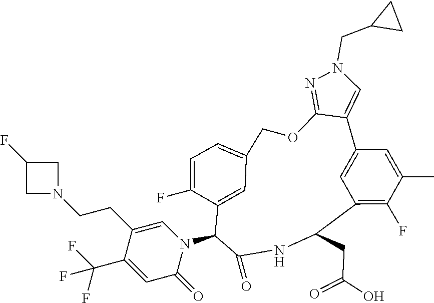 [SMILES: Cc1cc2cc(c1F)[C@@H](CC(=O)O)NC(=O)[C@@H](n1cc(CCN3CC(F)C3)c(C(F)(F)F)cc1=O)c1cc(ccc1F)COc1nn(CC3CC3)cc1-2]

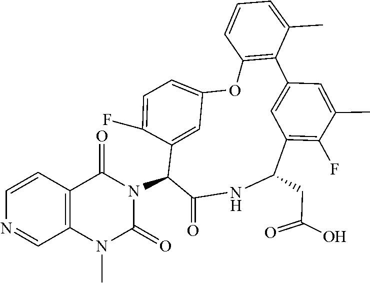 [SMILES: Cc1cc2cc(c1F)[C@H](CC(=O)O)NC(=O)[C@@H](n1c(=O)c3ccncc3n(C)c1=O)c1cc(ccc1F)Oc1cccc(C)c1-2]

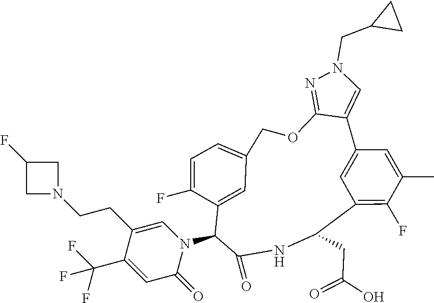 [SMILES: Cc1cc2cc(c1F)[C@H](CC(=O)O)NC(=O)[C@@H](n1cc(CCN3CC(F)C3)c(C(F)(F)F)cc1=O)c1cc(ccc1F)COc1nn(CC3CC3)cc1-2]

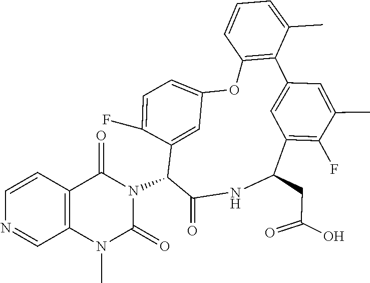 [SMILES: Cc1cc2cc(c1F)[C@@H](CC(=O)O)NC(=O)[C@H](n1c(=O)c3ccncc3n(C)c1=O)c1cc(ccc1F)Oc1cccc(C)c1-2]

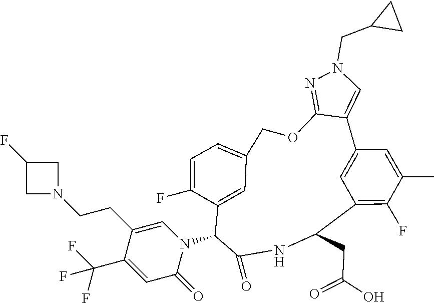 [SMILES: Cc1cc2cc(c1F)[C@@H](CC(=O)O)NC(=O)[C@H](n1cc(CCN3CC(F)C3)c(C(F)(F)F)cc1=O)c1cc(ccc1F)COc1nn(CC3CC3)cc1-2]

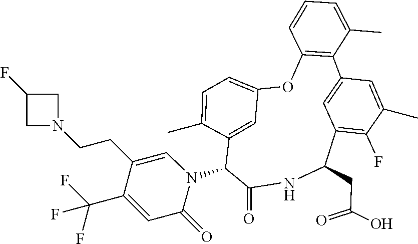 [SMILES: Cc1ccc2cc1[C@@H](n1cc(CCN3CC(F)C3)c(C(F)(F)F)cc1=O)C(=O)N[C@H](CC(=O)O)c1cc(cc(C)c1F)-c1c(C)cccc1O2]

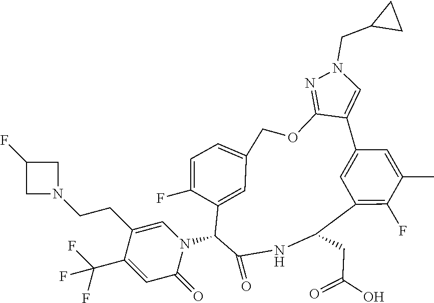 [SMILES: Cc1cc2cc(c1F)[C@H](CC(=O)O)NC(=O)[C@H](n1cc(CCN3CC(F)C3)c(C(F)(F)F)cc1=O)c1cc(ccc1F)COc1nn(CC3CC3)cc1-2]

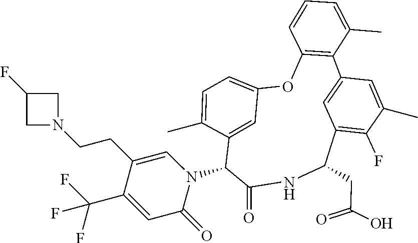 [SMILES: Cc1ccc2cc1[C@@H](n1cc(CCN3CC(F)C3)c(C(F)(F)F)cc1=O)C(=O)N[C@@H](CC(=O)O)c1cc(cc(C)c1F)-c1c(C)cccc1O2]

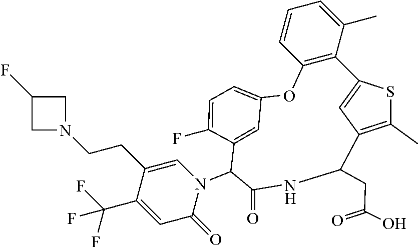 [SMILES: Cc1cccc2c1-c1cc(c(C)s1)C(CC(=O)O)NC(=O)C(n1cc(CCN3CC(F)C3)c(C(F)(F)F)cc1=O)c1cc(ccc1F)O2]

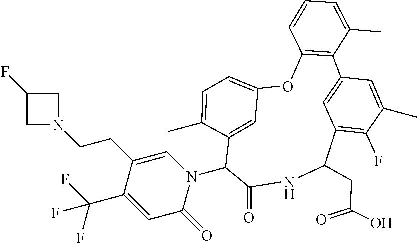 [SMILES: Cc1ccc2cc1C(n1cc(CCN3CC(F)C3)c(C(F)(F)F)cc1=O)C(=O)NC(CC(=O)O)c1cc(cc(C)c1F)-c1c(C)cccc1O2]

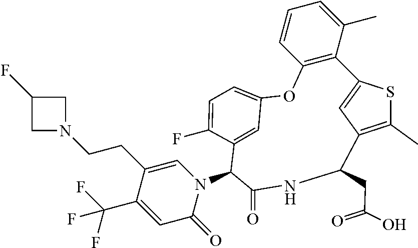 [SMILES: Cc1cccc2c1-c1cc(c(C)s1)[C@@H](CC(=O)O)NC(=O)[C@@H](n1cc(CCN3CC(F)C3)c(C(F)(F)F)cc1=O)c1cc(ccc1F)O2]

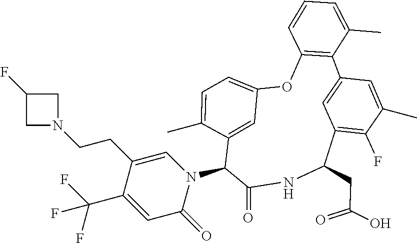 [SMILES: Cc1ccc2cc1[C@H](n1cc(CCN3CC(F)C3)c(C(F)(F)F)cc1=O)C(=O)N[C@H](CC(=O)O)c1cc(cc(C)c1F)-c1c(C)cccc1O2]

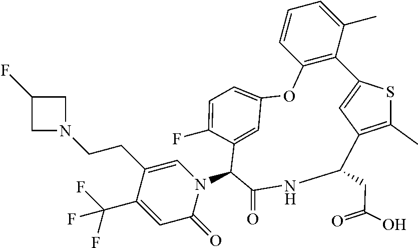 [SMILES: Cc1cccc2c1-c1cc(c(C)s1)[C@H](CC(=O)O)NC(=O)[C@@H](n1cc(CCN3CC(F)C3)c(C(F)(F)F)cc1=O)c1cc(ccc1F)O2]

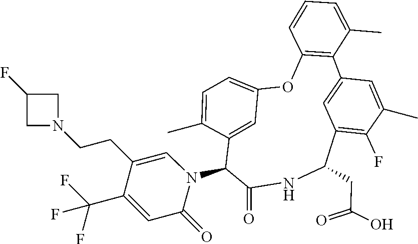 [SMILES: Cc1ccc2cc1[C@H](n1cc(CCN3CC(F)C3)c(C(F)(F)F)cc1=O)C(=O)N[C@@H](CC(=O)O)c1cc(cc(C)c1F)-c1c(C)cccc1O2]

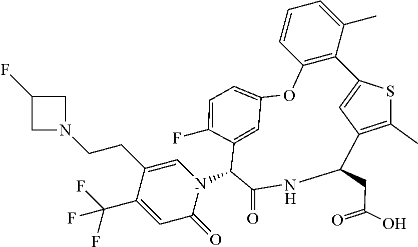 [SMILES: Cc1cccc2c1-c1cc(c(C)s1)[C@@H](CC(=O)O)NC(=O)[C@H](n1cc(CCN3CC(F)C3)c(C(F)(F)F)cc1=O)c1cc(ccc1F)O2]

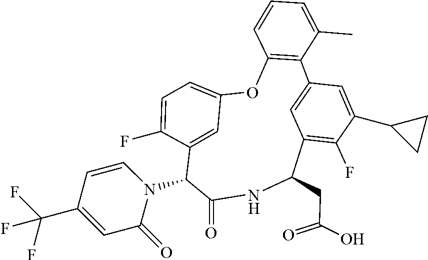 [SMILES: Cc1cccc2c1-c1cc(C3CC3)c(F)c(c1)[C@@H](CC(=O)O)NC(=O)[C@H](n1ccc(C(F)(F)F)cc1=O)c1cc(ccc1F)O2]

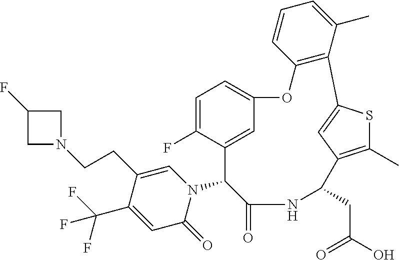 [SMILES: Cc1cccc2c1-c1cc(c(C)s1)[C@H](CC(=O)O)NC(=O)[C@H](n1cc(CCN3CC(F)C3)c(C(F)(F)F)cc1=O)c1cc(ccc1F)O2]

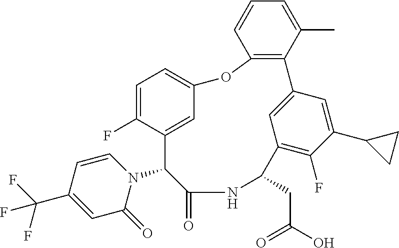 [SMILES: Cc1cccc2c1-c1cc(C3CC3)c(F)c(c1)[C@H](CC(=O)O)NC(=O)[C@H](n1ccc(C(F)(F)F)cc1=O)c1cc(ccc1F)O2]

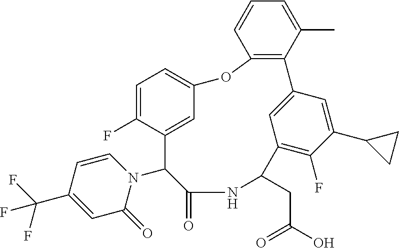 [SMILES: Cc1cccc2c1-c1cc(C3CC3)c(F)c(c1)C(CC(=O)O)NC(=O)C(n1ccc(C(F)(F)F)cc1=O)c1cc(ccc1F)O2]

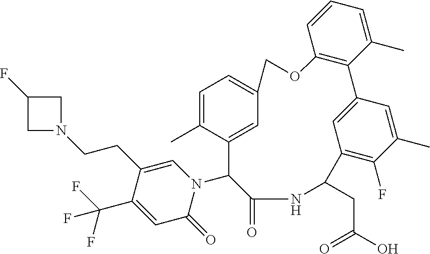 [SMILES: Cc1ccc2cc1C(n1cc(CCN3CC(F)C3)c(C(F)(F)F)cc1=O)C(=O)NC(CC(=O)O)c1cc(cc(C)c1F)-c1c(C)cccc1OC2]

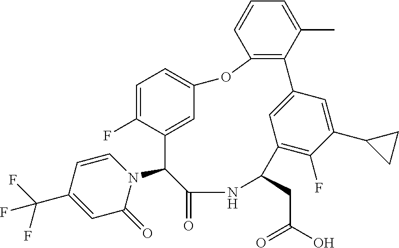 [SMILES: Cc1cccc2c1-c1cc(C3CC3)c(F)c(c1)[C@@H](CC(=O)O)NC(=O)[C@@H](n1ccc(C(F)(F)F)cc1=O)c1cc(ccc1F)O2]

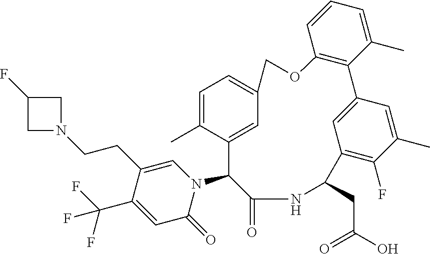 [SMILES: Cc1ccc2cc1[C@H](n1cc(CCN3CC(F)C3)c(C(F)(F)F)cc1=O)C(=O)N[C@H](CC(=O)O)c1cc(cc(C)c1F)-c1c(C)cccc1OC2]

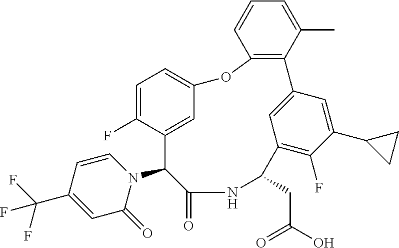 [SMILES: Cc1cccc2c1-c1cc(C3CC3)c(F)c(c1)[C@H](CC(=O)O)NC(=O)[C@@H](n1ccc(C(F)(F)F)cc1=O)c1cc(ccc1F)O2]

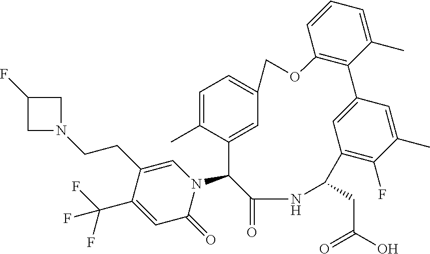 [SMILES: Cc1ccc2cc1[C@H](n1cc(CCN3CC(F)C3)c(C(F)(F)F)cc1=O)C(=O)N[C@@H](CC(=O)O)c1cc(cc(C)c1F)-c1c(C)cccc1OC2]

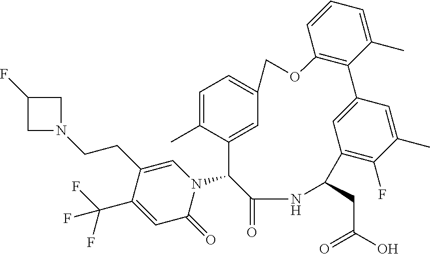 [SMILES: Cc1ccc2cc1[C@@H](n1cc(CCN3CC(F)C3)c(C(F)(F)F)cc1=O)C(=O)N[C@H](CC(=O)O)c1cc(cc(C)c1F)-c1c(C)cccc1OC2]

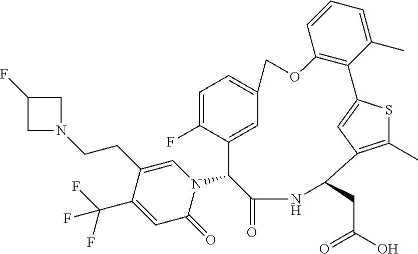 [SMILES: Cc1cccc2c1-c1cc(c(C)s1)[C@@H](CC(=O)O)NC(=O)[C@H](n1cc(CCN3CC(F)C3)c(C(F)(F)F)cc1=O)c1cc(ccc1F)CO2]

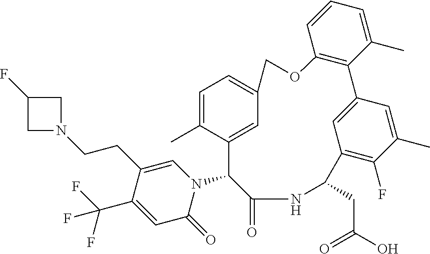 [SMILES: Cc1ccc2cc1[C@@H](n1cc(CCN3CC(F)C3)c(C(F)(F)F)cc1=O)C(=O)N[C@@H](CC(=O)O)c1cc(cc(C)c1F)-c1c(C)cccc1OC2]

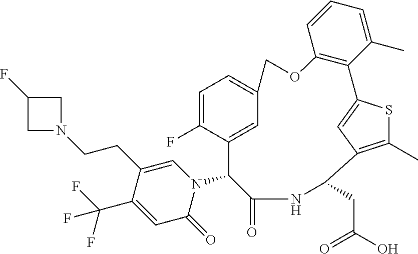 [SMILES: Cc1cccc2c1-c1cc(c(C)s1)[C@H](CC(=O)O)NC(=O)[C@H](n1cc(CCN3CC(F)C3)c(C(F)(F)F)cc1=O)c1cc(ccc1F)CO2]

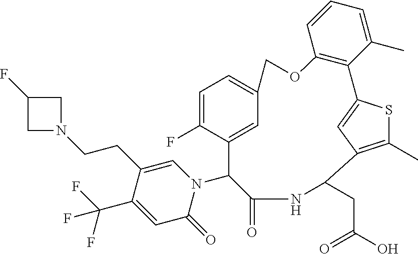 [SMILES: Cc1cccc2c1-c1cc(c(C)s1)C(CC(=O)O)NC(=O)C(n1cc(CCN3CC(F)C3)c(C(F)(F)F)cc1=O)c1cc(ccc1F)CO2]

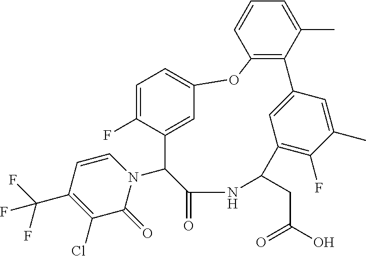 [SMILES: Cc1cc2cc(c1F)C(CC(=O)O)NC(=O)C(n1ccc(C(F)(F)F)c(Cl)c1=O)c1cc(ccc1F)Oc1cccc(C)c1-2]

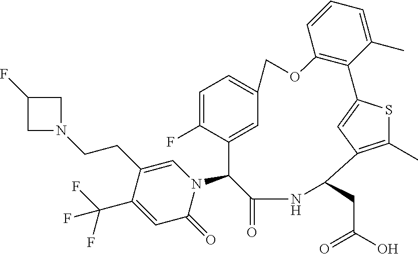 [SMILES: Cc1cccc2c1-c1cc(c(C)s1)[C@@H](CC(=O)O)NC(=O)[C@@H](n1cc(CCN3CC(F)C3)c(C(F)(F)F)cc1=O)c1cc(ccc1F)CO2]

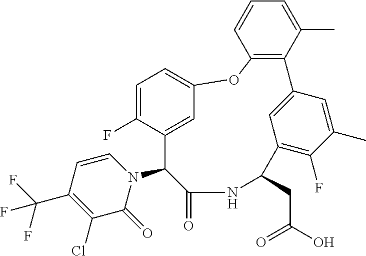 [SMILES: Cc1cc2cc(c1F)[C@@H](CC(=O)O)NC(=O)[C@@H](n1ccc(C(F)(F)F)c(Cl)c1=O)c1cc(ccc1F)Oc1cccc(C)c1-2]

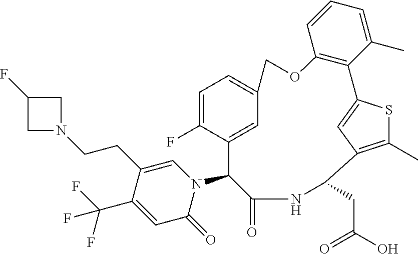 [SMILES: Cc1cccc2c1-c1cc(c(C)s1)[C@H](CC(=O)O)NC(=O)[C@@H](n1cc(CCN3CC(F)C3)c(C(F)(F)F)cc1=O)c1cc(ccc1F)CO2]

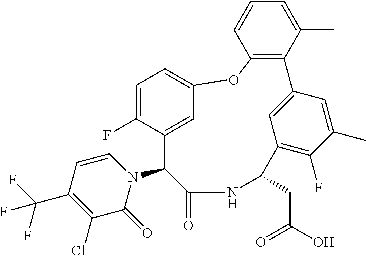 [SMILES: Cc1cc2cc(c1F)[C@H](CC(=O)O)NC(=O)[C@@H](n1ccc(C(F)(F)F)c(Cl)c1=O)c1cc(ccc1F)Oc1cccc(C)c1-2]

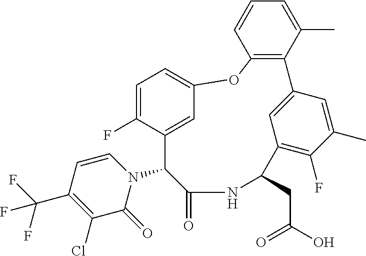 [SMILES: Cc1cc2cc(c1F)[C@@H](CC(=O)O)NC(=O)[C@H](n1ccc(C(F)(F)F)c(Cl)c1=O)c1cc(ccc1F)Oc1cccc(C)c1-2]

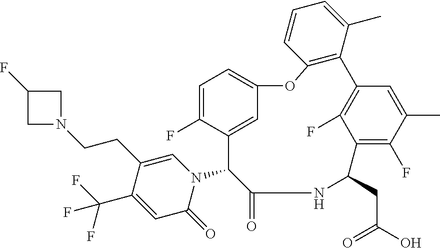 [SMILES: Cc1cc2c(F)c(c1F)[C@@H](CC(=O)O)NC(=O)[C@H](n1cc(CCN3CC(F)C3)c(C(F)(F)F)cc1=O)c1cc(ccc1F)Oc1cccc(C)c1-2]

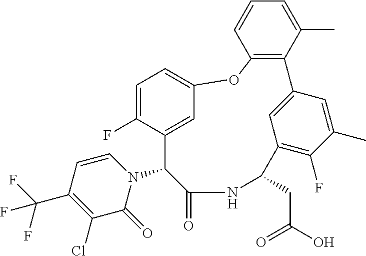 [SMILES: Cc1cc2cc(c1F)[C@H](CC(=O)O)NC(=O)[C@H](n1ccc(C(F)(F)F)c(Cl)c1=O)c1cc(ccc1F)Oc1cccc(C)c1-2]

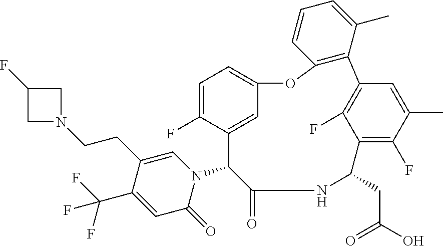 [SMILES: Cc1cc2c(F)c(c1F)[C@H](CC(=O)O)NC(=O)[C@H](n1cc(CCN3CC(F)C3)c(C(F)(F)F)cc1=O)c1cc(ccc1F)Oc1cccc(C)c1-2]

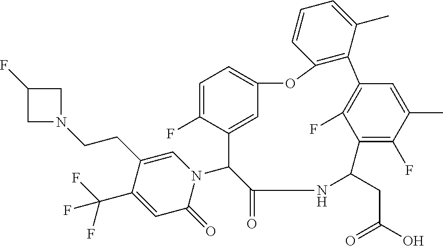 [SMILES: Cc1cc2c(F)c(c1F)C(CC(=O)O)NC(=O)C(n1cc(CCN3CC(F)C3)c(C(F)(F)F)cc1=O)c1cc(ccc1F)Oc1cccc(C)c1-2]

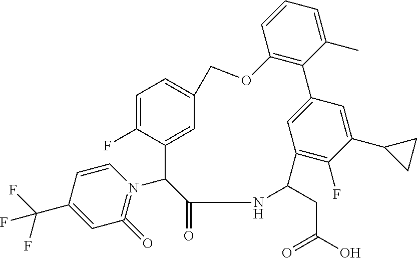

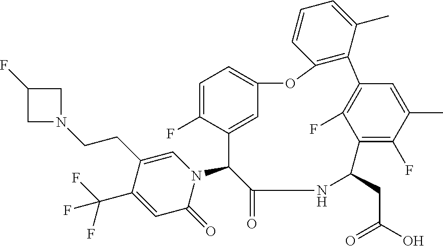 [SMILES: Cc1cc2c(F)c(c1F)[C@@H](CC(=O)O)NC(=O)[C@@H](n1cc(CCN3CC(F)C3)c(C(F)(F)F)cc1=O)c1cc(ccc1F)Oc1cccc(C)c1-2]

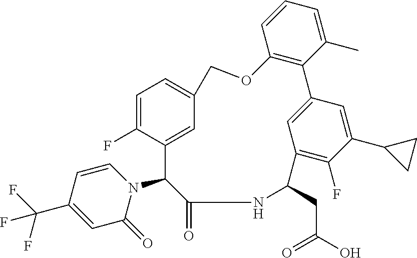 [SMILES: Cc1cccc2c1-c1cc(C3CC3)c(F)c(c1)[C@@H](CC(=O)O)NC(=O)[C@@H](n1ccc(C(F)(F)F)cc1=O)c1cc(ccc1F)CO2]

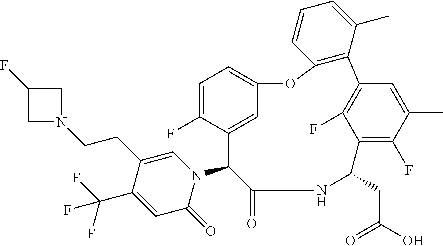 [SMILES: Cc1cc2c(F)c(c1F)[C@H](CC(=O)O)NC(=O)[C@@H](n1cc(CCN3CC(F)C3)c(C(F)(F)F)cc1=O)c1cc(ccc1F)Oc1cccc(C)c1-2]

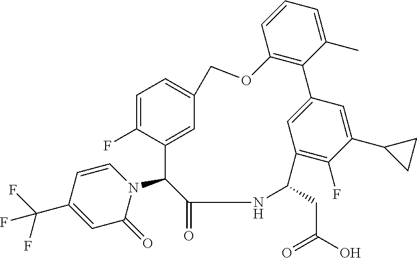 [SMILES: Cc1cccc2c1-c1cc(C3CC3)c(F)c(c1)[C@H](CC(=O)O)NC(=O)[C@@H](n1ccc(C(F)(F)F)cc1=O)c1cc(ccc1F)CO2]

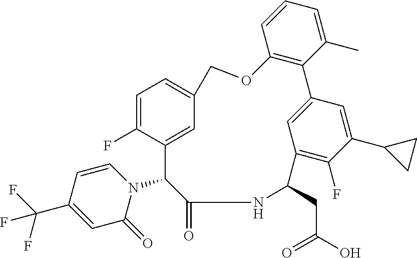 [SMILES: Cc1cccc2c1-c1cc(C3CC3)c(F)c(c1)[C@@H](CC(=O)O)NC(=O)[C@H](n1ccc(C(F)(F)F)cc1=O)c1cc(ccc1F)CO2]

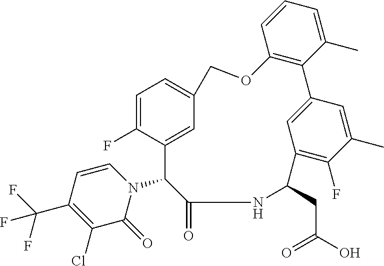 [SMILES: Cc1cc2cc(c1F)[C@@H](CC(=O)O)NC(=O)[C@H](n1ccc(C(F)(F)F)c(Cl)c1=O)c1cc(ccc1F)COc1cccc(C)c1-2]

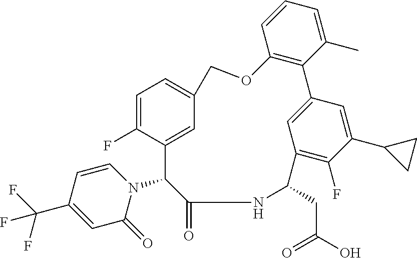 [SMILES: Cc1cccc2c1-c1cc(C3CC3)c(F)c(c1)[C@H](CC(=O)O)NC(=O)[C@H](n1ccc(C(F)(F)F)cc1=O)c1cc(ccc1F)CO2]

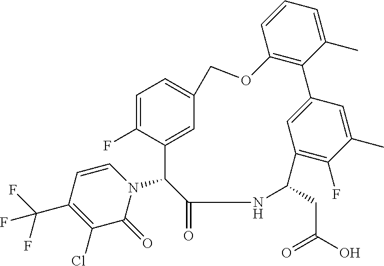 [SMILES: Cc1cc2cc(c1F)[C@H](CC(=O)O)NC(=O)[C@H](n1ccc(C(F)(F)F)c(Cl)c1=O)c1cc(ccc1F)COc1cccc(C)c1-2]

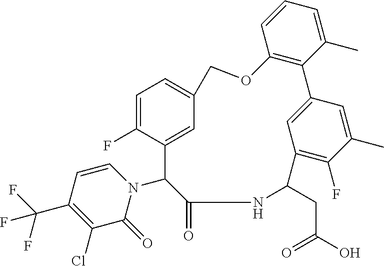 [SMILES: Cc1cc2cc(c1F)C(CC(=O)O)NC(=O)C(n1ccc(C(F)(F)F)c(Cl)c1=O)c1cc(ccc1F)COc1cccc(C)c1-2]

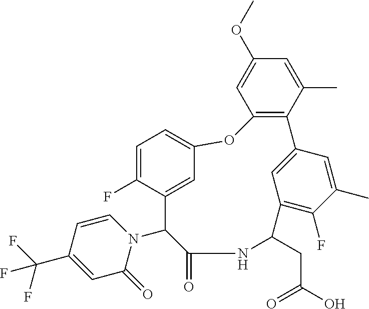 [SMILES: COc1cc(C)c2c(c1)Oc1ccc(F)c(c1)C(n1ccc(C(F)(F)F)cc1=O)C(=O)NC(CC(=O)O)c1cc-2cc(C)c1F]

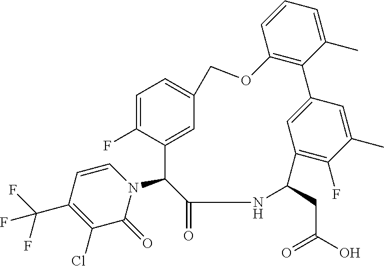 [SMILES: Cc1cc2cc(c1F)[C@@H](CC(=O)O)NC(=O)[C@@H](n1ccc(C(F)(F)F)c(Cl)c1=O)c1cc(ccc1F)COc1cccc(C)c1-2]

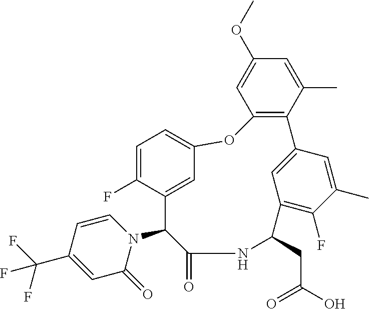 [SMILES: COc1cc(C)c2c(c1)Oc1ccc(F)c(c1)[C@H](n1ccc(C(F)(F)F)cc1=O)C(=O)N[C@H](CC(=O)O)c1cc-2cc(C)c1F]

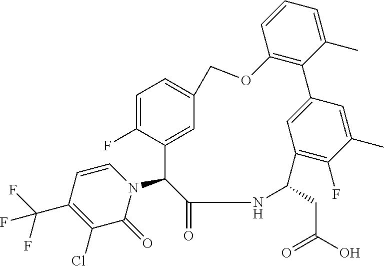 [SMILES: Cc1cc2cc(c1F)[C@H](CC(=O)O)NC(=O)[C@@H](n1ccc(C(F)(F)F)c(Cl)c1=O)c1cc(ccc1F)COc1cccc(C)c1-2]

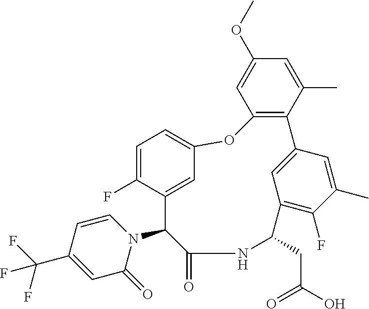 [SMILES: COc1cc(C)c2c(c1)Oc1ccc(F)c(c1)[C@H](n1ccc(C(F)(F)F)cc1=O)C(=O)N[C@@H](CC(=O)O)c1cc-2cc(C)c1F]

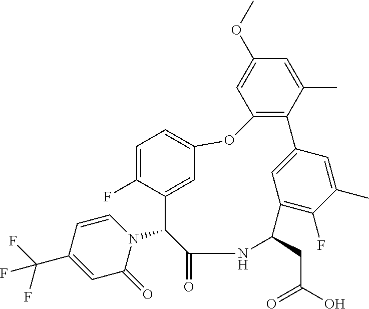 [SMILES: COc1cc(C)c2c(c1)Oc1ccc(F)c(c1)[C@@H](n1ccc(C(F)(F)F)cc1=O)C(=O)N[C@H](CC(=O)O)c1cc-2cc(C)c1F]

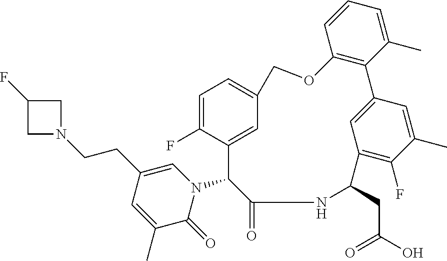 [SMILES: Cc1cc2cc(c1F)[C@@H](CC(=O)O)NC(=O)[C@H](n1cc(CCN3CC(F)C3)cc(C)c1=O)c1cc(ccc1F)COc1cccc(C)c1-2]

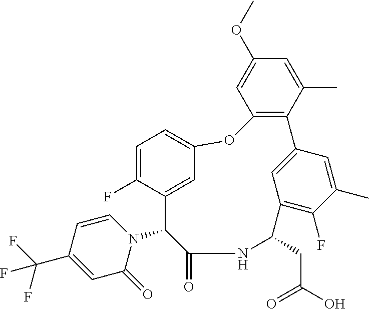 [SMILES: COc1cc(C)c2c(c1)Oc1ccc(F)c(c1)[C@@H](n1ccc(C(F)(F)F)cc1=O)C(=O)N[C@@H](CC(=O)O)c1cc-2cc(C)c1F]

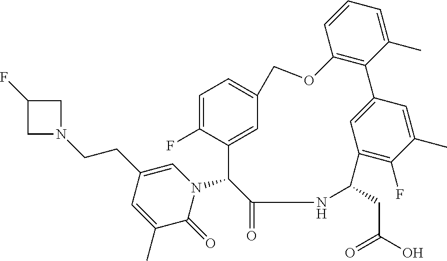 [SMILES: Cc1cc2cc(c1F)[C@H](CC(=O)O)NC(=O)[C@H](n1cc(CCN3CC(F)C3)cc(C)c1=O)c1cc(ccc1F)COc1cccc(C)c1-2]

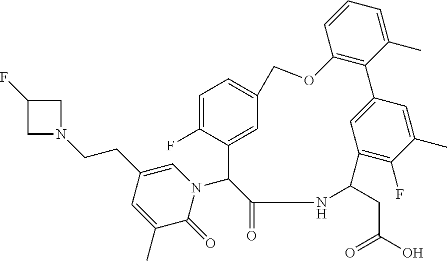 [SMILES: Cc1cc2cc(c1F)C(CC(=O)O)NC(=O)C(n1cc(CCN3CC(F)C3)cc(C)c1=O)c1cc(ccc1F)COc1cccc(C)c1-2]

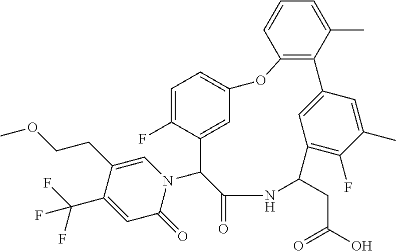

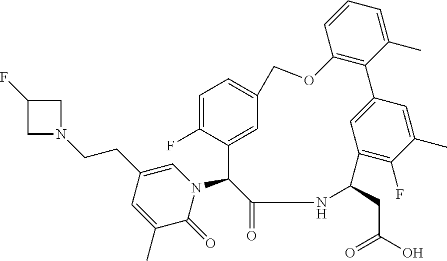 [SMILES: Cc1cc2cc(c1F)[C@@H](CC(=O)O)NC(=O)[C@@H](n1cc(CCN3CC(F)C3)cc(C)c1=O)c1cc(ccc1F)COc1cccc(C)c1-2]

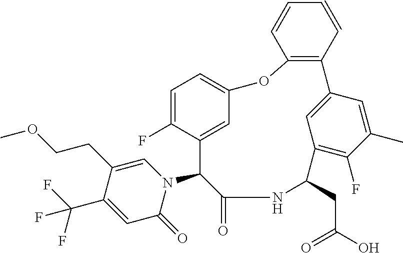

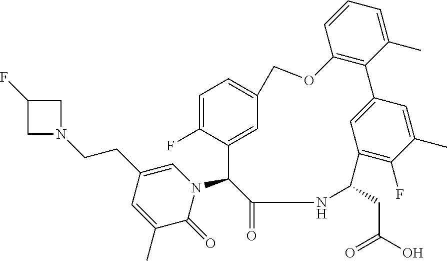 [SMILES: Cc1cc2cc(c1F)[C@H](CC(=O)O)NC(=O)[C@@H](n1cc(CCN3CC(F)C3)cc(C)c1=O)c1cc(ccc1F)COc1cccc(C)c1-2]

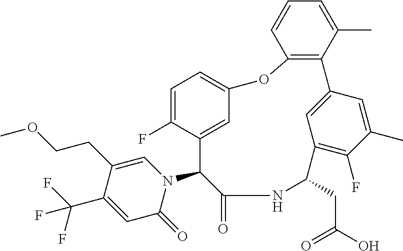

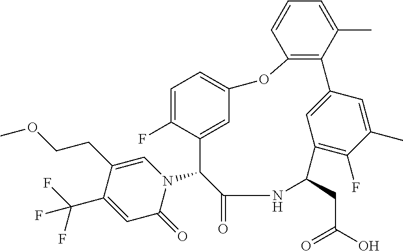

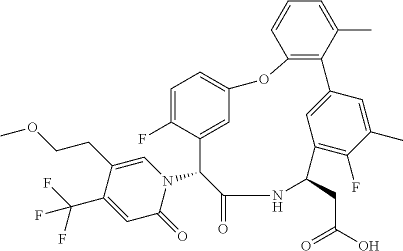

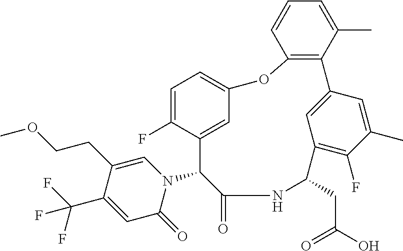

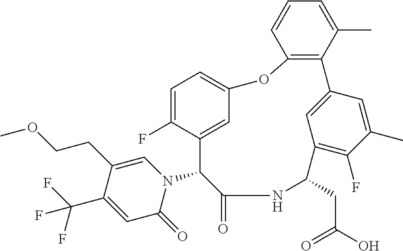

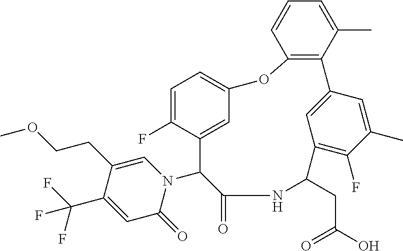

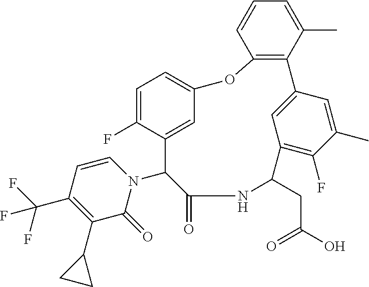 [SMILES: Cc1cc2cc(c1F)C(CC(=O)O)NC(=O)C(n1ccc(C(F)(F)F)c(C3CC3)c1=O)c1cc(ccc1F)Oc1cccc(C)c1-2]

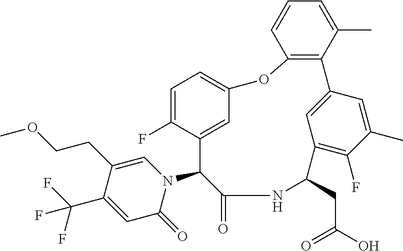

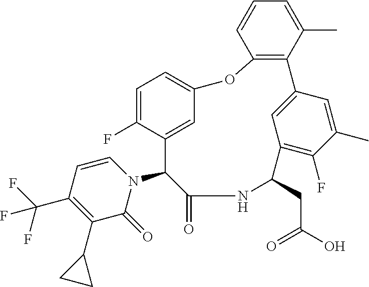 [SMILES: Cc1cc2cc(c1F)[C@@H](CC(=O)O)NC(=O)[C@@H](n1ccc(C(F)(F)F)c(C3CC3)c1=O)c1cc(ccc1F)Oc1cccc(C)c1-2]

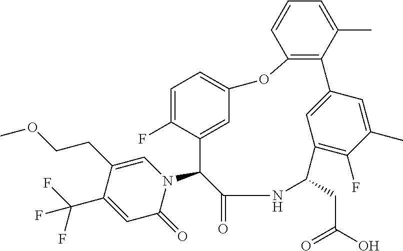

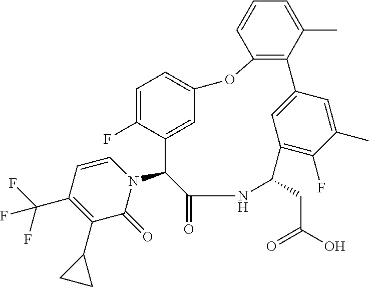 [SMILES: Cc1cc2cc(c1F)[C@H](CC(=O)O)NC(=O)[C@@H](n1ccc(C(F)(F)F)c(C3CC3)c1=O)c1cc(ccc1F)Oc1cccc(C)c1-2]

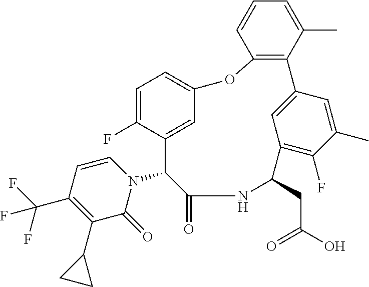 [SMILES: Cc1cc2cc(c1F)[C@@H](CC(=O)O)NC(=O)[C@H](n1ccc(C(F)(F)F)c(C3CC3)c1=O)c1cc(ccc1F)Oc1cccc(C)c1-2]

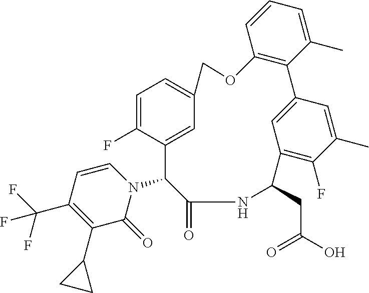 [SMILES: Cc1cc2cc(c1F)[C@@H](CC(=O)O)NC(=O)[C@H](n1ccc(C(F)(F)F)c(C3CC3)c1=O)c1cc(ccc1F)COc1cccc(C)c1-2]

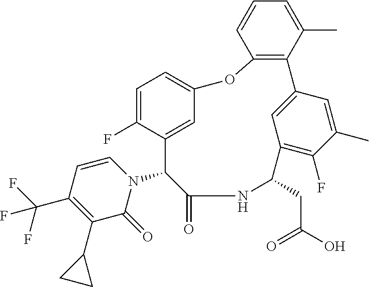 [SMILES: Cc1cc2cc(c1F)[C@H](CC(=O)O)NC(=O)[C@H](n1ccc(C(F)(F)F)c(C3CC3)c1=O)c1cc(ccc1F)Oc1cccc(C)c1-2]

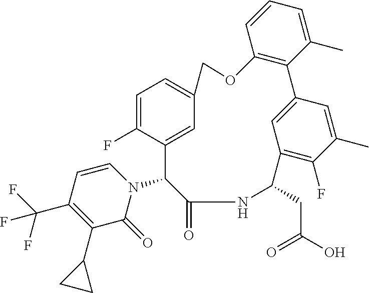 [SMILES: Cc1cc2cc(c1F)[C@H](CC(=O)O)NC(=O)[C@H](n1ccc(C(F)(F)F)c(C3CC3)c1=O)c1cc(ccc1F)COc1cccc(C)c1-2]

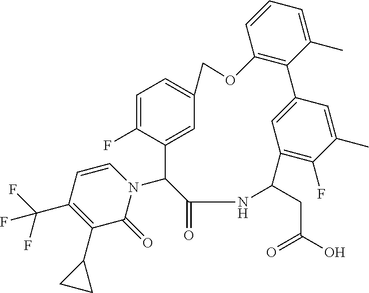 [SMILES: Cc1cc2cc(c1F)C(CC(=O)O)NC(=O)C(n1ccc(C(F)(F)F)c(C3CC3)c1=O)c1cc(ccc1F)COc1cccc(C)c1-2]

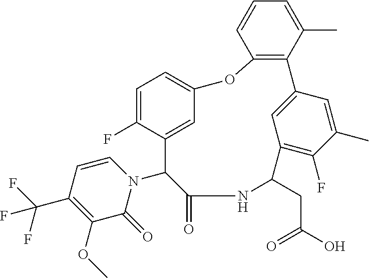 [SMILES: COc1c(C(F)(F)F)ccn(C2C(=O)NC(CC(=O)O)c3cc(cc(C)c3F)-c3c(C)cccc3Oc3ccc(F)c2c3)c1=O]

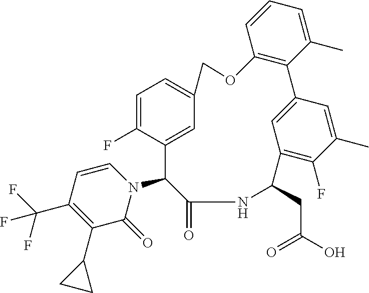 [SMILES: Cc1cc2cc(c1F)[C@@H](CC(=O)O)NC(=O)[C@@H](n1ccc(C(F)(F)F)c(C3CC3)c1=O)c1cc(ccc1F)COc1cccc(C)c1-2]

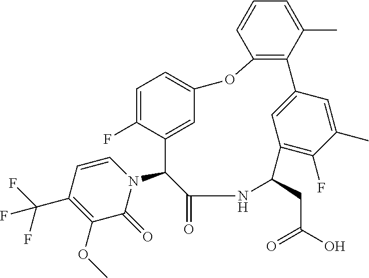 [SMILES: COc1c(C(F)(F)F)ccn([C@@H]2C(=O)N[C@H](CC(=O)O)c3cc(cc(C)c3F)-c3c(C)cccc3Oc3ccc(F)c2c3)c1=O]

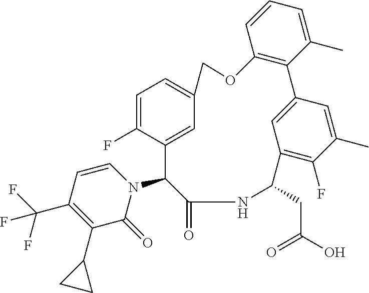 [SMILES: Cc1cc2cc(c1F)[C@H](CC(=O)O)NC(=O)[C@@H](n1ccc(C(F)(F)F)c(C3CC3)c1=O)c1cc(ccc1F)COc1cccc(C)c1-2]

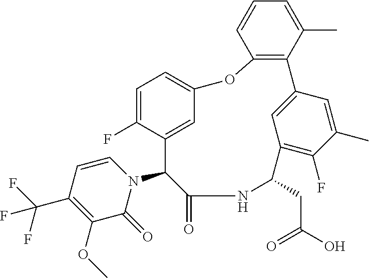 [SMILES: COc1c(C(F)(F)F)ccn([C@@H]2C(=O)N[C@@H](CC(=O)O)c3cc(cc(C)c3F)-c3c(C)cccc3Oc3ccc(F)c2c3)c1=O]

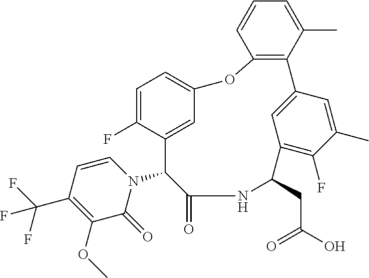 [SMILES: COc1c(C(F)(F)F)ccn([C@H]2C(=O)N[C@H](CC(=O)O)c3cc(cc(C)c3F)-c3c(C)cccc3Oc3ccc(F)c2c3)c1=O]

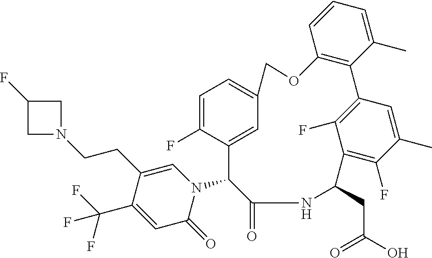 [SMILES: Cc1cc2c(F)c(c1F)[C@@H](CC(=O)O)NC(=O)[C@H](n1cc(CCN3CC(F)C3)c(C(F)(F)F)cc1=O)c1cc(ccc1F)COc1cccc(C)c1-2]

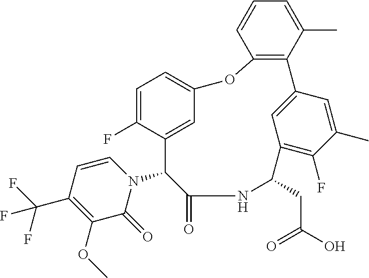 [SMILES: COc1c(C(F)(F)F)ccn([C@H]2C(=O)N[C@@H](CC(=O)O)c3cc(cc(C)c3F)-c3c(C)cccc3Oc3ccc(F)c2c3)c1=O]

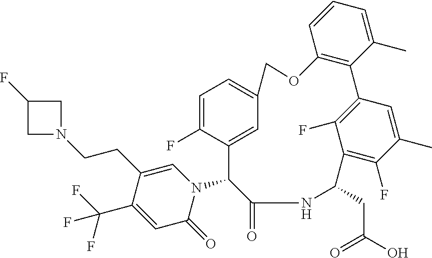 [SMILES: Cc1cc2c(F)c(c1F)[C@H](CC(=O)O)NC(=O)[C@H](n1cc(CCN3CC(F)C3)c(C(F)(F)F)cc1=O)c1cc(ccc1F)COc1cccc(C)c1-2]

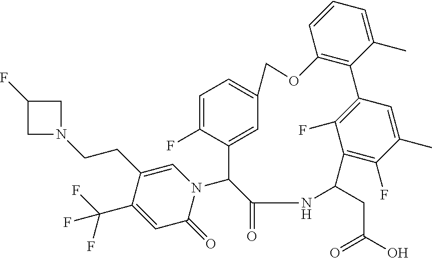 [SMILES: Cc1cc2c(F)c(c1F)C(CC(=O)O)NC(=O)C(n1cc(CCN3CC(F)C3)c(C(F)(F)F)cc1=O)c1cc(ccc1F)COc1cccc(C)c1-2]

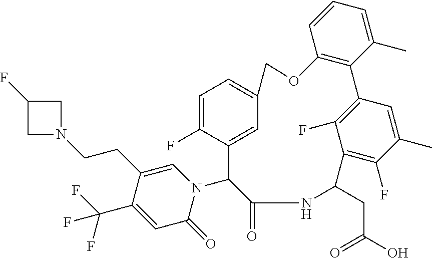 [SMILES: Cc1cc2c(F)c(c1F)C(CC(=O)O)NC(=O)C(n1cc(CCN3CC(F)C3)c(C(F)(F)F)cc1=O)c1cc(ccc1F)COc1cccc(C)c1-2]

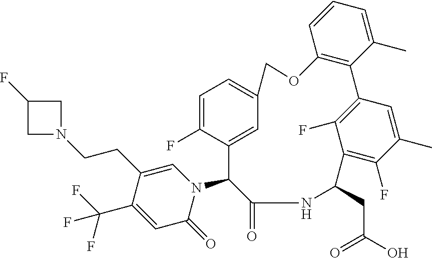 [SMILES: Cc1cc2c(F)c(c1F)[C@@H](CC(=O)O)NC(=O)[C@@H](n1cc(CCN3CC(F)C3)c(C(F)(F)F)cc1=O)c1cc(ccc1F)COc1cccc(C)c1-2]

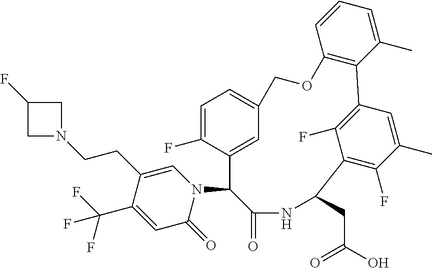 [SMILES: Cc1cc2c(F)c(c1F)[C@@H](CC(=O)O)NC(=O)[C@@H](n1cc(CCN3CC(F)C3)c(C(F)(F)F)cc1=O)c1cc(ccc1F)COc1cccc(C)c1-2]

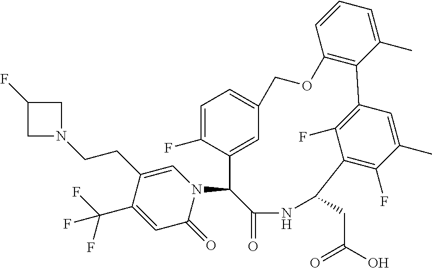 [SMILES: Cc1cc2c(F)c(c1F)[C@H](CC(=O)O)NC(=O)[C@@H](n1cc(CCN3CC(F)C3)c(C(F)(F)F)cc1=O)c1cc(ccc1F)COc1cccc(C)c1-2]

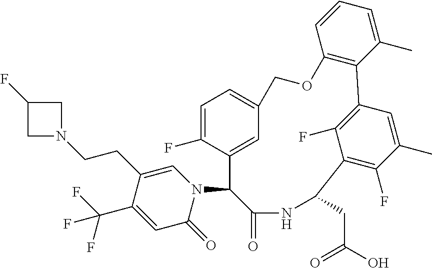 [SMILES: Cc1cc2c(F)c(c1F)[C@H](CC(=O)O)NC(=O)[C@@H](n1cc(CCN3CC(F)C3)c(C(F)(F)F)cc1=O)c1cc(ccc1F)COc1cccc(C)c1-2]

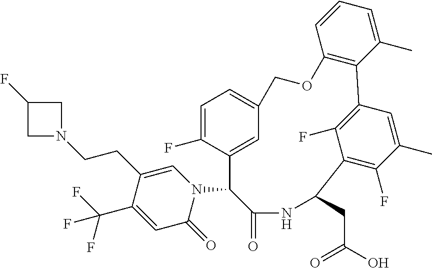 [SMILES: Cc1cc2c(F)c(c1F)[C@@H](CC(=O)O)NC(=O)[C@H](n1cc(CCN3CC(F)C3)c(C(F)(F)F)cc1=O)c1cc(ccc1F)COc1cccc(C)c1-2]

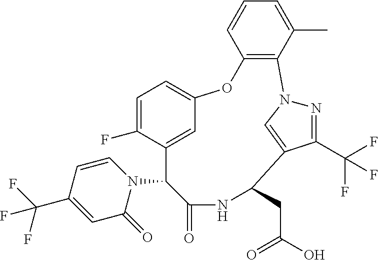 [SMILES: Cc1cccc2c1-n1cc(c(C(F)(F)F)n1)[C@@H](CC(=O)O)NC(=O)[C@H](n1ccc(C(F)(F)F)cc1=O)c1cc(ccc1F)O2]

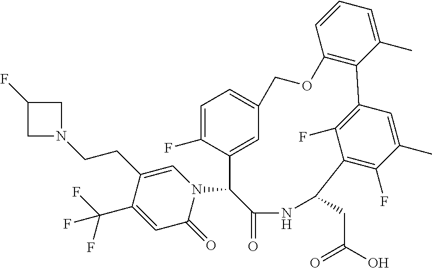 [SMILES: Cc1cc2c(F)c(c1F)[C@H](CC(=O)O)NC(=O)[C@H](n1cc(CCN3CC(F)C3)c(C(F)(F)F)cc1=O)c1cc(ccc1F)COc1cccc(C)c1-2]

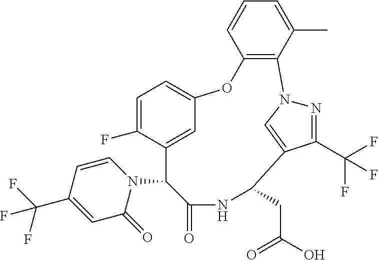 [SMILES: Cc1cccc2c1-n1cc(c(C(F)(F)F)n1)[C@H](CC(=O)O)NC(=O)[C@H](n1ccc(C(F)(F)F)cc1=O)c1cc(ccc1F)O2]

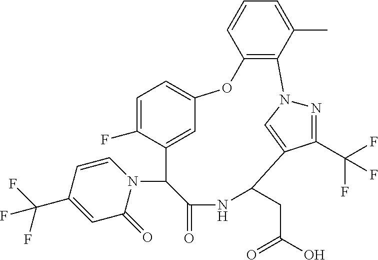 [SMILES: Cc1cccc2c1-n1cc(c(C(F)(F)F)n1)C(CC(=O)O)NC(=O)C(n1ccc(C(F)(F)F)cc1=O)c1cc(ccc1F)O2]

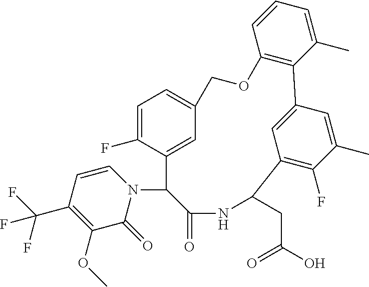 [SMILES: COc1c(C(F)(F)F)ccn(C2C(=O)NC(CC(=O)O)c3cc(cc(C)c3F)-c3c(C)cccc3OCc3ccc(F)c2c3)c1=O]

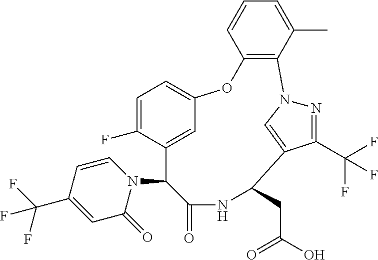 [SMILES: Cc1cccc2c1-n1cc(c(C(F)(F)F)n1)[C@@H](CC(=O)O)NC(=O)[C@@H](n1ccc(C(F)(F)F)cc1=O)c1cc(ccc1F)O2]

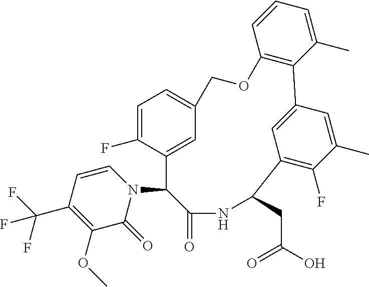 [SMILES: COc1c(C(F)(F)F)ccn([C@@H]2C(=O)N[C@H](CC(=O)O)c3cc(cc(C)c3F)-c3c(C)cccc3OCc3ccc(F)c2c3)c1=O]

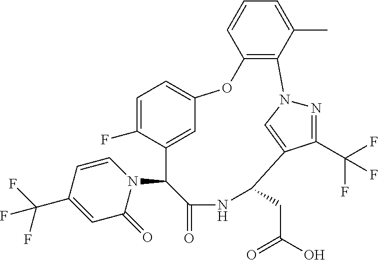 [SMILES: Cc1cccc2c1-n1cc(c(C(F)(F)F)n1)[C@H](CC(=O)O)NC(=O)[C@@H](n1ccc(C(F)(F)F)cc1=O)c1cc(ccc1F)O2]

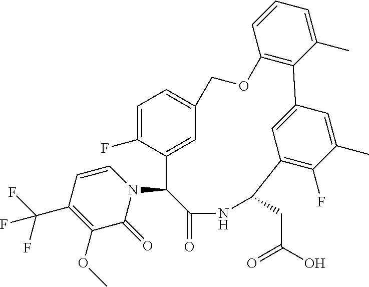 [SMILES: COc1c(C(F)(F)F)ccn([C@@H]2C(=O)N[C@@H](CC(=O)O)c3cc(cc(C)c3F)-c3c(C)cccc3OCc3ccc(F)c2c3)c1=O]

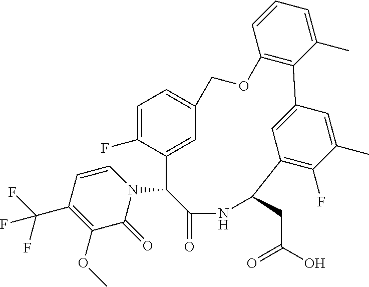 [SMILES: COc1c(C(F)(F)F)ccn([C@H]2C(=O)N[C@H](CC(=O)O)c3cc(cc(C)c3F)-c3c(C)cccc3OCc3ccc(F)c2c3)c1=O]

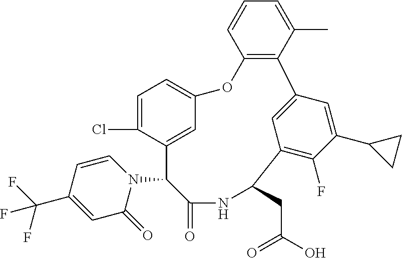 [SMILES: Cc1cccc2c1-c1cc(C3CC3)c(F)c(c1)[C@@H](CC(=O)O)NC(=O)[C@H](n1ccc(C(F)(F)F)cc1=O)c1cc(ccc1Cl)O2]

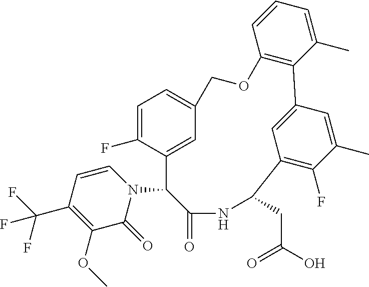 [SMILES: COc1c(C(F)(F)F)ccn([C@H]2C(=O)N[C@@H](CC(=O)O)c3cc(cc(C)c3F)-c3c(C)cccc3OCc3ccc(F)c2c3)c1=O]

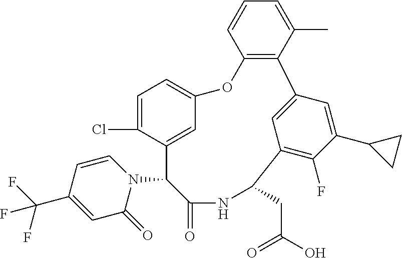 [SMILES: Cc1cccc2c1-c1cc(C3CC3)c(F)c(c1)[C@H](CC(=O)O)NC(=O)[C@H](n1ccc(C(F)(F)F)cc1=O)c1cc(ccc1Cl)O2]

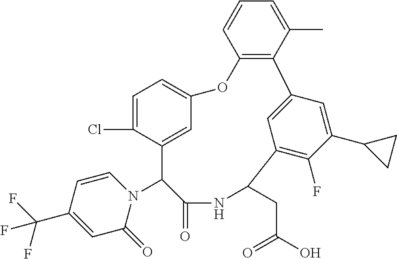 [SMILES: Cc1cccc2c1-c1cc(C3CC3)c(F)c(c1)C(CC(=O)O)NC(=O)C(n1ccc(C(F)(F)F)cc1=O)c1cc(ccc1Cl)O2]

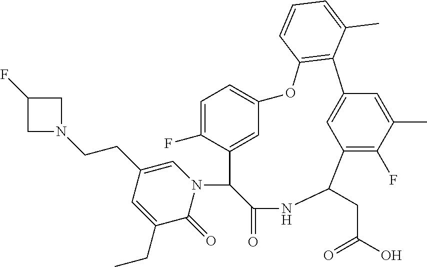 [SMILES: CCc1cc(CCN2CC(F)C2)cn(C2C(=O)NC(CC(=O)O)c3cc(cc(C)c3F)-c3c(C)cccc3Oc3ccc(F)c2c3)c1=O]

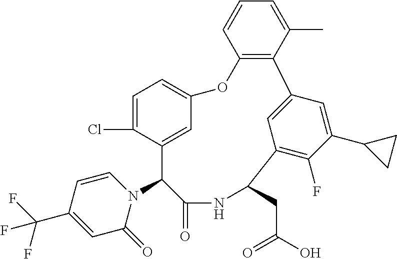 [SMILES: Cc1cccc2c1-c1cc(C3CC3)c(F)c(c1)[C@@H](CC(=O)O)NC(=O)[C@@H](n1ccc(C(F)(F)F)cc1=O)c1cc(ccc1Cl)O2]

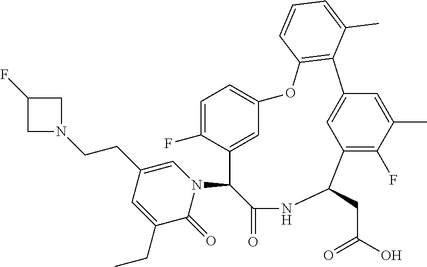 [SMILES: CCc1cc(CCN2CC(F)C2)cn([C@@H]2C(=O)N[C@H](CC(=O)O)c3cc(cc(C)c3F)-c3c(C)cccc3Oc3ccc(F)c2c3)c1=O]

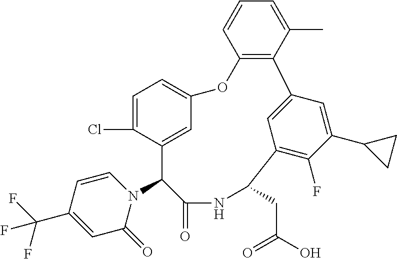 [SMILES: Cc1cccc2c1-c1cc(C3CC3)c(F)c(c1)[C@H](CC(=O)O)NC(=O)[C@@H](n1ccc(C(F)(F)F)cc1=O)c1cc(ccc1Cl)O2]

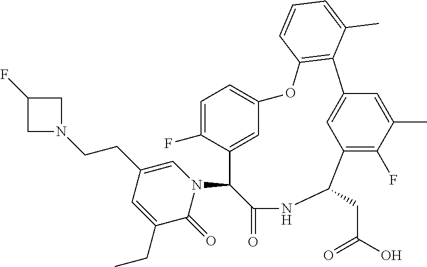 [SMILES: CCc1cc(CCN2CC(F)C2)cn([C@@H]2C(=O)N[C@@H](CC(=O)O)c3cc(cc(C)c3F)-c3c(C)cccc3Oc3ccc(F)c2c3)c1=O]

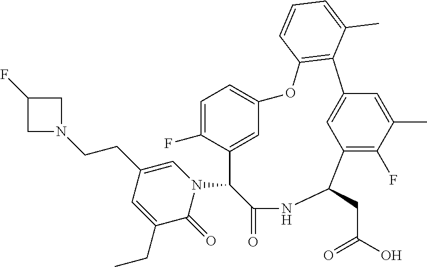 [SMILES: CCc1cc(CCN2CC(F)C2)cn([C@H]2C(=O)N[C@H](CC(=O)O)c3cc(cc(C)c3F)-c3c(C)cccc3Oc3ccc(F)c2c3)c1=O]

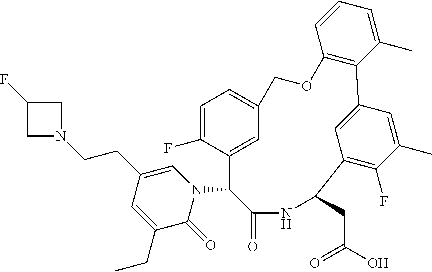 [SMILES: CCc1cc(CCN2CC(F)C2)cn([C@H]2C(=O)N[C@H](CC(=O)O)c3cc(cc(C)c3F)-c3c(C)cccc3OCc3ccc(F)c2c3)c1=O]

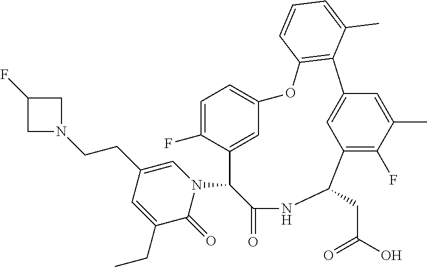 [SMILES: CCc1cc(CCN2CC(F)C2)cn([C@H]2C(=O)N[C@@H](CC(=O)O)c3cc(cc(C)c3F)-c3c(C)cccc3Oc3ccc(F)c2c3)c1=O]

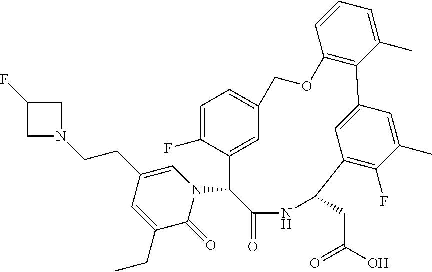 [SMILES: CCc1cc(CCN2CC(F)C2)cn([C@H]2C(=O)N[C@@H](CC(=O)O)c3cc(cc(C)c3F)-c3c(C)cccc3OCc3ccc(F)c2c3)c1=O]

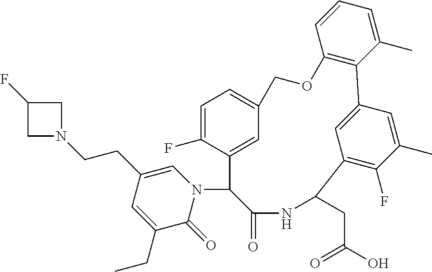 [SMILES: CCc1cc(CCN2CC(F)C2)cn(C2C(=O)NC(CC(=O)O)c3cc(cc(C)c3F)-c3c(C)cccc3OCc3ccc(F)c2c3)c1=O]

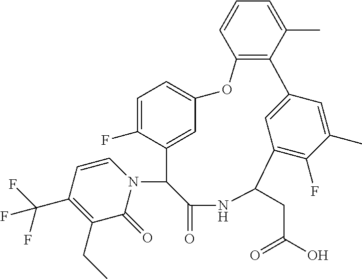 [SMILES: CCc1c(C(F)(F)F)ccn(C2C(=O)NC(CC(=O)O)c3cc(cc(C)c3F)-c3c(C)cccc3Oc3ccc(F)c2c3)c1=O]

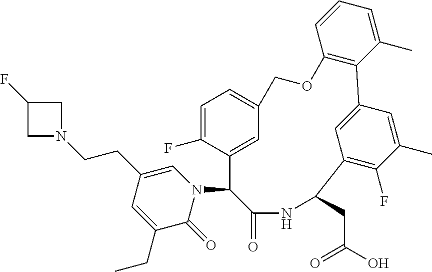 [SMILES: CCc1cc(CCN2CC(F)C2)cn([C@@H]2C(=O)N[C@H](CC(=O)O)c3cc(cc(C)c3F)-c3c(C)cccc3OCc3ccc(F)c2c3)c1=O]

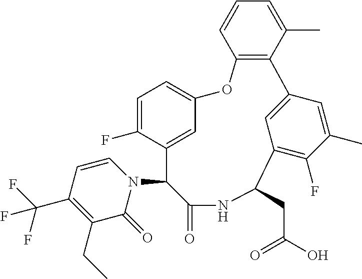 [SMILES: CCc1c(C(F)(F)F)ccn([C@@H]2C(=O)N[C@H](CC(=O)O)c3cc(cc(C)c3F)-c3c(C)cccc3Oc3ccc(F)c2c3)c1=O]

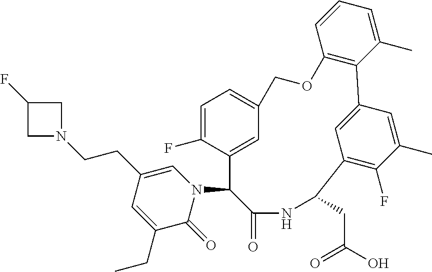 [SMILES: CCc1cc(CCN2CC(F)C2)cn([C@@H]2C(=O)N[C@@H](CC(=O)O)c3cc(cc(C)c3F)-c3c(C)cccc3OCc3ccc(F)c2c3)c1=O]

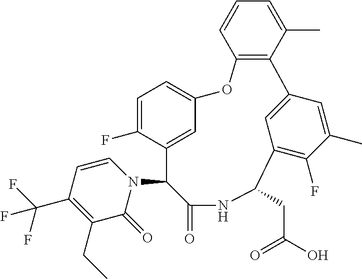 [SMILES: CCc1c(C(F)(F)F)ccn([C@@H]2C(=O)N[C@@H](CC(=O)O)c3cc(cc(C)c3F)-c3c(C)cccc3Oc3ccc(F)c2c3)c1=O]

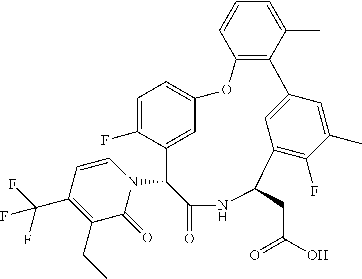 [SMILES: CCc1c(C(F)(F)F)ccn([C@H]2C(=O)N[C@H](CC(=O)O)c3cc(cc(C)c3F)-c3c(C)cccc3Oc3ccc(F)c2c3)c1=O]

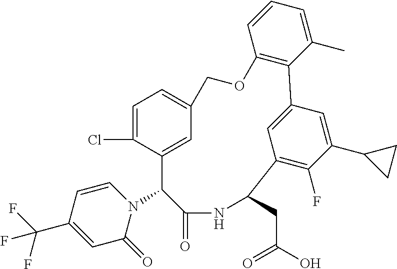 [SMILES: Cc1cccc2c1-c1cc(C3CC3)c(F)c(c1)[C@@H](CC(=O)O)NC(=O)[C@H](n1ccc(C(F)(F)F)cc1=O)c1cc(ccc1Cl)CO2]

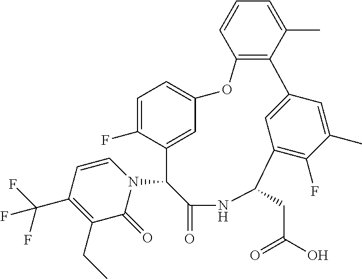 [SMILES: CCc1c(C(F)(F)F)ccn([C@H]2C(=O)N[C@@H](CC(=O)O)c3cc(cc(C)c3F)-c3c(C)cccc3Oc3ccc(F)c2c3)c1=O]

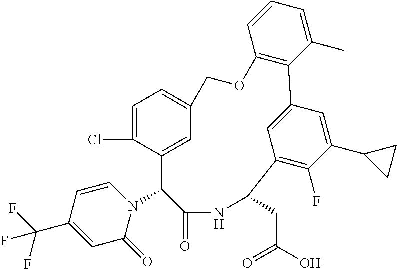 [SMILES: Cc1cccc2c1-c1cc(C3CC3)c(F)c(c1)[C@H](CC(=O)O)NC(=O)[C@H](n1ccc(C(F)(F)F)cc1=O)c1cc(ccc1Cl)CO2]

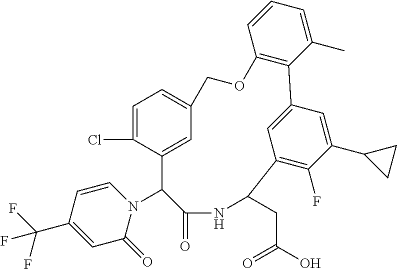 [SMILES: Cc1cccc2c1-c1cc(C3CC3)c(F)c(c1)C(CC(=O)O)NC(=O)C(n1ccc(C(F)(F)F)cc1=O)c1cc(ccc1Cl)CO2]

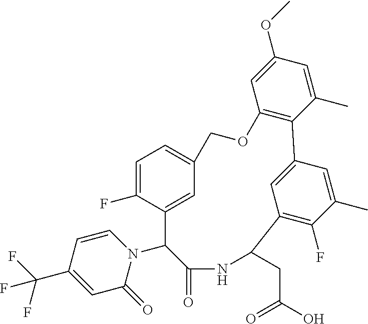 [SMILES: COc1cc(C)c2c(c1)OCc1ccc(F)c(c1)C(n1ccc(C(F)(F)F)cc1=O)C(=O)NC(CC(=O)O)c1cc-2cc(C)c1F]

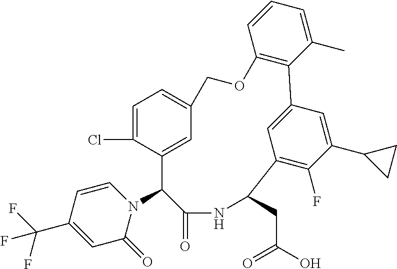 [SMILES: Cc1cccc2c1-c1cc(C3CC3)c(F)c(c1)[C@@H](CC(=O)O)NC(=O)[C@@H](n1ccc(C(F)(F)F)cc1=O)c1cc(ccc1Cl)CO2]

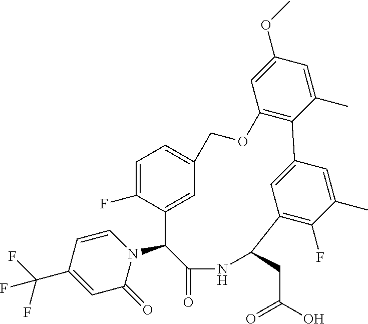 [SMILES: COc1cc(C)c2c(c1)OCc1ccc(F)c(c1)[C@H](n1ccc(C(F)(F)F)cc1=O)C(=O)N[C@H](CC(=O)O)c1cc-2cc(C)c1F]

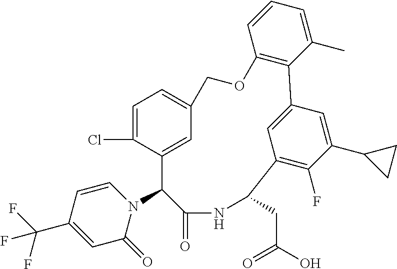 [SMILES: Cc1cccc2c1-c1cc(C3CC3)c(F)c(c1)[C@H](CC(=O)O)NC(=O)[C@@H](n1ccc(C(F)(F)F)cc1=O)c1cc(ccc1Cl)CO2]

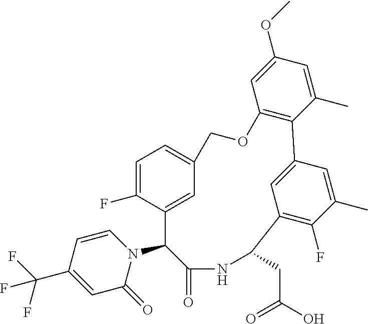 [SMILES: COc1cc(C)c2c(c1)OCc1ccc(F)c(c1)[C@H](n1ccc(C(F)(F)F)cc1=O)C(=O)N[C@@H](CC(=O)O)c1cc-2cc(C)c1F]

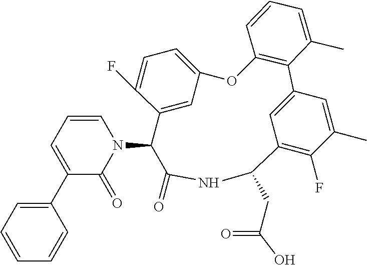 [SMILES: Cc1cc2cc(c1F)[C@H](CC(=O)O)NC(=O)[C@@H](n1cccc(-c3ccccc3)c1=O)c1cc(ccc1F)Oc1cccc(C)c1-2]

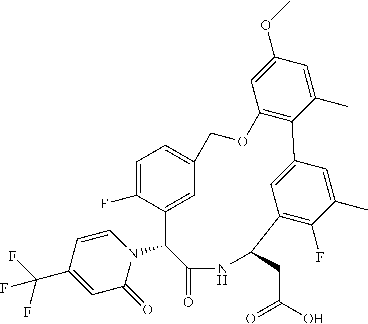 [SMILES: COc1cc(C)c2c(c1)OCc1ccc(F)c(c1)[C@@H](n1ccc(C(F)(F)F)cc1=O)C(=O)N[C@H](CC(=O)O)c1cc-2cc(C)c1F]

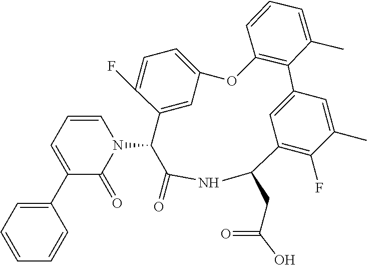 [SMILES: Cc1cc2cc(c1F)[C@@H](CC(=O)O)NC(=O)[C@H](n1cccc(-c3ccccc3)c1=O)c1cc(ccc1F)Oc1cccc(C)c1-2]

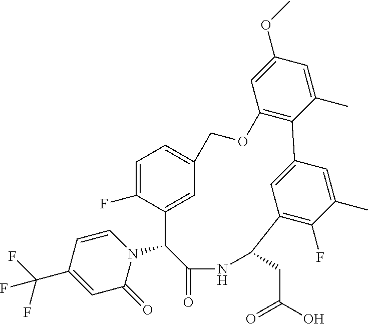 [SMILES: COc1cc(C)c2c(c1)OCc1ccc(F)c(c1)[C@@H](n1ccc(C(F)(F)F)cc1=O)C(=O)N[C@@H](CC(=O)O)c1cc-2cc(C)c1F]

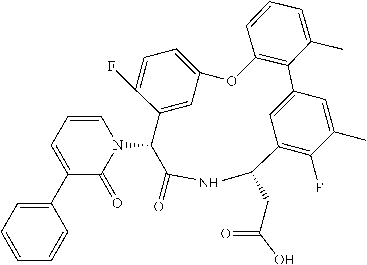 [SMILES: Cc1cc2cc(c1F)[C@H](CC(=O)O)NC(=O)[C@H](n1cccc(-c3ccccc3)c1=O)c1cc(ccc1F)Oc1cccc(C)c1-2]

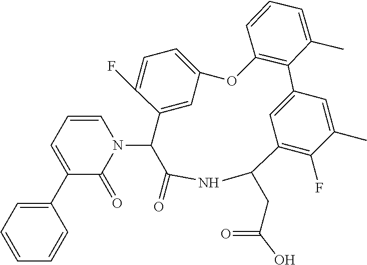 [SMILES: Cc1cc2cc(c1F)C(CC(=O)O)NC(=O)C(n1cccc(-c3ccccc3)c1=O)c1cc(ccc1F)Oc1cccc(C)c1-2]

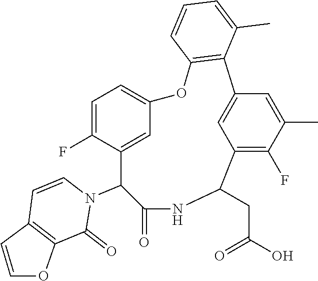 [SMILES: Cc1cc2cc(c1F)C(CC(=O)O)NC(=O)C(n1ccc3ccoc3c1=O)c1cc(ccc1F)Oc1cccc(C)c1-2]

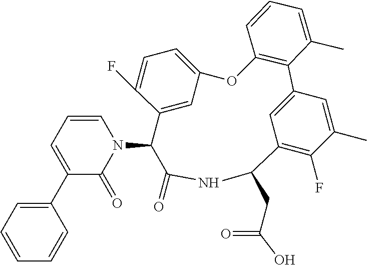 [SMILES: Cc1cc2cc(c1F)[C@@H](CC(=O)O)NC(=O)[C@@H](n1cccc(-c3ccccc3)c1=O)c1cc(ccc1F)Oc1cccc(C)c1-2]

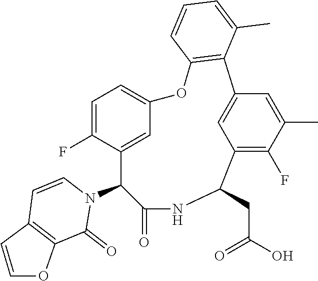 [SMILES: Cc1cc2cc(c1F)[C@@H](CC(=O)O)NC(=O)[C@@H](n1ccc3ccoc3c1=O)c1cc(ccc1F)Oc1cccc(C)c1-2]

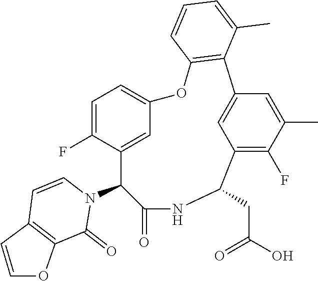 [SMILES: Cc1cc2cc(c1F)[C@H](CC(=O)O)NC(=O)[C@@H](n1ccc3ccoc3c1=O)c1cc(ccc1F)Oc1cccc(C)c1-2]

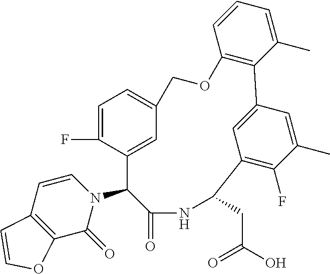 [SMILES: Cc1cc2cc(c1F)[C@H](CC(=O)O)NC(=O)[C@@H](n1ccc3ccoc3c1=O)c1cc(ccc1F)COc1cccc(C)c1-2]

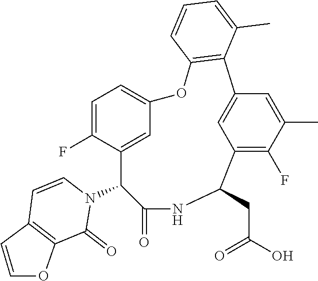 [SMILES: Cc1cc2cc(c1F)[C@@H](CC(=O)O)NC(=O)[C@H](n1ccc3ccoc3c1=O)c1cc(ccc1F)Oc1cccc(C)c1-2]

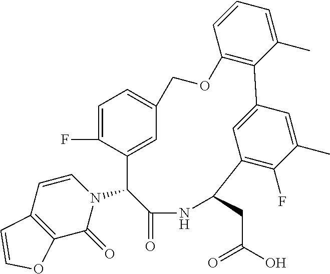 [SMILES: Cc1cc2cc(c1F)[C@@H](CC(=O)O)NC(=O)[C@H](n1ccc3ccoc3c1=O)c1cc(ccc1F)COc1cccc(C)c1-2]

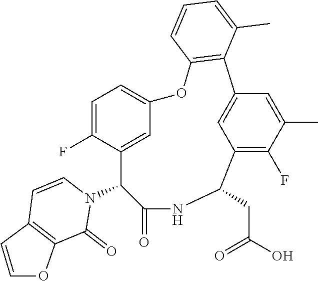 [SMILES: Cc1cc2cc(c1F)[C@H](CC(=O)O)NC(=O)[C@H](n1ccc3ccoc3c1=O)c1cc(ccc1F)Oc1cccc(C)c1-2]

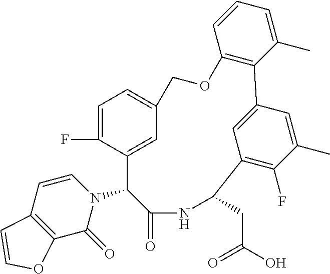 [SMILES: Cc1cc2cc(c1F)[C@H](CC(=O)O)NC(=O)[C@H](n1ccc3ccoc3c1=O)c1cc(ccc1F)COc1cccc(C)c1-2]

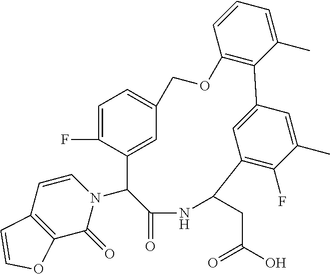 [SMILES: Cc1cc2cc(c1F)C(CC(=O)O)NC(=O)C(n1ccc3ccoc3c1=O)c1cc(ccc1F)COc1cccc(C)c1-2]

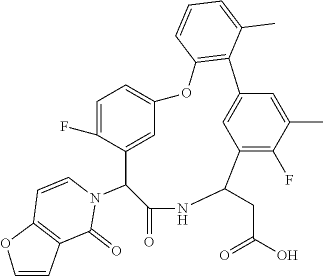 [SMILES: Cc1cc2cc(c1F)C(CC(=O)O)NC(=O)C(n1ccc3occc3c1=O)c1cc(ccc1F)Oc1cccc(C)c1-2]

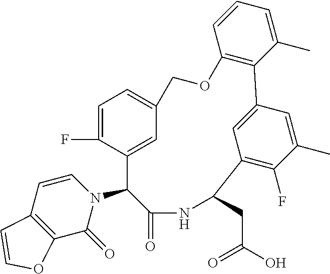 [SMILES: Cc1cc2cc(c1F)[C@@H](CC(=O)O)NC(=O)[C@@H](n1ccc3ccoc3c1=O)c1cc(ccc1F)COc1cccc(C)c1-2]

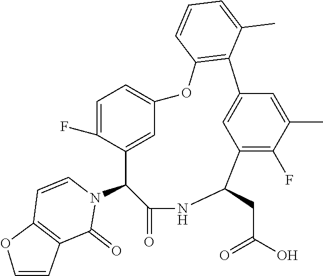 [SMILES: Cc1cc2cc(c1F)[C@@H](CC(=O)O)NC(=O)[C@@H](n1ccc3occc3c1=O)c1cc(ccc1F)Oc1cccc(C)c1-2]

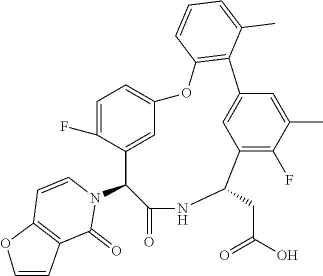 [SMILES: Cc1cc2cc(c1F)[C@H](CC(=O)O)NC(=O)[C@@H](n1ccc3occc3c1=O)c1cc(ccc1F)Oc1cccc(C)c1-2]

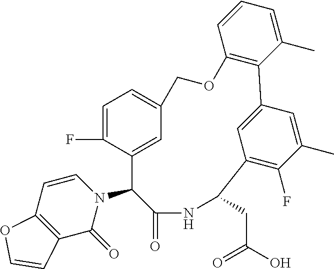 [SMILES: Cc1cc2cc(c1F)[C@H](CC(=O)O)NC(=O)[C@@H](n1ccc3occc3c1=O)c1cc(ccc1F)COc1cccc(C)c1-2]

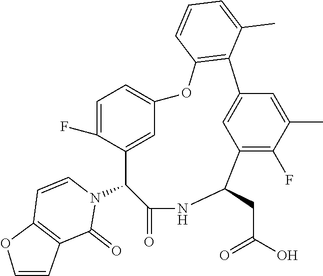 [SMILES: Cc1cc2cc(c1F)[C@@H](CC(=O)O)NC(=O)[C@H](n1ccc3occc3c1=O)c1cc(ccc1F)Oc1cccc(C)c1-2]

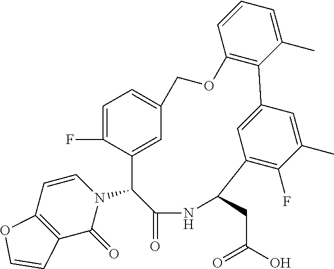 [SMILES: Cc1cc2cc(c1F)[C@@H](CC(=O)O)NC(=O)[C@H](n1ccc3occc3c1=O)c1cc(ccc1F)COc1cccc(C)c1-2]

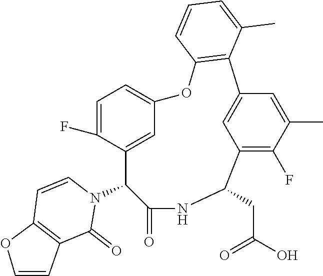 [SMILES: Cc1cc2cc(c1F)[C@H](CC(=O)O)NC(=O)[C@H](n1ccc3occc3c1=O)c1cc(ccc1F)Oc1cccc(C)c1-2]

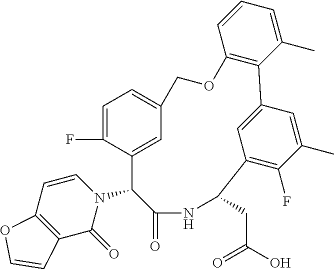 [SMILES: Cc1cc2cc(c1F)[C@H](CC(=O)O)NC(=O)[C@H](n1ccc3occc3c1=O)c1cc(ccc1F)COc1cccc(C)c1-2]

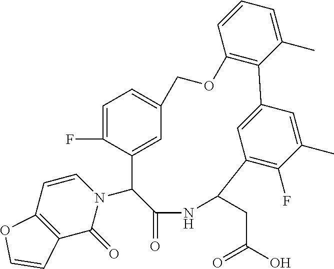 [SMILES: Cc1cc2cc(c1F)C(CC(=O)O)NC(=O)C(n1ccc3occc3c1=O)c1cc(ccc1F)COc1cccc(C)c1-2]

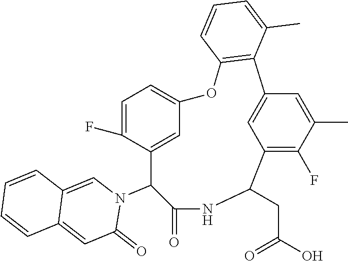 [SMILES: Cc1cc2cc(c1F)C(CC(=O)O)NC(=O)C(n1cc3ccccc3cc1=O)c1cc(ccc1F)Oc1cccc(C)c1-2]

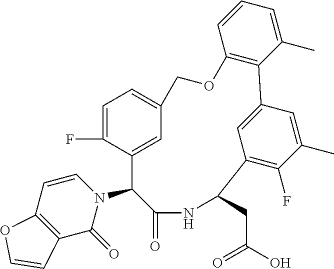 [SMILES: Cc1cc2cc(c1F)[C@@H](CC(=O)O)NC(=O)[C@@H](n1ccc3occc3c1=O)c1cc(ccc1F)COc1cccc(C)c1-2]

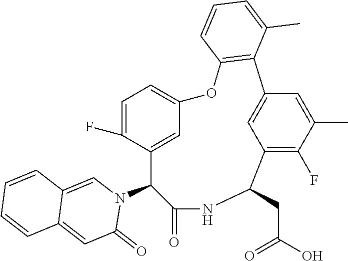 [SMILES: Cc1cc2cc(c1F)[C@@H](CC(=O)O)NC(=O)[C@@H](n1cc3ccccc3cc1=O)c1cc(ccc1F)Oc1cccc(C)c1-2]

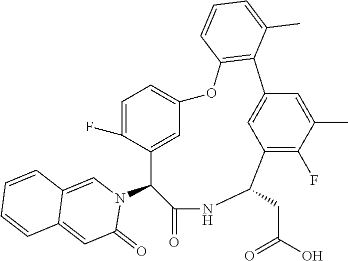 [SMILES: Cc1cc2cc(c1F)[C@H](CC(=O)O)NC(=O)[C@@H](n1cc3ccccc3cc1=O)c1cc(ccc1F)Oc1cccc(C)c1-2]

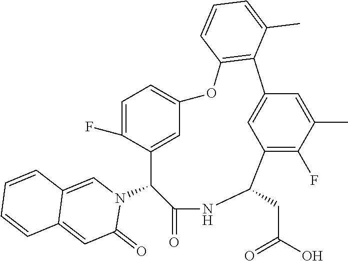 [SMILES: Cc1cc2cc(c1F)[C@H](CC(=O)O)NC(=O)[C@H](n1cc3ccccc3cc1=O)c1cc(ccc1F)Oc1cccc(C)c1-2]

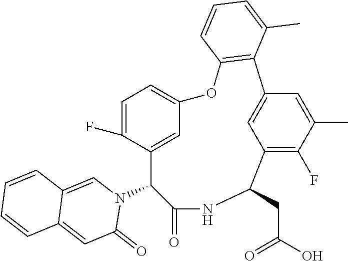 [SMILES: Cc1cc2cc(c1F)[C@@H](CC(=O)O)NC(=O)[C@H](n1cc3ccccc3cc1=O)c1cc(ccc1F)Oc1cccc(C)c1-2]

In some embodiments, the compound disclosed herein, or a pharmaceutically acceptable salt, solvate, or stereoisomer thereof, is selected from the group consisting of:

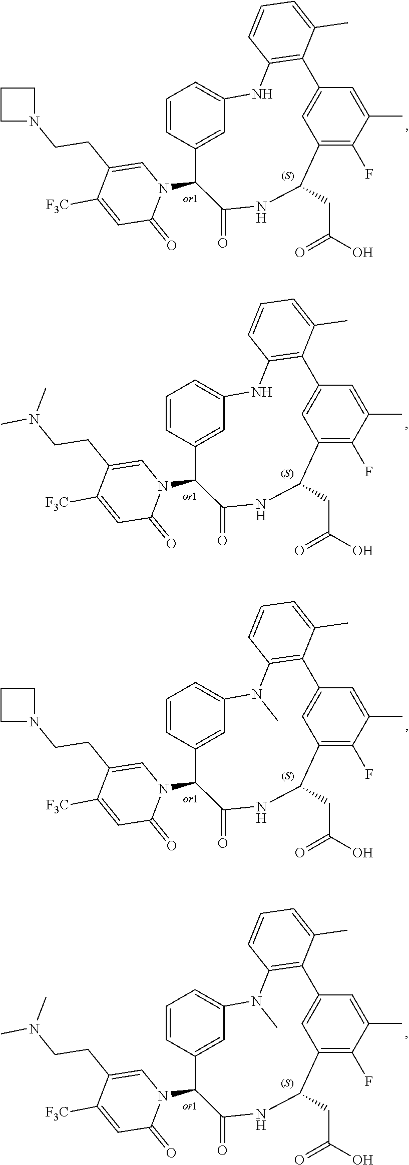

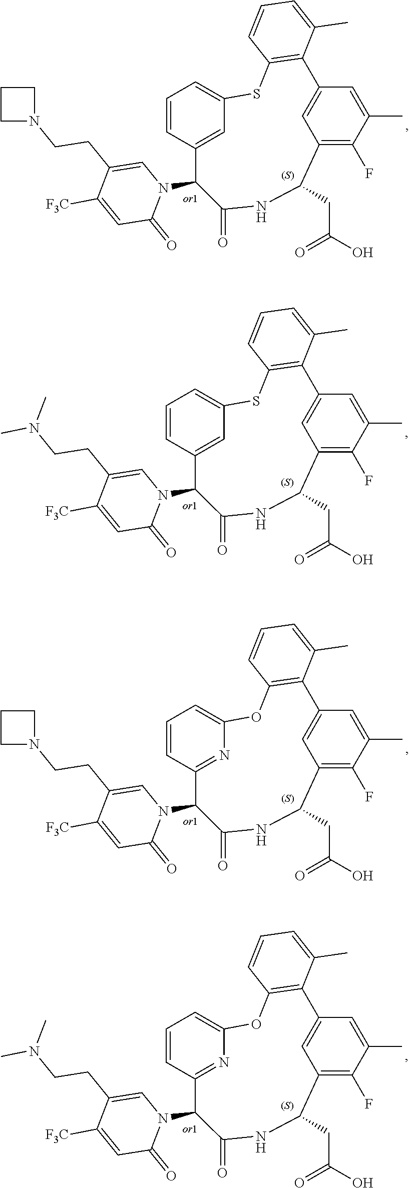

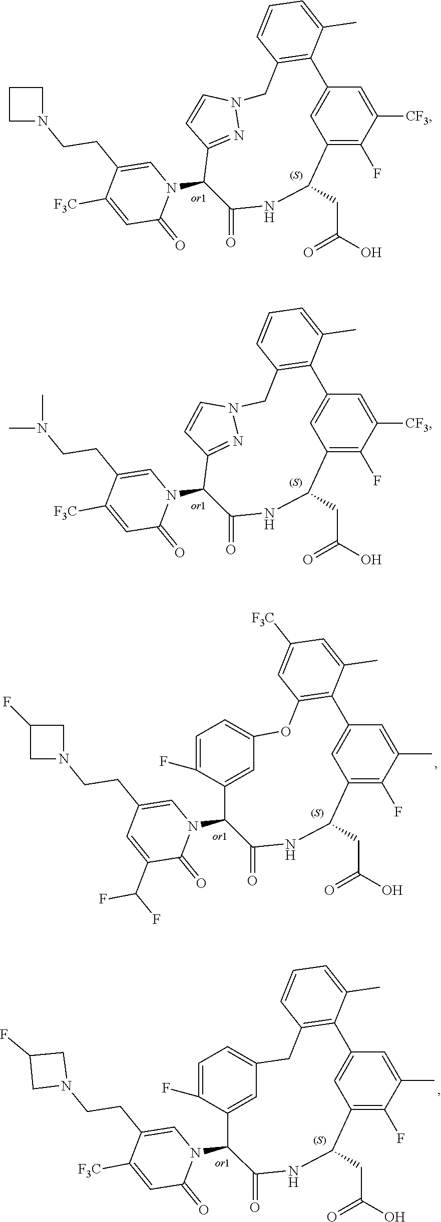

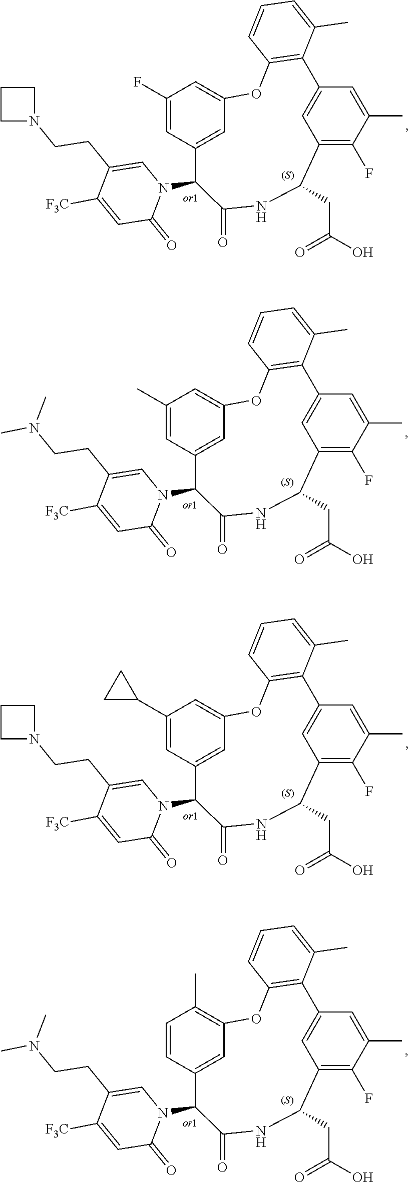

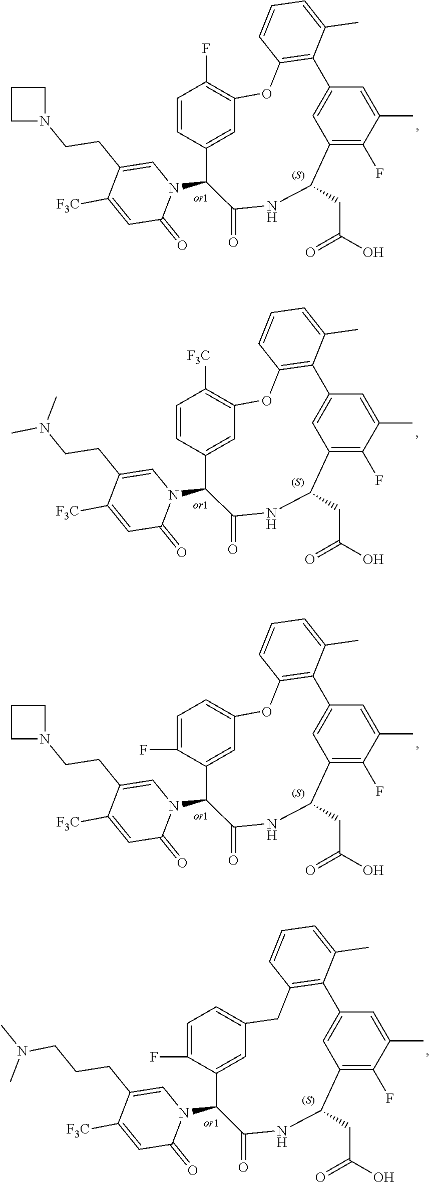

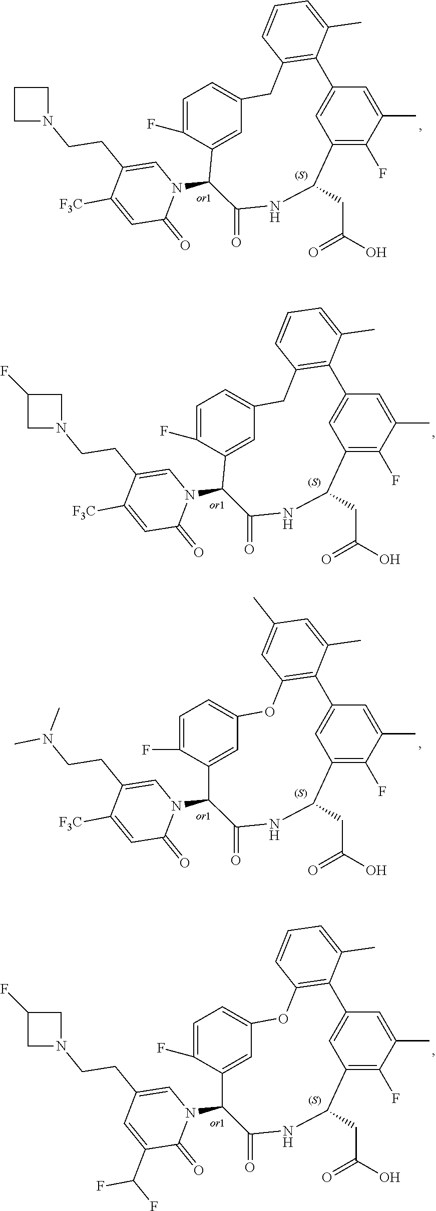

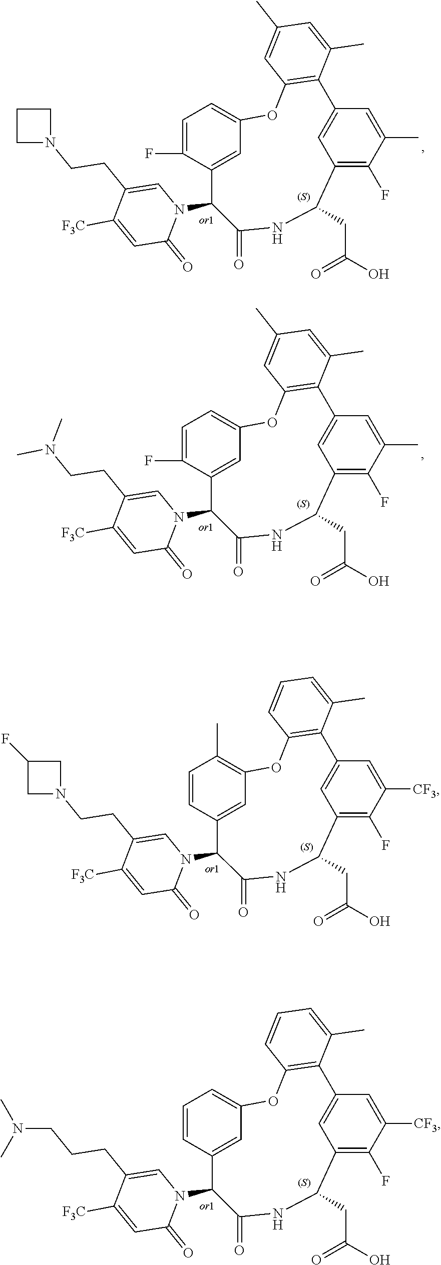

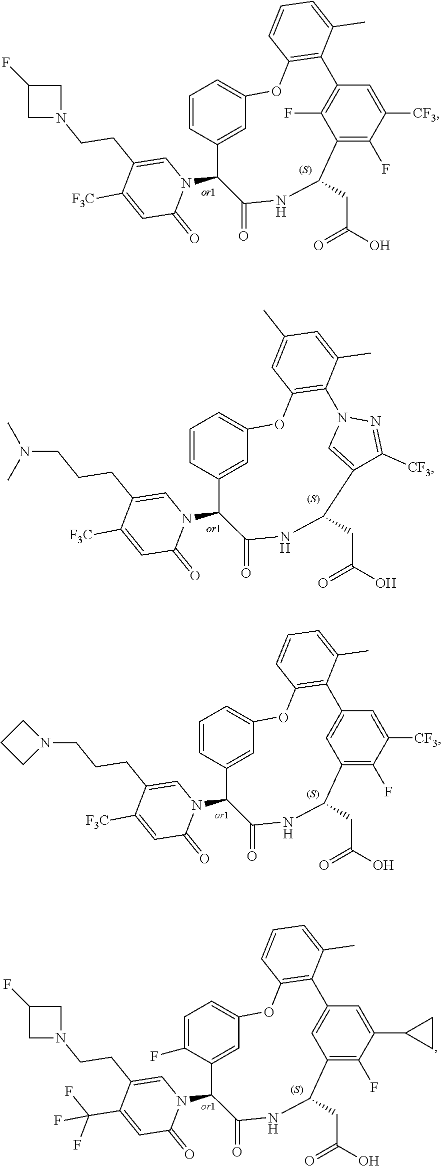

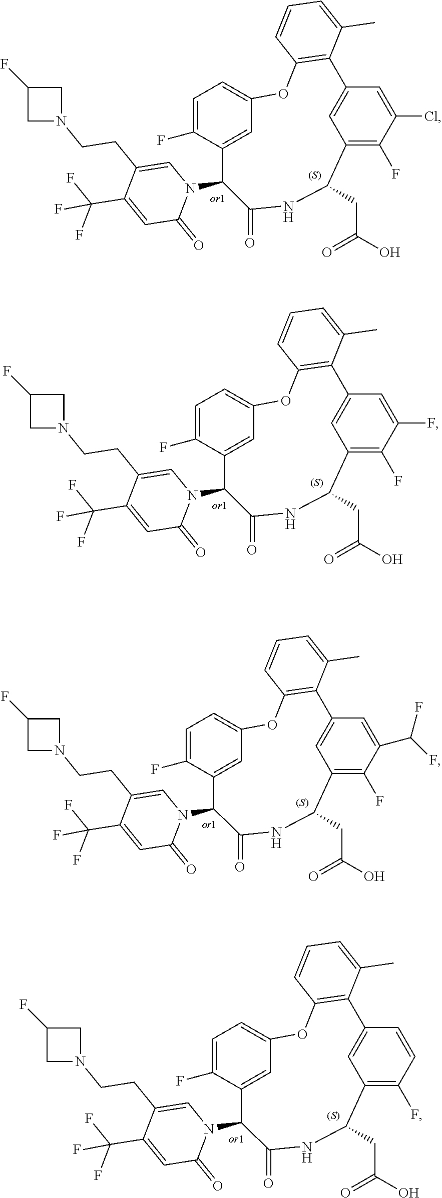

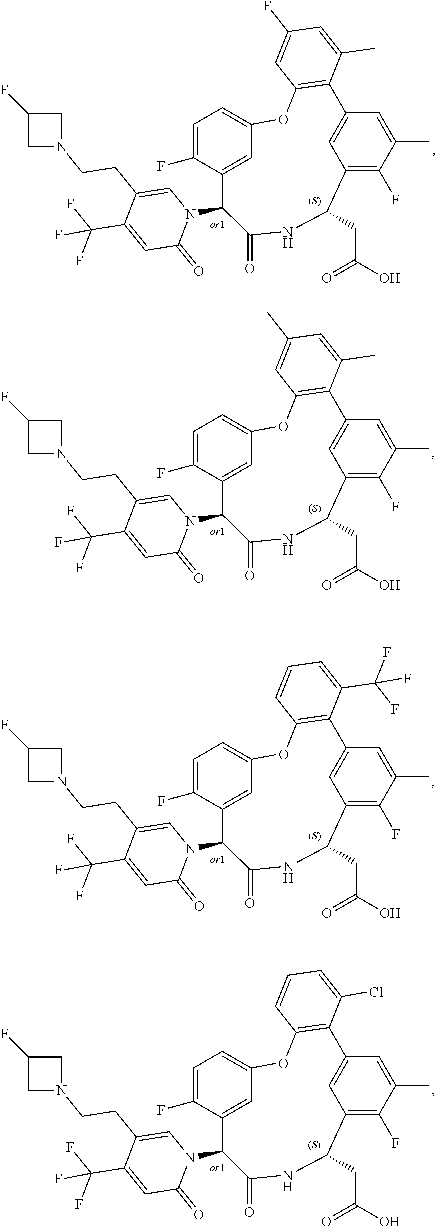

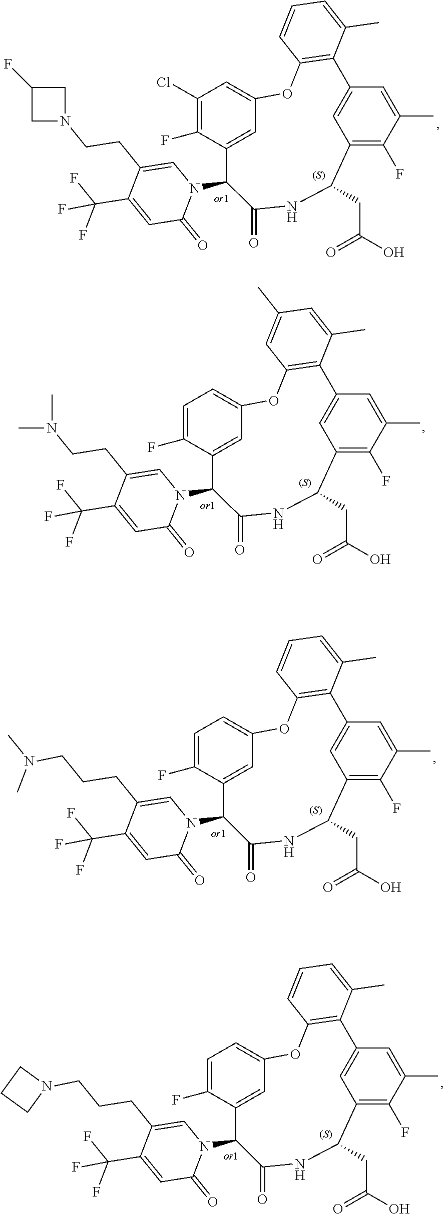

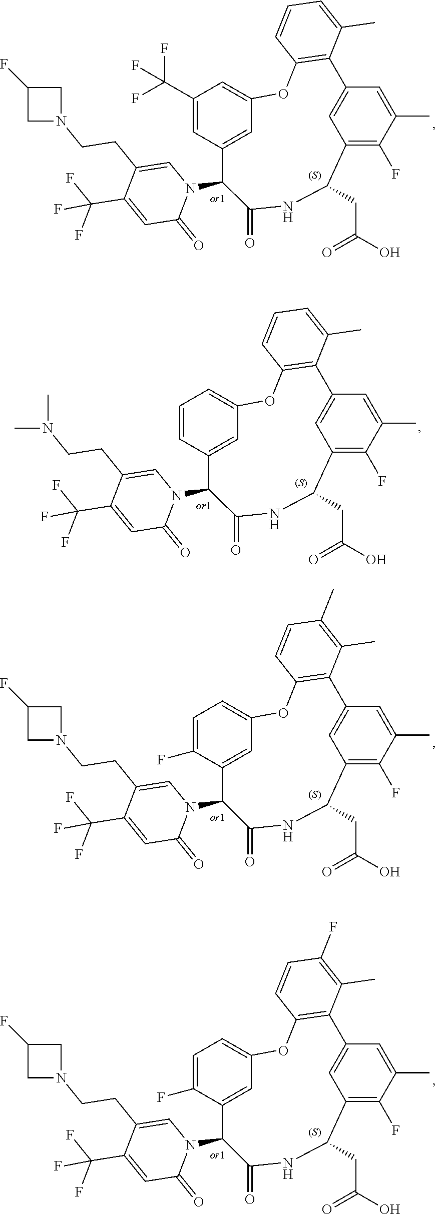

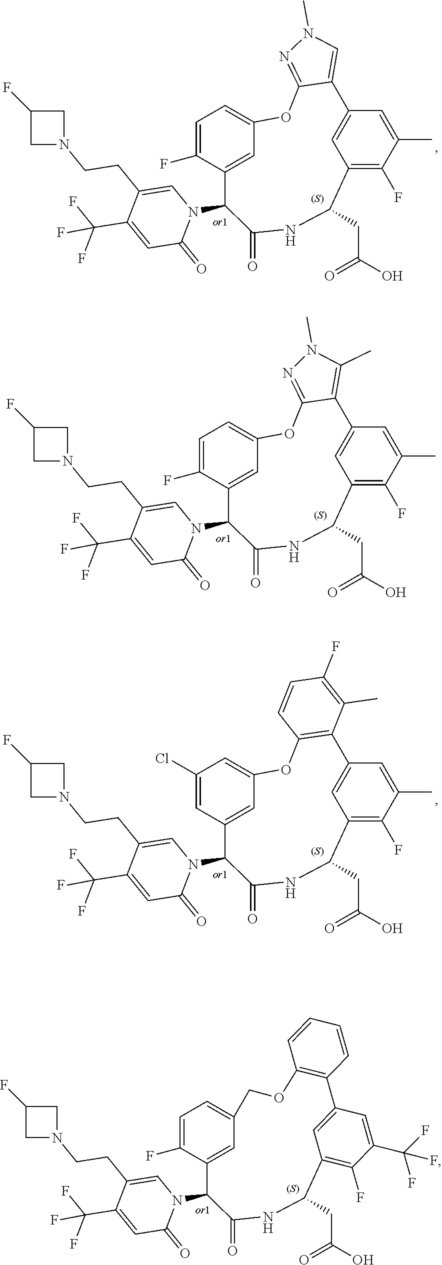

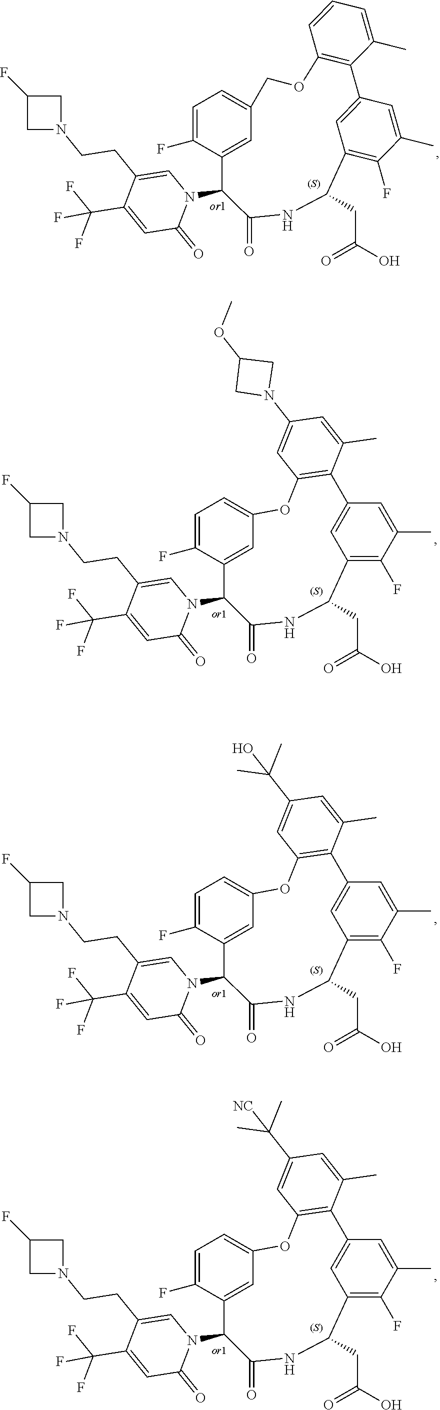

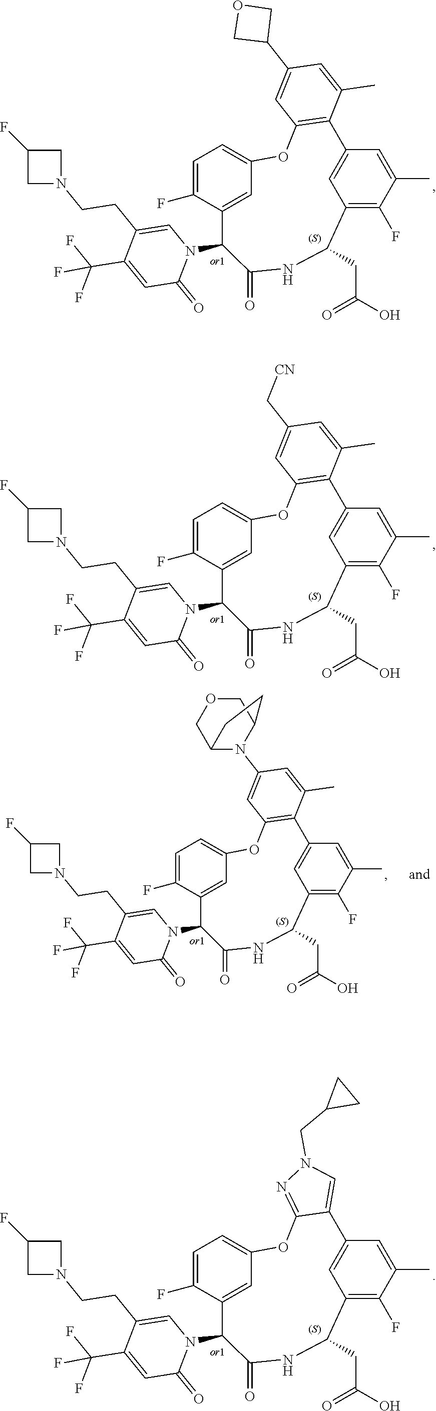

### Further Forms of Compounds Disclosed Herein

### Isomers Stereoisomers

In some embodiments, the compounds described herein exist as geometric isomers. In some embodiments, the compounds described herein possess one or more double bonds. The compounds presented herein include all cis, trans, syn, anti, entgegen (E), and zusammen (Z) isomers as well as the corresponding mixtures thereof. In some situations, the compounds described herein possess one or more chiral centers and each center exists in the R configuration, or S configuration. The compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as their individual stereoisomers by reacting a racemic mixture of the compound with an optically active resolving agent to form a pair of diastereoisomeric compounds, separating the diastereomers and recovering the optically pure enantiomers. In some embodiments, dissociable complexes are preferred. In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, e.g., by separation/resolution techniques based upon differences in solubility. In some embodiments, the optically pure enantiomer is then recovered, along with the resolving agent, by any practical means that would not result in racemization.

### Labeled Compounds

In some embodiments, the compounds described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions. Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. Examples of isotopes that can be incorporated into compounds disclosed herein include isotopes of hydrogen, carbon, nitrogen, oxygen, phosphorous, sulfur, fluorine, and chloride, such as ²H, ³H, ¹³C, ¹⁴C, ¹⁵N, ¹⁸O, ¹⁷O, ³¹P, ³²p ³⁵S, ¹⁸F, and ³⁶Cl, respectively. Compounds described herein, and the pharmaceutically acceptable salts, solvates, or stereoisomers thereof which contain the aforementioned isotopes and/or other isotopes of other atoms are within the scope of this disclosure. Certain isotopically-labeled compounds, for example those into which radioactive isotopes such as ³H and ¹⁴C are incorporated, are useful in drug and/or substrate tissue distribution assays. Tritiated, i.e., ³H and carbon-14, i.e., ¹⁴C, isotopes are particularly preferred for their ease of preparation and detectability. Further, substitution with heavy isotopes such as deuterium, i.e., 2H, produces certain therapeutic advantages resulting from greater metabolic stability, for example increased in vivo half-life or reduced dosage requirements.

In some embodiments, the compounds described herein are labeled by other means, including, but not limited to, the use of chromophores or fluorescent moieties, bioluminescent labels, or chemiluminescent labels.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the compounds disclosed herein, or a solvate, or stereoisomer thereof, or by separately reacting a purified compound in its free form with a suitable acid or base, and isolating the salt thus formed.

Examples of pharmaceutically acceptable salts include those salts prepared by reaction of the compounds described herein with a mineral, organic acid or inorganic base, such salts including, acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylateundeconate and xylenesulfonate.

Further, the compounds described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid and muconic acid. In some embodiments, other acids, such as oxalic, while not in themselves pharmaceutically acceptable, are employed in the preparation of salts useful as intermediates in obtaining the compounds disclosed herein, solvate, or stereoisomer thereof and their pharmaceutically acceptable acid addition salts.

In some embodiments, those compounds described herein which comprise a free acid group react with a suitable base, such as the hydroxide, carbonate, bicarbonate, sulfate, of a pharmaceutically acceptable metal cation, with ammonia, or with a pharmaceutically acceptable organic primary, secondary, tertiary, or quaternary amine. Representative salts include the alkali or alkaline earth salts, like lithium, sodium, potassium, calcium, and magnesium, and aluminum salts and the like. Illustrative examples of bases include sodium hydroxide, potassium hydroxide, choline hydroxide, sodium carbonate, N⁺(C₁₋₄ alkyl)₄, and the like.

Representative organic amines useful for the formation of base addition salts include ethylamine, diethylamine, ethylenediamine, ethanolamine, diethanolamine, piperazine and the like. It should be understood that the compounds described herein also include the quaternization of any basic nitrogen-containing groups they contain. In some embodiments, water or oil-soluble or dispersible products are obtained by such quaternization.

### Solvates

In some embodiments, the compounds described herein exist as solvates. The disclosure provides for methods of treating diseases by administering such solvates. The disclosure further provides for methods of treating diseases by administering such solvates as pharmaceutical compositions.

Solvates contain either stoichiometric or non-stoichiometric amounts of a solvent, and, in some embodiments, are formed with pharmaceutically acceptable solvents such as water, ethanol, and the like. Hydrates are formed when the solvent is water, or alcoholates are formed when the solvent is alcohol. Solvates of the compounds described herein can be conveniently prepared or formed during the processes described herein. By way of example only, hydrates of the compounds described herein can be conveniently prepared from an aqueous/organic solvent mixture, using organic solvents including, but not limited to, dioxane, tetrahydrofuran or methanol. In addition, the compounds provided herein can exist in unsolvated as well as solvated forms. In general, the solvated forms are considered equivalent to the unsolvated forms for the purposes of the compounds and methods provided herein.

### Tautomers

In some situations, compounds exist as tautomers. The compounds described herein include all possible tautomers within the formulas described herein. Tautomers are compounds that are interconvertible by migration of a hydrogen atom, accompanied by a switch of a single bond and adjacent double bond. In bonding arrangements where tautomerization is possible, a chemical equilibrium of the tautomers will exist. All tautomeric forms of the compounds disclosed herein are contemplated. The exact ratio of the tautomers depends on several factors, including temperature, solvent, and pH.

### Method of Treatment

Disclosed herein are methods of treatment of a disease in which inhibition of α₄β₇ integrin is beneficial, the method comprising administering a compound disclosed herein. Compounds inhibiting α₄β₇ are useful for development of medicaments to treat ulcerative colitis (UC) and Crohn's disease (CD). Inflammatory bowel diseases such as UC and CD are inflammatory diseases of the digestive tract and CD4⁺ memory T cells are implicated in disease pathogenesis via their ability to secrete pro-inflammatory, effector cytokines within the gut, impacting surrounding immune cells and tissue. The progression and flare ups of these diseases are believed to include extravasation of T cells leaving the blood to enter tissue in the gut resulting in inflammation via integrin related mechanisms. The inhibition of α₄β₇ can disrupt localization of T cells to gut tissue and the α₄β₇ targeting mAb, vedolizumab has been shown to be efficacious in the treatment of UC and CD. T cell homing to the gut requires surface expression of integrin α₄β₇ and chemokine receptor CCR9. While CCR9 is utilized by the cell to migrate towards the gradient of CCL25 expressed in the small intestine, α₄β₇ is a tethering molecule which binds the ligand, mucosal addressin cell adhesion molecule 1 (MAdCAM-1). Integrin α₄β₇ binds MAdCAM-1 with high affinity facilitating rolling and firm adhesion of cells followed by extravasation into tissue.

In some embodiments, the disease is inflammatory bowel disease, ileoanal anastomosis, eosinophilic esophagitis, pancreatitis, insulin-dependent diabetes mellitus, mastitis, cholecystitis, cholangitis, pericholangitis, chronic bronchitis, chronic sinusitis, asthma, graft versus host disease, or a chronic inflammatory disease of the lung.

In some embodiments, the disease is inflammatory bowel disease (IBD). In some embodiments, the inflammatory bowel disease is ulcerative colitis (UC) or Crohn's disease (CD).

In some embodiments, the inflammatory bowel disease is colitis, Crohn's disease, ileitis, Celiac disease, nontropical Sprue, enteropathy associated with seronegative arthropathies, gastroenteritis, or pouchitis.

In some embodiments, the disease is colitis. In some embodiments, the colitis is ulcerative colitis, microscopic colitis, or collagenous colitis.

In some embodiments, the disease is pouchitis; and the pouchitis is the result of proctocolectomy.

In some embodiments, the disease is gastroenteritis. In some embodiments, the gastroenteritis is eosinophilic gastroenteritis.

In some embodiments, the disease is eosinophilic esophagitis.

In some embodiments, the disease is a chronic inflammatory disease of the lung. In some embodiments, the chronic inflammatory disease of the lung is interstitial fibrosis.

In some embodiments, the interstitial fibrosis is hypersensitivity pneumonitis, collagen diseases, or sarcoidosis.

### Dosing

In certain embodiments, the compositions containing the compound(s) described herein are administered for prophylactic and/or therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation and/or dose ranging clinical trial.

In certain embodiments wherein the patient's condition does not improve, upon the doctor's discretion the administration of the compounds are administered chronically, that is, for an extended period of time, including throughout the duration of the patient's life in order to ameliorate or otherwise control or limit the symptoms of the patient's disease or condition.

Once improvement of the patient's conditions has occurred, a maintenance dose is administered if necessary. Subsequently, in specific embodiments, the dosage, or the frequency of administration, or both, is reduced, as a function of the symptoms, to a level at which the improved disease, disorder or condition is retained. In certain embodiments, however, the patient requires intermittent or daily treatment on a long-term basis upon any recurrence of symptoms.

The amount of a given agent that corresponds to such an amount varies depending upon factors such as the particular compound, disease condition and its severity, the identity (e.g., weight, sex) of the subject or host in need of treatment, but nevertheless is determined according to the particular circumstances surrounding the case, including, e.g., the specific agent being administered, the route of administration, the condition being treated, and the subject or host being treated.

Toxicity and therapeutic efficacy of such therapeutic regimens are determined by standard pharmaceutical procedures in cell cultures or experimental animals, including, but not limited to, the determination of the LD₁₀ and the ED₉₀. The dose ratio between the toxic and therapeutic effects is the therapeutic index and it is expressed as the ratio between LD₅₀ and ED₅₀. In certain embodiments, the data obtained from cell culture assays and animal studies are used in formulating the therapeutically effective daily dosage range and/or the therapeutically effective unit dosage amount for use in mammals, including humans. In some embodiments, the daily dosage amount of the compounds described herein lies within a range of circulating concentrations that include the ED₅₀ with minimal toxicity. In certain embodiments, the daily dosage range and/or the unit dosage amount varies within this range depending upon the dosage form employed and the route of administration utilized.

### Routes of Administration

Suitable routes of administration include, but are not limited to, oral, intravenous, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

In certain embodiments, a compound as described herein is administered in a local rather than systemic manner, for example, via injection of the compound directly into an organ, often in a depot preparation or sustained release formulation. In specific embodiments, long-acting formulations are administered by implantation (for example subcutaneously or intramuscularly) or by intramuscular injection. Furthermore, in other embodiments, the drug is delivered in a targeted drug delivery system, for example, in a liposome coated with organ specific antibody. In such embodiments, the liposomes are targeted to and taken up selectively by the organ. In yet other embodiments, the compound as described herein is provided in the form of a rapid release formulation, in the form of an extended-release formulation, or in the form of an intermediate release formulation. In yet other embodiments, the compound described herein is administered topically.

### Pharmaceutical Compositions/Formulations

The compounds described herein are administered to a subject in need thereof, either alone or in combination with pharmaceutically acceptable carriers, excipients, or diluents, in a pharmaceutical composition, according to standard pharmaceutical practice. In one embodiment, the compounds of this disclosure may be administered to animals. The compounds can be administered orally or parenterally, including the intravenous, intramuscular, intraperitoneal, subcutaneous, rectal, and topical routes of administration.

In another aspect, provided herein are pharmaceutical compositions comprising a compound described herein, or a pharmaceutically acceptable salt, solvate, or stereoisomer thereof, and at least one pharmaceutically acceptable excipient. Pharmaceutical compositions are formulated in a conventional manner using one or more pharmaceutically acceptable excipients that facilitate processing of the active compounds into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

In some embodiments, the pharmaceutically acceptable excipient is selected from carriers, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, and any combinations thereof.

The pharmaceutical compositions described herein are administered to a subject by appropriate administration routes, including, but not limited to, oral, parenteral (e.g., intravenous, subcutaneous, intramuscular), intranasal, buccal, topical, rectal, or transdermal administration routes. The pharmaceutical formulations described herein include, but are not limited to, aqueous liquid dispersions, liquids, gels, syrups, elixirs, slurries, suspensions, self-emulsifying dispersions, solid solutions, liposomal dispersions, aerosols, solid oral dosage forms, powders, immediate release formulations, controlled release formulations, fast melt formulations, tablets, capsules, pills, powders, dragees, effervescent formulations, lyophilized formulations, delayed release formulations, extended release formulations, pulsatile release formulations, multiparticulate formulations, and mixed immediate and controlled release formulations.

Pharmaceutical compositions including compounds described herein, or a pharmaceutically acceptable salt, solvate, or stereoisomer thereof are manufactured in a conventional manner, such as, by way of example only, by means of conventional mixing, dissolving, granulating, dragee-making, levigating, emulsifying, encapsulating, entrapping, or compression processes.

Pharmaceutical compositions for oral use are obtained by mixing one or more solid excipient with one or more of the compounds described herein, optionally grinding the resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as polyvinylpyrrolidone (PVP or povidone) or calcium phosphate. If desired, disintegrating agents are added, such as the cross-linked croscarmellose sodium, polyvinylpyrrolidone, agar, or alginic acid or a salt thereof such as sodium alginate. In some embodiments, dyestuffs or pigments are added to the tablets or dragee coatings for identification or to characterize different combinations of active compound doses.

Pharmaceutical compositions that are administered orally include push-fit capsules made of gelatin, as well as soft, sealed capsules made of gelatin and a plasticizer, such as glycerol or sorbitol. The push-fit capsules contain the active ingredients in admixture with filler such as lactose, binders such as starches, and/or lubricants such as talc or magnesium stearate and, optionally, stabilizers. In soft capsules, the active compounds are dissolved or suspended in suitable liquids, such as fatty oils, liquid paraffin, or liquid polyethylene glycols. In some embodiments, stabilizers are added.

Pharmaceutical compositions for parental use are formulated as infusions or injections. In some embodiments, the pharmaceutical composition suitable for injection or infusion includes sterile aqueous solutions, or dispersions, or sterile powders comprising a compound described herein, or a pharmaceutically acceptable salt, solvate, or stereoisomer thereof. In some embodiments, the pharmaceutical composition comprises a liquid carrier. In some embodiments, the liquid carrier is a solvent or liquid dispersion medium comprising, for example, water, saline, ethanol, a polyol (for example, glycerol, propylene glycol, liquid polyethylene glycols, and the like), vegetable oils, nontoxic glyceryl esters, and any combinations thereof. In some embodiments, the pharmaceutical compositions further comprise a preservative to prevent growth of microorganisms.

### Combination

Disclosed herein are methods of treating cancer using a compound disclosed herein, or a pharmaceutically acceptable salt, solvate, or stereoisomer thereof, in combination with an additional therapeutic agent.

In some embodiments, the additional therapeutic agent is administered at the same time as the compound disclosed herein. In some embodiments, the additional therapeutic agent and the compound disclosed herein are administered sequentially. In some embodiments, the additional therapeutic agent is administered less frequently than the compound disclosed herein. In some embodiments, the additional therapeutic agent is administered more frequently than the compound disclosed herein. In some embodiments, the additional therapeutic agent is administered prior than the administration of the compound disclosed herein. In some embodiments, the additional therapeutic agent is administered after the administration of the compound disclosed herein.

### EXAMPLES

### Synthesis of the Compounds

The compounds may be prepared using the methods disclosed herein and routine modifications thereof, which will be apparent given the disclosure herein and methods well known in the art. Conventional and well-known synthetic methods may be used in addition to the teachings herein. The synthesis of typical compounds described herein may be accomplished as described in the following examples. If available, reagents and starting materials may be purchased commercially, e.g., from Sigma Aldrich or other chemical suppliers.

It will be appreciated that where typical process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, conventional protecting groups (“PG”) may be necessary to prevent certain functional groups from undergoing undesired reactions. Suitable protecting groups for various functional groups as well as suitable conditions for protecting and deprotecting particular functional groups are well known in the art. For example, numerous protecting groups are described in Wuts, P. G. M., Greene, T. W., & Greene, T. W. (2006). Greene's protective groups in organic synthesis. Hoboken, N.J., Wiley-Interscience, and references cited therein. For example, protecting groups for alcohols, such as hydroxy, include silyl ethers (including trimethylsilyl (TMS), tert-butyldimethylsilyl (TBDMS), tri-iso-propylsilyloxymethyl (TOM), and triisopropylsilyl (TIPS) ethers), which can be removed by acid or fluoride ion, such as NaF, TBAF (tetra-n-butylammonium fluoride), HF-Py, or HF-NEt₃. Other protecting groups for alcohols include acetyl, removed by acid or base, benzoyl, removed by acid or base, benzyl, removed by hydrogenation, methoxyethoxymethyl ether, removed by acid, dimethoxytrityl, removed by acid, methoxymethyl ether, removed by acid, tetrahydropyranyl or tetrahydrofuranyl, removed by acid, and trityl, removed by acid. Examples of protecting groups for amines include carbobenzyloxy, removed by hydrogenolysis p-methoxybenzyl carbonyl, removed by hydrogenolysis, tert-butyloxycarbonyl, removed by concentrated strong acid (such as HCl or CF₃COOH), or by heating to greater than about 80° C., 9-fluorenylmethyloxycarbonyl, removed by base, such as piperidine, acetyl, removed by treatment with a base, benzoyl, removed by treatment with a base, benzyl, removed by hydrogenolysis, carbamate group, removed by acid and mild heating, p-methoxybenzyl, removed by hydrogenolysis, 3,4-dimethoxybenzyl, removed by hydrogenolysis, p-methoxyphenyl, removed by ammonium cerium(IV) nitrate, tosyl, removed by concentrated acid (such as HBr or H₂SO₄) and strong reducing agents (sodium in liquid ammonia or sodium naphthalenide), troc (trichloroethyl chloroformate), removed by Zn insertion in the presence of acetic acid, and sulfonamides (Nosyl & Nps), removed by samarium iodide or tributyltin hydride.

Furthermore, the compounds of this disclosure may contain one or more chiral centers. Accordingly, if desired, such compounds can be prepared or isolated as pure stereoisomers, i.e., as individual enantiomers or diastereomers or as stereoisomer-enriched mixtures. All such stereoisomers (and enriched mixtures) are included within the scope of this disclosure, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents, and the like.

The starting materials for the following reactions are generally known compounds or can be prepared by known procedures or obvious modifications thereof. For example, many of the starting materials are available from commercial suppliers such as Aldrich Chemical Co. (Milwaukee, Wisconsin, USA), Bachem (Torrance, California, USA), Emka-Chemce or Sigma (St. Louis, Missouri, USA). Others may be prepared by procedures or obvious modifications thereof, described in standard reference texts such as Fieser and Fieser's Reagents for Organic Synthesis, Volumes 1-15 (John Wiley, and Sons, 1991), Rodd's Chemistry of Carbon Compounds, Volumes 1-5, and Supplementals (Elsevier Science Publishers, 1989) organic Reactions, Volumes 1-40 (John Wiley, and Sons, 1991), March's Advanced Organic Chemistry, (John Wiley, and Sons, 5th Edition, 2001), and Larock's Comprehensive Organic Transformations (VCH Publishers Inc., 1989).

### General Synthesis

Scheme I illustrates general methods which can be employed for the synthesis of compounds described herein, where n, m, p, X, ring A, ring B, ring C, R¹, R², R³, R⁴, R⁵, R⁶, R⁸, and R⁹ are each independently as defined herein; LG is independently a leaving group (e.g., halo, —OTf, etc.); and PG is a suitable protecting group (e.g., alkyl such as methyl, ethyl, tert-butyl, etc.).

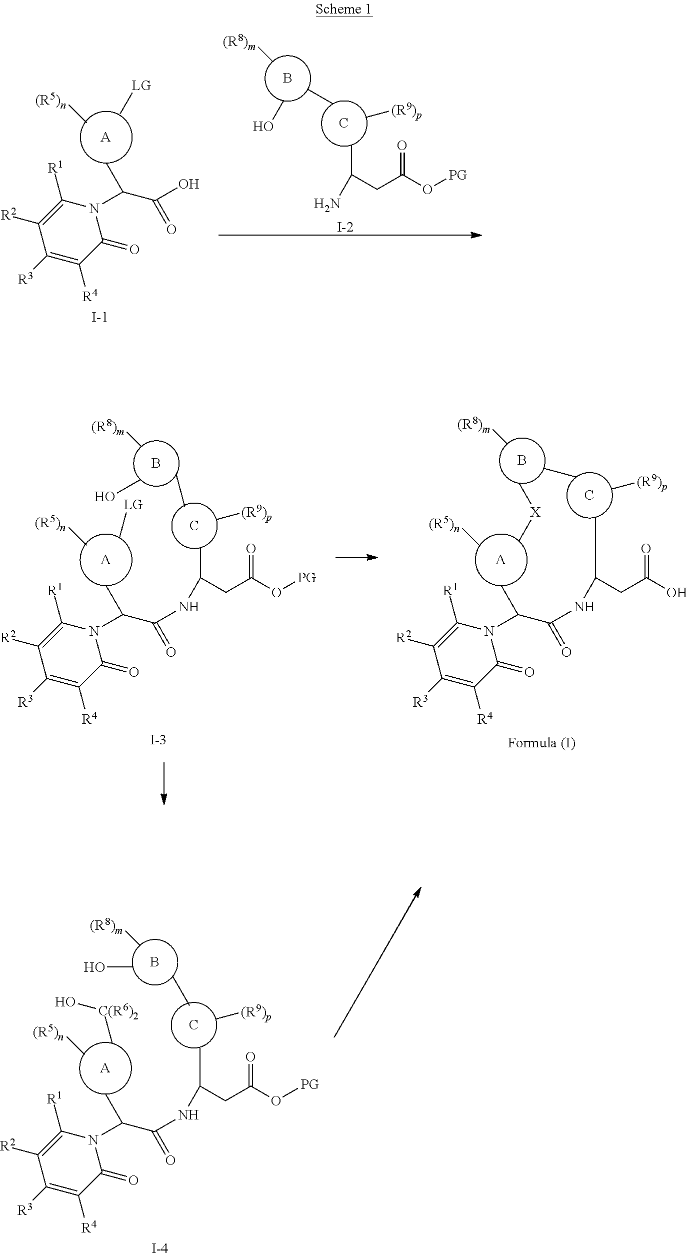

In Scheme 1, compounds of Formula (I) may be prepared by contacting compound I-1 with compound I-2 under suitable conditions, such as in the presence of a coupling reagent (such as 1-ethyl-3-(3-dimethylaminopropyl)carbodiimide (EDCI), 1-hydroxybenzotriazole (HOBT), 1-[bis(dimethylamino)methylene]-1H-1,2,3-triazolo[4,5-b]pyridinium 3-oxide hexafluorophosphate (HATU), and the like), and optionally a base, to provide compound I-3.

Compound I-3 may then undergo cyclization in the presence of a catalyst (such as copper iodide, sodium iodide, and the like) and a base (such as potassium carbonate, sodium carbonate, cesium carbonate, and the like), followed by deprotection of the carboxylic acid to provide compounds of Formula (I) wherein X is —O—.

Alternatively, compound I-3 may be further undergo Stille coupling in the presence of an organotin reagent (such as (tributyltin)methanol) and a palladium catalyst to provide compounds I-4. Compound I-4 may then undergo dehydration at elevated temperatures of about 50-120° C. in a suitable solvent (such as toluene) to cyclize and provide compounds of Formula (I) wherein X is —C(R⁶)₂O—.

Upon each reaction completion, each of the intermediate or final compounds can be recovered, and optionally purified, by conventional techniques such as neutralization, extraction, precipitation, chromatography, filtration and the like.

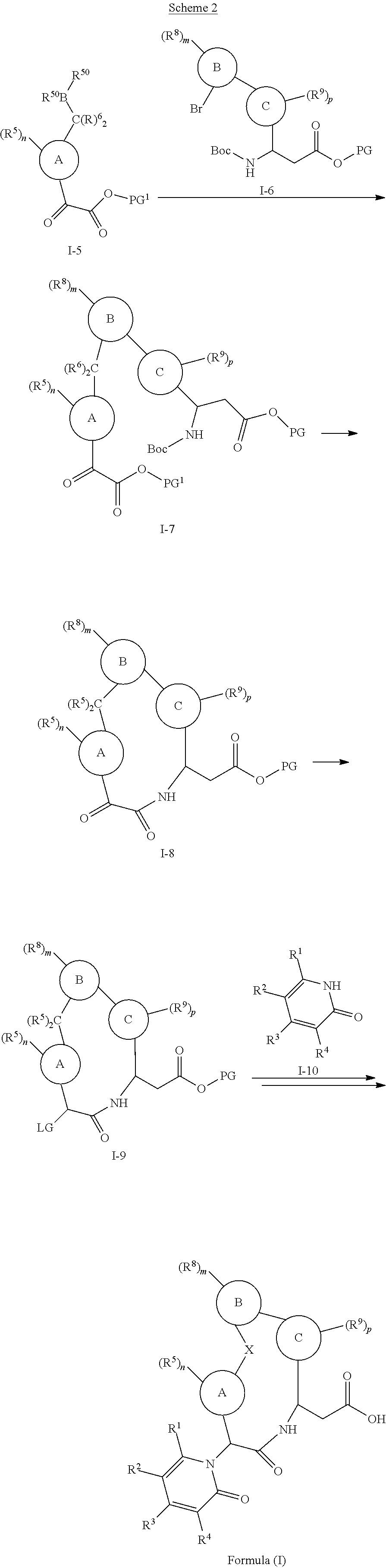

Alternatively, compounds of Formula (I) may be prepared by the route shown in Scheme 2, where n, m, p, X, ring A, ring B, ring C, R¹, R², R³, R⁴, R⁵, R⁶, R⁸, and R⁹ are each independently as defined herein; PG and PG¹ are suitable protecting groups (e.g., alkyl such as methyl, ethyl, tert-butyl, etc.), provided that PG and PG¹ are different; LG is a suitable leaving group (e.g., mesylate, tosylate, triflate, etc.); and each R⁵⁰ is independently —OH, —O-alkyl, or together with the boron atom to which they are attached, form a cyclic boronate.

In Scheme 2, compounds of Formula (I) may be prepared by first contacting compound I-5 with compound I-6 under suitable Suzuki coupling conditions (i.e., in the presence of a palladium catalyst such as XPhos Pd G3, and a base such as potassium carbonate, sodium carbonate, cesium carbonate, and the like) to provide compound I-7.

Compound I-7 may then undergo deprotection under acidic or basic conditions to provide the amine, followed by amide coupling in the presence of a suitable coupling reagent (such as chloro-N,N,N′,N′-tetramethylformamidinium hexafluorophosphate (TCFH) and 1-methylimidazole (NMI), and the like) to provide compound I-8.

The ketone in compound I-8 may then be converted to an alcohol in the presence of a suitable reducing agent (such as sodium borohydride, lithium aluminum hydride, and the like). The alcohol may then be converted to a suitable leaving group contacting with a suitable reagent, such as mesyl chloride, tosyl chloride, and the like, to provide compound I-9.

Finally, compound I-9 is contacted with compound I-10 in the presence of a base (such as potassium carbonate, sodium carbonate, or cesium carbonate), followed by deprotection under acidic or basic conditions, to provide compounds of Formula (I) wherein X is —C(R⁶)₂—.

Upon each reaction completion, each of the intermediate or final compounds can be recovered, and optionally purified, by conventional techniques such as neutralization, extraction, precipitation, chromatography, filtration and the like.

The reagents and starting materials used in Scheme I and Scheme 2 can be purchased from commercial sources or prepared according to methods known to the skilled artisan.

### Synthesis of Intermediates

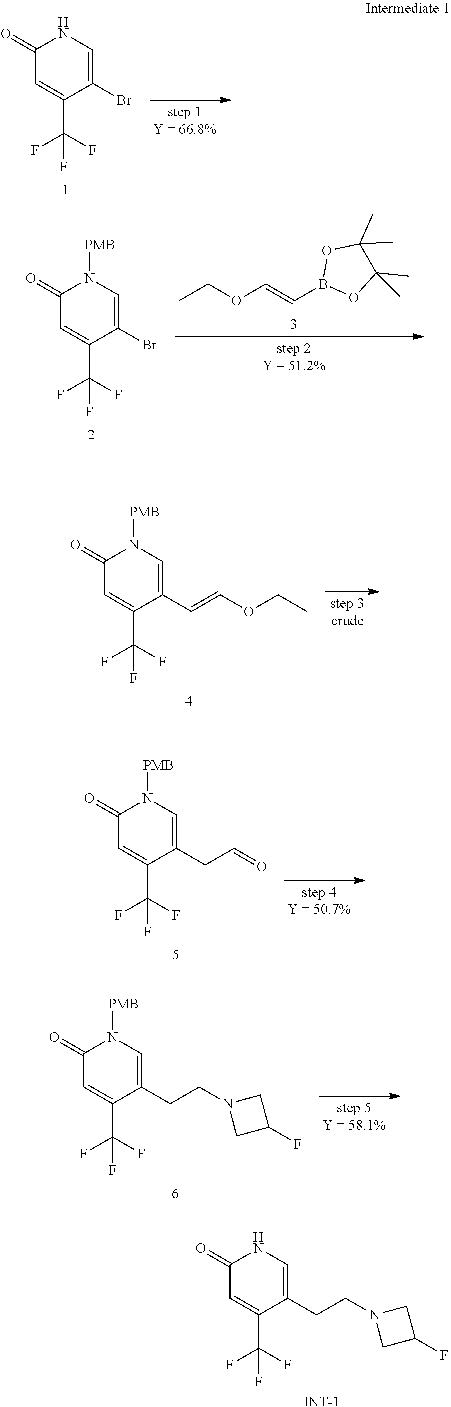

### Step 1: Preparation of 5-bromo-1-[(4-methoxyphenyl)methyl]-4-(trifluoromethyl)pyridin-2-one

To a stirred mixture of 5-bromo-4-(trifluoromethyl)-1H-pyridin-2-one (50.00 g, 206.61 mmol, 1.00 equiv) and PMBCl (48.00 g, 309.92 mmol, 1.50 equiv) in DMF (600 mL) was added Cs₂CO₃ (134.00 g, 413.23 mmol, 2.00 equiv) at room temperature under nitrogen atmosphere. The resulting mixture was stirred overnight at 60° C. under nitrogen atmosphere and then cooled to room temperature. The resulting mixture was filtered, and the filter cake was washed with EtOAc (800 mL). The resulting mixture was extracted with water (3×900 mL). The combined organic layers were washed with brine (2×800 mL), dried over anhydrous Na₂SO₄, filtered, and concentrated. The residue was purified by silica gel column chromatography, eluted with PE/EA (20%40%, 25 min), to afford 5-bromo-1-[(4-methoxyphenyl)methyl]-4-(trifluoromethyl)pyridin-2-one (50 g, 66.8%) as a yellow solid. LC-MS: (ES+H, m/z): [M+H]⁺=362.1; ¹H NMR (300 MHz, DMSO-d₆) δ 8.48 (s, 1H), 7.42-7.27 (m, 2H), 6.97-6.84 (m, 3H), 5.04 (s, 2H), 3.73 (s, 3H). ¹⁹F NMR (300 MHz, DMSO-d₆) δ −64.13.

### Step 2: Preparation of 5-[(E)-2-ethoxyethenyl]-1-[(4-methoxyphenyl)methyl]-4-(trifluoromethyl)pyridin-2-one

To a stirred mixture of 5-bromo-1-[(4-methoxyphenyl)methyl]-4-(trifluoromethyl)pyridin-2-one (50.00 g, 138.06 mmol, 1.00 equiv) and 2-[(E)-2-ethoxyethenyl]-4,4,5,5-tetramethyl-1,3,2-dioxaborolane (30.00 g, 151.87 mmol, 1.10 equiv) in 1,4-dioxane (600 mL) and H₂O (30 mL) were added Pd(dppf)Cl₂ (5.00 g, 6.90 mmol, 0.05 equiv) and Cs₂CO₃ (134.00 g, 414.19 mmol, 3.00 equiv) at room temperature under nitrogen atmosphere. The resulting mixture was stirred overnight at 70° C. under nitrogen atmosphere and then cooled to room temperature. The resulting mixture was filtered and the filter cake was washed with EtOAc (800 mL). The resulting mixture was extracted with water (3×900 mL). The combined organic layers were washed with brine (2×800 mL), dried over anhydrous Na₂SO₄, filtered, and concentrated. The residue was purified by silica gel column chromatography, eluted with PE/EA (20%˜40%, 30 min), to afford 5-[(E)-2-ethoxyethenyl]-1-[(4-methoxyphenyl)methyl]-4-(trifluoromethyl)pyridin-2-one (25 g, 51.2%) as a yellow oil. LC-MS: (ES+H, m/z): [M+H]⁺=354.1; ¹H NMR (400 MHz, DMSO-d₆) δ 8.06 (d, J=6.7 Hz, 1H), 7.39-7.27 (m, 2H), 6.96-6.74 (m, 4H), 5.56-5.49 (m, 1H), 5.07 (d, J=10.0 Hz, 2H), 3.86 (q, J=7.0 Hz, 2H), 3.73 (s, 3H), 1.27-1.16 (m, 3H). ¹⁹F NMR (300 MHz, DMSO-d₆) δ −62.96.

### Step 3: Preparation of 2-{1-[(4-methoxyphenyl)methyl]-6-oxo-4-(trifluoromethyl)pyridin-3-yl}acetaldehyde

To a stirred mixture of 5-[(E)-2-ethoxyethenyl]-1-[(4-methoxyphenyl)methyl]-4-(trifluoromethyl)pyridin-2-one (25.00 g, 70.75 mmol, 1.00 equiv) in CH₂Cl₂ (80 mL) was added TFA (80 mL) at room temperature under nitrogen atmosphere. The resulting mixture was stirred for 5 h at room temperature under nitrogen atmosphere. The reaction was monitored by LCMS. Desired product could be detected by LCMS. The resulting mixture was concentrated under reduced pressure. The resulting mixture was diluted with CH₂Cl₂ (300 mL). The resulting mixture was washed with NaHCO₃ (aq. 3×300 mL). The combined organic layers were washed with brine (2×300 mL), dried over anhydrous Na₂SO₄, filtered and concentrated to afford 2-{1-[(4-methoxyphenyl)methyl]-6-oxo-4-(trifluoromethyl)pyridin-3-yl}acetaldehyde as a red oil. The crude product was used in the next step directly without further purification. LC-MS: (ES+H, m/z): [M+H]⁺=326.1.

### Step 4: Preparation of 5-[2-(3-fluoroazetidin-1-yl)ethyl]-1-[(4-methoxyphenyl)methyl]-4-(trifluoromethyl)pyridin-2-one

To a stirred mixture of 2-{1-[(4-methoxyphenyl)methyl]-6-oxo-4-(trifluoromethyl)pyridin-3-yl}acetaldehyde (25.00 g, 76.85 mmol, 1.00 equiv, crude) and 3-fluoroazetidine (7.50 g, 99.91 mmol, 1.30 equiv) in EtOH (400 mL) were added CH₃COOH (461 mg, 7.68 mmol, 0.10 equiv) at room temperature under nitrogen atmosphere. The resulting mixture was stirred for 1 h at room temperature under nitrogen atmosphere. To the above mixture was added NaBH₃CN (9.66 g, 153.71 mmol, 2.00 equiv) in portions over 10 min at room temperature. The resulting mixture was stirred for an additional 4 h at room temperature. The reaction was monitored by LCMS. Desired product could be detected by LCMS. The reaction was quenched by the addition of water/ice (500 mL) at 0° C. The resulting mixture was extracted with EtOAc (3×600 mL) and the combined organic layers were washed with brine (2×500 mL), dried over anhydrous Na₂SO₄, filtered and concentrated. The crude product was purified by silica gel column chromatography, eluted with CH₂Cl₂/MeOH (0˜10%, 25 min), to afford 5-[2-(3-fluoroazetidin-1-yl)ethyl]-1-[(4-methoxyphenyl)methyl]-4-(trifluoromethyl)pyridin-2-one (15 g, 50.7%) as a yellow oil. LC-MS: (ES+H, m/z): [M+H]⁺=385.1; ¹H NMR (300 MHz, DMSO-d₆) δ 7.96 (s, 1H), 7.36-7.29 (m, 2H), 6.95-6.87 (m, 2H), 6.74 (s, 1H), 5.25-5.15 (m, 1H), 5.04 (s, 2H), 3.73 (s, 3H), 3.60-3.48 (m, 2H), 3.15-2.99 (m, 2H), 2.57 (t, J=7.3 Hz, 2H), 2.42 (t, J=7.3 Hz, 2H). ¹⁹F NMR (300 MHz, DMSO-d₆) δ −62.51, −177.55.

### Step 5: Preparation of 5-[2-(3-fluoroazetidin-1-yl)ethyl]-4-(trifluoromethyl)-1H-pyridin-2-one

A stirred mixture of 5-[2-(3-fluoroazetidin-1-yl)ethyl]-1-[(4-methoxyphenyl)methyl]-4-(trifluoromethyl)pyridin-2-one (15.00 g, 39.02 mmol, 1.00 equiv) in TFA (80 mL) was stirred overnight at 80° C. under nitrogen atmosphere. The reaction was monitored by LCMS. Upon completion, the mixture was allowed to cool down to room temperature. The resulting mixture was concentrated under vacuum. The residue was purified by reversed-phase flash chromatography (C18; MeOH in Water (10 mM NH₄HCO₃), 10%-50%, UV 254 nm) to afford 5-[2-(3-fluoroazetidin-1-yl)ethyl]-4-(trifluoromethyl)-1H-pyridin-2-one (6 g, 58.1%) as an off-white solid. LC-MS: (ES+H, m/z): [M+H]⁺=265.1; ¹H NMR (400 MHz, DMSO-d₆) δ 12.12 (s, 1H), 7.53 (s, 1H), 6.67 (s, 1H), 5.25-5.03 (m, 1H), 3.63-3.50 (m, 2H), 3.15-3.01 (m, 2H), 2.57 (t, J=7.4 Hz, 2H), 2.42 (t, J=7.4 Hz, 2H). ¹⁹F NMR (400 MHz, DMSO-d₆) δ −62.61, −177.61.

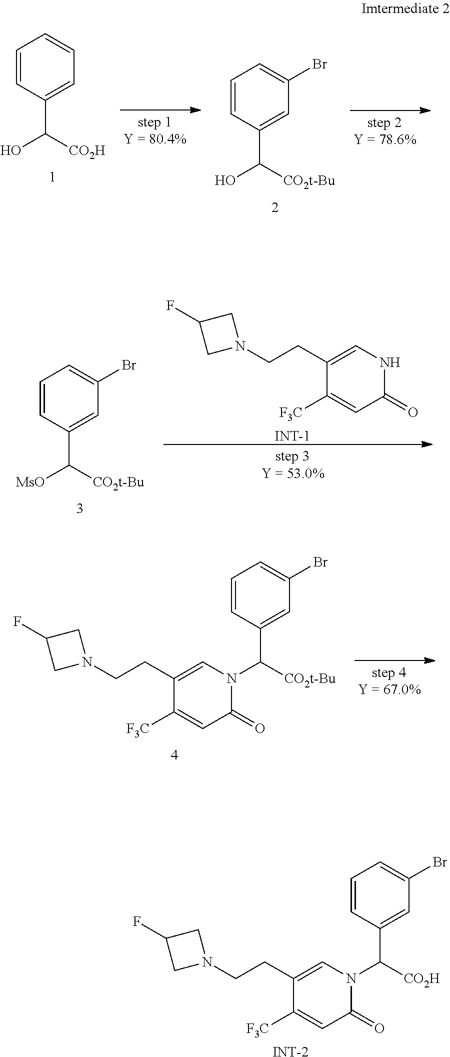

### Step 1: Preparation of tert-butyl 2-(3-bromophenyl)-2-hydroxyacetate

To a stirred solution of (3-bromophenyl)(hydroxy)acetic acid (5.00 g, 21.64 mmol, 1.00 equiv) in tetrahydrofuran (100 mL) was added N,N′-diisopropyltert-butoxymethanimidamide (13.01 g, 64.92 mmol, 3.00 equiv) dropwise at 0° C. under nitrogen atmosphere. The resulting mixture was stirred for 3 h at 0° C. under nitrogen atmosphere. The reaction was monitored by TLC (PE/EA, 5/1). Upon completion, the reaction mixture was concentrated under reduced pressure. The residue was purified by silica gel column chromatography, eluted with PE/EA (0% to 20% gradient in 30 min), to afford tert-butyl 2-(3-bromophenyl)-2-hydroxyacetate (5 g, 80.4%) as a colorless oil. ¹H NMR (300 MHz, CDCl₃) δ 7.59 (t, J=1.9 Hz, 1H), 7.46-7.42 (m, 1H), 7.38-7.33 (m, 1H), 7.22 (t, J=7.8 Hz, 1H), 5.01 (d, J=3.3 Hz, 1H), 3.63-3.47 (m, 1H), 1.42 (s, 9H).

### Step 2: Preparation of tert-butyl 2-(3-bromophenyl)-2-(methanesulfonyloxy)acetate

To a stirred solution of tert-butyl 2-(3-bromophenyl)-2-hydroxyacetate (4.00 g, 13.93 mmol, 1.00 equiv) and Et₃N (2.26 g, 22.33 mmol, 1.60 equiv) in DCM (6 mL) was added MsCl (2.07 g, 18.07 mmol, 1.30 equiv) dropwise at 0° C. under nitrogen atmosphere. The resulting mixture was stirred for 5 h at 0° C. to RT under nitrogen atmosphere. The reaction was monitored by TLC (PE/EA, 10/1). The resulting mixture was concentrated under reduced pressure and the crude product was purified by silica gel column chromatography (PE/EA (0% to 20% gradient in 30 min)) to afford tert-butyl 2-(3-bromophenyl)-2-(methanesulfonyloxy)acetate (4 g, 78.6%) as a white solid. ¹H NMR (300 MHz, CDCl₃) δ 7.60 (d, J=1.9 Hz, 1H), 7.57-7.51 (m, 1H), 7.40-7.37 (m, 1H), 7.29 (d, J=7.8 Hz, 1H), 5.76 (s, 1H), 3.15 (s, 3H), 1.44 (s, 9H).

### Step 3: Preparation of tert-butyl 2-(3-bromophenyl)-2-{5-[2-(3-fluoroazetidin-1-yl)ethyl]-2-oxo-4-(trifluoromethyl)pyridin-1-yl}acetate

To a stirred mixture of tert-butyl 2-(3-bromophenyl)-2-(methanesulfonyloxy)acetate (5.00 g, 13.69 mmol, 1.29 equiv) and 5-[2-(3-fluoroazetidin-1-yl)ethyl]-4-(trifluoromethyl)-1H-pyridin-2-one (2.80 g, 10.59 mmol, 1.00 equiv) in CH₃CN (200 mL) was added K₂CO₃ (4.39 g, 31.79 mmol, 3.00 equiv) in portions at room temperature under nitrogen atmosphere. The resulting mixture was stirred for 6 h at 80° C. under nitrogen atmosphere. The reaction was monitored by LCMS. The mixture was allowed to cool down to room temperature and concentrated. The resulting mixture was diluted with EtOAc (500 mL), washed with water (2×50 mL), dried over anhydrous Na₂SO₄, filtered and concentrated. The crude product was purified by silica gel column chromatography (PE/EA (0% to 30%)) to afford tert-butyl 2-(3-bromophenyl)-2-{5-[2-(3-fluoroazetidin-1-yl)ethyl]-2-oxo-4-(trifluoromethyl)pyridin-1-yl}acetate (3 g, 53.0%) as a white solid. LC-MS: (ES+H, m/z): [M+H]⁺=533.1; ¹H NMR (300 MHz, DMSO-d₆) δ 7.74-7.66 (m, 2H), 7.52-7.42 (m, 3H), 6.85 (s, 1H), 6.27 (s, 1H), 5.15-4.89 (m, 1H), 3.46-3.36 (m, 2H), 3.03-2.88 (m, 2H), 2.51-2.46 (m, 2H), 2.39 (t, J=6.6 Hz, 2H), 1.41 (s, 9H).

### Step 4: Preparation of (3-bromophenyl)({5-[2-(3-fluoroazetidin-1-yl)ethyl]-2-oxo-4-(trifluoromethyl)pyridin-1-yl})acetic acid

To a stirred solution of tert-butyl 2-(3-bromophenyl)-2-{5-[2-(3-fluoroazetidin-1-yl)ethyl]-2-oxo-4-(trifluoromethyl)pyridin-1-yl}acetate (3.00 g, 5.62 mmol, 1.00 equiv) in DCM (15 mL) was added TFA (15 mL) dropwise at 0° C. under nitrogen atmosphere. The resulting mixture was stirred for 3 h at room temperature under nitrogen atmosphere. The reaction was monitored by LCMS. The resulting mixture was concentrated under reduced pressure and the crude product was purified by reversed-phase flash chromatography (C18 gel; MeCN in water (0.1% NH₃H₂O), 0% to 70% gradient in 40 min; UV 254 nm) to afford (3-bromophenyl)({5-[2-(3-fluoroazetidin-1-yl)ethyl]-2-oxo-4-(trifluoromethyl)pyridin-1-yl})acetic acid (1.8 g, 67.0%) as a white solid. LC-MS: (ES+H, m/z): [M+H]⁺=477.1; ¹H NMR (400 MHz, DMSO-d₆) δ 7.76-7.66 (m, 2H), 7.63-7.60 (m, 1H), 7.46-7.35 (m, 2H), 6.83 (s, 1H), 6.35 (s, 1H), 5.19-5.00 (m, 1H), 3.70-3.61 (m, 4H), 2.73-2.65 (m, 2H), 2.47 (d, J=7.2 Hz, 2H).

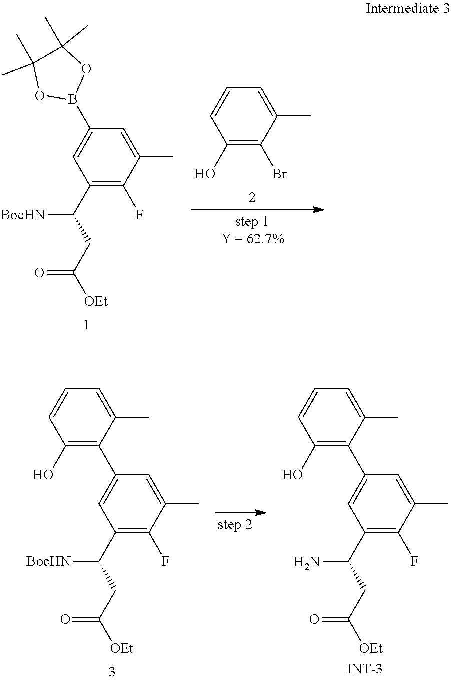

### Step 1: Preparation of ethyl (3S)-3-[(tert-butoxycarbonyl)amino]-3-{4-fluoro-2′-hydroxy-5,6′-dimethyl-[1,1′-biphenyl]-3-yl}propanoate

To a stirred solution of ethyl (3S)-3-[(tert-butoxycarbonyl)amino]-3-[2-fluoro-3-methyl-5-(4,4,5,5-tetramethyl-1,3,2-dioxaborolan-2-yl)phenyl]propanoate (prepared according to WO2021076890) (1.00 g, 2.21 mmol, 1.00 equiv) and 2-bromo-3-methylphenol (0.50 g, 2.65 mmol, 1.20 equiv) in dioxane (10 mL) was added Pd(PPh₃)₄ (256 mg, 0.22 mmol, 0.10 equiv) and K₂HPO₄ (1.16 g, 6.64 mmol, 3.00 equiv) dropwise at room temperature under nitrogen atmosphere. The resulting mixture was stirred for overnight at 90° C. under nitrogen atmosphere and then cooled to room temperature. The resulting mixture was diluted with EtOAc (200 mL), washed with water (2×20 mL), dried over anhydrous Na₂SO₄, filtered and concentrated. The crude product was purified by reversed-phase flash chromatography (C18; mobile phase, MeCN in water (0.1% NH₃H₂O), 0% to 60% gradient in 40 min; detector, UV 254 nm) to afford ethyl (3S)-3-[(tert-butoxycarbonyl)amino]-3-{4-fluoro-2′-hydroxy-5,6′-dimethyl-[1,1′-biphenyl]-3-yl}propanoate (600 mg, 62.7%) as a colorless oil. LC-MS: (ES+H, m/z): [M+H]⁺=432.2; ¹H NMR (300 MHz, DMSO-d₆) δ 9.09 (s, 1H), 7.51 (d, J=9.1 Hz, 2H), 7.03 (t, J=7.6 Hz, 2H), 6.73 (t, J=8.6 Hz, 2H), 5.28 (d, J=8.4 Hz, 1H), 4.06-4.01 (m, 2H), 2.66 (d, J=7.3 Hz, 2H), 2.24 (d, J=2.1 Hz, 3H), 1.95 (s, 3H), 1.35 (d, J=4.5 Hz, 9H), 1.18-1.13 (m, 3H).

### Step 2: Preparation of ethyl (3S)-3-amino-3-{4-fluoro-2′-hydroxy-5,6′-dimethyl-[1,1′-biphenyl]-3-yl}propanoate

To a stirred solution of ethyl (3S)-3-[(tert-butoxycarbonyl)amino]-3-{4-fluoro-2′-hydroxy-5,6′-dimethyl-[1,1′-biphenyl]-3-yl}propanoate (600 mg, 1.39 mmol, 1 equiv) in DCM (6 mL) was added HCl (gas) in 1,4-dioxane (3 mL, 4 M) dropwise at 0° C. under nitrogen atmosphere. The resulting mixture was stirred for 3 h at room temperature under nitrogen atmosphere. The reaction was monitored by LCMS. Upon completion, the resulting mixture was concentrated under reduced pressure to provide ethyl (3S)-3-amino-3-{4-fluoro-2′-hydroxy-5,6′-dimethyl-[1,1′-biphenyl]-3-yl}propanoate (600 mg, crude). LC-MS: (ES+H, m/z): [M+H]⁺=332.1.

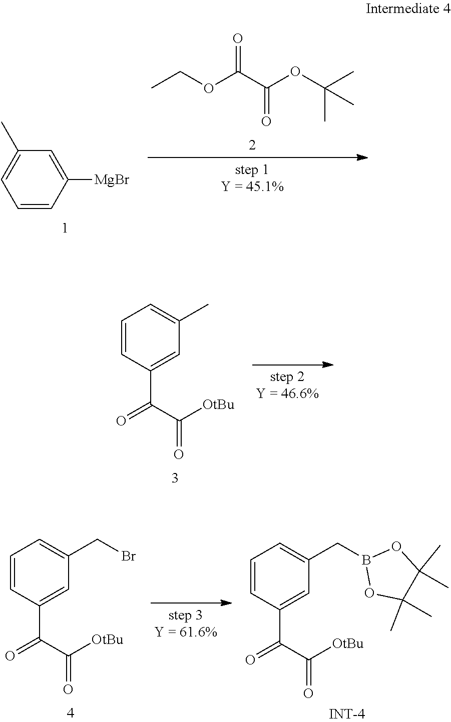

### Step 1: Preparation of tert-butyl 2-(3-methylphenyl)-2-oxoacetate

To a stirred solution of tert-butyl ethyl oxalate (10.00 g, 57.40 mmol, 1.00 equiv.) in THF (100 mL) was added bromo(3-methylphenyl)magnesium (68 mL, 68.88 mmol, 1.20 equiv, 1M/L in THF) dropwise at −78° C. under nitrogen atmosphere. The resulting mixture was stirred for 3 h at −78° C. under nitrogen atmosphere. The reaction was monitored by TLC (PE/EA=20/1, r=0.5). The reaction was quenched by the addition of sat. NH₄Cl (aq.) (10 mL) at −78° C. The resulting mixture was concentrated under reduced pressure and the crude product was purified by silica gel column chromatography (PE/EA (0% to 5% gradient in 20 min)) to afford tert-butyl 2-(3-methylphenyl)-2-oxoacetate (6.00 g, 45.1%) as a yellow oil. ¹H NMR (300 MHz, DMSO-d₆) δ 7.72-7.65 (m, 2H), 7.63-7.49 (m, 2H), 2.41 (s, 3H), 1.59 (s, 9H).

### Step 2: Preparation of tert-butyl 2-[3-(bromomethyl)phenyl]-2-oxoacetate

To a stirred solution of tert-butyl 2-(3-methylphenyl)-2-oxoacetate (6.00 g, 27.24 mmol, 1.00 equiv.) and NBS (5.82 g, 32.68 mmol, 1.20 equiv.) in MeCN (50 mL) was added AIBN (0.45 g, 2.72 mmol, 0.10 equiv.) at room temperature under nitrogen atmosphere. The resulting mixture was stirred overnight at 60° C. under nitrogen atmosphere. The reaction was monitored by TLC (PE/EA=20/1, r=0.7). The resulting mixture was concentrated under reduced pressure and the crude product was purified by silica gel column chromatography (PE/EA (0% to 5% gradient in 20 min)) to afford tert-butyl 2-[3-(bromomethyl)phenyl]-2-oxoacetate (4.00 g, 46.6%) as a yellow oil. ¹H NMR (300 MHz, DMSO-d₆) δ 8.01-7.96 (m, 1H), 7.88-7.81 (m, 2H), 7.67-7.56 (m, 1H), 4.84 (s, 2H), 1.59 (s, 9H).

### Step 3: Preparation of tert-butyl 2-oxo-2-{3-[(4,4,5,5-tetramethyl-1,3,2-dioxaborolan-2-yl)methyl]phenyl}acetate

To a stirred solution of tert-butyl 2-[3-(bromomethyl)phenyl]-2-oxoacetate (8.00 g, 26.74 mmol, 1.00 equiv.) and 4,4,5,5-tetramethyl-2-(4,4,5,5-tetramethyl-1,3,2-dioxaborolan-2-yl)-1,3,2-dioxaborolane (8.15 g, 32.08 mmol, 1.20 equiv.) in dioxane (80 mL) was added KOAc (7.87 g, 80.22 mmol, 3.00 equiv.) and Pd(dppf)Cl₂—CH₂Cl₂ (2.18 g, 2.67 mmol, 0.10 equiv.) at room temperature under nitrogen atmosphere. The resulting mixture was then heated to 100° C. under nitrogen atmosphere. After 3 h, the reaction was cooled to room temperature, and concentrated under reduced pressure. The residue was purified by silica gel column chromatography (PE/EA (0% to 10% gradient in 30 min)) to afford tert-butyl 2-oxo-2-{3-[(4,4,5,5-tetramethyl-1,3,2-dioxaborolan-2-yl)methyl]phenyl}acetate (6.00 g, 61.6%) as a yellow oil. ¹H NMR (300 MHz, DMSO-d₆) δ 7.69-7.63 (m, 2H), 7.56-7.44 (m, 2H), 2.35 (s, 2H), 1.59 (s, 9H), 1.17 (s, 12H).

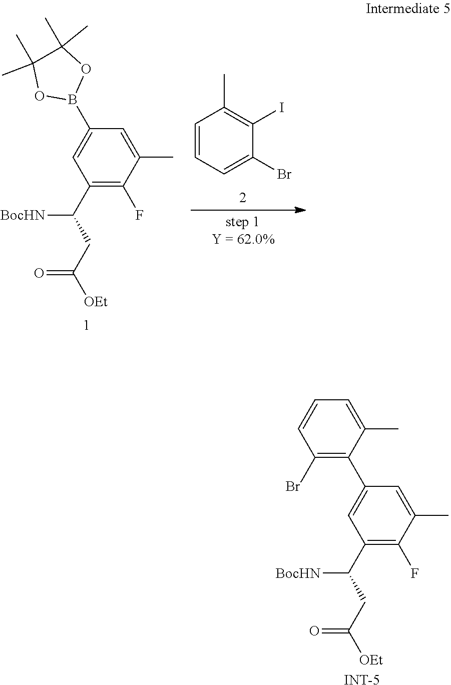

### Step 1: Preparation of ethyl (3S)-3-{2′-bromo-4-fluoro-5,6′-dimethyl-[1,1′-biphenyl]-3-yl}-3-[(tert-butoxycarbonyl)amino]propanoate

To a stirred mixture of ethyl (3S)-3-[(tert-butoxycarbonyl)amino]-3-[2-fluoro-3-methyl-5-(4,4,5,5-tetramethyl-1,3,2-dioxaborolan-2-yl)phenyl]propanoate (2.00 g, 4.43 mmol, 1.00 equiv) and 1-bromo-2-iodo-3-methylbenzene (1.45 g, 4.87 mmol, 1.10 equiv) in dioxane (20 mL) and H₂O (2 mL) were added K₂CO₃ (1.84 g, 13.29 mmol, 3.00 equiv) and Pd(dppf)Cl₂ (0.32 g, 0.44 mmol, 0.10 equiv) at room temperature under nitrogen atmosphere. The resulting mixture was stirred for 2 h at 100° C. under nitrogen atmosphere. The reaction was monitored by LCMS. The mixture was allowed to cool down to room temperature and then concentrated. The crude product was purified by silica gel column chromatography (PE/EA (0% to 20% gradient in 25 min)) to afford ethyl (3S)-3-{2′-bromo-4-fluoro-5,6′-dimethyl-[1,1′-biphenyl]-3-yl}-3-[(tert-butoxycarbonyl)amino]propanoate (1.4 g, 63.9%) as a light yellow oil. LC-MS: (ES+H, m/z): [M+H]⁺=494.1; ¹H NMR (400 MHz, DMSO-d₆) δ 7.57-7.47 (m, 2H), 7.35-7.29 (m, 1H), 7.21 (t, J=7.6 Hz, 1H), 7.08-6.96 (m, 2H), 5.28 (h, J=8.2, 7.3 Hz, 1H), 4.07-3.99 (m, 2H), 2.67 (t, J=7.5 Hz, 2H), 2.26 (t, J=1.3 Hz, 3H), 2.01 (d, J=8.5 Hz, 3H), 1.33 (d, J=2.9 Hz, 9H), 1.12 (td, J=7.1, 4.5 Hz, 3H). ¹⁹F NMR (377 MHz, DMSO-d₆) δ −125.96.

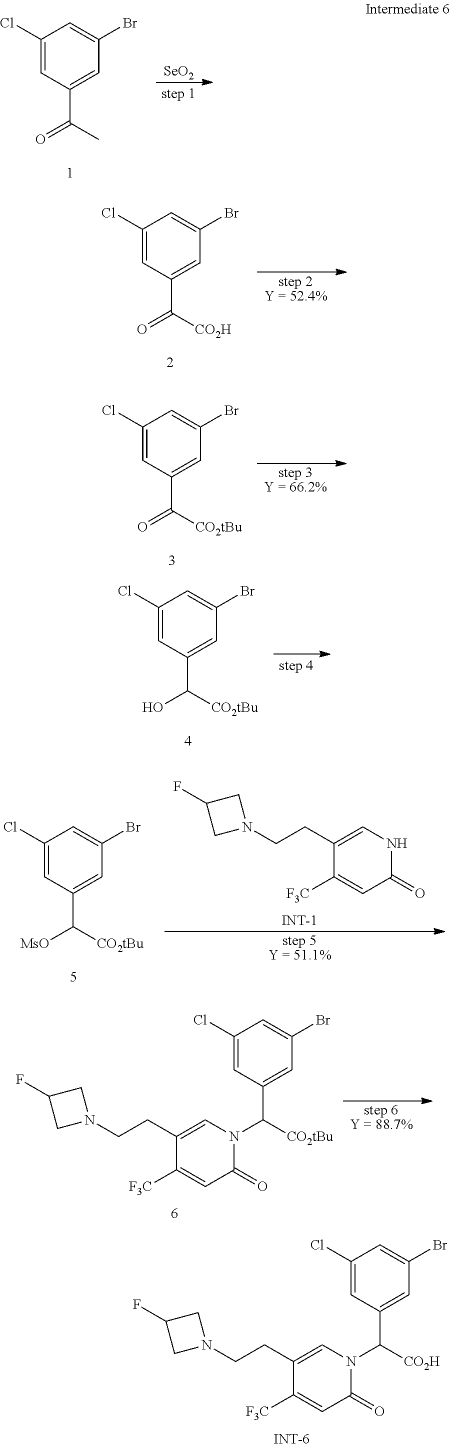

### Step 1: Preparation of (3-bromo-5-chlorophenyl)(oxo)acetic acid

To a stirred mixture of 1-(3-bromo-5-chlorophenyl)ethanone (10.00 g, 42.82 mmol, 1.00 equiv) in pyridine (100 mL) was added SeO₂ (7.13 g, 64.24 mmol, 1.50 equiv) at room temperature under nitrogen atmosphere. The resulting mixture was stirred for 2 h at 100° C. under nitrogen atmosphere. The reaction was monitored by LCMS. The mixture was then cooled to room temperature and acidified to pH 5 with conc. HCl. The resulting mixture was filtered and the filter cake was washed with EtOAc (3×300 mL). The combined organic layers were washed with brine (1×500 mL), dried over anhydrous Na₂SO₄, filtered and concentrated to afford (3-bromo-5-chlorophenyl)(oxo)acetic acid (12 g, crude) as a yellow solid. LC-MS: (ES−H, m/z): [M−H]⁻=260.8; ¹H NMR (400 MHz, DMSO-d₆) δ 8.15 (t, J=1.9 Hz, 1H), 8.05 (t, J=1.6 Hz, 1H), 7.97 (t, J=1.7 Hz, 1H).

### Step 2: Preparation of tert-butyl 2-(3-bromo-5-chlorophenyl)-2-oxoacetate

To a stirred mixture of (3-bromo-5-chlorophenyl)(oxo)acetic acid (11.00 g, 41.75 mmol, 1.00 equiv) in DCM (200 mL) was added (Z)—N,N′-diisopropyltert-butoxymethanimidamide (16.73 g, 83.50 mmol, 2.00 equiv) at room temperature under nitrogen atmosphere. The resulting mixture was stirred overnight at room temperature under nitrogen atmosphere. The reaction was monitored by LCMS. The resulting mixture was filtered and the filter cake was washed with EtOAc (3×500 mL). The filtrate was concentrated under reduced pressure and the crude product was purified by silica gel column chromatography (PE/CH₂Cl₂ (0% to 10% gradient in 30 min)) to afford tert-butyl 2-(3-bromo-5-chlorophenyl)-2-oxoacetate (7 g, 52.4%) as a yellow oil. ¹H NMR (400 MHz, DMSO-d₆) δ 8.21 (dq, J=2.4, 1.5 Hz, 1H), 8.06 (q, J=1.7 Hz, 1H), 7.97 (q, J=1.8 Hz, 1H), 1.63 (s, 9H).

### Step 3: Preparation of tert-butyl 2-(3-bromo-5-chlorophenyl)-2-hydroxyacetate

To a stirred mixture of tert-butyl 2-(3-bromo-5-chlorophenyl)-2-oxoacetate (2.40 g, 7.51 mmol, 1.00 equiv) and CH₃CO₂H (1.5 mL) in CH₃CH₂OH (15 mL) was added NaBH₃CN (519 mg, 8.26 mmol, 1.10 equiv) at room temperature under nitrogen atmosphere. The resulting mixture was stirred for 1 h at room temperature under nitrogen atmosphere. The reaction was monitored by LCMS. The reaction was quenched with water (200 mL) at room temperature. The resulting mixture was extracted with EtOAc (3×250 mL). The combined organic layers were washed with brine (1×200 mL), dried over anhydrous Na₂SO₄, filtered and concentrated. The residue was purified by silica gel column chromatography, eluted with PE/CH₂Cl₂ (0% to 50% gradient in 30 min) to afford tert-butyl 2-(3-bromo-5-chlorophenyl)-2-hydroxyacetate (1.6 g, 66.2%) as a white solid. LC-MS: (ES−H, m/z): [M−H]⁻=318.9; ¹H NMR (300 MHz, DMSO-d₆) δ 7.68 (t, J=1.9 Hz, 1H), 7.57-7.52 (m, 1H), 7.46 (ddd, J=2.0, 1.4, 0.6 Hz, 1H), 6.25 (d, J=5.8 Hz, 1H), 5.08 (d, J=5.8 Hz, 1H), 1.36 (s, 9H).

### Step 4: Preparation of tert-butyl 2-(3-bromo-5-chlorophenyl)-2-(methanesulfonyloxy)acetate

To a stirred mixture of tert-butyl 2-(3-bromo-5-chlorophenyl)-2-hydroxyacetate (1.60 g, 4.97 mmol, 1.00 equiv) and NEt₃ (755 mg, 7.46 mmol, 1.50 equiv) in DCM (15 mL) was added MsCl (684 mg, 5.97 mmol, 1.20 equiv) dropwise at 0° C. under nitrogen atmosphere. The resulting mixture was stirred for 1 h at room temperature under nitrogen atmosphere. The reaction was monitored by LCMS. The reaction was quenched with water at room temperature. The resulting mixture was extracted with EtOAc (3×500 mL). The combined organic layers were washed with brine (1×400 mL), dried over anhydrous Na₂SO₄, filtered and concentrated to afford tert-butyl 2-(3-bromo-5-chlorophenyl)-2-(methanesulfonyloxy)acetate (2 g, crude) as a white solid. LC-MS: (ES−H, m/z): [M−H]⁻=396.9;
Step 5: Preparation of tert-butyl 2-(3-bromo-5-chlorophenyl)-2-{5-[2-(3-fluoroazetidin-1-yl)ethyl]-2-oxo-4-(trifluoromethyl)pyridin-1-yl}acetate

To a stirred mixture of tert-butyl 2-(3-bromo-5-chlorophenyl)-2-(methanesulfonyloxy)acetate (1.82 g, 4.54 mmol, 1.20 equiv) and 5-[2-(3-fluoroazetidin-1-yl)ethyl]-4-(trifluoromethyl)-1H-pyridin-2-one (1.00 g, 3.78 mmol, 1.00 equiv) in MeCN (10 mL) was added K₂CO₃ (1.05 g, 7.57 mmol, 2.00 equiv) at room temperature under nitrogen atmosphere. The resulting mixture was stirred overnight at 80° C. under nitrogen atmosphere. The resulting mixture was filtered, and the filter cake was washed with EtOAc (3×500 mL). The filtrate was concentrated under reduced pressure. The residue was purified by reversed-phase flash chromatography (C18; mobile phase, MeCN in water (0.1% HCO₂H), 0% to 30% gradient in 10 min; detector, UV 220 nm) to afford tert-butyl 2-(3-bromo-5-chlorophenyl)-2-{5-[2-(3-fluoroazetidin-1-yl)ethyl]-2-oxo-4-(trifluoromethyl)pyridin-1-yl}acetate (1.1 g, 51.1%) as a colorless oil. LC-MS: (ES+H, m/z): [M+H]⁺=567.1.

### Step 6: Preparation of (3-bromo-5-chlorophenyl)({5-[2-(3-fluoroazetidin-1-yl)ethyl]-2-oxo-4-(trifluoromethyl)pyridin-1-yl})acetic acid

To a stirred mixture of tert-butyl 2-(3-bromo-5-chlorophenyl)-2-{5-[2-(3-fluoroazetidin-1-yl)ethyl]-2-oxo-4-(trifluoromethyl)pyridin-1-yl}acetate (1.00 g, 1.76 mmol, 1.00 equiv) in DCM (4 mL) was added TFA (4 mL) at room temperature under nitrogen atmosphere. The resulting mixture was stirred for 1 h at room temperature under nitrogen atmosphere. The reaction was monitored by LCMS. The resulting mixture was concentrated under reduced pressure. The residue was purified by reversed-phase flash chromatography (C18; mobile phase, MeCN in water (0.1% NH₃H₂O), 0% to 20% gradient in 30 min; detector, UV 220 nm) to afford (3-bromo-5-chlorophenyl)({5-[2-(3-fluoroazetidin-1-yl)ethyl]-2-oxo-4-(trifluoromethyl)pyridin-1-yl})acetic acid (800 mg, 88.7%) as a white solid. LC-MS: (ES+H, m/z): [M+H]⁺=511.0; ¹H NMR (300 MHz, DMSO-d₆) δ 7.85 (s, 1H), 7.75 (t, J=1.8 Hz, 1H), 7.65 (t, J=1.6 Hz, 1H), 7.56 (q, J=2.1, 1.7 Hz, 1H), 6.82 (d, J=5.2 Hz, 1H), 6.30 (s, 1H), 5.31-5.04 (m, 2H), 3.46 (d, J=23.4 Hz, 4H), 2.82 (d, J=7.3 Hz, 2H), 2.55 (t, J=3.5 Hz, 2H). ¹⁹F NMR (282 MHz, DMSO-d₆) δ −62.20, −177.84.

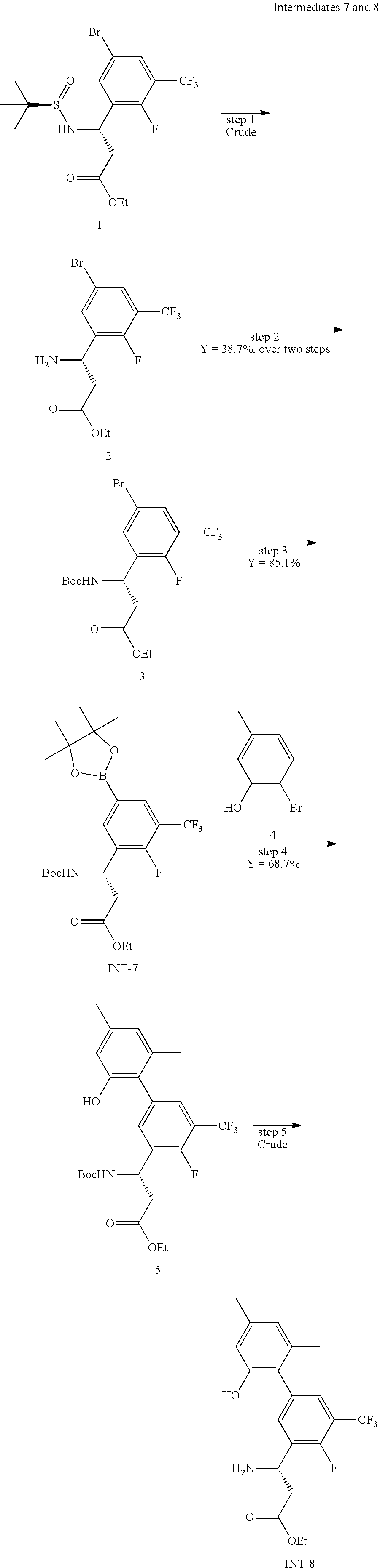

### Step 1: Preparation of ethyl (3S)-3-amino-3-[5-bromo-2-fluoro-3-(trifluoromethyl)phenyl]propanoate hydrochloride

To a stirred solution of ethyl (3S)-3-[5-bromo-2-fluoro-3-(trifluoromethyl)phenyl]-3-{[(S)-2-methylpropane-2-sulfinyl]amino}propanoate (prepared according to WO2021076890) (10.00 g, 21.63 mmol, 1.00 equiv) in DCM (15 mL) were added HCl (gas) in 1,4-dioxane (30 mL, 4 M) dropwise at 0° C. under nitrogen atmosphere. The resulting mixture was stirred for an additional 1.5 h at room temperature. The reaction was monitored by LCMS. The resulting mixture was concentrated under reduced pressure to provide crude ethyl (3S)-3-amino-3-[5-bromo-2-fluoro-3-(trifluoromethyl)phenyl]propanoate hydrochloride (11.50 g) as a yellow oil. The crude product mixture was used in the next step directly without further purification. LC-MS: (ES+H, m/z): [M+H]⁺=359.9.

### Step 2: Preparation of ethyl (3S)-3-[5-bromo-2-fluoro-3-(trifluoromethyl)phenyl]-3-[(tert-butoxycarbonyl)amino]propanoate

To a stirred solution of ethyl (3S)-3-amino-3-[5-bromo-2-fluoro-3-(trifluoromethyl)phenyl]propanoate hydrochloride (11.50 g, assumed 100% yield, 32.11 mmol, 1.00 equiv) in DCM (100 mL) was added DIEA (16.64 g, 128.76 mmol, 4.01 equiv) and Boc₂O (14.02 g, 64.22 mmol, 2.00 equiv) dropwise at 0° C. under nitrogen atmosphere. The resulting mixture was stirred for an additional 2 h at room temperature. The reaction was monitored by LCMS. The resulting mixture was diluted with water (100 mL) and extracted with CH₂Cl₂ (3×120 mL). The combined organic layers were washed with brine (2×40 mL), dried over anhydrous Na₂SO₄, filtered and concentrated. The crude product was purified by silica gel column chromatography (PE/EA (5% to 25% gradient in 20 min)) to afford ethyl (3S)-3-[5-bromo-2-fluoro-3-(trifluoromethyl)phenyl]-3-[(tert-butoxycarbonyl)amino]propanoate (3.80 g, 38.7%, over 2 steps) as a white solid. LC-MS: (ES+H, m/z): [M+H−tBu]⁺=404.1; ¹H NMR (300 MHz, CDCl₃): δ 7.69 (dd, J=6.1, 2.5 Hz, 1H), 7.64 (dd, J=6.0, 2.5 Hz, 1H), 5.76 (s, 1H), 5.30 (s, 1H), 4.10 (q, J=7.1 Hz, 2H), 2.85 (d, J=5.8 Hz, 2H), 1.43 (s, 9H), 1.19 (t, J=7.1 Hz, 3H). ¹⁹F NMR (282 MHz, CDCl₃) δ −61.41, −121.99.

### Step 3: Preparation of ethyl (3S)-3-[(tert-butoxycarbonyl)amino]-3-[2-fluoro-5-(4,4,5,5-tetramethyl-1,3,2-dioxaborolan-2-yl)-3-(trifluoromethyl)phenyl]propanoate

To a stirred solution of ethyl (3S)-3-[5-bromo-2-fluoro-3-(trifluoromethyl)phenyl]-3-[(tert-butoxycarbonyl)amino]propanoate (6.60 g, 14.40 mmol, 1.00 equiv) and 4,4,5,5-tetramethyl-2-(4,4,5,5-tetramethyl-1,3,2-dioxaborolan-2-yl)-1,3,2-dioxaborolane (3.66 g, 14.40 mmol, 1.00 equiv) in 1,4-dioxane (80 mL) were added KOAc (4.24 g, 43.20 mmol, 3.00 equiv) and Pd(dppf)Cl₂·CH₂Cl₂ (1.17 g, 1.44 mmol, 0.10 equiv) at room temperature under nitrogen atmosphere. The resulting mixture was stirred for an additional 2 h at 110° C. The mixture was allowed to cool down to room temperature. The resulting mixture was filtered, and the filter cake was washed with EtOAc (3×30 mL). The filtrate was concentrated under reduced pressure. The residue was purified by silica gel column chromatography, eluted with PE/EA (10% to 30% gradient in 20 min) to afford ethyl (3S)-3-[(tert-butoxycarbonyl)amino]-3-[2-fluoro-5-(4,4,5,5-tetramethyl-1,3,2-dioxaborolan-2-yl)-3-(trifluoromethyl)phenyl]propanoate (6.2 g, 85.1%) as yellow oil. LC-MS: (ES+H, m/z): [M+Na]⁺=528.4; ¹H NMR (400 MHz, CDCl₃): δ 7.96 (d, J=3.1 Hz, 1H), 7.95 (d, J=3.2 Hz, 1H), 5.58 (s, 1H), 5.37 (s, 1H), 4.08 (q, J=7.1 Hz, 2H), 2.83 (q, J=8.9, 8.3 Hz, 2H), 1.43 (s, 9H), 1.34 (d, J=1.6 Hz, 12H), 1.18 (t, J=7.1 Hz, 3H). ¹⁹F NMR (377 MHz, CDCl₃) δ −61.09, −116.29.

### Step 4: Preparation of ethyl (3S)-3-[(tert-butoxycarbonyl)amino]-3-[4-fluoro-2′-hydroxy-4′,6′-dimethyl-5-(trifluoromethyl)-[1,1′-biphenyl]-3-yl]propanoate

To a stirred solution of ethyl (3S)-3-[(tert-butoxycarbonyl)amino]-3-[2-fluoro-5-(4,4,5,5-tetramethyl-1,3,2-dioxaborolan-2-yl)-3-(trifluoromethyl)phenyl]propanoate (750 mg, 1.48 mmol, 1.00 equiv) and 2-bromo-3,5-dimethylphenol (298 mg, 1.48 mmol, 1.00 equiv) in 1,4-dioxane (10 mL) and H₂O (0.5 mL) were added Pd(dppf)Cl₂·CH₂Cl₂ (120 mg, 0.14 mmol, 0.10 equiv) and K₂CO₃ (615. mg, 4.45 mmol, 3.00 equiv) at room temperature under nitrogen atmosphere. The resulting mixture was stirred for additional 2 h at 100° C. The reaction was monitored by LCMS. The mixture was allowed to cool down to room temperature. The resulting mixture was diluted with water (30 mL) and extracted with EtOAc (3×50 mL). The combined organic layers were washed with brine (1×8 mL), dried over anhydrous Na₂SO₄, filtered and concentrated. The crude product was purified by silica gel column chromatography (PE/EA (10% to 40% gradient in 10 min)) to afford ethyl (3S)-3-[(tert-butoxycarbonyl)amino]-3-[4-fluoro-2′-hydroxy-4′,6′-dimethyl-5-(trifluoromethyl)-[1,1′-biphenyl]-3-yl]propanoate (510 mg, 68.7%) as light yellow oil. LC-MS: (ES+H, m/z): [M+H]⁺=500.2; ¹H NMR (400 MHz, CDCl₃) δ 7.50 (d, J=6.4 Hz, 1H), 7.44 (d, J=6.3 Hz, 1H), 6.69 (s, 1H), 6.65 (s, 1H), 5.79 (s, 1H), 5.39 (s, 1H), 4.65 (s, 1H), 4.07 (dd, J=12.9, 5.6 Hz, 2H), 2.90 (s, 2H), 2.31 (s, 3H), 2.00 (s, 3H), 1.41 (s, 9H), 1.18 (t, J=6.9 Hz, 3H).

### Step 5: Preparation of ethyl (3S)-3-amino-3-[4-fluoro-2′-hydroxy-4′,6′-dimethyl-5-(trifluoromethyl)-[1,1′-biphenyl]-3-yl]propanoate hydrochloride

To a stirred solution of ethyl (3S)-3-[(tert-butoxycarbonyl)amino]-3-[4-fluoro-2′-hydroxy-4′,6′-dimethyl-5-(trifluoromethyl)-[1,1′-biphenyl]-3-yl]propanoate (480 mg, 0.96 mmol, 1.00 equiv) in DCM (7 mL) was added HCl (gas) in 1,4-dioxane (5 mL, 4 M) dropwise at 0° C. under nitrogen atmosphere. The resulting mixture was stirred for an additional 1 h at room temperature. The reaction was monitored by LCMS. Desired product could be detected by LCMS. The resulting mixture was concentrated under reduced pressure to afford ethyl (3S)-3-amino-3-[4-fluoro-2′-hydroxy-4′,6′-dimethyl-5-(trifluoromethyl)-[1,1′-biphenyl]-3-yl]propanoate hydrochloride (510 mg, Crude) as a yellow solid. LC-MS: (ES+H, m/z): [M+H]⁺=400.1.

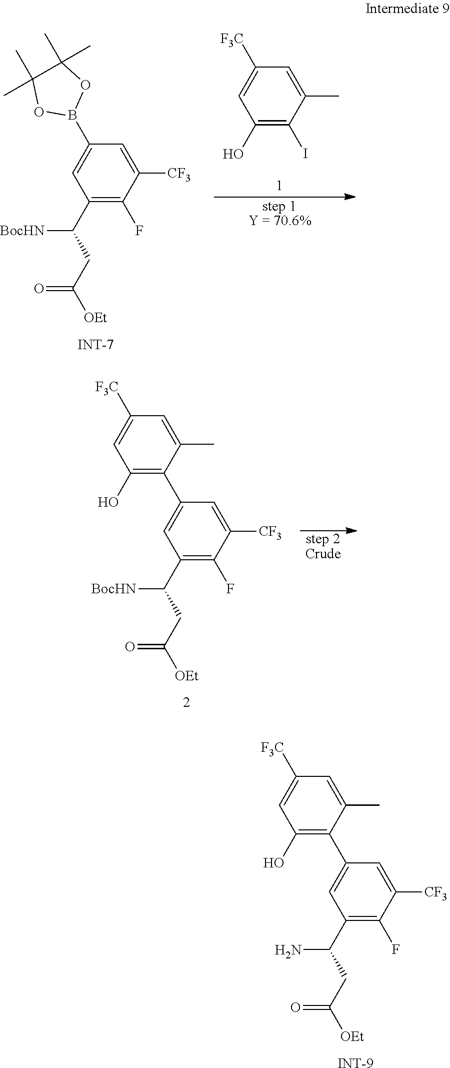

### Step 1: Preparation of ethyl (3S)-3-[(tert-butoxycarbonyl)amino]-3-[4-fluoro-2′-hydroxy-6′-methyl-4′,5-bis(trifluoromethyl)-[1,1′-biphenyl]-3-yl]propanoate

A mixture of ethyl (3S)-3-[(tert-butoxycarbonyl)amino]-3-[2-fluoro-5-(4,4,5,5-tetramethyl-1,3,2-dioxaborolan-2-yl)-3-(trifluoromethyl)phenyl]propanoate (750 mg, 1.48 mmol, 1.00 equiv), 2-iodo-3-methyl-5-(trifluoromethyl)phenol (471 mg, 1.56 mmol, 1.05 equiv), Pd(dppf)Cl₂ (109 mg, 0.15 mmol, 0.10 equiv) and K₂CO₃ (410 mg, 2.97 mmol, 2.00 equiv) in dioxane (10 mL) and H₂O (0.5 mL) was stirred for 2 h at 100° C. under nitrogen atmosphere and then cooled to room temperature. The residue was purified by silica gel column chromatography (PE/EA (0% to 30% in 20 min)) to afford ethyl (3S)-3-[(tert-butoxycarbonyl)amino]-3-[4-fluoro-2′-hydroxy-6′-methyl-4′,5-bis(trifluoromethyl)-[1,1′-biphenyl]-3-yl]propanoate (580 mg, 70.6%) as an off-white solid. LC-MS: (ES+H, m/z): [M+H]⁺=554.25; ¹H NMR (300 MHz, CDCl₃) δ 7.51 (d, J=6.4 Hz, 1H), 7.45 (dd, J=6.5, 2.1 Hz, 1H), 7.10 (d, J=7.9 Hz, 2H), 5.84 (s, 1H), 5.39 (d, J=6.8 Hz, 1H), 4.19-4.06 (m, 2H), 2.94 (d, J=5.7 Hz, 2H), 2.09 (s, 3H), 1.43 (s, 9H), 1.21 (t, J=7.1 Hz, 3H). ¹⁹F NMR (282 MHz, CDCl₃) δ −61.14, −61.19, −62.99, −120.65.

### Step 2: Preparation of ethyl (3S)-3-amino-3-[4-fluoro-2′-hydroxy-6′-methyl-4′,5-bis(trifluoromethyl)-[1,1′-biphenyl]-3-yl]propanoate

A solution of ethyl (3S)-3-[(tert-butoxycarbonyl)amino]-3-[4-fluoro-2′-hydroxy-6′-methyl-4′,5-bis(trifluoromethyl)-[1,1′-biphenyl]-3-yl]propanoate (500 mg, 0.90 mmol, 1.00 equiv) and HCl (gas) in 1,4-dioxane (4 M, 5 mL) in DCM (5 mL) was stirred for 2 h at room temperature under nitrogen atmosphere. The reaction was monitored by LCMS. The resulting mixture was concentrated under reduced pressure to afford ethyl (3S)-3-amino-3-[4-fluoro-2′-hydroxy-6′-methyl-4′,5-bis(trifluoromethyl)-[1,1′-biphenyl]-3-yl]propanoate (500 mg, crude) as a colorless oil. The crude product was used in the next step directly without further purification. LC-MS: (ES+H, m/z): [M+H]⁺=454.10.

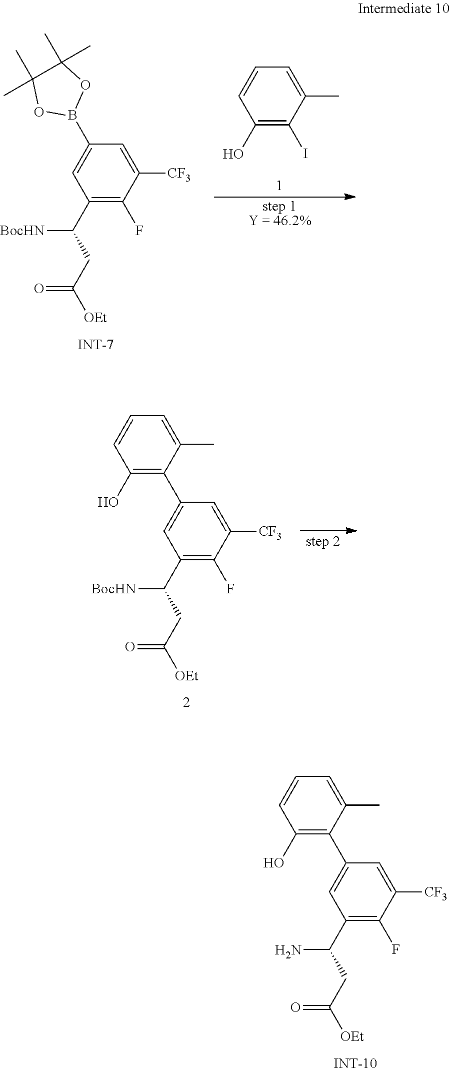

### Step 1: Preparation of ethyl (3S)-3-[(tert-butoxycarbonyl)amino]-3-[4-fluoro-2′-hydroxy-6′-methyl-5-(trifluoromethyl)-[1,1′-biphenyl]-3-yl]propanoate

To a stirred solution of ethyl (3S)-3-[(tert-butoxycarbonyl)amino]-3-[2-fluoro-5-(4,4,5,5-tetramethyl-1,3,2-dioxaborolan-2-yl)-3-(trifluoromethyl)phenyl]propanoate (1.80 g, 3.56 mmol, 1.00 equiv) and 2-bromo-3-methylphenol (0.80 g, 4.27 mmol, 1.20 equiv) in dioxane (50 mL) were added Pd(PPh₃)₄ (0.41 g, 0.36 mmol, 0.10 equiv), K₂HPO₄ (1.86 g, 10.68 mmol, 3.00 equiv) in H₂O (5 mL) dropwise at room temperature under nitrogen atmosphere. The resulting mixture was stirred overnight at 90° C. under nitrogen atmosphere. The reaction was monitored by LCMS. The mixture was allowed to cool down to room temperature. The resulting mixture was diluted with EtOAc (200 mL) and washed with water (2×30 mL). The combined organic layers were washed with brine (1×30 mL), dried over anhydrous Na₂SO₄, filtered and concentrated. The residue was purified by reversed-phase flash chromatography (C18; mobile phase, MeCN in water (0.1% NH₃·H₂O), 0% to 70% gradient in 40 min; detector, UV 220 nm) to afford ethyl (3S)-3-[(tert-butoxycarbonyl)amino]-3-[4-fluoro-2′-hydroxy-6′-methyl-5-(trifluoromethyl)-[1,1′-biphenyl]-3-yl]propanoate (0.8 g, 46.2%) as a brown solid. LC-MS: (ES−H, m/z): [M−H]⁻=484.1; ¹H NMR (300 MHz, DMSO-d6) δ 9.55 (s, 1H), 7.77-7.53 (m, 2H), 7.43 (dd, J=6.6, 2.1 Hz, 1H), 7.09 (t, J=7.8 Hz, 1H), 6.87 (d, J=8.1 Hz, 1H), 6.75 (d, J=7.5 Hz, 1H), 5.33 (q, J=7.9 Hz, 1H), 4.08-3.97 (m, 2H), 2.86-2.65 (m, 2H), 1.98 (s, 3H), 1.34 (s, 9H), 1.11 (t, J=7.1 Hz, 3H).

### Step 2: Preparation of ethyl (3S)-3-amino-3-[4-fluoro-2′-hydroxy-6′-methyl-5-(trifluoromethyl)-[1,1′-biphenyl]-3-yl]propanoate

To a stirred solution of ethyl (3S)-3-[(tert-butoxycarbonyl)amino]-3-[4-fluoro-2′-hydroxy-6′-methyl-5-(trifluoromethyl)-[1,1′-biphenyl]-3-yl]propanoate (0.80 g, 1.65 mmol, 1.00 equiv) in CH₂Cl₂ (4 mL) was added HCl (gas) in 1,4-dioxane (8 mL, 4 M) dropwise at room temperature under nitrogen atmosphere. The resulting mixture was stirred for 3 h at room temperature under nitrogen atmosphere. The reaction was monitored by LCMS. The resulting mixture was concentrated under vacuum. The crude product ethyl (3S)-3-amino-3-[4-fluoro-2′-hydroxy-6′-methyl-5-(trifluoromethyl)-[1,1′-biphenyl]-3-yl]propanoate (800 mg, crude) was used in the next step directly without further purification. LC-MS: (ES+H, m/z): [M+H]⁺=386.1.

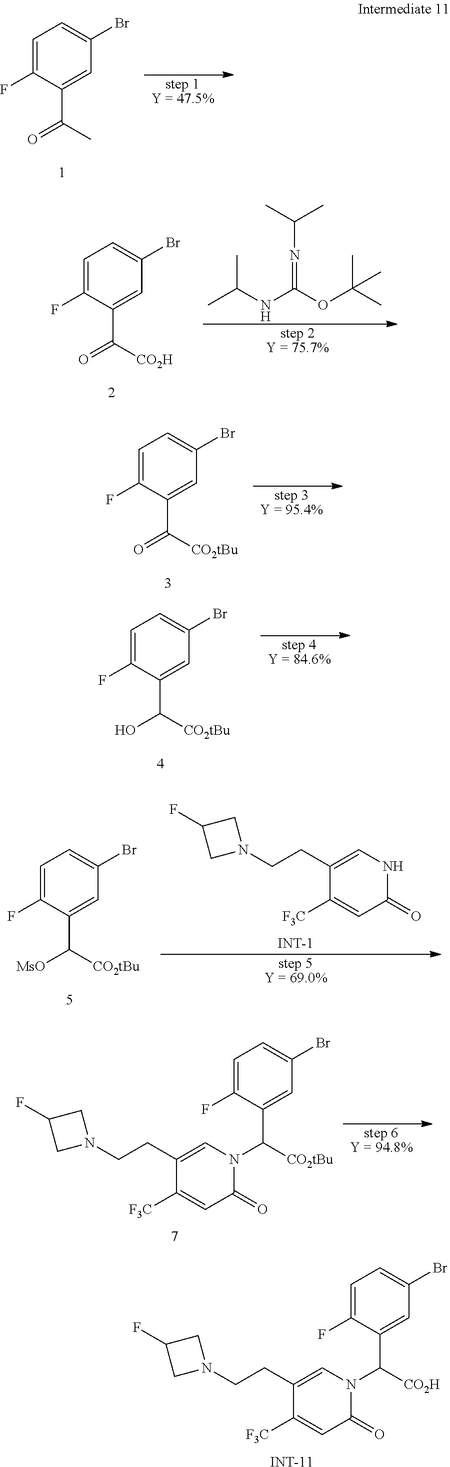

### Step 1: Preparation of (5-bromo-2-fluorophenyl)(oxo)acetic acid

A mixture of 1-(5-bromo-2-fluorophenyl)ethanone (10.00 g, 46.07 mmol, 1.00 equiv) and SeO₂ (10.23 g, 92.15 mmol, 2.00 equiv) in pyridine (20 mL) was stirred for 2 h at 100° C. under nitrogen atmosphere. The reaction was monitored by TLC (CH₂Cl₂:MeOH=10:1). The resulting mixture was diluted with 1 N HCl (aq.) (200 mL) and extracted with EtOAc (3×200 mL). The combined organic layers were concentrated under reduced pressure and the crude product was purified by silica gel column chromatography (CH₂Cl₂/MeOH (0% to 10% gradient in 20 min)) to afford (5-bromo-2-fluorophenyl)(oxo)acetic acid (5.4 g, 47.5%) as a yellow oil. ¹H NMR (300 MHz, DMSO-d₆) δ 7.95-7.80 (m, 2H), 7.35 (t, J=9.3 Hz, 1H).

### Step 2: Preparation of tert-butyl 2-(5-bromo-2-fluorophenyl)-2-oxoacetate

To a stirred solution of (5-bromo-2-fluorophenyl)(oxo)acetic acid (1.40 g, 5.66 mmol, 1.00 equiv) in tetrahydrofuran (10 mL) was added N,N′-diisopropyltert-butoxymethanimidamide (3.97 g, 19.83 mmol, 3.50 equiv) dropwise at 0° C. under nitrogen atmosphere. The resulting mixture was stirred for 3 h at 0° C. under nitrogen atmosphere. The reaction was monitored by TLC (PE/EtOAc=5/1). The resulting mixture was concentrated under reduced pressure. The residue was purified by silica gel column chromatography, eluted with PE/EtOAc (0% to 20% gradient in 30 min) to afford tert-butyl 2-(5-bromo-2-fluorophenyl)-2-oxoacetate (1.3 g, 75.7%) as a colorless oil. ¹H NMR (300 MHz, DMSO-d₆) δ 8.08-7.94 (m, 2H), 7.48 (dd, J=10.6, 8.8 Hz, 1H), 1.54 (s, 9H).

### Step 3: Preparation of tert-butyl 2-(5-bromo-2-fluorophenyl)-2-hydroxyacetate

A mixture of tert-butyl 2-(5-bromo-2-fluorophenyl)-2-oxoacetate (900 mg, 2.96 mmol, 1.00 equiv) and NaBH₃CN (205 mg, 3.26 mmol, 1.10 equiv) in (EtOH:HCOOH=8:1) was stirred for 1 h at room temperature under nitrogen atmosphere. The reaction was monitored by TLC (PE/EtOAc=5/1). The resulting mixture was diluted with water (20 mL). The resulting mixture was extracted with EtOAc (3×50 mL). The combined organic layers were washed with water (1×50 mL), dried over anhydrous Na₂SO₄. After filtration, the filtrate was concentrated under reduced pressure to provide tert-butyl 2-(5-bromo-2-fluorophenyl)-2-hydroxyacetate (864 mg, 95.4%) as a yellow oil. ¹H NMR (300 MHz, DMSO-d₆) δ 7.61 (dd, J=6.4, 2.6 Hz, 1H), 7.56 (ddd, J=8.7, 4.6, 2.6 Hz, 1H), 7.31-7.15 (m, 1H), 5.18 (s, 1H), 1.35 (s, 9H).

### Step 5: Preparation of tert-butyl 2-(5-bromo-2-fluorophenyl)-2-{5-[2-(3-fluoroazetidin-1-yl)ethyl]-2-oxo-4-(trifluoromethyl)pyridin-1-yl}acetate

To a stirred mixture of tert-butyl 2-(5-bromo-2-fluorophenyl)-2-(methanesulfonyloxy)acetate (942 mg, 2.46 mmol, 1.30 equiv) and 5-[2-(3-fluoroazetidin-1-yl)ethyl]-4-(trifluoromethyl)-1H-pyridin-2-one (500 mg, 1.89 mmol, 1.00 equiv) in CH₃CN (10 mL) was added K₂CO₃ (784 mg, 5.67 mmol, 3.00 equiv) in portions at room temperature under nitrogen atmosphere. The resulting mixture was stirred for 6 h at 80° C. under nitrogen atmosphere. The reaction was monitored by LCMS. The mixture was allowed to cool down to room temperature and concentrated. The residue was purified by silica gel column chromatography (PE/EtOAc (0% to 30% gradient in 40 min)) to afford tert-butyl 2-(5-bromo-2-fluorophenyl)-2-{5-[2-(3-fluoroazetidin-1-yl)ethyl]-2-oxo-4-(trifluoromethyl)pyridin-1-yl}acetate (720 mg, 69.0%) as a white solid. LC-MS: (ES+H, m/z): [M+H]⁺=551.1; ¹H NMR (300 MHz, DMSO-d₆) δ 7.74 (ddd, J=8.8, 4.6, 2.5 Hz, 1H), 7.68-7.59 (m, 2H), 7.37 (dd, J=10.1, 8.8 Hz, 1H), 6.87 (s, 1H), 6.51 (s, 1H), 5.16-4.89 (m, 1H), 3.44 (ddd, J=15.5, 8.5, 5.9 Hz, 2H), 2.98 (ddt, J=24.1, 8.8, 4.5 Hz, 2H), 2.58-2.38 (m, 4H), 1.41 (s, 9H).

### Step 6: Preparation of (5-bromo-2-fluorophenyl)({5-[2-(3-fluoroazetidin-1-yl)ethyl]-2-oxo-4-(trifluoromethyl)pyridin-1-yl})acetic acid

To a stirred solution of tert-butyl 2-(5-bromo-2-fluorophenyl)-2-{5-[2-(3-fluoroazetidin-1-yl)ethyl]-2-oxo-4-(trifluoromethyl)pyridin-1-yl}acetate (600 mg, 1.08 mmol, 1.00 equiv) in CH₂C₂ (15 mL) was added TFA (15 mL) dropwise at 0° C. under nitrogen atmosphere. The resulting mixture was stirred for 3 h at room temperature under nitrogen atmosphere. The reaction was monitored by LCMS. The resulting mixture was concentrated under reduced pressure. The residue was purified by reversed-phase flash chromatography (C18 gel; mobile phase, MeCN in water (0.1% NH₃·H₂O), 0% to 30% gradient in 25 min; detector, UV 254 nm) to provide (5-bromo-2-fluorophenyl)({5-[2-(3-fluoroazetidin-1-yl)ethyl]-2-oxo-4-(trifluoromethyl)pyridin-1-yl})acetic acid (511 mg, 94.8%) as a white solid. LC-MS: (ES+H, m/z): [M+H]⁺=495.0.

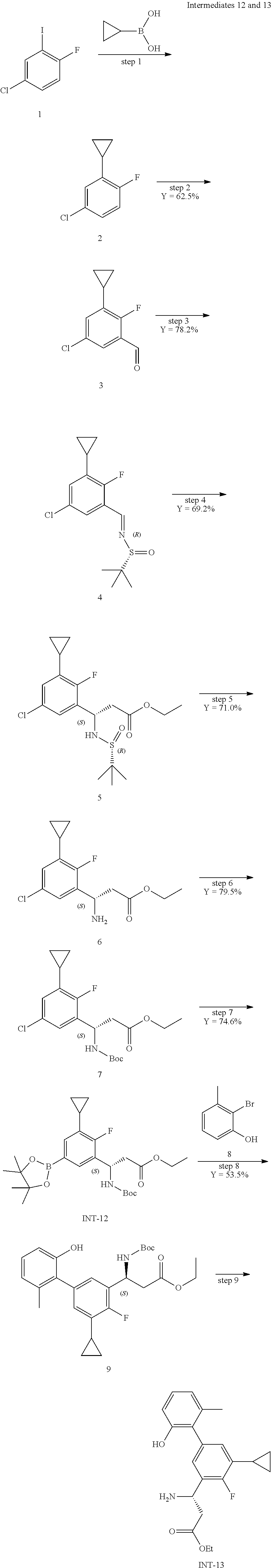

### Step 1: Preparation of 4-chloro-2-cyclopropyl-1-fluorobenzene

To a stirred solution of 4-chloro-1-fluoro-2-iodobenzene (50.00 g, 194.97 mmol, 1.00 equiv) and cyclopropylboronic acid (25.12 g, 292.46 mmol, 1.50 equiv) in PhCH₃ (800 mL) were added Pd(OAc)₂ (4.38 g, 19.49 mmol, 0.10 equiv), tricyclohexylphosphane (10.94 g, 38.99 mmol, 0.20 equiv) and Cs₂CO₃ (127.05 g, 389.95 mmol, 2.00 equiv) in H₂O (80 mL) dropwise at room temperature under nitrogen atmosphere. The resulting mixture was stirred for 6 h at 100° C. under nitrogen atmosphere and then cooled to room temperature. The resulting mixture was diluted with EtOAc (500 mL). The resulting mixture was washed with water (2×100 mL). The combined organic layers were washed with brine (2×50 mL), dried over anhydrous Na₂SO₄, filtered and concentrated. The residue was purified by silica gel column chromatography, eluted with PE/EtOAc (0% to 10% gradient in 30 min) to afford 4-chloro-2-cyclopropyl-1-fluorobenzene (30 g, crude) as a brown yellow oil. ¹H NMR (300 MHz, CDCl₃) δ 7.40-7.06 (m, 1H), 6.95 (dd, J=9.6, 8.7 Hz, 1H), 6.86 (dd, J=6.6, 2.6 Hz, 1H), 2.13-2.03 (m, 1H), 1.07-0.99 (m, 2H), 0.77-0.71 (m, 2H).

### Step 2: Preparation of 5-chloro-3-cyclopropyl-2-fluorobenzaldehyde

To a stirred solution of 4-chloro-2-cyclopropyl-1-fluorobenzene (15.00 g, 87.92 mmol, 1.00 equiv) in THF (150 mL) was added LDA (66 mL, 131.88 mmol, 1.50 equiv, 2 M in THF) dropwise at −78° C. under nitrogen atmosphere. The resulting mixture was stirred for 1 h at −78° C. under nitrogen atmosphere. To the above mixture was added DMF (22.5 mL) dropwise at −78° C. The resulting mixture was stirred for an additional 3 h at −78° C. The reaction was monitored by LCMS. The reaction was quenched by the addition of water (10 mL) at −60° C. The resulting mixture was stirred for 15 mins at −60° C. The resulting mixture was diluted with water (200 mL) and extracted with EtOAc (3×300 mL). The combined organic layers were washed with brine (2×100 mL), dried over anhydrous Na₂SO₄. After filtration, the filtrate was concentrated under reduced pressure. The residue was purified by silica gel column chromatography, eluted with PE/EtOAc (0%-5% gradient in 30 min) to afford 5-chloro-3-cyclopropyl-2-fluorobenzaldehyde (12 g, 65.5%) as a yellow solid. ¹H NMR (300 MHz, CDCl₃) δ 10.34 (s, 1H), 7.63-7.50 (m, 1H), 7.10 (dd, J=6.4, 2.7 Hz, 1H), 2.14-2.12 (m, 1H), 1.15-1.08 (m, 2H), 0.81-0.76 (m, 2H).

### Step 3: Preparation of (S)—N-[(1E)-(5-chloro-3-cyclopropyl-2-fluorophenyl)methylidene]-2-methylpropane-2-sulfinamide

To a stirred solution of 5-chloro-3-cyclopropyl-2-fluorobenzaldehyde (12.00 g, 60.41 mmol, 1.00 equiv) and (R)-2-methylpropane-2-sulfinamide (8.79 g, 72.50 mmol, 1.20 equiv) in THF (150 mL) was added titanium(IV) isopropoxide (25.76 g, 90.62 mmol, 1.50 equiv) dropwise at room temperature under nitrogen atmosphere. The resulting mixture was stirred for 2 h at 50° C. under nitrogen atmosphere. The reaction was monitored by LCMS. The mixture was allowed to cool down to room temperature. The resulting mixture was diluted with EtOAc (20 mL). The reaction was quenched by the addition of water (20 mL) at room temperature. The resulting mixture was stirred for 5 min at room temperature. The precipitated solids were collected by filtration. The resulting mixture was diluted with water (150 mL) and extracted with EtOAc (3×200 mL). The combined organic layers were washed with brine (2×100 mL), dried over anhydrous Na₂SO₄, filtered and concentrated. The crude product was purified by silica gel column chromatography, eluted with PE/EtOAc (0%-10% gradient in 30 min) to afford (S)—N-[(1E)-(5-chloro-3-cyclopropyl-2-fluorophenyl)methylidene]-2-methylpropane-2-sulfinamide (15 g, 78.1%) as a yellow oil. LC-MS: (ES+H, m/z): [M+H]⁺=302.0. ¹H NMR (300 MHz, DMSO-d₆) δ 8.65 (s, 1H), 7.71 (dd, J=5.6, 2.7 Hz, 1H), 7.29 (dd, J=6.5, 2.7 Hz, 1H), 2.15-2.06 (m, 1H), 1.19 (d, J=1.5 Hz, 9H), 1.06-1.02 (m, 2H), 0.87-0.84 (m, 2H).

### Step 4: Preparation of ethyl (3S)-3-(5-chloro-3-cyclopropyl-2-fluorophenyl)-3-{[(R)-2-methylpropane-2-sulfinyl]amino}propanoate

To a stirred solution of Zn (5.80 g, 88.69 mmol, 6.00 equiv) and TMSCl (0.80 g, 7.39 mmol, 0.50 equiv) in THF (50 mL) at room temperature under nitrogen atmosphere. The resulting mixture was stirred for 1 h at 60° C. under nitrogen atmosphere. To the above mixture was added ethyl 2-bromoacetate (7.41 g, 44.34 mmol, 3.00 equiv) dropwise at room temperature. The resulting mixture was stirred for additional 1 h at 60° C. To the above mixture was added (S)—N-[(1E)-(5-chloro-3-cyclopropyl-2-fluorophenyl)methylidene]-2-methylpropane-2-sulfinamide (5.00 g, 16.56 mmol, 1.00 equiv) at 0° C. The resulting mixture was stirred for additional 2 h at room temperature. The reaction was monitored by LCMS. The resulting mixture was diluted with EtOAc (10 mL). The reaction was quenched by the addition of water (10 mL) at room temperature. The resulting mixture was stirred for 5 min at room temperature. The resulting mixture was diluted with water (100 mL). and extracted with EtOAc (3×150 mL). The combined organic layers were washed with brine (2×150 mL), dried over anhydrous Na₂SO₄. After filtration, the filtrate was concentrated under reduced pressure. The residue was purified by silica gel column chromatography, eluted with PE/EtOAc (0%-10% gradient in 30 min) to afford ethyl (3S)-3-(5-chloro-3-cyclopropyl-2-fluorophenyl)-3-{[(R)-2-methylpropane-2-sulfinyl]amino}propanoate (4.2 g, 69.23%) as a yellow oil. LC-MS: (ES+H, m/z): [M+H]⁺=390.1. ¹H NMR (300 MHz, DMSO-d₆) δ 7.32 (dd, J=5.9, 2.6 Hz, 1H), 6.93 (dd, J=6.3, 2.6 Hz, 1H), 5.76 (d, J=7.0 Hz, 1H), 4.95-4.93 (m, 1H), 4.05-4.01 (m, 2H), 2.98 (dd, J=15.5, 7.3 Hz, 1H), 2.83 (dd, J=15.5, 7.4 Hz, 1H), 2.07-1.98 (m, 1H), 1.11 (t, J=7.1 Hz, 3H), 1.05 (s, 9H), 0.98-0.97 (m, 2H), 0.77-0.75 (m, 2H).

### Step 5: Preparation of ethyl (3S)-3-amino-3-(5-chloro-3-cyclopropyl-2-fluorophenyl)propanoate

To a stirred solution of ethyl (3S)-3-(5-chloro-3-cyclopropyl-2-fluorophenyl)-3-{[(S)-2-methylpropane-2-sulfinyl]amino}propanoate (4.20 g, 10.77 mmol, 1.00 equiv) in CH₂Cl₂ (5 mL) was added HCl (gas) in 1,4-dioxane (30 mL, 4 M) dropwise at room temperature. The resulting mixture was stirred for 4 h at room temperature. The reaction was monitored by LCMS. The resulting mixture was concentrated under reduced pressure. The residue was purified by reversed-phase flash chromatography (C18; mobile phase, MeCN in water (0.1% NH₃·H₂O), 10% to 60% gradient in 30 min; detector, UV 254 nm) to afford ethyl (3S)-3-amino-3-(5-chloro-3-cyclopropyl-2-fluorophenyl)propanoate (2.3 g, 71.0%) as a white solid. LC-MS: (ES+H, m/z): [M+H]⁺=286.0. ¹H NMR (300 MHz, DMSO-d₆) δ7.38 (dd, J=6.0, 2.7 Hz, 1H), 6.87 (dd, J=6.3, 2.7 Hz, 1H), 4.45 (dd, J=7.8, 6.2 Hz, 1H), 4.03 (q, J=7.1 Hz, 2H), 2.64-2.54 (m, 3H), 2.07-1.98 (m, 1H), 1.13 (t, J=7.1 Hz, 3H), 0.99-0.96 (m, 2H), 0.76-0.73 (m, 2H).

### Step 6: Preparation of ethyl (3S)-3-[(tert-butoxycarbonyl)amino]-3-(5-chloro-3-cyclopropyl-2-fluorophenyl)propanoate

To a stirred solution of ethyl (3S)-3-amino-3-(5-chloro-3-cyclopropyl-2-fluorophenyl)propanoate (2.30 g, 8.04 mmol, 1.00 equiv) and Boc₂O (2.64 g, 12.07 mmol, 1.50 equiv) in CH₂Cl₂ (30 mL) was added DIEA (3.12 g, 24.14 mmol, 3.00 equiv) dropwise at room temperature under nitrogen atmosphere. The resulting mixture was stirred for 4 h at room temperature under nitrogen atmosphere. The reaction was monitored by LCMS. The resulting mixture was diluted with water (200 mL). The resulting mixture was extracted with CH₂Cl₂ (3×200 mL). The combined organic layers were washed with brine (1×200 mL), dried over anhydrous Na₂SO₄, filtered and concentrated. The crude product was purified by silica gel column chromatography, eluted with PE/EtOAc (0%-10% gradient in 30 min) to afford ethyl (3S)-3-[(tert-butoxycarbonyl)amino]-3-(5-chloro-3-cyclopropyl-2-fluorophenyl)propanoate (2.6 g, 79.5%) as a white solid. LC-MS: (ES−H, m/z): [M−H]⁻=384.0. ¹H NMR (300 MHz, DMSO-d₆) δ 7.58 (d, J=8.8 Hz, 1H), 7.23 (dd, J=6.0, 2.6 Hz, 1H), 6.92 (dd, J=6.2, 2.6 Hz, 1H), 5.26-5.18 (m, 1H), 4.06-4.02 (m, 2H), 2.69-2.65 (m, 2H), 2.05-2.02 (m, 1H), 1.36 (s, 9H), 1.13 (t, J=7.1 Hz, 3H), 1.01-0.97 (m, 2H), 0.77 (t, J=5.1 Hz, 2H).

### Step 7: Preparation of ethyl (3S)-3-[(tert-butoxycarbonyl)amino]-3-[3-cyclopropyl-2-fluoro-5-(4,4,5,5-tetramethyl-1,3,2-dioxaborolan-2-yl)phenyl]propanoate

To a stirred solution of ethyl (3S)-3-[(tert-butoxycarbonyl)amino]-3-(5-chloro-3-cyclopropyl-2-fluorophenyl)propanoate (2.60 g, 6.73 mmol, 1.00 equiv) and 4,4,5,5-tetramethyl-2-(4,4,5,5-tetramethyl-1,3,2-dioxaborolan-2-yl)-1,3,2-dioxaborolane (2.05 g, 8.08 mmol, 1.20 equiv) in dioxane (40 mL) was added XPhos Pd G3 (0.57 g, 0.67 mmol, 0.10 equiv), XPhos (0.64 g, 1.34 mmol, 0.20 equiv) and KOAc (1.98 g, 20.21 mmol, 3.00 equiv) at room temperature under nitrogen atmosphere. The resulting mixture was stirred for 2 h at 100° C. under nitrogen atmosphere. The reaction was monitored by LCMS. The mixture was allowed to cool down to room temperature. The resulting mixture was filtered, the filter cake was washed with EtOAc (3×200 mL). The filtrate was concentrated under reduced pressure. The residue was purified by silica gel column chromatography, eluted with PE/EtOAc (0%-30% gradient in 30 min) to afford ethyl (3S)-3-[(tert-butoxycarbonyl)amino]-3-[3-cyclopropyl-2-fluoro-5-(4,4,5,5-tetramethyl-1,3,2-dioxaborolan-2-yl)phenyl]propanoate (3 g, 74.6%) as a red oil. LC-MS: (ES+H, m/z): [M+H−tBu]⁺=422.2. ¹H NMR (300 MHz, DMSO-d₆) δ 7.67-7.51 (m, 2H), 7.15-7.04 (m, 1H), 5.27-5.19 (m, 1H), 4.07-3.99 (m, 2H), 2.75-2.58 (m, 2H), 2.08-2.00 (m, 1H), 1.28 (d, J=2.0 Hz, 12H), 1.12 (d, J=7.1 Hz, 3H), 1.07 (s, 9H), 0.99-096 (m, 2H), 0.71-0.63 (m, 2H).

### Step 8: Preparation of ethyl (3S)-3-[(tert-butoxycarbonyl)amino]-3-{5-cyclopropyl-4-fluoro-2′-hydroxy-6′-methyl-[1,1′-biphenyl]-3-yl}propanoate

To a stirred solution of ethyl (3S)-3-[(tert-butoxycarbonyl)amino]-3-[3-cyclopropyl-2-fluoro-5-(4,4,5,5-tetramethyl-1,3,2-dioxaborolan-2-yl)phenyl]propanoate (1.40 g, 2.93 mmol, 1.00 equiv) and 2-bromo-3-methylphenol (0.66 g, 3.52 mmol, 1.20 equiv) in dioxane (15 mL) and H₂O (1.5 mL) were added Pd(dppf)Cl₂·CH₂Cl₂ (0.21 g, 0.29 mmol, 0.10 equiv) and K₂CO₃ (1.22 g, 8.78 mmol, 3.00 equiv) in portions at room temperature. The resulting mixture was stirred overnight at 100° C. under nitrogen atmosphere. The reaction was monitored by LCMS. The mixture was allowed to cool down to room temperature. The resulting mixture was filtered, the filter cake was washed with EtOAc (3×100 mL). The filtrate was concentrated under reduced pressure. The residue was purified by silica gel column chromatography, eluted with PE/EtOAc (0%-30% gradient in 30 min) to afford ethyl (3S)-3-[(tert-butoxycarbonyl)amino]-3-{5-cyclopropyl-4-fluoro-2′-hydroxy-6′-methyl-[1,1′-biphenyl]-3-yl}propanoate (760 mg, 53.8%) as a red oil. LC-MS: (ES+H, m/z): [M+H]⁺=458.2. ¹H NMR (300 MHz, DMSO-d₆) δ 9.10 (s, 1H), 7.53 (d, J=8.9 Hz, 1H), 7.06-6.95 (m, 2H), 6.72 (t, J=7.8 Hz, 2H), 6.62 (dd, J=7.1, 2.1 Hz, 1H), 5.30 (d, J=8.2 Hz, 1H), 4.12-4.00 (m, 2H), 2.66 (d, J=7.5 Hz, 2H), 2.11-2.04 (m, 1H), 1.93 (s, 3H), 1.34 (s, 9H), 1.14 (t, J=7.1 Hz, 3H), 1.00-0.92 (m, 2H), 0.69 (t, J=4.1 Hz, 2H).

### Step 9: Preparation of ethyl (3S)-3-amino-3-{5-cyclopropyl-4-fluoro-2′-hydroxy-6′-methyl-[1,1′-biphenyl]-3-yl}propanoate

To a stirred solution of ethyl (3S)-3-[(tert-butoxycarbonyl)amino]-3-{5-cyclopropyl-4-fluoro-2′-hydroxy-6′-methyl-[1,1′-biphenyl]-3-yl}propanoate (660 mg, 1.44 mmol, 1.00 equiv) in CH₂Cl₂ (5 mL, 103.78 mmol, 71.97 equiv) was added HCl (gas) in 1,4-dioxane (40 mL, 4 M) dropwise at room temperature. The resulting mixture was stirred for 3 h at room temperature. The reaction was monitored by LCMS. The resulting mixture was concentrated under reduced pressure to provide ethyl (3S)-3-amino-3-{5-cyclopropyl-4-fluoro-2′-hydroxy-6′-methyl-[1,1′-biphenyl]-3-yl}propanoate (540 mg, crude) as a yellow solid. LC-MS: (ES+H, m/z): [M+H]⁺=358.10.

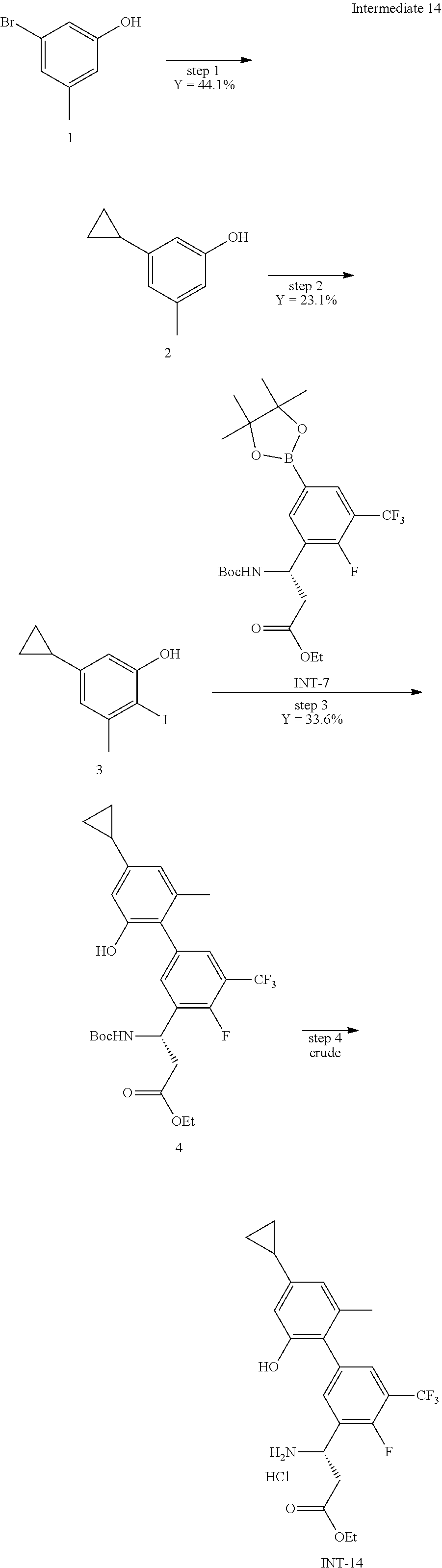

### Step 1: Preparation of 3-cyclopropyl-5-methylphenol

To a stirred mixture of 3-bromo-5-methylphenol (10.00 g, 53.47 mmol, 1.00 equiv) and cyclopropylboronic acid (6.89 g, 80.20 mmol, 1.50 equiv) in toluene (400 mL) and H₂O (40 mL) were added Pd(OAc)₂ (1.20 g, 5.35 mmol, 0.10 equiv), PCy₃ (3.00 g, 10.69 mmol, 0.20 equiv) and K₃PO₄ (34.05 g, 160.40 mmol, 3.00 equiv) at room temperature. The resulting mixture was stirred for 2 h at 100° C. under nitrogen atmosphere. The reaction was monitored by LCMS. Desired product could be detected by LCMS. The mixture was allowed to cool down to room temperature and concentrated. The resulting mixture was diluted with water (200 mL) and extracted with EtOAc (3×200 mL). The combined organic layers were washed with brine (3×200 mL), dried over anhydrous Na₂SO₄. After filtration, the filtrate was concentrated under reduced pressure. The residue was purified by silica gel column chromatography, eluted with PE/EtOAc (0 to 20% gradient in 30 min) to afford 3-cyclopropyl-5-methylphenol (3.5 g, 44.1%) as a yellow liquid. LC-MS: (ES−H, m/z): [M−H]⁻=147.0. ¹H NMR (300 MHz, CDCl₃) δ 6.52 (tt, J=1.4, 0.7 Hz, 1H), 6.46 (tt, J=1.5, 0.7 Hz, 1H), 6.36 (t, J=2.0 Hz, 1H), 4.64 (s, 1H), 2.29 (d, J=0.8 Hz, 3H), 1.83 (tt, J=8.4, 5.1 Hz, 1H), 1.00-0.89 (m, 2H), 0.74-0.63 (m, 2H).

### Step 2: Preparation of 5-cyclopropyl-2-iodo-3-methylphenol

To a stirred solution of 3-cyclopropyl-5-methylphenol (3.50 g, 23.62 mmol, 1.00 equiv) in toluene (200 mL) was added NaH (1.89 g, 47.23 mmol, 2.00 equiv, 60%) in portions at 0° C. under nitrogen atmosphere. The resulting mixture was stirred for 30 min at 0° C. under nitrogen atmosphere. To the above mixture was added Iodine (5.99 g, 23.62 mmol, 1.00 equiv) in portions at 0° C. The resulting mixture was stirred for an additional 1 h at 0° C. The reaction was monitored by LCMS. Desired product could be detected by LCMS. The mixture was allowed to cool down to room temperature. The resulting mixture was diluted with water (100 mL). The mixture was acidified to pH 5 with HCl (aq. 1 N). The resulting mixture was concentrated under reduced pressure. The resulting mixture was extracted with EtOAc (3×200 mL). The combined organic layers were washed with brine (3×200 mL), dried over anhydrous Na₂SO₄. After filtration, the filtrate was concentrated under reduced pressure. The residue was purified by silica gel column chromatography, eluted with PE/CH₂Cl₂ (0 to 20% gradient in 30 min) to afford 5-cyclopropyl-2-iodo-3-methylphenol (1.50 g, 23.1%) as a white liquid. LCMS: (ES−H, m/z): [M−H]⁻=272.9. ¹H NMR (300 MHz, CDCl₃) δ 6.58 (d, J=2.1 Hz, 1H), 6.52 (d, J=2.1 Hz, 1H), 5.34 (s, 1H), 2.39 (t, J=0.7 Hz, 3H), 1.79 (tt, J=8.4, 5.0 Hz, 1H), 0.99-0.91 (m, 2H), 0.71-0.61 (m, 2H).

### Step 3: Preparation of ethyl (S)-3-((tert-butoxycarbonyl)amino)-3-(4′-cyclopropyl-4-fluoro-2′-hydroxy-6′-methyl-5-(trifluoromethyl)-[1,1′-biphenyl]-3-yl)propanoate

To a stirred mixture of ethyl (S)-3-((tert-butoxycarbonyl)amino)-3-(2-fluoro-5-(4,4,5,5-tetramethyl-1,3,2-dioxaborolan-2-yl)-3-(trifluoromethyl)phenyl)propanoate (1.00 g, 1.98 mmol, 1.00 equiv) and 5-cyclopropyl-2-iodo-3-methylphenol (0.65 g, 2.38 mmol, 1.20 equiv) in 1,4-dioxane (20 mL) and H₂O (1 mL) were added Pd(dppf)Cl₂·CH₂Cl₂ (0.14 g, 0.20 mmol, 0.10 equiv) and K₂CO₃ (0.55 g, 3.96 mmol, 2.00 equiv) at room temperature. The resulting mixture was stirred for 2 h at 100° C. under nitrogen atmosphere. The reaction was monitored by LCMS. Desired product could be detected by LCMS. The mixture was allowed to cool down to room temperature. The resulting mixture was diluted with water (200 mL). The resulting mixture was extracted with EtOAc (3×200 mL). The combined organic layers were washed with brine (3×200 mL), dried over anhydrous Na₂SO₄, filtered and concentrated. The crude product was purified by silica gel column chromatography, eluted with PE/EtOAc (0 to 20% gradient in 30 min) to afford the crude product (490 mg) which was further purified by Preparative SFC (Column: NB_CHIRALPAK AD-H 3*25 cm, 5 μm; Mobile Phase A: CO₂, Mobile Phase B: EtOH; Flow rate: 100 mL/min; Gradient: isocratic 25% B; Column Temperature (° C.): 35; Back Pressure (bar): 100; wavelength: 220/202 nm; RT1 (min): 3.03; RT2 (min): 7.18; Sample Solvent: EtOH; Injection Volume: 4 mL) to afford ethyl (S)-3-((tert-butoxycarbonyl)amino)-3-(4′-cyclopropyl-4-fluoro-2′-hydroxy-6′-methyl-5-(trifluoromethyl)-[1,1′-biphenyl]-3-yl)propanoate (350 mg, 33.6%) as a white solid. LC-MS: (ES−H, m/z): [M−H]⁻=524.2. ¹H NMR (300 MHz, CD₃OD) δ 7.47 (dd, J=6.7, 2.2 Hz, 1H), 7.39 (dd, J=6.7, 2.2 Hz, 1H), 6.52 (d, J=1.7 Hz, 1H), 6.44 (d, J=1.7 Hz, 1H), 5.40 (s, 1H), 4.10 (qd, J=7.2, 1.1 Hz, 2H), 2.90-2.70 (m, 2H), 1.98 (s, 3H), 1.81 (tt, J=8.3, 5.0 Hz, 1H), 1.40 (s, 9H), 1.20 (t, J=7.1 Hz, 3H), 1.00-0.87 (m, 2H), 0.77-0.62 (m, 2H).

### Step 4: Preparation of ethyl (S)-3-amino-3-(4′-cyclopropyl-4-fluoro-2′-hydroxy-6′-methyl-5-(trifluoromethyl)-[1,1′-biphenyl]-3-yl)propanoate hydrochloride

To a stirred solution of ethyl (S)-3-((tert-butoxycarbonyl)amino)-3-(4′-cyclopropyl-4-fluoro-2′-hydroxy-6′-methyl-5-(trifluoromethyl)-[1,1′-biphenyl]-3-yl)propanoate (350 mg, 0.67 mmol, 1.00 equiv) in CH₂Cl₂ (5 mL) was added HCl (gas) in 1,4-dioxane (5 mL, 4 M) dropwise at room temperature under nitrogen atmosphere. The resulting mixture was stirred for 1 h at room temperature under nitrogen atmosphere. The reaction was monitored by LCMS. Desired product could be detected by LCMS. The resulting mixture was concentrated under reduced pressure to afford ethyl (S)-3-amino-3-(4′-cyclopropyl-4-fluoro-2′-hydroxy-6′-methyl-5-(trifluoromethyl)-[1,1′-biphenyl]-3-yl)propanoate hydrochloride (400 mg, crude) as a white oil. LC-MS: (ES+H, m/z): [M+H]⁺=426.1

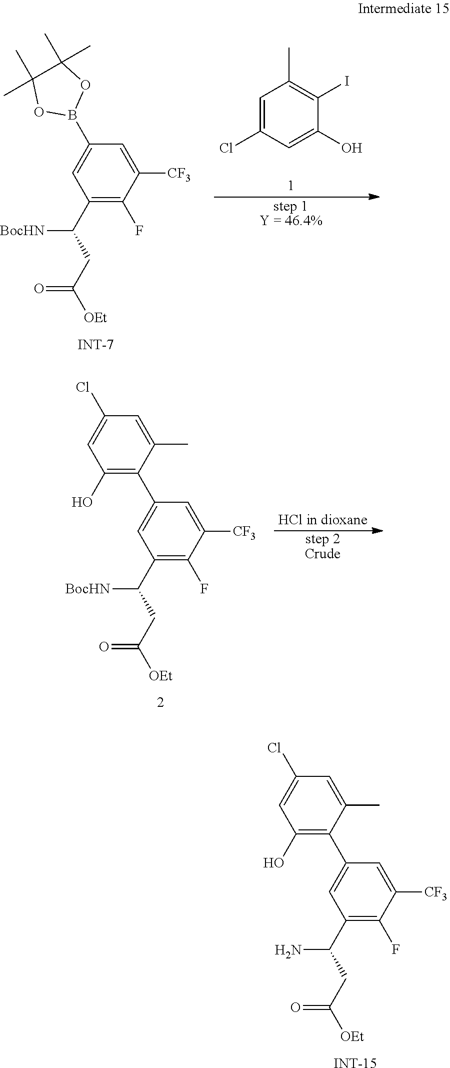

### Step 1: Preparation of ethyl (S)-3-((tert-butoxycarbonyl)amino)-3-(4′-chloro-4-fluoro-2′-hydroxy-6′-methyl-5-(trifluoromethyl)-[1,1′-biphenyl]-3-yl)propanoate

A mixture of ethyl (3S)-3-[(tert-butoxycarbonyl)amino]-3-[2-fluoro-5-(4,4,5,5-tetramethyl-1,3,2-dioxaborolan-2-yl)-3-(trifluoromethyl)phenyl]propanoate (700 mg, 1.39 mmol, 1.00 equiv), 5-chloro-2-iodo-3-methylphenol (446 mg, 1.66 mmol, 1.20 equiv), Pd(PPh₃)₄ (160 mg, 0.13 mmol, 0.10 equiv) and K₂HPO₄ (723 mg, 4.15 mmol, 3.00 equiv) in 1,4-dioxane/water=10/1 (11 mL) was stirred overnight at 90° C. under nitrogen atmosphere. Desired product could be detected by LCMS. The mixture was allowed to cool down to room temperature. The resulting mixture was diluted with water (30 mL). The resulting mixture was diluted with water (30 mL and extracted with EtOAc (3×30 mL). The combined organic layers were concentrated under reduced pressure and the crude product was purified by reversed-phase flash chromatography (C18; mobile phase, MeCN in water (0.1% NH3·H2O), 30% to 60% gradient in 30 min; detector, UV 220 nm) to afford ethyl (S)-3-((tert-butoxycarbonyl)amino)-3-(4′-chloro-4-fluoro-2′-hydroxy-6′-methyl-5-(trifluoromethyl)-[1,1′-biphenyl]-3-yl)propanoate (270 mg, 46.4%) as a white solid. LC-MS: (ES+H, m/z): [M+H−tBu]⁺=464.1.

### Step 2: Preparation of ethyl (S)-3-amino-3-(4′-chloro-4-fluoro-2′-hydroxy-6′-methyl-5-(trifluoromethyl)-[1,1′-biphenyl]-3-yl)propanoate

A solution of ethyl (3S)-3-[(tert-butoxycarbonyl)amino]-3-[4′-chloro-4-fluoro-2′-hydroxy-6′-methyl-5-(trifluoromethyl)-[1,1′-biphenyl]-3-yl]propanoate (300 mg, 0.57 mmol, 1.00 equiv) and HCl (gas) in 1,4-dioxane (5 mL, 4 M) in CH₂Cl₂ (2 mL) was stirred for 1 h at room temperature under nitrogen atmosphere. Desired product could be detected by LCMS. The resulting mixture was concentrated under reduced pressure to provide the HCl salt of ethyl (S)-3-amino-3-(4′-chloro-4-fluoro-2′-hydroxy-6′-methyl-5-(trifluoromethyl)-[1,1′-biphenyl]-3-yl)propanoate (300 mg, crude) as an off-white solid. LC-MS: (ES−H, m/z): [M−H]⁻=418.1.

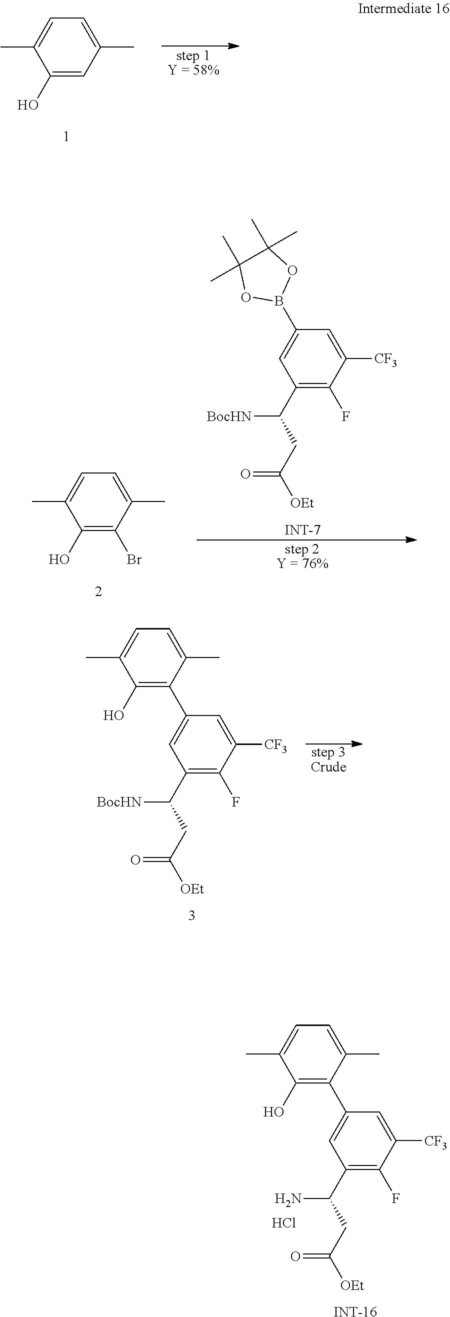

### Step 1: Preparation of 2-bromo-3,6-dimethylphenol

To a solution of 2,5-xylenol (11.00 g, 90.04 mmol, 1.00 equiv) and bis(propan-2-yl)amine (0.91 g, 9.00 mmol, 0.10 equiv) in CH₂Cl₂ (800 mL) were added NBS (16.03 g, 90.04 mmol, 1.00 equiv) in CH₂Cl₂ (800 mL) dropwise over a period of 3 h. The resulting mixture was stirred for an additional 1 h at room temperature. The reaction was monitored by TLC (PE:EtOAc=8:1, Rf=0.5). The mixture was acidified to pH 1 with conc. HCl (1 M), and the resulting phases were separated. The organic phase was washed with H₂O (200 mL), dried over Na₂SO₄. The residue was purified by silica gel column chromatography, eluted with PE/EtOAc (5% to 20% gradient in 20 min) to afford 2-bromo-3,6-dimethylphenol (10.50 g, 58.0%) as colorless liquid. ¹H NMR (400 MHz, CDCl₃): δ 6.94 (d, J=7.6 Hz, 1H), 6.70 (d, J=7.6 Hz, 1H), 5.66 (s, 1H), 2.35 (s, 3H), 2.26 (s, 3H).

### Step 2: Preparation of ethyl (3S)-3-[(tert-butoxycarbonyl)amino]-3-[4-fluoro-2′-hydroxy-3′,6′-dimethyl-5-(trifluoromethyl)-[1,1′-biphenyl]-3-yl]propanoate

To a stirred solution of ethyl (3S)-3-[(tert-butoxycarbonyl)amino]-3-[2-fluoro-5-(4,4,5,5-tetramethyl-1,3,2-dioxaborolan-2-yl)-3-(trifluoromethyl)phenyl]propanoate (610 mg, 1.20 mmol, 1.00 equiv) and 2-bromo-3,6-dimethylphenol (242 mg, 1.20 mmol, 1.00 equiv) in 1,4-dioxane (10 mL) and H₂O (0.5 mL) were added Pd(dppf)Cl₂·CH₂Cl₂ (98 mg, 0.12 mmol, 0.10 equiv) and K₂CO₃ (500 mg, 3.62 mmol, 3.00 equiv) at room temperature under nitrogen atmosphere. The resulting mixture was stirred for an additional 2 h at 100° C. The reaction was monitored by LCMS. The mixture was allowed to cool down to room temperature. The resulting mixture was diluted with water (30 mL). The resulting mixture was extracted with EtOAc (3×60 mL). The combined organic layers were washed with brine (1×10 mL), dried over anhydrous Na₂SO₄. After filtration, the filtrate was concentrated under reduced pressure. The residue was purified by silica gel column chromatography, eluted with PE/EtOAc (10% to 50% gradient in 10 min) to afford ethyl (3S)-3-[(tert-butoxycarbonyl)amino]-3-[4-fluoro-2′-hydroxy-3′,6′-dimethyl-5-(trifluoromethyl)-[1,1′-biphenyl]-3-yl]propanoate (460 mg, 76.2%) as a yellow oil. LC-MS: (ES+H, m/z): [M+Na]⁺=522.2. ¹H NMR (400 MHz, CDCl₃): δ 7.52 (dd, J=6.8, 2.1 Hz, 1H), 7.45 (dd, J=6.7, 2.1 Hz, 1H), 7.06 (d, J=7.6 Hz, 1H), 6.77 (d, J=7.6 Hz, 1H), 5.79 (s, 1H), 5.35 (d, J=39.8 Hz, 1H), 4.76-4.52 (m, 1H), 4.10-4.00 (m, 2H), 2.91 (d, J=5.6 Hz, 2H), 2.25 (s, 3H), 1.99 (s, 3H), 1.41 (s, 9H), 1.19 (t, J=7.1 Hz, 3H). ¹⁹F NMR (376 MHz, CDCl₃): δ −61.09, −61.12, −121.42.

### Step 3: Preparation of ethyl (3S)-3-amino-3-[4-fluoro-2′-hydroxy-3′,6′-dimethyl-5-(trifluoromethyl)-[1,1′-biphenyl]-3-yl]propanoate hydrochloride

To a stirred solution of ethyl (3S)-3-[(tert-butoxycarbonyl)amino]-3-[4-fluoro-2′-hydroxy-3′,6′-dimethyl-5-(trifluoromethyl)-[1,1′-biphenyl]-3-yl]propanoate (430 mg, 0.86 mmol, 1.00 equiv) in CH₂Cl₂ (7 mL) was added HCl (gas) in 1,4-dioxane (5 mL, 4 M) dropwise at 0° C. under nitrogen atmosphere. The resulting mixture was stirred for additional 1.5 h at room temperature. The reaction was monitored by LCMS. Desired product could be detected by LCMS. The resulting mixture was concentrated under reduced pressure. This resulted in ethyl (3S)-3-amino-3-[4-fluoro-2′-hydroxy-3′,6′-dimethyl-5-(trifluoromethyl)-[1,1′-biphenyl]-3-yl]propanoate hydrochloride (430 mg, crude) as yellow solid. LC-MS: (ES+H, m/z): [M+H]⁺=400.3

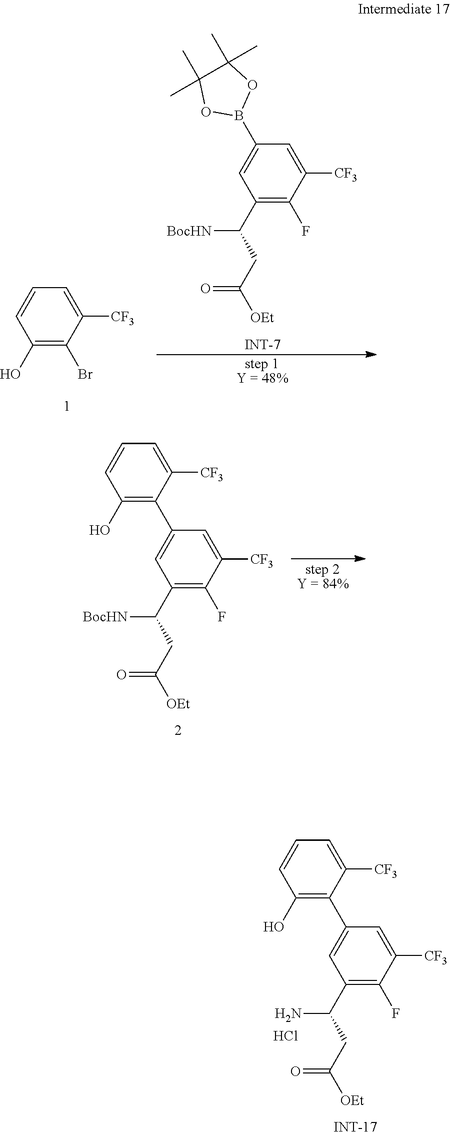

### Step 1: Preparation of ethyl (3S)-3-[(tert-butoxycarbonyl)amino]-3-[4-fluoro-2′-hydroxy-5,6′-bis(trifluoromethyl)-[1,1′-biphenyl]-3-yl]propanoate

To a stirred solution of ethyl (3S)-3-[(tert-butoxycarbonyl)amino]-3-[2-fluoro-5-(4,4,5,5-tetramethyl-1,3,2-dioxaborolan-2-yl)-3-(trifluoromethyl)phenyl]propanoate (1.00 g, 1.97 mmol, 1.00 equiv) and 2-bromo-3-(trifluoromethyl)phenol (0.52 g, 2.17 mmol, 1.10 equiv) in dioxane (10 mL) were added Pd(dppf)Cl₂·CH₂Cl₂ (109 mg, 0.19 mmol, 0.10 equiv) and K₂HPO₄ (1.03 g, 5.93 mmol, 3.00 equiv) at room temperature under nitrogen atmosphere. The resulting mixture was stirred overnight at 90° C. under nitrogen atmosphere. The reaction was monitored by LCMS. The mixture was allowed to cool down to room temperature. The resulting mixture was diluted with EtOAc (200 mL). The residue was washed with water (2×20 mL), dried over anhydrous Na₂SO₄, filtered and concentrated. The crude product was purified by silica gel column chromatography, eluted with PE/EtOAc (0% to 30% gradient in 30 min) to afford ethyl (3S)-3-[(tert-butoxycarbonyl)amino]-3-[4-fluoro-2′-hydroxy-5,6′-bis(trifluoromethyl)-[1,1′-biphenyl]-3-yl]propanoate (510 mg, 47.7%) as a yellow oil. LC-MS: (ES+H, m/z): [M+H−tBu]⁺=484.0.

### Step 2: Preparation of ethyl (3S)-3-amino-3-[4-fluoro-2′-hydroxy-5,6′-bis(trifluoromethyl)-[1,1′-biphenyl]-3-yl]propanoate

To a stirred solution of ethyl (3S)-3-[(tert-butoxycarbonyl)amino]-3-[4-fluoro-2′-hydroxy-5,6′-bis(trifluoromethyl)-[1,1′-biphenyl]-3-yl]propanoate (510 mg, 0.94 mmol, 1.00 equiv) in CH₂C₂ (6 mL) was added HCl (gas) in 1,4-dioxane (3 mL, 4 M) dropwise at 0° C. under nitrogen atmosphere. The resulting mixture was stirred for 3 h at room temperature under nitrogen atmosphere. The reaction was monitored by LCMS. The resulting mixture was concentrated under reduced pressure. This resulted in ethyl (3S)-3-amino-3-[4-fluoro-2′-hydroxy-5,6′-bis(trifluoromethyl)-[1,1′-biphenyl]-3-yl]propanoate (350 mg, 84.3%) as a yellow oil. LC-MS: (ES+H, m/z): [M+H]⁺=440.0

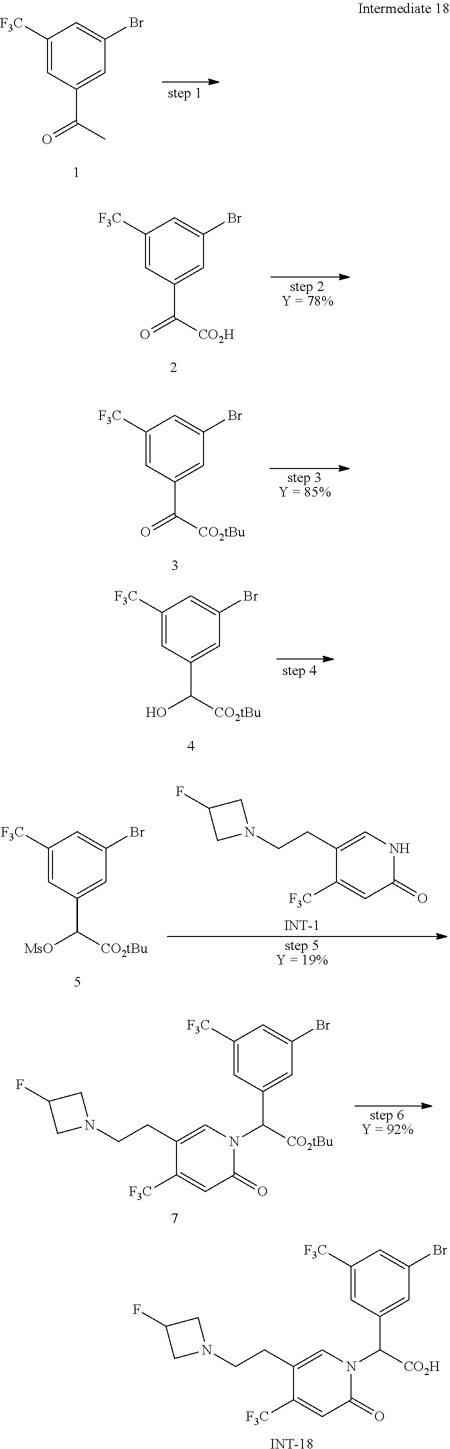

### Step 1: Preparation of [3-bromo-5-(trifluoromethyl)phenyl](oxo)acetic acid

To a stirred mixture of 1-[3-bromo-5-(trifluoromethyl)phenyl]ethanone (4.00 g, 14.97 mmol, 1.00 equiv) in pyridine (40 mL) was added SeO₂ (2.49 g, 22.46 mmol, 1.50 equiv) at room temperature under nitrogen atmosphere. The resulting mixture was stirred for 2 h at 100° C. under nitrogen atmosphere. The reaction was monitored by LCMS. The mixture was acidified to pH 4 with conc. HCl. The resulting mixture was filtered, the filter cake was washed with EtOAc (3×150 mL). The resulting mixture was extracted with EtOAc (2×500 mL). The combined organic layers were washed with brine (1×500 mL), dried over anhydrous Na₂SO₄, filtered and concentrated to afford [3-bromo-5-(trifluoromethyl)phenyl](oxo)acetic acid (4.7 g, crude) as a red oil. LC-MS: (ES−H, m/z): [M−H]⁻=294.8. ¹H NMR (400 MHz, DMSO-d₆) δ 8.39 (q, J=1.9 Hz, 2H), 8.24 (s, 1H).

### Step 2: Preparation of tert-butyl 2-[3-bromo-5-(trifluoromethyl)phenyl]-2-oxoacetate

To a stirred mixture of [3-bromo-5-(trifluoromethyl)phenyl](oxo)acetic acid (4.00 g, 13.46 mmol, 1.00 equiv) in CH₂Cl₂ (200 mL) was added (E)-N,N′-bis(propan-2-yl)(tert-butoxy)methanimidamide (5.40 g, 26.93 mmol, 2.00 equiv) at room temperature under nitrogen atmosphere. The resulting mixture was stirred overnight at room temperature under nitrogen atmosphere. The reaction was monitored by LCMS. The resulting mixture was filtered, the filter cake was washed with EtOAc (3×150 mL). The filtrate was concentrated under reduced pressure. The residue was purified by silica gel column chromatography, eluted with PE/EtOAc (0% to 10% gradient in 30 min) to afford tert-butyl 2-[3-bromo-5-(trifluoromethyl)phenyl]-2-oxoacetate (3.7 g, 77.8%) as a yellow oil. ¹H NMR (400 MHz, DMSO-d₆) δ 8.40-8.32 (m, 2H), 8.20 (td, J=1.6, 0.8 Hz, 1H), 1.57 (s, 9H).

### Step 3: Preparation of tert-butyl 2-[3-bromo-5-(trifluoromethyl)phenyl]-2-hydroxyacetate

To a stirred mixture of tert-butyl 2-[3-bromo-5-(trifluoromethyl)phenyl]-2-oxoacetate (3.5 g, 9.91 mmol, 1.00 equiv) and NaBH₃CN (685 mg, 10.90 mmol, 1.10 equiv) in CH₃CH₂OH (15 mL) was added CH₃COOH (1.5 mL) at room temperature under nitrogen atmosphere. The resulting mixture was stirred for 1 h at room temperature under nitrogen atmosphere. The reaction was monitored by LCMS. The reaction was quenched with water (300 ml) at room temperature. The resulting mixture was extracted with EtOAc (3×500 mL). The combined organic layers were washed with brine (1×500 mL), dried over anhydrous Na₂SO₄, filtered and concentrated. The crude product was purified by silica gel column chromatography, eluted with PE/EtOAc (0% to 20% gradient in 30 min) to afford tert-butyl 2-[3-bromo-5-(trifluoromethyl)phenyl]-2-hydroxyacetate (3 g, 85.2%) as a white solid. LC-MS: (ES−H, m/z): [M−H]⁻=352.9. ¹H NMR (300 MHz, DMSO-d₆) δ 7.92 (d, J=4.2 Hz, 2H), 7.80-7.60 (m, 1H), 6.34 (d, J=5.8 Hz, 1H), 5.21 (d, J=5.8 Hz, 1H), 1.35 (s, 9H).

### Step 4: Preparation of tert-butyl 2-[3-bromo-5-(trifluoromethyl)phenyl]-2-(methanesulfonyloxy)acetate

To a stirred mixture of tert-butyl 2-[3-bromo-5-(trifluoromethyl)phenyl]-2-hydroxyacetate (1.00 g, 2.81 mmol, 1.00 equiv) and NEt₃ (427 mg, 4.22 mmol, 1.50 equiv) in CH₂Cl₂ (10 mL) was added MsCl (387 mg, 3.37 mmol, 1.20 equiv) dropwise at room temperature under nitrogen atmosphere. The resulting mixture was stirred for 1 h at room temperature under nitrogen atmosphere. The reaction was monitored by LCMS. The reaction was quenched with water (100 mL) at room temperature. The resulting mixture was diluted with EtOAc (200 mL). The combined organic layers were washed with brine (3×100 mL), dried over anhydrous Na₂SO₄, filtered, and concentrated to afford tert-butyl 2-[3-bromo-5-(trifluoromethyl)phenyl]-2-(methanesulfonyloxy)acetate (1.5 g, crude) as a white solid. ¹H NMR (300 MHz, DMSO-d₆) δ 8.13 (s, 1H), 8.00 (s, 1H), 7.82 (s, 1H), 6.27 (s, 1H), 3.36 (s, 3H), 1.39 (s, 9H).

### Step 5: Preparation of tert-butyl 2-[3-bromo-5-(trifluoromethyl)phenyl]-2-{5-[2-(3-fluoroazetidin-1-yl)ethyl]-2-oxo-4-(trifluoromethyl)pyridin-1-yl}acetate

To a stirred mixture of tert-butyl 2-[3-bromo-5-(trifluoromethyl)phenyl]-2-(methanesulfonyloxy)acetate (1.49 g, 3.44 mmol, 1.30 equiv) and 5-[2-(3-fluoroazetidin-1-yl)ethyl]-4-(trifluoromethyl)-1H-pyridin-2-one (700 mg, 2.64 mmol, 1.00 equiv) in MeCN (20 mL) was added K₂CO₃ (732 mg, 5.29 mmol, 2.00 equiv) at room temperature under nitrogen atmosphere. The resulting mixture was stirred for 2 h at 80° C. under nitrogen atmosphere. The reaction was monitored by LCMS. The resulting mixture was filtered, the filter cake was washed with EtOAc (3×100 mL). The filtrate was concentrated under reduced pressure. The residue was purified by reversed-phase flash chromatography (C18; mobile phase, MeCN in water (0.1% NH₃·H₂O), 10% to 50% gradient in 10 min; detector, UV 220 nm) to afford tert-butyl 2-[3-bromo-5-(trifluoromethyl)phenyl]-2-{5-[2-(3-fluoroazetidin-1-yl)ethyl]-2-oxo-4-(trifluoromethyl)pyridin-1-yl}acetate (300 mg, 18.8%) as a white solid. LC-MS: (ES+H, m/z): [M+H]⁺=601.0. ¹H NMR (300 MHz, DMSO-d₆) δ 8.08 (s, 1H), 8.04 (d, J=2.1 Hz, 1H), 7.93 (s, 1H), 7.81 (s, 1H), 6.88 (s, 1H), 6.33 (s, 1H), 5.06 (dp, J=57.8, 5.1 Hz, 1H), 3.49 (dq, J=14.6, 7.2 Hz, 2H), 3.12-2.93 (m, 2H), 2.59-2.53 (m, 2H), 2.44 (t, J=6.8 Hz, 2H), 1.40 (s, 9H).

### Step 6: Preparation of [3-bromo-5-(trifluoromethyl)phenyl]({5-[2-(3-fluoroazetidin-1-yl)ethyl]-2-oxo-4-(trifluoromethyl)pyridin-1-yl})acetic acid

To a stirred mixture of tert-butyl 2-[3-bromo-5-(trifluoromethyl)phenyl]-2-{5-[2-(3-fluoroazetidin-1-yl)ethyl]-2-oxo-4-(trifluoromethyl)pyridin-1-yl}acetate (300 mg, 0.49 mmol, 1.00 equiv) in CH₂Cl₂ (6 mL) was added CF₃CO₂H (6 mL) at room temperature under nitrogen atmosphere. The resulting mixture was stirred for 3 h at room temperature under nitrogen atmosphere. The reaction was monitored by LCMS. The resulting mixture was concentrated under reduced pressure. The residue was purified by reversed-phase flash chromatography (C18; mobile phase, MeCN in water (0.1% NH₃·H₂O), 0 to 100% gradient in 20 min; detector, UV 220 nm) to afford [3-bromo-5-(trifluoromethyl)phenyl]({5-[2-(3-fluoroazetidin-1-yl)ethyl]-2-oxo-4-(trifluoromethyl)pyridin-1-yl})acetic acid (250 mg, 91.9%) as a white solid. LC-MS: (ES+H, m/z): [M+H]⁺=545.0.

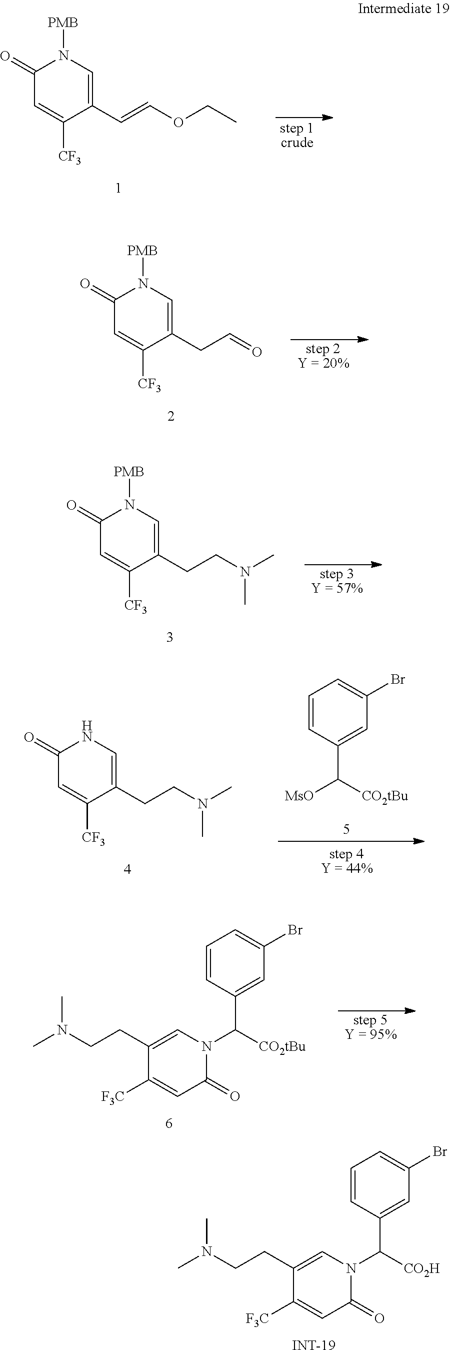

### Step 1: Preparation of 2-{1-[(4-methoxyphenyl)methyl]-6-oxo-4-(trifluoromethyl)pyridin-3-yl}acetaldehyde

To a stirred mixture of 5-[(E)-2-ethoxyethenyl]-1-[(4-methoxyphenyl)methyl]-4-(trifluoromethyl)pyridin-2-one (10.00 g, 28.30 mmol, 1.00 equiv) in CH₂Cl₂ (30 mL) was added CF₃COOH (30 mL) at room temperature under nitrogen atmosphere. The resulting mixture was stirred for 5 h at room temperature under nitrogen atmosphere. The reaction was monitored by LCMS. Desired product could be detected by LCMS. The resulting mixture was concentrated under reduced pressure. The resulting mixture was diluted with CH₂Cl₂ (200 mL). The resulting mixture was washed with 3×200 mL of saturated NaHCO₃ (aq.). The combined organic layers were washed with brine (2×200 mL), dried over anhydrous Na₂SO₄, filtered and concentrated to afford 2-{1-[(4-methoxyphenyl)methyl]-6-oxo-4-(trifluoromethyl)pyridin-3-yl}acetaldehyde (9 g, crude) as red oil. The crude product was used in the next step directly without further purification. LC-MS: (ES+H, m/z): [M+H]⁺=326.1.

### Step 2: Preparation of 5-[2-(dimethylamino)ethyl]-1-[(4-methoxyphenyl)methyl]-4-(trifluoromethyl)pyridin-2-one

To a stirred mixture of 2-{1-[(4-methoxyphenyl)methyl]-6-oxo-4-(trifluoromethyl)pyridin-3-yl}acetaldehyde (9.00 g, 27.66 mmol, 1.00 equiv) and dimethylamine (28 mL, 55.33 mmol, 2.00 equiv, 2 M in THF) in EtOH (30 mL) were added CH₃COOH (166 mg, 2.76 mmol, 0.10 equiv) at room temperature under nitrogen atmosphere. The resulting mixture was stirred for 1 h at room temperature under nitrogen atmosphere. To the above mixture was added NaBH₃CN (3.48 g, 55.33 mmol, 2.00 equiv) in portions over 10 min at room temperature. The resulting mixture was stirred for an additional 4 h at room temperature. The reaction was monitored by LCMS. Desired product could be detected by LCMS. The reaction was quenched by the addition of water (200 mL) at 0° C. The resulting mixture was extracted with EtOAc (3×300 mL). The combined organic layers were washed with brine (2×200 mL), dried over anhydrous Na₂SO₄, filtered and concentrated. The crude product was purified by silica gel column chromatography, eluted with CH₂Cl₂/MeOH (0˜10%, 20 min) to afford 5-[2-(dimethylamino)ethyl]-1-[(4-methoxyphenyl)methyl]-4-(trifluoromethyl)pyridin-2-one (2 g, 20.4%) as yellow oil. LC-MS: (ES+H, m/z): [M+H]⁺=355.1. ¹H NMR (400 MHz, DMSO-d₆) δ 7.91 (s, 1H), 7.25-7.18 (m, 2H), 6.85-6.78 (m, 2H), 6.69 (s, 1H), 4.94 (s, 2H), 3.63 (s, 3H), 2.60-2.55 (m, 2H), 2.42-2.40 (m, 6H), 2.35 (s, 2H). ¹⁹F NMR (377 MHz, DMSO-d₆) δ −62.51.

### Step 3: Preparation of 5-[2-(dimethylamino)ethyl]-4-(trifluoromethyl)-1H-pyridin-2-one

To a stirred mixture of 5-[2-(dimethylamino)ethyl]-1-[(4-methoxyphenyl)methyl]-4-(trifluoromethyl)pyridin-2-one (2.00 g, 5.64 mmol, 1.00 equiv) in CF₃COOH (10 mL) was stirred overnight at 80° C. under nitrogen atmosphere. The mixture was allowed to cool down to room temperature. The reaction was monitored by LCMS. Desired product could be detected by LCMS. The resulting mixture was concentrated under reduced pressure. The residue was purified by reversed-phase flash chromatography (C18; mobile phase, MeCN in water (10 mmol/L NH₄HCO₃), 10% to 50% gradient in 25 min; detector, UV 254 nm/220 nm) to afford 5-[2-(dimethylamino)ethyl]-4-(trifluoromethyl)-1H-pyridin-2-one (750 mg, 56.7%) as a colorless oil. LC-MS: (ES+H, m/z): [M+H]⁺=235.0. ¹H NMR (400 MHz, DMSO-d₆) δ 12.10 (s, 1H), 7.54 (s, 1H), 6.68 (s, 1H), 2.56 (t, J=7.5 Hz, 2H), 2.37 (t, J=8.3, 6.7 Hz, 2H), 2.16 (s, 6H). ¹⁹F NMR (377 MHz, DMSO-d₆) δ −62.65.

### Step 4: Preparation of tert-butyl 2-(3-bromophenyl)-2-{5-[2-(dimethylamino)ethyl]-2-oxo-4-(trifluoromethyl)pyridin-1-yl}acetate

To a stirred mixture of 5-[2-(dimethylamino)ethyl]-4-(trifluoromethyl)-1H-pyridin-2-one (750 mg, 3.20 mmol, 1.00 equiv) and tert-butyl 2-(3-bromophenyl)-2-(methanesulfonyloxy)acetate (1.52 g, 4.16 mmol, 1.30 equiv) in MeCN (10 mL) were added K₂CO₃ (885 mg, 6.40 mmol, 2.00 equiv) at room temperature under nitrogen atmosphere. The resulting mixture was stirred for 2 h at 60° C. under nitrogen atmosphere. The mixture was allowed to cool down to room temperature. The reaction was monitored by LCMS. Desired product could be detected by LCMS. The resulting mixture was extracted with EtOAc (2×100 mL). The combined organic layers were washed with brine (2×100 mL), dried over anhydrous Na₂SO₄. After filtration, the filtrate was concentrated under reduced pressure. The residue was purified by reversed combi-flash chromatography (C18; mobile phase, MeCN in water (10 mmol/L NH₄HCO₃), 10% to 50% gradient in 10 min; detector, UV 254 nm/220 nm) to afford tert-butyl 2-(3-bromophenyl)-2-{5-[2-(dimethylamino)ethyl]-2-oxo-4-(trifluoromethyl)pyridin-1-yl}acetate (710 mg, 44.0%) as an off-white solid. LC-MS: (ES+H, m/z): [M+H]⁺=503.0. ¹H NMR (400 MHz, DMSO-d₆) δ 7.69-7.65 (m, 2H), 7.57 (s, 1H), 7.47-7.42 (m, 2H), 6.85 (s, 1H), 6.27 (s, 1H), 2.52-2.51 (m, 2H), 2.34-2.23 (m, 2H), 2.02 (s, 6H), 1.41 (s, 9H). ¹⁹F NMR (377 MHz, DMSO-d₆) δ −62.70.

### Step 5: Preparation of (3-bromophenyl)({5-[2-(dimethylamino)ethyl]-2-oxo-4-(trifluoromethyl)pyridin-1-yl})acetic acid

To a stirred mixture of tert-butyl 2-(3-bromophenyl)-2-{5-[2-(dimethylamino)ethyl]-2-oxo-4-(trifluoromethyl)pyridin-1-yl}acetate (710 mg, 1.41 mmol, 1.00 equiv) in CH₂Cl₂ (5 mL) was added CF₃COOH (5 mL) at room temperature under nitrogen atmosphere. The resulting mixture was stirred for 2 h at room temperature under nitrogen atmosphere. The reaction was monitored by LCMS. Desired product could be detected by LCMS. The resulting mixture was concentrated under reduced pressure. The residue was purified by reversed combi-flash chromatography (C18; mobile phase, MeCN in water (0.1% HCOOH), 10% to 50% gradient in 10 min; detector, UV 254 nm/220 nm) to afford (3-bromophenyl)({5-[2-(dimethylamino)ethyl]-2-oxo-4-(trifluoromethyl)pyridin-1-yl})acetic acid (600 mg, 95.1%) as an off-white solid. LC-MS: (ES+H, m/z): [M+H]⁺=447.0.

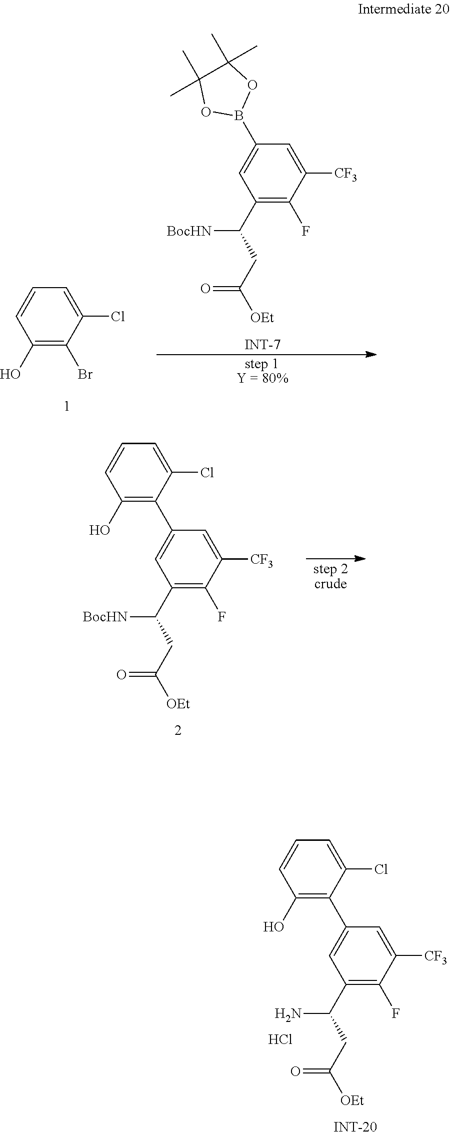

### Step 1: Preparation of ethyl (3S)-3-[(tert-butoxycarbonyl)amino]-3-[2′-chloro-4-fluoro-6′-hydroxy-5-(trifluoromethyl)-[1,1′-biphenyl]-3-yl]propanoate

A mixture of ethyl (3S)-3-[(tert-butoxycarbonyl)amino]-3-[2-fluoro-5-(4,4,5,5-tetramethyl-1,3,2-dioxaborolan-2-yl)-3-(trifluoromethyl)phenyl]propanoate (500 mg, 0.98 mmol, 1.00 equiv), 2-bromo-3-chlorophenol (246 mg, 1.187 mmol, 1.20 equiv), Pd(dppf)Cl₂·CH₂Cl₂ (80 mg, 0.10 mmol, 0.10 equiv) and K₂HPO₄ (517.03 mg, 2.96 mmol, 3.00 equiv) in 1,4-dioxane (10 mL) and H₂O (1 mL) was stirred overnight at 100° C. under nitrogen atmosphere. The mixture was allowed to cool down to room temperature. The reaction was monitored by LCMS. The resulting mixture was concentrated under reduced pressure. The resulting mixture was diluted with water (100 mL). The resulting mixture was extracted with EtOAc (3×100 mL). The combined organic layers were washed with brine (3×100 mL), dried over anhydrous Na₂SO₄. After filtration, the filtrate was concentrated under reduced pressure. The residue was purified by silica gel column chromatography, eluted with PE/EtOAc (0 to 30% gradient in 30 min) to afford ethyl (3S)-3-[(tert-butoxycarbonyl)amino]-3-[2′-chloro-4-fluoro-6′-hydroxy-5-(trifluoromethyl)-[1,1′-biphenyl]-3-yl]propanoate (400 mg, 79.9%) as a yellow oil. LC-MS: (ES+H, m/z): [M+Na]⁺=528.1.

### Step 2: Preparation of ethyl (S)-3-amino-3-(2′-chloro-4-fluoro-6′-hydroxy-5-(trifluoromethyl)-[1,1′-biphenyl]-3-yl)propanoate hydrochloride

A solution of ethyl (3S)-3-[(tert-butoxycarbonyl)amino]-3-[2′-chloro-4-fluoro-6′-hydroxy-5-(trifluoromethyl)-[1,1′-biphenyl]-3-yl]propanoate (350 mg, 0.69 mmol, 1.00 equiv) and HCl (gas) in 1,4-dioxane (2.5 mL, 4 M) in CH₂Cl₂ (2.5 mL) was stirred for 2 h at room temperature under nitrogen atmosphere. The reaction was monitored by LCMS. The resulting mixture was concentrated under reduced pressure. This resulted in ethyl (S)-3-amino-3-(2′-chloro-4-fluoro-6′-hydroxy-5-(trifluoromethyl)-[1,1′-biphenyl]-3-yl)propanoate hydrochloride (350 mg, crude) as a light brown oil. LC-MS: (ES+H, m/z): [M+H]⁺=406.0.

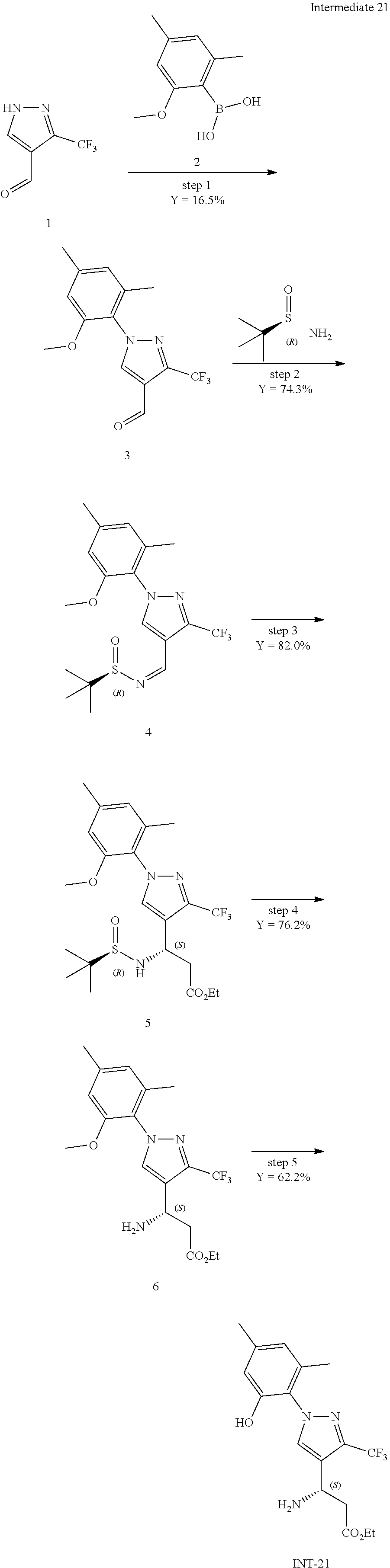

### Step 1: Preparation of 1-(2-methoxy-4,6-dimethylphenyl)-3-(trifluoromethyl)pyrazole-4-carbaldehyde

To a stirred solution of 2-methoxy-4,6-dimethylphenylboronic acid (10.97 g, 60.94 mmol, 2.00 equiv.) and 3-(trifluoromethyl)-1H-pyrazole-4-carbaldehyde (5.00 g, 30.47 mmol, 1.00 equiv.) in DMF (30 mL) were added Cu(OAc)₂ (6.09 g, 33.52 mmol, 1.10 equiv.) and pyridine (289 mg, 3.65 mmol, 2.00 equiv.) dropwise at room temperature under nitrogen atmosphere. The resulting mixture was stirred overnight at room temperature under nitrogen atmosphere and then filtered. The filter cake was washed with EtOAc (2×300 mL), the combined organic layers were concentrated under vacuum, and the crude product was purified by silica gel column chromatography (PE/EtOAc (0% to 15% gradient in 30 min)) to afford 1-(2-methoxy-4,6-dimethylphenyl)-3-(trifluoromethyl)pyrazole-4-carbaldehyde (1.5 g, 16.5%) as a yellow oil. LC-MS: (ES+H, m/z) [M+H]⁺=299.0. ¹H NMR (300 MHz, DMSO-d₆) δ 9.95 (d, J=0.8 Hz, 1H), 8.88 (d, J=1.0 Hz, 1H), 6.96 (d, J=1.7 Hz, 1H), 6.84 (dd, J=1.8, 0.9 Hz, 1H), 3.75 (s, 3H), 2.37 (s, 3H), 1.96 (s, 3H).

### Step 2: Preparation of (R)—N-[(1Z)-[1-(2-methoxy-4,6-dimethylphenyl)-3-(trifluoromethyl)pyrazol-4-yl]methylidene]-2-methylpropane-2-sulfinamide

To a stirred solution of 1-(2-methoxy-4,6-dimethylphenyl)-3-(trifluoromethyl)pyrazole-4-carbaldehyde (1.50 g, 5.02 mmol, 1.00 equiv.) and (R)-2-methylpropane-2-sulfinamide (0.91 g, 7.54 mmol, 1.50 equiv.) in THF (30 mL) was added Ti(Oi-Pr)₄ (2.86 g, 10.05 mmol, 2.00 equiv.) dropwise at room temperature under nitrogen atmosphere. The resulting mixture was stirred for 2 h at 60° C. under nitrogen atmosphere. The mixture was cooled to room temperature, diluted with EtOAc (50 mL) and quenched with water (50 mL). The resulting mixture was filtered, and the filter cake was washed with EtOAc (3×50 mL). The aqueous layer was extracted with EtOAc (2×50 mL) and the combined organic layers were washed with brine (2×20 mL), dried over anhydrous Na₂SO₄, filtered and concentrated. The residue was purified by silica gel column chromatography (PE/EtOAc (0% to 50% gradient in 30 min)) to afford (R)—N-[(1Z)-[1-(2-methoxy-4,6-dimethylphenyl)-3-(trifluoromethyl)pyrazol-4-yl]methylidene]-2-methylpropane-2-sulfinamide (1.5 g, 74.3%) as a yellow solid. LC-MS: (ES+H, m/z) [M+H]⁺=402.1. ¹H NMR (400 MHz, CDCl₃) δ 8.55 (s, 1H), 7.93 (d, J=1.0 Hz, 1H), 6.68-6.64 (m, 1H), 6.60 (d, J=1.7 Hz, 1H), 3.68 (s, 3H), 2.31 (s, 3H), 1.99 (s, 3H), 1.19 (s, 9H).

### Step 3: Preparation of ethyl (3S)-3-[1-(2-methoxy-4,6-dimethylphenyl)-3-(trifluoromethyl)pyrazol-4-yl]-3-{[(R)-2-methylpropane-2-sulfinyl]amino}propanoate

A mixture of Zn (1.22 g, 18.68 mmol, 5.00 equiv.) and TMSCl (0.20 g, 1.86 mmol, 0.50 equiv.) in THF (20 mL) was stirred for 1 h at 60° C. under nitrogen atmosphere. To the above mixture was added ethyl bromoacetate (1.56 g, 9.34 mmol, 2.50 equiv.) dropwise over 10 min at room temperature. The resulting mixture was stirred for an additional 1 h at 60° C. To the above mixture was added (R)—N-[(1Z)-[1-(2-methoxy-4,6-dimethylphenyl)-3-(trifluoromethyl)pyrazol-4-yl]methylidene]-2-methylpropane-2-sulfinamide (1.50 g, 3.73 mmol, 1.00 equiv.) in THF (10 mL) dropwise over 5 min at −20° C. The resulting mixture was stirred for an additional 5 h at −20° C. to 0° C. and then quenched with sat. aq. NH₄Cl (100 mL) at 0° C. The resulting mixture was diluted with EtOAc (100 mL) and then stirred for 10 min at room temperature. The resulting mixture was extracted with EtOAc (2×50 mL) and the combined organic layers were washed with brine (2×20 mL), dried over anhydrous Na₂SO₄, filtered, and concentrated. The residue was purified by silica gel column chromatography (PE/EtOAc (0% to 50% gradient in 30 min)) to afford ethyl (3S)-3-[1-(2-methoxy-4,6-dimethylphenyl)-3-(trifluoromethyl)pyrazol-4-yl]-3-{[(R)-2-methylpropane-2-sulfinyl]amino}propanoate (1.5 g, 82.0%) as a white solid. LC-MS: (ES+H, m/z) [M+H]⁺=490.2. ¹H NMR (400 MHz, DMSO-d₆) δ 7.95 (d, J=1.2 Hz, 1H), 6.90 (d, J=1.8 Hz, 1H), 6.81-6.75 (m, 1H), 5.61 (d, J=7.0 Hz, 1H), 4.80 (q, J=7.2 Hz, 1H), 4.01 (q, J=7.1 Hz, 2H), 3.71 (s, 3H), 3.05 (dd, J=15.4, 6.6 Hz, 1H), 2.87 (dd, J=15.4, 8.2 Hz, 1H), 2.35 (s, 3H), 1.86 (s, 3H), 1.12 (t, J=7.1 Hz, 3H), 1.07 (s, 9H).

### Step 4: Preparation of ethyl (3S)-3-amino-3-[1-(2-methoxy-4,6-dimethylphenyl)-3-(trifluoromethyl)pyrazol-4-yl]propanoate

To a stirred solution of ethyl (3S)-3-[1-(2-methoxy-4,6-dimethylphenyl)-3-(trifluoromethyl)pyrazol-4-yl]-3-{[(R)-2-methylpropane-2-sulfinyl]amino}propanoate (1.50 g, 3.06 mmol, 1.00 equiv.) in CH₂Cl₂ (5 mL) was added HCl in 1,4-dioxane (15 mL, 4 M) dropwise at room temperature under nitrogen atmosphere. The resulting mixture was stirred for 3 h at room temperature and then concentrated under reduced pressure. The residue was purified by reversed-phase flash chromatography (C18 gel; mobile phase, MeCN in water (0.1% NH₃·H₂O), 0% to 70% gradient in 35 min; detector, UV 220 nm) to provide ethyl (3S)-3-amino-3-[1-(2-methoxy-4,6-dimethylphenyl)-3-(trifluoromethyl)pyrazol-4-yl]propanoate (900 mg, 76.2%) as a colorless oil. LC-MS: (ES+H, m/z) [M+H]⁺=386.1. ¹H NMR (400 MHz, DMSO-d₆) δ 7.93 (s, 1H), 6.90 (d, J=1.8 Hz, 1H), 6.78 (d, J=1.7 Hz, 1H), 4.34 (t, J=7.0 Hz, 1H), 4.03 (q, J=7.1 Hz, 2H), 3.71 (s, 3H), 2.64 (dd, J=7.0, 2.1 Hz, 2H), 2.35 (s, 3H), 1.87 (s, 3H), 1.14 (t, J=7.1 Hz, 3H).

### Step 5: Preparation of ethyl (3S)-3-amino-3-[1-(2-hydroxy-4,6-dimethylphenyl)-3-(trifluoromethyl)pyrazol-4-yl]propanoate

To a stirred solution of ethyl (3S)-3-amino-3-[1-(2-methoxy-4,6-dimethylphenyl)-3-(trifluoromethyl)pyrazol-4-yl]propanoate (750 mg, 1.94 mmol, 1.00 equiv.) in CH₂Cl₂ (20 mL) was added boron trichloride (7.80 mL, 7.78 mmol, 4.00 equiv., 1 M in hexane) dropwise at 0° C. under nitrogen atmosphere. The resulting mixture was stirred for 3 h at 0° C. under nitrogen atmosphere. The reaction was quenched by the addition of water (0.5 mL) at 0° C. The resulting mixture was concentrated under reduced pressure. The residue was purified by silica gel column chromatography (CH₂Cl₂/MeOH (0% to 10% gradient in 20 min)) to afford ethyl (3S)-3-amino-3-[1-(2-hydroxy-4,6-dimethylphenyl)-3-(trifluoromethyl)pyrazol-4-yl]propanoate (450 mg, 62.2%) as a brown solid. LC-MS: (ES+H, m/z) [M+H]⁺=372.1. ¹H NMR (400 MHz, DMSO-d₆) δ 10.10 (s, 1H), 8.75 (s, 2H), 8.29 (s, 1H), 6.73 (s, 1H), 6.62 (s, 1H), 4.68 (dd, J=8.7, 5.4 Hz, 1H), 4.02 (q, J=6.9 Hz, 2H), 3.20-3.03 (m, 2H), 2.25 (s, 3H), 1.85 (s, 3H), 1.11 (t, J=6.8 Hz, 3H).

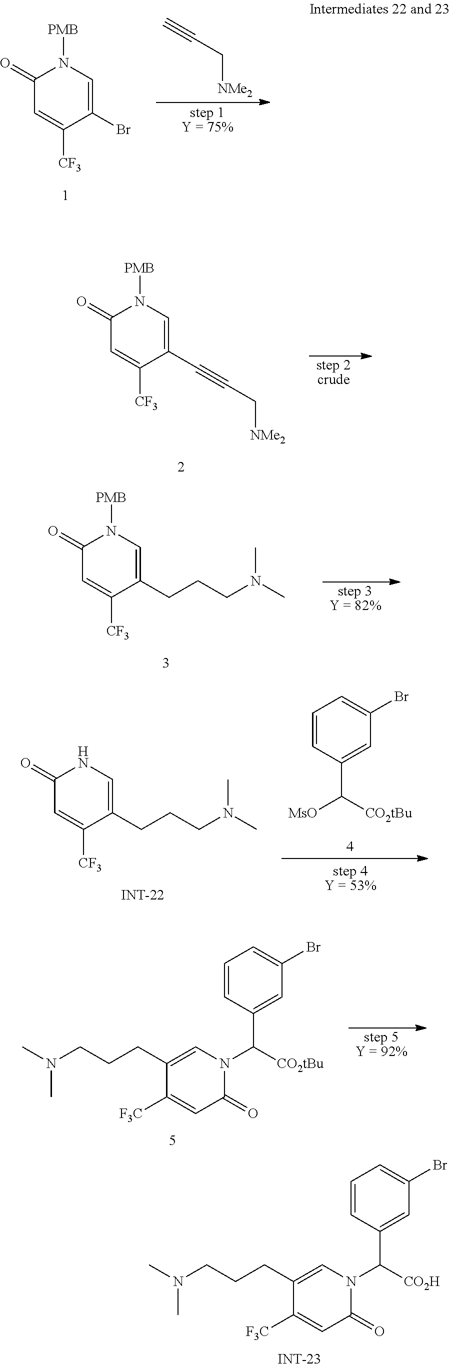

### Step 1: Preparation of 5-[3-(dimethylamino)prop-1-yn-1-yl]-1-[(4-methoxyphenyl)methyl]-4-(trifluoromethyl)pyridin-2-one

A mixture of 5-bromo-1-[(4-methoxyphenyl)methyl]-4-(trifluoromethyl)pyridin-2-one (4.00 g, 11.05 mmol, 1.00 equiv.), dimethyl(prop-2-yn-1-yl)amine (3.67 g, 44.18 mmol, 4.00 equiv.), Pd(PPh₃)₂Cl₂ (775 mg, 1.11 mmol, 0.10 equiv.), CuI (421 mg, 2.21 mmol, 0.20 equiv.) and TEA (22.35 g, 220.90 mmol, 20.00 equiv.) in DME (30 mL) was stirred overnight at 80° C. under nitrogen atmosphere. The mixture was then cooled to room temperature and concentrated. The residue was purified by reversed-phase flash chromatography (C18 gel; mobile phase, MeCN in water (10 mmol/L NH₄HCO₃), 40% to 100% gradient in 30 min; detector, UV 220 nm) to provide 5-[3-(dimethylamino)prop-1-yn-1-yl]-1-[(4-methoxyphenyl)methyl]-4-(trifluoromethyl)pyridin-2-one (3.00 g, 74.5%) as a yellow oil. LC-MS: (ES+H, m/z) [M+H]⁺=365.05. ¹H NMR (300 MHz, CDCl₃) δ 7.56 (s, 1H), 7.31-7.27 (m, 2H), 6.93 (d, J=2.1 Hz, 1H), 6.90 (d, J=1.3 Hz, 2H), 5.06 (s, 2H), 3.82 (s, 3H), 3.43 (s, 2H), 2.33 (s, 6H).

### Step 2: Preparation of 5-[3-(dimethylamino)propyl]-1-[(4-methoxyphenyl)methyl]-4-(trifluoromethyl)pyridin-2-one

To a solution of 5-[3-(dimethylamino)prop-1-yn-1-yl]-1-[(4-methoxyphenyl)methyl]-4-(trifluoromethyl)pyridin-2-one (1.80 g, 4.94 mmol, 1.00 equiv.) in EtOH (150 mL) was added Pd/C (5%, 900 mg) in a pressure tank. The mixture was hydrogenated at room temperature at 30 psi of hydrogen pressure for 2 h, filtered through a Celite pad, and concentrated under reduced pressure. The celite pad was washed with CH₂Cl₂ (3×200 mL) and the combined filtrate was concentrated under reduced pressure to afford 5-[3-(dimethylamino)propyl]-1-[(4-methoxyphenyl)methyl]-4-(trifluoromethyl)pyridin-2-one (1.80 g, crude) as a brown oil. The crude product was used in the next step directly without further purification. LC-MS: (ES+H, m/z) [M+H]⁺=369.1.

### Step 3: Preparation of 5-[3-(dimethylamino)propyl]-4-(trifluoromethyl)-1H-pyridin-2-one

A solution of 5-[3-(dimethylamino)propyl]-1-[(4-methoxyphenyl)methyl]-4-(trifluoromethyl)pyridin-2-one (1.80 g, 3.91 mmol, 1.00 equiv.) and CF₃COOH (30.00 mL) was stirred overnight at 80° C. under nitrogen atmosphere. The mixture was allowed to cool down to room temperature and the residue was purified by reversed-phase flash chromatography (C18 gel; mobile phase, MeCN in water (0.1% NH₃·H₂O), 20% to 60% gradient in 30 min; detector, UV 220 nm) to provide 5-[3-(dimethylamino)propyl]-4-(trifluoromethyl)-1H-pyridin-2-one (1 g, 82.4%) as a white solid. LC-MS: (ES+H, m/z) [M+H]⁺=249.05. ¹H NMR (300 MHz, CDCl₃) δ 7.35 (s, 1H), 6.89 (s, 1H), 2.61-2.52 (m, 2H), 2.34 (dd, J=8.2, 6.4 Hz, 2H), 2.25 (s, 6H), 1.72 (p, J=7.5 Hz, 2H).

### Step 4: Preparation of tert-butyl 2-(3-bromophenyl)-2-{5-[3-(dimethylamino)propyl]-2-oxo-4-(trifluoromethyl)pyridin-1-yl}acetate

A mixture of 5-[3-(dimethylamino)propyl]-4-(trifluoromethyl)-1H-pyridin-2-one (1.00 g, 4.03 mmol, 1.00 equiv.), tert-butyl 2-(3-bromophenyl)-2-(methanesulfonyloxy)acetate (1.54 g, 4.23 mmol, 1.05 equiv.) and K₂CO₃ (1.11 g, 8.06 mmol, 2.00 equiv.) in CH₃CN (30 mL) was stirred for 2 h at 80° C. under nitrogen atmosphere. The resulting mixture was diluted with water (50 mL) and extracted with EtOAc (3×40 mL). The combined organic layers were washed with brine (2×30 mL), dried over anhydrous Na₂SO₄, filtered, and concentrated. The residue was purified by reversed-phase flash chromatography (C18 gel; mobile phase, MeCN in water (0.1% HCOOH), 35% to 65% gradient in 25 min; detector, UV 220 nm) to provide tert-butyl 2-(3-bromophenyl)-2-{5-[3-(dimethylamino)propyl]-2-oxo-4-(trifluoromethyl)pyridin-1-yl}acetate (1.10 g, 52.8%) as a colorless oil. LC-MS: (ES+H, m/z) [M+H]⁺=517.1. ¹H NMR (300 MHz, CDCl₃) δ 7.61-7.51 (m, 2H), 7.39-7.30 (m, 2H), 7.21 (s, 1H), 6.93 (s, 1H), 6.73 (s, 1H), 2.98-2.75 (m, 2H), 2.67 (s, 6H), 2.53 (q, J=6.9 Hz, 2H), 1.87 (s, 2H), 1.50 (s, 9H).

### Step 5: Preparation of (3-bromophenyl)({5-[3-(dimethylamino)propyl]-2-oxo-4-(trifluoromethyl)pyridin-1-yl})acetic acid

A solution of tert-butyl 2-(3-bromophenyl)-2-{5-[3-(dimethylamino)propyl]-2-oxo-4-(trifluoromethyl)pyridin-1-yl}acetate (1.10 g, 2.13 mmol, 1.00 equiv.) and CF₃COOH (15 mL) in CH₂Cl₂ (15 mL) was stirred overnight at room temperature under nitrogen atmosphere. The resulting mixture was concentrated under reduced pressure. The residue was purified by reversed-phase flash chromatography (C18 gel; mobile phase, MeCN in water (0.1% HCOOH), 0% to 40% gradient in 30 min; detector, UV 220 nm) to provide (3-bromophenyl)({5-[3-(dimethylamino)propyl]-2-oxo-4-(trifluoromethyl)pyridin-1-yl})acetic acid (900 mg, 91.8%) as a colorless oil. LC-MS: (ES+H, m/z) [M+H]⁺=461.0.

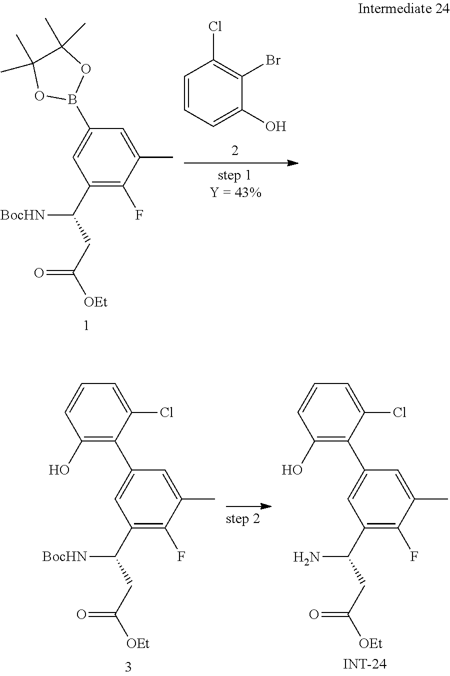

### Step 1: Preparation of ethyl (3S)-3-[(tert-butoxycarbonyl)amino]-3-{2′-chloro-4-fluoro-6′-hydroxy-5-methyl-[1,1′-biphenyl]-3-yl}propanoate

To a stirred solution of 2-bromo-3-chlorophenol (505 mg, 2.43 mmol, 1.10 equiv.) and ethyl (3S)-3-[(tert-butoxycarbonyl)amino]-3-[2-fluoro-3-methyl-5-(4,4,5,5-tetramethyl-1,3,2-dioxaborolan-2-yl)phenyl]propanoate (1.00 g, 2.21 mmol, 1.00 equiv.) in 1,4-dioxane (15.00 mL) and H₂O (0.75 mL) was added K₂CO₃ (918 mg, 6.64 mmol, 3.00 equiv.) and Pd(dppf)Cl₂CH₂Cl₂ (180 mg, 0.22 mmol, 0.10 equiv.) at room temperature under nitrogen atmosphere. The resulting mixture was stirred for 3 h at 100° C. under nitrogen atmosphere. The mixture was allowed to cool down to room temperature and concentrated under reduced pressure. The residue was purified by silica gel column chromatography (PE/EtOAc (0% to 50% gradient in 30 min)) to afford ethyl (3S)-3-[(tert-butoxycarbonyl)amino]-3-{2′-chloro-4-fluoro-6′-hydroxy-5-methyl-[1,1′-biphenyl]-3-yl}propanoate (450 mg, 42.7%) as a yellow solid. LC-MS: (ES+H, m/z) [M+H]⁺=452.2. ¹H NMR (400 MHz, CD₃OD) δ 7.14-7.05 (m, 2H), 7.03 (dd, J=7.2, 2.2 Hz, 1H), 6.94 (dd, J=8.1, 1.1 Hz, 1H), 6.81 (dd, J=8.2, 1.1 Hz, 1H), 5.36 (t, J=7.4 Hz, 1H), 4.12-4.09 (m, 2H), 2.78-2.75 (m, 2H), 2.30 (d, J=2.1 Hz, 3H), 1.40 (s, 9H), 1.20 (t, J=7.1 Hz, 3H).

### Step 2: Preparation of ethyl (3S)-3-amino-3-{2′-chloro-4-fluoro-6′-hydroxy-5-methyl-[1,1′-biphenyl]-3-yl}propanoate hydrochloride

To a stirred solution of ethyl (3S)-3-[(tert-butoxycarbonyl)amino]-3-{2′-chloro-4-fluoro-6′-hydroxy-5-methyl-[1,1′-biphenyl]-3-yl}propanoate (440 mg, 0.97 mmol, 1.00 equiv.) in CH₂Cl₂ (2.50 mL) was added HCl in 1,4-dioxane (2.50 mL, 4 M) at room temperature under nitrogen atmosphere. The resulting mixture was stirred for 2 h and then concentrated under reduced pressure. The crude product, ethyl (3S)-3-amino-3-{2′-chloro-4-fluoro-6′-hydroxy-5-methyl-[1,1′-biphenyl]-3-yl}propanoate hydrochloride (450 mg, crude), was used in the next step directly without further purification. LC-MS: (ES+H, m/z) [M+H]⁺=352.2.

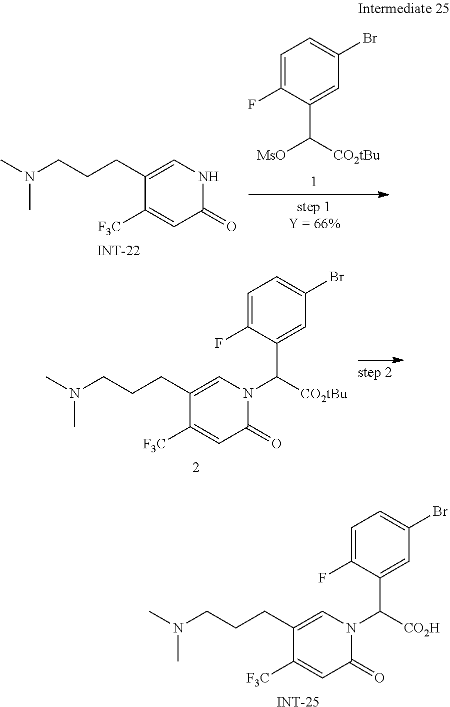

### Step 1: Preparation of tert-butyl 2-(5-bromo-2-fluorophenyl)-2-{5-[3-(dimethylamino)propyl]-2-oxo-4-(trifluoromethyl)pyridin-1-yl}acetate

To a stirred solution of 5-[3-(dimethylamino)propyl]-4-(trifluoromethyl)-1H-pyridin-2-one (350 mg, 1.41 mmol, 1.00 equiv.) and tert-butyl 2-(5-bromo-2-fluorophenyl)-2-(methanesulfonyloxy)acetate (540 mg, 1.41 mmol, 1.00 equiv.) in MeCN (10 mL) were added K₂CO₃ (389 mg, 2.82 mmol, 2.00 equiv.) at room temperature under nitrogen atmosphere. The resulting mixture was heated for 2 h at 80° C. and then cooled to room temperature and diluted with water. The resulting mixture was extracted with EtOAc (4×50 mL). The combined organic layers were washed with brine (1×10 mL), dried over anhydrous Na₂SO₄, filtered, and concentrated. The residue was purified by reversed-phase flash chromatography (C18 gel; mobile phase, MeCN in water (0.1% NH₃·H₂O), 40% to 80% gradient in 20 min; detector, UV 254 nm) to provide tert-butyl 2-(5-bromo-2-fluorophenyl)-2-{5-[3-(dimethylamino)propyl]-2-oxo-4-(trifluoromethyl)pyridin-1-yl}acetate (500 mg, 66.2%) as light yellow oil. LC-MS: (ES+H, m/z) [M+H]⁺=537.05. ¹H NMR (300 MHz, CDCl₃) δ 7.60-7.50 (m, 2H), 7.13 (s, 1H), 7.05 (dd, J=9.6, 8.7 Hz, 1H), 6.91 (s, 1H), 6.77 (s, 1H), 2.48 (dd, J=8.9, 6.4 Hz, 2H), 2.35-2.20 (m, 2H), 2.17 (s, 6H), 1.69-1.56 (m, 2H), 1.49 (s, 9H). ¹⁹F NMR (282 MHz, CDCl₃) δ −63.96, −116.36.

### Step 2: Preparation of (5-bromo-2-fluorophenyl)({5-[3-(dimethylamino)propyl]-2-oxo-4-(trifluoromethyl)pyridin-1-yl})acetic acid

To a stirred solution of tert-butyl 2-(5-bromo-2-fluorophenyl)-2-{5-[3-(dimethylamino)propyl]-2-oxo-4-(trifluoromethyl)pyridin-1-yl}acetate (430 mg, 0.80 mmol, 1.00 equiv.) in CH₂Cl₂ (2.5 mL) was added CF₃COOH (5 mL) dropwise at 0° C. under nitrogen atmosphere. The resulting mixture was stirred for 2 h at room temperature and then concentrated under reduced pressure to provide (5-bromo-2-fluorophenyl)({5-[3-(dimethylamino)propyl]-2-oxo-4-(trifluoromethyl)pyridin-1-yl})acetic acid (680 mg, crude) as a light brown oil. The crude product mixture was used in the next step directly without further purification. LC-MS: (ES+H, m/z) [M+H]⁺=479.10.

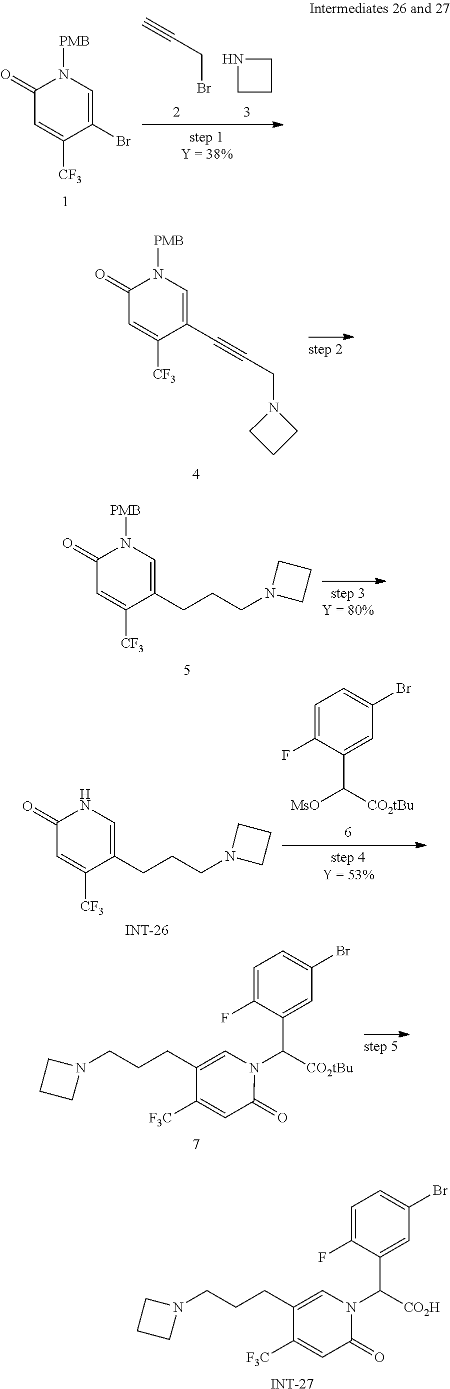

### Step 1: Preparation of 5-(3-(azetidin-1-yl) prop-1-yn-1-yl)-1-(4-methoxybenzyl)-4-(trifluoromethyl) pyridin-2(1H)-one

To a stirred mixture of propargyl bromide (4.93 g, 41.42 mmol, 3.00 equiv.) and azetidine (4.73 g, 82.84 mmol, 6.00 equiv.) in DMF (30 mL) was added K₂CO₃ (11.45 g, 82.84 mmol, 6.00 equiv.) at room temperature under nitrogen atmosphere. The resulting mixture was stirred for 4 h at 100° C. To the above mixture was added CuI (0.53 g, 2.76 mmol, 0.20 equiv.), Pd(PPh₃)₂Cl₂ (0.97 g, 1.38 mmol, 0.10 equiv.) triethylamine (38 mL, 276.14 mmol, 20.00 equiv.) and DME (100 mL) at room temperature. The resulting mixture was stirred overnight at 100° C., cooled to room temperature, and filtered. The filter cake was washed with CH₂Cl₂ (3×20 mL) and the combined filtrates were concentrated under reduced pressure. The residue was purified by reversed-phase flash chromatography (C18; mobile phase, MeCN in water (10 mmol/L NH₄HCO₃), 10% to 50% gradient in 20 min; detector, UV 220 nm) to afford 5-[3-(azetidin-1-yl) prop-1-yn-1-yl]-1-[(4-methoxyphenyl) methyl]-4-(trifluoromethyl) pyridin-2-one (2.00 g, 38.4%) as a yellow oil. LC-MS: (ES+H, m/z) [M+H]⁺=377.10. ¹H NMR (400 MHz, CDCl₃) δ 7.68 (s, 1H), 7.31-7.28 (m, 2H), 6.93-6.90 (m, 3H), 5.07 (s, 2H), 3.95 (t, J=8.0 Hz, 4H), 3.81 (s, 5H), 2.44-2.34 (m, 2H). ¹⁹F NMR (400 MHz, CDCl₃) δ −65.83.

### Step 2: Preparation of 5-(3-(azetidin-1-yl) propyl)-1-(4-methoxybenzyl)-4-(trifluoromethyl) pyridin-2(1H)-one

A mixture of 5-[3-(azetidin-1-yl) prop-1-yn-1-yl]-1-[(4-methoxyphenyl) methyl]-4-(trifluoromethyl) pyridin-2-one (2.00 g, 5.31 mmol, 1.00 equiv.) and Pd/C (1.00 g, wt 10%) in EtOAc (50 mL) was stirred for 3 h at room temperature under hydrogen atmosphere. The reaction mixture was then filtered and the filter cake was washed with CH₂Cl₂ (3×100 mL). The combined filtrate was concentrated under reduced pressure and the crude product was purified by silica gel column chromatography (CH₂Cl₂/MeOH (0 to 15% gradient in 30 min)) to afford 5-[3-(azetidin-1-yl) propyl]-1-[(4-methoxyphenyl) methyl]-4-(trifluoromethyl) pyridin-2-one (750 mg, 37.1%) as a yellow oil. LC-MS: (ES+H, m/z) [M+H]⁺=381.15.

### Step 3: Preparation of 5-(3-(azetidin-1-yl) propyl)-4-(trifluoromethyl) pyridin-2(1H)-one

A solution of 5-[3-(azetidin-1-yl) propyl]-1-[(4-methoxyphenyl) methyl]-4-(trifluoromethyl) pyridin-2-one (1.10 g, 2.89 mmol, 1.00 equiv.) in TFA (10 mL) was stirred overnight at 80° C. under nitrogen atmosphere. The resulting mixture was then cooled to room temperature and concentrated under reduced pressure. The residue was purified by reversed-phase flash chromatography (C18; mobile phase, MeCN in water (10 mmol/L NH₄HCO₃), 10% to 50% gradient in 20 min; detector, UV 220 nm) to afford 5-[3-(azetidin-1-yl) propyl]-4-(trifluoromethyl)-1H-pyridin-2-one (600 mg, 79.7%) as a yellow solid. LC-MS: (ES+H, m/z) [M+H−tBu]⁺=261.20. ¹H NMR (400 MHz, DMSO-d₆) δ 7.46 (s, 1H), 6.67 (s, 1H), 3.09 (t, J=7.0 Hz, 4H), 2.47-2.39 (m, 2H), 2.36 (t, J=6.9 Hz, 2H), 2.00-1.90 (m, 2H), 1.45 (p, J=7.1 Hz, 2H). ¹⁹F NMR (377 MHz, DMSO-d₆) δ −62.71.

### Step 4: Preparation of tert-butyl 2-{5-[3-(azetidin-1-yl) propyl]-2-oxo-4-(trifluoromethyl) pyridin-1-yl}-2-(5-bromo-2-fluorophenyl) acetate

To a stirred solution of 5-[3-(azetidin-1-yl) propyl]-4-(trifluoromethyl)-1H-pyridin-2-one (400 mg, 1.53 mmol, 1.00 equiv.) and tert-butyl 2-(5-bromo-2-fluorophenyl)-2-(methanesulfonyloxy) acetate (589 mg, 1.53 mmol, 1.00 equiv.) in MeCN (15 mL) was added K₂CO₃ (424 mg, 3.07 mmol, 2.00 equiv.) at room temperature under nitrogen atmosphere. The resulting mixture was stirred overnight at 80° C. under nitrogen atmosphere. The resulting mixture was concentrated under reduced pressure. The residue was purified by reversed-phase flash chromatography (C18; mobile phase, MeOH in water (10 mmol/L NH₄HCO₃), 10% to 50% gradient in 20 min; detector, UV 220 nm) to afford tert-butyl 2-{5-[3-(azetidin-1-yl) propyl]-2-oxo-4-(trifluoromethyl) pyridin-1-yl}-2-(5-bromo-2-fluorophenyl)acetate (450 mg, 53.4%) as a colorless oil. LC-MS: (ES+H, m/z) [M+H]⁺=547.15. ¹H NMR (400 MHz, CDCl₃) δ7.60-7.50 (m, 2H), 7.13-7.01 (m, 2H), 6.90 (s, 1H), 6.77 (s, 1H), 3.11 (t, J=7.0 Hz, 4H), 2.49-2.26 (m, 4H), 2.07-2.01 (m, 2H), 1.57-1.44 (m, 11H). ¹⁹F NMR (377 MHz, CDCl₃) δ −64.00, −116.34.

### Step 5: Preparation of 2-(5-(3-(azetidin-1-yl) propyl)-2-oxo-4-(trifluoromethyl) pyridin-1(2H)-yl)-2-(5-bromo-2-fluorophenyl) acetic acid

To a stirred solution of tert-butyl 2-{5-[3-(azetidin-1-yl) propyl]-2-oxo-4-(trifluoromethyl) pyridin-1 yl}-2-(5-bromo-2-fluorophenyl) acetate (400 mg, 0.73 mmol, 1.00 equiv.) in CH₂Cl₂ (2 mL) was added CF₃COOH (4 mL) at room temperature under nitrogen atmosphere. The resulting mixture was stirred overnight at room temperature under nitrogen atmosphere and then concentrated under reduced pressure. The residue was purified by trituration with Et₂O (20 mL) to afford {5-[3-(azetidin-1-yl) propyl]-2-oxo-4-(trifluoromethyl) pyridin-1-yl} (5-bromo-2-fluorophenyl) acetic acid (650 mg, crude) as an off-white solid. LC-MS: (ES+H, m/z) [M+H]⁺=491.0.

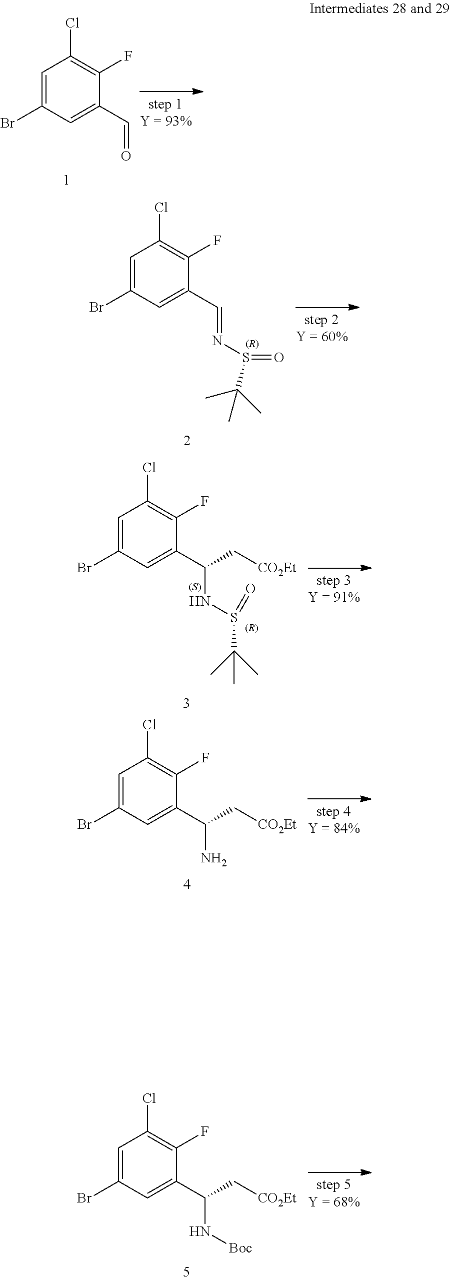

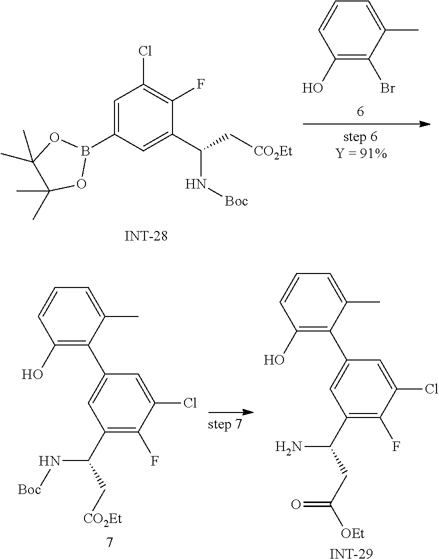

### Step 1: Preparation of (R)—N-[(1E)-(5-bromo-3-chloro-2-fluorophenyl)methylidene]-2-methylpropane-2-sulfinamide

A solution of 5-bromo-3-chloro-2-fluorobenzaldehyde (3.00 g, 12.63 mmol, 1.00 equiv.), (R)-2-methylpropane-2-sulfinamide (1.68 g, 13.90 mmol, 1.10 equiv.) and Ti(Oi-Pr)₄ (7.18 g, 25.27 mmol, 2.00 equiv.) in THF (50 mL) was stirred for 4 h at room temperature under nitrogen atmosphere. The resulting mixture was diluted with water (100 mL) and EtOAc (100 mL). The resulting mixture was extracted with EtOAc (3×100 mL). The combined organic layers were washed with brine (3×100 mL), dried over anhydrous Na₂SO₄, filtered, and concentrated. The residue was purified by silica gel column chromatography (PE/EA (0%˜40% in 20 min) to afford (R)—N-[(1E)-(5-bromo-3-chloro-2-fluorophenyl)methylidene]-2-methylpropane-2-sulfinamide (4.00 g, 92.9%) as a white solid. LC-MS: (ES+H, m/z) [M+H]⁺=339.9. ¹H NMR (300 MHz, DMSO-d₆) δ 8.60 (s, 1H), 8.15 (dd, J 6.5, 2.5 Hz, 1H), 8.07 (dd, J 5.6, 2.5 Hz, 1H), 1.20 (s, 9H). ¹⁹F NMR (282 MHz, DMSO-d₆) δ −121.85.

### Step 2: Preparation of ethyl (3S)-3-(5-bromo-3-chloro-2-fluorophenyl)-3-{[(R)-2-methylpropane-2-sulfinyl]amino}propanoate

A mixture of Zn (1.34 g, 20.50 mmol, 4.99 equiv.) and TMSCl (111 mg, 1.02 mmol, 0.25 equiv.) in THF (10 mL) was stirred for 1 h at 60° C. under nitrogen atmosphere. The mixture was allowed to cool down to room temperature. To the above mixture was added ethyl 2-bromoacetate (1.72 g, 10.29 mmol, 2.51 equiv.) dropwise over 10 min at room temperature. The resulting mixture was stirred for an additional 1 h at 60° C. The mixture was allowed to cool down to 0° C. To the above mixture was added (R)—N-[(1E)-(5-bromo-3-chloro-2-fluorophenyl)methylidene]-2-methylpropane-2-sulfinamide (1.40 g, 4.11 mmol, 1.00 equiv.) solution of THF (5 mL) dropwise over 15 min at 0° C. The resulting mixture was stirred for an additional 1 h at 0° C. and then was filtered. The filter cake was washed with EtOAc and the combined filtrates were concentrated under reduced pressure. The residue was purified by silica gel column chromatography (EtOAc/petroleum ether (20%-50% in 25 min) to afford ethyl (3S)-3-(5-bromo-3-chloro-2-fluorophenyl)-3-{[(R)-2-methylpropane-2-sulfinyl]amino}propanoate (1.05 g, 59.5%) as a yellow oil. LC-MS: (ES+H, m/z) [M+H]⁺=427.9.

### Step 3: Preparation of ethyl (3S)-3-amino-3-(5-bromo-3-chloro-2-fluorophenyl)propanoate

A solution of ethyl (3S)-3-(5-bromo-3-chloro-2-fluorophenyl)-3-{[(R)-2-methylpropane-2-sulfinyl]amino}propanoate (1.00 g, 2.33 mmol, 1.00 equiv.) in CH₂Cl₂ (5 mL) was treated with HCl (gas) in 1,4-dioxane (5 mL, 4 M) for 5 min at room temperature under nitrogen atmosphere. The resulting mixture was stirred for 1 h at room temperature under nitrogen atmosphere and then concentrated. The residue was purified by reversed-phase flash chromatography (C18; mobile phase, MeCN in water (10 mmol/L NH₄HCO₃), 20% to 50% gradient in 25 min; detector, UV 220 nm) to afford ethyl (3S)-3-amino-3-(5-bromo-3-chloro-2-fluorophenyl)propanoate (690 mg, 91.2%) as a yellow oil. LC-MS: (ES+H, m/z) [M+H]⁺=323.9. ¹H NMR (400 MHz, DMSO-d₆) δ 7.80-7.70 (m, 2H), 4.45 (t, J=7.6, 6.4 Hz, 1H), 4.02 (q, J=7.1 Hz, 2H), 2.61 (dd, J=7.0, 2.9 Hz, 2H), 2.24 (s, 2H), 1.12 (t, J=7.1 Hz, 3H). ¹⁹F NMR (400 MHz, DMSO-d₆) −123.14.

### Step 4: Preparation of ethyl (3S)-3-(5-bromo-3-chloro-2-fluorophenyl)-3-[(tert-butoxycarbonyl)amino]propanoate

To a stirred mixture of ethyl (3S)-3-amino-3-(5-bromo-3-chloro-2-fluorophenyl)propanoate (528 mg, 1.62 mmol, 1.00 equiv.) and Boc₂O (532 mg, 2.44 mmol, 1.50 equiv.) in CH₂Cl₂ (8 mL) was added DIEA (630 mg, 4.88 mmol, 3.00 equiv.) dropwise at room temperature under nitrogen atmosphere. The resulting mixture was stirred for 2 h at room temperature and then concentrated. The residue was purified by silica gel column chromatography (EtOAc/petroleum ether (20˜50% in 25 min) to afford ethyl (3S)-3-(5-bromo-3-chloro-2-fluorophenyl)-3-[(tert-butoxycarbonyl)amino]propanoate (580 mg, 83.9%) as a yellow oil. LC-MS: (ES−H, m/z) [M−H]⁻=422.1.

### Step 5: Preparation of ethyl (3S)-3-[(tert-butoxycarbonyl)amino]-3-[3-chloro-2-fluoro-5-(4,4,5,5-tetramethyl-1,3,2-dioxaborolan-2-yl)phenyl]propanoate

To a stirred mixture of ethyl (3S)-3-(5-bromo-3-chloro-2-fluorophenyl)-3-[(tert-butoxycarbonyl)amino]propanoate (580 mg, 1.36 mmol, 1.00 equiv.) and bis(pinacolato)diboron (416 mg, 1.63 mmol, 1.20 equiv.) in 1,4-dioxane (8 mL) were added Pd(dppf)Cl₂·CH₂Cl₂ (55 mg, 0.06 mmol, 0.05 equiv.) and KOAc (268 mg, 2.73 mmol, 2.00 equiv.) in portions at room temperature under nitrogen atmosphere. The resulting mixture was stirred for 3 h at 100° C. under nitrogen atmosphere and then cooled to room temperature. The resulting mixture was filtered and the filter cake was washed with EtOAc. The filtrate was concentrated under reduced pressure. The residue was purified by silica gel column chromatography (EtOAc/petroleum ether (30˜60% in 25 min) to afford ethyl (3S)-3-[(tert-butoxycarbonyl)amino]-3-[3-chloro-2-fluoro-5-(4,4,5,5-tetramethyl-1,3,2-dioxaborolan-2-yl)phenyl]propanoate (415 mg, 64.4%) as a yellow oil. LC-MS: (ES+H, m/z) [M+H]⁺=472.2.

### Step 6: Preparation of ethyl (3S)-3-[(tert-butoxycarbonyl)amino]-3-{5-chloro-4-fluoro-2′-hydroxy-6′-methyl-[1,1′-biphenyl]-3-yl}propanoate

To a stirred mixture of ethyl (3S)-3-[(tert-butoxycarbonyl)amino]-3-[3-chloro-2-fluoro-5-(4,4,5,5-tetramethyl-1,3,2-dioxaborolan-2-yl)phenyl]propanoate (415 mg, 0.88 mmol, 1.00 equiv.) and 2-bromo-3-methylphenol (197 mg, 1.05 mmol, 1.20 equiv.) in 1,4-dioxane (5 mL) were added Pd(dppf)Cl₂·CH₂Cl₂ (35 mg, 0.04 mmol, 0.05 equiv.) and K₂CO₃ (243 mg, 1.76 mmol, 2.00 equiv.) in portions at room temperature under nitrogen atmosphere. The resulting mixture was stirred for 3 h at 100° C. under nitrogen atmosphere and then cooled to room temperature. The resulting mixture was filtered, and the filter cake was washed with EtOAc, and the combined filtrates were concentrated under reduced pressure. The residue was purified by silica gel column chromatography (PE/EA (20%˜50% in 25 min) to afford ethyl (3S)-3-[(tert-butoxycarbonyl)amino]-3-{5-chloro-4-fluoro-2′-hydroxy-6′-methyl-[1,1′-biphenyl]-3-yl}propanoate (360 mg, 90.5%) as a yellow oil. LC-MS: (ES−H, m/z) [M−H]⁻=450.1. ¹H NMR (400 MHz, DMSO-d₆) δ 9.28 (s, 1H), 7.60 (d, J=8.7 Hz, 1H), 7.30-7.17 (m, 2H), 7.07 (t, J=7.8 Hz, 1H), 6.75 (dd, J=12.5, 7.8 Hz, 2H), 5.27 (q, J=8.0 Hz, 1H), 4.07-4.01 (m, 2H), 2.72 (d, J=7.4 Hz, 2H), 1.97 (s, 3H), 1.34 (s, 9H), 1.13 (t, J=7.1 Hz, 3H). ¹⁹F NMR (400 MHz, DMSO-d₆) −125.31.

### Step 7: Preparation of ethyl (3S)-3-amino-3-{5-chloro-4-fluoro-2′-hydroxy-6′-methyl-[1,1′-biphenyl]-3-yl}propanoate hydrochloride

A solution of ethyl (3S)-3-[(tert-butoxycarbonyl)amino]-3-{5-chloro-4-fluoro-2′-hydroxy-6′-methyl-[1,1′-biphenyl]-3-yl}propanoate (360 mg, 0.79 mmol, 1.00 equiv.) in CH₂Cl₂ (2 mL) was treated with HCl (gas) in 1,4-dioxane (2 mL, 4 M) for 3 min at room temperature under nitrogen atmosphere. The resulting mixture was stirred for 2 h at room temperature under nitrogen atmosphere and then concentrated under vacuum to afford ethyl (3S)-3-amino-3-{5-chloro-4-fluoro-2′-hydroxy-6′-methyl-[1,1′-biphenyl]-3-yl}propanoate hydrochloride (300 mg, crude) as a yellow crude oil. The crude product was used in the next step directly without further purification. LC-MS: (ES+H, m/z) [M+H]⁺=352.05.

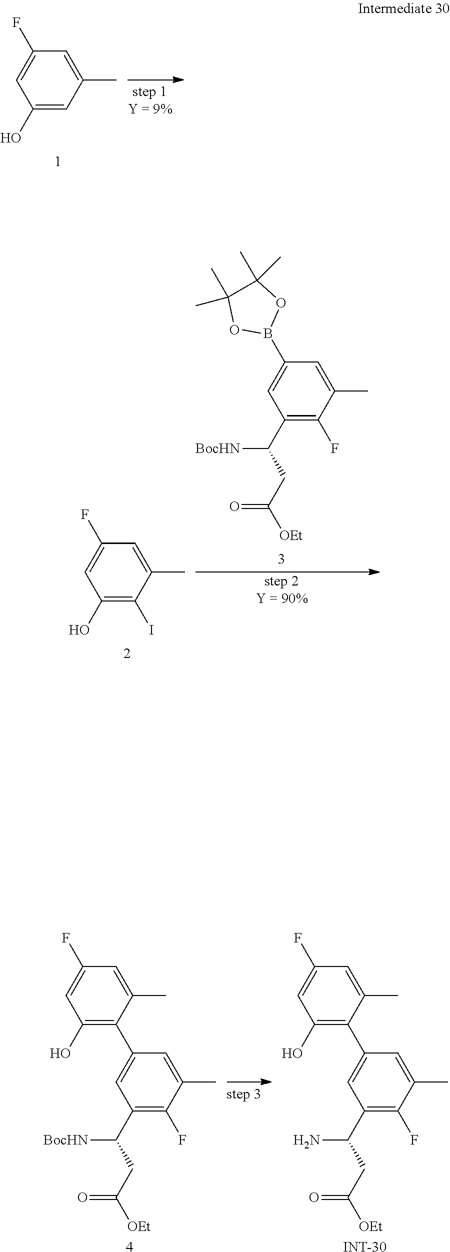

### Step 1: Preparation of 5-fluoro-2-iodo-3-methylphenol

To a stirred mixture of 3-fluoro-5-methylphenol (5.00 g, 39.64 mmol, 1.00 equiv.) in PhCH₃ (50 mL) was added NaH (3.17 g, 79.28 mmol, 2.00 equiv., 60% in mineral oil) at 0° C. under nitrogen atmosphere. The resulting mixture was stirred for 30 min at room temperature and then I₂ (10.06 g, 39.64 mmol, 1.00 equiv.) was added dropwise. The resulting mixture was stirred for an additional 3 h at room temperature and then diluted with water (150 mL). The mixture was acidified to pH 3 with 3 M HCl (aq.) and extracted with EtOAc (2×200 mL). The combined organic layers were washed with brine (1×300 mL), dried over anhydrous Na₂SO₄, filtered, and concentrated. The residue was purified by silica gel column chromatography (PE/EtOAc (0% to 20% gradient in 30 min)) to afford 5-fluoro-2-iodo-3-methylphenol (900 mg, 9%) as a white solid. ¹H NMR (400 MHz, DMSO-d₆) δ 10.73 (s, 1H), 6.71 (ddd, J=9.6, 2.9, 0.8 Hz, 1H), 6.53 (dd, J=10.5, 2.9 Hz, 1H), 2.36 (s, 3H).

### Step 2: Preparation of ethyl (3S)-3-[(tert-butoxycarbonyl)amino]-3-{4,4′-difluoro-2′-hydroxy-5,6′-dimethyl-[1,1′-biphenyl]-3-yl}propanoate

To a stirred mixture of ethyl (3S)-3-[(tert-butoxycarbonyl)amino]-3-[2-fluoro-3-methyl-5-(4,4,5,5-tetramethyl-1,3,2-dioxaborolan-2-yl)phenyl]propanoate (500 mg, 1.10 mmol, 1.00 equiv.) and 5-fluoro-2-iodo-3-methylphenol (307 mg, 1.21 mmol, 1.10 equiv.) in dioxane (10 mL) and H₂O (2 mL) were added Pd(dppf)Cl₂ (81 mg, 0.11 mmol, 0.10 equiv.) and K₂CO₃ (459 mg, 3.32 mmol, 3.00 equiv.) at room temperature under nitrogen atmosphere. The resulting mixture was stirred for 2 h at 100° C. under nitrogen atmosphere. The resulting mixture was filtered and the filter cake was washed with EtOAc (3×100 mL). The filtrate was concentrated under reduced pressure. The residue was purified by silica gel column chromatography (PE/EtOAc (0% to 30% gradient in 30 min)) to afford ethyl (3S)-3-[(tert-butoxycarbonyl)amino]-3-{4,4′-difluoro-2′-hydroxy-5,6′-dimethyl-[1,1′-biphenyl]-3-yl}propanoate (450 mg, 90.3%) as a white solid. LC-MS: (ES+H, m/z) [M+H]⁺=450.2. ¹H NMR (300 MHz, DMSO-d₆) δ 9.64 (s, 1H), 7.51 (d, J=8.9 Hz, 1H), 7.02 (d, J=6.5 Hz, 1H), 6.95 (dd, J=7.2, 2.2 Hz, 1H), 6.61-6.50 (m, 2H), 5.27 (d, J=8.4 Hz, 1H), 4.04 (ttd, J=7.1, 4.5, 2.3 Hz, 2H), 2.65 (d, J=7.4 Hz, 2H), 2.24 (d, J=2.0 Hz, 3H), 1.95 (s, 3H), 1.34 (s, 9H), 1.14 (t, J=7.1 Hz, 3H).

### Step 3: Preparation of ethyl (3S)-3-amino-3-{4,4′-difluoro-2′-hydroxy-5,6′-dimethyl-[1,1′-biphenyl]-3-yl}propanoate hydrochloride

To a stirred mixture of ethyl (3S)-3-[(tert-butoxycarbonyl)amino]-3-{4,4′-difluoro-2′-hydroxy-5,6′-dimethyl-[1,1′-biphenyl]-3-yl}propanoate (350 mg, 0.77 mmol, 1.00 equiv.) in CH₂C₂ (2 mL) was added HCl (gas) in 1,4-dioxane (4 mL, 4 M) at room temperature under nitrogen atmosphere. The resulting mixture was stirred for 1 h at room temperature under nitrogen atmosphere and then concentrated to afford ethyl (3S)-3-amino-3-{4,4′-difluoro-2′-hydroxy-5,6′-dimethyl-[1,1′-biphenyl]-3-yl}propanoate hydrochloride (350 mg, crude) as a white solid. LC-MS: (ES+H, m/z) [M+H]⁺=350.1.

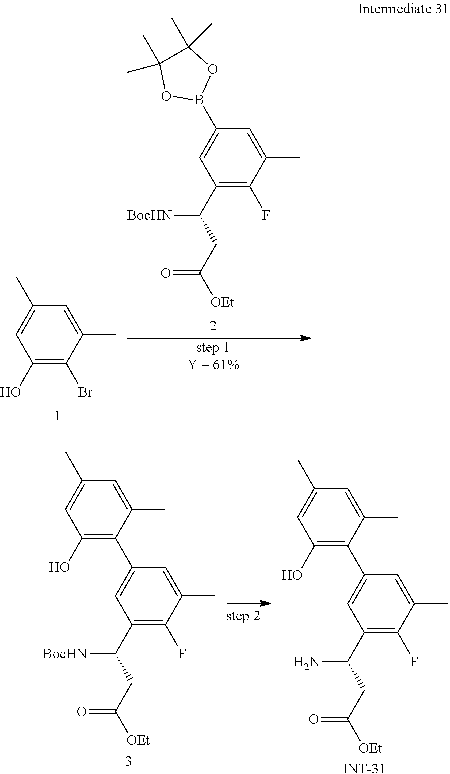

### Step 1: Preparation of ethyl (3S)-3-[(tert-butoxycarbonyl)amino]-3-{4-fluoro-2′-hydroxy-4′,5,6′-trimethyl-[1,1′-biphenyl]-3-yl}propanoate

To a stirred mixture of ethyl (3S)-3-[(tert-butoxycarbonyl)amino]-3-[2-fluoro-3-methyl-5-(4,4,5,5-tetramethyl-1,3,2-dioxaborolan-2-yl)phenyl]propanoate (500 mg, 1.11 mmol, 1.00 equiv.) and 2-bromo-3,5-dimethylphenol (245 mg, 1.22 mmol, 1.10 equiv.) in dioxane (10 mL) and H₂O (0.5 mL) were added Pd(dppf)Cl₂ (81 mg, 0.11 mmol, 0.10 equiv.) and K₂CO₃ (459 mg, 3.32 mmol, 3.00 equiv.) at room temperature under nitrogen atmosphere. The resulting mixture was stirred for 2 h at 100° C. under nitrogen atmosphere and then cooled to room temperature. The resulting mixture was concentrated under reduced pressure. The residue was purified by silica gel column chromatography (PE/EtOAc (0 to 20% gradient in 30 min)) to afford ethyl (3S)-3-[(tert-butoxycarbonyl)amino]-3-{4-fluoro-2′-hydroxy-4′,5,6′-trimethyl-[1,1′-biphenyl]-3-yl}propanoate (300 mg, 60.8%) as a light yellow solid. LC-MS: (ES+H, m/z) [M+H−tBu]⁺=390.1. ¹H NMR (300 MHz, DMSO-d₆) δ 8.96 (s, 1H), 7.51 (d, J=8.9 Hz, 1H), 7.01 (d, J=6.7 Hz, 1H), 6.93 (d, J=7.2 Hz, 1H), 6.55 (d, J=5.3 Hz, 2H), 5.27 (d, J=8.0 Hz, 1H), 4.08-4.00 (m, 2H), 2.65 (d, J=7.3 Hz, 2H), 2.26-2.19 (m, 6H), 1.91 (s, 3H), 1.34 (s, 9H), 1.14 (t, J=7.1 Hz, 3H). ¹⁹F NMR (282 MHz, DMSO-d₆) δ −127.73.

### Step 2: Preparation of ethyl (3S)-3-amino-3-{4-fluoro-2′-hydroxy-4′,5,6′-trimethyl-[1,1′-biphenyl]-3-yl}propanoate hydrochloride

To a stirred mixture of ethyl (3S)-3-[(tert-butoxycarbonyl)amino]-3-{4-fluoro-2′-hydroxy-4′,5,6′-trimethyl-[1,1′-biphenyl]-3-yl}propanoate (280 mg, 0.63 mmol, 1.00 equiv.) in CH₂Cl₂ (5 mL) was added HCl (gas) in 1,4-dioxane (5 mL, 4 M) dropwise at room temperature under nitrogen atmosphere. The resulting mixture was stirred for 2 h at room temperature under nitrogen atmosphere and then concentrated to provide ethyl (3S)-3-amino-3-{4-fluoro-2′-hydroxy-4′,5,6′-trimethyl-[1,1′-biphenyl]-3-yl}propanoate hydrochloride (260 mg, crude) as a light yellow oil. LC-MS: (ES+H, m/z) [M+H]⁺=346.1.

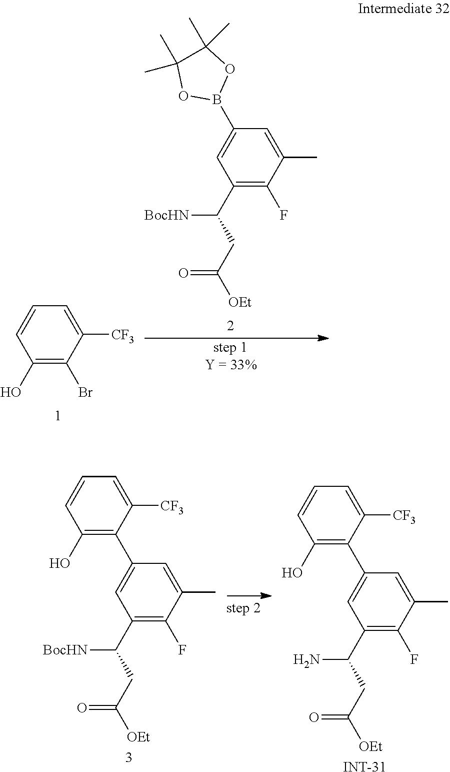

### Step 1: Preparation of ethyl (3S)-3-[(tert-butoxycarbonyl)amino]-3-[4-fluoro-2′-hydroxy-5-methyl-6′-(trifluoromethyl)-[1,1′-biphenyl]-3-yl]propanoate

To a stirred solution of 2-bromo-3-(trifluoromethyl)phenol (0.53 g, 2.22 mmol, 1.00 equiv.) and ethyl (3S)-3-[(tert-butoxycarbonyl)amino]-3-[2-fluoro-3-methyl-5-(4,4,5,5-tetramethyl-1,3,2-dioxaborolan-2-yl)phenyl]propanoate (1.00 g, 2.21 mmol, 1.00 equiv.) in dioxane (10 mL) and water (1 mL) were added Pd(dppf)Cl₂ CH₂Cl₂ (0.09 g, 0.11 mmol, 0.05 equiv.) and KH₂PO₄ (0.9 g, 6.65 mmol, 3.00 equiv.) dropwise at room temperature under nitrogen atmosphere. The resulting mixture was stirred overnight at 90° C. under nitrogen atmosphere and then cooled to room temperature. The resulting mixture was diluted with EtOAc (200 mL). The residue was washed with water (2×20 mL), dried over anhydrous Na₂SO₄, filtered, and concentrated. The residue was purified by silica gel column chromatography (PE/EtOAc (0% to 30% gradient in 30 min)) to afford ethyl (3S)-3-[(tert-butoxycarbonyl)amino]-3-[4-fluoro-2′-hydroxy-5-methyl-6′-(trifluoromethyl)-[1,1′-biphenyl]-3-yl]propanoate (350 mg, 32.5%) as a yellow oil. LC-MS: (ES+H, m/z) [M+H]⁺=486.2.

### Step 2: Preparation of ethyl (3S)-3-amino-3-[4-fluoro-2′-hydroxy-5-methyl-6′-(trifluoromethyl)-[1,1′-biphenyl]-3-yl]propanoate hydrochloride

To a stirred solution of ethyl (3S)-3-[(tert-butoxycarbonyl)amino]-3-[4-fluoro-2′-hydroxy-5-methyl-6′-(trifluoromethyl)-[1,1′-biphenyl]-3-yl]propanoate (500 mg, 1.03 mmol, 1.00 equiv.) in CH₂Cl₂ (6 mL) was added HCl (gas) in 1,4-dioxane (3 mL, 4 M) dropwise at 0° C. under nitrogen atmosphere. The resulting mixture was stirred for 3 h at room temperature and then concentrated to provide ethyl (3S)-3-amino-3-[4-fluoro-2′-hydroxy-5-methyl-6′-(trifluoromethyl)-[1,1′-biphenyl]-3-yl]propanoate hydrochloride (470 mg, crude) as a yellow oil. LC-MS: (ES+H, m/z) [M+H]⁺=386.1

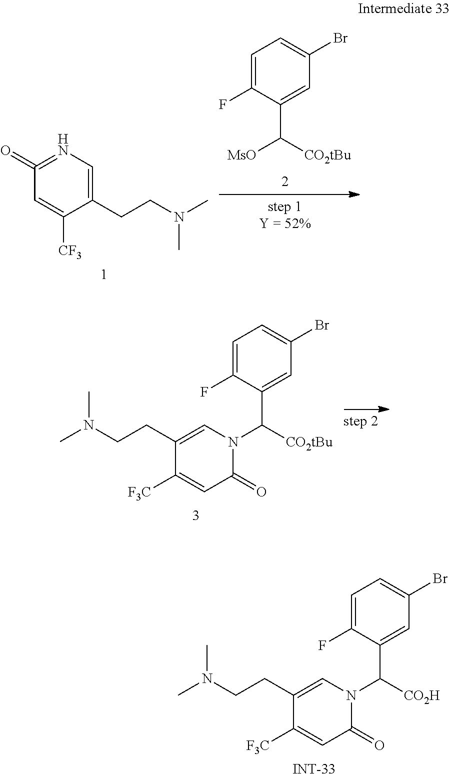

### Step 1: Preparation of tert-butyl 2-(5-bromo-2-fluorophenyl)-2-{5-[2-(dimethylamino)ethyl]-2-oxo-4-(trifluoromethyl)pyridin-1-yl}acetate

To a stirred solution of tert-butyl 2-(5-bromo-2-fluorophenyl)-2-(methanesulfonyloxy)acetate (654 mg, 1.70 mmol, 1.00 equiv.) and 5-[2-(dimethylamino)ethyl]-4-(trifluoromethyl)-1H-pyridin-2-one (400 mg, 1.70 mmol, 1.00 equiv.) in CH₃CN (12 mL) was added K₂CO₃ (472 mg, 3.41 mmol, 2.00 equiv.) in portions at room temperature under nitrogen atmosphere. The resulting mixture was stirred for 2 h at 60° C. under nitrogen atmosphere. The mixture was allowed to cool down to room temperature and then diluted with water (50 mL). The resulting mixture was extracted with CH₂Cl₂ (4×100 mL). The combined organic layers were dried over anhydrous Na₂SO₄, filtered, and concentrated. The residue was purified by reversed-phase flash chromatography (C18; mobile phase, MeCN in water (0.1% NH₃·H₂O), 50% to 70% gradient in 15 min; detector, UV 220 nm) to afford tert-butyl 2-(5-bromo-2-fluorophenyl)-2-{5-[2-(dimethylamino)ethyl]-2-oxo-4-(trifluoromethyl)pyridin-1-yl}acetate (463 mg, 52.0%) as a light yellow liquid. LC-MS: (ES+H, m/z) [M+H]⁺=521.05. ¹H NMR (400 MHz, DMSO-d₆) δ 7.77-7.69 (m, 2H), 7.62 (dd, J=6.5, 2.5 Hz, 1H), 7.36 (dd, J=10.0, 8.8 Hz, 1H), 6.88 (s, 1H), 6.53 (s, 1H), 2.56-2.52 (m, 2H), 2.40-2.25 (m, 2H), 2.05 (s, 6H), 1.42 (s, 9H). ¹⁹F NMR (400 MHz, DMSO-d₆) −62.69, −116.59.

### Step 2: Preparation of (5-bromo-2-fluorophenyl)({5-[2-(dimethylamino)ethyl]-2-oxo-4-(trifluoromethyl)pyridin-1-yl})acetic acid

To a stirred solution of tert-butyl 2-(5-bromo-2-fluorophenyl)-2-{5-[2-(dimethylamino)ethyl]-2-oxo-4-(trifluoromethyl)pyridin-1-yl}acetate (400 mg, 0.76 mmol, 1.00 equiv.) in CH₂Cl₂ (4 mL) was added CF₃COOH (4 mL) dropwise at 0° C. under nitrogen atmosphere. The resulting mixture was stirred overnight at 30° C. under nitrogen atmosphere and then cooled to room temperature, and concentrated. The residue was purified by trituration with Et₂O (20 mL) to afford (5-bromo-2-fluorophenyl)({5-[2-(dimethylamino)ethyl]-2-oxo-4-(trifluoromethyl)pyridin-1-yl})acetic acid (485 mg, crude) as a white solid. The crude product was used in the next step directly without further purification. LC-MS: (ES+H, m/z) [M+H]⁺=465.0. ¹H NMR (400 MHz, DMSO-d₆) δ 9.82 (s, 1H), 7.83 (s, 1H), 7.74-7.65 (m, 2H), 7.36-7.27 (m, 1H), 6.95 (s, 1H), 6.54 (s, 1H), 3.15 (t, J=8.5 Hz, 2H), 2.89-2.77 (m, 8H). ¹⁹F NMR (400 MHz, DMSO-d₆) −62.63, −116.49.

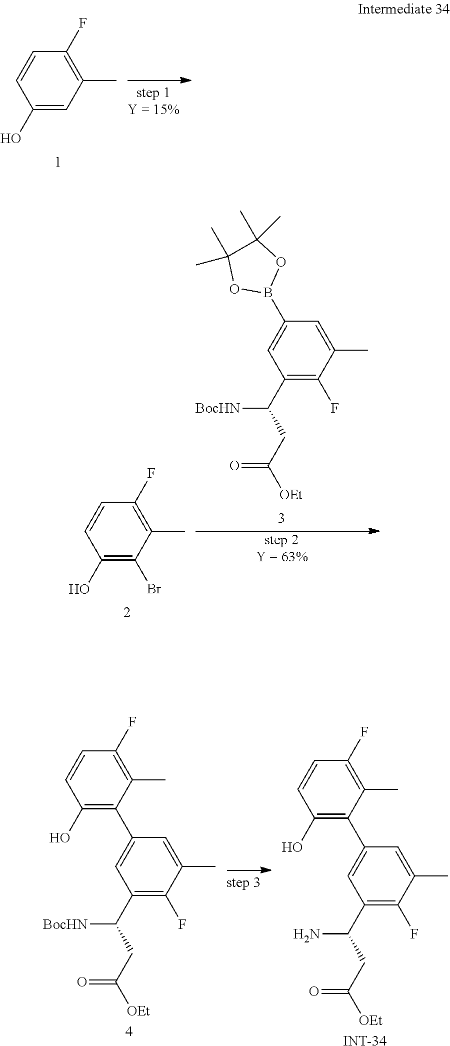

### Step 1: Preparation of 2-bromo-4-fluoro-3-methylphenol

To a stirred mixture of 4-fluoro-3-methylphenol (5.00 g, 39.64 mmol, 1.00 equiv.) in CH₃COOH (150 mL) was added Br₂ (2 mL, 39.64 mmol, 1.00 equiv.) in H₂O (20 mL) dropwise at 0° C. under nitrogen atmosphere. The resulting mixture was stirred for 2 h and then diluted with diethyl ether (500 mL). The residue was washed with Na₂S₂O₃ (1×400 mL), NaHCO₃ (1×400 mL), and then concentrated. The residue was purified by silica gel column chromatography (CH₂Cl₂/PE (0% to 10% gradient in 30 min)) to afford 2-bromo-4-fluoro-3-methylphenol (5 g, crude) as a yellow solid. The crude product (2.5 g) was further purified by Prep-SFC (Column: NB_CHIRALPAK AD, 3*25 cm, 5 μm; Mobile Phase A: CO₂, Mobile Phase B: CH₃OH:EtOH:Hexane=1:1:2 (20 mM NNH₃); Flow rate: 100 mL/min; Gradient: isocratic 10% B; Column Temperature (° C.) 35; Back Pressure (bar) 120; wavelength: 285/260 nm; Sample Solvent: CH3OH-HPLC; Injection Volume: 0.2 mL) to afford 2-bromo-4-fluoro-3-methylphenol (1.2 g, 14.7%) as a yellow solid. ¹H NMR (400 MHz, CDCl₃) δ 6.93 (t, J=8.8 Hz, 1H), 6.85 (dd, J=9.0, 4.9 Hz, 1H), 2.33 (d, J=2.4 Hz, 3H).

### Step 2: Preparation of ethyl (3S)-3-[(tert-butoxycarbonyl)amino]-3-{3′,4-difluoro-6′-hydroxy-2′,5-dimethyl-[1,1′-biphenyl]-3-yl}propanoate

To a stirred mixture of 2-bromo-4-fluoro-3-methylphenol (190 mg, 0.93 mmol, 1.05 equiv.) and ethyl (3S)-3-[(tert-butoxycarbonyl)amino]-3-[2-fluoro-3-methyl-5-(4,4,5,5-tetramethyl-1,3,2-dioxaborolan-2-yl)phenyl]propanoate (400 mg, 0.88 mmol, 1.00 equiv.) in dioxane (10 mL) and H₂O (1 mL) were added K₂CO₃ (367 mg, 2.65 mmol, 3.00 equiv.) and Pd(dppf)Cl₂ (64 mg, 0.08 mmol, 0.10 equiv.) at room temperature under nitrogen atmosphere. The reaction mixture was stirred for 2 h at 100° C. and then cooled to room temperature. The resulting mixture was filtered, and the filter cake was washed with EtOAc (3×100 mL). The combined filtrates were concentrated under reduced pressure. The residue was purified by silica gel column chromatography (PE/EtOAc (0% to 30% gradient in 30 min)) to afford ethyl (3S)-3-[(tert-butoxycarbonyl)amino]-3-{3′,4-difluoro-6′-hydroxy-2′,5-dimethyl-[1,1′-biphenyl]-3-yl}propanoate (250 mg, 62.7%) as a white solid. LC-MS: (ES+H, m/z) [M+H]⁺=450.2.

### Step 3: Preparation of ethyl (3S)-3-amino-3-{3′,4-difluoro-6′-hydroxy-2′,5-dimethyl-[1,1′-biphenyl]-3-yl}propanoate

To a stirred mixture of ethyl (3S)-3-[(tert-butoxycarbonyl)amino]-3-{3′,4-difluoro-6′-hydroxy-2′,5-dimethyl-[1,1′-biphenyl]-3-yl}propanoate (250 mg, 0.55 mmol, 1.00 equiv.) in CH₂Cl₂ (2 mL) was added HCl (gas) in 1,4-dioxane (4 mL, 4 M) at room temperature under nitrogen atmosphere. The resulting mixture was stirred for 1 h at room temperature and then concentrated. The residue was purified by reversed-phase flash chromatography (C18 gel; mobile phase, MeCN in water (0.1% NH₃·H₂O), 10% to 60% gradient in 30 min; detector, UV 220 nm) to afford ethyl (3S)-3-amino-3-{3′,4-difluoro-6′-hydroxy-2′,5-dimethyl-[1,1′-biphenyl]-3-yl}propanoate (180 mg, 92.6%) as a white solid. LC-MS: (ES+H, m/z) [M+H]⁺=350.1.

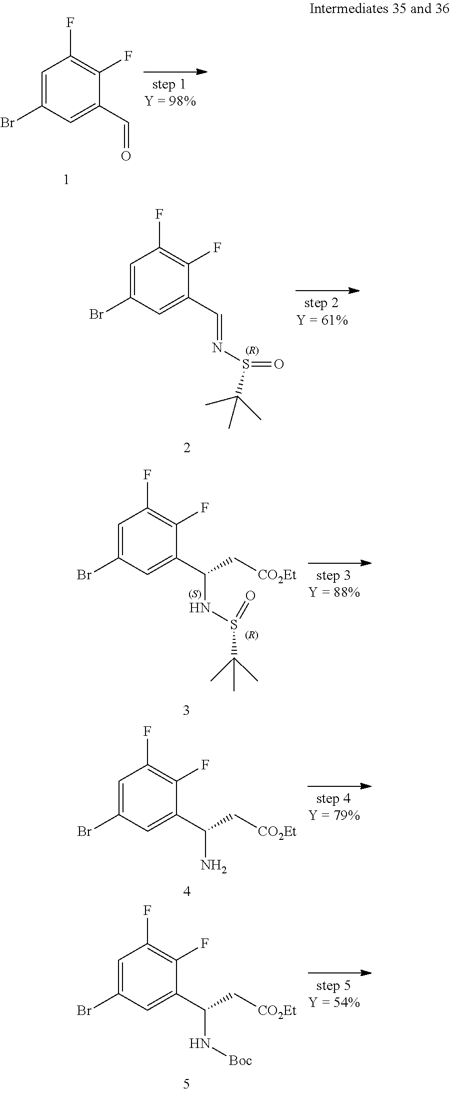

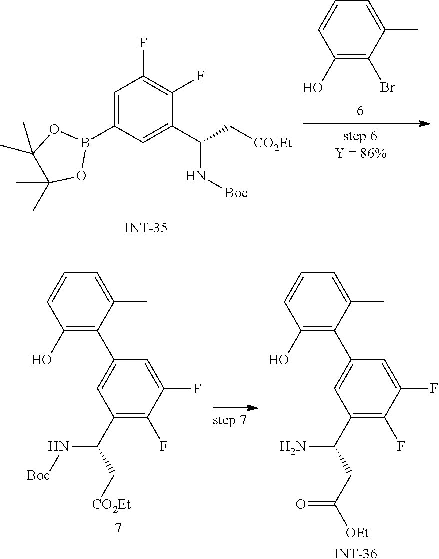

### Step 1: Preparation of (R)—N-[(1Z)-(5-bromo-2,3-difluorophenyl)methylidene]-2-methylpropane-2-sulfinamide

To a stirred solution of 5-bromo-2,3-difluorobenzaldehyde (9.00 g, 40.72 mmol, 1.00 equiv.) and (R)-2-methylpropane-2-sulfinamide (5.43 g, 44.79 mmol, 1.10 equiv.) in THF (90 mL) was added Ti(Oi-Pr)₄ (23.15 g, 81.44 mmol, 2.00 equiv.) dropwise at 20° C. under nitrogen atmosphere. The resulting mixture was stirred for 1 h at 35° C. under nitrogen atmosphere. The mixture was allowed to cool down to 0° C. and quenched with water at 0° C. The resulting mixture was diluted and extracted with ethyl acetate (100 mL). The resulting mixture was extracted with EtOAc (3×100 mL) and the combined organic layers were washed with brine (2×100 mL), dried over anhydrous Na₂SO₄, filtered, and concentrated. The residue was purified by silica gel column chromatography (PE/EA (50:1 to 7:1) to afford (R)—N-[(1Z)-(5-bromo-2,3-difluorophenyl)methylidene]-2-methylpropane-2-sulfinamide (13.00 g, 98.4%) as an off-white solid. LC-MS: (ES+H, m/z) [M+H]⁺=323.8. ¹H NMR (400 MHz, CDCl₃) δ 8.74 (s, 1H), 7.82-7.78 (m, 1H), 7.42-7.38 (m, 1H), 1.21 (s, 9H).

### Step 2: Preparation of ethyl (3S)-3-(5-bromo-2,3-difluorophenyl)-3-{[(R)-2-methylpropane-2-sulfinyl]amino}propanoate

To a stirred mixture of Zn (7.26 g, 111.04 mmol, 4.00 equiv.) and THF (75 mL) was added TMSCl (0.60 g, 5.55 mmol, 0.20 equiv.) dropwise at 20° C. under nitrogen atmosphere. The resulting mixture was stirred for 1 h at 50° C. under nitrogen atmosphere. The mixture was allowed to cool down to 20° C. and ethyl bromoacetate (11.59 g, 69.40 mmol, 2.50 equiv.) was added dropwise at 20° C. under nitrogen atmosphere. The resulting mixture was stirred for an additional 1 h at 60° C. The mixture was then cooled to 0° C. and treated with a solution of (R)—N-[(1Z)-(5-bromo-2,3-difluorophenyl)methylidene]-2-methylpropane-2-sulfinamide (9.00 g, 27.76 mmol, 1.00 equiv.) in THF (15 mL) dropwise. The resulting mixture was stirred for an additional 1 h at room temperature, cooled to 0° C., and quenched with water at 0° C. The resulting mixture was diluted with water (200 mL), and extracted with EtOAc (3×200 mL). The combined organic layers were washed with brine (2×200 mL), dried over anhydrous Na₂SO₄, filtered, and concentrated. The residue was purified by silica gel column chromatography (PE/EA (60:1 to 5:1) to afford ethyl (3S)-3-(5-bromo-2,3-difluorophenyl)-3-{[(R)-2-methylpropane-2-sulfinyl]amino}propanoate (7.00 g, 61.1%) as an off-white oil. LC-MS: (ES+H, m/z) [M+H]⁺=411.85. ¹H NMR (300 MHz, DMSO-d₆) δ 7.73 (ddd, J=9.7, 6.9, 2.4 Hz, 1H), 7.57 (dt, J=5.1, 2.1 Hz, 1H), 5.84 (d, J=7.0 Hz, 1H), 4.95-4.92 (m, 1H), 4.03 (q, J=7.1 Hz, 2H), 3.07-2.84 (m, 2H), 1.12 (t, J=7.1 Hz, 3H), 1.05 (s, 9H).

### Step 3: Preparation of ethyl (S)-3-amino-3-(5-bromo-2,3-difluorophenyl)propanoate

To a stirred mixture of ethyl (S)-3-(5-bromo-2,3-difluorophecan)-3-(((R)-tert-butylsulfinyl)amino)propanoate (2.00 g, 5.07 mmol, 1.00 equiv.) in DCM (5 mL) was added HCl (gas) in 1,4-dioxane (10 mL, 4 M) dropwise at room temperature under nitrogen atmosphere. The resulting mixture was stirred for 2 h at room temperature under nitrogen atmosphere and then concentrated under vacuum to afford ethyl (S)-3-amino-3-(5-bromo-2,3-difluorophenyl)propanoate (1.3 g, 88.3%) as a yellow oil. LC-MS: (ES+H, m/z) [M+H]⁺=308.0.

### Step 4: Preparation of ethyl (3S)-3-(5-bromo-2,3-difluorophenyl)-3-[(tert-butoxycarbonyl)amino]propanoate

To a stirred mixture of ethyl (3S)-3-amino-3-(5-bromo-2,3-difluorophenyl)propanoate (1.47 g, 4.77 mmol, 1.00 equiv.) in CH₂Cl₂ (20 mL) was added Boc₂O (2.08 g, 9.54 mmol, 2.00 equiv.) and DIEA (1.85 g, 14.31 mmol, 3.00 equiv.) at room temperature. After 2 h at room temperature the resulting mixture was diluted with water (50 mL) and extracted with CH₂Cl₂ (3×50 mL). The combined organic layers were washed with brine (3×50 mL), dried over anhydrous Na₂SO₄, filtered, and concentrated. The residue was purified by silica gel column chromatography (PE/EtOAc (0% to 20% gradient in 25 min) to afford ethyl (3S)-3-(5-bromo-2,3-difluorophenyl)-3-[(tert-butoxycarbonyl)amino]propanoate (1.53 g, 78.5%) as a white solid. LC-MS: (ES−H, m/z) [M−H]⁻=406.0.

### Step 5: Preparation of ethyl (3S)-3-[(tert-butoxycarbonyl)amino]-3-[2,3-difluoro-5-(4,4,5,5-tetramethyl-1,3,2-dioxaborolan-2-yl)phenyl]propanoate

To a stirred mixture of ethyl (3S)-3-(5-bromo-2,3-difluorophenyl)-3-[(tert-butoxycarbonyl)amino]propanoate (1 g, 2.45 mmol, 1.00 equiv.) in dioxane (10 mL) was added Pd(dppf)Cl₂·CH₂Cl₂ (0.14 g, 0.24 mmol, 0.10 equiv.), KOAc (0.72 g, 7.35 mmol, 3.00 equiv.) and portion-wise addition of 4,4,5,5-tetramethyl-2-(4,4,5,5-tetramethyl-1,3,2-dioxaborolan-2-yl)-1,3,2-dioxaborolane (0.62 g, 2.45 mmol, 1.00 equiv.) at room temperature. The resulting mixture was stirred for 2 h at 100° C. under nitrogen atmosphere, cooled to room temperature and concentrated. The residue was purified by silica gel column chromatography (PE/EtOAc (0% to 20% gradient in 25 min) to afford ethyl (3S)-3-[(tert-butoxycarbonyl)amino]-3-[2,3-difluoro-5-(4,4,5,5-tetramethyl-1,3,2-dioxaborolan-2-yl)phenyl]propanoate (610 mg, 54.7%) as a yellow oil. LC-MS: (ES+H, m/z) [M+H−Boc]⁺=356.1.

### Step 6: Preparation of ethyl (3S)-3-[(tert-butoxycarbonyl)amino]-3-{4,5-difluoro-2′-hydroxy-6′-methyl-[1,1′-biphenyl]-3-yl}propanoate

To a stirred mixture of ethyl (3S)-3-[(tert-butoxycarbonyl)amino]-3-[2,3-difluoro-5-(4,4,5,5-tetramethyl-1,3,2-dioxaborolan-2-yl)phenyl]propanoate (500 mg, 1.09 mmol, 1.00 equiv.) and 2-bromo-3-methylphenol (246 mg, 1.32 mmol, 1.20 equiv.) in dioxane (10 mL) and H₂O (1 mL) were added K₂CO₃ (455 mg, 3.29 mmol, 3.00 equiv.) and Pd(dppf)Cl₂·CH₂Cl₂ (89 mg, 0.11 mmol, 0.10 equiv.) at room temperature under nitrogen atmosphere. The resulting mixture was stirred for 2 h at 100° C. under nitrogen atmosphere and then cooled to room temperature and concentrated. The residue was purified by silica gel column chromatography (PE/EtOAc (0% to 20% gradient in 25 min) to afford ethyl (3S)-3-[(tert-butoxycarbonyl)amino]-3-{4,5-difluoro-2′-hydroxy-6′-methyl-[1,1′-biphenyl]-3-yl}propanoate (410 mg, 85.7%) as a light yellow oil. LC-MS: (ES+H, m/z) [M+H−Boc]⁺=336.1.

### Step 7: Preparation of ethyl (3S)-3-amino-3-{4,5-difluoro-2′-hydroxy-6′-methyl-[1,1′-biphenyl]-3-yl}propanoate hydrochloride

To a stirred mixture of ethyl (3S)-3-[(tert-butoxycarbonyl)amino]-3-{4,5-difluoro-2′-hydroxy-6′-methyl-[1,1′-biphenyl]-3-yl}propanoate (410 mg, 0.94 mmol, 1.00 equiv.) in CH₂Cl₂ (3 mL) was added HCl (gas) in 1,4-dioxane (3 mL, 4 M) at room temperature under nitrogen atmosphere. The resulting mixture was stirred for 1 h at room temperature under nitrogen atmosphere and then concentrated to provide ethyl (3S)-3-amino-3-{4,5-difluoro-2′-hydroxy-6′-methyl-[1,1′-biphenyl]-3-yl}propanoate hydrochloride (370 mg, crude) as a colorless oil. LC-MS: (ES+H, m/z) [M+H]⁺=336.1.

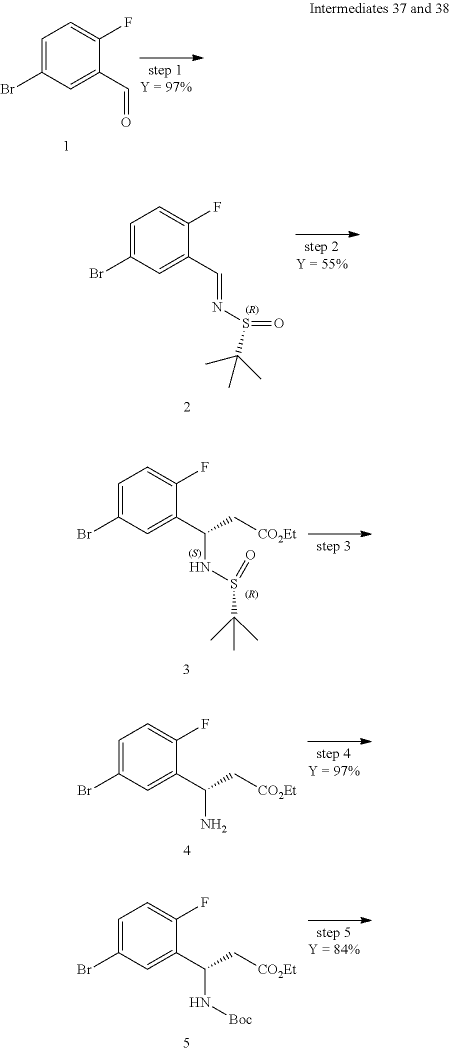

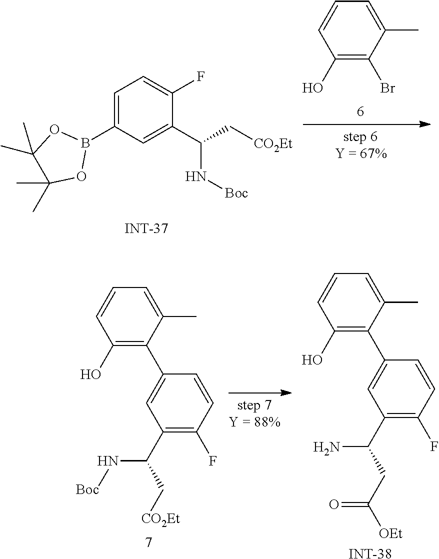

### Step 1: Preparation of (R)—N-[(1E)-(5-bromo-2-fluorophenyl)methylidene]-2-methylpropane-2-sulfinamide

To a stirred solution of 5-bromo-2-fluorobenzaldehyde (10.50 g, 51.72 mmol, 1.00 eq can) and (R)-2-methylpropane-2-sulfinamide (7.52 g, 62.06 mmol, 1.20 equiv.) in THF (150 mL) was added tetrakis(propan-2-yloxy) titanium (22.05 g, 77.58 mmol, 1.50 equiv.) dropwise at room temperature under nitrogen atmosphere. The resulting mixture was stirred for 2 h at 50° C. under nitrogen atmosphere and then cooled to room temperature. To the above mixture was added water (500 mL) and EtOAc (200 mL). The resulting mixture was stirred for an additional 10 min at room temperature. The resulting mixture was filtered and the filter cake was washed with EtOAc (3×100 mL). The aqueous layer was extracted with EtOAc (2×500 mL) and the combined organic layers were washed with brine (3×500 mL), dried over anhydrous Na₂SO₄, filtered, and concentrated to provide (R)—N-[(1E)-(5-bromo-2-fluorophenyl)methylidene]-2-methylpropane-2-sulfinamide (15.40 g, 97.2%) as a light yellow oil. LC-MS: (ES+H, m/z) [M+H]⁺=305.9.

### Step 2: Preparation of ethyl (3S)-3-(5-bromo-2-fluorophecan)-3-{[(R)-2-methylpropane-2-sulfinyl] amino} propanoate

A solution of Zn (12.81 g, 195.95 mmol, 4.00 equiv.) in THF (200 mL) was treated with TMSCl (1.06 g, 9.79 mmol, 0.20 equiv.) for 1 h at 60° C. under nitrogen atmosphere followed by the addition of ethyl 2-bromoacetate (20.45 g, 122.47 mmol, 2.50 equiv.) dropwise at 0° C. The resulting mixture was stirred for 1 h at 60° C. under nitrogen atmosphere. To the above mixture was added (R)—N-[(1E)-(5-bromo-2-fluorophenyl) methylidene]-2-methylpropane-2-sulfinamide (15.00 g, 48.98 mmol, 1.00 equiv.) dropwise at 0° C. The resulting mixture was stirred for 2 h at room temperature and then quenched with water at 0° C. The resulting mixture was filtered and the filter cake was washed with EtOAc (3×200 mL). The combined filtrate was concentrated under reduced pressure and the residue was purified by silica gel column chromatography (PE/EtOAc (0% to 30% in 20 min) to afford ethyl (3S)-3-(5-bromo-2-fluorophecan)-3-1{[(R)-2-methylpropane-2-sulfinyl] amino}propanoate (10.75 g, 55.6%) as a colorless oil. LC-MS: (ES+H, m/z) [M+H]⁺=393.90. ¹H NMR (300 MHz, CDCl₃) δ 7.55-7.36 (m, 2H), 7.01-6.91 (m, 1H), 5.03 (q, J=6.1 Hz, 1H), 4.75 (d, J=5.9 Hz, 1H), 4.14 (q, J=7.2 Hz, 2H), 3.02-2.86 (m, 2H), 1.25 (d, J=6.5 Hz, 12H). ¹⁹F NMR (282 MHz, CDCl₃) δ −119.47.

### Step 3: Preparation of ethyl (3S)-3-amino-3-(5-bromo-2-fluorophenyl)propanoate

A solution of ethyl (3S)-3-(5-bromo-2-fluorophecan)-3-{[(R)-2-methylpropane-2-sulfinyl]amino}propanoate (6.30 g, 15.98 mmol, 100 equiv.) and HCl (gas) in 1,4-dioxane (4 M, 100 mL) in CH₂Cl₂ (50 mL) was stirred overnight at room temperature under nitrogen atmosphere and then concentrated under reduced pressure. The residue was dissolved in water (150 mL) and washed with EtOAc (2×50 mL). The aqueous layer was then basified to pH 10 with NH₃·H₂O and extracted with EtOAc (3×60 mL). The combined organic layers were washed with brine (3×50 mL), dried over anhydrous Na₂SO₄, filtered, and concentrated to provide ethyl (3S)-3-amino-3-(5-bromo-2-fluorophenyl)propanoate (4.30 g, crude, ee %=98.5%) as a light yellow oil. The crude product was used in the next step directly without further purification. LC-MS: (ES+H, m/z) [M+H]⁺=290.05. ¹H NMR (300 MHz, CDCl₃) δ 7.62 (dd, J=6.5, 2.5 Hz, 1H), 7.39-7.32 (m, 1H), 6.94 (dd, J=10.1, 8.7 Hz, 1H), 4.65 (dd, J=8.6, 4.7 Hz, 1H), 4.17 (q, J=7.1 Hz, 2H), 2.79-2.59 (m, 2H), 1.85 (s, 2H), 1.26 (t, J=7.1 Hz, 3H). ¹⁹F NMR (282 MHz, CDCl₃) δ −120.93.

### Step 4: Preparation of ethyl (3S)-3-(5-bromo-2-fluorophenyl)-3-[(tert-butoxycarbonyl) amino]propanoate

A solution of ethyl (3S)-3-amino-3-(5-bromo-2-fluorophenyl)propanoate (4.20 g, 14.48 mmol, 1.00 equiv.), (Boc)₂O (6.32 g, 28.95 mmol, 2.00 equiv.) and DIEA (5.61 g, 43.43 mmol, 3.00 equiv.) in DCM (150 mL) was stirred overnight at room temperature under nitrogen atmosphere. The resulting mixture was diluted with water (150 mL) and extracted with CH₂Cl₂ (3×100 mL). The combined organic layers were washed with brine (3×100 mL), dried over anhydrous Na₂SO₄, filtered, and concentrated. The residue was purified by silica gel column chromatography (PE/EtOAc (0% to 50% in 20 min) to afford ethyl (3S)-3-(5-bromo-2-fluorophenyl)-3-[(tert-butoxycarbonyl)amino]propanoate (5.50 g, 97.4%) as a white solid. LC-MS: (ES+H, m/z) [M+H−tBu]⁺=333.95. ¹H NMR (300 MHz, CDCl₃) δ 7.45 (dd, J=6.7, 2.5 Hz, 1H), 7.39-7.32 (m, 1H), 6.93 (dd, J=10.1, 8.7 Hz, 1H), 5.66 (s, 1H), 4.08 (q, J=7.1 Hz, 2H), 2.82 (d, J=6.0 Hz, 2H), 1.43 (s, 9H), 1.18 (t, J=7.1 Hz, 3H). ¹⁹F NMR (282 MHz, CDCl₃) δ −120.24.

### Step 5: Preparation of ethyl (3S)-3-[(tert-butoxycarbonyl)amino]-3-[2-fluoro-5-(4,4,5,5-tetramethyl-1,3,2-dioxaborolan-2-yl)phenyl]propanoate

A mixture of ethyl (3S)-3-(5-bromo-2-fluorophenyl)-3-[(tert-butoxycarbonyl)amino]propanoate (5.40 g, 13.84 mmol, 1.00 equiv.), bis(pinacolato)diboron (7.03 g, 27.67 mmol, 2.00 equiv.), Pd(dppf)Cl₂ (1.01 g, 1.38 mmol, 0.10 equiv.) and KOAc (2.72 g, 27.67 mmol, 2.00 equiv.) in 1,4-dioxane (100 mL) was stirred for 2 h at 100° C. under nitrogen atmosphere. The mixture was cooled to room temperature, diluted with water (100 mL), and extracted with EtOAc (3×150 mL). The combined organic layers were washed with brine (3×150 mL), dried over anhydrous Na₂SO₄, filtered, and concentrated. The residue was purified by silica gel column chromatography (petroleum ether/EtOAc (0% to 60% in 20 min) to afford ethyl (3S)-3-[(tert-butoxycarbonyl)amino]-3-[2-fluoro-5-(4,4,5,5-tetramethyl-1,3,2-dioxaborolan-2-yl)phenyl]propanoate (5.10 g, 84.3%) as a light brown oil. LC-MS: (ES+H, m/z) [M+H−tBu]⁺=382.10. ¹H NMR (300 MHz, CDCl₃) δ 7.80-7.67 (m, 2H), 7.04 (dd, J=11.0, 8.2 Hz, 1H), 5.48 (s, 1H), 5.35 (s, 1H), 4.10 (p, J=7.1 Hz, 2H), 2.94-2.72 (m, 2H), 1.43 (s, 9H), 1.34 (s, 12H), 1.19 (t, J=7.1 Hz, 3H). ¹⁹F NMR (282 MHz, CDCl₃) δ −113.67.

### Step 6: Preparation of ethyl (3S)-3-[(tert-butoxycarbonyl)amino]-3-{4-fluoro-2′-hydroxy-6′-methyl-[1,1′-biphenyl]-3-yl}propanoate

A mixture of ethyl (3S)-3-[(tert-butoxycarbonyl)amino]-3-[2-fluoro-5-(4,4,5,5-tetramethyl-1,3,2-dioxaborolan-2-yl)phenyl]propanoate (5.00 g, 11.43 mmol, 1.00 equiv.), 2-bromo-3-methylphenol (2.25 g, 12.00 mmol, 1.05 equiv.), K₂CO₃ (3.16 g, 22.87 mmol, 2.00 equiv.) and Pd(dppf)Cl₂ (418 mg, 0.57 mmol, 0.05 equiv.) in 1,4-dioxane (100 mL) and H₂O (5 mL) was stirred for 2 h at 100° C. under nitrogen atmosphere. The mixture was allowed to cool down to room temperature, diluted with water (100 mL) and extracted with EtOAc (3×150 mL). The combined organic layers were washed with brine (3×150 mL), dried over anhydrous Na₂SO₄, filtered, and concentrated. The residue was purified by silica gel column chromatography (petroleum ether/EtOAc (0% to 60% in 20 min) to afford ethyl (3S)-3-[(tert-butoxycarbonyl)amino]-3-{4-fluoro-2′-hydroxy-6′-methyl-[1,1′-biphenyl]-3-yl}propanoate (3.20 g, 67.0%) as a light yellow oil. LC-MS: (ES+H, m/z) [M+Na+]⁺=440.2. ¹H NMR (300 MHz, CDCl₃) δ 7.31 (d, J=7.1 Hz, 1H), 7.23-7.15 (m, 3H), 6.86 (d, J=7.8 Hz, 2H), 5.77 (d, J=8.7 Hz, 1H), 5.38 (s, 1H), 4.80 (s, 1H), 4.11 (ddd, J=11.7, 8.4, 6.0 Hz, 2H), 2.91 (d, J=5.7 Hz, 2H), 2.06 (s, 3H), 1.43 (s, 9H), 1.20 (t, J=7.1 Hz, 3H). ¹⁹F NMR (282 MHz, CDCl₃) δ −119.04.

### Step 7: Preparation of ethyl (3S)-3-amino-3-{4-fluoro-2′-hydroxy-6′-methyl-[1,1′-biphenyl]-3-yl}propanoate

A solution of ethyl (3S)-3-[(tert-butoxycarbonyl)amino]-3-{4-fluoro-2′-hydroxy-6′-methyl-[1,1′-biphenyl]-3-yl}propanoate (3.00 g, 7.19 mmol, 1.00 equiv.) and HCl gas in 1,4-dioxane (4 M, 50 mL) in CH₂Cl₂ (50 mL) was stirred for 4 h at room temperature under nitrogen atmosphere and then concentrated under reduced pressure. The residue was purified by reversed-phase flash chromatography (C18; mobile phase, MeCN in water (0.1% NH₃·H₂O), 40% to 70% gradient in 15 min; detector, UV 220 nm) to provide ethyl (3S)-3-amino-3-{4-fluoro-2′-hydroxy-6′-methyl-[1,1′-biphenyl]-3-yl}propanoate (2.00 g, 87.7%) as a brown solid. LC-MS: (ES+H, m/z) [M+H]⁺=318.05.

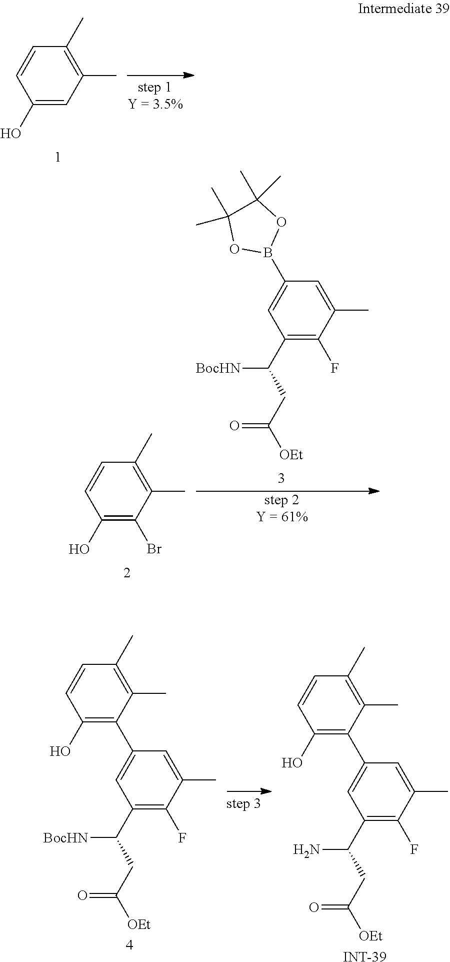

### Step 1: Preparation of 2-bromo-3,4-dimethylphenol

To a stirred mixture of 3,4-dimethylphenol (5.00 g, 40.93 mmol, 1.00 equiv.) in CH₃COOH (120 mL) was added Br₂ (2.1 mL, 40.93 mmol, 1.00 equiv.) in H₂O (17 mL) and CH₃COOH (10 mL) dropwise at 0° C. under nitrogen atmosphere. The resulting mixture was stirred for 2 h at room temperature and then concentrated under reduced pressure. The resulting mixture was diluted with diethyl ether (600 mL) and washed with Na₂S₂O₃ (aq. 0.63 M) (1×500 mL), and sat. NaHCO₃ (aq.) (1×500 mL). The organic layer was then dried over anhydrous Na₂SO₄, filtered, and concentrated. The residue was purified first by silica gel column chromatography (PE/CH₂Cl₂ (0%-30% in 50 min) to afford 2-bromo-3,4-dimethylphenol (700 mg, crude) as a white solid and then further purified by Prep-SFC (Column: CHIRALPAK IG-3, 4.6*50 mm, 3 μm; Mobile Phase A: CO₂, Mobile Phase B: MeOH-EtOH-HEX-1-1-2; Flow rate: 100 mL/min; Gradient: isocratic 10% B; Column Temperature (° C.) 35; Back Pressure (bar) 100; wavelength: 284/220 nm; RT1 (min) 7.65; RT2 (min) 8.57; Sample Solvent: CH₃OH:CH₂Cl₂=16:1; Injection Volume: 0.2 mL) to afford 2-bromo-3,4-dimethylphenol (290 mg, 3.5%) as a light yellow solid. LC-MS: (ES−H, m/z) [M−H]⁻=199.98. ¹H NMR (300 MHz, DMSO-d₆) δ 9.79 (s, 1H), 6.94 (d, J=8.2 Hz, 1H), 6.69 (d, J=8.1 Hz, 1H), 2.27 (s, 3H), 2.19 (s, 3H).

### Step 2: Preparation of ethyl (3S)-3-[(tert-butoxycarbonyl)amino]-3-{4-fluoro-6′-hydroxy-2′,3′,5-trimethyl-[1,1′-biphenyl]-3-yl}propanoate

To a stirred mixture of 2-bromo-3,4-dimethylphenol (220 mg, 1.09 mmol, 1.00 equiv.) and ethyl (3S)-3-[(tert-butoxycarbonyl)amino]-3-[2-fluoro-3-methyl-5-(4,4,5,5-tetramethyl-1,3,2-dioxaborolan-2-yl)phenyl]propanoate (494 mg, 1.09 mmol, 1.00 equiv.) in 1,4-dioxane (5 mL):H₂O (0.5 mL) were added K₂CO₃ (454 mg, 3.28 mmol, 3.00 equiv.) and Pd(dppf)Cl₂·CH₂Cl₂ (178 mg, 0.22 mmol, 0.20 equiv.) at room temperature. The resulting mixture was stirred for 2 h at 100° C. under nitrogen atmosphere and then cooled to room temperature and concentrated. The residue was purified by silica gel column chromatography (PE/EtOAc (20% to 100% in 40 min) to afford ethyl (3S)-3-[(tert-butoxycarbonyl)amino]-3-{4-fluoro-6′-hydroxy-2′,3′,5-trimethyl-[1,1′-biphenyl]-3-yl}propanoate (300 mg, 61.5%) as a light yellow solid. LC-MS: (ES+H, m/z) [M+H]⁺=446.2. ¹H NMR (300 MHz, DMSO-d₆) δ 8.82 (s, 1H), 7.48 (d, J=8.4 Hz, 1H), 7.03 (d, J=6.8 Hz, 1H), 6.93 (dd, J=8.2, 3.5 Hz, 2H), 6.65 (d, J=8.1 Hz, 1H), 5.39-5.17 (m, 1H), 4.08-4.00 (m, 2H), 2.66 (d, J=7.5 Hz, 2H), 2.24 (d, J=1.9 Hz, 3H), 2.15 (s, 3H), 1.83 (d, J=6.8 Hz, 3H), 1.34 (s, 9H), 1.19-1.11 (m, 3H).

### Step 3: Preparation of ethyl (3S)-3-amino-3-{4-fluoro-6′-hydroxy-2′,3′,5-trimethyl-[1,1′-biphenyl]-3-yl}propanoate hydrochloride

To a stirred solution of ethyl (3S)-3-[(tert-butoxycarbonyl)amino]-3-{4-fluoro-6′-hydroxy-2′,3′,5-trimethyl-[1,1′-biphenyl]-3-yl}propanoate (250 mg, 0.56 mmol, 1.00 equiv.) in CH₂Cl₂ (2 mL) was added HCl (gas) in 1,4-dioxane (2 mL, 8.00 mmol, 14.26 equiv., 4 M) dropwise at room temperature under nitrogen atmosphere. The resulting mixture was stirred for 1 h at room temperature and then concentrated. The resulting solid was treated with THF (10 mL) and concentrated to provide ethyl (3S)-3-amino-3-{4-fluoro-6′-hydroxy-2′,3′,5-trimethyl-[1,1′-biphenyl]-3-yl}propanoate hydrochloride (240 mg, crude) as a light yellow solid. LC-MS: (ES+H, m/z) [M+H]⁺=346.2.

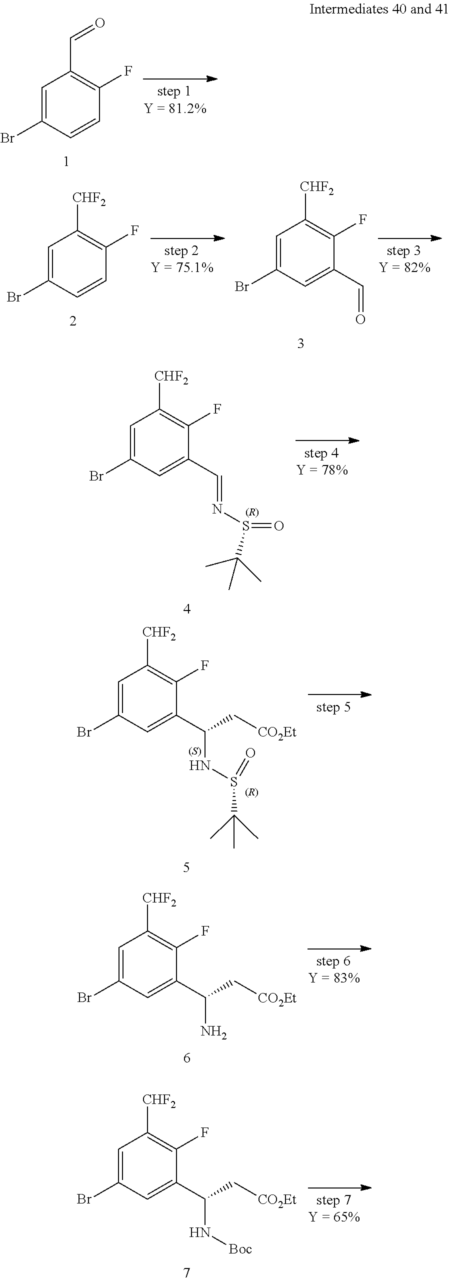

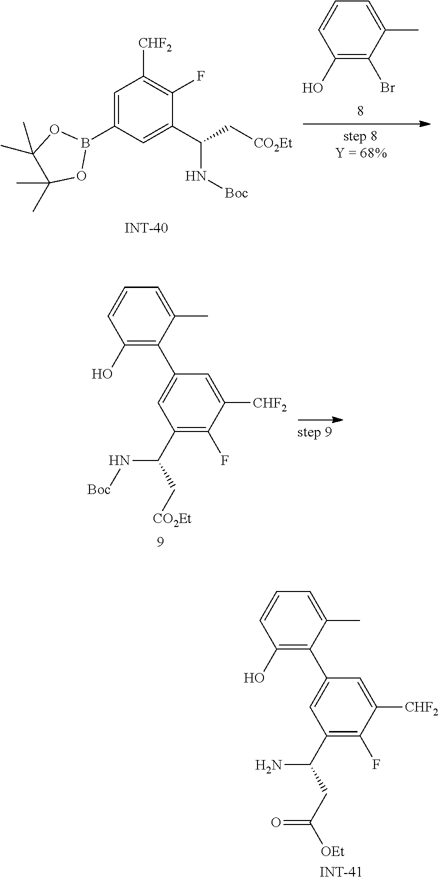

### Step 1: Preparation of 4-bromo-2-(difluoromethyl)-1-fluorobenzene

To a stirred mixture of 5-bromo-2-fluorobenzaldehyde (50.00 g, 246.29 mmol, 1.00 equiv.) in CH₂Cl₂ (500 mL) was added BAST (108.98 g, 492.59 mmol, 2.00 equiv.) dropwise at 0° C. under nitrogen atmosphere. The resulting mixture was stirred overnight at room temperature and then quenched with water at 0° C. The resulting mixture was diluted with water (500 mL and then extracted with CH₂Cl₂ (3×500 mL). The combined organic layers were washed with brine (3×500 mL), dried over anhydrous Na₂SO₄, filtered, and concentrated. The residue was purified by silica gel column chromatography (PE/CH₂Cl₂ (0 to 5% gradient in 30 min)) to afford 4-bromo-2-(difluoromethyl)-1-fluorobenzene (45.00 g, 81.2%) as a yellow solid. ¹H NMR (400 MHz, DMSO-d₆) δ 7.87-7.79 (m, 2H), 7.43-7.35 (m, 1H), 7.20 (t, J=53.9 Hz, 1H). ¹⁹F NMR (377 MHz, DMSO-d₆) δ −113.77, −119.77.

### Step 2: Preparation of 5-bromo-3-(difluoromethyl)-2-fluorobenzaldehyde

To a stirred solution of 4-bromo-2-(difluoromethyl)-1-fluorobenzene (45.00 g, 199.99 mmol, 1.00 equiv.) in THF (400 mL) was added LDA (150 mL, 299.99 mmol, 1.50 equiv., 2 M in THF) dropwise at −78° C. under nitrogen atmosphere. The resulting mixture was stirred for 1 h at −78° C. under nitrogen atmosphere. To the above mixture was added DMF (77 mL, 999.97 mmol, 5.00 equiv.) dropwise at −78° C. The resulting mixture was stirred for 1 h at −78° C. and then quenched with sat. NH₄Cl (aq.) at 0° C. The resulting mixture was diluted with EtOAc (1000 mL), washed with water (3×500 mL), dried over anhydrous Na₂SO₄, filtered, and concentrated. The residue was purified by silica gel column chromatography (PE/EtOAc (0 to 10% gradient in 40 min) to afford 5-bromo-3-(difluoromethyl)-2-fluorobenzaldehyde (38.00 g, 75.1%) as a yellow solid. ¹H NMR (300 MHz, DMSO-d₆) δ 10.15 (s, 1H), 8.19-8.10 (m, 2H), 7.28 (t, J=53.6 Hz, 1H). ¹⁹F NMR (282 MHz, DMSO-d₆) δ −114.11, −126.83.

### Step 3: Preparation of (R,E)-N-(5-bromo-3-(difluoromethyl)-2-fluorobenzylidene)-2-methylpropane-2-sulfinamide

To a stirred mixture of 5-bromo-3-(difluoromethyl)-2-fluorobenzaldehyde (38.00 g, 150.19 mmol, 1.00 equiv.) and (R)-2-methylpropane-2-sulfinamide (20.02 g, 165.21 mmol, 1.10 equiv.) in THF (360 mL) were added tetrakis(propan-2-yloxy)titanium (85.37 g, 300.37 mmol, 2.00 equiv.) dropwise at room temperature under nitrogen atmosphere. The resulting mixture was stirred for 2 h at room temperature and then diluted with EtOAc (300 mL) and water (300 mL) and filtered. The filter cake was washed with EtOAc (3×100 mL), and the combined filtrates were concentrated under reduced pressure. The combined organic layers were washed with brine (3×300 mL), dried over anhydrous Na₂SO₄, filtered, and concentrated. The residue was purified by silica gel column chromatography (PE/EtOAc (0 to 20% gradient in 40 min) to afford (R,E)-N-(5-bromo-3-(difluoromethyl)-2-fluorobenzylidene)-2-methylpropane-2-sulfinamide (44.00 g, 82.2%) as a white solid. LC-MS: (ES−H, m/z) [M+H]⁺=356.0. ¹H NMR (400 MHz, DMSO-d₆) δ 8.64 (s, 1H), 8.28 (dd, J=6.0, 2.6 Hz, 1H), 8.05 (dd, J=6.0, 2.7 Hz, 1H), 7.27 (t, J=53.7 Hz, 1H), 1.21 (s, 9H). ¹⁹F NMR (376 MHz, DMSO-d₆) δ −114.18, −124.78.

### Step 4: Preparation of ethyl (S)-3-(5-bromo-3-(difluoromethyl)-2-fluorophenyl)-3-(((R)-tert-butylsulfinyl)amino)propanoate

To a stirred solution of Zn (14.68 g, 224.59 mmol, 4.00 equiv.) in THF (20 mL) was added TMSCl (2.44 g, 22.46 mmol, 0.40 equiv.) dropwise at room temperature under nitrogen atmosphere. The resulting mixture was stirred for 1 h at 60° C. under nitrogen atmosphere. To the above mixture was added ethyl 2-bromoacetate (23.44 g, 140.37 mmol, 2.50 equiv.) dropwise at room temperature. The resulting mixture was stirred for an additional 1 h at 50° C. and then (R,E)-N-(5-bromo-3-(difluoromethyl)-2-fluorobenzylidene)-2-methylpropane-2-sulfinamide (20.00 g, 56.15 mmol, 1.00 equiv.) in THF (10 mL) was added dropwise at −20° C. The resulting mixture was stirred for 1 h at room temperature and then diluted with water (200 mL) and EtOAc (200 mL) and filtered. The filter cake was washed with EtOAc (3×50 mL) and the aqueous layer was extracted with EtOAc (3×200 mL). The combined organic layers were washed with brine (3×200 mL), dried over anhydrous Na₂SO₄, filtered, and concentrated. The residue was purified by silica gel column chromatography (PE/EtOAc (30% to 60% gradient in 40 min) to afford ethyl (3S)-3-[5-bromo-3-(difluoromethyl)-2-fluorophenyl]-3-{[(R)-2-methylpropane-2-sulfinyl]amino}propanoate (19.50 g, 78.1%) as a yellow oil. LC-MS: (ES+H, m/z) [M+H]⁺=444.0. ¹H NMR (400 MHz, CDCl₃) δ 7.68-7.61 (m, 2H), 6.84 (t, J=54.7 Hz, 1H), 5.05 (dt, J=7.0, 5.5 Hz, 1H), 4.80 (d, J=5.8 Hz, 1H), 4.16-4.08 (m, 2H), 2.93 (qd, J=16.2, 6.1 Hz, 2H), 1.24 (s, 9H), 1.21 (t, J=7.1 Hz, 3H).

### Step 5: Preparation of ethyl (S)-3-amino-3-(5-bromo-3-(difluoromethyl)-2-fluorophenyl)propanoate

To a stirred solution of ethyl (3S)-3-[5-bromo-3-(difluoromethyl)-2-fluorophenyl]-3-{[(S)-2-methylpropane-2-sulfinyl]amino}propanoate (19.30 g, 43.44 mmol, 1.00 equiv.) in CH₂Cl₂ (50 mL) was added HCl (gas) in 1,4-dioxane (150 mL, 600.00 mmol, 13.81 equiv., 4 M) dropwise at room temperature under nitrogen atmosphere. The resulting mixture was stirred for 2 h at room temperature and concentrated. The residue was diluted with water (100 mL) and basified to pH 10 with NH₃·H₂O. The resulting mixture was extracted with EtOAc (3×2 00 mL). The combined organic layers were washed with brine (3×200 mL), dried over anhydrous Na₂SO₄, filtered, and concentrated to provide ethyl (S)-3-amino-3-(5-bromo-3-(difluoromethyl)-2-fluorophenyl)propanoate (14.00 g, 93.7%) as a yellow oil. LC-MS: (ES+H, m/z) [M+H]⁺=340.0.

### Step 6: Preparation of ethyl (S)-3-(5-bromo-3-(difluoromethyl)-2-fluorophenyl)-3-((tert-butoxycarbonyl)amino)propanoate

To a stirred solution of ethyl (S)-3-amino-3-(5-bromo-3-(difluoromethyl)-2-fluorophenyl)propanoate (13.70 g, 40.28 mmol, 1.00 equiv.) in CH₂Cl₂ (140 mL) were added DIEA (15.62 g, 120.83 mmol, 3.00 equiv.) and Boc₂O (17.58 g, 80.56 mmol, 2.00 equiv.) dropwise at 0° C. under nitrogen atmosphere. The resulting mixture was stirred for 2 h at room temperature under nitrogen atmosphere an then diluted with CH₂Cl₂ (400 mL), and washed with water (3×200 mL). The organic layer was washed with brine (3×200 mL), dried over anhydrous Na₂SO₄, filtered, and concentrated. The residue was purified by silica gel column chromatography (PE/EtOAc (0 to 20% gradient in 40 min) to afford ethyl (S)-3-(5-bromo-3-(difluoromethyl)-2-fluorophenyl)-3-((tert-butoxycarbonyl)amino)propanoate (14.80 g, 83.4%) as a white solid. LC-MS: (ES−H, m/z) [M−H]⁻=438.0. ¹1H NMR (400 MHz, DMSO-d₆) δ 7.81 (dd, J=6.3, 2.5 Hz, 1H), 7.73 (dd, J=5.8, 2.5 Hz, 1H), 7.66 (d, J=8.5 Hz, 1H), 7.19 (t, J=53.9 Hz, 1H), 5.22 (q, J=8.0 Hz, 1H), 4.03 (tq, J=7.1, 3.3 Hz, 2H), 2.71 (dt, J=15.4, 8.1 Hz, 2H), 1.35 (s, 9H), 1.12 (t, J=7.1 Hz, 3H).

### Step 7: Preparation of ethyl (S)-3-((tert-butoxycarbonyl)amino)-3-(3-(difluoromethyl)-2-fluoro-5-(4,4,5,5-tetramethyl-1,3,2-dioxaborolan-2-yl)phenyl)propanoate

To a stirred mixture of ethyl (S)-3-(5-bromo-3-(difluoromethyl)-2-fluorophenyl)-3-((tert-butoxycarbonyl)amino)propanoate (4.00 g, 9.09 mmol, 1.00 equiv.) and 4,4,5,5-tetramethyl-2-(4,4,5,5-tetramethyl-1,3,2-dioxaborolan-2-yl)-1,3,2-dioxaborolane (2.31 g, 9.09 mmol, 1.00 equiv.) in 1,4-dioxane (50 mL) were added Pd(dppf)Cl₂·CH₂Cl₂ (0.74 g, 0.91 mmol, 0.10 equiv.) and KOAc (2.68 g, 27.26 mmol, 3.00 equiv.). The resulting mixture was stirred for 2 h at 100° C. under nitrogen atmosphere and then cooled to room temperature and concentrated. The residue was purified by silica gel column chromatography (PE/EtOAc (0 to 20% gradient in 30 min)) to afford ethyl (S)-3-((tert-butoxycarbonyl)amino)-3-(3-(difluoromethyl)-2-fluoro-5-(4,4,5,5-tetramethyl-1,3,2-dioxaborolan-2-yl)phenyl)propanoate (2.90 g, 65.5%) as a yellow oil. LC-MS: (ES+H, m/z) [M+H−tBu]⁺=430.1. ¹H NMR (300 MHz, DMSO-d6) δ 7.95 (d, J=7.3 Hz, 1H), 7.76 (dd, J=14.3, 7.6 Hz, 2H), 7.23 (t, J=54.2 Hz, 1H), 5.25 (q, J=7.9 Hz, 1H), 4.06-3.99 (m, 2H), 2.72 (qd, J=15.6, 7.5 Hz, 2H), 1.41-1.28 (m, 21H), 1.11 (t, J=7.1 Hz, 3H).

### Step 8: Preparation of ethyl (S)-3-((tert-butoxycarbonyl)amino)-3-(5-(difluoromethyl)-4-fluoro-2′-hydroxy-6′-methyl-[1,1′-biphenyl]-3-yl)propanoate

To a stirred mixture of ethyl (S)-3-((tert-butoxycarbonyl)amino)-3-(3-(difluoromethyl)-2-fluoro-5-(4,4,5,5-tetramethyl-1,3,2-dioxaborolan-2-yl)phenyl)propanoate (1.00 g, 2.05 mmol, 1.00 equiv.) and 2-bromo-3-methylphenol (0.58 g, 3.08 mmol, 1.50 equiv.) in 1,4-dioxane (20 mL) and H₂O (1 mL) were added Pd(dppf)Cl₂·CH₂Cl₂ (0.17 g, 0.21 mmol, 0.10 equiv.) and K₂CO₃ (0.85 g, 6.16 mmol, 3.00 equiv.). The resulting mixture was stirred for 2 h at 100° C. under nitrogen atmosphere and then cooled to room temperature. The resulting mixture was concentrated under reduced pressure. The residue was purified by silica gel column chromatography (PE/EtOAc (0 to 20% gradient in 30 min)) to afford ethyl (S)-3-((tert-butoxycarbonyl)amino)-3-(5-(difluoromethyl)-4-fluoro-2′-hydroxy-6′-methyl-[1,1′-biphenyl]-3-yl)propanoate (660 mg, 68.8%) as a yellow solid. LC-MS: (ES+H, m/z) [M+H−tBu]⁺=412.1. ¹H NMR (400 MHz, DMSO-d₆) δ 9.28 (s, 1H), 7.60 (d, J=8.8 Hz, 1H), 7.45 (d, J=6.6 Hz, 1H), 7.36-7.20 (m, 2H), 7.08 (t, J=7.8 Hz, 1H), 6.76 (dd, J=11.0, 7.8 Hz, 2H), 5.31 (d, J=8.4 Hz, 1H), 4.02 (qt, J=6.9, 4.7 Hz, 2H), 2.83-2.64 (m, 2H), 1.98 (d, J=10.4 Hz, 3H), 1.34 (s, 9H), 1.15 (dt, J=21.0, 7.1 Hz, 3H).

### Step 9: Preparation of ethyl (S)-3-amino-3-(5-(difluoromethyl)-4-fluoro-2′-hydroxy-6′-methyl-[1,1′-biphenyl]-3-yl)propanoate hydrochloride

To a stirred solution of ethyl (3S)-3-[(tert-butoxycarbonyl)amino]-3-[5-(difluoromethyl)-4-fluoro-2′-hydroxy-6′-methyl-[1,1′-biphenyl]-3-yl]propanoate (600 mg, 1.28 mmol, 1.00 equiv.) in CH₂Cl₂ (5 mL) was added HCl (gas) in 1,4-dioxane (5 mL, 20.00 mmol, 15.58 equiv., 4 M) dropwise at room temperature under nitrogen atmosphere. The resulting mixture was stirred for 1 h at room temperature under nitrogen atmosphere and then concentrated to provide ethyl (S)-3-amino-3-(5-(difluoromethyl)-4-fluoro-2′-hydroxy-6′-methyl-[1,1′-biphenyl]-3-yl)propanoate hydrochloride (510 mg, crude) as a yellow solid. LC-MS: (ES+H, m/z) [M+H]⁺=368.2.

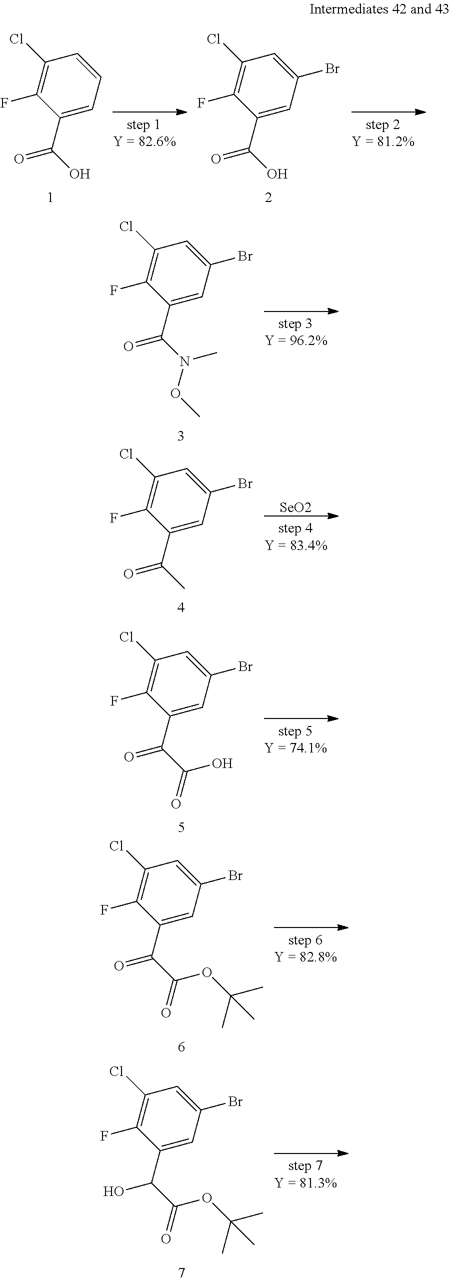

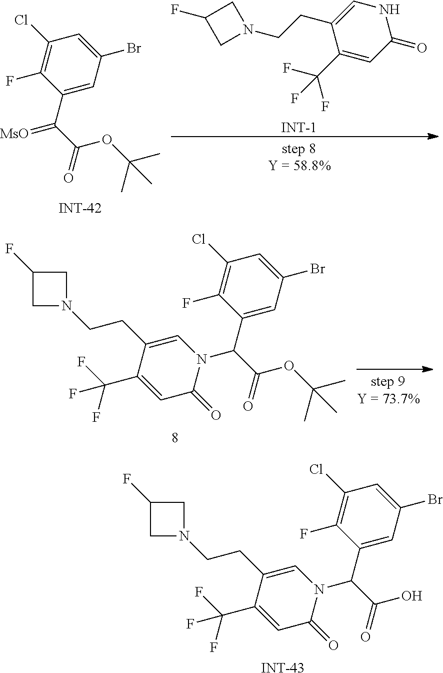

### Step 1: Preparation of 5-bromo-3-chloro-2-fluorobenzoic acid

A stirred solution of 3-chloro-2-fluorobenzoic acid (10.0 g, 57.28 mmol, 1.00 equiv.) in H₂SO₄ (50 mL) was treated with NBS (10.7 g, 60.15 mmol, 1.05 equiv.) portion-wise at 0° C. The resulting solution was stirred at 0° C. for 3 h, warmed to room temperature, and stirred overnight. The mixture was poured into ice-water (200 mL), the precipitated solids were collected by filtration and washed with water (100 mL). The resulting solid was dried in an oven under reduced pressure to afford 5-bromo-3-chloro-2-fluorobenzoic acid (12.0 g, 82.6%) as a white solid. ¹H NMR (300 MHz, DMSO-d₆) δ 8.25-8.12 (m, 1H), 7.94-7.80 (m, 1H).

### Step 2: Preparation of 5-bromo-3-chloro-2-fluoro-N-methoxy-N-methylbenzamide

To a stirred solution of 5-bromo-3-chloro-2-fluorobenzoic acid (8.00 g, 31.56 mmol, 1.00 equiv.) and HATU (14.40 g, 37.87 mmol, 1.20 equiv.) in CH₂Cl₂ (100 mL) was added DIEA (12.20 g, 94.69 mmol, 3.00 equiv.) at room temperature under nitrogen atmosphere. The resulting mixture was stirred for 30 min at 30° C. under nitrogen atmosphere. To the above mixture was added N,O-dimethylhydroxylamine hydrochloride (3.39 g, 34.72 mmol, 1.10 equiv.) at room temperature and then stirred for an additional 2 h at 30° C. The mixture was cooled to room temperature and diluted with CH₂Cl₂ (150 mL). The resulting mixture was washed with water (3×150 mL), brine (3×150 mL), dried over Na₂SO₄, filtered, and concentrated. The residue was purified by silica gel column chromatography (EtOAc/PE (0 to 25% gradient in 30 min)) to afford 5-bromo-3-chloro-2-fluoro-N-methoxy-N-methylbenzamide (7.6 g, 81.2%) as a yellow solid. LC-MS: (ES+H, m/z) [M+H]⁺=295.9. ¹H NMR (300 MHz, DMSO-d₆) δ 8.04 (dd, J=6.5, 2.4 Hz, 1H), 7.77 (dd, J=5.2, 2.5 Hz, 1H), 3.60 (s, 3H), 3.29 (s, 3H).

### Step 3: Preparation of 1-(5-bromo-3-chloro-2-fluorophenyl)ethanone

A stirred solution of 5-bromo-3-chloro-2-fluoro-N-methoxy-N-methylbenzamide (7.60 g, 25.63 mmol, 1.00 equiv.) in THF (100 mL) was added bromo(methyl)magnesium (77 mL, 276.89 mmol, 3.00 equiv., 1 M in THF) dropwise at 0° C. under nitrogen atmosphere. The resulting mixture was stirred for 2 h at room temperature and then quenched with sat. aq. NH₄Cl (80 mL) at room temperature. The resulting mixture was extracted with EtOAc (150 mL). The organic layer was washed with water (3×120 mL), dried over anhydrous Na₂SO₄, filtered and concentrated to afford 1-(5-bromo-3-chloro-2-fluorophenyl)ethanone (6.2 g, 96.2%) as a yellow solid. ¹H NMR (300 MHz, DMSO-d₆) δ 8.17 (dd, J=6.2, 2.5 Hz, 1H), 7.88 (dd, J=6.2, 2.5 Hz, 1H), 2.61 (d, J=3.9 Hz, 3H).

### Step 4: Preparation of (5-bromo-3-chloro-2-fluorophenyl)(oxo)acetic acid

A stirred solution of 1-(5-bromo-3-chloro-2-fluorophenyl)ethanone (3.00 g, 11.93 mmol, 1.00 equiv.) in pyridine (25 mL) was treated with SeO₂ (2.00 g, 17.89 mmol, 1.50 equiv.) at room temperature under nitrogen atmosphere. The resulting mixture was stirred for 2 h at 100° C. and then cooled to room temperature. The mixture was acidified to pH 1 with 3 M HCl (aq.), and extracted with EtOAc (2×200 mL). The combined organic layers were washed with water (3×150 mL), dried over anhydrous Na₂SO₄, filtered and concentrated to afford (5-bromo-3-chloro-2-fluorophenyl)(oxo)acetic acid (2.8 g, 83.4%) as a yellow solid. LC-MS: (ES−H, m/z) [M−H]⁻=278.8.

### Step 5: Preparation of tert-butyl 2-(5-bromo-3-chloro-2-fluorophenyl)-2-oxoacetate

A stirred solution of (5-bromo-3-chloro-2-fluorophenyl)(oxo)acetic acid (2.70 g, 9.59 mmol, 1.00 equiv.) in CH₂Cl₂ (30 mL) was added N,N′-diisopropyltert-butoxymethanimidamide (3.80 g, 19.19 mmol, 2.00 equiv.) at room temperature under nitrogen atmosphere. The resulting mixture was stirred for 5 h at room temperature, and then filtered. The filter cake was washed with CH₂Cl₂ (3×20 mL) and the filtrate was concentrated under reduced pressure. The residue was purified by silica gel column chromatography (EtOAc/PE (0 to 15% gradient in 20 min) to afford tert-butyl 2-(5-bromo-3-chloro-2-fluorophenyl)-2-oxoacetate (2.4 g, 74.1%) as a yellow solid. ¹H NMR (400 MHz, DMSO-d₆) δ 8.33 (dd, J=6.4, 2.5 Hz, 1H), 7.95 (dd, J=5.5, 2.5 Hz, 1H), 1.54 (s, 9H).

### Step 6: Preparation of tert-butyl 2-(5-bromo-3-chloro-2-fluorophenyl)-2-hydroxyacetate

To a stirred solution of tert-butyl 2-(5-bromo-3-chloro-2-fluorophenyl)-2-oxoacetate (2.40 g, 7.11 mmol, 1.00 equiv.) in CH₃CH₂OH (30 mL) and CH₃COOH (3 mL) was added NaBH₃CN (491 mg, 7.82 mmol, 1.10 equiv.) at room temperature under nitrogen atmosphere. After 2 h, the reaction was quenched by the addition of water (4 mL) at room temperature. The resulting mixture was diluted with water (100 mL) and extracted with EtOAc (2×80 mL). The combined organic layers were washed with water (2×120 mL), dried over anhydrous Na₂SO₄, filtered, and concentrated. The residue was purified by silica gel column chromatography (EtOAc/PE (0 to 15% gradient in 20 min) to afford tert-butyl 2-(5-bromo-3-chloro-2-fluorophenyl)-2-hydroxyacetate (2.0 g, 82.8%) as a yellow oil. LC-MS: (ES−H, m/z) [M−H]⁻=337.0.

### Step 7: Preparation of tert-butyl 2-(5-bromo-3-chloro-2-fluorophenyl)-2-(methanesulfonyloxy)acetate (Intermediate 42)

To a stirred solution of tert-butyl 2-(5-bromo-3-chloro-2-fluorophenyl)-2-hydroxyacetate (2.10 g, 6.18 mmol, 1.00 equiv.) and Et₃N (1.90 g, 18.55 mmol, 3.00 equiv.) in CH₂Cl₂ (30 mL) was added MsCl (1.70 g, 7.42 mmol, 1.20 equiv., 50% in CH₂Cl₂) dropwise at 0° C. under nitrogen atmosphere. The resulting mixture was stirred for 2 h at room temperature and then concentrated. The residue was purified by silica gel column chromatography (EtOAc/PE (0 to 20% gradient in 20 min) to afford tert-butyl 2-(5-bromo-3-chloro-2-fluorophenyl)-2-(methanesulfonyloxy)acetate (2.1 g, 81.3%) as a colorless solid. ¹H NMR (400 MHz, CDCl₃) δ7.61 (dd, J=6.3, 2.4 Hz, 1H), 7.46 (dd, J=5.5, 2.4 Hz, 1H), 6.04 (s, 1H), 3.21 (s, 3H), 1.46 (s, 9H).

### Step 8: Preparation of tert-butyl 2-(5-bromo-3-chloro-2-fluorophenyl)-2-{5-[2-(3-fluoroazetidin-1-yl)ethyl]-2-oxo-4-(trifluoromethyl)pyridin-1-yl}acetate

To a stirred solution of tert-butyl 2-(5-bromo-3-chloro-2-fluorophenyl)-2-(methanesulfonyloxy)acetate (400 mg, 0.95 mmol, 1.10 equiv.) and 5-[2-(3-fluoroazetidin-1-yl)ethyl]-4-(trifluoromethyl)-1H-pyridin-2-one (230 mg, 0.87 mmol, 1.00 equiv.) in CH₃CN (5 mL) was added K₂CO₃ (360 mg, 2.61 mmol, 3.00 equiv.) at room temperature under nitrogen atmosphere. The resulting mixture was stirred for 2 h at 80° C. under nitrogen atmosphere and then cooled to room temperature. The reaction was filtered and the filter cake was washed with CH₂Cl₂ (3×6 mL). The combined filtrates were concentrated under reduced pressure. The residue was purified by reversed-phase flash chromatography (C18 gel; mobile phase, CH₃CN in water (0.10% NH₃·H₂O), 50% to 80% gradient in 30 min; detector, UV 254 nm) to provide tert-butyl 2-(5-bromo-3-chloro-2-fluorophenyl)-2-{5-[2-(3-fluoroazetidin-1-yl)ethyl]-2-oxo-4-(trifluoromethyl)pyridin-1-yl}acetate (300 mg, 58.8%) as a colorless oil. LC-MS: (ES+H, m/z) [M+H]⁺=585.0. ¹H NMR (300 MHz, DMSO-d₆) δ 8.05 (dd, J=6.4, 2.4 Hz, 1H), 7.73 (s, 1H), 7.58 (dd, J=5.8, 2.4 Hz, 1H), 6.89 (s, 1H), 6.53 (s, 1H), 5.25-4.85 (m, 1H), 3.57-3.40 (m, 2H), 3.09-2.95 (m, 2H), 2.59-2.54 (m, 2H), 2.44 (t, J=7.2 Hz, 2H), 1.41 (s, 9H).

### Step 9: Preparation of (5-bromo-3-chloro-2-fluorophenyl)({5-[2-(3-fluoroazetidin-1-yl)ethyl]-2-oxo-4-(trifluoromethyl)pyridin-1-yl})acetic acid (Intermediate 43)

To a stirred solution of tert-butyl 2-(5-bromo-3-chloro-2-fluorophenyl)-2-{5-[2-(3-fluoroazetidin-1-yl)ethyl]-2-oxo-4-(trifluoromethyl)pyridin-1-yl}acetate (300 mg, 0.51 mmol, 1.00 equiv.) in CH₂Cl₂ (6 mL) was added CF₃COOH (3.00 mL, 40.38 mmol, 78.87 equiv.) at room temperature under nitrogen atmosphere. After 5 h the reaction was concentrated and the residue was purified by reversed-phase flash chromatography (C18 gel; mobile phase, CH₃CN in water (0.1% NH₃·H₂O), 10% to 70% gradient in 50 min; detector, UV 254 nm) to provide (5-bromo-3-chloro-2-fluorophenyl)({5-[2-(3-fluoroazetidin-1-yl)ethyl]-2-oxo-4-(trifluoromethyl)pyridin-1-yl})acetic acid (200 mg, 73.7%) as a white solid. LC-MS: (ES+H, m/z) [M+H]⁺=528.9.

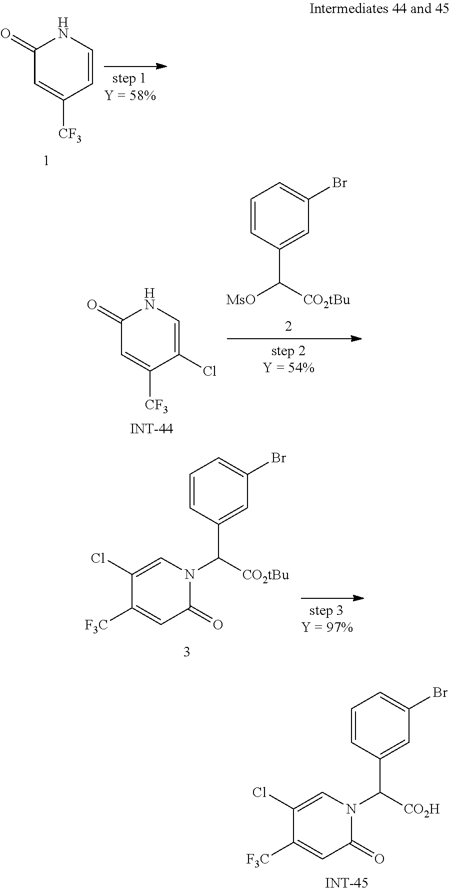

### Step 1: Preparation of 5-chloro-4-(trifluoromethyl)-1H-pyridin-2-one (Intermediate 44)

A solution of 4-(trifluoromethyl)-1H-pyridin-2-one (5.00 g, 30.66 mmol, 1.00 equiv.) and NCS (4.09 g, 30.66 mmol, 1.00 equiv.) in CHCl₃ (20 mL) was stirred for 2 h at 80° C. under nitrogen atmosphere and then cooled to room temperature. The resulting mixture was concentrated under reduced pressure. The residue was dissolved in EtOAc (100 mL). The organic layer was washed with water (3×40 mL), dried over anhydrous Na₂SO₄, filtered, and concentrated. The crude product was re-crystallized from petroleum ether/acetone (2:1 120 mL) to afford 5-chloro-4-(trifluoromethyl)-1H-pyridin-2-one (3.50 g, 57.8%) as an off-white solid. LC-MS: (ES+H, m/z) [M+H]⁺=197.95. ¹H NMR (400 MHz, DMSO-d₆) δ 12.53-12.24 (m, 1H), 8.01 (s, 1H), 6.91 (s, 1H). ¹⁹F NMR (377 MHz, DMSO-d₆) δ −64.36.

### Step 2: Preparation of tert-butyl 2-(3-bromophenyl)-2-[5-chloro-2-oxo-4-(trifluoromethyl)pyridin-1-yl]acetate

A solution of 5-chloro-4-(trifluoromethyl)-1H-pyridin-2-one (300 mg, 1.52 mmol, 1.00 equiv.), tert-butyl 2-(3-bromophenyl)-2-(methanesulfonyloxy)acetate (555 mg, 1.52 mmol, 1.00 equiv.) and K₂CO₃ (420 mg, 3.04 mmol, 2.00 equiv.) in MeCN (10 mL) was stirred for 2 h at 80° C. under nitrogen atmosphere and then cooled to room temperature. The resulting mixture was concentrated under reduced pressure. The residue was purified by silica gel column chromatography (petroleum ether/EtOAc (0% to 60% in 20 min) to afford tert-butyl 2-(3-bromophenyl)-2-[5-chloro-2-oxo-4-(trifluoromethyl)pyridin-1-yl]acetate (380 mg, 53.6%) as a white solid. LC-MS: (ES−H, m/z) [M−H]⁻=463.70. ¹H NMR (400 MHz, DMSO-d₆) δ 7.94 (s, 1H), 7.75 (t, J=1.9 Hz, 1H), 7.65 (ddd, J=7.9, 2.0, 1.1 Hz, 1H), 7.49 (dt, J=7.8, 1.5 Hz, 1H), 7.42 (t, J=7.8 Hz, 1H), 7.10 (s, 1H), 6.27 (s, 1H), 1.42 (s, 9H). ¹⁹F NMR (377 MHz, DMSO-d₆) δ −64.42.

### Step 3: Preparation of (3-bromophenyl)[5-chloro-2-oxo-4-(trifluoromethyl)pyridin-1-yl]acetic acid ((Intermediate 45)

A solution of tert-butyl 2-(3-bromophenyl)-2-[5-chloro-2-oxo-4-(trifluoromethyl)pyridin-1-yl]acetate (350 mg, 0.75 mmol, 1.00 equiv.) and trifluoroacetic acid (5 mL) in CH₂Cl₂ (5 mL) was stirred overnight at room temperature under nitrogen atmosphere and then concentrated. The residue was purified by reversed-phase flash chromatography (C18 gel; mobile phase, MeCN in water (10 mmol/L NH₄HCO₃), 30% to 100% gradient in 15 min; detector, UV 220 nm) to provide (3-bromophenyl)[5-chloro-2-oxo-4-(trifluoromethyl)pyridin-1-yl]acetic acid (300 mg, 97.4%) as a white solid. LC-MS: (ES+H, m/z) [M+H]⁺=409.85.

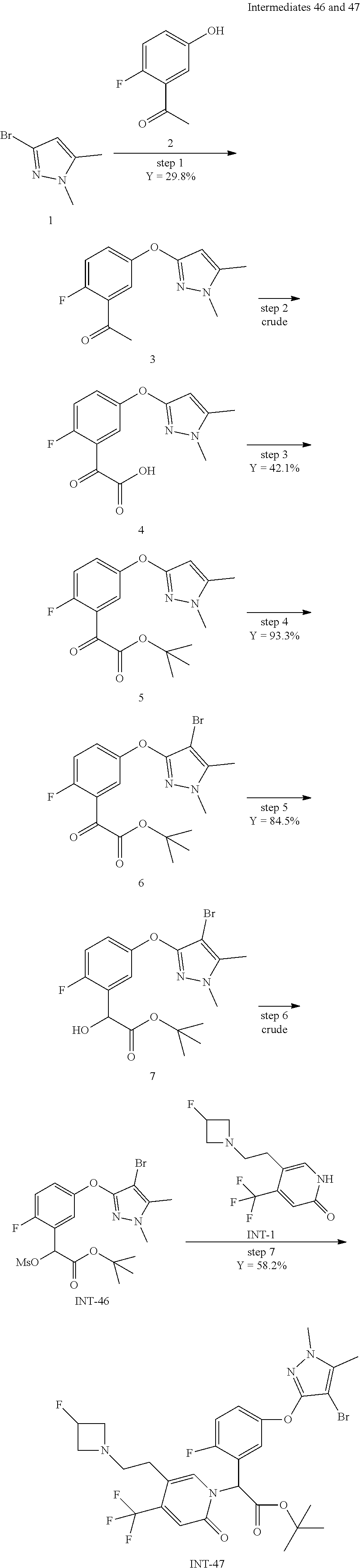

### Step 1: Preparation of 1-{5-[(1,5-dimethylpyrazol-3-yl)oxy]-2-fluorophenyl}ethanone

To a stirred solution of 1-(2-fluoro-5-hydroxyphenyl)ethanone (3.00 g, 19.46 mmol, 1.00 equiv.) and 3-bromo-1,5-dimethylpyrazole (4.50 g, 25.69 mmol, 1.32 equiv.) in dioxane (120 mL) were added CuI (1.85 g, 9.73 mmol, 0.50 equiv.), 2-(dimethylamino)acetic acid (2.01 g, 19.46 mmol, 1.00 equiv.) and Cs₂CO₃ (12.68 g, 38.92 mmol, 2.00 equiv.) in portions at room temperature under nitrogen atmosphere. The resulting mixture was stirred overnight at 120° C. under nitrogen atmosphere and then cooled to room temperature. The resulting mixture was filtered, and the filter cake was washed with EtOAc (3×100 mL). The filtrate was concentrated under reduced pressure. The residue was purified by silica gel column chromatography (PE/EtOAc (0%-40% gradient in 40 min) to afford 1-{5-[(1,5-dimethylpyrazol-3-yl)oxy]-2-fluorophenyl}ethanone (1.80 g, 29.8%) as a yellow oil. LC-MS: (ES−H, m/z) [M+H]⁺=249.3. ¹H NMR (400 MHz, CDCl₃) δ 7.57 (dd, J=5.9, 3.2 Hz, 1H), 7.31-7.26 (m, 1H), 7.08 (dd, J=10.3, 8.9 Hz, 1H), 5.57 (s, 1H), 3.67 (s, 3H), 2.62 (d, J=5.1 Hz, 3H), 2.24 (s, 3H).

### Step 2: Preparation of {5-[(1,5-dimethylpyrazol-3-yl)oxy]-2-fluorophenyl}(oxo)acetic acid

To a stirred solution of 1-{5-[(1,5-dimethylpyrazol-3-yl)oxy]-2-fluorophenyl}ethanone (1.40 g, crude) in pyridine (10 mL) was added SeO₂ (938 mg, 8.46 mmol, 1.50 equiv.) in portions at room temperature. The resulting mixture was stirred for 2 h at 100° C. under nitrogen atmosphere and then cooled to room temperature. The resulting mixture was diluted with EtOAc (150 mL), washed with 1M HCl (aq.) (3×150 mL), brine (1×100 mL), dried over anhydrous Na₂SO₄, filtered, and concentrated to provide {5-[(1,5-dimethylpyrazol-3-yl)oxy]-2-fluorophenyl}(oxo)acetic acid (1.3 g, crude) as a yellow oil. LC-MS: (ES−H, m/z) [M+H]⁺=279.0. ¹H NMR (400 MHz, DMSO-d₆) δ 7.41-7.36 (m, 2H), 7.29-7.26 (m, 1H), 5.73 (d, J=0.8 Hz, 1H), 2.23 (d, J=1.8 Hz, 3H), 1.99 (s, 3H). ¹⁹F NMR (377 MHz, DMSO-d₆) δ −119.20.

### Step 3: Preparation of tert-butyl 2-{5-[(1,5-dimethylpyrazol-3-yl)oxy]-2-fluorophenyl}-2-oxoacetate

To a stirred solution of {5-[(1,5-dimethylpyrazol-3-yl)oxy]-2-fluorophenyl}(oxo)acetic acid (1.50 g, 5.39 mmol, 1.00 equiv.) in CH₂Cl₂ (15 mL) were added N,N′-diisopropyltert-butoxymethanimidamide (2.16 g, 10.78 mmol, 2.00 equiv.) in portions at room temperature. The resulting mixture was stirred overnight at room temperature and then diluted with water (150 mL) and extracted with CH₂Cl₂ (3×100 mL). The combined organic layers were washed with brine (1×100 mL), dried over anhydrous Na₂SO₄, filtered, and concentrated. The residue was purified by silica gel column chromatography (PE/EtOAc (0%-40% gradient in 40 min) to afford tert-butyl 2-{5-[(1,5-dimethylpyrazol-3-yl)oxy]-2-fluorophenyl}-2-oxoacetate (800 mg, 42.1%) as a yellow oil. LC-MS: (ES−H, m/z) [M+H]⁺=335.0. ¹H NMR (300 MHz, CDCl₃) δ7.63 (dd, J=5.6, 3.2 Hz, 1H), 7.42-7.38 (m, 1H), 7.17-7.08 (m, 1H), 5.61 (d, J=0.8 Hz, 1H), 3.71 (s, 3H), 2.28 (d, J=0.6 Hz, 3H), 1.61 (s, 9H).

### Step 4: Preparation of tert-butyl 2-{5-[(4-bromo-1,5-dimethylpyrazol-3-yl)oxy]-2-fluorophenyl}-2-oxoacetate

To a stirred solution of tert-butyl 2-{5-[(1,5-dimethylpyrazol-3-yl)oxy]-2-fluorophenyl}-2-oxoacetate (900 mg, 2.62 mmol, 1.00 equiv.) in MeCN (15 mL) was added NBS (479 mg, 2.69 mmol, 1.00 equiv.) in portions at room temperature. The resulting mixture was stirred for 1 h at 40° C. under nitrogen atmosphere and then cooled to room temperature. The resulting mixture was diluted with EtOAc (100 mL), washed with water (2×50 mL). The organic layer was washed with brine (1×50 mL), dried over anhydrous Na₂SO₄, filtered, and concentrated. The residue was purified by silica gel column chromatography (PE/EtOAc (0%-30% gradient in 40 min) to afford tert-butyl 2-{5-[(4-bromo-1,5-dimethylpyrazol-3-yl)oxy]-2-fluorophenyl}-2-oxoacetate (1.1 g, 93.9%) as a yellow oil. LC-MS: (ES+H, m/z) [M+H]⁺=413.0. ¹H NMR (300 MHz, CDCl₃) δ7.50 (dd, J=5.5, 3.1 Hz, 1H), 7.36-7.28 (m, 1H), 7.04 (dd, J=9.8, 9.0 Hz, 1H), 3.66 (s, 3H), 2.21 (s, 3H), 1.52 (s, 9H).

### Step 5: Preparation of tert-butyl 2-{5-[(4-bromo-1,5-dimethylpyrazol-3-yl)oxy]-2-fluorophenyl}-2-hydroxyacetate

To a stirred solution of tert-butyl 2-{5-[(4-bromo-1,5-dimethylpyrazol-3-yl)oxy]-2-fluorophenyl}-2-oxoacetate (1.10 g, 2.66 mmol, 1.00 equiv.) in EtOH (12 mL) and HOAc (1.2 mL) were added NaBH₃CN (0.18 g, 2.92 mmol, 1.10 equiv.) in portions at room temperature. The resulting mixture was stirred for 1 h at room temperature and then diluted with EtOAc (100 mL). The resulting mixture was washed with water (2×50 mL), brine (1×50 mL), dried over anhydrous Na₂SO₄, filtered, and concentrated to provide tert-butyl 2-{5-[(4-bromo-1,5-dimethylpyrazol-3-yl)oxy]-2-fluorophenyl}-2-hydroxyacetate (1.1 g, crude) as a yellow solid. LC-MS: (ES+H, m/z) [M+H]⁺=415.0. ¹H NMR (300 MHz, CDCl₃) δ7.12-6.99 (m, 3H), 5.24 (s, 1H), 3.73 (s, 3H), 2.28 (s, 3H), 1.42 (s, 9H).

### Step 6: Preparation of tert-butyl 2-{5-[(4-bromo-1,5-dimethylpyrazol-3-yl)oxy]-2-fluorophenyl}-2-(methanesulfonyloxy)acetate (Intermediate 46)

To a stirred solution of tert-butyl 2-{5-[(4-bromo-1,5-dimethylpyrazol-3-yl)oxy]-2-fluorophenyl}-2-hydroxyacetate (1.10 g, crude) in DCM (15 mL) was added MsCl (0.45 g, 3.97 mmol, 1.50 equiv.) and Et₃N (0.80 g, 7.94 mmol, 3.00 equiv.) in portions at room temperature. The resulting mixture was stirred for 3 h at room temperature under nitrogen atmosphere and then diluted with water (100 mL). The resulting mixture was extracted with CH₂Cl₂ (3×150 mL). The combined organic layers were washed with brine (1×50 mL), dried over anhydrous Na₂SO₄, filtered, and concentrated to provide tert-butyl 2-{5-[(4-bromo-1,5-dimethylpyrazol-3-yl)oxy]-2-fluorophenyl}-2-(methanesulfonyloxy)acetate (1.1 g, crude) as a yellow solid. LC-MS: (ES+H, m/z) [M+H]⁺=493.0. ¹H NMR (300 MHz, CDCl₃) δ7.21-7.05 (m, 3H), 6.08 (s, 1H), 3.74 (s, 3H), 3.14 (s, 3H), 2.29 (s, 3H), 1.45 (s, 9H).

### Step 7: Preparation of tert-butyl 2-{5-[(4-bromo-1,5-dimethylpyrazol-3-yl)oxy]-2-fluorophenyl}-2-{5-[2-(3-fluoroazetidin-1-yl)ethyl]-2-oxo-4-(trifluoromethyl)pyridin-1-yl}acetate (Intermediate 47)

To a stirred solution of tert-butyl 2-{5-[(4-bromo-1,5-dimethylpyrazol-3-yl)oxy]-2-fluorophenyl}-2-(methanesulfonyloxy)acetate (1.10 g, 2.23 mmol, 1.30 equiv.) in MeCN (15 mL) were added 5-[2-(3-fluoroazetidin-1-yl)ethyl]-4-(trifluoromethyl)-1H-pyridin-2-one (0.45 g, 1.71 mmol, 1.00 equiv.) and K₂CO₃ (0.71 g, 5.14 mmol, 3.00 equiv.) dropwise at room temperature. The resulting mixture was stirred for 3 h at 80° C. under nitrogen atmosphere and then cooled to room temperature. The resulting mixture was diluted with EtOAc (100 mL) and washed with water (2×70 mL), brine (1×70 mL), dried over anhydrous Na₂SO₄, filtered, and concentrated. The residue was purified by reversed-phase flash chromatography (C18 gel; mobile phase, MeCN in water (0.1% NH₃·H₂O), 0% to 70% gradient in 30 min; detector, UV 220 nm) to provide tert-butyl 2-{5-[(4-bromo-1,5-dimethylpyrazol-3-yl)oxy]-2-fluorophenyl}-2-{5-[2-(3-fluoroazetidin-1-yl)ethyl]-2-oxo-4-(trifluoromethyl)pyridin-1-yl}acetate (700 mg, 58.2%) as a white solid. LC-MS: (ES+H, m/z) [M+H]⁺=661.2. ¹H NMR (400 MHz, CD₃OD) δ7.36 (s, 1H), 7.30-7.26 (m, 2H), 7.05-7.03 (m, 1H), 6.88 (s, 1H), 6.59 (s, 1H), 5.10-4.92 (m, 1H), 3.74 (s, 3H), 3.54-3.44 (m, 2H), 3.14-3.00 (m, 2H), 2.60-2.47 (m, 4H), 2.29 (s, 3H), 1.44 (s, 9H).

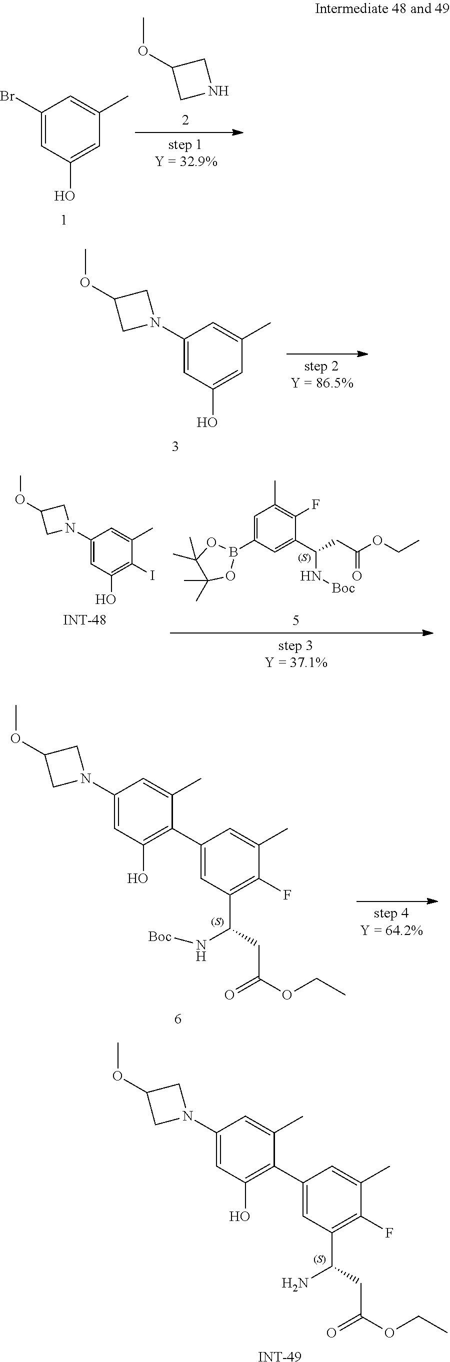

### Step 1: Preparation of 3-(3-methoxyazetidin-1-yl)-5-methylphenol

To a stirred mixture of 3-bromo-5-methylphenol (5.00 g, 26.73 mmol, 1.00 equiv.) and 3-methoxyazetidine hydrochloride (3.96 g, 32.08 mmol, 1.20 equiv.) in toluene (200 mL) were added Pd₂(dba)₃ (1.22 g, 1.34 mmol, 0.05 equiv.), XPhos (1.27 g, 2.67 mmol, 0.10 equiv.) and t-BuONa (7.71 g, 80.20 mmol, 3.00 equiv.) at room temperature under nitrogen atmosphere. The resulting mixture was stirred for 2 h at 80° C. under nitrogen atmosphere and then concentrated. The residue was purified by reversed flash chromatography (C18 gel; mobile phase, MeCN in water (0.1% HCOOH), 10% to 50% gradient in 20 min; detector, UV 220 nm) to afford 3-(3-methoxyazetidin-1-yl)-5-methylphenol (1.70 g, 32.9%) as a purple solid. LC-MS: (ES+H, m/z) [M+H]⁺=194.15.

### Step 2: Preparation of 2-iodo-5-(3-methoxyazetidin-1-yl)-3-methylphenol

To a solution of 3-(3-methoxyazetidin-1-yl)-5-methylphenol (700 mg, 3.62 mmol, 1.00 equiv.) in toluene (20 mL) was added sodium hydride (60% in oil, 290 mg, 7.24 mmol, 2.00 equiv.) at 0° C. The mixture was stirred for 1 h at 0° C. and then I₂ (873 mg, 3.44 mmol, 0.95 equiv.) in toluene (30 mL) was added at 0° C. After 1 h, the reaction mixture was quenched by water (30 mL). The mixture was acidified to pH 5 with HCl (1 M) and extracted with EtOAc (3×60 mL). The combined organic layers were washed with brine (3×50 mL), dried over anhydrous Na₂SO₄, filtered, and concentrated to provide 2-iodo-5-(3-methoxyazetidin-1-yl)-3-methylphenol (1 g, 86.5%) as a purple solid. LC-MS: (ES+H, m/z) [M+H]⁺=319.90. ¹H NMR (400 MHz, DMSO-d₆) δ 9.97 (s, 1H), 5.99 (d, J=2.5 Hz, 1H), 5.86 (d, J=2.5 Hz, 1H), 4.31 (tt, J=6.2, 4.3 Hz, 1H), 4.05-3.95 (m, 2H), 3.56 (dd, J=8.1, 4.2 Hz, 2H), 3.27 (s, 3H), 2.29 (s, 3H).

### Step 3: Preparation of ethyl (3S)-3-[(tert-butoxycarbonyl)amino]-3-[4-fluoro-2′-hydroxy-4′-(3-methoxyazetidin-1-yl)-5,6′-dimethyl-[1,1′-biphenyl]-3-yl]propanoate

A mixture of 2-iodo-5-(3-methoxyazetidin-1-yl)-3-methylphenol (500 mg, 1.57 mmol, 1.00 equiv.), ethyl (3S)-3-[(tert-butoxycarbonyl)amino]-3-[2-fluoro-3-methyl-5-(4,4,5,5-tetramethyl-1,3,2-dioxaborolan-2-yl)phenyl]propanoate (778 mg, 1.72 mmol, 1.10 equiv.), K₂CO₃ (433 mg, 3.13 mmol, 2.00 equiv.) and Pd(dppf)Cl₂ (115 mg, 0.16 mmol, 0.10 equiv.) in 1,4-dioxane (20 mL) and H₂O (1 mL) was stirred for 2 h at 100° C. under nitrogen atmosphere and then cooled to room temperature. The resulting mixture was concentrated under reduced pressure. The residue was purified by silica gel column chromatography (petroleum ether/EtOAc (0% to 50% in 20 min) to afford ethyl (3S)-3-[(tert-butoxycarbonyl)amino]-3-[4-fluoro-2′-hydroxy-4′-(3-methoxyazetidin-1-yl)-5,6′-dimethyl-[1,1′-biphenyl]-3-yl]propanoate (300 mg, 37.1%) as a brown oil. LC-MS: (ES+H, m/z) [M+H]⁺=517.20. ¹H NMR (300 MHz, DMSO-d₆) δ 8.88 (s, 1H), 7.50 (d, J=8.9 Hz, 1H), 6.96 (dd, J=25.0, 7.0 Hz, 2H), 5.87-5.82 (m, 1H), 5.27 (d, J=8.1 Hz, 1H), 4.30 (t, J=5.1 Hz, 1H), 4.02 (ddd, J=14.3, 7.6, 5.9 Hz, 4H), 3.55-3.51 (m, 2H), 2.64 (d, J=7.1 Hz, 2H), 2.22 (d, J=1.9 Hz, 3H), 1.89 (s, 3H), 1.34 (s, 9H), 1.14 (t, J=7.2 Hz, 3H). ¹⁹F NMR (282 MHz, DMSO-d₆) δ −128.34.

### Step 4: Preparation of ethyl (3S)-3-amino-3-[4-fluoro-2′-hydroxy-4′-(3-methoxyazetidin-1-yl)-5,6′-dimethyl-[1,1′-biphenyl]-3-yl]propanoate

A solution of ethyl (3S)-3-[(tert-butoxycarbonyl)amino]-3-[4-fluoro-2′-hydroxy-4′-(3-methoxyazetidin-1-yl)-5,6′-dimethyl-[1,1′-biphenyl]-3-yl]propanoate (290 mg, 0.56 mmol, 1.00 equiv.) and trifluoroacetic acid (5 mL) in CH₂Cl₂ (5 mL) was stirred for 2 h at room temperature under nitrogen atmosphere and then concentrated. The residue was purified by reversed-phase flash chromatography (C18 gel; mobile phase, MeCN in water (0.1% NH₃·H₂O), 0% to 100% gradient in 20 min; detector, UV 220 nm) to provide ethyl (3S)-3-amino-3-[4-fluoro-2′-hydroxy-4′-(3-methoxyazetidin-1-yl)-5,6′-dimethyl-[1,1′-biphenyl]-3-yl]propanoate (150 mg, 64.2%) as a purple colored oil. LC-MS: (ES+H, m/z) [M+H]⁺=417.15.

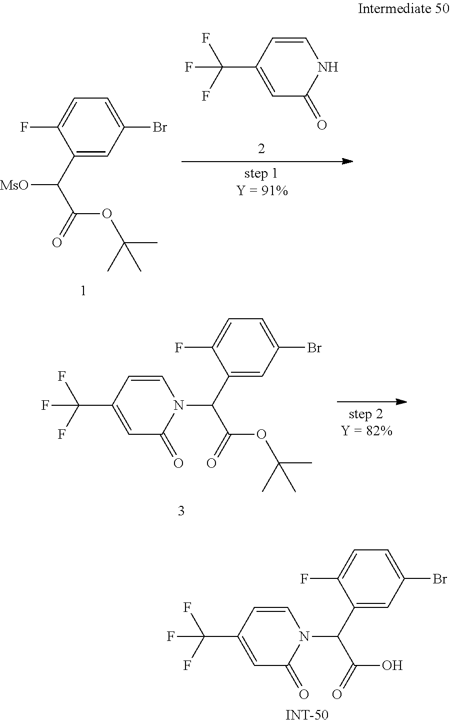

### Step 1: Preparation of tert-butyl 2-(5-bromo-2-fluorophenyl)-2-[2-oxo-4-(trifluoromethyl)pyridin-1-yl]acetate

A mixture of tert-butyl 2-(5-bromo-2-fluorophenyl)-2-(methanesulfonyloxy)acetate (3.00 g, 7.83 mmol, 1.00 equiv.), 4-(trifluoromethyl)-1H-pyridin-2-one (1.28 g, 7.83 mmol, 1.00 equiv.) and K₂CO₃ (2.16 g, 15.66 mmol, 2.00 equiv.) in MeCN (60 mL) was stirred for 2 h at 80° C. under nitrogen atmosphere and then cooled to room temperature. The resulting mixture was diluted with water (100 mL) and extracted with EtOAc (3×100 mL). The combined organic layers were washed with brine (3×100 mL), dried over anhydrous Na₂SO₄, filtered, and concentrated. The residue was purified by silica gel column chromatography (petroleum ether/EtOAc (0% to 50% in 20 min) to afford tert-butyl 2-(5-bromo-2-fluorophenyl)-2-[2-oxo-4-(trifluoromethyl)pyridin-1-yl]acetate (3.20 g, 91.4%) as a colorless oil. LC-MS: (ES+H, m/z) [M+H]⁺=450.05. ¹H NMR (300 MHz, DMSO-d₆) δ 7.79-7.68 (m, 2H), 7.65 (dd, J=6.5, 2.5 Hz, 1H), 7.34 (dd, J=10.1, 8.8 Hz, 1H), 6.94-6.90 (m, 1H), 6.57 (s, 1H), 6.52 (dd, J=7.3, 2.1 Hz, 1H), 1.41 (s, 9H). ¹⁹F NMR (282 MHz, DMSO-d₆) δ −65.49, −116.51.

### Step 2: Preparation of (5-bromo-2-fluorophenyl)[2-oxo-4-(trifluoromethyl)pyridin-1-yl]acetic acid

To a stirred solution of tert-butyl 2-(5-bromo-2-fluorophenyl)-2-[2-oxo-4-(trifluoromethyl)pyridin-1-yl]acetate (2.00 g, 4.442 mmol, 1.00 equiv.) in CH₂Cl₂ (25 mL) was added trifluoroacetic acid (25 mL) dropwise at 0° C. under nitrogen atmosphere. The resulting mixture was stirred for 4 h at room temperature and then concentrated under reduced pressure. The residue was purified by reversed-phase flash chromatography (C18 gel; mobile phase, MeCN in water (0.1% NH₃·H₂O), 0% to 100% gradient in 20 min; detector, UV 220 nm) to provide (5-bromo-2-fluorophenyl)[2-oxo-4-(trifluoromethyl)pyridin-1-yl]acetic acid) as a brown oil (1.40 g, 82.4%). LC-MS: (ES+H, m/z) [M+H]⁺=393.80.

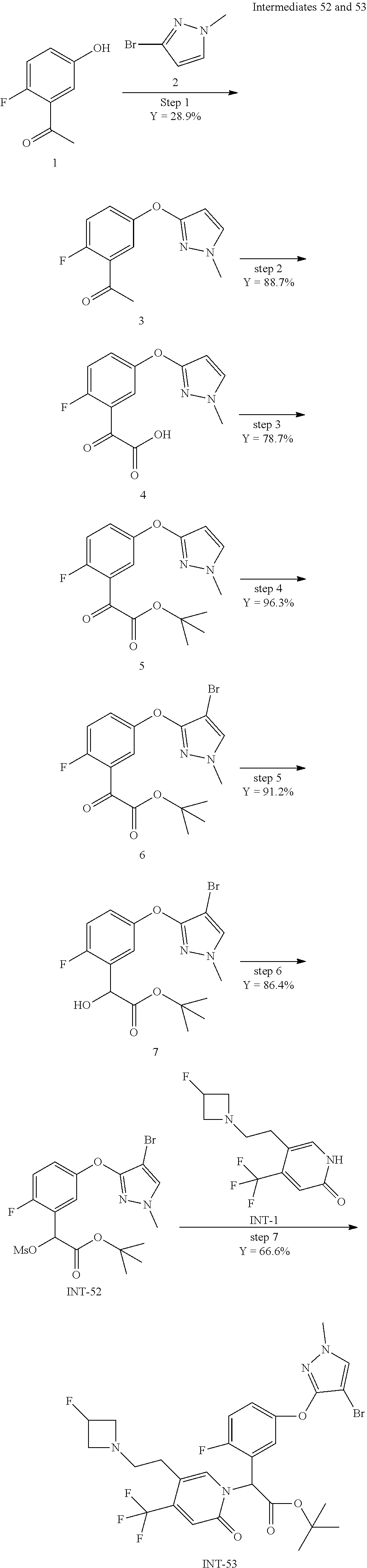

### Step 1: Preparation of 1-{2-fluoro-5-[(1-methylpyrazol-3-yl)oxy]phenyl}ethanone

To a stirred solution of 1-(2-fluoro-5-hydroxyphenyl)ethanone (5.00 g, 32.45 mmol, 1.00 equiv.) and 3-bromo-1-methylpyrazole (6.80 g, 42.15 mmol, 1.30 equiv.) in dioxane (150 mL) were added 2-(dimethylamino)acetic acid (3.34 g, 32.44 mmol, 1.00 equiv.), CuI (3.10 g, 16.20 mmol, 0.50 equiv.) and Cs₂CO₃ (21.15 g, 64.88 mmol, 2.00 equiv.) at room temperature under nitrogen atmosphere. The resulting mixture was stirred overnight at 120° C. under nitrogen atmosphere. The resulting mixture was cooled to room temperature, and filtered. The filter cake was washed with EtOAc (3×100 mL) and the combined organic layers were concentrated under reduced pressure. The residue was purified by silica gel column chromatography (petroleum ether/EtOAc (10% to 40% in 20 min) to afford 1-{2-fluoro-5-[(1-methylpyrazol-3-yl)oxy]phenyl}ethanone (2.20 g, 28.9%) as a yellow liquid. ¹H NMR (300 MHz, DMSO-d₆) δ 7.67 (d, J=2.3 Hz, 1H), 7.44-7.34 (m, 3H), 5.87 (d, J=2.3 Hz, 1H), 3.75 (s, 3H), 2.58 (d, J=4.5 Hz, 3H).

### Step 2: Preparation of {2-fluoro-5-[(1-methylpyrazol-3-yl)oxy]phenyl}(oxo)acetic acid

A solution of 1-{2-fluoro-5-[(1-methylpyrazol-3-yl)oxy]phenyl}ethanone (2.20 g, 9.39 mmol, 1.00 equiv.) and SeO₂ (2.08 g, 18.78 mmol, 2.00 equiv.) in pyridine (30 mL) was stirred for 2 h at 100° C. under nitrogen atmosphere and then cooled to room temperature and concentrated. The residue was acidified to pH 1 with HCl (3 M, aq.) and extracted with EtOAc (3×200 mL). The combined organic layers were washed with brine (1×200 mL), dried over anhydrous Na₂SO₄, filtered, and concentrated to afford {2-fluoro-5-[(1-methylpyrazol-3-yl)oxy]phenyl}(oxo)acetic acid (2.20 g, 88.7%) which was used directly in the next step.

### Step 3: Preparation of tert-butyl 2-{2-fluoro-5-[(1-methylpyrazol-3-yl)oxy]phenyl}-2-oxoacetate

A stirred solution of {2-fluoro-5-[(1-methylpyrazol-3-yl)oxy]phenyl}(oxo)acetic acid (2.20 g, 8.33 mmol, 1.00 equiv.) in CH₂Cl₂ (25 mL) was added N,N′-diisopropyltert-butoxymethanimidamide (3.34 g, 16.65 mmol, 2.00 equiv.) at room temperature under nitrogen atmosphere. The resulting mixture was stirred overnight at room temperature and then diluted with CH₂Cl₂ (100 mL). The resulting mixture was filtered and the filter cake was washed with CH₂Cl₂ (3×100 mL). The combined filtrates were concentrated under reduced pressure and the residue was purified by silica gel column chromatography (petroleum ether/EtOAc (20% to 50% in 30 min)) to afford tert-butyl 2-{2-fluoro-5-[(1-methylpyrazol-3-yl)oxy]phenyl}-2-oxoacetate (2.10 g, 78.7%) as a light yellow oil. LC-MS: (ES+H, m/z) [M+H]⁺=321.1. ¹H NMR (300 MHz, DMSO-d₆) δ 7.69 (d, J=2.3 Hz, 1H), 7.61-7.58 (m, 1H), 7.49-7.43 (m, 2H), 5.94 (d, J=2.4 Hz, 1H), 3.76 (s, 3H), 1.53 (s, 9H). ¹⁹F NMR (282 MHz, DMSO-d₆) δ −118.67.

### Step 4: Preparation of tert-butyl 2-{5-[(4-bromo-1-methylpyrazol-3-yl)oxy]-2-fluorophenyl}-2-oxoacetate

To a stirred solution of tert-butyl 2-{2-fluoro-5-[(1-methylpyrazol-3-yl)oxy]phenyl}-2-oxoacetate (2.05 g, 6.40 mmol, 1.00 equiv.) and NBS (1.14 g, 6.40 mmol, 1.00 equiv.) in CH₃CN (10 mL) were stirred for 1 h at 60° C. under nitrogen atmosphere. The resulting mixture was diluted with EtOAc (500 mL) and washed with water (2×300 mL), sat. aq. NaCl (1×300 mL), dried over anhydrous Na₂SO₄, filtered, and concentrated. The residue was purified by silica gel column chromatography (petroleum ether/EtOAc (15%-50% in 30 min)) to afford tert-butyl 2-{5-[(4-bromo-1-methylpyrazol-3-yl)oxy]-2-fluorophenyl}-2-oxoacetate (2.46 g, 96.3%) as a white solid. LC-MS: (ES+H, m/z) [M+H]⁺=399.0. ¹H NMR (300 MHz, DMSO-d₆) δ 8.02 (s, 1H), 7.62-7.59 (m, 1H), 7.51 (dd, J=10.1, 9.1 Hz, 1H), 7.41 (dd, J=5.5, 3.1 Hz, 1H), 3.78 (s, 3H), 1.54 (s, 9H). ¹⁹F NMR (282 MHz, DMSO-d₆) δ −118.18.

### Step 5: Preparation of tert-butyl 2-{5-[(4-bromo-1-methylpyrazol-3-yl)oxy]-2-fluorophenyl}-2-hydroxyacetate

To a stirred mixture of tert-butyl 2-{5-[(4-bromo-1-methylpyrazol-3-yl)oxy]-2-fluorophenyl}-2-oxoacetate (2.40 g, 6.01 mmol, 1.00 equiv.) in EtOH (25 mL) and AcOH (2.5 mL) was added NaBH₃CN (416 mg, 6.61 mmol, 1.10 equiv.) at room temperature. The resulting mixture was stirred for 2 h at room temperature, and quenched with water at room temperature. The resulting mixture was diluted with water (300 mL) and extracted with EtOAc (3×200 mL). The combined organic layers were washed with brine (500 mL), dried over anhydrous Na₂SO₄, filtered, and concentrated. The residue was purified by silica gel column chromatography (petroleum ether/EtOAc (15%-40% in 40 min) to afford tert-butyl 2-{5-[(4-bromo-1-methylpyrazol-3-yl)oxy]-2-fluorophenyl}-2-hydroxyacetate (2.20 g, 91.2%) as a colorless oil. LC-MS: (ES+H, m/z) [M+H]⁺=401.1. ¹H NMR (300 MHz, DMSO-d₆) δ 7.97 (s, 1H), 7.97-7.16 (m, 1H), 7.09-7.01 (m, 2H), 6.13 (d, J=5.9 Hz, 1H), 5.16 (d, J=5.9 Hz, 1H), 3.76 (s, 3H), 1.34 (s, 9H). ¹⁹F NMR (282 MHz, DMSO-d₆) δ −125.24.

### Step 6: Preparation of tert-butyl 2-{5-[(4-bromo-1-methylpyrazol-3-yl)oxy]-2-fluorophenyl}-2-(methanesulfonyloxy)acetate

To a stirred solution of tert-butyl 2-{5-[(4-bromo-1-methylpyrazol-3-yl)oxy]-2-fluorophenyl}-2-hydroxyacetate (2.20 g, 5.48 mmol, 1.00 equiv.) and Et₃N (1.66 g, 16.45 mmol, 3.00 equiv.) in CH₂Cl₂ (20 mL) was added MsCl (1.51 g, 6.58 mmol, 1.20 equiv.) dropwise at 0° C. under nitrogen atmosphere. The resulting mixture was stirred for 2 h at room temperature and then quenched with water (1 mL) at room temperature and concentrated. The residue was purified by silica gel column chromatography (petroleum ether/EtOAc (20%-50% in 30 min)) to afford tert-butyl 2-{5-[(4-bromo-1-methylpyrazol-3-yl)oxy]-2-fluorophenyl}-2-(methanesulfonyloxy)acetate (2.27 g, 86.4%) as a white solid. LC-MS: (ES+H, m/z) [M+H]⁺=479.0.

### Step 7: Preparation of tert-butyl 2-{5-[(4-bromo-1-methylpyrazol-3-yl)oxy]-2-fluorophenyl}-2-{5-[2-(3-fluoroazetidin-1-yl)ethyl]-2-oxo-4-(trifluoromethyl)pyridin-1-yl}acetate

To a stirred mixture of tert-butyl 2-{5-[(4-bromo-1-methylpyrazol-3-yl)oxy]-2-fluorophenyl}-2-(methanesulfonyloxy)acetate (500 mg, 1.04 mmol, 1.00 equiv.) and 5-[2-(3-fluoroazetidin-1-yl)ethyl]-4-(trifluoromethyl)-1H-pyridin-2-one (276 mg, 1.04 mmol, 1.00 equiv.) in CH₃CN (10 mL) was added K₂CO₃ (433 mg, 3.13 mmol, 3.00 equiv.) at room temperature. The resulting mixture was stirred for 2 h at 80° C. under nitrogen atmosphere and then cooled to room temperature and diluted with EtOAc (5 mL). The resulting mixture was filtered and the filter cake was washed with EtOAc (3×5 mL). The combined organic layers were concentrated under reduced pressure and the crude product was purified by reversed-phase flash chromatography (C18 gel; mobile phase, CH₃CN in water (0.1% NH₃·H₂O), 40% to 100% gradient in 30 min; detector, UV 254 nm) to provide tert-butyl 2-{5-[(4-bromo-1-methylpyrazol-3-yl)oxy]-2-fluorophenyl}-2-{5-[2-(3-fluoroazetidin-1-yl)ethyl]-2-oxo-4-(trifluoromethyl)pyridin-1-yl}acetate (450 mg, 66.6%) as a colorless oil. LC-MS: (ES+H, m/z) [M+H]⁺=647.1.

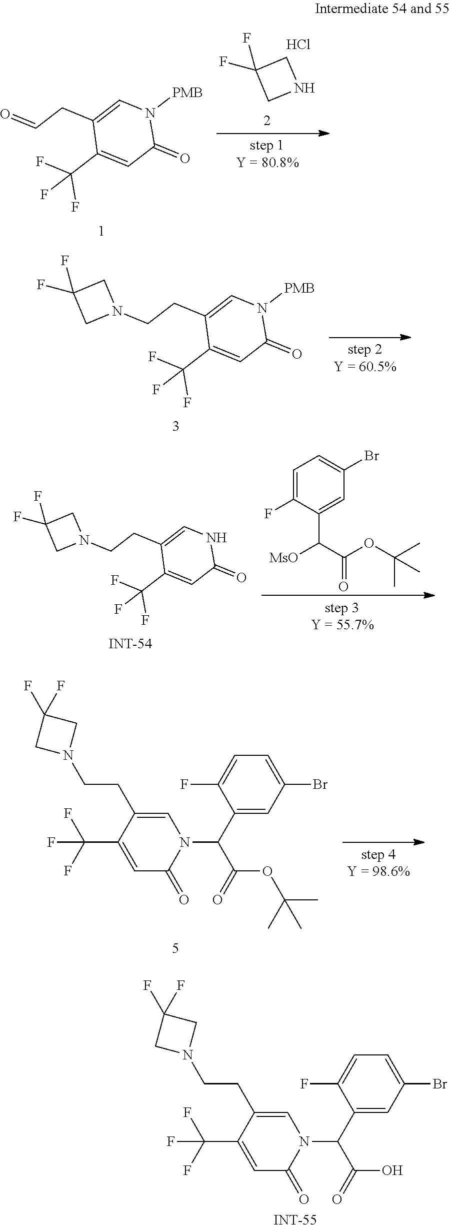

### Step 1: Preparation of 5-[2-(3,3-difluoroazetidin-1-yl)ethyl]-1-[(4-methoxyphenyl)methyl]-4-(trifluoromethyl)pyridin-2-one

To a stirred mixture of 2-{1-[(4-methoxyphenyl)methyl]-6-oxo-4-(trifluoromethyl)pyridin-3-yl}acetaldehyde (2.00 g, 6.14 mmol, 1.00 equiv.) and 3,3-difluoroazetidine hydrochloride (572 mg, 6.14 mmol, 1.00 equiv.) in EtOH (20 mL) were added CH₃COOH (36 mg, 0.61 mmol, 1.00 equiv.) at room temperature under nitrogen atmosphere. The resulting mixture was stirred for 1 h at room temperature under nitrogen atmosphere. To the above mixture was added NaBH₃CN (772 mg, 12.29 mmol, 2.00 equiv.) in portions over 2 min at room temperature. The resulting mixture was stirred for an additional 2 h at room temperature and then quenched by the addition of ice water (20 mL) at room temperature. The resulting mixture was extracted with EtOAc (2×100 mL) and the combined organic layers were washed with brine (2×100 mL), dried over anhydrous Na₂SO₄, filtered, and concentrated. The residue was purified by silica gel column chromatography (CH₂Cl₂/MeOH (0˜5%, 20 min) to afford 5-[2-(3,3-difluoroazetidin-1-yl)ethyl]-1-[(4-methoxyphenyl)methyl]-4-(trifluoromethyl)pyridin-2-one (2 g, 80.8%) as a yellow oil. LC-MS: (ES+H, m/z) [M+H]⁺=403.0. ¹H NMR (400 MHz, DMSO-d₆) δ 7.98 (s, 1H), 7.35-7.30 (m, 2H), 6.92-6.87 (m, 2H), 6.75 (s, 1H), 5.04 (s, 2H), 3.72 (s, 3H), 3.57 (t, J=12.5 Hz, 4H), 2.66 (t, J=7.2 Hz, 2H), 2.45 (t, J=7.2 Hz, 2H). ¹⁹F NMR (377 MHz, DMSO-d₆) δ −62.30, −97.86.

### Step 2: Preparation of 5-[2-(3,3-difluoroazetidin-1-yl)ethyl]-4-(trifluoromethyl)-1H-pyridin-2-one

To a stirred mixture of 5-[2-(3,3-difluoroazetidin-1-yl)ethyl]-1-[(4-methoxyphenyl)methyl]-4-(trifluoromethyl)pyridin-2-one (2.00 g, 4.97 mmol, 1.00 equiv.) in TFA (10 mL) at 0° C. under nitrogen atmosphere. The resulting mixture was stirred overnight at 80° C. under nitrogen atmosphere and then cooled to room temperature and then concentrated. The residue was purified by reversed-phase flash chromatography (C18 gel; mobile phase, MeCN in water (10 mmol/L NH₄HCO₃), 10% to 50% gradient in 20 min; detector, UV 254/220 nm) to afford 5-[2-(3,3-difluoroazetidin-1-yl)ethyl]-4-(trifluoromethyl)-1H-pyridin-2-one (850 mg, 60.5%) as a yellow solid. LC-MS: (ES+H, m/z) [M+H]⁺=282.9. ¹H NMR (400 MHz, DMSO-d₆) δ 12.12 (s, 1H), 7.56 (s, 1H), 6.68 (s, 1H), 3.58 (t, J=12.5 Hz, 4H), 2.66 (t, J=7.4 Hz, 2H), 2.45 (t, J=7.4 Hz, 2H). ¹⁹F NMR (377 MHz, DMSO-d₆) δ −62.87, −97.55.

### Step 3: Preparation of tert-butyl 2-(5-bromo-2-fluorophenyl)-2-{5-[2-(3,3-difluoroazetidin-1-yl)ethyl]-2-oxo-4-(trifluoromethyl)pyridin-1-yl}acetate

To a stirred mixture of 5-[2-(3,3-difluoroazetidin-1-yl)ethyl]-4-(trifluoromethyl)-1H-pyridin-2-one (800 mg, 2.83 mmol, 1.00 equiv.) and tert-butyl 2-(5-bromo-2-fluorophenyl)-2-(methanesulfonyloxy)acetate (1.00 g, 2.83 mmol, 1.00 equiv.) in MeCN (5 mL) was added K₂CO₃ (783 mg, 5.67 mmol, 2.00 equiv.) at room temperature under nitrogen atmosphere. The resulting mixture was stirred for 2 h at 60° C. under nitrogen atmosphere and then cooled to room temperature. The resulting mixture was diluted with water (10 mL) and extracted with EtOAc (2×50 mL). The combined organic layers were washed with brine (2×50 mL), dried over anhydrous Na₂SO₄, filtered, and concentrated. The residue was purified by reversed-phase flash chromatography (C18 gel; mobile phase, MeCN in water (10 mmol/L NH₄HCO₃), 50% to 70% gradient in 20 min; detector, UV 254/220 nm) to afford tert-butyl 2-(5-bromo-2-fluorophenyl)-2-{5-[2-(3,3-difluoroazetidin-1-yl)ethyl]-2-oxo-4-(trifluoromethyl)pyridin-1-yl}acetate (900 mg, 55.7%) as a yellow oil. LC-MS: (ES+H, m/z) [M+H]⁺=568.9. ¹H NMR (400 MHz, DMSO-d₆) δ 7.75-7.68 (m, 1H), 7.68-7.58 (m, 2H), 7.33 (t, J=10.0, 8.8 Hz, 1H), 6.89 (s, 1H), 6.53 (s, 1H), 3.49 (td, J=12.5, 2.8 Hz, 4H), 2.63 (p, J=6.0, 5.4 Hz, 2H), 2.47 (t, J=6.9 Hz, 2H), 1.41 (s, 9H). ¹⁹F NMR (377 MHz, DMSO-d₆) δ −62.70, −97.58, −116.58.

### Step 4: Preparation of (5-bromo-2-fluorophenyl)({5-[2-(3,3-difluoroazetidin-1-yl)ethyl]-2-oxo-4-(trifluoromethyl)pyridin-1-yl})acetic acid

To a stirred mixture of tert-butyl 2-(5-bromo-2-fluorophenyl)-2-{5-[2-(3,3-difluoroazetidin-1-yl)ethyl]-2-oxo-4-(trifluoromethyl)pyridin-1-yl}acetate (900 mg, 1.58 mmol, 1.00 equiv.) in CH₂C₁₂ (10 mL) was added CF₃COOH (10 mL) at room temperature under nitrogen atmosphere. The resulting mixture was stirred for 2 h at room temperature and then concentrated. The residue was purified by reversed-phase flash chromatography (C18 gel; mobile phase, MeCN in water (0.1% NH₃·H₂O), 10% to 50% gradient in 20 min; detector, UV 254/220 nm) to afford (5-bromo-2-fluorophenyl)({5-[2-(3,3-difluoroazetidin-1-yl)ethyl]-2-oxo-4-(trifluoromethyl)pyridin-1-yl})acetic acid (800 mg, 98.6%) as colorless oil. LC-MS: (ES+H, m/z) [M+H]⁺=513.0. ¹H NMR (400 MHz, DMSO-d₆) δ 7.63-7.52 (m, 3H), 7.18-7.13 (m, 1H), 6.71 (s, 1H), 6.36 (s, 1H), 3.47 (t, J=12.6 Hz, 4H), 2.63-2.53 (m, 2H), 2.47-2.39 (m, 2H). ¹⁹F NMR (377 MHz, DMSO-d₆) δ −62.45, −97.35, −116.87.

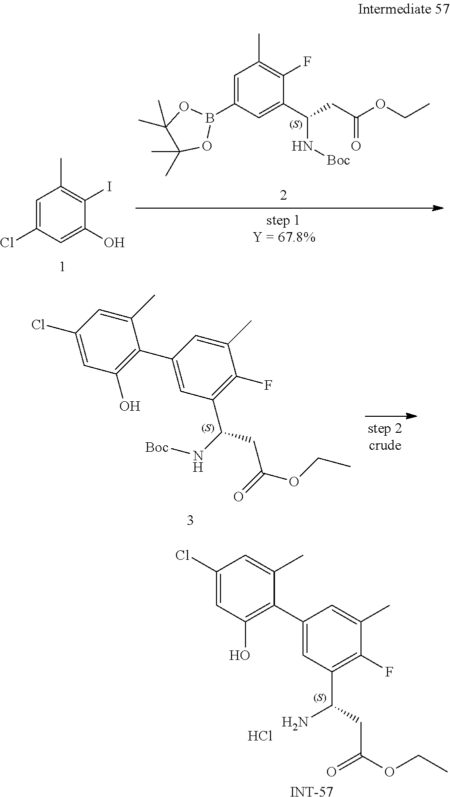

### Step 1: Preparation of ethyl (3S)-3-[(tert-butoxycarbonyl)amino]-3-{4′-chloro-4-fluoro-2′-hydroxy-5,6′-dimethyl-[1,1′-biphenyl]-3-yl}propanoate

To a stirred mixture of 5-chloro-2-iodo-3-methylphenol (2.14 g, 7.98 mmol, 1.20 equiv.) and ethyl (3S)-3-[(tert-butoxycarbonyl)amino]-3-[2-fluoro-3-methyl-5-(4,4,5,5-tetramethyl-1,3,2-dioxaborolan-2-yl)phenyl]propanoate (3.00 g, 6.65 mmol, 1.00 equiv.) in dioxane (20 mL) and water (1 mL) was added Pd(dppf)Cl₂·CH₂Cl₂ (542 mg, 0.66 mmol, 0.10 equiv.) and K₂CO₃ (2.76 g, 19.94 mmol, 3.00 equiv.) at room temperature. The resulting mixture was stirred for 2 h at 100° C. under nitrogen atmosphere and then cooled to room temperature and diluted with water (100 mL). The aqueous layer was extracted with EtOAc (3×100 mL). The combined organic layers were washed with brine (3×100 mL), dried over anhydrous Na₂SO₄, filtered, and concentrated. The residue was purified by silica gel column chromatography (PE/EtOAc (0% to 20% in 30 min)) to afford ethyl (3S)-3-[(tert-butoxycarbonyl)amino]-3-{4′-chloro-4-fluoro-2′-hydroxy-5,6′-dimethyl-[1,1′-biphenyl]-3-yl}propanoate (2.1 g, 67.8%) as a yellow oil. LC-MS: (ES+H, m/z) [M+H−tBu]⁺=410.2. ¹H NMR (400 MHz, DMSO-d₆) δ 9.63 (s, 1H), 7.49 (d, J=9.0 Hz, 1H), 7.07-6.93 (m, 2H), 6.83-6.76 (m, 2H), 5.27 (d, J=7.7 Hz, 1H), 4.03 (dt, J=7.0, 3.4 Hz, 2H), 2.65 (d, J=7.2 Hz, 2H), 2.24 (d, J=1.9 Hz, 3H), 1.94 (s, 3H), 1.33 (s, 9H), 1.13 (t, J=7.1 Hz, 3H). ¹⁹F NMR (377 MHz, DMSO-d₆) δ −126.88.

### Step 2: Preparation of ethyl (3S)-3-amino-3-{4′-chloro-4-fluoro-2′-hydroxy-5,6′-dimethyl-[1,1′-biphenyl]-3-yl}propanoate hydrochloride

To a stirred mixture of ethyl (3S)-3-[(tert-butoxycarbonyl)amino]-3-{4′-chloro-4-fluoro-2′-hydroxy-5,6′-dimethyl-[1,1′-biphenyl]-3-yl}propanoate (1.00 g, 2.12 mmol, 1.00 equiv.) and in CH₂Cl₂ (2 mL) was added HCl (gas) in 1,4-dioxane (8 mL, 4 M) dropwise at room temperature under nitrogen atmosphere. The resulting mixture was stirred for 2 h at room temperature and then concentrated to provide ethyl (3S)-3-amino-3-{4′-chloro-4-fluoro-2′-hydroxy-5,6′-dimethyl-[1,1′-biphenyl]-3-yl}propanoate hydrochloride (800 mg, crude) as a colorless oil. LC-MS: (ES+H, m/z) [M+H]⁺=366.1.

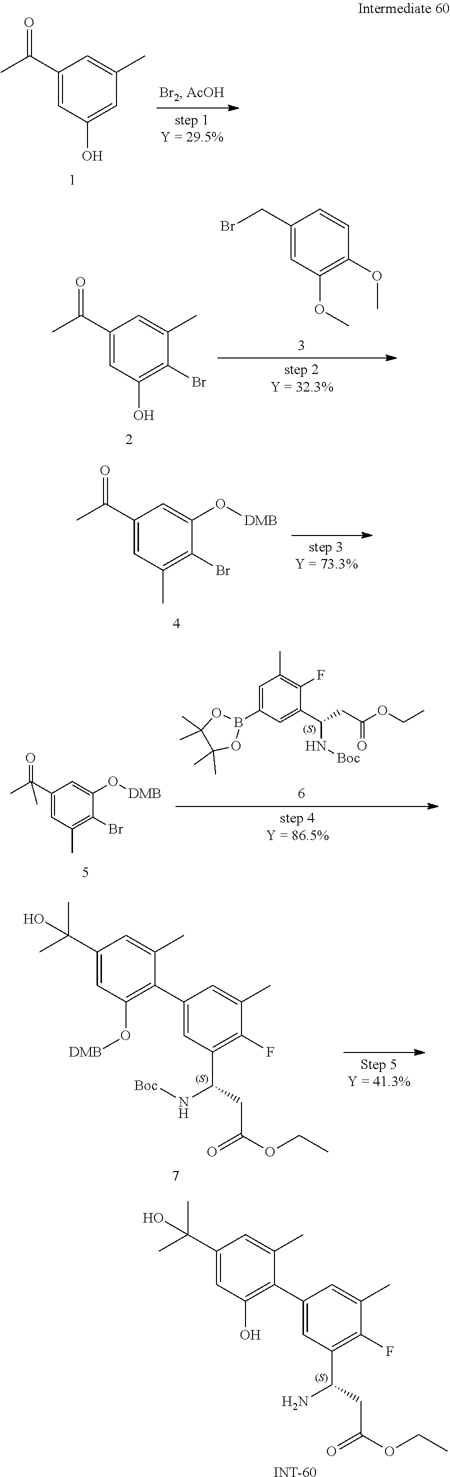

### Step 1: Preparation of 1-(4-bromo-3-hydroxy-5-methylphenyl)ethanone

To a stirred mixture of 1-(3-hydroxy-5-methylphenyl)ethanone (4.00 g, 26.63 mmol, 1.00 equiv.) in CH₃COOH (120 mL) were added Br₂ (1.5 mL, 26.63 mmol, 1.00 equiv.) in CH₃COOH (120 mL) and H₂O (13.6 mL) dropwise at 0° C. under nitrogen atmosphere. The resulting mixture was stirred for 2 h at room temperature under nitrogen atmosphere, diluted with EtOAc (1 L), and washed with sat. Na₂S₂O₃ (aq.) (1×500 mL), sat. NaHCO₃ (aq.) (1×500 mL) and concentrated. The residue was purified by reversed-phase flash chromatography (C18 gel; mobile phase, MeCN in water (0.1% NH₃·H₂O), 0% to 10% gradient in 30 min; detector, UV 254 nm) to afford 1-(4-bromo-3-hydroxy-5-methylphenyl)ethanone (1.8 g, 29.5%, crude) as a yellow oil. LC-MS: (ES−H, m/z) [M−H]⁻=226.9. ¹H NMR (300 MHz, CDCl₃) δ 7.31-7.20 (m, 2H), 2.51 (s, 3H), 2.28 (s, 3H).

### Step 2: Preparation of 1-{4-bromo-3-[(3,4-dimethoxyphenyl)methoxy]-5-methylphenyl}ethanone

To a stirred mixture of 1-(4-bromo-3-hydroxy-5-methylphenyl)ethanone (700 mg, 3.05 mmol, 1.00 equiv.) and 4-(bromomethyl)-1,2-dimethoxybenzene (847 mg, 3.66 mmol, 1.20 equiv.) in MeCN (20 mL) was added K₂CO₃ (1.2 g, 9.16 mmol, 3.00 equiv.) at room temperature under nitrogen atmosphere. The resulting mixture was stirred for 2 h at 60° C. under nitrogen atmosphere and then cooled to room temperature. The resulting mixture was filtered and the filter cake was washed with EtOAc (3×50 mL). The filtrate was concentrated under reduced pressure and the residue was purified by silica gel column chromatography (PE/EtOAc (0% to 30% in 30 min)) to afford 1-{4-bromo-3-[(3,4-dimethoxyphenyl)methoxy]-5-methylphenyl}ethanone (375 mg, 32.3%) as a white solid. ¹H NMR (300 MHz, DMSO-d₆) δ 7.61-7.57 (m, 1H), 7.49 (d, J=2.0 Hz, 1H), 7.13 (d, J=1.9 Hz, 1H), 7.06-6.95 (m, 2H), 5.21 (s, 2H), 3.77 (s, 6H), 2.58 (s, 3H), 2.44 (s, 3H).

### Step 3: Preparation of 2-{4-bromo-3-[(3,4-dimethoxyphenyl)methoxy]-5-methylphenyl}propan-2-ol

To a stirred mixture of 1-{4-bromo-3-[(3,4-dimethoxyphenyl)methoxy]-5-methylphenyl}ethanone (850 mg, 2.24 mmol, 1.00 equiv.) in THF (45 mL) was added bromo(methyl)magnesium (4.5 mL, 4.48 mmol, 2.00 equiv., 1 M in THF) dropwise at 0° C. under nitrogen atmosphere. The resulting mixture was stirred for 1 h at room temperature and then quenched by the addition of water (100 mL) 0° C. The resulting mixture was extracted with EtOAc (3×100 mL). The combined organic layers were washed with brine (1×100 mL), dried over anhydrous Na₂SO₄, filtered, and concentrated. The residue was purified by silica gel column chromatography (PE/EtOAc (0% to 50% gradient in 30 min) to afford 2-{4-bromo-3-[(3,4-dimethoxyphenyl)methoxy]-5-methylphenyl}propan-2-ol (650 mg, 73.3%) as a white solid. LC-MS: (ES−H, m/z) [M−H]⁻=393.0. ¹H NMR (300 MHz, DMSO-d₆) δ 7.13 (t, J=2.5 Hz, 2H), 7.06-6.93 (m, 3H), 5.10 (s, 2H), 5.06 (s, 1H), 3.76 (d, J=2.8 Hz, 6H), 2.35 (s, 3H), 1.41 (s, 6H).

### Step 4: Preparation of ethyl (S)-3-((tert-butoxycarbonyl)amino)-3-(2′-((3,4-dimethoxybenzyl)oxy)-4-fluoro-4′-(2-hydroxypropan-2-yl)-5,6′-dimethyl-[1,1′-biphenyl]-3-yl)propanoate

To a stirred mixture of 2-{4-bromo-3-[(3,4-dimethoxyphenyl)methoxy]-5-methylphenyl}propan-2-ol (500 mg, 1.26 mmol, 1.00 equiv.) and ethyl (3S)-3-[(tert-butoxycarbonyl)amino]-3-[2-fluoro-3-methyl-5-(4,4,5,5-tetramethyl-1,3,2-dioxaborolan-2-yl)phenyl]propanoate (627 mg, 1.39 mmol, 1.10 equiv.) in dioxane (10 mL) and H₂O (1 mL) were added K₂CO₃ (524 mg, 3.79 mmol, 3.00 equiv.) and Pd(dppf)Cl₂ (92 mg, 0.12 mmol, 0.10 equiv.) at room temperature under nitrogen atmosphere. The resulting mixture was stirred for 2 h at 100° C. under nitrogen atmosphere and then cooled to room temperature. The resulting mixture was filtered and the filter cake was washed with EtOAc (3×50 mL). The combined organic layers were concentrated under reduced pressure and The residue was purified by silica gel column chromatography (PE/EtOAc (0% to 50% gradient in 30 min) to afford ethyl (S)-3-((tert-butoxycarbonyl)amino)-3-(2′-((3,4-dimethoxybenzyl)oxy)-4-fluoro-4′-(2-hydroxypropan-2-yl)-5,6′-dimethyl-[1,1′-biphenyl]-3-yl)propanoate (700 mg, 86.5%) as a white solid. LC-MS: (ES−H, m/z) [M−H]⁻=640.4. ¹H NMR (300 MHz, DMSO-d₆) δ 7.50 (d, J=8.8 Hz, 1H), 7.12-6.96 (m, 4H), 6.87-6.78 (m, 3H), 5.28 (s, 1H), 5.01 (s, 1H), 4.92 (s, 2H), 4.10-3.92 (m, 2H), 3.71 (s, 3H), 3.63 (s, 3H), 2.64 (brs, 2H), 2.23 (d, J=1.9 Hz, 3H), 1.99 (s, 3H), 1.44 (s, 6H), 1.31 (s, 9H), 1.27-1.04 (m, 3H).

### Step 5: Preparation of ethyl (3S)-3-amino-3-[4-fluoro-2′-hydroxy-4′-(2-hydroxypropan-2-yl)-5,6′-dimethyl-[1,1′-biphenyl]-3-yl]propanoate

To a stirred mixture of ethyl (3S)-3-[(tert-butoxycarbonyl)amino]-3-{2′-[(3,4-dimethoxyphenyl)methoxy]-4-fluoro-4′-(2-hydroxypropan-2-yl)-5,6′-dimethyl-[1,1′-biphenyl]-3-yl}propanoate (500 mg, 0.46 mmol, 1.00 equiv.) in CH₂Cl₂ (5 mL) was added HCl (gas) in 1,4-dioxane (5 mL, 4 M) at 0° C. under nitrogen atmosphere. The resulting mixture was stirred for 1 h at 0° C. under nitrogen atmosphere. The resulting mixture was diluted with water (20 mL), neutralized to pH 7 with NH₃·H₂O, and extracted with CH₂Cl₂ (3×50 mL). The combined organic layers were dried over anhydrous Na₂SO₄, filtered, and concentrated. The residue was purified by Prep-TLC (CH₂Cl₂/MeOH=15/1) to afford ethyl (3S)-3-amino-3-[4-fluoro-2′-hydroxy-4′-(2-hydroxypropan-2-yl)-5,6′-dimethyl-[1,1′-biphenyl]-3-yl]propanoate (126 mg, 41.3%) as an off-white solid. LC-MS: (ES+H, m/z) [M+H]⁺=390.2.

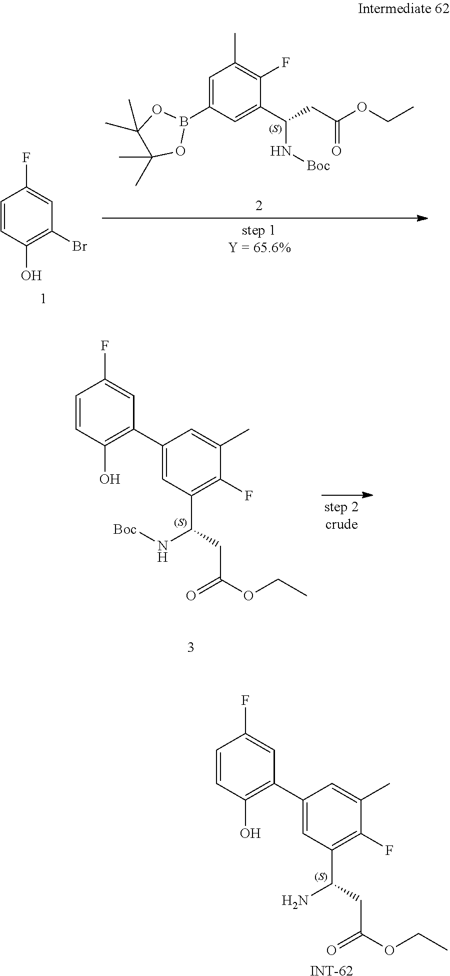

### Step 1: Preparation of ethyl (3S)-3-[(tert-butoxycarbonyl)amino]-3-{4,5′-difluoro-2′-hydroxy-5-methyl-[1,1′-biphenyl]-3-yl}propanoate

To a stirred solution of 2-bromo-4-fluorophenol (635 mg, 3.33 mmol, 1.00 equiv.) and ethyl (3S)-3-[(tert-butoxycarbonyl)amino]-3-[2-fluoro-3-methyl-5-(4,4,5,5-tetramethyl-1,3,2-dioxaborolan-2-yl)phenyl]propanoate (1.65 g, 3.66 mmol, 1.10 equiv.) in dioxane (20 mL) and H₂O (2 mL) were added K₂CO₃ (1.15 g, 8.31 mmol, 2.50 equiv.) and Pd(dppf)Cl₂ (486 mg, 0.67 mmol, 0.20 equiv.) in portions at room temperature. The resulting mixture was stirred for 2 h at 100° C. under nitrogen atmosphere and then cooled to room temperature. The reaction was quenched by the addition of ice water (40 mL) at room temperature and extracted with EtOAc (3×20 mL). The combined organic layers were washed with brine (1×20 mL), dried over anhydrous Na₂SO₄, filtered, and concentrated. The residue was purified by silica gel column chromatography (petroleum ether/EtOAc (0% to 60% gradient in 25 min) to afford ethyl (3S)-3-[(tert-butoxycarbonyl)amino]-3-{4,5′-difluoro-2′-hydroxy-5-methyl-[1,1′-biphenyl]-3-yl}propanoate (950 mg, 65.6%) as a yellow solid. LC-MS: (ES−H, m/z) [M−H]⁻=434.2.

### Step 2: Preparation of ethyl (3S)-3-amino-3-{4,5′-difluoro-2′-hydroxy-5-methyl-[1,1′-biphenyl]-3-yl}propanoate

A solution of ethyl (3S)-3-[(tert-butoxycarbonyl)amino]-3-{4,5′-difluoro-2′-hydroxy-5-methyl-[1,1′-biphenyl]-3-yl}propanoate (950 mg, 2.18 mmol, 1.00 equiv.) and HCl (gas) in 1,4-dioxane (4.5 mL, 4 M) in CH₂Cl₂ (9 mL) was stirred for 2 h at room temperature. The resulting mixture was concentrated under reduced pressure to afford ethyl (3S)-3-amino-3-{4,5′-difluoro-2′-hydroxy-5-methyl-[1,1′-biphenyl]-3-yl}propanoate (900 mg, crude) as a yellow oil. LC-MS: (ES+H, m/z) [M+H]⁺=336.0.

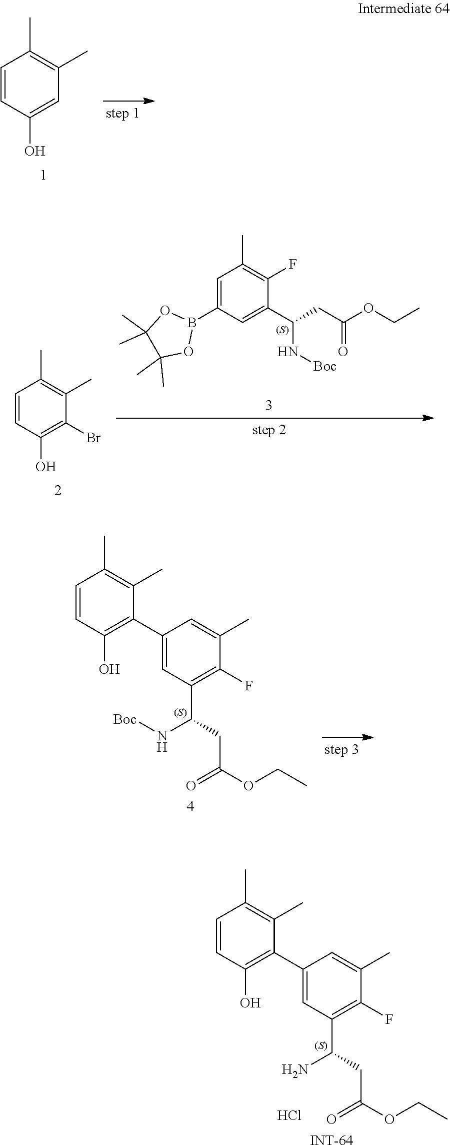

### Step 1: Preparation of 2-bromo-3,4-dimethylphenol

To a stirred mixture of 3,4-dimethylphenol (20.00 g, 163.71 mmol, 1.00 equiv.) in CH₃COOH (300 mL) was added Br₂ (8.4 mL, 163.71 mmol, 1.00 equiv.) in H₂O (35 mL) and CH₃COOH (5 mL) dropwise at 0° C. under nitrogen atmosphere. The resulting mixture was stirred for 2 h at room temperature and then was concentrated. The resulting mixture was diluted with diethyl ether (600 mL) and washed with Na₂S₂O₃ (aq. 0.63 M) (1×500 mL). The organic layer was washed with sat. NaHCO₃ (aq.) (1×500 mL), dried over anhydrous Na₂SO₄, filtered, and concentrated. The residue was purified by silica gel column chromatography (PE/CH₂Cl₂ (0%-30% in 50 min) to afford 2-bromo-3,4-dimethylphenol (1 g, crude) as a white solid. The crude product (1 g) was purified by Prep-SFC (Column: CHIRALPAK IG, 3*25 cm, 5 μm; Mobile Phase A: CO₂, Mobile Phase B: MeOH:EtOH:Hexane=1:1:2 (20 mM NH₃); Flow rate: 100 mL/min; Gradient: isocratic 10% B; Column Temperature (° C.) 35; Back Pressure (bar) 100; wavelength: 284/220 nm; Sample Solvent: MeOH:CH₂Cl₂=16:1; Injection Volume: 0.3 mL) to afford 2-bromo-3,4-dimethylphenol (725 mg, 2.2%) as a light yellow solid. LC-MS: (ES−H, m/z) [M−H]⁻=199.0. ¹H NMR (300 MHz, DMSO-d₆) δ 9.80 (s, 1H), 6.94 (d, J=8.2 Hz, 1H), 6.69 (d, J=8.2 Hz, 1H), 2.28 (s, 3H), 2.19 (s, 3H).

### Step 2: Preparation of ethyl (3S)-3-[(tert-butoxycarbonyl)amino]-3-{4-fluoro-6′-hydroxy-2′,3′,5-trimethyl-[1,1′-biphenyl]-3-yl}propanoate

To a stirred mixture of 2-bromo-3,4-dimethylphenol (650 mg, 3.23 mmol, 1.00 equiv.) and ethyl (3S)-3-[(tert-butoxycarbonyl)amino]-3-[2-fluoro-3-methyl-5-(4,4,5,5-tetramethyl-1,3,2-dioxaborolan-2-yl)phenyl]propanoate (1.46 g, 3.23 mmol, 1.00 equiv.) in dioxane (30 mL) and H₂O (1.5 mL) was added Pd(dppf)Cl₂ CH₂Cl₂ (526 mg, 0.65 mmol, 0.20 equiv.) and K₂CO₃ (1.34 g, 9.69 mmol, 3.00 equiv.) at room temperature. The resulting mixture was stirred for 3 h at 100° C. under nitrogen atmosphere and then cooled to room temperature and concentrated. The residue was purified by silica gel column chromatography (PE/EtOAc (10%-30% gradient in 30 min)) to afford ethyl (3S)-3-[(tert-butoxycarbonyl)amino]-3-{4-fluoro-6′-hydroxy-2′,3′,5-trimethyl-[1,1′-biphenyl]-3-yl}propanoate (476 mg, 33.1%) as a white solid. LC-MS: (ES−H, m/z) [M−H]⁻=444.2. ¹H NMR (300 MHz, CD₃OD) δ 6.96 (dd, J=7.8, 3.2 Hz, 3H), 6.63 (d, J=8.2 Hz, 1H), 5.37 (s, 1H), 4.17-4.08 (m, 2H), 2.79 (q, J=8.0 Hz, 2H), 2.32 (d, J=2.1 Hz, 3H), 2.21 (s, 3H), 1.96-1.86 (m, 3H), 1.42 (s, 9H), 1.27-1.21 (m, 3H). ¹⁹F NMR (282 MHz, CD₃OD) δ −128.20.

### Step 3: Preparation of ethyl (3S)-3-amino-3-{4-fluoro-6′-hydroxy-2′,3′,5-trimethyl-[1,1′-biphenyl]-3-yl}propanoate, HCl salt

A mixture of (3S)-3-[(tert-butoxycarbonyl)amino]-3-{4-fluoro-6′-hydroxy-2′,3′,5-trimethyl-[1,1′-biphenyl]-3-yl}propanoate (450 mg, 1.01 mmol, 1.00 equiv.) and HCl (gas) in 1,4-dioxane (5 mL, 20.00 mmol, 19.80 equiv., 4 M) in CH₂Cl₂ (5 mL) was stirred for 2 h at room temperature. The resulting mixture was concentrated under reduced pressure to afford ethyl (3S)-3-amino-3-{4-fluoro-6′-hydroxy-2′,3′,5-trimethyl-[1,1′-biphenyl]-3-yl}propanoate, HCl salt (400 mg, crude) as a brown oil. LC-MS: (ES+H, m/z) [M+H]⁺=346.0.

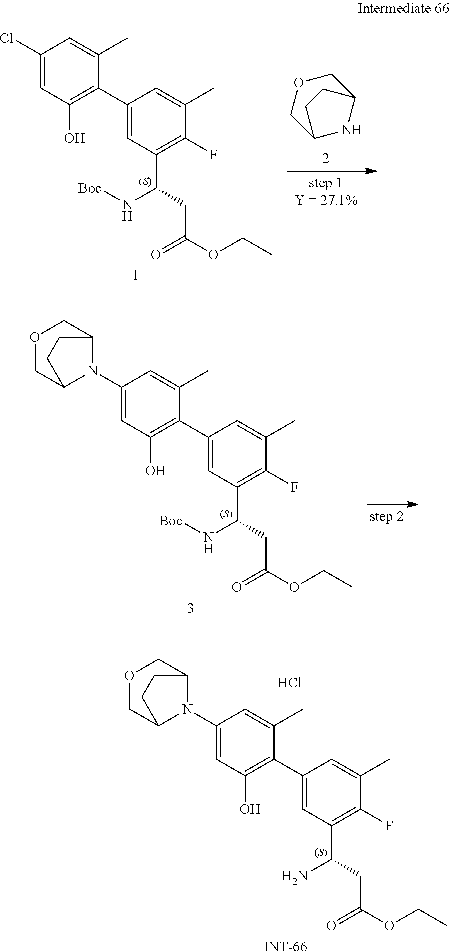

### Step 1: Preparation of ethyl (3S)-3-(4′-(3-oxa-8-azabicyclo[3.2.1]octan-8-yl)-4-fluoro-2′-hydroxy-5,6′-dimethyl-[1,1′-biphenyl]-3-yl)-3-((tert-butoxycarbonyl)amino)propanoate

To a stirred mixture of ethyl (S)-3-((tert-butoxycarbonyl)amino)-3-(4′-chloro-4-fluoro-2′-hydroxy-5,6′-dimethyl-[1,1′-biphenyl]-3-yl)propanoate (200 mg, 0.43 mmol, 1.00 equiv.) and 3-oxa-8-azabicyclo[3.2.1]octane (437 mg, 3.86 mmol, 3.00 equiv.) in toluene (20 mL) were added {1,3-bis[2,6-bis(pentan-3-yl)phenyl]-4,5-dichloro-2,3-dihydro-1H-imidazol-2-yl}dichloro(2-methyl-1lambda4-pyridin-1-yl)palladium (108 mg, 0.13 mmol, 0.10 equiv.) and Cs₂CO₃ (1259 mg, 3.86 mmol, 3.00 equiv.). The resulting mixture was stirred for 2 h at 120° C. under nitrogen atmosphere and then cooled to room temperature. The resulting mixture was concentrated under reduced pressure. The residue was purified by silica gel column chromatography (PE/EtOAc (0 to 30% gradient in 30 min)) to afford ethyl (3S)-3-(4′-(3-oxa-8-azabicyclo[3.2.1]octan-8-yl)-4-fluoro-2′-hydroxy-5,6′-dimethyl-[1,1′-biphenyl]-3-yl)-3-((tert-butoxycarbonyl)amino)propanoate (190 mg, 27.1%) as a yellow oil. LC-MS: (ES+H, m/z) [M+H]⁺=543.3. ¹H NMR (300 MHz, CD₃OD) δ 7.01 (t, J=5.5 Hz, 2H), 6.35 (s, 1H), 6.27 (s, 1H), 5.40-5.31 (m, 1H), 4.17-4.10 (m, 2H), 4.07 (s, 2H), 3.93 (d, J=10.7 Hz, 2H), 3.53 (d, J=10.8 Hz, 2H), 2.86-2.72 (m, 2H), 2.31 (s, 3H), 2.06-2.04 (m, 4H), 1.99 (s, 3H), 1.43 (s, 9H), 1.23 (t, J=7.1 Hz, 3H).

### Step 2: Preparation of ethyl (3S)-3-(4′-(3-oxa-8-azabicyclo[3.2.1]octan-8-yl)-4-fluoro-2′-hydroxy-5,6′-dimethyl-[1,1′-biphenyl]-3-yl)-3-aminopropanoate hydrochloride

To a stirred solution of ethyl (3S)-3-(4′-(3-oxa-8-azabicyclo[3.2.1]octan-8-yl)-4-fluoro-2′-hydroxy-5,6′-dimethyl-[1,1′-biphenyl]-3-yl)-3-((tert-butoxycarbonyl)amino)propanoate (180 mg, 0.33 mmol, 1.00 equiv.) in CH₂Cl₂ (3 mL) was added HCl (gas) in 1,4-dioxane (3 mL, 4 M) dropwise at room temperature under nitrogen atmosphere. The resulting mixture was stirred for 1 h at room temperature and then concentrated to provide ethyl (3S)-3-(4′-(3-oxa-8-azabicyclo[3.2.1]octan-8-yl)-4-fluoro-2′-hydroxy-5,6′-dimethyl-[1,1′-biphenyl]-3-yl)-3-aminopropanoate hydrochloride (170 mg crude) as a yellow solid. LC-MS: (ES+H, m/z) [M+H]⁺=443.2

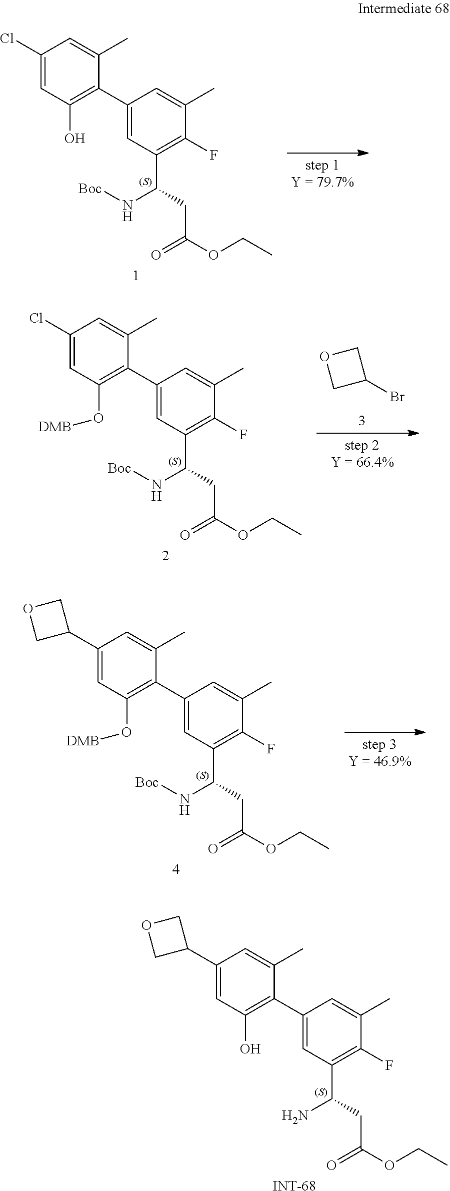

### Step 1: Preparation of ethyl (S)-3-((tert-butoxycarbonyl)amino)-3-(4′-chloro-2′-((3,4-dimethoxybenzyl)oxy)-4-fluoro-5,6′-dimethyl-[1,1′-biphenyl]-3-yl)propanoate

To a stirred mixture of ethyl (3S)-3-[(tert-butoxycarbonyl)amino]-3-{4′-chloro-4-fluoro-2′-hydroxy-5,6′-dimethyl-[1,1′-biphenyl]-3-yl}propanoate (1.00 g, 2.14 mmol, 1.00 equiv.) and K₂CO₃ (889 mg, 6.43 mmol, 3.00 equiv.) in DMF (10 mL) was added 4-(bromomethyl)-1,2-dimethoxybenzene (595 mg, 2.57 mmol, 1.20 equiv.) dropwise at room temperature under nitrogen atmosphere. The resulting mixture was stirred at 60° C. for 2 h and then cooled to room temperature and concentrated. The residue was purified by silica gel column chromatography (PE/EtOAc (0 to 30% gradient in 30 min)) to afford ethyl (S)-3-((tert-butoxycarbonyl)amino)-3-(4′-chloro-2′-((3,4-dimethoxybenzyl)oxy)-4-fluoro-5,6′-dimethyl-[1,1′-biphenyl]-3-yl)propanoate (1.00 g, 79.7%) as a white solid. LC-MS: (ES+H, m/z) [M+H]⁺=616.2. ¹H NMR (300 MHz, DMSO-d₆) δ 7.50 (s, 1H), 7.11-6.70 (m, 7H), 5.27 (s, 1H), 4.97 (s, 2H), 4.04-3.95 (m, 2H), 3.71 (s, 3H), 3.64 (s, 3H), 2.64 (s, 2H), 2.22 (s, 3H), 1.98 (d, J=3.3 Hz, 3H), 1.30 (s, 9H), 1.11 (t, J=7.1 Hz, 3H).

### Step 2: Preparation of ethyl (S)-3-((tert-butoxycarbonyl)amino)-3-(2′-((3,4-dimethoxybenzyl)oxy)-4-fluoro-5,6′-dimethyl-4′-(oxetan-3-yl)-[1,1′-biphenyl]-3-yl)propanoate

A mixture of ethyl (3S)-3-[(tert-butoxycarbonyl)amino]-3-{4′-chloro-2′-[(3,4-dimethoxyphenyl)methoxy]-4-fluoro-5,6′-dimethyl-[1,1′-biphenyl]-3-yl}propanoate (800 mg, 1.29 mmol, 1.00 equiv.), 3-bromooxetane (355 mg, 2.59 mmol, 2.00 equiv.), octan-1-ol (338 mg, 2.59 mmol, 2.00 equiv.), Zn (254 mg, 3.89 mmol, 3.00 equiv.), TMEDA (452 mg, 3.89 mmol, 3.00 equiv.), XPhos (30 mg, 0.06 mmol, 0.05 equiv.), octanoate sodium (338 mg, 2.59 mmol, 2.00 equiv.) and NaCl (227 mg, 3.89 mmol, 3.00 equiv.) in water (8 mL) was stirred overnight at 70° C. under nitrogen atmosphere. After cooling to room temperature, the reaction mixture was concentrated under reduced pressure. The residue was purified by silica gel column chromatography (PE/EtOAc (5:1) to afford ethyl (3S)-3-[(tert-butoxycarbonyl)amino]-3-{2′-[(3,4-dimethoxyphenyl)methoxy]-4-fluoro-5,6′-dimethyl-4′-(oxetan-3-yl)-[1,1′-biphenyl]-3-yl}propanoate (550 mg, 66.4%) as a off-white solid. LC-MS: (ES+H, m/z) [M+H]⁺=638.4. ¹H NMR (300 MHz, DMSO-d₆) δ 7.49 (d, J=8.8 Hz, 1H), 7.15-6.96 (m, 3H), 6.94-6.84 (m, 2H), 6.79 (d, J=10.0 Hz, 2H), 5.28 (s, 1H), 5.02-4.89 (m, 4H), 4.66 (dd, J=6.8, 5.8 Hz, 2H), 4.23 (p, J=7.6 Hz, 1H), 4.01 (ddt, J=11.3, 7.0, 3.7 Hz, 2H), 3.71 (s, 3H), 3.63 (s, 3H), 2.64 (s, 2H), 2.23 (d, J=1.8 Hz, 3H), 2.00 (d, J=1.1 Hz, 3H), 1.36-1.23 (m, 9H), 1.12 (t, J=7.1 Hz, 3H). ¹⁹F NMR (282 MHz, DMSO-d₆) δ −127.19.

### Step 3: Preparation of ethyl (S)-3-amino-3-(4-fluoro-2′-hydroxy-5,6′-dimethyl-4′-(oxetan-3-yl)-[1,1′-biphenyl]-3-yl)propanoate

A mixture of ethyl (3S)-3-[(tert-butoxycarbonyl)amino]-3-{2′-[(3,4-dimethylphenyl)methoxy]-4-fluoro-5,6′-dimethyl-4′-(oxetan-3-yl)-[1,1′-biphenyl]-3-yl}propanoate (200 mg, 0.33 mmol, 1.00 equiv.) (100 mg, 0.16 mmol, 1.00 equiv.) and TFA (1.00 mL, 13.45 mmol, 40.78 equiv.) in CH₂Cl₂ (2 mL) was stirred for 2 h at room temperature under nitrogen atmosphere. The mixture was basified to pH 9 with saturated aq. NaHCO₃ and then concentrated under reduced pressure. The residue was purified by Prep-TLC (CH₂Cl₂/MeOH 10:1) to afford ethyl (S)-3-amino-3-(4-fluoro-2′-hydroxy-5,6′-dimethyl-4′-(oxetan-3-yl)-[1,1′-biphenyl]-3-yl)propanoate (60 mg, 46.9%) as an off-white solid. LC-MS: (ES+H, m/z) [M+H]⁺=388.2.

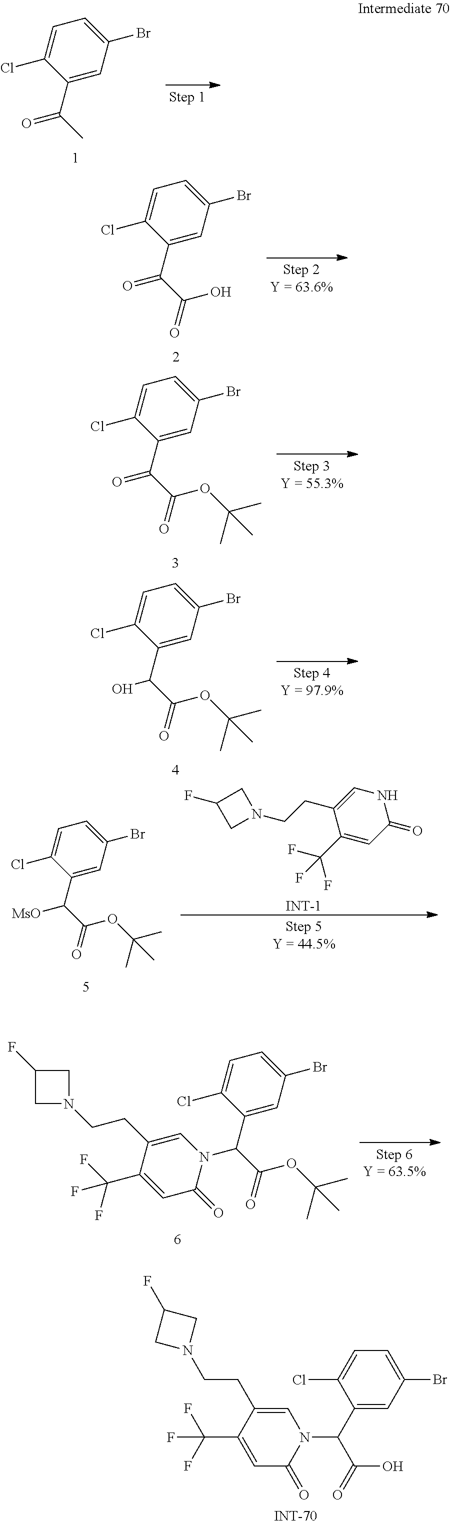

### Step 1: Preparation of (5-bromo-2-chlorophenyl)(oxo)acetic acid

To a stirred solution of 1-(5-bromo-2-chlorophenyl)ethanone (5.00 g, 21.41 mmol, 1.00 equiv.) in pyridine (20 mL) was added SeO₂ (3.56 g, 32.12 mmol, 1.50 equiv.) in portions at room temperature. The resulting mixture was stirred for 2 h at 100° C. under nitrogen atmosphere and then cooled to room temperature and diluted with 1 N HCl. (aq. 100 mL). The resulting mixture was extracted with EtOAc (3×100 mL) and the combined organic layers were concentrated to provide (5-bromo-2-chlorophenyl)(oxo)acetic acid (3.5 g, crude) as a colorless oil. LC-MS: (ES−H, m/z) [M−H]⁻=262.8. ¹H NMR (300 MHz, DMSO-d₆) δ 7.93 (d, J=2.4 Hz, 1H), 7.85 (dd, J=8.6, 2.5 Hz, 1H), 7.58 (d, J=8.6 Hz, 1H).

### Step 2: Preparation of tert-butyl 2-(5-bromo-2-chlorophenyl)-2-oxoacetate

To a stirred solution of (5-bromo-2-chlorophenyl)(oxo)acetic acid (3.50 g, 13.28 mmol, 1.00 equiv.) in DCM (40 mL) was added N,N′-diisopropyltert-butoxymethanimidamide (5.32 g, 26.56 mmol, 2.00 equiv.) in portions at room temperature. The resulting mixture was stirred overnight at room temperature and then diluted with water (100 mL) and extracted with CH₂Cl₂ (3×100 mL). The combined organic layers were washed with brine (1×100 mL), dried over anhydrous Na₂SO₄, filtered, and concentrated. The residue was purified by silica gel column chromatography (PE/EtOAc (0%-50%) gradient in 30 min to afford tert-butyl 2-(5-bromo-2-chlorophenyl)-2-oxoacetate (3 g, 63.6%) as a colorless oil. LC-MS: (ES+H, m/z) [M+H]⁺=319.2. ¹H NMR (400 MHz, CDCl₃) δ 7.83 (d, J=2.4 Hz, 1H), 7.61 (dd, J=8.5, 2.4 Hz, 1H), 7.30 (d, J=8.5 Hz, 1H), 1.59 (s, 9H).

### Step 3: Preparation of tert-butyl 2-(5-bromo-2-chlorophenyl)-2-hydroxyacetate

To a stirred solution of tert-butyl 2-(5-bromo-2-chlorophenyl)-2-oxoacetate (2.90 g, 9.07 mmol, 1.00 equiv.) in EtOH (30 mL) and HOAc (3 mL) was added NaBH₃CN (0.63 g, 9.98 mmol, 1.10 equiv.) in portions at room temperature. The resulting mixture was stirred for 1 h at room temperature and then diluted with EtOAc (150 mL) and washed with water (2×100 mL). The combined organic layers were washed with brine (1×100 mL), dried over anhydrous Na₂SO₄, filtered, and concentrated. The residue was purified by silica gel column chromatography (petroleum ether/EtOAc (0%-50% gradient in 30 min)) to afford tert-butyl 2-(5-bromo-2-chlorophenyl)-2-hydroxyacetate (1.7 g, 55.3%) as a colorless oil. LC-MS: (ES−H, m/z) [M−H]⁻=321.0. ¹H NMR (400 MHz, CDCl₃) δ 7.54 (d, J=2.4 Hz, 1H), 7.37 (dd, J=8.5, 2.4 Hz, 1H), 7.24 (d, J=8.5, 1H), 5.38 (s, 1H), 1.42 (s, 9H).

### Step 4: Preparation of tert-butyl 2-(5-bromo-2-chlorophenyl)-2-(methanesulfonyloxy)acetate

To a stirred solution of tert-butyl 2-(5-bromo-2-chlorophenyl)-2-hydroxyacetate (1.70 g, 5.28 mmol, 1.00 equiv.) and Et₃N (1.60 g, 15.85 mmol, 3.00 equiv.) in CH₂Cl₂ (20 mL) were added MsCl (1.82 g, 7.93 mmol, 1.50 equiv., 50% in CH₂Cl₂) dropwise at room temperature. The resulting mixture was stirred for 3 h at room temperature under nitrogen atmosphere and then diluted with water (100 mL) and extracted with CH₂Cl₂ (1×80 mL). The combined organic layers were washed with brine (1×100 mL), dried over anhydrous Na₂SO₄, filtered, and concentrated. The residue was purified by silica gel column chromatography (petroleum ether/EtOAc (0%-30% gradient in 30 min)) to afford tert-butyl 2-(5-bromo-2-chlorophenyl)-2-(methanesulfonyloxy)acetate (2.3 g, 97.9%) as a colorless oil. ¹H NMR (400 MHz, CDCl₃) δ 7.53 (d, J=2.4 Hz, 1H), 7.40 (dd, J=8.6, 2.4 Hz, 1H), 7.24 (d, J=8.6 Hz, 1H), 6.13 (s, 1H), 3.13 (s, 3H), 1.38 (s, 9H).

### Step 5: Preparation of tert-butyl 2-(5-bromo-2-chlorophenyl)-2-{5-[2-(3-fluoroazetidin-1-yl)ethyl]-2-oxo-4-(trifluoromethyl)pyridin-1-yl}acetate

To a stirred solution of tert-butyl 2-(5-bromo-2-chlorophenyl)-2-(methanesulfonyloxy)acetate (1.90 g, 4.75 mmol, 1.15 equiv.) in MeCN (20 mL) were added 5-[2-(3-fluoroazetidin-1-yl)ethyl]-4-(trifluoromethyl)-1H-pyridin-2-one (1.09 g, 4.13 mmol, 1.00 equiv.) and K₂CO₃ (1.71 g, 12.40 mmol, 3.00 equiv.) in portions at room temperature. The resulting mixture was stirred for 3 h at 80° C. under nitrogen atmosphere and then cooled to room temperature. The resulting mixture was filtered and the filter cake was washed with EtOAc (3×100 mL). The combined filtrate was concentrated under reduced pressure and the residue was purified by reversed-phase flash chromatography (C18 gel; mobile phase, MeCN in water (0.1% NH₃·H₂O), 0% to 90% gradient in 50 min; detector, UV 254 nm) to provide tert-butyl 2-(5-bromo-2-chlorophenyl)-2-{5-[2-(3-fluoroazetidin-1-yl)ethyl]-2-oxo-4-(trifluoromethyl)pyridin-1-yl}acetate (1.1 g, 44.5%) as a white solid. LC-MS: (ES+H, m/z) [M+H]⁺=567.1. 1H NMR (300 MHz, CD₃OD) δ 7.71 (dd, J=8.5, 2.4 Hz, 1H), 7.65 (d, J=2.3 Hz, 1H), 7.52 (d, J=8.5 Hz, 1H), 7.39 (s, 1H), 6.90 (s, 1H), 6.60 (s, 1H), 5.14-4.87 (m, 1H), 3.54-3.38 (m, 2H), 3.17-2.98 (m, 2H), 2.64-2.46 (m, 4H), 1.52 (s, 9H).

### Step 6: Preparation of (5-bromo-2-chlorophenyl)({5-[2-(3-fluoroazetidin-1-yl)ethyl]-2-oxo-4-(trifluoromethyl)pyridin-1-yl})acetic acid

To a stirred solution of tert-butyl 2-(5-bromo-2-chlorophenyl)-2-{5-[2-(3-fluoroazetidin-1-yl)ethyl]-2-oxo-4-(trifluoromethyl)pyridin-1-yl}acetate (1.10 g, 1.93 mmol, 1.00 equiv.) in CH₂C₂ (10 mL) were added CF₃COOH (10 mL) dropwise at room temperature. The resulting mixture was stirred for 2 h at room temperature and then concentrated. The residue was purified by reversed-phase flash chromatography (C18 gel; mobile phase, MeCN in water (0.1% NH₃·H₂O), 0% to 50% gradient in 30 min; detector, UV 254 nm) to provide (5-bromo-2-chlorophenyl)({5-[2-(3-fluoroazetidin-1-yl)ethyl]-2-oxo-4-(trifluoromethyl)pyridin-1-yl})acetic acid (700 mg, 63.5%) as a yellow oil. LC-MS: (ES+H, m/z) [M+H]⁺=511.0, 513.0. ¹H NMR (300 MHz, DMSO-d₆) δ 7.65 (d, J=7.7 Hz, 2H), 7.54-7.42 (m, 2H), 6.78 (s, 1H), 6.45 (s, 1H), 5.76 (s, 1H), 5.18-4.92 (m, 1H), 3.60 (d, J=17.2 Hz, 2H), 3.23 (d, J=24.1 Hz, 2H), 2.75-2.55 (m, 2H), 2.46 (d, J=7.1 Hz, 2H).

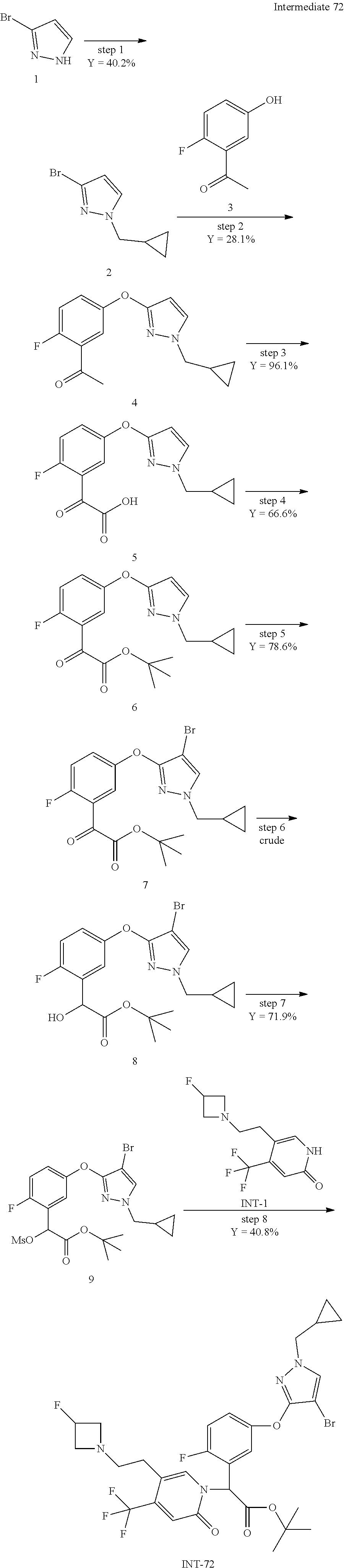

### Step 1: Preparation of 3-bromo-1-(cyclopropylmethyl)pyrazole

A mixture of 3-bromo-1H-pyrazole (10.00 g, 68.03 mmol, 1.00 equiv.), (bromomethyl)cyclopropane (11.02 g, 81.64 mmol, 1.20 equiv.) and K₂CO₃ (18.81 g, 136.07 mmol, 2.00 equiv.) in DMF (60 mL) was stirred overnight at 50° C. under nitrogen atmosphere. The resulting mixture was diluted with water (600 mL) and extracted with EtOAc (3×600 mL). The combined organic layers were washed with brine (3×600 mL), dried over anhydrous Na₂SO₄, filtered, and concentrated to provide 3-bromo-1-(cyclopropylmethyl)pyrazole (5.5 g, 40.2%) as a light yellow oil. LC-MS: (ES+H, m/z) [M+H]⁺=200.9. ¹H NMR (300 MHz, DMSO-d₆) δ 7.78 (d, J=2.3 Hz, 1H), 6.36 (d, J=2.3 Hz, 1H), 3.94 (d, J=7.2 Hz, 2H), 1.31-1.12 (m, 1H), 0.60-0.41 (m, 2H), 0.45-0.29 (m, 2H).

### Step 2: Preparation of 1-(5-{[1-(cyclopropylmethyl)pyrazol-3-yl]oxy}-2-fluorophenyl)ethanone

A mixture of 3-bromo-1-(cyclopropylmethyl)pyrazole (4.70 g, 23.35 mmol, 1.20 equiv.), 1-(2-fluoro-5-hydroxyphenyl)ethanone (3.00 g, 19.46 mmol, 1.00 equiv.), CuI (1.85 g, 9.73 mmol, 0.50 equiv.), 2-(dimethylamino)acetic acid (2.00 g, 19.46 mmol, 1.00 equiv.) and Cs₂CO₃ (12.68 g, 38.92 mmol, 2.00 equiv.) in dioxane (30 mL) was stirred overnight at 100° C. under nitrogen atmosphere and then concentrated. The residue was purified by silica gel column chromatography (PE/EtOAc (0 to 10% gradient in 30 min)) to afford 1-(5-{[1-(cyclopropylmethyl)pyrazol-3-yl]oxy}-2-fluorophenyl)ethanone (1.5 g, 28.1%) as a white solid. LC-MS: (ES+H, m/z) [M+H]⁺=275.0.

### Step 3: Preparation of (5-{[1-(cyclopropylmethyl)pyrazol-3-yl]oxy}-2-fluorophenyl)(oxo)acetic acid

A mixture of 1-(5-{[1-(cyclopropylmethyl)pyrazol-3-yl]oxy}-2-fluorophenyl)ethanone (1.50 g, 5.46 mmol, 1.00 equiv.) and SeO₂ (1.21 g, 10.93 mmol, 2.00 equiv.) in pyridine (18 mL) was stirred for 2 h at 100° C. under nitrogen atmosphere and then cooled to room temperature. The resulting mixture was diluted with 1 M HCl (aq.) (250 mL) and extracted with EtOAc (3×250 mL). The combined organic layers were washed with brine (3×250 mL), dried over anhydrous Na₂SO₄, filtered, and concentrated to provide (5-{[1-(cyclopropylmethyl)pyrazol-3-yl]oxy}-2-fluorophenyl)(oxo)acetic acid (1.6 g, 96.1%) as a white solid. LC-MS: (ES+H, m/z) [M+H]⁺=305.0.

### Step 4: Preparation of tert-butyl 2-(5-{[1-(cyclopropylmethyl)pyrazol-3-yl]oxy}-2-fluorophenyl)-2-oxoacetate

To a stirred solution of (5-{[1-(cyclopropylmethyl)pyrazol-3-yl]oxy}-2-fluorophenyl)(oxo)acetic acid (1.90 g, 6.24 mmol, 1.00 equiv.) in THF (20 mL) was added N,N′-diisopropyltert-butoxymethanimidamide (4.38 g, 21.85 mmol, 3.50 equiv.) dropwise at 0° C. under nitrogen atmosphere. The resulting mixture was stirred overnight at room temperature and then concentrated. The residue was purified by silica gel column chromatography (PE/EtOAc (0 to 20% gradient in 30 min)) to afford tert-butyl 2-(5-{[1-(cyclopropylmethyl)pyrazol-3-yl]oxy}-2-fluorophenyl)-2-oxoacetate (1.5 g, 66.6%) as a light yellow oil. LC-MS: (ES+H, m/z) [M+H]⁺=361.1.

### Step 5: Preparation of tert-butyl 2-(5-{[4-bromo-1-(cyclopropylmethyl)pyrazol-3-yl]oxy}-2-fluorophenyl)-2-oxoacetate

To a stirred solution of tert-butyl 2-(5-{[1-(cyclopropylmethyl)pyrazol-3-yl]oxy}-2-fluorophenyl)-2-oxoacetate (1.50 g, 4.16 mmol, 1.00 equiv.) in MeCN (15 mg) was added NBS (0.81 g, 4.57 mmol, 1.10 equiv.) in portions at room temperature. The resulting mixture was stirred for 1 h at 40° C. under nitrogen atmosphere and then cooled to room temperature and diluted with EtOAc (100 mL) and washed with water (3×80 mL). The organic layer was washed with brine (1×80 mL), dried over anhydrous Na₂SO₄, filtered, and concentrated. The residue was purified by silica gel column chromatography (PE/EtOAc (0 to 20% gradient in 30 min)) to afford tert-butyl 2-(5-{[4-bromo-1-(cyclopropylmethyl)pyrazol-3-yl]oxy}-2-fluorophenyl)-2-oxoacetate (1.6 g, 78.7%) as a yellow oil. LC-MS: (ES+H, m/z) [M+H]⁺=439.0.

### Step 6: Preparation of tert-butyl 2-(5-{[4-bromo-1-(cyclopropylmethyl)pyrazol-3-yl]oxy}-2-fluorophenyl)-2-hydroxyacetate

To a stirred solution of tert-butyl 2-(5-{[4-bromo-1-(cyclopropylmethyl)pyrazol-3-yl]oxy}-2-fluorophenyl)-2-oxoacetate (1.60 g, 3.64 mmol, 1.00 equiv.) in EtOH (18 mL) were added NaBH₃CN (0.27 g, 4.37 mmol, 1.20 equiv.) and HOAc (1.8 mL) in portions at room temperature. The resulting mixture was stirred for 1 h at room temperature and then diluted with EtOAc (100 mL) and washed with water (2×80 mL). The organic layer was washed with brine, dried over anhydrous Na₂SO₄, filtered, and concentrated to provide tert-butyl 2-(5-{[4-bromo-1-(cyclopropylmethyl)pyrazol-3-yl]oxy}-2-fluorophenyl)-2-hydroxyacetate (1.7 g, crude) as a yellow oil. LC-MS: (ES−H, m/z) [M−H]⁻=439.0. ¹H NMR (400 MHz, CDCl₃) δ7.48 (s, 1H), 7.10 (dd, J=5.8, 2.9 Hz, 1H), 7.04-7.02 (m, 1H), 6.99 (d, J=8.9 Hz, 1H), 5.23 (s, 1H), 3.85 (d, J=7.2 Hz, 2H), 1.41 (s, 9H), 1.28-1.21 (m, 1H), 0.72-0.62 (m, 2H), 0.38-0.35 (m, 2H).

### Step 7: Preparation of tert-butyl 2-(5-{[4-bromo-1-(cyclopropylmethyl)pyrazol-3-yl]oxy}-2-fluorophenyl)-2-(methanesulfonyloxy)acetate

To a stirred solution of tert-butyl 2-(5-{[4-bromo-1-(cyclopropylmethyl)pyrazol-3-yl]oxy}-2-fluorophenyl)-2-hydroxyacetate (1.7 g, 3.85 mmol, 1.00 equiv.) in CH₂Cl₂ (20 mL) was added MsCl (1.32 g, 5.77 mmol, 1.50 equiv., 50% in CH₂Cl₂) and Et₃N (1.17 g, 11.55 mmol, 3.00 equiv.) dropwise at room temperature. The resulting mixture was stirred for 3 h at room temperature under nitrogen atmosphere and then diluted with water (100 mL) and extracted with CH₂Cl₂ (3×80 mL). The combined organic layers were washed with brine (1×100 mL), dried over anhydrous Na₂SO₄, filtered, and concentrated. The residue was purified by silica gel column chromatography (PE/EtOAc (0 to 20% gradient in 30 min)) to afford tert-butyl 2-(5-{[4-bromo-1-(cyclopropylmethyl)pyrazol-3-yl]oxy}-2-fluorophenyl)-2-(methanesulfonyloxy)acetate (1.6 g, 71.9%) as a colorless oil. LC-MS: (ES+H, m/z) [M+H]⁺=519.0. ¹H NMR (300 MHz, CDCl₃) δ7.49 (s, 1H), 7.18-7.11 (m, 2H), 7.11-7.03 (m, 1H), 6.06 (s, 1H), 3.85 (d, J=7.2 Hz, 2H), 3.12 (s, 3H), 1.43 (s, 9H), 1.28-1.24 (m, 1H), 0.72-0.65 (m, 2H), 0.38 (t, J=5.2 Hz, 2H).

### Step 8: Preparation of tert-butyl 2-(5-{[4-bromo-1-(cyclopropylmethyl)pyrazol-3-yl]oxy}-2-fluorophenyl)-2-{5-[2-(3-fluoroazetidin-1-yl)ethyl]-2-oxo-4-(trifluoromethyl)pyridin-1-yl}acetate

To a stirred solution of tert-butyl 2-(5-{[4-bromo-1-(cyclopropylmethyl)pyrazol-3-yl]oxy}-2-fluorophenyl)-2-(methanesulfonyloxy)acetate (1.60 g, 3.08 mmol, 1.20 equiv.) in MeCN (15 mL) were added 5-[2-(3-fluoroazetidin-1-yl)ethyl]-4-(trifluoromethyl)-1H-pyridin-2-one (0.68 g, 2.56 mmol, 1.00 equiv.) and K₂CO₃ (1.06 g, 7.70 mmol, 3.00 equiv.) in portions at room temperature. The resulting mixture was stirred for 3 h at 80° C. under nitrogen atmosphere and then cooled to room temperature. The resulting mixture was filtered and the filter cake was washed with EtOAc (3×100 mL). The filtrate was concentrated under reduced pressure and the residue was purified by reversed-phase flash chromatography (C18 gel; mobile phase, MeCN in water (0.11% NH₃·H₂O), 0% to 100% gradient in 40 min; detector, UV 254 nm) to provide tert-butyl 2-(5-{[4-bromo-1-(cyclopropylmethyl)pyrazol-3-yl]oxy}-2-fluorophenyl)-2-{5-[2-(3-fluoroazetidin-1-yl)ethyl]-2-oxo-4-(trifluoromethyl)pyridin-1-yl}acetate (800 mg, 40.8%) as a yellow oil. LC-MS: (ES+H, m/z) [M+H]⁺=689.1. ¹H NMR (400 MHz, CD₃OD) δ7.82 (s, 1H), 7.38 (s, 1H), 7.31-7.28 (m, 2H), 7.09-7.06 (m, 1H), 6.88 (s, 1H), 5.13-4.91 (m, 1H), 3.89 (d, J=7.2 Hz, 2H), 3.57-3.42 (m, 2H), 3.14-3.01 (m, 2H), 2.62-2.53 (m, 2H), 2.52-2.47 (m, 2H), 1.44 (s, 9H), 1.31-1.26 (m, 1H), 0.65-0.59 (m, 2H), 0.40-0.36 (m, 2H).

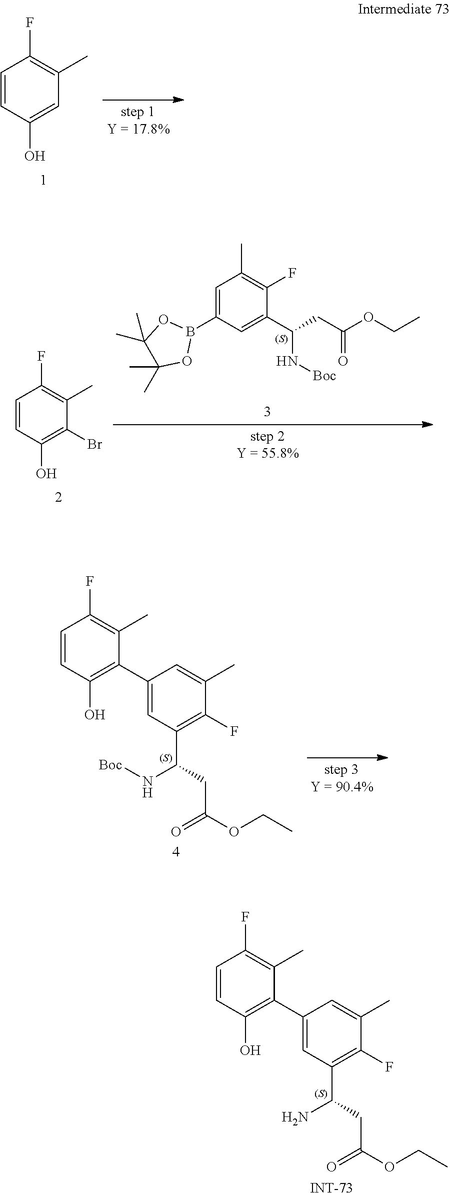

### Step 1: Preparation of 2-bromo-4-fluoro-3-methylphenol

To a stirred mixture of 4-fluoro-3-methylphenol (5.00 g 39.64 mmol, 1.00 equiv.) in AcOH (100 mL) was added Br₂ (6.34 g, 39.64 mmol, 1.00 equiv.) dropwise at 0° C. The resulting mixture was stirred for 2 h at room temperature and then was concentrated. The resulting mixture was diluted with water (100 mL) and extracted with EtOAc (3×100 mL). The combined organic layers were washed with brine (2×100 mL), dried over anhydrous Na₂SO₄, filtered, and concentrated. The residue was purified by reversed-phase flash chromatography (C18 gel; mobile phase, MeCN in water (0.1% HCOOH), 10% to 50% gradient in 10 min; detector, UV 254 nm) to afford 2-bromo-4-fluoro-3-methylphenol (5.00 g, crude). The semi-pure product (5.00 g, crude) was further purified by Prep-SFC with the following conditions (Column: NB_CHIRALPAK AD, 3*25 cm, 5 μm; Mobile Phase A: CO₂, Mobile Phase B: MeOH:EtOH:Hex=1:1:2 (20 mM NNH₃); Flow rate: 100 mL/min; Gradient: isocratic 10% B; Column Temperature (° C.) 35; Back Pressure (bar) 120; wavelength: 284/260 nm; Sample Solvent: MeOH-HPLC) to provide 2-bromo-4-fluoro-3-methylphenol (1.20 g, 17.8%) as a yellow solid. LC-MS: (ES−H, m/z) [M−H]⁻=202.90. ¹H NMR (400 MHz, DMSO-d₆) δ 10.08 (s, 1H), 7.02 (t, J=9.1 Hz, 1H), 6.81 (dd, J=9.0, 5.1 Hz, 1H), 2.25 (d, J=2.5 Hz, 3H).

### Step 2: Preparation of ethyl (3S)-3-[(tert-butoxycarbonyl)amino]-3-{3′,4-difluoro-6′-hydroxy-2′,5-dimethyl-[1,1′-biphenyl]-3-yl}propanoate

To a stirred mixture of 2-bromo-4-fluoro-3-methylphenol (450 mg, 2.19 mmol, 1.00 equiv.) and ethyl (3S)-3-[(tert-butoxycarbonyl)amino]-3-[2-fluoro-3-methyl-5-(4,4,5,5-tetramethyl-1,3,2-dioxaborolan-2-yl)phenyl]propanoate (1090 mg, 2.41 mmol, 1.10 equiv.) in dioxane (20 mL) and H₂O (1 mL) were added K₂CO₃ (606 mg, 4.39 mmol, 2.00 equiv.), Pd(dppf)Cl₂ (80 mg, 0.11 mmol, 0.05 equiv.) in portions at room temperature. The resulting mixture was stirred overnight at 100° C. under nitrogen atmosphere and then cooled to room temperature, diluted with water, and extracted with EtOAc (3×50 mL). The combined organic layers were washed with water (2×10 mL), dried over anhydrous Na₂SO₄, filtered, and concentrated. The residue was purified by reversed-phase flash chromatography (C18 gel; mobile phase, MeCN in water (0.1% HCOOH), 0% to 100% gradient in 20 min; detector, UV 220 nm) to afford ethyl (3S)-3-[(tert-butoxycarbonyl)amino]-3-{3′,4-difluoro-6′-hydroxy-2′,5-dimethyl-[1,1′-biphenyl]-3-yl}propanoate (550 mg, 55.7%) as a yellow solid. LC-MS: (ES−H, m/z) [M−H]⁻=448.15. ¹H NMR (400 MHz, DMSO-d₆) δ 9.11 (s, 1H), 7.49 (d, J=8.9 Hz, 1H), 7.04 (s, 1H), 7.02-6.91 (m, 2H), 6.73 (dd, J=8.9, 4.8 Hz, 1H), 5.28 (s, 1H), 4.13-3.93 (m, 2H), 2.66 (d, J=7.2 Hz, 2H), 2.24 (d, J=2.0 Hz, 3H), 2.03 (d, J=34.0 Hz, 1H), 1.88-1.83 (m, 3H), 1.34 (s, 8H), 1.13 (t, J=7.1 Hz, 3H).

### Step 3: Preparation of ethyl (3S)-3-amino-3-{3′,4-difluoro-6′-hydroxy-2′,5-dimethyl-[1,1′-biphenyl]-3-yl}propanoate

To a stirred mixture of ethyl (3S)-3-[(tert-butoxycarbonyl)amino]-3-{3′,4-difluoro-6′-hydroxy-2′,5-dimethyl-[1,1′-biphenyl]-3-yl}propanoate (500 mg, 1.11 mmol, 1.00 equiv.) in CH₂C₂ (10 mL) was added HCl (gas) in 1,4-dioxane (5 mL, 4 M) dropwise at 0° C. The resulting mixture was stirred for 2 h at room temperature and then concentrated to afford ethyl (3S)-3-amino-3-{3′,4-difluoro-6′-hydroxy-2′,5-dimethyl-[1,1′-biphenyl]-3-yl}propanoate (370 mg, crude) as a white solid. LC-MS: (ES+H, m/z) [M+H]⁺=350.15.

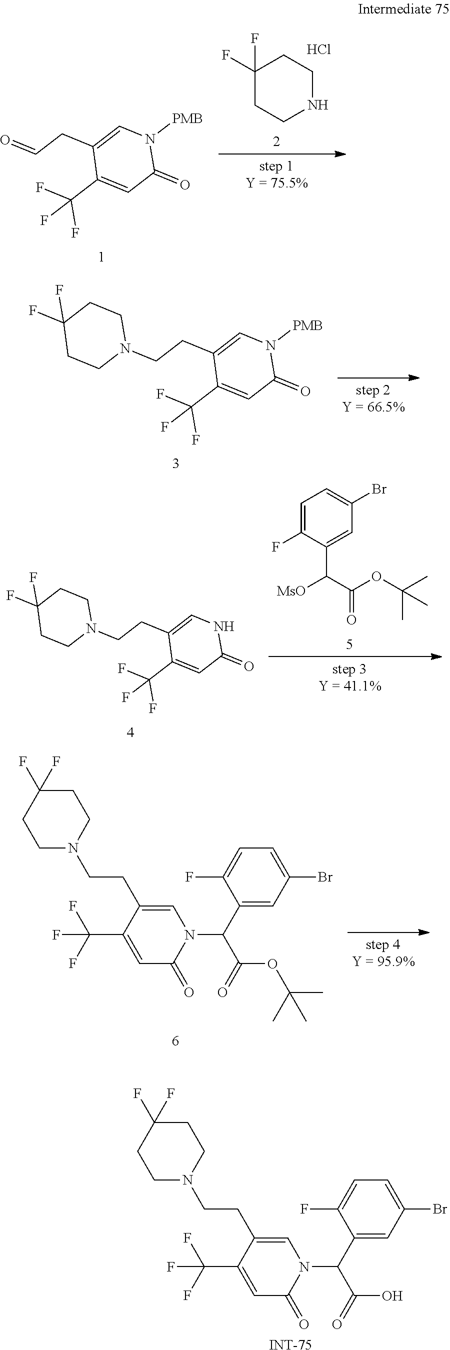

### Step 1: Preparation of 5-[2-(4,4-difluoropiperidin-1-yl)ethyl]-1-[(4-methoxyphenyl)methyl]-4-(trifluoromethyl)pyridin-2-one

To a stirred mixture of 2-{1-[(4-methoxyphenyl)methyl]-6-oxo-4-(trifluoromethyl)pyridin-3-yl}acetaldehyde (2.50 g, 7.68 mmol, 1.00 equiv.) and 4,4-difluoropiperidine (930 mg, 7.68 mmol, 1.00 equiv.) in EtOH (20 mL) were added CH₃COOH (46 mg, 0.76 mmol, 0.10 equiv.) at room temperature under nitrogen atmosphere. The resulting mixture was stirred for 1 h at room temperature under nitrogen atmosphere. To the above mixture was added NaBH₃CN (965 mg, 15.37 mmol, 2.00 equiv.) in portions over 5 min at room temperature. The resulting mixture was stirred for an additional 2 h at room temperature. The reaction was quenched by the addition of water/ice (50 mL) at room temperature and extracted with EtOAc (2×100 mL). The combined organic layers were washed with brine (2×50 mL), dried over anhydrous Na₂SO₄, filtered, and concentrated. The residue was purified by silica gel column chromatography (CH₂Cl₂/MeOH (0˜5%, 20 min) to afford 5-[2-(4,4-difluoropiperidin-1-yl)ethyl]-1-[(4-methoxyphenyl)methyl]-4-(trifluoromethyl)pyridin-2-one (2.5 g, 75.5%) as a yellow oil. LC-MS: (ES+H, m/z) [M+H]⁺=431.1. 1H NMR (400 MHz, CDCl₃) δ 7.35 (s, 1H), 7.29-7.24 (m, 2H), 6.94-6.85 (m, 3H), 5.07 (s, 2H), 3.79 (s, 3H), 2.67-2.45 (m, 7H), 1.97-1.78 (m, 4H).

### Step 2: Preparation of 5-[2-(4,4-difluoropiperidin-1-yl)ethyl]-4-(trifluoromethyl)-1H-pyridin-2-one

A mixture of 5-[2-(4,4-difluoropiperidin-1-yl)ethyl]-1-[(4-methoxyphenyl)methyl]-4-(trifluoromethyl)pyridin-2-one (2.50 g, 5.80 mmol, 1.00 equiv.) in CF₃COOH (12 mL) was stirred overnight at 80° C. under nitrogen atmosphere and then cooled to room temperature and concentrated. The residue was purified by reversed-phase flash chromatography (C18 gel; mobile phase, MeCN in water (10 mmol/L NH₄HCO₃), 10% to 50% gradient in 20 min; detector, UV 254/220 nm) to afford 5-[2-(4,4-difluoropiperidin-1-yl)ethyl]-4-(trifluoromethyl)-1H-pyridin-2-one (1.2 g, 66.5%) as a yellow oil. LC-MS: (ES+H, m/z) [M+H]⁺=311.1. ¹H NMR (400 MHz, DMSO-d₆) δ 12.28-11.94 (m, 1H), 7.55 (s, 1H), 6.68 (s, 1H), 3.17 (d, J=5.1 Hz, 4H), 2.62-2.54 (m, 4H), 1.95 (td, J=14.7, 13.5, 6.5 Hz, 4H).

### Step 3: Preparation of tert-butyl 2-(5-bromo-2-fluorophenyl)-2-{5-[2-(4,4-difluoropiperidin-1-yl)ethyl]-2-oxo-4-(trifluoromethyl)pyridin-1-yl}acetate

To a stirred mixture of 5-[2-(4,4-difluoropiperidin-1-yl)ethyl]-4-(trifluoromethyl)-1H-pyridin-2-one (1.20 g, 3.86 mmol, 1.00 equiv.) and tert-butyl 2-(5-bromo-2-fluorophenyl)-2-(methanesulfonyloxy)acetate (1.48 g, 3.86 mmol, 1.00 equiv.) in MeCN (10 mL) was added K₂CO₃ (1.07 g, 7.73 mmol, 2.00 equiv.) at room temperature under nitrogen atmosphere. The resulting mixture was stirred for 2 h at room temperature and then diluted with water (50 mL) and extracted with EtOAc (3×100 mL). The combined organic layers were washed with brine (2×100 mL), dried over anhydrous Na₂SO₄, filtered, and concentrated. The residue was purified by reversed-phase flash chromatography (C18 gel; mobile phase, MeCN in water (10 mmol/L NH₄HCO₃), 50% to 70% gradient in 20 min; detector, UV 254/220 nm) to afford tert-butyl 2-(5-bromo-2-fluorophenyl)-2-{5-[2-(4,4-difluoropiperidin-1-yl)ethyl]-2-oxo-4-(trifluoromethyl)pyridin-1-yl}acetate (950 mg, 41.1%) as colorless oil. LC-MS: (ES+H, m/z) [M+H]⁺=597.0. ¹H NMR (400 MHz, DMSO-d₆) δ 7.75-7.68 (m, 1H), 7.66 (s, 1H), 7.63 (dd, J=6.5, 2.6 Hz, 1H), 7.34 (t, J=10.0, 8.8 Hz, 1H), 6.89 (s, 1H), 6.57 (s, 1H), 2.58 (t, J=6.8 Hz, 2H), 2.46 (q, J=6.2, 5.8 Hz, 6H), 1.90-1.76 (m, 4H), 1.42 (s, 9H). ¹⁹F NMR (377 MHz, DMSO-d₆) δ −62.75, −96.06, −116.59.

### Step 4: Preparation of (5-bromo-2-fluorophenyl)({5-[2-(4,4-difluoropiperidin-1-yl)ethyl]-2-oxo-4-(trifluoromethyl)pyridin-1-yl})acetic acid

To a stirred mixture of tert-butyl 2-(5-bromo-2-fluorophenyl)-2-{5-[2-(4,4-difluoropiperidin-1-yl)ethyl]-2-oxo-4-(trifluoromethyl)pyridin-1-yl}acetate (920 mg, 1.54 mmol, 1.00 equiv.) in CH₂Cl₂ (5 mL) was added CF₃COOH (5 mL) at room temperature under nitrogen atmosphere. The resulting mixture was stirred for 1 h at room temperature and then concentrated. The residue was purified by reversed-phase flash chromatography (C18 gel; mobile phase, MeCN in water (0.1% NH₃·H₂O), 10% to 50% gradient in 10 min; detector, UV 254/220 nm) to afford (5-bromo-2-fluorophenyl)({5-[2-(4,4-difluoropiperidin-1-yl)ethyl]-2-oxo-4-(trifluoromethyl)pyridin-1-yl})acetic acid (800 mg, 95.9%) as a colorless solid. LC-MS: (ES+H, m/z) [M+H]⁺=541.0.

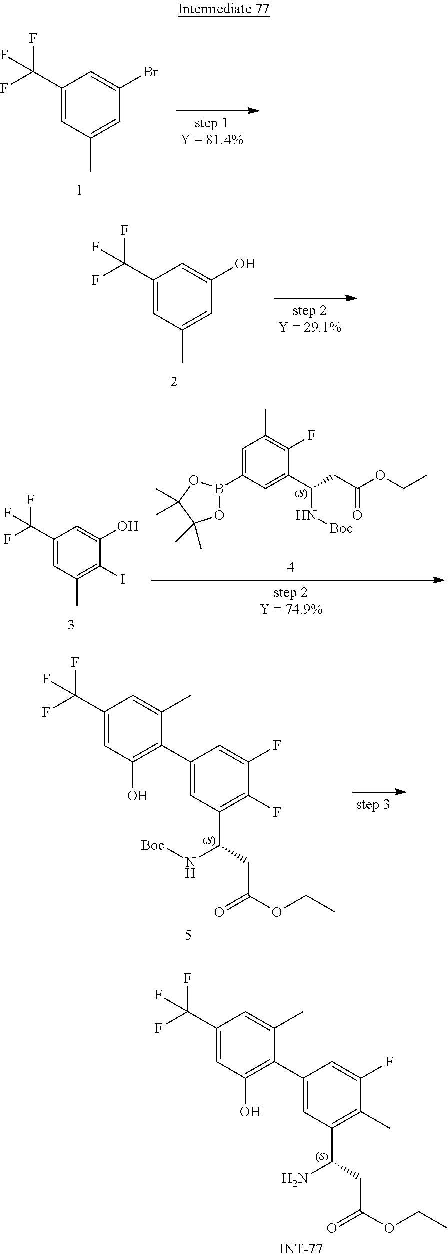

### Step 1: Preparation of 3-methyl-5-(trifluoromethyl)phenol

To a stirred solution of 1-bromo-3-methyl-5-(trifluoromethyl)benzene (5.00 g, 20.91 mmol, 1.00 equiv.) and LiOH·H₂O (2.63 g, 62.75 mmol, 3.00 equiv.) in 1,4-dioxane (50 mL) and H₂O (5 mL) was added Pd₂(dba)₃ (0.38 g, 0.41 mmol, 0.02 equiv.) and 5-(di-tert-butylphosphanyl)-1′,3′,5′-triphenyl-1′H-1,4′-bipyrazole (0.42 g, 0.83 mmol, 0.04 equiv.) at room temperature under nitrogen atmosphere. The resulting mixture was stirred overnight at 100° C. under nitrogen atmosphere and then cooled to room temperature. The resulting mixture was filtered and the filter cake was washed with EtOAc (3×100 mL). The combined filtrate was washed with HCl (aq. 1.5 M), dried over anhydrous Na₂SO₄, filtered, and concentrated. The residue was purified by silica gel column chromatography (petroleum ether/EtOAc (0 to 15% gradient in 10 min) to afford 3-methyl-5-(trifluoromethyl)phenol (3.00 g, 81.4%) as a yellow oil. ¹H NMR (300 MHz, CDCl₃) δ 9.49-8.76 (m, 1H), 7.04-6.96 (m, 1H), 6.91 (t, J=2.1 Hz, 1H), 6.84 (t, J=1.9 Hz, 1H), 2.36 (s, 3H). ¹⁹F NMR (282 MHz, CDCl₃) δ −62.75.

### Step 2: Preparation of 2-iodo-3-methyl-5-(trifluoromethyl)phenol

To a solution of 3-methyl-5-(trifluoromethyl)phenol (3.00 g, 17.03 mmol, 1.00 equiv.) in toluene (80 mL) was added sodium hydride (60% in oil, 1.36 g, 34.06 mmol, 2.00 equiv.) at 0° C. The mixture was stirred for 30 min at 0° C. I₂ (4.32 g, 17.032 mmol, 1.00 equiv.) was added and the mixture was stirred for 3 h and then quenched with water. The resulting mixture was extracted with CH₂Cl₂ (3×80 mL). The combined organic layers were washed with brine (1×30 mL), dried over anhydrous Na₂SO₄, filtered, and concentrated. The residue was purified by silica gel column chromatography (petroleum ether/CH₂Cl₂ (0 to 30% gradient in 20 min) to afford 2-iodo-3-methyl-5-(trifluoromethyl)phenol (1.50 g, 29.1%) as a white solid. ¹H NMR (400 MHz, CDCl₃) δ 7.01 (s, 2H), 5.70 (s, 1H), 2.51 (s, 3H). ¹⁹F NMR (377 MHz, CDCl₃) δ −63.04.

### Step 3: Preparation of ethyl (3S)-3-[(tert-butoxycarbonyl)amino]-3-[4-fluoro-2′-hydroxy-5,6′-dimethyl-4′-(trifluoromethyl)-[1,1′-biphenyl]-3-yl]propanoate

To a stirred solution of 2-iodo-3-methyl-5-(trifluoromethyl)phenol (600 mg, 1.99 mmol, 1.00 equiv.) and ethyl (3S)-3-[(tert-butoxycarbonyl)amino]-3-[2-fluoro-3-methyl-5-(4,4,5,5-tetramethyl-1,3,2-dioxaborolan-2-yl)phenyl]propanoate (31 mg, 0.07 mmol, 1.05 equiv.) in 1,4-dioxane (8 mL) and H₂O (0.8 mL) were added Pd(dppf)Cl₂ (145 mg, 0.20 mmol, 0.10 equiv.) and K₂CO₃ (824 mg, 5.96 mmol, 3.00 equiv.) dropwise at room temperature under nitrogen atmosphere. The resulting mixture was stirred for 2 h at 100° C. under nitrogen atmosphere, and then cooled to room temperature and concentrated. The residue was purified by silica gel column chromatography (petroleum ether/EtOAc (0% to 50% in 20 min) to afford ethyl (3S)-3-[(tert-butoxycarbonyl)amino]-3-[4-fluoro-2′-hydroxy-5,6′-dimethyl-4′-(trifluoromethyl)-[1,1′-biphenyl]-3-yl]propanoate (743 mg, 74.9%) as a white solid. LC-MS: (ES−H, m/z) [M+H]⁺=500.1. ¹H NMR (400 MHz, DMSO-d₆) δ 9.86 (s, 1H), 7.49 (d, J=8.8 Hz, 1H), 7.11-7.04 (m, 3H), 7.02-6.97 (m, 1H), 5.45-5.09 (m, 1H), 4.04 (qd, J=7.1, 5.3 Hz, 2H), 2.66 (d, J=7.3 Hz, 2H), 2.25 (d, J=1.9 Hz, 3H), 2.03 (s, 3H), 1.34 (s, 9H), 1.13 (t, J=7.1 Hz, 3H).

### Step 4: Preparation of ethyl (3S)-3-amino-3-[4-fluoro-2′-hydroxy-5,6′-dimethyl-4′-(trifluoromethyl)-[1,1′-biphenyl]-3-yl]propanoate

A solution of ethyl (3S)-3-[(tert-butoxycarbonyl)amino]-3-[4-fluoro-2′-hydroxy-5,6′-dimethyl-4′-(trifluoromethyl)-[1,1′-biphenyl]-3-yl]propanoate (648 mg, 1.30 mmol, 1.00 equiv.) and HCl (3 mL, 4 M in dioxane) in CH₂Cl₂ (3 mL) was stirred for 1 h at room temperature under nitrogen atmosphere and the concentrated. The resulting residue was diluted with THF (5 mL) and concentrated to afford ethyl (3S)-3-amino-3-[4-fluoro-2′-hydroxy-5,6′-dimethyl-4′-(trifluoromethyl)-[1,1′-biphenyl]-3-yl]propanoate (687 mg, crude) as a yellow solid. LC-MS: (ES−H, m/z) [M+H]⁺=400.1.

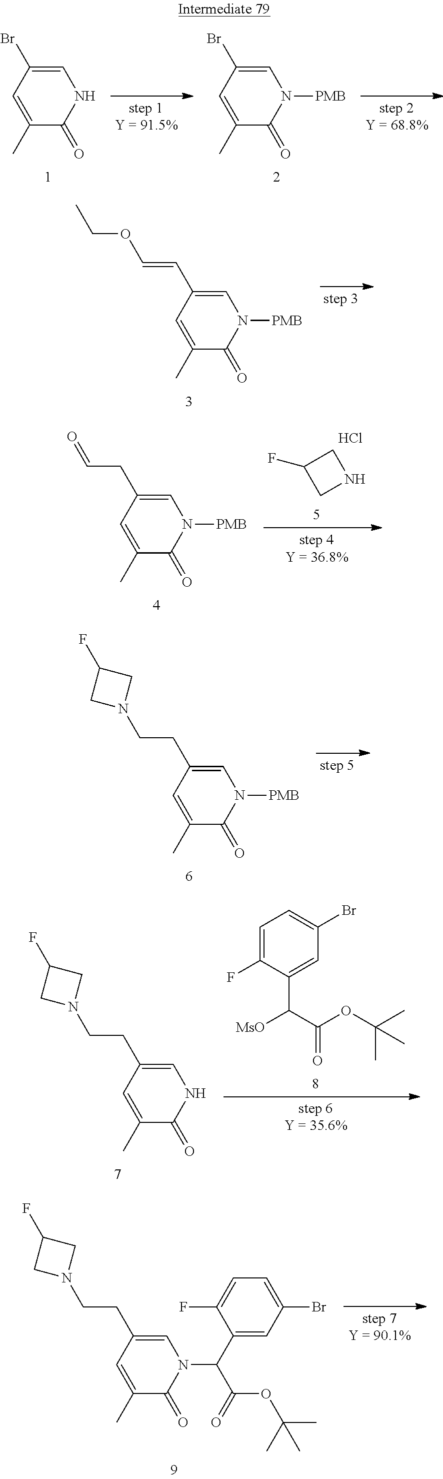

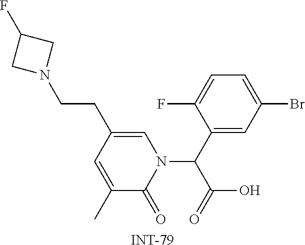

### Step 1: Preparation of 5-bromo-1-[(4-methoxyphenyl)methyl]-3-methylpyridin-2-one

To a stirred mixture of 5-bromo-3-methyl-1H-pyridin-2-one (10.00 g, 53.18 mmol, 1.00 equiv.) and PMBCl (12.49 g, 79.77 mmol, 1.50 equiv.) in DMF (100 mL) was added Cs₂CO₃ (34.66 g, 106.37 mmol, 2.00 equiv.) in portions at 0° C. The reaction mixture was stirred overnight at 60° C. under nitrogen atmosphere and then cooled to room temperature. The resulting mixture was diluted with water (100 mL) and extracted with EtOAc (3×200 mL). The combined organic layers were washed with brine (3×100 mL), dried over anhydrous Na₂SO₄, filtered, and concentrated. The residue was purified by silica gel column chromatography (petroleum ether/EtOAc (0% to 30% gradient in 25 min) to afford 5-bromo-1-[(4-methoxyphenyl)methyl]-3-methylpyridin-2-one (15.00 g, 91.5%) as a white solid. LC-MS: (ES+H, m/z) [M+H]⁺=308.15. ¹H NMR (400 MHz, DMSO-d₆) δ 7.99 (dd, J=2.8, 0.9 Hz, 1H), 7.44 (dq, J=2.4, 1.1 Hz, 1H), 7.35-7.26 (m, 2H), 6.91-6.87 (m, 2H), 5.01 (d, J=13.1 Hz, 2H), 3.72 (s, 3H), 2.00 (t, J=0.9 Hz, 3H).

### Step 2: Preparation of 5-[(E)-2-ethoxyethenyl]-1-[(4-methoxyphenyl)methyl]-3-methylpyridin-2-one

To a stirred mixture of 5-bromo-1-[(4-methoxyphenyl)methyl]-3-methylpyridin-2-one (13.00 g, 42.18 mmol, 1.00 equiv.) and 2-[(E)-2-ethoxyethenyl]-4,4,5,5-tetramethyl-1,3,2-dioxaborolane (9.19 g, 46.40 mmol, 1.10 equiv.) in 1,4-dioxane (130 mL) and H₂O (13 mL) was added Cs₂CO₃ (41.23 g, 126.55 mmol, 3.00 equiv.) and Pd(dppf)Cl₂ (3.09 g, 4.21 mmol, 0.10 equiv.) in portions at room temperature. The reaction mixture was stirred for 4 h at 80° C. under nitrogen atmosphere and then cooled to room temperature. The resulting mixture was diluted with water (100 mL) and extracted with EtOAc (3×100 mL). The combined organic layers were washed with brine (3×50 mL), dried over anhydrous Na₂SO₄, filtered, and concentrated. The residue was purified by silica gel column chromatography (petroleum ether/EtOAc (0% to 30% gradient in 25 min) to afford 5-[(E)-2-ethoxyethenyl]-1-[(4-methoxyphenyl)methyl]-3-methylpyridin-2-one (8.70 g, 68.8%) as a yellow oil. LC-MS: (ES+H, m/z) [M+H]⁺=300.1. ¹H NMR (300 MHz, DMSO-d₆) δ 7.48 (dd, J=2.6, 1.3 Hz, 1H), 7.44 (d, J=2.5 Hz, 1H), 7.31-7.23 (m, 2H), 6.89 (dq, J=9.6, 3.1, 2.5 Hz, 3H), 5.57 (d, J=13.0 Hz, 1H), 4.98 (s, 2H), 3.80 (q, J=7.0 Hz, 2H), 3.72 (s, 3H), 2.04-2.00 (m, 3H), 1.22 (t, J=7.0 Hz, 3H).

### Step 3: Preparation of 2-{1-[(4-methoxyphenyl)methyl]-5-methyl-6-oxopyridin-3-yl}acetaldehyde

To a stirred mixture of 5-[(E)-2-ethoxyethenyl]-1-[(4-methoxyphenyl)methyl]-3-methylpyridin-2-one (9.00 g, 30.06 mmol, 1.00 equiv.) in CH₂Cl₂ (90 mL) was added CF₃COOH (45 mL) dropwise at 0° C. The resulting mixture was stirred for 4 h at room temperature and then basified to pH 8 with saturated NaHCO₃ (aq.), and extracted with EtOAc (3×100 mL). The combined organic layers were washed with water (2×50 mL), dried over anhydrous Na₂SO₄, filtered, and concentrated to afford 2-{1-[(4-methoxyphenyl)methyl]-5-methyl-6-oxopyridin-3-yl}acetaldehyde (5.50 g, crude) as a yellow oil. LC-MS: (ES+H, m/z) [M+H]⁺=272.1.

### Step 4: Preparation of 5-[2-(3-fluoroazetidin-1-yl)ethyl]-1-[(4-methoxyphenyl)methyl]-3-methylpyridin-2-one

To a stirred mixture of 2-{1-[(4-methoxyphenyl)methyl]-5-methyl-6-oxopyridin-3-yl}acetaldehyde (5.50 g, 20.27 mmol, 1.00 equiv.) and 3-fluoroazetidine·HCl salt (2.47 g, 22.29 mmol, 1.10 equiv.) in EtOH (60 mL) was added AcOH (1.22 g, 20.27 mmol, 1.00 equiv.) in portions at room temperature. The resulting mixture was stirred for 1 h at room temperature. To the above mixture was added NaBH₃CN (2.55 g, 40.54 mmol, 2.00 equiv.) in portions at 0° C. The resulting mixture was stirred overnight at room temperature. The reaction was quenched by the addition of water (50 mL) at 0° C. and then extracted with EtOAc (3×100 mL). The combined organic layers were washed with brine (2×50 mL), dried over anhydrous Na₂SO₄, filtered, and concentrated. The residue was purified by silica gel column chromatography (CH₂Cl₂/MeOH (0% to 10% gradient in 25 min) to afford 5-[2-(3-fluoroazetidin-1-yl)ethyl]-1-[(4-methoxyphenyl)methyl]-3-methylpyridin-2-one (3.00 g, 36.8%) as a white solid. LC-MS: (ES+H, m/z) [M+H]⁺=331.05. ¹H NMR (400 MHz, DMSO-d₆) δ 3.72 (s, 3H), 3.69-3.58 (m, 2H), 3.19 (d, J=23.9 Hz, 2H), 2.66 (t, J=7.3 Hz, 2H), 2.31 (t, J=7.3 Hz, 2H), 1.98 (s, 3H), 7.48-7.43 (m, 1H), 7.32-7.25 (m, 2H), 7.23 (dd, J=2.6, 1.3 Hz, 1H), 6.89 (d, J=2.1 Hz, 1H), 6.87 (d, J=2.0 Hz, 1H), 5.26-5.03 (m, 1H), 4.97 (s, 2H).

### Step 5: Preparation of 5-[2-(3-fluoroazetidin-1-yl)ethyl]-3-methyl-1H-pyridin-2-one

To a stirred mixture of 5-[2-(3-fluoroazetidin-1-yl)ethyl]-1-[(4-methoxyphenyl)methyl]-3-methylpyridin-2-one (2 g, 6.05 mmol, 1.00 equiv.) in CF₃CH₂OH (30 mL) was added Pd(OH)₂ (0.85 g, 20% wt), Pd/C (0.64 g, 20% wt) in portions at 0° C. The resulting mixture was stirred overnight at room temperature under hydrogen atmosphere. The resulting mixture was filtered and the filter cake was washed with MeOH (3×20 mL). The filtrate was concentrated to afford 5-[2-(3-fluoroazetidin-1-yl)ethyl]-3-methyl-1H-pyridin-2-one (800 mg, crude) as a white solid. LC-MS: (ES+H, m/z) [M+H]⁺=211.00.

### Step 6: Preparation of tert-butyl 2-(5-bromo-2-fluorophenyl)-2-{5-[2-(3-fluoroazetidin-1-yl)ethyl]-3-methyl-2-oxopyridin-1-yl}acetate

To a stirred mixture of 5-[2-(3-fluoroazetidin-1-yl)ethyl]-3-methyl-1H-pyridin-2-one (600 mg, 2.85 mmol, 1.00 equiv.) and tert-butyl 2-(5-bromo-2-fluorophenyl)-2-(methanesulfonyloxy)acetate (1.20 g, 3.13 mmol, 1.10 equiv.) in MeCN (10 mL) was added K₂CO₃ (788 mg, 5.70 mmol, 2.00 equiv.) in portions at room temperature. The resulting mixture was stirred for 2 h at 80° C. under nitrogen atmosphere. The mixture was allowed to cool down to room temperature and diluted with water (50 mL). The resulting mixture was extracted with EtOAc (3×50 mL) and the combined organic layers were washed with brine (2×20 mL), dried over anhydrous Na₂SO₄, filtered, and concentrated. The residue was purified by reversed-phase flash chromatography (C18 gel; mobile phase, MeCN in water (10 mmol/L NH₄HCO₃), 0% to 100% gradient in 30 min; detector, UV 220 nm) to provide tert-butyl 2-(5-bromo-2-fluorophenyl)-2-{5-[2-(3-fluoroazetidin-1-yl)ethyl]-3-methyl-2-oxopyridin-1-yl}acetate (800 mg, 56.3%) as a yellow solid. LC-MS: (ES+H, m/z) [M+H]⁺=497.10. ¹H NMR (400 MHz, DMSO-d₆) δ 7.67 (ddd, J=8.8, 4.6, 2.6 Hz, 1H), 7.53 (dd, J=6.5, 2.5 Hz, 1H), 7.37-7.27 (m, 2H), 7.21-7.14 (m, 1H), 6.46 (s, 1H), 5.08 (dp, J=57.7, 5.0 Hz, 1H), 3.57-3.45 (m, 2H), 3.06 (ddd, J=23.8, 8.8, 4.5 Hz, 2H), 2.58 (t, J=7.0 Hz, 2H), 2.31 (t, J=7.0 Hz, 2H), 2.01 (s, 3H), 1.40 (s, 9H).

### Step 7: Preparation of (5-bromo-2-fluorophenyl)({5-[2-(3-fluoroazetidin-1-yl)ethyl]-3-methyl-2-oxopyridin-1-yl})acetic acid

To a stirred mixture of tert-butyl 2-(5-bromo-2-fluorophenyl)-2-{5-[2-(3-fluoroazetidin-1-yl)ethyl]-3-methyl-2-oxopyridin-1-yl}acetate (750 mg, 1.508 mmol, 1.00 equiv.) in CH₂Cl₂ (10 mL) was added CF₃COOH (5 mL) dropwise at 0° C. The resulting mixture was stirred for 4 h at room temperature and then was concentrated. The residue was purified by reversed-phase flash chromatography (C18 gel; mobile phase, MeCN in water (0.1% NH₃·H₂O), 0% to 100% gradient in 30 min; detector, UV 220 nm) to afford (5-bromo-2-fluorophenyl)({5-[2-(3-fluoroazetidin-1-yl)ethyl]-3-methyl-2-oxopyridin-1-yl})acetic acid (600 mg, 90.1%) as a yellow solid. LC-MS: (ES+H, m/z) [M+H]⁺=441.00. ¹H NMR (400 MHz, DMSO-d₆) δ 10.31 (d, J=244.7 Hz, 1H), 7.73-7.55 (m, 2H), 7.33-7.24 (m, 2H), 6.56 (s, 1H), 5.37 (d, J=57.1 Hz, 1H), 4.48-4.19 (m, 3H), 3.44 (d, J=7.0 Hz, 2H), 2.55 (d, J=3.2 Hz, 2H), 2.07 (s, 3H), 2.03 (s, 2H).

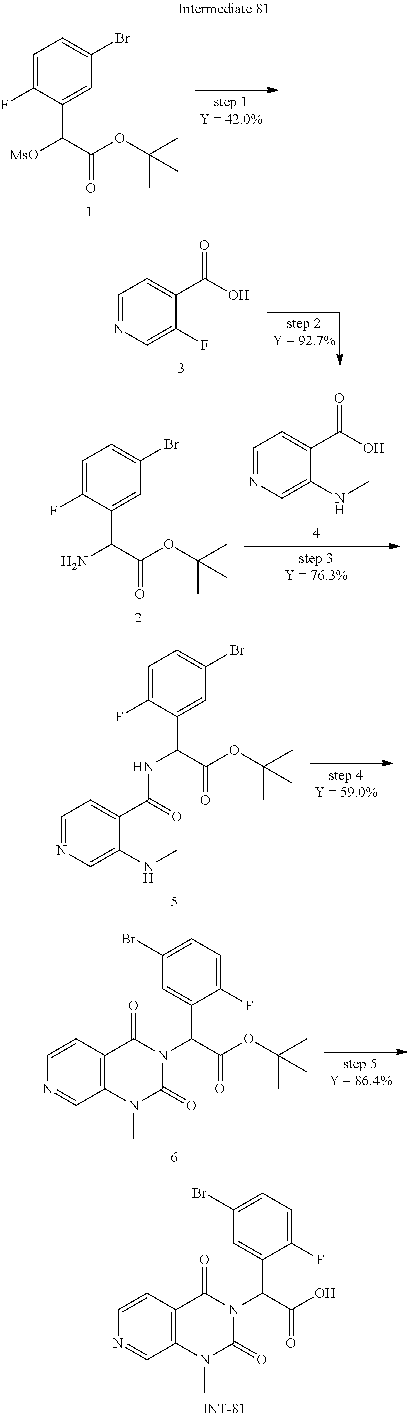

### Step 1: Preparation of tert-butyl 2-amino-2-(5-bromo-2-fluorophenyl)acetate

A solution of tert-butyl 2-(5-bromo-2-fluorophenyl)-2-(methanesulfonyloxy)acetate (3.00 g, 7.82 mmol, 1.00 equiv.) and NH₃·H₂O (24 mL) in MeOH (72 mL) was stirred for 3 h at 60° C. under nitrogen atmosphere and then cooled to room temperature. The resulting mixture was diluted with water (50 mL) and extracted with CH₂Cl₂ (4×80 mL). The combined organic layers were washed with brine (1×15 mL), dried over anhydrous Na₂SO₄, filtered, and concentrated. The residue was purified by silica gel column chromatography (petroleum ether/EtOAc (10% to 40% gradient in 15 min) to afford tert-butyl 2-amino-2-(5-bromo-2-fluorophenyl)acetate (1.00 g, 42.0%) as light yellow oil. LC-MS: (ES+H, m/z) [M+H−tBu]⁺=247.80. ¹H NMR (400 MHz, DMSO-d₆) δ 7.66 (dd, J=6.5, 2.6 Hz, 1H), 7.53-7.47 (m, 1H), 7.17 (dd, J=9.9, 8.7 Hz, 1H), 4.59 (s, 1H), 2.34 (s, 2H), 1.34 (s, 9H). ¹⁹F NMR (377 MHz, DMSO-d₆) δ −119.9.

### Step 2: Preparation of 3-(methylamino)pyridine-4-carboxylic acid

A solution of 3-fluoropyridine-4-carboxylic acid (11.00 g, 77.95 mmol, 1.00 equiv.) and methylamine (77.48 g, 623.66 mmol, 8.00 equiv., 25%) in 1,4-dioxane (24 mL) was stirred for 14 h at 140° C. under nitrogen atmosphere and then cooled to room temperature. The resulting mixture was filtered and the filter cake was washed with H₂O (2×10 mL). The filtrate was concentrated under reduced pressure to provide 3-(methylamino)pyridine-4-carboxylic acid (11.00 g, 92.7%) as a yellow solid. LC-MS: (ES+H, m/z) [M+H]⁺=153.10. ¹H NMR (400 MHz, DMSO-d₆) δ 8.21 (s, 1H), 7.84 (d, J=5.0 Hz, 1H), 7.58-7.51 (m, 1H), 2.92 (s, 3H).

### Step 3: Preparation of tert-butyl 2-(5-bromo-2-fluorophenyl)-2-{[3-(methylamino)pyridin-4-yl]formamido}acetate

To a stirred solution of tert-butyl 2-amino-2-(5-bromo-2-fluorophenyl)acetate (1.00 g, 3.28 mmol, 1.00 equiv.) and 3-(methylamino)pyridine-4-carboxylic acid (0.55 g, 3.61 mmol, 1.10 equiv.) in MeCN (10 mL) were added TCFH (2.77 g, 9.86 mmol, 3.00 equiv.) and NMI (1.08 g, 13.15 mmol, 4.00 equiv.) at room temperature under nitrogen atmosphere. The resulting mixture was stirred for 2 h at room temperature, and then diluted with water (50 mL) and extracted with EtOAc (3×60 mL). The combined organic layers were washed with brine (1×10 mL), dried over anhydrous Na₂SO₄, filtered, and concentrated. The residue was purified by silica gel column chromatography (petroleum ether/EtOAc (10% to 50% gradient in 20 min) to afford tert-butyl 2-(5-bromo-2-fluorophenyl)-2-{[3-(methylamino)pyridin-4-yl]formamido}acetate (1.10 g, 76.3%) as yellow oil. LC-MS: (ES+H, m/z) [M+H]⁺=438.05. ¹H NMR (300 MHz, DMSO-d₆) δ 9.29 (d, J=7.7 Hz, 1H), 8.14 (s, 1H), 7.88 (d, J=5.0 Hz, 1H), 7.71 (dd, J=6.4, 2.6 Hz, 1H), 7.61 (ddd, J=8.8, 4.6, 2.5 Hz, 1H), 7.52 (d, J=5.0 Hz, 1H), 7.28 (dd, J=9.8, 8.8 Hz, 1H), 7.17 (d, J=5.3 Hz, 1H), 5.81 (d, J=7.7 Hz, 1H), 2.85 (d, J=4.9 Hz, 3H), 1.38 (s, 9H).

### Step 4: Preparation of tert-butyl 2-(5-bromo-2-fluorophenyl)-2-{1-methyl-2,4-dioxopyrido[3,4-d]pyrimidin-3-yl}acetate

To a solution of tert-butyl 2-(5-bromo-2-fluorophenyl)-2-{[3-(methylamino)pyridin-4-yl]formamido}acetate (800 mg, 1.82 mmol, 1.00 equiv.) in DMF (10 mL) was added sodium hydride (60% in oil, 146 mg, 2.00 equiv.) at 0° C. The mixture was stirred for 1 h and then 1,1′-carbonyldiimidazole (444 mg, 2.74 mmol, 1.50 equiv.) was added. After 1 h at room temperature, the reaction was quenched with 0° C. and extracted with EtOAc (3×80 mL). The combined organic layers were washed with brine (1×10 mL), dried over anhydrous Na₂SO₄, filtered, and concentrated. The residue was purified by reversed-phase flash chromatography (C18 gel; mobile phase, MeCN in water (0.1% NH₃·H₂O), 10% to 50% gradient in 10 min; detector, UV 220 nm) to provide tert-butyl 2-(5-bromo-2-fluorophenyl)-2-{1-methyl-2,4-dioxopyrido[3,4-d]pyrimidin-3-yl}acetate (500 mg, 59.0%) as light yellow oil. LC-MS: (ES+H, m/z) [M+H]⁺=464.0. ¹H NMR (400 MHz, DMSO-d₆) δ 9.00 (s, 1H), 8.59 (d, J=5.0 Hz, 1H), 7.95 (dd, J=5.0, 0.7 Hz, 1H), 7.67 (dd, J=6.6, 2.5 Hz, 1H), 7.61-7.55 (m, 1H), 7.24 (dd, J=10.1, 8.8 Hz, 1H), 6.59 (s, 1H), 3.63 (s, 3H), 1.36 (s, 9H). ¹⁹F NMR (377 MHz, DMSO-d₆) δ −117.08.

### Step 5: Preparation of (5-bromo-2-fluorophenyl)({1-methyl-2,4-dioxopyrido[3,4-d]pyrimidin-3-yl})acetic acid

To a stirred solution of tert-butyl 2-(5-bromo-2-fluorophenyl)-2-{1-methyl-2,4-dioxopyrido[3,4-d]pyrimidin-3-yl}acetate (500 mg, 1.07 mmol, 1.00 equiv.) in THF (4 mL) were added CF₃COOH (8 mL) dropwise at 0° C. under nitrogen atmosphere. The resulting mixture was stirred overnight at 40° C. and then cooled to room temperature and concentrated. The residue was purified by reversed-phase flash chromatography (C18 gel; mobile phase, MeCN in water, 0% to 50% gradient in 20 min; detector, UV 220 nm) to provide (5-bromo-2-fluorophenyl)({1-methyl-2,4-dioxopyrido[3,4-d]pyrimidin-3-yl})acetic acid (380 mg, 86.4%) as a light yellow oil. LC-MS: (ES+H, m/z) [M+H]⁺=407.8.

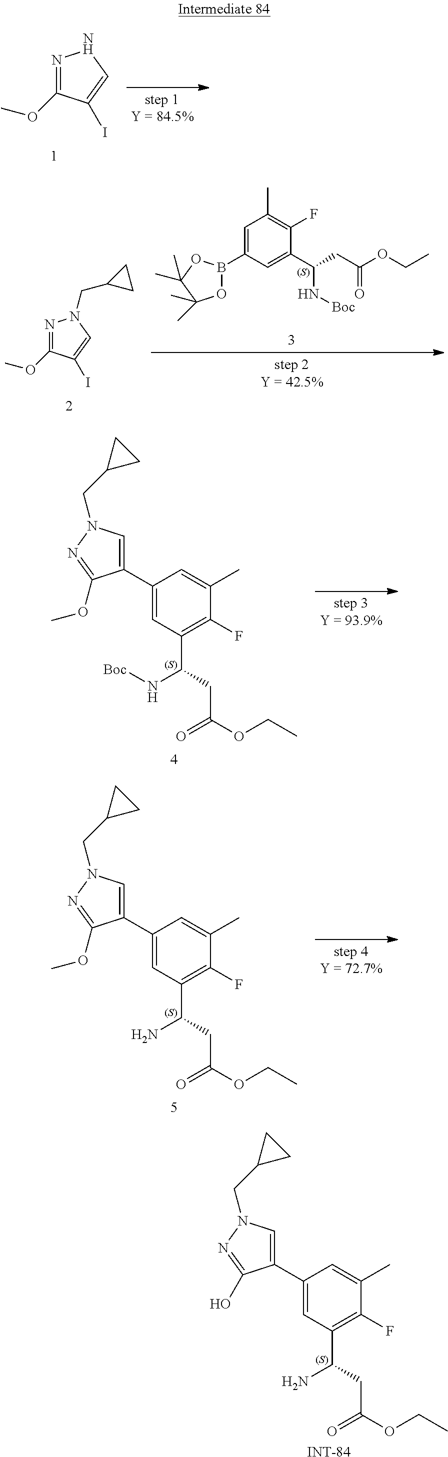

### Step 1: Preparation of 1-(cyclopropylmethyl)-4-iodo-3-methoxypyrazole

To a stirred solution of 4-iodo-3-methoxy-1H-pyrazole (2.00 g, 8.92 mmol, 1.00 equiv.) in MeCN (15 mL) were added cyclopropyl bromide (2.16 g, 17.85 mmol, 2.00 equiv.) and Cs₂CO₃ (5.82 g, 17.85 mmol, 2.00 equiv.) at room temperature. The resulting mixture was stirred for 3 h at 60° C. under nitrogen atmosphere, and then cooled to room temperature and concentrated. The resulting mixture was diluted with water (20 mL), and extracted with EtOAc (3×50 mL). The combined organic layers were washed with brine (2×20 mL), dried over anhydrous Na₂SO₄, filtered, and concentrated. The residue was purified by silica gel column chromatography (petroleum ether/CH₂Cl₂ (10% to 30% in 20 min) to afford 1-(cyclopropylmethyl)-4-iodo-3-methoxypyrazole (2.10 g, 84.5%) as colorless oil. LC-MS: (ES+H, m/z) [M+H]⁺=278.9. ¹H NMR (300 MHz, DMSO-d₆) δ 7.73 (s, 1H), 3.83-3.77 (m, 5H), 1.27-1.11 (m, 1H), 0.55-0.46 (m, 2H), 0.36-0.27 (m, 2H).

### Step 2: Preparation of ethyl (3S)-3-[(tert-butoxycarbonyl)amino]-3-{5-[1-(cyclopropylmethyl)-3-methoxypyrazol-4-yl]-2-fluoro-3-methylphenyl}propanoate

To a stirred solution of 1-(cyclopropylmethyl)-4-iodo-3-methoxypyrazole (900 mg, 3.23 mmol, 1.00 equiv.) and ethyl (3S)-3-[(tert-butoxycarbonyl)amino]-3-[2-fluoro-3-methyl-5-(4,4,5,5-tetramethyl-1,3,2-dioxaborolan-2-yl)phenyl]propanoate (649 mg, 1.43 mmol, 1.00 equiv.) in 1,4-dioxane/H₂O=3:1 (15 mL) were added K₂CO₃ (1.34 g, 9.70 mmol, 3.00 equiv.) and Pd(dppf)Cl₂ (264 mg, 0.32 mmol, 0.10 equiv.) at room temperature under air atmosphere. The resulting mixture was stirred for 2 h at 100° C. under nitrogen atmosphere and then cooled to room temperature. The resulting mixture was diluted with water (50 mL) an extracted with EtOAc (3×50 mL). The combined organic layers were washed with brine (2×50 mL), dried over anhydrous Na₂SO₄, filtered, and concentrated. The residue was purified by silica gel column chromatography (petroleum ether/EtOAc (10%-30% in 20 min) to afford ethyl (3S)-3-[(tert-butoxycarbonyl)amino]-3-{5-[1-(cyclopropylmethyl)-3-methoxypyrazol-4-yl]-2-fluoro-3-methylphenyl}propanoate (655 mg, 42.5%) as colorless oil. LC-MS: (ES+H, m/z) [M+H]⁺=476.25. ¹H NMR (400 MHz, CDCl₃) δ 7.52 (s, 1H), 7.39-7.29 (m, 2H), 5.62-5.40 (m, 1H), 4.17-4.02 (m, 3H), 4.00 (s, 3H), 3.84 (d, J=7.0 Hz, 2H), 2.27 (d, J=2.1 Hz, 3H), 2.05 (s, 2H), 1.43 (s, 9H), 1.18 (t, J=7.1 Hz, 4H), 0.72-0.59 (m, 2H), 0.37 (t, J=5.3 Hz, 2H). ¹⁹F NMR (377 MHz, CDCl₃) δ −126.72.

### Step 3: Preparation of ethyl (3S)-3-amino-3-{5-[1-(cyclopropylmethyl)-3-methoxypyrazol-4-yl]-2-fluoro-3-methylphenyl}propanoate

To a stirred solution of ethyl (3S)-3-[(tert-butoxycarbonyl)amino]-3-{5-[1-(cyclopropylmethyl)-3-methoxypyrazol-4-yl]-2-fluoro-3-methylphenyl}propanoate (580 mg, 1.22 mmol, 1.00 equiv.) in CH₂Cl₂ (5 mL) was added CF₃COOH (5 mL) at room temperature under nitrogen atmosphere. The resulting mixture was stirred for 2 h at room temperature and then concentrated. The residue was purified by reversed-phase flash chromatography (C18 XBridge; mobile phase, MeCN in water (0.1% NH₃·H₂O), 10% to 50% gradient in 10 min; detector, UV 254 nm) to provide ethyl (3S)-3-amino-3-{5-[1-(cyclopropylmethyl)-3-methoxypyrazol-4-yl]-2-fluoro-3-methylphenyl}propanoate (430 mg, 93.9%) as colorless oil. LC-MS: (ES+H, m/z) [M+H]⁺=376.15. ¹H NMR (400 MHz, DMSO-d₆) δ 7.99 (s, 1H), 7.53 (dd, J=6.8, 2.3 Hz, 1H), 7.38 (dd, J=7.3, 2.3 Hz, 1H), 4.44 (t, J=7.0 Hz, 1H), 4.09-3.98 (m, 2H), 3.89 (s, 3H), 3.81 (d, J=7.0 Hz, 2H), 2.64-2.54 (m, 2H), 2.21 (d, J=2.0 Hz, 3H), 1.30-1.17 (m, 1H), 1.13 (t, J=7.1 Hz, 3H), 0.60-0.48 (m, 2H), 0.42-0.31 (m, 2H). ¹⁹F NMR (377 MHz, DMSO-d₆) δ −128.66.

### Step 4: Preparation of ethyl (3S)-3-amino-3-{5-[1-(cyclopropylmethyl)-3-hydroxypyrazol-4-yl]-2-fluoro-3-methylphenyl}propanoate

To a stirred solution of ethyl (3S)-3-amino-3-{5-[1-(cyclopropylmethyl)-3-methoxypyrazol-4-yl]-2-fluoro-3-methylphenyl}propanoate (400 mg, 1.06 mmol, 1.00 equiv.) in MeCN (10 mL) was added TMSI (4.26 mL, 4.26 mmol, 4.00 equiv.) dropwise at room temperature under nitrogen atmosphere. The resulting mixture was stirred for 4 h at 80° C. under nitrogen atmosphere and then cooled to room temperature and diluted with water (30 mL). The resulting mixture was extracted with EtOAc (3×30 mL) and the combined organic layers were washed with brine (2×20 mL), dried over anhydrous Na₂SO₄, filtered, and concentrated. The residue was purified by reversed-phase flash chromatography (C18 XBridge; mobile phase, MeCN in water, 10% to 50% gradient in 10 min; detector, UV 254 nm) to provide ethyl (3S)-3-amino-3-{5-[1-(cyclopropylmethyl)-3-hydroxypyrazol-4-yl]-2-fluoro-3-methylphenyl}propanoate (280 mg, 72.7%) as a yellow solid. LC-MS: (ES+H, m/z) [M+H]⁺=362.20. ¹H NMR (400 MHz, DMSO-d₆) δ 10.38 (s, 1H), 7.88 (s, 1H), 7.61 (d, J=6.4 Hz, 1H), 7.56 (d, J=7.0 Hz, 1H), 4.74 (s, 1H), 4.10-3.97 (m, 2H), 3.76 (d, J=7.0 Hz, 2H), 2.97 (d, J=8.4 Hz, 2H), 2.24 (d, J=1.9 Hz, 3H), 1.12 (t, J=7.1 Hz, 3H), 0.84 (dd, J=8.8, 6.8 Hz, 1H), 0.58-0.50 (m, 2H), 0.38-0.30 (m, 2H). ¹⁹F NMR (377 MHz, DMSO-d₆) δ −127.11.

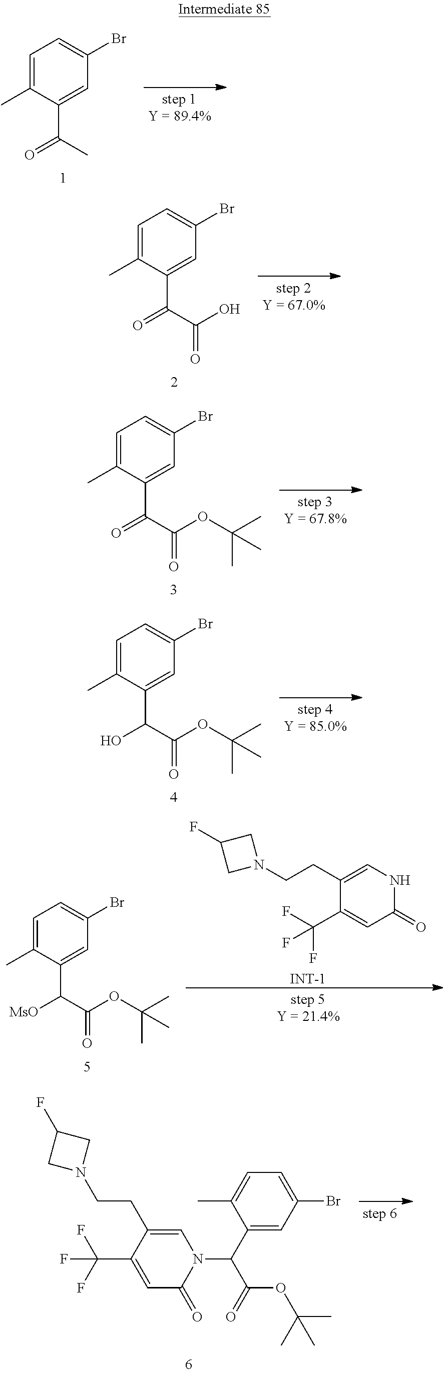

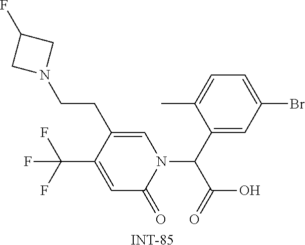

### Step 1: Preparation of 2-(5-bromo-2-methylphenyl)-2-oxoacetic acid

A mixture of 1-(5-bromo-2-methylphenyl)ethanone (5.00 g, 23.46 mmol, 1.00 equiv.) and SeO₂ (5.21 g, 46.93 mmol, 2.00 equiv.) in pyridine (50 mL) was stirred for 2 h at 100° C. under nitrogen atmosphere and then cooled to room temperature. The resulting mixture was diluted with water (100 mL), acidified to pH 2 with 3 M HCl (aq.) (150 mL) and extracted with EtOAc (500 mL). The organic layer was washed with water (3×200 mL), brine (1×200 mL), dried over anhydrous Na₂SO₄, filtered, and concentrated to provide 2-(5-bromo-2-methylphenyl)-2-oxoacetic acid (5.10 g, 89.4%) as an off-white solid. LC-MS: (ES−H, m/z) [M−H]⁻=240.8.

### Step 2: Preparation of tert-butyl 2-(5-bromo-2-methylphenyl)-2-oxoacetate

A mixture of (5-bromo-2-methylphenyl)(oxo)acetic acid (5.00 g, 20.57 mmol, 1.00 equiv.) and N,N′-diisopropyltert-butoxymethanimidamide (8.24 g, 41.14 mmol, 2.00 equiv.) in CH₂Cl₂ (20 mL) was stirred overnight at room temperature under nitrogen atmosphere and then concentrated. The residue was purified by silica gel column chromatography (PE/EtOAc (0% to 20% gradient in 25 min) to tert-butyl 2-(5-bromo-2-methylphenyl)-2-oxoacetate (4.1 g, 67.0%) as an off-white solid. LC-MS: (ES−H, m/z) [M−H−tBu]⁻=241.0.

### Step 3: Preparation of tert-butyl 2-(5-bromo-2-methylphenyl)-2-hydroxyacetate

A mixture of tert-butyl 2-(5-bromo-2-methylphenyl)-2-oxoacetate (4.10 g, 13.70 mmol, 1.00 equiv.) and NaBH₃CN (947 mg, 15.07 mmol, 1.10 equiv.) in CH₃CH₂OH (40 mL) CH₃COOH (4 mL) was stirred for 2 h at room temperature under nitrogen atmosphere. The reaction was quenched with water (50 mL) at 0° C. and the aqueous layer was extracted with CH₂Cl₂ (3×70 mL). The combined organic layers were concentrated and the residue was purified by silica gel column chromatography (PE/EtOAc (0% to 20% gradient in 25 min) to afford tert-butyl 2-(5-bromo-2-methylphenyl)-2-hydroxyacetate (2.80 g, 67.8%) as a white solid. ¹H NMR (300 MHz, DMSO-d₆) δ 7.51 (d, J=2.2 Hz, 1H), 7.38 (dd, J=8.1, 2.2 Hz, 1H), 7.15 (dd, J=8.1, 0.8 Hz, 1H), 6.03 (d, J=5.3 Hz, 1H), 5.14 (d, J=5.3 Hz, 1H), 2.30 (s, 3H), 1.34 (s, 9H).

### Step 4: Preparation of tert-butyl 2-(5-bromo-2-methylphenyl)-2-((methylsulfonyl)oxy)acetate

A solution of tert-butyl 2-(5-bromo-2-methylphenyl)-2-hydroxyacetate (2.80 g, 9.29 mmol, 1.00 equiv.) in CH₂Cl₂ (15 mL) was treated with Et₃N (3.88 mL, 27.89 mmol, 3.0 equiv.) for 20 min at room temperature under nitrogen atmosphere followed by the addition of MsCl (0.86 mL, 11.15 mmol, 1.20 equiv.) dropwise at 0° C. The resulting mixture was stirred for 2 h at room temperature and then quenched by the addition of water (5 mL) at 0° C. The resulting mixture was diluted with water (100 mL) and extracted with EtOAc (3×100 mL). The combined organic layers were washed with brine (1×60 mL), dried over anhydrous Na₂SO₄, filtered, and concentrated to provide tert-butyl 2-(5-bromo-2-methylphenyl)-2-((methylsulfonyl)oxy)acetate (3.00 g, 85.0%) as a white solid. The crude product was used in the next step directly without further purification.

### Step 5: Preparation of tert-butyl 2-(5-bromo-2-methylphenyl)-2-(5-(2-(3-fluoroazetidin-1-yl)ethyl)-2-oxo-4-(trifluoromethyl)pyridin-1(2H)-yl)acetate

A mixture of tert-butyl 2-(5-bromo-2-methylphenyl)-2-(methanesulfonyloxy)acetate (3.10 g, 8.17 mmol, 1.20 equiv.), 5-[2-(3-fluoroazetidin-1-yl)ethyl]-4-(trifluoromethyl)-1H-pyridin-2-one (1.80 g, 6.81 mmol, 1.00 equiv.) and K₂CO₃ (2.82 g, 20.43 mmol, 3.0 equiv.) in MeCN (20 mL) was stirred for 2 h at 80° C. under nitrogen atmosphere. The resulting mixture was concentrated under reduced pressure. The residue was purified by reversed-phase flash chromatography (C18 gel; mobile phase, MeCN in water (0.1% NH₃·H₂O), 40% to 80% gradient in 10 min; detector, UV 254 nm) to provide tert-butyl 2-(5-bromo-2-methylphenyl)-2-(5-(2-(3-fluoroazetidin-1-yl)ethyl)-2-oxo-4-(trifluoromethyl)pyridin-1(2H)-yl)acetate (800 mg, 21.4%) as an off-white solid. LC-MS: (ES+H, m/z) [M+H]⁺=547.1. ¹H NMR (300 MHz, DMSO-d₆) δ 7.65 (dd, J=8.2, 2.1 Hz, 1H), 7.40-7.33 (m, 2H), 7.26 (s, 1H), 6.88 (s, 1H), 6.39 (s, 1H), 5.10-4.79 (m, 1H), 3.37 (s, 1H), 3.31-3.22 (m, 1H), 2.90 (dd, J=24.3, 8.9, 4.9 Hz, 2H), 2.40 (d, J=13.1 Hz, 4H), 2.08 (d, J=2.8 Hz, 3H), 1.45 (s, 9H). ¹⁹F NMR (282 MHz, DMSO) δ −62.63, −176.89.

### Step 6: Preparation of 2-(5-bromo-2-methylphenyl)-2-(5-(2-(3-fluoroazetidin-1-yl)ethyl)-2-oxo-4-(trifluoromethyl)pyridin-1(2H)-yl)acetic acid

A mixture of tert-butyl 2-(5-bromo-2-methylphenyl)-2-{5-[2-(3-fluoroazetidin-1-yl)ethyl]-2-oxo-4-(trifluoromethyl)pyridin-1-yl}acetate (400 mg, 0.73 mmol, 1.00 equiv.) and TFA (3 mL, 40.38 mmol, 55.27 equiv.) in CH₂Cl₂ (1 mL) was stirred for 1 h at room temperature under nitrogen atmosphere and then concentrated under reduced pressure to provide 2-(5-bromo-2-methylphenyl)-2-(5-(2-(3-fluoroazetidin-1-yl)ethyl)-2-oxo-4-(trifluoromethyl)pyridin-1(2H)-yl)acetic acid (550 mg, crude) as an off-white solid. The crude product was used in the next step directly without further purification. LC-MS. (ES+H, m/z) [M+H]⁺=491.0.

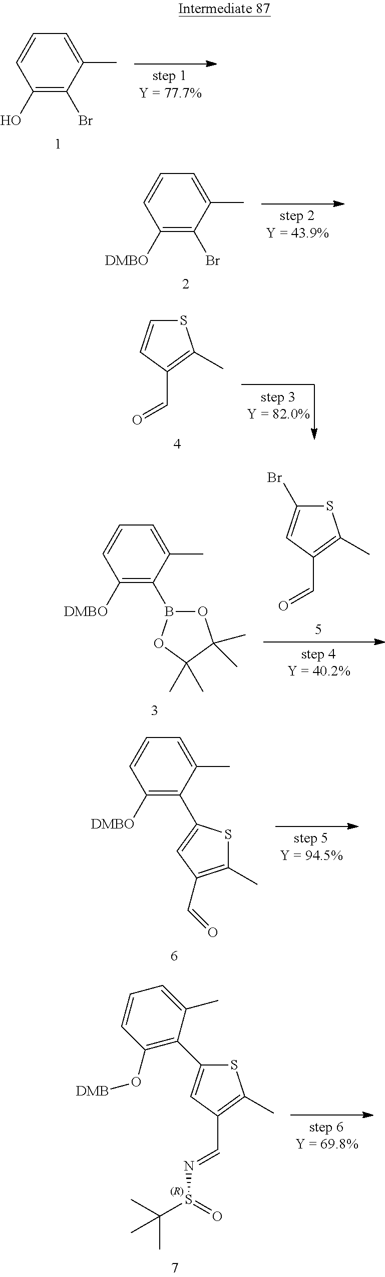

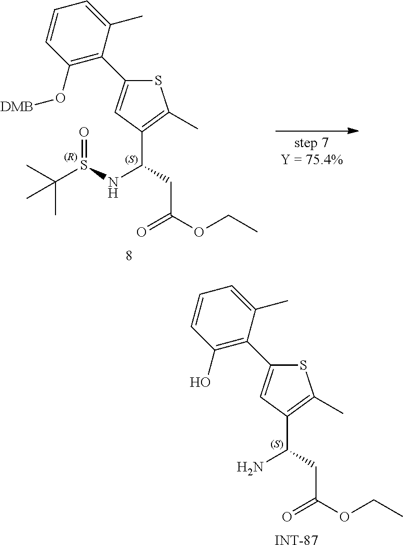

### Step 1: Preparation of 2-bromo-1-[(3,4-dimethoxyphenyl)methoxy]-3-methylbenzene

A mixture of 2-bromo-3-methylphenol (5.00 g, 26.73 mmol, 1.00 equiv.), 4-(bromomethyl)-1,2-dimethoxybenzene (6.80 g, 29.41 mmol, 1.10 equiv.) and KOH (4.50 g, 80.20 mmol, 3.00 equiv.) in MeCN (20 mL) was stirred for 2 h at 50° C. under nitrogen atmosphere and then cooled to room temperature. The resulting mixture was filtered and the filter cake was washed with EtOAc (3×50 mL). The filtrate was concentrated under reduced pressure and the residue was purified by silica gel column chromatography (petroleum ether/EtOAc (0 to 30% in 15 min) to afford 2-bromo-1-[(3,4-dimethoxyphenyl)methoxy]-3-methylbenzene (7.00 g, 77.7%) as a white solid. ¹H NMR (400 MHz, DMSO-d₆) δ 7.21 (t, J=7.9 Hz, 1H), 7.10 (d, J=1.9 Hz, 1H), 7.04-6.98 (m, 2H), 6.97-6.91 (m, 2H), 5.10 (s, 2H), 3.76 (s, 3H), 3.75 (s, 3H), 2.35 (s, 3H).

### Step 2: Preparation of 2-{2-[(3,4-dimethoxyphenyl)methoxy]-6-methylphenyl}-4,4,5,5-tetramethyl-1,3,2-dioxaborolane

A mixture of 2-bromo-1-[(3,4-dimethoxyphenyl)methoxy]-3-methylbenzene (4.00 g, 11.86 mmol, 1.00 equiv.), KOAc (3.49 g, 35.59 mmol, 3.00 equiv.), Pd(dppf)Cl₂ (0.87 g, 1.19 mmol, 0.10 equiv.) and 4,4,5,5-tetramethyl-2-(4,4,5,5-tetramethyl-1,3,2-dioxaborolan-2-yl)-1,3,2-dioxaborolane (4.52 g, 17.79 mmol, 1.50 equiv.) in 1,4-dioxane (50 mL) was stirred for 24 h at 80° C. under nitrogen atmosphere. The reaction was then cooled to room temperature, diluted with water (100 mL) and extracted with EtOAc (3×100 mL). The combined organic layers were washed with brine (3×100 mL), dried over anhydrous Na₂SO₄, filtered, and concentrated. The residue was purified by silica gel column chromatography (petroleum ether/EtOAc (0 to 30% in 20 min) to afford 2-{2-[(3,4-dimethoxyphenyl)methoxy]-6-methylphenyl}-4,4,5,5-tetramethyl-1,3,2-dioxaborolane (2 g, 43.9%) as colorless oil. ¹H NMR (400 MHz, DMSO-d₆) δ 7.18 (t, J=7.9 Hz, 1H), 7.04-6.96 (m, 2H), 6.91 (d, J=8.2 Hz, 1H), 6.81 (d, J=8.3 Hz, 1H), 6.72 (d, J=7.5 Hz, 1H), 4.95 (s, 2H), 3.74 (d, J=4.8 Hz, 6H), 2.23 (s, 3H), 1.23 (s, 12H).

### Step 3: Preparation of 5-bromo-2-methylthiophene-3-carbaldehyde

A solution of 2-methylthiophene-3-carbaldehyde (3.00 g, 23.78 mmol, 1.00 equiv.) and NBS (6.35 g, 35.67 mmol, 1.50 equiv.) in DMF (60 mL) was stirred overnight at room temperature under nitrogen atmosphere. The resulting mixture was diluted with water (75 mL) and extracted with Et₂O (3×75 mL). The combined organic layers were washed with brine (3×50 mL), dried over anhydrous Na₂SO₄, filtered, and concentrated. The residue was purified by silica gel column chromatography (petroleum ether/EtOAc (0% to 30% in 20 min) to afford 5-bromo-2-methylthiophene-3-carbaldehyde (4.00 g, 82.0%) as a white solid. ¹H NMR (400 MHz, CDCl₃) δ 9.90 (s, 1H), 7.32 (s, 1H), 2.72 (s, 3H).

### Step 4: Preparation of 5-{2-[(3,4-dimethoxyphenyl)methoxy]-6-methylphenyl}-2-methylthiophene-3-carbaldehyde

A mixture of 2-{2-[(3,4-dimethoxyphenyl)methoxy]-6-methylphenyl}-4,4,5,5-tetramethyl-1,3,2-dioxaborolane (2.50 g, 6.51 mmol, 1.00 equiv.), 5-bromo-2-methylthiophene-3-carbaldehyde (1.47 g, 7.16 mmol, 1.10 equiv.), Pd(dppf)Cl₂ (476 mg, 0.65 mmol, 0.10 equiv.) and K₂CO₃ (1.80 g, 13.01 mmol, 2.00 equiv.) in 1,4-dioxane (50 mL) and H₂O (2.5 mL) was stirred overnight at 100° C. under nitrogen atmosphere and then cooled to room temperature. The resulting mixture was diluted with water (100 mL) and extracted with EtOAc (3×100 mL). The combined organic layers were washed with brine (3×50 mL), dried over anhydrous Na₂SO₄, filtered, and concentrated. The residue was purified by silica gel column chromatography (petroleum ether/EtOAc (0 to 40% in 20 min) to afford 5-{2-[(3,4-dimethoxyphenyl)methoxy]-6-methylphenyl}-2-methylthiophene-3-carbaldehyde (1.00 g, 40.2%) as light yellow oil. LC-MS: (ES+H, m/z) [M+H]⁺=382.95. ¹H NMR (400 MHz, DMSO-d₆) δ 9.99 (s, 1H), 7.26 (t, J=8.0 Hz, 1H), 7.17 (s, 1H), 7.01 (d, J=8.3 Hz, 1H), 6.96-6.82 (m, 4H), 5.01 (s, 2H), 3.70 (d, J=15.3 Hz, 6H), 2.78 (s, 3H), 2.17 (s, 3H).

### Step 5: Preparation of (R)—N-[(5-{2-[(3,4-dimethoxyphenyl)methoxy]-6-methylphenyl}-2-methylthiophen-3-yl)methylidene]-2-methylpropane-2-sulfinamide

To a stirred solution of 5-{2-[(3,4-dimethoxyphenyl)methoxy]-6-methylphenyl}-2-methylthiophene-3-carbaldehyde (1.00 g, 2.62 mmol, 1.00 equiv.) and (R)-2-methylpropane-2-sulfinamide (349 mg, 2.88 mmol, 1.10 equiv.) in THF (20 mL) was added Ti(Oi-Pr)₄ (1.49 g, 5.23 mmol, 2.00 equiv.) dropwise at room temperature under nitrogen atmosphere. The resulting mixture was stirred for 2 days at room temperature under nitrogen atmosphere. The resulting mixture was diluted with water (30 mL). The resulting mixture was extracted with EtOAc (3×60 mL), filtered, and concentrated. The residue was purified by silica gel column chromatography (petroleum ether/EtOAc (0 to 30% in 15 min) to afford (R)—N-[(5-{2-[(3,4-dimethoxyphenyl)methoxy]-6-methylphenyl}-2-methylthiophen-3-yl)methylidene]-2-methylpropane-2-sulfinamide (1.20 g, 94.5%) as a yellow oil. LC-MS: (ES+H, m/z) [M+H]⁺=486.20. ¹H NMR (400 MHz, DMSO-d₆) δ 8.52 (s, 1H), 7.29-7.23 (m, 2H), 7.01 (d, J=8.3 Hz, 1H), 6.94-6.83 (m, 4H), 5.02 (s, 2H), 3.69 (d, J=19.4 Hz, 6H), 2.70 (s, 3H), 2.18 (s, 3H), 1.16 (s, 9H)

### Step 6: Preparation of ethyl (S)-3-(((R)-tert-butylsulfinyl)amino)-3-(5-(2-((3,4-dimethoxybenzyl)oxy)-6-methylphenyl)-2-methylthiophen-3-yl)propanoate

A mixture of Zn (865 mg, 13.22 mmol, 5.00 equiv.) and TMSCl (72 mg, 0.66 mmol, 0.25 equiv.) in THF (10 mL) was stirred for 1 h at 60° C. under nitrogen atmosphere. To the above mixture was added ethyl bromoacetate (1.10 g, 6.61 mmol, 2.50 equiv.) dropwise over 2 min at 0° C. The resulting mixture was stirred for an additional 1 h at 60° C. and then (R)—N-[(5-{2-[(3,4-dimethylphenyl)methoxy]-6-methylphenyl}-2-methylthiophen-3-yl)methylidene]-2-methylpropane-2-sulfinamide (1.20 g, 2.65 mmol, 1.00 equiv.) in THF (10 mL) was added dropwise over 20 min at −20° C. The resulting mixture was stirred for an additional 2 h at 0° C. and then diluted with water (50 mL) and extracted with EtOAc (3×150 mL). The combined organic layers were washed with brine (3×50 mL), dried over anhydrous Na₂SO₄, filtered, and concentrated. The residue was purified by silica gel column chromatography (petroleum ether/EtOAc (40% to 100% in 20 min) to afford ethyl (S)-3-(((R)-tert-butylsulfinyl)amino)-3-(5-(2-((3,4-dimethoxybenzyl)oxy)-6-methylphenyl)-2-methylthiophen-3-yl)propanoate (1.00 g, 69.8%) as colorless oil. LC-MS: (ES+H, m/z) [M+Na]⁺=596.2.

### Step 7: Preparation of ethyl (3S)-3-amino-3-[5-(2-hydroxy-6-methylphenyl)-2-methylthiophen-3-yl]propanoate

A solution of ethyl (S)-3-(((R)-tert-butylsulfinyl)amino)-3-(5-(2-((3,4-dimethoxybenzyl)oxy)-6-methylphenyl)-2-methylthiophen-3-yl)propanoate (900 mg, 1.66 mmol, 1.00 equiv.) and HCl (gas) in 1,4-dioxane (5 mL, 4 M) in CH₂Cl₂ (5 mL) was stirred for 2 h at room temperature under nitrogen atmosphere and then concentrated. The residue was purified by reversed-phase flash chromatography (C18 gel; mobile phase, MeCN in water (0.1% NH₃·H₂O), 20% to 50% gradient in 15 min; detector, UV 220 nm) to provide ethyl (3S)-3-amino-3-[5-(2-hydroxy-6-methylphenyl)-2-methylthiophen-3-yl]propanoate (400 mg, 75.4%, ee %=99.5%) as light yellow oil. LC-MS: (ES−H, m/z) [M−H]⁻=317.85. ¹H NMR (400 MHz, CDCl₃) δ 7.14 (t, J=7.9 Hz, 1H), 6.95 (s, 1H), 6.80 (t, J=8.3 Hz, 2H), 4.57 (dd, J=8.4, 5.6 Hz, 1H), 4.17-4.08 (m, 2H), 2.87-2.63 (m, 2H), 2.47 (s, 3H), 2.16 (s, 3H), 2.01 (s, 1H), 1.23 (t, J=7.1 Hz, 3H).

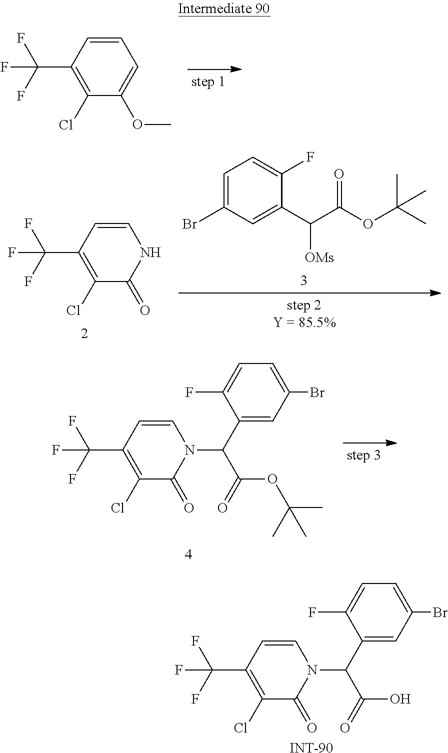

### Step 1: Preparation of 3-chloro-4-(trifluoromethyl)pyridin-2(1H)-one

A solution of 3-chloro-2-methoxy-4-(trifluoromethyl)pyridine (2.00 g, 9.453 mmol, 1.00 equiv.) in HBr in AcOH (33 wt. %. 20 mL) was stirred for 1 h at 60° C. under nitrogen atmosphere. The mixture was allowed to cool down to room temperature. The mixture was basified to pH 9 with saturated NaHCO₃ (aq.). The resulting mixture was extracted with EtOAc (3×50 mL) and the combined organic layers were washed with brine (1×50 mL), dried over anhydrous Na₂SO₄, filtered, and concentrated to provide 3-chloro-4-(trifluoromethyl)pyridin-2(1H)-one (2.00 g, crude) as a white solid. LC-MS: (ES−H, m/z) [M−H]⁻=195.9. ¹H NMR (300 MHz, DMSO-d₆) δ 12.81 (s, 1H), 7.66 (d, 1H), 6.51 (d, 1H). ¹⁹F NMR (282 MHz, DMSO-d₆) δ −61.98.

### Step 2: Preparation of tert-butyl 2-(5-bromo-2-fluorophenyl)-2-(3-chloro-2-oxo-4-(trifluoromethyl)pyridin-1(2H)-yl)acetate

A mixture of 3-chloro-4-(trifluoromethyl)-1H-pyridin-2-one (1.00 g, 5.06 mmol, 1.00 equiv.), tert-butyl 2-(5-bromo-2-fluorophenyl)-2-(methanesulfonyloxy)acetate (2.33 g, 6.07 mmol, 1.20 equiv.) and K₂CO₃ (2.10 g, 15.18 mmol, 3.00 equiv.) in MeCN (40 mL) was stirred for 2 h at 80° C. under nitrogen atmosphere. The mixture cooled to room temperature and filtered. The filter cake was washed with CH₂Cl₂ (2×10 mL) and the combined filtrates were concentrated under reduced pressure. The residue was purified by silica gel column chromatography (PE/EtOAc (0% to 20% gradient in 30 min)) to afford tert-butyl 2-(5-bromo-2-fluorophenyl)-2-(3-chloro-2-oxo-4-(trifluoromethyl)pyridin-1(2H)-yl)acetate (2.10 g, 85.5%) as an off-white solid. LC-MS: (ES−H, m/z) [M−H]⁻=481.7.

### Step 3: Preparation of 2-(5-bromo-2-fluorophenyl)-2-(3-chloro-2-oxo-4-(trifluoromethyl)pyridin-1(2H)-yl)acetic acid

A mixture of tert-butyl 2-(5-bromo-2-fluorophenyl)-2-[3-chloro-2-oxo-4-(trifluoromethyl)pyridin-1-yl]acetate (2.10 g, 4.33 mmol, 1.00 equiv.) and TFA (10 mL, 134.63 mmol, 31.07 equiv.) in CH₂Cl₂ (2 mL) was stirred for 2 h at room temperature under nitrogen atmosphere. The resulting mixture was concentrated under reduced pressure to provide 2-(5-bromo-2-fluorophenyl)-2-(3-chloro-2-oxo-4-(trifluoromethyl)pyridin-1(2H)-yl)acetic acid (1.80 g, crude) as an off-white solid. LC-MS: (ES+H, m/z) [M+H]⁺=427.9.

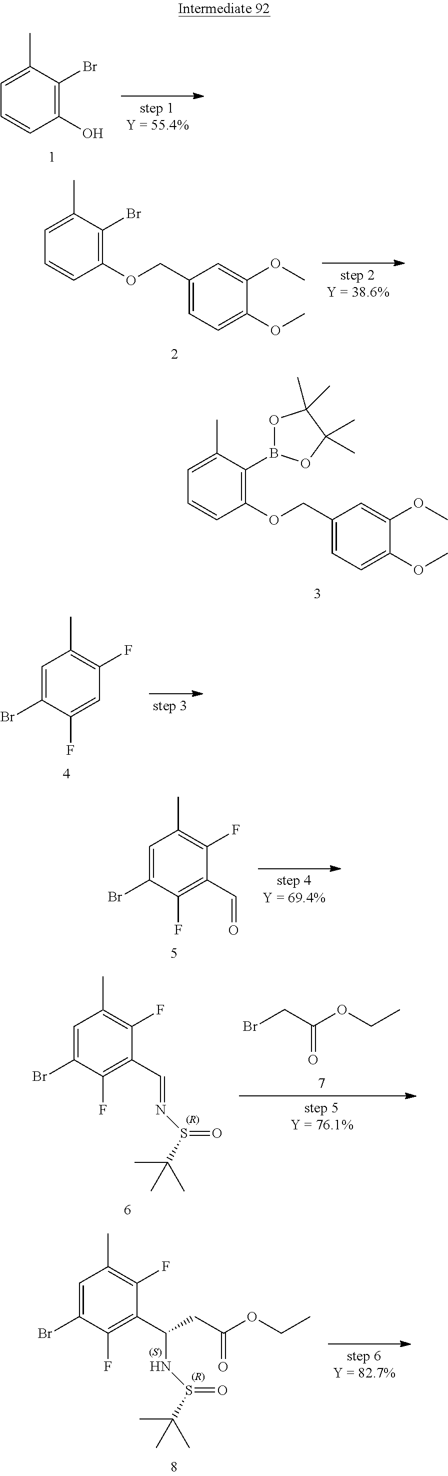

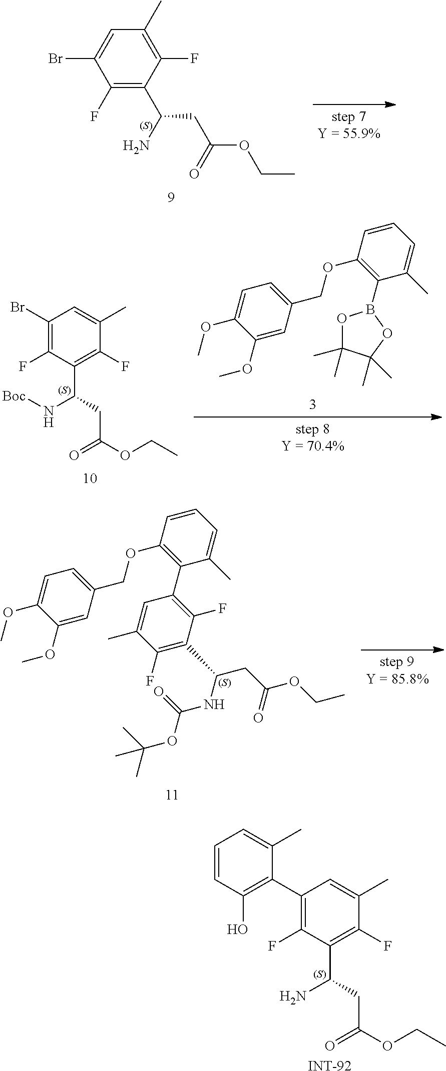

### Step 1: Preparation of 2-bromo-1-[(3,4-dimethoxyphenyl)methoxy]-3-methylbenzene

To a stirred mixture of 2-bromo-3-methylphenol (5.00 g, 26.73 mmol, 1.00 equiv.) and 4-(bromomethyl)-1,2-dimethoxybenzene (9.27 g, 40.09 mmol, 1.50 equiv.) in acetonitrile (50 mL) was added KOH (3.75 g, 66.83 mmol, 2.50 equiv.) in portions at room temperature under nitrogen atmosphere. The resulting mixture was stirred overnight at 60° C. and then cooled to room temperature. The resulting mixture was filtered and the filter cake was washed with EtOAc. The filtrate was concentrated under reduced pressure and the residue was purified by silica gel column chromatography (Ethyl acetate/petroleum ether (0˜20% in 20 min) to afford 2-bromo-1-[(3,4-dimethoxyphenyl)methoxy]-3-methylbenzene (5 g, 55.4%) as a white solid. ¹H NMR (400 MHz, DMSO-d₆) δ 7.21 (t, J=7.9 Hz, 1H), 7.10 (d, J=1.9 Hz, 1H), 7.04-6.91 (m, 4H), 5.10 (s, 2H), 3.79-3.72 (m, 6H), 2.35 (s, 3H).

### Step 2: Preparation of 2-{2-[(3,4-dimethoxyphenyl)methoxy]-6-methylphenyl}-4,4,5,5-tetramethyl-1,3,2-dioxaborolane

To a stirred mixture of 2-bromo-1-[(3,4-dimethoxyphenyl)methoxy]-3-methylbenzene (5.00 g, 14.82 mmol, 1.00 equiv.) and bis(pinacolato)diboron (4.52 g, 17.79 mmol, 1.20 equiv.) in 1,4-dioxane (50 mL) were added Pd(dppf)Cl₂·CH₂Cl₂ (603 mg, 0.74 mmol, 0.05 equiv.) and KOAc (4.35 g, 44.33 mmol, 2.99 equiv.) at room temperature under nitrogen atmosphere. The resulting mixture was stirred overnight at 100° C. under nitrogen atmosphere and then cooled to room temperature. The resulting mixture was filtered and the filter cake was washed with EtOAc. The filtrate was concentrated under reduced pressure and the residue was purified by silica gel column chromatography (Ethyl acetate/petroleum ether (10˜40% in 20 min) to afford 2-{2-[(3,4-dimethoxyphenyl)methoxy]-6-methylphenyl}-4,4,5,5-tetramethyl-1,3,2-dioxaborolane (2.2 g, 38.6%) as a white solid. ¹H NMR (400 MHz, DMSO-d₆) δ 7.21-7.15 (m, 1H), 7.04-6.97 (m, 2H), 6.91 (d, J=8.1 Hz, 1H), 6.81 (d, J=8.3 Hz, 1H), 6.72 (d, J=7.5 Hz, 1H), 4.95 (s, 2H), 3.74 (d, J=4.9 Hz, 6H), 2.23 (s, 3H), 1.23 (s, 12H).

### Step 3: Preparation of 3-bromo-2,6-difluoro-5-methylbenzaldehyde

A solution of 1-bromo-2,4-difluoro-5-methylbenzene (25.00 g, 120.76 mmol, 1.00 equiv.) in THF (150 mL) was treated with LDA (90 mL, 181.18 mmol, 1.50 equiv., 2 M in THF) for 0.5 h at −78° C. under nitrogen atmosphere. The resulting mixture was stirred for 1 h at −78° C. and then DMF (30 mL, 387.64 mmol, 3.21 equiv.) was added dropwise at −78° C. The resulting mixture was stirred for an additional 1 h at −78° C. and then quenched by the addition of 1M HCl (aq.) (150 mL) at 0° C. The resulting mixture was extracted with EtOAc (3×300 mL) and the combined organic layers were washed with brine (3×300 mL), dried over anhydrous Na₂SO₄, filtered and concentrated to afford 3-bromo-2,6-difluoro-5-methylbenzaldehyde (22 g, crude) as a yellow solid. The crude product was used in the next step directly without further purification.

### Step 4: Preparation of (R)—N-[(3-bromo-2,6-difluoro-5-methylphenyl)methylidene]-2-methylpropane-2-sulfinamide

To a stirred mixture of 3-bromo-2,6-difluoro-5-methylbenzaldehyde (5.00 g, 21.27 mmol, 1.00 equiv.) and (R)-2-methylpropane-2-sulfinamide (3.09 g, 25.49 mmol, 1.20 equiv.) in THF (50 mL) was added Ti(Oi-Pr)₄ (12.09 g, 42.54 mmol, 2.00 equiv.) dropwise at room temperature under nitrogen atmosphere. The resulting mixture was stirred for 3 h at room temperature and then quenched by the addition of ice water at room temperature. The resulting mixture was filtered and the filter cake was washed with EtOAc. The filtrate was concentrated under reduced pressure and the residue was purified by silica gel column chromatography (Ethyl acetate/petroleum ether (0˜20% in 20 min) to afford (R)—N-[(3-bromo-2,6-difluoro-5-methylphenyl)methylidene]-2-methylpropane-2-sulfinamide (5 g, 69.4%) as a colorless oil. LC-MS: (ES+H, m/z) [M+H]⁺=338.

### Step 5: Preparation of ethyl (3S)-3-(3-bromo-2,6-difluoro-5-methylphenyl)-3-{[(R)-2-methylpropane-2-sulfinyl]amino}propanoate

A mixture of Zn (4.83 g, 73.92 mmol, 5.00 equiv.) and TMSCl (321 mg, 2.95 mmol, 0.20 equiv.) in THF (50 mL) was stirred for 1 h at 60° C. under nitrogen atmosphere. The mixture was allowed to cool down to room temperature. To the above mixture was added ethyl bromoacetate (6.17 g, 36.96 mmol, 2.50 equiv.) dropwise over 0.5 h at −20° C. The resulting mixture was stirred for an additional 1 h at 50° C. To the above mixture was added (R)—N-[(3-bromo-2,6-difluoro-5-methylphenyl)methylidene]-2-methylpropane-2-sulfinamide (5.00 g, 14.78 mmol, 1.00 equiv.) in THF (20 mL) dropwise over 0.5 h at −20° C. The resulting mixture was stirred for an additional 1 h at room temperature and then filtered. The filter cake was washed with EtOAc and the filtrate was concentrated under reduced pressure. The residue was purified by silica gel column chromatography (Ethyl acetate/petroleum ether (0˜25% in 20 min) to afford ethyl (3S)-3-(3-bromo-2,6-difluoro-5-methylphenyl)-3-{[(R)-2-methylpropane-2-sulfinyl]amino}propanoate (4.8 g, 76.1%) as a yellow oil. LC-MS: (ES+H, m/z) [M+H]⁺=426. ¹H NMR (400 MHz, DMSO-d₆) δ 7.64 (t, 1H), 5.74 (d, 1H), 5.08 (dt, J=8.6, 5.8 Hz, 1H), 4.05-3.95 (m, 2H), 3.14 (dd, J=16.0, 6.5 Hz, 1H), 2.97 (dd, J=16.0, 8.7 Hz, 1H), 2.26-2.14 (m, 3H), 1.14-0.97 (m, 12H). ¹⁹F NMR (377 MHz, DMSO-d₆) δ −109.62, −116.34.

### Step 6: Preparation of ethyl (3S)-3-amino-3-(3-bromo-2,6-difluoro-5-methylphenyl)propanoate

A solution of ethyl (3S)-3-(3-bromo-2,6-difluoro-5-methylphenyl)-3-{[(R)-2-methylpropane-2-sulfinyl]amino}propanoate (4.80 g, 11.29 mmol, 1.00 equiv.) in CH₂Cl₂ (25 mL) was treated with HCl (gas) in 1,4-dioxane (8 mL, 4 M) dropwise over 5 min at room temperature under nitrogen atmosphere. After 1 h the reaction was concentrated and the crude product was purified by reversed-phase flash chromatography (C18 gel; mobile phase, MeCN in water (10 mmol/L NH₄HCO₃), 20% to 50% gradient in 25 min; detector, UV 220 nm) to afford ethyl (3S)-3-amino-3-(3-bromo-2,6-difluoro-5-methylphenyl)propanoate (3 g, 82.7%) as a yellow oil. LC-MS: (ES+H, m/z) [M+H]⁺=322.

### Step 7: Preparation of ethyl (3S)-3-(3-bromo-2,6-difluoro-5-methylphenyl)-3-[(tert-butoxycarbonyl)amino]propanoate

To a stirred mixture of ethyl (3S)-3-amino-3-(3-bromo-2,6-difluoro-5-methylphenyl)propanoate (3.00 g, 9.31 mmol, 1.00 equiv.) and Boc₂O (2.44 g, 11.17 mmol, 1.20 equiv.) in CH₂Cl₂ (30 mL) was added DIEA (3.61 g, 27.93 mmol, 3.00 equiv.) dropwise at room temperature under nitrogen atmosphere. The resulting mixture was stirred for 2 h at room temperature and then concentrated. The residue was purified by silica gel column chromatography (Ethyl acetate/petroleum ether (10˜50% in 25 min) to afford ethyl (3S)-3-(3-bromo-2,6-difluoro-5-methylphenyl)-3-[(tert-butoxycarbonyl)amino]propanoate (2.9 g, ee %=78.9%) as a yellow oil. The product (2.9 μg) was further purified by SFC (Column: CHIRALPAK IG, 3*25 cm, 5 μm; Mobile Phase A: Hexane (10 mM NH₃-MeOH), Mobile Phase B: EtOH; Flow rate: 40 mL/min; Gradient: isocratic 30; wavelength: 219/276 nm; RT1 (min) 4.55; RT2 (min) 7.78; Sample Solvent: EtOH; Injection Volume: 1.8 mL; Number of Runs: 3) to afford 2-{2-[(3,4-dimethoxyphenyl)methoxy]-6-methylphenyl}-4,4,5,5-tetramethyl-1,3,2-dioxaborolane (2.2 g, 55.9%, ee %=100%) as a colorless oil. LC-MS: (ES+H, m/z) [M−tBu]⁺=366. ¹H NMR (400 MHz, DMSO-d₆) δ 7.70-7.52 (m, 2H), 5.34-5.10 (m, 1H), 4.00 (q, 2H), 2.96 (dd, J=16.0, 8.0 Hz, 1H), 2.81 (dd, J=16.1, 7.7 Hz, 1H), 2.25-2.12 (m, 3H), 1.34 (s, 9H), 1.09 (t, J=7.1 Hz, 3H). ¹⁹F NMR (377 MHz, DMSO-d₆) δ −110.74, −117.93.

### Step 8: Preparation of ethyl (3S)-3-[(tert-butoxycarbonyl)amino]-3-{2′-[(3,4-dimethoxyphenyl)methoxy]-2,4-difluoro-5,6′-dimethyl-[1,1′-biphenyl]-3-yl}propanoate

To a stirred mixture of ethyl (3S)-3-(3-bromo-2,6-difluoro-5-methylphenyl)-3-[(tert-butoxycarbonyl)amino]propanoate (1.80 g, 4.26 mmol, 1.00 equiv.) and 2-{2-[(3,4-dimethoxyphenyl)methoxy]-6-methylphenyl}-4,4,5,5-tetramethyl-1,3,2-dioxaborolane (1.97 g, 5.11 mmol, 1.20 equiv.) in 1,4-dioxane (20 mL) were added XPhos Pd G3 (180 mg, 0.21 mmol, 0.05 equiv.) and TBAF (2.79 g, 10.65 mmol, 2.50 equiv.) in portions at room temperature under nitrogen atmosphere. The resulting mixture was stirred for 3 h at 100° C. and then cooled to room temperature and filtered. The filter cake was washed with EtOAc and the combined filtrates were concentrated under reduced pressure. The residue was purified by silica gel column chromatography (Ethyl acetate/petroleum ether (20˜60% in 25 min) to afford ethyl (3S)-3-[(tert-butoxycarbonyl)amino]-3-{2′-[(3,4-dimethoxyphenyl)methoxy]-2,4-difluoro-5,6′-dimethyl-[1,1′-biphenyl]-3-yl}propanoate (1.8 g, 70.4%) as a yellow solid. ¹H NMR (400 MHz, DMSO-d₆) δ 7.59 (q, J=7.8, 6.9 Hz, 1H), 7.18 (t, J=7.9 Hz, 1H), 7.06-6.96 (m, 2H), 6.91 (d, J=8.1 Hz, 1H), 6.81 (d, J=8.3 Hz, 1H), 6.72 (d, J=7.5 Hz, 1H), 5.76 (s, 1H), 4.95 (s, 2H), 4.08-3.95 (m, 2H), 3.74 (d, J=5.0 Hz, 6H), 3.01-2.73 (m, 2H), 2.23 (s, 3H), 1.29-1.18 (m, 12H), 1.09 (t, J=7.1 Hz, 3H).

### Step 9: Preparation of ethyl (3S)-3-amino-3-{2,4-difluoro-2′-hydroxy-5,6′-dimethyl-[1,1′-biphenyl]-3-yl}propanoate

A solution of ethyl (3S)-3-[(tert-butoxycarbonyl)amino]-3-{2′-[(3,4-dimethoxyphenyl)methoxy]-2,4-difluoro-5,6′-dimethyl-[1,1′-biphenyl]-3-yl}propanoate (1.80 g, 3.00 mmol, 1.00 equiv.) in CH₂Cl₂ (20 mL) was treated with HCl (gas) in 1,4-dioxane (8 mL, 4 M) dropwise over 5 min at room temperature under nitrogen atmosphere. After 1 h, the reaction was concentrated and the residue was purified by reversed-phase flash chromatography (C18 gel; mobile phase, MeCN in water, 20% to 50% gradient in 25 min; detector, UV 220 nm) to afford (3S)-3-amino-3-{2,4-difluoro-2′-hydroxy-5,6′-dimethyl-[1,1′-biphenyl]-3-yl}propanoate (900 mg, 85.8%) as a yellow oil. LC-MS: (ES+H, m/z) [M+H]⁺=350.1. ¹H NMR (400 MHz, DMSO-d₆) δ 9.28 (s, 1H), 7.07 (t, J=7.8 Hz, 1H), 6.99 (t, J=8.4 Hz, 1H), 6.79-6.69 (m, 2H), 4.61-4.52 (m, 1H), 4.04-3.93 (m, 2H), 2.88-2.64 (m, 2H), 2.21 (s, 3H), 1.95 (d, J=3.8 Hz, 3H), 1.17-1.01 (m, 3H). ¹⁹F NMR (377 MHz, DMSO-d₆) δ −119.09, −120.23.

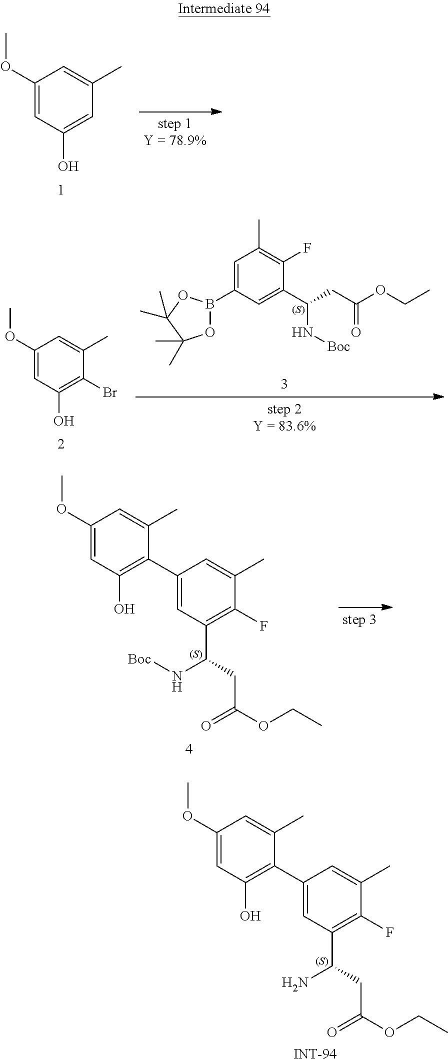

### Step 1: Preparation of 2-bromo-5-methoxy-3-methylphenol

To a stirred mixture of 5-methoxy-m-cresol (5.00 g, 36.19 mmol, 1.00 equiv.) in CCl₄ (100 mL) was added NBS (5.80 g, 32.57 mmol, 0.90 equiv.) in portions at 0° C. The resulting mixture was stirred for 2 h at room temperature. The resulting mixture was filtered and the filter cake was washed with EtOAc (3×50 mL). The filtrate was concentrated under reduced pressure and the residue was purified by silica gel column chromatography (PE/EtOAc (0% to 30% gradient in 20 min) to afford 2-bromo-5-methoxy-3-methylphenol (6.2 g, 78.9%) as a white solid. LC-MS: (ES−H, m/z) [M−H]⁻=214.85. ¹H NMR (400 MHz, CDCl₃) δ 6.46 (d, J=2.9 Hz, 1H), 6.42 (d, J=2.9 Hz, 1H), 5.58 (s, 1H), 3.76 (s, 3H), 2.36 (s, 3H).

### Step 2: Preparation of ethyl (3S)-3-[(tert-butoxycarbonyl)amino]-3-{4-fluoro-2′-hydroxy-4′-methoxy-5,6′-dimethyl-[1,1′-biphenyl]-3-yl}propanoate

To a stirred solution of 2-bromo-5-methoxy-3-methylphenol (450 mg, 2.07 mmol, 1.00 equiv.) and ethyl (3S)-3-[(tert-butoxycarbonyl)amino]-3-[2-fluoro-3-methyl-5-(4,4,5,5-tetramethyl-1,3,2-dioxaborolan-2-yl)phenyl]propanoate (1029 mg, 2.28 mmol, 1.10 equiv.) in 1,4-dioxane/H₂O=10:1 (15 mL) were added Pd(dppf)Cl₂ (75 mg, 0.10 mmol, 0.05 equiv.) and K₂CO₃ (716 mg, 5.18 mmol, 2.50 equiv.) at room temperature. The resulting mixture was stirred for 2 h at 100° C. under nitrogen atmosphere and then cooled to room temperature. The resulting mixture was diluted with water (50 mL), and extracted with EtOAc (3×50 mL). The combined organic layers were washed with brine (2×20 mL), dried over anhydrous Na₂SO₄, filtered, and concentrated. The residue was purified by silica gel column chromatography (petroleum ether/EtOAc (20% to 60% in 20 min) to afford ethyl (3S)-3-[(tert-butoxycarbonyl)amino]-3-{4-fluoro-2′-hydroxy-4′-methoxy-5,6′-dimethyl-[1,1′-biphenyl]-3-yl}propanoate (800 mg, 83.6%) as a yellow solid. LC-MS: (ES−H, m/z) [M−H]⁻=460.20. ¹H NMR (400 MHz, DMSO-d₆) δ 9.13 (s, 1H), 7.50 (d, J=9.0 Hz, 1H), 7.19 (dt, J=29.0, 7.4 Hz, 1H), 7.05 (t, J=7.5 Hz, 1H), 7.00 (d, J=6.6 Hz, 1H), 6.95-6.88 (m, 1H), 5.31-5.23 (m, 1H), 4.05 (qd, J=7.1, 3.9 Hz, 2H), 3.69 (s, 3H), 2.66-2.61 (m, 2H), 2.24-2.22 (m, 3H), 1.92 (s, 3H), 1.34 (d, J=4.4 Hz, 9H), 1.14 (dd, J=7.1, 3.1 Hz, 3H).

### Step 3: Preparation of ethyl (3S)-3-amino-3-{4-fluoro-2′-hydroxy-4′-methoxy-5,6′-dimethyl-[1,1′-biphenyl]-3-yl}propanoate

To a stirred mixture of ethyl (3S)-3-[(tert-butoxycarbonyl)amino]-3-{4-fluoro-2′-hydroxy-4′-methoxy-5,6′-dimethyl-[1,1′-biphenyl]-3-yl}propanoate (770 mg, 1.668 mmol, 1 equiv.) in CH₂C₂ (10 mL) was added HCl (gas) in 1,4-dioxane (5 mL, 4 M) at room temperature under nitrogen atmosphere. The resulting mixture was stirred for 1 h at room temperature and then concentrated to provide ethyl (3S)-3-amino-3-{4-fluoro-2′-hydroxy-4′-methoxy-5,6′-dimethyl-[1,1′-biphenyl]-3-yl}propanoate (600 mg, crude) as a yellow solid. LC-MS: (ES+H, m/z) [M+H]⁺=362.05.

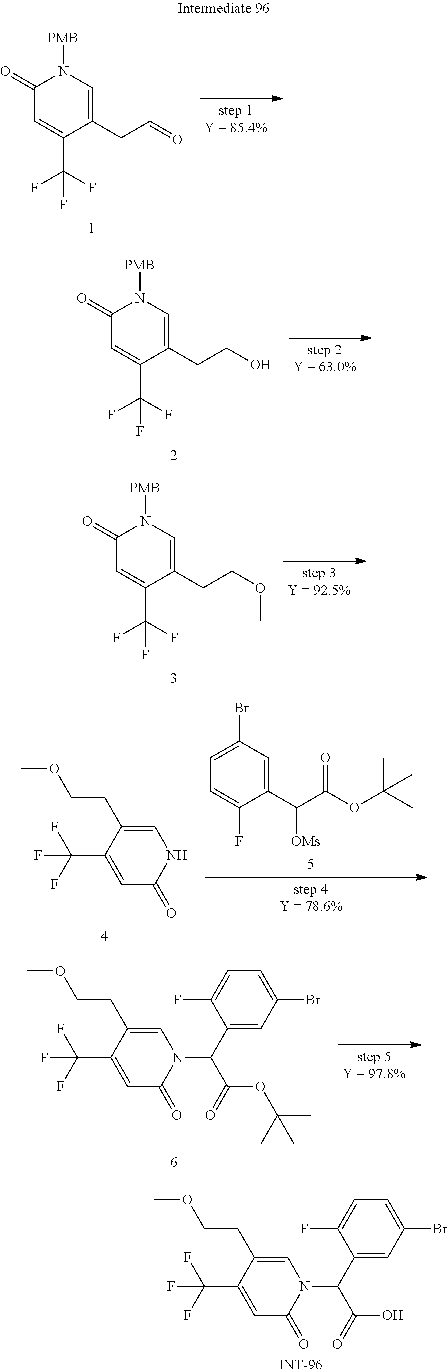

### Step 1: Preparation of 5-(2-hydroxyethyl)-1-[(4-methoxyphenyl)methyl]-4-(trifluoromethyl)pyridin-2-one

A solution of 2-{1-[(4-methoxyphenyl)methyl]-6-oxo-4-(trifluoromethyl)pyridin-3-yl}acetaldehyde (5.00 g, 15.37 mmol, 1.00 equiv.) and NaBH₄ (1.16 g, 30.74 mmol, 2.00 equiv.) in MeOH (60 mL) was stirred at −15° C. under nitrogen atmosphere. After 2 h, the resulting mixture was concentrated under reduced pressure and the residue was purified by silica gel column chromatography (petroleum ether/EtOAc (0 to 60% gradient in 20 min) to afford 5-(2-hydroxyethyl)-1-[(4-methoxyphenyl) methyl]-4-(trifluoromethyl) pyridin-2-one (4.3 g, 85.4%) as a yellow oil. LC-MS: (ES−H, m/z) [M+H]⁺=328.1. ¹H NMR (400 MHz, CD₃OD) δ 7.77 (s, 1H), 7.35-7.26 (m, 2H), 6.94-6.86 (m, 2H), 6.84 (s, 1H), 5.13 (s, 2H), 3.77 (s, 3H), 3.66 (t, J=6.6 Hz, 2H), 2.72 (t, J=6.6 Hz, 2H).

### Step 2: Preparation of 5-(2-methoxyethyl)-1-[(4-methoxyphenyl)methyl]-4-(trifluoromethyl)pyridin-2-one

To a stirred solution of 5-(2-hydroxyethyl)-1-[(4-methoxyphenyl)methyl]-4-(trifluoromethyl)pyridin-2-one (3.80 g, 11.61 mmol, 1.00 equiv.) and NaH (557 mg, 13.93 mmol, 1.20 equiv., 60%) in DMF (45 mL) was added CH₃I (2.47 g, 17.42 mmol, 1.50 equiv.) in portions at −15° C. The resulting mixture was stirred for 3 h at room temperature and then quenched by the addition of ice water (100 mL) at room temperature. The resulting mixture was extracted with EtOAc (3×50 mL) and the combined organic layers were washed with brine (1×40 mL), dried over anhydrous Na₂SO₄, filtered, and concentrated. The residue was purified by silica gel column chromatography (petroleum ether/EtOAc (0 to 60% gradient in 20 min) to afford 5-(2-methoxyethyl)-1-[(4-methoxyphenyl)methyl]-4-(trifluoromethyl)pyridin-2-one (2.5 g, 63.0%) as a yellow solid. LC-MS: (ES+H, m/z) [M+H]⁺=342.0. ¹H NMR (400 MHz, CD₃OD) δ 7.75 (s, 1H), 7.34-7.26 (m, 2H), 6.94-6.86 (m, 2H), 6.83 (s, 1H), 5.13 (s, 2H), 3.77 (s, 3H), 3.49 (t, J=6.3 Hz, 2H), 3.28 (s, 3H), 2.75 (t, J=6.3 Hz, 2H).

### Step 3: Preparation of 5-(2-methoxyethyl)-4-(trifluoromethyl)-1H-pyridin-2-one

A mixture of 5-(2-methoxyethyl)-1-[(4-methoxyphenyl)methyl]-4-(trifluoromethyl)pyridin-2-one (2.50 g, 7.32 mmol, 1.00 equiv.) in CF₃COOH (30 mL) was stirred overnight at 80° C. under nitrogen atmosphere. The resulting mixture was concentrated under reduced pressure and the residue was purified by reversed-phase flash chromatography (C18 gel; mobile phase, MeCN in water (10 mmol/L NH₄HCO₃), 10% to 60% gradient in 20 min; detector, UV 220 nm) to provide 5-(2-methoxyethyl)-4-(trifluoromethyl)-1H-pyridin-2-one (1.5 g, 92.5%) as a white solid. LC-MS: (ES−H, m/z) [M−H]⁻=220.0. ¹H NMR (400 MHz, CD₃OD) δ 7.49 (s, 1H), 6.82 (s, 1H), 3.54 (t, J=6.4 Hz, 2H), 3.34 (s, 3H), 2.78 (t, J=6.4, 1.0 Hz, 2H).

### Step 4: Preparation of tert-butyl 2-(5-bromo-2-fluorophenyl)-2-[5-(2-methoxyethyl)-2-oxo-4-(trifluoromethyl)pyridin-1-yl]acetate

To a stirred solution of 5-(2-methoxyethyl)-4-(trifluoromethyl)-1H-pyridin-2-one (470 mg, 2.13 mmol, 1.00 equiv.) and tert-butyl 2-(5-bromo-2-fluorophenyl)-2-(methanesulfonyloxy)acetate (977 mg, 2.55 mmol, 1.20 equiv.) in MeCN (15 mL) was added K₂CO₃ (587 mg, 4.25 mmol, 2.00 equiv.) in portions at room temperature. The resulting mixture was stirred overnight at 60° C. under nitrogen atmosphere. The resulting mixture was filtered and the filter cake was washed with EtOAc (3×30 mL). The filtrate was concentrated under reduced pressure. The residue was purified by silica gel column chromatography (petroleum ether/EtOAc (0 to 60% gradient in 20 min) to afford tert-butyl 2-(5-bromo-2-fluorophenyl)-2-[5-(2-methoxyethyl)-2-oxo-4-(trifluoromethyl)pyridin-1-yl]acetate (850 mg, 78.6%) as a white solid. LC-MS: (ES−H, m/z) [M−H]⁻=506.1. ¹H NMR (400 MHz, DMSO-d₆) δ 7.77-7.69 (m, 1H), 7.63 (dd, J=6.5, 2.5 Hz, 1H), 7.55 (s, 1H), 7.36 (t, J=10.1, 8.8 Hz, 1H), 6.89 (s, 1H), 6.53 (s, 1H), 3.46-3.34 (m, 2H), 3.15 (s, 3H), 2.65 (t, J=6.2 Hz, 2H), 1.41 (s, 9H).

### Step 5: Preparation of (5-bromo-2-fluorophenyl)[5-(2-methoxyethyl)-2-oxo-4-(trifluoromethyl)pyridin-1-yl]acetic acid

A solution of tert-butyl 2-(5-bromo-2-fluorophenyl)-2-[5-(2-methoxyethyl)-2-oxo-4-(trifluoromethyl)pyridin-1-yl]acetate (850 mg, 1.67 mmol, 1 equiv.) and TFA (5 mL) in DCM (9 mL) was stirred for 2 h at room temperature and then concentrated. The residue was purified by reversed-phase flash chromatography (C18 gel; mobile phase, MeCN in water (10 mmol/L NH₄HCO₃), 0% to 60% gradient in 20 min; detector, UV 220 nm) to provide (5-bromo-2-fluorophenyl)[5-(2-methoxyethyl)-2-oxo-4-(trifluoromethyl)pyridin-1-yl]acetic acid (740 mg, 97.8%) as a white solid. LC-MS: (ES+H, m/z) [M+H]⁺=452.1.

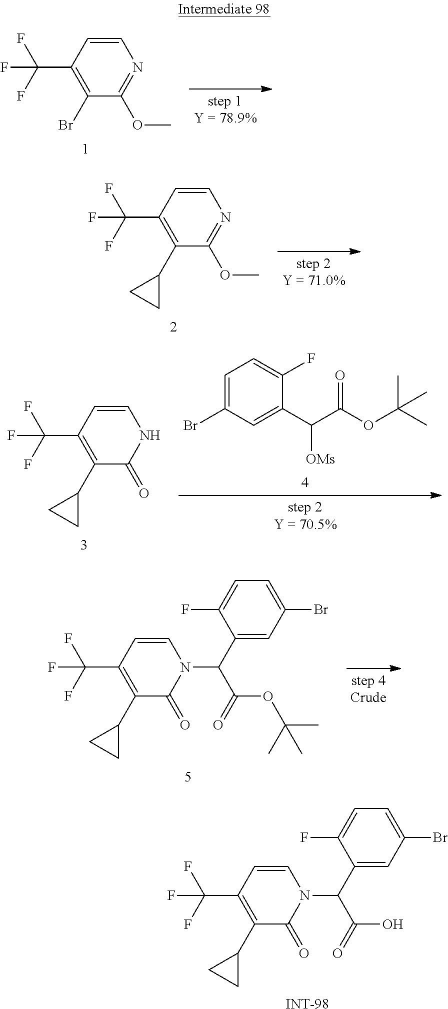

### Step 1: Preparation of 3-cyclopropyl-2-methoxy-4-(trifluoromethyl)pyridine

To a stirred solution of 3-bromo-2-methoxy-4-(trifluoromethyl)pyridine (1.20 g, 4.69 mmol, 1.00 equiv.) and cyclopropylboronic acid (0.60 g, 7.03 mmol, 1.50 equiv.) in toluene (15 mL) and H₂O (3 mL) were added Pd(OAc)₂ (0.11 g, 0.47 mmol, 0.10 equiv.), PCy3 (0.26 g, 0.94 mmol, 0.20 equiv.), and Cs₂CO₃ (3.05 g, 9.36 mmol, 2.00 equiv.) at room temperature under nitrogen atmosphere. The resulting mixture was stirred overnight at 100° C. under nitrogen atmosphere. The mixture was allowed to cool down to room temperature and concentrated. The resulting mixture was diluted with EtOAc (100 mL), washed with brine (3×30 mL), dried over anhydrous Na₂SO₄, filtered, and concentrated. The residue was purified by silica gel column chromatography (petroleum ether/CH₂Cl₂ (20% to 30% gradient in 10 min) to afford 3-cyclopropyl-2-methoxy-4-(trifluoromethyl)pyridine (815 mg, 78.9%) as a colorless oil. LC-MS: (ES+H, m/z) [M+H]⁺=218.10. ¹H NMR (400 MHz, DMSO-d₆) δ 8.26 (d, J=5.3 Hz, 1H), 7.28 (d, J=5.3 Hz, 1H), 3.94 (s, 3H), 1.85 (qd, J=7.4, 6.7, 3.5 Hz, 1H), 1.00 (d, J=7.3 Hz, 4H). ¹⁹F NMR (377 MHz, DMSO-d₆) δ −59.24.

### Step 2: Preparation of 3-cyclopropyl-4-(trifluoromethyl)-1H-pyridin-2-one

A solution of 3-cyclopropyl-2-methoxy-4-(trifluoromethyl)pyridine (800 mg, 3.68 mmol, 1.00 equiv.) and TMSI (2.94 g, 14.73 mmol, 4.00 equiv.) in MeCN (10 mL) was stirred overnight at 60° C. under nitrogen atmosphere. The mixture was allowed to cool down to room temperature and concentrated under reduced pressure. The resulting mixture was diluted with EtOAc (100 mL), washed with brine (3×30 mL), dried over anhydrous Na₂SO₄, filtered, and concentrated. The residue was purified by silica gel column chromatography (petroleum ether/EtOAc (10% to 20% gradient in 10 min) to afford 3-cyclopropyl-4-(trifluoromethyl)-1H-pyridin-2-one (537 mg, 71.0%) as a white solid. LC-MS: (ES+H, m/z) [M+H]⁺=204.05. ¹H NMR (400 MHz, DMSO-d₆) δ 11.95 (s, 1H), 7.38 (d, J=6.9 Hz, 1H), 6.33 (d, J=6.9 Hz, 1H), 1.76 (ddd, J=8.0, 5.5, 2.6 Hz, 1H), 1.46 (dt, J=6.0, 3.0 Hz, 2H), 0.78 (dt, J=8.7, 3.1 Hz, 2H). ¹⁹F NMR (377 MHz, DMSO-d₆) δ −59.31.

### Step 3: Preparation of tert-butyl 2-(5-bromo-2-fluorophenyl)-2-[3-cyclopropyl-2-oxo-4-(trifluoromethyl)pyridin-1-yl]acetate

To a stirred solution of 3-cyclopropyl-4-(trifluoromethyl)-1H-pyridin-2-one (517 mg, 2.55 mmol, 1.00 equiv.) and tert-butyl 2-(5-bromo-2-fluorophenyl)-2-(methanesulfonyloxy)acetate (1.07 g, 2.80 mmol, 1.10 equiv.) in MeCN (10 mL) was added K₂CO₃ (1.06 g, 7.64 mmol, 3.00 equiv.) at room temperature under nitrogen atmosphere. The resulting mixture was stirred overnight at 80° C. under nitrogen atmosphere and then cooled to room temperature. The resulting mixture was concentrated under reduced pressure, diluted with EtOAc (100 mL), washed with brine (3×30 mL), dried over anhydrous Na₂SO₄, filtered, and concentrated. The residue was purified by reversed-phase flash chromatography (C18 gel; mobile phase, MeCN in water (0.11% NH₃·H₂O), 90% to 100% gradient in 10 min; detector, UV 254 nm) to afford tert-butyl 2-(5-bromo-2-fluorophenyl)-2-[3-cyclopropyl-2-oxo-4-(trifluoromethyl)pyridin-1-yl]acetate (879 mg, 70.5%) as a yellow solid. LC-MS: (ES+H, m/z) [M+H]⁺=490.00. ¹H NMR (300 MHz, DMSO-d₆) δ 8.23 (d, J=5.3 Hz, 1H), 7.80 (dd, J=6.2, 2.6 Hz, 1H), 7.71 (ddd, J=8.8, 4.6, 2.6 Hz, 1H), 7.43-7.32 (m, 2H), 6.41 (s, 1H), 1.31 (s, 9H), 1.26 (s, 1H), 1.14-1.02 (m, 2H), 1.02-0.91 (m, 2H). ¹⁹F NMR (282 MHz, DMSO-d₆) δ −59.81, −116.45.

### Step 4: Preparation of (5-bromo-2-fluorophenyl)[3-cyclopropyl-2-oxo-4-(trifluoromethyl)pyridin-1-yl]acetic acid

A solution of tert-butyl 2-(5-bromo-2-fluorophenyl)-2-[3-cyclopropyl-2-oxo-4-(trifluoromethyl)pyridin-1-yl]acetate (869 mg, 1.77 mmol, 1.00 equiv.) and CF₃COOH (3 mL) in CH₂Cl₂ (3 mL) was stirred for 2 h at room temperature under nitrogen atmosphere. The resulting mixture was concentrated under reduced pressure to afford (5-bromo-2-fluorophenyl)[3-cyclopropyl-2-oxo-4-(trifluoromethyl)pyridin-1-yl]acetic acid (1.12 g, crude) as a yellow oil. LC-MS: (ES+H, m/z) [M+H]⁺=433.90.

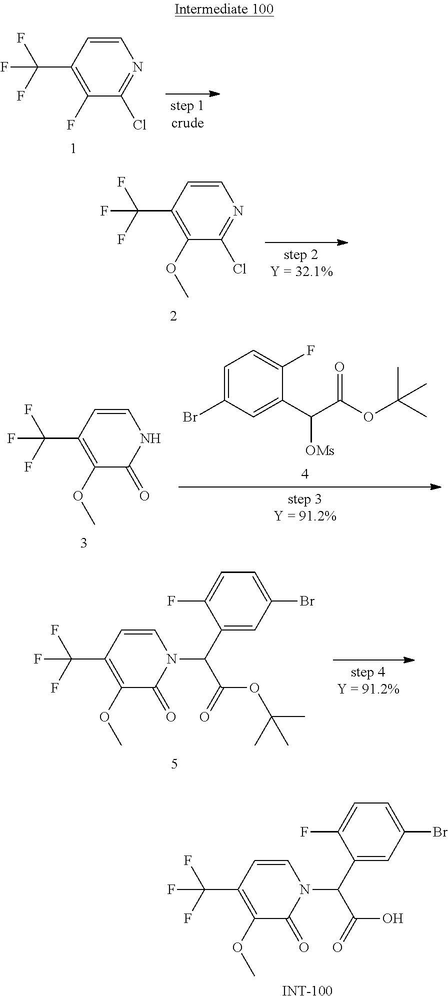

### Step 1: Preparation of 2-chloro-3-methoxy-4-(trifluoromethyl)pyridine

To a stirred solution of 2-chloro-3-fluoro-4-(trifluoromethyl)pyridine (2.00 g, 10.02 mmol, 1.00 equiv.) in CH₃OH (15 mL) was added CH₃ONa (812 mg, 15.03 mmol, 1.50 equiv.) in portions at room temperature under nitrogen atmosphere. The resulting mixture was stirred overnight at 80° C. under nitrogen atmosphere. The mixture was allowed to cool down to room temperature and diluted with water (50 mL). The resulting mixture was extracted with CH₂Cl₂ (3×100 mL) and the combined organic layers were dried over anhydrous Na₂SO₄, filtered, and concentrated at 0° C. to afford 2-chloro-3-methoxy-4-(trifluoromethyl)pyridine (1.7 g, crude) as a colorless oil. The crude product was used in the next step directly without further purification. ¹H NMR (400 MHz, DMSO-d₆) δ 8.45 (dd, J=5.0, 0.9 Hz, 1H), 7.78 (d, J=5.0 Hz, 1H), 3.96 (s, 3H). ¹⁹F NMR (400 MHz, DMSO-d₆) δ −61.80.

### Step 2: Preparation of 3-methoxy-4-(trifluoromethyl)-1H-pyridin-2-one

To a stirred solution of 2-chloro-3-methoxy-4-(trifluoromethyl)pyridine (1.50 g, 7.09 mmol, 1.00 equiv., crude) in t-BuOH (15 mL) was added NaOH (1.10 g, 28.36 mmol, 4.00 equiv.) in portions at room temperature under nitrogen atmosphere. The reaction mixture was stirred for 24 h at 80° C. under nitrogen atmosphere and then cooled to room temperature. The resulting mixture was diluted with water (50 mL), acidified to pH 5˜6 with 1 N HCl (aq.), and extracted with CH₂Cl₂ (5×100 mL). The combined organic layers were dried over anhydrous Na₂SO₄, filtered, and concentrated. The residue was purified by reversed-phase flash chromatography (C18 gel; mobile phase, CH₃CN in water (0.1% HCOOH), 15% to 30% gradient in 20 min; detector, UV 254 nm) to afford 3-methoxy-4-(trifluoromethyl)-1H-pyridin-2-one (440 mg, 32.1%) as a white solid. LC-MS: (ES+H, m/z) [M+H]⁺=194.00. ¹H NMR (400 MHz, DMSO-d₆) δ 12.36 (s, 1H), 7.35-7.30 (m, 1H), 6.29 (d, J=6.9 Hz, 1H), 3.91 (s, 3H). ¹⁹F NMR (400 MHz, DMSO-d₆) δ −61.70.

### Step 3: Preparation of tert-butyl 2-(5-bromo-2-fluorophenyl)-2-[3-methoxy-2-oxo-4-(trifluoromethyl)pyridin-1-yl]acetate

To a stirred solution of 3-methoxy-4-(trifluoromethyl)-1H-pyridin-2-one (390 mg, 2.01 mmol, 1.00 equiv.) and tert-butyl 2-(5-bromo-2-fluorophenyl)-2-(methanesulfonyloxy)acetate (773 mg, 2.01 mmol, 1.00 equiv.) in CH₃CN (50 mL) was added K₂CO₃ (558 mg, 4.03 mmol, 2.00 equiv.) in portions at room temperature under nitrogen atmosphere. The resulting mixture was stirred for 2 h at 80° C. under nitrogen atmosphere and then cooled to room temperature. The resulting mixture was diluted with water (100 mL) and extracted with CH₂Cl₂ (3×200 mL). The combined organic layers were dried over anhydrous Na₂SO₄, filtered, and concentrated. The residue was purified by silica gel column chromatography (PE/EtOAc (8%˜20% in 20 min) to afford tert-butyl 2-(5-bromo-2-fluorophenyl)-2-[3-methoxy-2-oxo-4-(trifluoromethyl)pyridin-1-yl]acetate (885 mg, 91.2%) as a colorless liquid. LC-MS: (ES+H, m/z) [M+H]⁺=480.05. ¹H NMR (400 MHz, DMSO-d₆) δ 7.63-7.59 (m, 1H), 7.56 (dd, J=6.5, 2.5 Hz, 1H), 7.35 (d, J=7.4 Hz, 1H), 7.24 (dd, J=10.1, 8.8 Hz, 1H), 6.47 (s, 1H), 6.33 (d, J=7.4 Hz, 1H), 3.83 (s, 3H), 1.31 (s, 9H). ¹⁹F NMR (400 MHz, DMSO-d₆) δ −61.98, −116.33.

### Step 4: Preparation of (5-bromo-2-fluorophenyl)[3-methoxy-2-oxo-4-(trifluoromethyl)pyridin-1-yl]acetic acid

To a stirred solution of tert-butyl 2-(5-bromo-2-fluorophenyl)-2-[3-methoxy-2-oxo-4-(trifluoromethyl)pyridin-1-yl]acetate (830 mg, 1.72 mmol, 1.00 equiv.) in CH₂Cl₂ (10 mL) was added CF₃COOH (10 mL) dropwise at 0° C. under nitrogen atmosphere. The resulting mixture was stirred for 2 h at room temperature under nitrogen atmosphere and then diluted with CH₂Cl₂ (20 mL) and concentrated. The residue was purified by reversed-phase flash chromatography (C18 gel; mobile phase, CH₃CN in water, 0% to 100% gradient in 15 min; detector, UV 254 nm) to afford (5-bromo-2-fluorophenyl)[3-methoxy-2-oxo-4-(trifluoromethyl)pyridin-1-yl]acetic acid (669 mg, 91.2%) as an off-white solid. LC-MS: (ES+H, m/z) [M+H]⁺=423.95. ¹H NMR (400 MHz, DMSO-d₆) δ 14.00 (s, 1H), 7.73-7.69 (m, 2H), 7.48 (d, J=7.4 Hz, 1H), 7.37-7.25 (m, 1H), 6.63 (s, 1H), 6.42 (d, J=7.5 Hz, 1H), 3.92 (s, 3H). ¹⁹F NMR (400 MHz, DMSO-d₆) δ −61.94, −116.43.

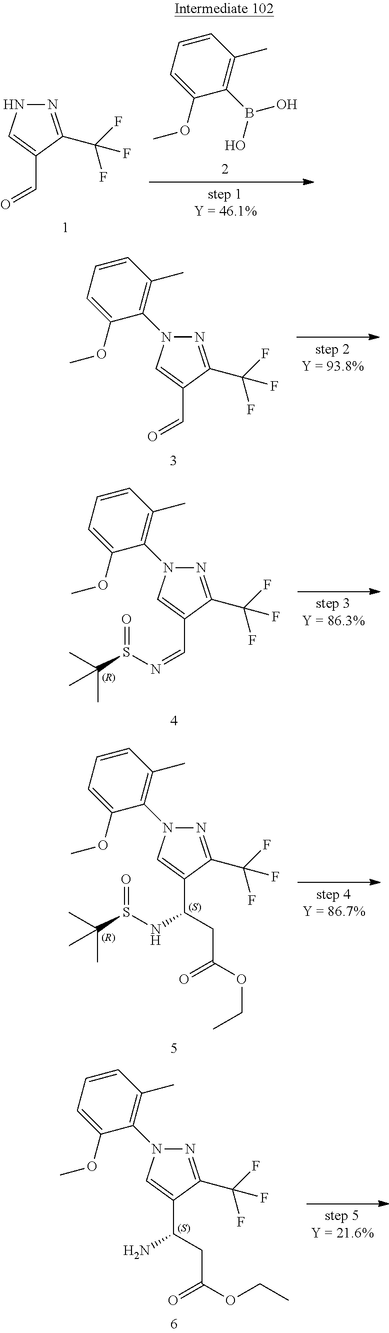

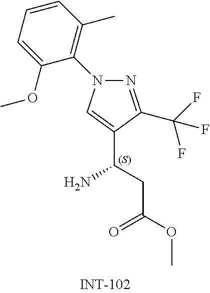

### Step 1: Preparation of 1-(2-methoxy-6-methylphenyl)-3-(trifluoromethyl)pyrazole-4-carbaldehyde

A mixture of 3-(trifluoromethyl)-1H-pyrazole-4-carbaldehyde (2.00 g, 12.19 mmol, 1.00 equiv.), 2-methoxy-6-methylphenylboronic acid (4.05 g, 24.37 mmol, 2.00 equiv.) and Cu₂O (0.87 g, 6.09 mmol, 0.50 equiv.) in MeOH (40 mL) was stirred for 20 h at 50° C. and then cooled to room temperature and concentrated. The residue was purified by silica gel column chromatography (PE/EtOAc (0% to 20% in 20 min) to afford 1-(2-methoxy-6-methylphenyl)-3-(trifluoromethyl)pyrazole-4-carbaldehyde (1.60 g, 46.1%) as a white solid. LC-MS: (ES+H, m/z): [M+H]⁺=285.1. ¹H NMR (300 MHz, DMSO-d₆) δ 9.96 (d, J=0.8 Hz, 1H), 8.93 (q, J=1.0 Hz, 1H), 7.48 (t, J=8.0 Hz, 1H), 7.13 (d, J=8.4 Hz, 1H), 7.07-6.98 (m, 1H), 3.77 (s, 3H), 2.01 (s, 3H).

### Step 2: Preparation of (R)—N-{[1-(2-methoxy-6-methylphenyl)-3-(trifluoromethyl)pyrazol-4-yl]methylidene}-2-methylpropane-2-sulfinamide

A mixture of 1-(2-methoxy-6-methylphenyl)-3-(trifluoromethyl)pyrazole-4-carbaldehyde (1.17 g, 4.12 mmol, 1.00 equiv.), (R)-2-methylpropane-2-sulfinamide (1.00 g, 8.25 mmol, 2.00 equiv.) and tetrakis(propan-2-yloxy)titanium (4.69 g, 16.50 mmol, 4.00 equiv.) in THF (25 mL) was stirred for 16 h at 25° C. under nitrogen atmosphere. The reaction was quenched with water at 25° C. and extracted with EtOAc (3×100 mL). The combined organic layers were washed with brine (1×150 mL), dried over anhydrous Na₂SO₄, filtered and concentrated. The residue was purified by silica gel column chromatography (PE/EtOAc (0% to 30% in 20 min) to afford (R)—N-{[1-(2-methoxy-6-methylphenyl)-3-(trifluoromethyl)pyrazol-4-yl]methylidene}-2-methylpropane-2-sulfinamide (1.5 g, 93.8%) as a yellow solid. LC-MS: (ES+H, m/z): [M+H]⁺=388.2. ¹H NMR (300 MHz, DMSO-d₆) δ 8.89 (d, J=1.1 Hz, 1H), 8.58 (s, 1H), 7.52 (dd, J=8.4, 7.7 Hz, 1H), 7.23-7.02 (m, 2H), 3.81 (s, 3H), 2.06 (s, 3H), 1.23 (s, 9H).

### Step 3: Preparation of ethyl (3S)-3-[1-(2-methoxy-6-methylphenyl)-3-(trifluoromethyl)pyrazol-4-yl]-3-{[(R)-2-methylpropane-2-sulfinyl]amino}propanoate

A mixture of Zn (1.01 g, 15.48 mmol, 4.00 equiv.) in THF (8 mL) was treated with TMSCl (168 mg, 1.54 mmol, 0.40 equiv.) for 1 h at 50° C. under nitrogen atmosphere followed by the addition of 2-bromoethyl acetate (1.62 g, 9.68 mmol, 2.50 equiv.) dropwise at 25° C. and then heated to 50° C. under a nitrogen atmosphere. After 1 h, (R)—N-{[1-(2-methoxy-6-methylphenyl)-3-(trifluoromethyl)pyrazol-4-yl]methylidene}-2-methylpropane-2-sulfinamide (1.50 g, 3.87 mmol, 1.00 equiv.) and THF (4 mL) were added dropwise at 0° C. under nitrogen atmosphere and the reaction was warmed to 25° C. After 1 h, the reaction was quenched with water at 25° C. The resulting mixture was extracted with EtOAc (3×100 mL) and the combined organic layers were washed with brine (1×150 mL), dried over anhydrous Na₂SO₄, filtered, and concentrated. The residue was purified by silica gel column chromatography (PE/EtOAc (0% to 50% in 20 min) to afford ethyl (3S)-3-[1-(2-methoxy-6-methylphenyl)-3-(trifluoromethyl)pyrazol-4-yl]-3-{[(R)-2-methylpropane-2-sulfinyl]amino}propanoate (1.59 g, 86.3%) as a yellow solid. LC-MS: (ES+H, m/z): [M+H]⁺=476.2. ¹H NMR (300 MHz, DMSO-d₆) δ 8.02 (d, J=1.2 Hz, 1H), 7.43 (t, J=8.0 Hz, 1H), 7.08 (d, J=8.3 Hz, 1H), 7.02-6.95 (m, 1H), 5.63 (d, J=6.9 Hz, 1H), 4.82 (q, J=7.1 Hz, 1H), 4.03 (dd, J=7.1, 4.4 Hz, 2H), 3.72 (s, 3H), 3.06 (dd, J=15.4, 6.5 Hz, 1H), 2.94-2.82 (m, 1H), 1.91 (s, 3H), 1.12 (t, J=7.1 Hz, 3H), 1.08 (s, 9H). ¹⁹F NMR (282 MHz, DMSO-d₆) δ −58.59.

### Step 4: Preparation of ethyl (3S)-3-amino-3-[1-(2-methoxy-6-methylphenyl)-3-(trifluoromethyl)pyrazol-4-yl]propanoate

A solution of ethyl (3S)-3-[1-(2-methoxy-6-methylphenyl)-3-(trifluoromethyl)pyrazol-4-yl]-3-{[(R)-2-methylpropane-2-sulfinyl]amino}propanoate (1.58 g, 3.32 mmol, 1.00 equiv.) and HCl (gas) in 1,4-dioxane (15 mL, 60.00 mmol, 4 M) in CH₂Cl₂ (6 mL) was stirred for 3 h at 25° C. under nitrogen atmosphere and then concentrated to afford ethyl (3S)-3-amino-3-[1-(2-methoxy-6-methylphenyl)-3-(trifluoromethyl)pyrazol-4-yl]propanoate (1.07 g, 86.7%) as a yellow solid. LC-MS: (ES+H, m/z): [M+H]⁺=372.2. ¹H NMR (300 MHz, DMSO-d₆) δ 7.99 (s, 1H), 7.42 (t, J=8.0 Hz, 1H), 7.08 (d, J=8.3 Hz, 1H), 6.98 (d, J=7.7 Hz, 1H), 4.36 (t, J=7.0 Hz, 1H), 4.04 (q, J=7.1 Hz, 2H), 3.74 (s, 3H), 2.72-2.62 (m, 2H), 2.19 (d, J=31.9 Hz, 2H), 1.92 (s, 3H), 1.15 (t, J=7.1 Hz, 3H). ¹⁹F NMR (282 MHz, DMSO-d₆) δ −58.67.

### Step 5: Preparation methyl (S)-3-amino-3-(1-(2-hydroxy-6-methylphenyl)-3-(trifluoromethyl)-1H-pyrazol-4-yl)propanoate

A solution of ethyl (3S)-3-amino-3-[1-(2-methoxy-6-methylphenyl)-3-(trifluoromethyl)pyrazol-4-yl]propanoate (1.00 g, 2.69 mmol, 1.00 equiv.) in CH₂Cl₂ (25 mL) was treated with BBr₃ (10 mL, 10.98 mmol, 4.08 equiv., 1M in CH₂Cl₂) for 3 h at 0° C. under nitrogen atmosphere. The reaction was quenched with ice water at 0° C. and extracted with CH₂Cl₂/MeOH (10:1, 3×100 mL). The combined organic layers were washed with brine (1×150 mL), dried over anhydrous Na₂SO₄, filtered, and concentrated. The residue was purified by silica gel column chromatography (CH₂Cl₂/MeOH (0% to 10% in 30 min)) to afford methyl (S)-3-amino-3-(1-(2-hydroxy-6-methylphenyl)-3-(trifluoromethyl)-1H-pyrazol-4-yl)propanoate (200 mg, 21.6%) as a yellow solid. LC-MS: (ES+H, m/z): [M+H]⁺=344.1.

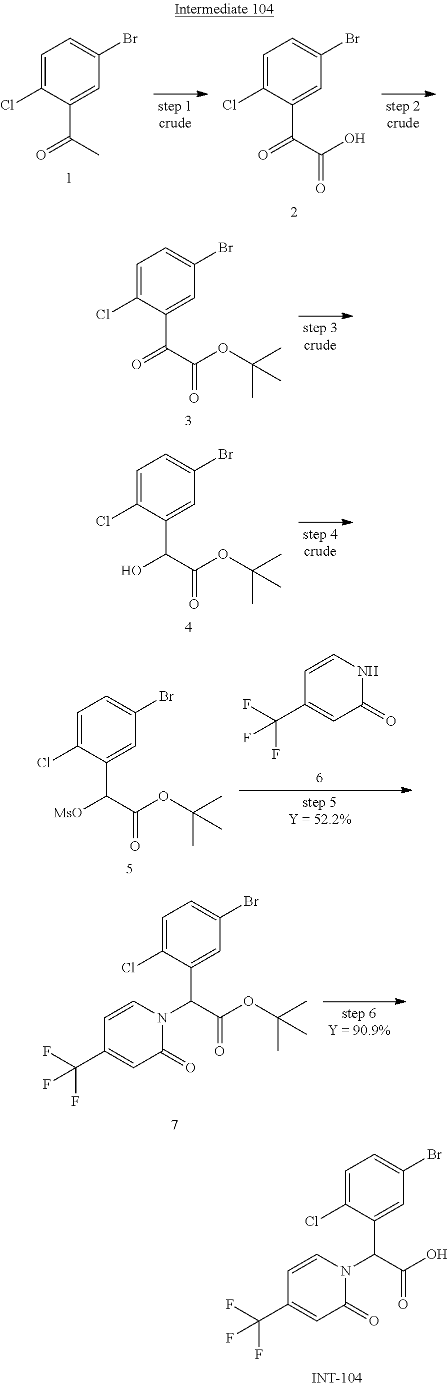

### Step 1: Preparation of (5-bromo-2-chlorophenyl)(oxo)acetic acid

To a stirred solution of 1-(5-bromo-2-chlorophenyl)ethanone (10.00 g, 21.41 mmol, 1.00 equiv.) in pyridine (150 mL) was added SeO₂ (7.13 g, 32.12 mmol, 1.50 equiv.) in portions at room temperature. The resulting mixture was stirred for 2 h at 100° C. under nitrogen atmosphere. The mixture was allowed to cool down to room temperature. The resulting mixture was filtered, and the filter cake was washed with EtOAc (200 mL). The filtrate was concentrated under reduced pressure. The residue was diluted with water (100 mL), acidified to pH 1˜2 with 6 M HCl (aq.), and extracted with CH₂Cl₂ (4×200 mL). The combined organic layers were washed with water (2×100 mL), dried over anhydrous Na₂SO₄, filtered and concentrated to afford (5-bromo-2-chlorophenyl)(oxo)acetic acid (6.4 g, crude) as a brown solid. The crude product was used in the next step directly without further purification. LC-MS: (ES−H, m/z): [M−H]⁻=260.95. ¹H NMR (400 MHz, DMSO-d₆) δ 7.93 (d, J=2.6 Hz, 1H), 7.85 (dd, J=8.6, 2.5 Hz, 1H), 7.58 (d, J=8.6 Hz, 1H).

### Step 2: Preparation of tert-butyl 2-(5-bromo-2-chlorophenyl)-2-oxoacetate

To a stirred solution of (5-bromo-2-chlorophenyl)(oxo)acetic acid (6.40 g, 24.29 mmol, 1.00 equiv., crude) in CH₂Cl₂ (200 mL) was added N,N′-diisopropyltert-butoxymethanimidamide (9.73 g, 48.58 mmol, 2.00 equiv.) in portions at room temperature. The resulting mixture was stirred overnight at room temperature under nitrogen atmosphere. The resulting mixture was diluted with water (200 mL), and extracted with CH₂Cl₂ (3×200 mL). The combined organic layers were washed with brine (2×100 mL), dried over anhydrous Na₂SO₄, filtered, and concentrated to afford tert-butyl 2-(5-bromo-2-chlorophenyl)-2-oxoacetate (5.5 g, crude) as a light yellow solid. The crude product was used in the next step directly without further purification. ¹H NMR (400 MHz, DMSO-d₆) δ 7.92 (d, J=2.5 Hz, 1H), 7.88 (dd, J=8.6, 2.4 Hz, 1H), 7.60 (d, J=8.5 Hz, 1H), 1.52 (s, 9H).

### Step 3: Preparation of tert-butyl 2-(5-bromo-2-chlorophenyl)-2-hydroxyacetate

To a stirred solution of tert-butyl 2-(5-bromo-2-chlorophenyl)-2-oxoacetate (8.20 g, 25.65 mmol, 1.00 equiv., crude) in EtOH (100 mL) and HOAc (10 mL) was added NaBH₃CN (1.77 g, 28.22 mmol, 1.10 equiv.) in portions at room temperature. The resulting mixture was stirred overnight at room temperature. The resulting mixture was diluted with water (200 mL) and extracted with CH₂Cl₂ (3×400 mL). The combined organic layers were dried over anhydrous Na₂SO₄, filtered, and concentrated to afford tert-butyl 2-(5-bromo-2-chlorophenyl)-2-hydroxyacetate (8.2 g, crude) as a light yellow solid. The crude product was used in the next step directly without further purification. ¹H NMR (400 MHz, DMSO-d₆) δ 7.66 (d, J=2.5 Hz, 1H), 7.55 (dd, J=8.5, 2.5 Hz, 1H), 7.43 (d, J=8.5 Hz, 1H), 6.44 (d, J=5.9 Hz, 1H), 5.24 (d, J=6.0 Hz, 1H), 1.36 (s, 9H).

### Step 4: Preparation of tert-butyl 2-(5-bromo-2-chlorophenyl)-2-(methanesulfonyloxy)acetate

To a stirred solution of tert-butyl 2-(5-bromo-2-chlorophenyl)-2-hydroxyacetate (8.00 g, 24.87 mmol, 1.00 equiv., crude) in CH₂Cl₂ (400 mL) were added Et₃N (7.55 g, 74.62 mmol, 3.00 equiv.) and MsCl (4.27 g, 37.31 mmol, 1.50 equiv.) dropwise at 0° C. The resulting mixture was stirred for 3 h at room temperature under nitrogen atmosphere and then diluted with water (150 mL). The resulting mixture was extracted with CH₂Cl₂ (3×300 mL) and the combined organic layers were dried over anhydrous Na₂SO₄, filtered, and concentrated to afford tert-butyl 2-(5-bromo-2-chlorophenyl)-2-(methanesulfonyloxy)acetate (10.1 g, crude) as a light yellow solid. The crude product was used in the next step directly without further purification. ¹H NMR (400 MHz, DMSO-d₆) δ 7.76 (dd, J=6.8, 2.5 Hz, 2H), 7.63-7.58 (m, 1H), 6.28 (s, 1H), 3.41 (s, 3H), 1.46 (s, 9H).

### Step 5: Preparation of tert-butyl 2-(5-bromo-2-chlorophenyl)-2-[2-oxo-4-(trifluoromethyl)pyridin-1-yl]acetate

To a stirred solution of tert-butyl 2-(5-bromo-2-chlorophenyl)-2-(methanesulfonyloxy)acetate (10.00 g, 25.02 mmol, 1.00 equiv., crude) and 4-(trifluoromethyl)-1H-pyridin-2-one (2.04 g, 12.51 mmol, 1.00 equiv.) in CH₃CN (800 mL) was added K₂CO₃ (3.46 g, 25.02 mmol, 2.00 equiv.) in portions at room temperature under nitrogen atmosphere. The resulting mixture was stirred overnight at 60° C. under nitrogen atmosphere and then cooled to room temperature. The resulting mixture was filtered, and the filter cake was washed with EtOAc (200 mL). The combined filtrates were concentrated under reduced pressure and concentrated. The residue was purified by silica gel column chromatography (PE/EtOAc (6%˜ 15% in 20 min)) to afford tert-butyl 2-(5-bromo-2-chlorophenyl)-2-[2-oxo-4-(trifluoromethyl)pyridin-1-yl]acetate (6.1 g, 52.2%) as a white solid. LC-MS: (ES−H, m/z): [M−H]⁻=463.95. 1H NMR (400 MHz, DMSO-d₆) δ 7.81-7.78 (m, 1H), 7.68-7.61 (m, 2H), 7.59 (d, J=2.4 Hz, 1H), 7.01-6.96 (m, 1H), 6.57-6.50 (m, 2H), 1.48 (s, 9H).

### Step 6: Preparation of (5-bromo-2-chlorophenyl)[2-oxo-4-(trifluoromethyl)pyridin-1-yl]acetic acid

To a stirred solution of tert-butyl 2-(5-bromo-2-chlorophenyl)-2-[2-oxo-4-(trifluoromethyl)pyridin-1-yl]acetate (6.00 g, 12.85 mmol, 1.00 equiv.) in CH₂Cl₂ (60 mL) was added CF₃COOH (60 mL) dropwise at 0° C. under nitrogen atmosphere. The resulting mixture was stirred overnight at room temperature and then diluted with CH₂Cl₂ (100 mL). The resulting mixture was concentrated under reduced pressure and the residue was purified by reversed-phase flash chromatography (C18 gel; mobile phase, CH₃CN in water, 0% to 100% gradient in 25 min; detector, UV 254 nm)) to afford (5-bromo-2-chlorophenyl)[2-oxo-4-(trifluoromethyl)pyridin-1-yl]acetic acid (4.8 g, 90.9%) as a white solid. LC-MS: (ES+H, m/z): [M+H]⁺=409.90. ¹H NMR (400 MHz, DMSO-d₆) δ 14.14 (s, 1H), 7.74 (dd, J=8.5, 2.4 Hz, 1H), 7.65-7.60 (m, 2H), 7.58 (d, J=8.5 Hz, 1H), 6.96-6.91 (m, 1H), 6.60 (s, 1H), 6.48 (dd, J=7.4, 2.1 Hz, 1H). ¹⁹F NMR (377 MHz, DMSO-d₆) δ −65.40.

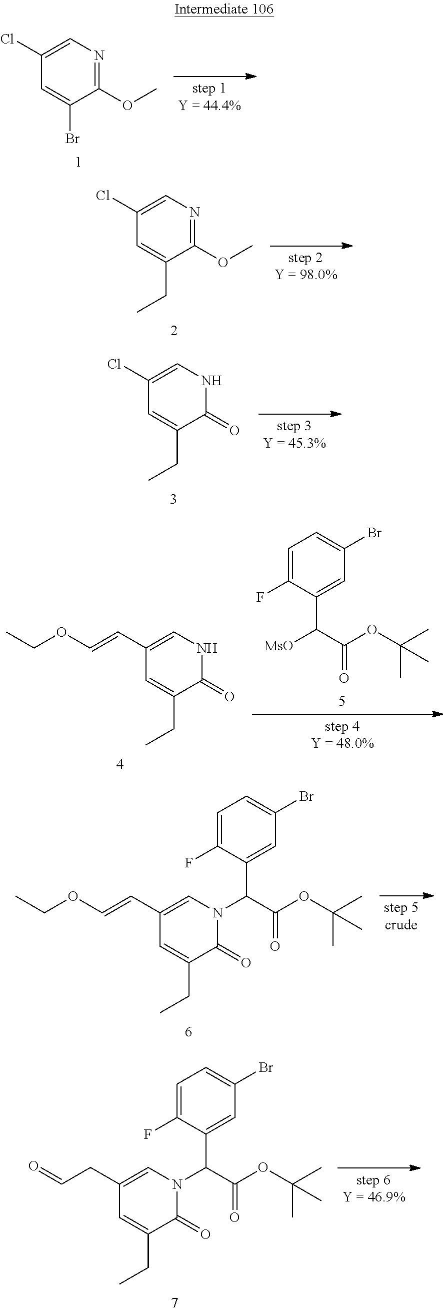

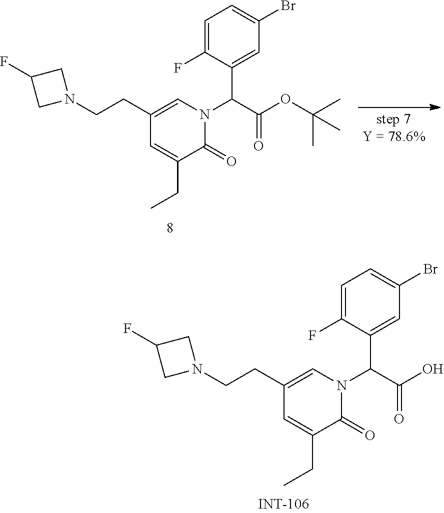 [SMILES: CCc1cc(CCN2CC(F)C2)cn(C(C(=O)OC(C)(C)C)c2cc(Br)ccc2F)c1=O ; CCc1cc(CCN2CC(F)C2)cn(C(C(=O)O)c2cc(Br)ccc2F)c1=O]

### Step 1: Preparation of 5-chloro-3-ethyl-2-methoxypyridine

To a stirred solution of 3-bromo-5-chloro-2-methoxypyridine (10.00 g, 44.55 mmol, 1.00 equiv.) in THF (100 mL) was added diethylzinc (44.55 mL, 44.55 mmol, 1.00 equiv., 1 M in hexane) dropwise at 0° C. under nitrogen atmosphere. The resulting mixture was stirred for 10 min at 0° C. under nitrogen atmosphere and then treated with Pd(PPh₃)₂Cl₂ (1.56 g, 2.23 mmol, 0.05 equiv.) in portions at 0° C. After 2 h at room temperature the reaction was quenched with water at 0° C. The resulting mixture was diluted with EtOAc (200 mL) and water (200 mL), filtered, and the filter cake was washed EtOAc (2×100 mL). The aqueous layer was extracted with EtOAc (3×300 mL) and the combined organic layers were washed with brine (3×300 mL), dried over anhydrous Na₂SO₄, filtered, and concentrated. The residue was purified by silica gel column chromatography (PE/CH₂Cl₂ (0 to 30% gradient in 40 min)) to afford 5-chloro-3-ethyl-2-methoxypyridine (3.40 g, 44.4%) as a colorless liquid. LC-MS: (ES+H, m/z): [M+H]⁺=172.1. ¹H NMR (300 MHz, CDCl₃) δ 7.94 (d, J=2.6 Hz, 1H), 7.36 (d, J=2.6, 0.8 Hz, 1H), 3.93 (s, 3H), 2.57 (q, J=7.5, 0.7 Hz, 2H), 1.19 (t, J=7.5 Hz, 3H).

### Step 2: Preparation of 5-chloro-3-ethylpyridin-2(1H)-one

A solution of 5-chloro-3-ethyl-2-methoxypyridine (2.00 g, 11.65 mmol, 1.00 equiv.) in HBr in AcOH (20 mL, 30%) was stirred overnight at 70° C. under nitrogen atmosphere. The mixture was allowed to cool down to room temperature, neutralized to pH 10 with saturated NaHCO₃ (aq.), and diluted with water (200 mL). The resulting mixture was extracted with EtOAc (3×200 mL). The combined organic layers were washed with brine (3×200 mL), dried over anhydrous Na₂SO₄, filtered, and concentrated to provide 5-chloro-3-ethylpyridin-2(1H)-one (1.80 g, 98.0%) as a white solid. LC-MS: (ES+H, m/z): [M+H]⁺=158.1. ¹H NMR (400 MHz, CDCl₃) δ 7.32 (d, J=2.7 Hz, 1H), 7.27 (d, J=1.1 Hz, 1H), 2.65-2.44 (m, 2H), 1.21 (t, J=7.5 Hz, 3H).

### Step 3: Preparation of (E)-5-(2-ethoxyvinyl)-3-ethylpyridin-2(1H)-one

To a stirred mixture of 5-chloro-3-ethylpyridin-2(1H)-one (1.80 g, 11.42 mmol, 1.00 equiv.) and 2-[(E)-2-ethoxyethenyl]-4,4,5,5-tetramethyl-1,3,2-dioxaborolane (2.49 g, 12.56 mmol, 1.10 equiv.) in dioxane (24 mL) and H₂O (1.2 mL) were added XPhos Pd G3 (0.97 g, 1.14 mmol, 0.10 equiv.), XPhos (1.09 g, 2.28 mmol, 0.20 equiv.) and TBAF (8.96 g, 34.26 mmol, 3.00 equiv.). The resulting mixture was stirred for 4 h at 100° C. under nitrogen atmosphere and then cooled to room temperature. The resulting mixture was diluted with water (200 mL) and extracted with EtOAc (3×200 mL). The combined organic layers were washed with brine (3×200 mL), dried over anhydrous Na₂SO₄, filtered, and concentrated. The residue was purified by reversed-phase flash chromatography (C18 gel; mobile phase, MeCN in water (0.1% NH₃·H₂O), 0% to 40% gradient in 30 min; detector, UV 220 nm) to provide (E)-5-(2-ethoxyvinyl)-3-ethylpyridin-2(1H)-one (1.00 g, 45.3%) as a yellow solid. LC-MS: (ES+H, m/z): [M+H]⁺=194.2. ¹H NMR (400 MHz, DMSO-d₆) δ 11.35 (s, 1H), 7.41 (d, J=2.5 Hz, 1H), 7.03 (d, J=2.6 Hz, 1H), 6.96 (d, J=13.0 Hz, 1H), 5.62 (d, J=12.9 Hz, 1H), 3.80 (q, J=7.0 Hz, 2H), 2.38 (q, J=7.4 Hz, 2H), 1.22 (t, J=7.0 Hz, 3H), 1.09 (t, J=7.4 Hz, 3H).

### Step 4: Preparation of tert-butyl (E)-2-(5-bromo-2-fluorophenyl)-2-(5-(2-ethoxyvinyl)-3-ethyl-2-oxopyridin-1(2H)-yl)acetate

To a stirred mixture of (E)-5-(2-ethoxyvinyl)-3-ethylpyridin-2(1H)-one (1.00 g, 5.18 mmol, 1.00 equiv.) and tert-butyl 2-(5-bromo-2-fluorophenyl)-2-((methylsulfonyl)oxy)acetate (2.97 g, 7.76 mmol, 1.50 equiv.) in MeCN (30 mL) was added K₂CO₃ (2.15 g, 15.53 mmol, 3.00 equiv.). The resulting mixture was stirred for 4 h at 60° C. under nitrogen atmosphere. The mixture was cooled to room temperature and filtered. After washing the filter cake with EtOAc (3×100 mL) the combined filtrates were concentrated under reduced pressure. The residue was purified by reversed-phase flash chromatography (C18 gel; mobile phase, MeCN in water (0.1% NH₃·H₂O), 40% to 70% gradient in 30 min; detector, UV 220 nm) to provide tert-butyl (E)-2-(5-bromo-2-fluorophenyl)-2-(5-(2-ethoxyvinyl)-3-ethyl-2-oxopyridin-1(2H)-yl)acetate (1.20 g, 48.2%) as a yellow solid. LC-MS: (ES+H, m/z): [M+H]⁺=480. ¹H NMR (300 MHz, DMSO-d₆) δ 7.70-7.63 (m, 1H), 7.52 (dd, J=6.5, 2.5 Hz, 2H), 7.30 (dd, J=10.0, 8.8 Hz, 1H), 7.16 (d, J=2.4 Hz, 1H), 7.01 (d, J=13.0 Hz, 1H), 6.40 (s, 1H), 5.60 (d, J=13.0 Hz, 1H), 3.81 (q, J=7.0 Hz, 2H), 2.48-2.36 (m, 2H), 1.39 (s, 9H), 1.21 (t, J=7.0 Hz, 3H), 1.12 (t, J=7.4 Hz, 3H).

### Step 5: Preparation of tert-butyl 2-(5-bromo-2-fluorophenyl)-2-(3-ethyl-2-oxo-5-(2-oxoethyl)pyridin-1(2H)-yl)acetate

To a stirred solution of tert-butyl (E)-2-(5-bromo-2-fluorophenyl)-2-(5-(2-ethoxyvinyl)-3-ethyl-2-oxopyridin-1(2H)-yl)acetate (1.00 g, 2.08 mmol, 1.00 equiv.) in THF (20 mL) was added HCl (gas) in 1,4-dioxane (20 mL, 80.00 mmol, 38.43 equiv., 4 M) dropwise at room temperature under nitrogen atmosphere. After 2 h the reaction was concentrated to provide tert-butyl 2-(5-bromo-2-fluorophenyl)-2-(3-ethyl-2-oxo-5-(2-oxoethyl)pyridin-1(2H)-yl)acetate (1.20 g, crude) as a yellow oil. LC-MS: (ES+H, m/z): [M+H]⁺=452.1.

### Step 6: Preparation of tert-butyl 2-(5-bromo-2-fluorophenyl)-2-(3-ethyl-5-(2-(3-fluoroazetidin-1-yl)ethyl)-2-oxopyridin-1(2H)-yl)acetate

To a stirred mixture of tert-butyl 2-(5-bromo-2-fluorophenyl)-2-(3-ethyl-2-oxo-5-(2-oxoethyl)pyridin-1(2H)-yl)acetate (1.20 g, assumed 100% yield, 2.65 mmol, 1.00 equiv.) and 3-fluoroazetidine hydrochloride (592 mg, 5.31 mmol, 2.00 equiv.) in EtOH (20 mL) was added AcOH (16 mg, 0.27 mmol, 0.10 equiv.). The resulting mixture was stirred for 1 h at room temperature under nitrogen atmosphere and then NaBH₃CN (333 mg, 5.31 mmol, 2.00 equiv.) was added in portions at room temperature. After 1 h, the reaction was quenched with water at 0° C., further diluted with water (200 mL) and extracted with EtOAc (3×200 mL). The combined organic layers were washed with brine (3×200 mL), dried over anhydrous Na₂SO₄, filtered, and concentrated. The residue was purified by silica gel column chromatography (PE/EtOAc (50% to 80% gradient in 30 min) to afford tert-butyl 2-(5-bromo-2-fluorophenyl)-2-(3-ethyl-5-(2-(3-fluoroazetidin-1-yl)ethyl)-2-oxopyridin-1(2H)-yl)acetate (500 mg, 46.9%) as a yellow solid. LC-MS: (ES+H, m/z): [M+H]⁺=511.2. ¹H NMR (400 MHz, CD₃OD) δ 7.67-7.55 (m, 2H), 7.32 (dt, J=2.2, 1.0 Hz, 1H), 7.18 (dd, J=9.9, 8.7 Hz, 1H), 7.10 (d, J=2.4 Hz, 1H), 6.55 (s, 1H), 5.19-4.95 (m, 1H), 3.61-3.47 (m, 2H), 3.23-3.09 (m, 2H), 2.67 (t, J=7.2 Hz, 2H), 2.58-2.51 (m, 2H), 2.42 (t, J=7.2 Hz, 2H), 1.47 (s, 9H), 1.19 (t, J=7.5 Hz, 3H).

### Step 7: Preparation of 2-(5-bromo-2-fluorophenyl)-2-(3-ethyl-5-(2-(3-fluoroazetidin-1-yl)ethyl)-2-oxopyridin-1(2H)-yl)acetic acid

To a stirred solution of tert-butyl 2-(5-bromo-2-fluorophenyl)-2-(3-ethyl-5-(2-(3-fluoroazetidin-1-yl)ethyl)-2-oxopyridin-1(2H)-yl)acetate (500 mg, 0.98 mmol, 1.00 equiv.) in CH₂Cl₂ (2 mL) was added TFA (2 mL, 26.93 mmol, 27.54 equiv.) dropwise at room temperature under nitrogen atmosphere. The resulting mixture was stirred for 2 h at room temperature and then concentrated. The residue was purified by reversed-phase flash chromatography (C18 gel; mobile phase, MeCN in water, 0 to 40% gradient in 30 min; detector, UV 220 nm) to provide 2-(5-bromo-2-fluorophenyl)-2-(3-ethyl-5-(2-(3-fluoroazetidin-1-yl)ethyl)-2-oxopyridin-1(2H)-yl)acetic acid (350 mg, 78.6%) as a yellow solid. LC-MS: (ES+H, m/z): [M+H]⁺=455.0. ¹H NMR (300 MHz, CD₃OD) δ 7.68-7.60 (m, 1H), 7.53 (ddd, J=8.7, 4.5, 2.5 Hz, 1H), 7.31 (dt, J=2.3, 1.0 Hz, 1H), 7.19 (d, J=2.5 Hz, 1H), 7.07 (dd, J=9.8, 8.7 Hz, 1H), 6.84 (s, 1H), 5.40-5.18 (m, 1H), 4.57-4.04 (m, 4H), 3.55-3.37 (m, 2H), 2.78-2.50 (m, 4H), 1.20 (t, J=7.5 Hz, 3H).

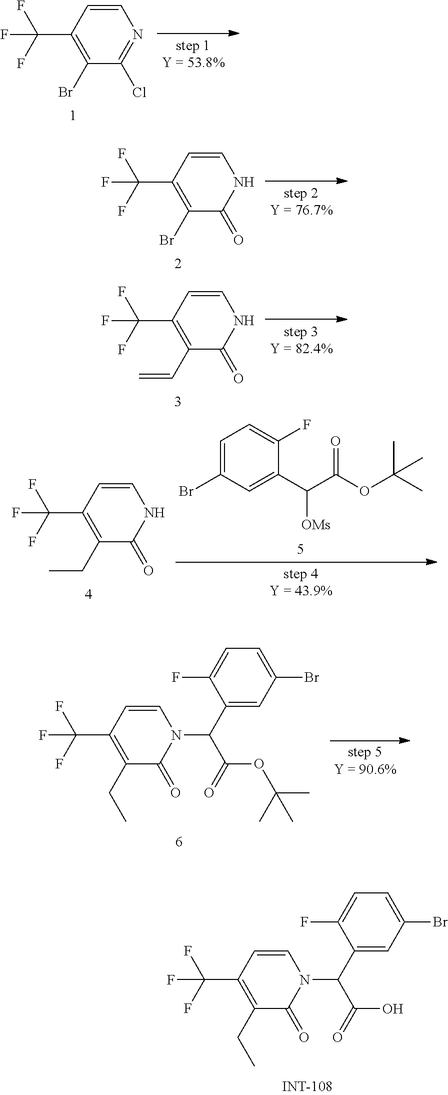

### Step 1: Preparation of 3-bromo-4-(trifluoromethyl)-1H-pyridin-2-one

To a stirred solution of 3-bromo-2-chloro-4-(trifluoromethyl)pyridine (2.00 g, 7.67 mmol, 1.00 equiv.) in 2-methyl-2-butanol (10 mL) was added t-BuOK (4.31 g, 38.39 mmol, 5.00 equiv.) at room temperature under nitrogen atmosphere. The resulting mixture was stirred for 2 h at 80° C. and then cooled to room temperature and concentrated. The residue was purified by silica gel column chromatography (CH₂Cl₂/MeOH (0˜10%, 20 min) to afford 3-bromo-4-(trifluoromethyl)-1H-pyridin-2-one (1 g, 53.8%) as off-white solid. LC-MS: (ES+H, m/z): [M+H]⁺=242.0. ¹H NMR (400 MHz, DMSO-d₆) δ 12.72 (s, 1H), 7.76-7.67 (m, 1H), 6.49 (d, J=6.7 Hz, 1H). ¹⁹F NMR (377 MHz, DMSO-d₆) δ −63.50.

### Step 2: Preparation of 3-ethenyl-4-(trifluoromethyl)-1H-pyridin-2-one

To a stirred mixture of 3-bromo-4-(trifluoromethyl)-1H-pyridin-2-one (1.00 g, 4.13 mmol, 1.00 equiv.) and 2-ethenyl-4,4,5,5-tetramethyl-1,3,2-dioxaborolane (1.91 g, 12.39 mmol, 3.00 equiv.) in 1,4-dioxane (10 mL) and H₂O (1 mL) were added Pd(dppf)Cl₂ CH₂Cl₂ (168 mg, 0.20 mmol, 0.05 equiv.) and K₂CO₃ (1.14 g, 8.26 mmol, 2.00 equiv.) at room temperature under nitrogen atmosphere. The resulting mixture was stirred for 2 h at 100° C. and then cooled to room temperature. The resulting mixture was diluted with ethyl acetate (20 mL) and washed with water (3×20 mL), brine (2×20 mL), dried over anhydrous Na₂SO₄, filtered, and concentrated. The residue was purified by silica gel column chromatography (PE/EtOAc (30˜50%, 20 min) to afford 3-ethenyl-4-(trifluoromethyl)-1H-pyridin-2-one (600 mg, 76.7%) as a yellow oil. LC-MS: (ES+H, m/z): [M+H]⁺=190.0. ¹H NMR (400 MHz, DMSO-d₆) δ7.60-7.54 (m, 1H), 6.77-6.60 (m, 2H), 6.44 (d, J=6.8 Hz, 1H), 5.60 (dd, J=11.3, 3.5 Hz, 1H). ¹⁹F NMR (377 MHz, DMSO-d₆) δ −59.48.

### Step 3: Preparation of 3-ethyl-4-(trifluoromethyl)-1H-pyridin-2-one

To a stirred mixture of 3-ethenyl-4-(trifluoromethyl)-1H-pyridin-2-one (600 mg, 3.17 mmol, 1.00 equiv.) in MeOH (6 mL) was added Pd/C (607 mg, 5.71 mmol, 1.80 equiv., 10%) at room temperature under nitrogen atmosphere. The resulting mixture was stirred for 1 h at room temperature under hydrogen atmosphere. The resulting mixture was filtered and the filter cake was washed with MeOH (3×10 mL). The filtrate was concentrated under reduced pressure to afford 3-ethyl-4-(trifluoromethyl)-1H-pyridin-2-one (500 mg, 82.4%) as an off-white solid. LC-MS: (ES+H, m/z): [M+H]⁺=192.0. ¹H NMR (400 MHz, DMSO-d₆) δ 7.48 (dd, J=6.9, 1.0 Hz, 1H), 6.32 (d, J=6.9 Hz, 1H), 2.59-2.52 (m, 2H), 1.06 (t, J=7.3 Hz, 3H). ¹⁹F NMR (377 MHz, DMSO-d₆) δ −60.58.

### Step 4: Preparation of tert-butyl 2-(5-bromo-2-fluorophenyl)-2-[3-ethyl-2-oxo-4-(trifluoromethyl)pyridin-1-yl]acetate

To a stirred mixture of 3-ethyl-4-(trifluoromethyl)-1H-pyridin-2-one (500 mg, 2.61 mmol, 1.00 equiv.) and tert-butyl 2-(5-bromo-2-fluorophenyl)-2-(methanesulfonyloxy)acetate (1.00 g, 2.61 mmol, 1.00 equiv.) in MeCN (15 mL) was added K₂CO₃ (723 mg, 5.23 mmol, 2.00 equiv.) at room temperature under nitrogen atmosphere. The resulting mixture was stirred for 2 h at 60° C. and then cooled to room temperature, diluted with water (20 mL), and extracted with EtOAc (2×20 mL). The combined organic layers were washed with brine (2×20 mL), dried over anhydrous Na₂SO₄, filtered, and concentrated. The residue was purified by reversed-phase flash chromatography (C18 gel; mobile phase, MeCN in water (10 mmol/L NH₄HCO₃), 50% to 70% gradient in 20 min; detector, UV 254/220 nm) to afford tert-butyl 2-(5-bromo-2-fluorophenyl)-2-[3-ethyl-2-oxo-4-(trifluoromethyl)pyridin-1-yl]acetate (550 mg, 43.9%) as colorless oil. LC-MS: (ES+H, m/z): [M+H]⁺=478.1. ¹H NMR (400 MHz, DMSO-d₆) δ 7.74-7.60 (m, 3H), 7.34 (t, J=10.0, 8.8 Hz, 1H), 6.54 (s, 1H), 6.48 (d, J=7.5 Hz, 1H), 2.69-2.55 (m, 2H), 1.41 (s, 9H), 1.08 (t, J=7.3 Hz, 3H). ¹⁹F NMR (377 MHz, DMSO-d₆) δ −60.83, −116.49.

### Step 5: Preparation of 2-(5-bromo-2-fluorophenyl)-2-(3-ethyl-2-oxo-4-(trifluoromethyl)pyridin-1(2H)-yl)acetic acid

To a stirred mixture of tert-butyl 2-(5-bromo-2-fluorophenyl)-2-[3-ethyl-2-oxo-4-(trifluoromethyl)pyridin-1-yl]acetate (550 mg, 1.15 mmol, 1.00 equiv.) in CH₂Cl₂ (5 mL) was added CF₃COOH (5 mL) at room temperature under nitrogen atmosphere. The resulting mixture was stirred for 1 h at room temperature and then concentrated. The residue was purified by reversed-phase flash chromatography (C18 gel; mobile phase, MeCN in water, 10% to 40% gradient in 20 min; detector, UV 254/220 nm) to afford 2-(5-bromo-2-fluorophenyl)[3-ethyl-2-oxo-4-(trifluoromethyl)pyridin-1-yl]acetic acid (400 mg, 90.6%) as an off-white solid. LC-MS: (ES+H, m/z): [M+H]⁺=422.0.

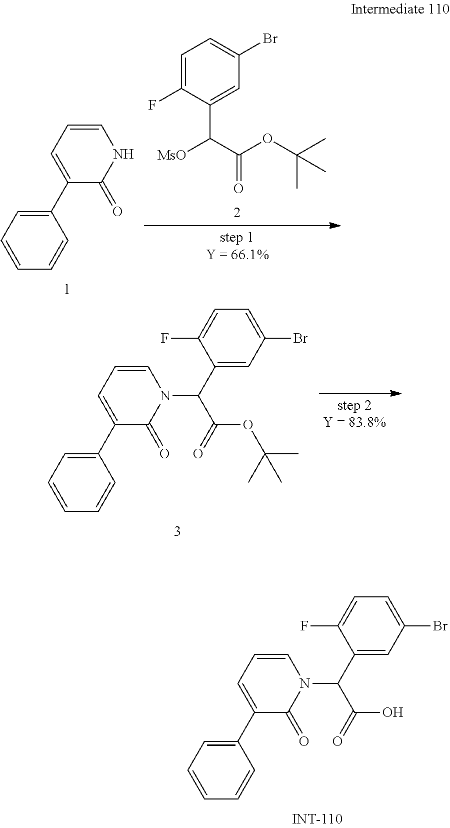 [SMILES: O=c1[nH]cccc1-c1ccccc1 ; CC(C)(C)OC(=O)C(OS(C)(=O)=O)c1cc(Br)ccc1F ; CC(C)(C)OC(=O)C(c1cc(Br)ccc1F)n1cccc(-c2ccccc2)c1=O ; O=C(O)C(c1cc(Br)ccc1F)n1cccc(-c2ccccc2)c1=O]

### Step 1: Preparation of tert-butyl 2-(5-bromo-2-fluorophenyl)-2-(2-oxo-3-phenylpyridin-1(2H)-yl)acetate

To a stirred solution of 3-phenyl-1H-pyridin-2-one (500 mg, 2.92 mmol, 1.00 equiv.) and tert-butyl 2-(5-bromo-2-fluorophenyl)-2-(methanesulfonyloxy)acetate (1.34 g, 3.51 mmol, 1.20 equiv.) in MeCN (10 mL) was added K₂CO₃ (1.21 g, 8.76 mmol, 3.00 equiv.) at 25° C. The resulting mixture was stirred for 16 h at 60° C. under nitrogen atmosphere and then cooled to room temperature and filtered. The filter cake was washed with EtOAc (3×10 mL). and the combined filtrates were concentrated under reduced pressure. The residue was purified by silica gel column chromatography (PE/EtOAc (0 to 10% gradient in 30 min) to afford the crude product which was further purified by reversed-phase flash chromatography (C18 gel; mobile phase, MeCN in water (0.1% HCOOH), 70% to 100% gradient in 20 min; detector, UV 254 nm) to afford tert-butyl 2-(5-bromo-2-fluorophenyl)-2-(2-oxo-3-phenylpyridin-1-yl)acetate (900 mg, 66.16%) as a white solid. LC-MS: (ES+H, m/z): [M+H]⁺=458.00. ¹H NMR (400 MHz, DMSO-d₆) δ 7.80-7.61 (m, 5H), 7.54 (dd, J=6.9, 1.9 Hz, 1H), 7.46-7.37 (m, 2H), 7.37-7.27 (m, 2H), 6.65-6.55 (m, 1H), 6.41 (t, J=6.9 Hz, 1H), 1.42 (s, 9H). ¹⁹F NMR (377 MHz, DMSO-d₆) δ −116.61.

### Step 2: Preparation of 2-(5-bromo-2-fluorophenyl)-2-(2-oxo-3-phenylpyridin-1(2H)-yl)acetic acid

A solution of tert-butyl 2-(5-bromo-2-fluorophenyl)-2-(2-oxo-3-phenylpyridin-1-yl)acetate (800 mg, 1.75 mmol, 1.00 equiv.) in HCl (gas) in 1,4-dioxane (15 mL, 4 M) was stirred for 16 h at 50° C. under nitrogen atmosphere and then cooled to room temperature and concentrated. The residue was purified by reversed-phase flash chromatography (C18 gel; mobile phase, MeCN in water, 40% to 60% gradient in 20 min; detector, UV 254 nm) to afford 2-(5-bromo-2-fluorophenyl)(2-oxo-3-phenylpyridin-1-yl)acetic acid (600 mg, 83.8%) as a white solid. LC-MS: (ES−H, m/z): [M+H]⁺=401.95. ¹H NMR (400 MHz, DMSO-d₆) δ 13.70 (s, 1H), 7.75-7.63 (m, 5H), 7.57 (dd, J=6.9, 1.9 Hz, 1H), 7.43-7.37 (m, 2H), 7.36-7.29 (m, 2H), 6.66 (s, 1H), 6.40 (t, J=6.9 Hz, 1H).

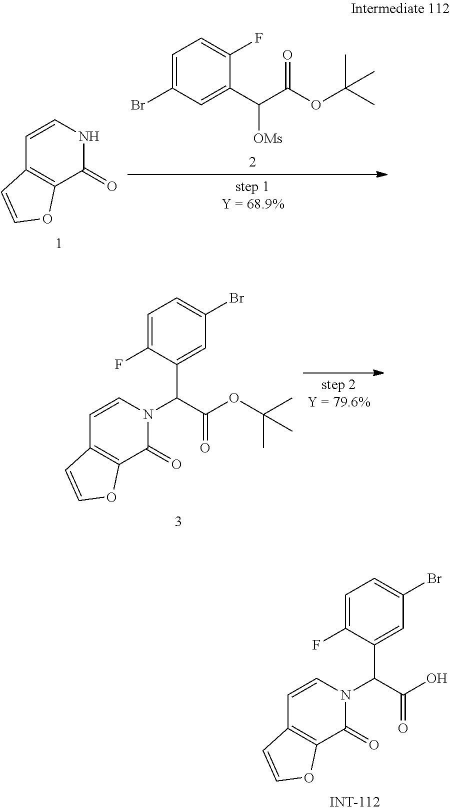 [SMILES: O=c1[nH]ccc2ccoc12 ; CC(C)(C)OC(=O)C(OS(C)(=O)=O)c1cc(Br)ccc1F ; CC(C)(C)OC(=O)C(c1cc(Br)ccc1F)n1ccc2ccoc2c1=O ; O=C(O)C(c1cc(Br)ccc1F)n1ccc2ccoc2c1=O]

### Step 1: Preparation of tert-butyl 2-(5-bromo-2-fluorophenyl)-2-(7-oxofuro[2,3-c]pyridin-6(7H)-yl)acetate

To a stirred mixture of furo[2,3-c]pyridin-7(6H)-one (500 mg, 3.70 mmol, 1.00 equiv.) and K₂CO₃ (1.53 g, 11.100 mmol, 3.00 equiv.) in MeCN (30 mL) was added tert-butyl 2-(5-bromo-2-fluorophenyl)-2-(methanesulfonyloxy)acetate (1.99 g, 5.180 mmol, 1.40 equiv.) at 25° C. The resulting mixture was stirred for 2 h at 80° C. under nitrogen atmosphere. The mixture was allowed to cool down to 25° C. and diluted with acetonitrile (30 mL). The resulting mixture was filtered, and the filter cake was washed with ethyl acetate (3×5 mL). The combined filtrates were concentrated under reduced pressure and the residue was purified by silica gel chromatography (EtOAc in PE, 0% to 15% gradient in 30 min, hold at 9%; detector, UV 254/280 nm)) to afford tert-butyl 2-(5-bromo-2-fluorophenyl)-2-(7-oxofuro[2,3-c]pyridin-6(7H)-yl)acetate (1.10 g, 68.9%) as a yellow solid. LC-MS: (ES+H, m/z): [M+H]⁺=421.9. ¹H NMR (400 MHz, DMSO-d₆) δ 8.20 (d, J=2.0 Hz, 1H), 7.74-7.65 (m, 1H), 7.60 (dd, J=6.5, 2.5 Hz, 1H), 7.40-7.29 (m, 2H), 6.94 (d, J=2.0 Hz, 1H), 6.69 (s, 1H), 6.66 (d, J=7.2 Hz, 1H), 1.42 (s, 9H). ¹⁹F NMR (377 MHz, DMSO-d₆) δ −116.65.

### Step 2: Preparation of 2-(5-bromo-2-fluorophenyl)-2-(7-oxofuro[2,3-c]pyridin-6(7H)-yl)acetic acid

A solution of tert-butyl 2-(5-bromo-2-fluorophenyl)-2-(3-oxoisoquinolin-2-yl)acetate (1.00 g, 2.31 mmol, 1.00 equiv.) in CH₂Cl₂ (10 mL) and TFA (10 mL) was stirred for 2.5 h at 25° C. under nitrogen atmosphere and then diluted with CH₂Cl₂ (200 mL). The resulting mixture was washed with water (1×100 mL) and then concentrated under reduced pressure. The crude product was purified by reverse phase flash chromatography (C18 gel; mobile phase, MeCN in water, 20% to 80% gradient in 30 min; detector, UV 254 nm)) to afford 2-(5-bromo-2-fluorophenyl)-2-(7-oxofuro[2,3-c]pyridin-6(7H)-yl)acetic acid (700 mg, 79.6%) as a white solid. LC-MS: (ES+H, m/z): [M+H]⁺=365.95. ¹H NMR (300 MHz, DMSO-d₆) δ 13.78 (s, 1H), 8.18 (d, J=2.0 Hz, 1H), 7.73-7.61 (m, 2H), 7.41-7.23 (m, 2H), 6.92 (d, J=2.0 Hz, 1H), 6.71 (s, 1H), 6.64 (d, J=7.2 Hz, 1H). ¹⁹F NMR (282 MHz, DMSO-d₆) δ −116.70.

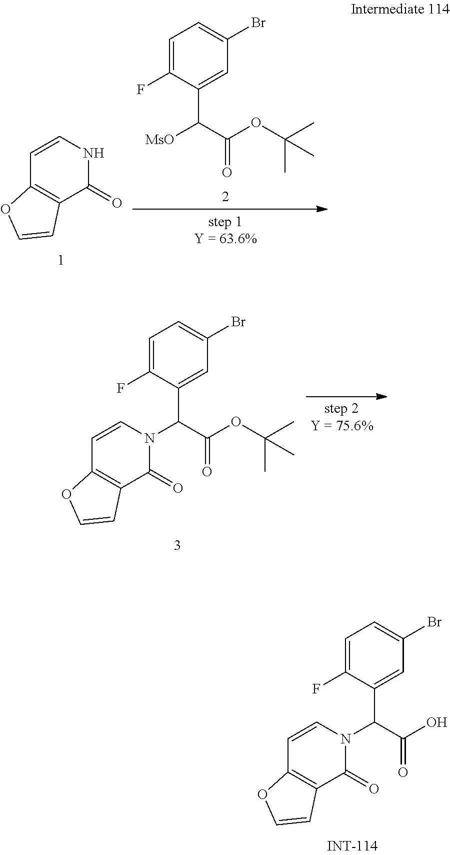 [SMILES: O=c1[nH]ccc2occc12 ; CC(C)(C)OC(=O)C(OS(C)(=O)=O)c1cc(Br)ccc1F ; CC(C)(C)OC(=O)C(c1cc(Br)ccc1F)n1ccc2occc2c1=O ; O=C(O)C(c1cc(Br)ccc1F)n1ccc2occc2c1=O]

### Step 1: Preparation of tert-butyl 2-(5-bromo-2-fluorophenyl)-2-(4-oxofuro[3,2-c]pyridin-5(4H)-yl)acetate

To a stirred mixture of 5H-furo[3,2-c]pyridin-4-one (500 mg, 3.70 mmol, 1.00 equiv.) and tert-butyl 2-(5-bromo-2-fluorophenyl)-2-(methanesulfonyloxy)acetate (2.41 g, 6.29 mmol, 1.70 equiv.) in MeCN (30 mL) was added K₂CO₃ (1.53 g, 11.10 mmol, 3.00 equiv.). The resulting mixture was stirred for 5 h at 80° C. under nitrogen atmosphere and then cooled to room temperature. The reaction mixture was filtered and the filter cake was washed with EtOAc (3×3 mL). The filtrate was concentrated under reduced pressure and the residue was purified by silica gel column chromatography (PE/EtOAc (0 to 20% gradient in 20 min) to afford the crude product. which was further purified by reversed-phase flash chromatography (C18 gel; mobile phase, MeCN in water (0.1% HCOOH), 60% to 100% gradient in 15 min; detector, UV 254 nm) to afford tert-butyl 2-(5-bromo-2-fluorophenyl)-2-(4-oxofuro[3,2-c]pyridin-5(4H)-yl)acetate (1.00 g, 63.6%) as a colourless oil. LC-MS: (ES+H, m/z): [M+H]⁺=422.0. ¹H NMR (300 MHz, DMSO-d₆) δ 7.95 (d, J=2.2 Hz, 1H), 7.74-7.64 (m, 1H), 7.66-7.55 (m, 1H), 7.46 (d, J=7.6 Hz, 1H), 7.41-7.26 (m, 1H), 7.10-6.97 (m, 1H), 6.89-6.74 (m, 1H), 6.66 (s, 1H), 1.42 (s, 9H). ¹⁹F NMR (282 MHz, DMSO-d₆) δ −116.73.

### Step 2: Preparation of 2-(5-bromo-2-fluorophenyl)-2-(4-oxofuro[3,2-c]pyridin-5(4H)-yl)acetic acid

A solution of tert-butyl 2-(5-bromo-2-fluorophenyl)-2-(4-oxofuro[3,2-c]pyridin-5(4H)-yl)acetate (1.00 g, 2.37 mmol, 1.00 equiv.) in CH₂Cl₂ (10 mL) and TFA (10 mL) was stirred for 2 h at 25° C. under nitrogen atmosphere and then diluted with CH₂Cl₂ (30 mL). The resulting mixture was washed with water (3×50 mL) and concentrated. The solids were washed with water (3×2 mL) to provide 2-(5-bromo-2-fluorophenyl)-2-(4-oxofuro[3,2-c]pyridin-5(4H)-yl)acetic acid (680 mg, 75.6%) as a white solid. LC-MS: (ES+H, m/z): [M+H]⁺=365.90. ¹H NMR (300 MHz, DMSO-d₆) δ 7.94 (d, J=2.1 Hz, 1H), 7.76-7.60 (m, 2H), 7.51 (d, J=7.6 Hz, 1H), 7.38-7.25 (m, 1H), 7.08-6.97 (m, 1H), 6.88-6.77 (m, 1H), 6.72 (s, 1H). ¹⁹F NMR (282 MHz, DMSO-d₆) δ −116.75.

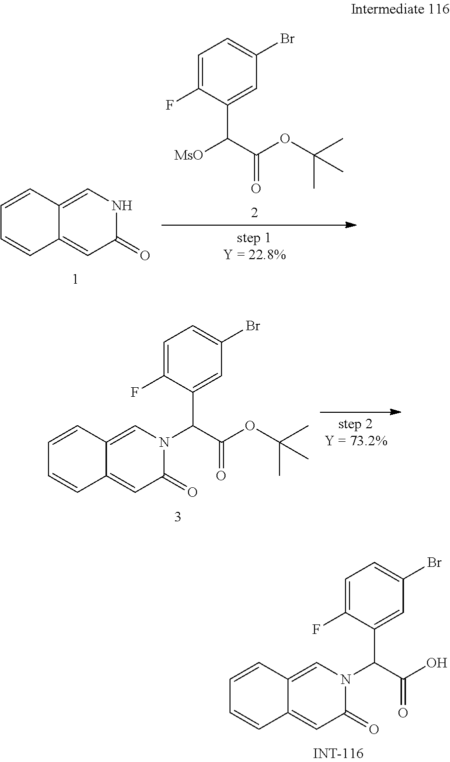 [SMILES: CC(C)(C)OC(=O)C(OS(C)(=O)=O)c1cc(Br)ccc1F ; O=c1cc2ccccc2c[nH]1 ; CC(C)(C)OC(=O)C(c1cc(Br)ccc1F)n1cc2ccccc2cc1=O ; O=C(O)C(c1cc(Br)ccc1F)n1cc2ccccc2cc1=O]

### Step 1: Preparation of tert-butyl 2-(5-bromo-2-fluorophenyl)-2-(3-oxoisoquinolin-2(3H)-yl)acetate

To a stirred solution of 2H-isoquinolin-3-one (1.50 g, 10.33 mmol, 1.00 equiv.) and tert-butyl 2-(5-bromo-2-fluorophenyl)-2-(methanesulfonyloxy)acetate (4.75 g, 12.40 mmol, 1.20 equiv.) in MeCN (150 mL) was added K₂CO₃ (7.14 g, 51.67 mmol, 5.00 equiv.) at 25° C. under nitrogen atmosphere. The resulting mixture was stirred for 2 h at 80° C. and then cooled to room temperature and filtered. The filter cake was washed with EtOAc (3×20 mL) and the filtrate was concentrated under reduced pressure. The residue was purified by silica gel column chromatography (PE/EtOAc (0 to 30% gradient in 30 min) to afford tert-butyl 2-(5-bromo-2-fluorophenyl)-2-(3-oxoisoquinolin-2-yl)acetate (1.2 g, 22.8%) as a yellow solid. LC-MS: (ES+H, m/z): [M+H]⁺=432.0. ¹H NMR (300 MHz, DMSO-d₆) δ 8.58 (s, 1H), 7.76-7.57 (m, 3H), 7.40-7.24 (m, 3H), 6.96-6.89 (m, 1H), 6.79 (s, 1H), 6.67 (d, J=1.1 Hz, 1H), 1.41 (s, 9H). ¹⁹F NMR (282 MHz, DMSO-d₆) δ −117.20.

### Step 2: Preparation of 2-(5-bromo-2-fluorophenyl)-2-(3-oxoisoquinolin-2(3H)-yl)acetic acid

To a stirred mixture of tert-butyl 2-(5-bromo-2-fluorophenyl)-2-(3-oxoisoquinolin-2-yl)acetate (1.20 g, 2.78 mmol, 1.00 equiv.) in CH₂Cl₂ (8 mL) was added CF₃HCOOH (8 mL) dropwise at 25° C. under nitrogen atmosphere. After 3 h, the reaction was diluted with CH₂Cl₂ (30 mL) and washed with water (3×20 mL). After filtration, the filtrate was concentrated under reduced pressure. The residue product was purified by reverse phase flash chromatography (C18 gel; mobile phase, MeCN in water, 0% to 60% gradient in 20 min; detector, UV 220 nm) to afford 2-(5-bromo-2-fluorophenyl)(3-oxoisoquinolin-2-yl)acetic acid (900 mg, 73.2%) as a yellow solid. LC-MS: (ES+H, m/z): [M+H]⁺=376.0.

### Synthesis of Examples

### Example 1

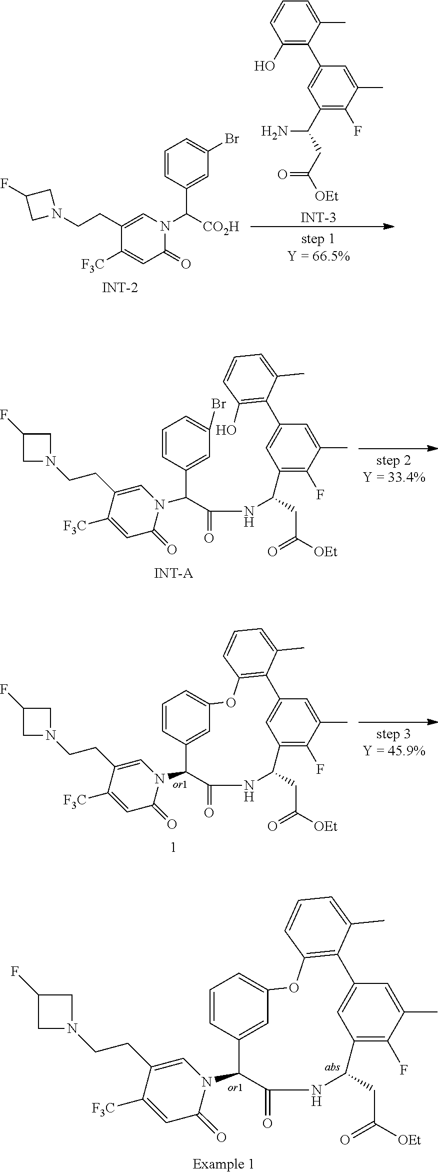

### Step 1: Preparation of ethyl (3S)-3-(2-(3-bromophenyl)-2-(5-(2-(3-fluoroazetidin-1-yl)ethyl)-2-oxo-4-(trifluoromethyl)pyridin-1(2H)-yl)acetamido)-3-(4-fluoro-2′-hydroxy-5,6′-dimethyl-[1,1′-biphenyl]-3-yl)propanoate

To a stirred mixture of (3-bromophenyl)({5-[2-(3-fluoroazetidin-1-yl)ethyl]-2-oxo-4-(trifluoromethyl)pyridin-1-yl})acetic acid (700 mg, 1.46 mmol, 1.00 equiv) in CH₃CN (15 mL) were added HOBT (257 mg, 1.90 mmol, 1.30 equiv) and EDCI (421 mg, 2.20 mmol, 1.50 equiv) in portions at room temperature under nitrogen atmosphere. The resulting mixture was stirred for 1 h at room temperature under nitrogen atmosphere. To the above mixture was added ethyl (3S)-3-amino-3-{4-fluoro-2′-hydroxy-5,6′-dimethyl-[1,1′-biphenyl]-3-yl}propanoate (600 mg, crude, in 0.2 mL Et₃N) in CH₃CN (6 mL) dropwise over 5 min at 0° C. The resulting mixture was stirred overnight at room temperature. The reaction was monitored by LCMS. The resulting mixture was concentrated under vacuum. The residue was purified by silica gel column chromatography, eluted with CH₂Cl₂/MeOH (0% to 5% gradient in 20 min) to afford ethyl (3S)-3-(2-(3-bromophenyl)-2-(5-(2-(3-fluoroazetidin-1-yl)ethyl)-2-oxo-4-(trifluoromethyl)pyridin-1(2H)-yl)acetamido)-3-(4-fluoro-2′-hydroxy-5,6′-dimethyl-[1,1′-biphenyl]-3-yl)propanoate (700 mg, 65.5%) as a yellow solid. LC-MS: (ES+H, m/z): [M+H]⁺=790.3. ¹H NMR (300 MHz, CD₃OD) δ 7.56-7.19 (m, 4H), 7.12-7.04 (m, 1H), 6.96-6.80 (m, 2H), 6.84-6.73 (m, 1H), 6.69-6.55 (m, 3H), 5.68-5.48 (m, 1H), 4.96 (q, J=5.2 Hz, 1H), 3.98-3.92 (m, 3H), 3.39-3.33 (m, 2H), 3.02-2.92 (m, 2H), 2.78 (dd, J=17.1, 7.4 Hz, 2H), 2.51-2.29 (m, 4H), 2.18 (t, J=2.7 Hz, 3H), 1.84 (d, J=11.3 Hz, 3H), 1.07-1.01 (m, 3H).

### Step 2: Preparation of ethyl 2-((5R*, 8S)-1⁴-fluoro-5-(5-(2-(3-fluoroazetidin-1-yl)ethyl)-2-oxo-4-(trifluoromethyl)pyridin-1(2H)-yl)-1⁵,2⁶-dimethyl-6-oxo-3-oxa-7-aza-1,4(1,3),2(1,2)-tribenzenacyclooctaphane-8-yl)acetate

To a stirred solution of ethyl (3S)-3-[2-(3-bromophenyl)-2-{5-[2-(3-fluoroazetidin-1-yl)ethyl]-2-oxo-4-(trifluoromethyl)pyridin-1-yl}acetamido]-3-{4-fluoro-2′-hydroxy-5,6′-dimethyl-[1,1′-biphenyl]-3-yl}propanoate (500 mg, 0.63 mmol, 1 equiv) in dioxane (15 mL) were added CuI (60 mg, 0.32 mmol, 0.50 equiv) and K₂CO₃ (175 mg, 1.26 mmol, 2.00 equiv) in portions at room temperature under nitrogen atmosphere. The resulting mixture was stirred overnight at 100° C. under nitrogen atmosphere and then cooled to room temperature. The resulting mixture was concentrated under reduced pressure. The residue was purified by silica gel column chromatography, eluted with CH₂Cl₂/MeOH (0% to 5% gradient in 20 min) to afford (300 mg, crude). The residue was further purified by reversed-phase flash chromatography (C18 gel; mobile phase, MeCN in water (0.1% HCO₂H), 0% to 50% gradient in 40 min; detector, UV 254 nm) to provide ethyl 2-((5R*,8S)-1⁴-fluoro-5-(5-(2-(3-fluoroazetidin-1-yl)ethyl)-2-oxo-4-(trifluoromethyl)pyridin-1(2H)-yl)-1⁵,2⁶-dimethyl-6-oxo-3-oxa-7-aza-1,4(1,3),2(1,2)-tribenzenacyclooctaphane-8-yl)acetate (150 mg, 33.4%) as a yellow solid. LC-MS: (ES+H, m/z): [M+H]⁺=710.2. ¹H NMR (400 MHz, CD₃OD) δ 7.39-7.28 (m, 3H), 7.28-7.21 (m, 2H), 7.06 (d, J=7.5 Hz, 1H), 7.01 (dd, J=7.6, 1.7 Hz, 1H), 6.86 (dd, J=7.0, 2.2 Hz, 1H), 6.76 (s, 1H), 6.48 (s, 1H), 6.25 (dd, J=6.4, 2.2 Hz, 1H), 6.04 (d, J=2.1 Hz, 1H), 5.59 (t, J=7.8 Hz, 1H), 4.92-4.85 (m, 1H), 4.02-3.97 (m, 3H), 3.36-3.26 (m, 2H), 2.96-2.89 (m, 2H), 2.73 (dd, J=15.5, 8.1 Hz, 1H), 2.60 (dd, J=15.4, 7.5 Hz, 1H), 2.47-2.36 (m, 2H), 2.32 (q, J=6.9, 6.3 Hz, 2H), 2.23 (d, J=2.0 Hz, 3H), 1.88 (s, 3H), 1.13 (t, J=7.1 Hz, 3H).

### Step 3: Preparation of rel-2-((5S,8S)-1⁴-fluoro-5-(5-(2-(3-fluoroazetidin-1-yl)ethyl)-2-oxo-4-(trifluoromethyl)pyridin-1(2H)-yl)-1⁵,2⁶-dimethyl-6-oxo-3-oxa-7-aza-1,4(1,3),2(1,2)-tribenzenacyclooctaphane-8-yl)acetic acid

To a stirred solution of ethyl 2-((8S)-1⁴-fluoro-5-(5-(2-(3-fluoroazetidin-1-yl)ethyl)-2-oxo-4-(trifluoromethyl)pyridin-1(2H)-yl)-1⁵,2⁶-dimethyl-6-oxo-3-oxa-7-aza-1,4(1,3),2(1,2)-tribenzenacyclooctaphane-8-yl)acetate (70 mg, 0.10 mmol, 1.00 equiv) in THF (2 mL) was added LiOH·H₂O (20 mg, 0.49 mmol, 5.00 equiv) in H₂O (0.5 mL) dropwise at room temperature under nitrogen atmosphere. The resulting mixture was stirred for 3 h at room temperature under nitrogen atmosphere. The reaction was monitored by LCMS. The residue was neutralized to pH 7 with HCl (2M, aq.). The resulting mixture was concentrated under reduced pressure. The residue was purified by reverse flash chromatography (Column: XBridge Shield RP18 OBD Column, 50*250 mm, 10 μm; Mobile Phase A: Water (10 mmol/L HCO₂H), Mobile Phase B: MeCN; Flow rate: 100 mL/min; Gradient: 20% B to 60% B in 30 min, 45% B; wavelength: 254/220 nm; RT1 (min): 29)) to afford rel-2-((5S,8S)-1⁴-fluoro-5-(5-(2-(3-fluoroazetidin-1-yl)ethyl)-2-oxo-4-(trifluoromethyl)pyridin-1(2H)-yl)-1⁵,2⁶-dimethyl-6-oxo-3-oxa-7-aza-1,4(1,3),2(1,2)-tribenzenacyclooctaphane-8-yl)acetic acid (30.9 mg, 45.9%) as a white solid. LC-MS: (ES+H, m/z): [M+H]⁺=682.20. ¹⁹F NMR (377 MHz, CD₃OD) δ −64.98, −126.96, −180.56. ¹H NMR (400 MHz, CD₃OD) δ7.47-7.31 (m, 5H), 7.13 (dd, J=21.3, 7.5 Hz, 2H), 6.97-6.92 (m, 1H), 6.87 (s, 1H), 6.64 (s, 1H), 6.38 (dd, J=6.4, 2.2 Hz, 1H), 6.16 (t, J=2.1 Hz, 1H), 5.69 (t, J=7.3 Hz, 1H), 5.12-4.93 (m, 1H), 3.66-3.61 (m, 2H), 3.34 (d, J=4.9 Hz, 1H), 3.28-3.22 (m, 1H), 2.73-2.68 (m, 4H), 2.51-2.47 (m, 2H), 2.33 (d, J=1.8 Hz, 3H), 1.99 (s, 3H).

### Example 2

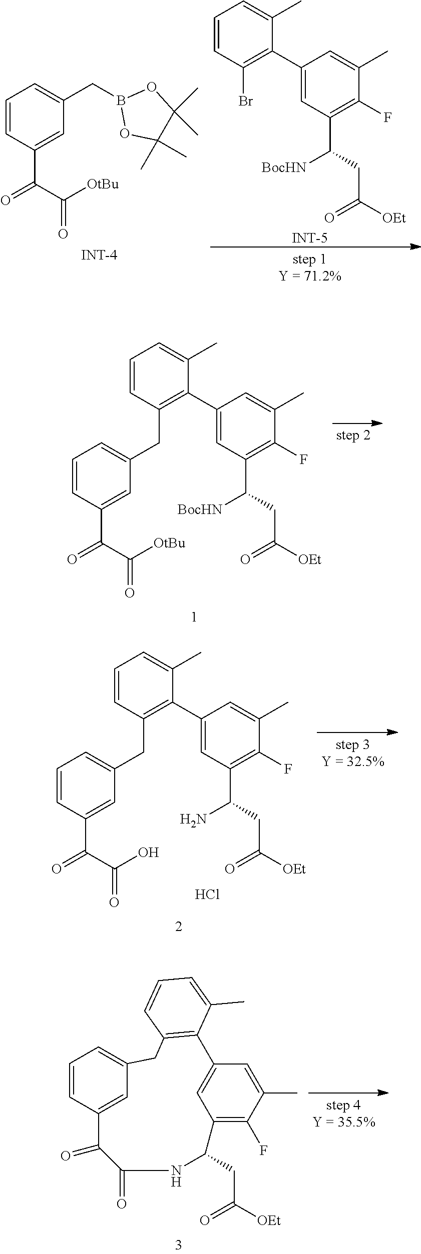

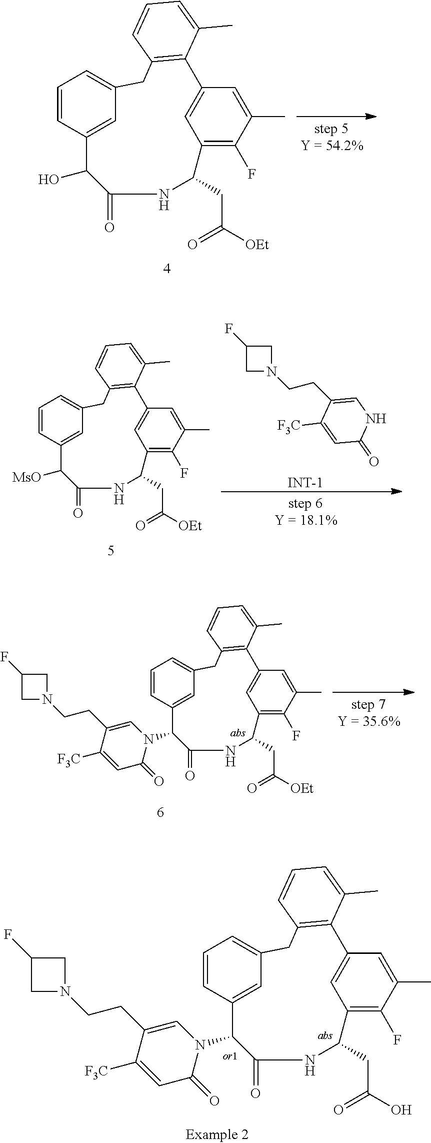 [SMILES: CCOC(=O)C[C@@H]1NC(=O)C(O)c2cccc(c2)Cc2cccc(C)c2-c2cc(C)c(F)c1c2 ; CCOC(=O)C[C@@H]1NC(=O)C(OS(C)(=O)=O)c2cccc(c2)Cc2cccc(C)c2-c2cc(C)c(F)c1c2 ; O=c1cc(C(F)(F)F)c(CCN2CC(F)C2)c[nH]1 ; "INT-1" ; CCOC(=O)C[C@@H]1NC(=O)[C@H](n2cc(CCN3CC(F)C3)c(C(F)(F)F)cc2=O)c2cccc(c2)Cc2cccc(C)c2-c2cc(C)c(F)c1c2 ; Cc1cc2cc(c1F)[C@H](CC(=O)O)NC(=O)[C@H](n1cc(CCN3CC(F)C3)c(C(F)(F)F)cc1=O)c1cccc(c1)Cc1cccc(C)c1-2]

### Step 1: Preparation of ethyl (3S)-3-[2′-({3-[2-(tert-butoxy)-2-oxoacetyl]phenyl}methyl)-4-fluoro-5,6′-dimethyl-[1,1′-biphenyl]-3-yl]-3-[(tert-butoxycarbonyl)amino]propanoate

To a stirred solution of tert-butyl 2-oxo-2-{3-[(4,4,5,5-tetramethyl-1,3,2-dioxaborolan-2-yl)methyl]phenyl}acetate (2.10 g, 6.06 mmol, 1.20 equiv.) and ethyl (3S)-3-{2′-bromo-4-fluoro-5,6′-dimethyl-[1,1′-biphenyl]-3-yl}-3-[(tert-butoxycarbonyl)amino]propanoate (2.50 g, 5.05 mmol, 1.00 equiv.) and K₂CO₃ (2.10 g, 15.17 mmol, 3.00 equiv.) in dioxane (20 mL) and H₂O (1 mL) was added XPhos Pd G3 (428 mg, 0.50 mmol, 0.10 equiv.) and XPhos (482 mg, 1.01 mmol, 0.20 equiv.) at room temperature under nitrogen atmosphere. The resulting mixture was stirred for 3 h at 100° C. under nitrogen atmosphere. The reaction was monitored by LCMS. The resulting mixture was concentrated under reduced pressure. The residue was purified by silica gel column chromatography, eluted with PE/EA (0% to 20% gradient in 30 min) to afford ethyl (3S)-3-[2′-({3-[2-(tert-butoxy)-2-oxoacetyl]phenyl}methyl)-4-fluoro-5,6′-dimethyl-[1,1′-biphenyl]-3-yl]-3-[(tert-butoxycarbonyl)amino]propanoate (2.40 g, 71.2%) as a white solid. LC-MS: (ES−H, m/z): [M−H]⁻=632.3. ¹H NMR (400 MHz, DMSO-d₆) δ 7.68 (t, J=8.1 Hz, 1H), 7.51-7.41 (m, 2H), 7.36-7.28 (m, 2H), 7.25 (dd, J=7.4, 2.0 Hz, 2H), 7.17 (t, J=8.5 Hz, 1H), 7.01 (ddd, J=30.4, 6.7, 2.2 Hz, 1H), 6.60-6.54 (m, 1H), 5.27 (dq, J=15.3, 8.4, 7.8 Hz, 1H), 4.11-4.04 (m, 2H), 3.90-3.79 (m, 2H), 2.77-2.62 (m, 2H), 2.13 (s, 3H), 1.98 (d, J=13.2 Hz, 3H), 1.56 (d, J=2.1 Hz, 9H), 1.38 (d, J=6.6 Hz, 9H), 1.20-1.15 (m, 3H).

### Step 2: Preparation of [3-({3′-[(1S)-1-amino-3-ethoxy-3-oxopropyl]-4′-fluoro-5′,6-dimethyl-[1,1′-biphenyl]-2-yl}methyl)phenyl](oxo)acetic acid hydrochloride

To a stirred solution of ethyl (3S)-3-[2′-({3-[2-(tert-butoxy)-2-oxoacetyl]phenyl}methyl)-4-fluoro-5,6′-dimethyl-[1,1′-biphenyl]-3-yl]-3-[(tert-butoxycarbonyl)amino]propanoate (1.20 g, 1.89 mmol, 1.00 equiv.) in DCM (5 mL) was added HCl in 1,4-dioxane (5 mL, 4M) at room temperature under nitrogen atmosphere. The resulting mixture was stirred for 2 h at room temperature under nitrogen atmosphere. The reaction was monitored by LCMS. The resulting mixture was concentrated under reduced pressure to afford crude [3-({3′-[(1S)-1-amino-3-ethoxy-3-oxopropyl]-4′-fluoro-5′,6-dimethyl-[1,1′-biphenyl]-2-yl}methyl)phenyl](oxo)acetic acid hydrochloride (1.00 g) which was used in the next step directly without further purification. LC-MS: (ES+H, m/z): [M+H]⁺=478.2.

### Step 3: Preparation of ethyl 2-[(17S)-19-fluoro-3,20-dimethyl-14,15-dioxo-16-azatetracyclo[16.3.1.1\hat{ }{9,13}.0\hat{ }{2,7}]tricosa-1(22),2(7),3,5,9,11,13(23),18,20-nonaen-17-yl]acetate

To a stirred solution of [3-({3′-[(1S)-1-amino-3-ethoxy-3-oxopropyl]-4′-fluoro-5′,6-dimethyl-[1,1′-biphenyl]-2-yl}methyl)phenyl](oxo)acetic acid hydrochloride (1.00 g, 2.09 mmol, 1.00 equiv, assumed 100% yield) and TCFH (1.18 g, 4.18 mmol, 2.00 equiv.) in MeCN (15 mL) was added NMI (0.86 g, 10.47 mmol, 5.00 equiv.) dropwise at room temperature under nitrogen atmosphere. The resulting mixture was stirred for 3 h at room temperature under nitrogen atmosphere. The reaction was monitored by LCMS. The resulting mixture was extracted with EtOAc (3×100 mL). The combined organic layers were washed with brine (3×30 mL), dried over anhydrous Na₂SO₄, filtered and concentrated. The residue was purified by silica gel column chromatography, eluted with PE/EA (0% to 20% gradient in 30 min) to afford ethyl 2-[(17S)-19-fluoro-3,20-dimethyl-14,15-dioxo-16-azatetracyclo[16.3.1.1\hat{ }{9,13}.0\hat{ }{2,7}]tricosa-1(22),2(7),3,5,9,11,13(23),18,20-nonaen-17-yl]acetate (330 mg, 32.5%) as a yellow solid. LC-MS: (ES+H, m/z): [M+H]⁺=460.2. ¹H NMR (300 MHz, DMSO-d₆) δ 9.46 (d, J=8.4 Hz, 1H), 7.86 (d, J=7.6 Hz, 1H), 7.73-7.66 (m, 1H), 7.55-7.45 (m, 2H), 7.27-7.11 (m, 4H), 6.82 (dd, J=6.7, 2.2 Hz, 1H), 6.54 (d, J=1.8 Hz, 1H), 5.67 (q, J=7.9 Hz, 1H), 4.06 (ddd, J=11.0, 7.8, 3.6 Hz, 2H), 3.80 (d, J=12.7 Hz, 1H), 2.90-2.79 (m, 2H), 2.36 (d, J=1.9 Hz, 3H), 1.92 (s, 3H), 1.14 (t, J=7.1 Hz, 3H).

### Step 4: Preparation of ethyl 2-[(17S)-19-fluoro-14-hydroxy-3,20-dimethyl-15-oxo-16-azatetracyclo[16.3.1.1\hat{ }{9,13}.0\hat{ }{2,7}]tricosa-1(22),2(7),3,5,9,11,13(23),18,20-nonaen-17-yl]acetate

To a stirred solution of ethyl 2-[(17S)-19-fluoro-3,20-dimethyl-14,15-dioxo-16-azatetracyclo[16.3.1.1\hat{ }{9,13}.0\hat{ }{2,7}]tricosa-1(22),2(7),3,5,9,11,13(23),18,20-nonaen-17-yl]acetate (320 mg, 0.69 mmol, 1.00 equiv.) in THF (3 mL) was added NaBH₄ (23 mg, 0.62 mmol, 0.90 equiv.) at room temperature under nitrogen atmosphere. The resulting mixture was stirred for 2 h at room temperature under nitrogen atmosphere. The reaction was monitored by LCMS. The reaction was quenched by the addition of water (0.5 mL) at 0° C., and extracted with EtOAc (3×50 mL). The combined organic layers were washed with brine (3×15 mL), dried over anhydrous Na₂SO₄, filtered and concentrated. The residue was purified by silica gel column chromatography, eluted with PE/EA (0% to 30% gradient in 40 min) to afford ethyl 2-[(17S)-19-fluoro-14-hydroxy-3,20-dimethyl-15-oxo-16-azatetracyclo[16.3.1.1\hat{ }{9,13}.0\hat{ }{2,7}]tricosa-1(22),2(7),3,5,9,11,13(23),18,20-nonaen-17-yl]acetate (120 mg, 35.5%) as a white solid. LC-MS: (ES+H, m/z): [M+H]⁺=462.2. ¹H NMR (300 MHz, DMSO-d₆) δ 8.32 (d, J=8.0 Hz, 1H), 7.54 (d, J=7.5 Hz, 1H), 7.44-7.12 (m, 5H), 7.02 (d, J=6.9 Hz, 1H), 6.39 (d, J=5.5 Hz, 1H), 5.84 (d, J=13.4 Hz, 1H), 5.65 (q, J=8.1 Hz, 1H), 5.18 (d, J=7.4 Hz, 1H), 4.92 (d, J=7.4 Hz, 1H), 4.09 (qt, J=7.1, 3.9 Hz, 2H), 3.80 (d, J=12.7 Hz, 1H), 2.89-2.65 (m, 2H), 2.38 (d, J=1.9 Hz, 3H), 1.90 (s, 3H), 1.16 (t, J=7.1 Hz, 3H).

### Step 5: Preparation of ethyl 2-[(17S)-19-fluoro-14-(methanesulfonyloxy)-3,20-dimethyl-15-oxo-16-azatetracyclo[16.3.1.1\hat{ }{9,13}.0\hat{ }{2,7}]tricosa-1(22),2(7),3,5,9,11,13(23),18,20-nonaen-17-yl]acetate

To a stirred solution of ethyl 2-[(17S)-19-fluoro-14-hydroxy-3,20-dimethyl-15-oxo-16-azatetracyclo[16.3.1.1\hat{ }{9,13}.0\hat{ }{2,7}]tricosa-1(22),2(7),3,5,9,11,13(23),18,20-nonaen-17-yl]acetate (120 mg, 0.26 mmol, 1.00 equiv.) and Et₃N (78 mg, 0.78 mmol, 3.00 equiv.) in DCM (2 mL) was added MsCl (44.67 mg, 0.39 mmol, 1.50 equiv.) at 0° C. under nitrogen atmosphere. The resulting mixture was stirred for 3 h at room temperature under nitrogen atmosphere and then concentrated. The residue was purified by silica gel column chromatography, eluted with PE/EA (0% to 30% gradient in 40 min) to afford ethyl 2-[(17S)-19-fluoro-14-(methanesulfonyloxy)-3,20-dimethyl-15-oxo-16-azatetracyclo[16.3.1.1\hat{ }{9,13}.0\hat{ }{2,7}]tricosa-1(22),2(7),3,5,9,11,13(23),18,20-nonaen-17-yl]acetate (80 mg, 54.2%) as a white solid. LC-MS: (ES+H, m/z): [M+H]⁺=540.2. ¹H NMR (300 MHz, DMSO-d₆) δ 8.62 (d, J=7.5 Hz, 1H), 7.47 (dd, J=19.7, 7.3 Hz, 2H), 7.29 (m, 3H), 7.11 (d, J=7.5 Hz, 1H), 6.98 (d, J=6.6 Hz, 1H), 6.29 (dd, J=6.7, 2.2 Hz, 1H), 5.90 (d, J=1.8 Hz, 2H), 5.56 (q, J=7.7 Hz, 1H), 4.11-3.95 (m, 2H), 3.78 (d, J=12.9 Hz, 1H), 3.28 (d, J=12.9 Hz, 1H), 3.24 (s, 3H), 2.86-2.62 (m, 2H), 2.33 (d, J=1.9 Hz, 3H), 1.84 (s, 3H), 1.10 (t, J=7.1 Hz, 3H).

### Step 6: Preparation of ethyl 2-[(17S)-19-fluoro-14-{5-[2-(3-fluoroazetidin-1-yl)ethyl]-2-oxo-4-(trifluoromethyl)pyridin-1-yl}-3,20-dimethyl-15-oxo-16-azatetracyclo[16.3.1.1\hat{ }{9,13}.0\hat{ }{2,7}]tricosa-1(22),2(7),3,5,9,11,13(23),18,20-nonaen-17-yl]acetate

To a stirred solution of ethyl 2-[(17S)-19-fluoro-14-(methanesulfonyloxy)-3,20-dimethyl-15-oxo-16-azatetracyclo[16.3.1.1\hat{ }{9,13}.0\hat{ }{2,7}]tricosa-1(22),2(7),3,5,9,11,13(23),18,20-nonaen-17-yl]acetate (80 mg, 0.14 mmol, 1.00 equiv.) and 5-[2-(3-fluoroazetidin-1-yl)ethyl]-4-(trifluoromethyl)-1H-pyridin-2-one (39.17 mg, 0.14 mmol, 1.00 equiv.) in MeCN (2 mL) was added K₂CO₃ (51.22 mg, 0.37 mmol, 2.50 equiv.) at room temperature under nitrogen atmosphere. The resulting mixture was stirred for 4 h at 80° C. under nitrogen atmosphere. The reaction was monitored by LCMS. The resulting mixture was concentrated under reduced pressure. The residue was dissolved in DMSO (1.5 mL). The residue was purified by reversed-phase flash chromatography (C18; mobile phase, MeCN in water (0.1% NH₃H₂O), 0% to 80% gradient in 50 min; detector, UV 254 nm) to provide ethyl 2-[(17S)-19-fluoro-14-{5-[2-(3-fluoroazetidin-1-yl)ethyl]-2-oxo-4-(trifluoromethyl)pyridin-1-yl}-3,20-dimethyl-15-oxo-16-azatetracyclo[16.3.1.1\hat{ }{9,13}.0\hat{ }{2,7}]tricosa-1(22),2(7),3,5,9,11,13(23),18,20-nonaen-17-yl]acetate (20 mg, 18.1%) as a white solid. LC-MS: (ES+H, m/z): [M+H]⁺=708.3. ¹H NMR (300 MHz, CD₃OD) δ 7.69-7.40 (m, 4H), 7.33 (t, J=7.7 Hz, 1H), 7.23 (t, J=7.6 Hz, 1H), 7.16-7.05 (m, 2H), 7.04-6.95 (m, 1H), 6.89 (s, 1H), 6.69 (s, 1H), 6.43 (dd, J=6.5, 2.2 Hz, 1H), 6.25 (t, J=1.8 Hz, 1H), 5.75 (dd, J=8.4, 7.3 Hz, 1H), 5.30 (s, 1H), 5.18 (d, J=5.7 Hz, 1H), 4.25-4.00 (m, 4H), 3.93-3.71 (m, 2H), 3.66-3.44 (m, 2H), 2.94-2.65 (m, 5H), 2.51 (h, J=8.3, 7.7 Hz, 1H), 2.37 (d, J=2.0 Hz, 2H), 2.26 (d, J=2.2 Hz, 3H), 1.91 (s, 2H), 1.19 (td, J=7.1, 1.4 Hz, 6H).

### Step 7: Preparation of [(14R*,17S)-19-fluoro-14-{5-[2-(3-fluoroazetidin-1-yl)ethyl]-2-oxo-4-(trifluoromethyl)pyridin-1-yl}-3,20-dimethyl-15-oxo-16-azatetracyclo[16.3.1.1\hat{ }{9,13}.0\hat{ }{2,7}]tricosa-1(22),2(7),3,5,9,11,13(23),18,20-nonaen-17-yl]acetic acid

To a stirred solution of ethyl 2-[(14R*,17S)-19-fluoro-14-{5-[2-(3-fluoroazetidin-1-yl)ethyl]-2-oxo-4-(trifluoromethyl)pyridin-1-yl}-3,20-dimethyl-15-oxo-16-azatetracyclo[16.3.1.1\hat{ }{9,13}.0\hat{ }{2,7}]tricosa-1(22),2(7),3,5,9,11,13(23),18,20-nonaen-17-yl]acetate (5.00 mg, 0.007 mmol, 1.00 equiv.) and H₂O (0.25 mL) in THF (1 mL) was added LiOH (1.00 mg, 0.04 mmol, 6.00 equiv.) at room temperature under nitrogen atmosphere. The resulting mixture was stirred for 2 h at room temperature under nitrogen atmosphere and then concentrated. The residue was dissolved in DMSO (1 mL) and the crude product was purified by Prep-HPLC (Column: XSelect CSH Prep C18 OBD Column, 30*150 mm, 5 μm; Mobile Phase A: Water (0.1% HCO₂H), Mobile Phase B: MeCN; Flow rate: 60 mL/min; Gradient: 20% B to 40% B in 8 min; wavelength: 220 nm; RT1 (min): 7.68) to afford [(14R*,17S)-19-fluoro-14-{5-[2-(3-fluoroazetidin-1-yl)ethyl]-2-oxo-4-(trifluoromethyl)pyridin-1-yl}-3,20-dimethyl-15-oxo-16-azatetracyclo[16.3.1.1\hat{ }{9,13}.0\hat{ }{2,7}]tricosa-1(22),2(7),3,5,9,11,13(23),18,20-nonaen-17-yl]acetic acid (1.8 mg, 35.6%). LC-MS: (ES+H, m/z): [M+H]⁺=680.20. ¹⁹F NMR (282 MHz, CD₃OD) δ −66.49, −129.52, −182.17. ¹H NMR (300 MHz, CD₃OD) δ 7.50 (dd, J=17.1, 7.6 Hz, 2H), 7.33 (t, J=7.6 Hz, 1H), 7.22 (t, J=7.5 Hz, 1H), 7.10 (dd, J=15.8, 8.6 Hz, 3H), 6.96 (d, J=6.1 Hz, 1H), 6.87 (s, 1H), 6.70 (s, 1H), 6.45 (s, 1H), 6.26 (s, 1H), 5.75 (s, 1H), 5.07-5.04 (m, 1H), 4.83-4.75 (m, 1H), 3.82 (d, J=12.9 Hz, 1H), 3.56-3.52 (m, 3H), 3.27-3.19 (m, 2H), 2.84-2.53 (m, 4H), 2.44 (t, J=7.2 Hz, 2H), 2.36 (s, 3H), 1.90 (s, 3H).

### Example 3

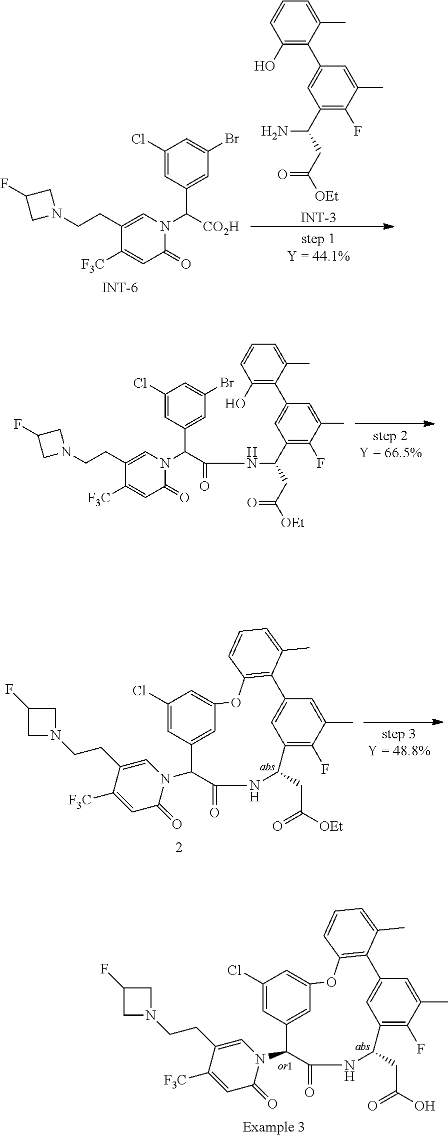

### Step 1: Preparation of ethyl (3S)-3-[2-(3-bromo-5-chlorophenyl)-2-{5-[2-(3-fluoroazetidin-1-yl)ethyl]-2-oxo-4-(trifluoromethyl)pyridin-1-yl}acetamido]-3-{4-fluoro-2′-hydroxy-5,6′-dimethyl-[1,1′-biphenyl]-3-yl}propanoate

To a stirred mixture of (3-bromo-5-chlorophenyl)({5-[2-(3-fluoroazetidin-1-yl)ethyl]-2-oxo-4-(trifluoromethyl)pyridin-1-yl})acetic acid (400 mg, 0.78 mmol, 1.00 equiv) and HOBT (137 mg, 1.01 mmol, 1.30 equiv) in MeCN (10 mL) were added EDCI (224 mg, 1.17 mmol, 1.50 equiv) at room temperature under nitrogen atmosphere. The resulting mixture was stirred for 1 h at room temperature under nitrogen atmosphere. To the above mixture was added ethyl (3S)-3-amino-3-{4-fluoro-2′-hydroxy-5,6′-dimethyl-[1,1′-biphenyl]-3-yl}propanoate (259 mg, 0.78 mmol, 1.00 equiv) and NEt₃ (474 mg, 4.69 mmol, 6.00 equiv) at 0° C. The resulting mixture was stirred overnight at 40° C. The reaction was monitored by LCMS. The resulting mixture was concentrated under reduced pressure and the crude product was purified by reversed-phase flash chromatography (C18; mobile phase, MeCN in water (0.1% NH₃H₂O), 30% to 80% gradient in 30 min; detector, UV 254 nm) to afford ethyl (3S)-3-[2-(3-bromo-5-chlorophenyl)-2-{5-[2-(3-fluoroazetidin-1-yl)ethyl]-2-oxo-4-(trifluoromethyl)pyridin-1-yl}acetamido]-3-{4-fluoro-2′-hydroxy-5,6′-dimethyl-[1,1′-biphenyl]-3-yl}propanoate (285 mg, 44.1%) as a colorless solid. LC-MS: (ES+H, m/z): [M+H]⁺=824.1.

### Step 2: Preparation of ethyl 2-[(17S)-11-chloro-19-fluoro-14-{5-[2-(3-fluoroazetidin-1-yl)ethyl]-2-oxo-4-(trifluoromethyl)pyridin-1-yl}-3,20-dimethyl-15-oxo-8-oxa-16-azatetracyclo[16.3.1.1\hat{ }{9,13}.0\hat{ }{2,7}]tricosa-1(22),2,4,6,9,11,13(23),18,20-nonaen-17-yl]acetate

To a stirred mixture of ethyl (3S)-3-[2-(3-bromo-5-chlorophenyl)-2-{5-[2-(3-fluoroazetidin-1-yl)ethyl]-2-oxo-4-(trifluoromethyl)pyridin-1-yl}acetamido]-3-{4-fluoro-2′-hydroxy-5,6′-dimethyl-[1,1′-biphenyl]-3-yl}propanoate (250 mg, 0.30 mmol, 1.00 equiv) and (1R,2R)—N1,N2-dimethylcyclohexane-1,2-diamine (51 mg, 0.36 mmol, 1.20 equiv) in dioxane (20 mL) were added K₂CO₃ (83 mg, 0.60 mmol, 2.00 equiv) and CuI (17 mg, 0.09 mmol, 0.30 equiv) at room temperature under nitrogen atmosphere. The resulting mixture was stirred for 3 h at 100° C. under nitrogen atmosphere. The reaction was monitored by LCMS. The resulting mixture was filtered and the filter cake was washed with EtOAc (3×100 mL). The filtrate was concentrated under reduced pressure. The residue was purified by reversed-phase flash chromatography (C18 gel; mobile phase, MeCN in water (0.10% NH₃H₂O), 20% to 50% gradient in 30 min; detector, UV 254 nm) to afford ethyl 2-[(17S)-11-chloro-19-fluoro-14-{5-[2-(3-fluoroazetidin-1-yl)ethyl]-2-oxo-4-(trifluoromethyl)pyridin-1-yl}-3,20-dimethyl-15-oxo-8-oxa-16-azatetracyclo[16.3.1.1\hat{ }{9,13}.0\hat{ }{2,7}]tricosa-1(22),2,4,6,9,11,13(23),18,20-nonaen-17-yl]acetate (150 mg, 66.5%) as a white solid. LC-MS: (ES+H, m/z): [M+H]⁺=744.2. ¹H NMR (300 MHz, DMSO-d₆) δ 8.93 (d, J=7.3 Hz, 1H), 7.70 (d, J=8.0 Hz, 1H), 7.60 (t, J=2.0 Hz, 1H), 7.41 (t, J=7.9 Hz, 1H), 7.34 (t, J=1.7 Hz, 1H), 7.23 (d, J=7.5 Hz, 1H), 7.19 (s, 1H), 7.02 (dd, J=7.2, 2.2 Hz, 1H), 6.81 (s, 1H), 6.39 (d, J=3.1 Hz, 2H), 5.93 (t, J=1.9 Hz, 1H), 5.48 (q, J=7.6 Hz, 1H), 5.03-4.73 (m, 1H), 4.13-3.97 (m, 2H), 3.25 (dq, J=14.1, 6.9 Hz, 2H), 2.90 (dt, J=8.4, 4.2 Hz, 1H), 2.82 (td, J=8.6, 3.8 Hz, 2H), 2.73 (d, J=6.7 Hz, 1H), 2.43-2.36 (m, 2H), 2.34 (s, 2H), 2.28 (d, J=1.8 Hz, 3H), 1.96 (s, 3H), 1.12 (t, J=7.1 Hz, 3H). ¹⁹F NMR (282 MHz, DMSO-d₆) δ −62.67, −125.76, −176.69.

### Step 3: Preparation of [(17S)-11-chloro-19-fluoro-14-{5-[2-(3-fluoroazetidin-1-yl)ethyl]-2-oxo-4-(trifluoromethyl)pyridin-1-yl}-3,20-dimethyl-15-oxo-8-oxa-16-azatetracyclo[16.3.1.1\hat{ }{9,13}.0\hat{ }{2,7}]tricosa-1(22),2,4,6,9,11,13(23),18,20-nonaen-17-yl]acetic acid

To a stirred mixture of ethyl 2-[(17S)-11-chloro-19-fluoro-14-{5-[2-(3-fluoroazetidin-1-yl)ethyl]-2-oxo-4-(trifluoromethyl)pyridin-1-yl}-3,20-dimethyl-15-oxo-8-oxa-16-azatetracyclo[16.3.1.1\hat{ }{9,13}.0\hat{ }{2,7}]tricosa-1(22),2,4,6,9,11,13(23),18,20-nonaen-17-yl]acetate (120 mg, 0.16 mmol, 1.00 equiv) in THF (3 mL) was added LiOH·H₂O (8 mg, 0.32 mmol, 2.00 equiv) in H₂O (1.5 mL) dropwise at 0° C. under nitrogen atmosphere. The resulting mixture was stirred for 2 h at room temperature under nitrogen atmosphere. The reaction was monitored by LCMS. The mixture was acidified to pH 5 with cone. HCl and the precipitated solids were collected by filtration and washed with water (3×10 mL). The residue was purified by reversed-phase flash chromatography (C18; mobile phase, MeCN in water (0.1% NH₃H₂O), 50% to 80% gradient in 10 min; detector, UV 220 nm) to afford [(17S)-11-chloro-19-fluoro-14-{5-[2-(3-fluoroazetidin-1-yl)ethyl]-2-oxo-4-(trifluoromethyl)pyridin-1-yl}-3,20-dimethyl-15-oxo-8-oxa-16-azatetracyclo[16.3.1.1\hat{ }{9,13}.0\hat{ }{2,7}]tricosa-1(22),2,4,6,9,11,13(23),18,20-nonaen-17-yl]acetic acid (56.4 mg, 48.8%). LC-MS: (ES+H, m/z): [M+H]⁺=716.2. ¹H NMR (400 MHz, DMSO-d₆) δ 12.48 (s, 1H), 8.98 (d, J=7.2 Hz, 1H), 7.69 (d, J=8.0 Hz, 1H), 7.59 (t, J=2.0 Hz, 1H), 7.40 (t, J=7.9 Hz, 1H), 7.31 (d, J=1.7 Hz, 1H), 7.24-7.17 (m, 2H), 7.00 (dd, J=7.0, 2.2 Hz, 1H), 6.80 (s, 1H), 6.41 (s, 1H), 6.38 (dd, J=6.4, 2.2 Hz, 1H), 5.93 (t, J=1.8 Hz, 1H), 5.46 (dt, J=9.1, 6.5 Hz, 1H), 4.87 (dp, J=57.8, 5.3 Hz, 1H), 3.30-3.17 (m, 2H), 2.86 (ddt, J=24.1, 8.6, 4.6 Hz, 2H), 2.74-2.55 (m, 2H), 2.40 (ddd, J=15.9, 9.3, 4.7 Hz, 2H), 2.33 (d, J=5.2 Hz, 2H), 2.28 (d, J=1.8 Hz, 3H), 1.95 (s, 3H). ¹⁹F NMR (377 MHz, DMSO-d₆) δ −62.58, −125.66, −176.97.

### Example 4

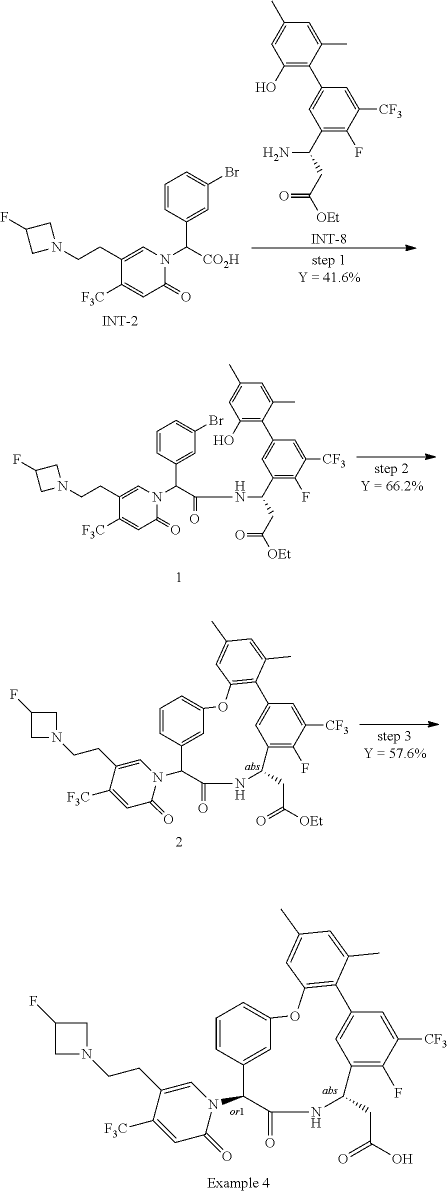

### Step 1: Preparation of ethyl (3S)-3-[2-(3-bromophenyl)-2-{5-[2-(3-fluoroazetidin-1-yl)ethyl]-2-oxo-4-(trifluoromethyl)pyridin-1-yl}acetamido]-3-[4-fluoro-2′-hydroxy-4′,6′-dimethyl-5-(trifluoromethyl)-[1,1′-biphenyl]-3-yl]propanoate

A solution of ethyl (3S)-3-amino-3-[4-fluoro-2′-hydroxy-4′,6′-dimethyl-5-(trifluoromethyl)-[1,1′-biphenyl]-3-yl]propanoate hydrochloride (427 mg, assumed 100% yield, 0.98 mmol, 1.00 equiv) and TEA (495 mg, 4.90 mmol, 5.00 equiv) in MeCN (10 mL) was stirred for 1 h at room temperature under nitrogen atmosphere. The resulting mixture was concentrated under reduced pressure. This resulted in ethyl (3S)-3-amino-3-[4-fluoro-2′-hydroxy-4′,6′-dimethyl-5-(trifluoromethyl)-[1,1′-biphenyl]-3-yl]propanoate. A solution of (3-bromophenyl)({5-[2-(3-fluoroazetidin-1-yl)ethyl]-2-oxo-4-(trifluoromethyl)pyridin-1-yl})acetic acid (514 mg, 1.07 mmol, 1.10 equiv), HOBT (172 mg, 1.27 mmol, 1.30 equiv) and EDCI (300 mg, 1.56 mmol, 1.60 equiv) in MeCN (10 mL) was stirred for 1 h at room temperature under nitrogen atmosphere. To the above mixture was added ethyl (3S)-3-amino-3-[4-fluoro-2′-hydroxy-4′,6′-dimethyl-5-(trifluoromethyl)-[1,1′-biphenyl]-3-yl]propanoate in MeCN (0.5 mL) dropwise over 5 min at 0° C. The resulting mixture was stirred for an additional 3 h at 30° C. The reaction was monitored by LCMS. Desired product could be detected by LCMS. The resulting mixture was concentrated under reduced pressure. The residue was purified by reversed-phase flash chromatography (C18; mobile phase, MeCN in water (0.10% NH₃H₂O), 30% to 70% gradient in 15 min; detector, UV 254 nm) to provide ethyl (3S)-3-[2-(3-bromophenyl)-2-{5-[2-(3-fluoroazetidin-1-yl)ethyl]-2-oxo-4-(trifluoromethyl)pyridin-1-yl}acetamido]-3-[4-fluoro-2′-hydroxy-4′,6′-dimethyl-5-(trifluoromethyl)-[1,1′-biphenyl]-3-yl]propanoate (350 mg, 41.6%) as light yellow solid. LC-MS: (ES+H, m/z): [M+H]⁺=860.15.

### Step 2: Preparation of ethyl 2-[(17S)-19-fluoro-14-{5-[2-(3-fluoroazetidin-1-yl)ethyl]-2-oxo-4-(trifluoromethyl)pyridin-1-yl}-3,5-dimethyl-15-oxo-20-(trifluoromethyl)-8-oxa-16-azatetracyclo[16.3.1.1\hat{ }{9,13}.0\hat{ }{2,7}]tricosa-1(22),2,4,6,9,11,13(23),18,20-nonaen-17-yl]acetate

To a stirred solution of ethyl (3S)-3-[2-(3-bromophenyl)-2-{5-[2-(3-fluoroazetidin-1-yl)ethyl]-2-oxo-4-(trifluoromethyl)pyridin-1-yl}acetamido]-3-[4-fluoro-2′-hydroxy-4′,6′-dimethyl-5-(trifluoromethyl)-[1,1′-biphenyl]-3-yl]propanoate (300 mg, 0.34 mmol, 1.00 equiv) in 1,4-dioxane (12 mL) were added CuI (33 mg, 0.17 mmol, 0.50 equiv), K₂CO₃ (96 mg, 0.69 mmol, 2.00 equiv) and (1R,2R)—N1,N2-dimethylcyclohexane-1,2-diamine (49 mg, 0.34 mmol, 1.00 equiv) at room temperature under nitrogen atmosphere. The resulting mixture was stirred overnight at 100° C. The reaction was monitored by LCMS. The mixture was allowed to cool down to room temperature. The resulting mixture was concentrated under reduced pressure. The residue was purified by reversed-phase flash chromatography (C18; mobile phase, MeCN in water (0.10% NH₃H₂O), 10% to 50% gradient in 10 min; detector, UV 254 nm) to afford ethyl 2-[(17S)-19-fluoro-14-{5-[2-(3-fluoroazetidin-1-yl)ethyl]-2-oxo-4-(trifluoromethyl)pyridin-1-yl}-3,5-dimethyl-15-oxo-20-(trifluoromethyl)-8-oxa-16-azatetracyclo[16.3.1.1\hat{ }{9,13}.0\hat{ }{2,7}]tricosa-1(22),2,4,6,9,11,13(23),18,20-nonaen-17-yl]acetate (180 mg, 66.2%) as light yellow solid. LC-MS: (ES+H, m/z): [M+H]⁺=778.3. ¹H NMR (300 MHz, CDCl₃) δ7.52-7.43 (m, 1H), 7.42-7.34 (m, 4H), 7.23 (d, J=1.7 Hz, 1H), 7.15-7.10 (m, 1H), 7.01 (dd, J=1.8, 0.9 Hz, 1H), 6.85 (s, 1H), 6.68 (q, J=2.6, 2.1 Hz, 2H), 6.37 (t, J=2.0 Hz, 1H), 5.83-5.74 (m, 1H), 5.03-4.74 (m, 1H), 4.12-3.91 (m, 2H), 3.41 (q, J=6.4 Hz, 2H), 3.06-2.74 (m, 4H), 2.61-2.44 (m, 2H), 2.46 (s, 3H), 2.39 (t, J=6.6 Hz, 2H), 2.01 (s, 1H), 1.93 (s, 3H), 1.12 (t, J=7.1 Hz, 3H). ¹⁹F NMR (282 MHz, CDCl₃) δ −61.04, −63.49, −122.11, −178.00.

### Step 3: Preparation of [(14R*,17S)-19-fluoro-14-{5-[2-(3-fluoroazetidin-1-yl)ethyl]-2-oxo-4-(trifluoromethyl)pyridin-1-yl}-3,5-dimethyl-15-oxo-20-(trifluoromethyl)-8-oxa-16-azatetracyclo[16.3.1.1\hat{ }{9,13}.0\hat{ }{2,7}]tricosa-1(21),2(7),3,5,9,11,13(23),18(22),19-nonaen-17-yl]acetic acid

To a stirred solution of ethyl 2-[(17S)-19-fluoro-14-{5-[2-(3-fluoroazetidin-1-yl)ethyl]-2-oxo-4-(trifluoromethyl)pyridin-1-yl}-3,5-dimethyl-15-oxo-20-(trifluoromethyl)-8-oxa-16-azatetracyclo[16.3.1.1\hat{ }{9,13}.0\hat{ }{2,7}]tricosa-1(22),2,4,6,9,11,13(23),18,20-nonaen-17-yl]acetate (150 mg, 0.19 mmol, 1.00 equiv) in THF (5 mL) and H₂O (1 mL) was added LiOH·H₂O (40 mg, 0.96 mmol, 5.00 equiv) at room temperature under nitrogen atmosphere. The resulting mixture was stirred for 1.5 h at room temperature. The reaction was monitored by LCMS. The mixture was acidified to pH 5 with conc. HCl. The resulting mixture was concentrated under reduced pressure. The residue was purified by reverse phase flash chromatography (Column: XSelect CSH Prep C18 OBD Column, 30*150 mm, 5 μm; Mobile Phase A: Water (0.1% HCO₂H), Mobile Phase B: MeCN; Flow rate: 60 mL/min; Gradient: 20% B to 40% B in 8.5 min; Wavelength: 220 nm; RT1 (min): 9.52)) to provide [(14R*,17S)-19-fluoro-14-{5-[2-(3-fluoroazetidin-1-yl)ethyl]-2-oxo-4-(trifluoromethyl)pyridin-1-yl}-3,5-dimethyl-15-oxo-20-(trifluoromethyl)-8-oxa-16-azatetracyclo[16.3.1.1\hat{ }{9,13}.0\hat{ }{2,7}]tricosa-1(21),2(7),3,5,9,11,13(23),18(22),19-nonaen-17-yl]acetic acid (83.3 mg, 57.6%). LC-MS: (ES+H, m/z): [M+H]⁺=750.2. ¹H NMR (400 MHz, CD₃OD) δ 7.51-7.40 (m, 2H), 7.36 (d, J=7.3 Hz, 2H), 7.32 (d, J=1.7 Hz, 1H), 7.16-7.12 (m, 1H), 7.03 (d, J=1.6 Hz, 1H), 6.88 (s, 1H), 6.83 (dd, J=6.1, 2.1 Hz, 1H), 6.62 (s, 1H), 6.09 (t, J=2.0 Hz, 1H), 5.68 (t, J=7.4 Hz, 1H), 5.12-4.90 (m, 1H), 3.73-7.61 (m, 2H), 3.29-3.27 (m, 1H), 3.36 (t, J=5.4 Hz, 1H), 2.84-2.68 (m, 4H), 2.65 (s, 1H), 2.57-7.44 (m, 2H), 2.43 (s, 3H), 1.94 (d, J=3.1 Hz, 3H). ¹⁹F NMR (282 MHz, CDCl₃) δ −62.50, −62.54, −64.96, −123.88, −123.91, −180.65

### Example 5

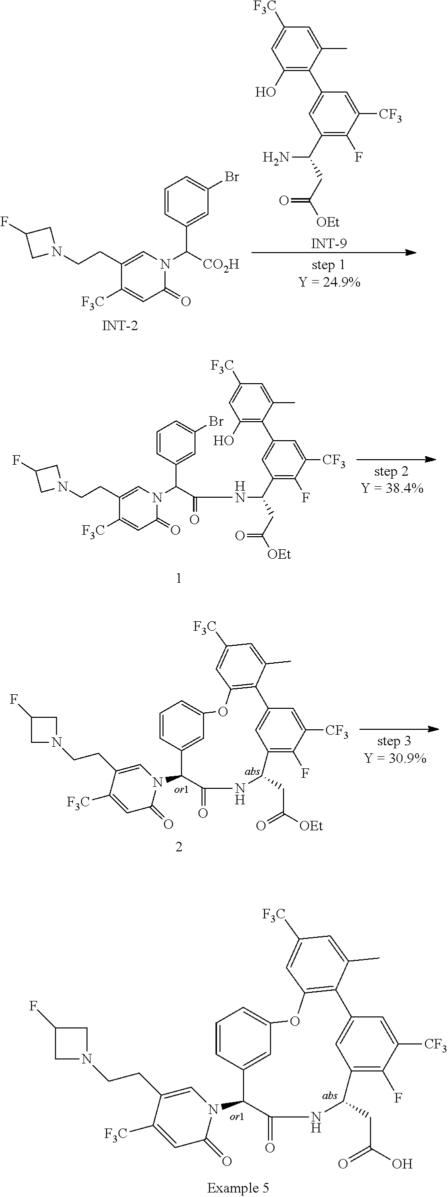

### Step 1: Preparation of ethyl (3S)-3-[2-(3-bromophenyl)-2-{5-[2-(3-fluoroazetidin-1-yl)ethyl]-2-oxo-4-(trifluoromethyl)pyridin-1-yl}acetamido]-3-[4-fluoro-2′-hydroxy-6′-methyl-4′,5-bis(trifluoromethyl)-[1,1′-biphenyl]-3-yl]propanoate

A solution of ethyl (3S)-3-amino-3-[4-fluoro-2′-hydroxy-6′-methyl-4′,5-bis(trifluoromethyl)-[1,1′-biphenyl]-3-yl]propanoate hydrochloride (400 mg, assumed 100% yield, 0.88 mmol, 1.00 equiv) and TEA (534 mg, 5.28 mmol, 6.00 equiv) in MeCN (5 mL) was stirred for 1 h at room temperature under nitrogen atmosphere. The resulting mixture was concentrated under reduced pressure to afford ethyl (3S)-3-amino-3-[4-fluoro-2′-hydroxy-6′-methyl-4′,5-bis(trifluoromethyl)-[1,1′-biphenyl]-3-yl]propanoate. A solution of (3-bromophenyl)({5-[2-(3-fluoroazetidin-1-yl)ethyl]-2-oxo-4-(trifluoromethyl)pyridin-1-yl})acetic acid (420 mg, 0.88 mmol, 1.00 equiv), HOBT (155 mg, 1.14 mmol, 1.30 equiv) and EDCI (270 mg, 1.41 mmol, 1.60 equiv) in MeCN (6 mL) was stirred for 1 h at room temperature under nitrogen atmosphere. To the above mixture was added ethyl (3S)-3-amino-3-[4-fluoro-2′-hydroxy-6′-methyl-4′,5-bis(trifluoromethyl)-[1,1′-biphenyl]-3-yl]propanoate in MeCN (4 mL) dropwise over 5 min at 0° C. The resulting mixture was stirred overnight at 30° C. The reaction was monitored by LCMS. The resulting mixture was concentrated under reduced pressure. The residue was purified by reversed-phase flash chromatography (column, C18; mobile phase, MeCN in water (0.1% NH₃·H₂O), 40% to 70% gradient in 20 min; detector, UV 220 nm) to provide ethyl (3S)-3-[2-(3-bromophenyl)-2-{5-[2-(3-fluoroazetidin-1-yl)ethyl]-2-oxo-4-(trifluoromethyl)pyridin-1-yl}acetamido]-3-[4-fluoro-2′-hydroxy-6′-methyl-4′,5-bis(trifluoromethyl)-[1,1′-biphenyl]-3-yl]propanoate (200 mg, 24.9%) as a colorless oil. LC-MS: (ES+H, m/z): [M+H]⁺=912.2.

### Step 2: Preparation of ethyl 2-[(17S)-19-fluoro-14-{5-[2-(3-fluoroazetidin-1-yl)ethyl]-2-oxo-4-(trifluoromethyl)pyridin-1-yl}-3-methyl-15-oxo-5,20-bis(trifluoromethyl)-8-oxa-16-azatetracyclo[16.3.1.1\hat{ }{9,13}.0\hat{ }{2,7}]tricosa-1(22),2,4,6,9,11,13(23),18,20-nonaen-17-yl]acetate

A mixture of ethyl (3S)-3-[2-(3-bromophenyl)-2-{5-[2-(3-fluoroazetidin-1-yl)ethyl]-2-oxo-4-(trifluoromethyl)pyridin-1-yl}acetamido]-3-[4-fluoro-2′-hydroxy-6′-methyl-4′,5-bis(trifluoromethyl)-[1,1′-biphenyl]-3-yl]propanoate (200 mg, 0.22 mmol, 1.00 equiv), CuI (21 mg, 0.11 mmol, 0.50 equiv), K₂CO₃ (61 mg, 0.44 mmol, 2.00 equiv) and (1R,2R)—N1,N2-dimethylcyclohexane-1,2-diamine (31 mg, 0.22 mmol, 1.00 equiv) in dioxane (10 mL) was stirred overnight at 100° C. under nitrogen atmosphere and then cooled to room temperature. The resulting mixture was concentrated under reduced pressure. The residue was purified by reversed-phase flash chromatography (C18; mobile phase, MeCN in water (0.1% NH₃·H₂O), 40% to 70% gradient in 20 min; detector, UV 220 nm) to provide ethyl 2-[(17S)-19-fluoro-14-{5-[2-(3-fluoroazetidin-1-yl)ethyl]-2-oxo-4-(trifluoromethyl)pyridin-1-yl}-3-methyl-15-oxo-5,20-bis(trifluoromethyl)-8-oxa-16-azatetracyclo[16.3.1.1\hat{ }{9,13}.0\hat{ }{2,7}]tricosa-1(22),2,4,6,9,11,13(23),18,20-nonaen-17-yl]acetate (70 mg, 38.4%) as a colorless oil. LC-MS: (ES+H, m/z): [M+H]⁺=832.3. ¹H NMR (400 MHz, CDCl₃) δ 7.74 (d, J=7.2 Hz, 1H), 7.60 (s, 1H), 7.41-7.32 (m, 2H), 7.31-7.22 (m, 3H), 7.08 (d, J=7.5 Hz, 1H), 6.73 (d, J=2.7 Hz, 1H), 6.68 (d, J=3.2 Hz, 2H), 6.26 (d, J=2.2 Hz, 1H), 5.70 (dd, J=10.3, 4.5 Hz, 1H), 4.90-4.68 (m, 1H), 4.00-3.86 (m, 2H), 3.31 (dd, J=13.3, 7.1 Hz, 2H), 2.93-2.73 (m, 4H), 2.53-2.23 (m, 5H), 1.96 (d, J=2.5 Hz, 2H), 1.06 (t, J=7.3, 2.3 Hz, 3H). ¹⁹F NMR (377 MHz, CDCl₃) δ −61.10, −61.13, −62.66, −63.85, −120.60, −178.28.

### Step 3: Preparation of [(14R*,17S)-19-fluoro-14-{5-[2-(3-fluoroazetidin-1-yl)ethyl]-2-oxo-4-(trifluoromethyl)pyridin-1-yl}-3-methyl-15-oxo-5,20-bis(trifluoromethyl)-8-oxa-16-azatetracyclo[16.3.1.1\hat{ }{9,13}.0\hat{ }{2,7}]tricosa-1(21),2(7),3,5,9,11,13(23),18(22),19-nonaen-17-yl]acetic acid

A mixture of ethyl 2-[(17S)-19-fluoro-14-{5-[2-(3-fluoroazetidin-1-yl)ethyl]-2-oxo-4-(trifluoromethyl)pyridin-1-yl}-3-methyl-15-oxo-5,20-bis(trifluoromethyl)-8-oxa-16-azatetracyclo[16.3.1.1\hat{ }{9,13}.0\hat{ }{2,7}]tricosa-1(22),2,4,6,9,11,13(23),18,20-nonaen-17-yl]acetate (70 mg, 0.08 mmol, 1.00 equiv) and LiOH·H₂O (14 mg, 0.34 mmol, 4.00 equiv) in THF (5 mL) and H₂O (1 mL) was stirred for 3 h at room temperature under nitrogen atmosphere. The reaction was monitored by LCMS. The mixture was acidified to pH 3 with HCl (aq. 1 M). The resulting mixture was concentrated under reduced pressure. The crude product (70 mg) was purified by Prep-HPLC (Column: Xselect CSH C18 OBD Column 30*150 mm, 5 μm; Mobile Phase A: Water (0.1% HCO₂H), Mobile Phase B: MeCN; Flow rate: 60 mL/min; Gradient: 30% B to 40% B in 8 min; Wavelength: 254 nm/220 nm; RT1 (min): 9.48) to afford [(14R*,17S)-19-fluoro-14-{5-[2-(3-fluoroazetidin-1-yl)ethyl]-2-oxo-4-(trifluoromethyl)pyridin-1-yl}-3-methyl-15-oxo-5,20-bis(trifluoromethyl)-8-oxa-16-azatetracyclo[16.3.1.1\hat{ }{9,13}.0\hat{ }{2,7}]tricosa-1(21),2(7),3,5,9,11,13(23),18(22),19-nonaen-17-yl]acetic acid (21.9 mg, ee %=95.9%, 30.9%). LC-MS: (ES+H, m/z): [M+H]⁺=804.20. ¹H NMR (300 MHz, CD₃OD) δ 7.88 (s, 1H), 7.57 (s, 1H), 7.50 (dd, J=3.7, 1.8 Hz, 3H), 7.37 (s, 1H), 7.20 (q, J=3.7, 3.2 Hz, 1H), 6.92 (d, J=9.8 Hz, 2H), 6.66 (s, 1H), 6.11 (s, 1H), 5.70 (t, J=6.9 Hz, 1H), 5.17-4.94 (m, 1H), 3.66 (s, 2H), 3.41-3.35 (m, 1H), 3.31-3.24 (m, 1H), 2.75 (t, J=12.6 Hz, 4H), 2.51 (q, J=7.6, 7.0 Hz, 2H), 2.09 (s, 3H). ¹⁹F NMR (282 MHz, CD₃OD) δ −62.53, −62.58, −64.05, −64.97, −122.48, −122.53, −180.59.

### Example 6

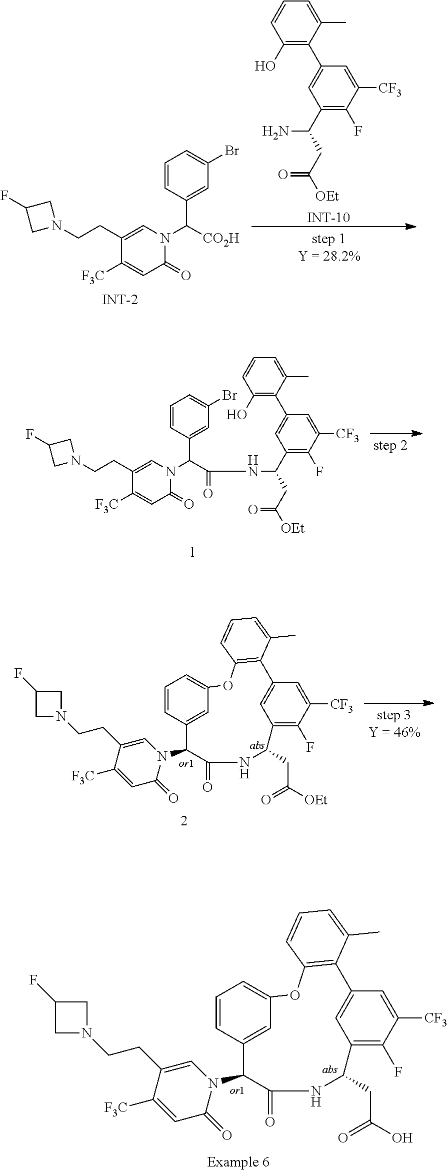

### Step 1: Preparation of ethyl (3S)-3-[2-(3-bromophenyl)-2-{5-[2-(3-fluoroazetidin-1-yl)ethyl]-2-oxo-4-(trifluoromethyl)pyridin-1-yl}acetamido]-3-[4-fluoro-2′-hydroxy-6′-methyl-5-(trifluoromethyl)-[1,1′-biphenyl]-3-yl]propanoate

A solution of (3-bromophenyl)({5-[2-(3-fluoroazetidin-1-yl)ethyl]-2-oxo-4-(trifluoromethyl)pyridin-1-yl})acetic acid (240 mg, 0.50 mmol, 1.00 equiv) and HOBt (88 mg, 0.65 mmol, 1.30 equiv), EDCI (144 mg, 0.75 mmol, 1.50 equiv) in CH₃CN (3 mL) was stirred for 1 h at room temperature under nitrogen atmosphere. To the above mixture was added ethyl (3S)-3-amino-3-[4-fluoro-2′-hydroxy-6′-methyl-5-(trifluoromethyl)-[1,1′-biphenyl]-3-yl]propanoate (251 mg, crude), Et₃N (152 mg, 1.50 mmol, 3.00 equiv) in CH₃CN (2 mL) dropwise over 5 min at room temperature. The resulting mixture was stirred for an additional 24 h at room temperature. The reaction was monitored by LCMS. The resulting mixture was diluted with EtOAc (150 mL). The residue was washed with water (2×20 mL), dried over anhydrous Na₂SO₄, filtered and concentrated. The residue was purified by reversed-phase flash chromatography (C18; mobile phase, CH₃CN in water (0.1% NH₃·H₂O), 0% to 70% gradient in 40 min; detector, UV 220 nm) to provide ethyl (3S)-3-[2-(3-bromophenyl)-2-{5-[2-(3-fluoroazetidin-1-yl)ethyl]-2-oxo-4-(trifluoromethyl)pyridin-1-yl}acetamido]-3-[4-fluoro-2′-hydroxy-6′-methyl-5-(trifluoromethyl)-[1,1′-biphenyl]-3-yl]propanoate (120 mg, 28.2%) as a yellow solid. LC-MS: (ES+H, m/z): [M+H]⁺=844.2.

### Step 2: Preparation of ethyl 2-((8S)-14-fluoro-5-(5-(2-(3-fluoroazetidin-1-yl)ethyl)-2-oxo-4-(trifluoromethyl)pyridin-1(2H)-yl)-26-methyl-6-oxo-15-(trifluoromethyl)-3-oxa-7-aza-1,4(1,3),2(1,2)-tribenzenacyclooctaphane-8-yl)acetate

To a stirred solution of ethyl (3S)-3-[2-(3-bromophenyl)-2-{5-[2-(3-fluoroazetidin-1-yl)ethyl]-2-oxo-4-(trifluoromethyl)pyridin-1-yl}acetamido]-3-[4-fluoro-2′-hydroxy-6′-methyl-5-(trifluoromethyl)-[1,1′-biphenyl]-3-yl]propanoate (100 mg, 0.12 mmol, 1.00 equiv) and CuI (12 mg, 0.06 mmol, 0.50 equiv) in dioxane (5 mL) was added (1R,2R)—N1,N2-dimethylcyclohexane-1,2-diamine (17 mg, 0.12 mmol, 1.00 equiv) and K₂CO₃ (33 mg, 0.24 mmol, 2.00 equiv) in portions at room temperature under nitrogen atmosphere. The resulting mixture was stirred overnight at 100° C. under nitrogen atmosphere and then cooled to room temperature. The resulting mixture was diluted with EtOAc (150 mL). The residue was washed with water (2×20 mL), dried over anhydrous Na₂SO₄, filtered and concentrated. The residue was purified by reversed-phase flash chromatography (C18; mobile phase, MeCN in water (0.1% NH₃·H₂O), 0% to 70% gradient in 40 min; detector, UV 220 nm) to provide ethyl 2-((8S)-14-fluoro-5-(5-(2-(3-fluoroazetidin-1-yl)ethyl)-2-oxo-4-(trifluoromethyl)pyridin-1(2H)-yl)-26-methyl-6-oxo-15-(trifluoromethyl)-3-oxa-7-aza-1,4(1,3),2(1,2)-tribenzenacyclooctaphane-8-yl)acetate (70 mg, crude) as a yellow solid. LC-MS: (ES+H, m/z): [M+H]⁺=764.2.

### Step 3: Preparation of rel-2-((5S,8S)-14-fluoro-5-(5-(2-(3-fluoroazetidin-1-yl)ethyl)-2-oxo-4-(trifluoromethyl)pyridin-1(2H)-yl)-26-methyl-6-oxo-15-(trifluoromethyl)-3-oxa-7-aza-1,4(1,3),2(1,2)-tribenzenacyclooctaphane-8-yl)acetic acid

To a stirred solution of ethyl 2-((8S)-14-fluoro-5-(5-(2-(3-fluoroazetidin-1-yl)ethyl)-2-oxo-4-(trifluoromethyl)pyridin-1(2H)-yl)-26-methyl-6-oxo-15-(trifluoromethyl)-3-oxa-7-aza-1,4(1,3),2(1,2)-tribenzenacyclooctaphane-8-yl)acetate (70 mg, crude) in THF (1 mL) were added LiOH·H₂O (19 mg, 0.46 mmol, 5.00 equiv) in H₂O (0.2 mL) dropwise at room temperature under nitrogen atmosphere. The resulting mixture was stirred for 3 h at room temperature under nitrogen atmosphere. The reaction was monitored by LCMS. The mixture was neutralized to pH 7 with HCl (aq. 1M). The residue was purified by reverse flash chromatography (Column: XBridge Shield RP18 OBD Column, 50*250 mm, 10 μm; Mobile Phase A: Water (10 mmol/L HCO₂H), Mobile Phase B: MeCN; Flow rate: 100 mL/min; Gradient: 20% B to 60% B in 40 min, 50% B; Wavelength: 254/220 nm; RT1 (min): 30)) to provide rel-2-((5S,8S)-14-fluoro-5-(5-(2-(3-fluoroazetidin-1-yl)ethyl)-2-oxo-4-(trifluoromethyl)pyridin-1(2H)-yl)-26-methyl-6-oxo-15-(trifluoromethyl)-3-oxa-7-aza-1,4(1,3),2(1,2)-tribenzenacyclooctaphane-8-yl)acetic acid (32.6 mg, 46.0%). LC-MS: (ES+H, m/z): [M+H]⁺=736.2. ¹H NMR (400 MHz, CD₃OD) δ 7.50-7.37 (m, 6H), 7.21-7.13 (m, 2H), 6.87-6.85 (m, 2H), 6.62 (s, 1H), 6.09 (s, 1H), 5.68 (t, J=7.3 Hz, 1H), 4.91-4.85 (m, 1H), 3.64-3.55 (m, 2H), 3.31-3.25 (m, 2H), 2.78-2.66 (m, 4H), 2.51-2.46 (m, 2H), 1.98 (s, 3H). ¹⁹F NMR (377 MHz, CD₃OD) δ −62.53, −64.97, −123.65, −180.57.

### Example 7

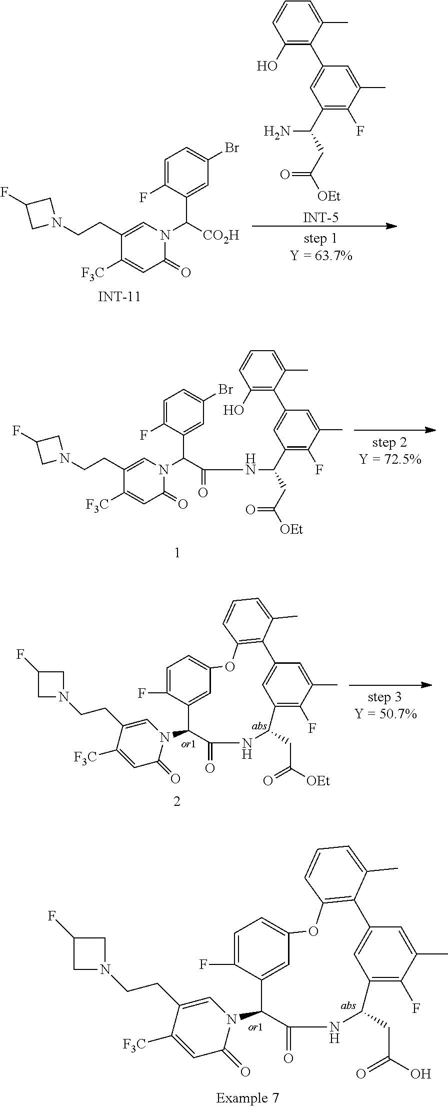

### Step 1: Preparation of ethyl (3S)-3-[2-(5-bromo-2-fluorophenyl)-2-{5-[2-(3-fluoroazetidin-1-yl)ethyl]-2-oxo-4-(trifluoromethyl)pyridin-1-yl}acetamido]-3-{4-fluoro-2′-hydroxy-5,6′-dimethyl-[1,1′-biphenyl]-3-yl}propanoate

To a stirred mixture of (5-bromo-2-fluorophenyl)({5-[2-(3-fluoroazetidin-1-yl)ethyl]-2-oxo-4-(trifluoromethyl)pyridin-1-yl})acetic acid (250 mg, 0.50 mmol, 1.00 equiv) in CH₃CN (5 mL) were added HOBT (95 mg, 0.70 mmol, 1.40 equiv) and EDCI (154 mg, 0.80 mmol, 1.60 equiv) in portions at room temperature under nitrogen atmosphere. The resulting mixture was stirred for 1 h at room temperature under nitrogen atmosphere. To the above mixture was added ethyl (3S)-3-amino-3-{4-fluoro-2′-hydroxy-5,6′-dimethyl-[1,1′-biphenyl]-3-yl}propanoate (167 mg, 0.50 mmol, 1.00 equiv) and NEt₃ (306 mg, 3.03 mmol, 6.00 equiv) in CH₃CN (6 mL) dropwise over 5 min at 0° C. The resulting mixture was stirred overnight at room temperature. The reaction was monitored by LCMS. The resulting mixture was concentrated under vacuum. The residue was purified by silica gel column chromatography, eluted with CH₂Cl₂/MeOH (0% to 5% gradient in 20 min) to afford ethyl (3S)-3-[2-(5-bromo-2-fluorophenyl)-2-{5-[2-(3-fluoroazetidin-1-yl)ethyl]-2-oxo-4-(trifluoromethyl)pyridin-1-yl}acetamido]-3-{4-fluoro-2′-hydroxy-5,6′-dimethyl-[1,1′-biphenyl]-3-yl}propanoate (260 mg, 63.7%) as a yellow solid. LC-MS: (ES+H, m/z): [M+H]⁺=808.1.

### Step 2: Preparation of ethyl 2-[(17S)-12,19-difluoro-14-{5-[2-(3-fluoroazetidin-1-yl)ethyl]-2-oxo-4-(trifluoromethyl)pyridin-1-yl}-3,20-dimethyl-15-oxo-8-oxa-16-azatetracyclo[16.3.1.1\hat{ }{9,13}.0\hat{ }{2,7}]tricosa-1(22),2,4,6,9,11,13(23),18,20-nonaen-17-yl]acetate

To a stirred solution of ethyl (3S)-3-[2-(5-bromo-2-fluorophenyl)-2-{5-[2-(3-fluoroazetidin-1-yl)ethyl]-2-oxo-4-(trifluoromethyl)pyridin-1-yl}acetamido]-3-{4-fluoro-2′-hydroxy-5,6′-dimethyl-[1,1′-biphenyl]-3-yl}propanoate (230 mg, 0.28 mmol, 1.00 equiv) in dioxane (15 mL) were added CuI (27 mg, 0.14 mmol, 0.50 equiv), K₂CO₃ (78 mg, 0.56 mmol, 2.00 equiv) and N1,N2-dimethylcyclohexane-1,2-diamine (40.46 mg, 0.28 mmol, 1.00 equiv) in portions at room temperature under nitrogen atmosphere. The resulting mixture was stirred overnight at 100° C. under nitrogen atmosphere and then cooled to room temperature. The resulting mixture was concentrated under reduced pressure. The residue was purified by silica gel column chromatography, eluted with CH₂Cl₂/MeOH (0% to 5% gradient in 20 min) to afford ethyl 2-[(17S)-12,19-difluoro-14-{5-[2-(3-fluoroazetidin-1-yl)ethyl]-2-oxo-4-(trifluoromethyl)pyridin-1-yl}-3,20-dimethyl-15-oxo-8-oxa-16-azatetracyclo[16.3.1.1\hat{ }{9,13}.0\hat{ }{2,7}]tricosa-1(22),2,4,6,9,11,13(23),18,20-nonaen-17-yl]acetate (150 mg, 72.5%) as a yellow solid. LC-MS: (ES+H, m/z): [M+H]⁺=728.3.

### Step 3: Preparation of [(14R*,17S)-12,19-difluoro-14-{5-[2-(3-fluoroazetidin-1-yl)ethyl]-2-oxo-4-(trifluoromethyl)pyridin-1-yl}-3,20-dimethyl-15-oxo-8-oxa-16-azatetracyclo[16.3.1.1\hat{ }{9,13}.0\hat{ }{2,7}]tricosa-1(21),2(7),3,5,9(23),10,12,18(22),19-nonaen-17-yl]acetic acid

To a stirred solution of ethyl 2-[(17S)-12,19-difluoro-14-{5-[2-(3-fluoroazetidin-1-yl)ethyl]-2-oxo-4-(trifluoromethyl)pyridin-1-yl}-3,20-dimethyl-15-oxo-8-oxa-16-azatetracyclo[16.3.1.1\hat{ }{9,13}.0\hat{ }{2,7}]tricosa-1(22),2,4,6,9,11,13(23),18,20-nonaen-17-yl]acetate (150 mg, 0.20 mmol, 1.00 equiv) in THF (2 mL) was added LiOH·H₂O (43 mg, 1.03 mmol, 5.00 equiv) in H₂O (0.5 mL) dropwise at room temperature under nitrogen atmosphere. The resulting mixture was stirred for 3 h at room temperature under nitrogen atmosphere. The reaction was monitored by LCMS. The residue was neutralized to pH 7 with HCl (2M, aq.). The resulting mixture was concentrated under reduced pressure. The residue was purified by reverse flash chromatography (Column: Xselect CSH C18 OBD Column 30*150 mm, 5 μm; Mobile Phase A: Water (0.1% HCOOH), Mobile Phase B: MeCN; Flow rate: 60 mL/min; Gradient: 24% B to 35% B in 8 min; Wavelength: 254 nm/220 nm; RT1 (min): 9.22) to afford [(14R*,17S)-12,19-difluoro-14-{5-[2-(3-fluoroazetidin-1-yl)ethyl]-2-oxo-4-(trifluoromethyl)pyridin-1-yl}-3,20-dimethyl-15-oxo-8-oxa-16-azatetracyclo[16.3.1.1\hat{ }{9,13}.0\hat{ }{2,7}]tricosa-1(21),2(7),3,5,9(23),10,12,18(22),19-nonaen-17-yl]acetic acid (73.2 mg, 50.7%). LC-MS: (ES+H, m/z): [M+H]⁺=700.20. ¹H NMR (300 MHz, CD₃OD) δ 7.63-7.51 (m, 1H), 7.46 (d, J=8.2 Hz, 1H), 7.42-7.30 (m, 2H), 7.24-7.12 (m, 2H), 6.99 (d, J=6.8 Hz, 1H), 6.89 (d, J=3.7 Hz, 2H), 6.49 (dd, J=6.4, 2.1 Hz, 1H), 6.12 (dd, J=6.1, 2.9 Hz, 1H), 5.72 (t, J=7.5 Hz, 1H), 5.15-4.87 (m, 1H), 3.62 (d, J=12.8 Hz, 2H), 3.25 (s, 2H), 2.86-2.63 (m, 4H), 2.55-2.40 CD₃OD (m, 2H), 2.36 (d, J=2.0 Hz, 3H), 2.02 (s, 3H). ¹⁹F NMR (282 MHz, CD₃OD) δ −65.00, −123.20, −126.75, −180.59.

### Example 8

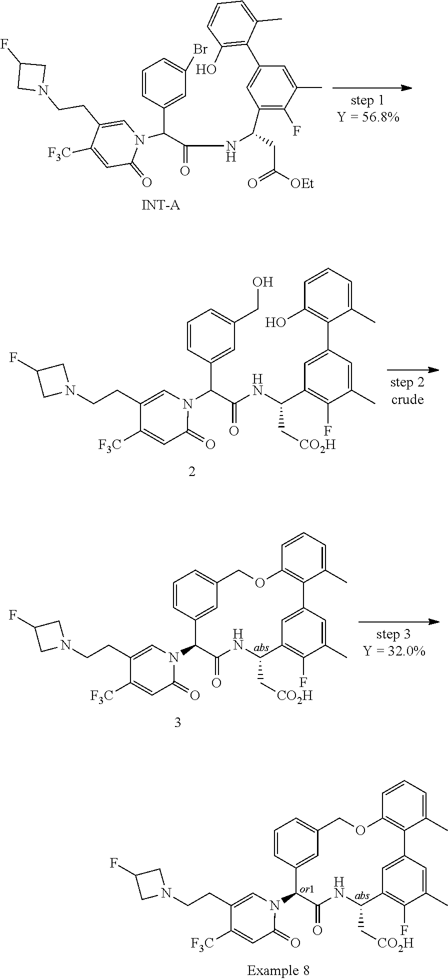

### Step 1: Preparation of rel-ethyl (3S)-3-{4-fluoro-2′-hydroxy-5,6′-dimethyl-[1,1′-biphenyl]-3-yl}-3-(2-{5-[2-(3-fluoroazetidin-1-yl)ethyl]-2-oxo-4-(trifluoromethyl)pyridin-1-yl}-2-[3-(hydroxymethyl)phenyl]acetamido)propanoate

To a stirred solution of rel-ethyl (3S)-3-[2-(3-bromophenyl)-2-{5-[2-(3-fluoroazetidin-1-yl)ethyl]-2-oxo-4-(trifluoromethyl)pyridin-1-yl}acetamido]-3-{4-fluoro-2′-hydroxy-5,6′-dimethyl-[1,1′-biphenyl]-3-yl}propanoate (150 mg, 0.19 mmol, 1.00 equiv) in dioxane (6 mL) were added 2nd Generation XPhos Precatalyst (15 mg, 0.02 mmol, 0.10 equiv) and (tributylstannyl)methanol (91 mg, 0.28 mmol, 1.50 equiv) dropwise at room temperature under nitrogen atmosphere. The resulting mixture was stirred overnight at 80° C. under nitrogen atmosphere and then cooled to room temperature. The resulting mixture was diluted with EtOAc (100 mL). The residue was washed with water (2×20 mL), dried over anhydrous Na₂SO₄, filtered and concentrated. The residue was purified by reversed-phase flash chromatography (C18; mobile phase, MeCN in water (0.1% NH₃·H₂O), 0% to 70% gradient in 40 min; detector, UV 220 nm) to provide rel-ethyl (3S)-3-{4-fluoro-2′-hydroxy-5,6′-dimethyl-[1,1′-biphenyl]-3-yl}-3-(2-{5-[2-(3-fluoroazetidin-1-yl)ethyl]-2-oxo-4-(trifluoromethyl)pyridin-1-yl}-2-[3-(hydroxymethyl)phenyl]acetamido)propanoate (80 mg, 56.8%) as a yellow solid. LC-MS: (ES+H, m/z): [M+H]⁺=742.3. ¹H NMR (300 MHz, CD₃OD) δ 7.39-7.20 (m, 5H), 6.98-6.86 (m, 3H), 6.69 (d, J=9.3 Hz, 2H), 6.64-6.57 (m, 2H), 5.62 (t, J=7.4 Hz, 1H), 4.95-4.80 (m, 1H), 4.54 (s, 2H), 3.97-3.89 (m, 2H), 3.40-3.28 (m, 2H), 2.96 (dddd, J=23.4, 9.8, 4.6, 1.7 Hz, 2H), 2.75 (d, J=7.4 Hz, 2H), 2.45-2.40 (m, 2H), 2.34 (q, J=7.1, 6.1 Hz, 2H), 2.18 (d, J=2.1 Hz, 3H), 1.82 (s, 3H), 1.02 (t, J=7.1 Hz, 3H).

### Step 2: Preparation of rel-ethyl 2-((9S)-14-fluoro-6-(5-(2-(3-fluoroazetidin-1-yl)ethyl)-2-oxo-4-(trifluoromethyl)pyridin-1(2H)-yl)-15,26-dimethyl-7-oxo-3-oxa-8-aza-1,5(1,3),2(1,2)-tribenzenacyclononaphane-9-yl)acetate

A solution of rel-ethyl (3S)-3-{4-fluoro-2′-hydroxy-5,6′-dimethyl-[1,1′-biphenyl]-3-yl}-3-(2-{5-[2-(3-fluoroazetidin-1-yl)ethyl]-2-oxo-4-(trifluoromethyl)pyridin-1-yl}-2-[3-(hydroxymethyl)phenyl]acetamido)propanoate (75 mg, 0.10 mmol, 1.00 equiv) and 2-(tributyl-lambda5-phosphanylidene)acetonitrile (244 mg, 1.01 mmol, 10.00 equiv) in PhCH₃ (8 mL) was stirred overnight at 100° C. under nitrogen atmosphere and then cooled to room temperature. The resulting mixture was diluted with EtOAc (100 mL). The residue was washed with water (2×20 mL). dried over anhydrous Na₂SO₄. After filtration, the filtrate was concentrated under reduced pressure. The residue was purified by silica gel column chromatography, eluted with CH₂Cl₂/MeOH (0% to 10% gradient in 20 min) to afford rel-ethyl 2-((9S)-14-fluoro-6-(5-(2-(3-fluoroazetidin-1-yl)ethyl)-2-oxo-4-(trifluoromethyl)pyridin-1(2H)-yl)-15,26-dimethyl-7-oxo-3-oxa-8-aza-1,5(1,3),2(1,2)-tribenzenacyclononaphane-9-yl)acetate (50 mg, crude) as a yellow solid. LC-MS: (ES+H, m/z): [M+H]⁺=724.2.

### Step 3: Preparation of rel-2-((6S,9S)-14-fluoro-6-(5-(2-(3-fluoroazetidin-1-yl)ethyl)-2-oxo-4-(trifluoromethyl)pyridin-1(2H)-yl)-15,26-dimethyl-7-oxo-3-oxa-8-aza-1,5(1,3),2(1,2)-tribenzenacyclononaphane-9-yl)acetic acid

To a stirred solution of rel-ethyl 2-((9S)-14-fluoro-6-(5-(2-(3-fluoroazetidin-1-yl)ethyl)-2-oxo-4-(trifluoromethyl)pyridin-1(2H)-yl)-15,26-dimethyl-7-oxo-3-oxa-8-aza-1,5(1,3),2(1,2)-tribenzenacyclononaphane-9-yl)acetate (50 mg, crude) in THF (1 mL) were added LiOH·H₂O (15 mg, 0.34 mmol, 5.00 equiv) and H₂O (0.2 mL) dropwise at room temperature under nitrogen atmosphere. The resulting mixture was stirred for 3 h at room temperature under nitrogen atmosphere. The reaction was monitored by LCMS. The mixture was neutralized to pH 7 with HCl (aq. 1M). The resulting mixture was concentrated under reduced pressure. The residue was purified by reversed flash chromatography (Column: XBridge Shield RP18 OBD Column, 50*250 mm, 10 μm; Mobile Phase A: Water (10 mmol/L HCO₂H), Mobile Phase B: MeCN; Flow rate: 100 mL/min; Gradient: 20% B to 60% B in 40 min, 40% B; Wavelength: 254/220 nm; RT1 (min): 20) to provide rel-2-((6S,9S)-14-fluoro-6-(5-(2-(3-fluoroazetidin-1-yl)ethyl)-2-oxo-4-(trifluoromethyl)pyridin-1(2H)-yl)-15,26-dimethyl-7-oxo-3-oxa-8-aza-1,5(1,3),2(1,2)-tribenzenacyclononaphane-9-yl)acetic acid (16.3 mg, 32.0%). LC-MS: (ES+H, m/z): [M+H]⁺=696.3. ¹H NMR (300 MHz, CD₃OD) δ 7.43-7.34 (m, 4H), 7.30-7.08 (m, 3H), 7.00-6.82 (m, 5H), 5.76 (t, J=7.0 Hz, 1H), 5.33 (d, J=13.2 Hz, 1H), 5.14-4.90 (m, 2H), 3.67-3.56 (m, 2H), 3.34-3.25 (m, 2H), 2.76-2.68 (m, 4H), 2.51 (d, J=7.5 Hz, 2H), 2.33 (s, 3H), 2.02 (s, 3H). ¹⁹F NMR (282 MHz, CD₃OD) δ −64.95, −128.30, −180.59.

### Example 9

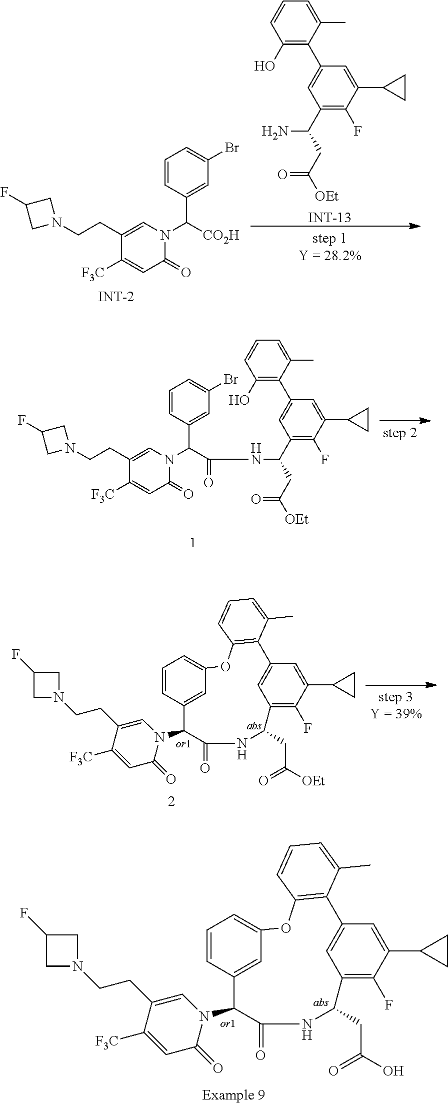

### Step 1: Preparation of ethyl (3S)-3-[2-(3-bromophenyl)-2-{5-[2-(3-fluoroazetidin-1-yl)ethyl]-2-oxo-4-(trifluoromethyl)pyridin-1-yl}acetamido]-3-{5-cyclopropyl-4-fluoro-2′-hydroxy-6′-methyl-[1,1′-biphenyl]-3-yl}propanoate

A solution of (3-bromophenyl)({5-[2-(3-fluoroazetidin-1-yl)ethyl]-2-oxo-4-(trifluoromethyl)pyridin-1-yl})acetic acid (589 mg, 1.23 mmol, 1.00 equiv) and HOBT (217 mg, 1.60 mmol, 1.30 equiv) and EDCI (355 mg, 1.85 mmol, 1.50 equiv) in CH₃CN (2.5 mL) was stirred for 1 h at room temperature under nitrogen atmosphere. To the above mixture was added ethyl (3S)-3-amino-3-{5-cyclopropyl-4-fluoro-2′-hydroxy-6′-methyl-[1,1′-biphenyl]-3-yl}propanoate (530 mg, 1.483 mmol, 1.2 equiv) and Et₃N (375.13 mg, 3.708 mmol, 3 equiv) in CH₃CN (1.5 mL) dropwise over 3 min at room temperature. The resulting mixture was stirred overnight at room temperature. The reaction was monitored by LCMS. The resulting mixture was diluted with EtOAc (100 mL). The residue was washed with water (3×50 mL), followed by brine (1×50 mL) and then concentrated under reduced pressure. The residue was purified by reversed-phase flash chromatography (C18; mobile phase, CH₃CN in water (0.1% NH₃·H₂O), 10% to 100% gradient in 40 min; detector, UV 254 nm) to provide ethyl (3S)-3-[2-(3-bromophenyl)-2-{5-[2-(3-fluoroazetidin-1-yl)ethyl]-2-oxo-4-(trifluoromethyl)pyridin-1-yl}acetamido]-3-{5-cyclopropyl-4-fluoro-2′-hydroxy-6′-methyl-[1,1′-biphenyl]-3-yl}propanoate (300 mg, 28.24%) as a yellow oil. LC-MS: (ES+H, m/z): [M+H]⁺=818.1.

### Step 2: Preparation of ethyl 2-[(17S)-20-cyclopropyl-19-fluoro-14-{5-[2-(3-fluoroazetidin-1-yl)ethyl]-2-oxo-4-(trifluoromethyl)pyridin-1-yl}-3-methyl-15-oxo-8-oxa-16-azatetracyclo[16.3.1.1\hat{ }{9,13}.0\hat{ }{2,7}]tricosa-1(22),2,4,6,9,11,13(23),18,20-nonaen-17-yl]acetate

To a stirred solution of ethyl (3S)-3-[2-(3-bromophenyl)-2-{5-[2-(3-fluoroazetidin-1-yl)ethyl]-2-oxo-4-(trifluoromethyl)pyridin-1-yl}acetamido]-3-{5-cyclopropyl-4-fluoro-2′-hydroxy-6′-methyl-[1,1′-biphenyl]-3-yl}propanoate (320 mg, 0.392 mmol, 1 equiv) and (1R,2R)—N1,N2-dimethylcyclohexane-1,2-diamine (55 mg, 0.39 mmol, 1.00 equiv) in dioxane (15 mL) were added CuI (37 mg, 0.19 mmol, 0.50 equiv) and K₂CO₃ (108 mg, 0.78 mmol, 2.00 equiv) in portions at room temperature. The resulting mixture was stirred overnight at 100° C. under nitrogen atmosphere and then cooled to room temperature. The resulting mixture was diluted with EtOAc (300 mL). The residue was washed with water (3×30 mL), followed by brine (1×30 mL) and then concentrated under reduced pressure. The residue was purified by silica gel column chromatography, eluted with CH₂Cl₂/MeOH (0%-10% gradient in 30 min) to afford ethyl 2-[(17S)-20-cyclopropyl-19-fluoro-14-{5-[2-(3-fluoroazetidin-1-yl)ethyl]-2-oxo-4-(trifluoromethyl)pyridin-1-yl}-3-methyl-15-oxo-8-oxa-16-azatetracyclo[16.3.1.1\hat{ }{9,13}.0\hat{ }{2,7}]tricosa-1(22),2,4,6,9,11,13(23),18,20-nonaen-17-yl]acetate (250 mg, 82.3%) as a yellow solid. LC-MS: (ES+H, m/z): [M+H]⁺=736.2. ¹H NMR (300 MHz, DMSO-d₆) δ8.93 (d, J=7.3 Hz, 1H), 7.58 (d, J=8.2 Hz, 1H), 7.55-7.49 (m, 1H), 7.40-7.36 (m, 2H), 7.22-7.10 (m, 3H), 6.80 (s, 1H), 6.65 (dd, J=7.0, 2.1 Hz, 1H), 6.47 (s, 1H), 6.36 (dd, J=6.5, 2.2 Hz, 1H), 6.01 (t, J=2.0 Hz, 1H), 5.55-5.48 (m, 1H), 4.89-4.71 (m, 1H), 4.12-3.96 (m, 2H), 3.29-3.16 (m, 2H), 2.92-2.70 (m, 4H), 2.46-2.24 (m, 5H), 2.23-2.01 (m, 2H), 2.00-1.92 (m, 1H), 1.12 (t, J=7.1 Hz, 3H), 1.00 (dd, J=8.4, 2.6 Hz, 2H), 0.82-0.66 (m, 2H).

### Step 3: Preparation of [(14R*,17S)-20-cyclopropyl-19-fluoro-14-{5-[2-(3-fluoroazetidin-1-yl)ethyl]-2-oxo-4-(trifluoromethyl)pyridin-1-yl}-3-methyl-15-oxo-8-oxa-16-azatetracyclo[16.3.1.1\hat{ }{9,13}.0\hat{ }{2,7}]tricosa-1(21),2(7),3,5,9,11,13(23),18(22),19-nonaen-17-yl]acetic acid

To a stirred solution of ethyl 2-[(17S)-20-cyclopropyl-19-fluoro-14-{5-[2-(3-fluoroazetidin-1-yl)ethyl]-2-oxo-4-(trifluoromethyl)pyridin-1-yl}-3-methyl-15-oxo-8-oxa-16-azatetracyclo[16.3.1.1\hat{ }{9,13}.0\hat{ }{2,7}]tricosa-1(22),2,4,6,9,11,13(23),18,20-nonaen-17-yl]acetate (200 mg, 0.27 mmol, 1.00 equiv) in THF (2 mL) and H₂O (0.4 mL) was added LiOH·H₂O (32.55 mg, 1.36 mmol, 5.00 equiv in portions at room temperature. The resulting mixture was stirred for 3 h at room temperature. The reaction was monitored by LCMS. The mixture was acidified to pH 7 with HCl (aq. 1 M). The resulting mixture was concentrated under vacuum. The crude product (200 mg) was purified by Prep-HPLC (Column: XBridge Shield RP18 OBD Column, 50*250 mm, 10 μm; Mobile Phase A: Water (0.05% HCO₂H), Mobile Phase B: CH₃CN; Flow rate: 25 mL/min; Gradient: 20% B to 50% B in 30 min, 39% B; Wavelength: 254/220 nm; RT1 (min): 19.2) to afford [(14R*,17S)-20-cyclopropyl-19-fluoro-14-{5-[2-(3-fluoroazetidin-1-yl)ethyl]-2-oxo-4-(trifluoromethyl)pyridin-1-yl}-3-methyl-15-oxo-8-oxa-16-azatetracyclo[16.3.1.1\hat{ }{9,13}.0\hat{ }{2,7}]tricosa-1(21),2(7),3,5,9,11,13(23),18(22),19-nonaen-17-yl]acetic acid (77.4 mg, 39.1%). LC-MS: (ES+H, m/z): [M+H]⁺=708.3. ¹H NMR (300 MHz, CD₃OD) δ7.44-7.33 (m, 5H), 7.15-7.09 (m, 2H), 6.87 (s, 1H), 6.66-6.60 (m, 2H), 6.34 (dd, J=6.3, 2.1 Hz, 1H), 6.19 (t, J=2.0 Hz, 1H), 5.71 (t, J=7.4 Hz, 1H), 5.16-4.91 (m, 1H), 3.68-3.65 (m, 2H), 3.35-3.27 (m, 2H), 2.75-2.67 (m, 4H), 2.50-2.48 (m, 2H), 2.17-2.16 (m, 1H), 1.96 (s, 3H), 1.03-0.99 (m, 2H), 0.78-0.67 (m, 2H). ¹⁹F NMR (377 MHz, CD₃OD) δ −64.97, −130.09, −180.43.

### Example 10

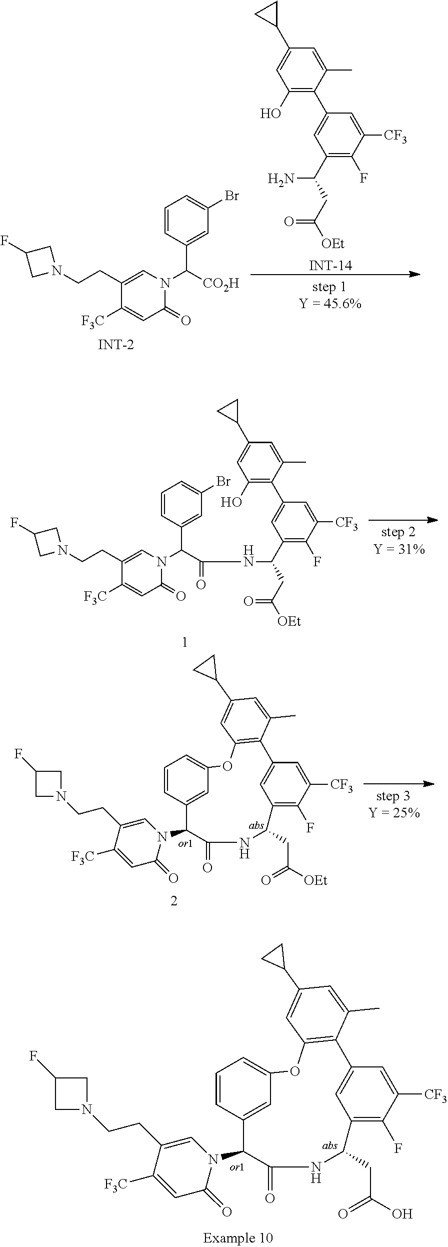

### Step 1: Preparation of ethyl (3S)-3-(2-(3-bromophenyl)-2-(5-(2-(3-fluoroazetidin-1-yl)ethyl)-2-oxo-4-(trifluoromethyl)pyridin-1(2H)-yl)acetamido)-3-(4′-cyclopropyl-4-fluoro-2′-hydroxy-6′-methyl-5-(trifluoromethyl)-[1,1′-biphenyl]-3-yl)propanoate

A solution of ethyl (S)-3-amino-3-(4′-cyclopropyl-4-fluoro-2′-hydroxy-6′-methyl-5-(trifluoromethyl)-[1,1′-biphenyl]-3-yl)propanoate hydrochloride (400 mg, assumed 100% yield, 0.87 mmol, 1.00 equiv) and triethylamine (526 mg, 5.20 mmol, 6.00 equiv) in MeCN (10 mL) was stirred for 30 min at 30° C. under nitrogen atmosphere. The resulting mixture was concentrated under reduced pressure. This resulted in ethyl (S)-3-amino-3-(4′-cyclopropyl-4-fluoro-2′-hydroxy-6′-methyl-5-(trifluoromethyl)-[1,1′-biphenyl]-3-yl)propanoate. To a stirred solution of 2-(3-bromophenyl)-2-(5-(2-(3-fluoroazetidin-1-yl)ethyl)-2-oxo-4-(trifluoromethyl)pyridin-1(2H)-yl)acetic acid (413 mg, 0.87 mmol, 1.00 equiv) in MeCN (10 mL) was added HOBT (152 mg, 1.13 mmol, 1.30 equiv) and EDCI (249 mg, 1.30 mmol, 1.50 equiv) at room temperature under nitrogen atmosphere. The resulting mixture was stirred for 1 h at 30° C. under nitrogen atmosphere. To the above mixture was added ethyl (S)-3-amino-3-(4′-cyclopropyl-4-fluoro-2′-hydroxy-6′-methyl-5-(trifluoromethyl)-[1,1′-biphenyl]-3-yl)propanoate in MeCN (0.5 mL) dropwise over 5 min at 0° C. The resulting mixture was stirred overnight at 30° C. The reaction was monitored by LCMS. Desired product could be detected by LCMS. The mixture was allowed to cool down to room temperature. The resulting mixture was concentrated under reduced pressure. The residue was purified by reversed-phase flash chromatography (C18; mobile phase, MeCN in water (0.11% NH₃·H₂O), 40% to 70% gradient in 30 min; detector, UV 220 nm) to provide ethyl (3S)-3-(2-(3-bromophenyl)-2-(5-(2-(3-fluoroazetidin-1-yl)ethyl)-2-oxo-4-(trifluoromethyl)pyridin-1(2H)-yl)acetamido)-3-(4′-cyclopropyl-4-fluoro-2′-hydroxy-6′-methyl-5-(trifluoromethyl)-[1,1′-biphenyl]-3-yl)propanoate (350 mg, 45.6%) as a light yellow solid. LC-MS: (ES+H, m/z): [M+H]⁺=884.2. ¹H NMR (300 MHz, CD₃OD) δ 7.70-7.60 (m, 1H), 7.51 (dd, J=6.5, 2.1 Hz, 1H), 7.48-7.32 (m, 4H), 7.24-7.15 (m, 1H), 6.91-6.69 (m, 2H), 6.54 (s, 1H), 6.47 (dd, J=4.4, 1.7 Hz, 1H), 5.76 (q, J=8.1, 7.6 Hz, 1H), 5.07 (q, J=5.2 Hz, 1H), 4.11 (qd, J=7.1, 2.8 Hz, 2H), 3.54-3.39 (m, 2H), 3.16-2.88 (m, 4H), 2.64-2.43 (m, 4H), 1.92-1.86 (m, 4H), 1.18 (td, J=7.1, 5.1 Hz, 3H), 1.01-0.92 (m, 2H), 0.72-0.64 (m, 2H). ¹⁹F NMR (282 MHz, CD₃OD) δ −62.41, −65.02, −125.05, −179.99.

### Step 2: Preparation of ethyl 2-((5S,8S)-2⁴-cyclopropyl-1⁴-fluoro-5-(5-(2-(3-fluoroazetidin-1-yl)ethyl)-2-oxo-4-(trifluoromethyl)pyridin-1(2H)-yl)-2⁶-methyl-6-oxo-1⁵-(trifluoromethyl)-3-oxa-7-aza-1,4(1,3),2(1,2)-tribenzenacyclooctaphane-8-yl)acetate

To a stirred mixture of ethyl (3S)-3-(2-(3-bromophenyl)-2-(5-(2-(3-fluoroazetidin-1-yl)ethyl)-2-oxo-4-(trifluoromethyl)pyridin-1(2H)-yl)acetamido)-3-(4′-cyclopropyl-4-fluoro-2′-hydroxy-6′-methyl-5-(trifluoromethyl)-[1,1′-biphenyl]-3-yl)propanoate (350 mg, 0.40 mmol, 1.00 equiv) and CuI (38 mg, 0.20 mmol, 0.50 equiv) in 1,4-dioxane (10 mL) was added (1R,2R)—N1,N2-dimethylcyclohexane-1,2-diamine (56 mg, 0.40 mmol, 1.00 equiv) and K₂CO₃ (109 mg, 0.79 mmol, 2.00 equiv) at room temperature. The resulting mixture was stirred overnight at 100° C. under nitrogen atmosphere and then cooled to room temperature. The resulting mixture was concentrated under reduced pressure. The residue was purified by Prep-HPLC (Column: Xselect CSH C18 OBD Column 30*150 mm, 5 μm; Mobile Phase A: Water (0.1% HCO₂H), Mobile Phase B: MeCN; Flow rate: 60 mL/min; Gradient: 35% B to 48% B in 8 min; Wavelength: 254 nm/220 nm; RT1 (min): 7.77) to afford ethyl 2-((5S,8S)-2⁴-cyclopropyl-1⁴-fluoro-5-(5-(2-(3-fluoroazetidin-1-yl)ethyl)-2-oxo-4-(trifluoromethyl)pyridin-1(2H)-yl)-2⁶-methyl-6-oxo-1⁵-(trifluoromethyl)-3-oxa-7-aza-1,4(1,3),2(1,2)-tribenzenacyclooctaphane-8-yl)acetate (100 mg, 31.4%) as a white solid. LC-MS: (ES+H, m/z): [M+H]⁺=804.2. ¹H NMR (300 MHz, CDCl₃) δ 7.60 (t, J=9.6 Hz, 1H), 7.44-7.31 (m, 4H), 7.17-7.05 (m, 2H), 6.91-6.86 (m, 1H), 6.83 (s, 1H), 6.75-6.65 (m, 2H), 6.35 (t, J=2.0 Hz, 1H), 5.76 (q, J=5.9 Hz, 1H), 5.05-4.73 (m, 1H), 4.12-3.88 (m, 2H), 3.06-2.85 (m, 2H), 2.81 (d, J=5.5 Hz, 2H), 2.62-2.33 (m, 4H), 2.01 (s, 3H), 1.92 (s, 3H), 1.15-1.05 (m, 5H), 0.85-0.76 (m, 2H). ¹⁹F NMR (377 MHz, CDCl₃) δ −61.14, −63.84, −122.27, −178.11.

### Step 3: Preparation of [(14R*,17S)-5-cyclopropyl-19-fluoro-14-{5-[2-(3-fluoroazetidin-1-yl)ethyl]-2-oxo-4-(trifluoromethyl)pyridin-1-yl}-3-methyl-15-oxo-20-(trifluoromethyl)-8-oxa-16-azatetracyclo[16.3.1.1\hat{ }{9,13}.0\hat{ }{2,7}]tricosa-1(21),2(7),3,5,9,11,13(23),18(22),19-nonaen-17-yl]acetic acid

A mixture of ethyl 2-((5S,8S)-2⁴-cyclopropyl-1⁴-fluoro-5-(5-(2-(3-fluoroazetidin-1-yl)ethyl)-2-oxo-4-(trifluoromethyl)pyridin-1(2H)-yl)-2⁶-methyl-6-oxo-1⁵-(trifluoromethyl)-3-oxa-7-aza-1,4(1,3),2(1,2)-tribenzenacyclooctaphane-8-yl)acetate (100 mg, 0.12 mmol, 1.00 equiv) and LiOH·H₂O (21 mg, 0.50 mmol, 4.00 equiv) in THF (2 mL) and H₂O (0.5 mL) was stirred for 2 h at room temperature under nitrogen atmosphere. The reaction was monitored by LCMS. Desired product could be detected by LCMS. The mixture was acidified to pH 5 with HCl (aq. 1 M). The resulting mixture was concentrated under reduced pressure. The residue was purified by Prep-HPLC (Column: Xbridge Phenyl OBD Column, 19*150 mm, 5 μm; Mobile Phase A: Water (10 mmol/L NH₄HCO₃+0.05% NH₃H₂O, Mobile Phase B: MeCN; Flow rate: 60 mL/min; Gradient: 34% B to 49% B in 8 min; Wavelength: 254 nm/220 nm; RT1 (min): 7.6) to afford [(14R*,17S)-5-cyclopropyl-19-fluoro-14-{5-[2-(3-fluoroazetidin-1-yl)ethyl]-2-oxo-4-(trifluoromethyl)pyridin-1-yl}-3-methyl-15-oxo-20-(trifluoromethyl)-8-oxa-16-azatetracyclo[16.3.1.1\hat{ }{9,13}.0\hat{ }{2,7}]tricosa-1(21),2(7),3,5,9,11,13(23),18(22),19-nonaen-17-yl]acetic acid (23.7 mg, 24.5%). LC-MS: (ES+H, m/z): [M+H]⁺=776.25. ¹H NMR (300 MHz, CD₃OD) δ 7.47 (dd, J=4.8, 2.0 Hz, 2H), 7.44-7.35 (m, 2H), 7.21 (d, J=1.8 Hz, 1H), 7.16 (dd, J=5.0, 2.5 Hz, 1H), 6.96-6.87 (m, 2H), 6.87-6.82 (m, 1H), 6.63 (s, 1H), 6.11 (d, J=2.2 Hz, 1H), 5.71 (t, J=7.3 Hz, 1H), 5.13-4.81 (m, 1H), 3.69-3.49 (m, 2H), 3.26 (dd, J=9.2, 4.5 Hz, 1H), 3.18 (dd, J=9.2, 4.5 Hz, 1H), 2.82-2.58 (m, 4H), 2.49 (q, J=7.4, 6.7 Hz, 2H), 2.09-1.98 (m, 1H), 1.96 (s, 3H), 1.16-0.99 (m, 2H), 0.93-0.74 (m, 2H). ¹⁹F NMR (282 MHz, CD₃OD) δ −62.49, −62.54, −64.98, 123.99, 124.03, 180.43.

### Example 11

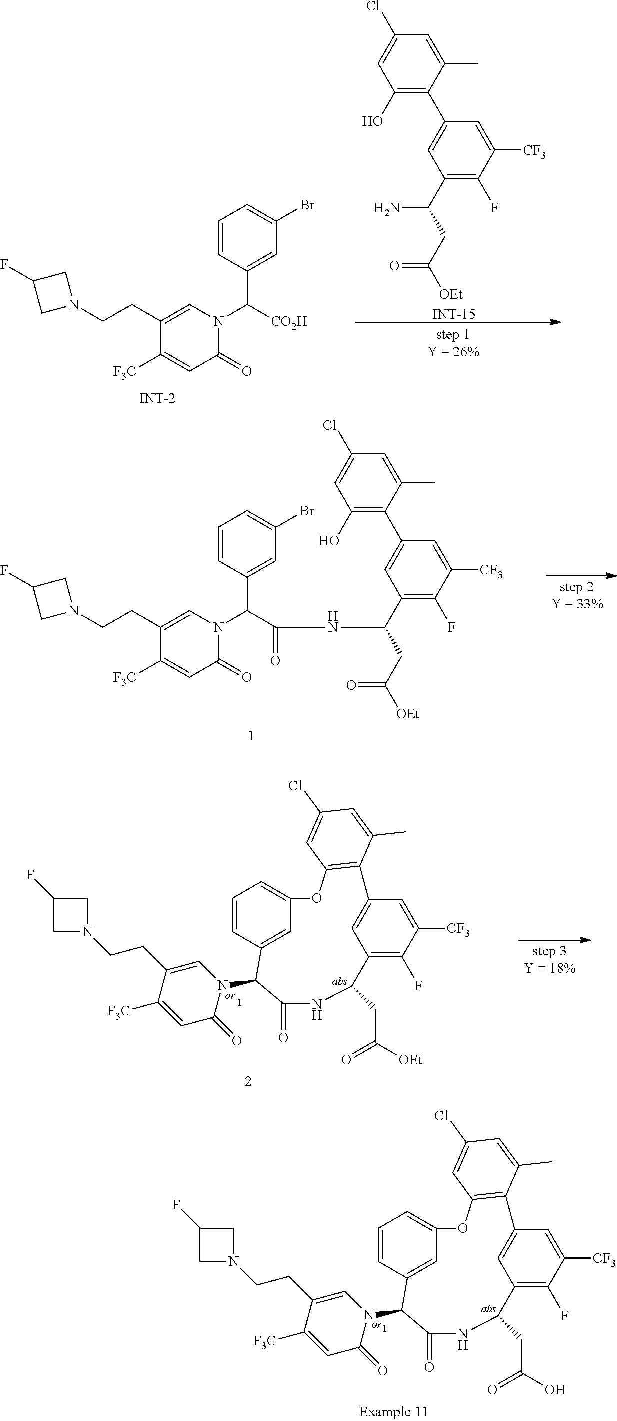

### Step 1: Preparation of ethyl (3S)-3-(2-(3-bromophenyl)-2-(5-(2-(3-fluoroazetidin-1-yl)ethyl)-2-oxo-4-(trifluoromethyl)pyridin-1(2H)-yl)acetamido)-3-(4′-chloro-4-fluoro-2′-hydroxy-6′-methyl-5-(trifluoromethyl)-[1,1′-biphenyl]-3-yl)propanoate

To a stirred mixture of (3-bromophenyl)({5-[2-(3-fluoroazetidin-1-yl)ethyl]-2-oxo-4-(trifluoromethyl)pyridin-1-yl})acetic acid (313 mg, 0.65 mmol, 1.00 equiv) in MeCN (6 mL) was added HOBT (124 mg, 0.92 mmol, 1.40 equiv) and EDCI (201.67 mg, 1.05 mmol, 1.60 equiv) in portions at room temperature under nitrogen atmosphere. The resulting mixture was stirred for 30 min at 30° C. under nitrogen atmosphere. To the above mixture was added ethyl (3S)-3-amino-3-[4′-chloro-4-fluoro-2′-hydroxy-6′-methyl-5-(trifluoromethyl)-[1,1′-biphenyl]-3-yl]propanoate hydrochloride (300 mg, assumed 100% yield, 0.65 mmol, 1.00 equiv) and Et₃N (133 mg, 1.32 mmol, 2.00 equiv) in MeCN (6 mL) dropwise over 20 min at 0° C. The resulting mixture was stirred overnight at 40° C. Desired product could be detected by LCMS. The resulting mixture was concentrated under reduced pressure. The residue was purified by silica gel column chromatography, eluted with CH₂Cl₂/MeOH (20:1 to 10:1 gradient in 30 min) to afford ethyl (3S)-3-(2-(3-bromophenyl)-2-(5-(2-(3-fluoroazetidin-1-yl)ethyl)-2-oxo-4-(trifluoromethyl)pyridin-1(2H)-yl)acetamido)-3-(4′-chloro-4-fluoro-2′-hydroxy-6′-methyl-5-(trifluoromethyl)-[1,1′-biphenyl]-3-yl)propanoate (150 mg, 25.9%) as a yellow solid. LC-MS: (ES+H, m/z): [M+H]⁺=878.0.

### Step 2: Preparation of ethyl 2-((8S)-24-chloro-1⁴-fluoro-5-(5-(2-(3-fluoroazetidin-1-yl)ethyl)-2-oxo-4-(trifluoromethyl)pyridin-1(2H)-yl)-2⁶-methyl-6-oxo-1⁵-(trifluoromethyl)-3-oxa-7-aza-1,4(1,3),2(1,2)-tribenzenacyclooctaphane-8-yl)acetate

A mixture of ethyl (3S)-3-[2-(3-bromophenyl)-2-{5-[2-(3-fluoroazetidin-1-yl)ethyl]-2-oxo-4-(trifluoromethyl)pyridin-1-yl}acetamido]-3-[4′-chloro-4-fluoro-2′-hydroxy-6′-methyl-5-(trifluoromethyl)-[1,1′-biphenyl]-3-yl]propanoate (150 mg, 0.17 mmol, 1.00 equiv), CuI (16 mg, 0.08 mmol, 0.50 equiv), (1R,2R)—N1,N2-dimethylcyclohexane-1,2-diamine (24 mg, 0.17 mmol, 1.00 equiv) and K₂CO₃ (47 mg, 0.34 mmol, 2.00 equiv) in 1,4-dioxane (5 mL) was stirred for 4 h at 100° C. under nitrogen atmosphere. Desired product could be detected by LCMS. The residue was purified by Prep-TLC (CH₂Cl₂/MeOH 20:1) to afford crude product. The crude product (150 mg) was purified by Prep-HPLC (Column: Xselect CSH C18 OBD Column 30*150 mm, 5 μm; Mobile Phase A: Water (0.1% HCOOH), Mobile Phase B: MeCN; Flow rate: 60 mL/min; Gradient: 34% B to 46% B in 8 min; Wavelength: 254 nm/220 nm) to afford ethyl 2-((8S)-24-chloro-14-fluoro-5-(5-(2-(3-fluoroazetidin-1-yl)ethyl)-2-oxo-4-(trifluoromethyl)pyridin-1(2H)-yl)-26-methyl-6-oxo-15-(trifluoromethyl)-3-oxa-7-aza-1,4(1,3),2(1,2)-tribenzenacyclooctaphane-8-yl)acetate (45 mg, 33.0%) as a yellow solid. LC-MS: (ES+H, m/z): [M+H]⁺=798.2. ¹H NMR (300 MHz, DMSO-d₆) δ 9.03 (d, J=6.7 Hz, 1H), 8.14 (s, 1H), 7.89 (d, J=2.1 Hz, 1H), 7.67-7.55 (m, 2H), 7.47 (t, J=8.0 Hz, 1H), 7.37 (d, J=2.0 Hz, 1H), 7.18 (d, J=9.8 Hz, 2H), 6.97-6.90 (m, 1H), 6.79 (s, 1H), 6.44 (s, 1H), 5.78 (t, J=2.0 Hz, 1H), 5.46 (q, J=7.5 Hz, 1H), 4.82 (dt, J=57.8, 5.2 Hz, 1H), 4.03 (qq, J=7.3, 3.7 Hz, 2H), 3.03-2.71 (m, 5H), 2.32 (d, J=11.7 Hz, 4H), 1.94 (s, 3H), 1.09 (t, J=7.1 Hz, 3H). ¹⁹F NMR (282 MHz, DMSO-d₆) δ −59.82, −62.63, −122.70, −177.04.

### Step 3: Preparation of 2-((5S,8S)-2⁴-chloro-1⁴-fluoro-5-(5-(2-(3-fluoroazetidin-1-yl)ethyl)-2-oxo-4-(trifluoromethyl)pyridin-1(2H)-yl)-2⁶-methyl-6-oxo-1⁵-(trifluoromethyl)-3-oxa-7-aza-1,4(1,3),2(1,2)-tribenzenacyclooctaphane-8-yl)acetic acid

A mixture of ethyl 2-[(17S)-5-chloro-19-fluoro-14-{5-[2-(3-fluoroazetidin-1-yl)ethyl]-2-oxo-4-(trifluoromethyl)pyridin-1-yl}-3-methyl-5-oxo-20-(trifluoromethyl)-8-oxa-1⁶-azatetracyclo[16.3.1.1\hat{ }{9,13}.0\hat{ }{2,7}]tricosa-1(22),2,4,6,9,11,13(23),18,20-nonaen-17-yl]acetate (45 mg, 0.056 mmol, 1 equiv) and LiOH (4 mg, 0.22 mmol, 4.00 equiv) in THF/water 1 mL (4/1) was stirred for 1 h at room temperature under nitrogen atmosphere. Desired product could be detected by LCMS. The mixture was acidified to pH 5 with HCl (aq. 1 M). The resulting mixture was concentrated under reduced pressure. The crude product (50 mg) was purified by Prep-HPLC (Column: Xselect CSH C18 OBD Column 30*150 mm, 5 μm; Mobile Phase A: Water (0.1% HCOOH), Mobile Phase B: MeCN; Flow rate: 60 mL/min; Gradient: 26% B to 43% B in 8 min; Wavelength: 254 nm/220 nm; RT1 (min): 10.03) to afford 2-((5S,8S)-2⁴-chloro-1⁴-fluoro-5-(5-(2-(3-fluoroazetidin-1-yl)ethyl)-2-oxo-4-(trifluoromethyl)pyridin-1(2H)-yl)-2⁶-methyl-6-oxo-15-(trifluoromethyl)-3-oxa-7-aza-1,4(1,3),2(1,2)-tribenzenacyclooctaphane-8-yl)acetic acid (8.1 mg, 17.9%). LC-MS: (ES+H, m/z): [M+H]⁺=770.2. ¹H NMR (400 MHz, DMSO-d₆) δ 9.15 (d, J=6.6 Hz, 1H), 7.88 (d, J=2.1 Hz, 1H), 7.62 (dd, J=8.2, 2.4 Hz, 1H), 7.55 (dd, J=6.4, 2.1 Hz, 1H), 7.46 (t, J=7.9 Hz, 1H), 7.36 (d, J=2.1 Hz, 1H), 7.16 (d, J=8.1 Hz, 2H), 6.92 (dd, J=6.3, 2.2 Hz, 1H), 6.78 (s, 1H), 6.46 (s, 1H), 5.80 (t, J=2.1 Hz, 1H), 5.43 (q, J=7.2 Hz, 1H), 4.82 (dp, J=57.6, 5.2 Hz, 1H), 3.23-3.14 (m, 2H), 2.89-2.68 (m, 4H), 2.42-2.23 (m, 4H), 1.94 (s, 3H). ¹⁹F NMR (377 MHz, DMSO-d₆) δ −59.70, −62.63, −122.64, −177.00.

### Example 12

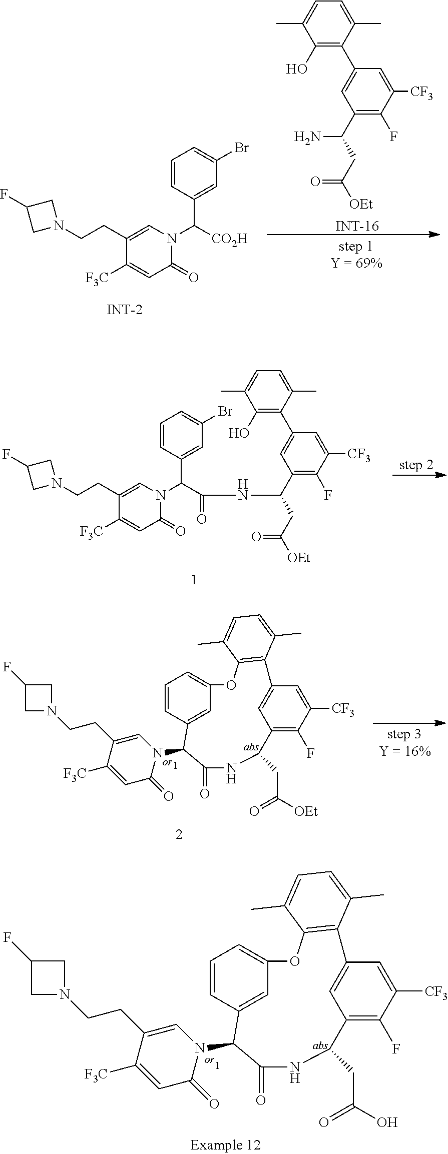

### Step 1: Preparation of ethyl (3S)-3-[2-(3-bromophenyl)-2-{5-[2-(3-fluoroazetidin-1-yl)ethyl]-2-oxo-4-(trifluoromethyl)pyridin-1-yl}acetamido]-3-[4-fluoro-2′-hydroxy-3′,6′-dimethyl-5-(trifluoromethyl)-[1,1′-biphenyl]-3-yl]propanoate

A solution of ethyl (3S)-3-amino-3-[4-fluoro-2′-hydroxy-3′,6′-dimethyl-5-(trifluoromethyl)-[1,1′-biphenyl]-3-yl]propanoate hydrochloride (368 mg, crude) and TEA (339 mg, 3.35 mmol, 4.00 equiv) in MeCN (5 mL) was stirred for 1 h at room temperature under nitrogen atmosphere. The resulting mixture was concentrated under reduced pressure. This resulted in ethyl (3S)-3-amino-3-[4-fluoro-2′-hydroxy-3′,6′-dimethyl-5-(trifluoromethyl)-[1,1′-biphenyl]-3-yl]propanoate. A solution of (3-bromophenyl)({5-[2-(3-fluoroazetidin-1-yl)ethyl]-2-oxo-4-(trifluoromethyl)pyridin-1-yl})acetic acid (400 mg, 0.83 mmol, 1.00 equiv), HOBT (169 mg, 1.25 mmol, 1.50 equiv) and EDCI (321 mg, 1.67 mmol, 2.00 equiv) in MeCN (5 mL) was stirred for 1 h at 40° C. under nitrogen atmosphere. To the above mixture was added ethyl (3S)-3-amino-3-[4-fluoro-2′-hydroxy-3′,6′-dimethyl-5-(trifluoromethyl)-[1,1′-biphenyl]-3-yl]propanoate in MeCN (3 mL) dropwise over 5 min at 0° C. The resulting mixture was stirred overnight at 40° C. The reaction was monitored by LCMS. Desired product could be detected by LCMS. The resulting mixture was concentrated under reduced pressure. The residue was purified by reversed-phase flash chromatography (C18 gel; mobile phase, MeCN in water (0.1% NH₃·H₂O), 30% to 70% gradient in 15 min; detector, UV 254 nm) to provide ethyl (3S)-3-[2-(3-bromophenyl)-2-{5-[2-(3-fluoroazetidin-1-yl)ethyl]-2-oxo-4-(trifluoromethyl)pyridin-1-yl}acetamido]-3-[4-fluoro-2′-hydroxy-3′,6′-dimethyl-5-(trifluoromethyl)-[1,1′-biphenyl]-3-yl]propanoate (500 mg, 69.4%) as light yellow solid. LC-MS: (ES+H, m/z): [M+H]⁺=858.35.

### Step 2: Preparation of ethyl 2-[(17S)-19-fluoro-14-{5-[2-(3-fluoroazetidin-1-yl)ethyl]-2-oxo-4-(trifluoromethyl)pyridin-1-yl}-3,6-dimethyl-15-oxo-20-(trifluoromethyl)-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate

To a stirred mixture of ethyl (3S)-3-[2-(3-bromophenyl)-2-{5-[2-(3-fluoroazetidin-1-yl)ethyl]-2-oxo-4-(trifluoromethyl)pyridin-1-yl}acetamido]-3-[4-fluoro-2′-hydroxy-3′,6′-dimethyl-5-(trifluoromethyl)-[1,1′-biphenyl]-3-yl]propanoate (160 mg, 0.186 mmol, 1.00 equiv) in DMSO (3 mL) was added CuCl (27 mg, 0.27 mmol, 1.50 equiv), Cs₂CO₃ (121 mg, 0.37 mmol, 2.00 equiv) and (1R,2R)—N1,N2-dimethylcyclohexane-1,2-diamine (26 mg, 0.18 mmol, 1.00 equiv) at room temperature under nitrogen atmosphere. The resulting mixture was stirred for an additional 2 h at 100° C. The reaction was monitored by LCMS. The mixture was allowed to cool down to room temperature. The resulting mixture was diluted with water (20 mL). The resulting mixture was extracted with EtOAc (4×20 mL). The combined organic layers were washed with brine (1×5 mL), dried over anhydrous Na₂SO₄. After filtration, the filtrate was concentrated under reduced pressure. The residue was purified by reversed-phase flash chromatography (C18; mobile phase, MeCN in water (10 mmol/L NH₄HCO₃), 20% to 70% gradient in 20 min; detector, UV 254 nm) to provide ethyl 2-[(17S)-19-fluoro-14-{5-[2-(3-fluoroazetidin-1-yl)ethyl]-2-oxo-4-(trifluoromethyl)pyridin-1-yl}-3,6-dimethyl-15-oxo-20-(trifluoromethyl)-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (50 mg, crude) as light yellow solid. LC-MS: (ES+H, m/z): [M+H]⁺=778.3.

### Step 3: Preparation of [(14R*,17S)-19-fluoro-14-{5-[2-(3-fluoroazetidin-1-yl)ethyl]-2-oxo-4-(trifluoromethyl)pyridin-1-yl}-3,6-dimethyl-15-oxo-20-(trifluoromethyl)-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid

To a stirred solution of ethyl 2-[(17S)-19-fluoro-14-{5-[2-(3-fluoroazetidin-1-yl)ethyl]-2-oxo-4-(trifluoromethyl)pyridin-1-yl}-3,6-dimethyl-15-oxo-20-(trifluoromethyl)-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (50 mg, crude) in THF (4 mL) and H₂O (0.8 mL) was added LiOH·H₂O (13 mg, 0.32 mmol, 5.00 equiv) at room temperature under nitrogen atmosphere. The resulting mixture was stirred for 2 h at room temperature. The reaction was monitored by LCMS. The mixture was acidified to pH 5 with conc. HCl (1 M). The resulting mixture was concentrated under reduced pressure. The crude product (50 mg) was purified by Prep-HPLC (Column: Xselect CSH C18 OBD Column 30*150 mm, 5 μm; Mobile Phase A: Water (0.1% HCOOH), Mobile Phase B: MeCN; Flow rate: 60 mL/min; Gradient: 23% B to 42% B in 8 min; Wavelength: 254 nm/220 nm; RT1 (min): 9.8) to afford [(14R*,17S)-19-fluoro-14-{5-[2-(3-fluoroazetidin-1-yl)ethyl]-2-oxo-4-(trifluoromethyl)pyridin-1-yl}-3,6-dimethyl-15-oxo-20-(trifluoromethyl)-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid (7.9 mg, 16.3%). LC-MS: (ES+H, m/z): [M+H]⁺=750.3. ¹H NMR (300 MHz, CD₃OD): δ 7.64 (s, 1H), 7.44 (t, J=7.9 Hz, 1H), 7.32 (d, J=7.8 Hz, 1H), 7.20 (t, J=8.6 Hz, 2H), 7.12 (d, J=7.8 Hz, 1H), 7.03-6.96 (m, 1H), 6.89 (s, 1H), 6.56 (d, J=5.7 Hz, 1H), 6.48 (s, 1H), 6.20 (s, 1H), 5.62 (s, 1H), 5.16-4.93 (m, 1H), 3.67 (dd, J=16.0, 8.6 Hz, 2H), 3.36 (d, J=10.9 Hz, 1H), 3.28-3.24 (m, 1H) 2.85-2.63 (m, 4H), 2.52 (s, 5H), 1.90 (s, 3H). ¹⁹F NMR (282 MHz, CD₃OD): δ −62.55, −64.95, −123.83, −180.79.

### Example 13

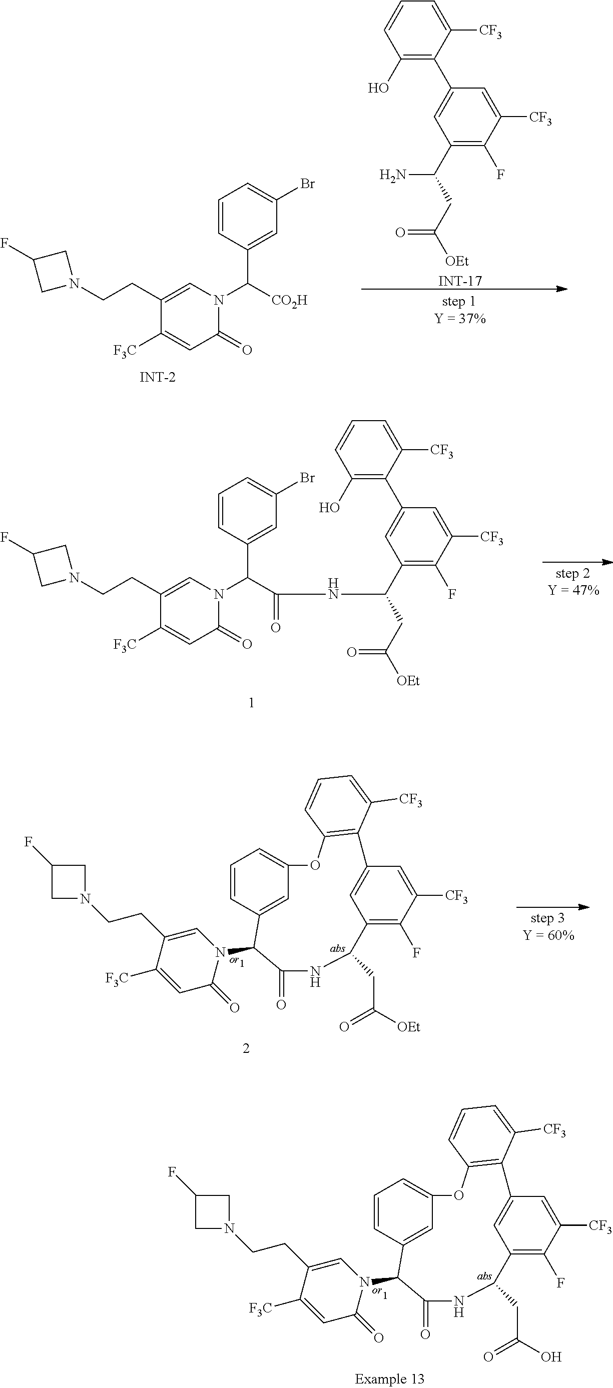

### Step 1: Preparation of ethyl (3S)-3-[2-(3-bromophenyl)-2-{5-[2-(3-fluoroazetidin-1-yl)ethyl]-2-oxo-4-(trifluoromethyl)pyridin-1-yl}acetamido]-3-[4-fluoro-2′-hydroxy-5,6′-bis(trifluoromethyl)-[1,1′-biphenyl]-3-yl]propanoate

To a stirred mixture of (3-bromophenyl)({5-[2-(3-fluoroazetidin-1-yl)ethyl]-2-oxo-4-(trifluoromethyl)pyridin-1-yl})acetic acid (300 mg, 0.63 mmol, 1.00 equiv) in CH₃CN (6 mL) were added HOBT (110 mg, 0.818 mmol, 1.30 equiv) and EDCI (180 mg, 0.94 mmol, 1.50 equiv) in portions at room temperature under nitrogen atmosphere. The resulting mixture was stirred for 1 h at room temperature under nitrogen atmosphere. To the above mixture was added ethyl (3S)-3-amino-3-[4-fluoro-2′-hydroxy-5,6′-bis(trifluoromethyl)-[1,1′-biphenyl]-3-yl]propanoate (276 mg, 0.62 mmol, 1.00 equiv) and NEt₃ (382 mg, 3.77 mmol, 6.00 equiv) in CH₃CN (6 mL) dropwise over 5 min at 0° C. The resulting mixture was stirred overnight at room temperature. The reaction was monitored by LCMS. The resulting mixture was concentrated under vacuum. The residue was purified by silica gel column chromatography, eluted with CH₂Cl₂/MeOH (0% to 5% gradient in 20 min) to ethyl (3S)-3-[2-(3-bromophenyl)-2-{5-[2-(3-fluoroazetidin-1-yl)ethyl]-2-oxo-4-(trifluoromethyl)pyridin-1-yl}acetamido]-3-[4-fluoro-2′-hydroxy-5,6′-bis(trifluoromethyl)-[1,1′-biphenyl]-3-yl]propanoate (210 mg, 37.2%) as a yellow solid. LC-MS: (ES+H, m/z): [M+H]⁺=898.0.

### Step 2: Preparation of ethyl 2-[(17S)-19-fluoro-14-{5-[2-(3-fluoroazetidin-1-yl)ethyl]-2-oxo-4-(trifluoromethyl)pyridin-1-yl}-15-oxo-3,20-bis(trifluoromethyl)-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (85 mg, 46.71%)

To a stirred solution of ethyl (3S)-3-[2-(3-bromophenyl)-2-{5-[2-(3-fluoroazetidin-1-yl)ethyl]-2-oxo-4-(trifluoromethyl)pyridin-1-yl}acetamido]-3-[4-fluoro-2′-hydroxy-5,6′-bis(trifluoromethyl)-[1,1′-biphenyl]-3-yl]propanoate (200 mg, 0.22 mmol, 1.00 equiv) in dioxane (15 mL) were added CuI (21 mg, 0.11 mmol, 0.50 equiv), K₂CO₃ (62 mg, 0.44 mmol, 2.00 equiv) and N1,N2-dimethylcyclohexane-1,2-diamine (32 mg, 0.22 mmol, 1.00 equiv) in portions at room temperature under nitrogen atmosphere. The resulting mixture was stirred overnight at 100° C. under nitrogen atmosphere and then cooled to room temperature. The resulting mixture was concentrated under reduced pressure. The residue was purified by silica gel column chromatography, eluted with CH₂Cl₂/MeOH (0% to 5% gradient in 20 min) to afford ethyl 2-[(17S)-19-fluoro-14-{5-[2-(3-fluoroazetidin-1-yl)ethyl]-2-oxo-4-(trifluoromethyl)pyridin-1-yl}-15-oxo-3,20-bis(trifluoromethyl)-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (85 mg, 46.7%) as a yellow solid. LC-MS: (ES+H, m/z): [M+H]⁺=818.2.

### Step 3: Preparation of [(14R*,17S)-12,19-difluoro-14-{5-[2-(3-fluoroazetidin-1-yl)ethyl]-2-oxo-4-(trifluoromethyl)pyridin-1-yl}-3,20-dimethyl-15-oxo-8-oxa-16-azatetracyclo[16.3.1.1\hat{ }{9,13}.0\hat{ }{2,7}]tricosa-1(21),2(7),3,5,9(23),10,12,18(22),19-nonaen-17-yl]acetic acid

To a stirred solution of ethyl 2-[(17S)-19-fluoro-14-{5-[2-(3-fluoroazetidin-1-yl)ethyl]-2-oxo-4-(trifluoromethyl)pyridin-1-yl}-15-oxo-3,20-bis(trifluoromethyl)-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (80 mg, 0.09 mmol, 1.00 equiv) in THF (1.5 mL) was added LiOH·H₂O (12 mg, 0.49 mmol, 5.00 equiv) in H₂O (0.5 mL) dropwise at room temperature under nitrogen atmosphere. The resulting mixture was stirred for 3 h at room temperature under nitrogen atmosphere. The reaction was monitored by LCMS. The residue was neutralized to pH 7 with HCl (2 M, aq.). The resulting mixture was concentrated under reduced pressure. The residue was purified by reverse flash chromatography (Column: XSelect CSH Prep C18 OBD Column, 30*150 mm, 5 μm; Mobile Phase A: Water (0.1% HCOOH), Mobile Phase B: MeCN; Flow rate: 60 mL/min; Gradient: 23% B to 37% B in 10 min; Wavelength: 254 nm/220 nm; RT1 (min): 10.02) to afford [(14R*,17S)-19-fluoro-14-{5-[2-(3-fluoroazetidin-1-yl)ethyl]-2-oxo-4-(trifluoromethyl)pyridin-1-yl}-15-oxo-3,20-bis(trifluoromethyl)-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid (46.5 mg, 60.2%). LC-MS: (ES+H, m/z): [M+H]⁺=790.20. ¹H NMR (300 MHz, CD₃OD) δ 8.03 (dd, J=6.4, 3.0 Hz, 1H), 7.76 (q, J=5.0, 4.3 Hz, 2H), 7.58-7.43 (m, 3H), 7.35 (s, 1H), 7.19 (d, J=7.2 Hz, 1H), 6.98 (d, J=5.3 Hz, 1H), 6.90 (s, 1H), 6.64 (s, 1H), 6.02 (s, 1H), 5.71 (t, J=7.5 Hz, 1H), 5.14-5.05 (m, 1H), 3.62 (s, 2H), 3.24 (s, 2H), 2.91-2.64 (m, 4H), 2.58-2.40 (m, 2H). ¹⁹F NMR (282 MHz, CD₃OD) δ −59.12, −62.62, −64.98, −122.08, −180.54.

### Example 14

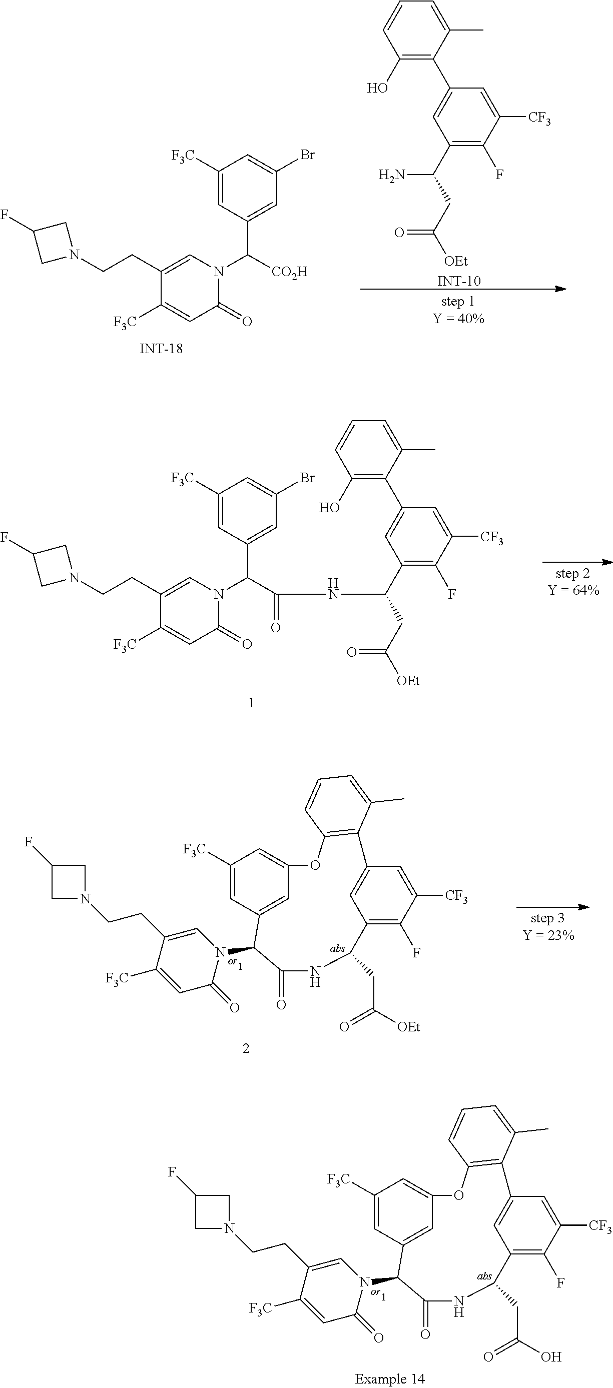

### Step 1: Preparation of ethyl (3S)-3-{2-[3-bromo-5-(trifluoromethyl)phenyl]-2-{5-[2-(3-fluoroazetidin-1-yl)ethyl]-2-oxo-4-(trifluoromethyl)pyridin-1-yl}acetamido}-3-[4-fluoro-2′-hydroxy-6′-methyl-5-(trifluoromethyl)-[1,1′-biphenyl]-3-yl]propanoate

To a stirred mixture of [3-bromo-5-(trifluoromethyl)phenyl]({5-[2-(3-fluoroazetidin-1-yl)ethyl]-2-oxo-4-(trifluoromethyl)pyridin-1-yl})acetic acid (240 mg, 0.44 mmol, 1.00 equiv) and HOBT (77 mg, 0.57 mmol, 1.30 equiv) in MeCN (12 mL) was added EDCI (126 mg, 0.66 mmol, 1.50 equiv) at room temperature under nitrogen atmosphere. The resulting mixture was stirred for 1 h at 40° C. under nitrogen atmosphere. To the above mixture was added ethyl (3S)-3-amino-3-[4-fluoro-2′-hydroxy-6′-methyl-5-(trifluoromethyl)-[1,1′-biphenyl]-3-yl]propanoate (169 mg, 0.44 mmol, 1.00 equiv) and NEt₃ (267 mg, 2.64 mmol, 6.00 equiv) at room temperature. The resulting mixture was stirred for an additional 3 days at 40° C. The reaction was monitored by LCMS. The residue was purified by reversed-phase flash chromatography (C18; mobile phase, MeCN in water (0.1% NH₃·H₂O), 30% to 80% gradient in 30 min; detector, UV 220 nm) to provide ethyl (3S)-3-{2-[3-bromo-5-(trifluoromethyl)phenyl]-2-{5-[2-(3-fluoroazetidin-1-yl)ethyl]-2-oxo-4-(trifluoromethyl)pyridin-1-yl}acetamido}-3-[4-fluoro-2′-hydroxy-6′-methyl-5-(trifluoromethyl)-[1,1′-biphenyl]-3-yl]propanoate (160 mg, 39.8%) as a white solid. LC-MS: (ES+H, m/z): [M+H]⁺=912.0.

### Step 2: Preparation of ethyl 2-[(17S)-19-fluoro-14-{5-[2-(3-fluoroazetidin-1-yl)ethyl]-2-oxo-4-(trifluoromethyl)pyridin-1-yl}-3-methyl-15-oxo-11,20-bis(trifluoromethyl)-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate

To a stirred mixture of ethyl (3S)-3-{2-[3-bromo-5-(trifluoromethyl)phenyl]-2-{5-[2-(3-fluoroazetidin-1-yl)ethyl]-2-oxo-4-(trifluoromethyl)pyridin-1-yl}acetamido}-3-[4-fluoro-2′-hydroxy-6′-methyl-5-(trifluoromethyl)-[1,1′-biphenyl]-3-yl]propanoate (145 mg, 0.15 mmol, 1.00 equiv) and (1R,2R)—N1,N2-dimethylcyclohexane-1,2-diamine (27.12 mg, 0.19 mmol, 1.20 equiv) in dioxane (10 mL) were added CuI (9 mg, 0.04 mmol, 0.30 equiv) and K₂CO₃ (43 mg, 0.31 mmol, 2.00 equiv) at room temperature under nitrogen atmosphere. The resulting mixture was stirred for 2 h at 100° C. under nitrogen atmosphere. The reaction was monitored by LCMS. The resulting mixture was filtered and the filter cake was washed with EtOAc (3×100 mL). The filtrate was concentrated under reduced pressure. The residue was purified by reversed combi-flash chromatography (C18; mobile phase, MeCN in water (0.1% NH₃·H₂O), 20% to 70% gradient in 30 min; detector, UV 220 nm) to provide ethyl 2-[(17S)-19-fluoro-14-{5-[2-(3-fluoroazetidin-1-yl)ethyl]-2-oxo-4-(trifluoromethyl)pyridin-1-yl}-3-methyl-15-oxo-11,20-bis(trifluoromethyl)-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (85 mg, 64.3%) as a white solid. LC-MS: (ES+H, m/z): [M+H]⁺=832.1.

### Step 3: Preparation of [(14R*,17S)-19-fluoro-14-{5-[2-(3-fluoroazetidin-1-yl)ethyl]-2-oxo-4-(trifluoromethyl)pyridin-1-yl}-3-methyl-15-oxo-11,20-bis(trifluoromethyl)-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid

To a stirred mixture of ethyl 2-[(17S)-19-fluoro-14-{5-[2-(3-fluoroazetidin-1-yl)ethyl]-2-oxo-4-(trifluoromethyl)pyridin-1-yl}-3-methyl-15-oxo-11,20-bis(trifluoromethyl)-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (80 mg, 0.09 mmol, 1.00 equiv) and H₂O (1 mL) in THF (3 mL) was added LiOH·H₂O (5 mg, 0.19 mmol, 2.00 equiv) at room temperature under nitrogen atmosphere. The resulting mixture was stirred for 1 h at room temperature under nitrogen atmosphere. The reaction was monitored by LCMS. The mixture was acidified to pH 6 with 1 N HCl (aq.). The resulting mixture was concentrated under reduced pressure. The crude product (100 mg) was purified by Prep-HPLC (Column: Xselect Prep OBD C18 Column, 30*150 mm, 5 μm; Mobile Phase A: Water (0.1% HCOOH), Mobile Phase B: MeCN; Flow rate: 60 mL/min; Gradient: 27% B to 44% B in 8 min; Wavelength: 254 nm/220 nm; RT1 (min): 9.5) to afford [(14R*,17S)-19-fluoro-14-{5-[2-(3-fluoroazetidin-1-yl)ethyl]-2-oxo-4-(trifluoromethyl)pyridin-1-yl}-3-methyl-15-oxo-11,20-bis(trifluoromethyl)-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid (17.8 mg, 23.0%). LC-MS: (ES+H, m/z): [M+H]⁺=804.1. ¹H NMR (300 MHz, DMSO-d₆) δ 9.10 (d, J=6.7 Hz, 1H), 7.86 (t, J=4.3 Hz, 2H), 7.68 (s, 1H), 7.56-7.52 (m, 1H), 7.48 (t, J=7.9 Hz, 1H), 7.27 (d, J=7.5 Hz, 1H), 7.19 (s, 1H), 6.94-6.84 (m, 1H), 6.80 (s, 1H), 6.50 (s, 1H), 6.08 (t, J=1.9 Hz, 1H), 5.43 (q, J=7.3 Hz, 1H), 4.80 (dt, J=57.8, 5.3 Hz, 1H), 3.26-3.19 (m, 2H), 2.91-2.70 (m, 4H), 2.34 (d, J=9.5 Hz, 4H), 1.95 (s, 3H). ¹⁹F NMR (282 MHz, DMSO-d₆) δ −59.71, −60.94, −62.62, −122.92, −177.24.

### Example 15

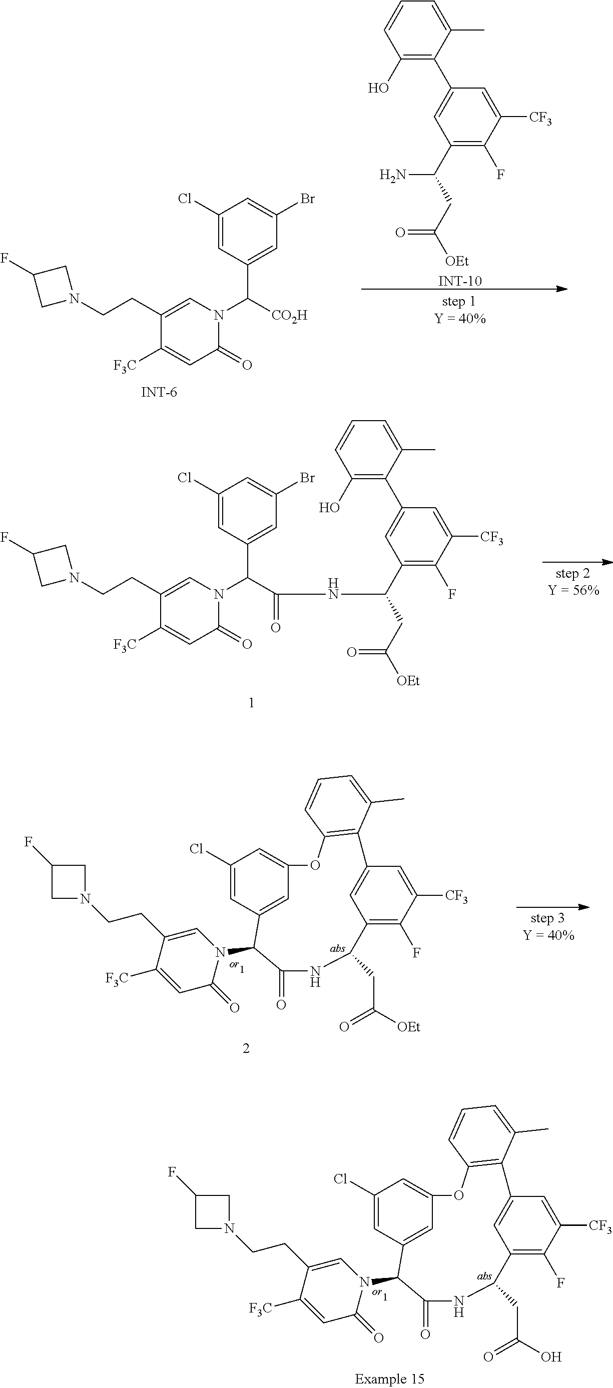

### Step 1: Preparation of ethyl (3S)-3-[2-(3-bromo-5-chlorophenyl)-2-{5-[2-(3-fluoroazetidin-1-yl)ethyl]-2-oxo-4-(trifluoromethyl)pyridin-1-yl}acetamido]-3-[4-fluoro-2′-hydroxy-6′-methyl-5-(trifluoromethyl)-[1,1′-biphenyl]-3-yl]propanoate

To a stirred mixture of (3-bromo-5-chlorophenyl)({5-[2-(3-fluoroazetidin-1-yl)ethyl]-2-oxo-4-(trifluoromethyl)pyridin-1-yl})acetic acid (250 mg, 0.48 mmol, 1.00 equiv) and HOBT (85 mg, 0.63 mmol, 1.30 equiv) in MeCN (9 mL) was added EDCI (140 mg, 0.73 mmol, 1.50 equiv) at room temperature under nitrogen atmosphere. The resulting mixture was stirred for 1 h at 40° C. under nitrogen atmosphere. To the above mixture was added ethyl (3S)-3-amino-3-[4-fluoro-2′-hydroxy-6′-methyl-5-(trifluoromethyl)-[1,1′-biphenyl]-3-yl]propanoate (188 mg, 0.48 mmol, 1.00 equiv) and NEt₃ (296 mg, 2.93 mmol, 6.00 equiv) at room temperature. The resulting mixture was stirred for an additional 3 days at 40° C. The reaction was monitored by LCMS. The residue was purified by reversed combi-flash chromatography (C18 gel; mobile phase, MeCN in water (0.1% NH₃·H₂O), 30% to 80% gradient in 30 min; detector, UV 220 nm) to afford ethyl (3S)-3-[2-(3-bromo-5-chlorophenyl)-2-{5-[2-(3-fluoroazetidin-1-yl)ethyl]-2-oxo-4-(trifluoromethyl)pyridin-1-yl}acetamido]-3-[4-fluoro-2′-hydroxy-6′-methyl-5-(trifluoromethyl)-[1,1′-biphenyl]-3-yl]propanoate (170 mg, 39.5%) as a white solid. LC-MS: (ES+H, m/z): [M+H]⁺=878.0.

### Step 2: Preparation of ethyl 2-[(17S)-11-chloro-19-fluoro-14-{5-[2-(3-fluoroazetidin-1-yl)ethyl]-2-oxo-4-(trifluoromethyl)pyridin-1-yl}-3-methyl-15-oxo-20-(trifluoromethyl)-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate

To a stirred mixture of ethyl (3S)-3-[2-(3-bromo-5-chlorophenyl)-2-{5-[2-(3-fluoroazetidin-1-yl)ethyl]-2-oxo-4-(trifluoromethyl)pyridin-1-yl}acetamido]-3-[4-fluoro-2′-hydroxy-6′-methyl-5-(trifluoromethyl)-[1,1′-biphenyl]-3-yl]propanoate (167 mg, 0.19 mmol, 1.00 equiv) and (1R,2R)—N1,N2-dimethylcyclohexane-1,2-diamine (33 mg, 0.22 mmol, 1.20 equiv) in dioxane (10 mL) were added CuI (11 mg, 0.05 mmol, 0.30 equiv) and K₂CO₃ (53 mg, 0.38 mmol, 2.00 equiv) at room temperature under nitrogen atmosphere. The resulting mixture was stirred for 4 h at 100° C. under nitrogen atmosphere. The reaction was monitored by LCMS. The resulting mixture was filtered and the filter cake was washed with EtOAc (3×100 mL). The filtrate was concentrated under reduced pressure. The residue was purified by reversed combi-flash chromatography (C18; mobile phase, MeCN in water (0.1% NH₃·H₂O), 20% to 70% gradient in 30 min; detector, UV 220 nm) to provide ethyl 2-[(17S)-11-chloro-19-fluoro-14-{5-[2-(3-fluoroazetidin-1-yl)ethyl]-2-oxo-4-(trifluoromethyl)pyridin-1-yl}-3-methyl-15-oxo-20-(trifluoromethyl)-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (85 mg, 56.0%) as a white solid. LC-MS: (ES+H, m/z): [M+H]⁺=798.1.

### Step 3: Preparation of [(14R*,17S)-11-chloro-19-fluoro-14-{5-[2-(3-fluoroazetidin-1-yl)ethyl]-2-oxo-4-(trifluoromethyl)pyridin-1-yl}-3-methyl-15-oxo-20-(trifluoromethyl)-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid

To a stirred mixture of ethyl 2-[(17S)-11-chloro-19-fluoro-14-{5-[2-(3-fluoroazetidin-1-yl)ethyl]-2-oxo-4-(trifluoromethyl)pyridin-1-yl}-3-methyl-15-oxo-20-(trifluoromethyl)-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (80 mg, 0.10 mmol, 1.00 equiv) and H₂O (1 mL) in THF (3 mL) was added LiOH·H₂O (9 mg, 0.20 mmol, 2.00 equiv) at room temperature under nitrogen atmosphere. The resulting mixture was stirred for 1 h at room temperature under nitrogen atmosphere. The reaction was monitored by LCMS. The mixture was acidified to pH 6 with 1 N HCl (aq.). The resulting mixture was concentrated under reduced pressure. The crude product (100 mg) was purified by Prep-HPLC (Column: XBridge Prep OBD C18 Column, 30*150 mm, 5 μm; Mobile Phase A: Water (0.1% HCO₂H), Mobile Phase B: MeCN; Flow rate: 60 mL/min; Gradient: 27% B to 44% B in 8 min; Wavelength: 254 nm/220 nm; RT1 (min): 9.5) to provide [(14R*,17S)-11-chloro-19-fluoro-14-{5-[2-(3-fluoroazetidin-1-yl)ethyl]-2-oxo-4-(trifluoromethyl)pyridin-1-yl}-3-methyl-15-oxo-20-(trifluoromethyl)-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid (30.9 mg, 40.0%). LC-MS: (ES+H, m/z): [M+H]⁺=770.20/772.20. ¹H NMR (400 MHz, DMSO-d₆) δ 12.44 (s, 1H), 9.02 (d, J=6.8 Hz, 1H), 7.74 (d, J=8.1 Hz, 1H), 7.62 (t, J=2.0 Hz, 1H), 7.52 (dd, J=6.3, 2.1 Hz, 1H), 7.46 (t, J=7.9 Hz, 1H), 7.35 (d, J=1.7 Hz, 1H), 7.27 (d, J=7.7 Hz, 1H), 7.20 (s, 1H), 6.91 (dd, J=6.2, 2.2 Hz, 1H), 6.83 (s, 1H), 6.41 (s, 1H), 5.81 (s, 1H), 5.44 (q, J=7.3 Hz, 1H), 4.96 (d, J=56.7 Hz, 1H), 3.21-3.18 (m, 2H), 2.90-2.53 (m, 4H), 2.40 (s, 4H), 1.95 (s, 3H). ¹⁹F NMR (282 MHz, DMSO-d₆) δ −59.67, −62.53, −122.65, −177.18.

### Example 16

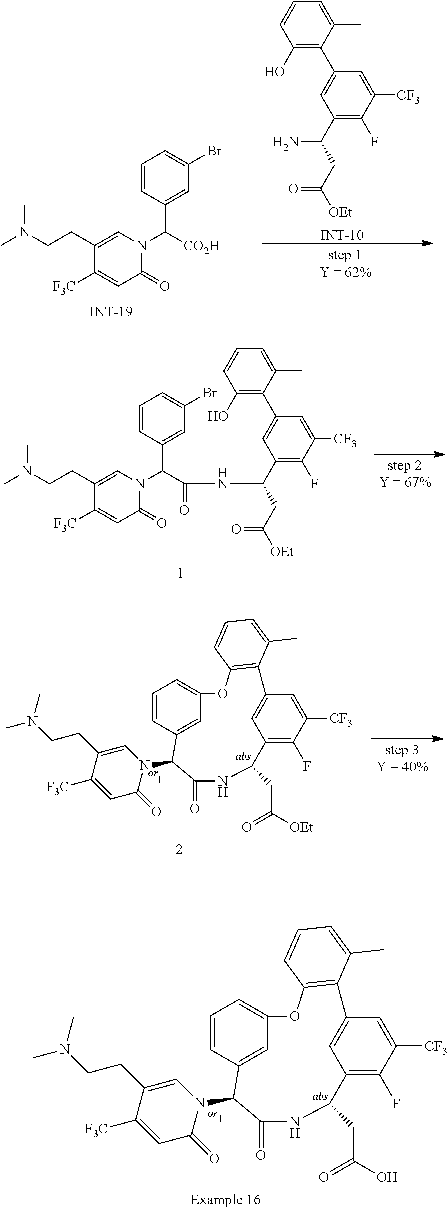

### Step 1: Preparation of ethyl (3S)-3-[2-(3-bromophenyl)-2-{5-[2-(dimethylamino)ethyl]-2-oxo-4-(trifluoromethyl)pyridin-1-yl}acetamido]-3-[4-fluoro-2′-hydroxy-6′-methyl-5-(trifluoromethyl)-[1,1′-biphenyl]-3-yl]propanoate

To a stirred mixture of (3-bromophenyl)({5-[2-(dimethylamino)ethyl]-2-oxo-4-(trifluoromethyl)pyridin-1-yl})acetic acid (300 mg, 0.67 mmol, 1.00 equiv) in MeCN (3 mL) were added EDCI (376 mg, 1.34 mmol, 2.00 equiv) and HOBT (135 mg, 1.00 mmol, 1.50 equiv) at room temperature under nitrogen atmosphere. To the above mixture was added ethyl (3S)-3-amino-3-[4-fluoro-2′-hydroxy-6′-methyl-5-(trifluoromethyl)-[1,1′-biphenyl]-3-yl]propanoate (310 mg, 0.80 mmol, 1.20 equiv) and Et₃N (407 mg, 4.02 mmol, 6.00 equiv) in MeCN (3 mL) dropwise over 1 min at room temperature. The resulting mixture was stirred for additional 2 h at room temperature and then was concentrated. The residue was purified by reversed-phase flash chromatography (C18; mobile phase, MeCN in water (0.1% NH₃·H₂O), 10% to 50% gradient in 10 min; detector, UV 254 nm/220 nm) to afford ethyl (3S)-3-[2-(3-bromophenyl)-2-{5-[2-(dimethylamino)ethyl]-2-oxo-4-(trifluoromethyl)pyridin-1-yl}acetamido]-3-[4-fluoro-2′-hydroxy-6′-methyl-5-(trifluoromethyl)-[1,1′-biphenyl]-3-yl]propanoate (340 mg, 62.2%) as yellow oil. LC-MS: (ES+H, m/z): [M+H]⁺=814.1. ¹H NMR (400 MHz, DMSO-d₆) δ 9.38 (s, 1H), 7.69-7.57 (m, 1H), 7.55-7.51 (m, 1H), 7.50-7.40 (m, 3H), 7.36-7.31 (m, 1H), 7.23-7.05 (m, 2H), 6.82-6.70 (m, 3H), 6.61 (s, 1H), 5.72-5.57 (m, 1H), 4.11 (q, J=5.3 Hz, 4H), 4.05-3.97 (m, 2H), 3.17 (d, J=5.2 Hz, 6H), 2.94-2.89 (m, 1H), 2.48-2.40 (m, 2H), 2.33-2.09 (m, 3H), 1.08 (t, J=7.1, 1.1 Hz, 3H). ¹⁹F NMR (377 MHz, DMSO-d₆) δ −59.60, 62.83, 125.01.

### Step 3: Preparation of ethyl 2-[(14R*,17S)-14-{5-[2-(dimethylamino)ethyl]-2-oxo-4-(trifluoromethyl)pyridin-1-yl}-19-fluoro-3-methyl-15-oxo-20-(trifluoromethyl)-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate

To a stirred mixture of ethyl (3S)-3-[2-(3-bromophenyl)-2-{5-[2-(dimethylamino)ethyl]-2-oxo-4-(trifluoromethyl)pyridin-1-yl}acetamido]-3-[4-fluoro-2′-hydroxy-6′-methyl-5-(trifluoromethyl)-[1,1′-biphenyl]-3-yl]propanoate (340 mg, 0.41 mmol, 1.00 equiv) in 1,4-dioxane (6 mL) were added CuI (39 mg, 0.20 mmol, 0.50 equiv) and K₂CO₃ (115 mg, 0.83 mmol, 2.00 equiv) and (1R,2R)—N1,N2-dimethylcyclohexane-1,2-diamine (59 mg, 0.41 mmol, 1.00 equiv) at room temperature under nitrogen atmosphere. The resulting mixture was stirred overnight at 100° C. under nitrogen atmosphere. The mixture was allowed to cool down to room temperature. The reaction was monitored by LCMS. The resulting mixture was concentrated under reduced pressure. The residue was purified by reversed-phase flash chromatography (C18; mobile phase, MeCN in water (10 mmol/L NH₄HCO₃), 10% to 50% gradient in 10 min; detector, UV 254 nm/220 nm) to afford ethyl 2-[(14R*,17S)-14-{5-[2-(dimethylamino)ethyl]-2-oxo-4-(trifluoromethyl)pyridin-1-yl}-19-fluoro-3-methyl-15-oxo-20-(trifluoromethyl)-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (170 mg, 55.5%) as a yellow oil. LC-MS: (ES+H, m/z): [M+H]⁺=734.3.

### Step 3: Preparation of [(14R*,17S)-14-{5-[2-(dimethylamino)ethyl]-2-oxo-4-(trifluoromethyl)pyridin-1-yl}-19-fluoro-3-methyl-15-oxo-20-(trifluoromethyl)-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid

To a stirred mixture of ethyl 2-[(14R*,17S)-14-{5-[2-(dimethylamino)ethyl]-2-oxo-4-(trifluoromethyl)pyridin-1-yl}-19-fluoro-3-methyl-15-oxo-20-(trifluoromethyl)-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (170 mg, 0.23 mmol, 1.00 equiv) in THF (3 mL) was added LiOH·H₂O (11 mg, 0.46 mmol, 2.00 equiv) in H₂O (1 mL) at room temperature under nitrogen atmosphere. The resulting mixture was stirred for 2 h at room temperature under nitrogen atmosphere. The reaction was monitored by LCMS. Desired product could be detected by LCMS. The mixture was acidified to pH 4 with 1 M HCl (aq.). The resulting mixture was concentrated under reduced pressure to afford crude product. The crude product (170 mg) was purified by Prep-HPLC (Column: XBridge Shield RP18 OBD Column 30*150 mm, 5 μm; Mobile Phase A: Water (0.1% HCOOH), Mobile Phase B: MeCN; Flow rate: 60 mL/min; Gradient: 24% B to 39% B in 8 min; Wavelength: 254 nm/220 nm; RT1 (min): 9.95) to afford [(14R*,17S)-14-{5-[2-(dimethylamino)ethyl]-2-oxo-4-(trifluoromethyl)pyridin-1-yl}-19-fluoro-3-methyl-15-oxo-20-(trifluoromethyl)-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid (64.7 mg, 39.5%). LC-MS: (ES+H, m/z): [M+H]⁺=706.20. ¹H NMR (400 MHz, CD₃OD) δ 7.50-7.32 (m, 6H), 7.20 (d, J=7.4 Hz, 1H), 7.13 (d, J=7.3 Hz, 1H), 6.92 (s, 1H), 6.88 (s, 1H), 6.68 (s, 1H), 6.15 (t, J=2.0 Hz, 1H), 5.68 (s, 1H), 3.01 (d, J=27.2 Hz, 2H), 2.82-2.75 (m, 2H), 2.72 (d, J=2.2 Hz, 5H), 2.65 (s, 3H), 1.98 (s, 3H). ¹⁹F NMR (377 MHz, CD₃OD) δ −62.43, 64.90, 123.84.

### Example 17

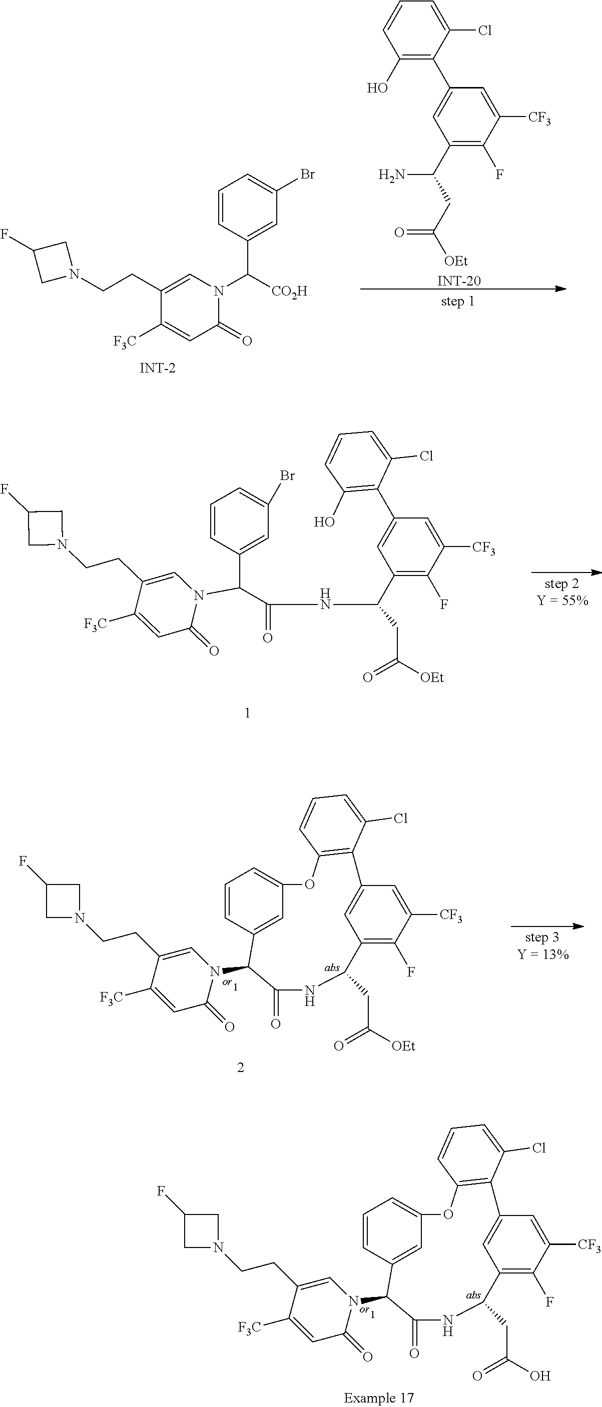

### Step 1: Preparation of ethyl (3S)-3-[2-(3-bromophenyl)-2-{5-[2-(3-fluoroazetidin-1-yl)ethyl]-2-oxo-4-(trifluoromethyl)pyridin-1-yl}acetamido]-3-[2′-chloro-4-fluoro-6′-hydroxy-5-(trifluoromethyl)-[1,1′-biphenyl]-3-yl]propanoate

A mixture of (3-bromophenyl)({5-[2-(3-fluoroazetidin-1-yl)ethyl]-2-oxo-4-(trifluoromethyl)pyridin-1-yl})acetic acid (300 mg, 0.62 mmol, 1.00 equiv), EDCI (193 mg, 1.00 mmol, 1.60 equiv) and HOBT (119 mg, 0.88 mmol, 1.40 equiv) in MeCN (6 mL) was stirred for 2 h at 30° C. under nitrogen atmosphere. To the stirred mixture was added a solution of ethyl (3S)-3-amino-3-[2′-chloro-4-fluoro-6′-hydroxy-5-(trifluoromethyl)-[1,1′-biphenyl]-3-yl]propanoate (280 mg, 0.63 mmol, 1.00 equiv) and NEt₃ (382 mg, 3.77 mmol, 6.00 equiv) in MeCN (4 mL) which was stirred for 30 min at 30° C. under nitrogen atmosphere. The resulting mixture was stirred overnight at 40° C. under nitrogen atmosphere. The reaction was monitored by LCMS. The resulting mixture was concentrated under reduced pressure. The residue was purified by reversed-phase flash chromatography (C18; mobile phase, MeCN in water (0.1% NH₃·H₂O), 30% to 60% gradient in 30 min; detector, UV 254 nm) to afford ethyl (3S)-3-[2-(3-bromophenyl)-2-{5-[2-(3-fluoroazetidin-1-yl)ethyl]-2-oxo-4-(trifluoromethyl)pyridin-1-yl}acetamido]-3-[2′-chloro-4-fluoro-6′-hydroxy-5-(trifluoromethyl)-[1,1′-biphenyl]-3-yl]propanoate (250 mg, 49.1% (over 2 steps)) as a light yellow solid. LC-MS: (ES+H, m/z): [M+H]⁺=864.0.

### Step 2: Preparation of ethyl 2-[(17S)-3-chloro-19-fluoro-14-{5-[2-(3-fluoroazetidin-1-yl)ethyl]-2-oxo-4-(trifluoromethyl)pyridin-1-yl}-15-oxo-20-(trifluoromethyl)-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate

To a stirred solution of ethyl (3S)-3-[2-(3-bromophenyl)-2-{5-[2-(3-fluoroazetidin-1-yl)ethyl]-2-oxo-4-(trifluoromethyl)pyridin-1-yl}acetamido]-3-[2′-chloro-4-fluoro-6′-hydroxy-5-(trifluoromethyl)-[1,1′-biphenyl]-3-yl]propanoate (200 mg, 0.23 mmol, 1.00 equiv) in dioxane (15 mL) were added CuI (22 mg, 0.12 mmol, 0.50 equiv), K₂CO₃ (64 mg, 0.46 mmol, 2.00 equiv) and N₁,N₂-dimethylcyclohexane-1,2-diamine (33 mg, 0.23 mmol, 1.00 equiv) in portions at room temperature under nitrogen atmosphere. The resulting mixture was stirred overnight at 100° C. under nitrogen atmosphere and then cooled to room temperature. The resulting mixture was concentrated under reduced pressure. The residue was purified by silica gel column chromatography, eluted with CH₂Cl₂/MeOH (0% to 5% gradient in 20 min) to afford ethyl 2-[(17S)-3-chloro-19-fluoro-14-{5-[2-(3-fluoroazetidin-1-yl)ethyl]-2-oxo-4-(trifluoromethyl)pyridin-1-yl}-15-oxo-20-(trifluoromethyl)-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (100 mg, 55.1%) as a yellow solid. LC-MS: (ES+H, m/z): [M+H]⁺=784.1.

### Step 3: Preparation of [(14R*,17S)-3-chloro-19-fluoro-14-{5-[2-(3-fluoroazetidin-1-yl)ethyl]-2-oxo-4-(trifluoromethyl)pyridin-1-yl}-15-oxo-20-(trifluoromethyl)-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid

To a stirred solution of ethyl 2-[(17S)-3-chloro-19-fluoro-14-{5-[2-(3-fluoroazetidin-1-yl)ethyl]-2-oxo-4-(trifluoromethyl)pyridin-1-yl}-15-oxo-20-(trifluoromethyl)-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (100 mg, 0.13 mmol, 1.00 equiv) in THF (5 mL) was added LiOH·H₂O (6 mg, 0.26 mmol, 2.00 equiv) in H₂O (0.5 mL) dropwise at 0° C. under nitrogen atmosphere. The resulting mixture was stirred for 2 h at room temperature under nitrogen atmosphere. The reaction was monitored by LCMS. The residue was acidified to pH 6 with 1 M HCl (aq.). The resulting mixture was concentrated under reduced pressure. The crude product (100 mg) was purified by Prep-HPLC (Column: Xbridge Phenyl OBD Column, 19*150 mm, 5 μm; Mobile Phase A: Water (10 mmol/L NH₄HCO₃+0.05% NH₃H₂O, Mobile Phase B: MeCN; Flow rate: 60 mL/min; Gradient: 27% B to 42% B in 8 min; Wavelength: 254 nm/220 nm; RT1 (min): 8.18) to afford [(14R*,17S)-3-chloro-19-fluoro-14-{5-[2-(3-fluoroazetidin-1-yl)ethyl]-2-oxo-4-(trifluoromethyl)pyridin-1-yl}-15-oxo-20-(trifluoromethyl)-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid (12.7 mg, 13.1%). LC-MS: (ES+H, m/z): [M+H]⁺=756.2. ¹H NMR (400 MHz, CD₃OD) δ 7.67 (dd, J=8.1, 1.2 Hz, 1H), 7.56-7.42 (m, 5H), 7.38 (s, 1H), 7.18 (dt, J=7.2, 1.6 Hz, 1H), 6.92-6.84 (m, 2H), 6.64 (s, 1H), 6.17 (t, J=2.0 Hz, 1H), 5.69 (t, J=7.2 Hz, 1H), 5.10-4.89 (m, 1H), 3.70-3.57 (m, 2H), 3.33 (d, J=5.5 Hz, 1H), 3.25 (dd, J=9.5, 4.9 Hz, 1H), 2.71 (dt, J=14.0, 4.6 Hz, 4H), 2.53-2.44 (m, 2H). ¹⁹F NMR (377 MHz, CD₃OD) δ −62.54, −64.91, −122.56, −180.39.

### Example 18

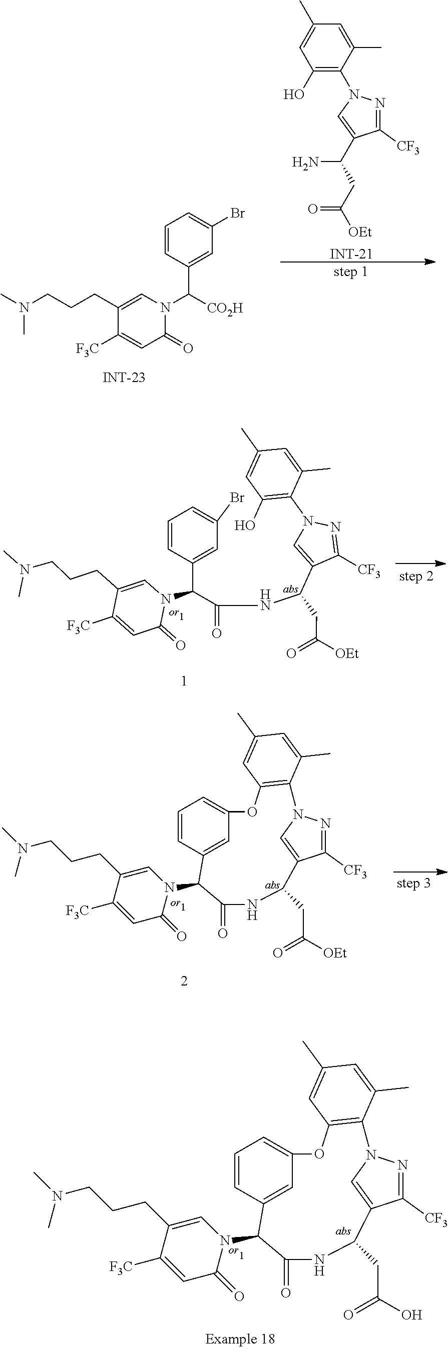

### Step 1: Preparation of ethyl (3S)-3-[2-(3-bromophenyl)-2-{5-[3-(dimethylamino)propyl]-2-oxo-4-(trifluoromethyl)pyridin-1-yl}acetamido]-3-[1-(2-hydroxy-4,6-dimethylphenyl)-3-(trifluoromethyl)pyrazol-4-yl]propanoate

A mixture of (3-bromophenyl)({5-[3-(dimethylamino)propyl]-2-oxo-4-(trifluoromethyl)pyridin-1-yl})acetic acid (400 mg, 0.86 mmol, 1.00 equiv.), HOBT (152 mg, 1.12 mmol, 1.30 equiv.) and EDCI (249 mg, 1.30 mmol, 1.50 equiv.) in CH₃CN (7 mL) was stirred for 1 h at room temperature under nitrogen atmosphere. To the above mixture was added ethyl (3S)-3-amino-3-[1-(2-hydroxy-4,6-dimethylphenyl)-3-(trifluoromethyl)pyrazol-4-yl]propanoate (322 mg, 0.86 mmol, 1.00 equiv.), TEA (263 mg, 2.60 mmol, 3.00 equiv.) in CH₃CN (3 mL) dropwise over 5 min at room temperature. The resulting mixture was stirred overnight at room temperature. The resulting mixture was diluted with EtOAc (100 mL) and washed with water (2×20 mL), dried over anhydrous Na₂SO₄, filtered, and concentrated. The residue was purified by reversed-phase flash chromatography (C18 gel; mobile phase, MeCN in water (0.1% NH₃·H₂O), 0% to 70% gradient in 40 min; detector, UV 220 nm) to provide ethyl (3S)-3-[2-(3-bromophenyl)-2-{5-[3-(dimethylamino)propyl]-2-oxo-4-(trifluoromethyl)pyridin-1-yl}acetamido]-3-[1-(2-hydroxy-4,6-dimethylphenyl)-3-(trifluoromethyl)pyrazol-4-yl]propanoate (240 mg, crude) as a brown solid. LC-MS: (ES+H, m/z) [M+H]⁺=814.1.

### Step 2: Preparation of ethyl 2-[(13S)-16-{5-[3-(dimethylamino)propyl]-2-oxo-4-(trifluoromethyl)pyridin-1-yl}-5,7-dimethyl-15-oxo-11-(trifluoromethyl)-2-oxa-9,10,14-triazatetracyclo[15.3.1.1\hat{ }{9,12}.0\hat{ }{3,8}]docosa-1(20),3(8),4,6,10,12(22),17(21),18-octaen-13-yl]acetate

To a stirred mixture of ethyl (3S)-3-[2-(3-bromophenyl)-2-{5-[3-(dimethylamino)propyl]-2-oxo-4-(trifluoromethyl)pyridin-1-yl}acetamido]-3-[1-(2-hydroxy-4,6-dimethylphenyl)-3-(trifluoromethyl)pyrazol-4-yl]propanoate (240 mg, crude) and (1R,2R)—N1,N2-dimethylcyclohexane-1,2-diamine (42 mg, 0.30 mmol, 1.00 equiv.) in dioxane (10 mL) were added CuI (28 mg, 0.15 mmol, 0.50 equiv.) and K₂CO₃ (122 mg, 0.90 mmol, 3.00 equiv.) in portions at room temperature under nitrogen atmosphere. The resulting mixture was stirred overnight at 100° C. under nitrogen atmosphere and then cooled to room temperature and diluted with EtOAc (100 mL). The reaction mixture was washed with water (2×20 mL), dried over anhydrous Na₂SO₄, filtered, and concentrated. The residue was purified by reversed-phase flash chromatography (C18 gel; mobile phase, MeCN in water (0.1% NH₃·H₂O), 0% to 70% gradient in 40 min; detector, UV 220 nm) to provide ethyl 2-[(13S)-16-{5-[3-(dimethylamino)propyl]-2-oxo-4-(trifluoromethyl)pyridin-1-yl}-5,7-dimethyl-15-oxo-11-(trifluoromethyl)-2-oxa-9,10,14-triazatetracyclo[15.3.1.1\hat{ }{9,12}.0\hat{ }{3,8}]docosa-1(20),3(8),4,6,10,12(22),17(21),18-octaen-13-yl]acetate (160 mg, crude) as a brown solid. LC-MS: (ES+H, m/z) [M+H]⁺=734.3.

### Step 3: Preparation of [(13S,16R*)-16-{5-[3-(dimethylamino)propyl]-2-oxo-4-(trifluoromethyl)pyridin-1-yl}-5,7-dimethyl-15-oxo-11-(trifluoromethyl)-2-oxa-9,10,14-triazatetracyclo[15.3.1.1\hat{ }{9,12}.0\hat{ }{3,8}]docosa-1(20),3(8),4,6,10,12(22),17(21),18-octaen-13-yl]acetic acid

To a stirred solution of ethyl 2-[(13S)-16-{5-[3-(dimethylamino)propyl]-2-oxo-4-(trifluoromethyl)pyridin-1-yl}-5,7-dimethyl-15-oxo-11-(trifluoromethyl)-2-oxa-9,10,14-triazatetracyclo[15.3.1.1\hat{ }{9,12}.0\hat{ }{3,8}]docosa-1(20),3(8),4,6,10,12(22),17(21),18-octaen-13-yl]acetate (150 mg, crude) in THF (2 mL) were added H₂O (0.4 mL) and LiOH·H₂O (43 mg, 1.02 mmol, 5.00 equiv.) in portions at room temperature under nitrogen atmosphere. The resulting mixture was stirred for 2 h at room temperature and then neutralized to pH 7 with 1 M HCl (aq.) and concentrated. The residue was purified by reverse flash chromatography ((Column: XBridge Shield RP18 OBD Column, 50*250 mm, 10 μm; Mobile Phase A: water (10 mmol/L NH₄OH), Mobile Phase B: CH₃CN; Flow rate: 100 mL/min; Gradient: 20% B to 60% B in 40 min, 50% B; Wavelength: 254/220 nm; RT1 (min) 30) to afford [(13S,16R*)-16-{5-[3-(dimethylamino)propyl]-2-oxo-4-(trifluoromethyl)pyridin-1-yl}-5,7-dimethyl-15-oxo-11-(trifluoromethyl)-2-oxa-9,10,14-triazatetracyclo[15.3.1.1\hat{ }{9,12}.0\hat{ }{3,8}]docosa-1(20),3(8),4,6,10,12(22),17(21),18-octaen-13-yl]acetic acid (42.8 mg) as a white solid. LC-MS: (ES+H, m/z) [M+H]⁺=706.25. ¹H NMR (400 MHz, CD₃OD) δ 7.43 (t, J=8.0 Hz, 1H), 7.32 (d, J=6.2 Hz, 2H), 7.23-7.20 (m, 2H), 7.14 (s, 1H), 6.89 (d, J=6.1 Hz, 1H), 6.69 (s, 1H), 6.63-6.61 (m, 1H), 6.21 (t, J=2.0 Hz, 1H), 5.42 (t, J=6.0 Hz, 1H), 2.94 (dt, J=9.7, 4.1 Hz, 2H), 2.74-2.65 (m, 7H), 2.56 (dd, J=15.5, 6.2 Hz, 2H), 2.47-2.40 (m, 4H), 1.98 (s, 3H), 1.90-1.76 (m, 2H). ¹⁹F NMR (377 MHz, CD₃OD) δ −60.82, −64.90.

### Example 19

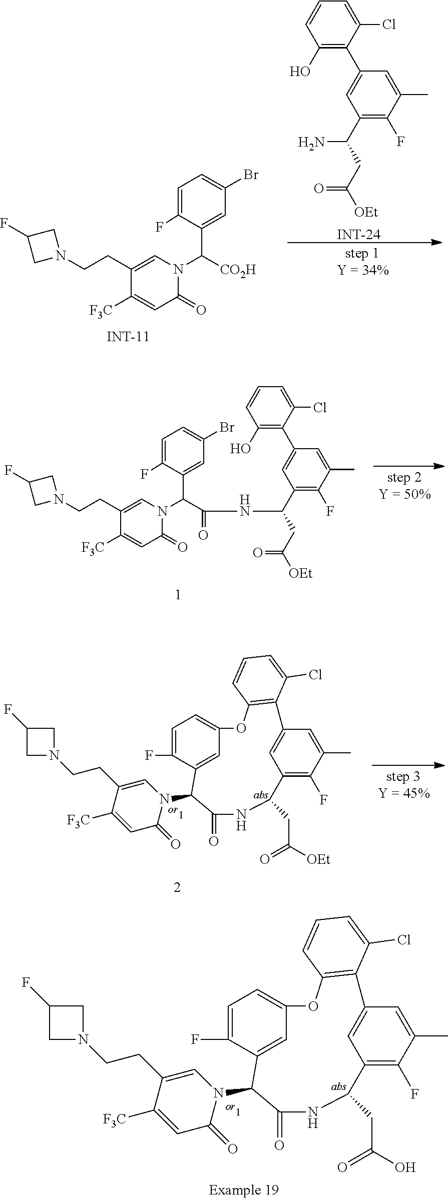

### Step 1: Preparation of ethyl (3S)-3-[2-(5-bromo-2-fluorophenyl)-2-{5-[2-(3-fluoroazetidin-1-yl)ethyl]-2-oxo-4-(trifluoromethyl)pyridin-1-yl}acetamido]-3-{2′-chloro-4-fluoro-6′-hydroxy-5-methyl-[1,1′-biphenyl]-3-yl}propanoate

To a stirred solution of (5-bromo-2-fluorophenyl)({5-[2-(3-fluoroazetidin-1-yl)ethyl]-2-oxo-4-(trifluoromethyl)pyridin-1-yl})acetic acid (300 mg, 0.60 mmol, 1.00 equiv.) in MeCN (8.00 mL) was added HOBT (106 mg, 0.78 mmol, 1.30 equiv.) and EDCI (174 mg, 0.90 mmol, 1.50 equiv.) in MeCN (8.00 mL) at room temperature under nitrogen atmosphere. The resulting mixture was stirred for 1 h (solution 1). In a separate vial, ethyl (3S)-3-amino-3-{2′-chloro-4-fluoro-6′-hydroxy-5-methyl-[1,1′-biphenyl]-3-yl}propanoate hydrochloride (258 mg, 0.66 mmol, 1.10 equiv., assumed 100% yield) in MeCN (2.00 mL) was treated with Et₃N (306 mg, 3.03 mmol, 5.00 equiv.) at room temperature and then concentrated and dissolved in MeCN (2.00 mL, solution 2). To the above mixture (solution 1) was added (solution 2) at room temperature. The reaction mixture was stirred overnight at room temperature and then extracted with EtOAc (3×100 mL). The combined organic layers were washed with brine (3×30 mL), dried over anhydrous Na₂SO₄, filtered, and concentrated. The crude product was purified by reversed-phase flash chromatography (C18 gel; mobile phase, MeCN in water (10 mmol/L NH₄HCO₃), 10% to 90% gradient in 40 min; detector, UV 254 nm) to provide ethyl (3S)-3-[2-(5-bromo-2-fluorophenyl)-2-{5-[2-(3-fluoroazetidin-1-yl)ethyl]-2-oxo-4-(trifluoromethyl)pyridin-1-yl}acetamido]-3-{2′-chloro-4-fluoro-6′-hydroxy-5-methyl-[1,1′-biphenyl]-3-yl}propanoate (180 mg, 34.0%) as a white solid. LC-MS: (ES+H, m/z) [M+H]⁺=828.1. ¹H NMR (400 MHz, CD₃OD) δ 7.77-7.55 (m, 2H), 7.40-7.36 (m, 1H), 7.18-7.04 (m, 4H), 6.98-6.91 (m, 2H), 6.86-6.76 (m, 2H), 5.75-5.67 (m, 1H), 5.07-4.89 (m, 1H), 4.16-4.05 (m, 2H), 3.59-3.38 (m, 2H), 3.11-3.01 (m, 2H), 2.94 (dd, J=7.6, 3.9 Hz, 1H), 2.87 (d, J=7.3 Hz, 1H), 2.63-2.45 (m, 4H), 2.31 (dd, J=10.3, 2.0 Hz, 3H), 1.16 (td, J=7.1, 2.9 Hz, 3H).

### Step 2: Preparation of ethyl 2-[(14R*,17S)-3-chloro-12,19-difluoro-14-{5-[2-(3-fluoroazetidin-1-yl)ethyl]-2-oxo-4-(trifluoromethyl)pyridin-1-yl}-20-methyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate

To a stirred solution of ethyl (3S)-3-[2-(5-bromo-2-fluorophenyl)-2-{5-[2-(3-fluoroazetidin-1-yl)ethyl]-2-oxo-4-(trifluoromethyl)pyridin-1-yl}acetamido]-3-{2′-chloro-4-fluoro-6′-hydroxy-5-methyl-[1,1′-biphenyl]-3-yl}propanoate (180 mg, 0.21 mmol, 1.00 equiv.) and (1R,2R)—N1,N2-dimethylcyclohexane-1,2-diamine (30 mg, 0.21 mmol, 1.00 equiv.) in 1,4-dioxane (3.00 mL) was added CuI (20 mg, 0.10 mmol, 0.50 equiv.) at room temperature under nitrogen atmosphere. The resulting mixture was stirred overnight at 100° C. under nitrogen atmosphere and then cooled to room temperature and concentrated. The residue was purified by reversed-phase flash chromatography (C18 gel; mobile phase, MeCN in water (10 mmol/L NH₄HCO₃), 10% to 90% gradient in 40 min; detector, UV 254 nm) to provide ethyl 2-[(14R*,17S)-3-chloro-12,19-difluoro-14-{5-[2-(3-fluoroazetidin-1-yl)ethyl]-2-oxo-4-(trifluoromethyl)pyridin-1-yl}-20-methyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (85 mg, 49.7%) as a yellow solid. LC-MS: (ES+H, m/z) [M+H]⁺=748.2. ¹H NMR (400 MHz, CD₃OD) δ 7.64 (dd, J=8.0, 1.3 Hz, 1H), 7.59 (ddd, J=9.1, 4.0, 3.0 Hz, 1H), 7.47 (t, J=8.1 Hz, 1H), 7.41 (dd, J=8.1, 1.3 Hz, 1H), 7.35 (s, 1H), 7.20 (t, J=9.0 Hz, 1H), 7.04 (dd, J=7.1, 2.2 Hz, 1H), 6.84 (d, J=20.0 Hz, 2H), 6.47 (dd, J=6.2, 2.2 Hz, 1H), 6.12 (dd, J=6.1, 3.0 Hz, 1H), 5.71 (dd, J=8.5, 7.1 Hz, 1H), 5.01-4.80 (m, 1H), 4.16-4.08 (m, 2H), 3.48-3.35 (m, 2H), 3.09-2.95 (m, 2H), 2.88-2.70 (m, 2H), 2.62-2.37 (m, 4H), 2.35 (d, J=2.0 Hz, 3H), 1.20 (t, J=7.1 Hz, 3H).

### Step 3: Preparation of [(14R*,17S)-3-chloro-12,19-difluoro-14-{5-[2-(3-fluoroazetidin-1-yl)ethyl]-2-oxo-4-(trifluoromethyl)pyridin-1-yl}-20-methyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid

To a stirred solution of ethyl 2-[(14R*,17S)-3-chloro-12,19-difluoro-14-{5-[2-(3-fluoroazetidin-1-yl)ethyl]-2-oxo-4-(trifluoromethyl)pyridin-1-yl}-20-methyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (85 mg, 0.11 mmol, 1.00 equiv.) in THF (1.00 mL) and H₂O (0.25 mL) was added LiOH·H₂O (23 mg, 0.57 mmol, 5.00 equiv.) at room temperature under nitrogen atmosphere. The resulting mixture was stirred for 2 h at room temperature and then neutralized to pH 7 with 1 M HCl (aq.), and concentrated. The residue was dissolved in DMSO (2.00 mL) and the crude product was purified by Prep-HPLC (Column: XSelect CSH Prep C18 OBD Column, 30*150 mm, 5 μm; Mobile Phase A: water (0.1% HCOOH), Mobile Phase B: MeCN; Flow rate: 60 mL/min; Gradient: 17% B to 37% B in 10 min; Wavelength: 254 nm/220 nm; RT1 (min) 9.83)) to afford [(14R*,17S)-3-chloro-12,19-difluoro-14-{5-[2-(3-fluoroazetidin-1-yl)ethyl]-2-oxo-4-(trifluoromethyl)pyridin-1-yl}-20-methyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid (37.9 mg, 45.4%) as a white solid. LC-MS: (ES+H, m/z) [M+H]⁺=720.20. ¹H NMR (400 MHz, CD₃OD) δ 7.64-7.57 (m, 2H), 7.48-7.39 (m, 2H), 7.34 (s, 1H), 7.19 (t, J=9.0 Hz, 1H), 7.04-7.02 (m, 1H), 6.88 (s, 2H), 6.49 (dd, J=6.3, 2.2 Hz, 1H), 6.14 (dd, J=6.1, 3.0 Hz, 1H), 5.72 (dd, J=8.5, 6.5 Hz, 1H), 5.11-4.91 (m, 1H), 3.71-3.60 (m, 2H), 3.39-3.33 (m, 1H), 3.31-3.30 (m, 1H), 2.77-2.69 (m, 4H), 2.51-2.49 (m, 2H), 2.35 (d, J=2.1 Hz, 3H). ¹⁹F NMR (377 MHz, CD₃OD) δ −64.99, −122.47, −125.64, −180.65.

### Example 20

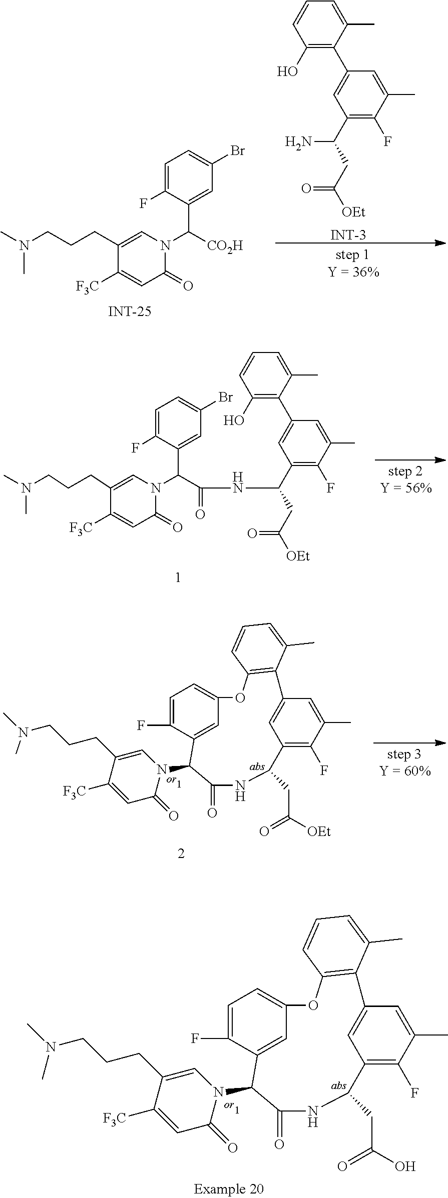

### Step 1: Preparation of ethyl (3S)-3-[2-(5-bromo-2-fluorophenyl)-2-{5-[3-(dimethylamino)propyl]-2-oxo-4-(trifluoromethyl)pyridin-1-yl}acetamido]-3-{4-fluoro-2′-hydroxy-5,6′-dimethyl-[1,1′-biphenyl]-3-yl}propanoate

A solution of ethyl (3S)-3-amino-3-{4-fluoro-2′-hydroxy-5,6′-dimethyl-[1,1′-biphenyl]-3-yl}propanoate hydrochloride (324 mg, assumed 100% yield, 0.98 mmol, 1.00 equiv.) and triethylamine (496 mg, 4.90 mmol, 5.00 equiv.) in MeCN (5 mL) was stirred for 1 h at room temperature under nitrogen atmosphere and then concentrated. In a separate flask, a stirred solution of (5-bromo-2-fluorophenyl)({5-[3-(dimethylamino)propyl]-2-oxo-4-(trifluoromethyl)pyridin-1-yl})acetic acid (470 mg, 0.98 mmol, 1.00 equiv.) in MeCN (5 mL) was treated with HOBT (198 mg, 1.47 mmol, 1.50 equiv.) and EDCI (375 mg, 1.96 mmol, 2.00 equiv.) at room temperature under nitrogen atmosphere. The resulting mixture was stirred for an additional 1 h at room temperature and then ethyl (3S)-3-amino-3-{4-fluoro-2′-hydroxy-5,6′-dimethyl-[1,1′-biphenyl]-3-yl}propanoate in MeCN (3 mL) was added dropwise at 0° C. The resulting mixture was stirred overnight at 40° C. and then concentrated. The residue was purified by reversed-phase flash chromatography (C18 gel; mobile phase, MeCN in water (0.1% NH₃·H₂O), 40% to 80% gradient in 20 min; detector, UV 254 nm) to provide ethyl (3S)-3-[2-(5-bromo-2-fluorophenyl)-2-{5-[3-(dimethylamino)propyl]-2-oxo-4-(trifluoromethyl)pyridin-1-yl}acetamido]-3-{4-fluoro-2′-hydroxy-5,6′-dimethyl-[1,1′-biphenyl]-3-yl}propanoate (280 mg, 36.0%) as light yellow solid. LC-MS: (ES+H, m/z) [M+H]⁺=792.15.

### Step 2: Preparation of ethyl 2-[(14R*,17S)-14-{5-[3-(dimethylamino)propyl]-2-oxo-4-(trifluoromethyl)pyridin-1-yl}-12,19-difluoro-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate

To a stirred solution of ethyl (3S)-3-[2-(5-bromo-2-fluorophenyl)-2-{5-[3-(dimethylamino)propyl]-2-oxo-4-(trifluoromethyl)pyridin-1-yl}acetamido]-3-{4-fluoro-2′-hydroxy-5,6′-dimethyl-[1,1′-biphenyl]-3-yl}propanoate (250 mg, 0.31 mmol, 1.00 equiv.) in 1,4-dioxane (5 mL) were added CuI (30 mg, 0.15 mmol, 0.50 equiv.), K₂CO₃ (87. mg, 0.63 mmol, 2.00 equiv.) and (1R,2R)—N1, N2-dimethylcyclohexane-1,2-diamine (44 mg, 0.31 mmol, 1.00 equiv.) at room temperature under nitrogen atmosphere. The resulting mixture was stirred overnight at 100° C. and then cooled to room temperature and concentrated. The residue was purified by reversed-phase flash chromatography (C18 gel; mobile phase, MeCN in water (0.1% NH₃·H₂O), 10% to 50% gradient in 10 min; detector, UV 254 nm) to provide ethyl 2-[(14R*,17S)-14-{5-[3-(dimethylamino)propyl]-2-oxo-4-(trifluoromethyl)pyridin-1-yl}-12,19-difluoro-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (125 mg, 55.6%) as light yellow solid. LC-MS: (ES+H, m/z) [M+H]⁺=712.25. ¹H NMR (400 MHz, CDCl₃) δ7.68 (d, J=7.7 Hz, 1H), 7.40-7.30 (m, 3H), 7.14 (dd, J=6.3, 2.5 Hz, 1H), 7.06 (d, J=8.7 Hz, 1H), 6.99 (s, 1H), 6.95 (d, J=5.7 Hz, 2H), 6.87 (s, 1H), 6.45-6.34 (m, 2H), 5.76 (q, J=6.1 Hz, 1H), 4.12-3.94 (m, 2H), 2.93-2.77 (m, 2H), 2.39 (t, J=7.7 Hz, 2H), 2.34 (d, J=1.8 Hz, 3H), 2.23 (d, J=10.2 Hz, 2H), 2.10 (s, 6H), 1.98 (s, 3H), 1.62-1.47 (m, 2H), 1.13 (t, J=7.1 Hz, 3H). ¹⁹F NMR (377 MHz, CDCl₃) −63.80, −120.90, −124.96.

### Step 3: Preparation of [(14R*,17S)-14-{5-[3-(dimethylamino)propyl]-2-oxo-4-(trifluoromethyl)pyridin-1-yl}-12,19-difluoro-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid

To a stirred solution of ethyl 2-[(14R*,17S)-14-{5-[3-(dimethylamino)propyl]-2-oxo-4-(trifluoromethyl)pyridin-1-yl}-12,19-difluoro-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (120 mg, 0.16 mmol, 1.00 equiv.) in THF (4 mL) and H₂O (1 mL) were added LiOH·H₂O (35 mg, 0.84 mmol, 5.00 equiv.) at room temperature under nitrogen atmosphere. After 2 h, the reaction was acidified to pH 5 with HCl (aq. 1 M) and then concentrated. The crude product (120 mg) was purified by Prep-HPLC (Column: XSelect CSH Fluoro Phenyl, 30*150 mm, 5 μm; Mobile Phase A: water (0.1% HCOOH), Mobile Phase B: 20 mm NaOH+10% MeCN; Flow rate: 60 mL/min; Gradient: 12% B to 32% B in 10 min; Wavelength: 254 nm/220 nm; RT1 (min) 10.62)) to afford [(14R*,17S)-14-{5-[3-(dimethylamino)propyl]-2-oxo-4-(trifluoromethyl)pyridin-1-yl}-12,19-difluoro-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid (72.7 mg, 59.9%) as a white solid. LC-MS: (ES+H, m/z) [M+H]⁺=684.30. ¹H NMR (400 MHz, CD₃OD) δ 8.46-8.37 (m, 1H), 7.57-7.51 (m, 1H), 7.42 (d, J=7.6 Hz, 1H), 7.33 (t, J=7.8 Hz, 1H), 7.18-7.11 (m, 3H), 7.01-6.94 (m, 2H), 6.90 (s, 1H), 6.51 (dd, J=6.4, 2.2 Hz, 1H), 6.14 (dd, J=6.2, 2.9 Hz, 1H), 5.68 (dd, J=8.0, 6.9 Hz, 1H), 3.14-2.97 (m, 2H), 2.80 (s, 6H), 2.76-2.55 (m, 3H), 2.46-2.35 (m, 1H), 2.34 (d, J=1.8 Hz, 3H), 2.00 (s, 3H), 1.91-1.71 (m, 2H). ¹⁹F NMR (377 MHz, CD₃OD) −64.947, −122.917, −127.036.

### Example 21

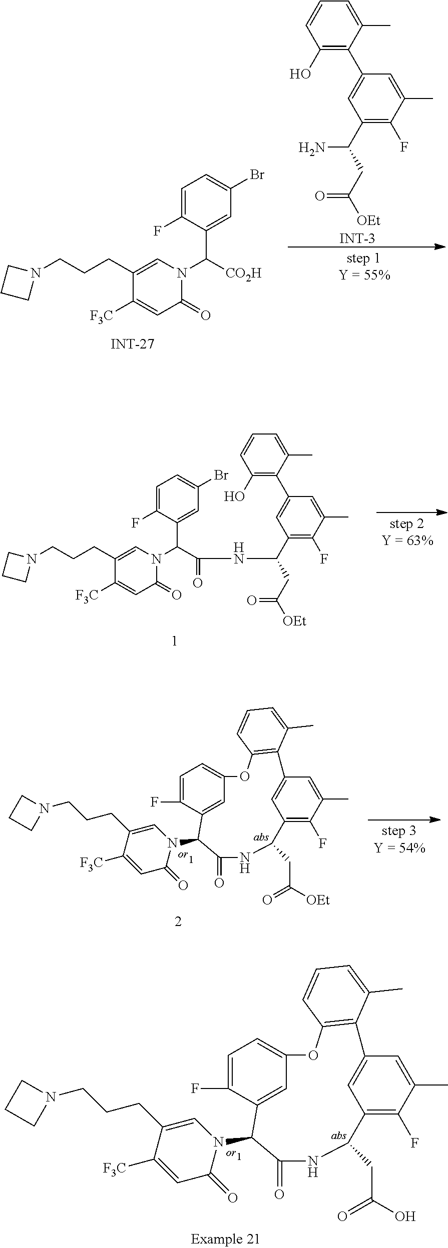

### Step 1: Preparation of ethyl (3S)-3-(2-(5-(3-(azetidin-1-yl) propyl)-2-oxo-4-(trifluoromethyl) pyridin-1(2H)-yl)-2-(5-bromo-2-fluorophenyl) acetamido)-3-(4-fluoro-2′-hydroxy-5,6′-dimethyl-[1,1′-biphenyl]-3-yl) propanoate

A solution of ethyl (3S)-3-amino-3-{4-fluoro-2′-hydroxy-5,6′-dimethyl-[1,1′-biphenyl]-3-yl} propanoate (500 mg crude, 0.56 mmol, 1.00 equiv., assumed 100% yield) and NEt₃ (170 mg, 1.68 mmol, 3.00 equiv.) in MeCN (3 mL) was stirred for 1 h at room temperature under nitrogen atmosphere. In a separate vial, a solution of {5-[3-(azetidin-1-yl) propyl]-2-oxo-4-(trifluoromethyl) pyridin-1-yl}(5-bromo-2-fluorophenyl) acetic acid (276 mg, 0.56 mmol, 1.00 equiv.), HOBT (113 mg, 0.84 mmol, 1.50 equiv.) and EDCI (215 mg, 1.12 mmol, 2.00 equiv.) in MeCN (5 mL) was stirred for 1 h at 40° C. under nitrogen atmosphere. To the above mixture was added the solution of ethyl (3S)-3-amino-3-{4-fluoro-2′-hydroxy-5,6′-dimethyl-[1,1′-biphenyl]-3-yl}propanoate (186 mg, 0.56 mmol, 1.00 equiv.) dropwise over 5 min at 0° C. and then stirred overnight at 40° C. The reaction was then concentrated and purified by reversed-phase flash chromatography (C18; mobile phase, MeCN in water (0.1% NH₃·H₂O), 30% to 70% gradient in 15 min; detector, UV 220 nm) to provide ethyl (3S)-3-(2-{5-[3-(azetidin-1-yl) propyl]-2-oxo-4-(trifluoromethyl) pyridin-1-yl}-2-(5-bromo-2-fluorophenyl) acetamido)-3-{4-fluoro-2′-hydroxy-5,6′-dimethyl-[1,1′-biphenyl]-3-yl}propanoate (250 mg, 55.3%) as off-white solid. LC-MS: (ES+H, m/z) [M+H]⁺=804.1.

### Step 2: Preparation of ethyl 2-[(14S,17S)-14-{5-[3-(azetidin-1-yl) propyl]-2-oxo-4-(trifluoromethyl) pyridin-1-yl}-12,19-difluoro-3,20-dimethyl-15-oxo-8-oxa-16-azatetracyclo [16.3.1.1\hat{ }{9,13}.0\hat{ }{2,7}] tricosa-1(22),2(7),3,5,9,11,13(23),18,20-nonaen-17-yl] acetate

To a stirred solution of ethyl (3S)-3-(2-{5-[3-(azetidin-1-yl) propyl]-2-oxo-4-(trifluoromethyl) pyridin-1-yl}-2-(5-bromo-2-fluorophenyl) acetamido)-3-{4-fluoro-2′-hydroxy-5,6′-dimethyl-[1,1′-biphenyl]-3-yl} propanoate (230 mg, 0.28 mmol, 1.00 equiv.) in 1,4-dioxane (6 mL) were added CuI (27 mg, 0.14 mmol, 0.50 equiv.), (1R,2R)—N1, N2-dimethylcyclohexane-1,2-diamine (41 mg, 0.28 mmol, 1.00 equiv.) and K₂CO₃ (79 mg, 0.57 mmol, 2.00 equiv.) at room temperature under nitrogen atmosphere. The resulting mixture was stirred overnight at 100° C. and then concentrated. The residue was purified by reversed-phase flash chromatography (C18; mobile phase, MeCN in water (0.1% NH₃·H₂O), 10% to 50% gradient in 20 min; detector, UV 220 nm) to afford ethyl 2-[(14S,17S)-14-{5-[3-(azetidin-1-yl) propyl]-2-oxo-4-(trifluoromethyl) pyridin-1-yl}-12,19-difluoro-3,20-dimethyl-15-oxo-8-oxa-16-azatetracyclo [16.3.1.1\hat{ }{9,13}.0\hat{ }{2,7}] tricosa-1(22),2(7),3,5,9,11,13(23),18,20-nonaen-17-yl] acetate (130 mg, 62.84%) as off-white solid. LC-MS: (ES+H, m/z) [M+H]⁺=724.2. ¹H NMR (300 MHz, CDCl₃) δ 7.59 (s, 1H), 7.46-7.32 (m, 3H), 7.21-7.03 (m, 2H), 7.03-6.85 (m, 4H), 6.43 (d, J=7.3 Hz, 2H), 5.79 (s, 1H), 4.12-3.95 (m, 2H), 3.14-2.93 (m, 4H), 2.93-2.75 (m, 2H), 2.45-2.18 (m, 7H), 2.00 (s, 5H), 1.39 (t, J=7.8 Hz, 2H), 1.15 (t, J=7.1 Hz, 3H). ¹⁹F NMR (282 MHz, CDCl₃) δ −63.85, −120.93, −124.95.

### Step 3: Preparation of 2-((5S,8S)-5-(5-(3-(azetidin-1-yl)propyl)-2-oxo-4-(trifluoromethyl)pyridin-1(2H)-yl)-14,44-difluoro-15,26-dimethyl-6-oxo-3-oxa-7-aza-1,4(1,3),2(1,2)-tribenzenacyclooctaphane-8-yl)acetic acid

To a stirred solution of ethyl 2-[(14S,17S)-14-{5-[3-(azetidin-1-yl) propyl]-2-oxo-4-(trifluoromethyl) pyridin-1-yl}-12,19-difluoro-3,20-dimethyl-15-oxo-8-oxa-16-azatetracyclo [16.3.1.1\hat{ }{9,13}.0\hat{ }{2,7}]tricosa-1(22),2(7),3,5,9,11,13(23),18,20-nonaen-17-yl] acetate (110 mg, 0.15 mmol, 1.00 equiv.) in THF:H₂O=5:1 (6 mL) was added LiOH·H₂O (14 mg, 0.60 mmol, 4.00 equiv.) at room temperature under nitrogen atmosphere. The resulting mixture was stirred for 2 h at 30° C. and then acidified to pH 5 with 1 M. HCl (aq.). The resulting mixture was concentrated under reduced pressure and the residue was purified by reversed-phase flash chromatography (C18; mobile phase, MeCN in water (0.1% HCOOH), 10% to 50% gradient in 20 min; detector, UV 220 nm) to afford 2-((5S,8S)-5-(5-(3-(azetidin-1-yl)propyl)-2-oxo-4-(trifluoromethyl)pyridin-1(2H)-yl)-14,44-difluoro-15,26-dimethyl-6-oxo-3-oxa-7-aza-1,4(1,3),2(1,2)-tribenzenacyclooctaphane-8-yl)acetic acid (57.3 mg, 53.6%) as a white solid. LC-MS: (ES+H, m/z) [M+H]⁺=696.10. ¹H NMR (400 MHz, CD₄OD) δ 8.40 (s, 1H), 7.59-7.50 (m, 1H), 7.42 (d, J=8.0 Hz, 1H), 7.33 (t, J=7.8 Hz, 1H), 7.21-7.09 (m, 3H), 6.96 (d, J=7.3 Hz, 2H), 6.90 (s, 1H), 6.51 (d, J=5.9 Hz, 1H), 6.22-6.05 (m, 1H), 5.69 (t, J=7.4 Hz, 1H), 4.05 (s, 4H), 3.20-3.01 (m, 2H), 2.79-2.60 (m, 2H), 2.59-2.50 (m, 1H), 2.50-2.36 (m, 3H), 2.33 (d, J=1.8 Hz, 3H), 2.00 (s, 3H), 1.72-1.55 (m, 2H). ¹⁹F NMR (377 MHz, CD₄OD) δ −64.93, −122.92, −127.03.

### Example 22

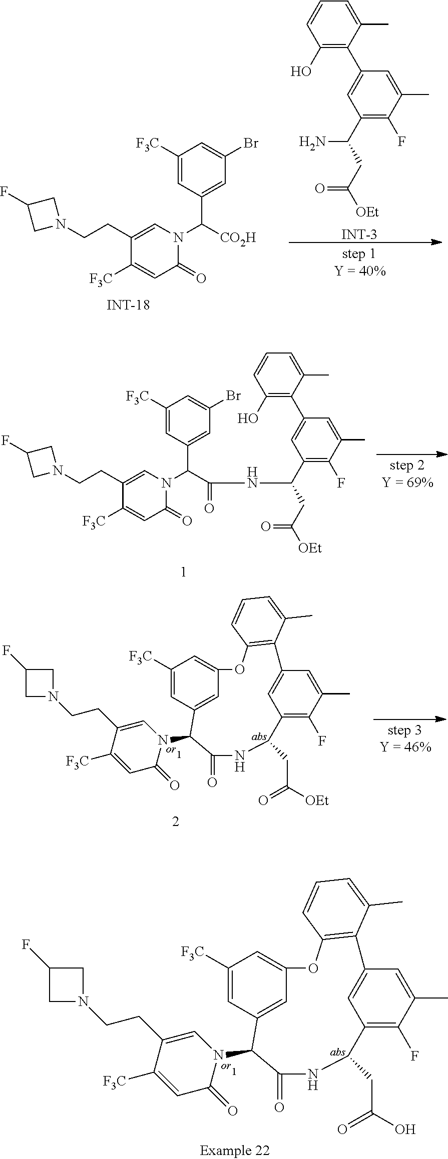

### Step 1: Preparation of ethyl (3S)-3-{2-[3-bromo-5-(trifluoromethyl)phenyl]-2-{5-[2-(3-fluoroazetidin-1-yl)ethyl]-2-oxo-4-(trifluoromethyl)pyridin-1-yl}acetamido}-3-{4-fluoro-2′-hydroxy-5,6′-dimethyl-[1,1′-biphenyl]-3-yl}propanoate

To a stirred solution of [3-bromo-5-(trifluoromethyl)phenyl]({5-[2-(3-fluoroazetidin-1-yl)ethyl]-2-oxo-4-(trifluoromethyl)pyridin-1-yl})acetic acid (160 mg, 0.29 mmol, 1.00 equiv.) and HOBT (52 mg, 0.38 mmol, 1.30 equiv.) in MeCN (1 mL) was added EDCI (84 mg, 0.44 mmol, 1.50 equiv.) at room temperature under nitrogen atmosphere. The resulting mixture was stirred for 1 h at 40° C. under nitrogen atmosphere. To the above mixture was added ethyl (3S)-3-amino-3-{4-fluoro-2′-hydroxy-5,6′-dimethyl-[1,1′-biphenyl]-3-yl}propanoate (97 mg, 0.29 mmol, 1.00 equiv.) and Et₃N (178 mg, 1.75 mmol, 6.00 equiv.) in MeCN (1 mL) at 0° C. The resulting mixture was stirred for an additional 2 days at 40° C. and then cooled to room temperature and concentrated. The residue was purified by reversed-phase flash chromatography (C18 gel; mobile phase, MeCN in water (0.1% NH₃·H₂O), 40% to 70% gradient in 20 min; detector, UV 220 nm) to afford ethyl (3S)-3-{2-[3-bromo-5-(trifluoromethyl)phenyl]-2-{5-[2-(3-fluoroazetidin-1-yl)ethyl]-2-oxo-4-(trifluoromethyl)pyridin-1-yl}acetamido}-3-{4-fluoro-2′-hydroxy-5,6′-dimethyl-[1,1′-biphenyl]-3-yl}propanoate (100 mg, 39.7%) as a red solid. LC-MS: (ES+H, m/z) [M+H]⁺=858.0.

### Step 2: Preparation of ethyl 2-[(14R*,17S)-19-fluoro-14-{5-[2-(3-fluoroazetidin-1-yl)ethyl]-2-oxo-4-(trifluoromethyl)pyridin-1-yl}-3,20-dimethyl-15-oxo-11-(trifluoromethyl)-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate

To a stirred solution of ethyl (3S)-3-{2-[3-bromo-5-(trifluoromethyl)phenyl]-2-{5-[2-(3-fluoroazetidin-1-yl)ethyl]-2-oxo-4-(trifluoromethyl)pyridin-1-yl}acetamido}-3-{4-fluoro-2′-hydroxy-5,6′-dimethyl-[1,1′-biphenyl]-3-yl}propanoate (80 mg, 0.09 mmol, 1.00 equiv.) and (1R,2R)—N1,N2-dimethylcyclohexane-1,2-diamine (16 mg, 0.11 mmol, 1.20 equiv.) in dioxane (1 mL) were added CuI (5 mg, 0.03 mmol, 0.30 equiv.) and K₂CO₃ (26 mg, 0.19 mmol, 2.00 equiv.) at room temperature under nitrogen atmosphere. The resulting mixture was stirred for 2 h at 100° C. under nitrogen atmosphere and then cooled to room temperature. The resulting mixture was filtered, and the filter cake was washed with EtOAc (3×5 mL). The filtrate was concentrated under reduced pressure and the residue was purified by reversed-phase flash chromatography (C18 gel; mobile phase, MeCN in water (0.1% NH₃·H₂O), 40% to 70% gradient in 20 min; detector, UV 220 nm) to afford ethyl 2-[(14R*,17S)-19-fluoro-14-{5-[2-(3-fluoroazetidin-1-yl)ethyl]-2-oxo-4-(trifluoromethyl)pyridin-1-yl}-3,20-dimethyl-15-oxo-11-(trifluoromethyl)-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (50 mg, 69.0%) as a yellow solid. LC-MS: (ES+H, m/z) [M+H]⁺=778.3.

### Step 3: Preparation of [(14R*,17S)-19-fluoro-14-{5-[2-(3-fluoroazetidin-1-yl)ethyl]-2-oxo-4-(trifluoromethyl)pyridin-1-yl}-3,20-dimethyl-15-oxo-11-(trifluoromethyl)-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid

A stirred solution of ethyl 2-[(14R*,17S)-19-fluoro-14-{5-[2-(3-fluoroazetidin-1-yl)ethyl]-2-oxo-4-(trifluoromethyl)pyridin-1-yl}-3,20-dimethyl-15-oxo-11-(trifluoromethyl)-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (40 mg, 0.05 mmol, 1.00 equiv.) in THF (1 mL) was added LiOH (0.3 mL, 0.600 mmol, 2 M in water) at room temperature under nitrogen atmosphere. The resulting mixture was stirred for 2 h at room temperature and then acidified to pH 4 with 3 M HCl (aq.) and concentrated. The crude product (100 mg) was purified by Prep-HPLC (Column: Xselect CSH C18 OBD Column 30*150 mm, 5 μm; Mobile Phase A: water (0.1% HCOOH), Mobile Phase B: 20 mm NaOH+10% MeCN; Flow rate: 60 mL/min; Gradient: 25% B to 43% B in 8 min; Wavelength: 254 nm/220 nm; RT1 (min) 9.85) to afford [(14R*,17S)-19-fluoro-14-{5-[2-(3-fluoroazetidin-1-yl)ethyl]-2-oxo-4-(trifluoromethyl)pyridin-1-yl}-3,20-dimethyl-15-oxo-11-(trifluoromethyl)-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid (19 mg, 46.3%) as a white solid. LC-MS: (ES+H, m/z) [M+H]⁺=750.1. ¹H NMR (300 MHz, DMSO-d6) δ 9.06 (d, J=7.1 Hz, 1H), 7.91-7.73 (m, 2H), 7.62 (s, 1H), 7.43 (t, J=7.9 Hz, 1H), 7.30-7.12 (m, 2H), 7.03 (d, J=7.5 Hz, 1H), 6.81 (s, 1H), 6.53 (s, 1H), 6.36 (d, J=6.1 Hz, 1H), 6.25 (s, 1H), 5.47 (d, J=7.4 Hz, 1H), 5.01-4.65 (m, 1H), 3.31-3.13 (m, 2H), 2.85 (dd, J=26.0, 6.4 Hz, 2H), 2.63 (d, J=10.8 Hz, 2H), 2.32 (d, J=12.2 Hz, 7H), 1.96 (s, 3H). ¹⁹F NMR (282 MHz, DMSO-d₆) δ −60.92, −62.59, −125.65, −177.11.

### Example 23

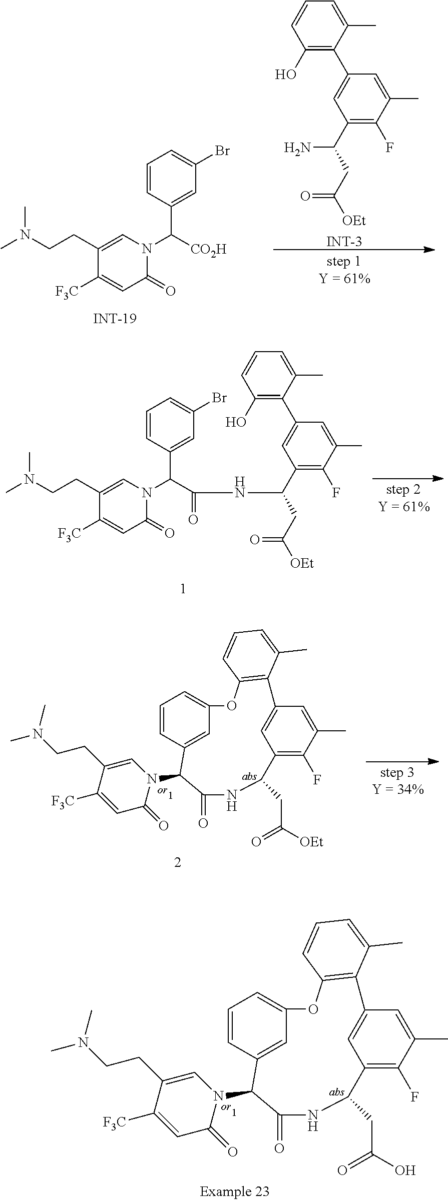

### Step 1: Preparation of ethyl (3S)-3-[2-(3-bromophenyl)-2-{5-[2-(dimethylamino)ethyl]-2-oxo-4-(trifluoromethyl)pyridin-1-yl}acetamido]-3-{4-fluoro-2′-hydroxy-5,6′-dimethyl-[1,1′-biphenyl]-3-yl}propanoate

To a stirred mixture of (3-bromophenyl)({5-[2-(dimethylamino)ethyl]-2-oxo-4-(trifluoromethyl)pyridin-1-yl})acetic acid (300 mg, 0.67 mmol, 1.00 equiv.) in MeCN (3 mL) were added EDCI (257 mg, 1.34 mmol, 2.00 equiv.) and HOBT (136 mg, 1.00 mmol, 1.50 equiv.) at room temperature under nitrogen atmosphere. The resulting mixture was stirred for 1 h at room temperature and then treated with a solution of ethyl (3S)-3-amino-3-{4-fluoro-2′-hydroxy-5,6′-dimethyl-[1,1′-biphenyl]-3-yl}propanoate (289 mg, 0.87 mmol, 1.30 equiv.) and Et₃N (407 mg, 4.02 mmol, 6.00 equiv.) in MeCN (3 mL) in portions over 3 min at room temperature. The resulting mixture was stirred for an additional 4 h at room temperature and then concentrated. The residue was purified by reversed-phase flash chromatography (C18 gel; mobile phase, MeCN in water (10 mmol/L NH₄HCO₃), 10% to 50% gradient in 10 min; detector, UV 254/220 nm) to afford ethyl (3S)-3-[2-(3-bromophenyl)-2-{5-[2-(dimethylamino)ethyl]-2-oxo-4-(trifluoromethyl)pyridin-1-yl}acetamido]-3-{4-fluoro-2′-hydroxy-5,6′-dimethyl-[1,1′-biphenyl]-3-yl}propanoate (310 mg, 60.7%) as a yellow oil. LC-MS: (ES+H, m/z) [M+H]⁺=760.1. ¹H NMR (400 MHz, DMSO-d₆) δ 9.29 (d, J=8.1 Hz, 1H), 9.09 (d, J=4.8 Hz, 1H), 7.67-7.39 (m, 3H), 7.38-7.29 (m, 1H), 7.22 (t, J=7.9 Hz, 1H), 7.14-6.98 (m, 3H), 6.91-6.80 (m, 1H), 6.75-6.65 (m, 3H), 5.61 (dd, J=28.5, 6.5 Hz, 1H), 4.07-3.95 (m, 2H), 2.83-2.70 (m, 2H), 2.47-2.40 (m, 2H), 2.25 (d, J=2.2 Hz, 3H), 2.08 (s, 2H), 1.97 (s, 6H), 1.89 (d, J=10.5 Hz, 3H), 1.12-1.06 (m, 3H). ¹⁹F NMR (377 MHz, DMSO-d₆) δ −62.77, 126.54.

### Step 2: Preparation of ethyl 2-[(14R*,17S)-14-{5-[2-(dimethylamino)ethyl]-2-oxo-4-(trifluoromethyl)pyridin-1-yl}-19-fluoro-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate

To a stirred mixture of ethyl (3S)-3-[2-(3-bromophenyl)-2-{5-[2-(dimethylamino)ethyl]-2-oxo-4-(trifluoromethyl)pyridin-1-yl}acetamido]-3-{4-fluoro-2′-hydroxy-5,6′-dimethyl-[1,1′-biphenyl]-3-yl}propanoate (310 mg, 0.40 mmol, 1.00 equiv.) in 1,4-dioxane (6 mL) were added CuI (39 mg, 0.20 mmol, 0.50 equiv.), K₂CO₃ (113 mg, 0.81 mmol, 2.00 equiv.) and (1R,2R)—N1,N2-dimethylcyclohexane-1,2-diamine (58 mg, 0.40 mmol, 1.00 equiv.) at room temperature under nitrogen atmosphere. The resulting mixture was stirred overnight at 100° C. and then cooled to room temperature and concentrated. The residue was purified by reversed-phase flash chromatography (C18 gel; mobile phase, MeCN in water (0.1% NH₃·H₂O), 10% to 50% gradient in 10 min; detector, UV 254/220 nm) to afford ethyl 2-[(14R*,17S)-14-{5-[2-(dimethylamino)ethyl]-2-oxo-4-(trifluoromethyl)pyridin-1-yl}-19-fluoro-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (170 mg, 61.3%) as a yellow oil. LC-MS: (ES+H, m/z) [M+H]⁺=680.2.

### Step 3: Preparation of [(14R*,17S)-14-{5-[2-(dimethylamino)ethyl]-2-oxo-4-(trifluoromethyl)pyridin-1-yl}-19-fluoro-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid

To a stirred mixture of ethyl 2-[(14R*,17S)-14-{5-[2-(dimethylamino)ethyl]-2-oxo-4-(trifluoromethyl)pyridin-1-yl}-19-fluoro-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (170 mg, 0.25 mmol, 1.00 equiv.) in THF (4 mL) was added LiOH·H₂O (21 mg, 0.50 mmol, 2.00 equiv.) in H₂O (1 mL) at room temperature under nitrogen atmosphere. After 2 h, the reaction was diluted with water (2 mL), acidified to pH 4 with 1 M HCl (aq.), and concentrated. The crude product (120 mg) was purified by Prep-HPLC (Column: XSelect CSH Prep C18 OBD Column, 30*150 mm, 5 μm; Mobile Phase A: water (0.1% HCOOH), Mobile Phase B: 20 mm NaOH+10% MeCN; Flow rate: 60 mL/min; Gradient: 14% B to 34% B in 10 min; Wavelength: 254/220 nm; RT1 (min) 9.87) to afford [(14R*,17S)-14-{5-[2-(dimethylamino)ethyl]-2-oxo-4-(trifluoromethyl)pyridin-1-yl}-19-fluoro-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid (55.6 mg, 33.9%, ee %=100%) as an off-white solid. LC-MS: (ES+H, m/z) [M+H]⁺=652.30. ¹H NMR (400 MHz, DMSO-d₆) δ 8.97 (d, J=7.3 Hz, 1H), 7.58 (d, J=8.1 Hz, 1H), 7.49 (dd, J=8.3, 2.3 Hz, 1H), 7.37 (td, J=7.9, 2.9 Hz, 2H), 7.28 (s, 1H), 7.18 (d, J=7.6 Hz, 1H), 7.07 (d, J=7.5 Hz, 1H), 7.00 (d, J=6.9, 2.1 Hz, 1H), 6.79 (s, 1H), 6.49 (s, 1H), 6.40 (d, J=6.3, 2.2 Hz, 1H), 6.00 (t, J=2.0 Hz, 1H), 5.52-5.44 (m, 1H), 2.73-2.65 (m, 1H), 2.64-2.57 (m, 1H), 2.46-2.39 (m, 2H), 2.29 (s, 3H), 2.27-2.19 (m, 1H), 2.15-2.09 (m, 1H), 1.95 (s, 3H), 1.91 (s, 6H). ¹⁹F NMR (377 MHz, DMSO-d₆) δ −62.70, −125.92.

### Example 24

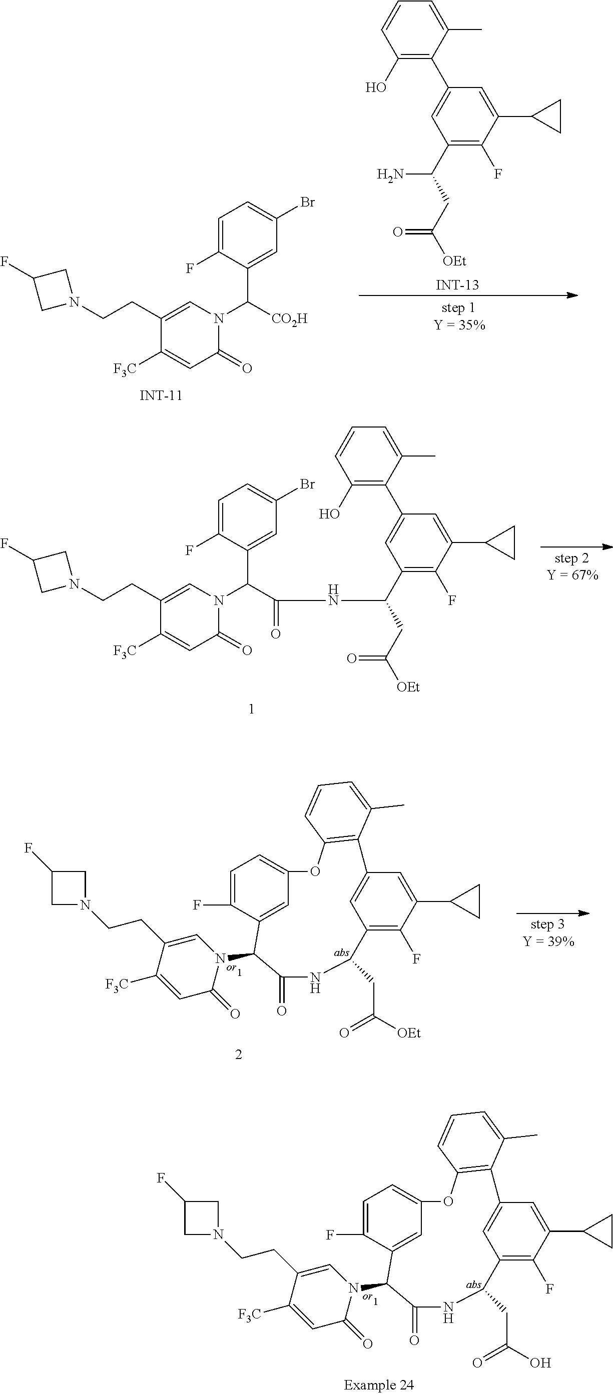

### Step 1: Preparation of ethyl (3S)-3-[2-(5-bromo-2-fluorophenyl)-2-{5-[2-(3-fluoroazetidin-1-yl)ethyl]-2-oxo-4-(trifluoromethyl)pyridin-1-yl}acetamido]-3-{5-cyclopropyl-4-fluoro-2′-hydroxy-6′-methyl-[1,1′-biphenyl]-3-yl}propanoate

A solution of (5-bromo-2-fluorophenyl)({5-[2-(3-fluoroazetidin-1-yl)ethyl]-2-oxo-4-(trifluoromethyl)pyridin-1-yl})acetic acid (400 mg, 0.81 mmol, 1.00 equiv.), HOBT (131 mg, 0.97 mmol, 1.20 equiv.) and EDCI (217 mg, 1.13 mmol, 1.40 equiv.) in CH₃CN (5 mL) was stirred for 1 h at room temperature under nitrogen atmosphere. To the above mixture was added ethyl (3S)-3-amino-3-{5-cyclopropyl-4-fluoro-2′-hydroxy-6′-methyl-[1,1′-biphenyl]-3-yl}propanoate (290 mg, 0.81 mmol, 1.00 equiv.) and Et₃N (821 mg, 8.11 mmol, 10.00 equiv.) in CH₃CN (5 mL) dropwise over 3 min at room temperature. The resulting mixture was stirred overnight at room temperature and then diluted with EtOAc (100 mL), washed with water (3×20 mL), brine (1×20 mL), filtered, and concentrated. The residue was purified by reversed-phase flash chromatography (C18 gel; mobile phase, MeCN in water (0.1% NH₃·H₂O), 0% to 80% gradient in 50 min; detector, UV 220 nm) to provide ethyl (3S)-3-[2-(5-bromo-2-fluorophenyl)-2-{5-[2-(3-fluoroazetidin-1-yl)ethyl]-2-oxo-4-(trifluoromethyl)pyridin-1-yl}acetamido]-3-{5-cyclopropyl-4-fluoro-2′-hydroxy-6′-methyl-[1,1′-biphenyl]-3-yl}propanoate (250 mg, 35.0%) as a yellow solid. LC-MS: (ES+H, m/z) [M+H]⁺=834.1.

### Step 2: Preparation of ethyl 2-[(14R*,17S)-20-cyclopropyl-12,19-difluoro-14-{5-[2-(3-fluoroazetidin-1-yl)ethyl]-2-oxo-4-(trifluoromethyl)pyridin-1-yl}-3-methyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate

To a stirred solution of ethyl (3S)-3-[2-(5-bromo-2-fluorophenyl)-2-{5-[2-(3-fluoroazetidin-1-yl)ethyl]-2-oxo-4-(trifluoromethyl)pyridin-1-yl}acetamido]-3-{5-cyclopropyl-4-fluoro-2′-hydroxy-6′-methyl-[1,1′-biphenyl]-3-yl}propanoate (250 mg, 0.30 mmol, 1.00 equiv.) and (1R,2R)—N1,N2-dimethylcyclohexane-1,2-diamine (55 mg, 0.39 mmol, 1.00 equiv.) in dioxane (12 mL) were added CuI (28 mg, 0.15 mmol, 0.50 equiv.) and K₂CO₃ (82 mg, 0.60 mmol, 2.00 equiv.) in portions at room temperature. The resulting mixture was stirred overnight at 100° C. under nitrogen atmosphere and then cooled to room temperature. The resulting mixture was diluted with EtOAc (200 mL) and washed with water (2×20 mL), brine (1×30 mL), filtered and concentrated. The residue was purified by reversed-phase flash chromatography (C18 gel; mobile phase, MeCN in water (0.1% NH₃·H₂O), 0% to 80% gradient in 50 min; detector, UV 220 nm) to provide ethyl 2-[(14R*,17S)-20-cyclopropyl-12,19-difluoro-14-{5-[2-(3-fluoroazetidin-1-yl)ethyl]-2-oxo-4-(trifluoromethyl)pyridin-1-yl}-3-methyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (160 mg, 67.3%) as a as a yellow solid. LC-MS: (ES+H, m/z) [M+H]⁺=754.2. ¹H NMR (400 MHz, CD₃OD) δ7.57-7.53 (m, 1H), 7.45 (d, J=8.0 Hz, 1H), 7.35 (dd, J=16.0, 8.3 Hz, 2H), 7.20-7.14 (m, 2H), 6.83 (d, J=22.3 Hz, 2H), 6.66 (dd, J=6.9, 2.2 Hz, 1H), 6.40 (dd, J=6.3, 2.1 Hz, 1H), 6.09 (dd, J=6.1, 2.9 Hz, 1H), 5.72 (t, J=7.9 Hz, 1H), 4.58 (s, 1H), 4.18-4.06 (m, 2H), 3.47-3.35 (m, 2H), 3.06-2.96 (m, 2H), 2.89-2.71 (m, 2H), 2.58-2.38 (m, 4H), 2.21-2.14 (m, 1H), 2.03 (s, 1H), 1.97 (s, 3H), 1.19 (t, J=7.1 Hz, 3H), 1.05-1.01 (m, 2H), 0.82-0.66 (m, 2H).

### Step 3: Preparation of [(14R*,17S)-20-cyclopropyl-12,19-difluoro-14-{5-[2-(3-fluoroazetidin-1-yl)ethyl]-2-oxo-4-(trifluoromethyl)pyridin-1-yl}-3-methyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid

To a stirred mixture of ethyl 2-[(14R*,17S)-20-cyclopropyl-12,19-difluoro-14-{5-[2-(3-fluoroazetidin-1-yl)ethyl]-2-oxo-4-(trifluoromethyl)pyridin-1-yl}-3-methyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (160 mg, 0.21 mmol, 1.00 equiv.) in THF (4 mL) was added LiOH·H₂O (44 mg, 1.06 mmol, 5.00 equiv.) in H₂O (1 mL) dropwise at room temperature. After 3 h the reaction was neutralized to pH 7 with HCl (aq. 1 M) and concentrated. The crude product (200 mg) was purified by Prep-HPLC (Column: XBridge Shield RP18 OBD Column, 50*250 mm, 10 μm; Mobile Phase A: water (0.05% HCOOH), Mobile Phase B: CH₃CN; Flow rate: 100 mL/min; Gradient: 20% B to 50% B in 30 min, 30% B; Wavelength: 254/220 nm; RT1 (min) 19.2) to afford [(14R*,17S)-20-cyclopropyl-12,19-difluoro-14-{5-[2-(3-fluoroazetidin-1-yl)ethyl]-2-oxo-4-(trifluoromethyl)pyridin-1-yl}-3-methyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid (60.8 mg, 38.6%) as a white solid. LC-MS: (ES+H, m/z) [M+H]⁺=726.25. ¹H NMR (300 MHz, CD₃OD) δ 7.58-7.55 (m, 1H), 7.47 (d, J=8.1 Hz, 1H), 7.39-7.33 (m, 2H), 7.21-7.15 (m, 2H), 6.90 (d, J=1.3 Hz, 2H), 6.67 (dd, J=6.9, 2.1 Hz, 1H), 6.45 (dd, J=6.3, 2.1 Hz, 1H), 6.14 (dd, J=6.2, 2.9 Hz, 1H), 5.75 (t, J=7.6 Hz, 1H), 5.17-4.95 (m, 1H), 3.71 (dd, J=18.8, 11.3 Hz, 2H), 3.39 (dd, J=9.3, 4.7 Hz, 1H), 3.29 (d, J=4.4 Hz, 1H), 2.79-2.72 (m, 4H), 2.54-2.51 (m, 2H), 2.20-1.99 (m, 1H), 1.99 (s, 3H), 1.06-1.02 (m, 2H), 0.80-0.71 (m, 2H). ¹⁹F NMR (377 MHz, CD₃OD) δ −64.99, −123.20, −129.94, −180.47.

### Example 25

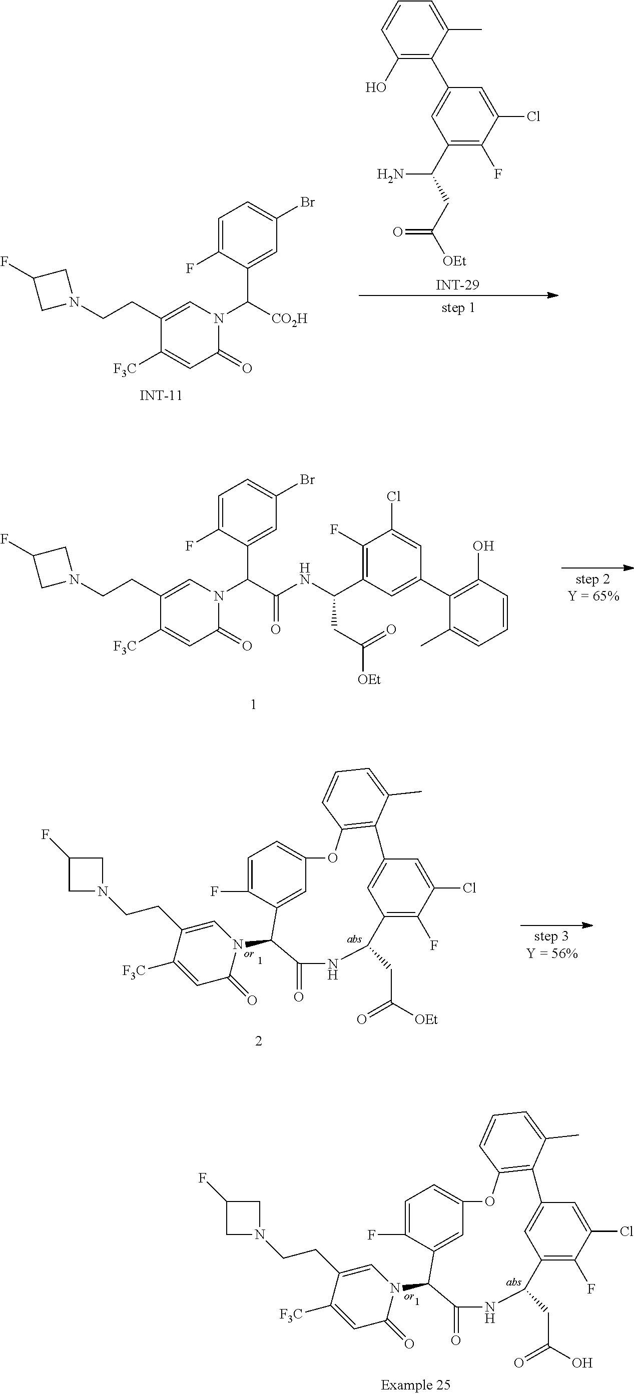

### Step 1: Preparation of ethyl (3S)-3-[2-(5-bromo-2-fluorophenyl)-2-{5-[2-(3-fluoroazetidin-1-yl)ethyl]-2-oxo-4-(trifluoromethyl)pyridin-1-yl}acetamido]-3-{5-chloro-4-fluoro-2′-hydroxy-6′-methyl-[1,1′-biphenyl]-3-yl}propanoate

To a stirred solution of (5-bromo-2-fluorophenyl)({5-[2-(3-fluoroazetidin-1-yl)ethyl]-2-oxo-4-(trifluoromethyl)pyridin-1-yl})acetic acid (422 mg, 0.85 mmol, 1.00 equiv.) in acetonitrile (5 mL) were added EDCI·HCl (198 mg, 1.28 mmol, 1.50 equiv.) and HOBT (172 mg, 1.28 mmol, 1.50 equiv.) in portions at room temperature under nitrogen atmosphere. After 1 h, ethyl (3S)-3-amino-3-{5-chloro-4-fluoro-2′-hydroxy-6′-methyl-[1,1′-biphenyl]-3-yl}propanoate (300 mg, crude) and Et₃N (431 mg, 4.26 mmol, 5.00 equiv.) in acetonitrile (3 mL) were added dropwise over 5 min at room temperature. After 2 h the reaction was concentrated and the residue was purified by reversed-phase flash chromatography (C18; mobile phase, MeCN in water (0.1% NH₃·H₂O), 10% to 50% gradient in 25 min; detector, UV 220 nm) to afford ethyl (3S)-3-[2-(5-bromo-2-fluorophenyl)-2-{5-[2-(3-fluoroazetidin-1-yl)ethyl]-2-oxo-4-(trifluoromethyl)pyridin-1-yl}acetamido]-3-{5-chloro-4-fluoro-2′-hydroxy-6′-methyl-[1,1′-biphenyl]-3-yl}propanoate (256 mg, 38.7%, over 2 steps) as a colorless oil. LC-MS: (ES+H, m/z) [M+H]⁺=828.10.

### Step 2: Preparation of ethyl 2-[(14R*,17S)-20-chloro-12,19-difluoro-14-{5-[2-(3-fluoroazetidin-1-yl)ethyl]-2-oxo-4-(trifluoromethyl)pyridin-1-yl}-3-methyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate

To a stirred solution of ethyl (3S)-3-[2-(5-bromo-2-fluorophenyl)-2-{5-[2-(3-fluoroazetidin-1-yl)ethyl]-2-oxo-4-(trifluoromethyl)pyridin-1-yl}acetamido]-3-{5-chloro-4-fluoro-2′-hydroxy-6′-methyl-[1,1′-biphenyl]-3-yl}propanoate (240 mg, 0.28 mmol, 1.00 equiv.) and (1R,2R)—N1,N2-dimethylcyclohexane-1,2-diamine (39 mg, 0.27 mmol, 1.00 equiv.) in 1,4-dioxane (6 mL) were added K₂CO₃ (76 mg, 0.55 mmol, 2.00 equiv.) and CuI (26 mg, 0.13 mmol, 0.50 equiv.) in portions at room temperature under nitrogen atmosphere. The resulting mixture was stirred overnight at 100° C. under nitrogen atmosphere and then cooled to room temperature and concentrated. The residue was purified by Prep-TLC (CH₂Cl₂/MeOH, 20:1) to afford ethyl 2-[(14R*,17S)-20-chloro-12,19-difluoro-14-{5-[2-(3-fluoroazetidin-1-yl)ethyl]-2-oxo-4-(trifluoromethyl)pyridin-1-yl}-3-methyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (135 mg, 64.9%) as a white solid. LC-MS: (ES+H, m/z) [M+H]⁺=748.20.

### Step 3: Preparation of [(14R*,17S)-20-chloro-12,19-difluoro-14-{5-[2-(3-fluoroazetidin-1-yl)ethyl]-2-oxo-4-(trifluoromethyl)pyridin-1-yl}-3-methyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid

To a stirred solution of ethyl 2-[(14R*,17S)-20-chloro-12,19-difluoro-14-{5-[2-(3-fluoroazetidin-1-yl)ethyl]-2-oxo-4-(trifluoromethyl)pyridin-1-yl}-3-methyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (120 mg, 0.16 mmol, 1.00 equiv.) in THF (5 mL) was added LiOH·H₂O (13 mg, 0.32 mmol, 2.00 equiv.) in water (2.5 mL dropwise at 0° C. under nitrogen atmosphere. The resulting mixture was stirred for 2 h at 30° C. and then diluted with THF (10 mL), acidified to pH 5˜6 with 1 M HCl (aq.), and concentrated. The crude product (100 mg) was purified by Prep-HPLC (Column: XSelect CSH Prep C18 OBD Column, 30*150 mm, 5 μm; Mobile Phase A: water (0.1% HCOOH), Mobile Phase B: MeCN; Flow rate: 60 mL/min; Gradient: 18% B to 38% B in 10 min; Wavelength: 254 nm/220 nm; RT1 (min) 9.87) to afford [(14R*,17S)-20-chloro-12,19-difluoro-14-{5-[2-(3-fluoroazetidin-1-yl)ethyl]-2-oxo-4-(trifluoromethyl)pyridin-1-yl}-3-methyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid (64.8 mg, 56.1%) as a white solid. LC-MS: (ES+H, m/z) [M+H]⁺=720.10. ¹H NMR (400 MHz, DMSO-d₆) δ 9.22 (d, J=7.0 Hz, 1H), 7.73-7.59 (m, 2H), 7.50-7.36 (m, 2H), 7.29 (t, J=9.1 Hz, 1H), 7.25 (s, 1H), 7.22 (d, J=7.6 Hz, 1H), 6.81 (s, 1H), 6.68 (s, 1H), 6.60 (dd, J=6.0, 2.1 Hz, 1H), 5.89 (dd, J=6.2, 3.0 Hz, 1H), 5.43 (q, J=7.4 Hz, 1H), 4.86 (m, 1H), 3.29-3.23 (m, 2H), 2.92-2.86 (m, 1H), 2.85-2.81 (m, 1H), 2.80-2.69 (m, 2H), 2.45-2.26 (m, 4H), 1.97 (s, 3H). ¹⁹F NMR (400 MHz, DMSO-d₆) δ −62.621, −121.456, −122.736, −176.961.

### Example 26

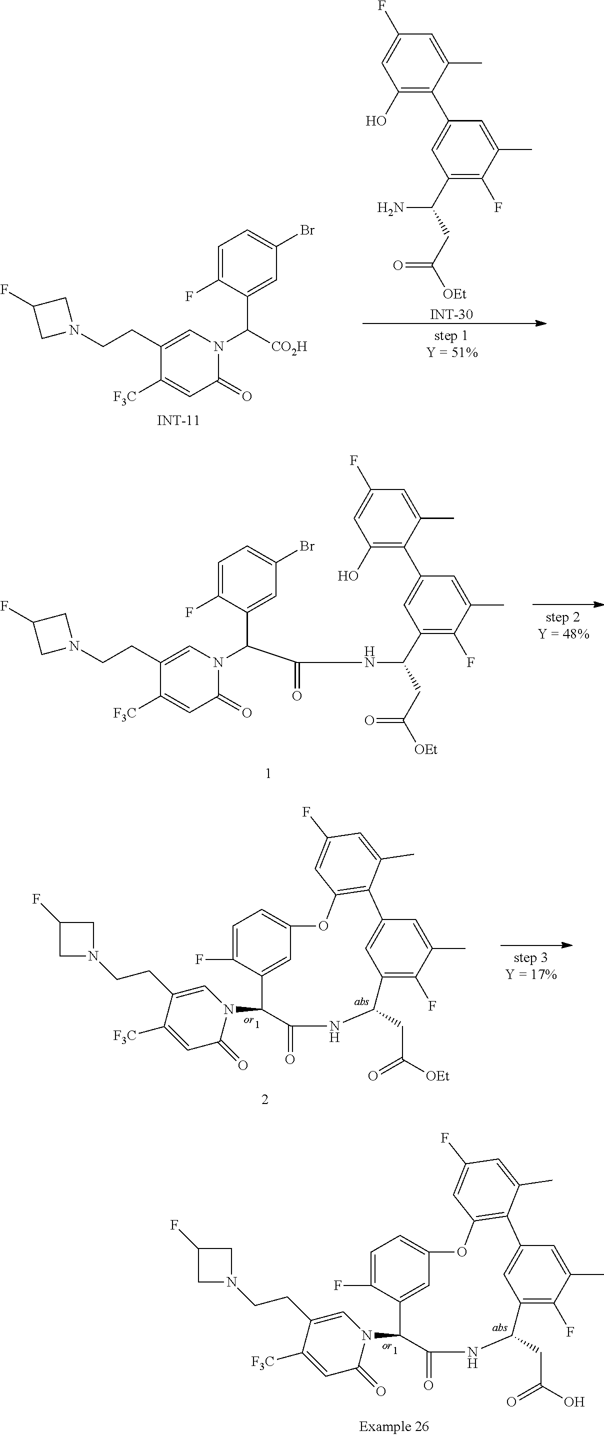

### Step 1: Preparation of ethyl (3S)-3-[2-(5-bromo-2-fluorophenyl)-2-{5-[2-(3-fluoroazetidin-1-yl)ethyl]-2-oxo-4-(trifluoromethyl)pyridin-1-yl}acetamido]-3-{4,4′-difluoro-2′-hydroxy-5,6′-dimethyl-[1,1′-biphenyl]-3-yl}propanoate

A solution of ethyl (3S)-3-amino-3-{4,4′-difluoro-2′-hydroxy-5,6′-dimethyl-[1,1′-biphenyl]-3-yl}propanoate hydrochloride (211 mg, 0.60 mmol, 1.00 equiv.) and triethylamine (612 mg, 6.06 mmol, 10.00 equiv.) in MeCN (5 mL) was stirred for 1 h at 40° C. under nitrogen atmosphere. The resulting mixture was concentrated under reduced pressure. In a separate vial, a stirred solution of (5-bromo-2-fluorophenyl)({5-[2-(3-fluoroazetidin-1-yl)ethyl]-2-oxo-4-(trifluoromethyl)pyridin-1-yl})acetic acid (300 mg, 0.60 mmol, 1.00 equiv.) in MeCN (4 mL) was treated with HOBT (106 mg, 0.78 mmol, 1.30 equiv.) and EDCI (174 mg, 0.90 mmol, 1.50 equiv.) at room temperature under nitrogen atmosphere. And then heated to 40° C. for 1 h. To the above mixture was added a solution of ethyl (3S)-3-amino-3-{4,4′-difluoro-2′-hydroxy-5,6′-dimethyl-[1,1′-biphenyl]-3-yl}propanoate in MeCN (2 mL) dropwise at 0° C. The resulting mixture was stirred overnight at 40° C. and then concentrated. The residue was purified by reversed-phase flash chromatography (C18 gel; mobile phase, MeCN in water (0.1% NH₃·H₂O), 30% to 80% gradient in 30 min; detector, UV 220 nm) to provide ethyl (3S)-3-[2-(5-bromo-2-fluorophenyl)-2-{5-[2-(3-fluoroazetidin-1-yl)ethyl]-2-oxo-4-(trifluoromethyl)pyridin-1-yl}acetamido]-3-{4,4′-difluoro-2′-hydroxy-5,6′-dimethyl-[1,1′-biphenyl]-3-yl}propanoate (253 mg, 50.5%) as a white solid. LC-MS: (ES+H, m/z) [M+H]⁺=826.2.

### Step 2: Preparation of ethyl 2-[(14R*,17S)-5,12,19-trifluoro-14-{5-[2-(3-fluoroazetidin-1-yl)ethyl]-2-oxo-4-(trifluoromethyl)pyridin-1-yl}-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate

To a stirred mixture of ethyl (3S)-3-[2-(5-bromo-2-fluorophenyl)-2-{5-[2-(3-fluoroazetidin-1-yl)ethyl]-2-oxo-4-(trifluoromethyl)pyridin-1-yl}acetamido]-3-{4,4′-difluoro-2′-hydroxy-5,6′-dimethyl-[1,1′-biphenyl]-3-yl}propanoate (253 mg, 0.30 mmol, 1.00 equiv.) and (1R,2R)—N1,N2-dimethylcyclohexane-1,2-diamine (52 mg, 0.36 mmol, 1.20 equiv.) in dioxane (10 mL) were added CuI (17 mg, 0.09 mmol, 0.30 equiv.) and K₂CO₃ (84 mg, 0.61 mmol, 2.00 equiv.) at room temperature under nitrogen atmosphere. The resulting mixture was stirred overnight at 100° C., cooled to room temperature and filtered. The filter cake was washed with EtOAc (3×100 mL) and the filtrate was concentrated under reduced pressure. The residue was purified by reversed-phase flash chromatography (C18 gel; mobile phase, MeCN in water (0.1% NH₃·H₂O), 30% to 60% gradient in 30 min; detector, UV 220 nm) to provide ethyl 2-[(14R*,17S)-5,12,19-trifluoro-14-{5-[2-(3-fluoroazetidin-1-yl)ethyl]-2-oxo-4-(trifluoromethyl)pyridin-1-yl}-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (110 mg, 48.2%) as a red solid. LC-MS: (ES+H, m/z) [M+H]⁺=746.2.

### Step 3: Preparation of [(14R*,17S)-5,12,19-trifluoro-14-{5-[2-(3-fluoroazetidin-1-yl)ethyl]-2-oxo-4-(trifluoromethyl)pyridin-1-yl}-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid

To a stirred mixture of ethyl 2-[(14R*,17S)-5,12,19-trifluoro-14-{5-[2-(3-fluoroazetidin-1-yl)ethyl]-2-oxo-4-(trifluoromethyl)pyridin-1-yl}-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (100 mg, 0.13 mmol, 1.00 equiv.) and H₂O (1 mL) in THF (3 mL) was added LiOH·H₂O (12 mg, 0.26 mmol, 2.00 equiv.) at room temperature under nitrogen atmosphere. The resulting mixture was stirred for 1 h at room temperature and then acidified to pH 5 with 1 M HCl (aq.), and concentrated. The crude product was purified by Prep-HPLC (Column: XSelect CSH Prep C18 OBD Column, 30*150 mm, 5 μm; Mobile Phase A: water (0.1% HCOOH), Mobile Phase B: MeCN; Flow rate: 60 mL/min; Gradient: 19% B to 39% B in 10 min; Wavelength: 254 nm/220 nm; RT1 (min) 9.68) to afford [(14R*,17S)-5,12,19-trifluoro-14-{5-[2-(3-fluoroazetidin-1-yl)ethyl]-2-oxo-4-(trifluoromethyl)pyridin-1-yl}-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid (16.5 mg, 16.9%) as a white solid. LC-MS: (ES+H, m/z) [M+H]⁺=718.15. ¹H NMR (400 MHz, DMSO-d₆) δ 9.19 (d, J=7.3 Hz, 1H), 7.77-7.69 (m, 1H), 7.66 (dd, J=9.7, 2.7 Hz, 1H), 7.30 (t, J=9.1 Hz, 1H), 7.21 (s, 1H), 7.12 (dd, J=9.6, 2.6 Hz, 1H), 7.03 (dd, J=7.1, 2.2 Hz, 1H), 6.80 (s, 1H), 6.69 (s, 1H), 6.45 (dd, J=6.4, 2.2 Hz, 1H), 5.96 (dd, J=6.2, 3.0 Hz, 1H), 5.52-5.42 (m, 1H), 4.80-4.95 (m, 1H), 3.34-3.21 (m, 2H), 2.92-2.77 (m, 2H), 2.74-2.57 (m, 2H), 2.44-2.36 (m, 1H), 2.36-2.27 (m, 6H), 1.96 (s, 3H). ¹⁹F NMR (377 MHz, DMSO-d₆) δ −61.94, −112.66, −121.19, −125.47, −176.97.

### Example 27

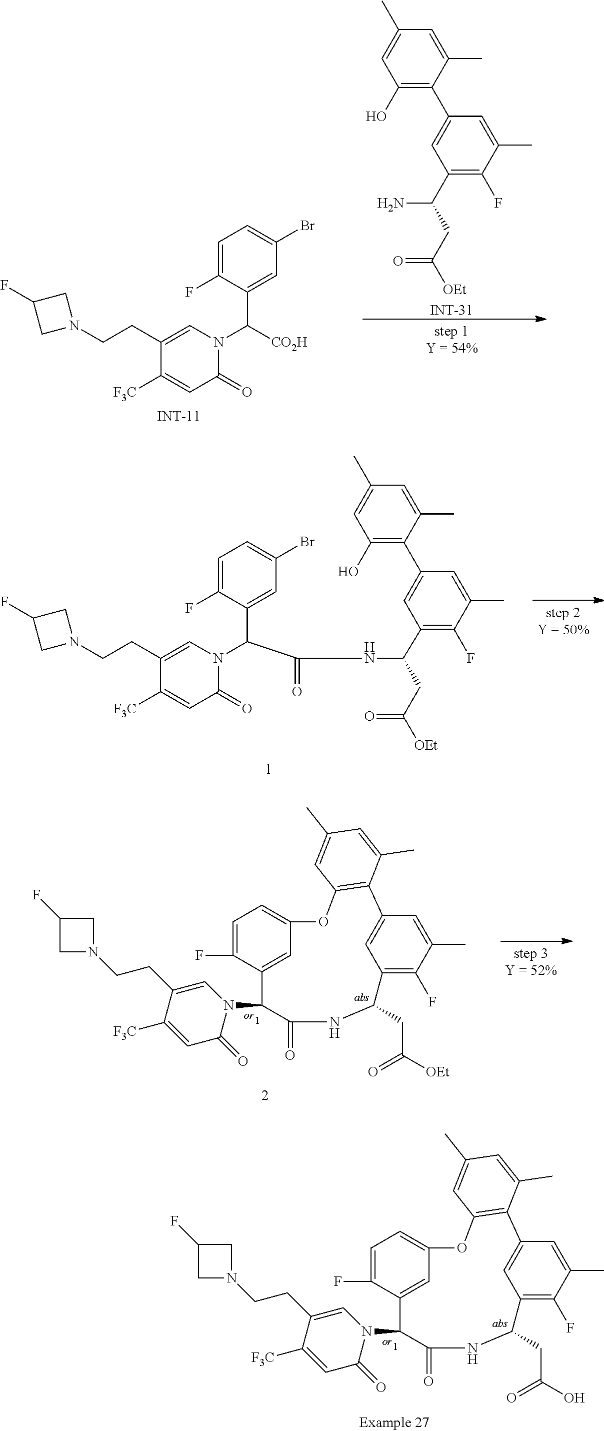

### Step 1: Preparation of ethyl (3S)-3-[2-(5-bromo-2-fluorophenyl)-2-{5-[2-(3-fluoroazetidin-1-yl)ethyl]-2-oxo-4-(trifluoromethyl)pyridin-1-yl}acetamido]-3-{4-fluoro-2′-hydroxy-4′,5,6′-trimethyl-[1,1′-biphenyl]-3-yl}propanoate

A mixture of ethyl (3S)-3-amino-3-{4-fluoro-2′-hydroxy-4′,5,6′-trimethyl-[1,1′-biphenyl]-3-yl}propanoate hydrochloride (260 mg, assumed 100% yield, 0.68 mmol, 1.00 equiv.) and NEt₃ (0.6 mL, 4.09 mmol, 6.00 equiv.) in MeCN (5 mL) was stirred for 30 min at 30° C. under nitrogen atmosphere and then concentrated. In a separate vial, a stirred mixture of (5-bromo-2-fluorophenyl)({5-[2-(3-fluoroazetidin-1-yl)ethyl]-2-oxo-4-(trifluoromethyl)pyridin-1-yl})acetic acid (337 mg, 0.68 mmol, 1.00 equiv.) and HOBT (120 mg, 0.89 mmol, 1.30 equiv.) in MeCN (5 mL) was added EDCI (196 mg, 1.02 mmol, 1.50 equiv.) at room temperature under nitrogen atmosphere and then stirred for 1 h at 30° C. under nitrogen atmosphere. To the above mixture was added the solution of ethyl (3S)-3-amino-3-{4-fluoro-2′-hydroxy-4′,5,6′-trimethyl-[1,1′-biphenyl]-3-yl}propanoate. The resulting mixture was stirred overnight at 30° C., concentrated and then purified by reversed-phase flash chromatography (C18 gel; mobile phase, MeCN in water (0.1% NH₃·H₂O), 40% to 60% gradient in 20 min; detector, UV 220 nm) to provide ethyl (3S)-3-[2-(5-bromo-2-fluorophenyl)-2-{5-[2-(3-fluoroazetidin-1-yl)ethyl]-2-oxo-4-(trifluoromethyl)pyridin-1-yl}acetamido]-3-{4-fluoro-2′-hydroxy-4′,5,6′-trimethyl-[1,1′-biphenyl]-3-yl}propanoate (280 mg, 54.2%, over two steps) as a white solid. LC-MS: (ES+H, m/z) [M+H]⁺=822.2.

### Step 2: Preparation of ethyl 2-[(14S,17S)-12,19-difluoro-14-{5-[2-(3-fluoroazetidin-1-yl)ethyl]-2-oxo-4-(trifluoromethyl)pyridin-1-yl}-3,5,20-trimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate

To a stirred mixture of ethyl (3S)-3-[2-(5-bromo-2-fluorophenyl)-2-{5-[2-(3-fluoroazetidin-1-yl)ethyl]-2-oxo-4-(trifluoromethyl)pyridin-1-yl}acetamido]-3-{4-fluoro-2′-hydroxy-4′,5,6′-trimethyl-[1,1′-biphenyl]-3-yl}propanoate (270 mg, 0.33 mmol, 1.00 equiv.) and (1R,2R)—N1,N2-dimethylcyclohexane-1,2-diamine (56 mg, 0.39 mmol, 1.20 equiv.) in dioxane (5 mL) were added CuI (19 mg, 0.098 mmol, 0.30 equiv.) and K₂CO₃ (91 mg, 0.66 mmol, 2.00 equiv.) at room temperature under nitrogen atmosphere. The resulting mixture was stirred overnight at 100° C., cooled to room temperature, and then filtered. The filter cake was washed with MeCN (3×3 mL) and the filtrate was concentrated under reduced pressure. The residue was purified by reversed-phase flash chromatography (C18 gel; mobile phase, MeCN in water (0.1% NH₃·H₂O), 40% to 60% gradient in 10 min; detector, UV 220 nm) to provide ethyl 2-[(14S,17S)-12,19-difluoro-14-{5-[2-(3-fluoroazetidin-1-yl)ethyl]-2-oxo-4-(trifluoromethyl)pyridin-1-yl}-3,5,20-trimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (120 mg, 49.3%) as a white solid. LC-MS: (ES+H, m/z) [M+H]⁺=742.3.

### Step 3: Preparation of [(14R*,17S)-12,19-difluoro-14-{5-[2-(3-fluoroazetidin-1-yl)ethyl]-2-oxo-4-(trifluoromethyl)pyridin-1-yl}-3,5,20-trimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid

A mixture of ethyl 2-[(14R*,17S)-12,19-difluoro-14-{5-[2-(3-fluoroazetidin-1-yl)ethyl]-2-oxo-4-(trifluoromethyl)pyridin-1-yl}-3,5,20-trimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (110 mg, 0.15 mmol, 1.00 equiv.) and LiOH·H₂O (11 mg, 0.44 mmol, 3.00 equiv.) in THF (3 mL) and H₂O (1 mL) was stirred for 2 h at room temperature under nitrogen atmosphere and then acidified to pH 5 with HCl (aq. 1 M) and concentrated. The resulting mixture was concentrated under reduced pressure. The residue was purified by Prep-HPLC: (Column: Xselect CSH C18 OBD Column 30*150 mm, 5 μm; Mobile Phase A: water (0.1% HCOOH), Mobile Phase B: ACN; Flow rate: 60 mL/min; Gradient: 25% B to 39% B in 8 min; Wavelength: 254 nm/220 nm; RT1 (min) 8.97) to afford [(14R*,17S)-12,19-difluoro-14-{5-[2-(3-fluoroazetidin-1-yl)ethyl]-2-oxo-4-(trifluoromethyl)pyridin-1-yl}-3,5,20-trimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid (55.1 mg, 52.1%) as a white solid. LC-MS: (ES+H, m/z) [M+H]⁺=714.15. ¹H NMR (400 MHz, CD₃OD) δ 7.55 (ddd, J=9.1, 4.1, 2.9 Hz, 1H), 7.35 (s, 1H), 7.26 (s, 1H), 7.16 (t, J=9.0 Hz, 1H), 6.98 (s, 1H), 6.94 (dd, J=7.0, 2.1 Hz, 1H), 6.86 (d, J=4.1 Hz, 2H), 6.45 (dd, J=6.3, 2.2 Hz, 1H), 6.10 (dd, J=6.1, 2.9 Hz, 1H), 5.68 (t, J=7.5 Hz, 1H), 5.09-4.89 (m, 1H), 3.69-3.53 (m, 2H), 3.29-3.18 (m, 2H), 2.84-2.60 (m, 4H), 2.48 (q, J=7.0 Hz, 2H), 2.40 (s, 3H), 2.33 (s, 3H), 1.95 (s, 3H). ¹⁹F NMR (377 MHz, CD₃OD) δ −65.00, −123.31, −126.99, −180.48.

### Example 28

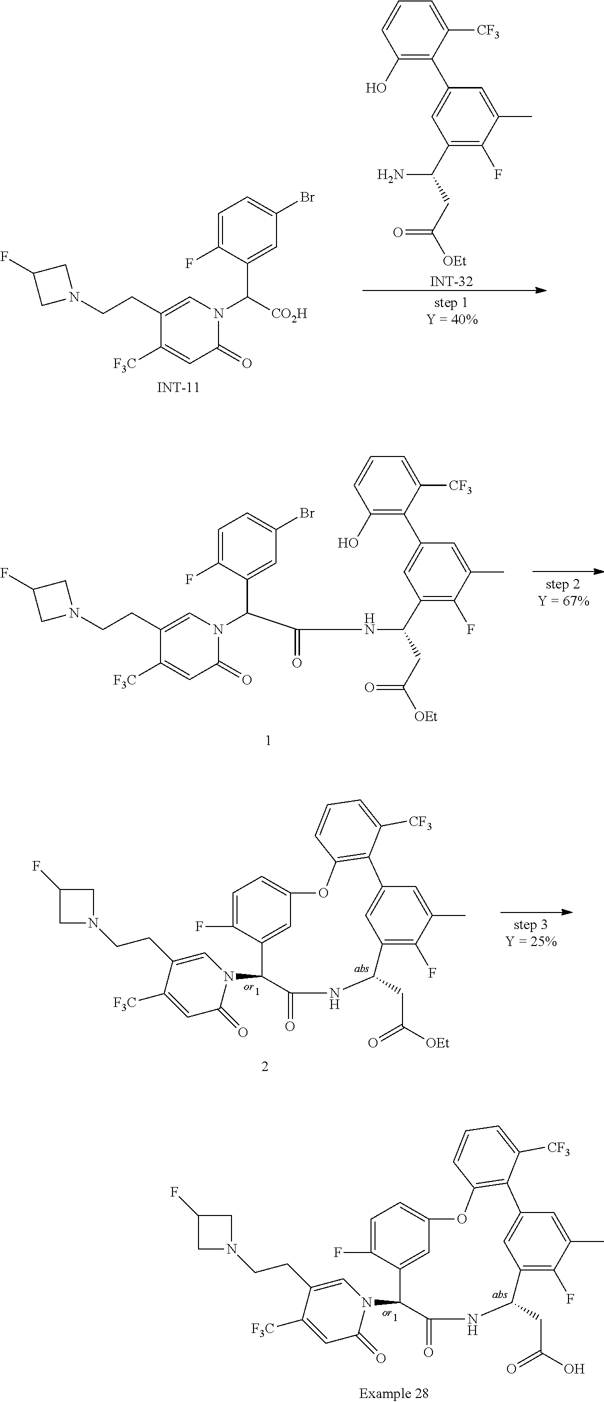

### Step 1: Preparation of ethyl (3S)-3-[2-(5-bromo-2-fluorophenyl)-2-{5-[2-(3-fluoroazetidin-1-yl)ethyl]-2-oxo-4-(trifluoromethyl)pyridin-1-yl}acetamido]-3-[4-fluoro-2′-hydroxy-5-methyl-6′-(trifluoromethyl)-[1,1′-biphenyl]-3-yl]propanoate

To a stirred mixture of (5-bromo-2-fluorophenyl)({5-[2-(3-fluoroazetidin-1-yl)ethyl]-2-oxo-4-(trifluoromethyl)pyridin-1-yl})acetic acid (300 mg, 0.61 mmol, 1.00 equiv.) in CH₃CN (6 mL) were added HOBT (106 mg, 0.79 mmol, 1.30 equiv.) and EDCI (174 mg, 0.91 mmol, 1.50 equiv.) in portions at room temperature under nitrogen atmosphere. The resulting mixture was stirred for 1 h at room temperature under nitrogen atmosphere and then treated with a solution of ethyl (3S)-3-amino-3-[4-fluoro-2′-hydroxy-5-methyl-6′-(trifluoromethyl)-[1,1′-biphenyl]-3-yl]propanoate hydrochloride (233 mg, 0.61 mmol, 1.00 equiv.) and NEt₃ (184 mg, 1.81 mmol, 3.00 equiv.) in CH₃CN (6 mL) dropwise over 5 min at 0° C. The resulting mixture was stirred overnight at room temperature and then concentrated and purified by silica gel column chromatography (CH₂Cl₂/MeOH (0% to 5% gradient in 20 min) to afford ethyl (3S)-3-[2-(5-bromo-2-fluorophenyl)-2-{5-[2-(3-fluoroazetidin-1-yl)ethyl]-2-oxo-4-(trifluoromethyl)pyridin-1-yl}acetamido]-3-[4-fluoro-2′-hydroxy-5-methyl-6′-(trifluoromethyl)-[1,1′-biphenyl]-3-yl]propanoate (210 mg, 40.2%) as a yellow solid. LC-MS: (ES+H, m/z) [M+H]⁺=862.2.

### Step 2: Preparation of ethyl 2-[(14R*,17S)-12,19-difluoro-14-{5-[2-(3-fluoroazetidin-1-yl)ethyl]-2-oxo-4-(trifluoromethyl)pyridin-1-yl}-20-methyl-15-oxo-3-(trifluoromethyl)-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate

To a stirred solution of ethyl (3S)-3-[2-(5-bromo-2-fluorophenyl)-2-{5-[2-(3-fluoroazetidin-1-yl)ethyl]-2-oxo-4-(trifluoromethyl)pyridin-1-yl}acetamido]-3-[4-fluoro-2′-hydroxy-5-methyl-6′-(trifluoromethyl)-[1,1′-biphenyl]-3-yl]propanoate (215 mg, 0.25 mmol, 1.00 equiv.) in dioxane (15 mL) were added CuI (24 mg, 0.12 mmol, 0.50 equiv.), K₂CO₃ (68 mg, 0.49 mmol, 2.00 equiv.) and N1,N2-dimethylcyclohexane-1,2-diamine (35 mg, 0.25 mmol, 1.00 equiv.) in portions at room temperature under nitrogen atmosphere. The resulting mixture was stirred overnight at 100° C. and then cooled to room temperature and concentrated. The residue was purified by silica gel column chromatography (CH₂Cl₂/MeOH (0% to 5% gradient in 20 min) to afford ethyl 2-[(14R*,17S)-12,19-difluoro-14-{5-[2-(3-fluoroazetidin-1-yl)ethyl]-2-oxo-4-(trifluoromethyl)pyridin-1-yl}-20-methyl-15-oxo-3-(trifluoromethyl)-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (130 mg, 66.7%) as a yellow solid. LC-MS: (ES+H, m/z) [M+H]⁺=782.2.

### Step 3: Preparation of [(14R*,17S)-12,19-difluoro-14-{5-[2-(3-fluoroazetidin-1-yl)ethyl]-2-oxo-4-(trifluoromethyl)pyridin-1-yl}-20-methyl-15-oxo-3-(trifluoromethyl)-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid

To a stirred solution of ethyl 2-[(14R*,17S)-12,19-difluoro-14-{5-[2-(3-fluoroazetidin-1-yl)ethyl]-2-oxo-4-(trifluoromethyl)pyridin-1-yl}-20-methyl-15-oxo-3-(trifluoromethyl)-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (130 mg, 0.16 mmol, 1.00 equiv.) in THF (2 mL) was added LiOH·H₂O (34 mg, 0.83 mmol, 5.00 equiv.) in H₂O (0.5 mL) dropwise at room temperature under nitrogen atmosphere. The resulting mixture was stirred for 3 h at room temperature, and then neutralized to pH 7 with HCl (2 M, aq.) and concentrated. The residue was purified by reverse flash chromatography ((Column: Xbridge Phenyl OBD Column, 19*150 mm, 5 μm; Mobile Phase A: water (10 mmol/L NH₄HCO₃+0.05% NH₃H₂0, Mobile Phase B: MeCN; Flow rate: 60 mL/min; Gradient: 30% B to 40% B in 10 min; Wavelength: 254 nm/220 nm; RT1 (min) 10.788) to afford [(14R*,17S)-12,19-difluoro-14-{5-[2-(3-fluoroazetidin-1-yl)ethyl]-2-oxo-4-(trifluoromethyl)pyridin-1-yl}-20-methyl-15-oxo-3-(trifluoromethyl)-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid (31.9 mg, 25.4%) as a white solid. LC-MS: (ES+H, m/z) [M+H]⁺=754.20. ¹H NMR (300 MHz, CD₃OD) δ 7.97 (dd, J=6.1, 3.4 Hz, 1H), 7.70 (d, J=3.4 Hz, 2H), 7.61 (dt, J=9.1, 3.6 Hz, 1H), 7.35 (s, 1H), 7.21 (t, J=9.1 Hz, 1H), 7.07 (d, J=6.6 Hz, 1H), 6.88 (d, J=5.0 Hz, 2H), 6.60 (d, J=5.5 Hz, 1H), 6.01 (dd, J=6.1, 3.0 Hz, 1H), 5.73 (t, J=7.6 Hz, 1H), 5.10-4.85 (m, 1H), 3.54 (m, 2H), 3.28-3.12 (m, 2H), 2.86-2.54 (m, 4H), 2.49 (d, J=6.5 Hz, 2H), 2.37 (d, J=1.9 Hz, 3H). ¹⁹F NMR (282 MHz, CD₃OD) δ −59.08, −65.02, −122.31, −125.23, −180.50.

### Example 29

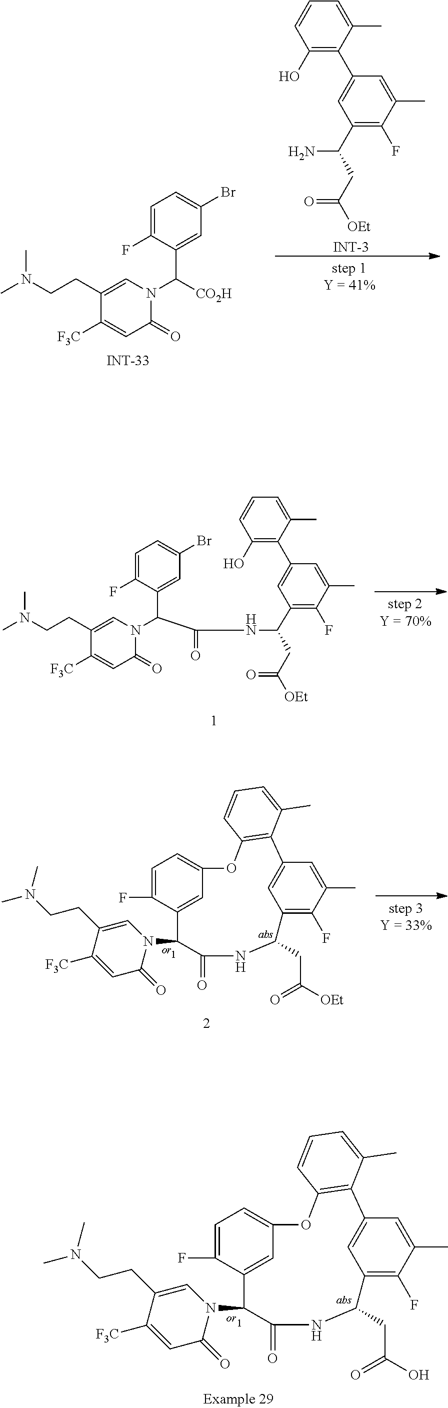

### Step 1: Preparation of ethyl (3S)-3-[2-(5-bromo-2-fluorophenyl)-2-{5-[2-(dimethylamino)ethyl]-2-oxo-4-(trifluoromethyl)pyridin-1-yl}acetamido]-3-{4-fluoro-2′-hydroxy-5,6′-dimethyl-[1,1′-biphenyl]-3-yl}propanoate

A solution of ethyl (3S)-3-amino-3-{4-fluoro-2′-hydroxy-5,6′-dimethyl-[1,1′-biphenyl]-3-yl}propanoate (299 mg, 0.90 mmol, 1.00 equiv.) and Et₃N (365 mg, 3.61 mmol, 4.00 equiv.) in CH₃CN (9 mL) was stirred for 1 h at 40° C. under nitrogen atmosphere, concentrated, and redissolved with CH₃CN (1 mL). In a separate vial, a solution of (5-bromo-2-fluorophenyl)({5-[2-(dimethylamino)ethyl]-2-oxo-4-(trifluoromethyl)pyridin-1-yl})acetic acid (420 mg, 0.90 mmol, 1.00 equiv.) and HOBT (182 mg, 1.35 mmol, 1.50 equiv.) in CH₃CN (9 mL) was treated with EDCI (346 mg, 1.80 mmol, 2.00 equiv.) in portions at room temperature under nitrogen atmosphere. The resulting mixture was stirred overnight at 40° C. under nitrogen atmosphere and then treated with the solution of (5-bromo-2-fluorophenyl)({5-[2-(dimethylamino)ethyl]-2-oxo-4-(trifluoromethyl)pyridin-1-yl})acetic acid in CH₃CN dropwise at 0° C. The resulting mixture was stirred overnight at 40° C., and then cooled to room temperature and concentrated. The residue was purified by reversed-phase flash chromatography (C18; mobile phase, MeCN in water (0.1% NH₃·H₂O), 50% to 75% gradient in 20 min; detector, UV 220 nm) to afford ethyl (3S)-3-[2-(5-bromo-2-fluorophenyl)-2-{5-[2-(dimethylamino)ethyl]-2-oxo-4-(trifluoromethyl)pyridin-1-yl}acetamido]-3-{4-fluoro-2′-hydroxy-5,6′-dimethyl-[1,1′-biphenyl]-3-yl}propanoate (248 mg, 41.4%) as a colorless oil. LC-MS: (ES+H, m/z) [M−H]⁻=778.10.

### Step 2: Preparation of ethyl 2-[(14R*,17S)-14-{5-[2-(dimethylamino)ethyl]-2-oxo-4-(trifluoromethyl)pyridin-1-yl}-12,19-difluoro-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate

To a stirred solution of ethyl (3S)-3-[2-(5-bromo-2-fluorophenyl)-2-{5-[2-(dimethylamino)ethyl]-2-oxo-4-(trifluoromethyl)pyridin-1-yl}acetamido]-3-{4-fluoro-2′-hydroxy-5,6′-dimethyl-[1,1′-biphenyl]-3-yl}propanoate (220 mg, 0.28 mmol, 1.00 equiv.) and (1R,2R)—N1,N2-dimethylcyclohexane-1,2-diamine (40 mg, 0.28 mmol, 1.00 equiv.) in 1,4-dioxane (5 mL) were added CuI (26 mg, 0.14 mmol, 0.50 equiv.) and K₂CO₃ (78 mg, 0.56 mmol, 2.00 equiv.) in portions at room temperature under nitrogen atmosphere. The resulting mixture was stirred overnight at 100° C. and then cooled to room temperature and concentrated. The residue was purified by reversed-phase flash chromatography (C18; mobile phase, MeCN in water (10 mM NH₄HCO₃), 50% to 75% gradient in 20 min; detector, UV 220 nm) to afford ethyl 2-[(14R*,17S)-14-{5-[2-(dimethylamino)ethyl]-2-oxo-4-(trifluoromethyl)pyridin-1-yl}-12,19-difluoro-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (138 mg, 70.0%) as a light yellow oil. LC-MS: (ES+H, m/z) [M+H]⁺=698.25.

### Step 3: Preparation of [(14R*,17S)-14-{5-[2-(dimethylamino)ethyl]-2-oxo-4-(trifluoromethyl)pyridin-1-yl}-12,19-difluoro-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid

To a stirred solution of ethyl 2-[(14R*,17S)-14-{5-[2-(dimethylamino)ethyl]-2-oxo-4-(trifluoromethyl)pyridin-1-yl}-12,19-difluoro-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (120 mg, 0.17 mmol, 1.00 equiv.) in THF (4 mL) was added LiOH·H₂O (8 mg, 0.34 mmol, 2.00 equiv.) in H₂O (1 mL) dropwise at 0° C. under nitrogen atmosphere. The resulting mixture was stirred overnight at 30° C. and then diluted with THF (10 mL), acidified to pH 5˜6 with 1 N HCl (aq.) and concentrated. The crude product (135 mg) was purified by Prep-HPLC (Column: YMC-Actus Triart C18 ExRS Column, 19*150 mm, 5 μm; Mobile Phase A: water (10 mmol/L NH₄HCO₃), Mobile Phase B: CH₃CN; Flow rate: 60 mL/min; Gradient: 22% B to 42% B in 10 min; Wavelength: 254 nm/220 nm; RT1 (min) 10.1), to afford [(14R*,17S)-14-{5-[2-(dimethylamino)ethyl]-2-oxo-4-(trifluoromethyl)pyridin-1-yl}-12,19-difluoro-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid (37.7 mg, 32.7%) as a white solid. LC-MS: (ES+H, m/z) [M+H]⁺=670.25. ¹H NMR (400 MHz, DMSO-d₆) δ 9.19 (d, J=7.0 Hz, 1H), 7.64-7.56 (m, 2H), 7.41-7.30 (m, 2H), 7.28-7.16 (m, 2H), 7.02 (dd, J=7.0, 2.1 Hz, 1H), 6.80 (s, 1H), 6.68 (s, 1H), 6.44 (dd, J=6.4, 2.2 Hz, 1H), 5.96 (dd, J=6.2, 2.9 Hz, 1H), 5.53-5.43 (m, 1H), 2.76-2.55 (m, 2H), 2.47-2.38 (m, 2H), 2.29 (s, 3H), 2.27-2.19 (m, 1H), 2.15-2.06 (m, 1H), 1.98-1.87 (m, 9H). ¹⁹F NMR (400 MHz, DMSO-d₆) −62.716, −121.833, −125.837.

### Example 30

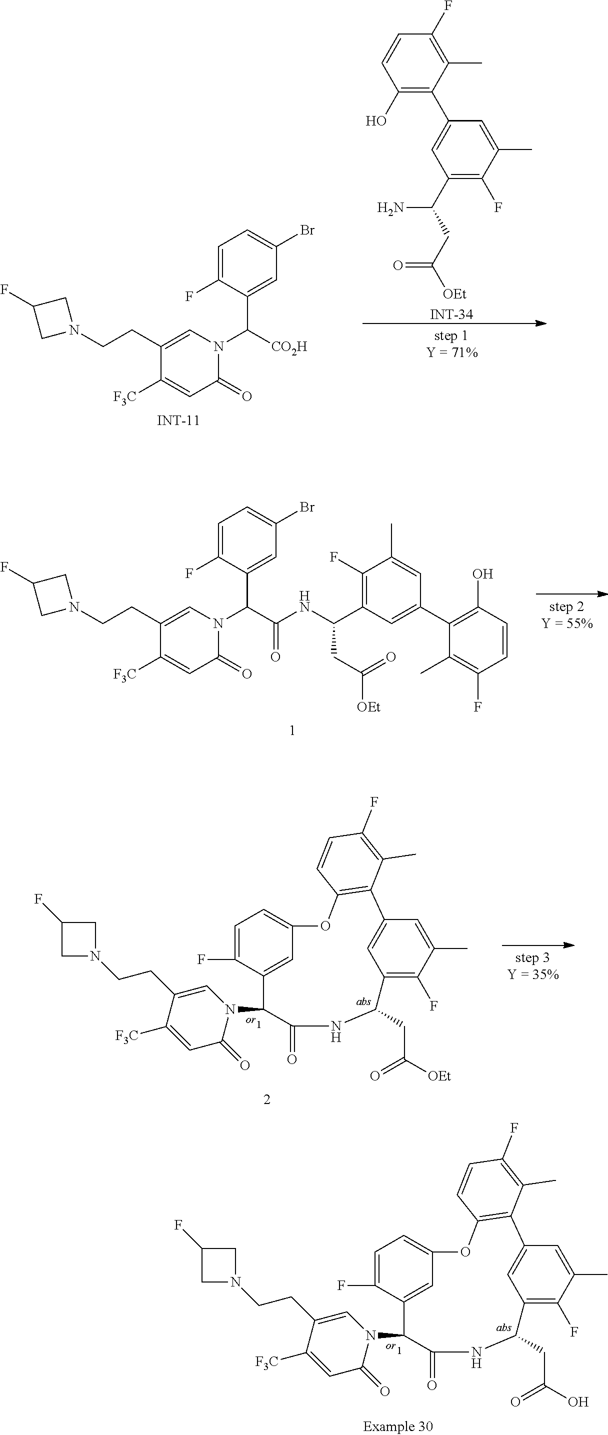

### Step 1: Preparation of ethyl (3S)-3-[2-(5-bromo-2-fluorophenyl)-2-{5-[2-(3-fluoroazetidin-1-yl)ethyl]-2-oxo-4-(trifluoromethyl)pyridin-1-yl}acetamido]-3-{3′,4-difluoro-6′-hydroxy-2′,5-dimethyl-[1,1′-biphenyl]-3-yl}propanoate

A solution of ethyl (3S)-3-amino-3-{3′,4-difluoro-6′-hydroxy-2′,5-dimethyl-[1,1′-biphenyl]-3-yl}propanoate (167 mg, 0.47 mmol, 1.00 equiv.) and triethylamine (483 mg, 4.78 mmol, 10.00 equiv.) in MeCN (4 mL) was stirred for 1 h at 40° C. under nitrogen atmosphere and then concentrated and then redissolved in MeCN (2 mL). In a separate vial, a stirred solution of (5-bromo-2-fluorophenyl)({5-[2-(3-fluoroazetidin-1-yl)ethyl]-2-oxo-4-(trifluoromethyl)pyridin-1-yl})acetic acid; trifluoroacetaldehyde (283 mg, 0.47 mmol, 1.00 equiv.) in MeCN (4 mL) was treated with HOBT (84 mg, 0.62 mmol, 1.30 equiv.) and EDCI (137 mg, 0.71 mmol, 1.50 equiv.) at room temperature under nitrogen atmosphere. The resulting mixture was stirred for an additional 1 h at 40° C. To the above mixture was added ethyl (3S)-3-amino-3-{3′,4-difluoro-6′-hydroxy-2′,5-dimethyl-[1,1′-biphenyl]-3-yl}propanoate in MeCN dropwise at 0° C. The resulting mixture was stirred overnight at 40° C., and then cooled to room temperature and concentrated. The residue was purified by reversed-phase flash chromatography (C18 gel; mobile phase, MeCN in water (0.1% NH₃·H₂O), 30% oto 70% gradient in 10 min; detector, UV 220 nm) to afford ethyl (3S)-3-[2-(5-bromo-2-fluorophenyl)-2-{5-[2-(3-fluoroazetidin-1-yl)ethyl]-2-oxo-4-(trifluoromethyl)pyridin-1-yl}acetamido]-3-{3′,4-difluoro-6′-hydroxy-2′,5-dimethyl-[1,1′-biphenyl]-3-yl}propanoate (281 mg, 71.1%) as a white solid. LC-MS: (ES+H, m/z) [M+H]⁺=826.1.

### Step 2: Preparation of ethyl 2-[(14R*,17S)-4,12,19-trifluoro-14-{5-[2-(3-fluoroazetidin-1-yl)ethyl]-2-oxo-4-(trifluoromethyl)pyridin-1-yl}-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate

To a stirred mixture of ethyl (3S)-3-[2-(5-bromo-2-fluorophenyl)-2-{5-[2-(3-fluoroazetidin-1-yl)ethyl]-2-oxo-4-(trifluoromethyl)pyridin-1-yl}acetamido]-3-{3′,4-difluoro-6′-hydroxy-2′,5-dimethyl-[1,1′-biphenyl]-3-yl}propanoate (281 mg, 0.34 mmol, 1.00 equiv.) and (1R,2R)—N1,N2-dimethylcyclohexane-1,2-diamine (58 mg, 0.40 mmol, 1.20 equiv.) in dioxane (10 mL) were added CuI (19 mg, 0.10 mmol, 0.30 equiv.) and K₂CO₃ (93 mg, 0.68 mmol, 2.00 equiv.) at room temperature under nitrogen atmosphere. The resulting mixture was stirred overnight at 100° C., and then cooled to room temperature and filtered. The filter cake was washed with EtOAc (3×100 mL) and the filtrate was concentrated under reduced pressure. The residue was purified by reversed-phase flash chromatography (C18 gel; mobile phase, MeCN in water (0.11% NH₃·H₂O), 30% to 60% gradient in 30 min; detector, UV 220 nm) to afford ethyl 2-[(14R*,17S)-4,12,19-trifluoro-14-{5-[2-(3-fluoroazetidin-1-yl)ethyl]-2-oxo-4-(trifluoromethyl)pyridin-1-yl}-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (140 mg, 55.2%) as a red solid. LC-MS: (ES+H, m/z) [M+H]⁺=746.3.

### Step 3: Preparation of [(14R*,17S)-4,12,19-trifluoro-14-{5-[2-(3-fluoroazetidin-1-yl)ethyl]-2-oxo-4-(trifluoromethyl)pyridin-1-yl}-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid

To a stirred mixture of ethyl 2-[(14R*,17S)-4,12,19-trifluoro-14-{5-[2-(3-fluoroazetidin-1-yl)ethyl]-2-oxo-4-(trifluoromethyl)pyridin-1-yl}-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (120 mg, 0.16 mmol, 1.00 equiv.) and H₂O (1 mL) in THF (3 mL) was added LiOH·H₂O (13 mg, 0.32 mmol, 2.00 equiv.) at room temperature under nitrogen atmosphere. The resulting mixture was stirred for 1 h at room temperature, and then acidified to pH 5 with 1 M HCl (aq.), and concentrated. The crude product was purified by Prep-HPLC (Column: XSelect CSH Prep C18 OBD Column, 30*150 mm, 5 μm; Mobile Phase A: water (0.1% HCOOH), Mobile Phase B: MeCN; Flow rate: 60 mL/min; Gradient: 25% B to 38% B in 10 min; Wavelength: 254 nm/220 nm; RT1 (min) 7.17) to afford [(14R*,17S)-4,12,19-trifluoro-14-{5-[2-(3-fluoroazetidin-1-yl)ethyl]-2-oxo-4-(trifluoromethyl)pyridin-1-yl}-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid (42.1 mg, 34.6%) as a white solid. LC-MS: (ES+H, m/z) [M+H]⁺=718.20. ¹H NMR (300 MHz, DMSO-d₆) δ 12.98-11.98 (m, 1H), 9.18 (d, J=7.3 Hz, 1H), 7.71 (dd, J=8.9, 4.8 Hz, 1H), 7.63 (dt, J=9.0, 3.6 Hz, 1H), 7.30 (td, J=9.0, 3.3 Hz, 2H), 7.23 (s, 1H), 7.07 (dd, J=6.9, 2.2 Hz, 1H), 6.81 (s, 1H), 6.70 (s, 1H), 6.46 (dd, J=6.4, 2.2 Hz, 1H), 5.96 (dd, J=6.2, 2.9 Hz, 1H), 5.49 (q, J=7.5 Hz, 1H), 5.00-4.70 (m, 1H), 3.30-3.19 (m, 2H), 2.95-2.78 (m, 2H), 2.74-2.61 (m, 2H), 2.42-2.26 (m, 7H), 1.86 (d, J=2.3 Hz, 3H). ¹⁹F NMR (282 MHz, DMSO-d₆) δ −62.74, −118.82, −121.49, −125.10, −177.02.

### Example 31

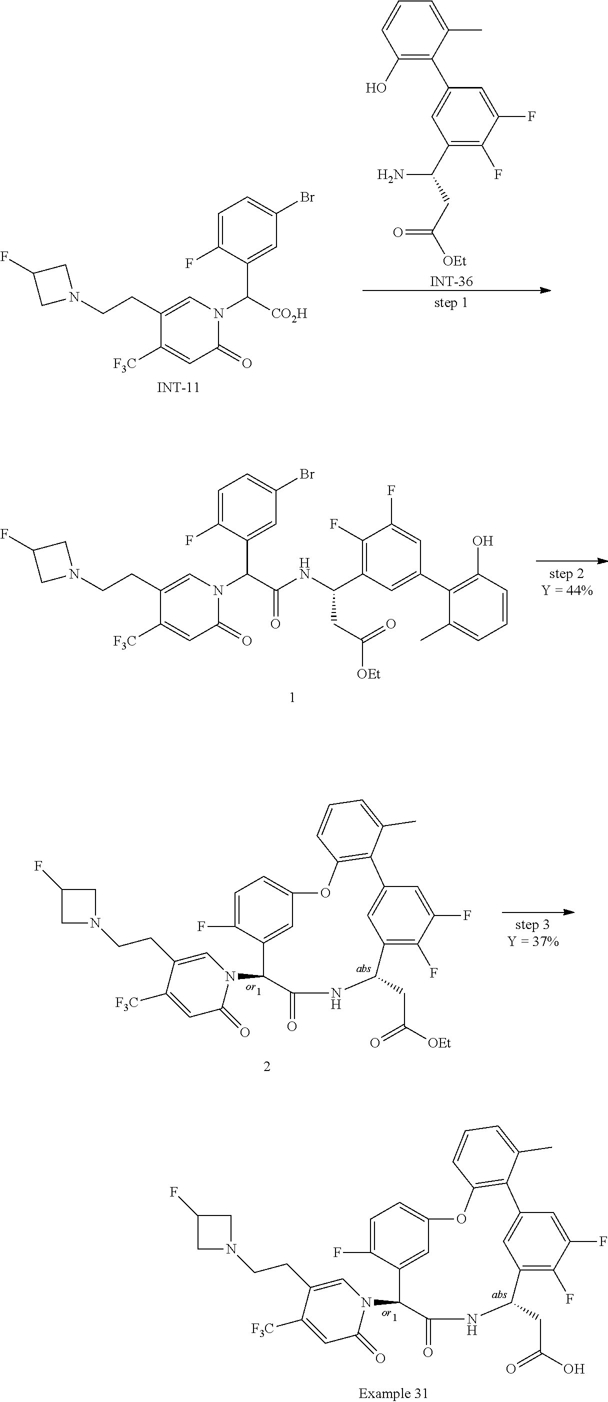

### Step 1: Preparation of ethyl (3S)-3-[2-(5-bromo-2-fluorophenyl)-2-{5-[2-(3-fluoroazetidin-1-yl)ethyl]-2-oxo-4-(trifluoromethyl)pyridin-1-yl}acetamido]-3-{4,5-difluoro-2′-hydroxy-6′-methyl-[1,1′-biphenyl]-3-yl}propanoate

A mixture of (5-bromo-2-fluorophenyl)({5-[2-(3-fluoroazetidin-1-yl)ethyl]-2-oxo-4-(trifluoromethyl)pyridin-1-yl})acetic acid (300 mg, 0.60 mmol, 1.00 equiv.), EDCI (185 mg, 0.97 mmol, 1.60 equiv.) and HOBT (114 mg, 0.84 mmol, 1.40 equiv.) in MeCN (6 mL) was stirred for 2 h at 30° C. under nitrogen atmosphere. To the stirred mixture was added a solution of ethyl (3S)-3-amino-3-{4,5-difluoro-2′-hydroxy-6′-methyl-[1,1′-biphenyl]-3-yl}propanoate hydrochloride (270 mg, 0.72 mmol, 1.20 equiv.) and NEt₃ (367 mg, 3.63 mmol, 6.00 equiv.) in MeCN (6 mL) dropwise at 30° C. The resulting mixture was stirred overnight at 40° C. and then cooled to room temperature, diluted with water (50 mL) and extracted with EtOAc (3×50 mL). The combined organic layers were washed with brine, dried over anhydrous Na₂SO₄, filtered, and concentrated. The residue was purified by reversed-phase flash chromatography (C18 gel; mobile phase, MeCN in water (0.1% NH₃·H₂O), 30% to 50% gradient in 30 min; detector, UV 254 nm) to provide ethyl (3S)-3-[2-(5-bromo-2-fluorophenyl)-2-{5-[2-(3-fluoroazetidin-1-yl)ethyl]-2-oxo-4-(trifluoromethyl)pyridin-1-yl}acetamido]-3-{4,5-difluoro-2′-hydroxy-6′-methyl-[1,1′-biphenyl]-3-yl}propanoate (250 mg, 32.6% two steps, mixture) as a white solid. LC-MS: (ES+H, m/z) [M+H]⁺=812.2.

### Step 2: Preparation of ethyl 2-[(14R*,17S)-12,19,20-trifluoro-14-{5-[2-(3-fluoroazetidin-1-yl)ethyl]-2-oxo-4-(trifluoromethyl)pyridin-1-yl}-3-methyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate

A mixture of ethyl (3S)-3-[2-(5-bromo-2-fluorophenyl)-2-{5-[2-(3-fluoroazetidin-1-yl)ethyl]-2-oxo-4-(trifluoromethyl)pyridin-1-yl}acetamido]-3-{4,5-difluoro-2′-hydroxy-6′-methyl-[1,1′-biphenyl]-3-yl}propanoate (250 mg, 0.31 mmol, 1.00 equiv., mixture), N₁,N₂-dimethylcyclohexane-1,2-diamine (43 mg, 0.31 mmol, 1.00 equiv.), CuI (29 mg, 0.15 mmol, 0.50 equiv.) and K₂CO₃ (85 mg, 0.62 mmol, 2.00 equiv.) in 1,4-dioxane (6 mL) was stirred overnight at 100° C. under nitrogen atmosphere and then cooled to room temperature and concentrated. The resulting mixture was diluted with water (10 mL) and extracted with EtOAc (3×10 mL). The combined organic layers were washed with brine, dried over anhydrous Na₂SO₄, filtered, and concentrated. The residue was purified by Prep-TLC (CH₂Cl₂/MeOH (0.11% NH₃·H₂O) 20:1) to afford ethyl 2-[(14R*,17S)-12,19,20-trifluoro-14-{5-[2-(3-fluoroazetidin-1-yl)ethyl]-2-oxo-4-(trifluoromethyl)pyridin-1-yl}-3-methyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (100 mg, 44.4%) as a white solid. LC-MS: (ES+H, m/z) [M+H]⁺=732.2.

### Step 3: Preparation of [(14R*,17S)-12,19,20-trifluoro-14-{5-[2-(3-fluoroazetidin-1-yl)ethyl]-2-oxo-4-(trifluoromethyl)pyridin-1-yl}-3-methyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid

To a stirred solution of ethyl 2-[(14R*,17S)-12,19,20-trifluoro-14-{5-[2-(3-fluoroazetidin-1-yl)ethyl]-2-oxo-4-(trifluoromethyl)pyridin-1-yl}-3-methyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (100 mg, 0.13 mmol, 1.00 equiv.) in THF (2 mL) was added LiOH·H₂O (29 mg, 0.68 mmol, 5.00 equiv.) in H₂O (0.5 mL) dropwise at room temperature under nitrogen atmosphere. After 3 h the reaction mixture was neutralized to pH 7 with HCl (2 M, aq.) and then concentrated. The residue was purified by reverse flash chromatography ((Column: Xselect CSH C18 OBD Column 30*150 mm, 5 μm; Mobile Phase A: water (0.1% HCOOH), Mobile Phase B: MeCN; Flow rate: 60 mL/min; Gradient: 20% B to 37% B in 8 min; Wavelength: 254 nm/220 nm; RT1 (min) 9.58) to afford [(14R*,17S)-12,19,20-trifluoro-14-{5-[2-(3-fluoroazetidin-1-yl)ethyl]-2-oxo-4-(trifluoromethyl)pyridin-1-yl}-3-methyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid (35.7 mg, 37.1%) as a white solid. LC-MS: (ES+H, m/z) [M+H]⁺=704.10. ¹H NMR (400 MHz, CD₄OD) δ 7.57 (dd, J=8.1, 4.1 Hz, 1H), 7.47 (d, J=8.1 Hz, 1H), 7.43-7.32 (m, 2H), 7.17 (ddd, J=8.9, 6.9, 2.5 Hz, 2H), 7.09-7.00 (m, 1H), 6.87 (d, J=16.0 Hz, 2H), 6.46 (d, J=5.2 Hz, 1H), 6.05 (dd, J=6.1, 3.0 Hz, 1H), 5.65 (td, J=7.6, 3.0 Hz, 1H), 5.20-4.90 (m, 1H), 3.74 (m, 2H), 3.42 (m, 2H), 2.88-2.63 (m, 4H), 2.53 (m, J=6.9 Hz, 2H), 2.02 (s, 3H).

### Example 32

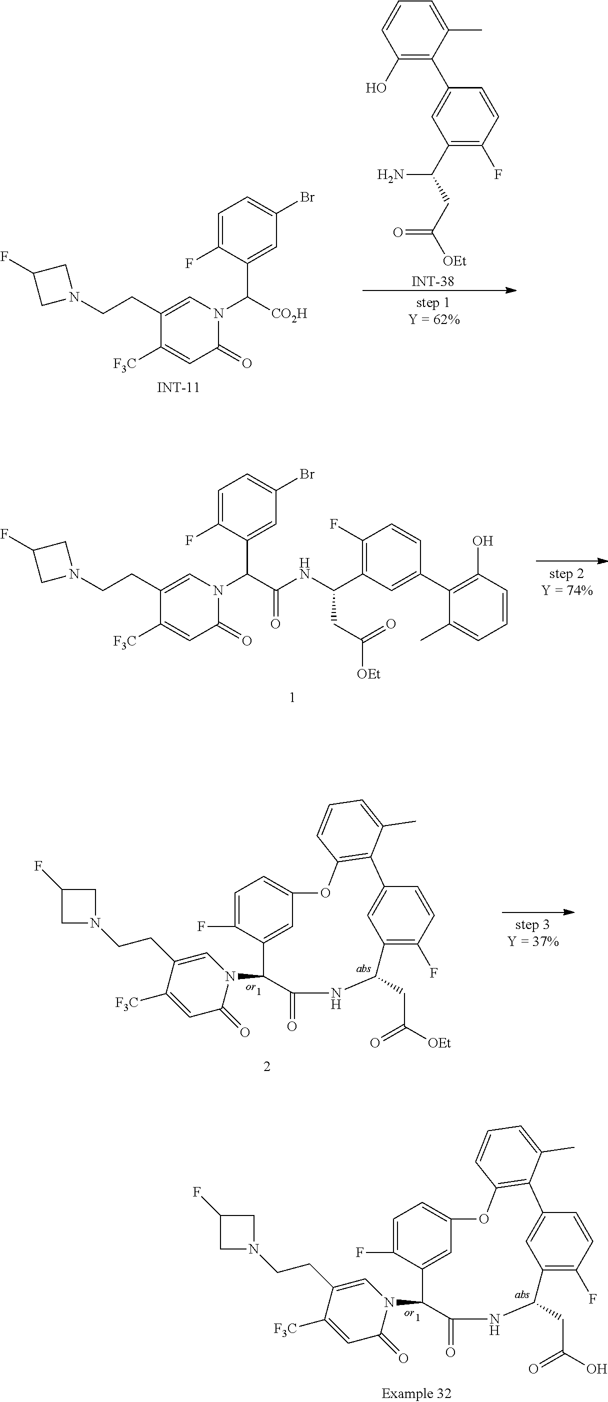

### Step 1: Preparation of ethyl (3S)-3-[2-(5-bromo-2-fluorophenyl)-2-{5-[2-(3-fluoroazetidin-1-yl)ethyl]-2-oxo-4-(trifluoromethyl)pyridin-1-yl}acetamido]-3-{4-fluoro-2′-hydroxy-6′-methyl-[1,1′-biphenyl]-3-yl}propanoate

A mixture of (5-bromo-2-fluorophenyl)({5-[2-(3-fluoroazetidin-1-yl)ethyl]-2-oxo-4-(trifluoromethyl)pyridin-1-yl})acetic acid (200 mg, 0.40 mmol, 1.00 equiv.), EDCI (155 mg, 0.81 mmol, 2.00 equiv.) and HOBT (109 mg, 0.81 mmol, 2.00 equiv.) in MeCN (10 mL) was stirred for 1 h at 40° C. under nitrogen atmosphere. To the above mixture was added ethyl (3S)-3-amino-3-{4-fluoro-2′-hydroxy-6′-methyl-[1,1′-biphenyl]-3-yl}propanoate (141 mg, 0.44 mmol, 1.10 equiv.) in CH₃CN (5 mL) dropwise at 0° C. The resulting mixture was stirred for an overnight at 40° C. and then concentrated under reduced pressure. The residue was purified by reversed-phase flash chromatography (C18 gel; mobile phase, MeCN in water (0.1% NH₃·H₂O), 40% to 70% gradient in 15 min; detector, UV 220 nm) to provide ethyl (3S)-3-[2-(5-bromo-2-fluorophenyl)-2-{5-[2-(3-fluoroazetidin-1-yl)ethyl]-2-oxo-4-(trifluoromethyl)pyridin-1-yl}acetamido]-3-{4-fluoro-2′-hydroxy-6′-methyl-[1,1′-biphenyl]-3-yl}propanoate (200 mg, 62.3%, mixture) as a colorless oil. LC-MS: (ES+H, m/z) [M+H]⁺=794.05.

### Step 2: Preparation of ethyl 2-((5S,8S)-1⁴,4⁴-difluoro-5-(5-(2-(3-fluoroazetidin-1-yl) ethyl)-2-oxo-4-(trifluoromethyl) pyridin-1(2H)-yl)-2⁶-methyl-6-oxo-3-oxa-7-aza-1,4(1,3),2(1,2)-tribenzenacyclooctaphane-8-yl) acetate

To a stirred solution of ethyl (3S)-3-[2-(5-bromo-2-fluorophenyl)-2-{5-[2-(3-fluoroazetidin-1-yl) ethyl]-2-oxo-4-(trifluoromethyl) pyridin-1-yl} acetamido]-3-{4-fluoro-2′-hydroxy-6′-methyl-[1,1′-biphenyl]-3-yl} propanoate (210 mg, 0.26 mmol, 1.00 equiv., mixture) in 1,4-dioxane (5 mL) was added CuI (25 mg, 0.13 mmol, 0.50 equiv.), K₂CO₃ (73 mg, 0.52 mmol, 2.00 equiv.) and (1R,2R)—N1,N2-dimethylcyclohexane-1,2-diamine (37 mg, 0.26 mmol, 1.00 equiv.) at room temperature under air atmosphere. The resulting mixture was stirred overnight at 100° C. under nitrogen atmosphere, and then cooled to room temperature and concentrated. The residue was purified by Prep-TLC (CH₂Cl₂/MeOH 20:1, Rf=0.3) to afford ethyl 2-((5S,8S)-1⁴,4⁴-difluoro-5-(5-(2-(3-fluoroazetidin-1-yl)ethyl)-2-oxo-4-(trifluoromethyl)pyridin-1(2H)-yl)-2⁶-methyl-6-oxo-3-oxa-7-aza-1,4(1,3),2(1,2)-tribenzenacyclooctaphane-8-yl)acetate (140 mg, 74.2%) as a white solid. LC-MS: (ES+H, m/z) [M+H]⁺=714.25. ¹H NMR (400 MHz, CDCl₃) δ 7.58 (d, J=7.8 Hz, 1H), 7.43-7.31 (m, 3H), 7.22-7.04 (m, 5H), 6.86 (d, J=9.4 Hz, 2H), 6.65-6.56 (m, 1H), 6.38-6.28 (m, 1H), 5.76 (q, J=6.0 Hz, 1H), 4.12-3.92 (m, 2H), 3.44 (s, 2H), 3.20-2.77 (m, 5H), 1.97 (s, 2H), 1.31-1.23 (m, 3H), 1.15 (d, J=7.2 Hz, 2H), 0.95-0.79 (m, 3H). ¹⁹F NMR (377 MHz, CDCl₃) δ −63.81, −120.28, −120.86, −178.20.

### Step 3: Preparation of 2-((5S,8S)-1⁴,4⁴-difluoro-5-(5-(2-(3-fluoroazetidin-1-yl)ethyl)-2-oxo-4-(trifluoromethyl)pyridin-1(2H)-yl)-2⁶-methyl-6-oxo-3-oxa-7-aza-1,4(1,3),2(1,2)-tribenzenacyclooctaphane-8-yl)acetic acid

To a stirred solution of ethyl 2-[(14S,17S)-12,19-difluoro-14-{5-[2-(3-fluoroazetidin-1-yl) ethyl]-2-oxo-4-(trifluoromethyl) pyridin-1-yl}-3-methyl-15-oxo-8-oxa-16-azatetracyclo [16.3.1.1\hat{ }{9,13}.0\hat{ }{2,7}] tricosa-1(22),2(7),3,5,9,11,13(23),18,20-nonaen-17-yl] acetate (120 mg, 0.16 mmol, 1.00 equiv.) in THF:H₂O=5:1 (6 mL) was added LiOH·H₂O (28 mg, 0.67 mmol, 4.00 equiv.) at room temperature under nitrogen atmosphere. The resulting mixture was stirred for 2 h at 30° C. and then acidified to pH 5 with 1 M. HCl (aq.) and concentrated. The residue was purified by reversed-phase flash chromatography (Column: XBridge Prep OBD C18 Column, 30*150 mm, 10 μm; Mobile Phase A: water (10 mmol/L NH₄HCO₃+0.05% NH₃H₂O), Mobile Phase B: MeCN; Flow rate: 60 mL/min; Gradient: 29% B to 40% B in 8 min; Wavelength: 254 nm/220 nm; RT1 (min) 9.18) to afford 2-((5S,8S)-14,44-difluoro-5-(5-(2-(3-fluoroazetidin-1-yl)ethyl)-2-oxo-4-(trifluoromethyl)pyridin-1(2H)-yl)-26-methyl-6-oxo-3-oxa-7-aza-1,4(1,3),2(1,2)-tribenzenacyclooctaphane-8-yl)acetic acid (43.8 mg, 37.0%) as a white solid. LC-MS: (ES+H, m/z) [M+H]⁺=686.05. ¹H NMR (400 MHz, MeOD) δ 7.59-7.53 (m, 1H), 7.48-7.42 (m, 1H), 7.38-7.30 (m, 2H), 7.26-7.03 (m, 4H), 6.86 (d, J=3.6 Hz, 2H), 6.70-6.65 (m, 1H), 6.09 (q, J=2.4 Hz, 1H), 5.68 (t, J=7.5 Hz, 1H), 5.11-4.88 (m, 1H), 3.59 (s, 2H), 3.28-3.11 (m, 2H), 2.83-2.57 (m, 4H), 2.48 (d, J=6.3 Hz, 2H), 1.99 (s, 3H). ¹⁹F NMR (377 MHz, MeOD) δ −64.98, −122.16, −123.12, −180.40.

### Example 33

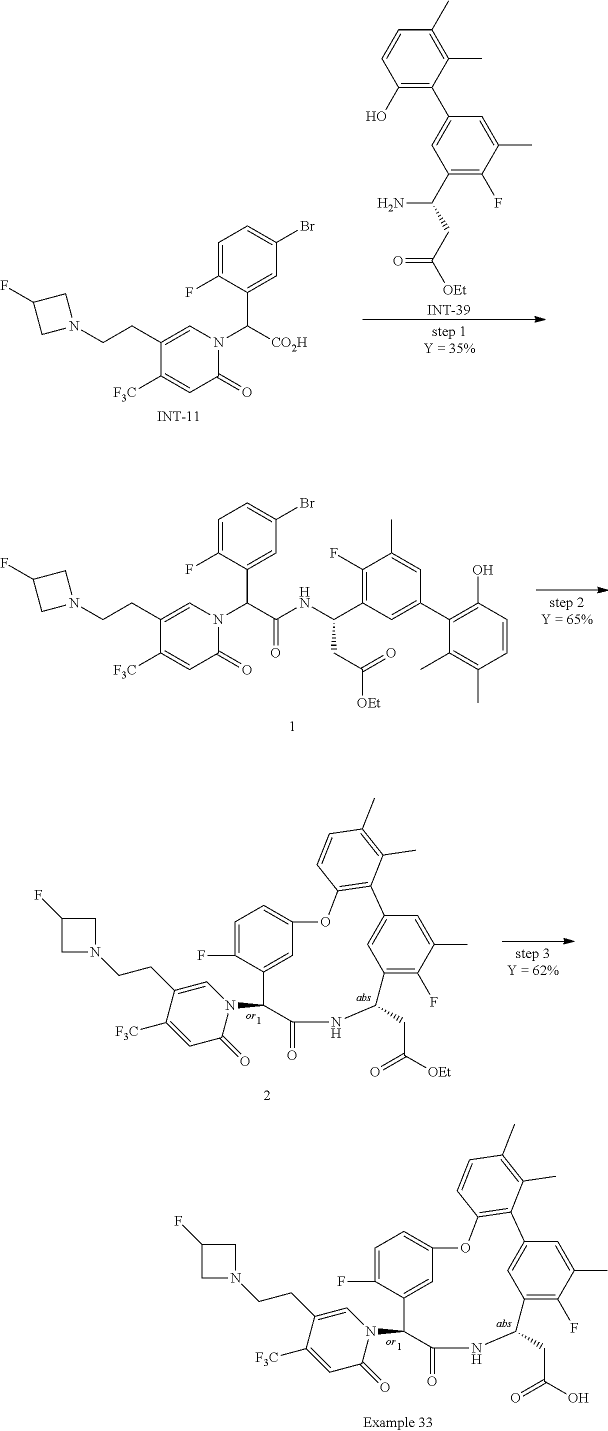

### Step 1: Preparation of ethyl (3S)-3-[2-(5-bromo-2-fluorophenyl)-2-{5-[2-(3-fluoroazetidin-1-yl)ethyl]-2-oxo-4-(trifluoromethyl)pyridin-1-yl}acetamido]-3-{4-fluoro-6′-hydroxy-2′,3′,5-trimethyl-[1,1′-biphenyl]-3-yl}propanoate

A solution of ethyl (3S)-3-amino-3-{4-fluoro-6′-hydroxy-2′,3′,5-trimethyl-[1,1′-biphenyl]-3-yl}propanoate hydrochloride (130 mg, 0.34 mmol, 1.00 equiv.) and NEt₃ (344 mg, 3.40 mmol, 10.00 equiv.) in CH₃CN (1 mL) was stirred for 30 min at 40° C. under nitrogen atmosphere and then concentrated under reduced pressure. In a separate flask, a stirred mixture of (5-bromo-2-fluorophenyl)({5-[2-(3-fluoroazetidin-1-yl)ethyl]-2-oxo-4-(trifluoromethyl)pyridin-1-yl})acetic acid; trifluoroacetaldehyde (202 mg, 0.34 mmol, 1.00 equiv.) and HOBT (60 mg, 0.44 mmol, 1.30 equiv.) in CH₃CN (1 mL) was treated with EDCI (98 mg, 0.51 mmol, 1.50 equiv.) at room temperature under nitrogen atmosphere. The resulting mixture was stirred for 1 h at 40° C. under nitrogen atmosphere and then ethyl (3S)-3-amino-3-{4-fluoro-6′-hydroxy-2′,3′,5-trimethyl-[1,1′-biphenyl]-3-yl}propanoate hydrochloride (130 mg, 0.34 mmol, 1.00 equiv.) in CH₃CN (1 mL) dropwise at 0° C. The resulting mixture was stirred overnight at 40° C., concentrated, and then purified by reversed-phase flash chromatography (C18 gel; mobile phase, CH₃CN in water (0.1% NH₃·H₂O), 20% to 100% gradient in 30 min; detector, UV 220 nm) to provide ethyl (3S)-3-[2-(5-bromo-2-fluorophenyl)-2-{5-[2-(3-fluoroazetidin-1-yl)ethyl]-2-oxo-4-(trifluoromethyl)pyridin-1-yl}acetamido]-3-{4-fluoro-6′-hydroxy-2′,3′,5-trimethyl-[1,1′-biphenyl]-3-yl}propanoate (100 mg, 35.7%) as a light yellow solid. LC-MS: (ES+H, m/z) [M+H]⁺=822.1. ¹H NMR (300 MHz, DMSO-d₆) δ 9.34 (s, 1H), 8.81 (s, 1H), 7.82-7.50 (m, 2H), 7.38-7.16 (m, 3H), 7.04-6.89 (m, 3H), 6.87-6.71 (m, 2H), 6.65 (d, J=7.9 Hz, 1H), 5.59 (d, J=31.1 Hz, 1H), 4.01 (q, 2H), 3.01-2.58 (m, 5H), 2.38 (d, J=13.0 Hz, 3H), 2.31-2.21 (m, 3H), 2.19-2.04 (m, 4H), 1.89-1.67 (m, 3H), 1.08 (dd, J=8.1, 6.1 Hz, 3H).

### Step 2: Preparation of ethyl 2-[(14R*,17S)-12,19-difluoro-14-{5-[2-(3-fluoroazetidin-1-yl)ethyl]-2-oxo-4-(trifluoromethyl)pyridin-1-yl}-3,4,20-trimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate

To a stirred solution of ethyl (3S)-3-[2-(5-bromo-2-fluorophenyl)-2-{5-[2-(3-fluoroazetidin-1-yl)ethyl]-2-oxo-4-(trifluoromethyl)pyridin-1-yl}acetamido]-3-{4-fluoro-6′-hydroxy-2′,3′,5-trimethyl-[1,1′-biphenyl]-3-yl}propanoate (120 mg, 0.15 mmol, 1.00 equiv., mixture) and (1R,2R)—N1,N2-dimethylcyclohexane-1,2-diamine (25 mg, 0.18 mmol, 1.20 equiv.) in dioxane (10 mL) were added CuI (8 mg, 0.04 mmol, 0.30 equiv.) and K₂CO₃ (40 mg, 0.29 mmol, 2.00 equiv.) at room temperature under nitrogen atmosphere. The resulting mixture was stirred overnight at 100° C., cooled to room temperature, and filtered. The filter cake was washed with EtOAc (3×10 mL) and the combined filtrate was concentrated under reduced pressure. The residue was purified by reversed-phase flash chromatography (C18 gel; mobile phase, CH₃CN in water (0.10% NH₃·H₂O), 50% to 80% gradient in 30 min; detector, UV 254 nm) to provide ethyl 2-[(14R*,17S)-12,19-difluoro-14-{5-[2-(3-fluoroazetidin-1-yl)ethyl]-2-oxo-4-(trifluoromethyl)pyridin-1-yl}-3,4,20-trimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (70 mg, 64.7%) as a light yellow solid. LC-MS: (ES+H, m/z) [M+H]⁺=742.3.

### Step 3: Preparation of [(14R*,17S)-12,19-difluoro-14-{5-[2-(3-fluoroazetidin-1-yl)ethyl]-2-oxo-4-(trifluoromethyl)pyridin-1-yl}-3,4,20-trimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid

To a stirred solution of ethyl 2-[(14R*,17S)-12,19-difluoro-14-{5-[2-(3-fluoroazetidin-1-yl)ethyl]-2-oxo-4-(trifluoromethyl)pyridin-1-yl}-3,4,20-trimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (70 mg, 0.09 mmol, 1.00 equiv.) in THF (2 mL) and H₂O (1 mL) were added LiOH·H₂O (0.1 mL, 2 M, 0.19 mmol, 2.00 equiv.) dropwise at room temperature. The resulting mixture was stirred for 1 h at room temperature, and then acidified to pH 3 with 2 M HCl (aq.), and concentrated. The crude product (120 mg) was purified by Prep-HPLC (Column: XSelect CSH Prep C18 OBD Column, 30*150 mm, 5 μm; Mobile Phase A: water (0.1% HCOOH), Mobile Phase B: CH₃CN; Flow rate: 60 mL/min; Gradient: 20% B to 40% B in 10 min; Wavelength: 254 nm/220 nm; RT1 (min) 9.7) to afford [(14R*,17S)-12,19-difluoro-14-{5-[2-(3-fluoroazetidin-1-yl)ethyl]-2-oxo-4-(trifluoromethyl)pyridin-1-yl}-3,4,20-trimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid (42.5 mg, 62.1%) as a white solid. LC-MS: (ES+H, m/z) [M+H]⁺=714.20. ¹H NMR (300 MHz, DMSO-d₆) δ 9.18 (d, J=7.3 Hz, 1H), 7.62-7.55 (m, 1H), 7.51 (d, J=8.2 Hz, 1H), 7.30-7.20 (m, 3H), 6.99 (dd, J=6.7, 2.2 Hz, 1H), 6.81 (s, 1H), 6.69 (s, 1H), 6.41 (dd, J=6.5, 2.2 Hz, 1H), 5.93 (dd, J=6.3, 2.9 Hz, 1H), 5.49 (q, 1H), 4.99-4.71 (m, 1H), 3.22 (dd, J=14.5, 6.9 Hz, 2H), 2.94-2.75 (m, 2H), 2.66 (dd, J=7.7, 4.2 Hz, 2H), 2.44-2.28 (m, 7H), 2.25 (s, 3H), 1.84 (s, 3H). ¹⁹F NMR (282 MHz, DMSO-d₆) δ −62.611, −121.916, −125.982, −177.029.

### Example 34

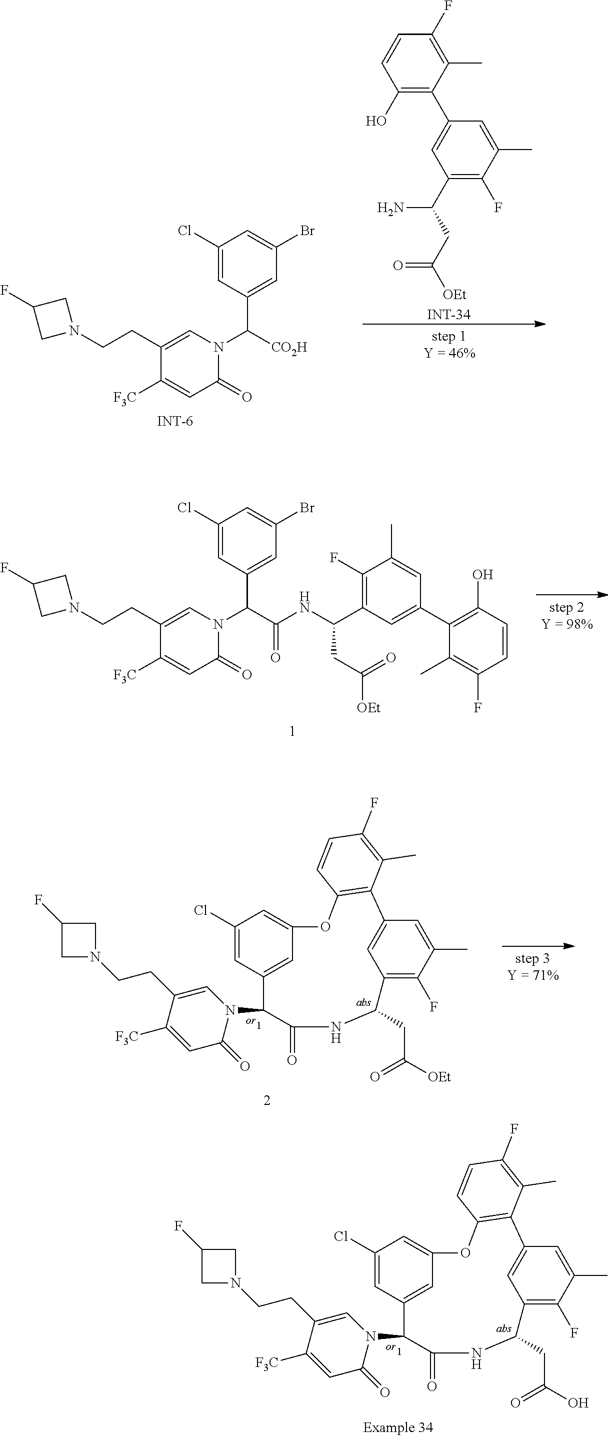

### Step 1: Preparation of ethyl (3S)-3-[2-(3-bromo-5-chlorophenyl)-2-{5-[2-(3-fluoroazetidin-1-yl)ethyl]-2-oxo-4-(trifluoromethyl)pyridin-1-yl}acetamido]-3-{3′,4-difluoro-6′-hydroxy-2′,5-dimethyl-[1,1′-biphenyl]-3-yl}propanoate

A solution of ethyl (S)-3-amino-3-(3′,4-difluoro-6′-hydroxy-2′,5-dimethyl-[1,1′-biphenyl]-3-yl)propanoate hydrochloride (100 mg, 0.25 mmol, 1.00 equiv.) and triethylamine (262 mg, 2.59 mmol, 10.00 equiv.) in MeCN (4 mL) was stirred for 1 h at 40° C. under nitrogen atmosphere. The resulting mixture was concentrated under reduced pressure and redissolved in MeCN (2 mL). In a separate vial, a solution of (3-bromo-5-chlorophenyl)({5-[2-(3-fluoroazetidin-1-yl)ethyl]-2-oxo-4-(trifluoromethyl)pyridin-1-yl})acetic acid (133 mg, 0.26 mmol, 1.00 equiv.) in MeCN (4 mL) was treated with HOBT (45 mg, 0.33 mmol, 1.30 equiv.) and EDCI (74 mg, 0.38 mmol, 1.50 equiv.) at room temperature under nitrogen atmosphere. The resulting mixture was stirred for an additional 1 h at 40° C. To the above mixture was added ethyl (3S)-3-amino-3-{3′,4-difluoro-6′-hydroxy-2′,5-dimethyl-[1,1′-biphenyl]-3-yl}propanoate in MeCN (2 mL) dropwise at 0° C. The resulting mixture was stirred overnight at 40° C., and then concentrated, and purified by reversed-phase flash chromatography (C18 gel; mobile phase, MeCN in water (0.1% NH₃·H₂O), 30% to 70% gradient in 10 min; detector, UV 220 nm) to afford ethyl (3S)-3-[2-(3-bromo-5-chlorophenyl)-2-{5-[2-(3-fluoroazetidin-1-yl)ethyl]-2-oxo-4-(trifluoromethyl)pyridin-1-yl}acetamido]-3-{3′,4-difluoro-6′-hydroxy-2′,5-dimethyl-[1,1′-biphenyl]-3-yl}propanoate (100 mg, 45.7%, mixture) as a white solid. LC-MS: (ES+H, m/z) [M+H]⁺=842.2.

### Step 2: Preparation of ethyl 2-[(14S,17S)-11-chloro-4,19-difluoro-14-{5-[2-(3-fluoroazetidin-1-yl)ethyl]-2-oxo-4-(trifluoromethyl)pyridin-1-yl}-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate

To a stirred mixture of ethyl (3S)-3-[2-(3-bromo-5-chlorophenyl)-2-{5-[2-(3-fluoroazetidin-1-yl)ethyl]-2-oxo-4-(trifluoromethyl)pyridin-1-yl}acetamido]-3-{3′,4-difluoro-6′-hydroxy-2′,5-dimethyl-[1,1′-biphenyl]-3-yl}propanoate (190 mg, 0.22 mmol, 1.00 equiv., mixture) and CuI (6 mg, 0.03 mmol, 0.30 equiv.) in dioxane (10 mL) were added (1R,2R)—N1,N2-dimethylcyclohexane-1,2-diamine (18 mg, 0.12 mmol, 1.20 equiv.) and K₂CO₃ (29 mg, 0.21 mmol, 2.00 equiv.) at room temperature under nitrogen atmosphere. The resulting mixture was stirred overnight at 100° C., and then cooled to room temperature and filtered. The filter cake was washed with EtOAc (3×100 mL) and the combined filtrate was concentrated under reduced pressure. The residue was purified by reversed-phase flash chromatography (C18 gel; mobile phase, MeCN in water (0.1% NH₃·H₂O), 30% to 60% gradient in 30 min; detector, UV 220 nm) to afford ethyl 2-[(14S,17S)-11-chloro-4,19-difluoro-14-{5-[2-(3-fluoroazetidin-1-yl)ethyl]-2-oxo-4-(trifluoromethyl)pyridin-1-yl}-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (80 mg, 98.3%) as a white solid. LC-MS: (ES+H, m/z) [M+H]⁺=762.3.

### Step 3: Preparation of [(14R*,17S)-11-chloro-4,19-difluoro-14-{5-[2-(3-fluoroazetidin-1-yl)ethyl]-2-oxo-4-(trifluoromethyl)pyridin-1-yl}-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid

To a stirred mixture of ethyl 2-[(14R*,17S)-11-chloro-4,19-difluoro-14-{5-[2-(3-fluoroazetidin-1-yl)ethyl]-2-oxo-4-(trifluoromethyl)pyridin-1-yl}-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (80 mg, 0.10 mmol, 1.00 equiv.) and H₂O (1 mL) in THF (3 mL) was added LiOH·H₂O (8 mg, 0.21 mmol, 2.00 equiv.) at room temperature under nitrogen atmosphere. The resulting mixture was stirred for 3 h at room temperature under nitrogen atmosphere. The mixture was acidified to pH 5 with 1 M HCl (aq.). The resulting mixture was concentrated under reduced pressure. The crude product (120 mg) was purified by Prep-HPLC (Column: XSelect CSH Prep C18 OBD Column, 30*150 mm, 5 μm; Mobile Phase A: water (0.1% HCOOH), Mobile Phase B: MeCN; Flow rate: 60 mL/min; Gradient: 21% B to 41% B in 10 min; Wavelength: 254 nm/220 nm; RT1 (min) 9.63) to afford [(14R*,17S)-11-chloro-4,19-difluoro-14-{5-[2-(3-fluoroazetidin-1-yl)ethyl]-2-oxo-4-(trifluoromethyl)pyridin-1-yl}-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid (54.6 mg, 70.8%) as a white solid. LC-MS: (ES+H, m/z) [M+H]⁺=734.10. ¹H NMR (400 MHz, DMSO-d₆) δ 12.47 (s, 1H), 8.94 (d, J=7.2 Hz, 1H), 7.78 (dd, J=9.0, 4.8 Hz, 1H), 7.61 (t, J=2.0 Hz, 1H), 7.36-7.27 (m, 2H), 7.18 (s, 1H), 7.03 (dd, J=6.9, 2.2 Hz, 1H), 6.79 (s, 1H), 6.43-6.36 (m, 2H), 5.90 (t, J=1.9 Hz, 1H), 5.46 (dt, J=8.8, 6.4 Hz, 1H), 5.00-4.80 (m, 1H), 3.30-3.16 (m, 2H), 2.86 (ddt, J=24.2, 8.4, 4.4 Hz, 2H), 2.75-2.57 (m, 2H), 2.46-2.30 (m, 4H), 2.29 (d, J=1.8 Hz, 3H), 1.85 (d, J=2.3 Hz, 3H). ¹⁹F NMR (377 MHz, DMSO-d₆) δ −62.53, −118.42, −124.95, −176.94.

### Example 35

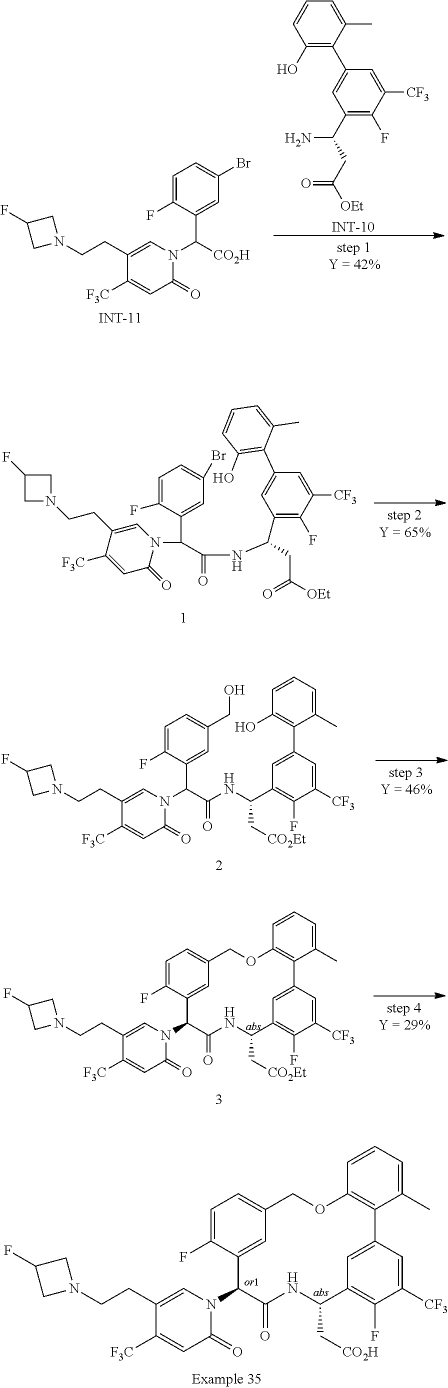

### Step 1: Preparation of ethyl (3S)-3-(2-(5-bromo-2-fluorophenyl)-2-(5-(2-(3-fluoroazetidin-1-yl)ethyl)-2-oxo-4-(trifluoromethyl)pyridin-1(2H)-yl)acetamido)-3-(4-fluoro-2′-hydroxy-6′-methyl-5-(trifluoromethyl)-[1,1′-biphenyl]-3-yl)propanoate

A solution of (5-bromo-2-fluorophenyl)({5-[2-(3-fluoroazetidin-1-yl)ethyl]-2-oxo-4-(trifluoromethyl)pyridin-1-yl})acetic acid (500 mg, 1.01 mmol, 1.00 equiv.) in MeCN (15 mL) was treated with EDCI (290 mg, 1.51 mmol, 1.50 equiv.) and HOBT (177 mg, 1.31 mmol, 1.30 equiv.) at room temperature under nitrogen atmosphere. The resulting mixture was stirred for 1 h and then ethyl (3S)-3-amino-3-[4-fluoro-2′-hydroxy-6′-methyl-5-(trifluoromethyl)-[1,1′-biphenyl]-3-yl]propanoate (389 mg, 1.01 mmol, 1.00 equiv.) and Et₃N (1021 mg, 10.10 mmol, 10.00 equiv.) was added at room temperature. The resulting mixture was stirred overnight at room temperature and then was concentrated and diluted with EtOAc (150 mL). The combined organic layers were washed with brine (3×50 mL), dried over anhydrous Na₂SO₄, filtered, and concentrated. The residue was purified by reversed-phase flash chromatography (C18 gel; mobile phase, MeCN in water (10 mmol/L NH₄HCO₃), 10% to 90% gradient in 40 min; detector, UV 254/220 nm) to provide ethyl (3S)-3-(2-(5-bromo-2-fluorophenyl)-2-(5-(2-(3-fluoroazetidin-1-yl)ethyl)-2-oxo-4-(trifluoromethyl)pyridin-1(2H)-yl)acetamido)-3-(4-fluoro-2′-hydroxy-6′-methyl-5-(trifluoromethyl)-[1,1′-biphenyl]-3-yl)propanoate (390 mg, 42.5%, mixture) as a white solid. LC-MS: (ES+H, m/z) [M+H]⁺=862.0. ¹H NMR (400 MHz, CD₃OD) δ 7.74-7.66 (m, 1H), 7.63-7.58 (m, 1H), 7.54 (dd, J=6.9, 2.1 Hz, 1H), 7.47 (d, J=6.6 Hz, 1H), 7.42 (dd, J=6.6, 2.1 Hz, 1H), 7.37 (d, J=3.5 Hz, 1H), 7.31-7.25 (m, 1H), 7.23-7.11 (m, 1H), 7.08 (td, J=7.9, 2.7 Hz, 1H), 6.94-6.85 (m, 1H), 6.79-6.70 (m, 2H), 5.81-5.70 (m, 1H), 5.06-4.91 (m, 1H), 4.17-4.06 (m, 2H), 3.55-3.39 (m, 2H), 3.11-2.96 (m, 4H), 2.64-2.44 (m, 4H), 1.98 (d, J=20.2 Hz, 3H), 1.16 (td, J=7.1, 2.2 Hz, 3H).

### Step 2: Preparation of ethyl (3S)-3-(4-fluoro-2′-hydroxy-6′-methyl-5-(trifluoromethyl)-[1,1′-biphenyl]-3-yl)-3-(2-(2-fluoro-5-(hydroxymethyl)phenyl)-2-(5-(2-(3-fluoroazetidin-1-yl)ethyl)-2-oxo-4-(trifluoromethyl)pyridin-1(2H)-yl)acetamido)propanoate

To a stirred solution of ethyl (3S)-3-[2-(5-bromo-2-fluorophenyl)-2-{5-[2-(3-fluoroazetidin-1-yl)ethyl]-2-oxo-4-(trifluoromethyl)pyridin-1-yl}acetamido]-3-[4-fluoro-2′-hydroxy-6′-methyl-5-(trifluoromethyl)-[1,1′-biphenyl]-3-yl]propanoate (390 mg, 0.45 mmol, 1.00 equiv., mixture) and (tributylstannyl)methanol (290 mg, 0.90 mmol, 2.00 equiv.) in 1,4-dioxane (8.00 mL) was added dicyclohexyl[2′,4′,6′-tris(propan-2-yl)-[1,1′-biphenyl]-2-yl]phosphane; {2′-amino-[1,1′-biphenyl]-2-yl}(chloro)palladium (35 mg, 0.04 mmol, 0.10 equiv.) at room temperature under nitrogen atmosphere. The resulting mixture was stirred for 5 h at 90° C. under nitrogen atmosphere and then cooled to room temperature. The resulting mixture was concentrated under reduced pressure. The residue was purified by reversed-phase flash chromatography (C18 gel; mobile phase, MeCN in water (10 mmol/L NH₄HCO₃), 10% to 90% gradient in 40 min; detector, UV 254/220 nm) to provide ethyl (3S)-3-(4-fluoro-2′-hydroxy-6′-methyl-5-(trifluoromethyl)-[1,1′-biphenyl]-3-yl)-3-(2-(2-fluoro-5-(hydroxymethyl)phenyl)-2-(5-(2-(3-fluoroazetidin-1-yl)ethyl)-2-oxo-4-(trifluoromethyl)pyridin-1(2H)-yl)acetamido)propanoate (250 mg, 64.5%, mixture) as a white solid. LC-MS: (ES+H, m/z) [M+H]⁺=814.3.

### Step 3: Preparation of ethyl 2-[(18S)-13,20-difluoro-15-{5-[2-(3-fluoroazetidin-1-yl)ethyl]-2-oxo-4-(trifluoromethyl)pyridin-1-yl}-3-methyl-16-oxo-21-(trifluoromethyl)-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetate

To a stirred solution of ethyl (3S)-3-[4-fluoro-2′-hydroxy-6′-methyl-5-(trifluoromethyl)-[1,1′-biphenyl]-3-yl]-3-{2-[2-fluoro-5-(hydroxymethyl)phenyl]-2-{5-[2-(3-fluoroazetidin-1-yl)ethyl]-2-oxo-4-(trifluoromethyl)pyridin-1-yl}acetamido}propanoate (230 mg, 0.28 mmol, 1.00 equiv., mixture) and 2-(tributyl-lambda5-phosphanylidene)acetonitrile (341 mg, 1.41 mmol, 5.00 equiv.) in toluene (5 mL) at room temperature under nitrogen atmosphere. The resulting mixture was stirred overnight at 100° C. and then cooled to room temperature and concentrated. The resulting mixture was diluted with EtOAc (100 mL) and washed with brine (3×30 mL), dried over anhydrous Na₂SO₄, filtered, and concentrated. The residue was purified by reversed-phase flash chromatography (C18 gel; mobile phase, MeCN in water (10 mmol/L NH₄HCO₃), 10% to 90% gradient in 40 min; detector, UV 254/220 nm) to provide ethyl 2-((9S)-1⁴,5⁴-difluoro-6-(5-(2-(3-fluoroazetidin-1-yl)ethyl)-2-oxo-4-(trifluoromethyl)pyridin-1(2H)-yl)-2⁶-methyl-7-oxo-1⁵-(trifluoromethyl)-3-oxa-8-aza-1,5(1,3),2(1,2)-tribenzenacyclononaphane-9-yl)acetate (110 mg, 46.4%) as a light yellow solid. LC-MS: (ES+H, m/z) [M+H]⁺=796.3.

### Step 4: Preparation of [(15R*,18S)-13,20-difluoro-15-{5-[2-(3-fluoroazetidin-1-yl)ethyl]-2-oxo-4-(trifluoromethyl)pyridin-1-yl}-3-methyl-16-oxo-21-(trifluoromethyl)-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetic acid

To a stirred solution of ethyl 2-[(18S)-13,20-difluoro-15-{5-[2-(3-fluoroazetidin-1-yl)ethyl]-2-oxo-4-(trifluoromethyl)pyridin-1-yl}-3-methyl-16-oxo-21-(trifluoromethyl)-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetate (110 mg, 0.13 mmol, 1.00 equiv.) in THF (2.00 mL) and H₂O (0.50 mL) was added LiOH·H₂O (29 mg, 0.69 mmol, 5.00 equiv.) at room temperature under nitrogen atmosphere. The resulting mixture was stirred for 2 h at room and then neutralized to pH 7 with 1 M HCl (aq.), and concentrated. The residue was dissolved in THF (2.00 mL) and purified by Prep-HPLC (Column: Xselect CSH C18 OBD Column 30*150 mm, 5 μm; Mobile Phase A: water (0.1% HCO₂H), Mobile Phase B: MeCN; Flow rate: 60 mL/min; Gradient: 27% B to 41% B in 8 min; Wavelength: 254 nm/220 nm; RT1 (min) 9.03) to afford [(15R*,18S)-13,20-difluoro-15-{5-[2-(3-fluoroazetidin-1-yl)ethyl]-2-oxo-4-(trifluoromethyl)pyridin-1-yl}-3-methyl-16-oxo-21-(trifluoromethyl)-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetic acid (31.5 mg, 29.1%) as a white solid. LC-MS: (ES+H, m/z) [M+H]⁺=768.15. ¹H NMR (300 MHz, CD₃OD) δ 7.53-7.50 (m, 1H), 7.42-7.40 (m, 1H), 7.30-7.11 (m, 7H), 6.91 (d, J=7.3 Hz, 2H), 5.74 (t, J=7.0 Hz, 1H), 5.36 (d, J=13.2 Hz, 1H), 5.20-5.00 (m, 2H), 3.73-3.62 (m, 2H), 3.39-3.34 (m, 1H), 3.30-3.26 (m, 1H), 2.80-2.68 (m, 4H), 2.53-2.47 (m, 2H), 2.02 (s, 3H). ¹⁹F NMR (282 MHz, CD₃OD) δ −62.46, −64.92, −118.20, −124.79, −180.58.

### Example 36

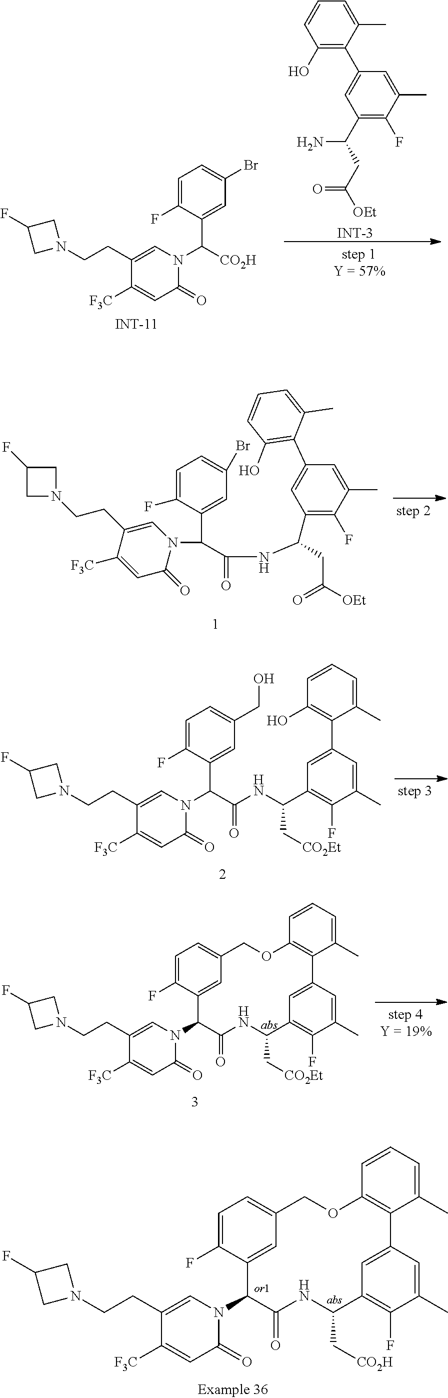

### Step 1: Preparation of ethyl (3S)-3-[2-(5-bromo-2-fluorophenyl)-2-{5-[2-(3-fluoroazetidin-1-yl)ethyl]-2-oxo-4-(trifluoromethyl)pyridin-1-yl}acetamido]-3-{4-fluoro-2′-hydroxy-5,6′-dimethyl-[1,1′-biphenyl]-3-yl}propanoate

To a stirred mixture of (5-bromo-2-fluorophenyl)({5-[2-(3-fluoroazetidin-1-yl)ethyl]-2-oxo-4-(trifluoromethyl)pyridin-1-yl})acetic acid (300 mg, 0.60 mmol, 1.00 equiv.) and HOBT (106 mg, 0.79 mmol, 1.30 equiv.) in CH₃CN (5 mL) was added EDCI (174 mg, 0.91 mmol, 1.50 equiv.) in portions at room temperature under nitrogen atmosphere. The resulting mixture was stirred for 1 h at room temperature and then ethyl (3S)-3-amino-3-{4-fluoro-2′-hydroxy-5,6′-dimethyl-[1,1′-biphenyl]-3-yl}propanoate (200 mg, 0.60 mmol, 1.00 equiv.), Et₃N (245 mg, 2.42 mmol, 4.00 equiv.) in CH₃CN (3 mL) was added dropwise over 1 min at room temperature. The resulting mixture was stirred overnight at 40° C., and then diluted with EtOAc (150 mL), washed with water (2×20 mL), dried over anhydrous Na₂SO₄, filtered, and concentrated. The residue was purified by reversed-phase flash chromatography (C18 gel; mobile phase, MeCN in water (0.1% NH₃·H₂O), 0% to 70% gradient in 40 min; detector, UV 220 nm) to provide ethyl (3S)-3-[2-(5-bromo-2-fluorophenyl)-2-{5-[2-(3-fluoroazetidin-1-yl)ethyl]-2-oxo-4-(trifluoromethyl)pyridin-1-yl}acetamido]-3-{4-fluoro-2′-hydroxy-5,6′-dimethyl-[1,1′-biphenyl]-3-yl}propanoate (280 mg, 57.1%, mixture) as a white solid. LC-MS: (ES+H, m/z) [M+H]⁺=808.0. ¹H NMR (300 MHz, CD₃OD) δ 7.70-7.59 (m, 1H), 7.40-7.08 (m, 3H), 7.06-6.92 (m, 4H), 6.86-6.67 (m, 3H), 5.72 (q, J=5.5, 3.7 Hz, 1H), 5.06-4.80 (m, 1H), 4.16-4.06 (m, 2H), 3.59-3.39 (m, 2H), 3.03-2.88 (m, 3H), 2.86 (d, J=7.2 Hz, 1H), 2.65-2.43 (m, 4H), 2.30 (dd, J=7.7, 2.1 Hz, 3H), 2.02-1.90 (m, 3H), 1.16 (td, J=7.1, 2.0 Hz, 3H).

### Step 2: Preparation of ethyl (3S)-3-{4-fluoro-2′-hydroxy-5,6′-dimethyl-[1,1′-biphenyl]-3-yl}-3-{2-[2-fluoro-5-(hydroxymethyl)phenyl]-2-{5-[2-(3-fluoroazetidin-1-yl)ethyl]-2-oxo-4-(trifluoromethyl)pyridin-1-yl}acetamido}propanoate

To a stirred solution of ethyl (3S)-3-[2-(5-bromo-2-fluorophenyl)-2-{5-[2-(3-fluoroazetidin-1-yl)ethyl]-2-oxo-4-(trifluoromethyl)pyridin-1-yl}acetamido]-3-{4-fluoro-2′-hydroxy-5,6′-dimethyl-[1,1′-biphenyl]-3-yl}propanoate (280 mg, 0.34 mmol, 1.00 equiv., mixture) and (tributylstannyl)methanol (222 mg, 0.69 mmol, 2.00 equiv.) in dioxane (10 mL) was added dicyclohexyl[2′,4′,6′-tris(propan-2-yl)-[1,1′-biphenyl]-2-yl]phosphane; {2′-amino-[1,1′-biphenyl]-2-yl}(chloro)palladium (27 mg, 0.03 mmol, 0.10 equiv.) in portions at room temperature under nitrogen atmosphere. The resulting mixture was stirred for 5 h at 90° C. under nitrogen atmosphere and then cooled to room temperature, diluted with EtOAc (100 mL), washed with water (2×20 mL), dried over anhydrous Na₂SO₄, filtered, and concentrated. The residue was purified by reversed-phase flash chromatography (C18 gel; mobile phase, MeCN in water (0.1% NH₃·H₂O), 10% to 70% gradient in 40 min; detector, UV 220 nm) to provide ethyl (3S)-3-{4-fluoro-2′-hydroxy-5,6′-dimethyl-[1,1′-biphenyl]-3-yl}-3-{2-[2-fluoro-5-(hydroxymethyl)phenyl]-2-{5-[2-(3-fluoroazetidin-1-yl)ethyl]-2-oxo-4-(trifluoromethyl)pyridin-1-yl}acetamido}propanoate (180 mg, crude, mixture) as a brown solid. LC-MS: (ES+H, m/z) [M+H]⁺=760.3.

### Step 3: Preparation of ethyl 2-[(18S)-13,20-difluoro-15-{5-[2-(3-fluoroazetidin-1-yl)ethyl]-2-oxo-4-(trifluoromethyl)pyridin-1-yl}-3,21-dimethyl-16-oxo-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetate

A solution of ethyl (3S)-3-{4-fluoro-2′-hydroxy-5,6′-dimethyl-[1,1′-biphenyl]-3-yl}-3-{2-[2-fluoro-5-(hydroxymethyl)phenyl]-2-{5-[2-(3-fluoroazetidin-1-yl)ethyl]-2-oxo-4-(trifluoromethyl)pyridin-1-yl}acetamido}propanoate (170 mg, crude) and 2-(tributyl-lambda5-phosphanylidene)acetonitrile (270 mg, 1.12 mmol, 5.00 equiv.) in PhCH₃ (5 mL) was stirred overnight at 100° C. under nitrogen atmosphere and then cooled to room temperature. The resulting mixture was diluted with EtOAc (100 mL), and washed with water (2×20 mL), dried over anhydrous Na₂SO₄, filtered, and concentrated. The residue was purified by reversed-phase flash chromatography (C18 gel; mobile phase, MeCN in water (0.1% NH₃·H₂O), 0% to 70% gradient in 40 min; detector, UV 220 nm) to provide ethyl 2-[(18S)-13,20-difluoro-15-{5-[2-(3-fluoroazetidin-1-yl)ethyl]-2-oxo-4-(trifluoromethyl)pyridin-1-yl}-3,21-dimethyl-16-oxo-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetate (65 mg, crude) as a brown solid. LC-MS: (ES+H, m/z) [M+H]⁺=742.2.

### Step 4: Preparation of [(15R*,18S)-13,20-difluoro-15-{5-[2-(3-fluoroazetidin-1-yl)ethyl]-2-oxo-4-(trifluoromethyl)pyridin-1-yl}-3,21-dimethyl-16-oxo-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetic acid

To a stirred solution of ethyl 2-[(18S)-13,20-difluoro-15-{5-[2-(3-fluoroazetidin-1-yl)ethyl]-2-oxo-4-(trifluoromethyl)pyridin-1-yl}-3,21-dimethyl-16-oxo-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetate (60 mg, 0.08 mmol, 1.00 equiv.) and LiOH·H₂O (17 mg, 0.40 mmol, 5.00 equiv.) in THF (1.5 mL) was added H₂O (0.3 mL) dropwise at room temperature under nitrogen atmosphere. The resulting mixture was stirred for 2 h at room temperature under nitrogen atmosphere and then neutralized to pH 7 with 1 M HCl (aq.) and concentrated. The residue was purified by reverse flash chromatography (Column: Xselect CSH C18 OBD Column 30*150 mm, 5 μm; Mobile Phase A: water (0.10% HCOOH), Mobile Phase B: MeOH; Flow rate: 60 mL/min; Gradient: 44% B to 54% B in 8 min; Wavelength: 254 nm/220 nm; RT1 (min) 7.37) To afford [(15R*,18S)-13,20-difluoro-15-{5-[2-(3-fluoroazetidin-1-yl)ethyl]-2-oxo-4-(trifluoromethyl)pyridin-1-yl}-3,21-dimethyl-16-oxo-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetic acid (11.5 mg, 19.3%) as a white solid. LC-MS: (ES+H, m/z) [M+H]⁺=714.15. ¹H NMR (400 MHz, CD₃OD) δ 7.49-7.46 (m, 1H), 7.35-7.33 (m, 1H), 7.21-7.08 (m, 5H), 6.95 (dd, J=6.8, 2.1 Hz, 1H), 6.87-6.81 (m, 2H), 6.69 (dd, J=6.8, 2.2 Hz, 1H), 5.73 (dd, J=8.4, 5.7 Hz, 1H), 5.31 (d, J=13.2 Hz, 1H), 5.05-4.80 (m, 2H), 3.61 (t, J=8.6 Hz, 2H), 3.31-3.20 (m, 2H), 2.75-2.60 (m, 4H), 2.49-2.44 (m, 2H), 2.31 (s, 3H), 1.98 (s, 3H). ¹⁹F NMR (377 MHz, CD₃OD) δ −64.92, −118.24, −128.23, −180.52.

### Example 37

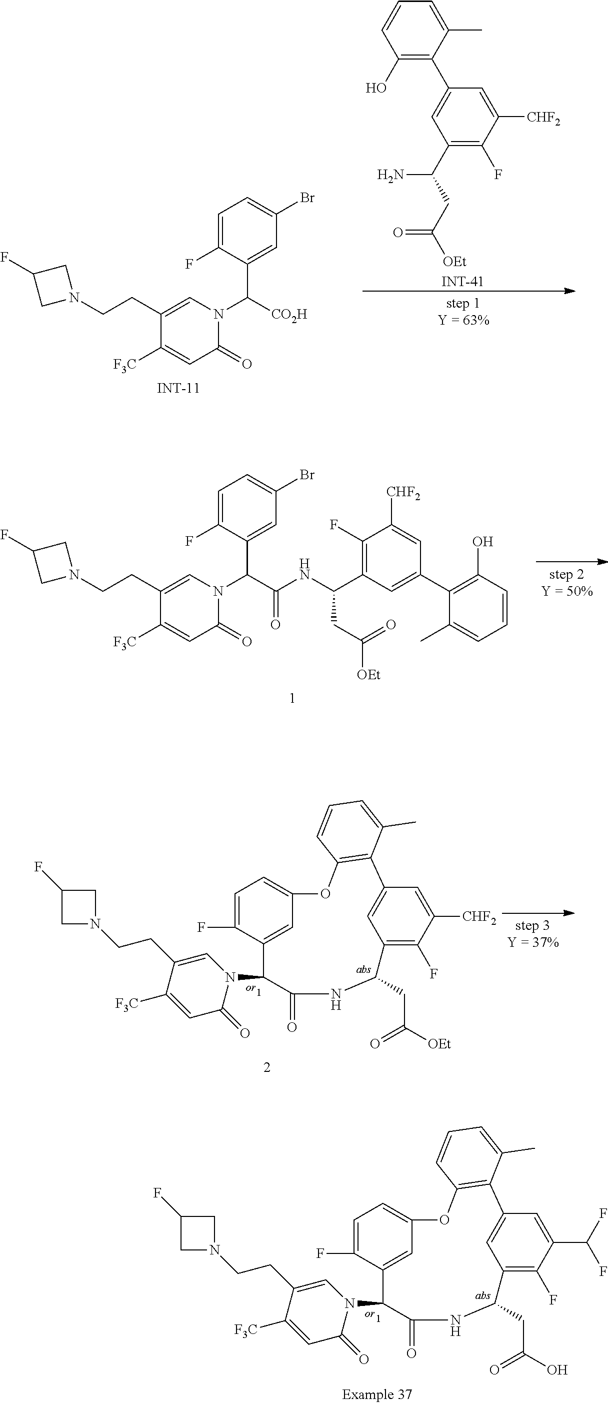

### Step 1: Preparation of ethyl (3S)-3-(2-(5-bromo-2-fluorophenyl)-2-(5-(2-(3-fluoroazetidin-1-yl)ethyl)-2-oxo-4-(trifluoromethyl)pyridin-1(2H)-yl)acetamido)-3-(5-(difluoromethyl)-4-fluoro-2′-hydroxy-6′-methyl-[1,1′-biphenyl]-3-yl)propanoate

A mixture of ethyl (S)-3-amino-3-(5-(difluoromethyl)-4-fluoro-2′-hydroxy-6′-methyl-[1,1′-biphenyl]-3-yl)propanoate hydrochloride (190 mg, 0.47 mmol, 1.00 equiv.) and NEt₃ (286 mg, 2.82 mmol, 6.00 equiv.) in MeCN (5 mL) was stirred for 30 min at 30° C. under nitrogen atmosphere. The resulting mixture was concentrated under reduced pressure and then redissolved in MeCN (5 mL). To a stirred solution of 2-(5-bromo-2-fluorophenyl)-2-(5-(2-(3-fluoroazetidin-1-yl)ethyl)-2-oxo-4-(trifluoromethyl)pyridin-1(2H)-yl)acetic acid (232 mg, 0.47 mmol, 1.00 equiv.) in MeCN (5 mL) was added EDCI (135 mg, 0.71 mmol, 1.50 equiv.) and HOBT (83 mg, 0.61 mmol, 1.30 equiv.). The resulting mixture was stirred for 1 h at 30° C. under nitrogen atmosphere and then ethyl (S)-3-amino-3-(5-(difluoromethyl)-4-fluoro-2′-hydroxy-6′-methyl-[1,1′-biphenyl]-3-yl)propanoate in MeCN (5 mL) was added dropwise at room temperature. The resulting mixture was stirred overnight at 40° C. The mixture was cooled to room temperature and concentrated. The residue was purified by reversed-phase flash chromatography (C18 gel; mobile phase, MeCN in water (0.1% NH₃·H₂O), 45% to 70% gradient in 30 min; detector, UV 220 nm) to provide ethyl (3S)-3-(2-(5-bromo-2-fluorophenyl)-2-(5-(2-(3-fluoroazetidin-1-yl)ethyl)-2-oxo-4-(trifluoromethyl)pyridin-1(2H)-yl)acetamido)-3-(5-(difluoromethyl)-4-fluoro-2′-hydroxy-6′-methyl-[1,1′-biphenyl]-3-yl)propanoate (250 mg, 62.9%, mixture) as a yellow solid. LC-MS: (ES+H, m/z) [M+H]⁺=844.2.

### Step 2: Preparation of ethyl 2-[(14R*,17S)-20-(difluoromethyl)-12,19-difluoro-14-{5-[2-(3-fluoroazetidin-1-yl)ethyl]-2-oxo-4-(trifluoromethyl)pyridin-1-yl}-3-methyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate

To a stirred mixture of ethyl (3S)-3-(2-(5-bromo-2-fluorophenyl)-2-(5-(2-(3-fluoroazetidin-1-yl)ethyl)-2-oxo-4-(trifluoromethyl)pyridin-1(2H)-yl)acetamido)-3-(5-(difluoromethyl)-4-fluoro-2′-hydroxy-6′-methyl-[1,1′-biphenyl]-3-yl)propanoate (220 mg, 0.26 mmol, 1.00 equiv.) and CuI (25 mg, 0.13 mmol, 0.50 equiv.) in 1,4-dioxane (15 mL) were added K₂CO₃ (72 mg, 0.52 mmol, 2.00 equiv.) and (1R,2R)—N1,N2-dimethylcyclohexane-1,2-diamine (37 mg, 0.26 mmol, 1.00 equiv.). The resulting mixture was stirred overnight at 100° C. under nitrogen atmosphere and then cooled to room temperature and concentrated. The residue was purified by reversed-phase flash chromatography (C18 gel; mobile phase, MeCN in water (0.1% NH₃·H₂O), 45% to 70% gradient in 30 min; detector, UV 220 nm) to provide ethyl 2-[(14R*,17S)-20-(difluoromethyl)-12,19-difluoro-14-{5-[2-(3-fluoroazetidin-1-yl)ethyl]-2-oxo-4-(trifluoromethyl)pyridin-1-yl}-3-methyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (100 mg, 50.2%) as a yellow solid. LC-MS: (ES+H, m/z) [M+H]⁺=764.3.

### Step 3: Preparation of [(14R*,17S)-20-(difluoromethyl)-12,19-difluoro-14-{5-[2-(3-fluoroazetidin-1-yl)ethyl]-2-oxo-4-(trifluoromethyl)pyridin-1-yl}-3-methyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid

A mixture of ethyl 2-[(14R*,17S)-20-(difluoromethyl)-12,19-difluoro-14-{5-[2-(3-fluoroazetidin-1-yl)ethyl]-2-oxo-4-(trifluoromethyl)pyridin-1-yl}-3-methyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (90 mg, 0.12 mmol, 1.00 equiv.) and LiOH·H₂O (20 mg, 0.47 mmol, 4.00 equiv.) in THF (2 mL) and H₂O (0.5 mL) was stirred for 2 h at room temperature under nitrogen atmosphere. The mixture was then acidified to pH 5 with 3 M HCl (aq.) and concentrated. The residue was purified by Prep-HPLC (Column: XBridge Shield RP18 OBD Column, 30*150 mm, 5 μm; Mobile Phase A: water (10 mmol/L NH₄HCO₃), Mobile Phase B: MeCN; Flow rate: 60 mL/min; Gradient: 25% B to 45% B in 10 min; Wavelength: 254 nm/220 nm; RT1 (min) 9.1) to afford [(14R*,17S)-20-(difluoromethyl)-12,19-difluoro-14-{5-[2-(3-fluoroazetidin-1-yl)ethyl]-2-oxo-4-(trifluoromethyl)pyridin-1-yl}-3-methyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid (32.6 mg, 36.6%) as a white solid. LC-MS: (ES+H, m/z) [M+H]⁺=736.10. ¹H NMR (400 MHz, CD₃OD) δ 7.61-7.54 (m, 1H), 7.49 (d, J=8.0 Hz, 1H), 7.42-7.30 (m, 3H), 7.22-7.18 (m, 2H), 7.07 ((t, J=39.2 Hz, 1H), 6.89-6.82 (m, 3H), 6.04 (dd, J=6.1, 2.9 Hz, 1H), 5.69 (t, J=7.5 Hz, 1H), 5.07-4.89 (m, 1H), 3.58 (m, 2H), 3.28-3.08 (m, 2H), 2.88-2.58 (m, 4H), 2.56-2.39 (m, 2H), 2.00 (s, 3H).

### Example 38

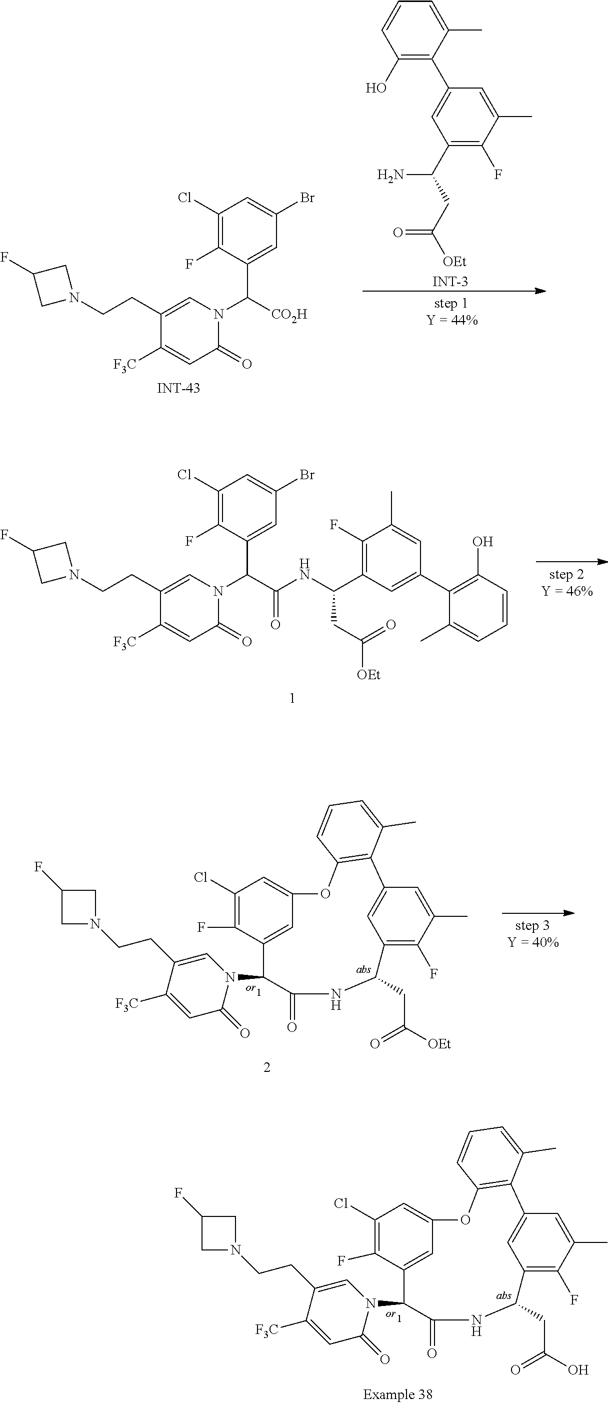

### Step 1: Preparation of ethyl (3S)-3-[2-(5-bromo-3-chloro-2-fluorophenyl)-2-{5-[2-(3-fluoroazetidin-1-yl)ethyl]-2-oxo-4-(trifluoromethyl)pyridin-1-yl}acetamido]-3-{4-fluoro-2′-hydroxy-5,6′-dimethyl-[1,1′-biphenyl]-3-yl}propanoate

A solution of ethyl (3S)-3-amino-3-{4-fluoro-2′-hydroxy-5,6′-dimethyl-[1,1′-biphenyl]-3-yl}propanoate hydrochloride (139 mg, 0.37 mmol, 1.00 equiv.) and Et₃N (382 mg, 3.78 mmol, 10.00 equiv.) in CH₃CN (1 mL) was stirred for 30 min at 40° C. under nitrogen atmosphere. In a separate vial, a mixture of (5-bromo-3-chloro-2-fluorophenyl)({5-[2-(3-fluoroazetidin-1-yl)ethyl]-2-oxo-4-(trifluoromethyl)pyridin-1-yl})acetic acid (200 mg, 0.37 mmol, 1.00 equiv.) and HOBT (66 mg, 0.49 mmol, 1.30 equiv.) in CH₃CN (1 mL) was treated with EDCI (108 mg, 0.56 mmol, 1.50 equiv.) at room temperature under nitrogen atmosphere. The resulting mixture was stirred for 1 h at 40° C. under nitrogen atmosphere and then the previously made solution of ethyl (3S)-3-amino-3-{4-fluoro-2′-hydroxy-5,6′-dimethyl-[1,1′-biphenyl]-3-yl}propanoate hydrochloride in MeCN was added dropwise at 0° C. The resulting mixture was stirred overnight at 40° C., and then concentrated. The residue was purified by reversed-phase flash chromatography (C18 gel; mobile phase, CH₃CN in water (0.1% NH₃·H₂O), 20% to 100% gradient in 30 min; detector, UV 220 nm) to provide ethyl (3S)-3-[2-(5-bromo-3-chloro-2-fluorophenyl)-2-{5-[2-(3-fluoroazetidin-1-yl)ethyl]-2-oxo-4-(trifluoromethyl)pyridin-1-yl}acetamido]-3-{4-fluoro-2′-hydroxy-5,6′-dimethyl-[1,1′-biphenyl]-3-yl}propanoate (140 mg, 44.0%, mixture) as a light yellow solid. LC-MS: (ES−H, m/z) [M−H]⁻=842.1.

### Step 2: Preparation of ethyl 2-[(14S,17S)-11-chloro-12,19-difluoro-14-{5-[2-(3-fluoroazetidin-1-yl)ethyl]-2-oxo-4-(trifluoromethyl)pyridin-1-yl}-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate

To a stirred solution of ethyl (3S)-3-[2-(5-bromo-3-chloro-2-fluorophenyl)-2-{5-[2-(3-fluoroazetidin-1-yl)ethyl]-2-oxo-4-(trifluoromethyl)pyridin-1-yl}acetamido]-3-{4-fluoro-2′-hydroxy-5,6′-dimethyl-[1,1′-biphenyl]-3-yl}propanoate (120 mg, 0.14 mmol, 1.00 equiv., mixture) and (1R,2R)—N1,N2-dimethylcyclohexane-1,2-diamine (24 mg, 0.17 mmol, 1.20 equiv.) in dioxane (3 mL) were added CuI (8 mg, 0.043 mmol, 0.30 equiv.) and K₂CO₃ (39 mg, 0.28 mmol, 2.00 equiv.) at room temperature under nitrogen atmosphere. The resulting mixture was stirred overnight at 100° C., and then cooled to room temperature and filtered. The filter cake was washed with EtOAc (5×20 mL) and the combined filtrate was concentrated under reduced pressure. The residue was purified by reversed-phase flash chromatography (C18 gel; mobile phase, CH₃CN in water (0.1% NH₃·H₂O), 40% to 70% gradient in 30 min; detector, UV 220 nm) to provide ethyl 2-[(14S,17S)-11-chloro-12,19-difluoro-14-{5-[2-(3-fluoroazetidin-1-yl)ethyl]-2-oxo-4-(trifluoromethyl)pyridin-1-yl}-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (50 mg, 46.1%) as a white solid. LC-MS: (ES+H, m/z) [M+H]⁺=762.2.

### Step 3: Preparation of [(14S,17S)-11-chloro-12,19-difluoro-14-{5-[2-(3-fluoroazetidin-1-yl)ethyl]-2-oxo-4-(trifluoromethyl)pyridin-1-yl}-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid

A stirred solution of ethyl 2-[(14S,17S)-11-chloro-12,19-difluoro-14-{5-[2-(3-fluoroazetidin-1-yl)ethyl]-2-oxo-4-(trifluoromethyl)pyridin-1-yl}-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (50 mg, 0.066 mmol, 1.00 equiv.) in THF (2 mL) was added LiOH·H₂O (1 mL, 1.98 mmol, 30.00 equiv., 2 M in water) at room temperature under nitrogen atmosphere. The resulting mixture was stirred for 3 h at room temperature and then acidified to pH 3 with 3 M HCl (aq.), and concentrated. under reduced pressure. The crude product (200 mg) was purified by Prep-HPLC (Column: XSelect CSH Prep C18 OBD Column, 30*150 mm, 5 μm; Mobile Phase A: water (0.1% HCOOH), Mobile Phase B: CH₃CN; Flow rate: 60 mL/min; Gradient: 21% B to 41% B in 10 min; Wavelength: 254 nm/220 nm; RT1 (min) 9.38) to afford [(14S,17S)-11-chloro-12,19-difluoro-14-{5-[2-(3-fluoroazetidin-1-yl)ethyl]-2-oxo-4-(trifluoromethyl)pyridin-1-yl}-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid (20 mg, 40.6%) as a white solid. LC-MS: (ES+H, m/z) [M+H]⁺=734.15. ¹H NMR (300 MHz, DMSO-d₆) δ 13.7-11.5 (m, 1H), 9.13 (d, J=7.4 Hz, 1H), 7.86-7.83 (m, 1H), 7.73 (d, J=8.0 Hz, 1H), 7.41 (t, J=7.8 Hz, 1H), 7.31-7.19 (m, 2H), 7.05-7.03 (m, 1H), 6.83 (s, 1H), 6.72 (s, 1H), 6.43-6.41 (m, 1H), 5.96-5.94 (m, 1H), 5.49 (q, J=7.5 Hz, 1H), 4.97-4.78 (m, 1H), 3.29-3.25 (m, 2H), 2.97-2.88 (m, 1H), 2.86-2.83 (m, 1H), 2.74-2.60 (m, 2H), 2.48-2.32 (m, 4H), 2.30 (d, J=1.8 Hz, 3H), 1.97 (s, 3H). ¹⁹F NMR (282 MHz, DMSO-d₆) δ −62.608, −123.384, −125.580, −177.079.

### Example 39

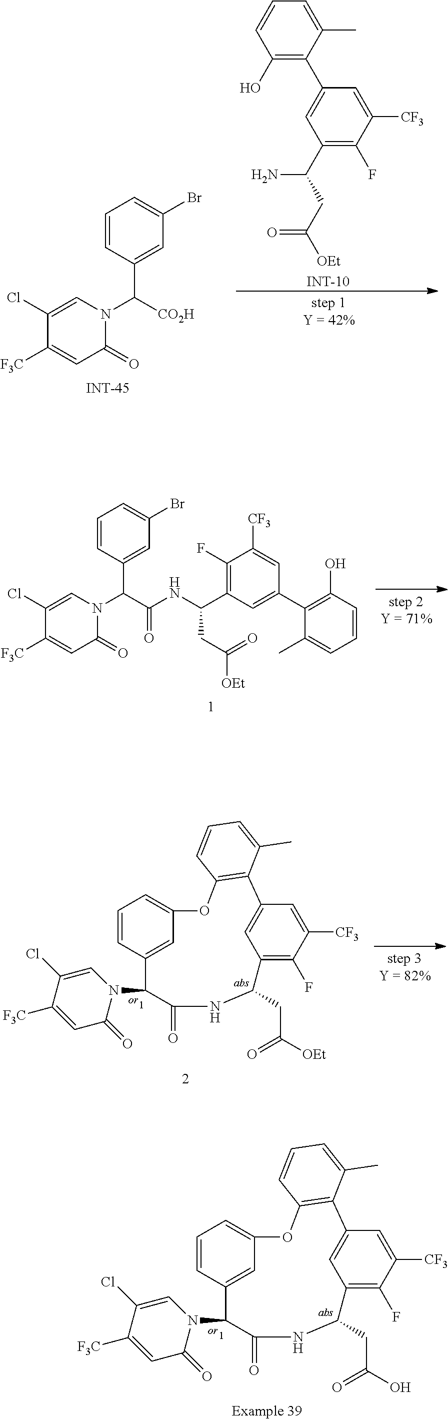

### Step 1: Preparation of ethyl (3S)-3-[(2R*)-2-(3-bromophenyl)-2-[5-chloro-2-oxo-4-(trifluoromethyl)pyridin-1-yl]acetamido]-3-[4-fluoro-2′-hydroxy-6′-methyl-5-(trifluoromethyl)-[1,1′-biphenyl]-3-yl]propanoate

A solution of ethyl (3S)-3-amino-3-[4-fluoro-2′-hydroxy-6′-methyl-5-(trifluoromethyl)-[1,1′-biphenyl]-3-yl]propanoate hydrochloride (188 mg, assumed 100% yield, 0.49 mmol, 1.00 equiv.) and triethylamine (296 mg, 2.92 mmol, 6.00 equiv.) in MeCN (2 mL) was stirred for 1 h at room temperature under nitrogen atmosphere. The resulting mixture was concentrated under reduced pressure to afford ethyl (3S)-3-amino-3-[4-fluoro-2′-hydroxy-6′-methyl-5-(trifluoromethyl)-[1,1′-biphenyl]-3-yl]propanoate. In a separate vial (3-bromophenyl)[5-chloro-2-oxo-4-(trifluoromethyl)pyridin-1-yl]acetic acid (200 mg, 0.49 mmol, 1.00 equiv.), HOBT (132 mg, 0.97 mmol, 2.00 equiv.) and EDCI (187 mg, 0.97 mmol, 2.00 equiv.) in MeCN (2 mL) were stirred for 1 h at room temperature under nitrogen atmosphere. To the above mixture was added ethyl (3S)-3-amino-3-[4-fluoro-2′-hydroxy-6′-methyl-4′,5-bis(trifluoromethyl)-[1,1′-biphenyl]-3-yl]propanoate in MeCN (2 mL) dropwise over 5 min at 0° C. The resulting mixture was stirred overnight at 30° C. and then concentrated. The residue was purified by reversed-phase flash chromatography (C18 gel; mobile phase, MeCN in water (0.1% NH₃·H₂O), 40% to 70% gradient in 20 min; detector, UV 220 nm) to provide ethyl (3S)-3-[(2R*)-2-(3-bromophenyl)-2-[5-chloro-2-oxo-4-(trifluoromethyl)pyridin-1-yl]acetamido]-3-[4-fluoro-2′-hydroxy-6′-methyl-5-(trifluoromethyl)-[1,1′-biphenyl]-3-yl]propanoate (160 mg, 42.2%, mixture) as a colorless oil. LC-MS: (ES+H, m/z) [M+H]⁺=776.95.

### Step 2: Preparation of ethyl 2-[(17S)-14-[5-chloro-2-oxo-4-(trifluoromethyl)pyridin-1-yl]-19-fluoro-3-methyl-15-oxo-20-(trifluoromethyl)-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate

A mixture of ethyl (3S)-3-[2-(3-bromophenyl)-2-[5-chloro-2-oxo-4-(trifluoromethyl)pyridin-1-yl]acetamido]-3-[4-fluoro-2′-hydroxy-6′-methyl-5-(trifluoromethyl)-[1,1′-biphenyl]-3-yl]propanoate (110 mg, 0.14 mmol, 1.00 equiv.), CuI (13 mg, 0.07 mmol, 0.50 equiv.), K₂CO₃ (39 mg, 0.28 mmol, 2.00 equiv.) and (1R,2R)—N1,N2-dimethylcyclohexane-1,2-diamine (20 mg, 0.14 mmol, 1.00 equiv.) in 1,4-dioxane (6 mL) was stirred overnight at 100° C. under nitrogen atmosphere. The resulting mixture was concentrated under reduced pressure. The residue was purified by reversed-phase flash chromatography (C18 gel; mobile phase, MeCN in water (0.1% NH₃·H₂O), 40% to 70% gradient in 15 min; detector, UV 220 nm) to provide ethyl 2-[(17S)-14-[5-chloro-2-oxo-4-(trifluoromethyl)pyridin-1-yl]-19-fluoro-3-methyl-15-oxo-20-(trifluoromethyl)-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (70 mg, 71.0%) as a white solid. LC-MS: (ES+H, m/z) [M+H]⁺=697.15. ¹H NMR (300 MHz, CDCl₃) δ 7.50 (s, 1H), 7.43-7.36 (m, 5H), 7.29 (d, J=7.2 Hz, 1H), 7.21-7.09 (m, 2H), 6.93 (s, 1H), 6.68 (s, 2H), 6.39 (d, J=6.6 Hz, 1H), 5.78 (s, 1H), 4.10-3.86 (m, 2H), 2.84 (d, J=10.5 Hz, 2H), 1.99-1.93 (m, 3H), 1.16-1.07 (m, 3H).

### Step 3: Preparation of [(17S)-14-[5-chloro-2-oxo-4-(trifluoromethyl)pyridin-1-yl]-19-fluoro-3-methyl-15-oxo-20-(trifluoromethyl)-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid

A mixture of ethyl 2-[(17S)-14-[5-chloro-2-oxo-4-(trifluoromethyl)pyridin-1-yl]-19-fluoro-3-methyl-15-oxo-20-(trifluoromethyl)-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (12 mg, 0.02 mmol, 1.00 equiv.) and LiOH·H₂O (3 mg, 0.07 mmol, 4.00 equiv.) in THF (2 mL) and H₂O (0.5 mL) was stirred for 3 h at room temperature under nitrogen atmosphere. The mixture was acidified to pH 4 with 1 M HCl (aq.) and then concentrated. The crude product was purified by Prep-HPLC (Column: XSelect CSH Prep C18 OBD Column, 30*150 mm, 5 μm; Mobile Phase A: water (0.1% HCOOH), Mobile Phase B: MeCN; Flow rate: 60 mL/min; Gradient: 59% B to 79% B in 10 min; Wavelength: 254 nm/220 nm; RT1 (min) 8.25) to afford [(17S)-14-[5-chloro-2-oxo-4-(trifluoromethyl)pyridin-1-yl]-19-fluoro-3-methyl-15-oxo-20-(trifluoromethyl)-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid (9.5 mg, 81.8%) as a white solid. LC-MS: (ES+H, m/z) [M+H]⁺=669.05. ¹H NMR (300 MHz, CD₃OD) δ 7.53-7.38 (m, 5H), 7.28-7.12 (m, 3H), 6.99 (s, 1H), 6.85-6.77 (m, 1H), 6.51 (s, 1H), 6.04-6.00 (m, 1H), 5.68 (t, J=7.6 Hz, 1H), 2.96-2.62 (m, 2H), 1.98 (s, 3H). ¹⁹F NMR (282 MHz, CD₃OD) δ −62.55, −66.99, −123.56.

### Example 40

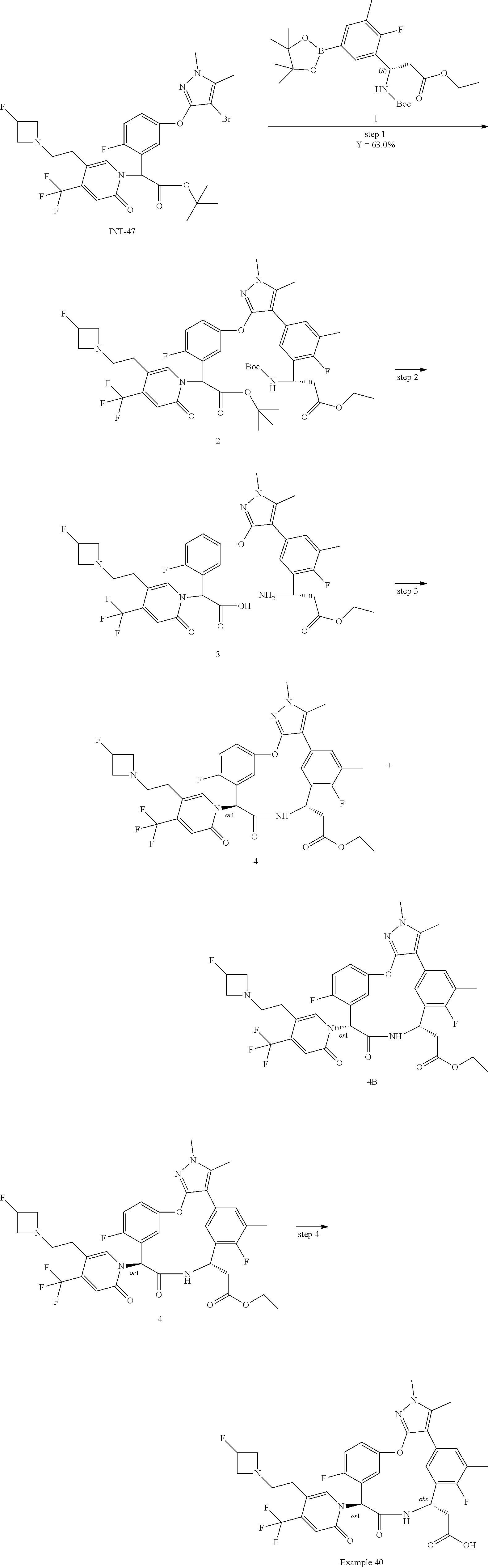

### Step 1: Preparation of ethyl (3S)-3-[5-(3-{3-[2-(tert-butoxy)-1-{5-[2-(3-fluoroazetidin-1-yl)ethyl]-2-oxo-4-(trifluoromethyl)pyridin-1-yl}-2-oxoethyl]-4-fluorophenoxy}-1,5-dimethylpyrazol-4-yl)-2-fluoro-3-methylphenyl]-3-[(tert-butoxycarbonyl)amino]propanoate

To a stirred solution of tert-butyl 2-{5-[(4-bromo-1,5-dimethylpyrazol-3-yl)oxy]-2-fluorophenyl}-2-{5-[2-(3-fluoroazetidin-1-yl)ethyl]-2-oxo-4-(trifluoromethyl)pyridin-1-yl}acetate (350 mg, 0.53 mmol, 1.00 equiv.) and ethyl (3S)-3-[(tert-butoxycarbonyl)amino]-3-[2-fluoro-3-methyl-5-(4,4,5,5-tetramethyl-1,3,2-dioxaborolan-2-yl)phenyl]propanoate (286 mg, 0.63 mmol, 1.20 equiv.) in dioxane (10 mL) and H₂O (1 mL) were added Pd(OAc)₂ (24 mg, 0.10 mmol, 0.20 equiv.), XPhos (25 mg, 0.05 mmol, 0.10 equiv.) and K₂CO₃ (219 mg, 1.58 mmol, 3.00 equiv.) in portions at room temperature. The resulting mixture was stirred for 3 h at 100° C. under nitrogen atmosphere and then cooled to room temperature. The reaction was filtered, and the filter cake was washed with CH₂Cl₂/MeOH=10/1 (3×100 mL) and concentrated. The residue was purified by reversed-phase flash chromatography (C18 gel; mobile phase, MeCN in water (0.1% NH₃·H₂O), 0% to 70% gradient in 40 min; detector, UV 220 nm) to provide ethyl (3S)-3-[5-(3-{3-[2-(tert-butoxy)-1-{5-[2-(3-fluoroazetidin-1-yl)ethyl]-2-oxo-4-(trifluoromethyl)pyridin-1-yl}-2-oxoethyl]-4-fluorophenoxy}-1,5-dimethylpyrazol-4-yl)-2-fluoro-3-methylphenyl]-3-[(tert-butoxycarbonyl)amino]propanoate (mixture, 600 mg, 63.0%) as a white solid. LC-MS: (ES+H, m/z) [M+H]⁺=906.40. ¹H NMR (300 MHz, CD₃OD) δ7.39 (d, J=7.6 Hz, 1H), 7.26-7.16 (m, 3H), 7.14-7.07 (m, 2H), 6.89 (s, 1H), 6.53 (s, 1H), 5.32 (d, J=8.9 Hz, 1H), 5.10-4.86 (m, 1H), 4.18-4.03 (m, 3H), 3.77 (s, 3H), 3.51-3.39 (m, 2H), 3.12-2.98 (m, 2H), 2.72 (t, J=8.2 Hz, 2H), 2.59-2.45 (m, 4H), 2.35 (d, J=2.6 Hz, 3H), 2.25 (d, J=2.0 Hz, 3H), 1.45 (s, 18H), 1.22-1.18 (m, 3H).

### Step 2: Preparation of {5-[(4-{3-[(1S)-1-amino-3-ethoxy-3-oxopropyl]-4-fluoro-5-methylphenyl}-1,5-dimethylpyrazol-3-yl)oxy]-2-fluorophenyl}({5-[2-(3-fluoroazetidin-1-yl)ethyl]-2-oxo-4-(trifluoromethyl)pyridin-1-yl})acetic acid

To a stirred solution of ethyl (3S)-3-[5-(3-{3-[2-(tert-butoxy)-1-{5-[2-(3-fluoroazetidin-1-yl)ethyl]-2-oxo-4-(trifluoromethyl)pyridin-1-yl}-2-oxoethyl]-4-fluorophenoxy}-1,5-dimethylpyrazol-4-yl)-2-fluoro-3-methylphenyl]-3-[(tert-butoxycarbonyl)amino]propanoate (mixture, 600 mg, 0.66 mmol, 1.00 equiv.) in CH₂Cl₂ (10 mL) was added TFA (10.00 mL) dropwise at room temperature. The resulting mixture was stirred for 2 h at room temperature and then was concentrated to afford {5-[(4-{3-[(1S)-1-amino-3-ethoxy-3-oxopropyl]-4-fluoro-5-methylphenyl}-1,5-dimethylpyrazol-3-yl)oxy]-2-fluorophenyl}({5-[2-(3-fluoroazetidin-1-yl)ethyl]-2-oxo-4-(trifluoromethyl)pyridin-1-yl})acetic acid (mixture, 350 mg, crude) as a yellow solid. LC-MS: (ES+H, m/z) [M+H]⁺=750.1.

### Step 3: Preparation of rel-ethyl 2-[(13S,16S)-11,18-difluoro-13-{5-[2-(3-fluoroazetidin-1-yl)ethyl]-2-oxo-4-(trifluoromethyl)pyridin-1-yl}-3,4,19-trimethyl-14-oxo-7Z-oxa-4,5,15-triazatetracyclo[15.3.1.1\hat{ }{8,12}.0\hat{ }{2,6}]docosa-1(21),2,5,8,10,12(22),17,19-octaen-16-yl]acetate and rel-ethyl 2-[(13S,16S)-11,18-difluoro-13-{5-[2-(3-fluoroazetidin-1-yl)ethyl]-2-oxo-4-(trifluoromethyl)pyridin-1-yl}-3,4,19-trimethyl-14-oxo-7-oxa-4,5,15-triazatetracyclo[15.3.1.1\hat{ }{8,12}.0\hat{ }{2,6}]docosa-1(21),2,5,8,10,12(22),17,19-octaen-16-yl]acetate

To a stirred mixture of {5-[(4-{3-[(1S)-1-amino-3-ethoxy-3-oxopropyl]-4-fluoro-5-methylphenyl}-1,5-dimethylpyrazol-3-yl)oxy]-2-fluorophenyl}({5-[2-(3-fluoroazetidin-1-yl)ethyl]-2-oxo-4-(trifluoromethyl)pyridin-1-yl})acetic acid (mixture, 350 mg, 0.46 mmol, 1.00 equiv.) in MeCN (40 mL) were added EDCI (134 mg, 0.70 mmol, 1.50 equiv.), HOBT (82 mg, 0.60 mmol, 1.30 equiv.) and DIEA (181 mg, 1.40 mmol, 3.00 equiv.) in portions at room temperature. The resulting mixture was stirred for 2 h at room temperature and then diluted with EtOAc (100 mL). The mixture was washed with water (2×80 mL), and the organic layer was washed with brine (1×80 mL), dried over anhydrous Na₂SO₄, filtered, and concentrated. The residue was purified by reversed-phase flash chromatography (C18 gel; mobile phase, MeCN in water (0.1% NH₄HCO3), 0% to 60% gradient in 40 min; detector, UV 220 nm) to provide rel-ethyl 2-[(13S,16S)-11,18-difluoro-13-{5-[2-(3-fluoroazetidin-1-yl)ethyl]-2-oxo-4-(trifluoromethyl)pyridin-1-yl}-3,4,19-trimethyl-14-oxo-7-oxa-4,5,15-triazatetracyclo[15.3.1.1\hat{ }{8,12}.0\hat{ }{2,6}]docosa-1(21),2,5,8,10,12(22),17,19-octaen-16-yl]acetate (40 mg, crude) as a green solid and rel-ethyl 2-[(13S,16S)-11,18-difluoro-13-{5-[2-(3-fluoroazetidin-1-yl)ethyl]-2-oxo-4-(trifluoromethyl)pyridin-1-yl}-3,4,19-trimethyl-14-oxo-7-oxa-4,5,15-triazatetracyclo[15.3.1.1\hat{ }{8,12}.0\hat{ }{2,6}]docosa-1(21),2,5,8,10,12(22),17,19-octaen-16-yl]acetate (40 mg, crude) as a green solid.

rel-ethyl 2-[(13S,16S)-11,18-difluoro-13-{5-[2-(3-fluoroazetidin-1-yl)ethyl]-2-oxo-4-(trifluoromethyl)pyridin-1-yl}-3,4,19-trimethyl-14-oxo-7-oxa-4,5,15-triazatetracyclo[15.3.1.1\hat{ }{8,12}.0\hat{ }{2,6}]docosa-1(21),2,5,8,10,12(22),17,19-octaen-16-yl]acetate, (compound 4): LC-MS: (ES+H, m/z) [M+H]⁺=732.1. ¹H NMR (400 MHz, CD₃OD) δ7.66-7.62 (m, 1H), 7.50 (s, 1H), 7.29 (t, J=9.0 Hz, 1H), 7.06 (dd, J=7.0, 2.2 Hz, 1H), 6.94 (dd, J=6.0, 3.0 Hz, 1H), 6.89 (d, J=13.3 Hz, 2H), 6.49 (dd, J=6.8, 2.2 Hz, 1H), 5.71 (dd, J=9.9, 4.7 Hz, 1H), 4.97 (t, J=5.3 Hz, 1H), 4.21-4.09 (m, 2H), 3.77 (s, 3H), 3.48-3.34 (m, 2H), 3.07-2.97 (m, 2H), 2.77-2.65 (m, 2H), 2.56-2.44 (m, 4H), 2.32 (d, J=1.9 Hz, 3H), 2.15 (s, 3H), 1.23 (t, J=7.1 Hz, 3H).

rel-ethyl 2-[(13S,16S)-11,18-difluoro-13-{5-[2-(3-fluoroazetidin-1-yl)ethyl]-2-oxo-4-(trifluoromethyl)pyridin-1-yl}-3,4,19-trimethyl-14-oxo-7-oxa-4,5,15-triazatetracyclo[15.3.1.1\hat{ }{8,12}.0\hat{ }{2,6}]docosa-1(21),2,5,8,10,12(22),17,19-octaen-16-yl]acetate (compound 4B): LC-MS: (ES+H, m/z) [M+H]⁺=732.2. ¹H NMR (400 MHz, CD₃OD) δ8.21 (d, J=3.9 Hz, 1H), 7.81 (s, 1H), 7.28-7.20 (m, 4H), 7.12-7.05 (m, 3H), 5.17-5.02 (m, 1H), 4.52 (t, J=6.9 Hz, 1H), 4.03 (dd, J=7.1, 1.1 Hz, 2H), 3.75 (s, 3H), 3.67-3.61 (m, 2H), 3.27-3.19 (m, 2H), 2.81 (dd, J=22.2, 6.8 Hz, 4H), 2.66 (d, J=7.0 Hz, 2H), 2.34 (d, J=2.5 Hz, 3H), 2.22 (d, J=2.1 Hz, 3H), 1.13 (t, J=7.1 Hz, 3H).

### Step 4: Preparation of rel-[(13S,16S)-11,18-difluoro-13-{5-[2-(3-fluoroazetidin-1-yl)ethyl]-2-oxo-4-(trifluoromethyl)pyridin-1-yl}-3,4,19-trimethyl-14-oxo-7-oxa-4,5,15-triazatetracyclo[15.3.1.1\hat{ }{8,12}.0\hat{ }{2,6}]docosa-1(21),2,5,8,10,12(22),17,19-octaen-16-yl]acetic acid

To a stirred mixture of compound 4 (40 mg, 0.06 mmol, 1.00 equiv.) in THF (1 mL) and H₂O (0.3 mL) were added LiOH·H₂O (14 mg, 0.34 mmol, 5.00 equiv.) in portions at room temperature. The resulting mixture was stirred for 2 h at room temperature and then neutralized to pH 7 with 1 M HCl (aq.) and concentrated. The crude product (50 mg) was purified by Prep-HPLC(Column: XBridge Shield RP18 OBD Column 30*150 mm, 5 μm; Mobile Phase A: water (0.1% HCOOH), Mobile Phase B: MeCN; Flow rate: 60 mL/min; Gradient: 15% B to 35% B in 8 min; Wavelength: 254 nm/220 nm; RT1 (min) 9.38) to afford rel-[(13S,16S)-11,18-difluoro-13-{5-[2-(3-fluoroazetidin-1-yl)ethyl]-2-oxo-4-(trifluoromethyl)pyridin-1-yl}-3,4,19-trimethyl-14-oxo-7-oxa-4,5,15-triazatetracyclo[15.3.1.1\hat{ }{8,12}.0\hat{ }{2,6}]docosa-1(21),2,5,8,10,12(22),17,19-octaen-16-yl]acetic acid (19.9 mg, 40.7%) as a white solid. LC-MS: (ES+H, m/z) [M+H]⁺=704.15. ¹H NMR (300 MHz, CD₃OD) δ 7.67-7.62 (m, 1H), 7.51 (s, 1H), 7.29 (t, J=8.9 Hz, 1H), 7.06 (d, J=6.8 Hz, 1H), 7.01 (s, 1H), 6.99-6.94 (m, 1H), 6.91 (s, 1H), 6.52 (d, J=6.6 Hz, 1H), 5.70 (s, 1H), 5.20-4.80 (m, 1H), 3.79 (s, 3H), 3.69 (s, 2H), 3.56 (s, 2H), 2.76 (d, J=8.4 Hz, 2H), 2.72-2.61 (m, 2H), 2.56 (d, J=7.1 Hz, 2H), 2.33 (s, 3H), 2.16 (s, 3H). ¹⁹F NMR (282 MHz, CD₃OD) δ −65.00, −121.76, −127.12, −180.61.

### Example 41

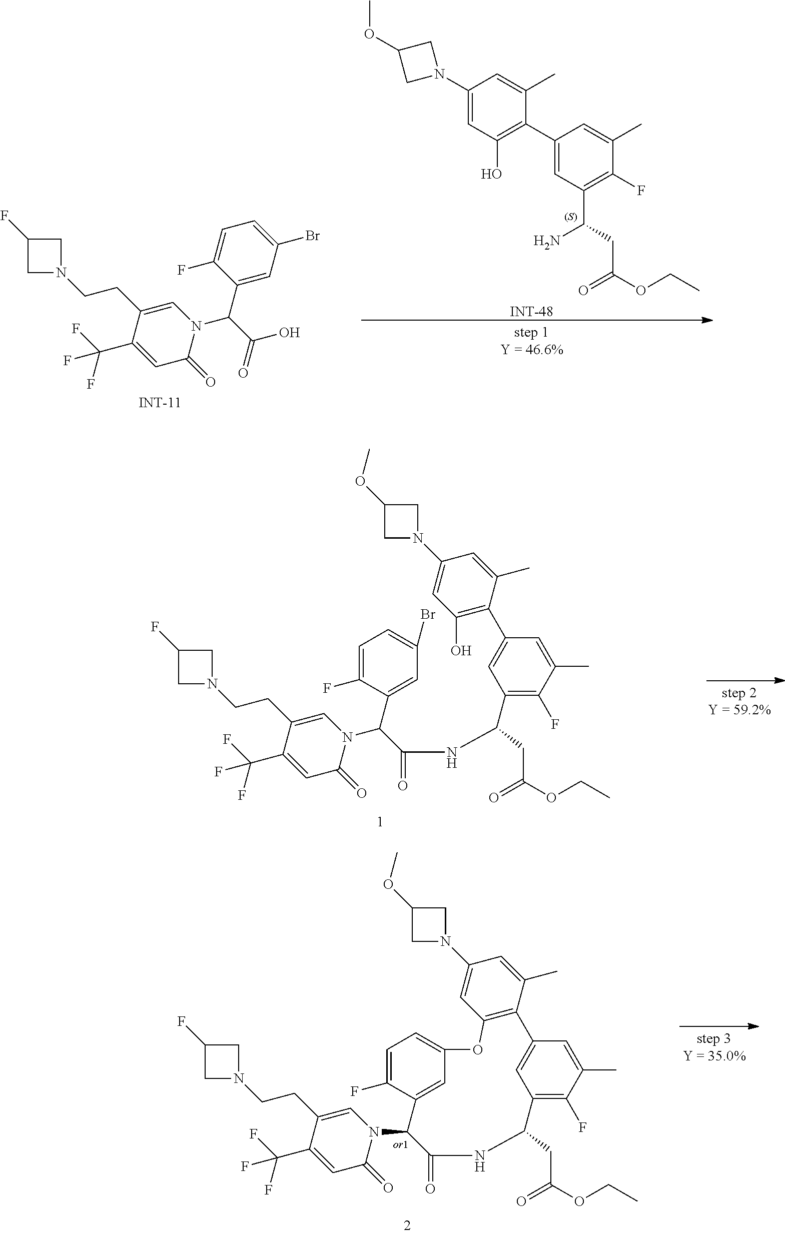

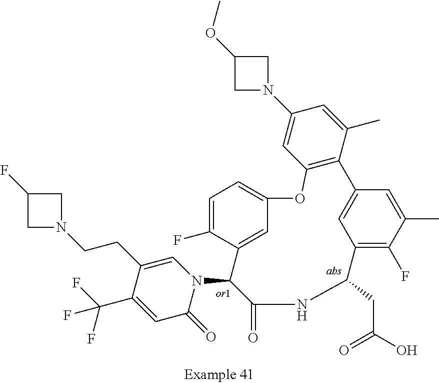 [SMILES: COC1CN(c2cc(C)c3c(c2)Oc2ccc(F)c(c2)[C@H](n2cc(CCN4CC(F)C4)c(C(F)(F)F)cc2=O)C(=O)N[C@@H](CC(=O)O)c2cc-3cc(C)c2F)C1]

### Step 1: Preparation of ethyl (3S)-3-[2-(5-bromo-2-fluorophenyl)-2-{5-[2-(3-fluoroazetidin-1-yl)ethyl]-2-oxo-4-(trifluoromethyl)pyridin-1-yl}acetamido]-3-[4-fluoro-2′-hydroxy-4′-(3-methoxyazetidin-1-yl)-5,6′-dimethyl-[1,1′-biphenyl]-3-yl]propanoate

A mixture of (5-bromo-2-fluorophenyl)({5-[2-(3-fluoroazetidin-1-yl)ethyl]-2-oxo-4-(trifluoromethyl)pyridin-1-yl})acetic acid (167 mg, 0.34 mmol, 1.00 equiv.), EDCI (129 mg, 0.67 mmol, 2.00 equiv.) and HOBT (91 mg, 0.67 mmol, 2.00 equiv.) in MeCN (5 mL) was stirred for 1 h at 30° C. under nitrogen atmosphere. To the above mixture was added ethyl (3S)-3-amino-3-[4-fluoro-2′-hydroxy-4′-(3-methoxyazetidin-1-yl)-5,6′-dimethyl-[1,1′-biphenyl]-3-yl]propanoate (140 mg, 0.34 mmol, 1.00 equiv.) in MeCN (3 mL) at 0° C. The resulting mixture was stirred overnight at 30° C. and then concentrated. The residue was purified by reversed-phase flash chromatography (C18 gel; mobile phase, MeCN in water (0.1% NH₃·H₂O), 40% to 80% gradient in 20 min; detector, UV 220 nm) to provide ethyl (3S)-3-[2-(5-bromo-2-fluorophenyl)-2-{5-[2-(3-fluoroazetidin-1-yl)ethyl]-2-oxo-4-(trifluoromethyl)pyridin-1-yl}acetamido]-3-[4-fluoro-2′-hydroxy-4′-(3-methoxyazetidin-1-yl)-5,6′-dimethyl-[1,1′-biphenyl]-3-yl]propanoate (140 mg, 46.6%, mixture) as a light brown solid. LC-MS: (ES+H, m/z) [M+H]⁺=893.15.

### Step 2: Preparation of ethyl 2-[(14S,17S)-12,19-difluoro-14-{5-[2-(3-fluoroazetidin-1-yl)ethyl]-2-oxo-4-(trifluoromethyl)pyridin-1-yl}-5-(3-methoxyazetidin-1-yl)-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate

A mixture of ethyl (3S)-3-[2-(5-bromo-2-fluorophenyl)-2-{5-[2-(3-fluoroazetidin-1-yl)ethyl]-2-oxo-4-(trifluoromethyl)pyridin-1-yl}acetamido]-3-[4-fluoro-2′-hydroxy-4′-(3-methoxyazetidin-1-yl)-5,6′-dimethyl-[1,1′-biphenyl]-3-yl]propanoate (130 mg, 0.15 mmol, 1.00 equiv.), CuI (14 mg, 0.07 mmol, 0.50 equiv.), K₂CO₃ (40 mg, 0.29 mmol, 2.00 equiv.) and (1R,2R)—N1,N2-dimethylcyclohexane-1,2-diamine (21 mg, 0.15 mmol, 1.00 equiv.) in 1,4-dioxane (5 mL) was stirred for 2 h at 100° C. under nitrogen atmosphere and then cooled to room temperature and concentrated. The residue was purified by reversed-phase flash chromatography (C18 gel; mobile phase, MeCN in water (0.1% NH₃·H₂O), 40% to 80% gradient in 20 min; detector, UV 220 nm) to provide ethyl 2-[(14S,17S)-12,19-difluoro-14-{5-[2-(3-fluoroazetidin-1-yl)ethyl]-2-oxo-4-(trifluoromethyl)pyridin-1-yl}-5-(3-methoxyazetidin-1-yl)-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (70 mg, 59.2%) as a light red solid. LC-MS: (ES+H, m/z) [M+H]⁺=813.25. ¹H NMR (300 MHz, CD₃OD) δ 7.58 (dt, J=9.1, 3.7 Hz, 1H), 7.37 (s, 1H), 7.17 (t, J=9.0 Hz, 1H), 6.94 (dd, J=7.2, 2.3 Hz, 1H), 6.83 (d, J=13.4 Hz, 2H), 6.54 (d, J=2.3 Hz, 1H), 6.41 (dd, J=6.5, 2.2 Hz, 1H), 6.27 (d, J=2.2 Hz, 1H), 6.08 (dd, J=6.1, 2.9 Hz, 1H), 5.68 (t, J=7.8 Hz, 1H), 4.99 (p, J=5.3 Hz, 1H), 4.36 (q, J=5.2, 4.7 Hz, 1H), 4.24-4.05 (m, 4H), 3.71 (td, J=8.8, 4.3 Hz, 2H), 3.36 (s, 5H), 3.18-2.90 (m, 3H), 2.88-2.64 (m, 3H), 2.59-2.46 (m, 3H), 2.43 (d, J=8.9 Hz, 2H), 2.31 (d, J=2.0 Hz, 3H), 1.91 (s, 3H), 1.18 (t, J=7.1 Hz, 3H). ¹⁹F NMR (282 MHz, CD₃OD) δ −64.98, −123.40, −127.45, −180.00.

### Step 3: Preparation of [(14S,17S)-12,19-difluoro-14-{5-[2-(3-fluoroazetidin-1-yl)ethyl]-2-oxo-4-(trifluoromethyl)pyridin-1-yl}-5-(3-methoxyazetidin-1-yl)-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid

A mixture of ethyl 2-[(14S,17S)-12,19-difluoro-14-{5-[2-(3-fluoroazetidin-1-yl)ethyl]-2-oxo-4-(trifluoromethyl)pyridin-1-yl}-5-(3-methoxyazetidin-1-yl)-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (50 mg, 0.06 mmol, 1.00 equiv.) and LiOH·H₂O (11 mg, 0.25 mmol, 4.00 equiv.) in THF (3 mL) and H₂O (1 mL) was stirred overnight at 30° C. under nitrogen atmosphere and then concentrated. The crude product (40 mg) was purified by Prep-HPLC (Column: XSelect CSH Prep C18 OBD Column, 30*150 mm, 5 μm; Mobile Phase A: water (0.1% HCOOH), Mobile Phase B: MeCN; Flow rate: 60 mL/min; Gradient: 20% B to 40% B in 10 min; Wavelength: 254 nm/220 nm; RT1 (min) 9.3) to afford [(14S,17S)-12,19-difluoro-14-{5-[2-(3-fluoroazetidin-1-yl)ethyl]-2-oxo-4-(trifluoromethyl)pyridin-1-yl}-5-(3-methoxyazetidin-1-yl)-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid (17.6 mg, 35.0%) as a white solid. LC-MS: (ES+H, m/z) [M+H]⁺=785.15. ¹H NMR (400 MHz, DMSO-d₆) δ 9.19 (d, J=7.3 Hz, 1H), 7.65 (dt, J=9.2, 3.6 Hz, 1H), 7.31-7.19 (m, 2H), 6.95 (dd, J=7.1, 2.1 Hz, 1H), 6.80 (s, 1H), 6.72-6.59 (m, 2H), 6.43-6.34 (m, 1H), 6.25 (d, J=2.2 Hz, 1H), 5.97 (dd, J=6.3, 3.0 Hz, 1H), 5.46 (q, J=7.4 Hz, 1H), 4.91-4.72 (m, 1H), 4.35 (p, J=5.4 Hz, 1H), 4.12 (ddd, J=14.5, 8.2, 6.1 Hz, 2H), 3.68 (ddd, J=21.1, 8.3, 4.3 Hz, 2H), 3.27 (s, 3H), 3.22-3.16 (m, 1H), 2.84 (dq, J=24.2, 6.0 Hz, 2H), 2.68-2.56 (m, 3H), 2.44-2.31 (m, 4H), 2.27 (s, 3H), 1.86 (s, 3H). ¹⁹F NMR (377 MHz, DMSO-d₆) δ −62.61, −121.91, −126.54, −176.89.

### Example 42

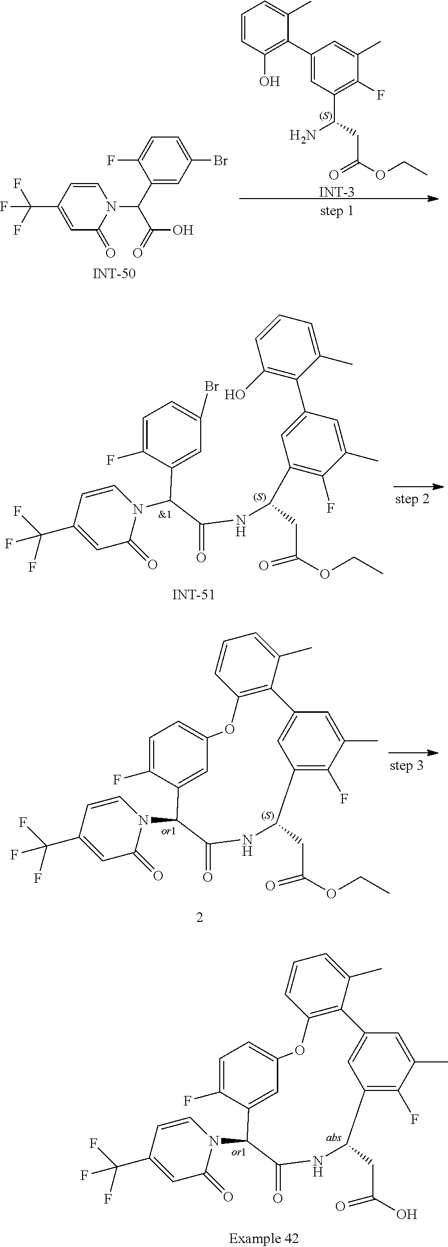

### Step 1: Preparation of ethyl (3S)-3-[2-(5-bromo-2-fluorophenyl)-2-[2-oxo-4-(trifluoromethyl)pyridin-1-yl]acetamido]-3-{4-fluoro-2′-hydroxy-5,6′-dimethyl-[1,1′-biphenyl]-3-yl}propanoate (Intermediate 51)

A mixture of (5-bromo-2-fluorophenyl)[2-oxo-4-(trifluoromethyl)pyridin-1-yl]acetic acid (600 mg, 1.522 mmol, 1.00 equiv.) and HOBT (411 mg, 3.044 mmol, 2.00 equiv.), EDCI (583 mg, 3.044 mmol, 2.00 equiv.) in MeCN (10 mL) was stirred for 10 min at 30° C. under nitrogen atmosphere. To the above mixture was added ethyl (3S)-3-amino-3-{4-fluoro-2′-hydroxy-5,6′-dimethyl-[1,1′-biphenyl]-3-yl}propanoate (504 mg, 1.522 mmol, 1.00 equiv.). The resulting mixture was stirred for an additional 2 h at 30° C. and then filtered. The filter cake was washed with MeCN (3×5 mL) and the combined filtrate was concentrated under reduced pressure. The residue was purified by reversed-phase flash chromatography (C18 gel; mobile phase, MeCN in water (0.1% NH₃·H₂O), 70%-90% gradient in 20 min; detector, UV 220 nm) to provide ethyl (3S)-3-[2-(5-bromo-2-fluorophenyl)-2-[2-oxo-4-(trifluoromethyl)pyridin-1-yl]acetamido]-3-{4-fluoro-2′-hydroxy-5,6′-dimethyl-[1,1′-biphenyl]-3-yl}propanoate as a brown oil (340 mg, 31.5%, mixture). LC-MS: (ES+H, m/z) [M+H]⁺=707.10.

### Step 2: Preparation of ethyl 2-[(14S,17S)-12,19-difluoro-3,20-dimethyl-15-oxo-14-[2-oxo-4-(trifluoromethyl)pyridin-1-yl]-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate

To a stirred solution of ethyl (3S)-3-[2-(5-bromo-2-fluorophenyl)-2-[2-oxo-4-(trifluoromethyl)pyridin-1-yl]acetamido]-3-{4-fluoro-2′-hydroxy-5,6′-dimethyl-[1,1′-biphenyl]-3-yl}propanoate (140 mg, 0.20 mmol, 1.00 equiv.) and (1R,2R)—N1,N2-dimethylcyclohexane-1,2-diamine (28 mg, 0.20 mmol, 1.00 equiv.) in 1,4-dioxane (5 mL) were added CuI (18 mg, 0.10 mmol, 0.50 equiv.) and K₂CO₃ (54 mg, 0.40 mmol, 2.00 equiv.) in portions at room temperature under nitrogen atmosphere. The resulting mixture was stirred overnight at 100° C. and then cooled to room temperature and concentrated. The residue was purified by reversed-phase flash chromatography (C18 gel; mobile phase, MeCN in water (0.1% NH₃·H₂O), 40% to 70% gradient in 20 min; detector, UV 220 nm) to provide ethyl 2-[(14S,17S)-12,19-difluoro-3,20-dimethyl-15-oxo-14-[2-oxo-4-(trifluoromethyl)pyridin-1-yl]-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (110 mg, 88.7%) as a yellow solid. LC-MS: (ES+H, m/z) [M+H]⁺=627.15. ¹H NMR (400 MHz, CD₃OD) δ 7.54 (dd, J=8.2, 4.4 Hz, 1H), 7.44 (d, J=8.0 Hz, 1H), 7.37-7.28 (m, 2H), 7.21-7.09 (m, 2H), 6.98 (d, J=6.8 Hz, 1H), 6.85 (s, 1H), 6.78 (s, 1H), 6.46-6.36 (m, 2H), 6.05 (dd, J=6.2, 3.0 Hz, 1H), 5.69 (t, J=7.9 Hz, 1H), 4.11-4.02 (m, 2H), 2.85 (dd, J=15.4, 8.3 Hz, 1H), 2.73 (dd, J=15.4, 7.4 Hz, 1H), 2.34 (d, J=2.0 Hz, 3H), 1.99 (s, 3H), 1.19 (t, J=7.1 Hz, 3H).

### Step 3: Preparation of [(14S,17S)-12,19-difluoro-3,20-dimethyl-15-oxo-14-[2-oxo-4-(trifluoromethyl)pyridin-1-yl]-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid

A mixture of ethyl 2-[(14S,17S)-12,19-difluoro-3,20-dimethyl-15-oxo-14-[2-oxo-4-(trifluoromethyl)pyridin-1-yl]-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (80 mg, 0.13 mmol, 1.00 equiv.) and LiOH·H₂O (16 mg, 0.38 mmol, 3.00 equiv.) in tetrahydrofuran (3 mL) and H₂O (1 mL) was stirred overnight at room temperature under nitrogen atmosphere. The mixture was acidified to pH 5 with 1 M HCl (aq.) and then concentrated. The crude product was purified by Prep-HPLC (Column: Xbridge Phenyl OBD Column, 19*150 mm, 5 μm; Mobile Phase A: water (10 mmol/L NH₄HCO₃+0.05% NH₃H₂0, Mobile Phase B: ACN; Flow rate: 60 mL/min; Gradient: 25% B to 35% B in 10 min; Wavelength: 254 nm/220 nm; RT1 (min) 10.43) to afford [(14S,17S)-12,19-difluoro-3,20-dimethyl-15-oxo-14-[2-oxo-4-(trifluoromethyl)pyridin-1-yl]-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid (30 mg, 39.5%) as a white solid. LC-MS: (ES+H, m/z) [M+H]⁺=599.15. ¹H NMR (300 MHz, DMSO-d₆) δ 9.47 (s, 1H), 7.58 (d, J=8.3 Hz, 2H), 7.42-7.29 (m, 2H), 7.29-7.14 (m, 2H), 6.98 (d, J=6.6 Hz, 1H), 6.85 (s, 1H), 6.76 (s, 1H), 6.45 (d, J=6.0 Hz, 1H), 6.33 (d, J=8.1 Hz, 1H), 5.96 (s, 1H), 5.45 (d, J=7.4 Hz, 1H), 2.48-2.45 (m, 2H), 2.29 (s, 3H), 1.94 (s, 3H). ¹⁹F NMR (282 MHz, DMSO-d₆) δ −65.433, −121.803, −126.079.

### Example 43

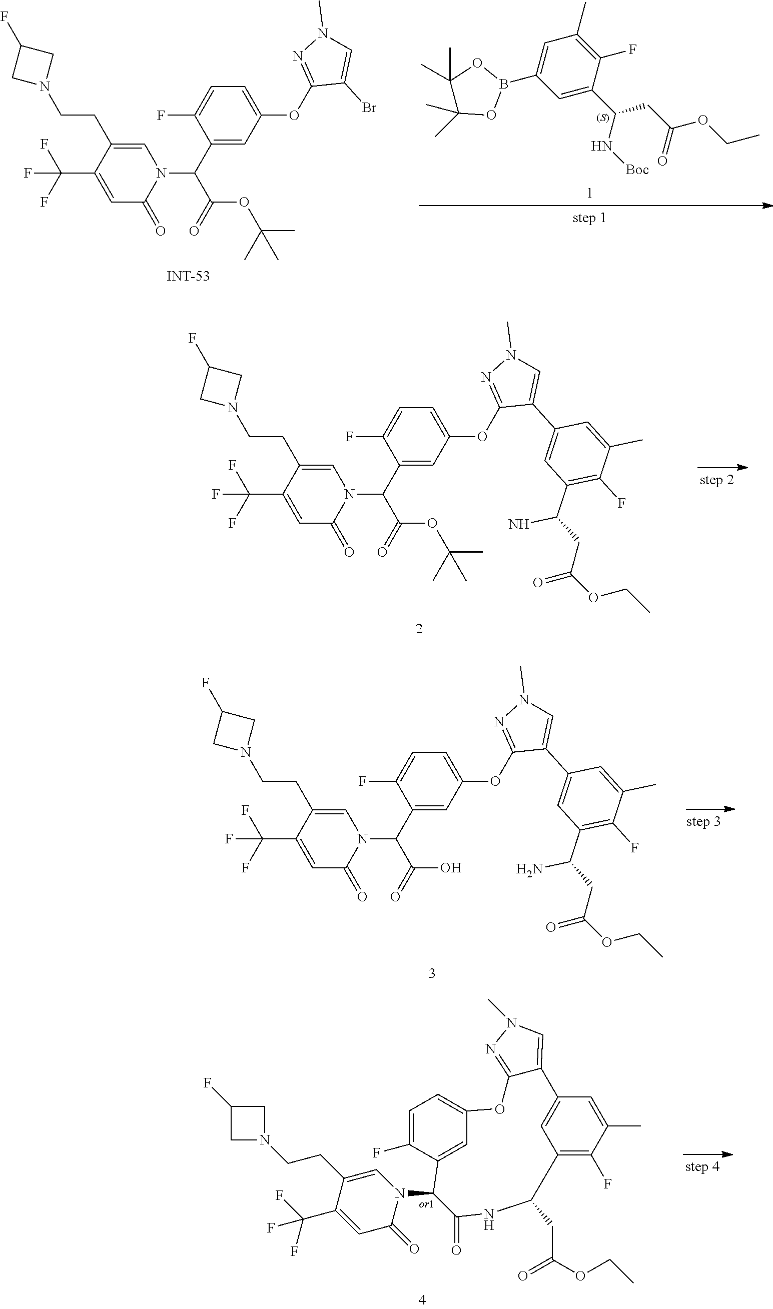

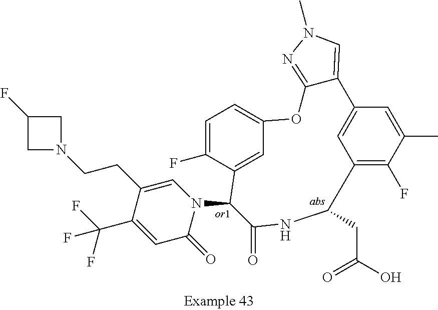 [SMILES: Cc1cc2cc(c1F)[C@H](CC(=O)O)NC(=O)[C@@H](n1cc(CCN3CC(F)C3)c(C(F)(F)F)cc1=O)c1cc(ccc1F)Oc1nn(C)cc1-2]

### Step 1: Preparation of ethyl (3S)-3-[5-(3-{3-[2-(tert-butoxy)-1-{5-[2-(3-fluoroazetidin-1-yl)ethyl]-2-oxo-4-(trifluoromethyl)pyridin-1-yl}-2-oxoethyl]-4-fluorophenoxy}-1-methylpyrazol-4-yl)-2-fluoro-3-methylphenyl]-3-[(tert-butoxycarbonyl)amino]propanoate

To a stirred solution of tert-butyl 2-{5-[(4-bromo-1-methylpyrazol-3-yl)oxy]-2-fluorophenyl}-2-{5-[2-(3-fluoroazetidin-1-yl)ethyl]-2-oxo-4-(trifluoromethyl)pyridin-1-yl}acetate (200 mg, 0.31 mmol, 1.00 equiv.) and ethyl (3S)-3-[(tert-butoxycarbonyl)amino]-3-[2-fluoro-3-methyl-5-(4,4,5,5-tetramethyl-1,3,2-dioxaborolan-2-yl)phenyl]propanoate (139 mg, 0.31 mmol, 1.00 equiv.) in 1,4-dioxane (5 mL) and H₂O (0.5 mL) were added K₂CO₃ (128 mg, 0.93 mmol, 3.00 equiv.) and Pd(dppf)Cl₂·CH₂Cl₂ (25 mg, 0.03 mmol, 0.10 equiv.) at room temperature under nitrogen atmosphere. The resulting mixture was stirred for 2 h at 100° C. under nitrogen atmosphere and then cooled to room temperature. The resulting mixture was concentrated under reduced pressure and purified by reversed-phase flash chromatography (C18 gel; mobile phase, CH₃CN in water (0.1% NH₃·H₂O), 45% to 100% gradient in 30 min; detector, UV 254 nm) to provide ethyl (3S)-3-[5-(3-{3-[2-(tert-butoxy)-1-{5-[2-(3-fluoroazetidin-1-yl)ethyl]-2-oxo-4-(trifluoromethyl)pyridin-1-yl}-2-oxoethyl]-4-fluorophenoxy}-1-methylpyrazol-4-yl)-2-fluoro-3-methylphenyl]-3-[(tert-butoxycarbonyl)amino]propanoate (200 mg, 72.6%) as a yellow solid. LC-MS: (ES+H, m/z) [M+H]⁺=892.4.

### Step 2: Preparation of {5-[(4-{3-[(1S)-1-amino-3-ethoxy-3-oxopropyl]-4-fluoro-5-methylphenyl}-1-methylpyrazol-3-yl) oxy]-2-fluorophenyl}({5-[2-(3-fluoroazetidin-1-yl)ethyl]-2-oxo-4-(trifluoromethyl)pyridin-1-yl})acetic acid

To a stirred solution of ethyl (3S)-3-[5-(3-{3-[2-(tert-butoxy)-1-{5-[2-(3-fluoroazetidin-1-yl) ethyl]-2-oxo-4-(trifluoromethyl) pyridin-1-yl}-2-oxoethyl]-4-fluorophenoxy}-1-methylpyrazol-4-yl)-2-fluoro-3-methylphenyl]-3-[(tert-butoxycarbonyl) amino]propanoate (240 mg, 0.27 mmol, 1.00 equiv.) in CH₂Cl₂ (6 mL) was added trifluoroacetic acid (2 mL) dropwise at 0° C. under nitrogen atmosphere. The resulting mixture was stirred overnight at room temperature under nitrogen atmosphere and then concentrated. The residue was purified by reversed-phase flash chromatography (C18 gel; mobile phase, MeCN in water (0.1% HCOOH), 10% to 50% gradient in 10 min; detector, UV 220 nm) to afford {5-[(4-{3-[(1S)-1-amino-3-ethoxy-3-oxopropyl]-4-fluoro-5-methylphenyl}-1-methylpyrazol-3-yl) oxy]-2-fluorophenyl} ({5-[2-(3-fluoroazetidin-1-yl)ethyl]-2-oxo-4-(trifluoromethyl)pyridin-1-yl})acetic acid (160 mg, 84.3%) as a yellow solid. LC-MS: (ES+H, m/z) [M+H]⁺=736.20.

### Step 3: Preparation of ethyl 2-[(16S)-11,18-difluoro-13-{5-[2-(3-fluoroazetidin-1-yl) ethyl]-2-oxo-4-(trifluoromethyl)pyridin-1-yl}-4,19-dimethyl-14-oxo-7-oxa-4,5,15-triazatetracyclo[15.3.1.1\hat{ }{8,12}.0\hat{ }{2,6}]docosa-1(21),2,5,8,10,12(22),17,19-octaen-16-yl]acetate

To a stirred solution of {5-[(4-{3-[(1S)-1-amino-3-ethoxy-3-oxopropyl]-4-fluoro-5-methylphenyl}-1-methylpyrazol-3-yl)oxy]-2-fluorophenyl}({5-[2-(3-fluoroazetidin-1-yl)ethyl]-2-oxo-4-(trifluoromethyl)pyridin-1-yl})acetic acid (120 mg, 0.163 mmol, 1.00 equiv.) in MeCN (5 mL) was added N,N-diisopropylethylamine (63 mg, 0.48 mmol, 3.00 equiv.), EDCI (47 mg, 0.24 mmol, 1.50 equiv.), and HOBT (29 mg, 0.21 mmol, 1.30 equiv.) at room temperature. The resulting mixture was stirred for 6 h at 60° C. under nitrogen atmosphere. The mixture was then cooled to room temperature, diluted with water (20 mL) and extracted with EtOAc (3×20 mL). The combined organic layers were concentrated and the crude product was purified by reversed-phase flash chromatography (C18 gel; mobile phase, MeCN in water (0.1% NH₃·H₂O), 10% to 50% gradient in 10 min; detector, UV 220 nm) to afford ethyl 2-[(16S)-11,18-difluoro-13-{5-[2-(3-fluoroazetidin-1-yl) ethyl]-2-oxo-4-(trifluoromethyl)pyridin-1-yl}-4,19-dimethyl-14-oxo-7-oxa-4,5,15-triazatetracyclo[15.3.1.1\hat{ }{8,12}.0\hat{ }{2,6}]docosa-1(21),2,5,8,10,12(22),17,19-octaen-16-yl]acetate (35 mg, 29.9%) as a yellow solid. LC-MS: (ES+H, m/z) [M+H]⁺=718.35.

### Step 4: Preparation of [(13S,16S)-11,18-difluoro-13-{5-[2-(3-fluoroazetidin-1-yl)ethyl]-2-oxo-4-(trifluoromethyl)pyridin-1-yl}-4,19-dimethyl-14-oxo-7-oxa-4,5,15-triazatetracyclo[15.3.1.1\hat{ }{8,12}.0\hat{ }{2,6}]docosa-1(21),2,5,8,10,12(22),17,19-octaen-16-yl]acetic acid

To a stirred solution of ethyl 2-[(16S)-11,18-difluoro-13-{5-[2-(3-fluoroazetidin-1-yl)ethyl]-2-oxo-4-(trifluoromethyl)pyridin-1-yl}-4,19-dimethyl-14-oxo-7-oxa-4,5,15-triazatetracyclo[15.3.1.1\hat{ }{8,12}.0\hat{ }{2,6}]docosa-1(21),2,5,8,10,12(22),17,19-octaen-16-yl]acetate (30 mg, 0.04 mmol, 1.00 equiv.) in THF (2 mL) and H₂O (0.4 mL) was added LiOH·H₂O (7 mg, 0.16 mmol, 4.00 equiv.) at room temperature under nitrogen atmosphere. The resulting mixture was stirred for 2 h at room temperature and then acidified to pH 5 with 1 M HCl (aq.) and concentrated. The residue was purified by reversed-phase flash chromatography ((Column: XBridge Shield RP18 OBD Column 30*150 mm, 5 μm; Mobile Phase A: water (0.1% NH₃·H₂O+10 mmol/L NH₄HCO₃), Mobile Phase B: MeCN; Flow rate: 60 mL/min; Gradient: 24% B to 46% B in 8 min; Wavelength: 254 nm/220 nm) to afford [(13S,16S)-11,18-difluoro-13-{5-[2-(3-fluoroazetidin-1-yl)ethyl]-2-oxo-4-(trifluoromethyl)pyridin-1-yl}-4,19-dimethyl-14-oxo-7-oxa-4,5,15-triazatetracyclo[15.3.1.1\hat{ }{8,12}.0\hat{ }{2,6}]docosa-1(21),2,5,8,10,12(22),17,19-octaen-16-yl]acetic acid (4 mg, 13.7%) as a white solid. LC-MS: (ES+H, m/z) [M+H]⁺=690.20. ¹H NMR (400 MHz, DMSO-d₆) δ 9.54-9.26 (m, 1H), 7.68 (s, 1H), 7.56-7.49 (m, 1H), 7.47 (s, 1H), 7.40-7.29 (m, 1H), 7.09 (d, J=6.8 Hz, 1H), 6.98-6.75 (m, 3H), 6.48 (d, J=6.7 Hz, 1H), 5.47 (d, J=9.7 Hz, 1H), 4.80-5.00 (m, 1H), 3.81 (s, 3H), 3.18 (s, 2H), 3.10-2.78 (m, 3H), 2.50-2.30 (m, 6H), 2.26 (s, 3H). ¹⁹F NMR (377 MHz, DMSO-d₆) −62.63, −120.52, −126.16, −177.35.

### Example 44

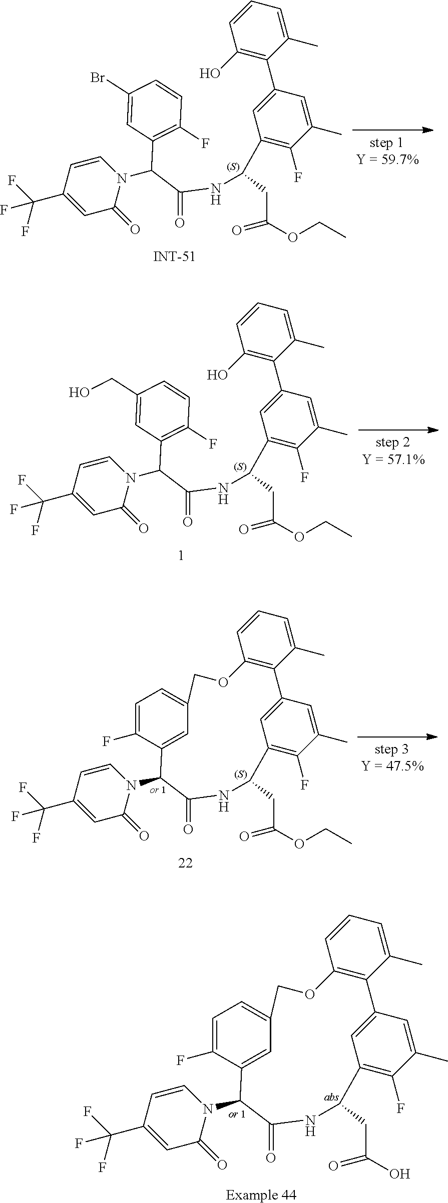

### Step 1: Preparation of ethyl (3S)-3-{4-fluoro-2′-hydroxy-5,6′-dimethyl-[1,1′-biphenyl]-3-yl}-3-{2-[2-fluoro-5-(hydroxymethyl)phenyl]-2-[2-oxo-4-(trifluoromethyl)pyridin-1-yl]acetamido}propanoate

A mixture of ethyl (3S)-3-[2-(5-bromo-2-fluorophenyl)-2-[2-oxo-4-(trifluoromethyl)pyridin-1-yl]acetamido]-3-{4-fluoro-2′-hydroxy-5,6′-dimethyl-[1,1′-biphenyl]-3-yl}propanoate (180 mg, 0.25 mmol, 1.00 equiv.), (tributylstannyl)methanol (245 mg, 0.76 mmol, 3.00 equiv.) and dicyclohexyl[2′,4′,6′-tris(propan-2-yl)-[1,1′-biphenyl]-2-yl]phosphane; {2′-amino-[1,1′-biphenyl]-2-yl}(chloro)palladium (40 mg, 0.05 mmol, 0.20 equiv.) in 1,4-dioxane (8 mL) was stirred for 12 h at 80° C. under nitrogen atmosphere and then cooled to room temperature. The resulting mixture was diluted with water (5 mL) and extracted with EtOAc (3×15 mL). The combined organic layers were washed with brine (2×10 mL), dried over anhydrous Na₂SO₄, filtered, and concentrated. The residue was purified by reversed-phase flash chromatography (C18 gel; mobile phase, MeCN in water (0.10% NH₃·H₂O), 35% to 75% gradient in 20 min; detector, UV 220 nm) to provide ethyl (3S)-3-{4-fluoro-2′-hydroxy-5,6′-dimethyl-[1,1′-biphenyl]-3-yl}-3-{2-[2-fluoro-5-(hydroxymethyl)phenyl]-2-[2-oxo-4-(trifluoromethyl)pyridin-1-yl]acetamido}propanoate (100 mg, 59.7%, mixture) as a white solid. LC-MS: (ES+H, m/z) [M+H]⁺=659.40.

### Step 2: Preparation of ethyl 2-[(15S,18S)-13,20-difluoro-3,21-dimethyl-16-oxo-15-[2-oxo-4-(trifluoromethyl)pyridin-1-yl]-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetate

A mixture of ethyl (3S)-3-{4-fluoro-2′-hydroxy-5,6′-dimethyl-[1,1′-biphenyl]-3-yl}-3-{2-[2-fluoro-5-(hydroxymethyl)phenyl]-2-[2-oxo-4-(trifluoromethyl)pyridin-1-yl]acetamido}propanoate (90 mg, 0.14 mmol, 1.00 equiv., mixture) and 2-(tributyl-lambda5-phosphanylidene)acetonitrile (165 mg, 0.69 mmol, 5.00 equiv.) in toluene (5 mL) was stirred overnight at 100° C. under nitrogen atmosphere and then cooled to room temperature and concentrated. The residue was purified by reversed-phase flash chromatography (C18 gel; mobile phase, MeCN in water (0.1% NH₃·H₂O), 30% to 70% gradient in 20 min; detector, UV 220 nm) to provide ethyl 2-[(15S,18S)-13,20-difluoro-3,21-dimethyl-16-oxo-15-[2-oxo-4-(trifluoromethyl)pyridin-1-yl]-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetate (50 mg, 57.1%) as light yellow solid. LC-MS: (ES+H, m/z) [M+H]⁺=641.30. ¹H NMR (400 MHz, CD₃OD) δ 7.51-7.44 (m, 1H), 7.31-7.24 (m, 2H), 7.20-7.08 (m, 3H), 7.02 (s, 1H), 6.96 (dd, J=7.0, 2.2 Hz, 1H), 6.84-6.79 (m, 2H), 6.59 (dd, J=6.9, 2.1 Hz, 1H), 6.37 (dd, J=7.4, 2.1 Hz, 1H), 5.77 (dd, J=9.5, 5.4 Hz, 1H), 5.30 (d, J=13.2 Hz, 1H), 4.96 (d, J=13.2 Hz, 1H), 4.20-4.04 (m, 2H), 2.87-2.63 (m, 2H), 2.03 (s, 3H), 1.96 (s, 3H), 1.21 (t, J=7.1 Hz, 3H). ¹⁹F NMR (377 MHz, CD₃OD) δ −68.26, −118.49, −128.46.

### Step 3: Preparation of [(15S,18S)-13,20-difluoro-3,21-dimethyl-16-oxo-15-[2-oxo-4-(trifluoromethyl)pyridin-1-yl]-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetic acid

A mixture of ethyl 2-[(15S,18S)-13,20-difluoro-3,21-dimethyl-16-oxo-15-[2-oxo-4-(trifluoromethyl)pyridin-1-yl]-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetate (50 mg, 0.08 mmol, 1.00 equiv.) and LiOH·H₂O (10 mg, 0.23 mmol, 3.00 equiv.) in THF (4 mL) and H₂O (1 mL) was stirred for 6 h at room temperature under nitrogen atmosphere. The mixture was acidified to pH 5 with 1 M HCl (aq.) and then concentrated. The crude product (50 mg) was purified by Prep-HPLC (Column: Xbridge Phenyl OBD Column, 19*150 mm, 5 μm; Mobile Phase A: water (10 mmol/L NH₄HCO₃+0.05% NH₃H₂0, Mobile Phase B: MeCN; Flow rate: 60 mL/min; Gradient: 25% B to 40% B in 10 min; Wavelength: 254 nm/220 nm; RT1 (min) 10.63) to afford [(15S,18S)-13,20-difluoro-3,21-dimethyl-16-oxo-15-[2-oxo-4-(trifluoromethyl)pyridin-1-yl]-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetic acid (23.1 mg, 47.5%) as a white solid. LC-MS: (ES+H, m/z) [M+H]⁺=613.25. ¹H NMR (400 MHz, DMSO-d₆) δ 9.52 (s, 1H), 7.56-7.48 (m, 1H), 7.29 (dd, J=15.0, 7.8 Hz, 2H), 7.23-7.09 (m, 3H), 7.05-6.95 (m, 2H), 6.88-6.79 (m, 2H), 6.48 (dd, J=6.8, 2.2 Hz, 1H), 6.34 (dd, J=7.4, 2.1 Hz, 1H), 5.59-5.51 (m, 1H), 5.36 (d, J=13.2 Hz, 1H), 4.97 (d, J=13.2 Hz, 1H), 2.62-2.52 (m, 2H), 2.28 (s, 3H), 1.90 (s, 3H). ¹⁹F NMR (377 MHz, DMSO-d₆) δ −65.42, −117.26, −127.33.

### Example 45

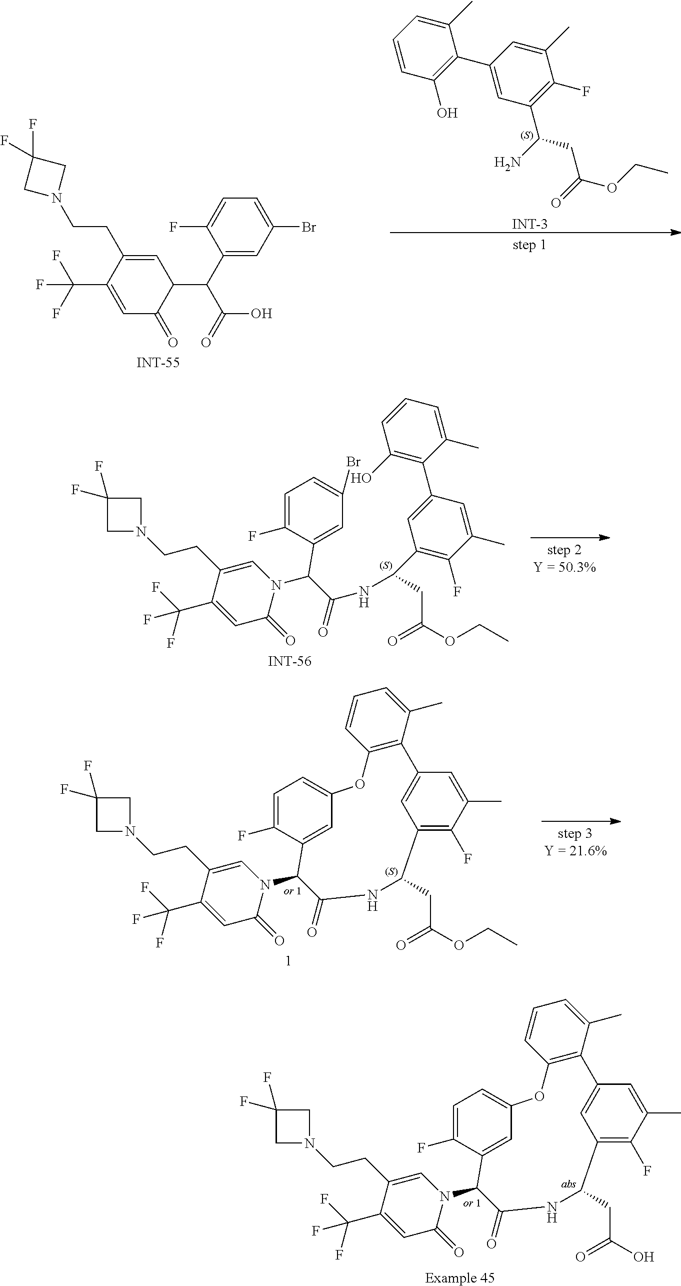

### Step 1: Preparation of ethyl (3S)-3-[2-(5-bromo-2-fluorophenyl)-2-{5-[2-(3,3-difluoroazetidin-1-yl)ethyl]-2-oxo-4-(trifluoromethyl)pyridin-1-yl}acetamido]-3-{4-fluoro-2′-hydroxy-5,6′-dimethyl-[1,1′-biphenyl]-3-yl}propanoate

To a stirred mixture of (5-bromo-2-fluorophenyl)({5-[2-(3,3-difluoroazetidin-1-yl)ethyl]-2-oxo-4-(trifluoromethyl)pyridin-1-yl})acetic acid (600 mg, 1.16 mmol, 1.00 equiv.) in MeCN (3 mL) was added EDCI (448 mg, 2.33 mmol, 2.00 equiv.) and HOBT (315 mg, 2.33 mmol, 2.00 equiv.) at room temperature under nitrogen atmosphere. The resulting mixture was stirred for 1 h at room temperature under nitrogen atmosphere and then treated with ethyl (3S)-3-amino-3-{4-fluoro-2′-hydroxy-5,6′-dimethyl-[1,1′-biphenyl]-3-yl}propanoate (503 mg, 1.52 mmol, 1.30 equiv.) and Et₃N (709 mg, 7.01 mmol, 6.00 equiv.) in MeCN (3 mL) dropwise over 1 min at room temperature. The resulting mixture was stirred for an additional 2 h at room temperature and then was concentrated. The residue was purified by reversed-phase flash chromatography (C18 gel; mobile phase, MeCN in water (0.1% NH₃·H₂O), 50% to 70% gradient in 20 min; detector, UV 254/220 nm) to afford ethyl (3S)-3-[2-(5-bromo-2-fluorophenyl)-2-{5-[2-(3,3-difluoroazetidin-1-yl)ethyl]-2-oxo-4-(trifluoromethyl)pyridin-1-yl}acetamido]-3-{4-fluoro-2′-hydroxy-5,6′-dimethyl-[1,1′-biphenyl]-3-yl}propanoate (370 mg, 38.2%, mixture) as colorless oil. LC-MS: (ES+H, m/z) [M+H]⁺=826.0.

### Step 2: Preparation of ethyl 2-[(14S,17S)-14-{5-[2-(3,3-difluoroazetidin-1-yl)ethyl]-2-oxo-4-(trifluoromethyl)pyridin-1-yl}-12,19-difluoro-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate

To a stirred mixture of ethyl (3S)-3-[2-(5-bromo-2-fluorophenyl)-2-{5-[2-(3,3-difluoroazetidin-1-yl)ethyl]-2-oxo-4-(trifluoromethyl)pyridin-1-yl}acetamido]-3-{4-fluoro-2′-hydroxy-5,6′-dimethyl-[1,1′-biphenyl]-3-yl}propanoate (110 mg, 0.13 mmol, 1.00 equiv.) in 1,4-dioxane (5 mL) were added CuI (13 mg, 0.06 mmol, 0.50 equiv.), K₂CO₃ (37 mg, 0.26 mmol, 2.00 equiv.) and (1R,2R)—N1,N2-dimethylcyclohexane-1,2-diamine (19 mg, 0.13 mmol, 1.00 equiv.) at room temperature under nitrogen atmosphere. The resulting mixture was stirred overnight at 100° C. under nitrogen atmosphere and then cooled to room temperature and concentrated. The residue was purified by reversed-phase flash chromatography (C18 gel; mobile phase, MeCN in water (0.1% NH₃·H₂O), 10% to 50% gradient in 10 min; detector, UV 254/220 nm) to afford ethyl 2-[(14S,17S)-14-{5-[2-(3,3-difluoroazetidin-1-yl)ethyl]-2-oxo-4-(trifluoromethyl)pyridin-1-yl}-12,19-difluoro-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (50 mg, 50.3%) as a colorless oil. LC-MS: (ES+H, m/z) [M+H]⁺=746.3.

### Step 3: Preparation of [(14S,17S)-14-{5-[2-(3,3-difluoroazetidin-1-yl)ethyl]-2-oxo-4-(trifluoromethyl)pyridin-1-yl}-12,19-difluoro-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid

To a stirred mixture of ethyl 2-[(14S,17S)-14-{5-[2-(3,3-difluoroazetidin-1-yl)ethyl]-2-oxo-4-(trifluoromethyl)pyridin-1-yl}-12,19-difluoro-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (40 mg, 0.05 mmol, 1.00 equiv.) in THF (1 mL) was added LiOH·H₂O (3 mg, 0.10 mmol, 2.00 equiv.) in water (0.5 mL) at room temperature under nitrogen atmosphere. The resulting mixture was stirred for 1 h at room temperature and then diluted with water (2 mL), acidified to pH 5 with 1 M HCl (aq.) and concentrated. The crude product was purified by Prep-HPLC (Column: XBridge Prep OBD C18 Column, 30*150 mm, 5 μm; Mobile Phase A: water (10 mmol/L NH₄HCO₃+0.05% NH₃H2O, Mobile Phase B: MeCN; Flow rate: 60 mL/min; Gradient: 33% B to 48% B in 10 min; Wavelength: 254 nm/220 nm; RT1 (min) 9.37) to afford [(14S,17S)-14-{5-[2-(3,3-difluoroazetidin-1-yl)ethyl]-2-oxo-4-(trifluoromethyl)pyridin-1-yl}-12,19-difluoro-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid (8.4 mg, 21.6%) as an off-white solid. LC-MS: (ES+H, m/z) [M+H]⁺=718.20. ¹H NMR (400 MHz, CD₃OD) δ 7.48-7.41 (m, 1H), 7.34 (d, J=7.5 Hz, 1H), 7.27-7.18 (m, 2H), 7.10-7.01 (m, 2H), 6.86 (dd, J=6.8, 2.1 Hz, 1H), 6.74 (d, J=18.6 Hz, 2H), 6.38 (dd, J=6.3, 2.2 Hz, 1H), 6.02 (dd, J=6.2, 2.9 Hz, 1H), 5.62 (t, J=7.4 Hz, 1H), 3.30 (td, J=12.2, 1.5 Hz, 4H), 2.67-2.48 (m, 4H), 2.38 (t, J=6.8 Hz, 2H), 2.24 (d, J=1.9 Hz, 3H), 1.90 (s, 3H). ¹⁹F NMR (377 MHz, CD₃OD) δ −65.01, −100.28, −123.38, −126.87.

### Example 46

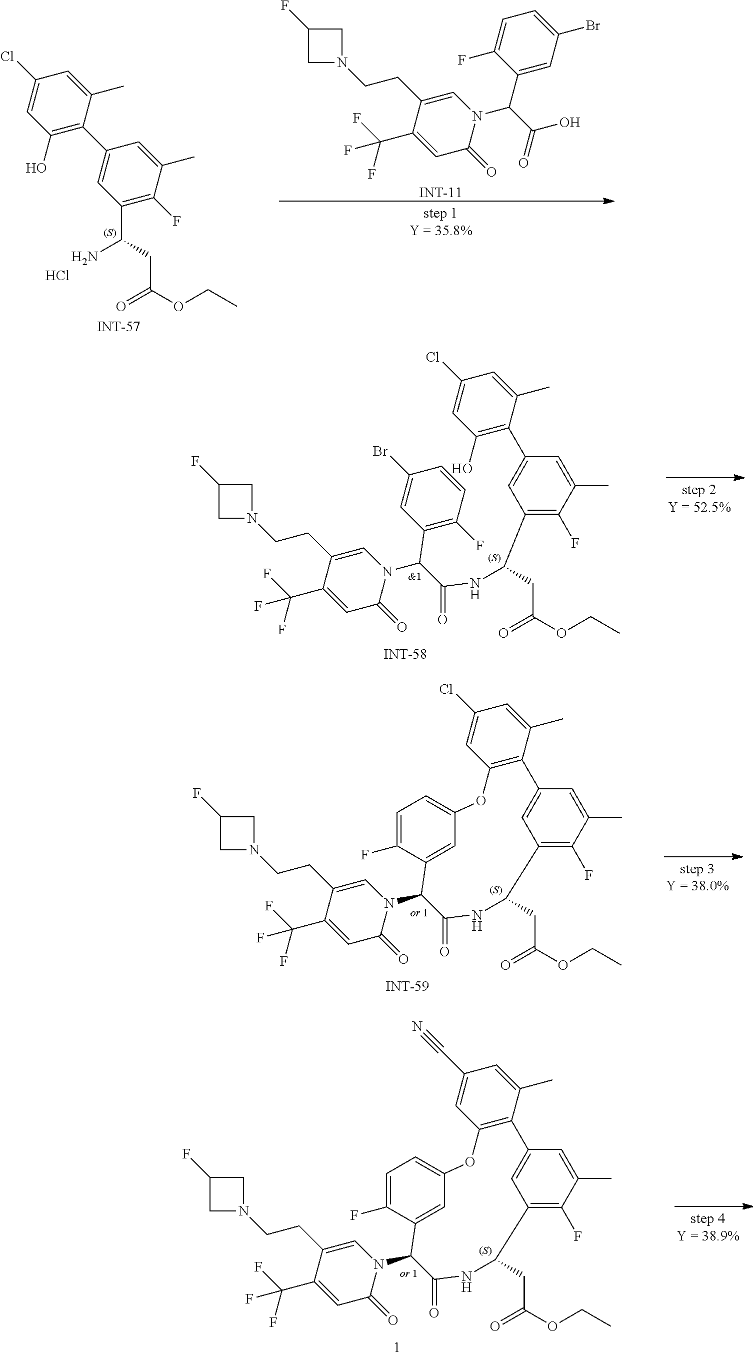

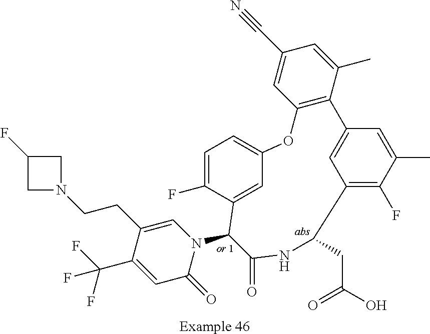

### Step 1: Preparation of ethyl (3S)-3-[2-(5-bromo-2-fluorophenyl)-2-{5-[2-(3-fluoroazetidin-1-yl)ethyl]-2-oxo-4-(trifluoromethyl)pyridin-1-yl}acetamido]-3-{4′-chloro-4-fluoro-2′-hydroxy-5,6′-dimethyl-[1,1′-biphenyl]-3-yl}propanoate

A mixture of ethyl (3S)-3-amino-3-{4′-chloro-4-fluoro-2′-hydroxy-5,6′-dimethyl-[1,1′-biphenyl]-3-yl}propanoate hydrochloride (800 mg, 1.99 mmol, 1.00 equiv.) and NEt₃ (1.70 mL, 11.93 mmol, 6.00 equiv.) in MeCN (3 mL) was stirred for 30 min at 30° C. under nitrogen atmosphere. The resulting mixture was concentrated under reduced pressure to provide ethyl (3S)-3-amino-3-{4′-chloro-4-fluoro-2′-hydroxy-5,6′-dimethyl-[1,1′-biphenyl]-3-yl}propanoate. In a separate vial, a mixture of (5-bromo-2-fluorophenyl)({5-[2-(3-fluoroazetidin-1-yl)ethyl]-2-oxo-4-(trifluoromethyl)pyridin-1-yl})acetic acid (985 mg, 1.99 mmol, 1.00 equiv.), HOBT (262 mg, 1.94 mmol, 1.30 equiv.) and EDCI (429 mg, 2.24 mmol, 1.50 equiv.) in MeCN (3 mL) was stirred for 1 h at 30° C. under nitrogen atmosphere. To the above mixture was added ethyl (3S)-3-amino-3-{4′-chloro-4-fluoro-2′-hydroxy-5,6′-dimethyl-[1,1′-biphenyl]-3-yl}propanoate in MeCN. The resulting mixture was stirred overnight at 40° C. and then concentrated. The residue was purified by reversed-phase flash chromatography (C18 gel; mobile phase, MeCN in water (0.1% NH₃·H₂O), 50% to 70% gradient in 30 min; detector, UV 220 nm) to provide ethyl (3S)-3-[2-(5-bromo-2-fluorophenyl)-2-{5-[2-(3-fluoroazetidin-1-yl)ethyl]-2-oxo-4-(trifluoromethyl)pyridin-1-yl}acetamido]-3-{4′-chloro-4-fluoro-2′-hydroxy-5,6′-dimethyl-[1,1′-biphenyl]-3-yl}propanoate (600 mg, 35.8%) as a white solid. LC-MS: (ES+H, m/z) [M+H]⁺=842.1.

### Step 2: Preparation of ethyl 2-[(14R*,17S)-5-chloro-12,19-difluoro-14-{5-[2-(3-fluoroazetidin-1-yl)ethyl]-2-oxo-4-(trifluoromethyl)pyridin-1-yl}-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate

To a stirred mixture of ethyl (3S)-3-[2-(5-bromo-2-fluorophenyl)-2-{5-[2-(3-fluoroazetidin-1-yl)ethyl]-2-oxo-4-(trifluoromethyl)pyridin-1-yl}acetamido]-3-{4′-chloro-4-fluoro-2′-hydroxy-5,6′-dimethyl-[1,1′-biphenyl]-3-yl}propanoate (400 mg, 0.47 mmol, 1.00 equiv.) and (1R,2R)—N1,N2-dimethylcyclohexane-1,2-diamine (67 mg, 0.47 mmol, 1.00 equiv.) in dioxane (10 mL) were added CuI (45 mg, 0.24 mmol, 0.50 equiv.) and K₂CO₃ (197 mg, 1.42 mmol, 3.00 equiv.) at room temperature under nitrogen atmosphere. The resulting mixture was stirred overnight at 100° C. and then cooled to room temperature and filtered. The filter cake was washed with MeCN (3×30 mL) and the combined filtrate was concentrated under reduced pressure. The residue was purified by reversed-phase flash chromatography (C18 gel; mobile phase, MeCN in water (0.11% NH₃·H₂O), 40% to 60% gradient in 10 min; detector, UV 220 nm) to provide ethyl 2-[(14R*,17S)-5-chloro-12,19-difluoro-14-{5-[2-(3-fluoroazetidin-1-yl)ethyl]-2-oxo-4-(trifluoromethyl)pyridin-1-yl}-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (190 mg, 52.5%) as a white solid. LC-MS: (ES+H, m/z) [M+H]⁺=762.2.

### Step 3: Preparation of ethyl 2-[(14S,17S)-5-cyano-12,19-difluoro-14-{5-[2-(3-fluoroazetidin-1-yl)ethyl]-2-oxo-4-(trifluoromethyl)pyridin-1-yl}-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate

To a stirred mixture of ethyl 2-[(14S,17S)-5-chloro-12,19-difluoro-14-{5-[2-(3-fluoroazetidin-1-yl)ethyl]-2-oxo-4-(trifluoromethyl)pyridin-1-yl}-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (200 mg, 0.26 mmol, 1.00 equiv.) and Zn(CN)₂ (62 mg, 0.52 mmol, 2.00 equiv.) in DMF (10 mL) were added XPhos Pd G₃ (22 mg, 0.026 mmol, 0.10 equiv.) and XPhos (25 mg, 0.052 mmol, 0.20 equiv.) at room temperature under nitrogen atmosphere. The resulting mixture was stirred for 2 h at 100° C. under nitrogen atmosphere and then cooled to room temperature. The resulting mixture was filtered, and the filter cake was washed with MeCN (3×10 mL). The filtrate was concentrated under reduced pressure. The residue was purified by reversed-phase flash chromatography (C18 gel; mobile phase, MeCN in water (0.1% NH₃·H₂O), 40% to 60% gradient in 10 min; detector, UV 220 nm) to provide ethyl 2-[(14S,17S)-5-cyano-12,19-difluoro-14-{5-[2-(3-fluoroazetidin-1-yl)ethyl]-2-oxo-4-(trifluoromethyl)pyridin-1-yl}-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (75 mg, 38.0%) as a white solid. LC-MS: (ES+H, m/z) [M+H]⁺=753.3. ¹H NMR (400 MHz, CD₃OD) δ 7.96 (d, J=1.6 Hz, 1H), 7.66-7.54 (m, 2H), 7.33 (s, 1H), 7.22 (t, J=9.0 Hz, 1H), 7.07-6.99 (m, 1H), 6.83 (d, J=23.5 Hz, 2H), 6.46 (dd, J=6.2, 2.2 Hz, 1H), 6.07 (dd, J=6.0, 3.0 Hz, 1H), 5.70 (t, J=7.9 Hz, 1H), 4.98 (p, J=5.2 Hz, 1H), 4.11 (qq, J=6.8, 3.6 Hz, 2H), 3.51-3.35 (m, 2H), 3.12-2.96 (m, 2H), 2.87 (dd, J=15.5, 8.2 Hz, 1H), 2.74 (dd, J=15.5, 7.6 Hz, 1H), 2.64-2.40 (m, 4H), 2.36 (s, 3H), 2.04 (s, 3H), 1.19 (t, J=7.1 Hz, 3H). ¹⁹F NMR (377 MHz, CD₃OD) δ −65.02, −122.20, −125.32, −180.21.

### Step 4: Preparation of [(14S,17R*)-5-cyano-12,19-difluoro-14-{5-[2-(3-fluoroazetidin-1-yl)ethyl]-2-oxo-4-(trifluoromethyl)pyridin-1-yl}-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid

To a stirred mixture of ethyl 2-[(14S,17S)-5-cyano-12,19-difluoro-14-{5-[2-(3-fluoroazetidin-1-yl)ethyl]-2-oxo-4-(trifluoromethyl)pyridin-1-yl}-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (70 mg, 0.093 mmol, 1.00 equiv.) in THF (5 mL) was added LiOH·H₂O (12 mg, 0.28 mmol, 3.00 equiv.) in H₂O (1 mL) at room temperature under nitrogen atmosphere. The resulting mixture was stirred for 2 h at room temperature and then acidified to pH 5 with HCl (aq. 1 N) and concentrated. The crude product was purified by Prep-HPLC (Column: Xbridge Phenyl OBD Column, 19*150 mm, 5 μm; Mobile Phase A: water (10 mmol/L NH₄HCO₃+0.05% NH₃H₂0, Mobile Phase B: MeCN; Flow rate: 60 mL/min; Gradient: 25% B to 35% B in 10 min; Wavelength: 254 nm/220 nm; RT1 (min) 10.2) to afford [(14S,17S)-5-cyano-12,19-difluoro-14-{5-[2-(3-fluoroazetidin-1-yl)ethyl]-2-oxo-4-(trifluoromethyl)pyridin-1-yl}-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid (26.2 mg, 38.9%) as a white solid. LC-MS: (ES+H, m/z) [M+H]⁺=725.25. ¹H NMR (400 MHz, CD₃OD) δ 7.95 (d, J=1.6 Hz, 1H), 7.66-7.54 (m, 2H), 7.32 (s, 1H), 7.20 (t, J=9.0 Hz, 1H), 7.05-6.96 (m, 1H), 6.86 (d, J=7.1 Hz, 2H), 6.49 (dd, J=6.1, 2.2 Hz, 1H), 6.08 (dd, J=6.1, 3.0 Hz, 1H), 5.70 (t, J=7.5 Hz, 1H), 5.07-4.88 (m, 1H), 3.65-3.47 (m, 2H), 3.25-3.11 (m, 2H), 2.82-2.58 (m, 4H), 2.55-2.42 (m, 2H), 2.35 (s, 3H), 2.06 (s, 3H). ¹⁹F NMR (377 MHz, CD₃OD) δ −65.00, −122.13, −125.30, −180.48.

### Example 47

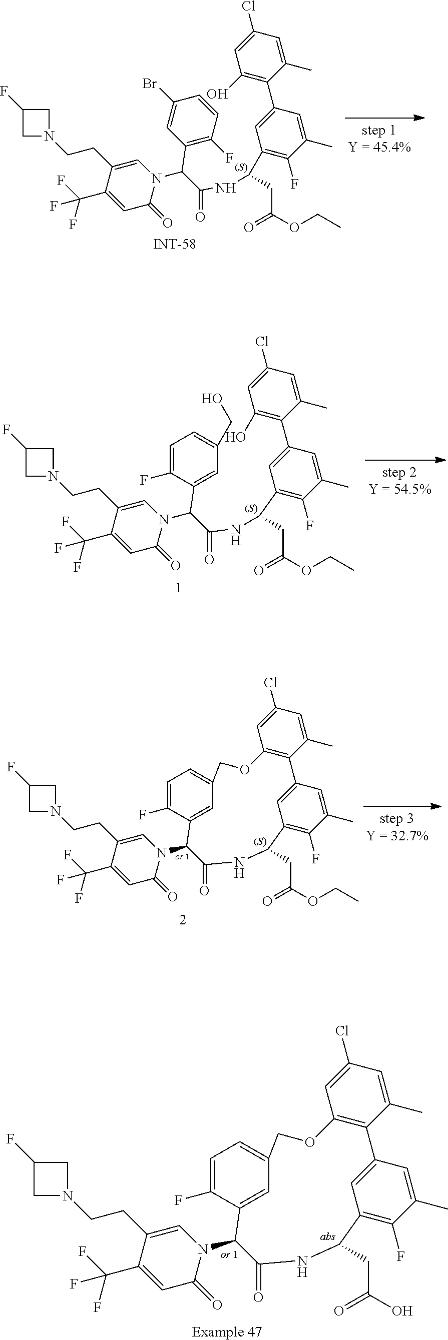

### Step 1: Preparation of ethyl (3S)-3-(4′-chloro-4-fluoro-2′-hydroxy-5,6′-dimethyl-[1,1′-biphenyl]-3-yl)-3-(2-(2-fluoro-5-(hydroxymethyl)phenyl)-2-(5-(2-(3-fluoroazetidin-1-yl)ethyl)-2-oxo-4-(trifluoromethyl)pyridin-1(2H)-yl)acetamido)propanoate (Mixture)

A mixture of ethyl (3S)-3-[2-(5-bromo-2-fluorophenyl)-2-{5-[2-(3-fluoroazetidin-1-yl)ethyl]-2-oxo-4-(trifluoromethyl)pyridin-1-yl}acetamido]-3-{4′-chloro-4-fluoro-2′-hydroxy-5,6′-dimethyl-[1,1′-biphenyl]-3-yl}propanoate (420 mg, 0.50 mmol, 1.00 equiv.), (tributylstannyl)methanol (319 mg, 0.99 mmol, 2.00 equiv.) and dicyclohexyl[2′,4′,6′-tris(propan-2-yl)-[1,1′-biphenyl]-2-yl]phosphane; {2′-amino-[1,1′-biphenyl]-2-yl}(chloro)palladium (39 mg, 0.05 mmol, 0.10 equiv.) in 1,4-dioxane (6 mL) was stirred for 4 h at 90° C. under nitrogen atmosphere. The reaction was then cooled to room temperature and concentrated. The residue was purified by reversed-phase flash chromatography (C18 gel; mobile phase, MeCN in water (0.1% NH₃·H₂O), 30% to 60% gradient in 10 min; detector, UV 254 nm) to provide ethyl (3S)-3-(4′-chloro-4-fluoro-2′-hydroxy-5,6′-dimethyl-[1,1′-biphenyl]-3-yl)-3-(2-(2-fluoro-5-(hydroxymethyl)phenyl)-2-(5-(2-(3-fluoroazetidin-1-yl)ethyl)-2-oxo-4-(trifluoromethyl)pyridin-1(2H)-yl)acetamido)propanoate (180 mg, 45.4%, mixture) as a white solid. LC-MS: (ES+H, m/z) [M+H]⁺=794.3.

### Step 2: Preparation of ethyl 2-((9S)-2⁴-chloro-1⁴,5⁴-difluoro-6-(5-(2-(3-fluoroazetidin-1-yl)ethyl)-2-oxo-4-(trifluoromethyl)pyridin-1(2H)-yl)-1⁵,2⁶-dimethyl-7-oxo-3-oxa-8-aza-1,5(1,3),2(1,2)-tribenzenacyclononaphane-9-yl)acetate

A mixture of ethyl (3S)-3-{4′-chloro-4-fluoro-2′-hydroxy-5,6′-dimethyl-[1,1′-biphenyl]-3-yl}-3-{2-[2-fluoro-5-(hydroxymethyl)phenyl]-2-{5-[2-(3-fluoroazetidin-1-yl)ethyl]-2-oxo-4-(trifluoromethyl)pyridin-1-yl}acetamido}propanoate (180 mg, 0.22 mmol, 1.00 equiv.) and 2-(tributyl-lambda5-phosphanylidene)acetonitrile (273 mg, 1.13 mmol, 5.00 equiv.) in toluene (3 mL) was stirred overnight at 100° C. under nitrogen atmosphere. The reaction was then cooled to room temperature and concentrated. The residue was purified by reversed-phase flash chromatography (C18 gel; mobile phase, MeCN in water (0.1% NH₃·H₂O), 30% to 60% gradient in 10 min; detector, UV 254 nm) to provide ethyl 2-((9S)-24-chloro-14,54-difluoro-6-(5-(2-(3-fluoroazetidin-1-yl)ethyl)-2-oxo-4-(trifluoromethyl)pyridin-1(2H)-yl)-15,26-dimethyl-7-oxo-3-oxa-8-aza-1,5(1,3),2(1,2)-tribenzenacyclononaphane-9-yl)acetate (96 mg, 54.5%) as a white solid. LC-MS: (ES+H, m/z) [M+H]⁺=776.3.

### Step 3: Preparation of 2-((6S,9S)-2⁴-chloro-1⁴,5⁴-difluoro-6-(5-(2-(3-fluoroazetidin-1-yl)ethyl)-2-oxo-4-(trifluoromethyl)pyridin-1(2H)-yl)-1⁵,2⁶-dimethyl-7-oxo-3-oxa-8-aza-1,5(1,3),2(1,2)-tribenzenacyclononaphane-9-yl)acetic acid

A mixture of ethyl 2-[(15S,18S)-5-chloro-13,20-difluoro-15-{5-[2-(3-fluoroazetidin-1-yl)ethyl]-2-oxo-4-(trifluoromethyl)pyridin-1-yl}-3,21-dimethyl-16-oxo-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetate (30 mg, 0.03 mmol, 1.00 equiv.) and LiOH·H₂O (8 mg, 0.19 mmol, 5.00 equiv.) in THF/H₂O (1 mL, 5/1) was stirred for 2 h at 40° C. under nitrogen atmosphere and then acidified to pH 5 with HCl (aq. 1N) and concentrated. The crude product was purified by Prep-HPLC (Column: XBridge Prep OBD RP18 Column, 30*150 mm, 5 μm; Mobile Phase A: water (10 mmol/L NH₄HCO₃), Mobile Phase B: MeCN; Flow rate: 60 mL/min; Gradient: 30% B to 46% B in 8 min; Wavelength: 254 nm/220 nm; RT1 (min) 9.57) to afford 2-((6S,9S)-24-chloro-14,54-difluoro-6-(5-(2-(3-fluoroazetidin-1-yl)ethyl)-2-oxo-4-(trifluoromethyl)pyridin-1(2H)-yl)-15,26-dimethyl-7-oxo-3-oxa-8-aza-1,5(1,3),2(1,2)-tribenzenacyclononaphane-9-yl)acetic acid (10.5 mg, 32.7%) as a white solid. LC-MS: (ES+H, m/z) [M+H]⁺=748.20. ¹H NMR (300 MHz, CD₃OD) δ 7.53 (d, J=6.6 Hz, 1H), 7.34 (d, J=7.2 Hz, 1H), 7.26-7.14 (m, 3H), 7.10 (s, 1H), 6.98 (d, J=6.8 Hz, 1H), 6.89 (s, 2H), 6.70 (d, J=6.3 Hz, 1H), 5.75 (t, J=7.1 Hz, 1H), 5.34 (d, J=13.3 Hz, 1H), 5.06 (d, J=13.3 Hz, 1H), 5.12-4.80 (m, 1H), 3.63-3.47 (m, 2H), 3.24-3.10 (m, 2H), 2.76-2.58 (m, 4H), 2.53-2.42 (m, 2H), 2.34 (s, 3H), 2.00 (s, 3H). ¹⁹F NMR (282 MHz, CD₃OD) δ −64.44, −117.94, −127.61, −180.48.

### Example 48

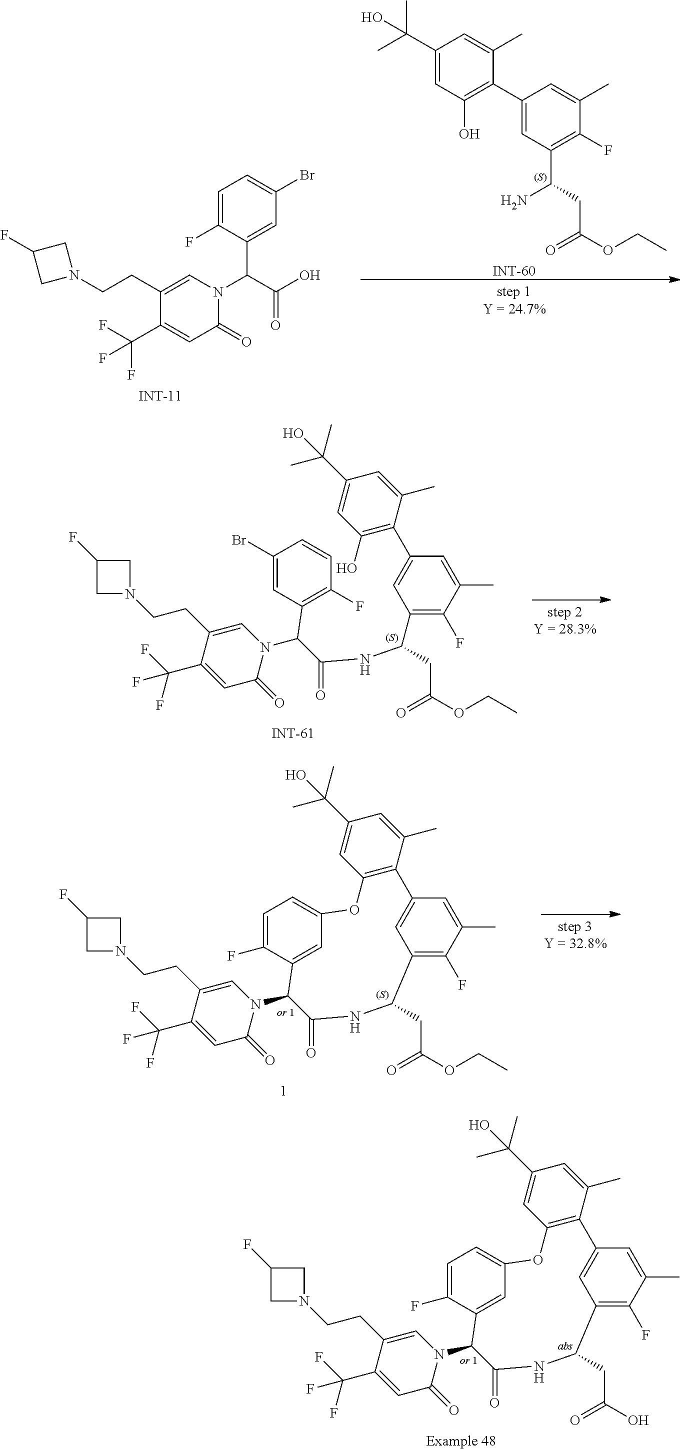

### Step 1: Preparation of ethyl (3S)-3-[2-(5-bromo-2-fluorophenyl)-2-{5-[2-(3-fluoroazetidin-1-yl)ethyl]-2-oxo-4-(trifluoromethyl)pyridin-1-yl}acetamido]-3-[4-fluoro-2′-hydroxy-4′-(2-hydroxypropan-2-yl)-5,6′-dimethyl-[1,1′-biphenyl]-3-yl]propanoate (Mixture)

To a stirred solution of (5-bromo-2-fluorophenyl)({5-[2-(3-fluoroazetidin-1-yl)ethyl]-2-oxo-4-(trifluoromethyl)pyridin-1-yl})acetic acid (168 mg, 0.34 mmol, 1.00 equiv.) and HOBT (59 mg, 0.44 mmol, 1.30 equiv.) in MeCN (2.5 mL) was added EDCI (97 mg, 0.51 mmol, 1.50 equiv.) in portions at room temperature under nitrogen atmosphere. The resulting mixture was stirred for 1 h at 40° C. under nitrogen atmosphere and then treated with ethyl (3S)-3-amino-3-[4-fluoro-2′-hydroxy-4′-(2-hydroxypropan-2-yl)-5,6′-dimethyl-[1,1′-biphenyl]-3-yl]propanoate hydrochloride (120 mg, 0.28 mmol, 1.00 equiv.) in MeCN (0.5 mL) dropwise at room temperature. The resulting mixture was stirred overnight at room temperature and then diluted with water (15 mL) and extracted with CH₂Cl₂ (3×30 mL). The combined organic layers were dried over anhydrous Na₂SO₄, filtered, and concentrated. The residue was purified by Prep-TLC (CH₂Cl₂/MeOH 15:1) to afford ethyl (3S)-3-[2-(5-bromo-2-fluorophenyl)-2-{5-[2-(3-fluoroazetidin-1-yl)ethyl]-2-oxo-4-(trifluoromethyl)pyridin-1-yl}acetamido]-3-[4-fluoro-2′-hydroxy-4′-(2-hydroxypropan-2-yl)-5,6′-dimethyl-[1,1′-biphenyl]-3-yl]propanoate (73 mg, 24.7%) as an off-white solid. LC-MS: (ES+H, m/z) [M+H]⁺=868.25.

### Step 2: Preparation of ethyl 2-[(14S,17S)-12,19-difluoro-14-{5-[2-(3-fluoroazetidin-1-yl)ethyl]-2-oxo-4-(trifluoromethyl)pyridin-1-yl}-5-(2-hydroxypropan-2-yl)-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate

To a stirred solution of ethyl (3S)-3-[2-(5-bromo-2-fluorophenyl)-2-{5-[2-(3-fluoroazetidin-1-yl)ethyl]-2-oxo-4-(trifluoromethyl)pyridin-1-yl}acetamido]-3-[4-fluoro-2′-hydroxy-4′-(2-hydroxypropan-2-yl)-5,6′-dimethyl-[1,1′-biphenyl]-3-yl]propanoate (70 mg, 0.08 mmol, 1.00 equiv., mixture) and (1R,2R)—N1,N2-dimethylcyclohexane-1,2-diamine (11 mg, 0.08 mmol, 1.00 equiv.) in 1,4-dioxane (2 mL) were added CuI (7 mg, 0.04 mmol, 0.50 equiv.) and K₂CO₃ (22 mg, 0.16 mmol, 2.00 equiv.) in portions at room temperature under nitrogen atmosphere. The resulting mixture was stirred overnight at 100° C. under nitrogen atmosphere and then cooled to room temperature and concentrated. The residue was purified by Prep-TLC (CH₂Cl₂/MeOH 15:1), to afford ethyl 2-[(14S,17S)-12,19-difluoro-14-{5-[2-(3-fluoroazetidin-1-yl)ethyl]-2-oxo-4-(trifluoromethyl)pyridin-1-yl}-5-(2-hydroxypropan-2-yl)-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (18 mg, 28.3%) as a light yellow solid. LC-MS: (ES+H, m/z) [M+H]⁺=786.35.

### Step 3: Preparation of [(14S,17S)-12,19-difluoro-14-{5-[2-(3-fluoroazetidin-1-yl)ethyl]-2-oxo-4-(trifluoromethyl)pyridin-1-yl}-5-(2-hydroxypropan-2-yl)-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid

To a stirred solution of ethyl 2-[(14S,17S)-12,19-difluoro-14-{5-[2-(3-fluoroazetidin-1-yl)ethyl]-2-oxo-4-(trifluoromethyl)pyridin-1-yl}-5-(2-hydroxypropan-2-yl)-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (18 mg, 0.02 mmol, 1.00 equiv.) in THF (1 mL) was added LiOH·H₂O (2 mg, 0.04 mmol, 2.00 equiv.) in water (0.5 mL) dropwise at 0° C. under nitrogen atmosphere. The resulting mixture was stirred for 2 h at room temperature and then concentrated. The crude product was purified by Prep-HPLC (Column: XSelect CSH Fluoro Phenyl, 30*150 mm, 5 μm; Mobile Phase A: water (0.1% HCOOH), Mobile Phase B: MeCN; Flow rate: 60 mL/min; Gradient: 9% B to 29% B in 10 min; Wavelength: 254 nm/220 nm; RT1 (min) 10.6), to afford [(14S,17S)-12,19-difluoro-14-{5-[2-(3-fluoroazetidin-1-yl)ethyl]-2-oxo-4-(trifluoromethyl)pyridin-1-yl}-5-(2-hydroxypropan-2-yl)-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid (5.7 mg, 32.8%) as a white solid. LC-MS: (ES+H, m/z) [M+H]⁺=758.20. ¹H NMR (400 MHz, CD₃OD) δ 7.60-7.51 (m, 2H), 7.36 (s, 1H), 7.25 (d, J=1.7 Hz, 1H), 7.17 (t, J=9.0 Hz, 1H), 6.96 (d, J=7.0 Hz, 1H), 6.86 (d, J=5.2 Hz, 2H), 6.45 (d, J=6.2 Hz, 1H), 6.13-6.09 (m, 1H), 5.69 (t, J=7.5 Hz, 1H), 5.06-4.90 (m, 1H), 3.60 (brs, 2H), 3.30-3.29 (m, 2H), 2.72-2.61 (m, 4H), 2.49 (d, J=6.2 Hz, 2H), 2.34 (d, J=1.8 Hz, 3H), 2.01 (s, 3H), 1.60 (s, 6H). ¹⁹F NMR (377 MHz, CD₃OD) δ −64.99, −123.25, −126.86, −180.49.

### Example 49

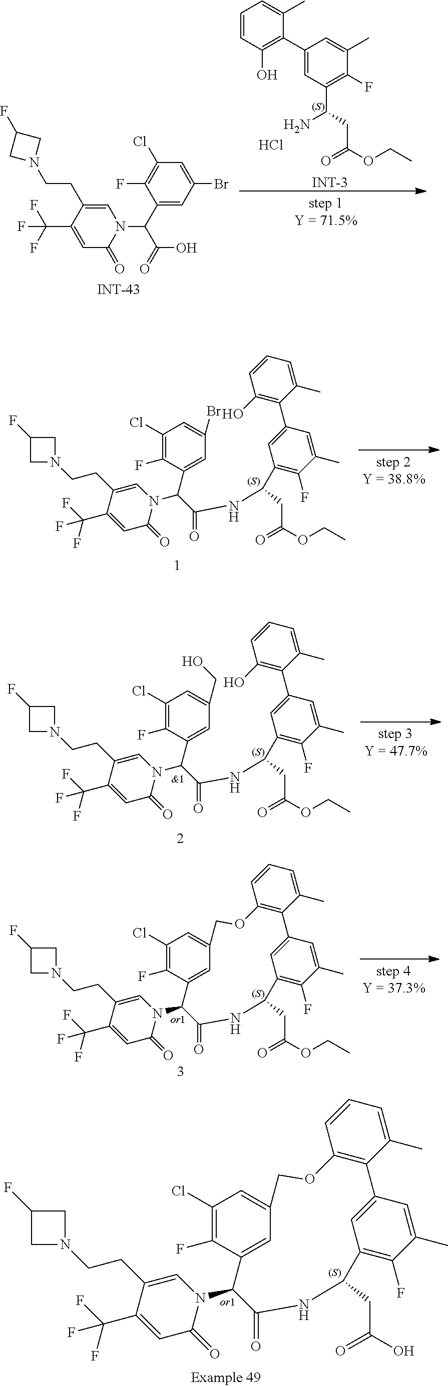

### Step 1: Preparation of ethyl (3S)-3-(2-(5-bromo-3-chloro-2-fluorophenyl)-2-(5-(2-(3-fluoroazetidin-1-yl)ethyl)-2-oxo-4-(trifluoromethyl)pyridin-1(2H)-yl)acetamido)-3-(4-fluoro-2′-hydroxy-5,6′-dimethyl-[1,1′-biphenyl]-3-yl)propanoate

A mixture of ethyl (S)-3-amino-3-(4-fluoro-2′-hydroxy-5,6′-dimethyl-[1,1′-biphenyl]-3-yl)propanoate hydrochloride (360 mg, 0.98 mmol, 1.44 equiv.) and NEt₃ (413 mg, 4.08 mmol, 6.00 equiv.) in MeCN (5 mL) was stirred for 30 min at 30° C. under nitrogen atmosphere. The resulting mixture was concentrated under reduced to provide ethyl (S)-3-amino-3-(4-fluoro-2′-hydroxy-5,6′-dimethyl-[1,1′-biphenyl]-3-yl)propanoate. To a stirred mixture of 2-(5-bromo-3-chloro-2-fluorophenyl)-2-(5-(2-(3-fluoroazetidin-1-yl)ethyl)-2-oxo-4-(trifluoromethyl)pyridin-1(2H)-yl)acetic acid (360 mg, 0.68 mmol, 1.00 equiv.) in MeCN (5 mL) was added EDCI (195 mg, 1.02 mmol, 1.50 equiv.) and HOBT (119 mg, 0.88 mmol, 1.30 equiv.) at room temperature. The resulting mixture was stirred for 1 h at 30° C. under nitrogen atmosphere and then was treated with ethyl (S)-3-amino-3-(4-fluoro-2′-hydroxy-5,6′-dimethyl-[1,1′-biphenyl]-3-yl)propanoate in MeCN (5 mL) dropwise at room temperature. The resulting mixture was stirred overnight at 40° C. and then cooled to room temperature and concentrated. The residue was purified by reversed-phase flash chromatography (C18 gel; mobile phase, MeCN in water (0.1% NH₃·H₂O), 45% to 70% gradient in 30 min; detector, UV 220 nm) to provide ethyl (3S)-3-(2-(5-bromo-3-chloro-2-fluorophenyl)-2-(5-(2-(3-fluoroazetidin-1-yl)ethyl)-2-oxo-4-(trifluoromethyl)pyridin-1(2H)-yl)acetamido)-3-(4-fluoro-2′-hydroxy-5,6′-dimethyl-[1,1′-biphenyl]-3-yl)propanoate (410 mg, 71.5%) as a yellow solid. LC-MS: (ES+H, m/z) [M+H]⁺=842.0.

### Step 2: Preparation of ethyl (3S)-3-(2-(3-chloro-2-fluoro-5-(hydroxymethyl)phenyl)-2-(5-(2-(3-fluoroazetidin-1-yl)ethyl)-2-oxo-4-(trifluoromethyl)pyridin-1(2H)-yl)acetamido)-3-(4-fluoro-2′-hydroxy-5,6′-dimethyl-[1,1′-biphenyl]-3-yl)propanoate

To a stirred solution of ethyl (3S)-3-(2-(5-bromo-3-chloro-2-fluorophenyl)-2-(5-(2-(3-fluoroazetidin-1-yl)ethyl)-2-oxo-4-(trifluoromethyl)pyridin-1(2H)-yl)acetamido)-3-(4-fluoro-2′-hydroxy-5,6′-dimethyl-[1,1′-biphenyl]-3-yl)propanoate (410 mg, 0.49 mmol, 1.00 equiv.) in dioxane (20 mL) was added (tributylstannyl)methanol (312 mg, 0.97 mmol, 2.00 equiv.) and dicyclohexyl[2′,4′,6′-tris(propan-2-yl)-[1,1′-biphenyl]-2-yl]phosphane; {2′-amino-[1,1′-biphenyl]-2-yl}(chloro)palladium (38 mg, 0.05 mmol, 0.10 equiv.). The resulting mixture was stirred for 4 h at 90° C. under nitrogen atmosphere and then cooled to room temperature. The resulting mixture was diluted with EtOAc (100 mL) and washed with water (2×20 mL), dried over anhydrous Na₂SO₄, filtered, and concentrated. The residue was purified by reversed-phase flash chromatography (C18 gel; mobile phase, MeCN in water (0.1% NH₃·H₂O), 40% to 70% gradient in 30 min; detector, UV 220 nm) to provide ethyl (3S)-3-(2-(3-chloro-2-fluoro-5-(hydroxymethyl)phenyl)-2-(5-(2-(3-fluoroazetidin-1-yl)ethyl)-2-oxo-4-(trifluoromethyl)pyridin-1(2H)-yl)acetamido)-3-(4-fluoro-2′-hydroxy-5,6′-dimethyl-[1,1′-biphenyl]-3-yl)propanoate (150 mg, 38.8%) as a yellow solid. LC-MS: (ES+H, m/z) [M+H]⁺=794.3.

### Step 3: Preparation of ethyl 2-[(15S,18S)-12-chloro-13,20-difluoro-15-{5-[2-(3-fluoroazetidin-1-yl)ethyl]-2-oxo-4-(trifluoromethyl)pyridin-1-yl}-3,21-dimethyl-16-oxo-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetate

A mixture of ethyl (3S)-3-{2-[3-chloro-2-fluoro-5-(hydroxymethyl)phenyl]-2-{5-[2-(3-fluoroazetidin-1-yl)ethyl]-2-oxo-4-(trifluoromethyl)pyridin-1-yl}acetamido}-3-{4-fluoro-2′-hydroxy-5,6′-dimethyl-[1,1′-biphenyl]-3-yl}propanoate (150 mg, 0.19 mmol, 1.00 equiv.) and 2-(tributyl-lambda5-phosphanylidene)acetonitrile (228 mg, 0.95 mmol, 5.00 equiv.) in PhCH₃ (5 mL) was stirred overnight at 100° C. under nitrogen atmosphere and then cooled to room temperature and concentrated. The residue was purified by reversed-phase flash chromatography (C18 gel; mobile phase, MeCN in water (0.1% NH₃·H₂O), 40% to 70% gradient in 30 min; detector, UV 220 nm) to provide ethyl 2-[(15S,18S)-12-chloro-13,20-difluoro-15-{5-[2-(3-fluoroazetidin-1-yl)ethyl]-2-oxo-4-(trifluoromethyl)pyridin-1-yl}-3,21-dimethyl-16-oxo-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetate (70 mg, 47.7%) as a yellow solid. LC-MS: (ES+H, m/z) [M+H]⁺=776.4. ¹1H NMR (400 MHz, CD₃OD) δ 7.66 (dd, J=6.6, 2.1 Hz, 1H), 7.27-7.11 (m, 4H), 7.04 (s, 1H), 6.97 (d, J=7.4 Hz, 1H), 6.85 (d, J=7.5 Hz, 1H), 6.64 (dd, J=6.7, 2.2 Hz, 1H), 5.75 (dd, J=9.3, 5.8 Hz, 1H), 5.31 (d, J=13.3 Hz, 1H), 4.98 (dd, J=11.9, 6.3 Hz, 2H), 4.14 (ddp, J=10.7, 7.4, 3.7 Hz, 3H), 3.39 (dq, J=13.9, 7.8, 6.6 Hz, 3H), 3.03 (tdd, J=22.7, 9.5, 4.7 Hz, 3H), 2.78 (qd, J=15.6, 7.4 Hz, 2H), 2.57-2.44 (m, 3H), 2.32 (d, J=2.0 Hz, 3H), 1.98 (s, 2H), 1.20 (td, J=7.1, 1.5 Hz, 3H).

### Step 4: Preparation of [(15S,18S)-12-chloro-13,20-difluoro-15-{5-[2-(3-fluoroazetidin-1-yl)ethyl]-2-oxo-4-(trifluoromethyl)pyridin-1-yl}-3,21-dimethyl-16-oxo-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetic acid

A mixture of ethyl 2-[(15S,18S)-12-chloro-13,20-difluoro-15-{5-[2-(3-fluoroazetidin-1-yl)ethyl]-2-oxo-4-(trifluoromethyl)pyridin-1-yl}-3,21-dimethyl-16-oxo-8-oxa-17-azatetracyclo [17.3.1.1\hat{ }{10,14}.0\hat{ }{2,7}]tetracosa-1(23),2(7),3,5,10,12,14(24),19,21-nonaen-18-yl]acetate (90 mg, 0.12 mmol, 1.00 equiv.) and LiOH·H₂O (19 mg, 0.46 mmol, 4.00 equiv.) in THF (2 mL) and H₂O (0.4 mL) was stirred for 2 h at 30° C. under nitrogen atmosphere and then cooled to room temperature. The mixture was acidified to pH 5 with HCl (aq. 1 N) and then concentrated. The crude product was purified by Prep-HPLC (Column: XBridge Prep OBD C18 Column, 30*150 mm, 10 μm; Mobile Phase A: water (10 mmol/L NH₄HCO₃+0.050% NH₃H₂O), Mobile Phase B: MeCN; Flow rate: 60 mL/min; Gradient: 30% B to 45% B in 8 min; Wavelength: 254 nm/220 nm; RT1 (min) 9.33) to afford [(15S,18S)-12-chloro-13,20-difluoro-15-{5-[2-(3-fluoroazetidin-1-yl)ethyl]-2-oxo-4-(trifluoromethyl)pyridin-1-yl}-3,21-dimethyl-16-oxo-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetic acid (33.0 mg, 37.3%) as a white solid. LC-MS: (ES+H, m/z) [M+H]⁺=748.30. ¹H NMR (400 MHz, CD₃OD) δ 7.66-7.60 (m, 1H), 7.26 (d, J=6.3 Hz, 1H), 7.23-7.16 (m, 2H), 7.16-7.06 (m, 2H), 6.99-6.92 (m, 1H), 6.90-6.82 (m, 2H), 6.72-6.66 (m, 1H), 5.72 (dd, J=8.6, 5.6 Hz, 1H), 5.30 (d, J=13.4 Hz, 1H), 5.06-4.87 (m, 2H), 3.59-3.45 (m, 2H), 3.26-3.13 (m, 2H), 2.77-2.56 (m, 4H), 2.47 (t, J=7.0 Hz, 2H), 2.31 (s, 3H), 1.99 (s, 3H). ⁹F NMR (377 MHz, CD₃OD) δ −64.945, −119.938, −128.148, −180.364.

### Example 50

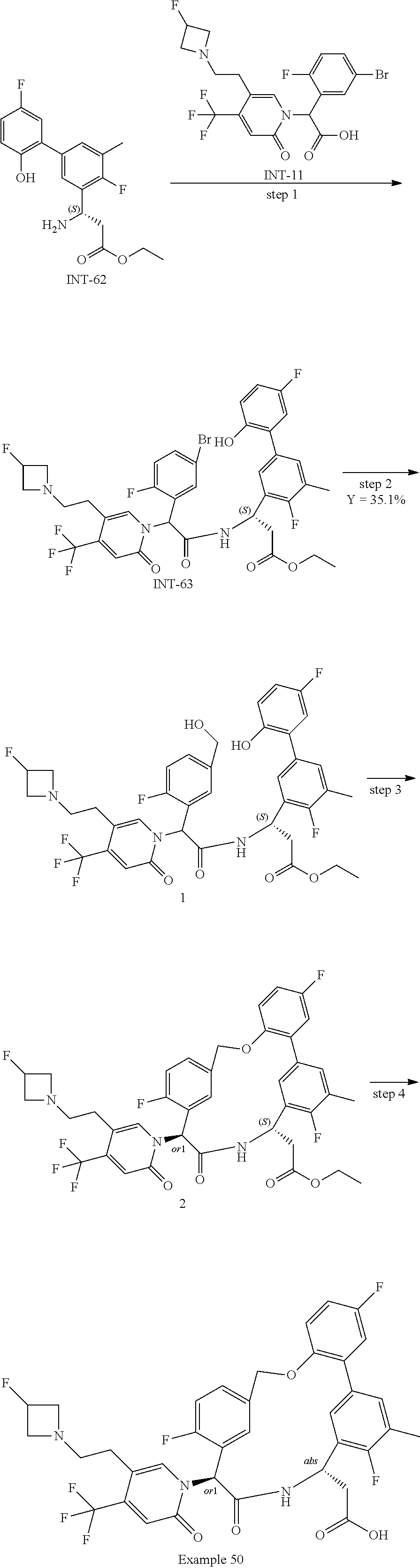

### Step 1: Preparation of ethyl (3S)-3-[2-(5-bromo-2-fluorophenyl)-2-{5-[2-(3-fluoroazetidin-1-yl)ethyl]-2-oxo-4-(trifluoromethyl)pyridin-1-yl}acetamido]-3-{4,5′-difluoro-2′-hydroxy-5-methyl-[1,1′-biphenyl]-3-yl}propanoate

To a stirred solution of ethyl (3S)-3-amino-3-{4,5′-difluoro-2′-hydroxy-5-methyl-[1,1′-biphenyl]-3-yl}propanoate (271 mg, 0.81 mmol, 1.00 equiv.) and (5-bromo-2-fluorophenyl)({5-[2-(3-fluoroazetidin-1-yl)ethyl]-2-oxo-4-(trifluoromethyl)pyridin-1-yl})acetic acid (400 mg, 0.81 mmol, 1.00 equiv.) in CH₂Cl₂ (7 mL) were added DIEA (522 mg, 4.04 mmol, 5.00 equiv.) and HATU (369 mg, 0.97 mmol, 1.20 equiv.) in portions at 0° C. The resulting mixture was stirred overnight at 40° C. under nitrogen atmosphere and then quenched by the addition of water/ice (50 mL) at room temperature. The resulting mixture was extracted with EtOAc (3×30 mL) and the combined organic layers were washed with brine (1×30 mL), dried over anhydrous Na₂SO₄, filtered, and concentrated. The residue was purified by silica gel column chromatography (petroleum ether/EtOAc (0% to 60% gradient in 25 min) to afford ethyl (3S)-3-[2-(5-bromo-2-fluorophenyl)-2-{5-[2-(3-fluoroazetidin-1-yl)ethyl]-2-oxo-4-(trifluoromethyl)pyridin-1-yl}acetamido]-3-{4,5′-difluoro-2′-hydroxy-5-methyl-[1,1′-biphenyl]-3-yl}propanoate (385 mg, 58.6%) as a yellow solid. LC-MS: (ES+H, m/z) [M+H]⁺=811.9. ¹H NMR (400 MHz, CDCl₃) δ 7.89-7.73 (m, 1H), 7.66 (d, J=11.8 Hz, 1H), 7.41 (s, 1H), 7.16 (d, J=34.5 Hz, 1H), 7.08-7.00 (m, 3H), 6.94-6.89 (m, 4H), 6.69 (s, 1H), 5.58 (dd, J=29.1, 6.9 Hz, 1H), 5.30 (s, 1H), 4.09 (dd, J=15.3, 8.2 Hz, 3H), 2.97-2.86 (m, 2H), 2.80 (s, 2H), 2.36-2.30 (m, 4H), 2.05 (s, 1H), 1.25 (d, J=7.1 Hz, 2H), 1.16-1.14 (m, 3H).

### Step 2: Preparation of ethyl (3S)-3-{4,5′-difluoro-2′-hydroxy-5-methyl-[1,1′-biphenyl]-3-yl}-3-{2-[2-fluoro-5-(hydroxymethyl)phenyl]-2-{5-[2-(3-fluoroazetidin-1-yl)ethyl]-2-oxo-4-(trifluoromethyl) pyridin-1-yl}acetamido}propanoate

To a stirred solution of ethyl (3S)-3-[2-(5-bromo-2-fluorophenyl)-2-{5-[2-(3-fluoroazetidin-1-yl)ethyl]-2-oxo-4-(trifluoromethyl)pyridin-1-yl}acetamido]-3-{4,5′-difluoro-2′-hydroxy-5-methyl-[1,1′-biphenyl]-3-yl}propanoate (200 mg, 0.25 mmol, 1.00 equiv.) and (tributylstannyl)methanol (237 mg, 0.74 mmol, 3.00 equiv.) in dioxane (2.5 mL) was added dicyclohexyl[2′,4′,6′-tris(propan-2-yl)-[1,1′-biphenyl]-2-yl]phosphane; {2′-amino-[1,1′-biphenyl]-2-yl}(chloro)palladium (19 mg, 0.03 mmol, 0.10 equiv.) in portions at room temperature. The resulting mixture was stirred overnight at 80° C. under nitrogen atmosphere and then cooled to room temperature and concentrated. The residue was purified by Prep-TLC (CH₂Cl₂/MeOH 15:1, Rf=0.5) to afford ethyl (3S)-3-{4,5′-difluoro-2′-hydroxy-5-methyl-[1,1′-biphenyl]-3-yl}-3-{2-[2-fluoro-5-(hydroxymethyl)phenyl]-2-{5-[2-(3-fluoroazetidin-1-yl)ethyl]-2-oxo-4-(trifluoromethyl)pyridin-1-yl}acetamido}propanoate (66 mg, 35.1%, mixture) as a yellow solid. LC-MS: (ES+H, m/z) [M+H]⁺=764.1.

### Step 3: Preparation of ethyl 2-[(15S,18S)-4,13,20-trifluoro-15-{5-[2-(3-fluoroazetidin-1-yl)ethyl]-2-oxo-4-(trifluoromethyl)pyridin-1-yl}-21-methyl-16-oxo-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetate

A solution of ethyl (3S)-3-{4,5′-difluoro-2′-hydroxy-5-methyl-[1,1′-biphenyl]-3-yl}-3-{2-[2-fluoro-5-(hydroxymethyl)phenyl]-2-{5-[2-(3-fluoroazetidin-1-yl)ethyl]-2-oxo-4-(trifluoromethyl)pyridin-1-yl}acetamido}propanoate (66 mg, 0.09 mmol, 1.00 equiv.) and 2-(tributyl-lambda5-phosphanylidene)acetonitrile (104 mg, 0.43 mmol, 5.00 equiv.) in toluene (2 mL) was stirred overnight at 100° C. under nitrogen atmosphere and then cooled to room temperature. The resulting mixture was concentrated under reduced pressure. The residue was purified by Prep-TLC (CH₂Cl₂/MeOH 15:1, Rf=0.5) to afford ethyl 2-[(15S,18S)-4,13,20-trifluoro-15-{5-[2-(3-fluoroazetidin-1-yl)ethyl]-2-oxo-4-(trifluoromethyl) 359yridine-1-yl}-21-methyl-16-oxo-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetate (33 mg, crude) as a yellow solid. LC-MS: (ES+H, m/z) [M+H]⁺=746.1.

### Step 4: Preparation of [(15S,18S)-4,13,20-trifluoro-15-{5-[2-(3-fluoroazetidin-1-yl)ethyl]-2-oxo-4-(trifluoromethyl)359yridine-1-yl}-21-methyl-16-oxo-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetic acid

A solution of ethyl 2-[(15S,18S)-4,13,20-trifluoro-15-{5-[2-(3-fluoroazetidin-1-yl)ethyl]-2-oxo-4-(trifluoromethyl)359yridine-1-yl}-21-methyl-16-oxo-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetate (33 mg, 0.04 mmol, 1.00 equiv.) and LiOH·H₂O (6 mg, 0.13 mmol, 3.00 equiv.) in THF (1.2 mL) and H₂O (0.3 mL) was stirred for 3 h at room temperature. The mixture was then acidified to pH 5 with HCl (aq. 1 N) and concentrated. The crude product was purified by Prep-HPLC (Column: XBridge Phenyl OBD Column 19*150 mm, 5 μm; Mobile Phase A: water (10 mmol/L NH₃HCO₃+0.05% NH₃H₂O), Mobile Phase B: MeCN; Flow rate: 60 mL/min; Gradient: 25% B to 40% B in 10 min; Wavelength: 254 nm/220 nm; RT1 (min) 9.65) to afford [(15S,18S)-4,13,20-trifluoro-15-{5-[2-(3-fluoroazetidin-1-yl)ethyl]-2-oxo-4-(trifluoromethyl)pyridin-1-yl}-21-methyl-16-oxo-8-oxa-17-azatetracyclo[17.3.1.1\hat{ }{10,14}.0\hat{ }{2,7}] tetracosa-1(23),2(7),3,5,10,12,14(24),19,21-nonaen-18-yl]acetic acid (6.4 mg, 10.2%) as a white solid. LC-MS: (ES+H, m/z) [M+H]⁺=718.20. ¹H NMR (400 MHz, CD₃OD) δ 7.71 (d, J=6.9 Hz, 1H), 7.57 (d, J=5.9 Hz, 1H), 7.54-7.45 (m, 1H), 7.26 7.14 m, 4H), 7.14-7.05 (m, 3H), 6.88 (s, 1H), 5.66 (t, J=7.2 Hz, 1H), 5.27 (d, J=12.6 Hz, 1H), 5.09 (d, J=12.6 Hz, 1H), 4.99-4.86 (m, 1H), 3.52-3.41 (m, 2H), 3.21-3.05 (m, 2H), 2.77-2.49 (m, 4H), 2.42 (q, J=7.0 Hz, 2H), 2.33 (s, 3H). ¹⁹F NMR (377 MHz, CD₃OD) δ −64.94, −118.73, −125.09, −180.64.

### Example 51

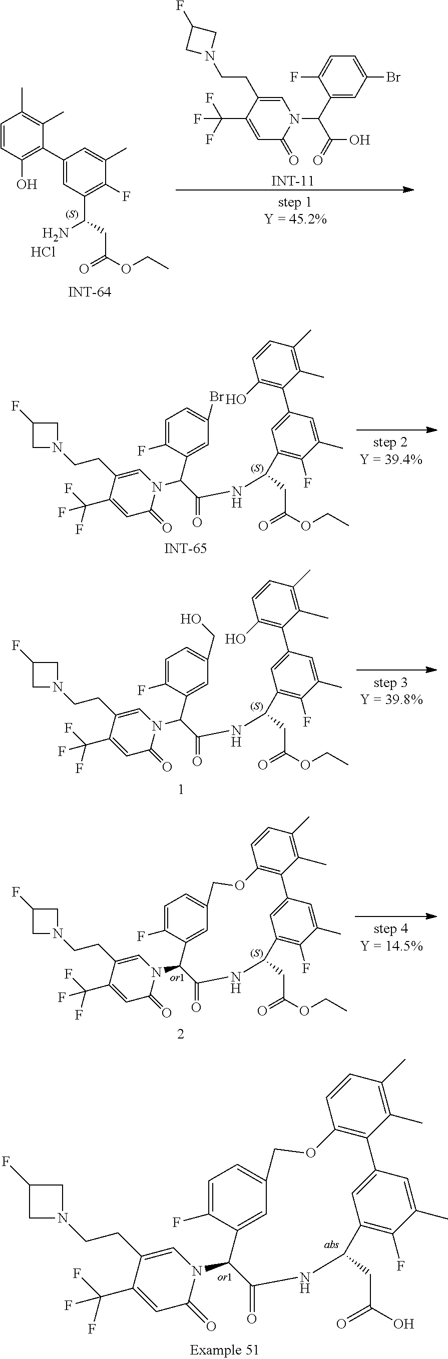

### Step 1: Preparation of ethyl (3S)-3-[2-(5-bromo-2-fluorophenyl)-2-{5-[2-(3-fluoroazetidin-1-yl)ethyl]-2-oxo-4-(trifluoromethyl)pyridin-1-yl}acetamido]-3-{4-fluoro-6′-hydroxy-2′,3′,5-trimethyl-[1,1′-biphenyl]-3-yl}propanoate (Mixture)

A mixture of (5-bromo-2-fluorophenyl)({5-[2-(3-fluoroazetidin-1-yl)ethyl]-2-oxo-4-(trifluoromethyl)pyridin-1-yl})acetic acid (400 mg, 0.81 mmol, 1.00 equiv.), EDCI (232 mg, 1.21 mmol, 1.50 equiv.) and HOBT (142 mg, 1.05 mmol, 1.30 equiv.) in MeCN (5 mL) was stirred for 1 h at 30° C. under nitrogen atmosphere to afford mixture 1. In a separate vial, ethyl (3S)-3-amino-3-{4-fluoro-6′-hydroxy-2′,3′,5-trimethyl-[1,1′-biphenyl]-3-yl}propanoate (399 mg, 1.16 mmol, 1.43 equiv.) and NEt₃ (490 mg, 4.85 mmol, 6.00 equiv.) in MeCN (5 mL) was stirred for 0.5 h at 30° C. under nitrogen atmosphere, then concentrated under reduced pressure and redissolved in MeCN (3 mL) to afford mixture 2. The mixture 2 was added to mixture 1 and was stirred for 3 h at 40° C. under nitrogen atmosphere. The reaction was then concentrated and the residue was purified by reversed-phase flash chromatography (C18 gel; mobile phase, MeCN in water (0.1% NH₃·H₂O), 50% to 70% gradient in 30 min; detector, UV 220 nm) to provide ethyl (3S)-3-[2-(5-bromo-2-fluorophenyl)-2-{5-[2-(3-fluoroazetidin-1-yl)ethyl]-2-oxo-4-(trifluoromethyl)pyridin-1-yl}acetamido]-3-{4-fluoro-6′-hydroxy-2′,3′,5-trimethyl-[1,1′-biphenyl]-3-yl}propanoate (300 mg, 45.2%) as a yellow oil. LC-MS: (ES+H, m/z) [M+H]⁺=824.2.

### Step 2: Preparation of ethyl (3S)-3-{2-[2-fluoro-5-(hydroxymethyl)phenyl]-2-{5-[2-(3-fluoroazetidin-1-yl)ethyl]-2-oxo-4-(trifluoromethyl)pyridin-1-yl}acetamido}-3-{4-fluoro-6′-hydroxy-2′,3′,5-trimethyl-[1,1′-biphenyl]-3-yl}propanoate (Mixture)

A mixture of ethyl (3S)-3-[2-(5-bromo-2-fluorophenyl)-2-{5-[2-(3-fluoroazetidin-1-yl)ethyl]-2-oxo-4-(trifluoromethyl)pyridin-1-yl}acetamido]-3-{4-fluoro-6′-hydroxy-2′,3′,5-trimethyl-[1,1′-biphenyl]-3-yl}propanoate (270 mg, 0.33 mmol, 1.00 equiv., mixture), (tributylstannyl)methanol (369 mg, 1.15 mmol, 3.50 equiv.) and 2nd Generation XPhos Precatalyst (82 mg, 0.10 mmol, 0.32 equiv.) in dioxane (10 mL) was stirred for 5 h at 90° C. under nitrogen atmosphere. The reaction was then cooled to room temperature and concentrated. The residue was purified by reversed-phase flash chromatography (C18 gel; mobile phase, MeCN in water (0.1% NH₃·H₂O), 50% to 80% gradient in 30 min; detector, UV 220 nm) to provide ethyl (3S)-3-{2-[2-fluoro-5-(hydroxymethyl)phenyl]-2-{5-[2-(3-fluoroazetidin-1-yl)ethyl]-2-oxo-4-(trifluoromethyl)pyridin-1-yl}acetamido}-3-{4-fluoro-6′-hydroxy-2′,3′,5-trimethyl-[1,1′-biphenyl]-3-yl}propanoate (100 mg, 39.4%, mixture) as a white solid. LC-MS: (ES+H, m/z) [M+H]⁺=774.3.

### Step 3: Preparation of ethyl 2-[(15S,18S)-13,20-difluoro-15-{5-[2-(3-fluoroazetidin-1-yl)ethyl]-2-oxo-4-(trifluoromethyl)pyridin-1-yl}-3,4,21-trimethyl-16-oxo-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetate

A mixture of ethyl (3S)-3-{2-[2-fluoro-5-(hydroxymethyl)phenyl]-2-{5-[2-(3-fluoroazetidin-1-yl)ethyl]-2-oxo-4-(trifluoromethyl)pyridin-1-yl}acetamido}-3-{4-fluoro-6′-hydroxy-2′,3′,5-trimethyl-[1,1′-biphenyl]-3-yl}propanoate (90 mg, 0.12 mmol, 1.00 equiv.) and 2-(tributyl-15-phosphaneylidene)acetonitrile (84 mg, 0.35 mmol, 3.00 equiv.) in toluene (10 mL) was stirred for 2 h at 100° C. under nitrogen atmosphere. The resulting mixture was concentrated under reduced pressure. The residue was purified by reversed-phase flash chromatography (C18 gel; mobile phase, MeCN in water (0.1% NH₃·H₂O), 60% to 80% gradient in 30 min; detector, UV 220 nm) to provide ethyl 2-[(15S,18S)-13,20-difluoro-15-{5-[2-(3-fluoroazetidin-1-yl)ethyl]-2-oxo-4-(trifluoromethyl)pyridin-1-yl}-3,4,21-trimethyl-16-oxo-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetate (35 mg, 39.8%) as a white solid. LC-MS: (ES+H, m/z) [M+H]⁺=756.2.

### Step 4: Preparation of [(15R*,18S)-13,20-difluoro-15-{5-[2-(3-fluoroazetidin-1-yl)ethyl]-2-oxo-4-(trifluoromethyl)pyridin-1-yl}-3,4,21-trimethyl-16-oxo-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetic acid

A mixture of ethyl 2-[(15S,18S)-13,20-difluoro-15-{5-[2-(3-fluoroazetidin-1-yl)ethyl]-2-oxo-4-(trifluoromethyl)pyridin-1-yl}-3,4,21-trimethyl-16-oxo-8-oxa-17-azatetracyclo [17.3.1.1\hat{ }{10,14}.0\hat{ }{2,7}]tetracosa-1(23),2(7),3,5,10,12,14(24),19,21-nonaen-18-yl]acetate (30 mg, 0.040 mmol, 1.00 equiv.) and LiOH·H₂O (8 mg, 0.20 mmol, 5.00 equiv.) in THF (1.5 mL) and H₂O (0.3 mL) was stirred for 2 h at 30° C. The mixture was acidified to pH 5 with HCl (aq. 1 M) and then concentrated. The crude product was purified by Prep-HPLC (Column: XSelect CSH Fluoro Phenyl, 30*150 mm, 5 μm; Mobile Phase A: water (0.1% HCOOH), Mobile Phase B: MeCN; Flow rate: 60 mL/min; Gradient: 17% B to 37% B in 10 min; Wavelength: 254 nm/220 nm; RT1 (min) 9.32)) to provide [(15R*,18S)-13,20-difluoro-15-{5-[2-(3-fluoroazetidin-1-yl)ethyl]-2-oxo-4-(trifluoromethyl)pyridin-1-yl}-3,4,21-trimethyl-16-oxo-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetic acid (4.3 mg, 14.5%) as a white solid. LC-MS: (ES+H, m/z) [M+H]⁺=728.30. ¹H NMR (400 MHz, CD₃OD) δ 7.46 (s, 1H), 7.30 (d, J=7.2 Hz, 1H), 7.21 (s, 1H), 7.15-6.99 (m, 4H), 6.93-6.81 (m, 2H), 6.62 (s, 1H), 5.72 (s, 1H), 5.27 (d, J=13.1 Hz, 1H), 5.05 (s, 1H), 4.96 (d, J=13.2 Hz, 1H), 3.91-3.44 (m, 2H), 3.27-3.02 (m, 2H), 2.91-2.38 (m, 6H), 2.29 (s, 3H), 2.19 (s, 3H), 1.87 (s, 3H). ¹⁹F NMR (377 MHz, CD₃OD) δ −64.91, −118.26, −128.56, −180.48.

### Example 52

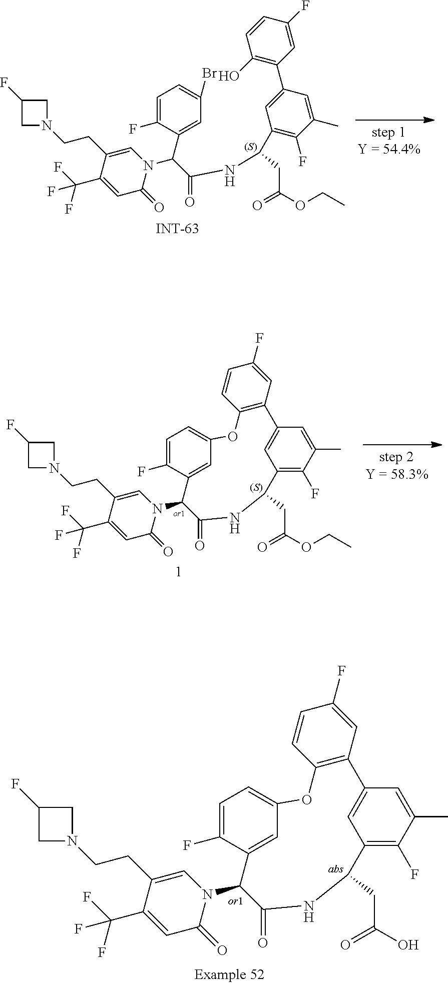

### Step 1: Preparation of ethyl 2-[(14S,17S)-4,12,19-trifluoro-14-{5-[2-(3-fluoroazetidin-1-yl)ethyl]-2-oxo-4-(trifluoromethyl)pyridin-1-yl}-20-methyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate

To a stirred solution of ethyl (3S)-3-[2-(5-bromo-2-fluorophenyl)-2-{5-[2-(3-fluoroazetidin-1-yl)ethyl]-2-oxo-4-(trifluoromethyl)pyridin-1-yl}acetamido]-3-{4,5′-difluoro-2′-hydroxy-5-methyl-[1,1′-biphenyl]-3-yl}propanoate (155 mg, 0.19 mmol, 1.00 equiv.) and CuI (18 mg, 0.10 mmol, 0.50 equiv.) in dioxane (2 mL) were added K₂CO₃ (53 mg, 0.38 mmol, 2.00 equiv.) and (1R,2R)—N1,N2-dimethylcyclohexane-1,2-diamine (27 mg, 0.19 mmol, 1.00 equiv.) in portions at room temperature under nitrogen atmosphere. The resulting mixture was stirred for 3 h at 100° C. under nitrogen atmosphere and then cooled to room temperature and concentrated. The residue was purified by reversed-phase flash chromatography (C18 gel; mobile phase, MeCN in water (0.1% NH₃·H₂O), 10% to 50% gradient in 30 min; detector, UV 220 nm) to provide ethyl 2-[(14S,17S)-4,12,19-trifluoro-14-{5-[2-(3-fluoroazetidin-1-yl)ethyl]-2-oxo-4-(trifluoromethyl)pyridin-1-yl}-20-methyl-15-oxo-8-oxa-16-azatetracyclo[16.3.1.1\hat{ }{9,13}.0\hat{ }{2,7}]tricosa-1(22),2(7),3,5,9,11,13(23), 18,20-nonaen-17-yl]acetate (76 mg, 54.4%) as a yellow solid. LC-MS: (ES+H, m/z) [M+H]⁺=732.0.

### Step 2: Preparation of [(14S,17S)-4,12,19-trifluoro-14-{5-[2-(3-fluoroazetidin-1-yl)ethyl]-2-oxo-4-(trifluoromethyl)pyridin-1-yl}-20-methyl-15-oxo-8-oxa-16-azatetracyclo[16.3.1.1\hat{ }{9,13}.0\hat{ }{2, 7}]tricosa-1(22),2(7),3,5,9,11,13(23),18,20-nonaen-17-yl]acetic acid

A solution of ethyl 2-[(14S,17S)-4,12,19-trifluoro-14-{5-[2-(3-fluoroazetidin-1-yl)ethyl]-2-oxo-4-(trifluoromethyl)pyridin-1-yl}-20-methyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (75 mg, 0.10 mmol, 1.00 equiv.) and LiOH·H₂O (13 mg, 0.31 mmol, 3.00 equiv.) in THF (1.5 mL) and H₂O (0.3 mL) was stirred for 3 h at room temperature under nitrogen atmosphere and then concentrated. The crude product was purified by Prep-HPLC (Column: XBridge Shield RP18 OBD Column 30*150 mm, 5 μm; Mobile Phase A: water (0.1% HCOOH), Mobile Phase B: MeCN; Flow rate: 60 mL/min; Gradient: 21% B to 42% B in 10 min; Wavelength: 254 nm/220 nm; RT1 (min) 9.63) to afford [(14S,17S)-4,12,19-trifluoro-14-{5-[2-(3-fluoroazetidin-1-yl)ethyl]-2-oxo-4-(trifluoromethyl)pyridin-1-yl}-20-methyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid (42.1 mg, 58.3%) as a white solid. LC-MS: (ES+H, m/z) [M+H]⁺=704.15. ¹H NMR (400 MHz, DMSO-d₆) δ 9.21 (d, J=7.9 Hz, 1H), 7.86 (dd, J=9.0, 5.0 Hz, 1H), 7.65 (dt, J=9.1, 3.6 Hz, 1H), 7.40-7.28 (m, 3H), 7.15 (dt, J=8.6, 2.9 Hz, 2H), 6.82 (s, 1H), 6.75 (s, 1H), 6.54 (dd, J=6.3, 2.3 Hz, 1H), 6.34 (dd, J=6.1, 3.0 Hz, 1H), 5.53 (td, J=8.3, 5.9 Hz, 1H), 4.86 (dt, J=57.8, 5.3 Hz, 1H), 2.87 (ddt, J=24.1, 8.6, 4.4 Hz, 2H), 2.62-2.53 (m, 2H), 2.51 (s, 2H), 2.45-2.34 (m, 1H), 2.35 (s, 3H), 2.30 (d, J=1.8 Hz, 3H). ¹⁹F NMR (377 MHz, DMSO-d₆) δ −62.60, −116.89, −121.65, −124.97, −177.16.

### Example 53

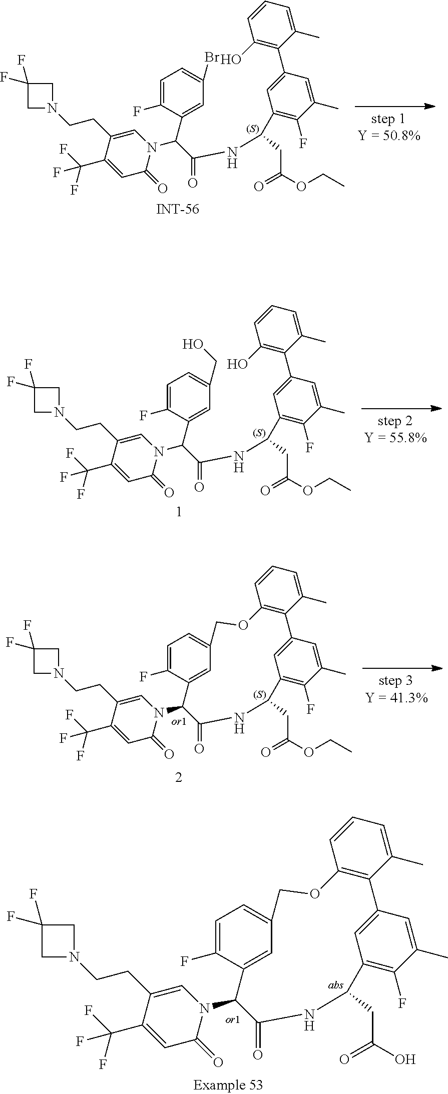

### Step 1: Preparation of ethyl (3S)-3-(2-{5-[2-(3,3-difluoroazetidin-1-yl)ethyl]-2-oxo-4-(trifluoromethyl)pyridin-1-yl}-2-[2-fluoro-5-(hydroxymethyl)phenyl]acetamido)-3-{4-fluoro-2′-hydroxy-5,6′-dimethyl-[1,1′-biphenyl]-3-yl}propanoate

To a stirred mixture of ethyl (3S)-3-[2-(5-bromo-2-fluorophenyl)-2-{5-[2-(3,3-difluoroazetidin-1-yl)ethyl]-2-oxo-4-(trifluoromethyl)pyridin-1-yl}acetamido]-3-{4-fluoro-2′-hydroxy-5,6′-dimethyl-[1,1′-biphenyl]-3-yl}propanoate (230 mg, 0.27 mmol, 1.00 equiv.) in 1,4-dioxane (5 mL) were added (tributylstannyl)methanol (268 mg, 0.83 mmol, 3.00 equiv.) and XPhos Pd G2 catalyst (21 mg, 0.02 mmol, 0.10 equiv.) at room temperature under nitrogen atmosphere. The resulting mixture was stirred overnight at 100° C. under nitrogen atmosphere and then cooled to room temperature. The resulting mixture was diluted with water (10 mL) and extracted with EtOAc (2×20 mL). The combined organic layers were washed with brine (2×10 mL), dried over anhydrous Na₂SO₄, filtered, and concentrated. The residue was purified by reversed-phase flash chromatography (C18 silica gel; mobile phase, MeCN in water (0.1% NH₃·H₂O), 40% to 60% gradient in 20 min; detector, UV 254 nm) to afford ethyl (3S)-3-(2-{5-[2-(3,3-difluoroazetidin-1-yl)ethyl]-2-oxo-4-(trifluoromethyl)pyridin-1-yl}-2-[2-fluoro-5-(hydroxymethyl)phenyl]acetamido)-3-{4-fluoro-2′-hydroxy-5,6′-dimethyl-[1,1′-biphenyl]-3-yl}propanoate (110 mg, 50.8%) as colorless oil. LC-MS: (ES+H, m/z) [M+H]⁺=778.3.

### Step 2: Preparation of ethyl 2-[(15S,18S)-15-{5-[2-(3,3-difluoroazetidin-1-yl)ethyl]-2-oxo-4-(trifluoromethyl)pyridin-1-yl}-13,20-difluoro-3,21-dimethyl-16-oxo-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetate

To a stirred mixture of ethyl (3S)-3-(2-{5-[2-(3,3-difluoroazetidin-1-yl)ethyl]-2-oxo-4-(trifluoromethyl)pyridin-1-yl}-2-[2-fluoro-5-(hydroxymethyl)phenyl]acetamido)-3-{4-fluoro-2′-hydroxy-5,6′-dimethyl-[1,1′-biphenyl]-3-yl}propanoate (100 mg, 0.12 mmol, 1.00 equiv.) in toluene (2 mL) was added 2-(tributyl-lambda5-phosphanylidene)acetonitrile (170 mg, 0.70 mmol, 5.00 equiv.) at room temperature under nitrogen atmosphere. The resulting mixture was stirred overnight at 100° C. under nitrogen atmosphere and then cooled to room temperature and concentrated. The residue was purified by reversed-phase flash chromatography (C18 gel; mobile phase, MeCN in water (0.1% NH₃·H₂O), 50% to 70% gradient in 20 min; detector, UV 254/220 nm) to afford ethyl 2-[(15S,18S)-15-{5-[2-(3,3-difluoroazetidin-1-yl)ethyl]-2-oxo-4-(trifluoromethyl)pyridin-1-yl}-13,20-difluoro-3,21-dimethyl-16-oxo-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetate (60 mg, 55.8%) as an off-white solid. LC-MS: (ES+H, m/z) [M+H]⁺=760.3.

### Step 3: Preparation of ethyl (3S)-3-[2-(5-bromo-2-fluorophenyl)-2-{5-[2-(3-fluoroazetidin-1-yl)ethyl]-2-oxo-4-(trifluoromethyl)pyridin-1-yl}acetamido]-3-{4-fluoro-2′-hydroxy-6′-methyl-[1,1′-biphenyl]-3-yl}propanoate

To a stirred mixture of ethyl 2-[(15S,18S)-15-{5-[2-(3,3-difluoroazetidin-1-yl)ethyl]-2-oxo-4-(trifluoromethyl)pyridin-1-yl}-13,20-difluoro-3,21-dimethyl-16-oxo-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetate (60 mg, 0.07 mmol, 1 equiv.) in THF (1 mL) was added LiOH·H₂O (6 mg, 0.15 mmol, 2.00 equiv.) in water (0.5 mL) at room temperature under nitrogen atmosphere. The resulting mixture was stirred for 1 h at room temperature under nitrogen atmosphere and then diluted with water (2 mL) and acidified to pH 5 with 1 M HCl (aq.). The resulting mixture was concentrated under vacuum. The crude product (50 mg) was purified by Prep-HPLC (Column: XBridge Shield RP18 OBD Column 30*150 mm, 5 μm; Mobile Phase A: water (0.1% HCO₂H), Mobile Phase B: MeCN; Flow rate: 60 mL/min; Gradient: 33% B to 52% B in 8 min; Wavelength: 254 nm/220 nm; RT1 (min) 10.17) to afford [(15S,18S)-15-{5-[2-(3,3-difluoroazetidin-1-yl)ethyl]-2-oxo-4-(trifluoromethyl)pyridin-1-yl}-13,20-difluoro-3,21-dimethyl-16-oxo-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetic acid (24 mg, 41.3%) as an off-white solid. LC-MS: (ES+H, m/z) [M+H]⁺=732.05. ¹H NMR (400 MHz, CD₃OD) δ 7.50-7.44 (m, 1H), 7.32 (dd, J=7.1, 2.2 Hz, 1H), 7.24-7.04 (m, 5H), 6.98-6.92 (m, 1H), 6.87-6.80 (m, 2H), 6.68-6.63 (m, 1H), 5.75 (q, J=8.5, 5.8 Hz, 1H), 5.31 (d, J=13.2 Hz, 1H), 5.00 (d, J=13.2 Hz, 1H), 3.40-3.32 (m, 4H), 2.77-2.51 (m, 4H), 2.45 (t, J=6.7 Hz, 2H), 2.32 (d, J=2.0 Hz, 3H), 1.97 (s, 3H). ¹⁹F NMR (377 MHz, CD₃OD) δ −64.97, −100.05, −118.36, −128.33.

### Example 54

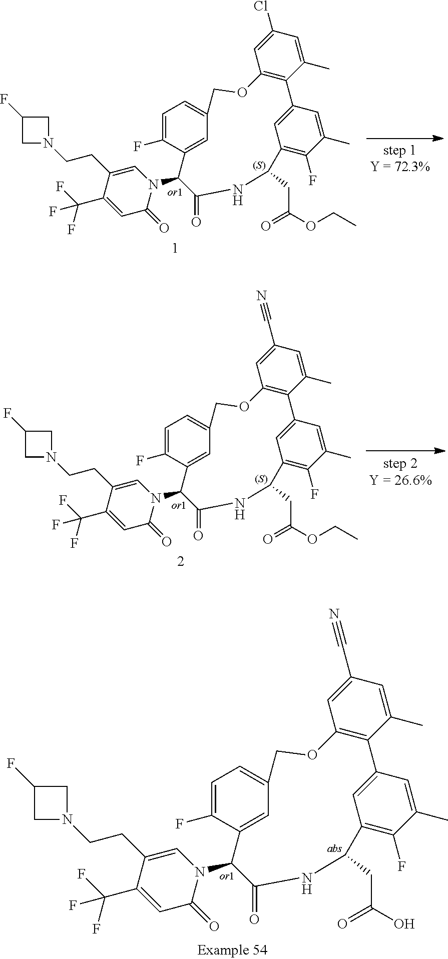

### Step 1: Preparation of ethyl 2-((6S,9S)-24-cyano-14,54-difluoro-6-(5-(2-(3-fluoroazetidin-1-yl)ethyl)-2-oxo-4-(trifluoromethyl)pyridin-1(2H)-yl)-15,26-dimethyl-7-oxo-3-oxa-8-aza-1,5(1,3),2(1,2)-tribenzenacyclononaphane-9-yl)acetate

A mixture of ethyl 2-[(15S,18S)-5-chloro-13,20-difluoro-15-{5-[2-(3-fluoroazetidin-1-yl)ethyl]-2-oxo-4-(trifluoromethyl)pyridin-1-yl}-3,21-dimethyl-16-oxo-8-oxa-17-azatetracyclo [17.3.1.1\hat{ }{10,14}.0\hat{ }{2,7}]tetracosa-1(23),2(7),3,5,10,12,14(24),19,21-nonaen-18-yl]acetate (70 mg, 0.09 mmol, 1.00 equiv.), Zn(CN)₂ (21 mg, 0.18 mmol, 2.00 equiv.), XPhos Pd G3 (7 mg, 0.009 mmol, 0.10 equiv.) and XPhos (8 mg, 0.01 mmol, 0.20 equiv.) in DMF (2 mL) was stirred for 2 h at 100° C. under nitrogen atmosphere. The mixture was purified by reversed-phase flash chromatography (C18 gel; mobile phase, MeCN in water (0.1% NH₃·H₂O), 30% to 60% gradient in 10 min; detector, UV 254 nm) to afford ethyl 2-((6S,9S)-24-cyano-14,54-difluoro-6-(5-(2-(3-fluoroazetidin-1-yl)ethyl)-2-oxo-4-(trifluoromethyl)pyridin-1(2H)-yl)-15,26-dimethyl-7-oxo-3-oxa-8-aza-1,5(1,3),2(1,2)-tribenzenacyclononaphane-9-yl)acetate (50 mg, 72.3%) as an off-white solid. LC-MS: (ES+H, m/z) [M+H]⁺=767.30.

### Step 2: Preparation of 2-((6S,9S)-24-cyano-14,54-difluoro-6-(5-(2-(3-fluoroazetidin-1-yl)ethyl)-2-oxo-4-(trifluoromethyl)pyridin-1(2H)-yl)-15,26-dimethyl-7-oxo-3-oxa-8-aza-1,5(1,3),2(1,2)-tribenzenacyclononaphane-9-yl)acetic acid

A mixture of ethyl 2-[(15S,18S)-5-cyano-13,20-difluoro-15-{5-[2-(3-fluoroazetidin-1-yl)ethyl]-2-oxo-4-(trifluoromethyl)pyridin-1-yl}-3,21-dimethyl-16-oxo-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetate (40 mg, 0.05 mmol, 1.00 equiv.) and LiOH·H₂O (4 mg, 0.10 mmol, 2.00 equiv.) in THF/H₂O=5/1 (0.6 mL) was stirred for 2 h at room temperature under nitrogen atmosphere. The mixture was acidified to pH 5 with 1 M HCl (aq.) and then concentrated. The crude product (40 mg) was purified by Prep-HPLC (Column: XSelect CSH Prep C18 OBD Column, 30*150 mm, 5 μm; Mobile Phase A: water (0.1% HCOOH), Mobile Phase B: MeCN; Flow rate: 60 mL/min; Gradient: 18% B to 38% B in 10 min; Wavelength: 254 nm/220 nm; RT1 (min) 9.85) to afford 2-((6S,9S)-24-cyano-14,54-difluoro-6-(5-(2-(3-fluoroazetidin-1-yl)ethyl)-2-oxo-4-(trifluoromethyl)pyridin-1(2H)-yl)-15,26-dimethyl-7-oxo-3-oxa-8-aza-1,5(1,3),2(1,2)-tribenzenacyclononaphane-9-yl)acetic acid (10.7 mg, 26.6%) as a white solid. LC-MS: (ES+H, m/z) [M+H]⁺=739.30. ¹H NMR (400 MHz, CD₃OD) δ 7.56 (s, 2H), 7.32 (s, 1H), 7.25-7.13 (m, 3H), 7.08 (s, 1H), 6.98 (d, J=6.6 Hz, 1H), 6.87 (s, 1H), 6.69 (d, J=6.3 Hz, 1H), 5.72 (t, J=7.1 Hz, 1H), 5.38 (d, J=13.3 Hz, 1H), 5.08 (d, J=13.2 Hz, 1H), 4.94-4.88 (m, 1H), 3.65-3.50 (m, 2H), 3.26-3.13 (m, 2H), 2.76-2.57 (m, 4H), 2.56-2.40 (m, 2H), 2.33 (s, 3H), 2.03 (s, 3H). ¹⁹F NMR (377 MHz, CD₃OD) δ −64.93, −118.08, −126.65, −180.52.

### Example 55

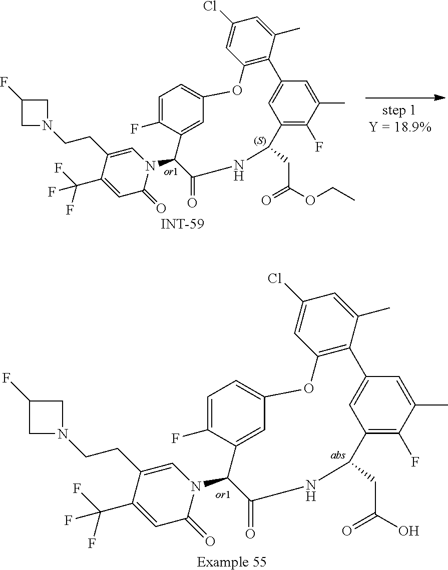

### Step 1: Preparation of [(14R*,17S)-5-chloro-12,19-difluoro-14-{5-[2-(3-fluoroazetidin-1-yl)ethyl]-2-oxo-4-(trifluoromethyl)pyridin-1-yl}-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid

To a stirred mixture of ethyl 2-[(14S,17S)-5-chloro-12,19-difluoro-14-{5-[2-(3-fluoroazetidin-1-yl)ethyl]-2-oxo-4-(trifluoromethyl)pyridin-1-yl}-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (50 mg, 0.07 mmol, 1.00 equiv.) in THF (1 mL) and H₂O (0.2 mL) was added LiOH·H₂O (8 mg, 0.20 mmol, 3.00 equiv.) at room temperature. The resulting mixture was stirred for 2 h at room temperature and then acidified to pH 3 with HCl (aq. 1 M) and concentrated. The crude product was purified by Prep-HPLC (Column: XBridge Prep OBD C18 Column, 30*150 mm, 10 μm; Mobile Phase A: water (10 mmol/L NH₄HCO₃+0.05% NH₃H₂O), Mobile Phase B: MeCN; Flow rate: 60 mL/min; Gradient: 35% B to 47% B in 8 min; Wavelength: 254 nm/220 nm; RT1 (min) 9.28) to afford [(14S,17S)-5-chloro-12,19-difluoro-14-{5-[2-(3-fluoroazetidin-1-yl)ethyl]-2-oxo-4-(trifluoromethyl)pyridin-1-yl}-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid (9.1 mg, 18.9%) as a white solid. LC-MS: (ES+H, m/z) [M+H]⁺=734.25. ¹H NMR (300 MHz, CD₃OD) δ 7.64-7.52 (m, 2H), 7.35 (s, 1H), 7.28-7.15 (m, 2H), 7.04-6.96 (m, 1H), 6.88 (d, J=2.1 Hz, 2H), 6.51 (dd, J=6.3, 2.1 Hz, 1H), 6.12 (dd, J=6.1, 3.0 Hz, 1H), 5.72 (t, J=7.5 Hz, 1H), 5.13-4.91 (m, 1H), 3.66-3.47 (m, 2H), 3.27-3.12 (m, 2H), 2.79-2.59 (m, 4H), 2.54-2.43 (m, 2H), 2.36 (d, J=1.9 Hz, 3H), 2.02 (s, 3H). ¹⁹F NMR (282 MHz, CD₃OD) δ −64.92, −122.50, −126.11, −180.31.

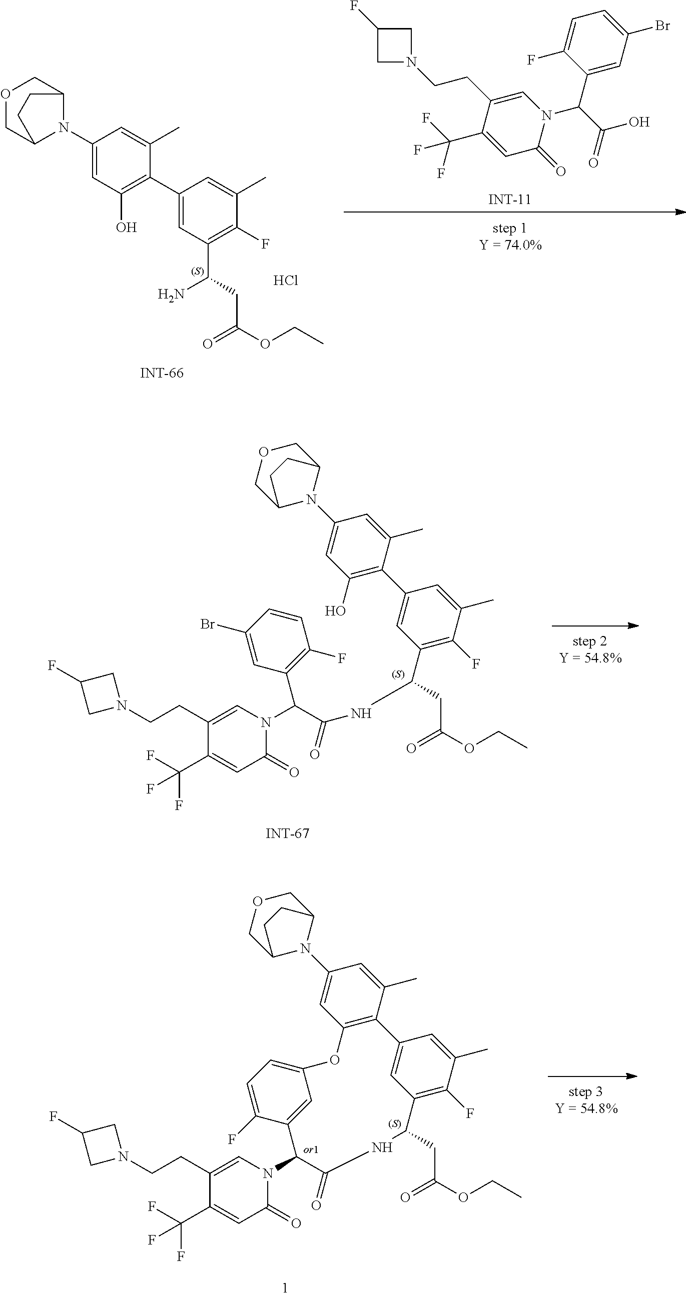

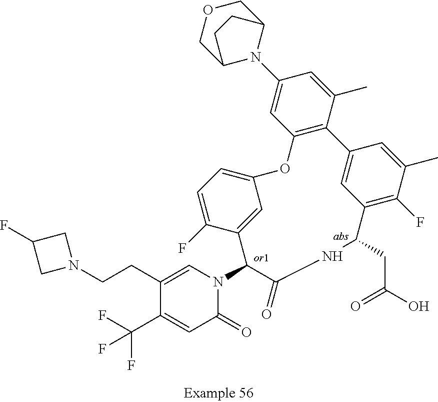 [SMILES: Cc1cc2cc(c1F)[C@H](CC(=O)O)NC(=O)[C@@H](n1cc(CCN3CC(F)C3)c(C(F)(F)F)cc1=O)c1cc(ccc1F)Oc1cc(N3C4CCC3COC4)cc(C)c1-2]

### Step 1: Preparation of ethyl (3S)-3-(4′-(3-oxa-8-azabicyclo[3.2.1]octan-8-yl)-4-fluoro-2′-hydroxy-5,6′-dimethyl-[1,1′-biphenyl]-3-yl)-3-(2-(5-bromo-2-fluorophenyl)-2-(5-(2-(3-fluoroazetidin-1-yl)ethyl)-2-oxo-4-(trifluoromethyl)pyridin-1(2H)-yl)acetamido)propanoate

A mixture of ethyl (3S)-3-(4′-(3-oxa-8-azabicyclo[3.2.1]octan-8-yl)-4-fluoro-2′-hydroxy-5,6′-dimethyl-[1,1′-biphenyl]-3-yl)-3-aminopropanoate hydrochloride (160 mg, 0.33 mmol, 1.03 equiv.) and NEt₃ (196 mg, 1.94 mmol, 6.00 equiv.) in MeCN (5 mL) was stirred for 30 min at 30° C. under nitrogen atmosphere and then concentrated to provide ethyl (3S)-3-(4′-(3-oxa-8-azabicyclo[3.2.1]octan-8-yl)-4-fluoro-2′-hydroxy-5,6′-dimethyl-[1,1′-biphenyl]-3-yl)-3-aminopropanoate. To a stirred mixture of 2-(5-bromo-2-fluorophenyl)-2-(5-(2-(3-fluoroazetidin-1-yl)ethyl)-2-oxo-4-(trifluoromethyl)pyridin-1(2H)-yl)acetic acid (160 mg, 0.32 mmol, 1.00 equiv.) and EDCI (93 mg, 0.49 mmol, 1.50 equiv.) in MeCN (5 mL) was added HOBT (57 mg, 0.42 mmol, 1.30 equiv.). The resulting mixture was stirred for 1 h at 30° C. under nitrogen atmosphere and then ethyl (3S)-3-(4′-(3-oxa-8-azabicyclo[3.2.1]octan-8-yl)-4-fluoro-2′-hydroxy-5,6′-dimethyl-[1,1′-biphenyl]-3-yl)-3-aminopropanoate in MeCN (2 mL) was added dropwise at room temperature. The resulting mixture was stirred overnight at 30° C. and then concentrated. The residue was purified by reversed-phase flash chromatography (C18 gel; mobile phase, MeCN in water (0.1% NH₃·H₂O), 40% to 70% gradient in 30 min; detector, UV 220 nm) to provide ethyl (3S)-3-(4′-(3-oxa-8-azabicyclo[3.2.1]octan-8-yl)-4-fluoro-2′-hydroxy-5,6′-dimethyl-[1,1′-biphenyl]-3-yl)-3-(2-(5-bromo-2-fluorophenyl)-2-(5-(2-(3-fluoroazetidin-1-yl)ethyl)-2-oxo-4-(trifluoromethyl)pyridin-1(2H)-yl)acetamido)propanoate (220 mg, mixture, 74.0%) as a yellow solid. LC-MS: (ES+H, m/z) [M+H]⁺=919.0.

### Step 2: Preparation of ethyl 2-[(14S,17S)-12,19-difluoro-14-{5-[2-(3-fluoroazetidin-1-yl)ethyl]-2-oxo-4-(trifluoromethyl)pyridin-1-yl}-3,20-dimethyl-5-{3-oxa-8-azabicyclo[3.2.1]octan-8-yl}-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate

To a stirred mixture of ethyl (3S)-3-(4′-(3-oxa-8-azabicyclo[3.2.1]octan-8-yl)-4-fluoro-2′-hydroxy-5,6′-dimethyl-[1,1′-biphenyl]-3-yl)-3-(2-(5-bromo-2-fluorophenyl)-2-(5-(2-(3-fluoroazetidin-1-yl)ethyl)-2-oxo-4-(trifluoromethyl)pyridin-1(2H)-yl)acetamido)propanoate (200 mg, 0.22 mmol, 1.00 equiv.) and (1R,2R)—N1,N2-dimethylcyclohexane-1,2-diamine (31 mg, 0.22 mmol, 1.00 equiv.) in dioxane (8 mL) were added CuI (21 mg, 0.11 mmol, 0.50 equiv.) and K₂CO₃ (60 mg, 0.43 mmol, 2.00 equiv.). The resulting mixture was stirred overnight at 100° C. under nitrogen atmosphere and then cooled to room temperature and diluted with EtOAc (100 mL). The combined organic layer was washed with water (3×50 mL), dried over anhydrous Na₂SO₄, filtered, and concentrated. The residue was purified by reversed-phase flash chromatography (C18 gel; mobile phase, MeCN in water (0.1% NH₃·H₂O), 40% to 70% gradient in 30 min; detector, UV 220 nm) to provide ethyl 2-[(14S,17S)-12,19-difluoro-14-{5-[2-(3-fluoroazetidin-1-yl)ethyl]-2-oxo-4-(trifluoromethyl)pyridin-1-yl}-3,20-dimethyl-5-{3-oxa-8-azabicyclo[3.2.1]octan-8-yl}-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (100 mg, 54.8%) as a yellow solid. LC-MS: (ES+H, m/z) [M+H]⁺=839.4. ¹H NMR (400 MHz, CD₃OD) δ 7.63-7.54 (m, 1H), 7.36 (s, 1H), 7.18 (t, J=9.0 Hz, 1H), 6.99 (d, J=12.0 Hz, 1H), 6.94 (d, J=2.4 Hz, 1H), 6.83 (d, J=18.9 Hz, 2H), 6.68 (d, J=2.3 Hz, 1H), 6.43 (dd, J=6.5, 2.2 Hz, 1H), 6.09 (dd, J=6.1, 2.9 Hz, 1H), 5.68 (t, J=7.8 Hz, 1H), 4.99-4.81 (m, 1H), 4.23 (s, 2H), 4.11 (qd, J=7.1, 4.5 Hz, 2H), 3.93 (t, J=10.8 Hz, 2H), 3.60-3.51 (m, 2H), 3.39 (tt, J=14.6, 7.1 Hz, 2H), 2.99 (ddd, J=23.3, 8.4, 4.7 Hz, 2H), 2.84 (dd, J=15.4, 8.4 Hz, 1H), 2.72 (dd, J=15.4, 7.4 Hz, 1H), 2.54-2.41 (m, 3H), 2.33 (d, J=2.0 Hz, 2H), 2.10-2.05 (m, 3H), 2.03 (s, 2H), 1.94 (s, 3H), 1.19 (t, J=7.1 Hz, 3H). ¹⁹F NMR (377 MHz, CD₃OD) δ −65.03, −123.38, −127.44, −180.02.

### Step 3: Preparation of [(14R*,17S)-12,19-difluoro-14-{5-[2-(3-fluoroazetidin-1-yl)ethyl]-2-oxo-4-(trifluoromethyl)pyridin-1-yl}-3,20-dimethyl-5-{3-oxa-8-azabicyclo[3.2.1]octan-8-yl}-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid

A mixture of ethyl 2-[(14S,17S)-12,19-difluoro-14-{5-[2-(3-fluoroazetidin-1-yl)ethyl]-2-oxo-4-(trifluoromethyl)pyridin-1-yl}-3,20-dimethyl-5-{3-oxa-8-azabicyclo[3.2.1]octan-8-yl}-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (100 mg, 0.12 mmol, 1.00 equiv.) and LiOH·H₂O (20 mg, 0.48 mmol, 4.00 equiv.) in THF (2 mL) and H₂O (0.5 mL) was stirred overnight at room temperature under nitrogen atmosphere. The mixture was then acidified to pH 5 with 3 M HCl (aq.) and concentrated. The crude product was purified by Prep-HPLC (Column: XBridge Shield RP18 OBD Column 30*150 mm, 5 μm; Mobile Phase A: water (0.1% HCOOH), Mobile Phase B: MeOH; Flow rate: 60 mL/min; Gradient: 39% B to 56% B in 8 min; Wavelength: 254 nm/220 nm; RT1 (min) 7.98) to afford [(14R*,17S)-12,19-difluoro-14-{5-[2-(3-fluoroazetidin-1-yl)ethyl]-2-oxo-4-(trifluoromethyl)pyridin-1-yl}-3,20-dimethyl-5-{3-oxa-8-azabicyclo[3.2.1]octan-8-yl}-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid (53.0 mg, 54.8%) as a white solid. LC-MS: (ES+H, m/z) [M+H]⁺=811.30. ¹H NMR (400 MHz, CD₃OD) δ 7.61-7.55 (m, 1H), 7.35 (s, 1H), 7.16 (t, J=9.0 Hz, 1H), 6.96 (dd, J=7.1, 2.1 Hz, 1H), 6.93 (d, J=2.4 Hz, 1H), 6.86 (s, 2H), 6.67 (d, J=2.3 Hz, 1H), 6.46 (dd, J=6.4, 2.2 Hz, 1H), 6.11 (dd, J=6.2, 2.9 Hz, 1H), 5.68 (t, J=7.4 Hz, 1H), 5.06-4.90 (m, 1H), 4.23 (s, 2H), 3.93 (t, J=10.7 Hz, 2H), 3.70-3.59 (m, 4H), 3.28-3.16 (m, 2H), 2.80-2.60 (m, 4H), 2.56-2.40 (m, 2H), 2.32 (s, 3H), 2.14-2.00 (m, 4H), 1.94 (s, 3H). ¹⁹F NMR (377 MHz, CD₃OD) δ −64.996, −123.292, −127.407, −180.332.

### Example 57

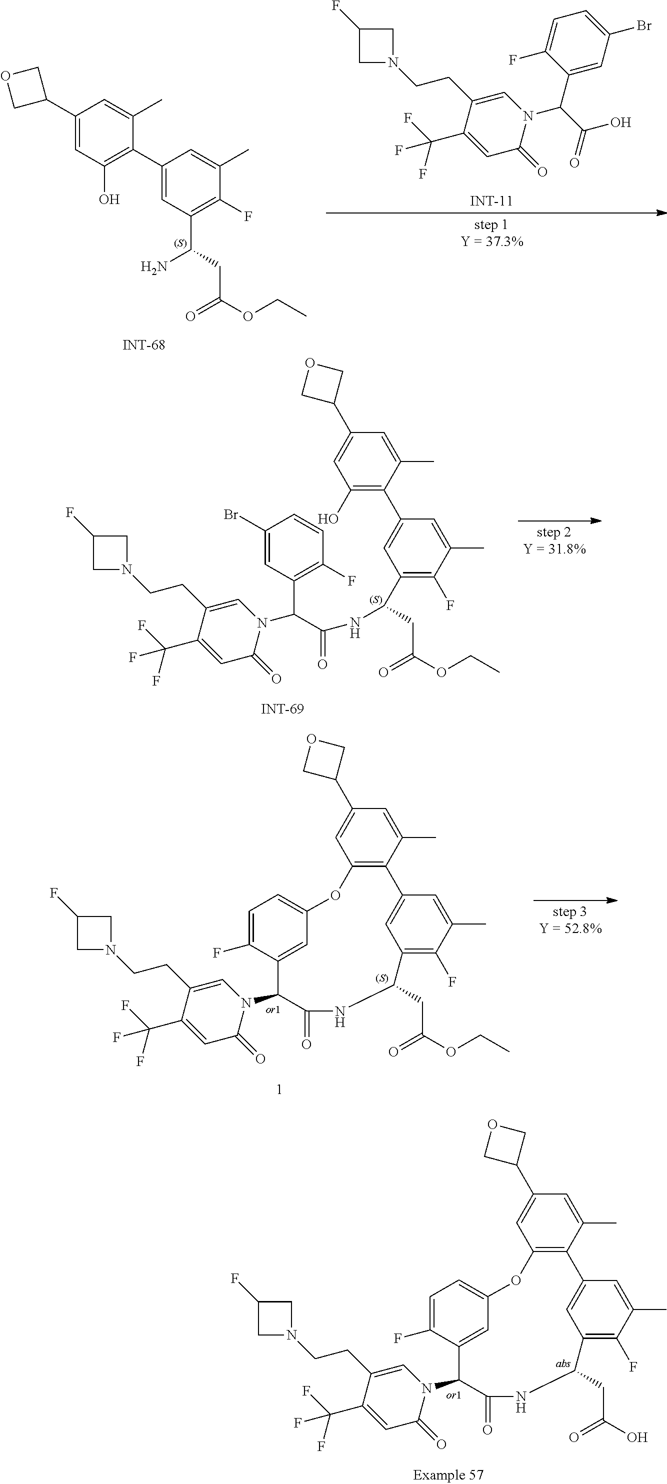

### Step 1: Preparation of ethyl (3S)-3-(2-(5-bromo-2-fluorophenyl)-2-(5-(2-(3-fluoroazetidin-1-yl)ethyl)-2-oxo-4-(trifluoromethyl)pyridin-1(2H)-yl)acetamido)-3-(4-fluoro-2′-hydroxy-5,6′-dimethyl-4′-(oxetan-3-yl)-[1,1′-biphenyl]-3-yl)propanoate

A mixture of ethyl (3S)-3-amino-3-[4-fluoro-2′-hydroxy-5,6′-dimethyl-4′-(oxetan-3-yl)-[1,1′-biphenyl]-3-yl]propanoate (60 mg, 0.15 mmol, 1.00 equiv.) and NEt₃ (94 mg, 0.93 mmol, 6.00 equiv.) in MeCN (2 mL) was stirred for 30 min at room temperature under nitrogen atmosphere, concentrated, and then redissolved in MeCN. In a separate vial, a mixture of (5-bromo-2-fluorophenyl)({5-[2-(3-fluoroazetidin-1-yl)ethyl]-2-oxo-4-(trifluoromethyl)pyridin-1-yl})acetic acid (76 mg, 0.15 mmol, 1.00 equiv.)(5-bromo-2-fluorophenyl)({5-[2-(3-fluoroazetidin-1-yl)ethyl]-2-oxo-4-(trifluoromethyl)pyridin-1-yl})acetic acid (76 mg, 0.15 mmol, 1.00 equiv.), HOBT (27 mg, 0.20 mmol, 1.30 equiv.), EDCI (44 mg, 0.23 mmol, 1.50 equiv.) in MeCN (2 mL) was stirred for 1 h at room temperature under nitrogen atmosphere and then ethyl (3S)-3-amino-3-[4-fluoro-2′-hydroxy-5,6′-dimethyl-4′-(oxetan-3-yl)-[1,1′-biphenyl]-3-yl]propanoate in MeCN was added. The resulting mixture was stirred for 2 h at 40° C. and then concentrated. The residue was purified by reversed-phase flash chromatography (C18 gel; mobile phase, MeCN in water (0.11% NH₃·H₂O), 10% to 50% gradient in 10 min; detector, UV 254 nm) to provide ethyl (3S)-3-(2-(5-bromo-2-fluorophenyl)-2-(5-(2-(3-fluoroazetidin-1-yl)ethyl)-2-oxo-4-(trifluoromethyl)pyridin-1(2H)-yl)acetamido)-3-(4-fluoro-2′-hydroxy-5,6′-dimethyl-4′-(oxetan-3-yl)-[1,1′-biphenyl]-3-yl)propanoate (50 mg, 37.3%) as a yellow solid. LC-MS: (ES+H, m/z) [M+H]⁺=864.0.

### Step 2: Preparation of ethyl 2-((5S,8S)-14,44-difluoro-5-(5-(2-(3-fluoroazetidin-1-yl)ethyl)-2-oxo-4-(trifluoromethyl)pyridin-1(2H)-yl)-15,26-dimethyl-24-(oxetan-3-yl)-6-oxo-3-oxa-7-aza-1,4(1,3),2(1,2)-tribenzenacyclooctaphane-8-yl)acetate

A mixture of ethyl (3S)-3-[2-(5-bromo-2-fluorophenyl)-2-{5-[2-(3-fluoroazetidin-1-yl)ethyl]-2-oxo-4-(trifluoromethyl)pyridin-1-yl}acetamido]-3-[4-fluoro-2′-hydroxy-5,6′-dimethyl-4′-(oxetan-3-yl)-[1,1′-biphenyl]-3-yl]propanoate (45 mg, 0.05 mmol, 1.00 equiv.), (1R,2R)—N1,N2-dimethylcyclohexane-1,2-diamine (7 mg, 0.05 mmol, 1.00 equiv.), CuI (4 mg, 0.02 mmol, 0.50 equiv.) and K₂CO₃ (14 mg, 0.10 mmol, 2.00 equiv.) in 1,4-dioxane (2 mL) was stirred for 1 h at 100° C. under nitrogen atmosphere. The resulting mixture was then cooled to room temperature and concentrated. The residue was purified by reversed-phase flash chromatography (C18 gel; mobile phase, MeCN in water (0.1% NH₃·H₂O), 40% to 60% gradient in 10 min; detector, UV 254 nm) to provide ethyl 2-((5S,8S)-14,44-difluoro-5-(5-(2-(3-fluoroazetidin-1-yl)ethyl)-2-oxo-4-(trifluoromethyl)pyridin-1(2H)-yl)-15,26-dimethyl-24-(oxetan-3-yl)-6-oxo-3-oxa-7-aza-1,4(1,3),2(1,2)-tribenzenacyclooctaphane-8-yl)acetate (13 mg, 31.8%) as an off-white solid. LC-MS: (ES+H, m/z) [M+H]⁺=784.4.

### Step 3: Preparation of 2-((5S,8S)-14,44-difluoro-5-(5-(2-(3-fluoroazetidin-1-yl)ethyl)-2-oxo-4-(trifluoromethyl)pyridin-1(2H)-yl)-15,26-dimethyl-24-(oxetan-3-yl)-6-oxo-3-oxa-7-aza-1,4(1,3),2(1,2)-tribenzenacyclooctaphane-8-yl)acetic acid

A mixture of ethyl 2-[(14S,17S)-12,19-difluoro-14-{5-[2-(3-fluoroazetidin-1-yl)ethyl]-2-oxo-4-(trifluoromethyl)pyridin-1-yl}-3,20-dimethyl-5-(oxetan-3-yl)-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (13 mg, 0.017 mmol, 1.00 equiv.) and LiOH·H₂O (1 mg, 0.03 mmol, 2.00 equiv.) in THF/H₂O=5/1 (1.2 mL) was stirred for 1 h at room temperature under nitrogen atmosphere. The resulting mixture was then concentrated and the crude product was purified by Prep-HPLC (Column: Xselect CSH C18 OBD Column 30*150 mm, 5 μm; Mobile Phase A: water (0.1% HCOOH), Mobile Phase B: MeCN; Flow rate: 60 mL/min; Gradient: 16% B to 35% B in 8 min; Wavelength: 254 nm/220 nm; RT1 (min) 10.08) to afford 2-((5S,8S)-14,44-difluoro-5-(5-(2-(3-fluoroazetidin-1-yl)ethyl)-2-oxo-4-(trifluoromethyl)pyridin-1(2H)-yl)-15,26-dimethyl-24-(oxetan-3-yl)-6-oxo-3-oxa-7-aza-1,4(1,3),2(1,2)-tribenzenacyclooctaphane-8-yl)acetic acid (6.7 mg, 52.8%) as a white solid. LC-MS: (ES+H, m/z) [M+H]⁺=756.25. ¹H NMR (300 MHz, CD₃OD) δ 7.59 (dt, 1H), 7.51 (s, 1H), 7.35 (s, 1H), 7.24 (s, 1H), 7.17 (t, 1H), 6.97 (d, 1H), 6.87 (d, 2H), 6.47 (d, 1H), 6.11 (dd, 1H), 5.69 (m, 1H), 5.19-5.07 (m, 2H), 4.95-4.87 (m, 2H), 4.84-4.78 (m, 2H) 4.40-4.30 (m, 1H), 3.55-3.74 (s, 2H), 3.25 (d, 1H), 3.19-3.26 (m, 1H), 2.85-2.60 (m, 4H), 2.50 (s, 2H), 2.34 (d, 3H), 2.02 (d, 3H). ¹⁹F NMR (282 MHz, CD₃OD) δ −64.64, −80.14, −123.05, −126.65, −180.56.

### Example 58

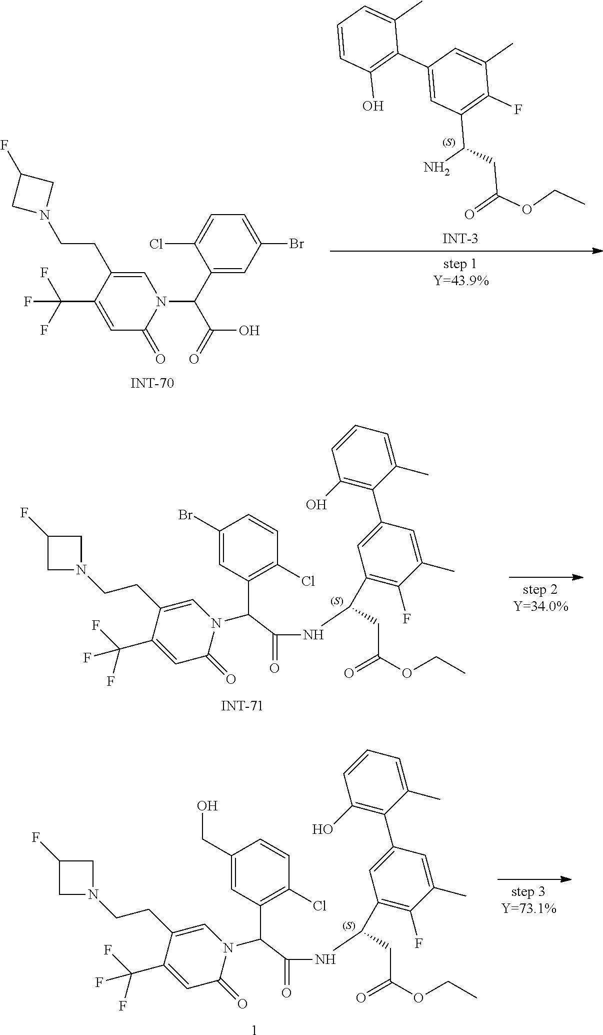

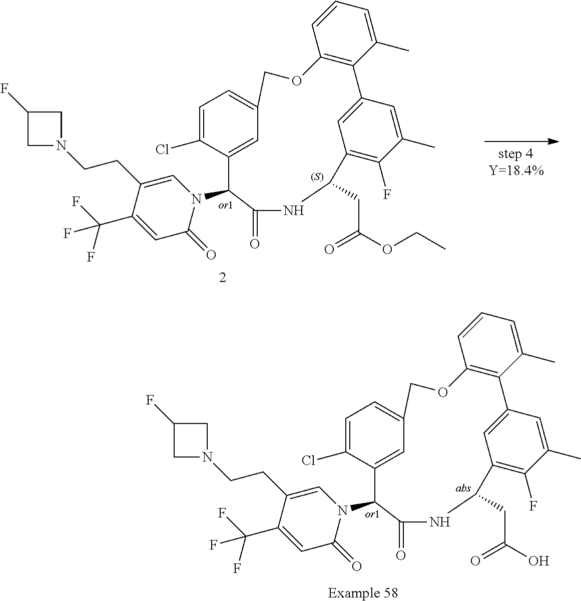

### Step 1: Preparation of ethyl (3S)-3-[2-(5-bromo-2-chlorophenyl)-2-{5-[2-(3-fluoroazetidin-1-yl)ethyl]-2-oxo-4-(trifluoromethyl)pyridin-1-yl}acetamido]-3-{4-fluoro-2′-hydroxy-5,6′-dimethyl-[1,1′-biphenyl]-3-yl}propanoate

A solution of ethyl (3S)-3-amino-3-{4-fluoro-2′-hydroxy-5,6′-dimethyl-[1,1′-biphenyl]-3-yl}propanoate (648 mg, 1.95 mmol, 1.54 equiv.) and Et₃N (642 mg, 6.35 mmol, 5.00 equiv.) in MeCN (15 mL) was stirred for 30 min at 30° C. under nitrogen atmosphere, concentrated and redissolved in MeCN. In a separate vial, a stirred solution of (5-bromo-2-chlorophenyl)({5-[2-(3-fluoroazetidin-1-yl)ethyl]-2-oxo-4-(trifluoromethyl)pyridin-1-yl})acetic acid (650 mg, 1.27 mmol, 1.00 equiv.) in MeCN (10 mL) was treated with HOBT (223 mg, 1.65 mmol, 1.30 equiv.) and EDCI (365 mg, 1.90 mmol, 1.50 equiv.) at room temperature under nitrogen atmosphere. The reaction mixture stirred for 1 h at 30° C. and then the solution of ethyl (S)-3-amino-3-(4′-cyclopropyl-4-fluoro-2′-hydroxy-6′-methyl-5-(trifluoromethyl)-[1,1′-biphenyl]-3-yl)propanoate in MeCN (5 mL) was add dropwise over 2 min at 30° C. The resulting mixture was stirred overnight at 30° C. and then diluted with EtOAc (100 mL), washed with water (3×80 mL), dried over anhydrous Na₂SO₄, filtered, and concentrated. The residue was purified by reversed-phase flash chromatography (C18 gel; mobile phase, MeCN in water (0.1% NH₃·H₂O), 0% to 70% gradient in 50 min; detector, UV 254 nm) to provide ethyl (3S)-3-[2-(5-bromo-2-chlorophenyl)-2-{5-[2-(3-fluoroazetidin-1-yl)ethyl]-2-oxo-4-(trifluoromethyl)pyridin-1-yl}acetamido]-3-{4-fluoro-2′-hydroxy-5,6′-dimethyl-[1,1′-biphenyl]-3-yl}propanoate (500 mg, 42.9%, mixture) as a colorless oil. LC-MS: (ES+H, m/z) [M+H]⁺=824.1.

### Step 2: Preparation of ethyl (3S)-3-{2-[2-chloro-5-(hydroxymethyl)phenyl]-2-{5-[2-(3-fluoroazetidin-1-yl)ethyl]-2-oxo-4-(trifluoromethyl)pyridin-1-yl}acetamido}-3-{4-fluoro-2′-hydroxy-5,6′-dimethyl-[1,1′-biphenyl]-3-yl}propanoate

To a stirred solution of ethyl (3S)-3-[2-(5-bromo-2-chlorophenyl)-2-{5-[2-(3-fluoroazetidin-1-yl)ethyl]-2-oxo-4-(trifluoromethyl)pyridin-1-yl}acetamido]-3-{4-fluoro-2′-hydroxy-5,6′-dimethyl-[1,1′-biphenyl]-3-yl}propanoate (250 mg, 0.30 mmol, 1.00 equiv.) and 2nd generation XPhos precatalyst (24 mg, 0.03 mmol, 0.10 equiv.) in 1,4-dioxane (6 mL) was added (tributylstannyl)methanol (292 mg, 0.91 mmol, 3.00 equiv.) dropwise. The reaction was stirred overnight at 80° C. and then concentrated. The residue was purified by reversed-phase flash chromatography (C18 gel; mobile phase, MeCN in water (0.1% NH₃·H₂O), 0% to 50% gradient in 20 min; detector, UV 220 nm) to provide ethyl (3S)-3-{2-[2-chloro-5-(hydroxymethyl)phenyl]-2-{5-[2-(3-fluoroazetidin-1-yl)ethyl]-2-oxo-4-(trifluoromethyl)pyridin-1-yl}acetamido}-3-{4-fluoro-2′-hydroxy-5,6′-dimethyl-[1,1′-biphenyl]-3-yl}propanoate (80 mg, 34.0%) as a yellow oil. LC-MS: (ES+H, m/z) [M+H]⁺=776.40.

### Step 3: Preparation of ethyl 2-[(15S,18S)-13-chloro-20-fluoro-15-{5-[2-(3-fluoroazetidin-1-yl)ethyl]-2-oxo-4-(trifluoromethyl)pyridin-1-yl}-3,21-dimethyl-16-oxo-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetate

A solution of ethyl (3S)-3-{2-[2-chloro-5-(hydroxymethyl)phenyl]-2-{5-[2-(3-fluoroazetidin-1-yl)ethyl]-2-oxo-4-(trifluoromethyl)pyridin-1-yl}acetamido}-3-{4-fluoro-2′-hydroxy-5,6′-dimethyl-[1,1′-biphenyl]-3-yl}propanoate (70 mg, 0.09 mmol, 1.00 equiv.) and 2-(tributyl-lambda5-phosphanylidene)acetonitrile (108 mg, 0.45 mmol, 5.00 equiv.) in toluene (3 mL) was stirred overnight at 110° C. under nitrogen atmosphere. The resulting mixture was concentrated under reduced pressure and the residue was purified by reversed-phase flash chromatography (C18 gel; mobile phase, MeCN in water (0.1% NH₃·H₂O), 0% to 50% gradient in 10 min; detector, UV 220 nm) to provide ethyl 2-[(15S,18S)-13-chloro-20-fluoro-15-{5-[2-(3-fluoroazetidin-1-yl)ethyl]-2-oxo-4-(trifluoromethyl)pyridin-1-yl}-3,21-dimethyl-16-oxo-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetate (50 mg, 73.1%) as a white solid. LC-MS: (ES+H, m/z) [M+H]⁺=758.10. ¹H NMR (400 MHz, DMSO-d₆) δ 9.47 (d, J=7.6 Hz, 1H), 7.57 (d, J=8.4 Hz, 1H), 7.52 (d, J=8.0 Hz, 1H), 7.30 (d, J=8.3 Hz, 1H), 7.22-7.16 (m, 2H), 7.02 (d, J=7.1 Hz, 1H), 6.97 (s, 1H), 6.82 (d, J=11.2 Hz, 3H), 6.49 (d, J=6.8 Hz, 1H), 5.59 (s, 1H), 5.41 (d, J=13.3 Hz, 1H), 5.04 (d, J=13.3 Hz, 1H), 4.95-4.72 (m, 1H), 4.13-3.94 (m, 2H), 2.88-2.73 (m, 3H), 2.26 (d, J=13.7 Hz, 8H), 1.92 (s, 3H), 1.14 (t, J=7.1 Hz, 3H).

### Step 4: Preparation of [(15S,18S)-13-chloro-20-fluoro-15-{5-[2-(3-fluoroazetidin-1-yl)ethyl]-2-oxo-4-(trifluoromethyl)pyridin-1-yl}-3,21-dimethyl-16-oxo-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetic acid

A solution of ethyl 2-[(15S,18S)-13-chloro-20-fluoro-15-{5-[2-(3-fluoroazetidin-1-yl)ethyl]-2-oxo-4-(trifluoromethyl)pyridin-1-yl}-3,21-dimethyl-16-oxo-8-oxa-17-azatetracyclo [17.3.1.1\hat{ }{10,14}.0\hat{ }{2,7}]tetracosa-1(23),2(7),3,5,10,12,14(24),19,21-nonaen-18-yl]acetate (40 mg, 0.05 mmol, 1.00 equiv.) and LiOH·H₂O (6 mg, 0.16 mmol, 3.00 equiv.) in THF:H₂O=2 mL:0.5 mL was stirred for 2 h at room temperature under nitrogen atmosphere. The mixture was then acidified to pH 4 with 1 M HCl (aq.) and concentrated. The crude product was purified by Prep-HPLC (Column: Xbridge Phenyl OBD Column, 19*150 mm, 5 μm; Mobile Phase A: water (10 mmol/L NH₄HCO₃, Mobile Phase B: MeCN; Flow rate: 60 mL/min; Gradient: 25% B to 40% B in 10 min; Wavelength: 254 nm/220 nm; RT1 (min) 10.62) to afford [(15S,18S)-13-chloro-20-fluoro-15-{5-[2-(3-fluoroazetidin-1-yl)ethyl]-2-oxo-4-(trifluoromethyl)pyridin-1-yl}-3,21-dimethyl-16-oxo-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetic acid (7 mg, 18.4%) as a white solid. LC-MS: (ES+H, m/z) [M+H]⁺=730.30. ¹H NMR (400 MHz, DMSO-d₆) δ 9.57 (s, 1H), 7.56 (dd, J=8.1, 2.0 Hz, 1H), 7.51 (d, J=8.0 Hz, 1H), 7.30 (d, J=8.4 Hz, 1H), 7.23-7.20 (m, 1H), 7.18 (d, J=8.1 Hz, 1H), 7.00 (d, J=4.6 Hz, 2H), 6.86 (s, 1H), 6.84-6.78 (m, 2H), 6.51-6.41 (m, 1H), 5.41 (d, J=13.2 Hz, 1H), 5.04 (d, J=13.3 Hz, 1H), 4.90 (p, J=5.5 Hz, 1H), 4.75 (t, J=5.3 Hz, 1H), 3.18 (dt, J=14.7, 6.9 Hz, 3H), 2.90-2.74 (m, 2H), 2.58 (d, J=15.5 Hz, 1H), 2.39 (d, J=7.2 Hz, 2H), 2.32-2.22 (m, 6H), 1.90 (s, 3H). ¹⁹F NMR (377 MHz, DMSO) δ −62.50, −127.50, −176.90.

### Example 59

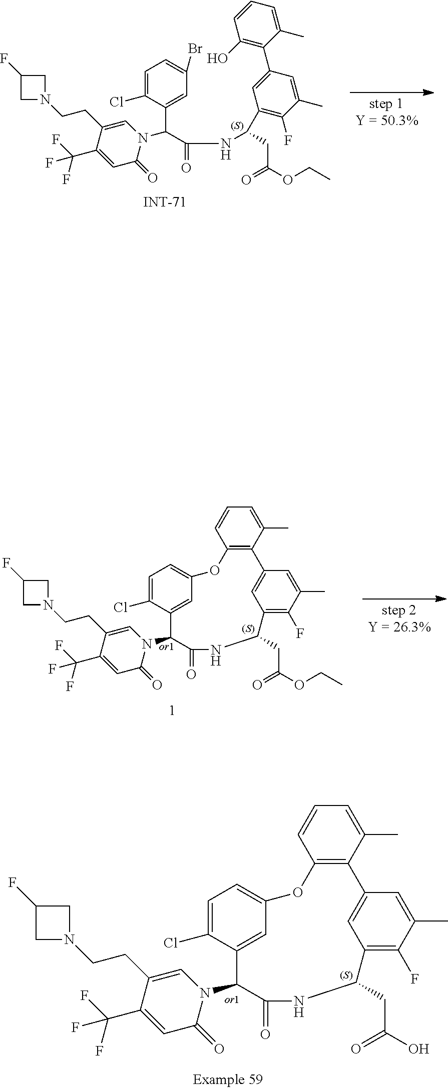

### Step 1: Preparation of ethyl 2-[(14R*,17S)-12-chloro-19-fluoro-14-{5-[2-(3-fluoroazetidin-1-yl)ethyl]-2-oxo-4-(trifluoromethyl)pyridin-1-yl}-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate

To a stirred solution of ethyl (3S)-3-[2-(5-bromo-2-chlorophenyl)-2-{5-[2-(3-fluoroazetidin-1-yl)ethyl]-2-oxo-4-(trifluoromethyl)pyridin-1-yl}acetamido]-3-{4-fluoro-2′-hydroxy-5,6′-dimethyl-[1,1′-biphenyl]-3-yl}propanoate (130 mg, 0.16 mmol, 1.00 equiv.) and (1R,2R)—N1,N2-dimethylcyclohexane-1,2-diamine (22 mg, 0.16 mmol, 1.00 equiv.) in 1,4-dioxane (6 mL) were added CuI (15 mg, 0.08 mmol, 0.50 equiv.) and K₂CO₃ (65 mg, 0.47 mmol, 3.00 equiv.) dropwise at room temperature under nitrogen atmosphere. The resulting mixture was stirred overnight at 100° C. and then cooled to room temperature and concentrated. The residue was purified by reversed-phase flash chromatography (C18 gel; mobile phase, MeCN in water (0.1% NH₃·H₂O), 0% to 80% gradient in 30 min; detector, UV 254 nm) to afford ethyl 2-[(14R*,17S)-12-chloro-19-fluoro-14-{5-[2-(3-fluoroazetidin-1-yl)ethyl]-2-oxo-4-(trifluoromethyl)pyridin-1-yl}-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (59 mg, 50.3%) as a brown yellow solid. LC-MS: (ES+H, m/z) [M+H]⁺=744.0. ¹H NMR (400 MHz, DMSO-d₆) δ 7.72-7.59 (m, 2H), 7.55 (d, J=8.9 Hz, 1H), 7.38 (t, J=7.9 Hz, 1H), 7.21 (d, J=7.6 Hz, 1H), 7.12 (s, 1H), 7.04 (d, J=6.1 Hz, 1H), 6.81 (s, 1H), 6.57 (s, 1H), 6.53-6.47 (m, 1H), 6.10 (d, J=2.8 Hz, 1H), 5.56-5.46 (m, 1H), 4.16-3.97 (m, 2H), 3.31-3.14 (m, 3H), 2.87 (dd, J=7.7, 4.4 Hz, 1H), 2.82 (t, J=7.8 Hz, 2H), 2.73 (dd, J=15.7, 6.6 Hz, 1H), 2.41 (s, 2H), 2.34-2.22 (m, 6H), 1.97 (s, 3H), 1.13 (t, J=7.1 Hz, 3H).

### Step 2: Preparation of [(14R*,17S)-12-chloro-19-fluoro-14-{5-[2-(3-fluoroazetidin-1-yl)ethyl]-2-oxo-4-(trifluoromethyl)pyridin-1-yl}-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid

To a stirred solution of ethyl 2-[(14R*,17S)-12-chloro-19-fluoro-14-{5-[2-(3-fluoroazetidin-1-yl)ethyl]-2-oxo-4-(trifluoromethyl)pyridin-1-yl}-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (54 mg, 0.07 mmol, 1.00 equiv.) in THF (2 mL) and H₂O (0.5 mL) was added LiOH·H₂O (12 mg, 0.29 mmol, 4.00 equiv.) dropwise at room temperature under nitrogen atmosphere. The resulting mixture was stirred for 6 h at room temperature and then concentrated. The crude product was purified by Prep-HPLC (Column: Xbridge Phenyl C18 Column, 30*150 mm, 5 μm; Mobile Phase A: water (10 mmol/L NH₄HCO₃), Mobile Phase B: CH₃CN; Flow rate: 60 mL/min; Gradient: 25% B to 40% B in 15 min; Wavelength: 254 nm/220 nm; RT1 (min) 9.2) to afford [(14R*,17S)-12-chloro-19-fluoro-14-{5-[2-(3-fluoroazetidin-1-yl)ethyl]-2-oxo-4-(trifluoromethyl)pyridin-1-yl}-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid (14.4 mg, 26.3%) as a white solid. LC-MS: (ES+H, m/z) [M+H]⁺=716.05. ¹H NMR (400 MHz, CD₃OD) δ 7.58 (dd, J=8.9, 2.8 Hz, 1H), 7.51-7.43 (m, 2H), 7.35 (t, J=7.9 Hz, 1H), 7.26 (s, 1H), 7.17 (d, J=7.6 Hz, 1H), 6.97 (d, J=6.2 Hz, 1H), 6.88 (s, 1H), 6.82 (s, 1H), 6.55-6.48 (m, 1H), 6.24 (d, J=2.8 Hz, 1H), 5.72 (t, J=7.5 Hz, 1H), 5.04 (t, J=5.1 Hz, 1H), 3.56 (dt, J=16.7, 8.7 Hz, 2H), 3.30-3.16 (m, 2H), 2.81-2.56 (m, 4H), 2.47 (h, J=8.1 Hz, 2H), 2.34 (d, J=1.9 Hz, 3H), 2.02 (s, 3H). ¹⁹F NMR (377 MHz, CD₃OD) δ −64.97, −126.84, −180.46.

### Example 60

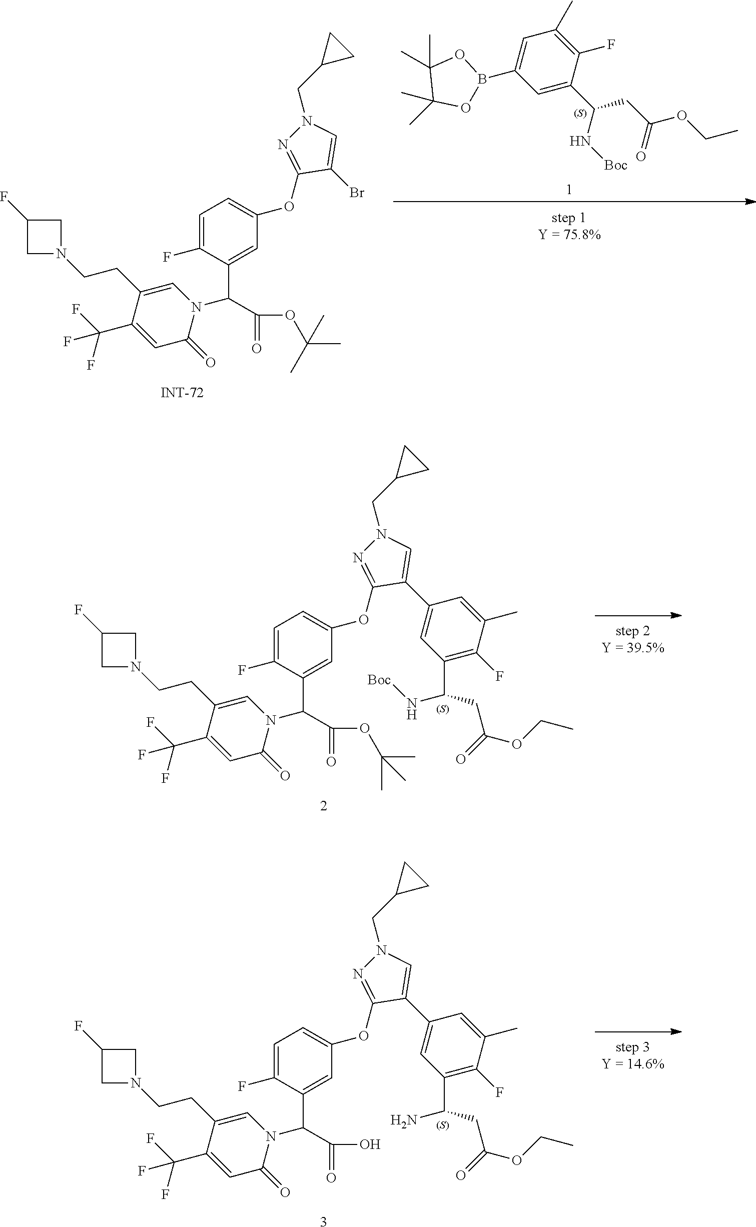

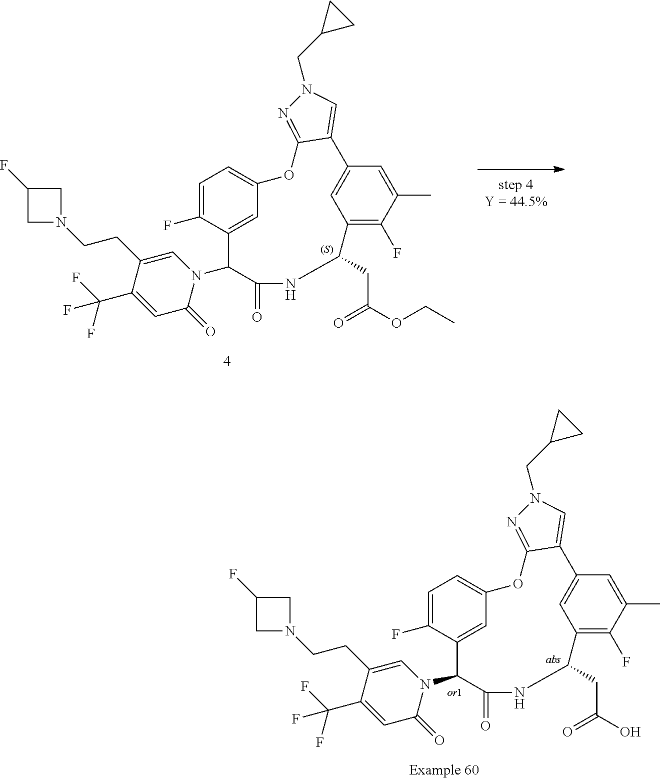

### Step 1: Preparation of ethyl (3S)-3-[5-(3-{3-[2-(tert-butoxy)-1-{5-[2-(3-fluoroazetidin-1-yl)ethyl]-2-oxo-4-(trifluoromethyl)pyridin-1-yl}-2-oxoethyl]-4-fluorophenoxy}-1-(cyclopropylmethyl)pyrazol-4-yl)-2-fluoro-3-methylphenyl]-3-[(tert-butoxycarbonyl)amino]propanoate

To a stirred solution of tert-butyl 2-(5-{[4-bromo-1-(cyclopropylmethyl)pyrazol-3-yl]oxy}-2-fluorophenyl)-2-{5-[2-(3-fluoroazetidin-1-yl)ethyl]-2-oxo-4-(trifluoromethyl)pyridin-1-yl}acetate (700 mg, 1.01 mmol, 1.00 equiv.) and ethyl (3S)-3-[(tert-butoxycarbonyl)amino]-3-[2-fluoro-3-methyl-5-(4,4,5,5-tetramethyl-1,3,2-dioxaborolan-2-yl)phenyl]propanoate (551 mg, 1.22 mmol, 1.20 equiv.) in dioxane (15 mL) and H₂O (1.5 mL) were added Pd(OAc)₂ (45 mg, 0.20 mmol, 0.20 equiv.), XPhos (48 mg, 0.10 mmol, 0.10 equiv.) and K₂CO₃ (422 mg, 3.05 mmol, 3.00 equiv.) in portions at room temperature. The resulting mixture was stirred overnight at 100° C. under nitrogen atmosphere and then cooled to room temperature. The resulting mixture was filtered, and the filter cake was washed with CH₂Cl₂/MeOH (5:1, 3×50 mL). The filtrate was concentrated under reduced pressure and the residue was purified by reversed-phase flash chromatography (C18 gel; mobile phase, MeCN in water (0.1% NH₃·H₂O), 0% to 100% gradient in 50 min; detector, UV 254 nm) to provide ethyl (3S)-3-[5-(3-{3-[2-(tert-butoxy)-1-{5-[2-(3-fluoroazetidin-1-yl)ethyl]-2-oxo-4-(trifluoromethyl)pyridin-1-yl}-2-oxoethyl]-4-fluorophenoxy}-1-(cyclopropylmethyl)pyrazol-4-yl)-2-fluoro-3-methylphenyl]-3-[(tert-butoxycarbonyl)amino]propanoate (800 mg, 75.8%) as a colorless oil. LC-MS: (ES+H, m/z) [M+H]⁺=932.4. ¹H NMR (400 MHz, CD₃OD) δ7.98 (s, 1H), 7.40 (d, J=3.3 Hz, 1H), 7.37-7.33 (m, 2H), 7.29-7.24 (m, 1H), 7.16 (dd, J=11.5, 7.7 Hz, 1H), 6.86 (s, 1H), 6.55 (d, J=5.7 Hz, 1H), 5.27 (s, 1H), 5.08-4.88 (m, 1H), 4.10-4.03 (m, 2H), 3.91 (d, J=7.1 Hz, 2H), 3.54-3.39 (m, 2H), 3.13-2.97 (m, 2H), 2.76-2.66 (m, 2H), 2.59-2.40 (m, 4H), 2.23 (d, J=2.1 Hz, 3H), 1.39 (s, 18H), 1.30-1.25 (m, 1H), 1.19-1.14 (m, 3H), 0.66-0.61 (m, 2H), 0.43-0.39 (m, 2H).

### Step 2: Preparation of {5-[(4-{3-[(1S)-1-amino-3-ethoxy-3-oxopropyl]-4-fluoro-5-methylphenyl}-1-(cyclopropylmethyl)pyrazol-3-yl)oxy]-2-fluorophenyl}({5-[2-(3-fluoroazetidin-1-yl)ethyl]-2-oxo-4-(trifluoromethyl)pyridin-1-yl})acetic acid

To a stirred solution of ethyl (3S)-3-[5-(3-{3-[2-(tert-butoxy)-1-{5-[2-(3-fluoroazetidin-1-yl)ethyl]-2-oxo-4-(trifluoromethyl)pyridin-1-yl}-2-oxoethyl]-4-fluorophenoxy}-1-(cyclopropylmethyl)pyrazol-4-yl)-2-fluoro-3-methylphenyl]-3-[(tert-butoxycarbonyl)amino]propanoate (800 mg, 0.85 mmol, 1.00 equiv.) in DCM (10 mL) was added TFA (10 mL) dropwise at room temperature. The resulting mixture was stirred for 3 h at room temperature and then concentrated. The residue was purified by reversed-phase flash chromatography (C18 gel; mobile phase, MeCN in water, 0% to 100% gradient in 50 min; detector, UV 254 nm) to provide {5-[(4-{3-[(1S)-1-amino-3-ethoxy-3-oxopropyl]-4-fluoro-5-methylphenyl}-1-(cyclopropylmethyl)pyrazol-3-yl)oxy]-2-fluorophenyl}({5-[2-(3-fluoroazetidin-1-yl)ethyl]-2-oxo-4-(trifluoromethyl)pyridin-1-yl})acetic acid (310 mg, 39.5%) as a colorless oil. LC-MS: (ES+H, m/z) [M+H]⁺=776.3

### Step 3: Preparation of ethyl 2-((8S)-21-(cyclopropylmethyl)-14,44-difluoro-5-(5-(2-(3-fluoroazetidin-1-yl)ethyl)-2-oxo-4-(trifluoromethyl)pyridin-1(2H)-yl)-15-methyl-6-oxo-21H-3-oxa-7-aza-2(4,3)-pyrazola-1,4(1,3)-dibenzenacyclooctaphane-8-yl)acetate

A mixture of {5-[(4-{3-[(1S)-1-amino-3-ethoxy-3-oxopropyl]-4-fluoro-5-methylphenyl}-1-(cyclopropylmethyl)pyrazol-3-yl)oxy]-2-fluorophenyl}({5-[2-(3-fluoroazetidin-1-yl)ethyl]-2-oxo-4-(trifluoromethyl)pyridin-1-yl})acetic acid (350 mg, 0.45 mmol, 1.00 equiv.), EDCI (129 mg, 0.67 mmol, 1.50 equiv.), HOBT (79 mg, 0.58 mmol, 1.30 equiv.) and DIEA (291 mg, 2.25 mmol, 5.00 equiv.) in MeCN (10 mL) was stirred for 2 h at 60° C. under nitrogen atmosphere and then cooled to room temperature and concentrated. The residue was purified by reversed-phase flash chromatography (C18 gel; mobile phase, MeCN in water (0.1% NH₃·H₂O), 50% to 70% gradient in 10 min; detector, UV 254 nm) to provide ethyl 2-((8S)-21-(cyclopropylmethyl)-14,44-difluoro-5-(5-(2-(3-fluoroazetidin-1-yl)ethyl)-2-oxo-4-(trifluoromethyl)pyridin-1(2H)-yl)-15-methyl-6-oxo-21H-3-oxa-7-aza-2(4,3)-pyrazola-1,4(1,3)-dibenzenacyclooctaphane-8-yl)acetate (50 mg, 14.6%) as a white solid. LC-MS: (ES+H, m/z) [M+H]⁺=758.4.

### Step 4: Preparation of 2-((5S,8S)-21-(cyclopropylmethyl)-14,44-difluoro-5-(5-(2-(3-fluoroazetidin-1-yl)ethyl)-2-oxo-4-(trifluoromethyl)pyridin-1(2H)-yl)-15-methyl-6-oxo-21H-3-oxa-7-aza-2(4,3)-pyrazola-1,4(1,3)-dibenzenacyclooctaphane-8-yl)acetic acid

A mixture of ethyl 2-((8S)-21-(cyclopropylmethyl)-14,44-difluoro-5-(5-(2-(3-fluoroazetidin-1-yl)ethyl)-2-oxo-4-(trifluoromethyl)pyridin-1(2H)-yl)-15-methyl-6-oxo-21H-3-oxa-7-aza-2(4,3)-pyrazola-1,4(1,3)-dibenzenacyclooctaphane-8-yl)acetate (40 mg, 0.05 mmol, 1.00 equiv.) and LiOH·H₂O (6 mg, 0.15 mmol, 3.00 equiv.) in THF/H₂O=5/1 (0.6 mL) was stirred for 2 h at room temperature under nitrogen atmosphere. The resulting mixture was concentrated under reduced pressure and the crude product was purified by Prep-HPLC (Column: Xselect CSH C18 OBD Column 30*150 mm, 5 μm; Mobile Phase A: water (0.1% HCOOH), Mobile Phase B: MeCN; Flow rate: 60 mL/min; Gradient: 17% B to 37% B in 8 min; Wavelength: 254 nm/220 nm; RT1 (min) 8.77) to afford 2-((5S,8S)-21-(cyclopropylmethyl)-14,44-difluoro-5-(5-(2-(3-fluoroazetidin-1-yl)ethyl)-2-oxo-4-(trifluoromethyl)pyridin-1(2H)-yl)-15-methyl-6-oxo-21H-3-oxa-7-aza-2(4,3)-pyrazola-1,4(1,3)-dibenzenacyclooctaphane-8-yl)acetic acid (13.1 mg, 44.5%) as a white solid. LC-MS: (ES+H, m/z) [M+H]⁺=730.20. ¹H NMR (300 MHz, CD₃OD) δ 7.68-7.58 (m, 2H), 7.54 (s, 1H), 7.29 (t, 1H), 7.14-7.04 (m, 2H), 7.00 (s, 1H), 6.89 (s, 1H), 6.60-6.55 (m, 1H), 5.66 (dd, 1H), 5.12-5.02 (m, 1H), 3.99-3.91 (m, 2H), 3.66 (d, 2H), 3.23 (s, 2H), 2.69 (dd, 3H), 2.63-2.48 (m, 3H), 2.30 (d, 3H), 1.34 (td, 1H), 0.70-0.61 (m, 2H), 0.47-0.38 (m, 2H). ¹⁹F NMR (282 MHz, CD₃OD) δ −65.02, −121.73, −127.11, −180.50.

### Example 61

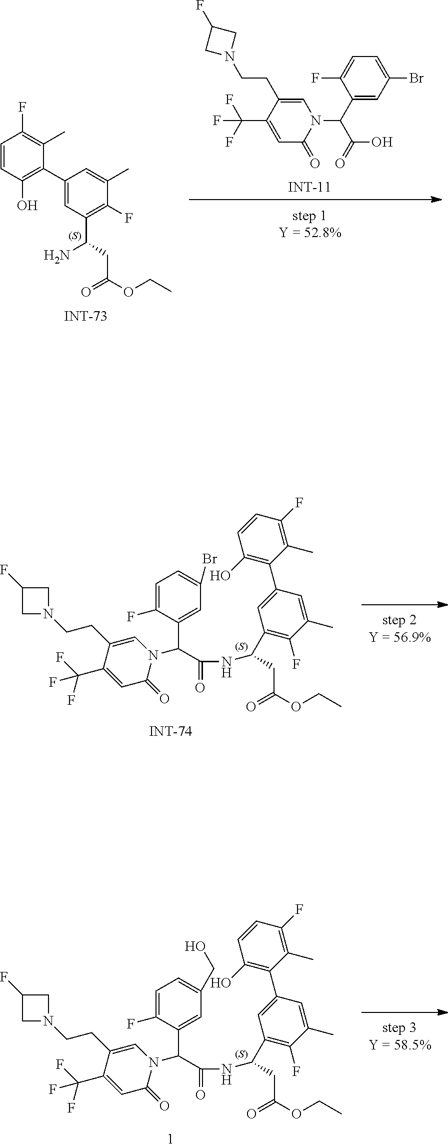

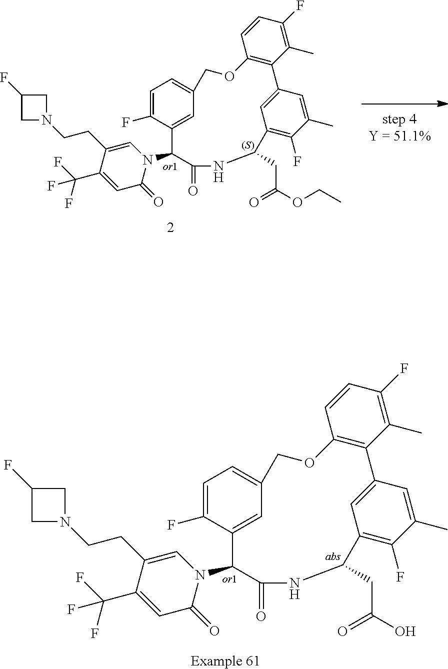

### Step 1: Preparation of ethyl (3S)-3-[2-(5-bromo-2-fluorophenyl)-2-{5-[2-(3-fluoroazetidin-1-yl)ethyl]-2-oxo-4-(trifluoromethyl)pyridin-1-yl}acetamido]-3-{3′,4-difluoro-6′-hydroxy-2′,5-dimethyl-[1,1′-biphenyl]-3-yl}propanoate

A mixture of (5-bromo-2-fluorophenyl)({5-[2-(3-fluoroazetidin-1-yl)ethyl]-2-oxo-4-(trifluoromethyl)pyridin-1-yl})acetic acid (374 mg, 0.75 mmol, 1.10 equiv.), EDCI (395 mg, 2.06 mmol, 3.00 equiv.) and HOBT (278 mg, 2.06 mmol, 3.00 equiv.) in MeCN (5 mL) was stirred for 2 h at 30° C. under nitrogen atmosphere. To the stirred mixture was added ethyl (S)-3-amino-3-(3′,4-difluoro-6′-hydroxy-2′,5-dimethyl-[1,1′-biphenyl]-3-yl)propanoate (240 mg, 0.687 mmol, 1.0 equiv.) and Et₃N (278 mg, 2.74 mmol, 4.00 equiv.) in MeCN (5 mL) dropwise at 0° C. The resulting mixture was stirred overnight at room temperature and then diluted with water (50 mL). The resulting mixture was extracted with EtOAc (3×50 mL) and the combined organic layers were washed with brine (2×20 mL), dried over anhydrous Na₂SO₄, filtered, and concentrated. The residue was purified by reversed-phase flash chromatography (C18 gel; mobile phase, MeCN in water (0.1% NH₃·H₂O), 0% to 100% gradient in 30 min; detector, UV 220 nm) to provide ethyl (3S)-3-[2-(5-bromo-2-fluorophenyl)-2-{5-[2-(3-fluoroazetidin-1-yl)ethyl]-2-oxo-4-(trifluoromethyl)pyridin-1-yl}acetamido]-3-{3′,4-difluoro-6′-hydroxy-2′,5-dimethyl-[1,1′-biphenyl]-3-yl}propanoate (300 mg, 52.83% two steps, mixture) as a white solid. LC-MS: (ES+H, m/z) [M+H]⁺=825.90.

### Step 2: Preparation of ethyl (3S)-3-{3′,4-difluoro-6′-hydroxy-2′,5-dimethyl-[1,1′-biphenyl]-3-yl}-3-{2-[2-fluoro-5-(hydroxymethyl)phenyl]-2-{5-[2-(3-fluoroazetidin-1-yl)ethyl]-2-oxo-4-(trifluoromethyl)pyridin-1-yl}acetamido}propanoate

To a stirred solution of ethyl (3S)-3-[2-(5-bromo-2-fluorophenyl)-2-{5-[2-(3-fluoroazetidin-1-yl)ethyl]-2-oxo-4-(trifluoromethyl)pyridin-1-yl}acetamido]-3-{3′,4-difluoro-6′-hydroxy-2′,5-dimethyl-[1,1′-biphenyl]-3-yl}propanoate (280 mg, 0.33 mmol, 1.00 equiv., mixture) and (tributylstannyl)methanol (543 mg, 1.65 mmol, 5.00 equiv.) in 1,4-dioxane (8.00 mL) was added dicyclohexyl[2′,4′,6′-tris(propan-2-yl)-[1,1′-biphenyl]-2-yl]phosphane; {2′-amino-[1,1′-biphenyl]-2-yl}(chloro)palladium (26 mg, 0.033 mmol, 0.10 equiv.) at room temperature under nitrogen atmosphere. The resulting mixture was stirred for 5 h at 90° C. under nitrogen atmosphere and then cooled to room temperature and concentrated. The residue was purified by reversed-phase flash chromatography (C18 gel; mobile phase, MeCN in water (10 mmol/L NH₄HCO₃), 10% to 90% gradient in 40 min; detector, UV 254/220 nm) to provide ethyl (3S)-3-{3′,4-difluoro-6′-hydroxy-2′,5-dimethyl-[1,1′-biphenyl]-3-yl}-3-{2-[2-fluoro-5-(hydroxymethyl)phenyl]-2-{5-[2-(3-fluoroazetidin-1-yl)ethyl]-2-oxo-4-(trifluoromethyl)pyridin-1-yl}acetamido}propanoate (150 mg, 56.9%, mixture) as a white solid. LC-MS: (ES+H, m/z) [M+H]⁺=778.35. ¹H NMR (400 MHz, DMSO-d₆) δ 9.28 (d, J=8.2 Hz, 1H), 9.11 (s, 1H), 7.64-7.38 (m, 1H), 7.32-7.13 (m, 2H), 7.03 (t, J=6.9 Hz, 2H), 6.95 (td, J=9.2, 6.5 Hz, 2H), 6.82 (d, J=4.3 Hz, 1H), 6.72 (d, J=5.6 Hz, 1H), 5.76 (s, 1H), 5.61 (d, J=9.6 Hz, 1H), 5.28 (dt, J=54.6, 5.5 Hz, 1H), 4.89 (dq, J=58.0, 4.9 Hz, 1H), 4.56 (d, J=4.8 Hz, 1H), 4.32 (dt, J=17.3, 5.1 Hz, 1H), 4.08-3.97 (m, 2H), 3.60-3.36 (m, 1H), 3.32-3.14 (m, 2H), 2.82 (td, J=25.5, 25.1, 5.4 Hz, 4H), 2.42 (dd, J=12.1, 6.7 Hz, 1H), 2.25 (s, 3H), 1.99 (s, 1H), 1.79 (d, J=18.3 Hz, 3H), 1.17 (t, J=7.1 Hz, 1H), 1.12-1.02 (m, 3H).

### Step 3: Preparation of ethyl 2-[(15S,18S)-4,13,20-trifluoro-15-{5-[2-(3-fluoroazetidin-1-yl)ethyl]-2-oxo-4-(trifluoromethyl)pyridin-1-yl}-3,21-dimethyl-16-oxo-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetate

To a stirred solution of ethyl (3S)-3-{3′,4-difluoro-6′-hydroxy-2′,5-dimethyl-[1,1′-biphenyl]-3-yl}-3-{2-[2-fluoro-5-(hydroxymethyl)phenyl]-2-{5-[2-(3-fluoroazetidin-1-yl)ethyl]-2-oxo-4-(trifluoromethyl)pyridin-1-yl}acetamido}propanoate (140 mg, 0.180 mmol, 1.00 equiv., mixture) and 2-(tributyl-lambda5-phosphanylidene)acetonitrile (217 mg, 0.90 mmol, 5.00 equiv.) in toluene (5 mL) was stirred overnight at 100° C. under nitrogen atmosphere and then cooled to room temperature. The resulting mixture was concentrated under reduced pressure. The resulting mixture was diluted with water (30 mL), was extracted with EtOAc (3×50 mL). The combined organic layers were washed with brine (3×30 mL), dried over anhydrous Na₂SO₄, filtered, and concentrated. The residue was purified by reversed-phase flash chromatography (C18 gel; mobile phase, MeCN in water (10 mmol/L NH₄HCO₃), 10% to 90% gradient in 40 min; detector, UV 254/220 nm) to provide ethyl 2-[(15S,18S)-4,13,20-trifluoro-15-{5-[2-(3-fluoroazetidin-1-yl)ethyl]-2-oxo-4-(trifluoromethyl)pyridin-1-yl}-3,21-dimethyl-16-oxo-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetate (80 mg, 58.5%) as a light yellow solid. LC-MS: (ES+H, m/z) [M+H]⁺=760.30. ¹H NMR (300 MHz, CD₃OD) δ 7.52 (s, 1H), 7.31 (d, J=7.4 Hz, 1H), 7.18 (d, J=11.1 Hz, 3H), 7.03 (d, J=9.9 Hz, 3H), 6.88 (s, 1H), 6.65 (d, J=7.4 Hz, 1H), 5.79 (dd, J=9.2, 5.9 Hz, 1H), 5.51 (s, 1H), 5.31 (d, J=13.1 Hz, 1H), 5.03 (d, J=13.3 Hz, 2H), 4.17 (tq, J=7.1, 3.8 Hz, 2H), 3.43 (dt, J=16.1, 8.3 Hz, 2H), 2.85-2.71 (m, 2H), 2.62-2.51 (m, 2H), 2.46 (d, J=7.2 Hz, 2H), 2.35 (d, J=2.1 Hz, 3H), 1.91 (d, J=2.5 Hz, 3H), 1.23 (t, J=7.1 Hz, 3H).

### Step 4: Preparation of [(15S,18S)-4,13,20-trifluoro-15-{5-[2-(3-fluoroazetidin-1-yl)ethyl]-2-oxo-4-(trifluoromethyl)pyridin-1-yl}-3,21-dimethyl-16-oxo-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetic acid

To a stirred solution of ethyl 2-[(15S,18S)-4,13,20-trifluoro-15-{5-[2-(3-fluoroazetidin-1-yl)ethyl]-2-oxo-4-(trifluoromethyl)pyridin-1-yl}-3,21-dimethyl-16-oxo-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetate (80 mg, 0.10 mmol, 1.00 equiv.) in THF (2 mL) and H₂O (0.5 mL) was added LiOH·H₂O (8 mg, 0.31 mmol, 3.00 equiv.) at room temperature under nitrogen atmosphere. The resulting mixture was stirred for 2 h at room temperature and then neutralized to pH 6 with 1 M HCl (aq.) and concentrated. The residue was dissolved in THF (2 mL) and purified by Prep-HPLC (Column: XBridge Prep OBD C18 Column, 30*150 mm, 5 μm; Mobile Phase A: water (10 mmol/L NH₄HCO₃, Mobile Phase B: MeCN; Flow rate: 60 mL/min; Gradient: 30% B to 45% B in 10 min; Wavelength: 254 nm/220 nm; RT1 (min) 9.08) to afford [(15S,18S)-4,13,20-trifluoro-15-{5-[2-(3-fluoroazetidin-1-yl)ethyl]-2-oxo-4-(trifluoromethyl)pyridin-1-yl}-3,21-dimethyl-16-oxo-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetic acid (40 mg, 51.1%) as a white solid. LC-MS: (ES+H, m/z) [M+H]⁺=732.20. 1H NMR (400 MHz, CD₃OD) δ 7.47 (ddd, J=7.8, 4.9, 2.2 Hz, 1H), 7.31 (dd, J=7.2, 2.1 Hz, 1H), 7.21 (s, 1H), 7.16-7.06 (m, 3H), 7.01-6.92 (m, 2H), 6.86 (s, 1H), 6.69 (dd, J=6.7, 2.2 Hz, 1H), 5.73 (dd, J=8.5, 5.6 Hz, 1H), 5.27 (d, J=13.1 Hz, 1H), 5.07-4.89 (m, 2H), 3.61 (dtd, J=17.1, 6.0, 2.9 Hz, 2H), 3.30-3.17 (m, 2H), 2.75-2.59 (m, 4H), 2.47 (h, J=8.4, 7.7 Hz, 2H), 2.31 (d, J=1.9 Hz, 3H), 1.89 (d, J=2.4 Hz, 3H). 19F NMR (377 MHz, CD₃OD) δ −64.90, −118.05, −127.00, −127.40, −180.44.

### Example 62

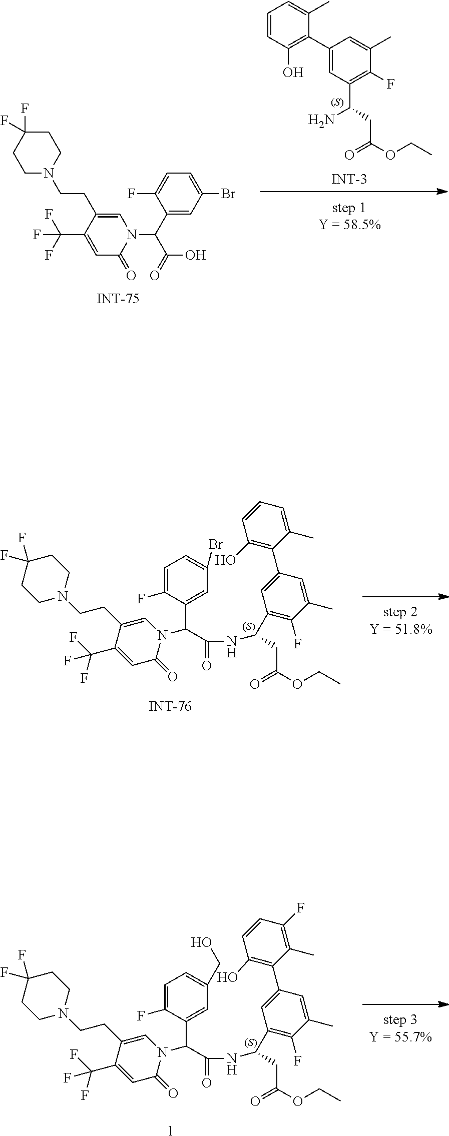

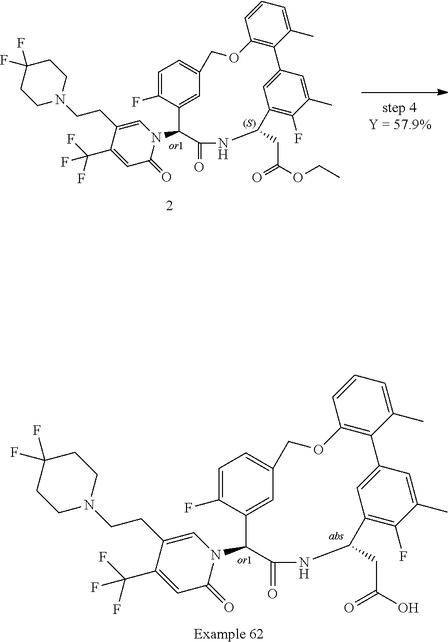

### Step 1: Preparation of ethyl (3S)-3-[2-(5-bromo-2-fluorophenyl)-2-{5-[2-(4,4-difluoropiperidin-1-yl)ethyl]-2-oxo-4-(trifluoromethyl)pyridin-1-yl}acetamido]-3-{4-fluoro-2′-hydroxy-5,6′-dimethyl-[1,1′-biphenyl]-3-yl}propanoate

To a stirred mixture of (5-bromo-2-fluorophenyl)({5-[2-(4,4-difluoropiperidin-1-yl)ethyl]-2-oxo-4-(trifluoromethyl)pyridin-1-yl})acetic acid (800 mg, 1.47 mmol, 1.00 equiv.) in MeCN (5 mL) were added EDCI (566 mg, 2.95 mmol, 2.00 equiv.) and HOBT (399 mg, 2.95 mmol, 2.00 equiv.) at room temperature under nitrogen atmosphere. The resulting mixture was stirred for 1 h at room temperature and then a solution of ethyl (3S)-3-amino-3-{4-fluoro-2′-hydroxy-5,6′-dimethyl-[1,1′-biphenyl]-3-yl}propanoate (832 mg, 2.51 mmol, 1.70 equiv.) and Et₃N (897 mg, 8.86 mmol, 6.00 equiv.) in MeCN (5 mL) was added portions over 1 min at room temperature. The resulting mixture was stirred overnight at room temperature and then concentrated. The residue was purified by reversed-phase flash chromatography (C18 gel; mobile phase, MeCN in water (0.1% NH₃·H₂O), 40% to 60% gradient in 20 min; detector, UV 254/220 nm) to afford ethyl (3S)-3-[2-(5-bromo-2-fluorophenyl)-2-{5-[2-(4,4-difluoropiperidin-1-yl)ethyl]-2-oxo-4-(trifluoromethyl)pyridin-1-yl}acetamido]-3-{4-fluoro-2′-hydroxy-5,6′-dimethyl-[1,1′-biphenyl]-3-yl}propanoate (740 mg, 58.5%, mixture) as a yellow oil. LC-MS: (ES+H, m/z) [M+H]⁺=854.2.

### Step 2: Preparation of ethyl (3S)-3-(2-{5-[2-(4,4-difluoropiperidin-1-yl)ethyl]-2-oxo-4-(trifluoromethyl)pyridin-1-yl}-2-[2-fluoro-5-(hydroxymethyl)phenyl]acetamido)-3-{4-fluoro-2′-hydroxy-5,6′-dimethyl-[1,1′-biphenyl]-3-yl}propanoate

To a stirred mixture of ethyl (3S)-3-[2-(5-bromo-2-fluorophenyl)-2-{5-[2-(4,4-difluoropiperidin-1-yl)ethyl]-2-oxo-4-(trifluoromethyl)pyridin-1-yl}acetamido]-3-{4-fluoro-2′-hydroxy-5,6′-dimethyl-[1,1′-biphenyl]-3-yl}propanoate (450 mg, 0.52 mmol, 1.00 equiv.) in 1,4-dioxane (10 mL) was added (tributylstannyl)methanol (507 mg, 1.58 mmol, 3.00 equiv.) and XPhos-Pd-2nd Gen (25 mg, 0.05 mmol, 0.10 equiv.) at room temperature under nitrogen atmosphere. The resulting mixture was stirred overnight at 90° C. under nitrogen atmosphere and then cooled to room temperature. The reaction was diluted with water (20 mL) and extracted with EtOAc (2×20 mL). The combined organic layers were washed with brine (2×20 mL), dried over anhydrous Na₂SO₄, filtered, and concentrated. The residue was purified by reversed-phase flash chromatography (C18 gel; mobile phase, MeCN in water (0.1% NH₃·H₂O), 40% to 60% gradient in 20 min; detector, UV 254/220 nm) to afford ethyl (3S)-3-(2-{5-[2-(4,4-difluoropiperidin-1-yl)ethyl]-2-oxo-4-(trifluoromethyl)pyridin-1-yl}-2-[2-fluoro-5-(hydroxymethyl)phenyl]acetamido)-3-{4-fluoro-2′-hydroxy-5,6′-dimethyl-[1,1′-biphenyl]-3-yl}propanoate (220 mg, 51.8%, mixture) as colorless oil. LC-MS: (ES+H, m/z) [M+H]⁺=806.5.

### Step 3: Preparation of ethyl 2-[(15S,18S)-15-{5-[2-(4,4-difluoropiperidin-1-yl)ethyl]-2-oxo-4-(trifluoromethyl)pyridin-1-yl}-13,20-difluoro-3,21-dimethyl-16-oxo-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetate

To a stirred mixture of ethyl (3S)-3-(2-{5-[2-(4,4-difluoropiperidin-1-yl)ethyl]-2-oxo-4-(trifluoromethyl)pyridin-1-yl}-2-[2-fluoro-5-(hydroxymethyl)phenyl]acetamido)-3-{4-fluoro-2′-hydroxy-5,6′-dimethyl-[1,1′-biphenyl]-3-yl}propanoate (220 mg, 0.27 mmol, 1.00 equiv.) in Toluene (5 mL) was added 2-(tributyl-lambda5-phosphanylidene)acetonitrile (329 mg, 1.36 mmol, 5.00 equiv.) at room temperature under nitrogen atmosphere. The resulting mixture was stirred overnight at 100° C. and then cooled to room temperature and concentrated. The residue was purified by reversed-phase flash chromatography (C18 gel; mobile phase, MeCN in water (0.1% NH₃·H₂O), 40% to 60% gradient in 20 min; detector, UV 254/220 nm) to afford ethyl 2-[(15S,18S)-15-{5-[2-(4,4-difluoropiperidin-1-yl)ethyl]-2-oxo-4-(trifluoromethyl)pyridin-1-yl}-13,20-difluoro-3,21-dimethyl-16-oxo-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetate (120 mg, 55.7%) as a yellow solid. LC-MS: (ES+H, m/z) [M+H]⁺=788.4.

### Step 4: Preparation of [(15S,18S)-15-{5-[2-(4,4-difluoropiperidin-1-yl)ethyl]-2-oxo-4-(trifluoromethyl)pyridin-1-yl}-13,20-difluoro-3,21-dimethyl-16-oxo-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetic acid

To a stirred mixture of ethyl 2-[(15S,18S)-15-{5-[2-(4,4-difluoropiperidin-1-yl)ethyl]-2-oxo-4-(trifluoromethyl)pyridin-1-yl}-13,20-difluoro-3,21-dimethyl-16-oxo-8-oxa-17-azatetracyclo [17.3.1.1\hat{ }{10,14}.0\hat{ }{2,7}]tetracosa-1(23),2(7),3,5,10,12,14(24),19,21-nonaen-18-yl]acetate (130 mg, 0.16 mmol, 1.00 equiv.) in THF (5 mL) was added LiOH·H₂O (14 mg, 0.33 mmol, 2.00 equiv.) in H₂O (2.5 mL) at room temperature under nitrogen atmosphere. The resulting mixture was stirred for 1 h at room temperature and then diluted with water (5 mL). The reaction mixture was acidified to pH 5 with 1 M HCl (aq.) and concentrated. The crude product was purified by Prep-HPLC (Column: XBridge Shield RP18 OBD Column, 30*150 mm, 5 μm; Mobile Phase A: water (10 mmol/L NH₄HCO₃), Mobile Phase B: MeCN; Flow rate: 60 mL/min; Gradient: 30% B to 45% B in 10 min; Wavelength: 254 nm/220 nm; RT1 (min) 9.1) to afford [(15S,18S)-15-{5-[2-(4,4-difluoropiperidin-1-yl)ethyl]-2-oxo-4-(trifluoromethyl)pyridin-1-yl}-13,20-difluoro-3,21-dimethyl-16-oxo-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetic acid (74 mg, 57.9%) as a white solid. LC-MS: (ES+H, m/z) [M+H]⁺=760.35. ¹H NMR (400 MHz, DMSO-d₆) δ 9.56 (d, J=7.2 Hz, 1H), 7.53-7.46 (m, 1H), 7.30 (d, J=8.4 Hz, 1H), 7.23 (s, 1H), 7.21-7.14 (m, 3H), 7.04 (s, 1H), 6.97 (d, J=7.0, 2.1 Hz, 1H), 6.82 (d, J=6.7 Hz, 2H), 6.45 (d, J=6.8, 2.2 Hz, 1H), 5.59-5.48 (m, 1H), 5.36 (d, J=13.1 Hz, 1H), 4.97 (d, J=13.2 Hz, 1H), 2.45 (d, J=6.8 Hz, 3H), 2.42-2.30 (m, 4H), 2.30-2.19 (m, 6H), 1.89 (s, 3H), 1.67-1.50 (m, 4H). ¹⁹F NMR (377 MHz, DMSO-d₆) δ −62.57, −95.89, −117.00, −127.62.

### Example 63

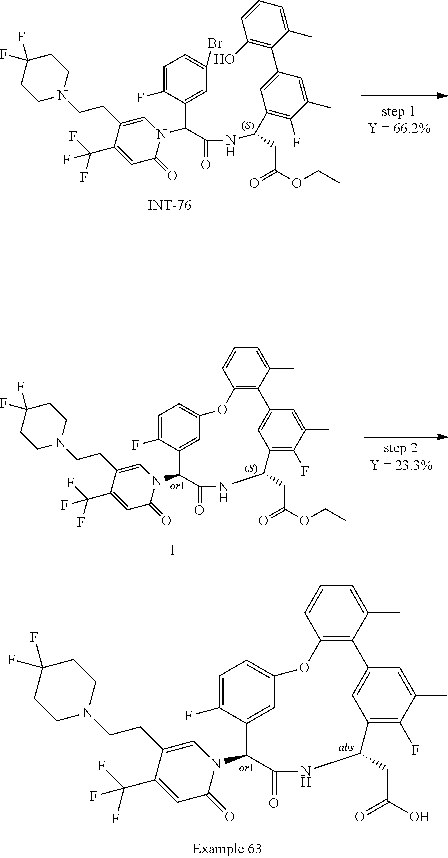

### Step 1: Preparation of ethyl 2-[(14S,17S)-14-{5-[2-(4,4-difluoropiperidin-1-yl)ethyl]-2-oxo-4-(trifluoromethyl)pyridin-1-yl}-12,19-difluoro-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate

To a stirred mixture of ethyl (3S)-3-[2-(5-bromo-2-fluorophenyl)-2-{5-[2-(4,4-difluoropiperidin-1-yl)ethyl]-2-oxo-4-(trifluoromethyl)pyridin-1-yl}acetamido]-3-{4-fluoro-2′-hydroxy-5,6′-dimethyl-[1,1′-biphenyl]-3-yl}propanoate (250 mg, 0.29 mmol, 1.00 equiv.) in 1,4-dioxane (8 mL) were added CuI (27 mg, 0.14 mmol, 0.50 equiv.), K₂CO₃ (80 mg, 0.58 mmol, 2.00 equiv.) and (1R,2R)—N1,N2-dimethylcyclohexane-1,2-diamine (41 mg, 0.29 mmol, 1.00 equiv.) at room temperature under nitrogen atmosphere. The resulting mixture was stirred overnight at 100° C. under nitrogen atmosphere and then cooled to room temperature and concentrated. The residue was purified by reversed-phase flash chromatography (C18 gel; mobile phase, MeCN in water (0.1% NH₃·H₂O), 50% to 70% gradient in 20 min; detector, UV 220 nm) to provide ethyl 2-[(14S,17S)-14-{5-[2-(4,4-difluoropiperidin-1-yl)ethyl]-2-oxo-4-(trifluoromethyl)pyridin-1-yl}-12,19-difluoro-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (150 mg, 66.2%) as a colorless oil. LC-MS: (ES+H, m/z) [M+H]⁺=774.2.

### Step 2: Preparation of [(14S,17S)-14-{5-[2-(4,4-difluoropiperidin-1-yl)ethyl]-2-oxo-4-(trifluoromethyl)pyridin-1-yl}-12,19-difluoro-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid

To a stirred mixture of ethyl 2-[(14S,17S)-14-{5-[2-(4,4-difluoropiperidin-1-yl)ethyl]-2-oxo-4-(trifluoromethyl)pyridin-1-yl}-12,19-difluoro-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (120 mg, 0.16 mmol, 1.00 equiv.) in THF (5 mL) was added LiOH·H₂O (13 mg, 0.31 mmol, 2.00 equiv.) in H₂O (2.50 mL) at room temperature under nitrogen atmosphere. The resulting mixture was stirred for 1 h at room temperature and then diluted with water (5 mL). The reaction mixture was acidified to pH 5 with 1.00 M HCl (aq.) and then concentrated. The crude product was purified by Prep-HPLC (Column: XBridge Shield RP18 OBD Column, 30*150 mm, 5 μm; Mobile Phase A: water (10 mmol/L NH₄HCO₃+0.05% NH₃·H₂O), Mobile Phase B: MeCN; Flow rate: 60 mL/min; Gradient: 35% B to 50% B in 10 min; Wavelength: 254 nm/220 nm; RT1 (min) 8.2) to afford [(14S,17S)-14-{5-[2-(4,4-difluoropiperidin-1-yl)ethyl]-2-oxo-4-(trifluoromethyl)pyridin-1-yl}-12,19-difluoro-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid (25.8 mg, 20.3%) as a white solid. LC-MS: (ES+H, m/z) [M+H]⁺=746.30. ¹H NMR (400 MHz, CD₃OD) δ 7.55-7.48 (m, 1H), 7.40 (d, J=5.4 Hz, 2H), 7.33 (t, J=7.8 Hz, 1H), 7.21-7.11 (m, 2H), 6.94 (d, J=7.2, 2.1 Hz, 1H), 6.87 (d, J=5.1 Hz, 2H), 6.45 (dd, J=6.3, 2.2 Hz, 1H), 6.14 (dd, J=6.1, 3.0 Hz, 1H), 5.70 (t, J=7.4 Hz, 1H), 2.76-2.53 (m, 4H), 2.52-2.35 (m, 5H), 2.33 (d, J=1.9 Hz, 4H), 1.99 (s, 3H), 1.77-1.62 (m, 4H). ¹⁹F NMR (377 MHz, CD₃OD) δ −65.15, −100.18, −123.29, −126.91.

### Example 64

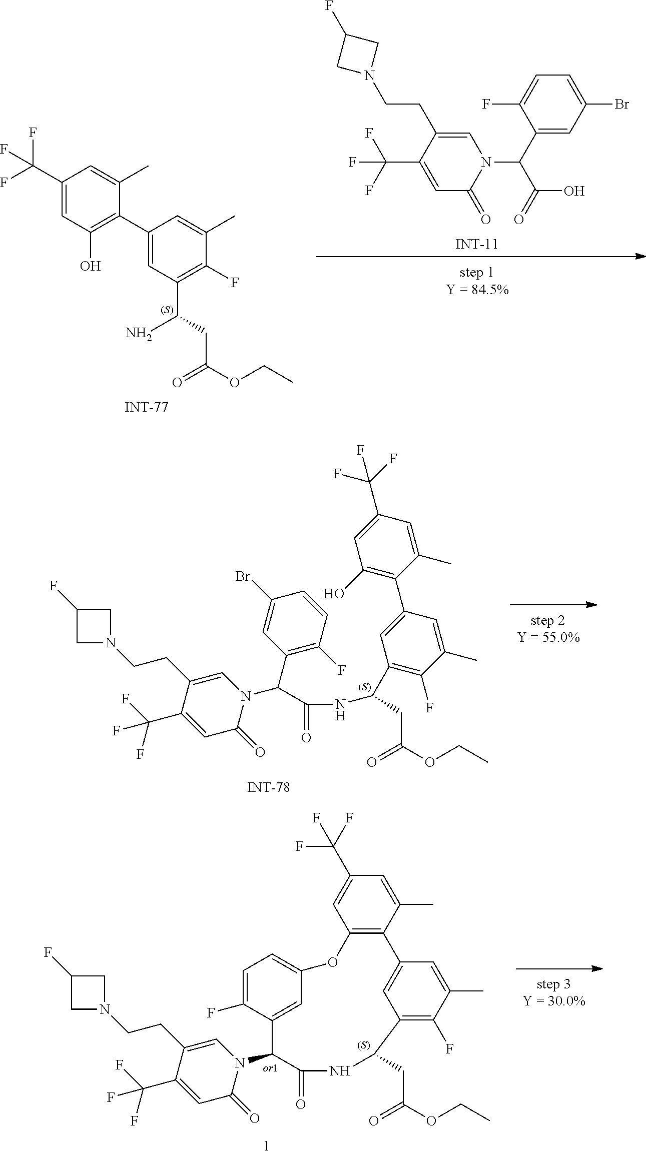

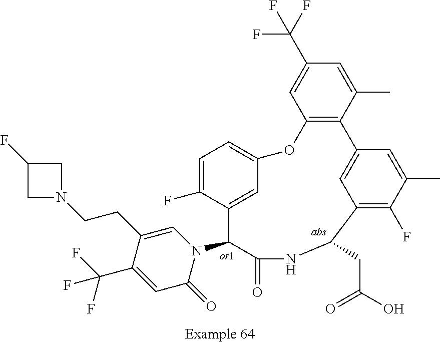

### Step 1: Preparation of ethyl (3S)-3-[2-(5-bromo-2-fluorophenyl)-2-{5-[2-(3-fluoroazetidin-1-yl)ethyl]-2-oxo-4-(trifluoromethyl)pyridin-1-yl}acetamido]-3-[4-fluoro-2′-hydroxy-5,6′-dimethyl-4′-(trifluoromethyl)-[1,1′-biphenyl]-3-yl]propanoate

A mixture of ethyl (3S)-3-amino-3-[4-fluoro-2′-hydroxy-5,6′-dimethyl-4′-(trifluoromethyl)-[1,1′-biphenyl]-3-yl]propanoate (687 mg, 1.721 mmol, 1.20 equiv.) and NEt₃ (870 mg, 8.60 mmol, 6.00 equiv.) in MeCN (6 mL) was stirred for 30 min at 40° C. under nitrogen atmosphere. The resulting mixture was concentrated under reduced pressure and redissolved in MeCN (2 mL). In a separate vial, a mixture of (5-bromo-2-fluorophenyl)({5-[2-(3-fluoroazetidin-1-yl)ethyl]-2-oxo-4-(trifluoromethyl)pyridin-1-yl})acetic acid (710 mg, 1.43 mmol, 1.00 equiv.), HOBT (252 mg, 1.86 mmol, 1.30 equiv.) and EDCI (412 mg, 2.15 mmol, 1.50 equiv.) in MeCN (5 mL) was stirred for 1 h at 40° C. under nitrogen atmosphere. To the above mixture was added ethyl (3S)-3-amino-3-[4-fluoro-2′-hydroxy-5,6′-dimethyl-4′-(trifluoromethyl)-[1,1′-biphenyl]-3-yl]propanoate in MeCN (2 mL) at room temperature. The resulting mixture was stirred overnight at 40° C. and then concentrated. The residue was purified by reversed-phase flash chromatography (C18 gel; mobile phase, MeCN in water (0.1% NH₃·H₂O), 0% to 80% gradient in 30 min; detector, UV 254 nm) to afford ethyl (3S)-3-[2-(5-bromo-2-fluorophenyl)-2-{5-[2-(3-fluoroazetidin-1-yl)ethyl]-2-oxo-4-(trifluoromethyl)pyridin-1-yl}acetamido]-3-[4-fluoro-2′-hydroxy-5,6′-dimethyl-4′-(trifluoromethyl)-[1,1′-biphenyl]-3-yl]propanoate (1.06 g, 84.5%) as a white solid. LC-MS: (ES+H, m/z) [M+H]⁺=875.9. ¹H NMR (300 MHz, DMSO-d₆) δ 9.87 (s, 1H), 7.53 (dd, J=6.5, 2.6 Hz, 1H), 7.40-7.21 (m, 2H), 7.19 (d, J=5.6 Hz, 1H), 7.05 (d, J=4.2 Hz, 2H), 6.83 (d, J=3.5 Hz, 2H), 6.71 (s, 1H), 5.80-5.51 (m, 2H), 4.82 (t, J=5.4 Hz, 1H), 4.16-4.04 (m, 2H), 3.18 (d, J=5.2 Hz, 2H), 2.99-2.68 (m, 4H), 2.43 (s, 3H), 2.37 (d, J=13.3 Hz, 2H), 2.31-2.23 (m, 3H), 1.99 (s, 3H), 1.08 (t, J=7.0 Hz, 3H). ¹⁹F NMR (282 MHz, DMSO-d₆) δ −60.92, −62.73, −116.93, −125.79, −176.82.

### Step 2: Preparation of ethyl 2-[(14S,17S)-12,19-difluoro-14-{5-[2-(3-fluoroazetidin-1-yl)ethyl]-2-oxo-4-(trifluoromethyl)pyridin-1-yl}-3,20-dimethyl-15-oxo-5-(trifluoromethyl)-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate

A mixture of ethyl (3S)-3-[2-(5-bromo-2-fluorophenyl)-2-{5-[2-(3-fluoroazetidin-1-yl)ethyl]-2-oxo-4-(trifluoromethyl)pyridin-1-yl}acetamido]-3-[4-fluoro-2′-hydroxy-5,6′-dimethyl-4′-(trifluoromethyl)-[1,1′-biphenyl]-3-yl]propanoate (80 mg, 0.09 mmol, 1.00 equiv.), K₂CO₃ (25 mg, 0.18 mmol, 2.00 equiv.), CuI (8 mg, 0.05 mmol, 0.50 equiv.) and (1R,2R)—N1,N2-dimethylcyclohexane-1,2-diamine (12 mg, 0.09 mmol, 1.00 equiv.) in 1,4-dioxane (3 mL) was stirred overnight at 100° C. under nitrogen atmosphere. The resulting mixture was concentrated under reduced pressure and the crude product was purified by reversed-phase flash chromatography (C18 gel; mobile phase, MeCN in water (0.1% NH₃·H₂O), 10% to 50% gradient in 30 min; detector, UV 220 nm) to provide ethyl 2-[(14S,17S)-12,19-difluoro-14-{5-[2-(3-fluoroazetidin-1-yl)ethyl]-2-oxo-4-(trifluoromethyl)pyridin-1-yl}-3,20-dimethyl-15-oxo-5-(trifluoromethyl)-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (40 mg, 55.0%) as a white solid. LC-MS: (ES+H, m/z) [M+H]⁺=796.25. ¹H NMR (400 MHz, CD₃OD) δ 7.81 (d, J=9.0 Hz, 1H), 7.57 (dt, J=9.1, 3.6 Hz, 1H), 7.50 (d, J=7.2 Hz, 1H), 7.33 (s, 1H), 7.21 (t, J=9.0 Hz, 1H), 7.04 (d, J=7.0 Hz, 1H), 6.88 (d, J=10.6 Hz, 1H), 6.81 (d, J=4.5 Hz, 1H), 6.51-6.43 (m, 1H), 6.13-6.06 (m, 1H), 5.70 (t, J=7.9 Hz, 1H), 5.01-4.81 (m, 1H), 4.11 (qt, J=6.9, 3.5 Hz, 2H), 3.48-3.37 (m, 2H), 3.08-2.96 (m, 2H), 2.87 (dd, J=15.4, 8.3 Hz, 1H), 2.74 (dd, J=15.5, 7.6 Hz, 1H), 2.59-2.42 (m, 4H), 2.36 (s, 3H), 2.08 (s, 3H), 1.19 (t, J=7.1 Hz, 3H). ¹⁹FNMR (377 MHz, CD₃OD) δ −63.97, −65.04, −122.41, −125.68, −180.21.

### Step 3: Preparation of [(14S,17S)-12,19-difluoro-14-{5-[2-(3-fluoroazetidin-1-yl)ethyl]-2-oxo-4-(trifluoromethyl)pyridin-1-yl}-3,20-dimethyl-15-oxo-5-(trifluoromethyl)-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid

A solution of ethyl 2-[(14S,17S)-12,19-difluoro-14-{5-[2-(3-fluoroazetidin-1-yl)ethyl]-2-oxo-4-(trifluoromethyl)pyridin-1-yl}-3,20-dimethyl-15-oxo-5-(trifluoromethyl)-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (40 mg, 0.05 mmol, 1.00 equiv.) and LiOH·H₂O (3 mg, 0.15 mmol, 3.00 equiv.) in THF (2 mL) and H₂O (0.5 mL) was stirred overnight at room temperature under nitrogen atmosphere. The mixture was then acidified to pH 5 with 1 M HCl (aq.) and concentrated. The crude product was purified by Prep-HPLC (Column: XBridge Prep OBD C18 Column, 30*150 mm, 10 μm; Mobile Phase A: water (10 mmol/L NH₄HCO₃+0.05% NH₃H₂O), Mobile Phase B: MeCN; Flow rate: 60 mL/min; Gradient: 30% B to 50% B in 8 min; Wavelength: 254/220 nm) to afford [(14S,17S)-12,19-difluoro-14-{5-[2-(3-fluoroazetidin-1-yl)ethyl]-2-oxo-4-(trifluoromethyl)pyridin-1-yl}-3,20-dimethyl-15-oxo-5-(trifluoromethyl)-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid (12 mg, 30.0%) as a white solid. LC-MS: (ES+H, m/z) [M+H]⁺=768.20. ¹H NMR (400 MHz, DMSO-d₆) δ 9.39 (s, 1H), 8.10 (s, 1H), 7.72 (dt, J=9.0, 3.6 Hz, 1H), 7.61 (s, 1H), 7.31 (t, J=9.2 Hz, 1H), 7.22 (s, 1H), 7.06 (d, J=6.7 Hz, 1H), 6.80 (s, 1H), 6.69 (s, 1H), 6.48 (dd, J=6.3, 2.2 Hz, 1H), 5.99 (dd, J=6.2, 3.0 Hz, 1H), 5.45 (q, J=7.2 Hz, 1H), 4.85 (dt, J=57.8, 5.2 Hz, 1H), 2.85 (ddd, J=20.5, 8.5, 4.3 Hz, 2H), 2.54 (s, 2H), 2.45-2.37 (m, 2H), 2.36-2.27 (m, 7H), 2.03 (s, 3H). ¹⁹F NMR (377 MHz, DMSO-d₆) δ −60.72, −62.65, −121.06, −125.05, −176.82.

### Example 65

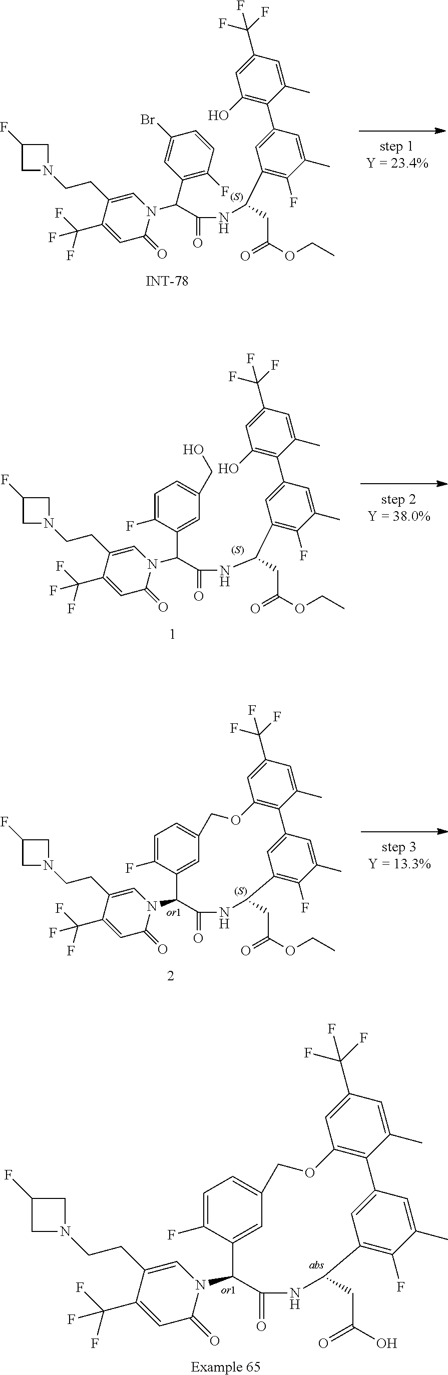

### Step 1: Preparation of ethyl (3S)-3-[4-fluoro-2′-hydroxy-5,6′-dimethyl-4′-(trifluoromethyl)-[1,1′-biphenyl]-3-yl]-3-{2-[2-fluoro-5-(hydroxymethyl)phenyl]-2-{5-[2-(3-fluoroazetidin-1-yl)ethyl]-2-oxo-4-(trifluoromethyl)pyridin-1-yl}acetamido}propanoate

To a stirred solution of ethyl (3S)-3-[2-(5-bromo-2-fluorophenyl)-2-{5-[2-(3-fluoroazetidin-1-yl)ethyl]-2-oxo-4-(trifluoromethyl)pyridin-1-yl}acetamido]-3-[4-fluoro-2′-hydroxy-5,6′-dimethyl-4′-(trifluoromethyl)-[1,1′-biphenyl]-3-yl]propanoate (550 mg, 0.63 mmol, 1.00 equiv.) and (tributylstannyl)methanol (806 mg, 2.51 mmol, 4.00 equiv.) in 1,4-dioxane (10 mL) were added dicyclohexyl[2′,4′,6′-tris(propan-2-yl)-[1,1′-biphenyl]-2-yl]phosphane; {2′-amino-[1,1′-biphenyl]-2-yl}(chloro)palladium (49 mg, 0.06 mmol, 0.10 equiv.) in portions at room temperature under nitrogen atmosphere. The resulting mixture was stirred overnight at 80° C. and then cooled to room temperature and concentrated. The residue was purified by reversed-phase flash chromatography (C18 gel; mobile phase, MeCN in water (0.1% NH₃·H₂O), 0% to 90% gradient in 30 min; detector, UV 220 nm) to afford ethyl (3S)-3-[4-fluoro-2′-hydroxy-5,6′-dimethyl-4′-(trifluoromethyl)-[1,1′-biphenyl]-3-yl]-3-{2-[2-fluoro-5-(hydroxymethyl)phenyl]-2-{5-[2-(3-fluoroazetidin-1-yl)ethyl]-2-oxo-4-(trifluoromethyl)pyridin-1-yl}acetamido}propanoate (126 mg, 23.4%, mixture) as light yellow solid. LC-MS: (ES+H, m/z) [M+H]⁺=828.30.

### Step 2: Preparation of ethyl 2-[(15R*,18S)-13,20-difluoro-15-{5-[2-(3-fluoroazetidin-1-yl)ethyl]-2-oxo-4-(trifluoromethyl)pyridin-1-yl}-3,21-dimethyl-16-oxo-5-(trifluoromethyl)-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetate

A solution of ethyl (3S)-3-[4-fluoro-2′-hydroxy-5,6′-dimethyl-4′-(trifluoromethyl)-[1,1′-biphenyl]-3-yl]-3-{2-[2-fluoro-5-(hydroxymethyl)phenyl]-2-{5-[2-(3-fluoroazetidin-1-yl)ethyl]-2-oxo-4-(trifluoromethyl)pyridin-1-yl}acetamido}propanoate (121 mg, 0.15 mmol, 1.00 equiv.) and 2-(tributyl-lambda5-phosphanylidene)acetonitrile (176 mg, 0.73 mmol, 5.00 equiv.) in toluene (2 mL) was stirred overnight at 100° C. under nitrogen atmosphere and then cooled to room temperature. The resulting mixture was concentrated and then diluted with EtOAc (100 mL). The combined organic layers were washed with brine (3×30 mL), dried over anhydrous Na₂SO₄, filtered, and concentrated. The residue was purified by silica gel column chromatography (petroleum ether/EtOAc (30% to 70% in 20 min) to afford ethyl 2-[(15R*,18S)-13,20-difluoro-15-{5-[2-(3-fluoroazetidin-1-yl)ethyl]-2-oxo-4-(trifluoromethyl)pyridin-1-yl}-3,21-dimethyl-16-oxo-5-(trifluoromethyl)-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetate (45 mg, 38.0%) as a white solid. LC-MS: (ES+H, m/z) [M+H]⁺=810.30.

### Step 3: Preparation of [(15R*,18S)-13,20-difluoro-15-{5-[2-(3-fluoroazetidin-1-yl)ethyl]-2-oxo-4-(trifluoromethyl)pyridin-1-yl}-3,21-dimethyl-16-oxo-5-(trifluoromethyl)-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetic acid

A solution of ethyl 2-[(15R*,18S)-13,20-difluoro-15-{5-[2-(3-fluoroazetidin-1-yl)ethyl]-2-oxo-4-(trifluoromethyl)pyridin-1-yl}-3,21-dimethyl-16-oxo-5-(trifluoromethyl)-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetate (40 mg, 0.05 mmol, 1.00 equiv.) and LiOH·H₂O (8 mg, 0.20 mmol, 4.00 equiv.) in THF (2 mL) and H₂O (0.5 mL) was stirred overnight at room temperature under nitrogen atmosphere and then concentrated. The crude product was purified by Prep-HPLC (Column: Xbridge Phenyl C18 Column, 30*150 mm, 5 μm; Mobile Phase A: water (10 mmol/L NH₄HCO₃), Mobile Phase B: CH₃CN; Flow rate: 60 mL/min; Gradient: 30% B to 45% B in 15 min; Wavelength: 254 nm/220 nm; RT1 (min) 8.38) to afford [(15R*,18S)-13,20-difluoro-15-{5-[2-(3-fluoroazetidin-1-yl)ethyl]-2-oxo-4-(trifluoromethyl)pyridin-1-yl}-3,21-dimethyl-16-oxo-5-(trifluoromethyl)-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetic acid (5.4 mg, 13.3%) as a white solid. LC-MS: (ES+H, m/z) [M+H]⁺=782.25. ¹H NMR (300 MHz, CD₃OD) δ 7.50 (t, J=6.0 Hz, 1H), 7.41 (s, 1H), 7.33 (d, J=7.1 Hz, 1H), 7.22-7.13 (m, 3H), 7.09 (d, J=12.1 Hz, 1H), 6.98 (d, J=7.0 Hz, 1H), 6.86 (s, 1H), 6.72 (d, J=6.6 Hz, 1H), 5.72 (t, J=6.9 Hz, 1H), 5.40 (d, J=13.3 Hz, 1H), 5.15-4.99 (m, 1H), 3.54 (m, 2H), 3.57-3.39 (m, 2H), 3.25-2.97 (m, 1H), 2.70 (t, J=5.9 Hz, 2H), 2.62-2.47 (m, 1H), 2.45 (d, J=7.3 Hz, 2H), 2.33 (s, 3H), 2.24 (d, J=7.1 Hz, 1H), 2.06 (s, 3H). ¹⁹F NMR (282 MHz, CD₃OD) δ −64.02, −64.96, −117.83, −127.01, −180.44.

### Example 66

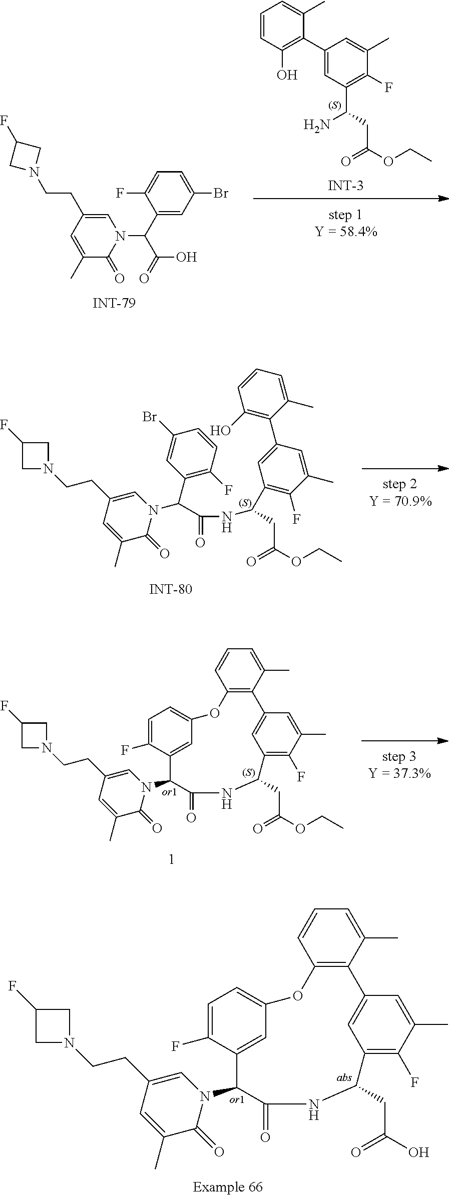

### Step 1: Preparation of ethyl (3S)-3-[2-(5-bromo-2-fluorophenyl)-2-{5-[2-(3-fluoroazetidin-1-yl)ethyl]-3-methyl-2-oxopyridin-1-yl}acetamido]-3-{4-fluoro-2′-hydroxy-5,6′-dimethyl-[1,1′-biphenyl]-3-yl}propanoate

A mixture of (5-bromo-2-fluorophenyl)({5-[2-(3-fluoroazetidin-1-yl)ethyl]-3-methyl-2-oxopyridin-1-yl})acetic acid (580 mg, 1.31 mmol, 1.00 equiv.), EDCI (755 mg, 3.93 mmol, 3.00 equiv.) and HOBT (532 mg, 3.93 mmol, 3.00 equiv.) in MeCN (10 mL) was stirred for 2 h at 30° C. under nitrogen atmosphere. To the stirred reaction mixture was added ethyl (3S)-3-amino-3-{4-fluoro-2′-hydroxy-5,6′-dimethyl-[1,1′-biphenyl]-3-yl}propanoate (479 mg, 1.44 mmol, 1.10 equiv.) and Et₃N (532 mg, 5.25 mmol, 4.00 equiv.) in MeCN (10 mL) dropwise at 0° C. The resulting mixture was stirred overnight at room temperature and then diluted with water (50 mL). The resulting mixture was extracted with EtOAc (3×50 mL), washed with brine (2×20 mL), dried over anhydrous Na₂SO₄, filtered, and concentrated. The residue was purified by reversed-phase flash chromatography (C18 gel; mobile phase, MeCN in water (0.1% NH₃·H₂O), 0% to 100% gradient in 30 min; detector, UV 220 nm) to provide ethyl (3S)-3-[2-(5-bromo-2-fluorophenyl)-2-{5-[2-(3-fluoroazetidin-1-yl)ethyl]-3-methyl-2-oxopyridin-1-yl}acetamido]-3-{4-fluoro-2′-hydroxy-5,6′-dimethyl-[1,1′-biphenyl]-3-yl}propanoate (580 mg, 58.4%, mixture) as a white solid. LC-MS: (ES+H, m/z) [M+H]⁺=754.25.

### Step 2: Preparation of ethyl 2-[(14S,17S)-12,19-difluoro-14-{5-[2-(3-fluoroazetidin-1-yl)ethyl]-3-methyl-2-oxopyridin-1-yl}-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate

To a stirred mixture of ethyl (3S)-3-[2-(5-bromo-2-fluorophenyl)-2-{5-[2-(3-fluoroazetidin-1-yl)ethyl]-3-methyl-2-oxopyridin-1-yl}acetamido]-3-{4-fluoro-2′-hydroxy-5,6′-dimethyl-[1,1′-biphenyl]-3-yl}propanoate (150 mg, 0.20 mmol, 1.00 equiv.) in 1,4-dioxane (10 mL) was added K₂CO₃ (68 mg, 0.50 mmol, 2.50 equiv.), CuI (19 mg, 0.10 mmol, 0.50 equiv.) and (1R,2R)—N1,N2-dimethylcyclohexane-1,2-diamine (28 mg, 0.2 mmol, 1.00 equiv.) in portions at room temperature. The resulting mixture was stirred overnight at 100° C. under nitrogen atmosphere and then cooled to room temperature and diluted with water (50 mL). The reaction mixture was extracted with EtOAc (3×50 mL) and the combined organic layers were washed with brine (2×10 mL), dried over anhydrous Na₂SO₄, filtered, and concentrated. The residue was purified by reversed-phase flash chromatography (C18 gel; mobile phase, MeCN in water (0.1% NH₃·H₂O), 0% to 100% gradient in 30 min; detector, UV 220 nm) to afford ethyl 2-[(14S,17S)-12,19-difluoro-14-{5-[2-(3-fluoroazetidin-1-yl)ethyl]-3-methyl-2-oxopyridin-1-yl}-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (95 mg, 70.9%) as a yellow solid. LC-MS: (ES+H, m/z) [M+H]⁺=674.35. ¹H NMR (400 MHz, DMSO-d₆) δ 9.05 (d, J=7.5 Hz, 1H), 7.64-7.51 (m, 2H), 7.37 (t, J=7.8 Hz, 1H), 7.27-7.17 (m, 3H), 7.02 (d, J=6.2 Hz, 1H), 6.74-6.65 (m, 2H), 6.43 (dd, J=6.5, 2.2 Hz, 1H), 5.96 (dd, J=6.3, 3.0 Hz, 1H), 5.56-5.41 (m, 1H), 4.99 (dp, J=57.8, 5.1 Hz, 1H), 4.15-3.99 (m, 2H), 3.40-3.34 (m, 2H), 2.97-2.85 (m, 2H), 2.83-2.65 (m, 2H), 2.42 (td, J=7.2, 6.7, 2.3 Hz, 2H), 2.31-2.29 (m, 3H), 2.17 (t, J=7.0 Hz, 2H), 2.01 (s, 3H), 1.95 (s, 3H), 1.12 (t, J=7.1 Hz, 3H).

### Step 3: Preparation of [(14S,17S)-12,19-difluoro-14-{5-[2-(3-fluoroazetidin-1-yl)ethyl]-3-methyl-2-oxopyridin-1-yl}-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid

To a stirred mixture of ethyl 2-[(14S,17S)-12,19-difluoro-14-{5-[2-(3-fluoroazetidin-1-yl)ethyl]-3-methyl-2-oxopyridin-1-yl}-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (80 mg, 0.12 mmol, 1.00 equiv.) in THF (5 mL) and H₂O (1 mL) was added LiOH·H₂O (15 mg, 0.36 mmol, 3.00 equiv.) at room temperature under nitrogen atmosphere. The resulting mixture was stirred for 3 h at room temperature, and then acidified to pH 6 with 1 M HCl (aq.). The resulting mixture was concentrated under reduced pressure and the crude product was purified by Prep-HPLC (Column: XBridge Prep OBD C18 Column, 30*150 mm, 5 μm; Mobile Phase A: water (10 mmol/L NH₄HCO₃, Mobile Phase B: MeCN; Flow rate: 60 mL/min; Gradient: 20% B to 35% B in 10 min; Wavelength: 254 nm/220 nm; RT1 (min) 10.02) to afford [(14S,17S)-12,19-difluoro-14-{5-[2-(3-fluoroazetidin-1-yl)ethyl]-3-methyl-2-oxopyridin-1-yl}-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid (28.6 mg, 37.30%) as a white solid. LC-MS: (ES+H, m/z) [M+H]⁺=646.25. ¹H NMR (400 MHz, CD₃OD) δ 7.50 (dt, J=7.3, 3.6 Hz, 1H), 7.42 (d, J=8.0 Hz, 1H), 7.37-7.27 (m, 2H), 7.18-7.06 (m, 2H), 6.97-6.80 (m, 3H), 6.48 (dd, J=6.4, 2.2 Hz, 1H), 6.08 (dd, J=6.2, 3.0 Hz, 1H), 5.68 (t, J=7.4 Hz, 1H), 5.00-5.20 (m, 1H), 3.88-3.67 (m, 2H), 3.58-3.39 (m, 2H), 2.83 (t, J=7.3 Hz, 2H), 2.68 (qd, J=15.3, 7.4 Hz, 2H), 2.44-2.28 (m, 5H), 2.13 (s, 3H), 1.99 (s, 3H). ¹⁹F NMR (377 MHz, CD₃OD) δ −123.36, −126.74, −180.9.

### Examples 67 and 68

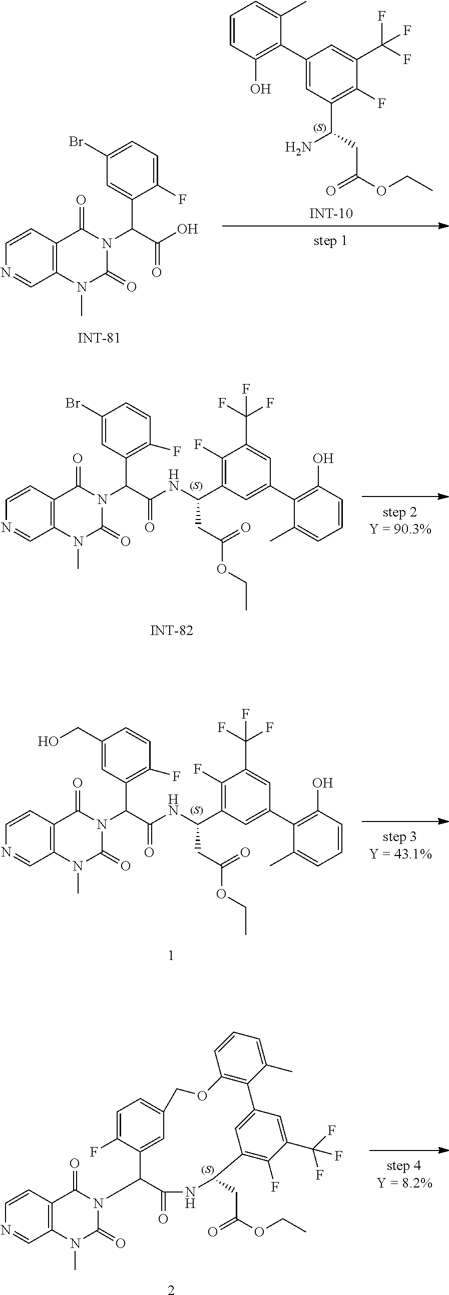

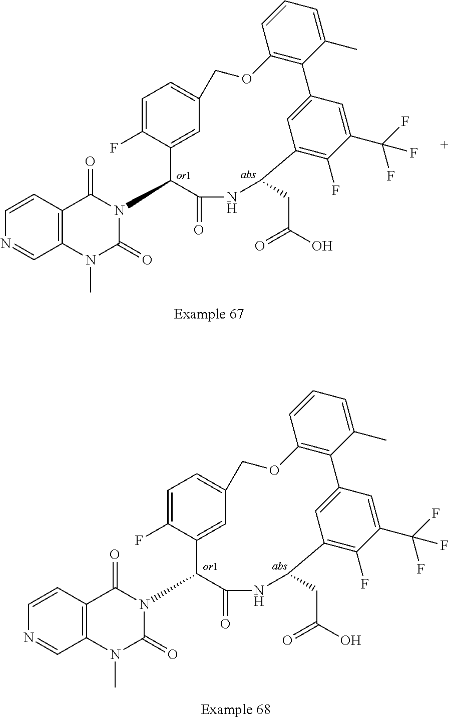 [SMILES: Cc1cccc2c1-c1cc(c(F)c(C(F)(F)F)c1)[C@H](CC(=O)O)NC(=O)[C@@H](n1c(=O)c3ccncc3n(C)c1=O)c1cc(ccc1F)CO2 ; "Example 67" ; Cc1cccc2c1-c1cc(c(F)c(C(F)(F)F)c1)[C@H](CC(=O)O)NC(=O)[C@H](n1c(=O)c3ccncc3n(C)c1=O)c1cc(ccc1F)CO2 ; "Example 68"]

### Step 1: Preparation of ethyl (3S)-3-[2-(5-bromo-2-fluorophenyl)-2-{1-methyl-2,4-dioxopyrido[3,4-d]pyrimidin-3-yl}acetamido]-3-[4-fluoro-2′-hydroxy-6′-methyl-5-(trifluoromethyl)-[1,1′-biphenyl]-3-yl]propanoate

To a stirred solution of (5-bromo-2-fluorophenyl)({1-methyl-2,4-dioxopyrido[3,4-d]pyrimidin-3-yl})acetic acid (250 mg, 0.61 mmol, 1.00 equiv.) and ethyl (3S)-3-amino-3-[4-fluoro-2′-hydroxy-6′-methyl-5-(trifluoromethyl)-[1,1′-biphenyl]-3-yl]propanoate (236 mg, 0.61 mmol, 1.00 equiv.) in MeCN (5 mL) was added TEA (247 mg, 2.44 mmol, 4.00 equiv.), HOBT (165 mg, 1.22 mmol, 2.00 equiv.) and EDCI (352 mg, 1.83 mmol, 3.00 equiv.) at room temperature under nitrogen atmosphere. The resulting mixture was stirred overnight at 40° C. under nitrogen atmosphere and then concentrated. The residue was purified by reversed-phase flash chromatography (C18 gel; mobile phase, MeCN in water (0.1% HCOOH), 45% to 80% gradient in 20 min; detector, UV 220 nm) to provide ethyl (3S)-3-[2-(5-bromo-2-fluorophenyl)-2-{1-methyl-2,4-dioxopyrido[3,4-d]pyrimidin-3-yl}acetamido]-3-[4-fluoro-2′-hydroxy-6′-methyl-5-(trifluoromethyl)-[1,1′-biphenyl]-3-yl]propanoate (180 mg, 37.9%) as light yellow solid. LC-MS: (ES+H, m/z) [M+H]⁺=775.0.

### Step 2: Preparation of ethyl (3S)-3-[4-fluoro-2′-hydroxy-6′-methyl-5-(trifluoromethyl)-[1,1′-biphenyl]-3-yl]-3-{2-[2-fluoro-5-(hydroxymethyl)phenyl]-2-{1-methyl-2,4-dioxopyrido[3,4-d]pyrimidin-3-yl}acetamido}propanoate

To a stirred solution of ethyl (3S)-3-[2-(5-bromo-2-fluorophenyl)-2-{1-methyl-2,4-dioxopyrido[3,4-d]pyrimidin-3-yl}acetamido]-3-[4-fluoro-2′-hydroxy-6′-methyl-5-(trifluoromethyl)-[1,1′-biphenyl]-3-yl]propanoate (160 mg, 0.20 mmol, 1.00 equiv.) in 1,4-dioxane (3 mL) was added (tributylstannyl)methanol (331 mg, 1.03 mmol, 5.00 equiv.) and 2nd Generation XPhos Precatalyst (16 mg, 0.02 mmol, 0.10 equiv.) at room temperature under nitrogen atmosphere. The resulting mixture was stirred overnight at 80° C. and then cooled to room temperature and concentrated. The residue was purified by reversed-phase flash chromatography (C18 gel; mobile phase, MeCN in water (0.1% NH₃·H₂O), 10% to 50% gradient in 10 min; detector, UV 254 nm) to provide ethyl (3S)-3-[4-fluoro-2′-hydroxy-6′-methyl-5-(trifluoromethyl)-[1,1′-biphenyl]-3-yl]-3-{2-[2-fluoro-5-(hydroxymethyl)phenyl]-2-{1-methyl-2,4-dioxopyrido[3,4-d]pyrimidin-3-yl}acetamido}propanoate (110 mg, 90.3%) as a yellow solid. LC-MS: (ES+H, m/z) [M+H]⁺=727.20).

### Step 3: Preparation of ethyl 2-[(15S,18S)-13,20-difluoro-3-methyl-15-{1-methyl-2,4-dioxopyrido[3,4-d]pyrimidin-3-yl}-16-oxo-21-(trifluoromethyl)-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetate

A solution of ethyl (3S)-3-[4-fluoro-2′-hydroxy-6′-methyl-5-(trifluoromethyl)-[1,1′-biphenyl]-3-yl]-3-{2-[2-fluoro-5-(hydroxymethyl)phenyl]-2-{1-methyl-2,4-dioxopyrido[3,4-d]pyrimidin-3-yl}acetamido}propanoate (95 mg, 0.13 mmol, 1.00 equiv.) and (tributylphosphoranylidene)acetonitrile (157 mg, 0.65 mmol, 5.00 equiv.) in toluene (5 mL) was stirred overnight at 100° C. under nitrogen atmosphere. The resulting mixture was then cooled to room temperature, concentrated and purified by reversed-phase flash chromatography (C18 gel; mobile phase, MeCN in water (0.1% NH₃·H₂O), 35% to 70% gradient in 20 min; detector, UV 220 nm) to provide ethyl 2-[(15S,18S)-13,20-difluoro-3-methyl-15-{1-methyl-2,4-dioxopyrido[3,4-d]pyrimidin-3-yl}-16-oxo-21-(trifluoromethyl)-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetate (40 mg, 43.18%, mixture) as a light yellow solid. LC-MS: (ES+H, m/z) [M+H]⁺=709.30. ¹H NMR (400 MHz, CD₃OD) δ ¹H NMR (400 MHz, CD₃OD) δ 8.83 (d, J=17.3 Hz, 1H), 8.51 (d, J=5.1 Hz, 1H), 8.10-7.89 (m, 1H), 7.75-56 (m, 1H), 7.44 (dd, J=18.2, 6.3 Hz, 1H), 7.32-7.24 (m, 1H), 7.14-6.88 (m, 4H), 5.94-5.67 (m, 1H), 5.26 (d, J=13.4 Hz, 1H), 5.11-4.93 (m, 1H), 4.14-4.01 (m, 2H), 3.71 (s, 1H), 3.62 (s, 1H), 3.52 (s, 1H), 3.08-2.88 (m, 2H), 2.11 (d, J=10.1 Hz, 3H), 1.21-1.10 (m, 3H).

### Step 4: Preparation of [(15S,18S)-13,20-difluoro-3-methyl-15-{1-methyl-2,4-dioxopyrido[3,4-d]pyrimidin-3-yl}-16-oxo-21-(trifluoromethyl)-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetic acid

To a stirred solution of ethyl 2-[(15S,18S)-13,20-difluoro-3-methyl-15-{1-methyl-2,4-dioxopyrido[3,4-d]pyrimidin-3-yl}-16-oxo-21-(trifluoromethyl)-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetate (35 mg, 0.049 mmol, 1.00 equiv.) in phosphate buffered saline (5 mL) was added Esterase from porcine liver (150 mg) at room temperature under nitrogen atmosphere. The resulting mixture was stirred for 9 days at 40° C. and then filtered. The filter cake was washed with MeOH (5×5 mL) and the combined filtrate was concentrated under reduced pressure. The crude product was purified by Prep-HPLC (Column: YMC-Actus Triart C18 ExRS Column, 30*150 mm, 5 μm; Mobile Phase A: water (10 mmol/L NH₄HCO₃+0.05% NH₃H₂O), Mobile Phase B: MeCN; Flow rate: 60 mL/min; Gradient: 20% B to 40% B in 10 min; Wavelength: 254 nm/220 nm) to afford [(15S,18S)-13,20-difluoro-3-methyl-15-{1-methyl-2,4-dioxopyrido[3,4-d]pyrimidin-3-yl}-16-oxo-21-(trifluoromethyl)-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetic acid (Example 67, 2.4 mg, 8.2%) and [(15R,18S)-13,20-difluoro-3-methyl-15-{1-methyl-2,4-dioxopyrido[3,4-d]pyrimidin-3-yl}-16-oxo-21-(trifluoromethyl)-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetic acid (Example 68, 0.3 mg, 0.8%) as a white solid. Example 67: LC-MS: (ES+H, m/z) [M+H]⁺=681.25. ¹H NMR (400 MHz, CD₃OD) δ 8.80 (s, 1H), 8.56-8.30 (m, 1H), 8.09-7.54 (m, 3H), 7.47-7.39 (m, 1H), 7.28 (t, J=7.9 Hz, 2H), 7.16-6.90 (m, 4H), 5.91 (s, 1H), 5.23 (d, J=13.2 Hz, 1H), 5.08 (d, J=12.7 Hz, 1H), 3.76-3.41 (m, 3H), 2.75 (s, 2H), 2.13 (s, 3H). Example 68: LC-MS: (ES+H, m/z) [M+H]⁺=681.20. ¹H NMR (400 MHz, CD₃OD) δ 8.84 (s, 1H), 8.50 (d, J=5.0 Hz, 1H), 7.98 (d, J=5.2 Hz, 2H), 7.66 (d, J=7.3 Hz, 1H), 7.38 (d, J=6.2 Hz, 1H), 7.31-7.19 (m, 2H), 7.04 (d, J=8.3 Hz, 1H), 6.99-6.91 (m, 2H), 6.88 (s, 1H), 5.66 (s, 1H), 5.28 (d, J=13.3 Hz, 1H), 4.97 (d, J=13.1 Hz, 1H), 3.61 (s, 3H), 2.79 (s, 2H), 2.09 (s, 3H).

### Example 69

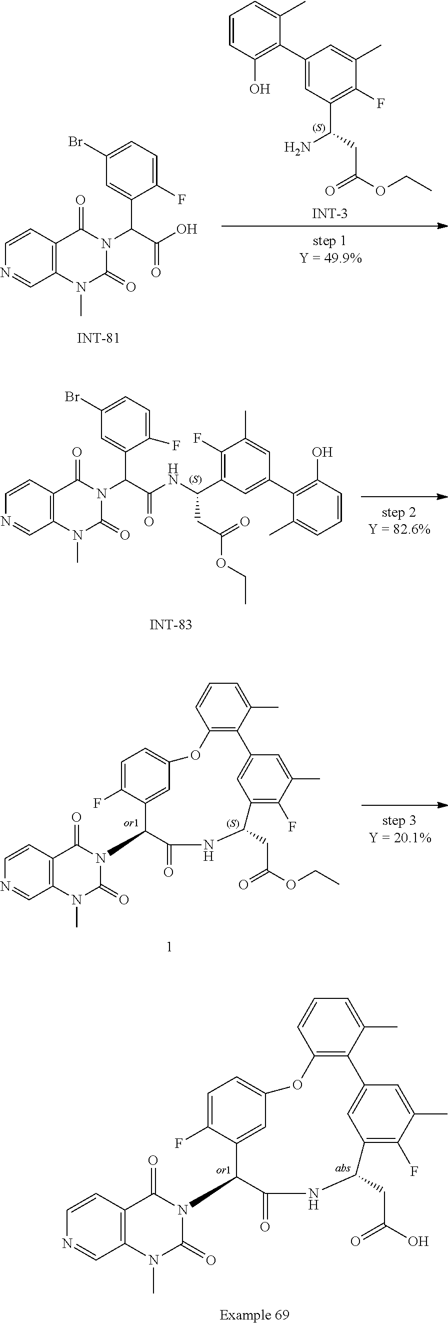

### Step 1: Preparation of ethyl (3S)-3-[2-(5-bromo-2-fluorophenyl)-2-{1-methyl-2,4-dioxopyrido[3,4-d]pyrimidin-3-yl}acetamido]-3-{4-fluoro-2′-hydroxy-5,6′-dimethyl-[1,1′-biphenyl]-3-yl}propanoate

To a stirred solution of (5-bromo-2-fluorophenyl)({1-methyl-2,4-dioxopyrido[3,4-d]pyrimidin-3-yl})acetic acid (170 mg, 0.41 mmol, 1.00 equiv.) and ethyl (3S)-3-amino-3-{4-fluoro-2′-hydroxy-5,6′-dimethyl-[1,1′-biphenyl]-3-yl}propanoate (165 mg, 0.49 mmol, 1.20 equiv.) in MeCN (3 mL) were added triethylamine (168 mg, 1.66 mmol, 4.00 equiv.), EDCI (239 mg, 1.24 mmol, 3.00 equiv.) and HOBT (112 mg, 0.83 mmol, 2.00 equiv.) at room temperature under nitrogen atmosphere. The resulting mixture was stirred overnight at 40° C. and then diluted with water (30 mL) and extracted with EtOAc (3×20 mL). The combined organic layers were washed with brine (1×5 mL), dried over anhydrous Na₂SO₄, filtered, and concentrated. The residue was purified by silica gel column chromatography (petroleum ether/EtOAc (50% to 90% gradient in 20 min) to afford ethyl (3S)-3-[2-(5-bromo-2-fluorophenyl)-2-{1-methyl-2,4-dioxopyrido[3,4-d]pyrimidin-3-yl}acetamido]-3-{4-fluoro-2′-hydroxy-5,6′-dimethyl-[1,1′-biphenyl]-3-yl}propanoate (150 mg, 49.9%) as light yellow solid. LC-MS: (ES+H, m/z) [M+H]⁺=720.9.

### Step 2: Preparation of ethyl 2-[(14S,17S)-12,19-difluoro-3,20-dimethyl-14-{1-methyl-2,4-dioxopyrido[3,4-d]pyrimidin-3-yl}-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate

To a stirred solution of ethyl (3S)-3-[2-(5-bromo-2-fluorophenyl)-2-{1-methyl-2,4-dioxopyrido[3,4-d]pyrimidin-3-yl}acetamido]-3-{4-fluoro-2′-hydroxy-5,6′-dimethyl-[1,1′-biphenyl]-3-yl}propanoate (150 mg, 0.20 mmol, 1.00 equiv.) in 1,4-dioxane (4 mL) was added CuI (19 mg, 0.10 mmol, 0.50 equiv.), K₂CO₃ (43 mg, 0.31 mmol, 1.50 equiv.) and (1R,2R)—N1,N2-dimethylcyclohexane-1,2-diamine (29 mg, 0.20 mmol, 1.00 equiv.) at room temperature under nitrogen atmosphere. The reaction mixture was stirred for 2 h at 100° C. and then cooled to room temperature and concentrated. The residue was purified by reversed-phase flash chromatography (C18 gel; mobile phase, MeCN in water (0.1% NH₃·H₂O), 30% to 60% gradient in 60 min; detector, UV 254 nm) to provide ethyl 2-[(14S,17S)-12,19-difluoro-3,20-dimethyl-14-{1-methyl-2,4-dioxopyrido[3,4-d]pyrimidin-3-yl}-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (110 mg, 82.6%) as a light yellow solid. LC-MS: (ES+H, m/z) [M+H]⁺=641.30. ¹H NMR (400 MHz, CD₃OD) δ 8.81 (s, 1H), 8.64-8.31 (m, 1H), 7.49-7.28 (m, 3H), 7.15 (d, J=7.6 Hz, 1H), 7.07-6.91 (m, 3H), 6.57 (dd, J=6.4, 2.2 Hz, 1H), 6.39 (dd, J=6.5, 3.0 Hz, 1H), 5.89 (t, J=7.8 Hz, 1H), 4.18-4.05 (m, 2H), 3.74-3.44 (m, 3H), 2.89-2.70 (m, 2H), 2.37 (d, J=2.0 Hz, 3H), 2.03 (s, 4H), 1.19 (t, J=7.1 Hz, 3H).

### Step 3: Preparation of [(14S,17S)-12,19-difluoro-3,20-dimethyl-14-{1-methyl-2,4-dioxopyrido[3,4-d]pyrimidin-3-yl}-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid

To a stirred solution of ethyl 2-[(14S,17S)-12,19-difluoro-3,20-dimethyl-14-{1-methyl-2,4-dioxopyrido[3,4-d]pyrimidin-3-yl}-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (95 mg, 0.14 mmol, 1.00 equiv.) in phosphate buffered saline (10 mL) was added Esterase from porcine liver (300 mg) at room temperature under nitrogen atmosphere. The reaction mixture was stirred for 4 days at 40° C. and then filtered. The filter cake was washed with MeOH (5×5 mL) and the combined filtrate was concentrated under reduced pressure. The crude product (100 mg) was purified by Prep-HPLC (Column: YMC-Actus Triart C18 ExRS Column, 30*150 mm, 5 μm; Mobile Phase A: water (10 mmol/L NH₄HCO₃+0.05% NH₃·H₂O), Mobile Phase B: MeCN; Flow rate: 60 mL/min; Gradient: 20% B to 40% B in 10 min; Wavelength: 254 nm/220 nm; RT1 (min) 9.6/10.5) to afford [(14S,17S)-12,19-difluoro-3,20-dimethyl-14-{1-methyl-2,4-dioxopyrido[3,4-d]pyrimidin-3-yl}-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid (18.3 mg, 20.1%) as a white solid. LC-MS: (ES+H, m/z) [M+H]⁺=613.20. ¹H NMR (300 MHz, DMSO-d₆) δ 8.92 (d, J=7.3 Hz, 1H), 8.80 (s, 1H), 8.53 (dd, J=15.3, 5.1 Hz, 1H), 7.82 (dd, J=78.6, 5.0 Hz, 1H), 7.58 (d, J=8.0 Hz, 1H), 7.51-7.27 (m, 2H), 7.19 (d, J=7.6 Hz, 1H), 7.12-6.98 (m, 2H), 6.81-6.67 (m, 1H), 6.63-6.57 (m, 1H) 6.24 (d, J=7.2 Hz, 1H), 5.64 (d, J=8.1 Hz, 1H), 3.69-3.41 (m, 3H), 2.59 (t, J=6.7 Hz, 2H), 2.32 (d, J=1.8 Hz, 3H), 1.99 (s, 3H).

### Example 70

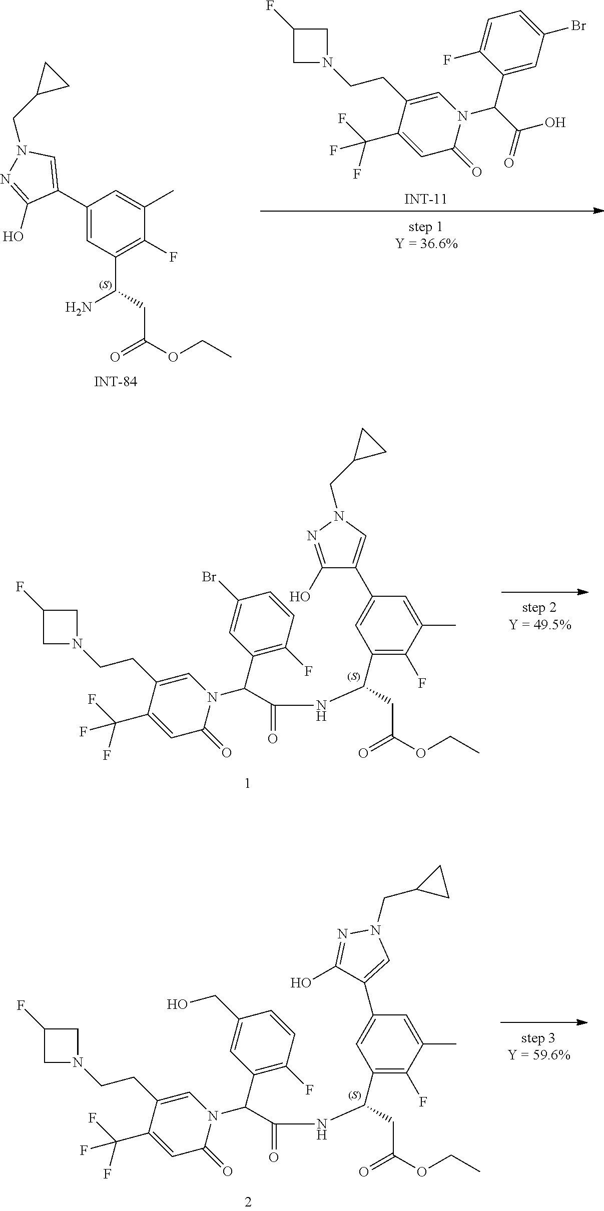

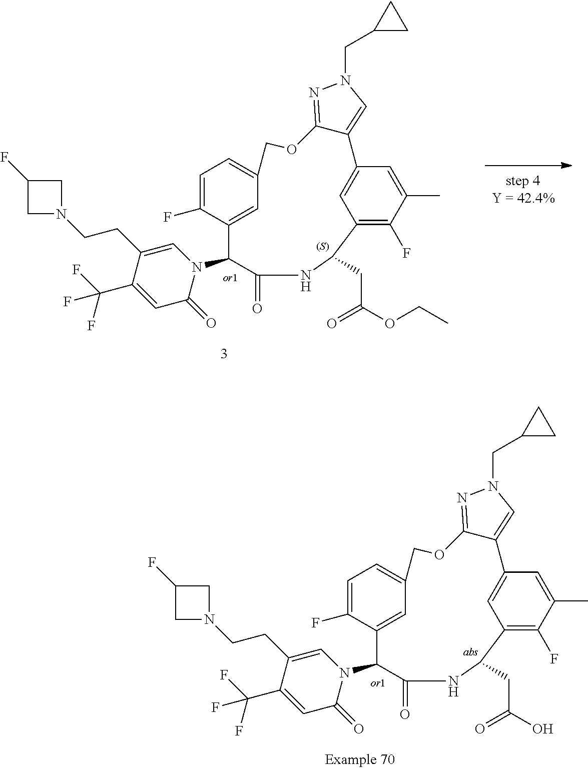

### Step 1: Preparation of ethyl (3S)-3-[2-(5-bromo-2-fluorophenyl)-2-{5-[2-(3-fluoroazetidin-1-yl)ethyl]-2-oxo-4-(trifluoromethyl)pyridin-1-yl}acetamido]-3-{5-[1-(cyclopropylmethyl)-3-hydroxypyrazol-4-yl]-2-fluoro-3-methylphenyl}propanoate

To a stirred solution of ethyl (3S)-3-amino-3-{5-[1-(cyclopropylmethyl)-3-hydroxypyrazol-4-yl]-2-fluoro-3-methylphenyl}propanoate (200 mg, 0.55 mmol, 1.00 equiv.) and (5-bromo-2-fluorophenyl)({5-[2-(3-fluoroazetidin-1-yl)ethyl]-2-oxo-4-(trifluoromethyl)pyridin-1-yl})acetic acid (274 mg, 0.55 mmol, 1.00 equiv.) in MeCN (5 mL) was added triethylamine (112 mg, 1.10 mmol, 2.00 equiv.) at room temperature. The resulting mixture was stirred for 10 min at room temperature under nitrogen atmosphere and then EDCI (318 mg, 1.65 mmol, 3.00 equiv.) and HOBT (149 mg, 1.10 mmol, 2.00 equiv.) were added. The reaction mixture was stirred overnight at 50° C. and then cooled to room temperature and diluted with water (20 mL). The reaction mixture was extracted with EtOAc (3×30 mL) and the combined organic layers were washed with brine (2×20 mL), dried over anhydrous Na₂SO₄, filtered, and concentrated. The residue was purified by silica gel column chromatography (petroleum ether/EtOAc (20% to 60%) to afford ethyl (3S)-3-[2-(5-bromo-2-fluorophenyl)-2-{5-[2-(3-fluoroazetidin-1-yl)ethyl]-2-oxo-4-(trifluoromethyl)pyridin-1-yl}acetamido]-3-{5-[1-(cyclopropylmethyl)-3-hydroxypyrazol-4-yl]-2-fluoro-3-methylphenyl}propanoate (170 mg, 36.6%) as a yellow oil. LC-MS: (ES+H, m/z) [M+H]⁺=839.85. ¹H NMR (400 MHz, DMSO-d₆) δ 10.29 (s, 1H), 9.41 (dd, J=36.3, 7.9 Hz, 1H), 7.93 (d, J=4.3 Hz, 1H), 7.83-7.73 (m, 1H), 7.73-7.55 (m, 1H), 7.53-7.47 (m, 1H), 7.44-7.24 (m, 3H), 6.90-6.71 (m, 2H), 5.56-5.41 (m, 1H), 5.07-4.77 (m, 1H), 3.83-3.71 (m, 2H), 3.30 (d, J=6.2 Hz, 2H), 2.97-2.70 (m, 4H), 2.66-2.55 (m, 1H), 2.45 (d, J=5.0 Hz, 2H), 2.33 (d, J=24.6 Hz, 3H), 2.23 (dd, J=10.0, 1.8 Hz, 3H), 1.13-1.06 (m, 3H), 1.02 (t, J=7.5 Hz, 1H), 0.60-0.48 (m, 2H), 0.40-0.29 (m, 2H).

### Step 2: Preparation of ethyl (3S)-3-{5-[1-(cyclopropylmethyl)-3-hydroxypyrazol-4-yl]-2-fluoro-3-methylphenyl}-3-{2-[2-fluoro-5-(hydroxymethyl)phenyl]-2-{5-[2-(3-fluoroazetidin-1-yl)ethyl]-2-oxo-4-(trifluoromethyl)pyridin-1-yl}acetamido}propanoate

To a stirred solution of ethyl (3S)-3-[2-(5-bromo-2-fluorophenyl)-2-{5-[2-(3-fluoroazetidin-1-yl)ethyl]-2-oxo-4-(trifluoromethyl)pyridin-1-yl}acetamido]-3-{5-[1-(cyclopropylmethyl)-3-hydroxypyrazol-4-yl]-2-fluoro-3-methylphenyl}propanoate (150 mg, 0.18 mmol, 1.00 equiv.) in 1,4-dioxane (5 mL) were added (tributylstannyl)methanol (229 mg, 0.71 mmol, 4.00 equiv.) and 2nd generation XPhos Precatalyst (14 mg, 0.01 mmol, 0.10 equiv.) at room temperature. The reaction mixture was stirred for 3 days at 80° C. under nitrogen atmosphere and then cooled to room temperature and diluted with water (20 mL). The resulting mixture was extracted with EtOAc (3×20 mL) and the combined organic layers were washed with brine (2×20 mL), dried over anhydrous Na₂SO₄, filtered, and concentrated. The residue was purified by reversed-phase flash chromatography (C18 XBridge; mobile phase, MeCN in water (0.1% NH₃·H₂O), 10% to 50% gradient in 10 min; detector, UV 220 nm) to afford ethyl (3S)-3-{5-[1-(cyclopropylmethyl)-3-hydroxypyrazol-4-yl]-2-fluoro-3-methylphenyl}-3-{2-[2-fluoro-5-(hydroxymethyl)phenyl]-2-{5-[2-(3-fluoroazetidin-1-yl)ethyl]-2-oxo-4-(trifluoromethyl)pyridin-1-yl}acetamido}propanoate (70 mg, 49.5%, mixture) as yellow oil. LC-MS: (ES+H, m/z) [M+H]⁺=790.30.

### Step 3: Preparation of ethyl 2-[(14S,17S)-4-(cyclopropylmethyl)-12,19-difluoro-14-{5-[2-(3-fluoroazetidin-1-yl)ethyl]-2-oxo-4-(trifluoromethyl)pyridin-1-yl}-20-methyl-15-oxo-7-oxa-4,5,16-triazatetracyclo[16.3.1.1\hat{ }{9,13}.0\hat{ }{2,6}]tricosa-1(22),2,5,9,11,13(23),18,20-octaen-17-yl]acetate

To a stirred solution of ethyl (3S)-3-{5-[1-(cyclopropylmethyl)-3-hydroxypyrazol-4-yl]-2-fluoro-3-methylphenyl}-3-{2-[2-fluoro-5-(hydroxymethyl)phenyl]-2-{5-[2-(3-fluoroazetidin-1-yl)ethyl]-2-oxo-4-(trifluoromethyl)pyridin-1-yl}acetamido}propanoate (60 mg, 0.08 mmol, 1.00 equiv.) in toluene (3 mL) was added 2-(tributyl-1\hat{ }[5]-phosphanylidene)acetonitrile (92 mg, 0.38 mmol, 5.00 equiv.) at room temperature under nitrogen atmosphere. The resulting mixture was stirred overnight at 100° C. and then cooled to room temperature and concentrated. The residue was purified by silica gel column chromatography (CH₂Cl₂/MeOH (0% to 10% gradient in 10 min) to afford ethyl 2-[(14S,17S)-4-(cyclopropylmethyl)-12,19-difluoro-14-{5-[2-(3-fluoroazetidin-1-yl)ethyl]-2-oxo-4-(trifluoromethyl)pyridin-1-yl}-20-methyl-15-oxo-7-oxa-4,5,16-triazatetracyclo[16.3.1.1\hat{ }{9,13}.0\hat{ }{2,6}]tricosa-1(22),2,5,9,11,13(23),18,20-octaen-17-yl]acetate (35 mg, 59.6%) as a yellow oil. LC-MS: (ES+H, m/z) [M+H]⁺=772.35.

### Step 4: Preparation of [(14S,17S)-4-(cyclopropylmethyl)-12,19-difluoro-14-{5-[2-(3-fluoroazetidin-1-yl)ethyl]-2-oxo-4-(trifluoromethyl)pyridin-1-yl}-20-methyl-15-oxo-7-oxa-4,5,16-triazatetracyclo[16.3.1.1\hat{ }{9,13}.0\hat{ }{2,6}]tricosa-1(22),2,5,9,11,13(23),18,20-octaen-17-yl]acetic acid

To a stirred solution of ethyl 2-[(14S,17S)-4-(cyclopropylmethyl)-12,19-difluoro-14-{5-[2-(3-fluoroazetidin-1-yl)ethyl]-2-oxo-4-(trifluoromethyl)pyridin-1-yl}-20-methyl-15-oxo-7-oxa-4,5,16-triazatetracyclo[16.3.1.1\hat{ }{9,13}.0\hat{ }{2,6}]tricosa-1(22),2,5,9,11,13(23),18,20-octaen-17-yl]acetate (25 mg, 0.03 mmol, 1.00 equiv.) in THF/H₂O=(5 mL/1 mL) was added LiOH·H₂O (3 mg, 0.12 mmol, 4.00 equiv.) at room temperature. The reaction mixture was stirred for 2 h at room temperature under nitrogen atmosphere and then acidified to pH 5 with 1 M HCl (aq.) and concentrated. The crude product was purified by reversed-phase flash chromatography (Column: XBridge Shield RP18 OBD Column 30*150 mm, 5 μm; Mobile Phase A: water (0.1% HCOOH), Mobile Phase B: MeCN; Flow rate: 60 mL/min; Gradient: 24% B to 46% B in 8 min; Wavelength: 254 nm/220 nm) to provide [(14S,17S)-4-(cyclopropylmethyl)-12,19-difluoro-14-{5-[2-(3-fluoroazetidin-1-yl)ethyl]-2-oxo-4-(trifluoromethyl)pyridin-1-yl}-20-methyl-15-oxo-7-oxa-4,5,16-triazatetracyclo[16.3.1.1\hat{ }{9,13}.0\hat{ }{2,6}]tricosa-1(22),2,5,9,11,13(23),18,20-octaen-17-yl]acetic acid (10.7 mg, 42.4%) as white solid. LC-MS: (ES+H, m/z) [M+H]⁺=744.25. ¹H NMR (400 MHz, DMSO-d₆) δ 9.27-9.15 (m, 1H), 8.03 (s, 1H), 7.91-7.82 (m, 1H), 7.70 (dd, J=7.0, 2.3 Hz, 1H), 7.65-7.55 (m, 1H), 7.36-7.25 (m, 2H), 7.19 (s, 1H), 7.05 (s, 1H), 6.80 (s, 1H), 5.46-5.40 (m, 1H), 5.37 (d, J=12.5 Hz, 1H), 5.15 (d, J=12.3 Hz, 1H), 4.92-4.70 (m, 1H), 3.86 (d, J=7.1 Hz, 2H), 3.33-3.14 (m, 3H), 2.90-2.74 (m, 2H), 2.62-2.55 (m, 1H), 2.44-2.17 (m, 7H), 1.32-1.22 (m, 1H), 0.61-0.49 (m, 2H), 0.44-0.32 (m, 2H). ¹⁹F NMR (377 MHz, DMSO-d₆) δ −62.55, −117.69, −127.89, −176.96, −177.25.

### Example 71

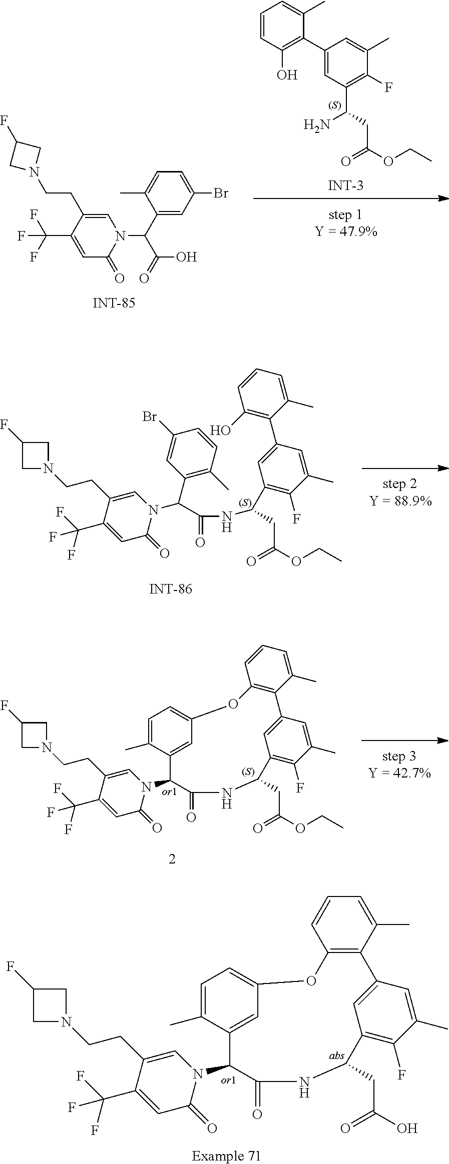

### Step 1: Preparation of ethyl (3S)-3-(2-(5-bromo-2-methylphenyl)-2-(5-(2-(3-fluoroazetidin-1-yl)ethyl)-2-oxo-4-(trifluoromethyl)pyridin-1(2H)-yl)acetamido)-3-(4-fluoro-2′-hydroxy-5,6′-dimethyl-[1,1′-biphenyl]-3-yl)propanoate

A mixture of (5-bromo-2-methylphenyl)({5-[2-(3-fluoroazetidin-1-yl)ethyl]-2-oxo-4-(trifluoromethyl)pyridin-1-yl})acetic acid; trifluoroacetaldehyde (550 mg, 0.93 mmol, 1.00 equiv.), HOBT (163 mg, 1.21 mmol, 1.30 equiv.), EDCI (268 mg, 1.40 mmol, 1.50 equiv.) and DIEA (603 mg, 4.66 mmol, 5.00 equiv.) in MeCN (5 mL) was stirred for 40 min at 40° C. under nitrogen atmosphere. To the above mixture was added ethyl (3S)-3-amino-3-{4-fluoro-2′-hydroxy-5,6′-dimethyl-[1,1′-biphenyl]-3-yl}propanoate hydrochloride (343 mg, 0.93 mmol, 1.00 equiv.) in portions at room temperature. The resulting mixture was stirred overnight at 40° C. and then cooled to room temperature. The residue was purified by reversed-phase flash chromatography (C18 gel; mobile phase, MeCN in water (0.1% NH₃·H₂O), 50% to 70% gradient in 10 min; detector, UV 254 nm) to provide ethyl (3S)-3-(2-(5-bromo-2-methylphenyl)-2-(5-(2-(3-fluoroazetidin-1-yl)ethyl)-2-oxo-4-(trifluoromethyl)pyridin-1(2H)-yl)acetamido)-3-(4-fluoro-2′-hydroxy-5,6′-dimethyl-[1,1′-biphenyl]-3-yl)propanoate (360 mg, 47.9%, mixture) as an off-white solid. LC-MS: (ES+H, m/z) [M+H]⁺=804.3.

### Step 2: Preparation of ethyl 2-((5S,8S)-14-fluoro-5-(5-(2-(3-fluoroazetidin-1-yl)ethyl)-2-oxo-4-(trifluoromethyl)pyridin-1(2H)-yl)-15,26,44-trimethyl-6-oxo-3-oxa-7-aza-1,4(1,3),2(1,2)-tribenzenacyclooctaphane-8-yl)acetate

A mixture of ethyl (3S)-3-[2-(5-bromo-2-methylphenyl)-2-{5-[2-(3-fluoroazetidin-1-yl)ethyl]-2-oxo-4-(trifluoromethyl)pyridin-1-yl}acetamido]-3-{4-fluoro-2′-hydroxy-5,6′-dimethyl-[1,1′-biphenyl]-3-yl}propanoate (100 mg, 0.12 mmol, 1.00 equiv.), CuI (11 mg, 0.06 mmol, 0.50 equiv.), K₂CO₃ (34 mg, 0.24 mmol, 2.00 equiv.), and (1R,2R)—N1,N2-dimethylcyclohexane-1,2-diamine (17 mg, 0.12 mmol, 1.00 equiv.) in 1,4-dioxane was stirred overnight at 100° C. under nitrogen atmosphere. The mixture was then cooled to room temperature and concentrated. The residue was purified by reversed-phase flash chromatography (C18 gel; mobile phase, MeCN in water (0.1% NH₃·H₂O), 50% to 70% gradient in 10 min; detector, UV 254 nm) to provide ethyl 2-((5S,8S)-14-fluoro-5-(5-(2-(3-fluoroazetidin-1-yl)ethyl)-2-oxo-4-(trifluoromethyl)pyridin-1(2H)-yl)-15,26,44-trimethyl-6-oxo-3-oxa-7-aza-1,4(1,3),2(1,2)-tribenzenacyclooctaphane-8-yl)acetate (80 mg, 88.9%) as an off-white solid. LC-MS: (ES+H, m/z) [M+H]⁺=724.3.

### Step 3: Preparation of 2-((5S,8S)-14-fluoro-5-(5-(2-(3-fluoroazetidin-1-yl)ethyl)-2-oxo-4-(trifluoromethyl)pyridin-1(2H)-yl)-15,26,44-trimethyl-6-oxo-3-oxa-7-aza-1,4(1,3),2(1,2)-tribenzenacyclooctaphane-8-yl)acetic acid

A mixture of ethyl 2-[(14S,17S)-19-fluoro-14-{5-[2-(3-fluoroazetidin-1-yl)ethyl]-2-oxo-4-(trifluoromethyl)pyridin-1-yl}-3,12,20-trimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (80 mg, 0.11 mmol, 1.00 equiv.) and LiOH·H₂O (13 mg, 0.33 mmol, 3.00 equiv.) in THF/H₂O=5/1 (6 mL) was stirred for 1 h at room temperature under nitrogen atmosphere and then concentrated. The crude product was purified by Prep-HPLC (Column: Xselect CSH C18 OBD Column 30*150 mm, 5 μm; Mobile Phase A: water (0.1% HCOOH), Mobile Phase B: MeCN; Flow rate: 60 mL/min; Gradient: 16% B to 35% B in 8 min; Wavelength: 254 nm/220 nm) to provide 2-((5S,8S)-14-fluoro-5-(5-(2-(3-fluoroazetidin-1-yl)ethyl)-2-oxo-4-(trifluoromethyl)pyridin-1(2H)-yl)-15,26,44-trimethyl-6-oxo-3-oxa-7-aza-1,4(1,3),2(1,2)-tribenzenacyclooctaphane-8-yl)acetic acid (33.4 mg, 42.7%) as a white solid. LC-MS: (ES+H, m/z) [M+H]⁺=696.30. ¹H NMR (300 MHz, DMSO-d₆) δ 13.1-11.2 (m, 1H), 9.06 (s, 1H), 7.60 (d, J=8.0 Hz, 1H), 7.51 (dd, J=8.4, 2.6 Hz, 1H), 7.35 (t, J=7.9 Hz, 1H), 7.26 (d, J=8.5 Hz, 1H), 7.17 (d, J=7.6 Hz, 1H), 7.06 (s, 1H), 7.01 (dd, J=6.9, 2.1 Hz, 1H), 6.80 (s, 1H), 6.58-6.46 (m, 2H), 6.01 (d, J=2.5 Hz, 1H), 5.49 (q, J=7.3 Hz, 1H), 4.83 (dt, J=57.8, 5.3 Hz, 1H), 3.24-3.07 (m, 2H), 2.86 (t, J=4.1 Hz, 1H), 2.77 (s, 1H), 2.65 (t, J=7.3 Hz, 2H), 2.41-2.23 (m, 7H), 1.98 (d, J=9.9 Hz, 6H). ¹⁹F NMR (282 MHz, DMSO-d₆) δ −62.54, −126.13, −176.78.

### Example 72

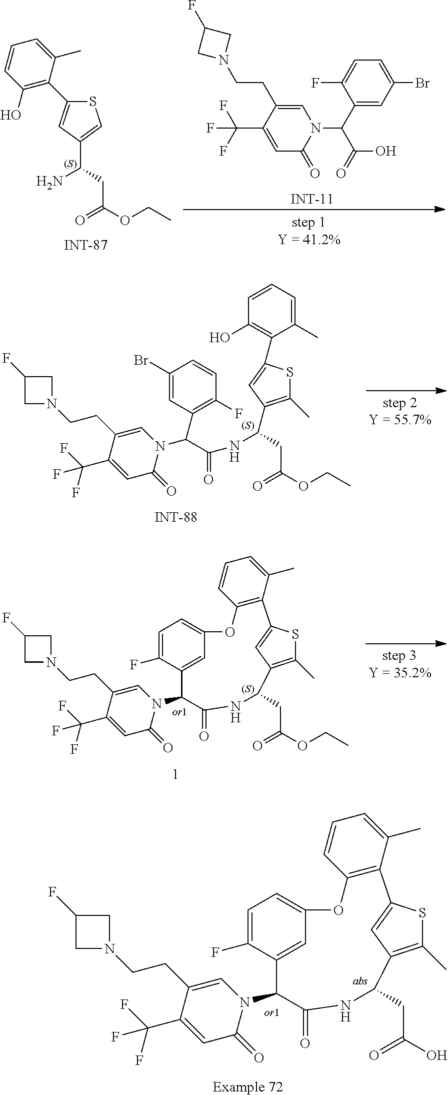

### Step 1: Preparation of ethyl (3S)-3-[2-(5-bromo-2-fluorophenyl)-2-{5-[2-(3-fluoroazetidin-1-yl)ethyl]-2-oxo-4-(trifluoromethyl)pyridin-1-yl}acetamido]-3-[5-(2-hydroxy-6-methylphenyl)-2-methylthiophen-3-yl]propanoate

A mixture of (5-bromo-2-fluorophenyl)({5-[2-(3-fluoroazetidin-1-yl)ethyl]-2-oxo-4-(trifluoromethyl)pyridin-1-yl})acetic acid (602 mg, 1.22 mmol, 1.05 equiv.), HOBT (313 mg, 2.32 mmol, 2.00 equiv.) and EDCI (444 mg, 2.32 mmol, 2.00 equiv.) in MeCN (15 mL) was stirred for 1 h at 30° C. under nitrogen atmosphere. To the above mixture was added ethyl (3S)-3-amino-3-[5-(2-hydroxy-6-methylphenyl)-2-methylthiophen-3-yl]propanoate (370 mg, 1.16 mmol, 1.00 equiv.) in MeCN (5 mL) dropwise at room temperature. The resulting mixture was stirred for 1 day at 30° C. and then cooled to room temperature and concentrated. The residue was purified by reversed-phase flash chromatography (C18 gel; mobile phase, MeCN in water (0.1% NH₃·H₂O), 40% to 80% gradient in 20 min; detector, UV 220 nm) to provide ethyl (3S)-3-[2-(5-bromo-2-fluorophenyl)-2-{5-[2-(3-fluoroazetidin-1-yl)ethyl]-2-oxo-4-(trifluoromethyl)pyridin-1-yl}acetamido]-3-[5-(2-hydroxy-6-methylphenyl)-2-methylthiophen-3-yl]propanoate (380 mg, 41.2%, mixture) as light yellow solid. LC-MS: (ES+H, m/z) [M+H]⁺=786.10.

### Step 2: Preparation of ethyl 2-[(13S,16S)-18-fluoro-16-{5-[2-(3-fluoroazetidin-1-yl)ethyl]-2-oxo-4-(trifluoromethyl)pyridin-1-yl}-7,11-dimethyl-15-oxo-2-oxa-10-thia-14-azatetracyclo[15.3.1.1\hat{ }{9,12}.0\hat{ }{3,8}]docosa-1(20),3(8),4,6,9(22),11,17(21),18-octaen-13-yl]acetate

A mixture of ethyl (3S)-3-[2-(5-bromo-2-fluorophenyl)-2-{5-[2-(3-fluoroazetidin-1-yl)ethyl]-2-oxo-4-(trifluoromethyl)pyridin-1-yl}acetamido]-3-[5-(2-hydroxy-6-methylphenyl)-2-methylthiophen-3-yl]propanoate (100 mg, 0.13 mmol, 1.00 equiv.), CuI (12 mg, 0.06 mmol, 0.50 equiv.), K₂CO₃ (35 mg, 0.25 mmol, 2.00 equiv.) and (1R,2R)—N1,N2-dimethylcyclohexane-1,2-diamine (18 mg, 0.13 mmol, 1.00 equiv.) in dioxane (5 mL) was stirred overnight at 100° C. under nitrogen atmosphere and then cooled to room temperature. The resulting mixture was diluted with water (10 mL) and extracted with EtOAc (3×20 mL). The combined organic layers were washed with brine (3×10 mL), dried over anhydrous Na₂SO₄, filtered, and concentrated. The residue was purified by reversed-phase flash chromatography (C18 gel; mobile phase, MeCN in water (0.1% NH₃·H₂O), 40% to 80% gradient in 20 min; detector, UV 220 nm) to provide ethyl 2-[(13S,16S)-18-fluoro-16-{5-[2-(3-fluoroazetidin-1-yl)ethyl]-2-oxo-4-(trifluoromethyl)pyridin-1-yl}-7,11-dimethyl-15-oxo-2-oxa-10-thia-14-azatetracyclo[15.3.1.1\hat{ }{9,12}.0\hat{ }{3,8}]docosa-1(20),3(8),4,6,9(22),11,17(21),18-octaen-13-yl]acetate (50 mg, 55.7%) as a white solid. LC-MS: (ES+H, m/z) [M+H]⁺=716.30. ¹H NMR (400 MHz, DMSO-d₆) δ 8.95 (d, J=8.0 Hz, 1H), 7.52 (d, J=7.9 Hz, 1H), 7.42 (t, J=7.8 Hz, 1H), 7.39-7.33 (m, 1H), 7.29 (s, 1H), 7.25-7.21 (m, 2H), 6.81 (s, 1H), 6.59 (s, 1H), 6.00 (s, 1H), 5.77 (dd, J=6.2, 3.0 Hz, 1H), 5.12 (q, J=7.9 Hz, 1H), 5.04-4.82 (m, 1H), 4.12-4.02 (m, 2H), 3.33-3.25 (m, 3H), 2.94-2.81 (m, 3H), 2.72 (dd, J=15.6, 7.8 Hz, 2H), 2.46-2.31 (m, 5H), 2.12 (s, 3H), 1.13 (t, J=7.1 Hz, 3H). ¹⁹F NMR (377 MHz, DMSO-d₆) δ −62.61, −121.72, −176.96.

### Step 3: Preparation of [(13S,16S)-18-fluoro-16-{5-[2-(3-fluoroazetidin-1-yl)ethyl]-2-oxo-4-(trifluoromethyl)pyridin-1-yl}-7,11-dimethyl-15-oxo-2-oxa-10-thia-14-azatetracyclo[15.3.1.1\hat{ }{9,12}.0\hat{ }{3,8}]docosa-1(20),3(8),4,6,9(22),11,17(21),18-octaen-13-yl]acetic acid

A mixture of ethyl 2-[(13S,16S)-18-fluoro-16-{5-[2-(3-fluoroazetidin-1-yl)ethyl]-2-oxo-4-(trifluoromethyl)pyridin-1-yl}-7,11-dimethyl-15-oxo-2-oxa-10-thia-14-azatetracyclo[15.3.1.1\hat{ }{9,12}.0\hat{ }{3,8}]docosa-1(20),3(8),4,6,9(22),11,17(21),18-octaen-13-yl]acetate (40 mg, 0.06 mmol, 1.00 equiv.) and LiOH·H₂O (10 mg, 0.22 mmol, 4.00 equiv.) in THF (4 mL) and H₂O (1 mL) was stirred overnight at room temperature under nitrogen atmosphere. The mixture was then acidified to pH 5 with HCl (aq. 1 M) and concentrated. The crude product was purified by Prep-HPLC (Column: XBridge Prep OBD C18 Column, 30*150 mm, 10 μm; Mobile Phase A: water (10 mmol/L NH₄HCO₃+0.05% NH₃H₂O), Mobile Phase B: MeCN; Flow rate: 60 mL/min; Gradient: 29% B to 45% B in 8 min; Wavelength: 254 nm/220 nm; RT1 (min) 8.72) to afford [(13S,16S)-18-fluoro-16-{5-[2-(3-fluoroazetidin-1-yl)ethyl]-2-oxo-4-(trifluoromethyl)pyridin-1-yl}-7,11-dimethyl-15-oxo-2-oxa-10-thia-14-azatetracyclo[15.3.1.1\hat{ }{9,12}.0\hat{ }{3,8}]docosa-1(20),3(8),4,6,9(22),11,17(21),18-octaen-13-yl]acetic acid (14.1 mg, 35.2%) as a white solid. LC-MS: (ES+H, m/z) [M+H]⁺=688.10. ¹H NMR (400 MHz, CD₃OD) δ 7.45-7.28 (m, 4H), 7.20-7.05 (m, 2H), 6.87 (s, 1H), 6.76 (s, 1H), 6.05-5.92 (m, 2H), 5.32 (dd, J=8.5, 6.8 Hz, 1H), 5.11-4.95 (m, 1H), 3.66-3.47 (m, 2H), 3.26-3.11 (m, 2H), 2.82-2.65 (m, 3H), 2.60 (s, 4H), 2.55-2.41 (m, 2H), 2.16 (s, 3H). ¹⁹F NMR (377 MHz, CD₃OD) δ −64.98, −123.48, −180.37.

### Example 73

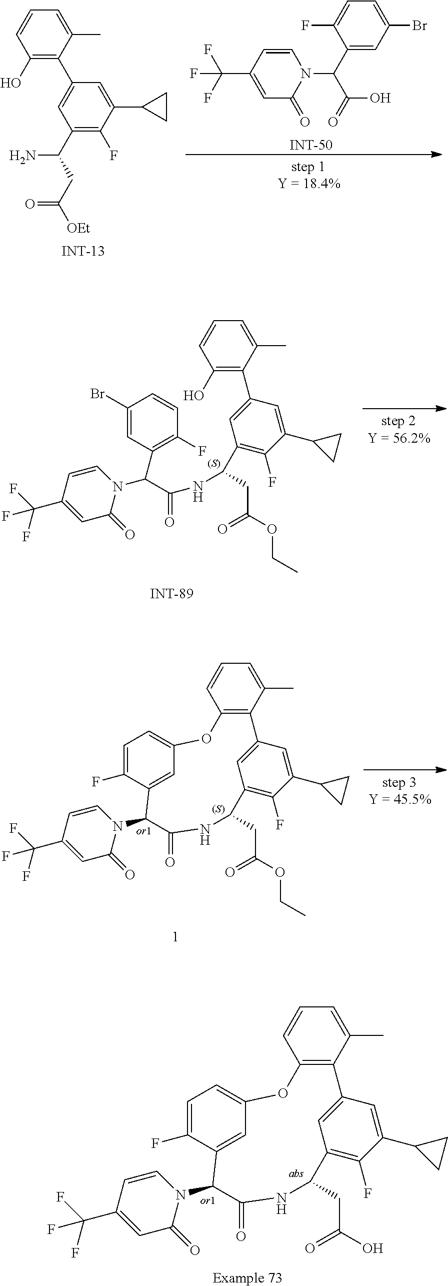

### Step 1: Preparation of ethyl (3S)-3-[2-(5-bromo-2-fluorophenyl)-2-[2-oxo-4-(trifluoromethyl) pyridin-1-yl]acetamido]-3-{5-cyclopropyl-4-fluoro-2′-hydroxy-6′-methyl-[1,1′-biphenyl]-3-yl}propanoate

To a stirred solution of ethyl (3S)-3-amino-3-{5-cyclopropyl-4-fluoro-2′-hydroxy-6′-methyl-[1,1′-biphenyl]-3-yl} propanoate (390 mg, 1.09 mmol, 1.00 equiv.) and (5-bromo-2-fluorophenyl) [2-oxo-4-(trifluoromethyl) pyridin-1-yl]acetic acid (430 mg, 1.09 mmol, 1.00 equiv.) in MeCN (5 mL) were added Et₃N (552 mg, 5.45 mmol, 5.00 equiv.), EDCI (627 mg, 3.27 mmol, 3.00 equiv.) and HOBT (294 mg, 2.18 mmol, 2.00 equiv.) at room temperature under nitrogen atmosphere. The reaction mixture was stirred for 16 h at 50° C. and then cooled to room temperature and concentrated. The residue was purified by reversed-phase flash chromatography (C18 gel; mobile phase, MeCN in water (0.1% NH₃·H₂O), 70% oto 90% gradient in 10 min; detector, UV 254 nm) to provide ethyl (3S)-3-[2-(5-bromo-2-fluorophenyl)-2-[2-oxo-4-(trifluoromethyl) pyridin-1-yl]acetamido]-3-{5-cyclopropyl-4-fluoro-2′-hydroxy-6′-methyl-[1,1′-biphenyl]-3-yl} propanoate (145 mg, 18.4%, mixture) as a white solid. LC-MS: (ES+H, m/z) [M+H]⁺=733.00.

### Step 2: Preparation of ethyl 2-[(14S,17S)-20-cyclopropyl-12,19-difluoro-3-methyl-15-oxo-14-[2-oxo-4-(trifluoromethyl) pyridin-1-yl]-8-oxa-16-azatetracyclo [16.3.1.1\hat{ }{9,13}.0\hat{ }{2,7}] tricosa-1(22),2(7),3,5,9,11,13(23),18,20-nonaen-17-yl]acetate

To a stirred solution of ethyl (3S)-3-[2-(5-bromo-2-fluorophenyl)-2-[2-oxo-4-(trifluoromethyl) pyridin-1-yl] acetamido]-3-{5-cyclopropyl-4-fluoro-2′-hydroxy-6′-methyl-[1,1′-biphenyl]-3-yl} propanoate (70 mg, 0.1 mmol, 1.00 equiv.) and CuI (9 mg, 0.05 mmol, 0.50 equiv.) in 1,4-dioxane (3 mL) were added K₂CO₃ (26 mg, 0.19 mmol, 2.00 equiv.) and (1R,2R)—N1,N2-dimethylcyclohexane-1,2-diamine (10 mg, 0.1 mmol, 1.00 equiv.) in portions at room temperature under nitrogen atmosphere. The resulting mixture was stirred for 2 h at 100° C. and then cooled to room temperature and concentrated. The residue was purified by reversed-phase flash chromatography (C18 gel; mobile phase, MeCN in water (0.1% HCOOH), 40% to 70% gradient in 20 min; detector, UV 220 nm) to provide ethyl 2-[(14S,17S)-20-cyclopropyl-12,19-difluoro-3-methyl-15-oxo-14-[2-oxo-4-(trifluoromethyl) pyridin-1-yl]-8-oxa-16-azatetracyclo [16.3.1.1\hat{ }{9,13}.0\hat{ }{2,7}]tricosa-1(22),2(7),3,5,9,11,13(23),18,20-nonaen-17-yl]acetate (35 mg, 56.2%) as a white solid. LC-MS: (ES+H, m/z) [M+H]⁺=653.15.

### Step 3: Preparation of [(14S,17S)-20-cyclopropyl-12,19-difluoro-3-methyl-15-oxo-14-[2-oxo-4-(trifluoromethyl) pyridin-1-yl]-8-oxa-16-azatetracyclo [16.3.1.1\hat{ }{9,13}.0\hat{ }{2,7}] tricosa-1(22),2(7),3,5,9,11,13(23),18,20-nonaen-17-yl]acetic acid

To a stirred solution of ethyl 2-[(14S,17S)-20-cyclopropyl-12,19-difluoro-3-methyl-15-oxo-14-[2-oxo-4-(trifluoromethyl) pyridin-1-yl]-8-oxa-16-azatetracyclo [16.3.1.1\hat{ }{9,13}.0\hat{ }{2,7}] tricosa-1(22),2(7),3,5,9,11,13(23),18,20-nonaen-17-yl]acetate (30 mg, 0.05 mmol, 1.00 equiv.) and LiOH·H₂O (7 mg, 0.19 mmol, 4.00 equiv.) in THF (5 mL) was added H₂O (1 mL) at room temperature. The resulting mixture was stirred for 2 h at room temperature and then was acidified to pH 5 with HCl (aq. 1 M) and concentrated. The residue was purified by Prep-HPLC (Column: Xbridge Phenyl OBD Column, 19*150 mm, 5 μm; Mobile Phase A: water (10 mmol/L NH₄HCO₃+0.05% NH₃H₂O, Mobile Phase B: MeCN; Flow rate: 60 mL/min; Gradient: 30% B to 45% B in 10 min; Wavelength: 254 nm/220 nm; RT1 (min) 10.02) to afford [(14S,17S)-20-cyclopropyl-12,19-difluoro-3-methyl-15-oxo-14-[2-oxo-4-(trifluoromethyl)pyridin-1-yl]-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid (13.3 mg, 45.5%) as a white solid. LC-MS: (ES+H, m/z) [M+H]⁺=625.20. ¹H NMR (400 MHz, DMSO-d₆) δ 9.40 (s, 1H), 7.61-7.53 (m, 2H), 7.40-7.30 (m, 2H), 7.25-7.14 (m, 2H), 6.81 (d, J=33.2 Hz, 2H), 6.60 (dd, J=6.9, 2.1 Hz, 1H), 6.35 (m, 2H), 5.97 (dd, J=6.2, 2.9 Hz, 1H), 5.47 (d, J=7.2 Hz, 1H), 2.46 (d, J=6.1 Hz, 2H), 2.15-2.03 (m, 1H), 1.91 (s, 3H), 1.00 (dd, J=8.4, 3.2 Hz, 2H), 0.79 (m, J=3.5 Hz, 1H), 0.73-0.66 (m, 1H). ¹⁹F NMR (377 MHz, DMSO-d₆) δ −65.44, −121.79, −129.51.

### Example 74

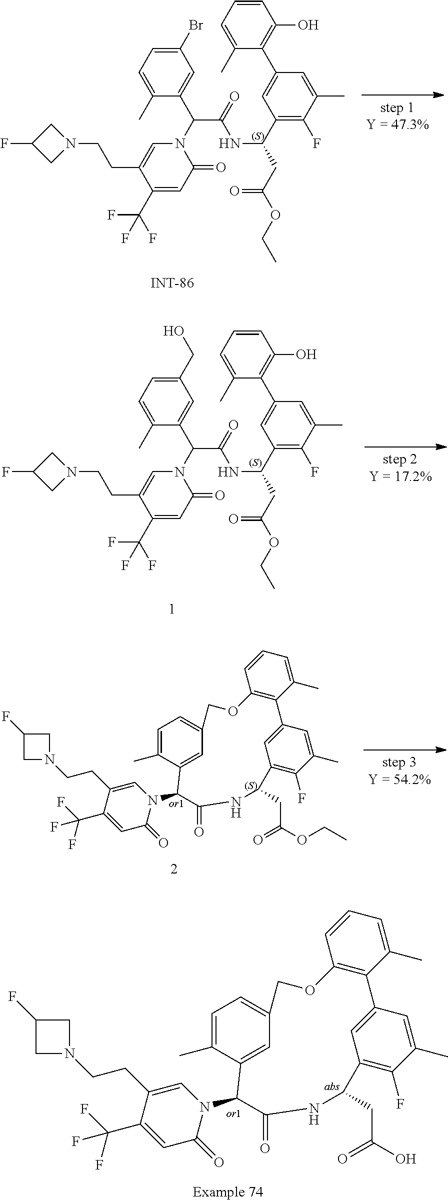

### Step 1: Preparation of ethyl (3S)-3-(4-fluoro-2′-hydroxy-5,6′-dimethyl-[1,1′-biphenyl]-3-yl)-3-(2-(5-(2-(3-fluoroazetidin-1-yl)ethyl)-2-oxo-4-(trifluoromethyl)pyridin-1(2H)-yl)-2-(5-(hydroxymethyl)-2-methylphenyl)acetamido)propanoate

A mixture of ethyl (3S)-3-[2-(5-bromo-2-methylphenyl)-2-{5-[2-(3-fluoroazetidin-1-yl)ethyl]-2-oxo-4-(trifluoromethyl)pyridin-1-yl}acetamido]-3-{4-fluoro-2′-hydroxy-5,6′-dimethyl-[1,1′-biphenyl]-3-yl}propanoate (360 mg, 0.44 mmol, 1.00 equiv.), (tributylstannyl)methanol (287 mg, 0.89 mmol, 2.00 equiv.) and 2nd generation XPhos Precatalyst (35 mg, 0.04 mmol, 0.10 equiv.) in 1,4-dioxane (3 mL) was stirred overnight at 90° C. under nitrogen atmosphere. The mixture was then cooled to room temperature and concentrated. The residue was purified by reversed-phase flash chromatography (C18 gel; mobile phase, MeCN in water (0.1% NH₃·H₂O), 30% to 60% gradient in 10 min; detector, UV 254 nm) to provide ethyl (3S)-3-{4-fluoro-2′-hydroxy-5,6′-dimethyl-[1,1′-biphenyl]-3-yl}-3-(2-{5-[2-(3-fluoroazetidin-1-yl)ethyl]-2-oxo-4-(trifluoromethyl)pyridin-1-yl}-2-[5-(hydroxymethyl)-2-methylphenyl]acetamido)propanoate (160 mg, 47.3%, mixture) as an off-white solid. LC-MS: (ES+H, m/z) [M+H]⁺=756.3.

### Step 2: Preparation of ethyl 2-[(15S,18S)-20-fluoro-15-{5-[2-(3-fluoroazetidin-1-yl)ethyl]-2-oxo-4-(trifluoromethyl)pyridin-1-yl}-3,13,21-trimethyl-16-oxo-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetate

A mixture of ethyl (3S)-3-{4-fluoro-2′-hydroxy-5,6′-dimethyl-[1,1′-biphenyl]-3-yl}-3-(2-{5-[2-(3-fluoroazetidin-1-yl)ethyl]-2-oxo-4-(trifluoromethyl)pyridin-1-yl}-2-[5-(hydroxymethyl)-2-methylphenyl]acetamido)propanoate (160 mg, 0.21 mmol, 1.00 equiv.) and 2-(tributyl-1\hat{ }[5]-phosphanylidene)acetonitrile (255 mg, 1.06 mmol, 5.00 equiv.) in toluene (2 mL) was stirred for 2 h at 80° C. under nitrogen atmosphere. The mixture was then to cool down to room temperature and concentrated. The residue was purified by reversed-phase flash chromatography (C18 gel; mobile phase, MeCN in water (0.1% NH₃·H₂O), 40% to 70% gradient in 10 min; detector, UV 254 nm) to provide ethyl 2-[(15S,18S)-20-fluoro-15-{5-[2-(3-fluoroazetidin-1-yl)ethyl]-2-oxo-4-(trifluoromethyl)pyridin-1-yl}-3,13,21-trimethyl-16-oxo-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetate (27 mg, 17.2%) as a white solid. LC-MS: (ES+H, m/z) [M+H]⁺=738.3.

### Step 3: Preparation of [(15S,18S)-20-fluoro-15-{5-[2-(3-fluoroazetidin-1-yl)ethyl]-2-oxo-4-(trifluoromethyl)pyridin-1-yl}-3,13,21-trimethyl-16-oxo-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetic acid

A mixture of ethyl 2-[(15S,18S)-20-fluoro-15-{5-[2-(3-fluoroazetidin-1-yl)ethyl]-2-oxo-4-(trifluoromethyl)pyridin-1-yl}-3,13,21-trimethyl-16-oxo-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetate (27 mg, 0.037 mmol, 1.00 equiv.) and LiOH·H₂O (4 mg, 0.11 mmol, 3.00 equiv.) in THF/H₂O=5/1 (0.6 mL) was stirred for 1 h at room temperature under nitrogen atmosphere. The resulting mixture was concentrated under reduced pressure and the crude product was purified by Prep-HPLC (Column: XSelect CSH Fluoro Phenyl, 30*150 mm, 5 μm; Mobile Phase A: water (0.1% HCOOH), Mobile Phase B: MeCN; Flow rate: 60 mL/min; Gradient: 15% B to 35% B in 10 min; Wavelength: 254 nm/220 nm; RT1 (min) 9.8) to afford [(15S,18S)-20-fluoro-15-{5-[2-(3-fluoroazetidin-1-yl)ethyl]-2-oxo-4-(trifluoromethyl)pyridin-1-yl}-3,13,21-trimethyl-16-oxo-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetic acid (10 mg, 54.2%) as a white solid. LC-MS: (ES+H, m/z) [M+H]⁺=710.25. ¹H NMR (300 MHz, CD₃OD) δ 7.34 (d, J=8.0 Hz, 1H), 7.27 (s, 1H), 7.24-7.12 (m, 3H), 7.08 (s, 1H), 6.95 (d, J=13.3 Hz, 2H), 6.87 (s, 1H), 6.84-6.76 (m, 1H), 6.62 (d, J=6.2 Hz, 1H), 5.75 (dd, J=8.8, 5.2 Hz, 1H), 5.29 (d, J=13.1 Hz, 1H), 5.10-4.93 (m, 2H), 3.53 (dd, J=15.8, 8.1 Hz, 2H), 3.27-3.10 (m, 2H), 2.79-2.54 (m, 4H), 2.49-2.38 (m, 2H), 2.31 (d, J=2.0 Hz, 3H), 2.08 (s, 3H), 1.96 (s, 3H). ¹⁹F NMR (282 MHz, CD₃OD) δ −64.91, −128.62, −180.40.

### Example 75

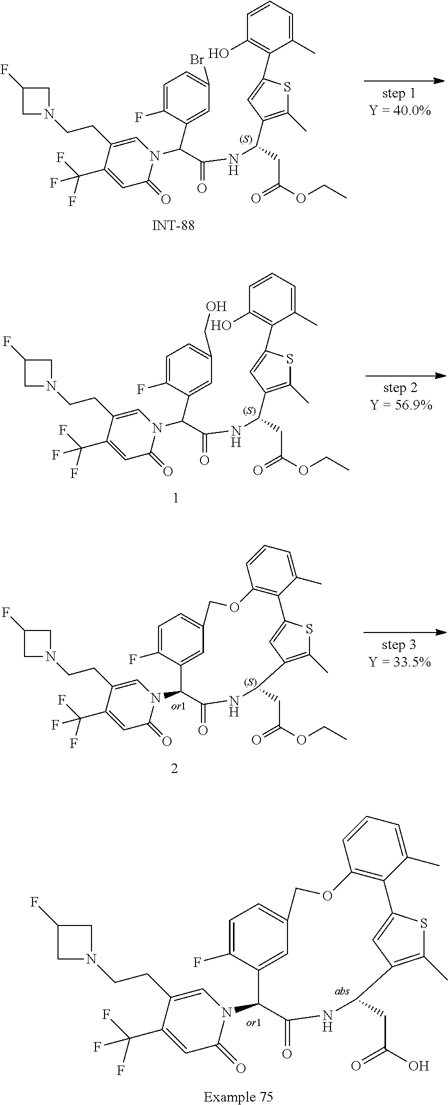

### Step 1: Preparation of ethyl (3S)-3-{2-[2-fluoro-5-(hydroxymethyl)phenyl]-2-{5-[2-(3-fluoroazetidin-1-yl)ethyl]-2-oxo-4-(trifluoromethyl)pyridin-1-yl}acetamido}-3-[5-(2-hydroxy-6-methylphenyl)-2-methylthiophen-3-yl]propanoate

A mixture of ethyl (3S)-3-[2-(5-bromo-2-fluorophenyl)-2-{5-[2-(3-fluoroazetidin-1-yl)ethyl]-2-oxo-4-(trifluoromethyl)pyridin-1-yl}acetamido]-3-[5-(2-hydroxy-6-methylphenyl)-2-methylthiophen-3-yl]propanoate (240 mg, 0.30 mmol, 1.00 equiv.), 2nd generation XPhos Precatalyst (24 mg, 0.03 mmol, 0.10 equiv.) and (tributylstannyl)methanol (290 mg, 0.90 mmol, 3.00 equiv.) in dioxane (5 mL) was stirred for 1 day at 80° C. under nitrogen atmosphere and then cooled to room temperature. The resulting mixture was diluted with water (15 mL). The resulting mixture was extracted with EtOAc (3×20 mL). The combined organic layers were washed with brine (3×20 mL), dried over anhydrous Na₂SO₄, filtered, and concentrated. The residue was purified by reversed-phase flash chromatography (C18 gel; mobile phase, MeCN in water (0.1% NH₃·H₂O), 40% to 80% gradient in 20 min; detector, UV 220 nm) to provide ethyl (3S)-3-{2-[2-fluoro-5-(hydroxymethyl)phenyl]-2-{5-[2-(3-fluoroazetidin-1-yl)ethyl]-2-oxo-4-(trifluoromethyl)pyridin-1-yl}acetamido}-3-[5-(2-hydroxy-6-methylphenyl)-2-methylthiophen-3-yl]propanoate (90 mg, 40.0%, mixture) as off-white solid. LC-MS: (ES+H, m/z) [M+H]⁺=748.20.

### Step 2: Preparation of ethyl 2-[(14S,17S)-19-fluoro-17-{5-[2-(3-fluoroazetidin-1-yl)ethyl]-2-oxo-4-(trifluoromethyl)pyridin-1-yl}-8,12-dimethyl-16-oxo-3-oxa-11-thia-15-azatetracyclo[16.3.1.1\hat{ }{10,13}.0\hat{ }{4,9}]tricosa-1(21),4(9),5,7,10(23),12,18(22),19-octaen-14-yl]acetate

A solution of ethyl (3S)-3-{2-[2-fluoro-5-(hydroxymethyl)phenyl]-2-{5-[2-(3-fluoroazetidin-1-yl)ethyl]-2-oxo-4-(trifluoromethyl)pyridin-1-yl}acetamido}-3-[5-(2-hydroxy-6-methylphenyl)-2-methylthiophen-3-yl]propanoate (90 mg, 0.12 mmol, 1.00 equiv.) and CMBP (145 mg, 0.60 mmol, 5.00 equiv.) in toluene (5 mL) was stirred overnight at 100° C. under nitrogen atmosphere. The resulting mixture was concentrated under reduced pressure. The residue was purified by reversed-phase flash chromatography (C18 gel; mobile phase, MeCN in water (0.1% NH₃·H₂O), 40% to 80% gradient in 20 min; detector, UV 220 nm) to provide ethyl 2-[(14S,17S)-19-fluoro-17-{5-[2-(3-fluoroazetidin-1-yl)ethyl]-2-oxo-4-(trifluoromethyl)pyridin-1-yl}-8,12-dimethyl-16-oxo-3-oxa-11-thia-15-azatetracyclo[16.3.1.1\hat{ }{10,13}.0\hat{ }{4,9}]tricosa-1(21),4(9),5,7,10(23),12,18(22),19-octaen-14-yl]acetate (50 mg, 56.9%) as light yellow solid. LC-MS: (ES+H, m/z) [M+H]⁺=730.30. ¹H NMR (400 MHz, CD₃OD) δ 7.49-7.40 (m, 2H), 7.27-7.14 (m, 3H), 7.01 (d, J=7.3 Hz, 2H), 6.92 (dd, J=7.6, 3.9 Hz, 1H), 6.88 (s, 1H), 6.83 (s, 1H), 5.30 (dd, J=8.6, 7.2 Hz, 1H), 5.18-5.04 (m, 2H), 4.98 (t, J=4.8 Hz, 1H), 4.18-4.03 (m, 2H), 3.50-3.36 (m, 2H), 3.18-2.99 (m, 2H), 2.94-2.74 (m, 2H), 2.61-2.40 (m, 7H), 2.36 (s, 3H), 1.18 (t, J=7.1 Hz, 3H). ¹⁹F NMR (377 MHz, CD₃OD) δ −64.90, −118.85, −180.46.

### Step 3: Preparation of [(14S,17S)-19-fluoro-17-{5-[2-(3-fluoroazetidin-1-yl)ethyl]-2-oxo-4-(trifluoromethyl)pyridin-1-yl}-8,12-dimethyl-16-oxo-3-oxa-11-thia-15-azatetracyclo[16.3.1.1\hat{ }{10,13}.0\hat{ }{4,9}]tricosa-1(21),4(9),5,7,10(23),12,18(22),19-octaen-14-yl]acetic acid

A mixture of ethyl 2-[(14S,17S)-19-fluoro-17-{5-[2-(3-fluoroazetidin-1-yl)ethyl]-2-oxo-4-(trifluoromethyl)pyridin-1-yl}-8,12-dimethyl-16-oxo-3-oxa-11-thia-15-azatetracyclo[16.3.1.1\hat{ }{10,13}.0\hat{ }{4,9}]tricosa-1(21),4(9),5,7,10(23),12,18(22),19-octaen-14-yl]acetate (45 mg, 0.06 mmol, 1.00 equiv.) and LiOH·H₂O (10 mg, 0.25 mmol, 4.00 equiv.) in THF (4 mL) and H₂O (1 mL) was stirred overnight at room temperature under nitrogen atmosphere. The mixture was acidified to pH 5 with HCl (aq. 1 M). The resulting mixture was concentrated under reduced pressure. The crude product (40 mg) was purified by Prep-HPLC (Column: XSelect CSH Prep C18 OBD Column, 30*150 mm, 5 μm; Mobile Phase A: water (0.1% HCOOH), Mobile Phase B: MeCN; Flow rate: 60 mL/min; Gradient: 17% B to 37% B in 10 min; Wavelength: 254 nm/220 nm; RT1 (min) 9.99) to afford [(14S,17S)-19-fluoro-17-{5-[2-(3-fluoroazetidin-1-yl)ethyl]-2-oxo-4-(trifluoromethyl)pyridin-1-yl}-8,12-dimethyl-16-oxo-3-oxa-11-thia-15-azatetracyclo[16.3.1.1\hat{ }{10,13}.0\hat{ }{4,9}]tricosa-1(21),4(9),5,7,10(23),12,18(22),19-octaen-14-yl]acetic acid (15.2 mg, 33.5%) as a white solid. LC-MS: (ES+H, m/z) [M+H]⁺=702.10. ¹H NMR (300 MHz, DMSO-d₆) δ 9.34 (d, J=5.9 Hz, 1H), 7.52-7.43 (m, 1H), 7.33-7.21 (m, 3H), 7.14-7.01 (m, 2H), 6.96-6.86 (m, 2H), 6.82 (s, 1H), 6.76 (s, 1H), 5.23 (d, J=13.1 Hz, 1H), 5.14-5.01 (m, 2H), 4.96-4.73 (m, 1H), 3.21-3.11 (m, 2H), 2.87-2.57 (m, 4H), 2.47 (s, 3H), 2.39 (t, J=5.8 Hz, 1H), 2.32 (d, J=3.3 Hz, 6H). ¹⁹F NMR (282 MHz, DMSO-d₆) δ −62.49, −117.79, −176.88.

### Example 76

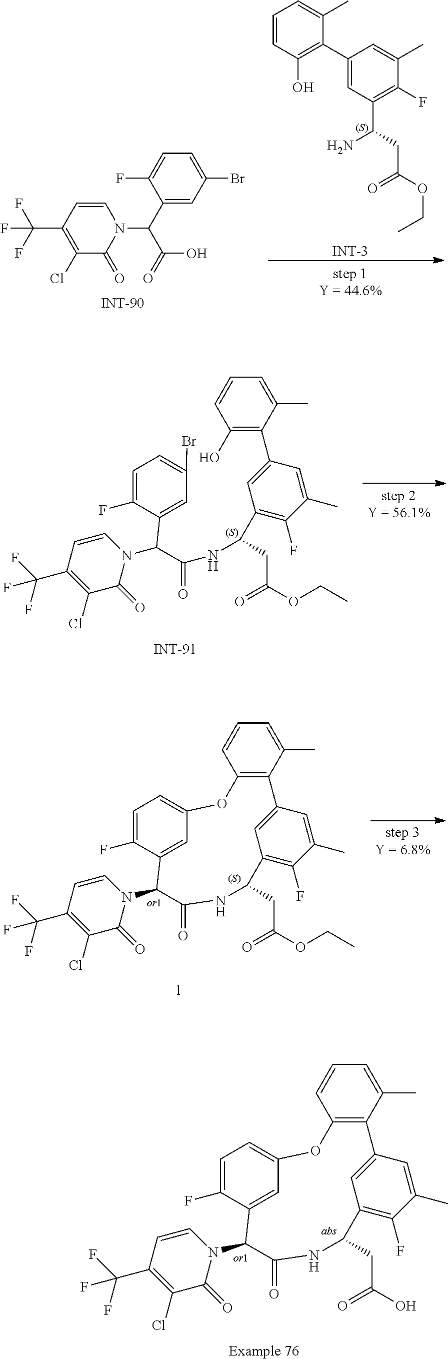

### Step 1: Preparation of ethyl (3S)-3-(2-(5-bromo-2-fluorophenyl)-2-(3-chloro-2-oxo-4-(trifluoromethyl)pyridin-1(2H)-yl)acetamido)-3-(4-fluoro-2′-hydroxy-5,6′-dimethyl-[1,1′-biphenyl]-3-yl)propanoate

A mixture of ethyl (3S)-3-amino-3-{4-fluoro-2′-hydroxy-5,6′-dimethyl-[1,1′-biphenyl]-3-yl}propanoate hydrochloride (489 mg, 1.33 mmol, 1.00 equiv.) and NEt₃ (404 mg, 3.99 mmol, 3.00 equiv.) in MeCN (5 mL) was stirred for 30 min at room temperature under nitrogen atmosphere. The resulting mixture was concentrated under reduced pressure. This resulted in ethyl (3S)-3-amino-3-{4-fluoro-2′-hydroxy-5,6′-dimethyl-[1,1′-biphenyl]-3-yl}propanoate. A mixture of (5-bromo-2-fluorophenyl)[3-chloro-2-oxo-4-(trifluoromethyl)pyridin-1-yl]acetic acid; trifluoroacetaldehyde (700 mg, 1.33 mmol, 1.00 equiv.), EDCI (382 mg, 1.99 mmol, 1.50 equiv.), HOBT (234 mg, 1.73 mmol, 1.30 equiv.) and DIEA (859 mg, 6.65 mmol, 5.00 equiv.) in MeCN (5 mL) was stirred for 40 min at 40° C. under nitrogen atmosphere. To the above mixture was added ethyl (3S)-3-amino-3-{4-fluoro-2′-hydroxy-5,6′-dimethyl-[1,1′-biphenyl]-3-yl}propanoate in MeCN (3 mL) at room temperature. The resulting mixture was stirred overnight at 40° C. The resulting mixture was concentrated under reduced pressure. The residue was purified by reversed-phase flash chromatography (C18 gel; mobile phase, MeCN in water (0.1% NH₃·H₂O), 10% to 50% gradient in 10 min; detector, UV 254 nm) to provide ethyl (3S)-3-[2-(5-bromo-2-fluorophenyl)-2-[3-chloro-2-oxo-4-(trifluoromethyl)pyridin-1-yl]acetamido]-3-{4-fluoro-2′-hydroxy-5,6′-dimethyl-[1,1′-biphenyl]-3-yl}propanoate (440 mg, 44.6%, mixture) as a white solid. LC-MS: (ES−H, m/z) [M−H]⁻=739.05.

### Step 2: Preparation of ethyl 2-((5S,8S)-5-(3-chloro-2-oxo-4-(trifluoromethyl)pyridin-1(2H)-yl)-14,44-difluoro-15,26-dimethyl-6-oxo-3-oxa-7-aza-1,4(1,3),2(1,2)-tribenzenacyclooctaphane-8-yl)acetate

A mixture of ethyl (3S)-3-[2-(5-bromo-2-fluorophenyl)-2-[3-chloro-2-oxo-4-(trifluoromethyl)pyridin-1-yl]acetamido]-3-{4-fluoro-2′-hydroxy-5,6′-dimethyl-[1,1′-biphenyl]-3-yl}propanoate (100 mg, 0.13 mmol, 1.00 equiv.), (1R,2R)—N1,N2-dimethylcyclohexane-1,2-diamine (19 mg, 0.13 mmol, 1.00 equiv.), CuI (12 mg, 0.06 mmol, 0.50 equiv.) and K₂CO₃ (37 mg, 0.27 mmol, 2.00 equiv.) in 1,4-dioxane (3 mL) was stirred for 2 h at 100° C. under nitrogen atmosphere. The resulting mixture was concentrated under reduced pressure. The residue was purified by reversed-phase flash chromatography (C18 silica gel; mobile phase, MeCN in water (0.11% NH₃·H₂O), 50% to 80% gradient in 10 min; detector, UV 254 nm) to provide ethyl 2-[(14S,17S)-14-[3-chloro-2-oxo-4-(trifluoromethyl)pyridin-1-yl]-12,19-difluoro-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (50 mg, 56.1%) as a white solid. LC-MS: (ES+H, m/z) [M+H]⁺=661.2. 1H NMR (300 MHz, MeOD) δ 7.55 (ddd, J=9.1, 4.2, 3.0 Hz, 1H), 7.47-7.40 (m, 1H), 7.34 (t, J=7.8 Hz, 1H), 7.30-7.25 (m, 1H), 7.22-7.11 (m, 2H), 7.01-6.94 (m, 1H), 6.78 (s, 1H), 6.51 (d, J=7.4 Hz, 1H), 6.43 (dd, J=6.3, 2.2 Hz, 1H), 6.05 (dd, J=6.1, 2.9 Hz, 1H), 5.69 (t, J=7.9 Hz, 1H), 4.11 (qd, J=7.1, 2.1 Hz, 2H), 2.86 (dd, J=15.4, 8.3 Hz, 1H), 2.73 (dd, J=15.5, 7.5 Hz, 1H), 2.34 (d, J=2.1 Hz, 3H), 1.99 (s, 3H), 1.19 (t, J=7.1 Hz, 3H). ¹⁹F NMR (282 MHz, MeOD) δ −66.06, −123.33, −126.75.

### Step 3: Preparation of 2-((5S,8S)-5-(3-chloro-2-oxo-4-(trifluoromethyl)pyridin-1(2H)-yl)-14,44-difluoro-15,26-dimethyl-6-oxo-3-oxa-7-aza-1,4(1,3),2(1,2)-tribenzenacyclooctaphane-8-yl)acetic acid

A mixture of ethyl 2-[(14S,17S)-14-[3-chloro-2-oxo-4-(trifluoromethyl)pyridin-1-yl]-12,19-difluoro-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (50 mg, 0.07 mmol, 1.00 equiv.) and LiOH·H₂O (9 mg, 0.22 mmol, 3.00 equiv.) in THF/H₂O=5/1 (1.2 mL) was stirred for 2 h at room temperature under nitrogen atmosphere. The resulting mixture was concentrated under reduced pressure. The crude product (53 mg) was purified by Prep-HPLC (Column: XBridge Prep OBD C18 Column, 30*150 mm, 5 μm; Mobile Phase A: water (10 mmol/L NH₄HCO₃, Mobile Phase B: MeCN;
Flow rate: 60 mL/min; Gradient: 27% B to 42% B in 10 min; Wavelength: 254 nm/220 nm; RT1 (min) 10.58) to afford 2-((5S,8S)-5-(3-chloro-2-oxo-4-(trifluoromethyl)pyridin-1(2H)-yl)-14,44-difluoro-15,26-dimethyl-6-oxo-3-oxa-7-aza-1,4(1,3),2(1,2)-tribenzenacyclooctaphane-8-yl)acetic acid (3.4 mg, 6.8%) as a white solid. LC-MS: (ES+H, m/z) [M+H]⁺=633.10. ¹H NMR (300 MHz, CD₃OD) δ 7.54 (dt, J=9.1, 3.6 Hz, 1H), 7.42 (d, J=7.8 Hz, 1H), 7.33 (d, J=7.7 Hz, 1H), 7.28 (d, J=7.9 Hz, 1H), 7.20-7.11 (m, 2H), 6.93 (d, J=6.4 Hz, 1H), 6.82 (s, 1H), 6.53-6.46 (m, 2H), 6.08 (dd, J=6.2, 3.0 Hz, 1H), 5.70 (t, J=6.9 Hz, 1H), 2.67-2.56 (m, 2H), 2.33 (d, J=2.0 Hz, 3H), 1.99 (s, 3H). ¹⁹F NMR (282 MHz, MeOD) δ −65.73, −123.31, −126.74.

### Example 77

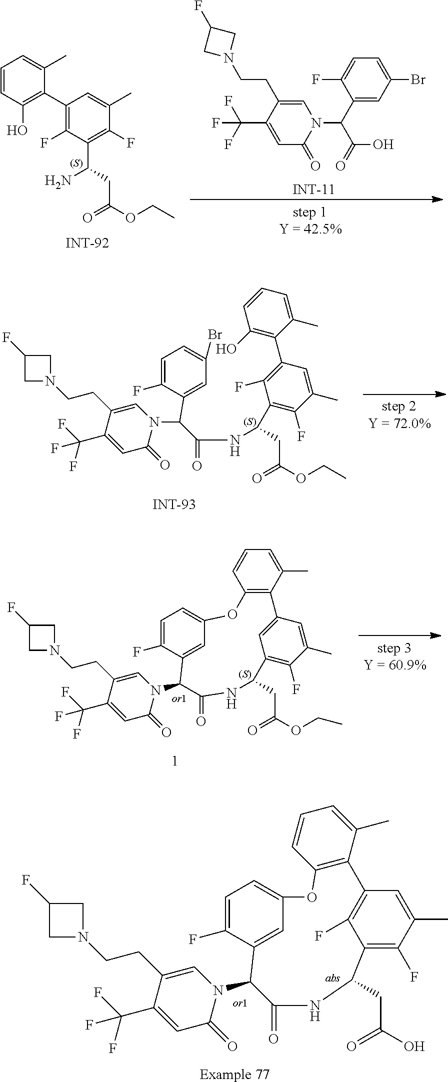

### Step 1: Preparation of ethyl (3S)-3-[2-(5-bromo-2-fluorophenyl)-2-{5-[2-(3-fluoroazetidin-1-yl)ethyl]-2-oxo-4-(trifluoromethyl)pyridin-1-yl}acetamido]-3-{2,4-difluoro-2′-hydroxy-5,6′-dimethyl-[1,1′-biphenyl]-3-yl}propanoate

To a stirred mixture of ethyl (3S)-3-amino-3-{2,4-difluoro-2′-hydroxy-5,6′-dimethyl-[1,1′-biphenyl]-3-yl}propanoate (750 mg, 2.14 mmol, 1.00 equiv.) and (5-bromo-2-fluorophenyl)({5-[2-(3-fluoroazetidin-1-yl)ethyl]-2-oxo-4-(trifluoromethyl)pyridin-1-yl})acetic acid (1.06 g, 2.14 mmol, 1.00 equiv.) in DMF (10 mL) were added HATU (1.63 g, 4.29 mmol, 2.00 equiv.) and DIEA (1.11 g, 8.58 mmol, 4.00 equiv.) at room temperature under nitrogen atmosphere. The resulting mixture was stirred for 4 h at room temperature under nitrogen atmosphere. The resulting mixture was diluted with water (50 mL). The resulting mixture was extracted with EtOAc (3×80 mL). The combined organic layers were washed with water (2×100 mL), dried over anhydrous Na₂SO₄, filtered, and concentrated. The residue was purified by reversed-phase flash chromatography (C18 gel; mobile phase, MeCN in water (10 mmol/L NH₄HCO₃), 30% to 70% gradient in 25 min; detector, UV 220 nm) to afford ethyl (3S)-3-[2-(5-bromo-2-fluorophenyl)-2-{5-[2-(3-fluoroazetidin-1-yl)ethyl]-2-oxo-4-(trifluoromethyl)pyridin-1-yl}acetamido]-3-{2,4-difluoro-2′-hydroxy-5,6′-dimethyl-[1,1′-biphenyl]-3-yl}propanoate (755 mg, 42.5%, mixture) as a yellow solid. LC-MS: (ES+H, m/z) [M+H]⁺=826

### Step 2: Preparation of ethyl 2-[(14S,17S)-12,19,22-trifluoro-14-{5-[2-(3-fluoroazetidin-1-yl)ethyl]-2-oxo-4-(trifluoromethyl)pyridin-1-yl}-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate

To a stirred mixture of ethyl (3S)-3-[2-(5-bromo-2-fluorophenyl)-2-{5-[2-(3-fluoroazetidin-1-yl)ethyl]-2-oxo-4-(trifluoromethyl)pyridin-1-yl}acetamido]-3-{2,4-difluoro-2′-hydroxy-5,6′-dimethyl-[1,1′-biphenyl]-3-yl}propanoate (200 mg, 0.24 mmol, 1.00 equiv.) and (1R,2R)—N1,N2-dimethylcyclohexane-1,2-diamine (33.93 mg, 0.24 mmol, 1.00 equiv.) in 1,4-dioxane (5 mL) were added CuI (23 mg, 0.12 mmol, 0.50 equiv.) and K₂CO₃ (66 mg, 0.48 mmol, 2.00 equiv.) in portions at room temperature under nitrogen atmosphere. The resulting mixture was stirred overnight at 100° C. under nitrogen atmosphere and then cooled to room temperature. The resulting mixture was filtered, and the filter cake was washed with ethyl acetate. The filtrate was concentrated under reduced pressure. The residue was purified by reversed-phase flash chromatography (C18 gel; mobile phase, MeCN in water (10 mmol/L NH₄HCO₃), 30% to 70% gradient in 25 min; detector, UV 220 nm) to afford ethyl 2-[(14S,17S)-12,19,22-trifluoro-14-{5-[2-(3-fluoroazetidin-1-yl)ethyl]-2-oxo-4-(trifluoromethyl)pyridin-1-yl}-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (130 mg, 72.0%) as a yellow solid. LC-MS: (ES+H, m/z) [M+H]⁺=746.2.

### Step 3: Preparation of [(14S,17S)-12,19,22-trifluoro-14-{5-[2-(3-fluoroazetidin-1-yl)ethyl]-2-oxo-4-(trifluoromethyl)pyridin-1-yl}-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid

To a stirred solution of ethyl 2-[(14S,17S)-12,19,22-trifluoro-14-{5-[2-(3-fluoroazetidin-1-yl)ethyl]-2-oxo-4-(trifluoromethyl)pyridin-1-yl}-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (120 mg, 0.16 mmol, 1.00 equiv.) in THF (3 mL) was added LiOH·H₂O (13 mg, 0.32 mmol, 2.00 equiv.) in water (1 mL) dropwise at room temperature under nitrogen atmosphere. The resulting mixture was stirred for 2 h at room temperature under nitrogen atmosphere and then concentrated. The crude product was purified by Prep-HPLC (Column: XBridge Prep OBD C18 Column, 30*150 mm, 10 μm; Mobile Phase A: water (10 mmol/L NH₄HCO₃+0.05% NH₃H₂O), Mobile Phase B: MeOH; Flow rate: 60 mL/min; Gradient: 53% B to 69% B in 8 min; Wavelength: 254 nm/220 nm; RT1 (min) 9.13), to afford [(14S,17S)-12,19,22-trifluoro-14-{5-[2-(3-fluoroazetidin-1-yl)ethyl]-2-oxo-4-(trifluoromethyl)pyridin-1-yl}-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid (73.5 mg, 60.9%) as a white solid. LC-MS: (ES+H, m/z) [M+H]⁺=718.20. ¹H NMR (400 MHz, DMSO-d₆) δ 9.21 (d, J=6.9 Hz, 1H), 7.66-7.55 (m, 2H), 7.41 (t, J=7.9 Hz, 1H), 7.29-7.17 (m, 3H), 7.12 (t, J=8.1 Hz, 1H), 6.79 (s, 1H), 6.70 (s, 1H), 5.89 (dd, J=6.4, 2.9 Hz, 1H), 5.48 (d, J=7.5 Hz, 1H), 4.99-4.71 (m, 1H), 3.32-3.16 (m, 2H), 2.91-2.64 (m, 4H), 2.45-2.29 (m, 4H), 2.26 (s, 3H), 1.98 (s, 3H). ¹⁹F NMR (400 MHz, DMSO-d₆) δ −62.640, −116.300, −116.761, −121.054, −176.934.

### Example 78

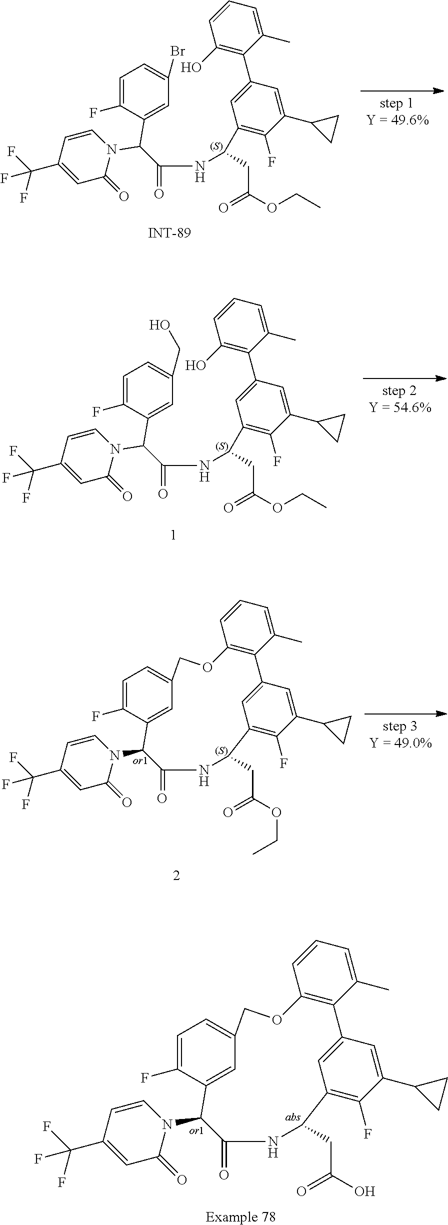

### Step 1: Preparation of ethyl (3S)-3-{5-cyclopropyl-4-fluoro-2′-hydroxy-6′-methyl-[1,1′-biphenyl]-3-yl}-3-{2-[2-fluoro-5-(hydroxymethyl)phenyl]-2-[2-oxo-4-(trifluoromethyl)pyridin-1-yl]acetamido}propanoate

A mixture of ethyl (3S)-3-[2-(5-bromo-2-fluorophenyl)-2-[2-oxo-4-(trifluoromethyl)pyridin-1-yl]acetamido]-3-{5-cyclopropyl-4-fluoro-2′-hydroxy-6′-methyl-[1,1′-biphenyl]-3-yl}propanoate (80 mg, 0.11 mmol, 1.00 equiv.), 3nd generation XPhos precatalyst (9 mg, 0.01 mmol, 0.10 equiv.) and (tributylstannyl)methanol (105 mg, 0.33 mmol, 3.00 equiv.) in 1,4-dioxane (4 mL) was stirred overnight at 80° C. under nitrogen atmosphere and then cooled to room temperature. The resulting mixture was diluted with water (3 mL). The resulting mixture was extracted with EtOAc (3×10 mL). The combined organic layers were washed with brine (2×5 mL), dried over anhydrous Na₂SO₄, filtered, and concentrated. The residue was purified by reversed-phase flash chromatography (C18 gel; mobile phase, MeCN in water (0.1% NH₃·H₂O), 40% to 70% gradient in 15 min; detector, UV 220 nm) to provide ethyl (3S)-3-{5-cyclopropyl-4-fluoro-2′-hydroxy-6′-methyl-[1,1′-biphenyl]-3-yl}-3-{2-[2-fluoro-5-(hydroxymethyl)phenyl]-2-[2-oxo-4-(trifluoromethyl)pyridin-1-yl]acetamido}propanoate (37 mg, 49.6%, mixture) as a white solid. LC-MS: (ES+H, m/z) [M+H]⁺=685.30. ¹H NMR (400 MHz, CD₃OD) δ 7.51 (d, J=7.2 Hz, 1H), 7.47-7.35 (m, 1H), 7.22-7.09 (m, 1H), 7.06-6.88 (m, 3H), 6.85-6.65 (m, 4H), 6.49-6.35 (m, 1H), 5.73 (q, J=7.7 Hz, 1H), 4.71-4.62 (m, 1H), 4.39 (s, 1H), 4.21-3.92 (m, 2H), 2.96-2.82 (m, 2H), 2.11 (ddd, J=14.0, 7.0, 4.2 Hz, 1H), 1.92 (d, J=6.3 Hz, 3H), 1.36-1.24 (m, 1H), 1.22-1.08 (m, 3H), 1.01-0.92 (m, 2H), 0.71 (d, J=7.1 Hz, 2H). ¹⁹F NMR (377 MHz, CD₃OD) δ −68.23, −119.66, −130.00.

### Step 2: Preparation of ethyl 2-[(15S,18S)-21-cyclopropyl-13,20-difluoro-3-methyl-16-oxo-15-[2-oxo-4-(trifluoromethyl)pyridin-1-yl]-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetate

A mixture of ethyl (3S)-3-{5-cyclopropyl-4-fluoro-2′-hydroxy-6′-methyl-[1,1′-biphenyl]-3-yl}-3-{2-[2-fluoro-5-(hydroxymethyl)phenyl]-2-[2-oxo-4-(trifluoromethyl)pyridin-1-yl]acetamido}propanoate (32 mg, 0.05 mmol, 1.00 equiv.), DBU (14 mg, 0.10 mmol, 2.00 equiv.) and 2-(tributyl-lambda5-phosphanylidene)acetonitrile (56 mg, 0.24 mmol, 5.00 equiv.) in toluene (3 mL) was stirred overnight at 80° C. under nitrogen atmosphere. The resulting mixture was diluted with water (3 mL). The resulting mixture was extracted with EtOAc (3×15 mL). The resulting mixture was concentrated under reduced pressure. The residue was purified by reversed-phase flash chromatography (C18 gel; mobile phase, MeCN in water (0.1% NH₃·H₂O), 40% to 70% gradient in 15 min; detector, UV 220 nm) to provide ethyl 2-[(15S,18S)-21-cyclopropyl-13,20-difluoro-3-methyl-16-oxo-15-[2-oxo-4-(trifluoromethyl)pyridin-1-yl]-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetate (17 mg, 54.6%) as off-white solid. LC-MS: (ES+H, m/z) [M+H]⁺=667.30.

### Step 3: Preparation of [(15S,18S)-21-cyclopropyl-13,20-difluoro-3-methyl-16-oxo-15-[2-oxo-4-(trifluoromethyl)pyridin-1-yl]-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetic acid

A mixture of ethyl 2-[(15S,18S)-21-cyclopropyl-13,20-difluoro-3-methyl-16-oxo-15-[2-oxo-4-(trifluoromethyl)pyridin-1-yl]-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetate (15 mg, 0.02 mmol, 1.00 equiv.) and LiOH·H₂O (4 mg, 0.09 mmol, 4.00 equiv.) in THF (2 mL) and H₂O (500 μL) was stirred overnight at room temperature under nitrogen atmosphere. The mixture was acidified to pH 5 with HCl (aq. 1 M). The resulting mixture was concentrated under reduced pressure. The crude product (15 mg) was purified by Prep-HPLC (Column: Xbridge Phenyl OBD Column, 19*150 mm, 5 μm; Mobile Phase A: water (10 mmol/L NH₄HCO₃+0.05% NH₃H₂O, Mobile Phase B: MeCN; Flow rate: 60 mL/min; Gradient: 30% B to 45% B in 10 min; Wavelength: 254 nm/220 nm; RT1 (min) 9.68) to afford [(15S,18S)-21-cyclopropyl-13,20-difluoro-3-methyl-16-oxo-15-[2-oxo-4-(trifluoromethyl)pyridin-1-yl]-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetic acid (7.3 mg, 49.0%) as a white solid. LC-MS: (ES+H, m/z) [M+H]⁺=639.10. ¹H NMR (400 MHz, CD₃OD) δ 7.46 (t, J=6.2 Hz, 1H), 7.30 (dd, J=16.0, 7.0 Hz, 2H), 7.21-7.02 (m, 4H), 6.87-6.77 (m, 2H), 6.62 (d, J=6.7 Hz, 2H), 6.38 (dd, J=7.4, 2.0 Hz, 1H), 5.76 (dd, J=8.8, 4.9 Hz, 1H), 5.29 (d, J=13.2 Hz, 1H), 4.97 (d, J=13.2 Hz, 1H), 2.77-2.58 (m, 2H), 2.18-2.10 (m, 1H), 1.93 (s, 3H), 1.29 (s, 1H), 0.99 (d, J=8.6 Hz, 2H), 0.77-0.66 (m, 2H). ¹⁹F NMR (377 MHz, CD₃OD) δ −88.24, −118.44, −131.39.

### Example 79

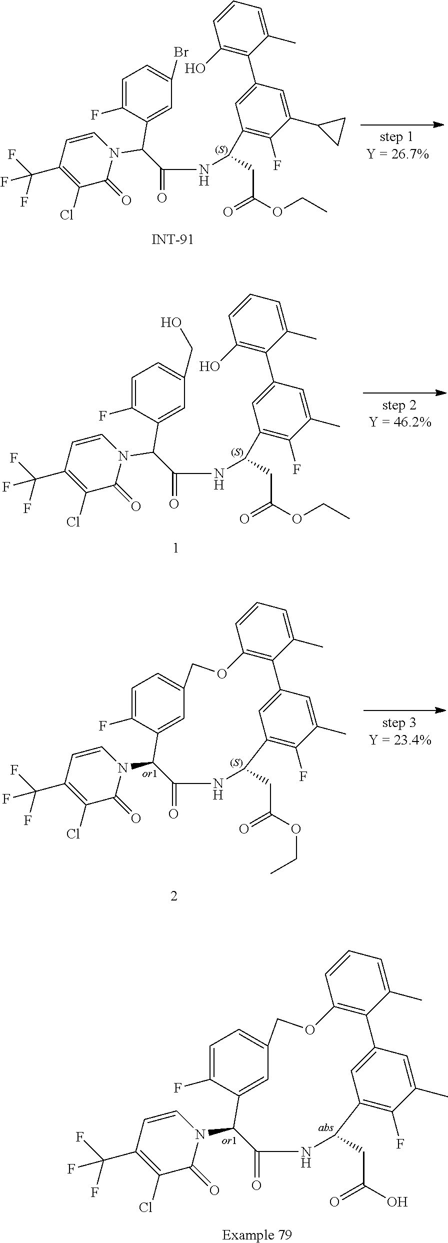

### Step 1: Preparation of ethyl (3S)-3-{2-[3-chloro-2-oxo-4-(trifluoromethyl)pyridin-1-yl]-2-[2-fluoro-5-(hydroxymethyl)phenyl]acetamido}-3-{4-fluoro-2′-hydroxy-5,6′-dimethyl-[1,1′-biphenyl]-3-yl}propanoate

A mixture of ethyl (3S)-3-[2-(5-bromo-2-fluorophenyl)-2-[3-chloro-2-oxo-4-(trifluoromethyl)pyridin-1-yl]acetamido]-3-{4-fluoro-2′-hydroxy-5,6′-dimethyl-[1,1′-biphenyl]-3-yl}propanoate (400 mg, 0.53 mmol, 1 equiv.), (tributylstannyl)methanol (346 mg, 1.07 mmol, 2.00 equiv.) and 2nd generation XPhos precatalyst (42 mg, 0.05 mmol, 0.10 equiv.) in 1,4-dioxane (4 mL) was stirred for 4 h at 90° C. under nitrogen atmosphere. The mixture was allowed to cool down to room temperature. The resulting mixture was concentrated under reduced pressure. The residue was purified by reversed-phase flash chromatography (C18 gel; mobile phase, MeCN in water (0.1% NH₃·H₂O), 40% to 60% gradient in 10 min; detector, UV 254 nm) to provide ethyl (3S)-3-{2-[3-chloro-2-oxo-4-(trifluoromethyl)pyridin-1-yl]-2-[2-fluoro-5-(hydroxymethyl)phenyl]acetamido}-3-{4-fluoro-2′-hydroxy-5,6′-dimethyl-[1,1′-biphenyl]-3-yl}propanoate (100 mg, 26.7%) as a white solid. LC-MS: (ES+H, m/z) [M+H]⁺=693.2.

### Step 2: Preparation of ethyl 2-[(15S,18S)-15-[3-chloro-2-oxo-4-(trifluoromethyl)pyridin-1-yl]-13,20-difluoro-3,21-dimethyl-16-oxo-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetate

A mixture of ethyl (3S)-3-{2-[3-chloro-2-oxo-4-(trifluoromethyl)pyridin-1-yl]-2-[2-fluoro-5-(hydroxymethyl)phenyl]acetamido}-3-{4-fluoro-2′-hydroxy-5,6′-dimethyl-[1,1′-biphenyl]-3-yl}propanoate (80 mg, 0.11 mmol, 1.00 equiv.) and CMBP (139 mg, 0.57 mmol, 5.00 equiv.) in toluene (3 mL) was stirred for 1 h at 100° C. under nitrogen atmosphere. The mixture was allowed to cool down to room temperature. The resulting mixture was concentrated under reduced pressure. The residue was purified by reversed-phase flash chromatography (C18 gel; mobile phase, MeCN in water (0.1% NH₃·H₂O), 50% to 70% gradient in 10 min; detector, UV 254 nm) to provide ethyl 2-[(15S,18S)-15-[3-chloro-2-oxo-4-(trifluoromethyl)pyridin-1-yl]-13,20-difluoro-3,21-dimethyl-16-oxo-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetate (36 mg, 46.2%) as an off-white solid. LC-MS: (ES+H, m/z) [M+H]⁺=675.2.

### Step 3: Preparation of [(15R*,18S)-15-[3-chloro-2-oxo-4-(trifluoromethyl)pyridin-1-yl]-13,20-difluoro-3,21-dimethyl-16-oxo-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetic acid

A mixture of ethyl 2-[(15S,18S)-15-[3-chloro-2-oxo-4-(trifluoromethyl)pyridin-1-yl]-13,20-difluoro-3,21-dimethyl-16-oxo-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetate (30 mg, 0.04 mmol, 1.00 equiv.) and LiOH·H₂O (5 mg, 0.13 mmol, 3.00 equiv.) in THF/H₂O=5/1 (0.6 mL) was stirred for 1 h at room temperature under nitrogen atmosphere. The resulting mixture was concentrated under reduced pressure. The crude product (35 mg) was purified by Prep-HPLC (Column: XBridge Shield RP18 OBD Column 30*150 mm, 5 μm; Mobile Phase A: water (0.1% FA), Mobile Phase B: MeCN; Flow rate: 60 mL/min; Gradient: 48% B to 68% B in 8 min; Wavelength: 254 nm/220 nm; RT1 (min) 9.08) to afford [(15R*,18S)-15-[3-chloro-2-oxo-4-(trifluoromethyl)pyridin-1-yl]-13,20-difluoro-3,21-dimethyl-16-oxo-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetic acid (6.8 mg, 23.4%) as a white solid. LC-MS: (ES−H, m/z) [M−H]⁻=645.05. ¹H NMR (300 MHz, CD₃OD) δ 7.48 (ddd, J=7.9, 5.0, 2.2 Hz, 1H), 7.29 (dd, J=7.2, 2.1 Hz, 1H), 7.23 (d, J=7.4 Hz, 1H), 7.20-7.10 (m, 3H), 7.08 (d, J=1.7 Hz, 1H), 6.99-6.91 (m, 1H), 6.86-6.77 (m, 1H), 6.60 (dd, J=6.7, 2.1 Hz, 1H), 6.47 (d, J=7.5 Hz, 1H), 5.77 (dd, J=9.0, 5.4 Hz, 1H), 5.30 (d, J=13.2 Hz, 1H), 4.96 (d, J=13.2 Hz, 1H), 2.83-2.64 (m, 2H), 2.31 (d, J=2.1 Hz, 3H), 1.96 (s, 3H). ¹⁹F NMR (282 MHz, CD₃OD) δ −65.73, −118.75, −128.37.

### Example 80

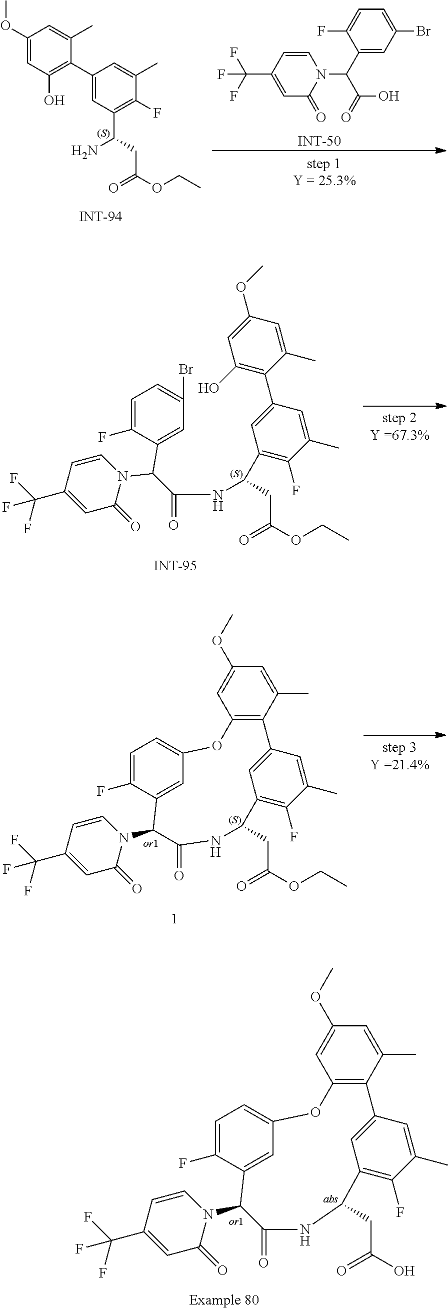

### Step 1: Preparation of ethyl (3S)-3-[2-(5-bromo-2-fluorophenyl)-2-[2-oxo-4-(trifluoromethyl)pyridin-1-yl]acetamido]-3-{4-fluoro-2′-hydroxy-4′-methoxy-5,6′-dimethyl-[1,1′-biphenyl]-3-yl}propanoate

To a stirred mixture of ethyl (3S)-3-amino-3-{4-fluoro-2′-hydroxy-4′-methoxy-5,6′-dimethyl-[1,1′-biphenyl]-3-yl}propanoate (600 mg, 1.66 mmol, 1.00 equiv.) and (5-bromo-2-fluorophenyl)[2-oxo-4-(trifluoromethyl)pyridin-1-yl]acetic acid (654 mg, 1.66 mmol, 1.00 equiv.) in MeCN (15 mL) were added EDCI (636.50 mg, 3.320 mmol, 2 equiv.) and HOBT (448 mg, 3.320 mmol, 2.00 equiv.) in portions at room temperature under nitrogen atmosphere. The resulting mixture was stirred for overnight at room temperature under nitrogen atmosphere. The resulting mixture was purified by reversed-phase flash chromatography (C18 gel; mobile phase, MeCN in water (0.1% NH₃·H₂O), 20% to 40% gradient in 10 min; detector, UV 220 nm) to provide ethyl (3S)-3-[2-(5-bromo-2-fluorophenyl)-2-[2-oxo-4-(trifluoromethyl)pyridin-1-yl]acetamido]-3-{4-fluoro-2′-hydroxy-4′-methoxy-5,6′-dimethyl-[1,1′-biphenyl]-3-yl}propanoate (310 mg, 25.3%, mixture) as a white solid. LC-MS: (ES+H, m/z) [M+H]⁺=737.1. ¹H NMR (300 MHz, DMSO-d₆) δ 9.39 (t, J=9.7 Hz, 1H), 9.13 (d, J=4.0 Hz, 1H), 7.80-7.61 (m, 1H), 7.58-7.14 (m, 3H), 7.04-6.84 (m, 4H), 6.79 (s, 1H), 6.57-6.26 (m, 3H), 5.67-5.55 (m, 1H), 4.14-3.94 (m, 2H), 3.70 (d, J=4.8 Hz, 2H), 2.88-2.66 (m, 1H), 2.25 (dd, J=6.4, 1.9 Hz, 3H), 1.89 (d, J=11.8 Hz, 3H), 1.27-1.10 (m, 1H), 1.08 (t, J=7.2 Hz, 2H), 0.84 (s, 1H).

### Step 2: Preparation of ethyl 2-[(14S,17S)-12,19-difluoro-5-methoxy-3,20-dimethyl-15-oxo-14-[2-oxo-4-(trifluoromethyl)pyridin-1-yl]-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate

To a stirred solution of ethyl (3S)-3-[2-(5-bromo-2-fluorophenyl)-2-[2-oxo-4-(trifluoromethyl)pyridin-1-yl]acetamido]-3-{4-fluoro-2′-hydroxy-4′-methoxy-5,6′-dimethyl-[1,1′-biphenyl]-3-yl}propanoate (100 mg, 0.14 mmol, 1.00 equiv.) and (1R,2R)—N1,N2-dimethylcyclohexane-1,2-diamine (19 mg, 0.14 mmol, 1.00 equiv.) in 1,4-dioxane (2 mL) were added K₂CO₃ (37 mg, 0.27 mmol, 2.00 equiv.) and CuI (12 mg, 0.068 mmol, 0.50 equiv.) in portions at room temperature under nitrogen atmosphere. The resulting mixture was stirred for 2 h at 100° C. under nitrogen atmosphere. The mixture was allowed to cool down to room temperature. The resulting mixture was diluted with EtOAc (100 mL). The combined organic layers were washed with water (2×20 mL), dried over anhydrous Na₂SO₄, filtered, and concentrated. The residue was purified by reversed-phase flash chromatography (C18 gel; mobile phase, MeCN in water (0.1% NH₃·H₂O), 30% to 50% gradient in 10 min; detector, UV 220 nm) to provide ethyl 2-[(14S,17S)-12,19-difluoro-5-methoxy-3,20-dimethyl-15-oxo-14-[2-oxo-4-(trifluoromethyl)pyridin-1-yl]-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (60 mg, 67.3%) as a white solid. LC-MS: (ES+H, m/z) [M+H]⁺=657.20. ¹H NMR (400 MHz, DMSO-d₆) δ 9.21 (d, J=7.4 Hz, 1H), 7.76-7.66 (m, 1H), 7.33 (d, J=7.3 Hz, 1H), 7.26 (t, J=9.2 Hz, 1H), 7.22-7.16 (m, 2H), 6.99 (dd, J=6.8, 2.1 Hz, 1H), 6.79 (d, J=2.5 Hz, 1H), 6.72 (d, J=6.6 Hz, 1H), 6.42 (dd, J=6.4, 2.2 Hz, 1H), 6.33 (dd, J=7.3, 2.3 Hz, 1H), 5.90 (dd, J=6.2, 3.0 Hz, 1H), 5.48 (q, J=7.5 Hz, 1H), 4.06 (dddd, J=17.9, 10.8, 7.1, 3.7 Hz, 2H), 3.85 (s, 3H), 2.83 (dd, J=15.8, 9.0 Hz, 1H), 2.73 (dd, J=15.8, 6.6 Hz, 1H), 2.29 (d, J=1.8 Hz, 3H), 1.92 (s, 3H), 1.12 (t, J=7.1 Hz, 3H).

### Step 3: Preparation of [(14S,17S)-12,19-difluoro-5-methoxy-3,20-dimethyl-15-oxo-14-[2-oxo-4-(trifluoromethyl)pyridin-1-yl]-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid

A solution of ethyl 2-[(14S,17S)-12,19-difluoro-5-methoxy-3,20-dimethyl-15-oxo-14-[2-oxo-4-(trifluoromethyl)pyridin-1-yl]-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (55 mg, 0.084 mmol, 1.00 equiv.) and LiOH·H₂O (10 mg, 0.25 mmol, 3.00 equiv.) in THF (2 mL) and H₂O (0.5 mL) was stirred overnight at room temperature under nitrogen atmosphere. The mixture was acidified to pH 5 with HCl (aq. 1 M). The resulting mixture was concentrated under reduced pressure. The crude product (60 mg) was purified by Prep-HPLC (Column: XBridge Shield RP18 OBD Column, 30*150 mm, 5 μm; Mobile Phase A: water (10 mmol/L NH₄HCO₃+0.05% NH₃·H₂O), Mobile Phase B: MeCN; Flow rate: 60 mL/min; Gradient: 32% B to 34% B in 10 min; Wavelength: 254 nm/220 nm; RT1 (min) 9.1) to afford [(14S,17S)-12,19-difluoro-5-methoxy-3,20-dimethyl-15-oxo-14-[2-oxo-4-(trifluoromethyl)pyridin-1-yl]-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid (11.3 mg, 21.6%) as a white solid. LC-MS: (ES+H, m/z) [M+H]⁺=629.15. ¹H NMR (400 MHz, DMSO-d₆) δ 9.37 (s, 1H), 7.66 (dt, J=7.7, 3.6 Hz, 1H), 7.33 (d, J=7.3 Hz, 1H), 7.23 (t, J=9.1 Hz, 1H), 7.17 (d, J=2.5 Hz, 1H), 6.95 (dd, J=7.0, 2.1 Hz, 1H), 6.85 (s, 1H), 6.81-6.74 (m, 2H), 6.42 (dd, J=6.5, 2.2 Hz, 1H), 6.33 (dd, J=7.3, 2.1 Hz, 1H), 5.94 (dd, J=6.3, 3.0 Hz, 1H), 5.45 (q, J=7.2 Hz, 1H), 3.85 (s, 3H), 2.54 (s, 2H), 2.28 (s, 3H), 1.91 (s, 3H). ¹⁹F NMR (377 MHz, DMSO-d₆) δ −65.44, −121.69, −126.24.

### Example 81

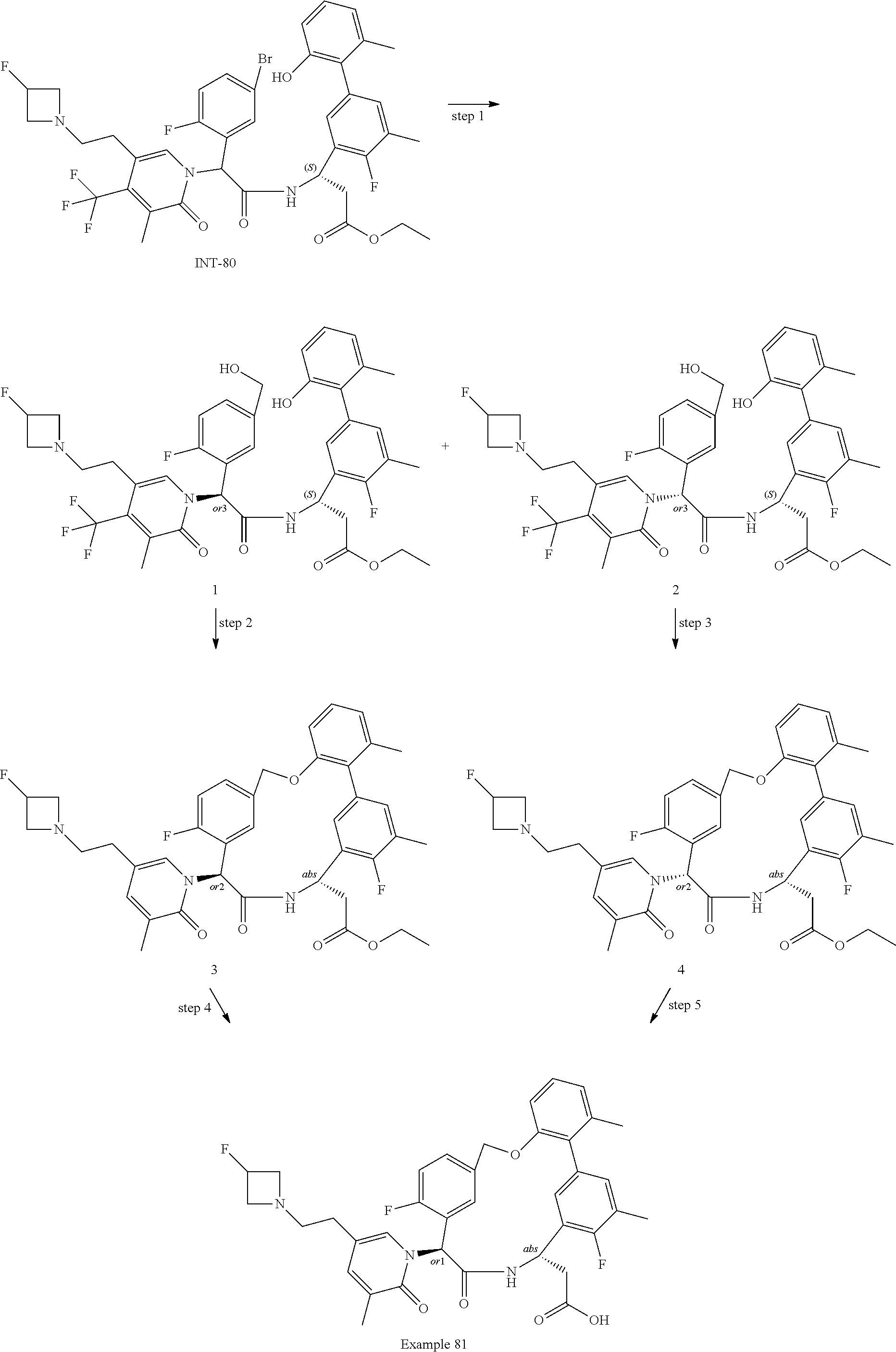

### Step 1: Preparation of ethyl (3S)-3-(4-fluoro-2′-hydroxy-5,6′-dimethyl-[1,1′-biphenyl]-3-yl)-3-(2-(2-fluoro-5-(hydroxymethyl)phenyl)-2-(5-(2-(3-fluoroazetidin-1-yl)ethyl)-3-methyl-2-oxopyridin-1(2H)-yl)acetamido)propanoate

To a stirred solution of ethyl (3S)-3-[2-(5-bromo-2-fluorophenyl)-2-{5-[2-(3-fluoroazetidin-1-yl)ethyl]-3-methyl-2-oxopyridin-1-yl}acetamido]-3-{4-fluoro-2′-hydroxy-5,6′-dimethyl-[1,1′-biphenyl]-3-yl}propanoate (300 mg, 0.39 mmol, 1.00 equiv.) and (tributylstannyl)methanol (641 mg, 1.99 mmol, 5.00 equiv.) in dioxane (50 mL) was added 2nd generation XPhos precatalyst (31 mg, 0.04 mmol, 0.10 equiv.) in portions at room temperature under nitrogen atmosphere. The resulting mixture was stirred for 16 h at 80° C. under nitrogen atmosphere. The resulting mixture was concentrated under reduced pressure. The crude product (220 mg) was purified by Prep-HPLC (Column: XBridge Prep OBD C18 Column, 30*150 mm, 10 μm; Mobile Phase A: water (10 mmol/L NH₄HCO₃+0.05% NH₃H₂O), Mobile Phase B: MeCN; Flow rate: 60 mL/min; Gradient: 37% B to 53% B in 8 min; Wavelength: 254 nm/220 nm; RT1 (min) 9.57/10.40) to afford ethyl (3S)-3-{4-fluoro-2′-hydroxy-5,6′-dimethyl-[1,1′-biphenyl]-3-yl}-3-{2-[2-fluoro-5-(hydroxymethyl)phenyl]-2-{5-[2-(3-fluoroazetidin-1-yl)ethyl]-3-methyl-2-oxopyridin-1-yl}acetamido}propanoate (isomer 1:110 mg, isomer 2: 60 mg) as a white solid. LC-MS: (ES+H, m/z) [M+H]⁺=706.35.

### Step 2: Preparation of ethyl 2-((6S,9S)-14,54-difluoro-6-(5-(2-(3-fluoroazetidin-1-yl)ethyl)-3-methyl-2-oxopyridin-1(2H)-yl)-15,26-dimethyl-7-oxo-3-oxa-8-aza-1,5(1,3),2(1,2)-tribenzenacyclononaphane-9-yl)acetate

To a stirred solution of ethyl (3S)-3-{4-fluoro-2′-hydroxy-5,6′-dimethyl-[1,1′-biphenyl]-3-yl}-3-{2-[2-fluoro-5-(hydroxymethyl)phenyl]-2-{5-[2-(3-fluoroazetidin-1-yl)ethyl]-3-methyl-2-oxopyridin-1-yl}acetamido}propanoate (60 mg, 0.085 mmol, 1.00 equiv.) and CMBP (102 mg, 0.43 mmol, 5.00 equiv.) in toluene (3 mL) was added K₂CO₃ (23 mg, 0.17 mmol, 2.00 equiv.) in portions at room temperature under nitrogen atmosphere. The resulting mixture was stirred for 16 h at 100° C. under nitrogen atmosphere. The mixture was allowed to cool down to room temperature. The resulting mixture was extracted with EtOAc (3×10 mL). The combined organic layers were washed with brine (2×5 mL), dried over anhydrous Na₂SO₄, filtered, and concentrated. The residue was purified by silica gel column chromatography (CH₂Cl₂/MeOH (0 to 10% gradient in 30 min)) to afford ethyl 2-[(15S,18S)-13,20-difluoro-15-{5-[2-(3-fluoroazetidin-1-yl)ethyl]-3-methyl-2-oxopyridin-1-yl}-3,21-dimethyl-16-oxo-8-oxa-17-azatetracyclo[17.3.1.1\hat{ }{10,14}.0\hat{ }{2,7}]tetracosa-1(23),2,4,6,10,12,14(24),19,21-nonaen-18-yl]acetate (85 mg, crude) as a yellow oil. LC-MS: (ES+H, m/z) [M+H]⁺=688.35.

### Step 3: Preparation of ethyl 2-((6S,9S)-14,54-difluoro-6-(5-(2-(3-fluoroazetidin-1-yl)ethyl)-3-methyl-2-oxopyridin-1(2H)-yl)-15,26-dimethyl-7-oxo-3-oxa-8-aza-1,5(1,3),2(1,2)-tribenzenacyclononaphane-9-yl)acetate

To a stirred solution of ethyl (3S)-3-{4-fluoro-2′-hydroxy-5,6′-dimethyl-[1,1′-biphenyl]-3-yl}-3-{2-[2-fluoro-5-(hydroxymethyl)phenyl]-2-{5-[2-(3-fluoroazetidin-1-yl)ethyl]-3-methyl-2-oxopyridin-1-yl}acetamido}propanoate (45 mg, 0.064 mmol, 1.00 equiv., isomer 2) and CMBP (77 mg, 0.32 mmol, 5.00 equiv.) in toluene (3 mL) was added K₂CO₃ (17 mg, 0.13 mmol, 2.00 equiv.) in portions at room temperature under nitrogen atmosphere. The resulting mixture was stirred for 16 h at 100° C. under nitrogen atmosphere. The mixture was allowed to cool down to room temperature. The resulting mixture was extracted with EtOAc (3×10 mL). The combined organic layers were washed with brine (2×5 mL), dried over anhydrous Na₂SO₄, filtered, and concentrated. The residue was purified by silica gel column chromatography (CH₂Cl₂/MeOH (0 to 10% gradient in 30 min)) to afford ethyl 2-[(15S,18S)-13,20-difluoro-15-{5-[2-(3-fluoroazetidin-1-yl)ethyl]-3-methyl-2-oxopyridin-1-yl}-3,21-dimethyl-16-oxo-8-oxa-17-azatetracyclo[17.3.1.1\hat{ }{10,14}.0\hat{ }{2,7}]tetracosa-1(23),2,4,6,10,12,14(24),19,21-nonaen-18-yl]acetate (80 mg, crude, isomer 2) as a yellow oil. LC-MS: (ES+H, m/z) [M+H]⁺=688.35. Preparation of 2-((6S,9S)-14,54-difluoro-6-(5-(2-(3-fluoroazetidin-1-yl)ethyl)-3-methyl-2-oxopyridin-1(2H)-yl)-15,26-dimethyl-7-oxo-3-oxa-8-aza-1,5(1,3),2(1,2)-tribenzenacyclononaphane-9-yl)acetic acid:

Step 3: To a stirred solution of ethyl 2-[(15S,18S)-13,20-difluoro-15-{5-[2-(3-fluoroazetidin-1-yl)ethyl]-3-methyl-2-oxopyridin-1-yl}-3,21-dimethyl-16-oxo-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetate (70 mg, 0.10 mmol, 1.00 equiv.) and LiOH·H₂O (17 mg, 0.41 mmol, 4.00 equiv.) in THF (4 mL) was added H₂O (0.8 mL) dropwise at room temperature. The resulting mixture was stirred for 2 h at room temperature under nitrogen atmosphere. The resulting mixture was concentrated under reduced pressure. The crude product was purified by Prep-HPLC (Column: XBridge Shield RP18 OBD Column 30*150 mm, 5 μm; Mobile Phase A: water (0.1% HCOOH), Mobile Phase B: MeOH; Flow rate: 60 mL/min; Gradient: 36% B to 52% B in 8 min; Wavelength: 254 nm/220 nm; RT1 (min) 9.38).

Step 4: To a stirred solution of ethyl 2-[(15S,18S)-13,20-difluoro-15-{5-[2-(3-fluoroazetidin-1-yl)ethyl]-3-methyl-2-oxopyridin-1-yl}-3,21-dimethyl-16-oxo-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetate (80 mg, 0.11 mmol, 1.00 equiv., isomer 2) and LiOH·H₂O (18 mg, 0.44 mmol, 4.00 equiv.) in THF (4 mL) was added H₂O (0.8 mL) dropwise at room temperature. The resulting mixture was stirred for 2 h at room temperature under nitrogen atmosphere. The resulting mixture was concentrated under reduced pressure. The crude product was purified by Prep-HPLC (Column: XBridge Shield RP18 OBD Column 30*150 mm, 5 μm; Mobile Phase A: water (0.1% HCOOH), Mobile Phase B: MeOH; Flow rate: 60 mL/min; Gradient: 36% B to 52% B in 8 min; Wavelength: 254 nm/220 nm; RT1 (min) 9.57).

The pure fraction was concentrated under reduced pressure and lyophilized to afford [(15S,18S)-13,20-difluoro-15-{5-[2-(3-fluoroazetidin-1-yl)ethyl]-3-methyl-2-oxopyridin-1-yl}-3,21-dimethyl-16-oxo-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetic acid (2.5 mg, 3.63%) as a white solid. LC-MS: (ES+H, m/z) [M+H]⁺=660.20. ¹H NMR (400 MHz, CD₃OD) δ 8.40 (s, 1H), 7.46-7.42 (m, 1H), 7.32 (d, J=9.7 Hz, 2H), 7.20-7.06 (m, 4H), 6.96-6.91 (m, 1H), 6.84-6.78 (m, 2H), 6.70-6.66 (m, 1H), 5.72 (d, J=7.5 Hz, 1H), 5.30 (d, J=13.2 Hz, 1H), 5.08 (s, 1H), 4.98 (d, J=13.2 Hz, 1H), 3.90 (s, 2H), 3.68-3.52 (m, 2H), 2.93 (d, J=7.6 Hz, 2H), 2.68 (d, J=15.4 Hz, 2H), 2.42-2.30 (m, 5H), 2.16 (s, 4H), 1.97 (s, 3H). ⁹F NMR (377 MHz, CD₃OD) δ −118.25, −128.36, −180.98.

### Examples 82 and 83

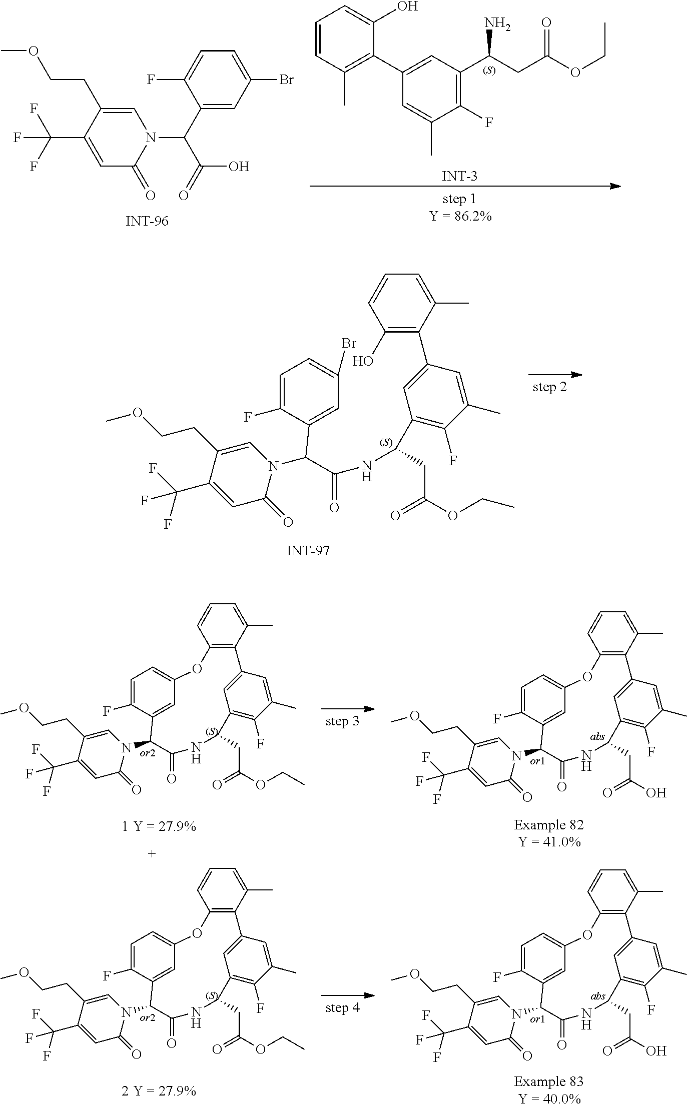

### Step 1: Preparation of ethyl (3S)-3-[2-(5-bromo-2-fluorophenyl)-2-[5-(2-methoxyethyl)-2-oxo-4-(trifluoromethyl)pyridin-1-yl]acetamido]-3-{4-fluoro-2′-hydroxy-5,6′-dimethyl-[1,1′-biphenyl]-3-yl}propanoate

To a stirred solution of (5-bromo-2-fluorophenyl)[5-(2-methoxyethyl)-2-oxo-4-(trifluoromethyl)pyridin-1-yl]acetic acid (185 mg, 0.41 mmol, 1 equiv.) and DIEA (317 mg, 2.45 mmol, 6.00 equiv.) in DMF (4 mL) were added HATU (187 mg, 0.49 mmol, 1.20 equiv.) and ethyl (3S)-3-amino-3-{4-fluoro-2′-hydroxy-5,6′-dimethyl-[1,1′-biphenyl]-3-yl}propanoate (163 mg, 0.49 mmol, 1.20 equiv.) in portions at room temperature. The resulting mixture was stirred overnight at room temperature under air atmosphere. The reaction was quenched by the addition of water/ice (25 mL) at room temperature. The resulting mixture was extracted with EtOAc (3×15 mL). The combined organic layers were washed with brine (1×15 mL), dried over anhydrous Na₂SO₄, filtered, and concentrated. The residue was purified by silica gel column chromatography (petroleum ether/EtOAc (0 to 30% gradient in 15 min) to afford ethyl (3S)-3-[2-(5-bromo-2-fluorophenyl)-2-[5-(2-methoxyethyl)-2-oxo-4-(trifluoromethyl)pyridin-1-yl]acetamido]-3-{4-fluoro-2′-hydroxy-5,6′-dimethyl-[1,1′-biphenyl]-3-yl}propanoate (270 mg, 86.2%, mixture) as a yellow oil. LC-MS: (ES+H, m/z) [M+H]⁺=765.2.

### Step 2: Preparation of ethyl 2-[(14R*,17S)-12,19-difluoro-14-[5-(2-methoxyethyl)-2-oxo-4-(trifluoromethyl)pyridin-1-yl]-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate and ethyl 2-[(14S,17S)-12,19-difluoro-14-[5-(2-methoxyethyl)-2-oxo-4-(trifluoromethyl)pyridin-1-yl]-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate

To a stirred solution of ethyl (3S)-3-[2-(5-bromo-2-fluorophenyl)-2-[5-(2-methoxyethyl)-2-oxo-4-(trifluoromethyl)pyridin-1-yl]acetamido]-3-{4-fluoro-2′-hydroxy-5,6′-dimethyl-[1,1′-biphenyl]-3-yl}propanoate (200 mg, 0.26 mmol, 1.00 equiv.) and (1R,2S)—N1,N2-dimethylcyclohexane-1,2-diamine (37 mg, 0.26 mmol, 1.00 equiv.) in dioxane (4 mL) were added K₂CO₃ (72 mg, 0.52 mmol, 2.00 equiv.) and CuI (25 mg, 0.13 mmol, 0.50 equiv.) in portions at room temperature. The resulting mixture was stirred overnight at 100° C. under nitrogen atmosphere. The resulting mixture was concentrated under reduced pressure. The residue was purified by Prep-TLC (CH₂Cl₂/MeOH 15:1, Rf=0.5) to afford ethyl 2-[(14R*,17S)-12,19-difluoro-14-[5-(2-methoxyethyl)-2-oxo-4-(trifluoromethyl)pyridin-1-yl]-3,20-dimethyl-15-oxo-8-oxa-16-azatetracyclo[16.3.1.1\hat{ }{9,13}.0\hat{ }{2,7}]tricosa-1(22),2(7),3,5,9,11,13(23), 18,20-nonaen-17-yl]acetate (50 mg, 27.9%) and ethyl 2-[(14S,17S)-12,19-difluoro-14-[5-(2-methoxyethyl)-2-oxo-4-(trifluoromethyl)pyridin-1-yl]-3,20-dimethyl-15-oxo-8-oxa-16-azatetracyclo [16.3.1.1\hat{ }{9,13}.0\hat{ }{2,7}]tricosa-1(22),2(7),3,5,9,11,13(23),18,20-nonaen-17-yl]acetate (50 mg, 27.9%) as an off-white solid. LC-MS: (ES+H, m/z) [M+H]⁺=685.3.

### Step 3: Preparation of [(14R*,17S)-12,19-difluoro-14-[5-(2-methoxyethyl)-2-oxo-4-(trifluoromethyl)pyridin-1-yl]-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid

A solution of ethyl 2-[(14R*,17S)-12,19-difluoro-14-[5-(2-methoxyethyl)-2-oxo-4-(trifluoromethyl)pyridin-1-yl]-3,20-dimethyl-15-oxo-8-oxa-16-azatetracyclo[16.3.1.1\hat{ }{9,13}.0\hat{ }{2,7}]tricosa-1(22),2(7),3,5,9,11,13 (23),18,20-nonaen-17-yl]acetate (50 mg, 0.07 mmol, 1.00 equiv.) and LiOH·H₂O (6 mg, 0.15 mmol, 2.00 equiv.) in THF (1.2 mL) and H₂O (0.4 mL) was stirred overnight at room temperature and then was concentrated. The crude product (53 mg) was purified by Prep-HPLC (Column: XBridge Shield RP18 OBD Column, 30*150 mm, 5 μm; Mobile Phase A: water (10 mmol/L NH₄HCO₃), Mobile Phase B: MeCN; Flow rate: 60 mL/min; Gradient: 28% B to 48% B in 10 min; Wavelength: 254 nm/220 nm; RT1 (min) 8.5) to afford [(14R*,17S)-12,19-difluoro-14-[5-(2-methoxyethyl)-2-oxo-4-(trifluoromethyl) pyridin-1-yl]-3,20-dimethyl-15-oxo-8-oxa-16-azatetracyclo[16.3.1.1\hat{ }{9,13}.0\hat{ }{2,7}]tricosa-1(22),2(7),3,5, 9,11,13(23),18,20-nonaen-17-yl]acetic acid (19.7 mg, 41.0%) as a white solid.

Example 82: LC-MS: (ES+H, m/z) [M+H]⁺=657.15. ¹H NMR (400 MHz, CD₃OD) δ 7.58-7.50 (m, 1H), 7.43 (d, J=8.0 Hz, 1H), 7.33 (t, J=7.9 Hz, 1H), 7.24 (s, 1H), 7.21-7.12 (m, 2H), 6.97 (d, J=6.9, 2.1 Hz, 1H), 6.85 (s, 1H), 6.79 (s, 1H), 6.46 (dd, J=6.3, 2.2 Hz, 1H), 6.05 (dd, J=6.1, 3.0 Hz, 1H), 5.70 (t, J=7.5 Hz, 1H), 3.46-3.36 (m, 1H), 3.28 (s, 1H), 3.02 (s, 3H), 2.82-2.72 (m, 1H), 2.72-2.59 (m, 3H), 2.35 (d, J=2.0 Hz, 3H), 1.99 (s, 3H). ¹⁹F NMR (377 MHz, CD₃OD) δ −65.30, −123.38, −126.75.

### Step 4: Preparation of [(14S,17S)-12,19-difluoro-14-[5-(2-methoxyethyl)-2-oxo-4-(trifluoromethyl)pyridin-1-yl]-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid

A solution of ethyl 2-[(14S,17S)-12,19-difluoro-14-[5-(2-methoxyethyl)-2-oxo-4-(trifluoromethyl)pyridin-1-yl]-3,20-dimethyl-15-oxo-8-oxa-16-azatetracyclo[16.3.1.1\hat{ }{9,13}.0\hat{ }{2,7}]tricosa-1(22),2(7),3,5,9,11,13 (23),18,20-nonaen-17-yl]acetate (50 mg, 0.07 mmol, 1.00 equiv.) and LiOH·H₂O (6 mg, 0.15 mmol, 2.00 equiv.) in THF (1.2 mL) and H₂O (0.4 mL) was stirred overnight at room temperature and then was concentrated. The crude product (50 mg) was purified by Prep-HPLC (Column: XBridge Shield RP18 OBD Column, 30*150 mm, 5 μm; Mobile Phase A: water (10 mmol/L NH₄HCO₃+0.05% NH₃·H₂O), Mobile Phase B: MeCN; Flow rate: 60 mL/min; Gradient: 25% B to 45% B in 10 min; Wavelength: 254 nm/220 nm; RT1 (min) 8) to afford [(14S,17S)-12,19-difluoro-14-[5-(2-methoxyethyl)-2-oxo-4-(trifluoromethyl)pyridin-1-yl]-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid (19.2 mg, 40.0%) as a white solid.

Example 83: LC-MS: (ES+H, m/z) [M+H]⁺=657.15. ¹H NMR (400 MHz, CD₃OD) δ 7.70 (d, J=16.1 Hz, 1H), 7.29 (t, J=7.9 Hz, 2H), 7.22 (s, 1H), 7.19-7.07 (m, 3H), 6.98-6.87 (m, 3H), 6.81 (d, J=17.5 Hz, 2H), 6.45 (d, J=16.2 Hz, 1H), 3.49-3.36 (m, 1H), 3.27 (s, 2H), 2.98 (s, 3H), 2.63 (t, J=5.8 Hz, 2H), 2.27 (d, J=2.2 Hz, 3H), 2.15 (s, 3H). ¹⁹F NMR (377 MHz, CD₃OD) δ −65.27.

### Example 84

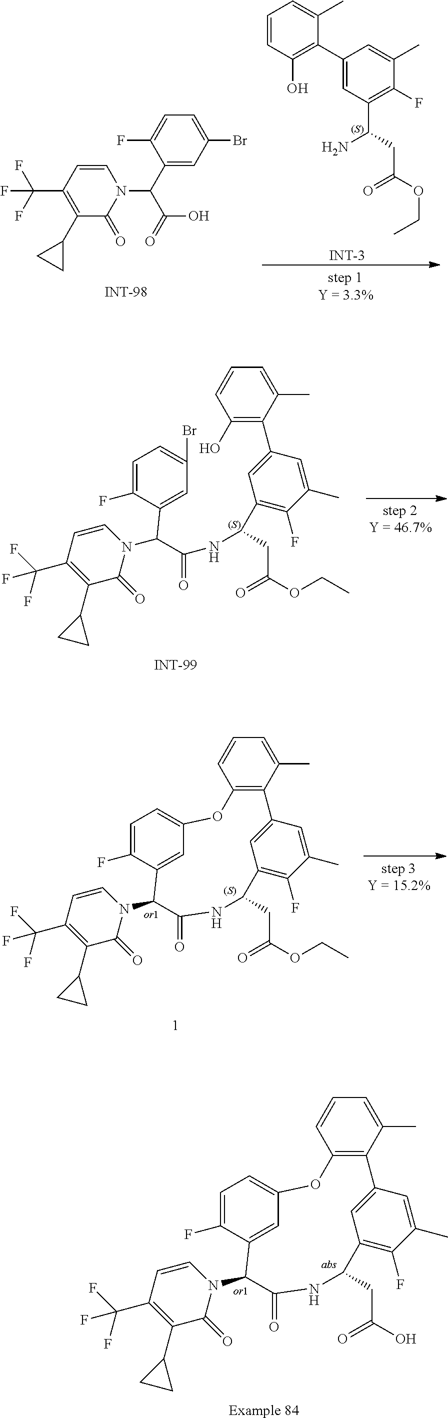

### Step 1: Preparation of ethyl (3S)-3-(2-(5-bromo-2-fluorophenyl)-2-(3-cyclopropyl-2-oxo-4-(trifluoromethyl)pyridin-1(2H)-yl)acetamido)-3-(4-fluoro-2′-hydroxy-5,6′-dimethyl-[1,1′-biphenyl]-3-yl)propanoate

To a stirred solution of (5-bromo-2-fluorophenyl)[3-cyclopropyl-2-oxo-4-(trifluoromethyl)pyridin-1-yl]acetic acid (1.11 g, 2.55 mmol, 1.00 equiv.) and ethyl (3S)-3-amino-3-{4-fluoro-2′-hydroxy-5,6′-dimethyl-[1,1′-biphenyl]-3-yl}propanoate (0.93 g, 2.81 mmol, 1.10 equiv.) in MeCN (8 mL) was added NEt₃ (0.52 g, 5.14 mmol, 2.01 equiv.) in portions at 40° C. under nitrogen atmosphere. To the above mixture was added EDCI (1.47 g, 7.66 mmol, 3.00 equiv.) and HOBT (0.69 g, 5.11 mmol, 2.00 equiv.) in MeCN (4 mL) in portions at room temperature. The resulting mixture was stirred overnight at 50° C. under nitrogen atmosphere. The mixture was allowed to cool down to room temperature. The resulting mixture was concentrated under reduced pressure. The resulting mixture was diluted with CH₂Cl₂ (100 mL). The combined organic layers were washed with brine (3×30 mL), dried over anhydrous Na₂SO₄, filtered, and concentrated. The residue was purified by silica gel column chromatography (petroleum ether/EtOAc (20% to 30% gradient in 10 min) to afford ethyl (3S)-3-(2-(5-bromo-2-fluorophenyl)-2-(3-cyclopropyl-2-oxo-4-(trifluoromethyl)pyridin-1(2H)-yl)acetamido)-3-(4-fluoro-2′-hydroxy-5,6′-dimethyl-[1,1′-biphenyl]-3-yl)propanoate (170 mg, 3.3%, mixture) as a yellow solid. LC-MS: (ES+H, m/z) [M+H]⁺=747.05.

### Step 2: Preparation of ethyl 2-[(14R*,17S)-14-[3-cyclopropyl-2-oxo-4-(trifluoromethyl)pyridin-1-yl]-12,19-difluoro-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate

To a stirred solution of ethyl (3S)-3-[2-(5-bromo-2-fluorophenyl)-2-[3-cyclopropyl-2-oxo-4-(trifluoromethyl)pyridin-1-yl]acetamido]-3-{4-fluoro-2′-hydroxy-5,6′-dimethyl-[1,1′-biphenyl]-3-yl}propanoate (65 mg, 0.09 mmol, 1.00 equiv.) and (1R,2R)—N1,N2-dimethylcyclohexane-1,2-diamine (12 mg, 0.09 mmol, 1.00 equiv.) in dioxane (2 mL) were added K₂CO₃ (24 mg, 0.17 mmol, 2.00 equiv.) and CuI (8 mg, 0.04 mmol, 0.50 equiv.) in portions at room temperature under nitrogen atmosphere. The resulting mixture was stirred overnight at 100° C. under nitrogen atmosphere. The mixture was allowed to cool down to room temperature. The resulting mixture was concentrated under reduced pressure. The residue was purified by reversed-phase flash chromatography (C18 gel; mobile phase, MeCN in water (0.1% NH₃·H₂O), 70% to 80% gradient in 10 min; detector, UV 254 nm) to afford ethyl 2-[(14R*,17S)-14-[3-cyclopropyl-2-oxo-4-(trifluoromethyl)pyridin-1-yl]-12,19-difluoro-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (48 mg, 46.7%) as a light yellow solid. LC-MS: (ES+H, m/z) [M+H]⁺=667.20. 1H NMR (400 MHz, CD₃OD) δ 7.52 (dt, J=9.2, 3.7 Hz, 1H), 7.43 (d, J=8.0 Hz, 1H), 7.34 (d, J=7.8 Hz, 1H), 7.14 (dd, J=10.0, 8.1 Hz, 2H), 7.04 (d, J=7.5 Hz, 1H), 6.97 (d, J=6.3 Hz, 1H), 6.65 (d, J=3.1 Hz, 1H), 6.42 (dd, J=6.3, 2.2 Hz, 1H), 6.37 (d, J=7.5 Hz, 1H), 6.02 (dd, J=6.2, 3.0 Hz, 1H), 5.68 (t, J=7.9 Hz, 1H), 4.11 (p, J=7.1 Hz, 2H), 2.86 (dd, J=15.4, 8.1 Hz, 1H), 2.72 (dd, J=15.3, 7.7 Hz, 1H), 2.33 (s, 3H), 1.99 (s, 3H), 1.83 (s, 1H), 1.19 (t, J=7.1 Hz, 3H), 0.91-0.87 (m, 4H). ¹⁹F NMR (377 MHz, CD₃OD) δ −62.19, −123.54, −125.58.

### Step 3: Preparation of [(14R*,17S)-14-[3-cyclopropyl-2-oxo-4-(trifluoromethyl)pyridin-1-yl]-12,19-difluoro-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid

A solution of ethyl 2-[(14R*,17S)-14-[3-cyclopropyl-2-oxo-4-(trifluoromethyl)pyridin-1-yl]-12,19-difluoro-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (43 mg, 0.07 mmol, 1.00 equiv.) and LiOH·H₂O (11 mg, 0.26 mmol, 4.00 equiv.) in THF (1 mL) and H₂O (0.25 mL) was stirred for 2 h at room temperature under nitrogen atmosphere. The mixture was acidified to pH 5 with 1 M HCl (aq.). The resulting mixture was concentrated under reduced pressure. The crude product was purified by Prep-HPLC (Column: Xbridge Phenyl OBD Column, 19*150 mm, 5 μm; Mobile Phase A: water (10 mmol/L NH₄HCO₃+0.05% NH₃H₂O, Mobile Phase B: CH₃CN; Flow rate: 60 mL/min; Gradient: 30% B to 45% B in 15 min; Wavelength: 254 nm/220 nm; RT1 (min) 9.02) to afford [(14R*,17S)-14-[3-cyclopropyl-2-oxo-4-(trifluoromethyl)pyridin-1-yl]-12,19-difluoro-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid (6.3 mg, 15.2%) as a white solid. LC-MS: (ES+H, m/z) [M+H]⁺=639.10. ¹H NMR (400 MHz, DMSO-d₆) δ 9.19 (d, J=7.1 Hz, 1H), 7.49 (t, J=5.9 Hz, 2H), 7.30 (t, J=7.9 Hz, 1H), 7.22-7.08 (m, 2H), 7.00 (d, J=7.5 Hz, 1H), 6.91 (d, J=6.6 Hz, 1H), 6.56 (s, 1H), 6.36 (d, J=6.2 Hz, 1H), 6.23 (d, J=7.4 Hz, 1H), 5.83 (dd, J=6.2, 2.9 Hz, 1H), 5.38 (d, J=7.2 Hz, 1H), 2.22 (s, 2H), 2.00 (s, 3H), 1.88 (s, 2H), 1.79-1.69 (m, 1H), 1.31 (ddt, J=27.8, 12.1, 4.9 Hz, 2H), 1.17 (s, 1H), 0.78 (qd, J=9.0, 8.2, 4.4 Hz, 2H). ¹⁹F NMR (377 MHz, DMSO-d₆) δ −59.77, −121.73, −125.95.

### Example 85

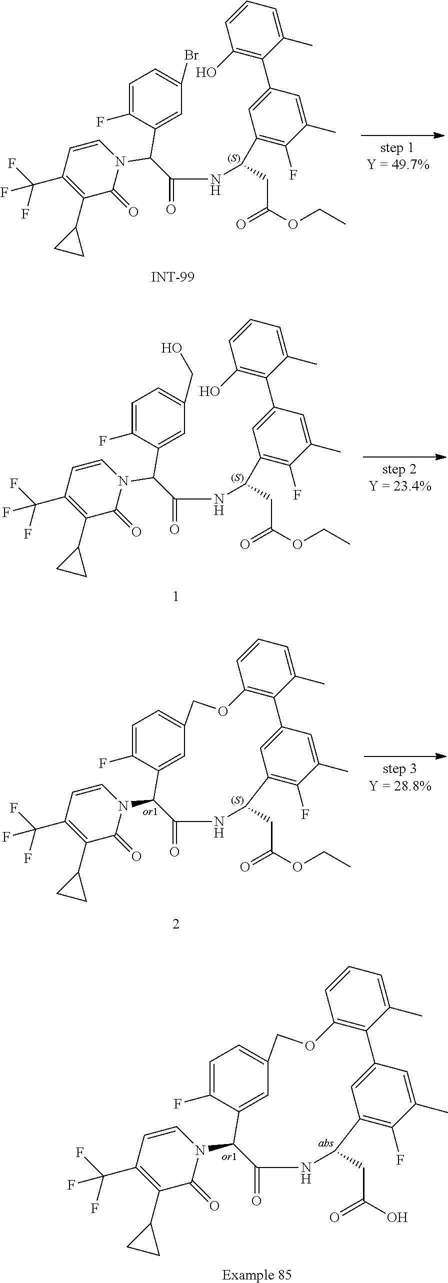

### Step 1: Preparation of ethyl (3S)-3-{2-[3-cyclopropyl-2-oxo-4-(trifluoromethyl)pyridin-1-yl]-2-[2-fluoro-5-(hydroxymethyl)phenyl]acetamido}-3-{4-fluoro-2′-hydroxy-5,6′-dimethyl-[1,1′-biphenyl]-3-yl}propanoate

A mixture of ethyl (3S)-3-[2-(5-bromo-2-fluorophenyl)-2-[3-cyclopropyl-2-oxo-4-(trifluoromethyl)pyridin-1-yl]acetamido]-3-{4-fluoro-2′-hydroxy-5,6′-dimethyl-[1,1′-biphenyl]-3-yl}propanoate (86 mg, 0.11 mmol, 1.00 equiv.), (tributylstannyl)methanol (73 mg, 0.23 mmol, 2.00 equiv.) and Xphos Pd G2 (9 mg, 0.01 mmol, 0.10 equiv.) in 1,4-dioxane (2 mL) was stirred for 2 h at 90° C. under nitrogen atmosphere. The resulting mixture was concentrated under reduced pressure. The residue was purified by reversed-phase flash chromatography (C18 gel; mobile phase, MeCN in water (0.1% NH₃·H₂O), 50% to 65% gradient in 10 min; detector, UV 254 nm) to provide ethyl (3S)-3-{2-[3-cyclopropyl-2-oxo-4-(trifluoromethyl)pyridin-1-yl]-2-[2-fluoro-5-(hydroxymethyl)phenyl]acetamido}-3-{4-fluoro-2′-hydroxy-5,6′-dimethyl-[1,1′-biphenyl]-3-yl}propanoate (40 mg, 49.7%, mixture) as an off-white solid. LC-MS: (ES+H, m/z) [M+H]⁺=699.25.

### Step 2: Preparation of ethyl 2-[(15S,18S)-15-[3-cyclopropyl-2-oxo-4-(trifluoromethyl)pyridin-1-yl]-13,20-difluoro-3,21-dimethyl-16-oxo-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetate

A mixture of ethyl (3S)-3-{2-[3-cyclopropyl-2-oxo-4-(trifluoromethyl)pyridin-1-yl]-2-[2-fluoro-5-(hydroxymethyl)phenyl]acetamido}-3-{4-fluoro-2′-hydroxy-5,6′-dimethyl-[1,1′-biphenyl]-3-yl}propanoate (35 mg, 0.05 mmol, 1.00 equiv.) and CMBP (60 mg, 0.25 mmol, 5.00 equiv.) in toluene (1 mL) was stirred for 1 h at 100° C. under nitrogen atmosphere. The mixture was allowed to cool down to room temperature. The resulting mixture was concentrated under reduced pressure. The residue was purified by reversed-phase flash chromatography (C18 gel; mobile phase, MeCN in water (0.1% NH₃·H₂O), 60% to 80% gradient in 20 min; detector, UV 254 nm) to provide ethyl 2-[(15S,18S)-15-[3-cyclopropyl-2-oxo-4-(trifluoromethyl)pyridin-1-yl]-13,20-difluoro-3,21-dimethyl-16-oxo-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetate (8 mg, 23.4%) as a white solid. LC-MS: (ES+H, m/z) [M+H]⁻=681.3.

### Step 3: Preparation of [(15R*,18S)-15-[3-cyclopropyl-2-oxo-4-(trifluoromethyl)pyridin-1-yl]-13,20-difluoro-3,21-dimethyl-16-oxo-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetic acid

A mixture of ethyl 2-[(15S,18S)-15-[3-cyclopropyl-2-oxo-4-(trifluoromethyl)pyridin-1-yl]-13,20-difluoro-3,21-dimethyl-16-oxo-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetate (8 mg, 0.012 mmol, 1.00 equiv.) and LiOH·H₂O (1.48 mg, 0.036 mmol, 3.00 equiv.) in THF (0.5 mL) and H₂O (0.1 mL) was stirred for 2 h at room temperature under nitrogen atmosphere and then concentrated. The crude product (10 mg) was purified by Prep-HPLC (Column: Xselect CSH C18 OBD Column 30*150 mm, 5 μm; Mobile Phase A: water (0.1% HCOOH), Mobile Phase B: MeCN; Flow rate: 60 mL/min; Gradient: 55% B to 75% B in 10 min; Wavelength: 254 nm/220 nm; RT1 (min) 9.07) to afford [(15R*,18S)-15-[3-cyclopropyl-2-oxo-4-(trifluoromethyl)pyridin-1-yl]-13,20-difluoro-3,21-dimethyl-16-oxo-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetic acid (2.3 mg, 28.79%) as a white solid. LC-MS: (ES−H, m/z) [M−H]⁻=651.15. ¹H NMR (400 MHz, CD₃OD) δ 7.45 (ddd, J=7.7, 5.0, 2.1 Hz, 1H), 7.26 (d, J=7.4 Hz, 1H), 7.20-7.04 (m, 3H), 7.00 (d, J=7.5 Hz, 1H), 6.94 (d, J=8.3 Hz, 2H), 6.81 (d, J=7.2 Hz, 1H), 6.61 (d, J=6.7 Hz, 1H), 6.31 (d, J=7.5 Hz, 1H), 5.81-5.69 (m, 1H), 5.29 (d, J=13.2 Hz, 1H), 4.96 (d, J=13.1 Hz, 1H), 2.78-2.65 (m, 2H), 2.31 (s, 3H), 1.96 (s, 3H), 1.88-1.76 (m, 1H), 1.34-1.27 (m, 2H), 0.94-0.88 (m, 2H). ¹⁹F NMR (377 MHz, CD₃OD) δ −62.18, −118.50, −128.32.

### Example 86

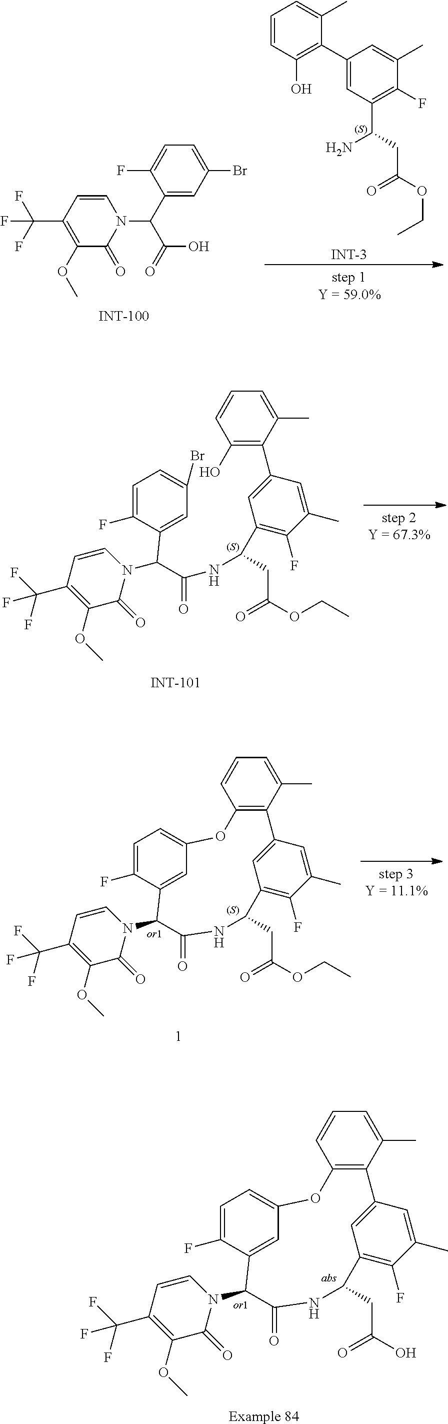

### Step 1: Preparation of ethyl (3S)-3-[2-(5-bromo-2-fluorophenyl)-2-[3-methoxy-2-oxo-4-(trifluoromethyl)pyridin-1-yl]acetamido]-3-{4-fluoro-2′-hydroxy-5,6′-dimethyl-[1,1′-biphenyl]-3-yl}propanoate

Section A: To a stirred solution of ethyl (3S)-3-amino-3-{4-fluoro-2′-hydroxy-5,6′-dimethyl-[1,1′-biphenyl]-3-yl}propanoate (660 mg, 1.99 mmol, 1.30 equiv.) in CH₃CN (5 mL) was added TEA (620 mg, 6.12 mmol, 4.00 equiv.) in portions at room temperature under nitrogen atmosphere. The resulting mixture was stirred for 1 h at 40° C. under nitrogen atmosphere. The resulting mixture was concentrated under vacuum.

To a stirred solution of (5-bromo-2-fluorophenyl)[3-methoxy-2-oxo-4-(trifluoromethyl)pyridin-1-yl]acetic acid (600 mg, 1.53 mmol, 1.00 equiv.) in CH₃CN (12 mL) were added EDCI (440 mg, 2.29 mmol, 1.50 equiv.) and HOBT (269 mg, 1.99 mmol, 1.30 equiv.) in portions at room temperature under nitrogen atmosphere. The resulting mixture was stirred for 1 h at 40° C. under nitrogen atmosphere. To the above mixture was added Section A (in CH₃CN, 3 mL) dropwise at room temperature. The resulting mixture was stirred for an additional 5 h at room temperature. The resulting mixture was diluted with water (100 mL). The above mixture was extracted with EtOAc (3×200 mL). The combined organic layers were dried over anhydrous Na₂SO₄, filtered, and concentrated. The residue was purified by reversed-phase flash chromatography (C18 gel; mobile phase, CH₃CN in water (10 mmol/L NH₄HCO₃), 0% to 70% gradient in 30 min; detector, UV 254 nm) to afford ethyl (3S)-3-[2-(5-bromo-2-fluorophenyl)-2-[3-methoxy-2-oxo-4-(trifluoromethyl)pyridin-1-yl]acetamido]-3-{4-fluoro-2′-hydroxy-5,6′-dimethyl-[1,1′-biphenyl]-3-yl}propanoate (600 mg, 53.0%, mixture) as an off-white solid.

LC-MS: (ES+H, m/z) [M+H]⁺=737.05. ¹H NMR (400 MHz, DMSO-d₆) δ 9.39 (dd, J=25.4, 8.3 Hz, 1H), 9.10 (d, J=1.9 Hz, 1H), 7.78-7.64 (m, 1H), 7.35-7.23 (m, 1H), 7.12 (dd, J=19.6, 7.5 Hz, 1H), 7.07-6.94 (m, 3H), 6.85 (d, J=4.2 Hz, 1H), 6.78-6.66 (m, 2H), 6.31 (dd, J=21.3, 7.5 Hz, 1H), 5.64-5.61 (m, 1H), 4.10-4.00 (m, 2H), 3.88 (d, J=34.5 Hz, 3H), 2.95-2.74 (m, 2H), 2.31-2.21 (m, 3H), 1.91 (d, J=17.9 Hz, 3H), 1.10 (q, J=7.3 Hz, 3H). ¹⁹F NMR (400 MHz, DMSO-d₆) δ −62.01, −117.00, −126.32.

### Step 2: Preparation of ethyl 2-[(14S,17S)-12,19-difluoro-14-[3-methoxy-2-oxo-4-(trifluoromethyl)pyridin-1-yl]-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate

To a stirred solution of ethyl (3S)-3-[2-(5-bromo-2-fluorophenyl)-2-[3-methoxy-2-oxo-4-(trifluoromethyl)pyridin-1-yl]acetamido]-3-{4-fluoro-2′-hydroxy-5,6′-dimethyl-[1,1′-biphenyl]-3-yl}propanoate (190 mg, 0.27 mmol, 1.00 equiv.) and (1S,2S)—N1,N2-dimethylcyclohexane-1,2-diamine (38 mg, 0.27 mmol, 1.00 equiv.) in 1,4-dioxane (20 mL) were added K₂CO₃ (74 mg, 0.54 mmol, 2.00 equiv.) and CuI (25 mg, 0.13 mmol, 0.50 equiv.) in portions at room temperature under nitrogen atmosphere. The resulting mixture was stirred overnight at 100° C. under nitrogen atmosphere. The resulting mixture was washed with 2×100 mL of EtOAc. The resulting mixture was concentrated under vacuum. The residue was purified by reversed-phase flash chromatography (C18 gel; mobile phase, CH₃CN in water (0.1% NH₃·H₂O), 40% to 70% gradient in 15 min; detector, UV 254 nm) to afford ethyl 2-[(14S,17S)-12,19-difluoro-14-[3-methoxy-2-oxo-4-(trifluoromethyl)pyridin-1-yl]-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (130 mg, 67.3%, mixture) as a white solid. LC-MS: (ES+H, m/z) [M+H]⁺=657.15. 1H NMR (400 MHz, DMSO-d6) δ 9.20 (d, J=7.4 Hz, 1H), 7.63-7.57 (m, 2H), 7.38 (t, J=7.9 Hz, 1H), 7.27 (t, J=9.1 Hz, 1H), 7.20 (d, J=7.6 Hz, 1H), 7.03 (d, J=7.4 Hz, 2H), 6.71 (s, 1H), 6.43 (dd, J=6.4, 2.2 Hz, 1H), 6.25 (d, J=7.5 Hz, 1H), 5.89 (dd, J=6.2, 3.0 Hz, 1H), 5.52-5.46 (m, 1H), 4.14-4.00 (m, 2H), 3.94 (s, 3H), 2.89-2.71 (m, 2H), 2.30 (d, J=1.8 Hz, 3H), 1.95 (s, 3H), 1.13 (t, J=7.1 Hz, 3H).

### Step 3: Preparation of [(14S,17S)-12,19-difluoro-14-[3-methoxy-2-oxo-4-(trifluoromethyl)pyridin-1-yl]-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid

To a stirred mixture of ethyl 2-[(14S,17S)-12,19-difluoro-14-[3-methoxy-2-oxo-4-(trifluoromethyl)pyridin-1-yl]-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (120 mg, 0.18 mmol, 1.00 equiv.) in THF (5 mL) was added LiOH·H₂O (15 mg, 0.36 mmol, 2.00 equiv.) in water (2.5 mL) at room temperature under nitrogen atmosphere. The resulting mixture was stirred for 1 h at room temperature under nitrogen atmosphere. The resulting mixture was diluted with water (2.5 mL). The mixture was acidified to pH 5 with 1 M HCl (aq.). The resulting mixture was concentrated under vacuum. The crude product (100 mg) was purified by Prep-HPLC (Column: XBridge Shield RP18 OBD Column, 30*150 mm, 5 μm; Mobile Phase A: water (10 mmol/L NH₄HCO₃+0.05% NH₃·H₂O), Mobile Phase B: CH₃CN; Flow rate: 60 mL/min; Gradient: 35% B to 50% B in 10 min; Wavelength: 254 nm/220 nm; RT1 (min) 8.2) to afford [(14S,17S)-12,19-difluoro-14-[3-methoxy-2-oxo-4-(trifluoromethyl)pyridin-1-yl]-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid (12.8 mg, 11.1%) as a white solid. LC-MS: (ES+H, m/z) [M+H]⁺=629.15. ¹H NMR (400 MHz, CD₃OD) δ 7.54-7.50 (m, 1H), 7.43 (d, J=8.1 Hz, 1H), 7.33 (t, J=7.8 Hz, 1H), 7.17-7.12 (m, 2H), 6.98 (t, J=7.0 Hz, 2H), 6.78 (s, 1H), 6.44 (dd, J=6.3, 2.2 Hz, 1H), 6.31 (d, J=7.4 Hz, 1H), 6.03 (dd, J=6.2, 3.0 Hz, 1H), 5.70 (t, J=7.6 Hz, 1H), 3.98 (s, 3H), 2.86-2.68 (m, 2H), 2.34 (d, J=2.0 Hz, 3H), 1.99 (s, 3H). ¹⁹F NMR (400 MHz, CD₃OD) δ −64.61, −123.49, −126.70.

### Examples 87 and 88

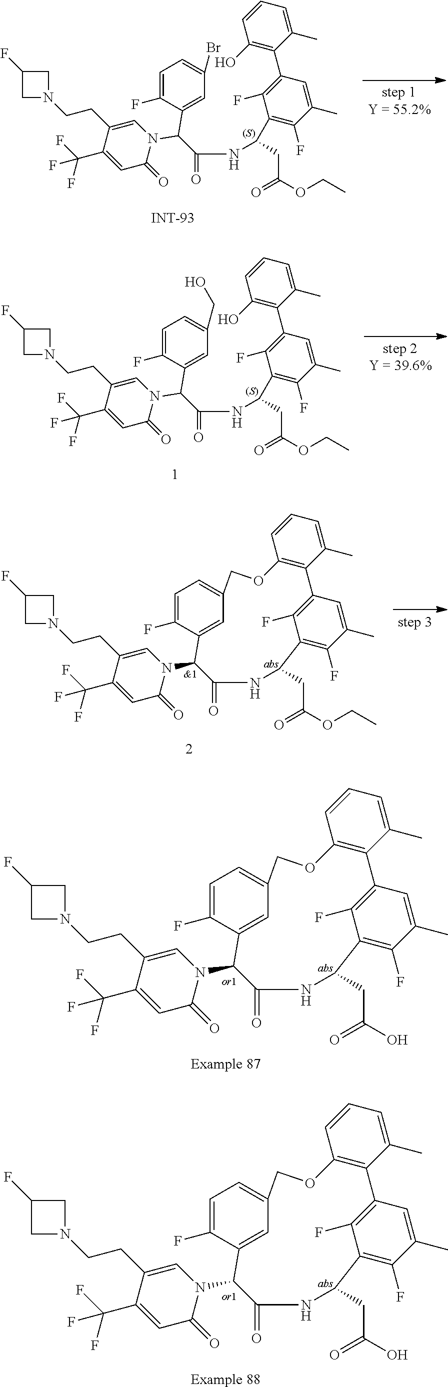

### Step 1: Preparation of ethyl (3S)-3-{2,4-difluoro-2′-hydroxy-5,6′-dimethyl-[1,1′-biphenyl]-3-yl}-3-{2-[2-fluoro-5-(hydroxymethyl)phenyl]-2-{5-[2-(3-fluoroazetidin-1-yl)ethyl]-2-oxo-4-(trifluoromethyl)pyridin-1-yl}acetamido}propanoate

To a stirred mixture of ethyl (3S)-3-[2-(5-bromo-2-fluorophenyl)-2-{5-[2-(3-fluoroazetidin-1-yl)ethyl]-2-oxo-4-(trifluoromethyl)pyridin-1-yl}acetamido]-3-{2,4-difluoro-2′-hydroxy-5,6′-dimethyl-[1,1′-biphenyl]-3-yl}propanoate (500 mg, 0.60 mmol, 1.00 equiv.) and (tributylstannyl)methanol (388 mg, 1.21 mmol, 2.00 equiv.) in 1,4-dioxane (10 mL) was added XPhos-PdCl-2nd G (45 mg, 0.06 mmol, 0.10 equiv.) in portions at room temperature under nitrogen atmosphere. The resulting mixture was stirred overnight at 80° C. under nitrogen atmosphere. The mixture was allowed to cool down to room temperature. The resulting mixture was filtered, and the filter cake was washed with EtOAc. The filtrate was concentrated under reduced pressure. The residue was purified by reversed-phase flash chromatography (C18 gel; mobile phase, MeCN in water (0.1% NH₃·H₂O), 20% to 60% gradient in 25 min; detector, UV 220 nm) to afford ethyl (3S)-3-{2,4-difluoro-2′-hydroxy-5,6′-dimethyl-[1,1′-biphenyl]-3-yl}-3-{2-[2-fluoro-5-(hydroxymethyl)phenyl]-2-{5-[2-(3-fluoroazetidin-1-yl)ethyl]-2-oxo-4-(trifluoromethyl)pyridin-1-yl}acetamido}propanoate (260 mg, 55.2%, mixture) as a white solid. LC-MS: (ES+H, m/z): [M+H]⁺=778.3. ¹H NMR (400 MHz, DMSO-d₆) δ 9.44-9.32 (m, 1H), 9.31-9.23 (m, 1H), 7.44 (dtt, J=10.3, 5.1, 2.2 Hz, 1H), 7.29-7.19 (m, 1H), 7.18-7.02 (m, 3H), 6.89 (dd, J=8.0, 6.1 Hz, 1H), 6.83-6.67 (m, 3H), 5.71 (ddt, J=31.1, 15.4, 7.7 Hz, 1H), 5.39-5.13 (m, 1H), 5.00-4.74 (m, 1H), 4.63-4.20 (m, 2H), 4.08-3.89 (m, 2H), 3.31-3.20 (m, 2H), 3.05-2.76 (m, 4H), 2.46-2.25 (m, 4H), 2.23-2.14 (m, 3H), 2.07 (s, 2H), 1.94 (d, J=18.0 Hz, 2H), 1.18-0.97 (m, 3H). ¹⁹F NMR (377 MHz, DMSO-d₆) δ −62.63, −116.53, −117.87, −118.35, −176.97.

### Step 2: Preparation of ethyl 2-[(15S,18S)-13,20,23-trifluoro-15-{5-[2-(3-fluoroazetidin-1-yl)ethyl]-2-oxo-4-(trifluoromethyl)pyridin-1-yl}-3,21-dimethyl-16-oxo-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetate

A mixture of ethyl (3S)-3-{2,4-difluoro-2′-hydroxy-5,6′-dimethyl-[1,1′-biphenyl]-3-yl}-3-{2-[2-fluoro-5-(hydroxymethyl)phenyl]-2-{5-[2-(3-fluoroazetidin-1-yl)ethyl]-2-oxo-4-(trifluoromethyl)pyridin-1-yl}acetamido}propanoate (240 mg, 0.30 mmol, 1.00 equiv.) and 2-(tributyl-lambda5-phosphanylidene)acetonitrile (297 mg, 1.23 mmol, 4.00 equiv.) in toluene (5 mL) was stirred overnight at 80° C. under nitrogen atmosphere. The mixture was allowed to cool down to room temperature. The resulting mixture was concentrated under reduced pressure. The residue was purified by reversed-phase flash chromatography (C18 gel; mobile phase, MeCN in water (0.1% NH₃·H₂O), 30% to 70% gradient in 25 min; detector, UV 220 nm) to afford ethyl 2-[(15S,18S)-13,20,23-trifluoro-15-{5-[2-(3-fluoroazetidin-1-yl)ethyl]-2-oxo-4-(trifluoromethyl)pyridin-1-yl}-3,21-dimethyl-16-oxo-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetate (93 mg, 39.6%, mixture) as a white solid. LC-MS: (ES+H, m/z): [M+H]⁺=760.0.

### Step 3: Preparation of [(15S,18S)-13,20,23-trifluoro-15-{5-[2-(3-fluoroazetidin-1-yl)ethyl]-2-oxo-4-(trifluoromethyl)pyridin-1-yl}-3,21-dimethyl-16-oxo-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetic acid & [(15R,18S)-13,20,23-trifluoro-15-{5-[2-(3-fluoroazetidin-1-yl)ethyl]-2-oxo-4-(trifluoromethyl)pyridin-1-yl}-3,21-dimethyl-16-oxo-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetic acid

To a stirred solution of ethyl 2-[(15S,18S)-13,20,23-trifluoro-15-{5-[2-(3-fluoroazetidin-1-yl)ethyl]-2-oxo-4-(trifluoromethyl)pyridin-1-yl}-3,21-dimethyl-16-oxo-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetate (80 mg, 0.10 mmol, 1.00 equiv., mixture) in THF (3 mL) was added LiOH·H₂O (5 mg, 0.21 mmol, 2.00 equiv.) in water (1 mL) dropwise at room temperature under nitrogen atmosphere. The resulting mixture was stirred for 5 h at room temperature under nitrogen atmosphere. The resulting mixture was concentrated under reduced pressure. The crude product was purified by Prep-Chiral-HPLC (Column: CHIRAL ART Cellulose-SB, 2*25 cm, 5 μm; Mobile Phase A: Hexane (0.1% HCOOH)-HPLC, Mobile Phase B: EtOH; Flow rate: 20 mL/min; Gradient: isocratic 20; Wavelength: 202/220 nm; RT1 (min): 5.67; RT2 (min): 10; Sample Solvent: EtOH; Injection Volume: 0.8 mL; Number of Runs: 5) to afford [(15S,18S)-13,20,23-trifluoro-15-{5-[2-(3-fluoroazetidin-1-yl)ethyl]-2-oxo-4-(trifluoromethyl)pyridin-1-yl}-3,21-dimethyl-16-oxo-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetic acid (10.1 mg, 12.4%) as a white solid & [(15R,18S)-13,20,23-trifluoro-15-{5-[2-(3-fluoroazetidin-1-yl)ethyl]-2-oxo-4-(trifluoromethyl)pyridin-1-yl}-3,21-dimethyl-16-oxo-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetic acid (12.3 mg, 15.2%).

Example 87: LC-MS: (ES+H, m/z): [M+H]⁺=732.20. ¹H NMR (400 MHz, DMSO-d₆) δ 9.18 (d, J=9.3 Hz, 1H), 7.53-7.46 (m, 1H), 7.43 (d, J=7.3 Hz, 1H), 7.34 (t, J=7.9 Hz, 1H), 7.28-7.20 (m, 1H), 7.18-7.07 (m, 2H), 7.01 (dd, J=21.5, 8.0 Hz, 2H), 6.83 (d, J=11.1 Hz, 2H), 5.99 (q, J=8.7 Hz, 1H), 5.04 (d, J=10.5 Hz, 1H), 4.99-4.75 (m, 2H), 3.21-3.11 (m, 2H), 2.94-2.72 (m, 4H), 2.45-2.27 (m, 4H), 2.22-2.10 (m, 6H). ¹⁹F NMR (377 MHz, DMSO-d₆) δ −62.528, −113.652, −116.891, −117.103, −171.946.

Example 88: LC-MS: (ES+H, m/z): [M+H]⁺=732.20. ¹H NMR (400 MHz, DMSO-d₆) δ 9.47 (d, J=5.4 Hz, 1H), 7.49-7.41 (m, 1H), 7.34-7.20 (m, 2H), 7.17 (d, J=8.4 Hz, 1H), 7.14-6.89 (m, 4H), 6.80 (s, 1H), 5.48-5.39 (m, 1H), 5.30 (d, J=13.8 Hz, 1H), 5.12 (d, J=13.5 Hz, 1H), 4.82 (dt, J=57.8, 5.3 Hz, 1H), 3.22-3.09 (m, 2H), 2.96-2.70 (m, 4H), 2.44-2.18 (m, 7H), 2.05 (s, 3H). ¹⁹F NMR (377 MHz, DMSO-d₆) δ −62.524, −116.430, −117.781, −118.256, −176.917.

### Example 89

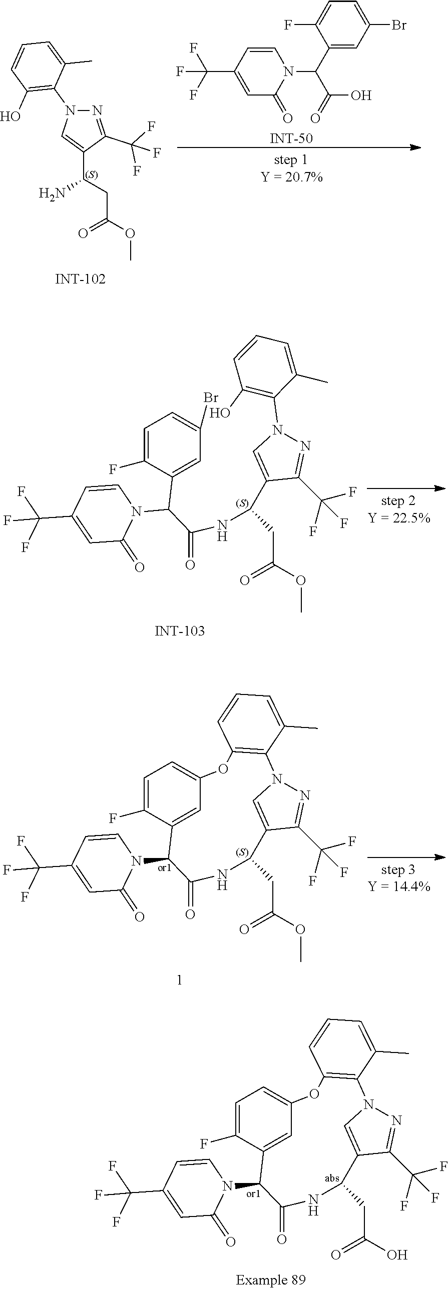

### Step 1: Preparation of methyl (3S)-3-[2-(5-bromo-2-fluorophenyl)-2-[2-oxo-4-(trifluoromethyl)pyridin-1-yl]acetamido]-3-[1-(2-hydroxy-6-methylphenyl)-3-(trifluoromethyl)pyrazol-4-yl]propanoate

A mixture of methyl (3S)-3-amino-3-[1-(2-hydroxy-6-methylphenyl)-3-(trifluoromethyl)pyrazol-4-yl]propanoate (180 mg, 0.52 mmol, 1.00 equiv), (5-bromo-2-fluorophenyl)[2-oxo-4-(trifluoromethyl)pyridin-1-yl]acetic acid (206 mg, 0.52 mmol, 1.00 equiv), HOBT (141 mg, 1.04 mmol, 2.00 equiv), EDCI (301 mg, 1.57 mmol, 3.00 equiv.) and DIEA (203 mg, 1.57 mmol, 3.00 equiv.) in MeCN (3 mL) was stirred for overnight at 40° C. under nitrogen atmosphere. The resulting mixture was concentrated under reduced pressure. The residue was purified by reversed-phase flash chromatography (C18 silica gel; mobile phase, MeCN in water (0.1% NH₃·H₂O), 40% to 70% gradient in 10 min; detector, UV 254 nm) to provide methyl (3S)-3-[2-(5-bromo-2-fluorophenyl)-2-[2-oxo-4-(trifluoromethyl)pyridin-1-yl]acetamido]-3-[1-(2-hydroxy-6-methylphenyl)-3-(trifluoromethyl)pyrazol-4-yl]propanoate (110 mg, 29.1%, mixture) as an off-white solid. LC-MS: (ES+H, m/z): [M+H]⁺=719.1.

### Step 2: Preparation methyl 2-[(13S,16S)-18-fluoro-7-methyl-15-oxo-16-[2-oxo-4-(trifluoromethyl)pyridin-1-yl]-11-(trifluoromethyl)-2-oxa-9,10,14-triazatetracyclo[15.3.1.1\hat{ }{9,12}.0\hat{ }{3,8}]docosa-1(20),3(8),4,6,10,12(22),17(21),18-octaen-13-yl]acetate

A mixture of methyl (3S)-3-[2-(5-bromo-2-fluorophenyl)-2-[2-oxo-4-(trifluoromethyl)pyridin-1-yl]acetamido]-3-[1-(2-hydroxy-6-methylphenyl)-3-(trifluoromethyl)pyrazol-4-yl]propanoate (100 mg, 0.13 mmol, 1.00 equiv), (1R,2R)-1-N,2-N-dimethylcyclohexane-1,2-diamine (19 mg, 0.13 mmol, 1.00 equiv.), CuI (13 mg, 0.07 mmol, 0.50 equiv.) and K₂CO₃ (9 mg, 0.07 mmol, 0.50 equiv.) in 1,4-dioxane (2 mL) was stirred for 2 h at 100° C. under nitrogen atmosphere. The mixture was allowed to cool down to room temperature. The resulting mixture was concentrated under reduced pressure. The residue was purified by reversed-phase flash chromatography (C18 gel; mobile phase, MeCN in water (0.10% NH₃·H₂O), 60% to 80% gradient in 10 min; detector, UV 254 nm) to provide methyl 2-[(13S,16S)-18-fluoro-7-methyl-15-oxo-16-[2-oxo-4-(trifluoromethyl)pyridin-1-yl]-11-(trifluoromethyl)-2-oxa-9,10,14-triazatetracyclo[15.3.1.1\hat{ }{9,12}.0\hat{ }{3,8}]docosa-1(20),3(8),4,6,10,12(22),17(21),18-octaen-13-yl]acetate (20 mg, 22.5%) as a white solid. LC-MS: (ES+H, m/z): [M+H]⁺=639.2.

### Step 3: Preparation of [(13S,16R*)-18-fluoro-7-methyl-15-oxo-16-[2-oxo-4-(trifluoromethyl)pyridin-1-yl]-11-(trifluoromethyl)-2-oxa-9,10,14-triazatetracyclo[15.3.1.1\hat{ }{9,12}.0\hat{ }{3,8}]docosa-1(20),3(8),4,6,10,12(22),17(21),18-octaen-13-yl]acetic acid

To a stirred mixture of methyl 2-[(13S,16S)-18-fluoro-7-methyl-15-oxo-16-[2-oxo-4-(trifluoromethyl)pyridin-1-yl]-11-(trifluoromethyl)-2-oxa-9,10,14-triazatetracyclo[15.3.1.1\hat{ }{9,12}.0\hat{ }{3,8}]docosa-1(20),3(8),4,6,10,12(22),17(21),18-octaen-13-yl]acetate (15 mg, 0.02 mmol, 1.00 equiv.) in THF (1 mL) and H₂O (0.2 mL) was added LiOH·H₂O (2.96 mg, 0.07 mmol, 3.00 equiv.) at room temperature. The resulting mixture was stirred for 2 h at room temperature under nitrogen atmosphere. The resulting mixture was concentrated under reduced pressure. The crude product (15 mg) was purified by Prep-HPLC (Column: Xselect CSH Fluoro-Phenyl Column, 30*150 mm, 5 μm; Mobile Phase A: Water (0.1% HCOOH), Mobile Phase B: MeCN; Flow rate: 60 mL/min; Gradient: 42% B to 58% B in 10 min; Wavelength: 254 nm/220 nm; RT1 (min): 6.55) to afford [(13S,16S)-18-fluoro-7-methyl-15-oxo-16-[2-oxo-4-(trifluoromethyl)pyridin-1-yl]-11-(trifluoromethyl)-2-oxa-9,10,14-triazatetracyclo[15.3.1.1\hat{ }{9,12}.0\hat{ }{3,8}]docosa-1(20),3(8),4,6,10,12(22),17(21),18-octaen-13-yl]acetic acid (2.2 mg, 14.4%) as a white solid. LC-MS: (ES+H, m/z): [M−H]⁻=623.00. ¹H NMR (300 MHz, DMSO-d₆) δ 9.10 (d, J=8.4 Hz, 1H), 7.65 (dt, J=15.7, 8.0 Hz, 2H), 7.49-7.24 (m, 4H), 7.07 (s, 1H), 6.88 (s, 1H), 6.68 (s, 1H), 6.39 (dd, J=7.3, 2.2 Hz, 1H), 5.80 (dd, J=6.2, 2.8 Hz, 1H), 5.34 (d, J=7.8 Hz, 1H), 2.70 (t, J=7.2 Hz, 2H), 2.00 (s, 3H). ¹⁹F NMR (282 MHz, DMSO-d₆) δ −58.37, −65.43, −120.76.

### Example 90

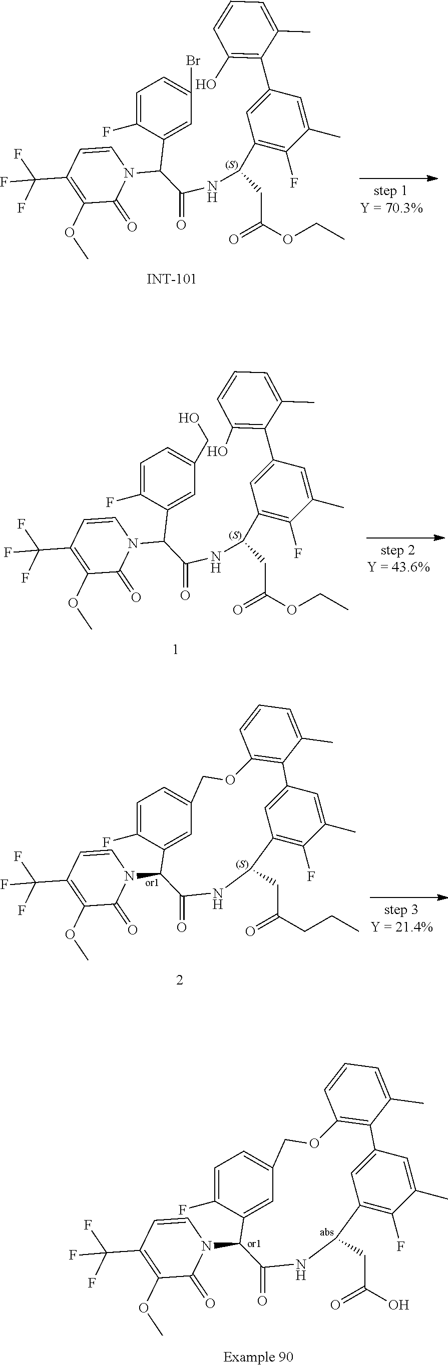

### Step 1: Preparation of ethyl (3S)-3-{4-fluoro-2′-hydroxy-5,6′-dimethyl-[1,1′-biphenyl]-3-yl}-3-{2-[2-fluoro-5-(hydroxymethyl)phenyl]-2-[3-methoxy-2-oxo-4-(trifluoromethyl)pyridin-1-yl]acetamido}propanoate

To a stirred solution of ethyl (3S)-3-[2-(5-bromo-2-fluorophenyl)-2-[3-methoxy-2-oxo-4-(trifluoromethyl)pyridin-1-yl]acetamido]-3-{4-fluoro-2′-hydroxy-5,6′-dimethyl-[1,1′-biphenyl]-3-yl}propanoate (370 mg, 0.50 mmol, 1.00 equiv.) and (tributylstannyl)methanol (483 mg, 1.50 mmol, 3.00 equiv.) in 1,4-dioxane (20 mL) was added dicyclohexyl[2′,4′,6′-tris(propan-2-yl)-[1,1′-biphenyl]-2-yl]phosphane; {2′-amino-[1,1′-biphenyl]-2-yl}(chloro)palladium (39 mg, 0.05 mmol, 0.10 equiv.) in portions at room temperature under nitrogen atmosphere. The resulting mixture was stirred overnight at 90° C. under nitrogen atmosphere. The mixture was allowed to cool down to room temperature. The resulting mixture was filtered, and the filter cake was washed with EtOAc (100 mL). The filtrate was concentrated under reduced pressure. The residue was purified by reversed-phase flash chromatography (C18 gel; mobile phase, CH₃CN in water (10 mmol/L NH₄HCO₃), 40% to 70% gradient in 15 min; detector, UV 254 nm) to afford ethyl (3S)-3-{4-fluoro-2′-hydroxy-5,6′-dimethyl-[1,1′-biphenyl]-3-yl}-3-{2-[2-fluoro-5-(hydroxymethyl)phenyl]-2-[3-methoxy-2-oxo-4-(trifluoromethyl)pyridin-1-yl]acetamido}propanoate (243 mg, 70.3%, mixture) as an off-white solid. LC-MS: (ES+H, m/z): [M+H]⁺=689.25. ¹H NMR (400 MHz, DMSO-d₆) δ 9.32 (dd, J=19.5, 8.2 Hz, 1H), 7.49-7.35 (m, 1H), 7.27-7.16 (m, 1H), 7.08-6.97 (m, 4H), 6.86 (d, J=7.0 Hz, 1H), 6.75-6.67 (m, 2H), 6.32 (dd, J=18.7, 7.5 Hz, 1H), 5.64-5.54 (m, 1H), 4.54 (s, 1H), 4.29 (d, J=4.0 Hz, 1H), 4.04-3.95 (m, 2H), 3.92 (s, 1H), 3.85 (s, 1H), 2.90-2.65 (m, 2H), 2.24 (d, J=1.9 Hz, 3H), 1.89 (d, J=25.4 Hz, 3H), 1.10-10.1 (m, 3H). ¹⁹F NMR (400 MHz, DMSO-d₆) δ −61.93, −61.97, −118.79, −119.00, −126.03, −126.86.

### Step 2: Preparation of ethyl 2-[(15S,18S)-13,20-difluoro-15-[3-methoxy-2-oxo-4-(trifluoromethyl)pyridin-1-yl]-3,21-dimethyl-16-oxo-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetate

To mixture of ethyl (3S)-3-{4-fluoro-2′-hydroxy-5,6′-dimethyl-[1,1′-biphenyl]-3-yl}-3-{2-[2-fluoro-5-(hydroxymethyl)phenyl]-2-[3-methoxy-2-oxo-4-(trifluoromethyl)pyridin-1-yl]acetamido}propanoate (200 mg, 0.29 mmol, 1.00 equiv.) and CMBP (350 mg, 1.45 mmol, 5.00 equiv.) in toluene (10 mL) was stirred overnight at 100° C. under a nitrogen atmosphere. The mixture was allowed to cool down to room temperature. The resulting mixture was diluted with EtOAc (100 mL). The organic layers were washed with water (2×20 mL), dried over anhydrous Na₂SO₄, filtered, and concentrated. The residue was purified by reversed-phase flash chromatography (C18 gel; mobile phase, CH₃CN in water (0.1% NH₃·H₂O), 0% to 70% gradient in 40 min; detector, UV 220 nm) to provide ethyl 2-[(15S,18S)-13,20-difluoro-15-[3-methoxy-2-oxo-4-(trifluoromethyl)pyridin-1-yl]-3,21-dimethyl-16-oxo-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetate (85 mg, 43.6%) as a brown oil. LC-MS: (ES+H, m/z): [M+H]⁺=671.20. ¹H NMR (400 MHz, DMSO-d₆) δ 9.48 (d, J=7.5 Hz, 1H), 7.58-7.51 (m, 1H), 7.29 (d, J=8.3 Hz, 1H), 7.25-7.17 (m, 2H), 7.13-7.08 (m, 1H), 7.03-7.00 (m, 1H), 6.95 (s, 1H), 6.83 (d, J=7.5 Hz, 1H), 6.53-6.48 (m, 1H), 6.25 (d, J=7.5 Hz, 1H), 5.59 (t, J=11.2 Hz, 1H), 5.37 (d, J=13.2 Hz, 1H), 4.97 (d, J=13.2 Hz, 1H), 4.16-3.96 (m, 3H), 3.94 (s, 3H), 2.76 (dd, J=15.8, 5.1 Hz, 1H), 2.64 (dd, J=15.7, 9.8 Hz, 1H), 2.28 (d, J=1.8 Hz, 3H), 1.91 (s, 3H), 1.13 (t, J=7.1 Hz, 3H). ¹⁹F NMR (400 MHz, DMSO-d₆) δ −61.96, −117.22, −127.16.

### Step 3: Preparation of [(15S,18S)-13,20-difluoro-15-[3-methoxy-2-oxo-4-(trifluoromethyl)pyridin-1-yl]-3,21-dimethyl-16-oxo-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetic acid

To a stirred mixture of ethyl 2-[(15S,18S)-13,20-difluoro-15-[3-methoxy-2-oxo-4-(trifluoromethyl)pyridin-1-yl]-3,21-dimethyl-16-oxo-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetate (110 mg, 0.16 mmol, 1.00 equiv.) in THF (5 mL) was added LiOH·H₂O (13 mg, 0.32 mmol, 2.00 equiv.) in water (2.5 mL) at room temperature under nitrogen atmosphere. The resulting mixture was stirred for 1 h at room temperature under nitrogen atmosphere. The resulting mixture was diluted with water (2.5 mL). The mixture was acidified to pH 5 with 1 M HCl (aq.). The resulting mixture was concentrated under vacuum. The crude product (100 mg) was purified by Prep-HPLC (Column: XBridge Prep OBD C18 Column, 30*150 mm, 10 μm; Mobile Phase A: Water (10 mmol/L NH₄HCO₃+0.05% NH₃H₂O), Mobile Phase B: CH₃CN; Flow rate: 60 mL/min; Gradient: 27% B to 44% B in 8 min; Wavelength: 254 nm/220 nm; RT1 (min): 9.65) to afford [(15S,18S)-13,20-difluoro-15-[3-methoxy-2-oxo-4-(trifluoromethyl)pyridin-1-yl]-3,21-dimethyl-16-oxo-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetic acid (22.6 mg, 21.4%) as a white solid. LC-MS: (ES+H, m/z): [M+H]⁺=643.15. ¹H NMR (400 MHz, CD₃OD) δ 7.47-7.43 (m, 1H), 7.28 (dd, J=7.4, 2.1 Hz, 1H), 7.20-7.05 (m, 4H), 6.94 (d, J=7.4 Hz, 2H), 6.82 (d, J=7.1 Hz, 1H), 6.62 (dd, J=6.8, 2.2 Hz, 1H), 6.26 (d, J=7.5 Hz, 1H), 5.76 (dd, J=8.7, 5.6 Hz, 1H), 5.29 (d, J=13.2 Hz, 1H), 4.95 (d, J=13.2 Hz, 1H), 3.97 (s, 3H), 2.81-2.67 (m, 2H), 2.31 (d, J=2.0 Hz, 3H), 1.96 (s, 3H). ¹⁹F NMR (400 MHz, CD₃OD) δ −64.60, −118.50, −128.30.

### Example 91

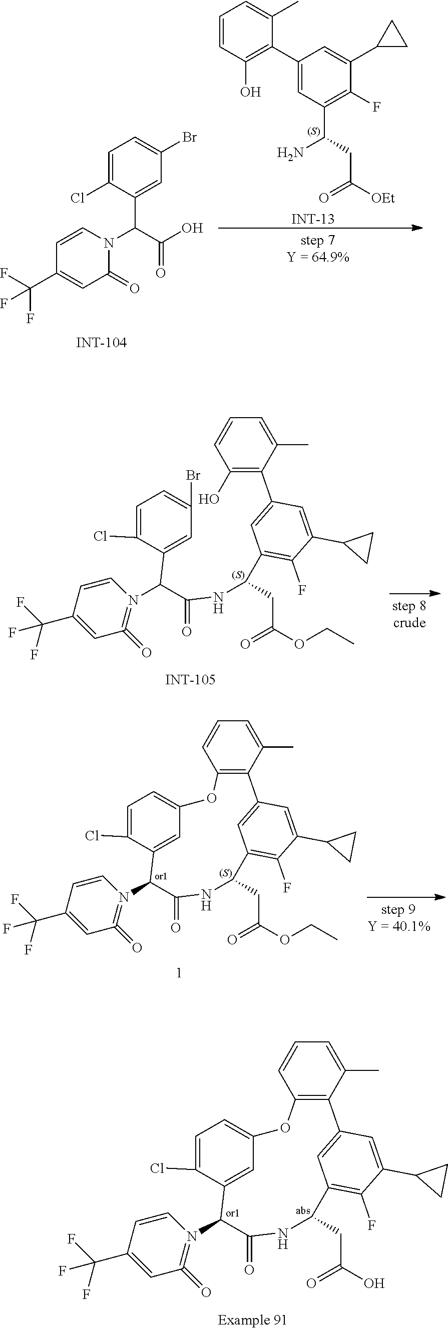

### Step 1: Preparation of ethyl (3S)-3-[2-(5-bromo-2-chlorophenyl)-2-[2-oxo-4-(trifluoromethyl)pyridin-1-yl]acetamido]-3-{5-cyclopropyl-4-fluoro-2′-hydroxy-6′-methyl-[1,1′-biphenyl]-3-yl}propanoate

To a stirred solution of (5-bromo-2-chlorophenyl)[2-oxo-4-(trifluoromethyl)pyridin-1-yl]acetic acid (379 mg, 0.92 mmol, 1.00 equiv.) and HOBT (162 mg, 1.20 mmol, 1.30 equiv.) in CH₃CN (5 mL) were added EDCI (265 mg, 1.38 mmol, 1.50 equiv.) and DIEA (477 mg, 3.69 mmol, 4.00 equiv.) in portions at room temperature under nitrogen atmosphere. The resulting mixture was stirred for 1 h at 40° C. under nitrogen atmosphere. Then to the above mixture was added ethyl (3S)-3-amino-3-{5-cyclopropyl-4-fluoro-2′-hydroxy-6′-methyl-[1,1′-biphenyl]-3-yl}propanoate (330 mg, 0.92 mmol, 1.00 equiv.) dropwise at room temperature. The resulting mixture was stirred overnight at 40° C. The mixture was allowed to cool down to room temperature. The resulting mixture was concentrated under reduced pressure. The residue was purified by reversed-phase flash chromatography (C18 gel; mobile phase, CH₃CN in water (10 mmol/L NH₄HCO₃), 40% to 70% gradient in 20 min; detector, UV 254 nm) to afford ethyl (3S)-3-[2-(5-bromo-2-chlorophenyl)-2-[2-oxo-4-(trifluoromethyl)pyridin-1-yl]acetamido]-3-{5-cyclopropyl-4-fluoro-2′-hydroxy-6′-methyl-[1,1′-biphenyl]-3-yl}propanoate (450 mg, 64.9%, mixture) as an off-white solid. LC-MS: (ES+H, m/z): [M+H]⁺=749.10. ¹H NMR (400 MHz, DMSO-d₆) δ 9.47 (dd, J=13.2, 8.2 Hz, 1H), 9.07 (d, J=4.6 Hz, 1H), 7.75-7.64 (m, 1H), 7.57-7.49 (m, 1H), 7.29-7.21 (m, 1H), 7.08-6.83 (m, 3H), 6.79-6.65 (m, 4H), 6.41-6.34 (m, 1H), 5.71-5.57 (m, 1H), 4.13-3.96 (m, 2H), 2.94-2.78 (m, 1H), 2.15-2.02 (m, 1H), 1.89 (d, J=18.7 Hz, 3H), 1.09 (q, J=7.3 Hz, 3H), 1.02-0.92 (m, 2H), 0.72-0.67 (m, 2H). ¹⁹F NMR (400 MHz, DMSO-d₆) δ −65.39, −65.44, −129.66, −130.28.

### Step 2: Preparation of ethyl 2-[(14S,17S)-12-chloro-20-cyclopropyl-19-fluoro-3-methyl-15-oxo-14-[2-oxo-4-(trifluoromethyl)pyridin-1-yl]-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate

To a stirred solution of ethyl (3S)-3-[2-(5-bromo-2-chlorophenyl)-2-[2-oxo-4-(trifluoromethyl)pyridin-1-yl]acetamido]-3-{5-cyclopropyl-4-fluoro-2′-hydroxy-6′-methyl-[1,1′-biphenyl]-3-yl}propanoate (200 mg, 0.27 mmol, 1.00 equiv.) and (1S,2S)—N1,N2-dimethylcyclohexane-1,2-diamine (37 mg, 0.26 mmol, 1.00 equiv.) in 1,4-dioxane (20 mL) were added K₂CO₃ (73 mg, 0.53 mmol, 2.00 equiv.) and CuI (25 mg, 0.13 mmol, 0.50 equiv.) in portions at room temperature under nitrogen atmosphere. The resulting mixture was stirred overnight at 100° C. under nitrogen atmosphere. The mixture was allowed to cool down to room temperature. The resulting mixture was filtered, and the filter cake was washed with CH₂Cl₂ (100 mL). The filtrate was concentrated under reduced pressure. The residue was purified by reversed-phase flash chromatography (C18 gel; mobile phase, CH₃CN in water (10 mmol/L NH₄HCO₃), 40% to 70% gradient in 15 min; detector, UV 254 nm) to afford ethyl 2-[(14S,17S)-12-chloro-20-cyclopropyl-19-fluoro-3-methyl-15-oxo-14-[2-oxo-4-(trifluoromethyl)pyridin-1-yl]-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (110 mg, crude) as an off-white solid. LC-MS: (ES+H, m/z): [M+H]⁺=669.20. ¹H NMR (400 MHz, DMSO-d₆) δ 9.26 (d, J=7.3 Hz, 1H), 7.66-7.60 (m, 2H), 7.53 (d, J=8.8 Hz, 1H), 7.37 (t, J=7.9 Hz, 1H), 7.22 (dd, J=20.4, 7.4 Hz, 2H), 6.88 (s, 1H), 6.70-6.63 (m, 2H), 6.43 (d, J=6.0 Hz, 1H), 6.32 (dd, J=7.4, 2.1 Hz, 1H), 6.06 (d, J=2.7 Hz, 1H), 5.52 (q, J=7.6 Hz, 1H), 4.16-4.00 (m, 2H), 2.86-2.73 (m, 2H), 2.17-2.13 (m, 1H), 1.94 (s, 3H), 1.13 (t, J=7.1 Hz, 3H), 1.01 (dd, J 8.4, 2.8 Hz, 2H), 0.80 (d, J=5.3 Hz, 1H), 0.72 (d, J=5.1 Hz, 1H).

### Step 3: Preparation of [(14S,17S)-12-chloro-20-cyclopropyl-19-fluoro-3-methyl-15-oxo-14-[2-oxo-4-(trifluoromethyl)pyridin-1-yl]-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid

To a stirred mixture of ethyl 2-[(14S,17S)-12-chloro-20-cyclopropyl-19-fluoro-3-methyl-15-oxo-14-[2-oxo-4-(trifluoromethyl)pyridin-1-yl]-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (100 mg, 0.14 mmol, 1.00 equiv.) in THF (8 mL) was added LiOH·H₂O (12 mg, 0.29 mmol, 2.00 equiv.) in water (4 mL) at room temperature under nitrogen atmosphere. The resulting mixture was stirred for 1 h at room temperature under nitrogen atmosphere. The resulting mixture was diluted with water (4 mL). The mixture was acidified to pH 5 with 1 M HCl (aq.). The resulting mixture was concentrated under vacuum. The crude product (130 mg) was purified by Prep-HPLC (Column: XBridge Shield RP18 OBD Column, 30*150 mm, 5 μm; Mobile Phase A: water (10 mmol/L NH₄HCO₃), Mobile Phase B: CH₃CN; Flow rate: 60 mL/min; Gradient: 32% B to 42% B in 10 min; Wavelength: 254 nm/220 nm; RT1 (min): 9.1) to afford [(14S,17S)-12-chloro-20-cyclopropyl-19-fluoro-3-methyl-15-oxo-14-[2-oxo-4-(trifluoromethyl)pyridin-1-yl]-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid (38.5 mg, 40.1%) as a white solid. LC-MS: (ES+H, m/z): [M+H]⁺=641.10. ¹H NMR (400 MHz, CD₃OD) δ 7.55 (dd, J=8.9, 2.8 Hz, 1H), 7.48-7.42 (m, 2H), 7.33 (t, J=7.9 Hz, 1H), 7.24 (d, J=7.3 Hz, 1H), 7.15 (d, J=7.6 Hz, 1H), 6.82 (d, J=21.4 Hz, 2H), 6.63 (dd, J=6.8, 2.2 Hz, 1H), 6.47-6.39 (m, 2H), 6.23 (d, J=2.8 Hz, 1H), 5.74 (t, J=7.4 Hz, 1H), 2.79-2.62 (m, 2H), 2.17-2.15 (m, 1H), 1.98 (s, 3H), 1.00 (dd, J=8.4, 2.7 Hz, 2H), 0.80-0.64 (m, 2H). ¹⁹F NMR (400 MHz, CD₃OD) δ −68.17, −129.90.

### Example 92

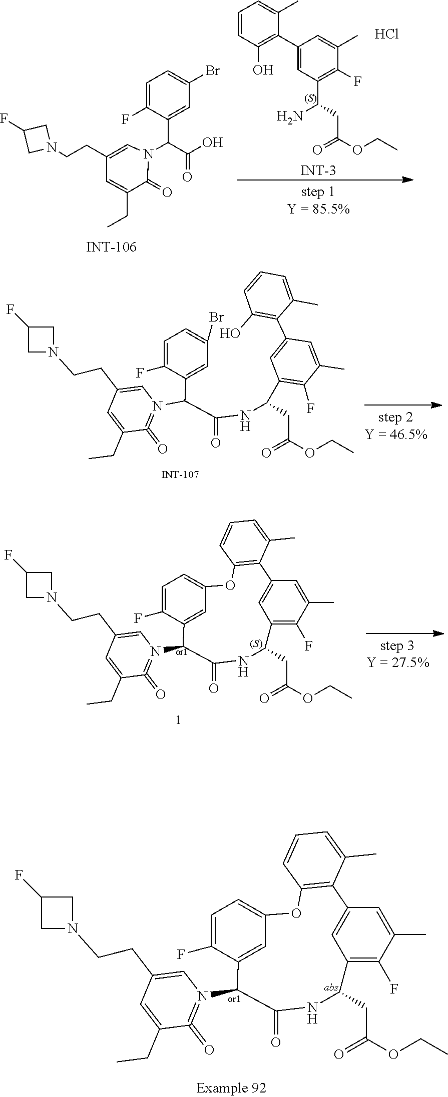

### Step 1: Preparation of ethyl (3S)-3-(2-(5-bromo-2-fluorophenyl)-2-(3-ethyl-5-(2-(3-fluoroazetidin-1-yl)ethyl)-2-oxopyridin-1(2H)-yl)acetamido)-3-(4-fluoro-2′-hydroxy-5,6′-dimethyl-[1,1′-biphenyl]-3-yl)propanoate

A mixture of ethyl (S)-3-amino-3-(4-fluoro-2′-hydroxy-5,6′-dimethyl-[1,1′-biphenyl]-3-yl)propanoate hydrochloride (95 mg, 0.26 mmol, 1.30 equiv.) and NEt₃ (120 mg, 1.19 mmol, 6.00 equiv.) in MeCN (2 mL) was stirred for 30 min at 30° C. under nitrogen atmosphere. The resulting mixture was concentrated under reduced pressure. This resulted in ethyl (S)-3-amino-3-(4-fluoro-2′-hydroxy-5,6′-dimethyl-[1,1′-biphenyl]-3-yl)propanoate. To a stirred solution of 2-(5-bromo-2-fluorophenyl)-2-(3-ethyl-5-(2-(3-fluoroazetidin-1-yl)ethyl)-2-oxopyridin-1(2H)-yl)acetic acid (90 mg, 0.20 mmol, 1.00 equiv.) in MeCN (2 mL) were added HOBT (35 mg, 0.26 mmol, 1.30 equiv.) and EDCI (57 mg, 0.30 mmol, 1.50 equiv.). The resulting mixture was stirred for 1 h at 30° C. under nitrogen atmosphere. To the above mixture was added ethyl (S)-3-amino-3-(4-fluoro-2′-hydroxy-5,6′-dimethyl-[1,1′-biphenyl]-3-yl)propanoate in MeCN (1 mL) dropwise at room temperature. The resulting mixture was stirred for additional overnight at 30° C. The mixture was allowed to cool down to room temperature. The resulting mixture was concentrated under reduced pressure. The residue was purified by reversed-phase flash chromatography (C18 gel; mobile phase, MeCN in water (0.1% NH₃·H₂O), 40% to 70% gradient in 25 min; detector, UV 220 nm) to provide ethyl (3S)-3-(2-(5-bromo-2-fluorophenyl)-2-(3-ethyl-5-(2-(3-fluoroazetidin-1-yl)ethyl)-2-oxopyridin-1(2H)-yl)acetamido)-3-(4-fluoro-2′-hydroxy-5,6′-dimethyl-[1,1′-biphenyl]-3-yl)propanoate (130 mg, mixture, 85.5%) as a white solid. LC-MS: (ES+H, m/z): [M+H]⁺=768.2/770.2. ¹1H NMR (400 MHz, CD₃OD) δ 7.65-7.53 (m, 1H), 7.26 (d, J=23.4 Hz, 2H), 7.16-6.93 (m, 5H), 6.91 (d, J=2.2 Hz, 1H), 6.70 (dd, J=17.1, 7.9 Hz, 2H), 5.71 (d, J=7.1 Hz, 1H), 5.14-5.06 (m, 1H), 4.16-4.02 (m, 2H), 3.57-3.45 (m, 2H), 3.22-3.03 (m, 2H), 3.03-2.82 (m, 2H), 2.69-2.44 (m, 4H), 2.41-2.24 (m, 5H), 1.95 (d, J=22.0 Hz, 3H), 1.21-1.10 (m, 6H).

### Step 2: Preparation of ethyl 2-[(14R*,17S)-14-{3-ethyl-5-[2-(3-fluoroazetidin-1-yl)ethyl]-2-oxopyridin-1-yl}-12,19-difluoro-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (Intermediate 107)

To a stirred mixture of ethyl (3S)-3-(2-(5-bromo-2-fluorophenyl)-2-(3-ethyl-5-(2-(3-fluoroazetidin-1-yl)ethyl)-2-oxopyridin-1(2H)-yl)acetamido)-3-(4-fluoro-2′-hydroxy-5,6′-dimethyl-[1,1′-biphenyl]-3-yl)propanoate (120 mg, 0.16 mmol, 1.00 equiv.) and (1R,2R)—N1,N2-dimethylcyclohexane-1,2-diamine (22 mg, 0.16 mmol, 1.00 equiv.) in dioxane (4 mL) were added CuI (15 mg, 0.08 mmol, 0.50 equiv.) and K₂CO₃ (43 mg, 0.31 mmol, 2.00 equiv.). The resulting mixture was stirred overnight at 100° C. under nitrogen atmosphere. The mixture was allowed to cool down to room temperature. The resulting mixture was diluted with water (30 mL). The resulting mixture was extracted with EtOAc (3×30 mL). The combined organic layers were washed with brine (3×30 mL), dried over anhydrous Na₂SO₄, filtered, and concentrated. The residue was purified by reversed-phase flash chromatography (C18 gel; mobile phase, MeCN in water (0.1% NH₃·H₂O), 40% to 70% gradient in 20 min; detector, UV 220 nm) to provide ethyl 2-[(14R*,17S)-14-{3-ethyl-5-[2-(3-fluoroazetidin-1-yl)ethyl]-2-oxopyridin-1-yl}-12,19-difluoro-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (50 mg, 46.5%) as a yellow solid. LC-MS: (ES+H, m/z): [M+H]⁺=688.3. ¹H NMR (400 MHz, CD₃OD) δ 7.51 (dt, J=9.1, 3.6 Hz, 1H), 7.44 (d, J=8.0 Hz, 1H), 7.38-7.25 (m, 2H), 7.20-7.07 (m, 2H), 7.01-6.94 (m, 1H), 6.87-6.79 (m, 2H), 6.44 (dd, J=6.4, 2.3 Hz, 1H), 6.05 (dd, J=6.2, 3.0 Hz, 1H), 5.69 (t, J=7.9 Hz, 1H), 5.08-4.92 (m, 1H), 4.10 (qd, J=7.1, 2.7 Hz, 2H), 3.46 (dq, J=23.9, 7.9, 6.8 Hz, 2H), 3.11 (tdd, J=22.3, 9.1, 4.4 Hz, 2H), 2.91-2.80 (m, 1H), 2.71 (dd, J=15.4, 7.8 Hz, 1H), 2.56 (dt, J=20.8, 7.4 Hz, 4H), 2.39-2.24 (m, 5H), 2.01 (d, J=14.6 Hz, 3H), 1.18 (t, J=7.3 Hz, 6H).

### Step 3: Preparation of [(14R*,17S)-14-{3-ethyl-5-[2-(3-fluoroazetidin-1-yl)ethyl]-2-oxopyridin-1-yl}-12,19-difluoro-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid

A mixture of ethyl 2-[(14R*,17S)-14-{3-ethyl-5-[2-(3-fluoroazetidin-1-yl)ethyl]-2-oxopyridin-1-yl}-12,19-difluoro-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (40 mg, 0.06 mmol, 1.00 equiv.) and LiOH·H₂O (10 mg, 0.23 mmol, 4.00 equiv.) in THF (2 mL) and H₂O (0.5 mL) was stirred overnight at room temperature under nitrogen atmosphere. The mixture was acidified to pH 5 with HCl (aq. 3 N). The resulting mixture was concentrated under reduced pressure. The crude product was purified by Prep-HPLC (Column: XBridge Shield RP18 OBD Column, 30*150 mm, 5 μm; Mobile Phase A: Water (10 mmol/L NH₄HCO₃), Mobile Phase B: MeCN; Flow rate: 60 mL/min; Gradient: 20% B to 40% B in 10 min; Wavelength: 254 nm/220 nm; RT1 (min): 9.1) to afford [(14R*,17S)-14-{3-ethyl-5-[2-(3-fluoroazetidin-1-yl)ethyl]-2-oxopyridin-1-yl}-12,19-difluoro-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid (10.8 mg, 27.5%) as a white solid. LC-MS: (ES+H, m/z): [M+H]⁺=660.30. ¹H NMR (400 MHz, CD₃OD) δ 7.49 (dt, J=9.0, 3.6 Hz, 1H), 7.41 (d, J=8.0 Hz, 1H), 7.36-7.25 (m, 2H), 7.18-7.05 (m, 2H), 6.96-6.80 (m, 3H), 6.49 (dd, J=6.4, 2.3 Hz, 1H), 6.09 (dd, J=6.2, 3.0 Hz, 1H), 5.69 (t, J=7.4 Hz, 1H), 5.17-4.98 (m, 1H), 3.75 (qd, J=17.6, 7.6 Hz, 2H), 3.53-3.34 (m, 2H), 2.81 (t, J=7.3 Hz, 2H), 2.66 (qd, J=15.2, 7.4 Hz, 2H), 2.53 (q, J=7.5 Hz, 2H), 2.43-2.27 (m, 5H), 1.98 (s, 3H), 1.18 (t, J=7.5 Hz, 3H). ¹⁹F NMR (376 MHz, CD₃OD) δ −123.304, −126.745, −180.729.

### Example 93

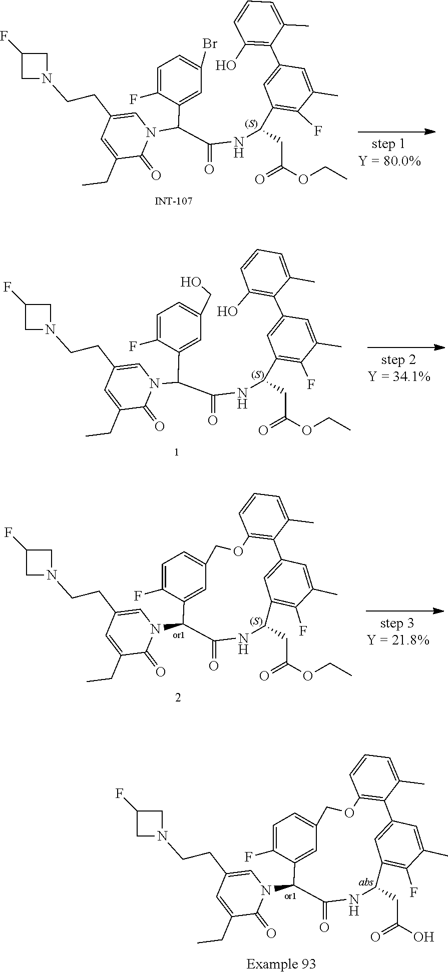

### Step 1: Preparation of ethyl (3S)-3-(2-(3-ethyl-5-(2-(3-fluoroazetidin-1-yl)ethyl)-2-oxopyridin-1(2H)-yl)-2-(2-fluoro-5-(hydroxymethyl)phenyl)acetamido)-3-(4-fluoro-2′-hydroxy-5,6′-dimethyl-[1,1′-biphenyl]-3-yl)propanoate

To a stirred mixture of ethyl (3S)-3-(2-(5-bromo-2-fluorophenyl)-2-(3-ethyl-5-(2-(3-fluoroazetidin-1-yl)ethyl)-2-oxopyridin-1(2H)-yl)acetamido)-3-(4-fluoro-2′-hydroxy-5,6′-dimethyl-[1,1′-biphenyl]-3-yl)propanoate (200 mg, 0.26 mmol, 1.00 equiv.) and 2nd generation XPhos precatalyst (20 mg, 0.03 mmol, 0.10 equiv.) in dioxane (10 mL) was added (tributylstannyl)methanol (251 mg, 0.78 mmol, 3.00 equiv.). The resulting mixture was stirred overnight at 90° C. under nitrogen atmosphere. The mixture was allowed to cool down to room temperature. The resulting mixture was diluted with water (50 mL). The resulting mixture was extracted with EtOAc (3×50 mL). The combined organic layers were washed with brine (3×50 mL), dried over anhydrous Na₂SO₄, filtered, and concentrated. The residue was purified by reversed-phase flash chromatography (C18 gel; mobile phase, MeCN in water (0.1% NH₃·H₂O), 40% to 70% gradient in 20 min; detector, UV 220 nm) to provide ethyl (3S)-3-(2-(3-ethyl-5-(2-(3-fluoroazetidin-1-yl)ethyl)-2-oxopyridin-1(2H)-yl)-2-(2-fluoro-5-(hydroxymethyl)phenyl)acetamido)-3-(4-fluoro-2′-hydroxy-5,6′-dimethyl-[1,1′-biphenyl]-3-yl)propanoate (150 mg, 80.0%, mixture) as a yellow solid. LC-MS: (ES+H, m/z): [M+H]⁺=720.3. ¹H NMR (400 MHz, CD₃OD) δ 7.47 (dd, J=7.4, 4.9 Hz, 1H), 7.41 (ddd, J=8.0, 5.0, 2.2 Hz, 1H), 7.30-7.22 (m, 1H), 7.17-6.94 (m, 5H), 6.89 (dd, J=11.1, 2.4 Hz, 1H), 6.71 (ddd, J=17.2, 7.7, 2.8 Hz, 2H), 5.72 (q, J=7.7 Hz, 1H), 5.14-4.95 (m, 1H), 4.62 (d, J=25.4 Hz, 1H), 4.40 (s, 1H), 4.14-3.94 (m, 2H), 3.64-3.44 (m, 2H), 3.27-3.07 (m, 2H), 3.01-2.79 (m, 2H), 2.70-2.59 (m, 2H), 2.57-2.41 (m, 2H), 2.38-2.26 (m, 5H), 1.94 (d, J=12.5 Hz, 3H), 1.22-1.08 (m, 6H).

### Step 2: Preparation of ethyl 2-[(15S,18S)-15-{3-ethyl-5-[2-(3-fluoroazetidin-1-yl)ethyl]-2-oxopyridin-1-yl}-13,20-difluoro-3,21-dimethyl-16-oxo-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetate

To a stirred solution of ethyl (3S)-3-(2-(3-ethyl-5-(2-(3-fluoroazetidin-1-yl)ethyl)-2-oxopyridin-1(2H)-yl)-2-(2-fluoro-5-(hydroxymethyl)phenyl)acetamido)-3-(4-fluoro-2′-hydroxy-5,6′-dimethyl-[1,1′-biphenyl]-3-yl)propanoate (150 mg, 0.21 mmol, 1.00 equiv.) in toluene (10 mL) was added 2-(tributyl-lambda5-phosphanylidene)acetonitrile (251 mg, 1.04 mmol, 5.00 equiv.). The resulting mixture was stirred overnight at 100° C. under nitrogen atmosphere. The mixture was allowed to cool down to room temperature. The resulting mixture was concentrated under reduced pressure. The residue was purified by reversed-phase flash chromatography (C18 gel; mobile phase, MeCN in water (0.1% NH₃·H₂O), 40% to 70% gradient in 20 min; detector, UV 220 nm) to provide ethyl 2-[(15S,18 S)-15-{3-ethyl-5-[2-(3-fluoroazetidin-1-yl)ethyl]-2-oxopyridin-1-yl}-13,20-difluoro-3,21-dimethyl-16-oxo-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetate (50 mg, 34.1%) as a yellow solid. LC-MS: (ES+H, m/z): [M+H]⁺=702.4. ¹H NMR (400 MHz, CD₃OD) δ 7.44 (ddd, J=7.9, 4.9, 2.1 Hz, 1H), 7.30-7.24 (m, 2H), 7.20-7.12 (m, 2H), 7.10-7.04 (m, 2H), 6.98-6.92 (m, 1H), 6.82 (d, J=6.9 Hz, 1H), 6.74 (d, J=2.4 Hz, 1H), 6.62 (dd, J=6.8, 2.1 Hz, 1H), 5.76 (dd, J=8.7, 6.2 Hz, 1H), 5.30 (d, J=13.2 Hz, 1H), 5.10-4.90 (m, 2H), 4.22-4.03 (m, 2H), 3.55-3.40 (m, 2H), 3.21-3.03 (m, 2H), 2.80-2.70 (m, 2H), 2.64-2.51 (m, 4H), 2.30 (dd, J=13.2, 3.3 Hz, 5H), 1.97 (s, 3H), 1.19 (td, J=7.3, 2.2 Hz, 6H).

### Step 3: Preparation of [(15R*,18S)-15-{3-ethyl-5-[2-(3-fluoroazetidin-1-yl)ethyl]-2-oxopyridin-1-yl}-13,20-difluoro-3,21-dimethyl-16-oxo-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetic acid

A mixture of ethyl 2-[(15S,18S)-15-{3-ethyl-5-[2-(3-fluoroazetidin-1-yl)ethyl]-2-oxopyridin-1-yl}-13,20-difluoro-3,21-dimethyl-16-oxo-8-oxa-17-azatetracyclo [17.3.1.1\hat{ }{10,14}.0\hat{ }{2,7}]tetracosa-1(23),2(7),3,5,10,12,14(24),19,21-nonaen-18-yl]acetate (80 mg, 0.11 mmol, 1.00 equiv.) and LiOH·H₂O (19 mg, 0.46 mmol, 4.00 equiv.) in THF (2 mL) and H₂O (0.4 mL) was stirred for 4 h at room temperature under nitrogen atmosphere. The mixture was acidified to pH 5 with HCl (aq. 3 N). The resulting mixture was concentrated under reduced pressure. The crude product was purified by Prep-HPLC (Column: XBridge Prep OBD C18 Column, 30*150 mm, 10 μm; Mobile Phase A: water (10 mmol/L NH₄HCO₃+0.05% NH₃H₂O), Mobile Phase B: MeCN; Flow rate: 60 mL/min; Gradient: 22% B to 42% B in 8 min; Wavelength: 254 nm/220 nm; RT1 (min): 9.48) to afford [(15R*,18S)-15-{3-ethyl-5-[2-(3-fluoroazetidin-1-yl)ethyl]-2-oxopyridin-1-yl}-13,20-difluoro-3,21-dimethyl-16-oxo-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetic acid (17.2 mg, 21.8%) as a white solid. LC-MS: (ES+H, m/z): [M+H]⁺=674.25. ¹H NMR (400 MHz, CD₃OD) δ 7.45-7.38 (m, 1H), 7.33-7.30 (m, 1H), 7.25 (d, J=2.3 Hz, 1H), 7.19-7.02 (m, 4H), 6.93-6.88 (m, 1H), 6.83-6.76 (m, 2H), 6.73-6.68 (m, 1H), 5.71 (dd, J=8.0, 5.8 Hz, 1H), 5.27 (d, J=13.2 Hz, 1H), 5.19-5.01 (m, 1H), 4.97 (d, J=13.2 Hz, 1H), 3.80 (td, J=19.2, 18.3, 10.5 Hz, 2H), 3.58-3.36 (m, 2H), 2.82 (t, J=7.3 Hz, 2H), 2.66 (qd, J=15.4, 6.9 Hz, 2H), 2.53 (qd, J=7.5, 3.6 Hz, 2H), 2.41-2.25 (m, 5H), 1.96 (s, 3H), 1.17 (t, J=7.5 Hz, 3H). ¹⁹F NMR (377 MHz, CD₃OD) δ −118.212, −128.073, −180.475.

### Example 94

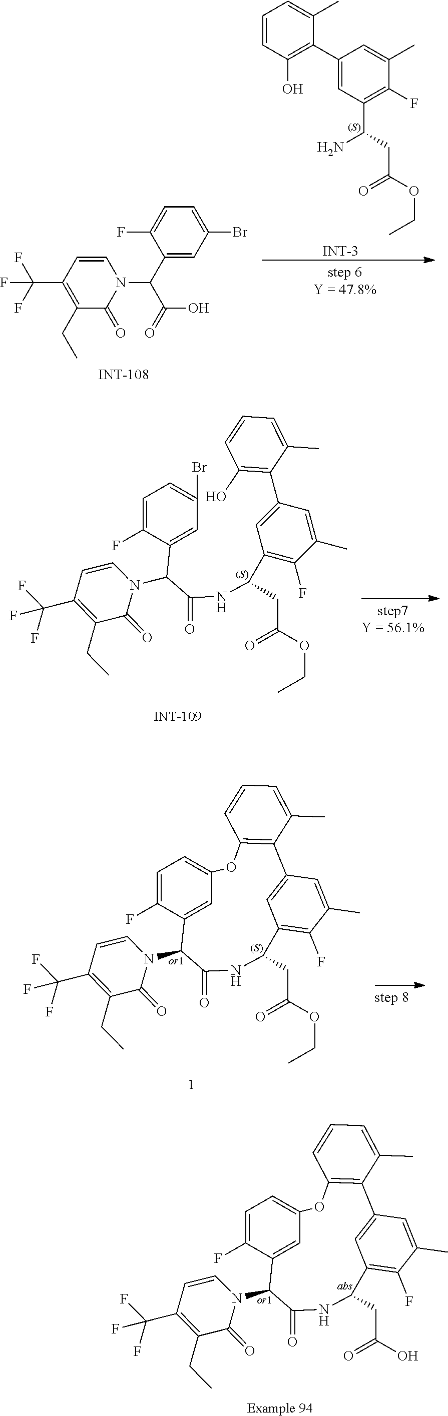

### Step 1: Preparation of ethyl (3S)-3-[2-(5-bromo-2-fluorophenyl)-2-[3-ethyl-2-oxo-4-(trifluoromethyl)pyridin-1-yl]acetamido]-3-{4-fluoro-2′-hydroxy-5,6′-dimethyl-[1,1′-biphenyl]-3-yl}propanoate

To a stirred mixture of (5-bromo-2-fluorophenyl)[3-ethyl-2-oxo-4-(trifluoromethyl)pyridin-1-yl]acetic acid (360 mg, 0.85 mmol, 1.00 equiv.) in MeCN (5 mL) was added HOBT (230 mg, 1.70 mmol, 2.00 equiv.), EDCI (326 mg, 1.70 mmol, 2.00 equiv.) at room temperature under nitrogen atmosphere. The resulting mixture was stirred for 1 h at room temperature under nitrogen atmosphere. To the above mixture was added ethyl (3S)-3-amino-3-{4-fluoro-2′-hydroxy-5,6′-dimethyl-[1,1′-biphenyl]-3-yl}propanoate (367 mg, 1.10 mmol, 1.03 equiv.) and Et₃N (517 mg, 5.11 mmol, 6.00 equiv.) in MeCN (5 mL) in portions over 1 min at room temperature. The resulting mixture was stirred for additional overnight at room temperature. The resulting mixture was concentrated under reduced pressure. The residue was purified by reversed-phase flash chromatography (C18 gel; mobile phase, MeCN in water (0.1% NH₃·H₂O), 40% to 60% gradient in 20 min; detector, UV 254/220 nm) to afford ethyl (3S)-3-[2-(5-bromo-2-fluorophenyl)-2-[3-ethyl-2-oxo-4-(trifluoromethyl)pyridin-1-yl]acetamido]-3-{4-fluoro-2′-hydroxy-5,6′-dimethyl-[1,1′-biphenyl]-3-yl}propanoate (300 mg, 47.8%, mixture) as colorless oil. LC-MS: (ES+H, m/z): [M+H]⁺=735.1.

### Step 2: Preparation of ethyl 2-[(14S,17S)-14-[3-ethyl-2-oxo-4-(trifluoromethyl)pyridin-1-yl]-12,19-difluoro-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (Intermediate 109)

To a stirred mixture of ethyl (3S)-3-[2-(5-bromo-2-fluorophenyl)-2-[3-ethyl-2-oxo-4-(trifluoromethyl)pyridin-1-yl]acetamido]-3-{4-fluoro-2′-hydroxy-5,6′-dimethyl-[1,1′-biphenyl]-3-yl}propanoate (80 mg, 0.10 mmol, 1.00 equiv.) in 1,4-dioxane (5 mL) were added CuI (10 mg, 0.05 mmol, 0.50 equiv.), K₂CO₃ (30 mg, 0.21 mmol, 2.00 equiv.) and (1R,2R)—N1,N2-dimethylcyclohexane-1,2-diamine (15 mg, 0.10 mmol, 1.00 equiv.) at room temperature under nitrogen atmosphere. The resulting mixture was stirred overnight at 100° C. under nitrogen atmosphere. The mixture was allowed to cool down to room temperature. The resulting mixture was concentrated under reduced pressure. The residue was purified by reversed-phase flash chromatography (C18 gel; mobile phase, MeCN in water (10 mmol/L NH₄HCO₃), 50% to 70% gradient in 20 min; detector, UV 254/220 nm) to afford ethyl 2-[(14S,17S)-14-[3-ethyl-2-oxo-4-(trifluoromethyl)pyridin-1-yl]-12,19-difluoro-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (40 mg, 56.1%, isomer 1) as a colorless oil. LC-MS: (ES+H, m/z): [M+H]⁺=655.2.

### Step 3: Preparation of [(14S,17S)-14-[3-ethyl-2-oxo-4-(trifluoromethyl)pyridin-1-yl]-12,19-difluoro-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid

To a stirred mixture of ethyl 2-[(14S,17S)-14-[3-ethyl-2-oxo-4-(trifluoromethyl)pyridin-1-yl]-12,19-difluoro-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (40 mg, 0.06 mmol, 1.00 equiv.) in THF (1 mL) was added LiOH·H₂O (5 mg, 0.12 mmol, 2.00 equiv.) in H₂O (0.5 mL) at room temperature under nitrogen atmosphere. The resulting mixture was stirred for 1 h at room temperature under nitrogen atmosphere. The resulting mixture was concentrated under reduced pressure. The crude product (40 mg) was purified by Prep-HPLC (Column: XBridge Shield RP18 OBD Column, 30*150 mm, 5 μm; Mobile Phase A: Water (10 mmol/L NH₄HCO₃), Mobile Phase B: MeCN; Flow rate: 60 mL/min; Gradient: 25% B to 45% B in 10 min; Wavelength: 254 nm/220 nm; RT1 (min): 9.1) to afford [(14S,17S)-14-[3-ethyl-2-oxo-4-(trifluoromethyl)pyridin-1-yl]-12,19-difluoro-3,20-dimethyl-15-oxo-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid (21.5 mg, 54.5%) as a white solid. LC-MS: (ES−H, m/z): [M−H]⁻=625.05. ¹H NMR (300 MHz, CD₃OD) δ 7.54 (ddd, J=9.0, 4.1, 2.9 Hz, 1H), 7.45 (dd, J=7.9, 1.2 Hz, 1H), 7.35 (t, J=7.8 Hz, 1H), 7.23-7.11 (m, 3H), 6.98 (d, J=6.4 Hz, 1H), 6.82 (s, 1H), 6.45 (m, 2H), 6.07 (dd, J=6.1, 3.0 Hz, 1H), 5.72 (brs, 1H), 2.75 (m, 4H), 2.36 (d, J=1.7 Hz, 3H), 2.02 (s, 3H), 1.18 (t, J=7.2 Hz, 3H). ¹⁹F NMR (282 MHz, CD₃OD) δ −63.25, −123.52, −126.69.

### Example 95

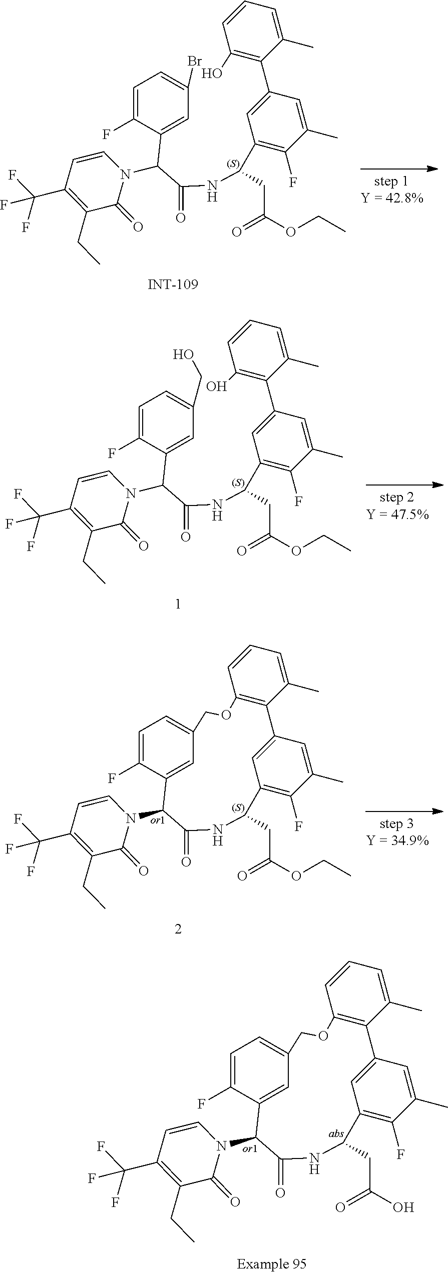

### Step 1: Preparation of ethyl (3S)-3-{2-[3-ethyl-2-oxo-4-(trifluoromethyl)pyridin-1-yl]-2-[2-fluoro-5-(hydroxymethyl)phenyl]acetamido}-3-{4-fluoro-2′-hydroxy-5,6′-dimethyl-[1,1′-biphenyl]-3-yl}propanoate

To a stirred mixture of ethyl (3S)-3-[2-(5-bromo-2-fluorophenyl)-2-[3-ethyl-2-oxo-4-(trifluoromethyl)pyridin-1-yl]acetamido]-3-{4-fluoro-2′-hydroxy-5,6′-dimethyl-[1,1′-biphenyl]-3-yl}propanoate (200 mg, 0.27 mmol, 1.00 equiv.) in 1,4-dioxane (8 mL) were added 2nd generation XPhos precatalyst (21 mg, 0.02 mmol, 0.10 equiv.) and (tributylstannyl)methanol (261 mg, 0.81 mmol, 3.00 equiv.) at room temperature under nitrogen atmosphere. The resulting mixture was stirred overnight at 90° C. under nitrogen atmosphere. The mixture was allowed to cool down to room temperature. The resulting mixture was diluted with ethyl acetate (10 mL). The residue was washed with water (2×10 mL). The combined organic layers were washed with brine (2×10 mL), dried over anhydrous Na₂SO₄, filtered, and concentrated. The residue was purified by reversed-phase flash chromatography (C18 gel; mobile phase, MeCN in water (10 mmol/L NH₄HCO₃), 40% to 60% gradient in 20 min; detector, UV 254/220 nm) to afford ethyl (3S)-3-{2-[3-ethyl-2-oxo-4-(trifluoromethyl)pyridin-1-yl]-2-[2-fluoro-5-(hydroxymethyl)phenyl]acetamido}-3-{4-fluoro-2′-hydroxy-5,6′-dimethyl-[1,1′-biphenyl]-3-yl}propanoate (80 mg, 42.8%, mixture) as a colorless oil. LC-MS: (ES+H, m/z): [M+H]⁺=687.3.

### Step 2: Preparation of ethyl 2-[(15S,18S)-15-[3-ethyl-2-oxo-4-(trifluoromethyl)pyridin-1-yl]-13,20-difluoro-3,21-dimethyl-16-oxo-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetate

To a stirred mixture of ethyl (3S)-3-{2-[3-ethyl-2-oxo-4-(trifluoromethyl)pyridin-1-yl]-2-[2-fluoro-5-(hydroxymethyl)phenyl]acetamido}-3-{4-fluoro-2′-hydroxy-5,6′-dimethyl-[1,1′-biphenyl]-3-yl}propanoate (80 mg, 0.12 mmol, 1.00 equiv.) in toluene (2 mL) was added 2-(tributylphosphanylidene)acetonitrile (141 mg, 0.59 mmol, 5.00 equiv.) at room temperature under nitrogen atmosphere. The resulting mixture was stirred overnight at 100° C. under nitrogen atmosphere. The mixture was allowed to cool down to room temperature. The resulting mixture was concentrated under reduced pressure. The residue was purified by reversed-phase flash chromatography (C18 gel; mobile phase, MeCN in water (10 mmol/L NH₄HCO₃), 40% to 60% gradient in 20 min; detector, UV 254/220 nm) to afford ethyl 2-[(15S,18S)-15-[3-ethyl-2-oxo-4-(trifluoromethyl)pyridin-1-yl]-13,20-difluoro-3,21-dimethyl-16-oxo-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetate (37 mg, 47.5%) as a yellow solid. LC-MS: (ES+H, m/z): [M+H]⁺=669.2. ¹H NMR (300 MHz, DMSO-d₆) δ 9.47 (d, J=7.6 Hz, 1H), 7.55 (d, J=6.3 Hz, 1H), 7.29 (d, J=8.3 Hz, 1H), 7.25-7.17 (m, 2H), 7.11 (d, J=7.4 Hz, 2H), 7.01 (d, J=14.5 Hz, 2H), 6.83 (d, J=7.4 Hz, 1H), 6.50 (d, J=6.9 Hz, 1H), 6.29 (d, J=7.5 Hz, 1H), 5.63-5.55 (m, 1H), 5.37 (d, J=13.2 Hz, 1H), 4.97 (d, J=13.1 Hz, 1H), 4.17-3.99 (m, 2H), 2.65 (t, J=7.9 Hz, 2H), 2.28 (s, 3H), 2.08 (s, 2H), 1.92 (s, 3H), 1.11 (dt, J=11.4, 7.2 Hz, 6H). ¹⁹F NMR (282 MHz, DMSO-d₆) δ, −60.78, −117.55, −126.89.

### Step 3: Preparation of [(15R*,18S)-15-[3-ethyl-2-oxo-4-(trifluoromethyl)pyridin-1-yl]-13,20-difluoro-3,21-dimethyl-16-oxo-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetic acid

To a stirred mixture of ethyl 2-[(15R*,18S)-15-[3-ethyl-2-oxo-4-(trifluoromethyl)pyridin-1-yl]-13,20-difluoro-3,21-dimethyl-16-oxo-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetate (30 mg, 0.05 mmol, 1.00 equiv.) in THF (1 mL) and H₂O (0.2 mL) was added LiOH·H₂O (3 mg, 0.14 mmol, 3.00 equiv.) at room temperature. The resulting mixture was stirred for 4 h at room temperature under nitrogen atmosphere. The resulting mixture was concentrated under reduced pressure. The crude product (30 mg) was purified by Prep-HPLC (Column: Xselect CSH Fluoro-Phenyl Column, 30*150 mm, 5 μm; Mobile Phase A: Water (0.1% HCOOH), Mobile Phase B: MeCN; Flow rate: 60 mL/min; Gradient: 32% B to 52% B in 10 min; Wavelength: 254 nm/220 nm; RT1 (min): 8.17) to afford [(15R*,18S)-15-[3-ethyl-2-oxo-4-(trifluoromethyl)pyridin-1-yl]-13,20-difluoro-3,21-dimethyl-16-oxo-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetic acid (10.2 mg, 34.9%) as a white solid. LC-MS: (ES+H, m/z): [M+H]⁺=641.15. ¹H NMR (300 MHz, DMSO-d₆) δ 9.46 (d, J=7.3 Hz, 1H), 7.52 (ddd, J=7.7, 5.0, 2.0 Hz, 1H), 7.28 (d, J=8.3 Hz, 1H), 7.25-7.08 (m, 4H), 7.06-6.93 (m, 2H), 6.83 (d, J=7.4 Hz, 1H), 6.49 (dd, J=6.8, 2.2 Hz, 1H), 6.29 (d, J=7.4 Hz, 1H), 5.56 (td, J=8.5, 5.0 Hz, 1H), 5.36 (d, J=13.2 Hz, 1H), 4.97 (d, J=13.2 Hz, 1H), 2.62 (ddd, J=17.4, 12.1, 6.9 Hz, 4H), 2.28 (d, J=1.9 Hz, 3H), 1.91 (s, 3H), 1.10 (t, J=7.2 Hz, 3H). ¹⁹F NMR (282 MHz, DMSO-d₆) δ −60.78, −117.20, −127.23.

### Example 96

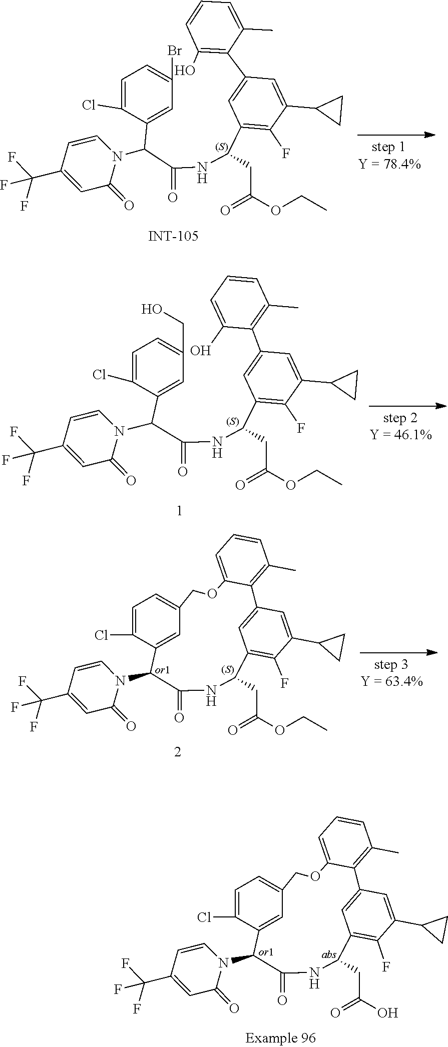

### Step 1: Preparation of ethyl (3S)-3-{2-[2-chloro-5-(hydroxymethyl)phenyl]-2-[2-oxo-4-(trifluoromethyl)pyridin-1-yl]acetamido}-3-{5-cyclopropyl-4-fluoro-2′-hydroxy-6′-methyl-[1,1′-biphenyl]-3-yl}propanoate

To a stirred solution of ethyl (3S)-3-[2-(5-bromo-2-chlorophenyl)-2-[2-oxo-4-(trifluoromethyl)pyridin-1-yl]acetamido]-3-{5-cyclopropyl-4-fluoro-2′-hydroxy-6′-methyl-[1,1′-biphenyl]-3-yl}propanoate (1.50 g, 2.00 mmol, 1.00 equiv.) and (tributylstannyl)methanol (1.93 g, 6.00 mmol, 3.00 equiv.) in 1,4-dioxane (25 mL) was added 2nd generation XPhos precatalyst/X-Phos aminobiphenyl palladium chloride precatalyst (157 mg, 0.20 mmol, 0.10 equiv.). The resulting mixture was stirred overnight at 90° C. under nitrogen atmosphere. The mixture was allowed to cool down to room temperature. The resulting mixture was filtered, the filter cake was washed with EtOAc (100 mL). The filtrate was concentrated under reduced pressure. The residue was purified by reversed-phase flash chromatography (C18 gel; mobile phase, CH₃CN in water (10 mmol/L NH₄HCO₃), 40% to 70% gradient in 20 min; detector, UV 254 nm) to afford ethyl (3S)-3-{2-[2-chloro-5-(hydroxymethyl)phenyl]-2-[2-oxo-4-(trifluoromethyl)pyridin-1-yl]acetamido}-3-{5-cyclopropyl-4-fluoro-2′-hydroxy-6′-methyl-[1,1′-biphenyl]-3-yl}propanoate (1.10 g, 78.4%) as a white solid. LC-MS: (ES+H, m/z): [M+H]⁺=701.15

### Step 2: Preparation of ethyl 2-[(15S,18S)-13-chloro-21-cyclopropyl-20-fluoro-3-methyl-16-oxo-15-[2-oxo-4-(trifluoromethyl)pyridin-1-yl]-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetate

To mixture of ethyl (3S)-3-{2-[2-chloro-5-(hydroxymethyl)phenyl]-2-[2-oxo-4-(trifluoromethyl)pyridin-1-yl]acetamido}-3-{5-cyclopropyl-4-fluoro-2′-hydroxy-6′-methyl-[1,1′-biphenyl]-3-yl}propanoate (1.00 g, 1.42 mmol, 1.00 equiv.) and 2-(tributylphosphanylidene)acetonitrile (1.72 g, 7.13 mmol, 5.00 equiv.) in toluene (50 mL). After stirring overnight at 100° C. under a nitrogen atmosphere. The mixture was allowed to cool down to room temperature. The resulting mixture was diluted with EtOAc (100 mL). The organic layers were washed with water (2×20 mL), dried over anhydrous Na₂SO₄, filtered, and concentrated. The residue was purified by reversed-phase flash chromatography (C18 gel; mobile phase, CH₃CN in water (0.1% NH₃·H₂O), 40% to 70% gradient in 20 min; detector, UV 220 nm) to provide ethyl 2-[(15S,18S)-13-chloro-21-cyclopropyl-20-fluoro-3-methyl-16-oxo-15-[2-oxo-4-(trifluoromethyl)pyridin-1-yl]-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetate (450 mg, 46.1%) as an off-white solid. LC-MS: (ES+H, m/z): [M+H]⁺=683.15

### Step 3: Preparation of [(15S,18S)-13-chloro-21-cyclopropyl-20-fluoro-3-methyl-16-oxo-15-[2-oxo-4-(trifluoromethyl)pyridin-1-yl]-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetic acid

To a stirred mixture of ethyl 2-[(15S,18S)-13-chloro-21-cyclopropyl-20-fluoro-3-methyl-16-oxo-15-[2-oxo-4-(trifluoromethyl)pyridin-1-yl]-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetate (420 mg, 0.61 mmol, 1.00 equiv.) in THF (12 mL) was added LiOH·H₂O (51 mg, 1.23 mmol, 2.00 equiv.) in water (6 mL) at room temperature under nitrogen atmosphere. The resulting mixture was stirred for 1 h at room temperature under nitrogen atmosphere. The resulting mixture was diluted with water (6 mL). The mixture was acidified to pH 5 with 1 M HCl (aq.). The resulting mixture was concentrated under vacuum. The crude product (410 mg) was purified by Prep-HPLC (Column: XBridge Shield RP18 OBD Column, 30*250 mm, 5 μm; Mobile Phase A: Water (10 mmol/L NH₄HCO₃), Mobile Phase B: ACN; Flow rate: 60 mL/min; Gradient: 32% B to 42% B in 10 min; Wavelength: 254 nm/220 nm; RT1 (min): 9.1) to afford [(15S,18S)-13-chloro-21-cyclopropyl-20-fluoro-3-methyl-16-oxo-15-[2-oxo-4-(trifluoromethyl)pyridin-1-yl]-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetic acid (255.7 mg, 63.4%) as a white solid. LC-MS: (ES+H, m/z): [M+H]⁺=655.10. ¹H NMR (400 MHz, DMSO-d₆) δ 12.46 (s, 1H), 9.49 (d, J=7.2 Hz, 1H), 7.58-7.48 (m, 2H), 7.28 (d, J=8.4 Hz, 1H), 7.24-7.12 (m, 3H), 6.91 (s, 1H), 6.87-6.80 (m, 2H), 6.65 (dd, J=6.8, 2.1 Hz, 1H), 6.45 (d, J=6.6 Hz, 1H), 6.34 (dd, J=7.4, 2.2 Hz, 1H), 5.59-5.54 (m, 1H), 5.38 (d, J=13.3 Hz, 1H), 4.99 (d, J=13.4 Hz, 1H), 2.66 (dd, J=16.2, 4.3 Hz, 1H), 2.60-2.53 (m, 1H), 2.13-2.04 (m, 1H), 1.89 (s, 3H), 1.04-0.93 (m, 2H), 0.80-0.69 (m, 2H). ¹⁹F NMR (400 MHz, DMSO-d₆) δ −65.35, −130.59.

### Example 97

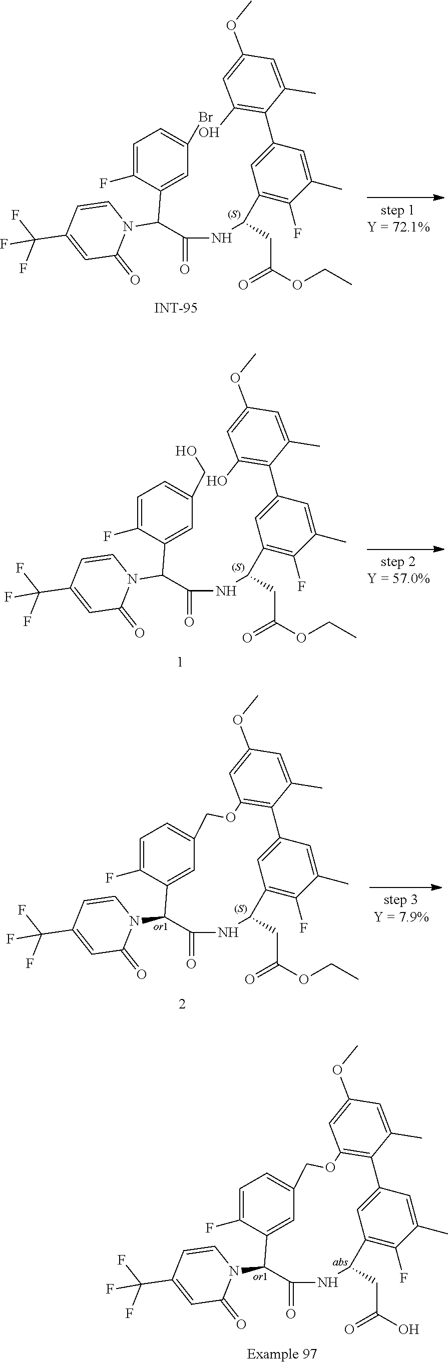

### Step 1: Preparation of ethyl (3S)-3-{4-fluoro-2′-hydroxy-4′-methoxy-5,6′-dimethyl-[1,1′-biphenyl]-3-yl}-3-{2-[2-fluoro-5-(hydroxymethyl)phenyl]-2-[2-oxo-4-(trifluoromethyl)pyridin-1-yl]acetamido}propanoate

To a stirred solution of ethyl (3S)-3-[2-(5-bromo-2-fluorophenyl)-2-[2-oxo-4-(trifluoromethyl)pyridin-1-yl]acetamido]-3-{4-fluoro-2′-hydroxy-4′-methoxy-5,6′-dimethyl-[1,1′-biphenyl]-3-yl}propanoate (190 mg, 0.25 mmol, 1.00 equiv.) and (tributylstannyl)methanol (248 mg, 0.77 mmol, 3.00 equiv.) in 1,4-dioxane (6 mL) was added 2nd generation XPhos precatalyst (20 mg, 0.02 mmol, 0.10 equiv.). The resulting mixture was stirred overnight at 90° C. under nitrogen atmosphere. The mixture was allowed to cool down to room temperature. The resulting mixture was filtered, the filter cake was washed with EtOAc (100 mL). The filtrate was concentrated under reduced pressure. The residue was purified by reversed-phase flash chromatography (C18 gel; mobile phase, CH₃CN in water (10 mmol/L NH₄HCO₃), 40% to 70% gradient in 20 min; detector, UV 254 nm) to afford ethyl (3S)-3-{4-fluoro-2′-hydroxy-4′-methoxy-5,6′-dimethyl-[1,1′-biphenyl]-3-yl}-3-{2-[2-fluoro-5-(hydroxymethyl)phenyl]-2-[2-oxo-4-(trifluoromethyl)pyridin-1-yl]acetamido}propanoate (128 mg, 72.1%, mixture) as a white solid. LC-MS: (ES+H, m/z): [M+H]⁺=689.30.

### Step 2: Preparation of ethyl 2-[(15S,18S)-13,20-difluoro-5-methoxy-3,21-dimethyl-16-oxo-15-[2-oxo-4-(trifluoromethyl)pyridin-1-yl]-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetate

To a stirred solution of ethyl (3S)-3-{4-fluoro-2′-hydroxy-4′-methoxy-5,6′-dimethyl-[1,1′-biphenyl]-3-yl}-3-{2-[2-fluoro-5-(hydroxymethyl)phenyl]-2-[2-oxo-4-(trifluoromethyl)pyridin-1-yl]acetamido}propanoate (100 mg, 0.13 mmol, 1.00 equiv.) in toluene (6 mL) was added CMBP (157 mg, 0.65 mmol, 5.00 equiv.). The resulting mixture was stirred for 3 h at 100° C. under nitrogen atmosphere. The mixture was allowed to cool down to room temperature. The resulting mixture was diluted with EtOAc (100 mL). The organic layers were washed with water (2×20 mL), dried over anhydrous Na₂SO₄, filtered, and concentrated. The residue was purified by reversed-phase flash chromatography (C18 gel; mobile phase, CH₃CN in water (0.1% NH₃·H₂O), 40% to 70% gradient in 20 min; detector, UV 220 nm) to provide ethyl 2-[(15S,18S)-13,20-difluoro-5-methoxy-3,21-dimethyl-16-oxo-15-[2-oxo-4-(trifluoromethyl)pyridin-1-yl]-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetate (50 mg, 57.0%) as an white solid. LC-MS: (ES+H, m/z): [M+H]⁺=671.15.

### Step 3: Preparation of [(15S,18S)-13,20-difluoro-5-methoxy-3,21-dimethyl-16-oxo-15-[2-oxo-4-(trifluoromethyl)pyridin-1-yl]-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetic acid

To a stirred mixture of ethyl 2-[(15S,18S)-13,20-difluoro-5-methoxy-3,21-dimethyl-16-oxo-15-[2-oxo-4-(trifluoromethyl)pyridin-1-yl]-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetate (50 mg, 0.07 mmol, 1.00 equiv.) in THF (1 mL) was added LiOH·H₂O (3 mg, 0.15 mmol, 2.00 equiv.) in water (0.5 mL). The resulting mixture was stirred for 1 h at room temperature under nitrogen atmosphere. The resulting mixture was diluted with water (2 mL). The mixture was acidified to pH 5 with 1 M HCl (aq.). The resulting mixture was concentrated under vacuum. The crude product (40 mg) was purified by Prep-HPLC (Column: XBridge Prep OBD C18 Column, 30*150 mm, 10 μm; Mobile Phase A: Water (10 mmol/L NH₄HCO₃+0.05% NH₃H₂O), Mobile Phase B: MeCN; Flow rate: 60 mL/min; Gradient: 29% B to 45% B in 8 min; Wavelength: 254 nm/220 nm; RT1 (min): 9.57) to afford [(15S,18S)-13,20-difluoro-5-methoxy-3,21-dimethyl-16-oxo-15-[2-oxo-4-(trifluoromethyl)pyridin-1-yl]-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetic acid (3.8 mg, 7.9%) as a white solid. LC-MS: (ES+H, m/z): [M+H]⁺=643.10. ¹H NMR (400 MHz, DMSO-d₆) δ 9.65 (s, 1H), 7.56-7.53 (m, 1H), 7.31 (d, J=7.3 Hz, 1H), 7.21 (dd, J=10.0, 8.3 Hz, 1H), 7.12 (dd, J=7.8, 2.1 Hz, 1H), 7.01 (s, 1H), 6.94 (dd, J=6.9, 2.0 Hz, 1H), 6.85 (d, J=2.2 Hz, 2H), 6.48-6.40 (m, 2H), 6.35 (dd, J=7.3, 2.2 Hz, 1H), 5.50 (s, 1H), 5.37 (d, J=13.3 Hz, 1H), 4.94 (d, J=13.2 Hz, 1H), 3.76 (d, J=1.9 Hz, 3H), 2.49-2.40 (m, 2H), 2.26 (s, 3H), 1.87 (s, 3H). ¹⁹F NMR (400 MHz, DMSO-d₆) δ −65.408, −117.321, −127.715.

### Example 98

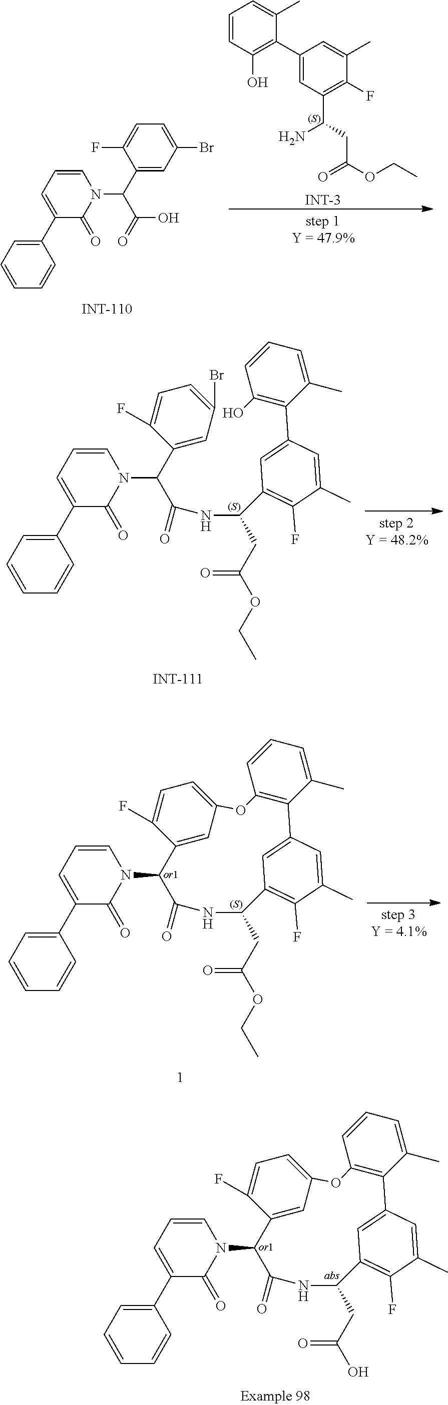

### Step 1: Preparation of ethyl (3S)-3-(2-(5-bromo-2-fluorophenyl)-2-(2-oxo-3-phenylpyridin-1(2H)-yl)acetamido)-3-(4-fluoro-2′-hydroxy-5,6′-dimethyl-[1,1′-biphenyl]-3-yl)propanoate

To a stirred solution of (5-bromo-2-fluorophenyl)(2-oxo-3-phenylpyridin-1-yl)acetic acid (280 mg, 0.70 mmol, 1.00 equiv.) and ethyl (3S)-3-amino-3-{4-fluoro-2′-hydroxy-5,6′-dimethyl-[1,1′-biphenyl]-3-yl}propanoate (206 mg, 0.62 mmol, 2.50 equiv.) in ACN (6 mL) were added TCFH (586 mg, 2.09 mmol, 3.00 equiv.) and NMI (343 mg, 4.18 mmol, 6.00 equiv.) at 25° C. The resulting mixture was stirred for 16 h at 80° C. under nitrogen atmosphere. The mixture was allowed to cool down to 25° C. The residue was purified by reversed-phase flash chromatography (C18 gel; mobile phase, MeCN in water (0.1% NH₃·H₂O), 60% to 100% gradient in 20 min; detector, UV 254 nm) to afford ethyl (3S)-3-[2-(5-bromo-2-fluorophenyl)-2-(2-oxo-3-phenylpyridin-1-yl)acetamido]-3-{4-fluoro-2′-hydroxy-5,6′-dimethyl-[1,1′-biphenyl]-3-yl}propanoate (250 mg, 47.9%, mixture) as a white solid. LC-MS: (ES+H, m/z): [M+H]⁺=715.00/717.00. ¹H NMR (400 MHz, DMSO-d₆) δ 9.36 (dd, J=17.7, 8.3 Hz, 1H), 9.08 (s, 1H), 7.78-7.44 (m, 5H), 7.42-7.28 (m, 3H), 7.28-7.11 (m, 2H), 7.06-6.91 (m, 4H), 6.79-6.59 (m, 2H), 6.28 (dt, J=23.3, 7.0 Hz, 1H), 5.74-5.54 (m, 1H), 4.03 (dq, J=14.9, 7.4 Hz, 2H), 2.91-2.62 (m, 2H), 2.25 (d, J=11.2 Hz, 3H), 1.87 (s, 3H), 1.08 (dt, J=14.1, 7.1 Hz, 3H). ¹⁹F NMR (377 MHz, DMSO-d₆) δ −117.02, 127.02.

### Step 2: Preparation of ethyl 2-[(14S,17S)-12,19-difluoro-3,20-dimethyl-15-oxo-14-(2-oxo-3-phenylpyridin-1-yl)-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (Intermediate 111)

To a stirred solution of ethyl (3S)-3-[2-(5-bromo-2-fluorophenyl)-2-(2-oxo-3-phenylpyridin-1-yl)acetamido]-3-{4-fluoro-2′-hydroxy-5,6′-dimethyl-[1,1′-biphenyl]-3-yl}propanoate (100 mg, 0.14 mmol, 1.00 equiv.) and (1R,2R)—N1,N2-dimethylcyclohexane-1,2-diamine (20 mg, 0.14 mmol, 1.00 equiv.) in dioxane (2 mL) were added CuI (13 mg, 0.07 mmol, 0.50 equiv.) and K₂CO₃ (39 mg, 0.28 mmol, 2.00 equiv.) at 25° C. The resulting mixture was stirred for 2 h at 100° C. under nitrogen atmosphere. The resulting mixture was diluted with water (50 mL). The resulting mixture was extracted with EtOAc (3×50 mL). The combined organic layers were washed with brine (100 mL), dried over anhydrous Na₂SO₄, filtered, and concentrated. The crude product (150 mg) was purified by Prep-HPLC (Column: XSelect CSH Prep C18 OBD Column, 30*150 mm, 5 μm; Mobile Phase A: Water (0.10% HCOOH, Mobile Phase B: MeCN; Flow rate: 60 mL/min; Gradient: 60% B to 80% B in 10 min; Wavelength: 254 nm/220 nm; RT1 (min): 9.87) to afford ethyl 2-[(14S,17S)-12,19-difluoro-3,20-dimethyl-15-oxo-14-(2-oxo-3-phenylpyridin-1-yl)-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (45 mg, 48.2%) as a white solid. LC-MS: (ES+H, m/z): [M+H]⁺=635.3.

### Step 3: Preparation of [(14S,17S)-12,19-difluoro-3,20-dimethyl-15-oxo-14-(2-oxo-3-phenylpyridin-1-yl)-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid

A mixture of ethyl 2-[(14S,17S)-12,19-difluoro-3,20-dimethyl-15-oxo-14-(2-oxo-3-phenylpyridin-1-yl)-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (40 mg, 0.063 mmol, 1.00 equiv.) and LiOH·H₂O (13 mg, 0.32 mmol, 5.00 equiv.) in THF (4 mL) and H₂O (1 mL) was stirred for 2 h at 30° C. under nitrogen atmosphere. The resulting mixture was concentrated under reduced pressure. The crude product was purified by reverse phase flash (Column: XBridge Shield RP18 OBD Column 30*150 mm, 5 μm; Mobile Phase A: Water (0.1% HCOOH), Mobile Phase B: MeCN; Flow rate: 60 mL/min; Gradient: 51% B to 71% B in 8 min; Wavelength: 254 nm/220 nm; RT1 (min): 8.92) to afford [(14S,17S)-12,19-difluoro-3,20-dimethyl-15-oxo-14-(2-oxo-3-phenylpyridin-1-yl)-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid (1.6 mg, 4.14%) as an off-white solid. LC-MS: (ES+H, m/z): [M+H]⁺=607.15. ¹H NMR (400 MHz, DMSO-d₆) δ 9.26 (s, 1H), 7.69 (d, J=7.6 Hz, 2H), 7.59 (q, J=10.3, 8.7 Hz, 3H), 7.37 (dq, J=24.5, 7.5 Hz, 4H), 7.28-7.17 (m, 2H), 7.07 (d, J=6.9 Hz, 1H), 7.00 (d, J=6.6 Hz, 1H), 6.85 (s, 1H), 6.45 (d, J=6.1 Hz, 1H), 6.24 (t, J=7.0 Hz, 1H), 5.98 (d, J=5.8 Hz, 1H), 5.50 (s, 1H), 2.55-2.51 (m, 2H), 2.31 (s, 3H), 1.96 (s, 3H). ¹⁹F NMR (376 MHz, DMSO-d₆) δ −121.67, −125.94.

### Example 99

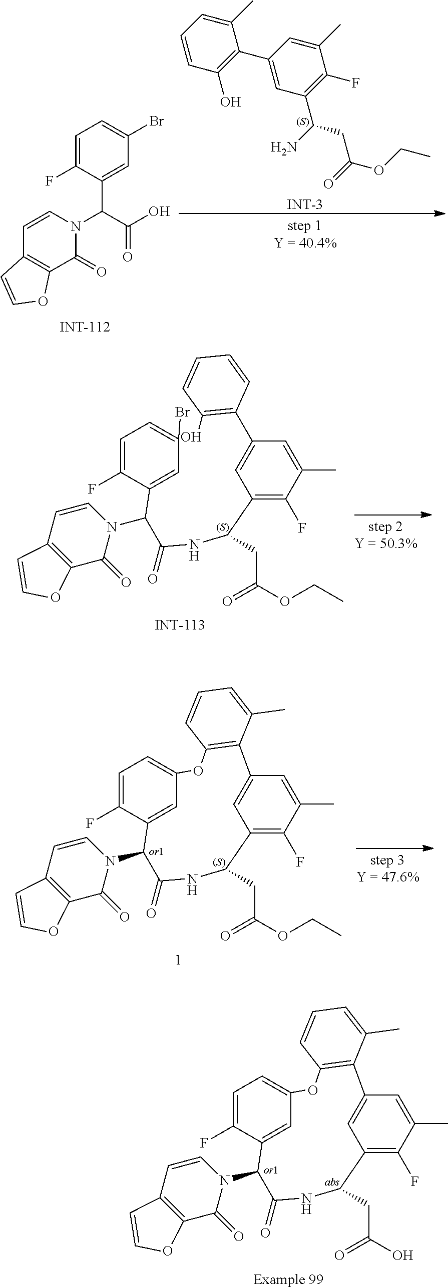

### Step 1: Preparation of ethyl (3S)-3-(2-(5-bromo-2-fluorophenyl)-2-(7-oxofuro[2,3-c]pyridin-6(7H)-yl)acetamido)-3-(4-fluoro-2′-hydroxy-5,6′-dimethyl-[1,1′-biphenyl]-3-yl)propanoate

A mixture (1) of 2-(5-bromo-2-fluorophenyl)-2-(7-oxofuro[2,3-c]pyridin-6(7H)-yl)acetic acid (200 mg, 0.546 mmol, 1.00 equiv.), HATU (312 mg, 0.82 mmol, 1.50 equiv.) and DIEA (318 mg, 2.46 mmol, 3.00 equiv.) in CH₂Cl₂ (10 mL) was stirred for 30 min at 25° C. under nitrogen atmosphere. The mixture (2) of ethyl (S)-3-amino-3-(4-fluoro-2′-hydroxy-5,6′-dimethyl-[1,1′-biphenyl]-3-yl)propanoate (254 mg, 0.764 mmol, 1.40 equiv.) and TEA (332 mg, 3.276 mmol, 6.00 equiv.) in CH₂Cl₂ (10 mL) was stirred for 1 h at 30° C. under nitrogen atmosphere then concentrated under reduced pressure. The mixture (2) was diluted with CH₂Cl₂ (10 mL) then added dropwise into the mixture (1) at 0° C. under nitrogen atmosphere. The resulting mixture was stirred for 2 h at 25° C. under nitrogen atmosphere. The resulting mixture was concentrated under reduced pressure. The residue was purified by reverse phase flash (C18 gel; mobile phase, MeCN in water (0.1% NH₃·H₂O), 20% to 70% gradient in 30 min, hold at 65%; detector, UV 254/220 nm) to afford ethyl (3S)-3-(2-(5-bromo-2-fluorophenyl)-2-(7-oxofuro[2,3-c]pyridin-6(7H)-yl)acetamido)-3-(4-fluoro-2′-hydroxy-5,6′-dimethyl-[1,1′-biphenyl]-3-yl)propanoate (150 mg, 40.4%, mixture) as a white solid. LC-MS: (ES+H, m/z): [M+H]⁺=679.00. ¹H NMR (400 MHz, DMSO-d₆) δ 9.40-9.28 (m, 1H), 9.14-9.05 (m, 1H), 8.16 (dd, J=11.0, 1.9 Hz, 1H), 7.74-7.58 (m, 1H), 7.48-7.12 (m, 2H), 7.07-6.93 (m, 5H), 6.88 (dd, J=19.3, 2.0 Hz, 1H), 6.72 (dt, 2H), 6.52 (dd, J=33.1, 7.3 Hz, 1H), 5.76-5.50 (m, 1H), 4.16-3.90 (m, 2H), 2.92-2.60 (m, 2H), 2.25 (d, J=12.6 Hz, 3H), 1.89 (s, 3H), 1.08 (dt, J=22.4, 7.1 Hz, 3H). ¹⁹F NMR (377 MHz, DMSO-d₆) δ −117.15.

### Step 2: Preparation of ethyl 2-[(14R*,17S)-12,19-difluoro-3,20-dimethyl-15-oxo-14-{7-oxofuro[2,3-c]pyridin-6-yl}-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (Intermediate 113)

To a stirred mixture of ethyl (3S)-3-(2-(5-bromo-2-fluorophenyl)-2-(7-oxofuro[2,3-c]pyridin-6(7H)-yl)acetamido)-3-(4-fluoro-2′-hydroxy-5,6′-dimethyl-[1,1′-biphenyl]-3-yl)propanoate (100 mg, 0.15 mmol, 1.00 equiv.) and K₂CO₃ (61 mg, 0.44 mmol, 3.00 equiv.) in 1,4-dioxane (4 mL) were added CuI (14 mg, 0.073 mmol, 0.50 equiv.) and (1R,2R)-1-N,2-N-dimethylcyclohexane-1,2-diamine (21 mg, 0.15 mmol, 1.00 equiv.) at 25° C. The resulting mixture was stirred for 4 h at 100° C. under nitrogen atmosphere. The mixture was allowed to cool down to 25° C. The resulting mixture was filtered, and the filter cake was washed with MeCN (3×5 mL). The filtrate was concentrated under reduced pressure. The residue was purified by Prep-HPLC (Column: Xselect CSH C18 OBD Column 30*150 mm, 5 μm; Mobile Phase A: Water (0.1% HCOOH), Mobile Phase B: MeCN; Flow rate: 60 mL/min; Gradient: 54% B to 74% B in 8 min; Wavelength: 254 nm/220 nm; RT1 (min): 8.12) to afford ethyl 2-[(14R*,17S)-12,19-difluoro-3,20-dimethyl-15-oxo-14-{7-oxofuro[2,3-c]pyridin-6-yl}-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (45 mg, 50.9%) as a white solid. LC-MS: (ES+H, m/z): [M+H]⁺=599.20.

### Step 3: Preparation of [(14R*,17S)-12,19-difluoro-3,20-dimethyl-15-oxo-14-{7-oxofuro[2,3-c]pyridin-6-yl}-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid

To a stirred mixture of ethyl 2-[(14R*,17S)-12,19-difluoro-3,20-dimethyl-15-oxo-14-{7-oxofuro[2,3-c]pyridin-6-yl}-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (45 mg, 0.075 mmol, 1.00 equiv.) in THF (2 mL) was added LiOH (5 mg, 0.23 mmol, 3.00 equiv.) in H₂O (0.4 mL) at 25° C. The resulting mixture was stirred for 2.5 h at 25° C. under nitrogen atmosphere. The resulting mixture was concentrated under vacuum. The crude product was purified by Prep-HPLC (Column: XSelect CSH Prep C18 OBD Column, 30*150 mm, 5 μm; Mobile Phase A: Water (0.1% HCOOH), Mobile Phase B: MeCN; Flow rate: 60 mL/min; Gradient: 38% B to 58% B in 10 min; Wavelength: 254 nm/220 nm; RT1 (min): 10.6), the pure factions were concentrated under vacuum then lyophilized to afford [(14R*,17S)-12,19-difluoro-3,20-dimethyl-15-oxo-14-{7-oxofuro[2,3-c]pyridin-6-yl}-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid (20.6 mg, 47.6%) as a white solid. LC-MS: (ES+H, m/z): [M+H]⁺=571.25. ¹H NMR (300 MHz, DMSO-d₆) δ 12.46 (s, 1H), 9.14 (d, J=7.4 Hz, 1H), 8.16 (d, J=2.0 Hz, 1H), 7.66-7.49 (m, 2H), 7.37 (t, J=7.9 Hz, 1H), 7.27-7.15 (m, 2H), 7.08-7.00 (m, 1H), 6.95-6.81 (m, 3H), 6.54-6.41 (m, 2H), 5.95 (dd, J=6.3, 2.9 Hz, 1H), 5.56-5.40 (m, 1H), 2.82-2.62 (m, 2H), 2.31 (s, 3H), 1.96 (s, 3H). ¹⁹F NMR (282 MHz, DMSO-d₆) δ −121.847, −125.881.

### Example 100

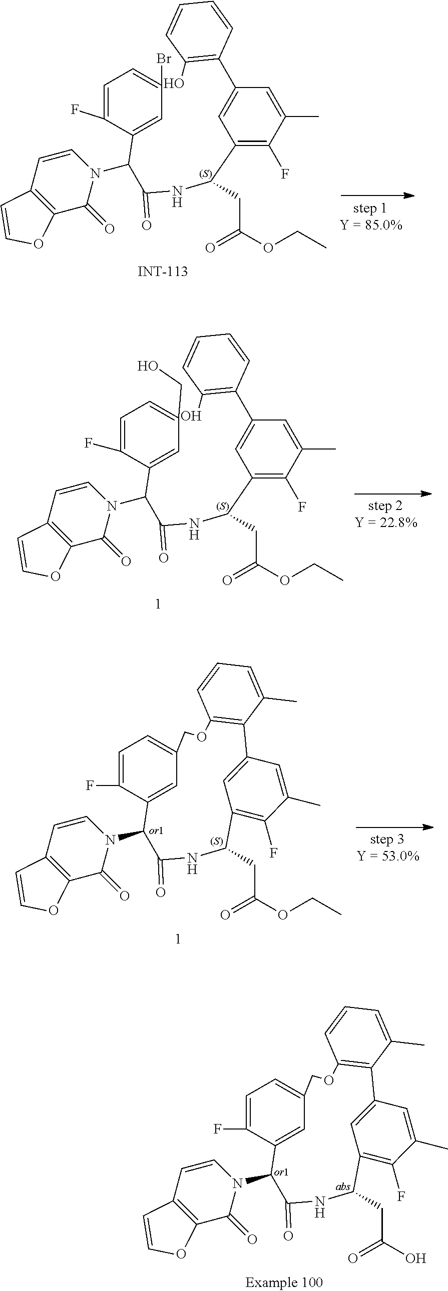

### Step 1: Preparation of ethyl (3S)-3-(4-fluoro-2′-hydroxy-5,6′-dimethyl-[1,1′-biphenyl]-3-yl)-3-(2-(2-fluoro-5-(hydroxymethyl)phenyl)-2-(7-oxofuro[2,3-c]pyridin-6(7H)-yl)acetamido)propanoate

To a stirred mixture of ethyl (3S)-3-(2-(5-bromo-2-fluorophenyl)-2-(7-oxofuro[2,3-c]pyridin-6(7H)-yl)acetamido)-3-(4-fluoro-2′-hydroxy-5,6′-dimethyl-[1,1′-biphenyl]-3-yl)propanoate (260 mg, 0.38 mmol, 1.00 equiv.) and (tributylstannyl)methanol (369 mg, 1.15 mmol, 3.00 equiv.) in 1,4-dioxane (15 mL) was added 2nd generation XPhos precatalyst (30 mg, 0.04 mmol, 0.10 equiv.). The resulting mixture was stirred for 16 h at 50° C. under nitrogen atmosphere. The mixture was allowed to cool down to 25° C. The reaction was quenched by the addition of 10% KF (aq.) (100 mL) and stirred for 30 min at 25° C. The resulting mixture was extracted with EtOAc (3×50 mL). The combined organic layers were washed with brine (3×50 mL), dried over anhydrous Na₂SO₄, filtered, and concentrated. The residue was purified by reverse phase flash (C18 gel; mobile phase, MeCN in water (0.1% NH₃·H₂O), 30% to 80% gradient in 20 min; detector, UV 220 nm) to afford ethyl (3S)-3-(4-fluoro-2′-hydroxy-5,6′-dimethyl-[1,1′-biphenyl]-3-yl)-3-(2-(2-fluoro-5-(hydroxymethyl)phenyl)-2-(7-oxofuro[2,3-c]pyridin-6(7H)-yl)acetamido)propanoate (180 mg, 85.0%) as a white solid. LC-MS: (ES+H, m/z): [M+H]⁺=631.15. 1H NMR (400 MHz, DMSO-d₆) δ 9.34-9.15 (m, 1H), 9.13-9.00 (m, 1H), 8.22-8.00 (m, 1H), 7.50-7.25 (m, 2H), 7.24-7.09 (m, 1H), 7.07-6.92 (m, 5H), 6.87 (dd, J=17.6, 2.0 Hz, 1H), 6.79-6.66 (m, 2H), 6.50 (dd, J=30.0, 7.2 Hz, 1H), 5.73-5.55 (m, 1H), 5.22 (dt, J=52.0, 5.7 Hz, 1H), 4.61-4.44 (m, 1H), 4.41-4.17 (m, 1H), 4.06-3.90 (m, 2H), 2.88-2.64 (m, 2H), 2.31-2.15 (m, 3H), 1.98-1.78 (m, 3H), 1.06 (dt, J=8.2, 7.1 Hz, 3H).

### Step 2: Preparation of ethyl 2-[(15S,18S)-13,20-difluoro-3,21-dimethyl-16-oxo-15-{7-oxofuro[2,3-c]pyridin-6-yl}-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetate

To a stirred solution of ethyl (3S)-3-{4-fluoro-2′-hydroxy-5,6′-dimethyl-[1,1′-biphenyl]-3-yl}-3-{2-[2-fluoro-5-(hydroxymethyl)phenyl]-2-{7-oxofuro[2,3-c]pyridin-6-yl}acetamido}propanoate (180 mg, 0.29 mmol, 1.00 equiv.) in toluene (10 mL) was added 2-(tributyl-lambda5-phosphanylidene)acetonitrile (344 mg, 1.43 mmol, 5.00 equiv.). The resulting mixture was stirred for 2 h at 100° C. under nitrogen atmosphere. The mixture was allowed to cool down to room temperature. The resulting mixture was concentrated under reduced pressure. The residue was purified by reversed-phase flash chromatography (C18 gel; mobile phase, MeCN in water (0.1% NH₃·H₂O), 30% to 70% gradient in 30 min; detector, UV 220 nm) to provide ethyl 2-[(15S,18S)-13,20-difluoro-3,21-dimethyl-16-oxo-15-{7-oxofuro[2,3-c]pyridin-6-yl}-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetate (40 mg, 22.8%) as a white solid. LC-MS: (ES+H, m/z): [M+H]⁺=613.3

### Step 3: Preparation of [(15R*,18S)-13,20-difluoro-3,21-dimethyl-16-oxo-15-{7-oxofuro[2,3-c]pyridin-6-yl}-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetic acid

A mixture of ethyl 2-[(15S,18S)-13,20-difluoro-3,21-dimethyl-16-oxo-15-{7-oxofuro[2,3-c]pyridin-6-yl}-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetate (35 mg, 0.06 mmol, 1.00 equiv.) and LiOH·H₂O (5 mg, 0.23 mmol, 4.00 equiv.) in THF (2 mL) and H₂O (0.5 mL) was stirred for 4 h at room temperature under nitrogen atmosphere. The mixture was acidified to pH 5 with HCl (aq. 3 N). The crude product was purified by Prep-HPLC (Column: XBridge Prep OBD C18 Column, 30*150 mm, 10 μm; Mobile Phase A: Water (10 mmol/L NH₄HCO₃+0.05% NH₃H₂O), Mobile Phase B: MeCN; Flow rate: 60 mL/min; Gradient: 25% B to 41% B in 8 min; Wavelength: 254 nm/220 nm; RT1 (min): 8.77) to afford [(15R*,18S)-13,20-difluoro-3,21-dimethyl-16-oxo-15-{7-oxofuro[2,3-c]pyridin-6-yl}-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetic acid (17.7 mg, 53.0%) as a white solid. LC-MS: (ES+H, m/z): [M+H]⁺=585.20. ¹H NMR (400 MHz, DMSO-d₆) δ 9.63 (d, J=6.9 Hz, 1H), 8.15 (d, J=2.0 Hz, 1H), 7.48 (ddd, J=7.7, 5.0, 2.1 Hz, 1H), 7.28 (d, J=8.4 Hz, 1H), 7.21-7.10 (m, 4H), 6.93 (dd, J=9.5, 7.0 Hz, 2H), 6.86 (d, J=2.0 Hz, 1H), 6.81 (d, J=7.5 Hz, 1H), 6.45 (dd, J=7.2, 3.3 Hz, 2H), 5.50 (q, J=6.7 Hz, 1H), 5.36 (d, J=13.1 Hz, 1H), 4.98 (d, J=13.1 Hz, 1H), 2.45-2.32 (m, 2H), 2.30-2.21 (m, 3H), 1.88 (s, 3H). ¹⁹F NMR (377 MHz, DMSO-d₆) δ −117.257, −127.684.

### Example 101

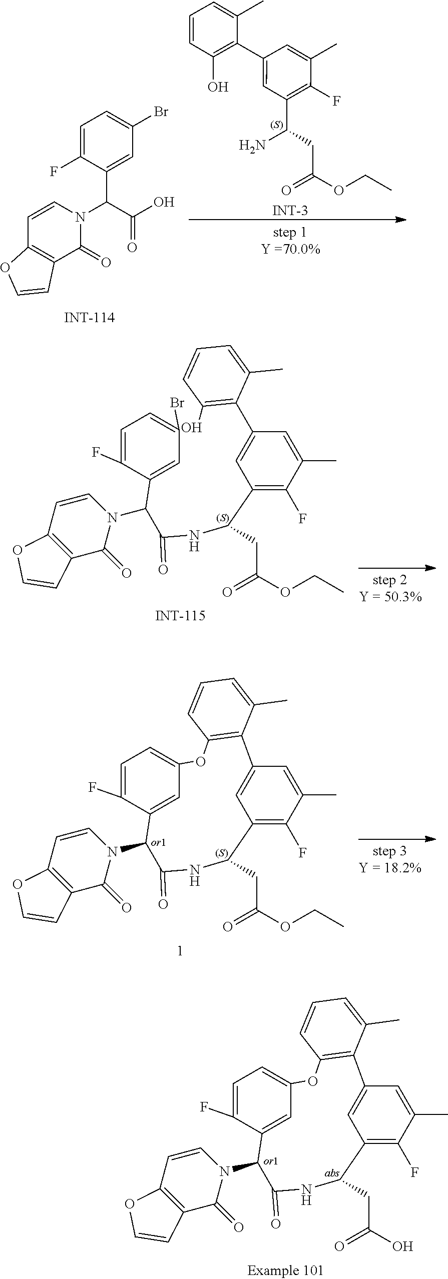

### Step 1: Preparation of ethyl (3S)-3-(2-(5-bromo-2-fluorophenyl)-2-(4-oxofuro[3,2-c]pyridin-5(4H)-yl)acetamido)-3-(4-fluoro-2′-hydroxy-5,6′-dimethyl-[1,1′-biphenyl]-3-yl)propanoate

To a stirred mixture of 2-(5-bromo-2-fluorophenyl)-2-(4-oxofuro[3,2-c]pyridin-5(4H)-yl)acetic acid (200 mg, 0.55 mmol, 1.00 equiv.) in MeCN (5 mL) was added HOBT (149 mg, 1.10 mmol, 2.00 equiv.), EDCI (211 mg, 1.10 mmol, 2.00 equiv.) at room temperature under nitrogen atmosphere. The resulting mixture was stirred for 1 h at room temperature under nitrogen atmosphere. To the above mixture was added ethyl (3S)-3-amino-3-{4-fluoro-2′-hydroxy-5,6′-dimethyl-[1,1′-biphenyl]-3-yl}propanoate (165 mg, 0.55 mmol, 1.00 equiv.) and TEA (332 mg, 3.30 mmol, 6.00 equiv.) in MeCN (5 mL) in portions over 1 min at room temperature. The resulting mixture was stirred for additional overnight at 40° C. under nitrogen atmosphere. The resulting mixture was concentrated under reduced pressure. The residue was dissolved in MeCN (10 mL). The residue was purified by reversed-phase flash chromatography (C18 gel; mobile phase, MeCN in water (0.1% HCOOH), 60% to 100% gradient in 20 min; detector, UV 254 nm) to afford ethyl (3S)-3-[2-(5-bromo-2-fluorophenyl)-2-{4-oxofuro[3,2-c]pyridin-5-yl}acetamido]-3-{4-fluoro-2′-hydroxy-5,6′-dimethyl-[1,1′-biphenyl]-3-yl}propanoate (280 mg, 70.1%, mixture) as a yellow solid. LC-MS: (ES+H, m/z): [M+H]⁺=679.00/681.10. ¹H NMR (300 MHz, DMSO-d₆) δ 9.44-9.28 (m, 1H), 9.10 (d, J=5.1 Hz, 1H), 8.00-7.84 (m, 1H), 7.66 (d, J=22.6 Hz, 1H), 7.51-6.89 (m, 8H), 6.85-6.59 (m, 3H), 5.64 (s, 1H), 4.14-4.01 (m, 2H), 2.92-2.64 (m, 2H), 2.26 (d, J=9.1 Hz, 3H), 1.90 (s, 3H), 1.25-1.12 (m, 3H).

### Step 2: Preparation of ethyl 2-[(14S,17S)-12,19-difluoro-3,20-dimethyl-15-oxo-14-{4-oxofuro[3,2-c]pyridin-5-yl}-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (Intermediate 115)

To a stirred mixture of ethyl (3S)-3-[2-(5-bromo-2-fluorophenyl)-2-{4-oxofuro[3,2-c]pyridin-5-yl}acetamido]-3-{4-fluoro-2′-hydroxy-5,6′-dimethyl-[1,1′-biphenyl]-3-yl}propanoate (100 mg, 0.15 mmol, 1.00 equiv.) and (1S,2S)—N1,N2-dimethylcyclohexane-1,2-diamine (21 mg, 0.15 mmol, 1.00 equiv.) in dioxane (5 mL) were added CuI (14 mg, 0.07 mmol, 0.50 equiv.) and K₂CO₃ (61 mg, 0.44 mmol, 3.00 equiv.). The resulting mixture was stirred for 4 h at 100° C. under nitrogen atmosphere. The resulting mixture was diluted with water (20 mL). The resulting mixture was extracted with EtOAc (3×20 mL). The combined organic layers dried over anhydrous Na₂SO₄, filtered, and concentrated. The crude product (100 mg) was purified by Prep-HPLC (Column: XBridge Shield RP18 OBD Column, 30*150 mm, 5 μm; Mobile Phase A: Water (10 mmol/L NH₄HCO₃), Mobile Phase B: MeCN; Flow rate: 60 mL/min; Gradient: 54% B to 56% B in 10 min; Wavelength: 254 nm/220 nm; RT1 (min): 9.1) to afford ethyl 2-[(14S,17S)-12,19-difluoro-3,20-dimethyl-15-oxo-14-{4-oxofuro[3,2-c]pyridin-5-yl}-8-oxa-16-azatetracyclo [16.3.1.1\hat{ }{9,13}.0\hat{ }{2,7}]tricosa-1(22),2(7),3,5,9,11,13(23),18,20-nonaen-17-yl]acetate (32 mg, 36.3%) as a white solid. LC-MS: (ES+H, m/z): [M+H]⁺=599.20.

### Step 3: Preparation of [(14S,17S)-12,19-difluoro-3,20-dimethyl-15-oxo-14-{4-oxofuro[3,2-c]pyridin-5-yl}-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid

To a stirred solution of ethyl 2-[(14S,17S)-12,19-difluoro-3,20-dimethyl-15-oxo-14-{4-oxofuro[3,2-c]pyridin-5-yl}-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (30 mg, 0.050 mmol, 1.00 equiv.) in THF (5 mL) and H₂O (1 mL) was added LiOH·H₂O (3.60 mg, 0.150 mmol, 3.00 equiv.). The resulting mixture was stirred for 2 h at 25° C. under nitrogen atmosphere. The resulting mixture was concentrated under reduced pressure. The crude product (30 mg) was purified by Prep-HPLC (Column: Xselect CSH C18 OBD Column 30*150 mm, 5 μm; Mobile Phase A: Water (0.1% HCOOH), Mobile Phase B: MeCN; Flow rate: 60 mL/min; Gradient: 48% B to 64% B in 8 min; Wavelength: 254/220 nm; RT1 (min): 8.10) to afford [(14S,17S)-12,19-difluoro-3,20-dimethyl-15-oxo-14-{4-oxofuro[3,2-c]pyridin-5-yl}-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid (5.3 mg, 18.2%) as a white solid. LC-MS: (ES+H, m/z): [M+H]⁺=571.25. ¹H NMR (400 MHz, CD₃OD) δ 7.71 (d, J=2.2 Hz, 1H), 7.51 (ddd, J=9.1, 4.1, 2.9 Hz, 1H), 7.43 (d, J=8.1 Hz, 1H), 7.33 (t, J=7.9 Hz, 1H), 7.18-7.08 (m, 2H), 7.04 (d, J=7.6 Hz, 1H), 7.00-6.94 (m, 3H), 6.62 (dd, J=7.7, 0.9 Hz, 1H), 6.47 (dd, J=6.3, 2.2 Hz, 1H), 6.07 (dd, J=6.2, 2.9 Hz, 1H), 5.71 (t, J=7.6 Hz, 1H), 2.82 (dd, J=15.7, 8.3 Hz, 1H), 2.70 (dd, J=15.7, 7.0 Hz, 1H), 2.34 (d, J=2.0 Hz, 3H), 2.00 (s, 3H). ¹⁹F NMR (377 MHz, CD₃OD) δ −123.49, −126.73.

### Example 102

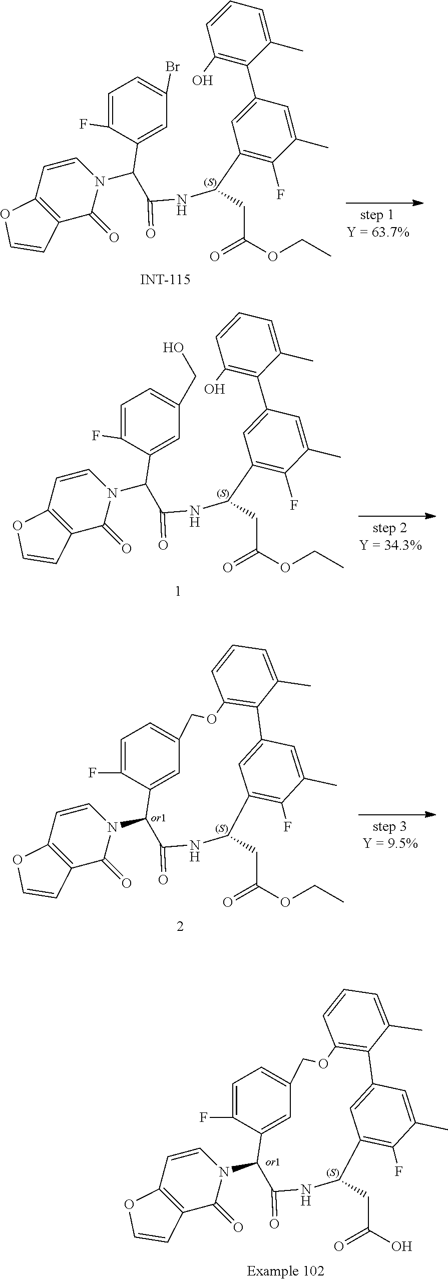

### Step 1: Preparation of ethyl (3S)-3-(4-fluoro-2′-hydroxy-5,6′-dimethyl-[1,1′-biphenyl]-3-yl)-3-(2-(2-fluoro-5-(hydroxymethyl)phenyl)-2-(4-oxofuro[3,2-c]pyridin-5(4H)-yl)acetamido)propanoate

To a stirred mixture of ethyl (3S)-3-[2-(5-bromo-2-fluorophenyl)-2-{4-oxofuro[3,2-c]pyridin-5-yl}acetamido]-3-{4-fluoro-2′-hydroxy-5,6′-dimethyl-[1,1′-biphenyl]-3-yl}propanoate (200 mg, 0.29 mmol, 1.00 equiv.) and (tributylstannyl)methanol (284 mg, 0.88 mmol, 3.00 equiv.) in dioxane (10 mL) was added 2nd generation XPhos precatalyst (69 mg, 0.09 mmol, 0.30 equiv.). The resulting mixture was stirred for 16 h at 80° C. under nitrogen atmosphere. The mixture was allowed to cool down to room temperature. The resulting mixture was concentrated under reduced pressure. The residue was purified by reversed-phase flash chromatography (C18 gel; mobile phase, MeCN in water (0.1% NH₃·H₂O), 20% to 60% gradient in 20 min; detector, UV 254 nm) to afford ethyl (3S)-3-(4-fluoro-2′-hydroxy-5,6′-dimethyl-[1,1′-biphenyl]-3-yl)-3-(2-(2-fluoro-5-(hydroxymethyl)phenyl)-2-(4-oxofuro[3,2-c]pyridin-5(4H)-yl)acetamido)propanoate (130 mg, 63.7%, mixture) as a white solid. LC-MS: (ES+H, m/z): [M+H]⁺=631.25

### Step 2: Preparation of ethyl 2-[(15S,18S)-13,20-difluoro-3,21-dimethyl-16-oxo-15-{4-oxofuro[3,2-c]pyridin-5-yl}-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetate

A mixture of ethyl (3S)-3-{4-fluoro-2′-hydroxy-5,6′-dimethyl-[1,1′-biphenyl]-3-yl}-3-{2-[2-fluoro-5-(hydroxymethyl)phenyl]-2-{4-oxofuro[3,2-c]pyridin-5-yl}acetamido}propanoate (160 mg, 0.25 mmol, 1.00 equiv.) and 2-(tributylphosphanylidene)acetonitrile (296 mg, 1.23 mmol, 5.00 equiv.) in toluene (20 mL) was stirred overnight at 100° C. under nitrogen atmosphere. The mixture was allowed to cool down to room temperature. The resulting mixture was concentrated under reduced pressure. The residue was purified by reversed-phase flash chromatography (C18 gel; mobile phase, MeCN in water (10 mmol/L NH₄HCO₃), 30% to 70% gradient in 30 min; detector, UV 254 nm) to provide ethyl 2-[(15S,18S)-13,20-difluoro-3,21-dimethyl-16-oxo-15-{4-oxofuro[3,2-c]pyridin-5-yl}-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetate (55 mg, 34.3%) as a light brown oil. LC-MS: (ES+H, m/z): [M+H]⁺=613.2

### Step 3: Preparation of [(15R*,18S)-13,20-difluoro-3,21-dimethyl-16-oxo-15-{4-oxofuro[3,2-c]pyridin-5-yl}-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetic acid

A mixture of ethyl 2-[(15S,18S)-13,20-difluoro-3,21-dimethyl-16-oxo-15-{4-oxofuro[3,2-c]pyridin-5-yl}-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetate (50 mg, 0.082 mmol, 1.00 equiv.) and LiOH·H₂O (13.70 mg, 0.327 mmol, 4.00 equiv.) in THF (4 mL) and H₂O (1 mL) was stirred for 4 h at 30° C. under nitrogen atmosphere. The resulting mixture was concentrated under reduced pressure. The crude product (50 mg) was purified by Prep-HPLC (Column: XBridge Shield RP18 OBD Column, 30*150 mm, 5 μm; Mobile Phase A: Water (10 mmol/L NH₄HCO₃+0.05% NH₃·H₂O), Mobile Phase B: MeCN; Flow rate: 60 mL/min; Gradient: 16% B to 36% B in 10 min; Wavelength: 254 nm/220 nm; RT1 (min): 9.1) to afford [(15R*,18S)-13,20-difluoro-3,21-dimethyl-16-oxo-15-{4-oxofuro[3,2-c]pyridin-5-yl}-8-oxa-17-azatetracyclo[17.3.1.1\hat{ }{10,14}.0\hat{ }{2,7}]tetracosa-1(23),2(7),3,5,10,12,14(24),19,21-nonaen-18-yl]acetic acid (4.7 mg, 9.5%) as a white solid. LC-MS: (ES−H, m/z): [M+H]⁺=585.20. ¹H NMR (400 MHz, CD₃OD) δ 7.69 (d, J=2.2 Hz, 1H), 7.43 (ddd, J=7.9, 4.9, 2.1 Hz, 1H), 7.33 (dd, J=7.3, 2.2 Hz, 1H), 7.19 (d, J=5.3 Hz, 1H), 7.17-6.95 (m, 5H), 6.92 (dd, J=7.0, 2.2 Hz, 1H), 6.81 (d, J=7.2 Hz, 1H), 6.68 (dd, J=6.7, 2.2 Hz, 1H), 6.57 (d, J=7.6 Hz, 1H), 5.73 (dd, J=8.1, 5.4 Hz, 1H), 5.29 (d, J=13.2 Hz, 1H), 4.97 (d, J=13.1 Hz, 1H), 2.68 (qd, J=15.5, 6.8 Hz, 2H), 2.31 (d, J=2.0 Hz, 3H), 1.97 (s, 3H). ¹⁹F NMR (376 MHz, CD₃OD) δ −118.444, 118.778, −128.370.

### Example 103

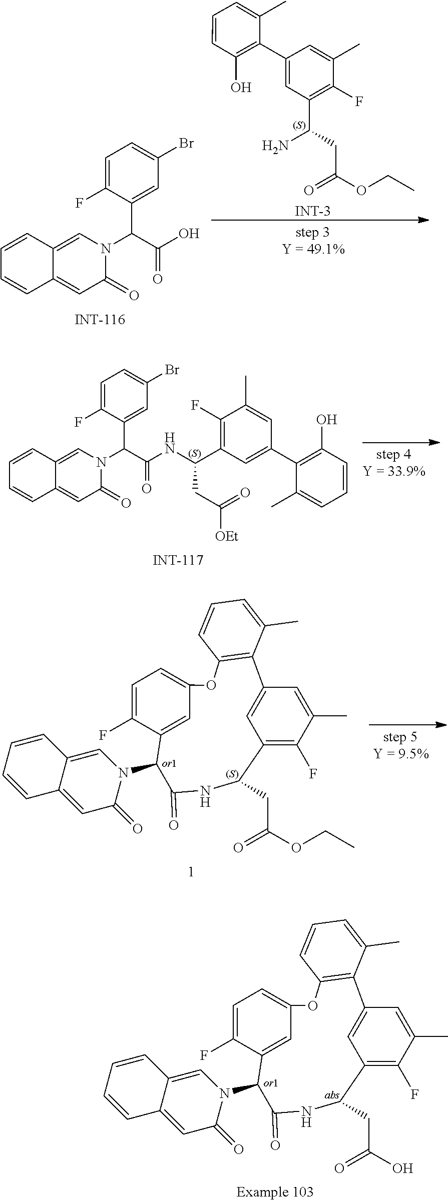

### Step 1: Preparation of ethyl (3S)-3-(2-(5-bromo-2-fluorophenyl)-2-(3-oxoisoquinolin-2(3H)-yl)acetamido)-3-(4-fluoro-2′-hydroxy-5,6′-dimethyl-[1,1′-biphenyl]-3-yl)propanoate

To a stirred solution of (5-bromo-2-fluorophenyl)(3-oxoisoquinolin-2-yl)acetic acid (500 mg, 1.33 mmol, 1.00 equiv.) and TCFH (2.23 g, 7.97 mmol, 6.00 equiv.) in MeCN (20 mL) were added NMI (1.31 g, 15.95 mmol, 12.00 equiv.) and ethyl (3S)-3-amino-3-{4-fluoro-2′-hydroxy-5,6′-dimethyl-[1,1′-biphenyl]-3-yl}propanoate hydrochloride (977 mg, 2.66 mmol, 2.00 equiv.) at 25° C. under nitrogen atmosphere. The resulting mixture was stirred for 16 h at 50° C. and then cooled to room temperature and concentrated. The crude product was purified by reverse phase flash chromatography (C18 gel; mobile phase, MeCN in water (0.1% NH₃·H₂O), 40% to 100% gradient in 30 min; detector, UV 220 nm) to afford ethyl (3S)-3-[2-(5-bromo-2-fluorophenyl)-2-(3-oxoisoquinolin-2-yl)acetamido]-3-{4-fluoro-2′-hydroxy-5,6′-dimethyl-[1,1′-biphenyl]-3-yl}propanoate (600 mg, 49.1%) as a yellow solid. LC-MS: (ES+H, m/z): [M+H]⁺=688.95.

### Step 2: Preparation of ethyl 2-[(14S,17S)-12,19-difluoro-3,20-dimethyl-15-oxo-14-(3-oxoisoquinolin-2-yl)-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate

To a stirred mixture of ethyl (3S)-3-[2-(5-bromo-2-fluorophenyl)-2-(3-oxoisoquinolin-2-yl)acetamido]-3-{4-fluoro-2′-hydroxy-5,6′-dimethyl-[1,1′-biphenyl]-3-yl}propanoate (200 mg, 0.29 mmol, 1.00 equiv.) in dioxane (5 mL) was added CuI (28 mg, 0.14 mmol, 0.50 equiv.), (1R,2R)-1-N,2-N-dimethylcyclohexane-1,2-diamine (41 mg, 0.29 mmol, 1.00 equiv.) and K₂CO₃ (80 mg, 0.58 mmol, 2.00 equiv.) at room temperature. The resulting mixture was stirred for 2 h at 100° C. under nitrogen atmosphere and then diluted with water (10 mL) and extracted with EtOAc (3×10 mL). The combined organic layers were washed with brine (3×10 mL), dried over anhydrous Na₂SO₄, filtered, and concentrated. The residue was purified by silica gel column chromatography (PE/EtOAc (0 to 60% gradient in 50 min) to afford ethyl 2-[(14S,17S)-12,19-difluoro-3,20-dimethyl-15-oxo-14-(3-oxoisoquinolin-2-yl)-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (60 mg, 33.9%) as a yellow solid. LC-MS: (ES+H, m/z): [M+H]⁺=609.2.

### Step 3: Preparation of [(14R*,17S)-12,19-difluoro-3,20-dimethyl-15-oxo-14-(3-oxoisoquinolin-2-yl)-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid

To a stirred mixture of ethyl 2-[(14R*,17S)-12,19-difluoro-3,20-dimethyl-15-oxo-14-(3-oxoisoquinolin-2-yl)-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetate (60 mg, 0.09 mmol, 1.00 equiv.) in THF (1 mL) and H₂O (0.2 mL) was added LiOH·H₂O (12 mg, 0.50 mmol, 5.00 equiv.) in portions at room temperature. The resulting mixture was stirred for 5 h at 30° C. under nitrogen atmosphere and then cooled to room temperature and concentrated. The crude product was purified by Prep-HPLC (Column: Xselect CSH Fluoro-Phenyl Column, 30*150 mm, 5 μm; Mobile Phase A: Water (0.1% HCOOH), Mobile Phase B: MeCN; Flow rate: 60 mL/min; Gradient: 32% B to 52% B in 10 min; Wavelength: 254 nm/220 nm; RT1 (min): 8.17) to afford [(14R*,17S)-12,19-difluoro-3,20-dimethyl-15-oxo-14-(3-oxoisoquinolin-2-yl)-8-oxa-16-azatetracyclo[16.3.1.1\hat{ }{9,13}.0\hat{ }{2,7}]tricosa-1(22),2(7),3,5,9,11,13(23),18,20-nonaen-17-yl]acetic acid (5.5 mg, 9.5%) as a light yellow solid. LC-MS: (ES+H, m/z): [M+H]⁺=581.15. ¹H NMR (400 MHz, DMSO-d₆) δ 12.50 (s, 1H), 9.93 (s, 1H), 7.63 (d, J=16.1 Hz, 1H), 7.55-7.44 (m, 2H), 7.40-7.26 (m, 4H), 7.25-7.13 (m, 3H), 7.03 (s, 1H), 6.90-6.76 (m, 2H), 6.50 (s, 1H), 6.34 (s, 1H), 5.23 (s, 1H), 3.83 (d, J=17.3 Hz, 1H), 3.43 (d, J=17.4 Hz, 1H), 2.24 (d, J=2.0 Hz, 3H), 2.11 (s, 3H). ¹⁹F NMR (376 MHz, DMSO-d₆) δ −122.47, −125.29.

a4b7 Cell Capture Assay

The potency of inhibitors in preventing a4b7 integrin interaction with MadCAM-1 was measured by monitoring the capture of a4b7 integrin expressing cells, RPMI8866, on recombinant human MAdCAM-1 Fc chimera protein-coated plates. The protein was a fusion between human MAdCAM-1 (Met1-Gln333, Accession #AAY82472) and human IgG₁ (Pro100-Lys330)] (R&D Systems, Catalog #6056-MC). 96-well plates (Thermofisher, Catalog #136102) were coated with the recombinant protein by dispensing 100 μL (1.0 μg/mL) per well. After a quick spin to settle down the coating solution using a plate centrifuge, plates were incubated overnight at 4° C. in a moisture chamber. On Day 2, the plates were washed with PBS using a Bluecatbio Blue®Washer (GentleSpin wash, 3 cycles, pressure=1, volume 150 uL). Blocking solution (1.0% BSA in PBS, Sigma, Catalog #A7979) was then added at 200 uL/well, and the plates were quickly spun to settle down blocking buffer and incubated in cell culture incubator at 37° C. and 5% CO₂ for 1 hour. The plates were then washed with 0.1% BSA in PBS using a Bluecatbio Blue®Washer (GentleSpin wash, 3 cycles, pressure=1, volume 150 uL). RPMI8866 cells suspended in assay medium (1 mM MgCl₂ 25 mM HEPES and 50% human serum, Sigma, Catalog #H4522) at a density of 1.0×10\hat{ }6 cells/mL were added at 100 uL/well. Each compound evaluated was prepared as a DMSO stock solution. Test compounds were evaluated with a 9-point serial dilution curve (1000, 333.3, 111.1, 55.6, 27.8, 13.9, 6.9, 2.3, and 0.77 nM)) and were added to the plates using a Tecan D.30e Digita dispenser, the dispensed stock is 100 nL and the final DMSO concentration is 0.1%. Potent compounds were re-evaluated at a new 9-point serial dilution curve (100, 33.3, 11.1, 5.6, 2.8, 1.4, 0.69, 0.23, and 0.08 nM). Each titration curves were performed in triplicate on the same plate, and most of compounds were retested in different dates multiple times. The plates were shaken at 300 rpm on orbital plate shaker for 3 minutes to mix the cells and compounds, quickly spun to settle the cells and solution, and incubated in cell culture incubator at 37° C. and 5% CO₂. After 1 hour incubation, the unbound cells were washed away with RPMI1640 medium supplemented with 1 mM MgCl₂ and 1% BSA) (wash buffer) using a Bluecatbio Blue®Washer (MagbeadsSpin wash, 1 cycle, pressure 1, volume=150 uL). After wash buffer removal, A mixture of wash buffer/CellTiter-Glo® Reagent (Pormega, catalog #G7573) at 1:1 (V/V) was added at volume of 100 uL to each well. The plates were placed in a chamber at 25° C. and incubated for 30 minutes. Luminescence was recorded using a Envision plate reader 2105 according to CellTiter-Glo protocol.

The mean luminescence signals from negative control wells without MadCAM-1 coating and compound treatment (cells only) was defined as 100% binding inhibition, and the mean luminescence signals from positive control wells with MadCAM-1 coating but without compound treatment (MadCAM-1 and cells only) was defined as 0% binding inhibition. The luminescence signals from other compound treated wells were normalized in percentage scale accordingly. The normalized mean % inhibition data were plotted against logarithmic compound concentration in nanomolar and were fitted with 4-parameter logistic model to obtain IC50 for each compound.

a4b1 Cell Capture Assay

The potency of inhibitors in preventing a4b1 integrin interaction with VCAM-1 was measured by monitoring the capture of a4b1 integrin expressing cells Jurkat on recombinant human VCAM-1 Fc chimera protein-coated plates. The protein was a fusion between human VCAM-1 (Phe25-Glu698) Accession #P19320, Accession #AAY82472) and human IgG₁ (Pro100-Lys330)] (R&D Systems, Catalog #862-VC). 96-well plates (Thermofisher, Catalog #136102) were coated with the recombinant protein by dispensing 100 μL of 0.25 μg/mL per well. After a quick spin to settle down the coating solution using plate centrifuge, plates were incubated overnight at 4° C. in a moisture chamber. On Day 2, the plates were washed with PBS using a Bluecatbio Blue®Washer (GentleSpin wash, 3 cycles, pressure=1, volume 150 uL). Blocking solution (1.0% BSA in PBS, Sigma, Catalog #A7979) was then added at 200 uL/well, and the plates were quickly spun to settle down blocking buffer and incubated in cell culture incubator at 37° C. and 5% CO₂ for 1 hour. The plates were then washed with 0.1% BSA in PBS using a Bluecatbio Blue®Washer (GentleSpin wash, 3 cycles, pressure=1, volume 150 uL). Jurkat cells suspended in assay medium (1 mM MgCl2 25 mM HEPES and 50% human serum, Sigma, Catalog #H4522) at a density of 0.5×10 6 cells/mL were added at 100 uL/well. Each compound evaluated was prepared as a DMSO stock solution. Test compounds were evaluated with a 9-point serial dilution curve and were added to the plates using a Tecan D300e Digital dispenser. Each titration curves were performed in triplicate on the same plate, and most of compounds were retested in different dates multiple times. The plates were shaken at 300 rpm on orbital plate shaker for 3 minutes to mix the cells and compounds, quickly spun to settle the cells and solution, and incubated in cell culture incubator at 37° C. and 5% CO₂. After 1 hour incubation, the unbound cells were washed away with RPMI1640 medium supplemented with 1 mM MgCl₂ and 1% BSA) (wash buffer) using a Bluecatbio Blue®Washer (MagbeadsSpin wash, 1 cycle, pressure 1, volume=150 uL). After wash buffer removal, A mixture of wash buffer/CellTiter-Glo® Reagent (Pormega, catalog #G7573) at 1:1 (V/V) was added at volume of 100 uL to each well. The plates were placed in a chamber at 25° C. and incubated for 30 minutes. Luminescence was recorded using a Envision plate reader 2105 according to CellTiter-Glo protocol.

The mean luminescence signals from negative control wells without VCAM-1 coating and compound treatment (cells only) was defined as 100% binding inhibition, and the mean luminescence signals from positive control wells with VCAM-1 coating but without compound treatment (VCAM-1 and cells only) was defined as 0% binding inhibition. The luminescence signals from other compound treated wells were normalized in percentage scale accordingly. The normalized mean % inhibition data were plotted against logarithmic compound concentration in nanomolar and were fitted with 4-parameter logistic model to obtain IC50 for each compound.

The assay data for the compounds disclosed herein are provided in Table 3.

TABLE 3

| | a4B7 cell capture | a4B1 cell capture |
|---|---|---|
| | assay RMPI8866- | assay Jurkat- |
| Ex. | MadCAM EC₅₀ | VCAM-1 EC₅₀ |
| No. | (M) | (M) |
| 1 | 5.90E−08 | 2.08E−06 |
| 2 | 7.60E−08 | |
| 3 | 1.20E−08 | 3.59E−07 |
| 4 | 4.40E−07 | |
| 5 | 3.30E−07 | |
| 6 | 2.30E−07 | |
| 7 | 1.10E−09 | 6.15E−08 |
| 8 | 1.00E−07 | |
| 9 | 7.20E−08 | |
| 10 | 3.60E−07 | 1.19E−05 |
| 11 | 3.40E−07 | 4.58E−06 |
| 12 | 9.70E−07 | |
| 13 | >1.0E−06 | |
| 14 | 3.80E−08 | |
| 15 | 2.80E−08 | 8.80E−07 |
| 16 | 1.40E−08 | |
| 17 | 1.40E−07 | |
| 18 | 9.20E−09 | 6.89E−07 |
| 19 | 8.40E−10 | |
| 20 | 8.00E−11 | |
| 21 | 7.90E−11 | |
| 22 | 9.90E−09 | |
| 23 | 2.30E−09 | |
| 24 | 1.90E−09 | |
| 25 | 2.00E−09 | |
| 26 | 1.30E−09 | |
| 27 | 3.70E−09 | |
| 28 | 1.90E−08 | |
| 29 | 7.00E−11 | |
| 30 | 2.60E−09 | |
| 31 | 1.90E−09 | |
| 32 | 9.50E−10 | |
| 33 | 5.00E−08 | |
| 34 | 2.30E−08 | |
| 35 | 7.70E−09 | |
| 36 | 5.20E−09 | |
| 37 | 1.30E−09 | |
| 38 | 1.30E−09 | |
| 39 | >1.0E−07 | |
| 40 | 4.20E−08 | |
| 41 | 2.10E−08 | |
| 42 | 1.30E−08 | |
| 43 | 4.80E−07 | |
| 44 | 1.60E−07 | |
| 45 | 1.80E−09 | |
| 46 | 1.60E−09 | |
| 47 | 4.20E−08 | |
| 48 | 5.00E−10 | |
| 49 | 4.90E−09 | |
| 50 | >1.0E−07 | |
| 51 | 9.90E−07 | |
| 52 | 1.80E−07 | |
| 53 | 1.10E−08 | |
| 54 | 2.70E−08 | |
| 55 | 4.00E−09 | |
| 56 | 9.70E−10 | |
| 57 | 2.60E−09 | |
| 58 | 7.00E−09 | |
| 59 | 1.20E−09 | |
| 60 | 3.80E−07 | |
| 61 | 4.20E−08 | |
| 62 | 3.30E−08 | |
| 63 | 1.00E−08 | |
| 64 | 6.40E−09 | |
| 65 | 7.70E−08 | |
| 66 | 4.00E−10 | |
| 67 | 1.90E−07 | |
| 69 | 3.80E−10 | |
| 70 | >1.0E−07 | |
| 71 | 3.50E−09 | |
| 72 | 1.60E−08 | |
| 73 | 2.70E−08 | |
| 74 | 2.00E−08 | |
| 75 | >1.0E−07 | |
| 76 | 3.90E−08 | |
| 77 | 1.50E−08 | |
| 78 | 3.60E−07 | |
| 79 | >1.0E−07 | |
| 80 | 5.80E−08 | |

1. A compound of Formula (I), or a pharmaceutically acceptable salt, solvate, or stereoisomer thereof: 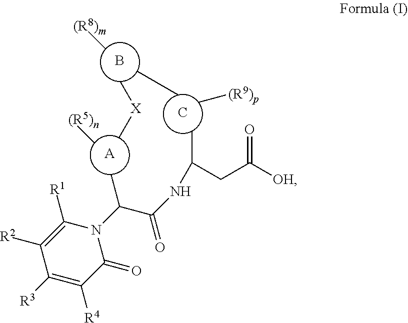
wherein:
R¹ is hydrogen, deuterium, halogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl; wherein the alkyl, cycloalkyl, and heterocycloalkyl is optionally substituted with one or more R;
R² is hydrogen, deuterium, halogen, -L-CN, -L-OH, -L-OR^{a}, -L-S(═O)R^{a}, -L-S(═O)₂R^{a}, -L-S(═O)₂NR^{c}R^{d}, -L-NR^{c}R^{d}, -L-C(═O)R^{a}, -L-C(═O)OR^{b}, -L-C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, -L-cycloalkyl, or -L-heterocycloalkyl; wherein the alkyl, alkenyl, alkynyl, cycloalkyl, and heterocycloalkyl is optionally substituted with one or more R;
R³ is hydrogen, deuterium, halogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl; wherein the alkyl, cycloalkyl, and heterocycloalkyl is optionally substituted with one or more R;
R⁴ is hydrogen, deuterium, halogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl; wherein the alkyl, cycloalkyl, and heterocycloalkyl is optionally substituted with one or more R;
L is absent or C₁-C₆alkylene;
Ring A is aryl or heteroaryl;
each R⁵ is independently halogen, —CN, —NO₂, —OH, —OR^{a}, —SH, —SR^{a}, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or two R⁵ are taken together to form an oxo;
n is 0-4;
X is —O—, —S—, —C(R⁶)₂—, —NR⁷—, —[C(R⁶)₂]₂—, —O—C(R⁶)₂—, —C(R⁶)₂—O—, —S—C(R⁶)₂—, or —C(R⁶)₂—S—;
each R⁶ is independently hydrogen, deuterium, halogen, —OH, —OR^{a}, C₁-C₆alkyl, C₁-C₆haloalkyl, C₁-C₆hydroxyalkyl, C₁-C₆aminoalkyl, or C₁-C₆heteroalkyl;
or two R⁶ are taken together to form a cycloalkyl or heterocycloalkyl; each optionally substituted with one or more R;
R⁷ is hydrogen, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl;
Ring B is aryl or heteroaryl;
each R¹ is independently halogen, —CN, —NO₂, —OH, —OR^{a}, —SH, —SR^{a}, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or two R⁸ are taken together to form an oxo;
m is 0-4;
Ring C is aryl or heteroaryl;
each R⁹ is independently halogen, —CN, —NO₂, —OH, —OR^{a}, —SH, —SR^{a}, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or two R⁹ are taken together to form an oxo;
p is 0-4;
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each alkyl, alkylene, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each alkyl, alkylene, alkenyl, alkynyl, cycloalkyl, heterocycloalkyl, aryl, and heteroaryl is independently optionally substituted with one or more R;
R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each alkyl, alkylene, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R; and
each R is independently halogen, —CN, —OH, —OC₁-C₃alkyl, —OC₁-C₃haloalkyl, —SC₁-C₃alkyl, —S(═O)C₁-C₃alkyl, —S(═O)₂C₁-C₃alkyl, —S(═O)₂NH₂, —S(═O)₂NHC₁-C₃alkyl, —S(═O)₂N(C₁-C₃alkyl)₂, —NH₂, —NHC₁-C₃alkyl, —N(C₁-C₃alkyl)₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, C₁-C₃alkyl, C₁-C₃haloalkyl, C₁-C₃deuteroalkyl, C₁-C₃hydroxyalkyl, C₁-C₃aminoalkyl, C₁-C₃heteroalkyl, C₃-C₆cycloalkyl, or heterocycloalkyl;
or two R on the same atom are taken together to form an oxo.

2-6. (canceled)

7. The compound of claim 1, or a pharmaceutically acceptable salt, solvate, or stereoisomer thereof, wherein Ring A is phenyl.

8-11. (canceled)

12. The compound of claim 1, or a pharmaceutically acceptable salt, solvate, or stereoisomer thereof, wherein each R⁵ is independently halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl; wherein each alkyl, cycloalkyl, and heterocycloalkyl is independently optionally substituted with one or more R.

13-17. (canceled)

18. The compound of claim 1, or a pharmaceutically acceptable salt, solvate, or stereoisomer thereof, wherein X is —O—, —S—, —C(R⁶)₂—, or —NR⁷—.

19. The compound of claim 1, or a pharmaceutically acceptable salt, solvate, or stereoisomer thereof, wherein X is —O—.

20-24. (canceled)

25. The compound of claim 1, or a pharmaceutically acceptable salt, solvate, or stereoisomer thereof, wherein each R⁶ is hydrogen.

26-29. (canceled)

30. The compound of claim 1, or a pharmaceutically acceptable salt, solvate, or stereoisomer thereof, wherein Ring B is phenyl.

31-32. (canceled)

33. The compound of claim 1, or a pharmaceutically acceptable salt, solvate, or stereoisomer thereof, wherein Ring B is 5-membered heteroaryl.

34. (canceled)

35. The compound of claim 1, or a pharmaceutically acceptable salt, solvate, or stereoisomer thereof, wherein each R¹ is independently halogen, —CN, —NO₂, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl; wherein each alkyl, cycloalkyl, and heterocycloalkyl is independently optionally substituted with one or more R.

36-40. (canceled)

41. The compound of claim 1, or a pharmaceutically acceptable salt, solvate, or stereoisomer thereof, wherein Ring C is phenyl.

42-43. (canceled)

44. The compound of claim 1, or a pharmaceutically acceptable salt, solvate, or stereoisomer thereof, wherein Ring C is 5-membered heteroaryl.

45. The compound claim 1, or a pharmaceutically acceptable salt, solvate, or stereoisomer thereof, wherein each R⁹ is independently halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl; wherein each alkyl, cycloalkyl, and heterocycloalkyl is independently optionally substituted with one or more R.

46. (canceled)

47. The compound of claim 1, or a pharmaceutically acceptable salt, solvate, or stereoisomer thereof, wherein each R⁹ is independently halogen, C₁-C₆alkyl, or C₁-C₆haloalkyl.

48-50. (canceled)

51. The compound of claim 1, or a pharmaceutically acceptable salt, solvate, or stereoisomer thereof, wherein R¹ is hydrogen, deuterium, halogen, C₁-C₆alkyl, or C₁-C₆haloalkyl; wherein the alkyl is optionally substituted with one or more R.

52-54. (canceled)

55. The compound of claim 1, or a pharmaceutically acceptable salt, solvate, or stereoisomer thereof, wherein R³ is hydrogen, deuterium, halogen, C₁-C₆alkyl, or C₁-C₆haloalkyl; wherein the alkyl is optionally substituted with one or more R.

56-57. (canceled)

58. The compound of claim 1, or a pharmaceutically acceptable salt, solvate, or stereoisomer thereof, wherein R⁴ is hydrogen, deuterium, halogen, C₁-C₆alkyl, or C₁-C₆haloalkyl; wherein the alkyl is optionally substituted with one or more R.

59-60. (canceled)

61. The compound of claim 1, or a pharmaceutically acceptable salt, solvate, or stereoisomer thereof, wherein R² is -L-CN, -L-OH, -L-OR^{a}, -L-NR^{c}R^{d}, C₁-C₆alkyl, C₁-C₆haloalkyl, C₁-C₆hydroxyalkyl, C₁-C₆aminoalkyl, C₁-C₆heteroalkyl, -L-cycloalkyl, or -L-heterocycloalkyl; wherein the alkyl, cycloalkyl, and heterocycloalkyl is optionally substituted with one or more R.

62-67. (canceled)

68. A compound, or a pharmaceutically acceptable salt, solvate, or stereoisomer thereof, selected from:

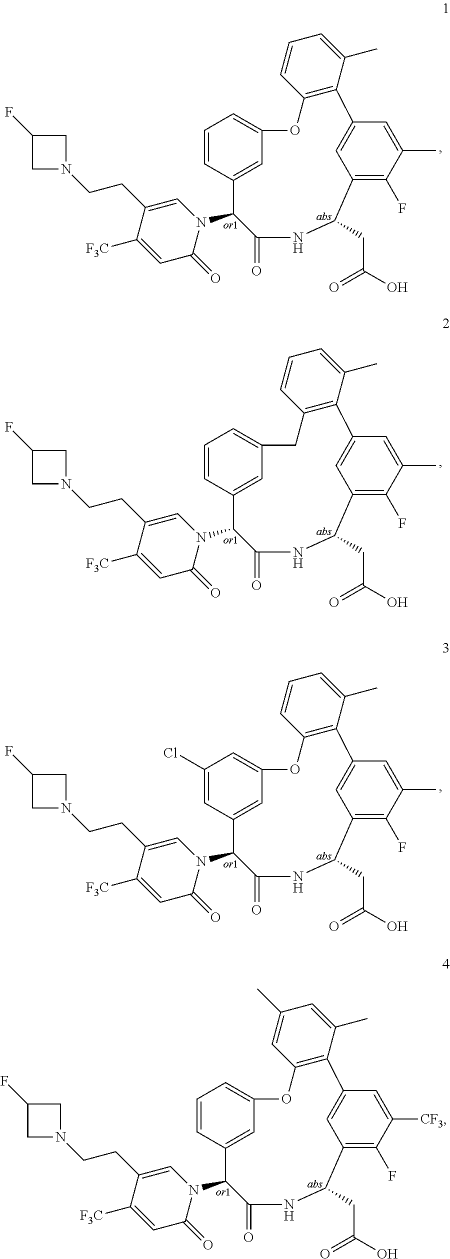 [SMILES: Cc1cc2cc(c1F)[C@H](CC(=O)O)NC(=O)[C@@H](n1cc(CCN3CC(F)C3)c(C(F)(F)F)cc1=O)c1cccc(c1)Oc1cccc(C)c1-2 ; Cc1cc2cc(c1F)[C@H](CC(=O)O)NC(=O)[C@H](n1cc(CCN3CC(F)C3)c(C(F)(F)F)cc1=O)c1cccc(c1)Cc1cccc(C)c1-2 ; Cc1cc2cc(c1F)[C@H](CC(=O)O)NC(=O)[C@@H](n1cc(CCN3CC(F)C3)c(C(F)(F)F)cc1=O)c1cc(Cl)cc(c1)Oc1cccc(C)c1-2 ; Cc1cc(C)c2c(c1)Oc1cccc(c1)[C@H](n1cc(CCN3CC(F)C3)c(C(F)(F)F)cc1=O)C(=O)N[C@@H](CC(=O)O)c1cc-2cc(C(F)(F)F)c1F]

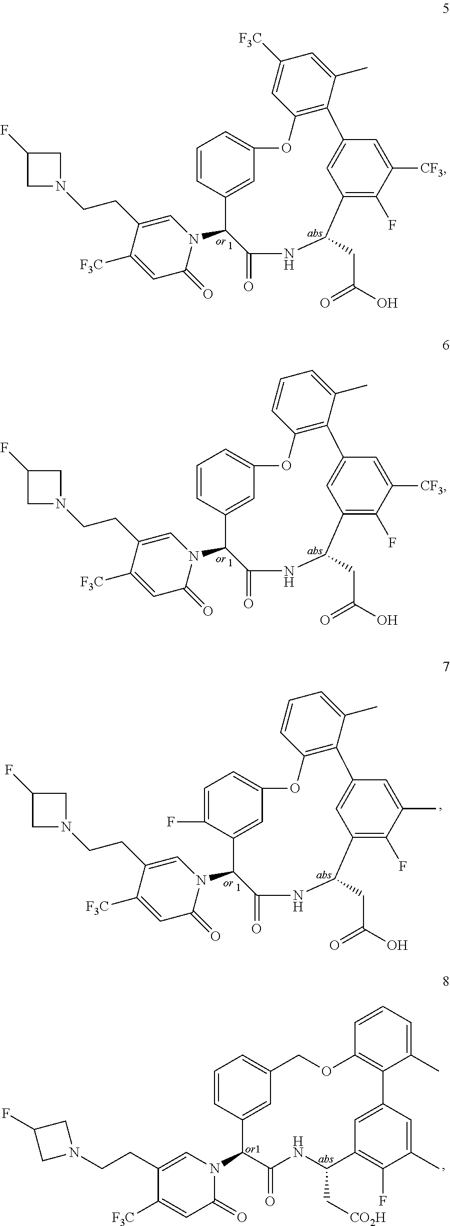 [SMILES: Cc1cc(C(F)(F)F)cc2c1-c1cc(c(F)c(C(F)(F)F)c1)[C@H](CC(=O)O)NC(=O)[C@@H](n1cc(CCN3CC(F)C3)c(C(F)(F)F)cc1=O)c1cccc(c1)O2 ; Cc1cccc2c1-c1cc(c(F)c(C(F)(F)F)c1)[C@H](CC(=O)O)NC(=O)[C@@H](n1cc(CCN3CC(F)C3)c(C(F)(F)F)cc1=O)c1cccc(c1)O2 ; Cc1cc2cc(c1F)[C@H](CC(=O)O)NC(=O)[C@@H](n1cc(CCN3CC(F)C3)c(C(F)(F)F)cc1=O)c1cc(ccc1F)Oc1cccc(C)c1-2 ; Cc1cc2cc(c1F)[C@H](CC(=O)O)NC(=O)[C@@H](n1cc(CCN3CC(F)C3)c(C(F)(F)F)cc1=O)c1cccc(c1)COc1cccc(C)c1-2]

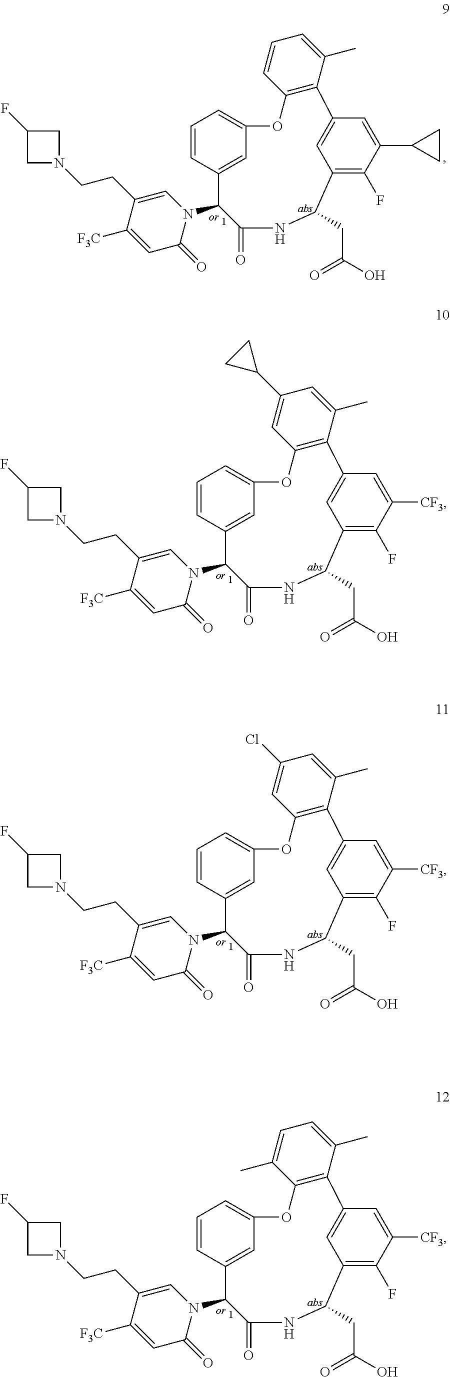 [SMILES: Cc1cccc2c1-c1cc(C3CC3)c(F)c(c1)[C@H](CC(=O)O)NC(=O)[C@@H](n1cc(CCN3CC(F)C3)c(C(F)(F)F)cc1=O)c1cccc(c1)O2 ; Cc1cc(C2CC2)cc2c1-c1cc(c(F)c(C(F)(F)F)c1)[C@H](CC(=O)O)NC(=O)[C@@H](n1cc(CCN3CC(F)C3)c(C(F)(F)F)cc1=O)c1cccc(c1)O2 ; Cc1cc(Cl)cc2c1-c1cc(c(F)c(C(F)(F)F)c1)[C@H](CC(=O)O)NC(=O)[C@@H](n1cc(CCN3CC(F)C3)c(C(F)(F)F)cc1=O)c1cccc(c1)O2 ; Cc1ccc(C)c2c1Oc1cccc(c1)[C@H](n1cc(CCN3CC(F)C3)c(C(F)(F)F)cc1=O)C(=O)N[C@@H](CC(=O)O)c1cc-2cc(C(F)(F)F)c1F]

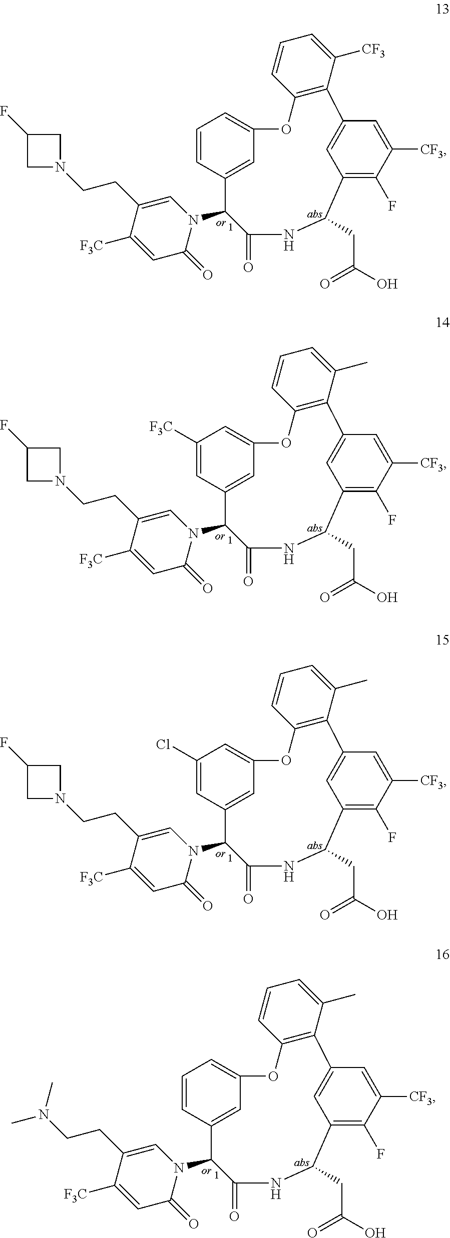 [SMILES: O=C(O)C[C@@H]1NC(=O)[C@@H](n2cc(CCN3CC(F)C3)c(C(F)(F)F)cc2=O)c2cccc(c2)Oc2cccc(C(F)(F)F)c2-c2cc1c(F)c(C(F)(F)F)c2 ; Cc1cccc2c1-c1cc(c(F)c(C(F)(F)F)c1)[C@H](CC(=O)O)NC(=O)[C@@H](n1cc(CCN3CC(F)C3)c(C(F)(F)F)cc1=O)c1cc(cc(C(F)(F)F)c1)O2 ; Cc1cccc2c1-c1cc(c(F)c(C(F)(F)F)c1)[C@H](CC(=O)O)NC(=O)[C@@H](n1cc(CCN3CC(F)C3)c(C(F)(F)F)cc1=O)c1cc(Cl)cc(c1)O2 ; Cc1cccc2c1-c1cc(c(F)c(C(F)(F)F)c1)[C@H](CC(=O)O)NC(=O)[C@@H](n1cc(CCN(C)C)c(C(F)(F)F)cc1=O)c1cccc(c1)O2]

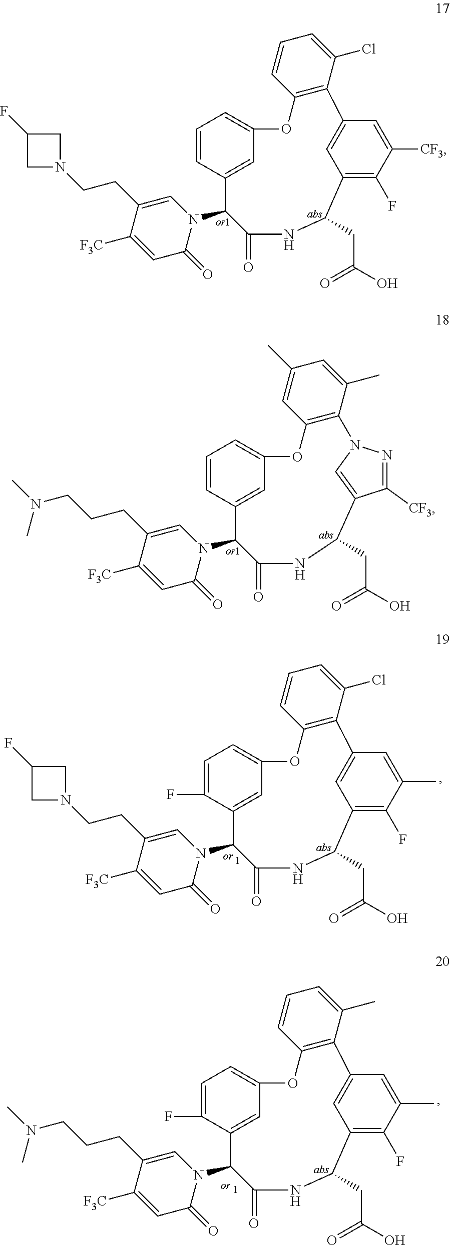

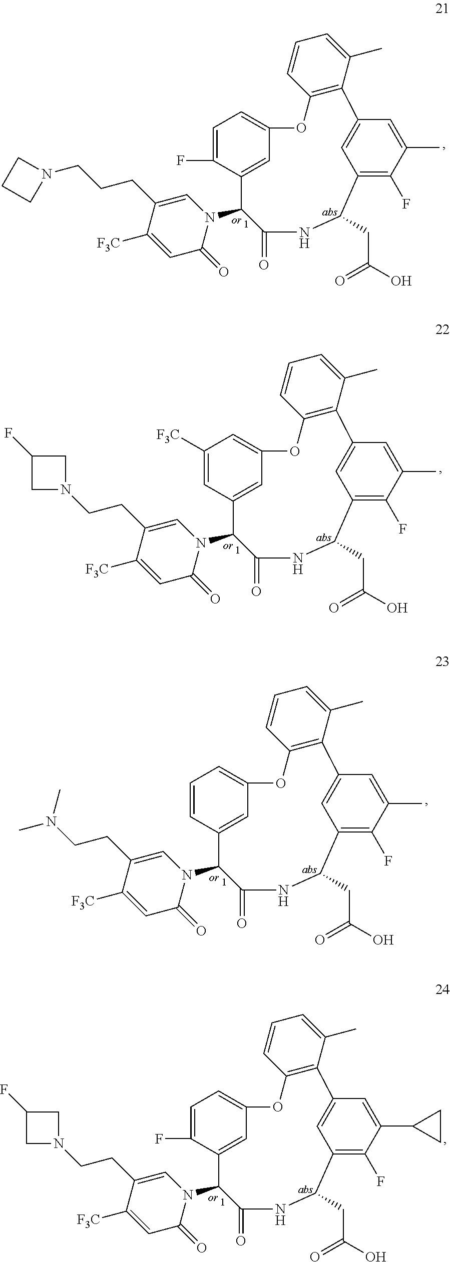

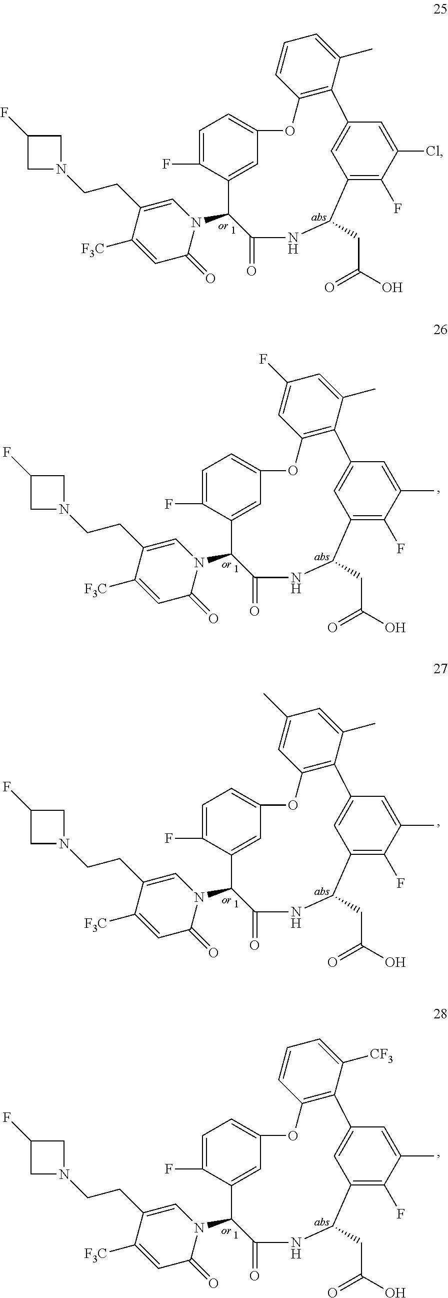 [SMILES: Cc1cccc2c1-c1cc(Cl)c(F)c(c1)[C@H](CC(=O)O)NC(=O)[C@@H](n1cc(CCN3CC(F)C3)c(C(F)(F)F)cc1=O)c1cc(ccc1F)O2 ; Cc1cc2cc(c1F)[C@H](CC(=O)O)NC(=O)[C@@H](n1cc(CCN3CC(F)C3)c(C(F)(F)F)cc1=O)c1cc(ccc1F)Oc1cc(F)cc(C)c1-2 ; Cc1cc(C)c2c(c1)Oc1ccc(F)c(c1)[C@H](n1cc(CCN3CC(F)C3)c(C(F)(F)F)cc1=O)C(=O)N[C@@H](CC(=O)O)c1cc-2cc(C)c1F ; Cc1cc2cc(c1F)[C@H](CC(=O)O)NC(=O)[C@@H](n1cc(CCN3CC(F)C3)c(C(F)(F)F)cc1=O)c1cc(ccc1F)Oc1cccc(C(F)(F)F)c1-2]

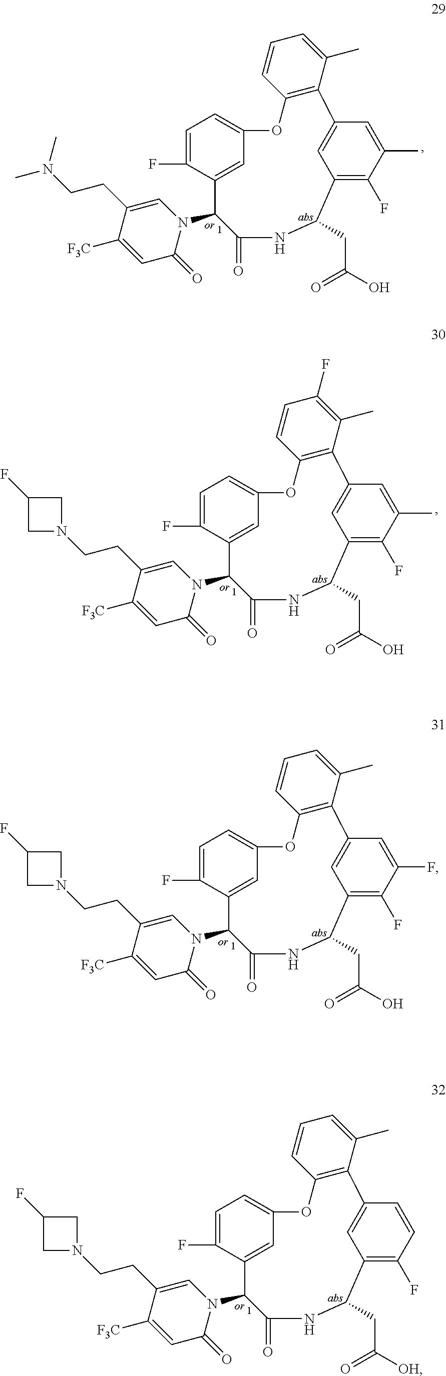

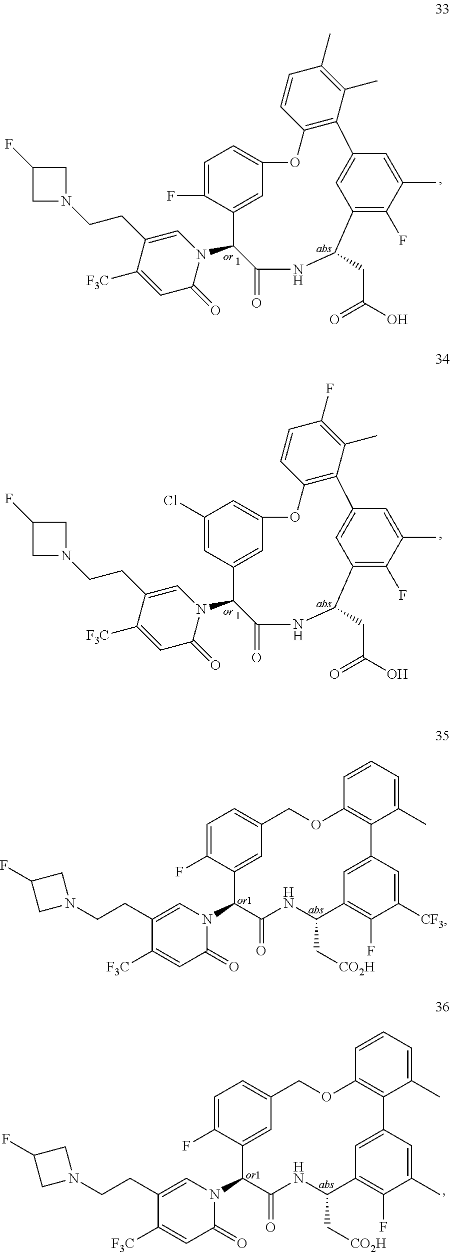 [SMILES: Cc1ccc2c(c1C)-c1cc(C)c(F)c(c1)[C@H](CC(=O)O)NC(=O)[C@@H](n1cc(CCN3CC(F)C3)c(C(F)(F)F)cc1=O)c1cc(ccc1F)O2 ; Cc1cc2cc(c1F)[C@H](CC(=O)O)NC(=O)[C@@H](n1cc(CCN3CC(F)C3)c(C(F)(F)F)cc1=O)c1cc(Cl)cc(c1)Oc1ccc(F)c(C)c1-2 ; Cc1cccc2c1-c1cc(c(F)c(C(F)(F)F)c1)[C@H](CC(=O)O)NC(=O)[C@@H](n1cc(CCN3CC(F)C3)c(C(F)(F)F)cc1=O)c1cc(ccc1F)CO2 ; Cc1cc2cc(c1F)[C@H](CC(=O)O)NC(=O)[C@@H](n1cc(CCN3CC(F)C3)c(C(F)(F)F)cc1=O)c1cc(ccc1F)COc1cccc(C)c1-2]

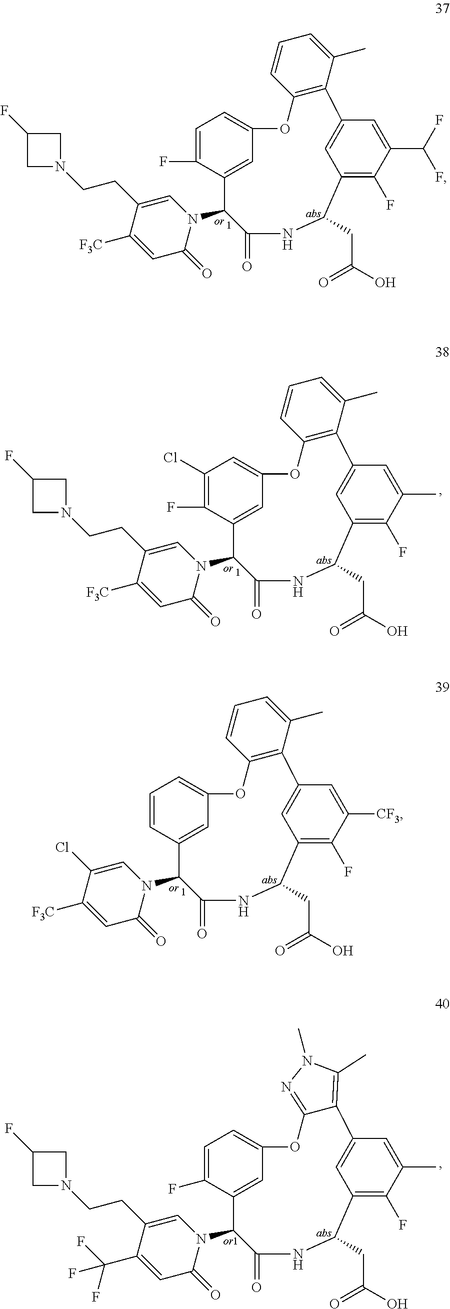 [SMILES: Cc1cccc2c1-c1cc(C(F)F)c(F)c(c1)[C@H](CC(=O)O)NC(=O)[C@@H](n1cc(CCN3CC(F)C3)c(C(F)(F)F)cc1=O)c1cc(ccc1F)O2 ; Cc1cc2cc(c1F)[C@H](CC(=O)O)NC(=O)[C@@H](n1cc(CCN3CC(F)C3)c(C(F)(F)F)cc1=O)c1cc(cc(Cl)c1F)Oc1cccc(C)c1-2 ; Cc1cccc2c1-c1cc(c(F)c(C(F)(F)F)c1)[C@H](CC(=O)O)NC(=O)[C@@H](n1cc(Cl)c(C(F)(F)F)cc1=O)c1cccc(c1)O2 ; Cc1cc2cc(c1F)[C@H](CC(=O)O)NC(=O)[C@@H](n1cc(CCN3CC(F)C3)c(C(F)(F)F)cc1=O)c1cc(ccc1F)Oc1nn(C)c(C)c1-2]

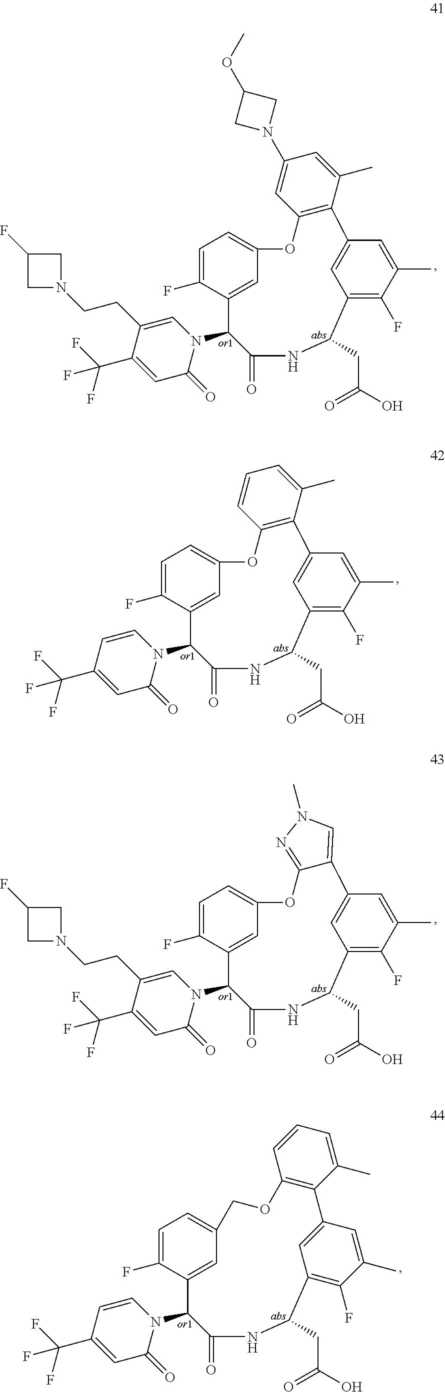 [SMILES: COC1CN(c2cc(C)c3c(c2)Oc2ccc(F)c(c2)[C@H](n2cc(CCN4CC(F)C4)c(C(F)(F)F)cc2=O)C(=O)N[C@@H](CC(=O)O)c2cc-3cc(C)c2F)C1 ; Cc1cc2cc(c1F)[C@H](CC(=O)O)NC(=O)[C@@H](n1ccc(C(F)(F)F)cc1=O)c1cc(ccc1F)Oc1cccc(C)c1-2 ; Cc1cc2cc(c1F)[C@H](CC(=O)O)NC(=O)[C@@H](n1cc(CCN3CC(F)C3)c(C(F)(F)F)cc1=O)c1cc(ccc1F)Oc1nn(C)cc1-2 ; Cc1cc2cc(c1F)[C@H](CC(=O)O)NC(=O)[C@@H](n1ccc(C(F)(F)F)cc1=O)c1cc(ccc1F)COc1cccc(C)c1-2]

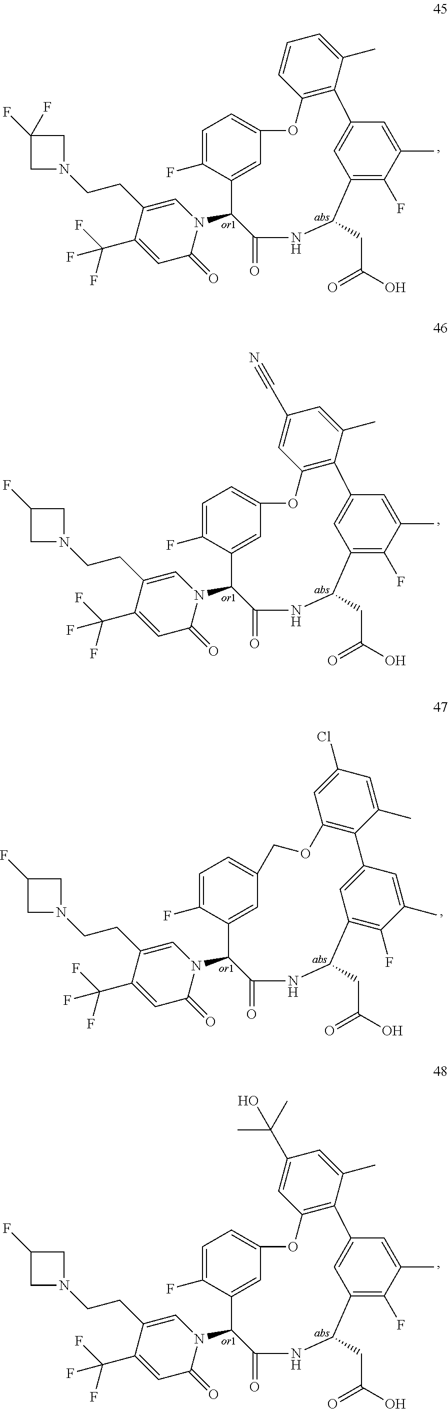 [SMILES: Cc1cc2cc(c1F)[C@H](CC(=O)O)NC(=O)[C@@H](n1cc(CCN3CC(F)(F)C3)c(C(F)(F)F)cc1=O)c1cc(ccc1F)Oc1cccc(C)c1-2 ; Cc1cc2cc(c1F)[C@H](CC(=O)O)NC(=O)[C@@H](n1cc(CCN3CC(F)C3)c(C(F)(F)F)cc1=O)c1cc(ccc1F)Oc1cc(C#N)cc(C)c1-2 ; Cc1cc2cc(c1F)[C@H](CC(=O)O)NC(=O)[C@@H](n1cc(CCN3CC(F)C3)c(C(F)(F)F)cc1=O)c1cc(ccc1F)COc1cc(Cl)cc(C)c1-2 ; Cc1cc2cc(c1F)[C@H](CC(=O)O)NC(=O)[C@@H](n1cc(CCN3CC(F)C3)c(C(F)(F)F)cc1=O)c1cc(ccc1F)Oc1cc(C(C)(C)O)cc(C)c1-2]

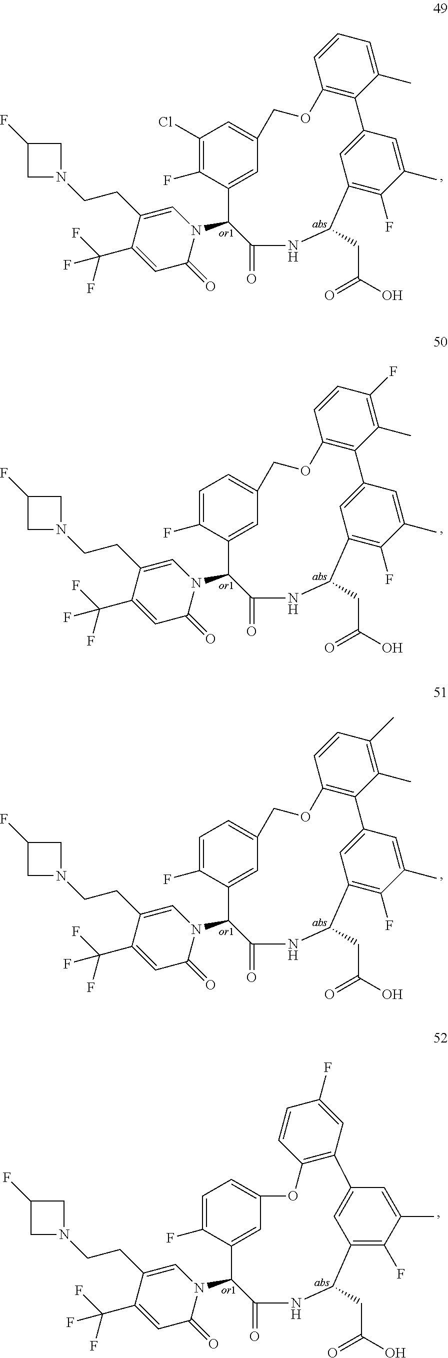 [SMILES: Cc1cc2cc(c1F)[C@H](CC(=O)O)NC(=O)[C@@H](n1cc(CCN3CC(F)C3)c(C(F)(F)F)cc1=O)c1cc(cc(Cl)c1F)COc1cccc(C)c1-2 ; Cc1cc2cc(c1F)[C@H](CC(=O)O)NC(=O)[C@@H](n1cc(CCN3CC(F)C3)c(C(F)(F)F)cc1=O)c1cc(ccc1F)COc1ccc(F)c(C)c1-2 ; Cc1ccc2c(c1C)-c1cc(C)c(F)c(c1)[C@H](CC(=O)O)NC(=O)[C@@H](n1cc(CCN3CC(F)C3)c(C(F)(F)F)cc1=O)c1cc(ccc1F)CO2 ; Cc1cc2cc(c1F)[C@H](CC(=O)O)NC(=O)[C@@H](n1cc(CCN3CC(F)C3)c(C(F)(F)F)cc1=O)c1cc(ccc1F)Oc1ccc(F)cc1-2]

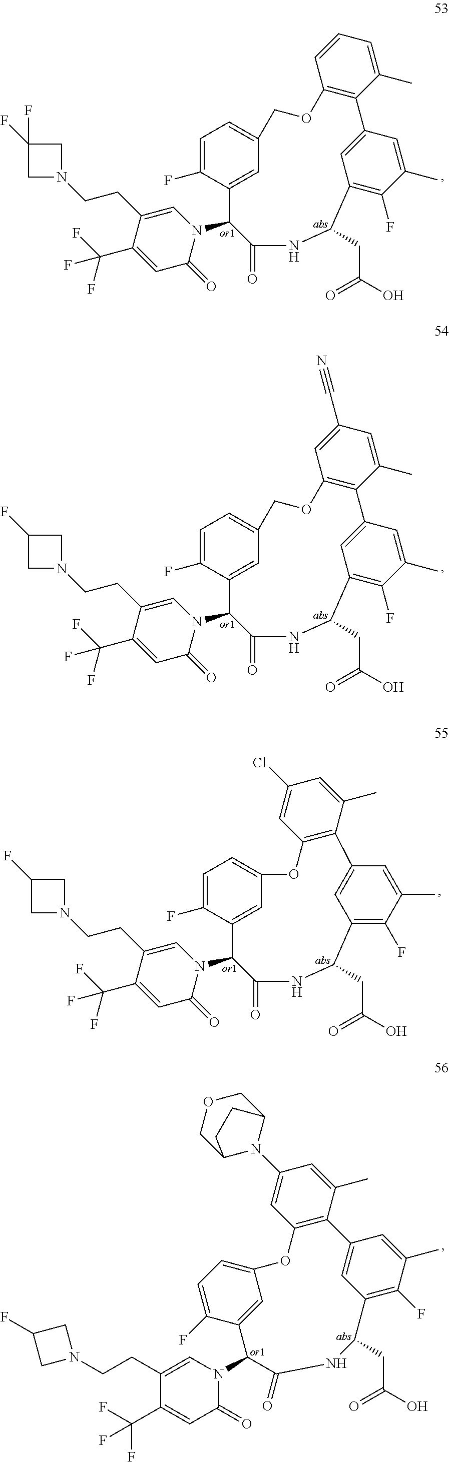 [SMILES: Cc1cc2cc(c1F)[C@H](CC(=O)O)NC(=O)[C@@H](n1cc(CCN3CC(F)(F)C3)c(C(F)(F)F)cc1=O)c1cc(ccc1F)COc1cccc(C)c1-2 ; Cc1cc2cc(c1F)[C@H](CC(=O)O)NC(=O)[C@@H](n1cc(CCN3CC(F)C3)c(C(F)(F)F)cc1=O)c1cc(ccc1F)COc1cc(C#N)cc(C)c1-2 ; Cc1cc2cc(c1F)[C@H](CC(=O)O)NC(=O)[C@@H](n1cc(CCN3CC(F)C3)c(C(F)(F)F)cc1=O)c1cc(ccc1F)Oc1cc(Cl)cc(C)c1-2 ; Cc1cc2cc(c1F)[C@H](CC(=O)O)NC(=O)[C@@H](n1cc(CCN3CC(F)C3)c(C(F)(F)F)cc1=O)c1cc(ccc1F)Oc1cc(N3C4CCC3COC4)cc(C)c1-2]

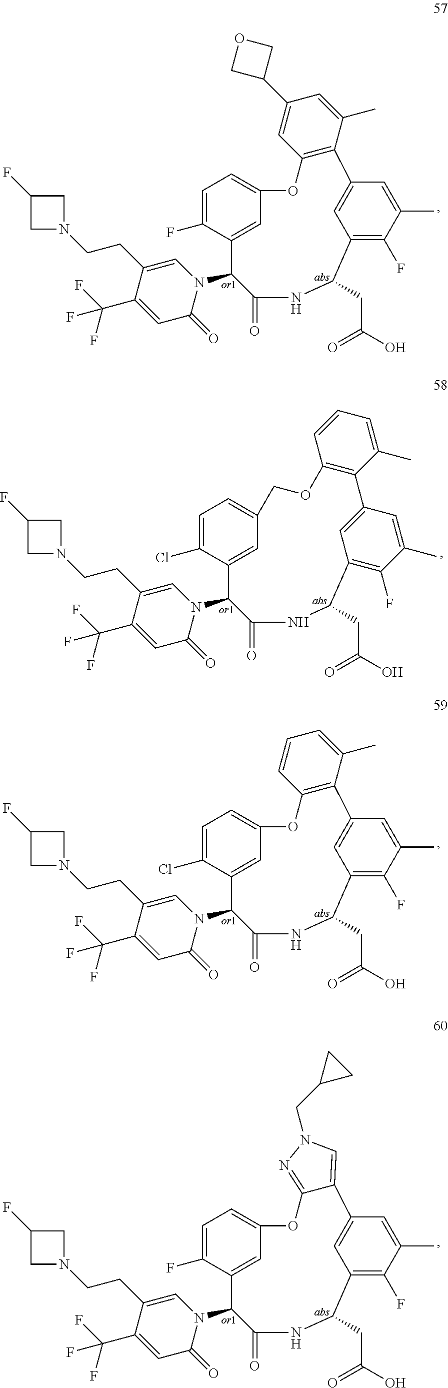 [SMILES: Cc1cc2cc(c1F)[C@H](CC(=O)O)NC(=O)[C@@H](n1cc(CCN3CC(F)C3)c(C(F)(F)F)cc1=O)c1cc(ccc1F)Oc1cc(C3COC3)cc(C)c1-2 ; Cc1cc2cc(c1F)[C@H](CC(=O)O)NC(=O)[C@@H](n1cc(CCN3CC(F)C3)c(C(F)(F)F)cc1=O)c1cc(ccc1Cl)COc1cccc(C)c1-2 ; Cc1cc2cc(c1F)[C@H](CC(=O)O)NC(=O)[C@@H](n1cc(CCN3CC(F)C3)c(C(F)(F)F)cc1=O)c1cc(ccc1Cl)Oc1cccc(C)c1-2 ; Cc1cc2cc(c1F)[C@H](CC(=O)O)NC(=O)[C@@H](n1cc(CCN3CC(F)C3)c(C(F)(F)F)cc1=O)c1cc(ccc1F)Oc1nn(CC3CC3)cc1-2]

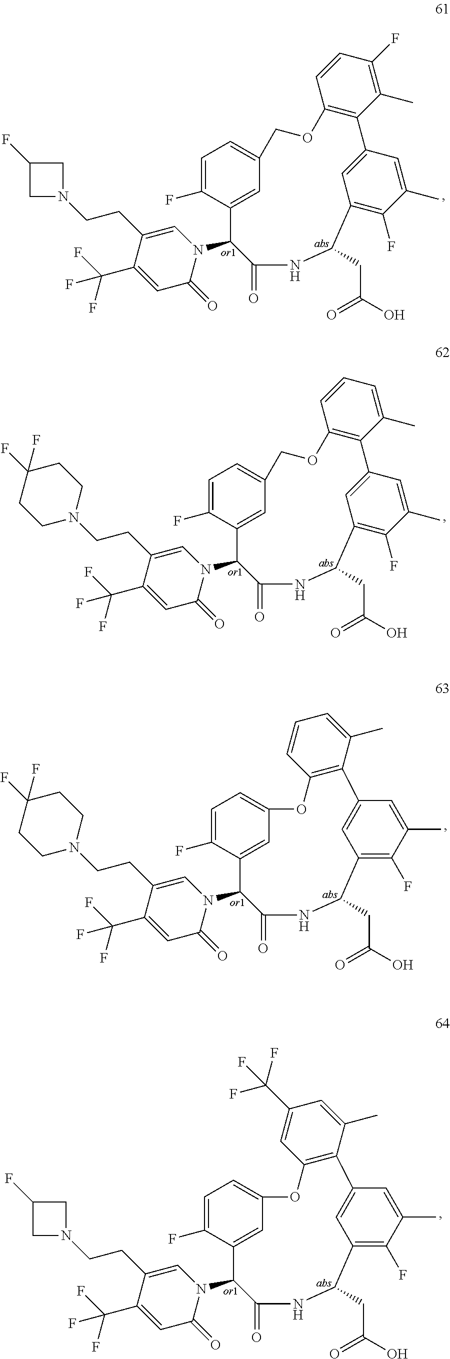 [SMILES: Cc1cc2cc(c1F)[C@H](CC(=O)O)NC(=O)[C@@H](n1cc(CCN3CC(F)C3)c(C(F)(F)F)cc1=O)c1cc(ccc1F)COc1ccc(F)c(C)c1-2 ; Cc1cc2cc(c1F)[C@H](CC(=O)O)NC(=O)[C@@H](n1cc(CCN3CCC(F)(F)CC3)c(C(F)(F)F)cc1=O)c1cc(ccc1F)COc1cccc(C)c1-2 ; Cc1cc2cc(c1F)[C@H](CC(=O)O)NC(=O)[C@@H](n1cc(CCN3CCC(F)(F)CC3)c(C(F)(F)F)cc1=O)c1cc(ccc1F)Oc1cccc(C)c1-2 ; Cc1cc2cc(c1F)[C@H](CC(=O)O)NC(=O)[C@@H](n1cc(CCN3CC(F)C3)c(C(F)(F)F)cc1=O)c1cc(ccc1F)Oc1cc(C(F)(F)F)cc(C)c1-2]

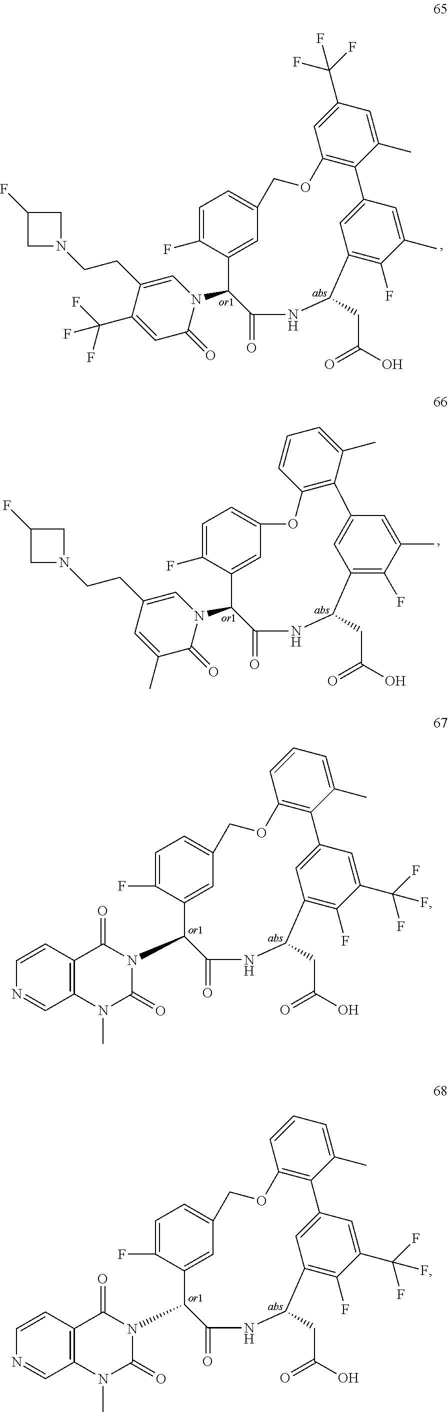 [SMILES: Cc1cc2cc(c1F)[C@H](CC(=O)O)NC(=O)[C@@H](n1cc(CCN3CC(F)C3)c(C(F)(F)F)cc1=O)c1cc(ccc1F)COc1cc(C(F)(F)F)cc(C)c1-2 ; Cc1cc2cc(c1F)[C@H](CC(=O)O)NC(=O)[C@@H](n1cc(CCN3CC(F)C3)cc(C)c1=O)c1cc(ccc1F)Oc1cccc(C)c1-2 ; Cc1cccc2c1-c1cc(c(F)c(C(F)(F)F)c1)[C@H](CC(=O)O)NC(=O)[C@@H](n1c(=O)c3ccncc3n(C)c1=O)c1cc(ccc1F)CO2 ; Cc1cccc2c1-c1cc(c(F)c(C(F)(F)F)c1)[C@H](CC(=O)O)NC(=O)[C@H](n1c(=O)c3ccncc3n(C)c1=O)c1cc(ccc1F)CO2]

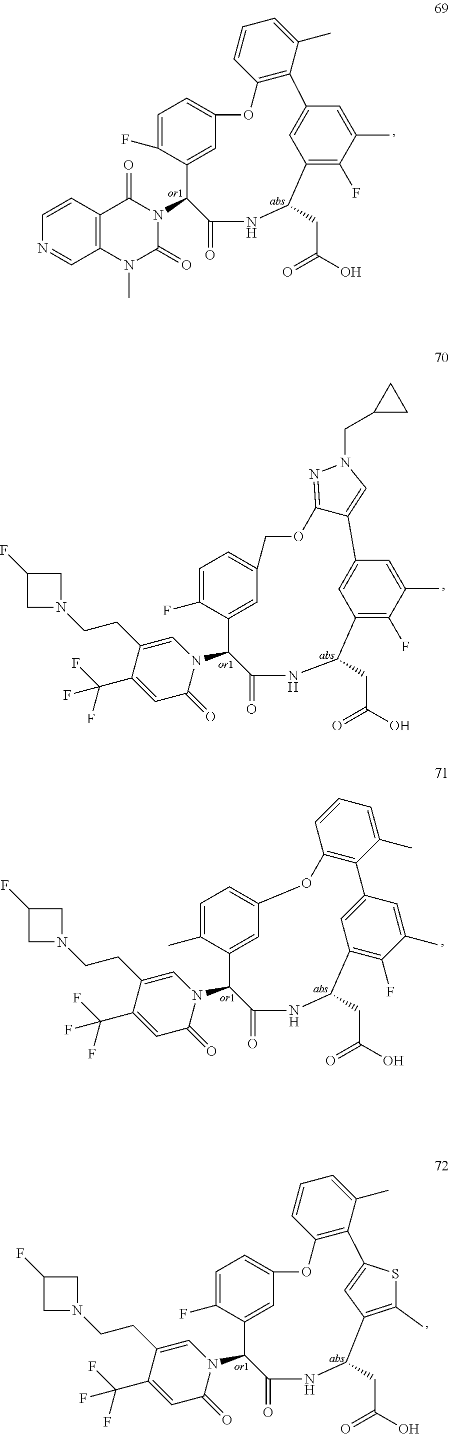 [SMILES: Cc1cc2cc(c1F)[C@H](CC(=O)O)NC(=O)[C@@H](n1c(=O)c3ccncc3n(C)c1=O)c1cc(ccc1F)Oc1cccc(C)c1-2 ; Cc1cc2cc(c1F)[C@H](CC(=O)O)NC(=O)[C@@H](n1cc(CCN3CC(F)C3)c(C(F)(F)F)cc1=O)c1cc(ccc1F)COc1nn(CC3CC3)cc1-2 ; Cc1ccc2cc1[C@H](n1cc(CCN3CC(F)C3)c(C(F)(F)F)cc1=O)C(=O)N[C@@H](CC(=O)O)c1cc(cc(C)c1F)-c1c(C)cccc1O2 ; Cc1cccc2c1-c1cc(c(C)s1)[C@H](CC(=O)O)NC(=O)[C@@H](n1cc(CCN3CC(F)C3)c(C(F)(F)F)cc1=O)c1cc(ccc1F)O2]

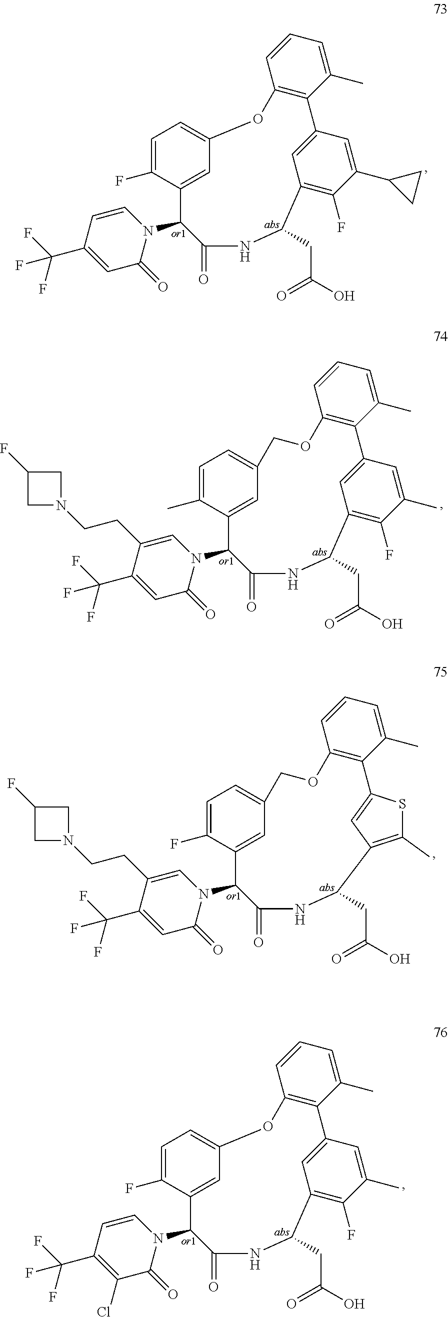 [SMILES: Cc1cccc2c1-c1cc(C3CC3)c(F)c(c1)[C@H](CC(=O)O)NC(=O)[C@@H](n1ccc(C(F)(F)F)cc1=O)c1cc(ccc1F)O2 ; Cc1ccc2cc1[C@H](n1cc(CCN3CC(F)C3)c(C(F)(F)F)cc1=O)C(=O)N[C@@H](CC(=O)O)c1cc(cc(C)c1F)-c1c(C)cccc1OC2 ; Cc1cccc2c1-c1cc(c(C)s1)[C@H](CC(=O)O)NC(=O)[C@@H](n1cc(CCN3CC(F)C3)c(C(F)(F)F)cc1=O)c1cc(ccc1F)CO2 ; Cc1cc2cc(c1F)[C@H](CC(=O)O)NC(=O)[C@@H](n1ccc(C(F)(F)F)c(Cl)c1=O)c1cc(ccc1F)Oc1cccc(C)c1-2]

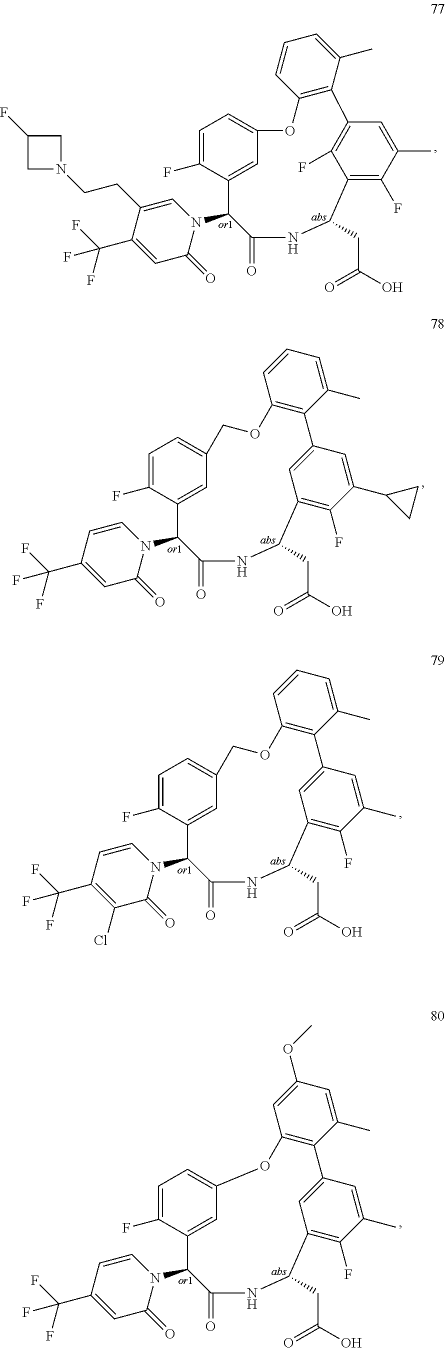 [SMILES: Cc1cc2c(F)c(c1F)[C@H](CC(=O)O)NC(=O)[C@@H](n1cc(CCN3CC(F)C3)c(C(F)(F)F)cc1=O)c1cc(ccc1F)Oc1cccc(C)c1-2 ; Cc1cccc2c1-c1cc(C3CC3)c(F)c(c1)[C@H](CC(=O)O)NC(=O)[C@@H](n1ccc(C(F)(F)F)cc1=O)c1cc(ccc1F)CO2 ; Cc1cc2cc(c1F)[C@H](CC(=O)O)NC(=O)[C@@H](n1ccc(C(F)(F)F)c(Cl)c1=O)c1cc(ccc1F)COc1cccc(C)c1-2 ; COc1cc(C)c2c(c1)Oc1ccc(F)c(c1)[C@H](n1ccc(C(F)(F)F)cc1=O)C(=O)N[C@@H](CC(=O)O)c1cc-2cc(C)c1F]

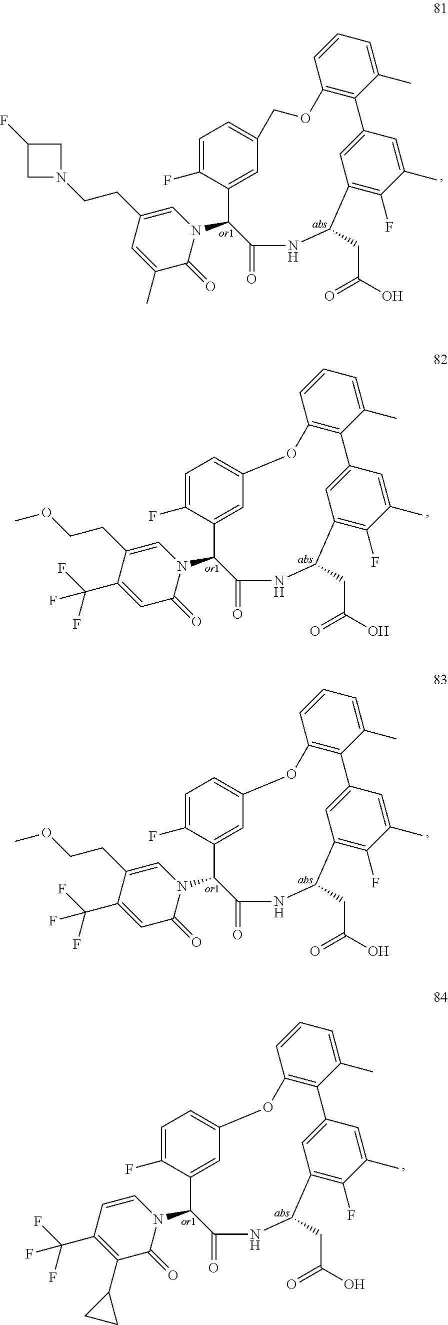

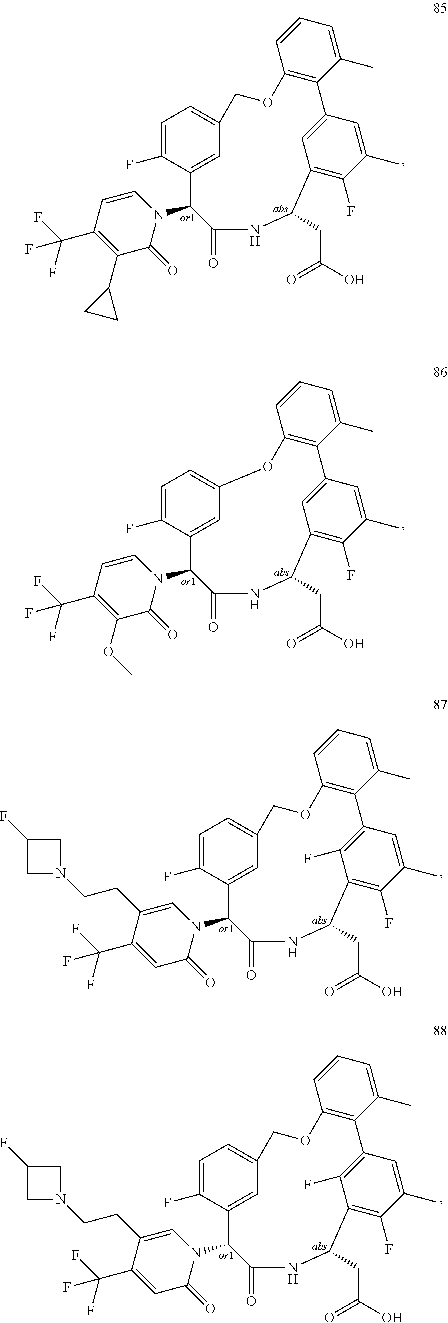 [SMILES: Cc1cc2cc(c1F)[C@H](CC(=O)O)NC(=O)[C@@H](n1ccc(C(F)(F)F)c(C3CC3)c1=O)c1cc(ccc1F)COc1cccc(C)c1-2 ; COc1c(C(F)(F)F)ccn([C@@H]2C(=O)N[C@@H](CC(=O)O)c3cc(cc(C)c3F)-c3c(C)cccc3Oc3ccc(F)c2c3)c1=O ; Cc1cc2c(F)c(c1F)[C@H](CC(=O)O)NC(=O)[C@@H](n1cc(CCN3CC(F)C3)c(C(F)(F)F)cc1=O)c1cc(ccc1F)COc1cccc(C)c1-2 ; Cc1cc2c(F)c(c1F)[C@H](CC(=O)O)NC(=O)[C@H](n1cc(CCN3CC(F)C3)c(C(F)(F)F)cc1=O)c1cc(ccc1F)COc1cccc(C)c1-2]

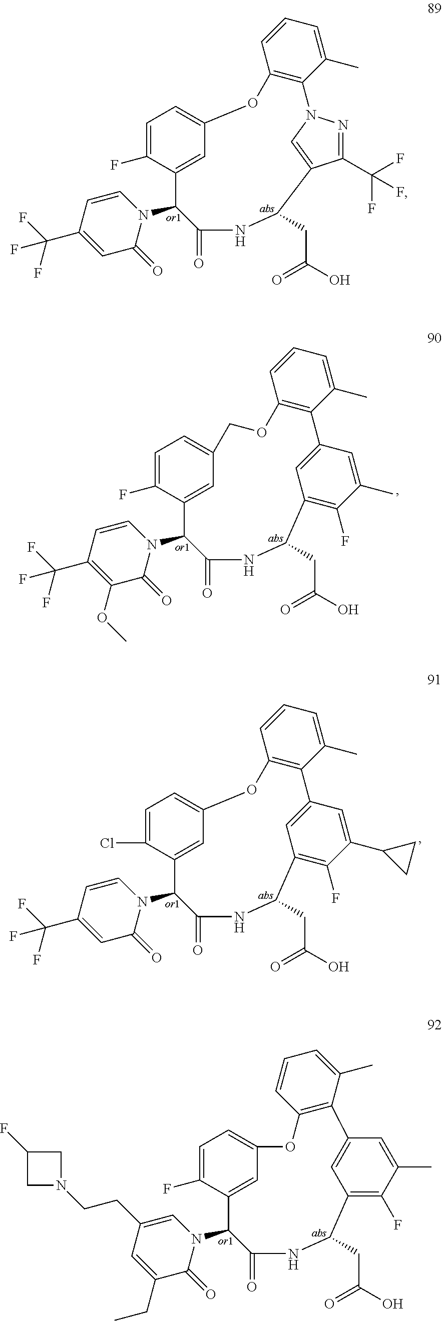 [SMILES: Cc1cccc2c1-n1cc(c(C(F)(F)F)n1)[C@H](CC(=O)O)NC(=O)[C@@H](n1ccc(C(F)(F)F)cc1=O)c1cc(ccc1F)O2 ; COc1c(C(F)(F)F)ccn([C@@H]2C(=O)N[C@@H](CC(=O)O)c3cc(cc(C)c3F)-c3c(C)cccc3OCc3ccc(F)c2c3)c1=O ; Cc1cccc2c1-c1cc(C3CC3)c(F)c(c1)[C@H](CC(=O)O)NC(=O)[C@@H](n1ccc(C(F)(F)F)cc1=O)c1cc(ccc1Cl)O2 ; CCc1cc(CCN2CC(F)C2)cn([C@@H]2C(=O)N[C@@H](CC(=O)O)c3cc(cc(C)c3F)-c3c(C)cccc3Oc3ccc(F)c2c3)c1=O]

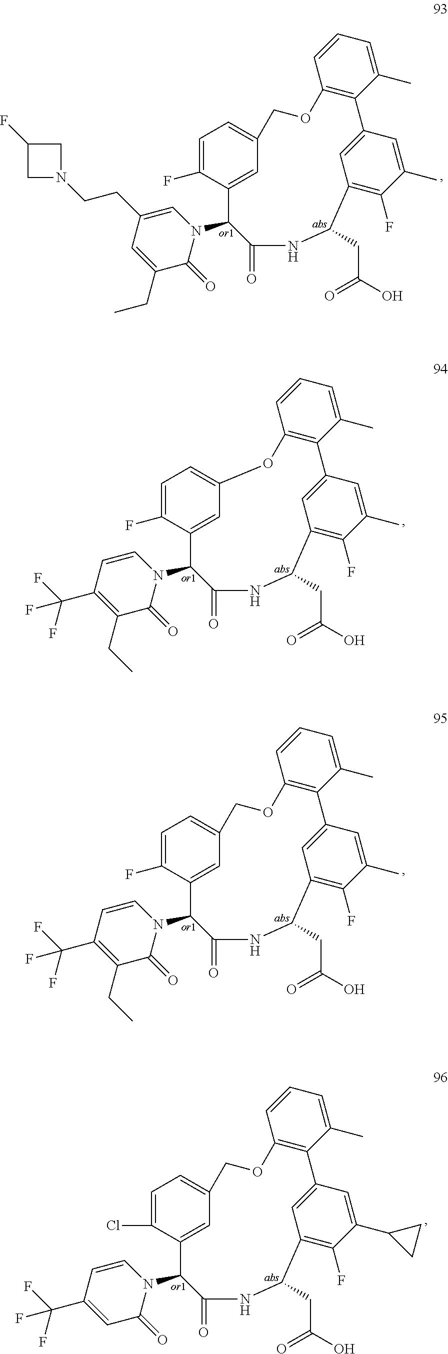 [SMILES: CCc1cc(CCN2CC(F)C2)cn([C@@H]2C(=O)N[C@@H](CC(=O)O)c3cc(cc(C)c3F)-c3c(C)cccc3OCc3ccc(F)c2c3)c1=O ; CCc1c(C(F)(F)F)ccn([C@@H]2C(=O)N[C@@H](CC(=O)O)c3cc(cc(C)c3F)-c3c(C)cccc3Oc3ccc(F)c2c3)c1=O ; CCc1c(C(F)(F)F)ccn([C@@H]2C(=O)N[C@@H](CC(=O)O)c3cc(cc(C)c3F)-c3c(C)cccc3OCc3ccc(F)c2c3)c1=O ; Cc1cccc2c1-c1cc(C3CC3)c(F)c(c1)[C@H](CC(=O)O)NC(=O)[C@@H](n1ccc(C(F)(F)F)cc1=O)c1cc(ccc1Cl)CO2]

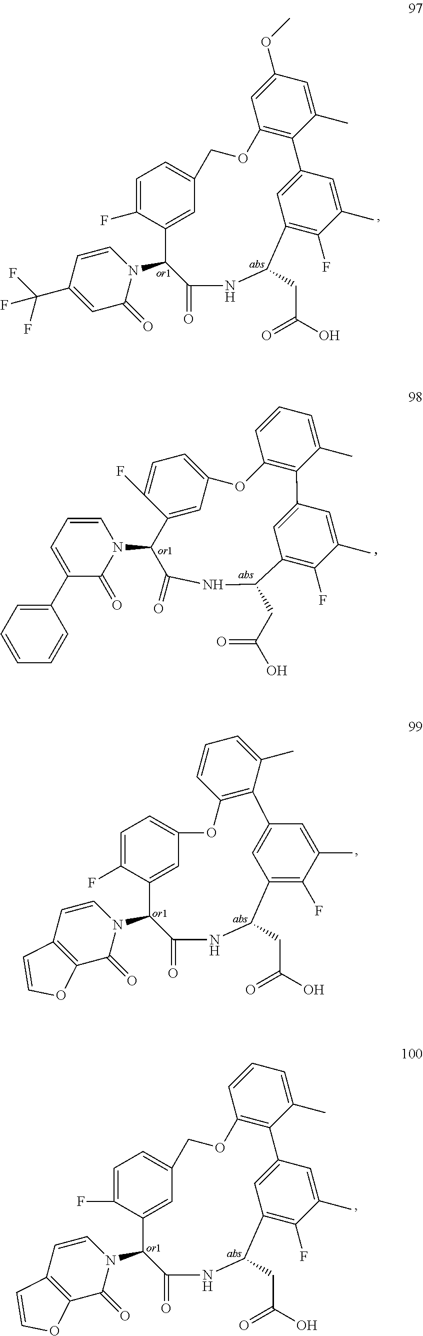 [SMILES: COc1cc(C)c2c(c1)OCc1ccc(F)c(c1)[C@H](n1ccc(C(F)(F)F)cc1=O)C(=O)N[C@@H](CC(=O)O)c1cc-2cc(C)c1F ; Cc1cc2cc(c1F)[C@H](CC(=O)O)NC(=O)[C@@H](n1cccc(-c3ccccc3)c1=O)c1cc(ccc1F)Oc1cccc(C)c1-2 ; Cc1cc2cc(c1F)[C@H](CC(=O)O)NC(=O)[C@@H](n1ccc3ccoc3c1=O)c1cc(ccc1F)Oc1cccc(C)c1-2 ; Cc1cc2cc(c1F)[C@H](CC(=O)O)NC(=O)[C@@H](n1ccc3ccoc3c1=O)c1cc(ccc1F)COc1cccc(C)c1-2]

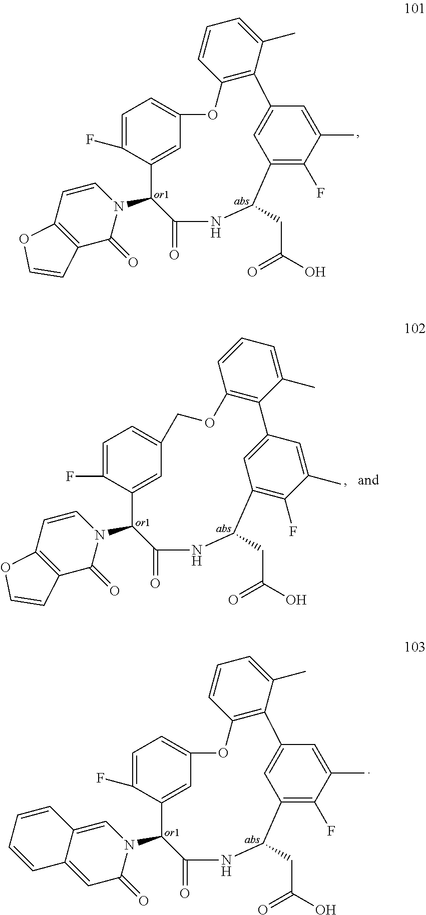 [SMILES: Cc1cc2cc(c1F)[C@H](CC(=O)O)NC(=O)[C@@H](n1ccc3occc3c1=O)c1cc(ccc1F)Oc1cccc(C)c1-2 ; Cc1cc2cc(c1F)[C@H](CC(=O)O)NC(=O)[C@@H](n1ccc3occc3c1=O)c1cc(ccc1F)COc1cccc(C)c1-2 ; Cc1cc2cc(c1F)[C@H](CC(=O)O)NC(=O)[C@@H](n1cc3ccccc3cc1=O)c1cc(ccc1F)Oc1cccc(C)c1-2]

69. A pharmaceutical composition comprising a compound of claim 1 or a pharmaceutically acceptable salt, solvate, or stereoisomer thereof, and a pharmaceutically acceptable excipient.

70. A method of treating a disease, the method comprising administering a compound of claim 1, or a pharmaceutically acceptable salt, solvate, or stereoisomer thereof.

71. The method of claim 70, wherein the disease is inflammatory bowel disease, ileoanal anastomosis, eosinophilic esophagitis, pancreatitis, insulin-dependent diabetes mellitus, mastitis, cholecystitis, cholangitis, pericholangitis, chronic bronchitis, chronic sinusitis, asthma, graft versus host disease, or a chronic inflammatory disease of the lung.

72-82. (canceled)
